(19) United States
(12) Patent Application Publication
(10) Pub. No.: US 2023/0330254 A1
(43) Pub. Date: Oct. 19, 2023

(54) ANTI-GLP1R ANTIBODY-TETHERED DRUG CONJUGATES COMPRISING GLP1 PEPTIDOMIMETICS AND USES THEREOF

(71) Applicant: Regeneron Pharmaceuticals, Inc., Tarrytown, NY (US)

(72) Inventors: Haruka OKAMOTO, Ardsley, NY (US); Jean YANOLATOS, Bronx, NY (US); AMY HAN, Hockessin, DE (US); Xiang ZHENG, Allendale, NJ (US); William OLSON, Yorktown Heights, NY (US); Andrew J. MURPHY, Croton-on-Hudson, NY (US); Frank DELFINO, Poughquag, NY (US)

(73) Assignee: Regeneron Pharmaceuticals, Inc., Tarrytown, NY (US)

(21) Appl. No.: 18/182,323

(22) Filed: Mar. 11, 2023

Related U.S. Application Data
(60) Provisional application No. 63/319,175, filed on Mar. 11, 2022.

(51) Int. Cl. A61K 47/68; A61K 47/54; A61P 3/10

(52) U.S. Cl. CPC A61K 47/6849; A61K 47/6811; A61K 47/545; A61P 3/10; A61K 2039/505

(57) ABSTRACT

The present invention provides antibody-tethered drug conjugates (ATDCs) and compositions thereof that are useful, for example, for targeting glucagon-like peptide 1 receptor (GLP1R) and treating various conditions, e.g., diabetes. Methods for making such ATDCs are also provided along with method of use thereof.

### CROSS REFERENCE TO RELATED APPLICATIONS

This application claims priority to U.S. Provisional Application No. 63/319,175, filed Mar. 11, 2022, which is hereby incorporated by reference in its entirety.

### SEQUENCE LISTING

The instant application contains a Sequence Listing which has been submitted electronically in XML file format and is hereby incorporated by reference in its entirety. Said XML copy, created on Jun. 23, 2023, is named 250298_000475_SL.xml and is 852,456 bytes in size.

### FIELD OF THE DISCLOSURE

The present disclosure relates to antibody-tethered drug conjugates, pharmaceutical compositions, and methods of treating GLP1R-associated conditions therewith.

### BACKGROUND OF THE DISCLOSURE

Diabetes is a chronic disease of abnormal glucose metabolism. 425 million people are estimated to be living with diabetes worldwide. Global diabetes drugs include insulin, DPP-4 inhibitors, glucagon-like peptide 1 receptor (GLP1R) agonists, but most patients do not achieve combined treatment goal to manage hyperglycaemia and cardiovascular risk factors.

Glucagon-Like Peptide 1 Receptor (GLP1R) is the receptor for glucagon-like peptide 1 (GLP1) and is expressed in the pancreatic beta cells. GLP1R is also expressed in the brain where it functions in the control of appetite, memory and learning. GLP1R is a member of the secretin family (Class B) of G protein-coupled receptors (GPCRs). Upon binding of its ligand, GLP1, GLP1R initiates a downstream signaling cascade through Gαs G-proteins that raises intracellular cyclic AMP (cAMP) levels, which leads to the transcriptional regulation of genes (Donnelly 2011). Activation of GLP1R results in increased insulin synthesis and release of insulin.

GLP1R and GLP1 are highly validated targets for obesity and type 2 diabetes. Marketed GLP1R agonists increase insulin secretion, thereby lowering blood glucose levels, but they require weekly or more frequent administration.

Accordingly, there is a need in the art for GLP1R agonists with longer duration and better safety. In certain embodiments, the present disclosure meets the needs and provides other advantages.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE DISCLOSURE

Various non-limiting aspects and embodiments of the disclosure are described below.

The present invention provides an antibody-tethered drug conjugate (ATDC) having a structure of Formula (A):

BA-(L-P)ₘ (A),

wherein:
BA is an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
L is a non-cleavable linker;
optionally, wherein the heavy chain immunoglobulin of the BA does not comprise a C-terminal lysine or lysine and glycine;
P is a payload having the structure selected from the group consisting of (SEQ ID NOS 448-450, respectively, in order of appearance):

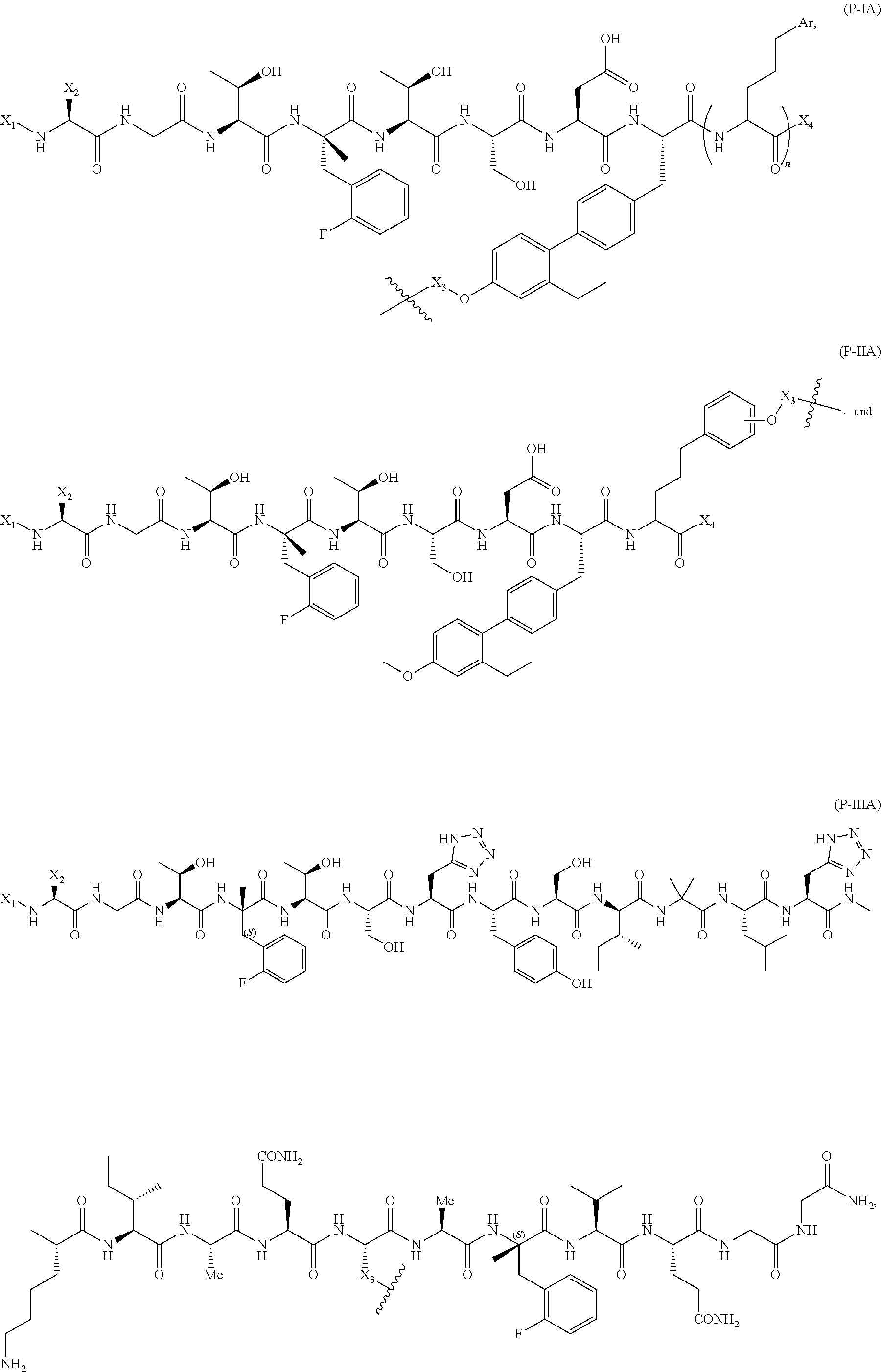

wherein

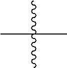

is the point of attachment of the payload to L;
X₁ is selected from H;

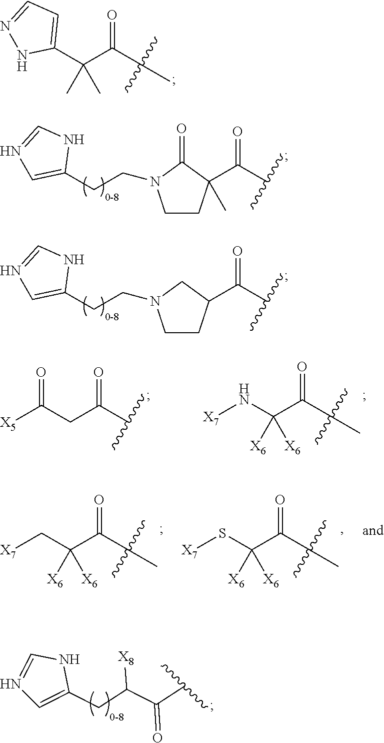

X₂ is selected from

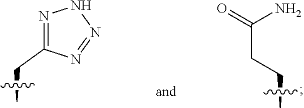

X₃ is selected from a bond, —(CH₂)₂₋₆—NH—, —(CH₂)₂₋₆-Tr-, and —(CH₂)₂₋₆-Tr-(CH₂)₁₋₆—NH, where Tr is a triazole moiety;
n is 0 or 1;
X₄ is selected from —NH₂, —OH and —N(H)(phenyl);
X₅ is selected from —OH, —NH₂, —NH—OH, and

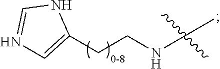

X₆ is independently at each occurrence selected from H, —OH, —CH₃, and —CH₂OH;
X₇ is selected from H,

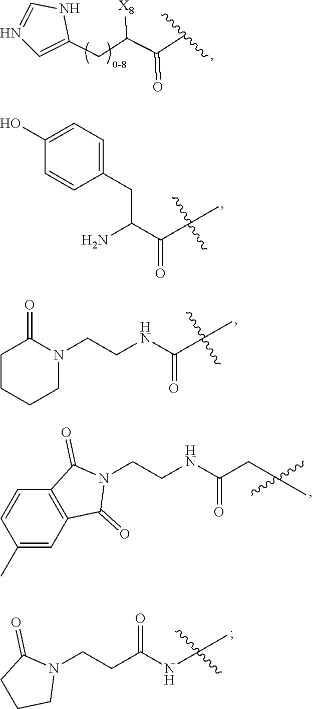

X₈ is selected from H, —OH, —NH₂, and

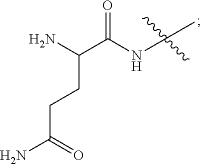 [SMILES: *NC(=O)C(N)CCC(N)=O]

Ar is selected from

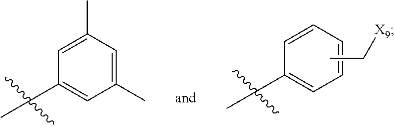

X₉ is selected from —NH₂,

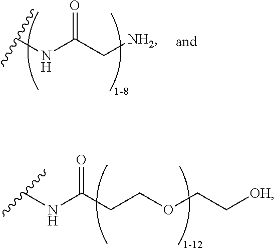

and
m is an integer from 1 to 4
or a pharmaceutically acceptable salt thereof.

The present invention also provides an ATDC having a structure of Formula (I):

BA-L-P (l),

wherein:
BA is an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
L is a non-cleavable linker;
optionally, wherein the heavy chain immunoglobulin of the BA does not comprise the C-terminal lysine or lysine and glycine;
P is a payload having the structure selected from the group consisting of (SEQ ID NOS 451-452, respectively, in order of appearance):

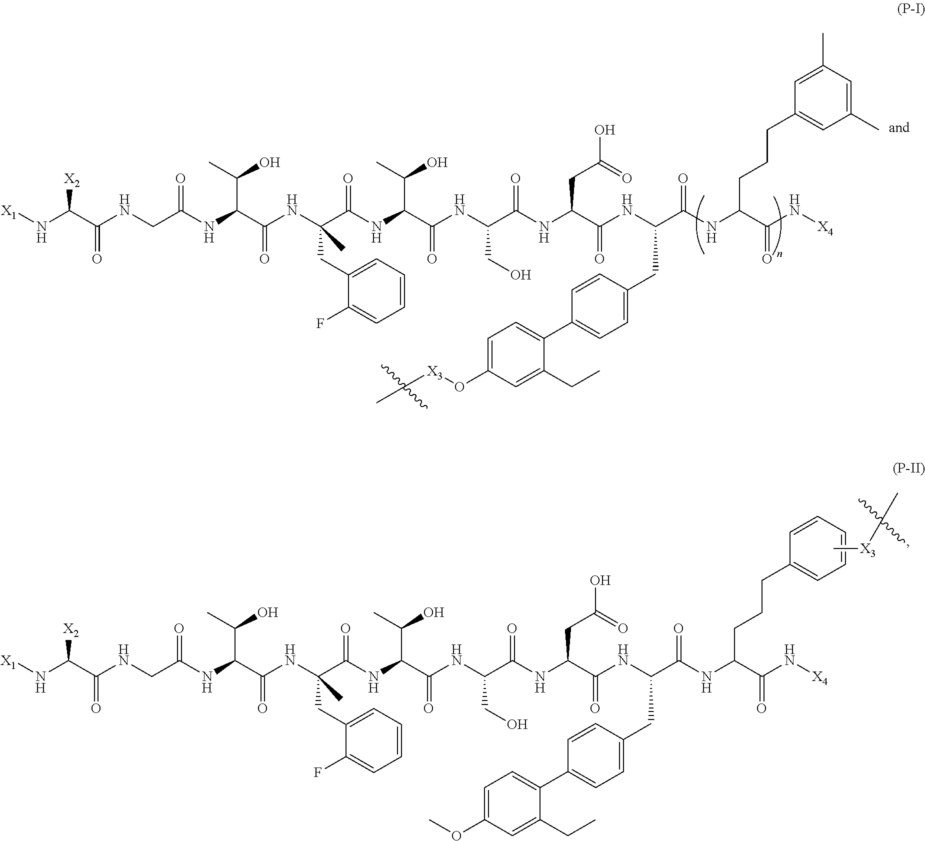

wherein

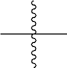

is the point of attachment of the payload P to L;
X₁ is selected from H;

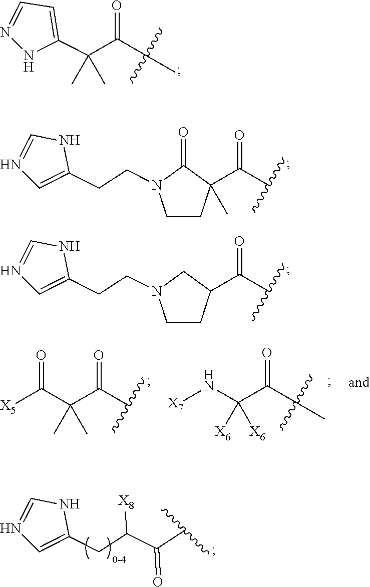

X₂ is selected from

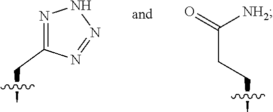

X₃ is selected from —(CH₂)₂₋₆—NH— and —(CH₂)₂₋₆-Tr-, where Tr is a triazole moiety;
n is 0 or 1;
X₄ is selected from H and phenyl;
X₅ is selected from —OH, —NH₂, —NH—OH, and

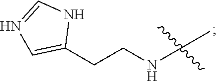 [SMILES: *NCCc1cnc[nH]1]

X₆ is independently at each occurrence selected from H, —OH, —CH₃, and —CH₂OH;
X₇ is selected from H,

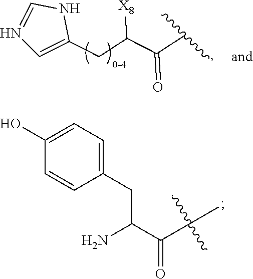

X₈ is selected from H, —OH, —NH₂, and

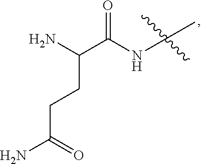 [SMILES: *NC(=O)C(N)CCC(N)=O]

or a pharmaceutically acceptable salt thereof.

In some embodiments of the invention, the BA of the ATDC is an antibody that binds specifically to GLP1R which is antibody 5A10, 9A10, AB9433-1, h38C2, PA5-111834, NLS1205, MAB2814, EPR21819, or glutazumab; or an antigen binding fragment thereof.

In some embodiments, the linker L is attached to one or both heavy chains of the BA. In some embodiments, the linker L is attached to one or both heavy chain variable domains of the BA. In some embodiments, the linker L is attached to one or both light chains of the BA. In some embodiments, the linker L is attached to one or both light chain variable domains of the BA.

In some embodiments of the invention, the linker L is attached to BA via a glutamine residue on the BA. In some embodiments, the glutamine residue is introduced to the N-terminus of one or both heavy chains of the BA. In some embodiments of the invention, the glutamine residue is introduced to the N-terminus of one or both light chains of the BA. In some embodiments, of the invention the glutamine residue is naturally present in a CH2 or CH3 domain of the BA. In some embodiments of the invention, the glutamine residue is introduced to the BA by modifying one or more amino acids. In some embodiments of the invention, the glutamine residue is Q295 or N297Q.

In some embodiments of the invention, the linker L is attached to BA via a lysine residue.

In some embodiments, the heavy chain immunoglobulin of the BA does not comprise the C-terminal lysine or lysine and glycine. In some embodiments, the heavy chain immunoglobulin of the BA does not comprise a C-terminal lysine. In some embodiments, the heavy chain immunoglobulin does not comprise C-terminal lysine and glycine. The heavy chain immunoglobulin that does not comprise C-terminal lysine and glycine also means the heavy chain immunoglobulin that does not comprise lysine and glycine at the C-terminal.

In some embodiments of the invention, the antibody or antigen-binding fragment thereof is aglycosylated. In some embodiments of the invention, the antibody or antigen-binding fragment thereof is deglycosylated. In some embodiments of the invention, the antigen-binding fragment is an Fab fragment.

In one embodiment of the invention, m is 1. In an embodiment, m is an integer from 2 to 4. In one embodiment of the invention, m is 2.

In some embodiments of the invention, more than one L-P is attached to the BA. In some embodiments, two L-Ps are attached to the BA.

In some embodiments, the linker L has the structure of formula (L′):

—La—Y-Lp- (L′),
wherein La is a first linker covalently attached to the BA;
Y is a group comprising a triazole, and
Lp is absent or a second linker covalently attached to the P, wherein when Lp is absent, Y is also absent.

In some embodiments, Y has a structure selected from the group consisting of:

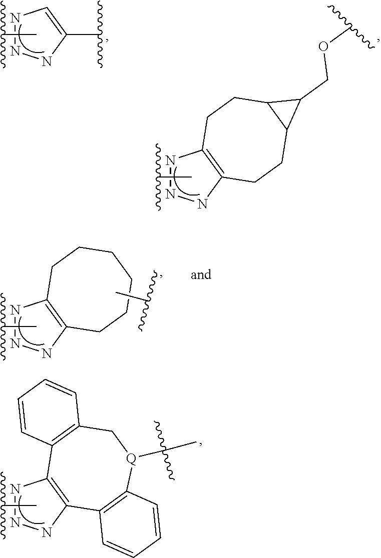

wherein Q is C or N.

In some embodiments of the invention, Lp comprises a polyethylene glycol (PEG) segment having 1 to 36 —CH₂CH₂O— (EG) units. In some embodiments of the invention, the PEG segment comprises between 2 and 30 EG units. In some embodiments of the invention, the PEG segment comprises between 4 and 24 EG units. In some embodiments of the invention, the PEG segment comprises 4 EG units, or 8 EG units, or 12 EG units, or 24 EG units. In some embodiments of the invention, the PEG segment comprises 4 EG units. In some embodiments, the PEG segment comprises 8 EG units.

In some embodiments, Y-Lp has a structure selected from the group consisting of:

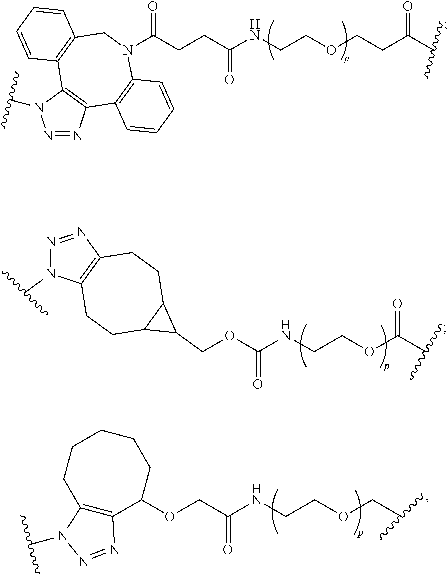

or a triazole regioisomer thereof, wherein p is an integer from 1 to 36.

In some embodiments of the invention, the Lp comprises one or more amino acids selected from glycine, serine, glutamic acid, alanine, valine, and proline and combinations thereof. In some embodiments, the Lp comprises 1 to 10 glycines. In some embodiments, the Lp comprises 1 to 6 serines. In some embodiments of the invention, the Lp comprises 1 to 10 glycines and 1 to 6 serines. In some embodiments of the invention, the Lp comprises 4 glycines and 1 serine. In some embodiments of the invention, the Lp is selected from the group consisting of Gly-Gly-Gly-Gly-Ser (G₄S) (SEQ ID NO: 1), Ser-Gly-Gly-Gly-Gly (SG₄) (SEQ ID NO: 2), and Gly-Gly-Gly-Gly-Ser-Gly-Gly-Gly-Gly-Ser (G₄S-G₄S) (SEQ ID NO: 3).

In some embodiments of the invention, the Lp comprises a combination of a PEG segment having 1 to 36 EG units and one or more amino acids selected from glycine, serine, glutamic acid, alanine, valine, and proline and combinations thereof. In some embodiments of the invention, the serine residue comprises a carbohydrate group. In some embodiments of the invention, the serine residue comprises a glucose group.

In some embodiments of the invention, Lp has a structure selected from the group consisting of (SEQ ID NOS 567-568, respectively, in order of appearance):

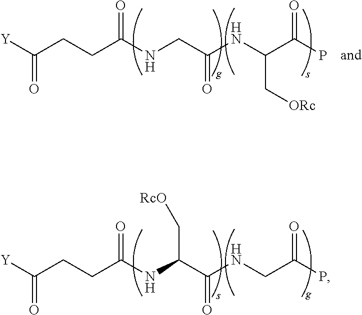

wherein Y is the group comprising a triazole and P is the payload, and wherein Rc is selected from H and glucose, g is an integer from 1 to 10 and s is an integer from 0 to 4.

In some embodiments, Y-Lp has a structure selected from the group consisting of (SEQ ID NOS 453-458, respectively, in order of appearance):

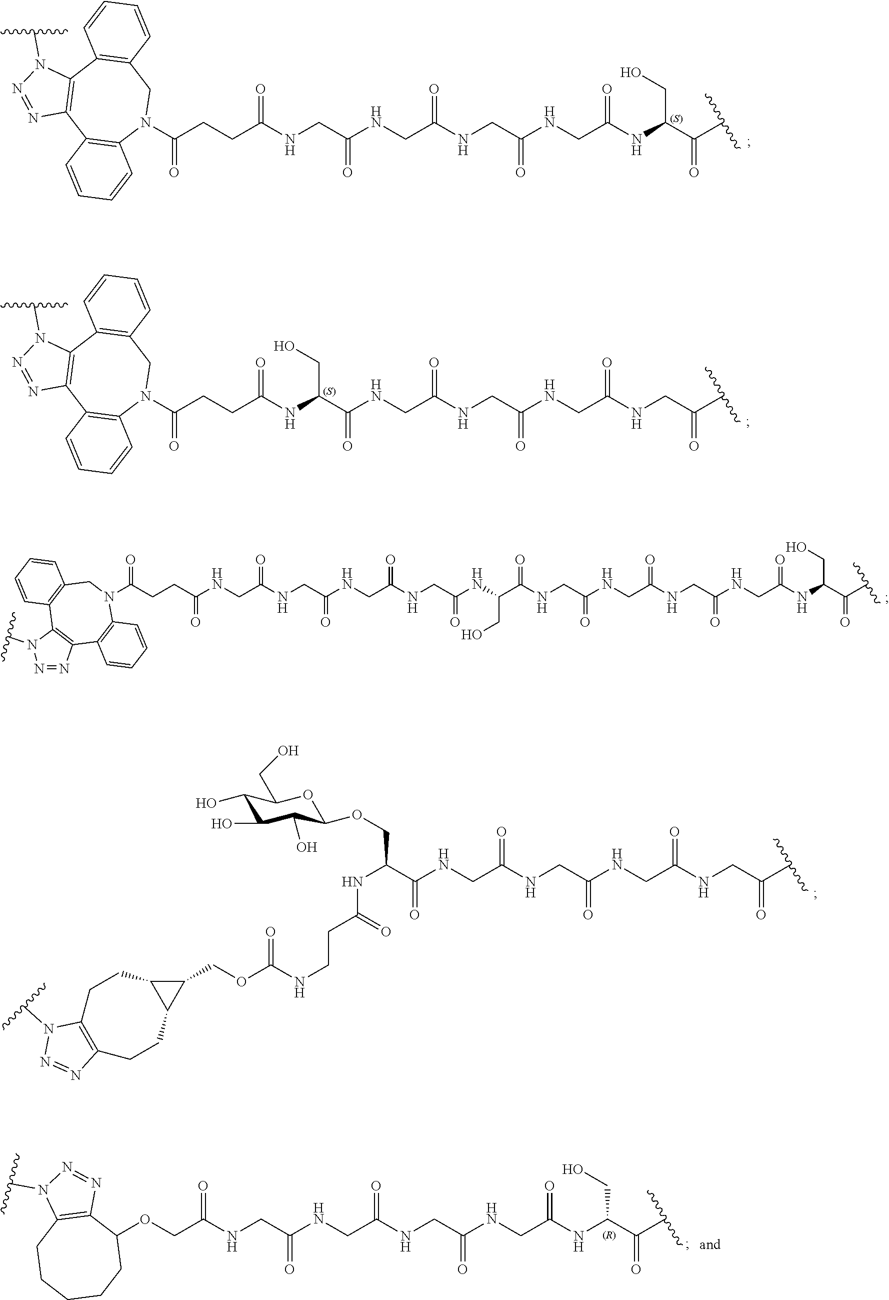

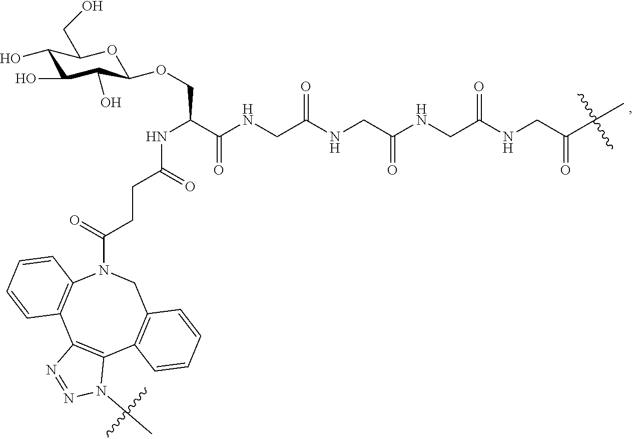

or a triazole regioisomer thereof.

In some embodiments of the invention, La comprises a polyethylene glycol (PEG) segment having 1 to 36 —CH₂CH₂O— (EG) units. In some embodiments of the invention, the PEG segment comprises 4 EG units, or 8 EG units, or 12 EG units, or 24 EG units. In some embodiments, the PEG segment comprises 8 EG units. In some embodiments of the invention, La has a structure selected from the group consisting of

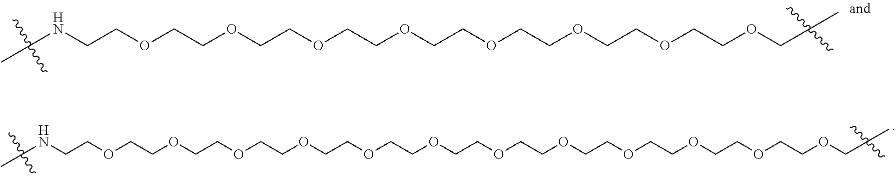 [SMILES: *COCCOCCOCCOCCOCCOCCOCCOCCN* ; *COCCOCCOCCOCCOCCOCCOCCOCCOCCOCCOCCOCCN*]

In some embodiments of the invention, La comprises one or more amino acids selected from glycine, threonine, serine, glutamine, glutamic acid, alanine, valine, leucine, and proline and combinations thereof. In some embodiments of the invention, La comprises 1 to 10 glycines and 1 to 6 serines. In some embodiments of the invention, La comprises 4 glycines and 1 serine. In some embodiments of the invention, La is selected from the group consisting of Gly-Gly-Gly-Gly-Ser (G₄S) (SEQ ID NO: 1), Ser-Gly-Gly-Gly-Gly (SG₄) (SEQ ID NO: 2), Gly-Gly-Ser-Gly-Gly-Ser-Gly-Gly (G₂S-G₂S-G₂) (SEQ ID NO: 438), and Gly-Gly-Gly-Gly-Ser-Gly-Gly-Gly-Gly (G₄S-G₄) (SEQ ID NO: 419).

In some embodiments of the invention, La comprises a combination of a PEG segment having 1 to 36 EG units and one or more amino acids selected from glycine, threonine, serine, glutamine, glutamic acid, alanine, valine, leucine, and proline and combinations thereof. In some embodiments of the invention, La is selected from the group consisting of (SEQ ID NOS 459-460, respectively, in order of appearance):

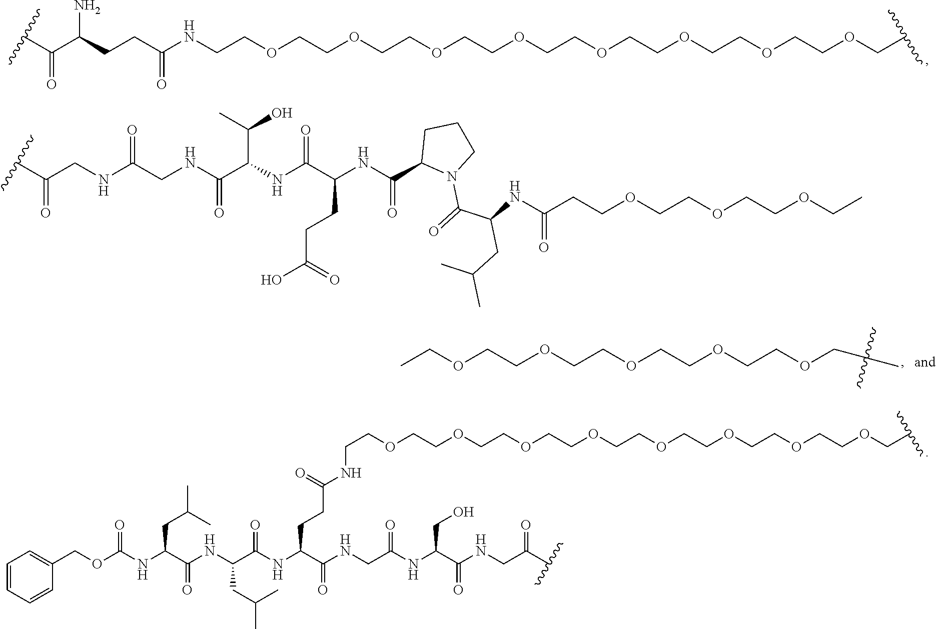

In some embodiments of the invention, La comprises a —(CH₂)₂₋₂₄— chain. In some embodiments, In some embodiments, La comprises a combination of a —(CH₂)₂₋₂₄— chain, a PEG segment having 1 to 36 EG units and one or more amino acids selected from glycine, threonine, serine, glutamine, glutamic acid, alanine, valine, leucine, and proline and combinations thereof. La is selected from the group consisting of (SEQ ID NOS 461-462, respectively, in order of appearance):

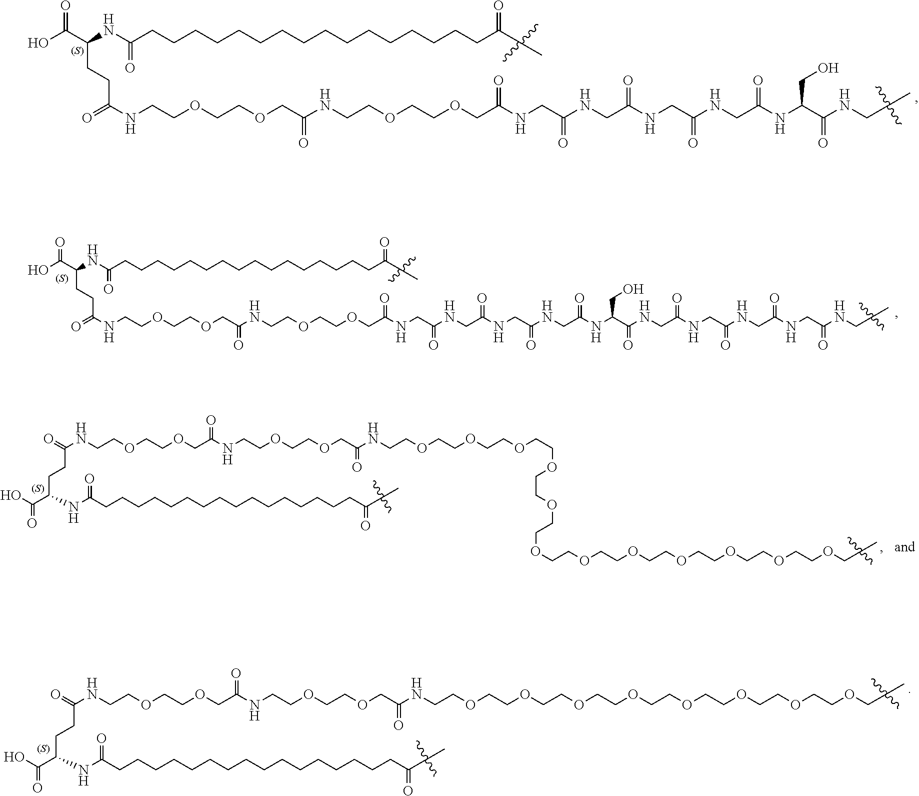

In various embodiments of the invention, P has the structure disclosed as SEQ ID NO:463:

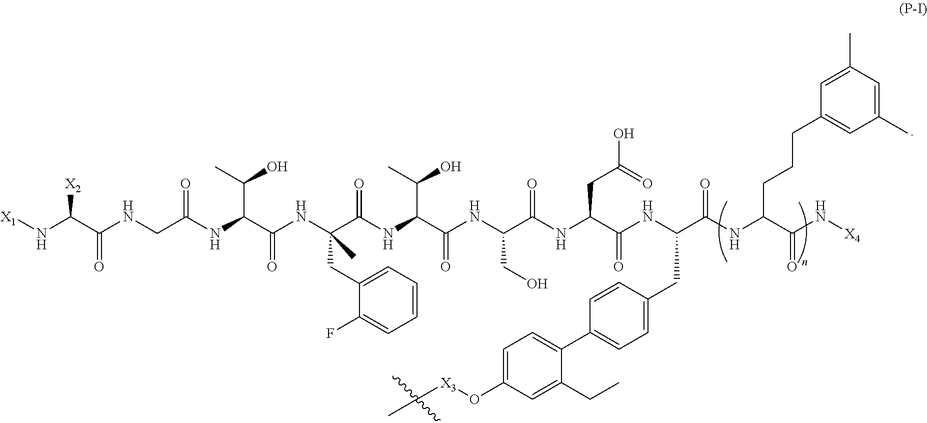

In some embodiments of the invention, X₁ is H; X₂ is

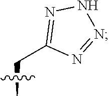

X₃ is selected from —(CH₂)₂₋₆—NH— and —(CH₂)₂₋₆-Tr-, where Tr is a triazole moiety; n is 1, and X₄ is H.

In some embodiments of the invention, X₁ is

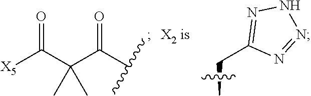

X₃ is —(CH₂)₂₋₆—NH—; n is 1; X₄ is H, and X₅ is selected from —OH, —NH₂, —NH—OH, and

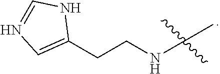 [SMILES: *NCCc1cnc[nH]1]

In some embodiments of the invention, X₁ is

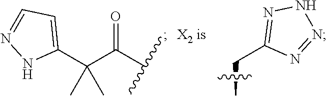

X₃ is —(CH₂)₂₋₆—NH—; n is 1, and X₄ is H.

In some embodiments of the invention, X₁ is

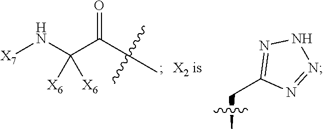

X₃ is —(CH₂)₂₋₆—NH—; n is 1; X₄ is H; X₆ is independently at each occurrence selected from H and —CH₂OH, and X₇ is H.

In some embodiments of the invention, X₁ is

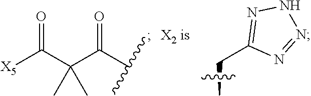

X₃ is —(CH₂)₂₋₆-Tr-, where Tr is a triazole moiety; n is 1; X₄ is H, and X₅ is

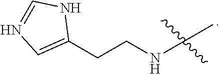 [SMILES: *NCCc1cnc[nH]1]

In some embodiments of the invention, X₁ is

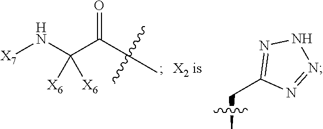

X₃ is —(CH₂)₂₋₆—NH—; n is 1; X₄ is H; X₆ is independently at each occurrence selected from H and —CH₃; X₇ is

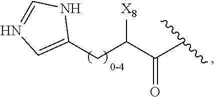

and X₈ is —NH₂.

In some embodiments of the invention, X₁ is

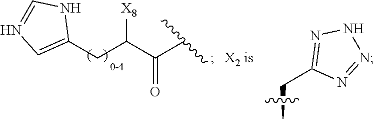

X₃ is —(CH₂)₂₋₆—NH—; n is 1; X₄ is H, and X₈ is H.

In some embodiments of the invention, X₁ is

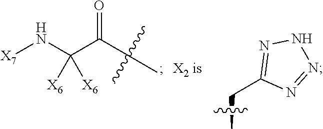

X₃ is —(CH₂)₂₋₆—NH—; n is 1; X₄ is H; X₆ is H at each occurrence; X₇ is

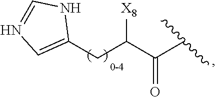

### and X₈ is H.

In some embodiments of the invention, X₁ is

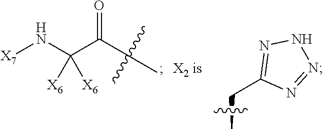

X₃ is —(CH₂)₂₋₆—NH—; n is 1; X₄ is H; X₆ is independently at each occurrence selected from H and —CH₃; X₇ is

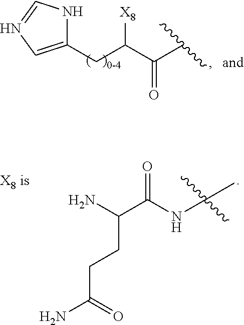

In some embodiments of the invention, X₁ is

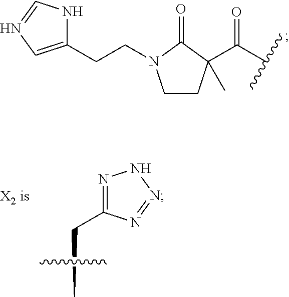

X₃ is —(CH₂)₂₋₆—NH—; n is 1, and X₄ is H.

In some embodiments of the invention, X₁ is

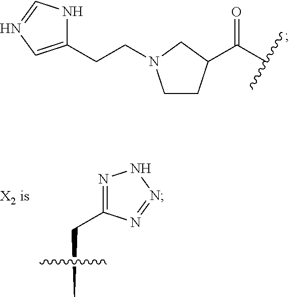

X₃ is —(CH₂)₂₋₆—NH—; n is 1, and X₄ is H.

In some embodiments of the invention, X₁ is

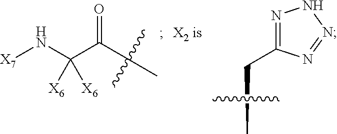

X₃ is —(CH₂)₂₋₆—NH—; n is 1; X₄ is H; X₆ is independently at each occurrence selected from H and —CH₃, and X₇ is

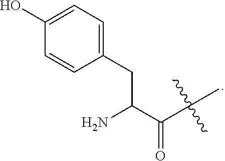 [SMILES: *C(=O)C(N)Cc1ccc(O)cc1]

In some embodiments of the invention, X₁ is H; X₂ is

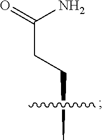

X₃ is —(CH₂)₂₋₆—NH—; n is 1, and X₄ is H.

In some embodiments of the invention, X₁ is

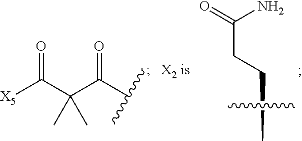

X₃ is —(CH₂)₂₋₆—NH—; n is 1; X₄ is H, and X₅ is

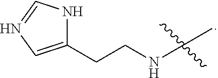 [SMILES: *NCCc1cnc[nH]1]

In some embodiments of the invention, X₁ is

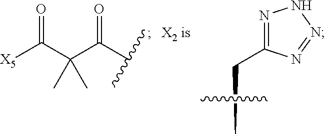

X₃ is —(CH₂)₂₋₆—NH—; n is 0; X₄ is phenyl, and X₅ is

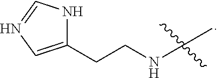 [SMILES: *NCCc1cnc[nH]1]

In some embodiments of the invention, X₁ is

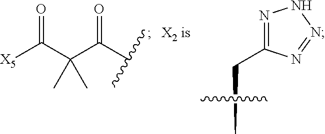

X₃ is —(CH₂)₂₋₆—NH—; n is 1; X₄ is phenyl, and X₅ is

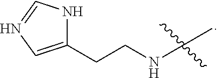 [SMILES: *NCCc1cnc[nH]1]

In various embodiments of the invention, P has the structure disclosed as SEQ ID NO: 464:

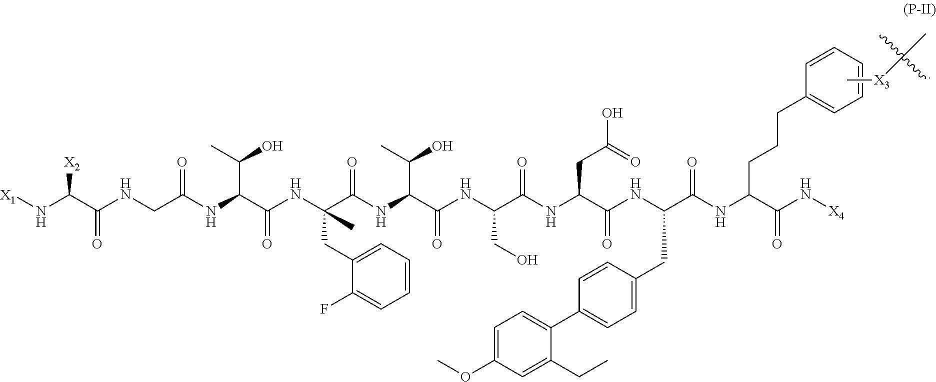

In some embodiments of the invention, X₁ is

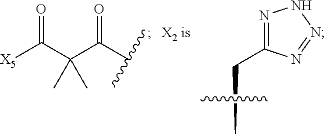

X₃ is —(CH₂)₂₋₆—NH—; X₄ is H, and X₅ is

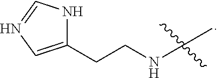 [SMILES: *NCCc1cnc[nH]1]

In various embodiments of the of the invention, P has the structure selected from the group consisting of (SEQ ID NOS 465, 576, 466-495, 610, 496-497, 611, 498-505, respectively, in order of appearance):

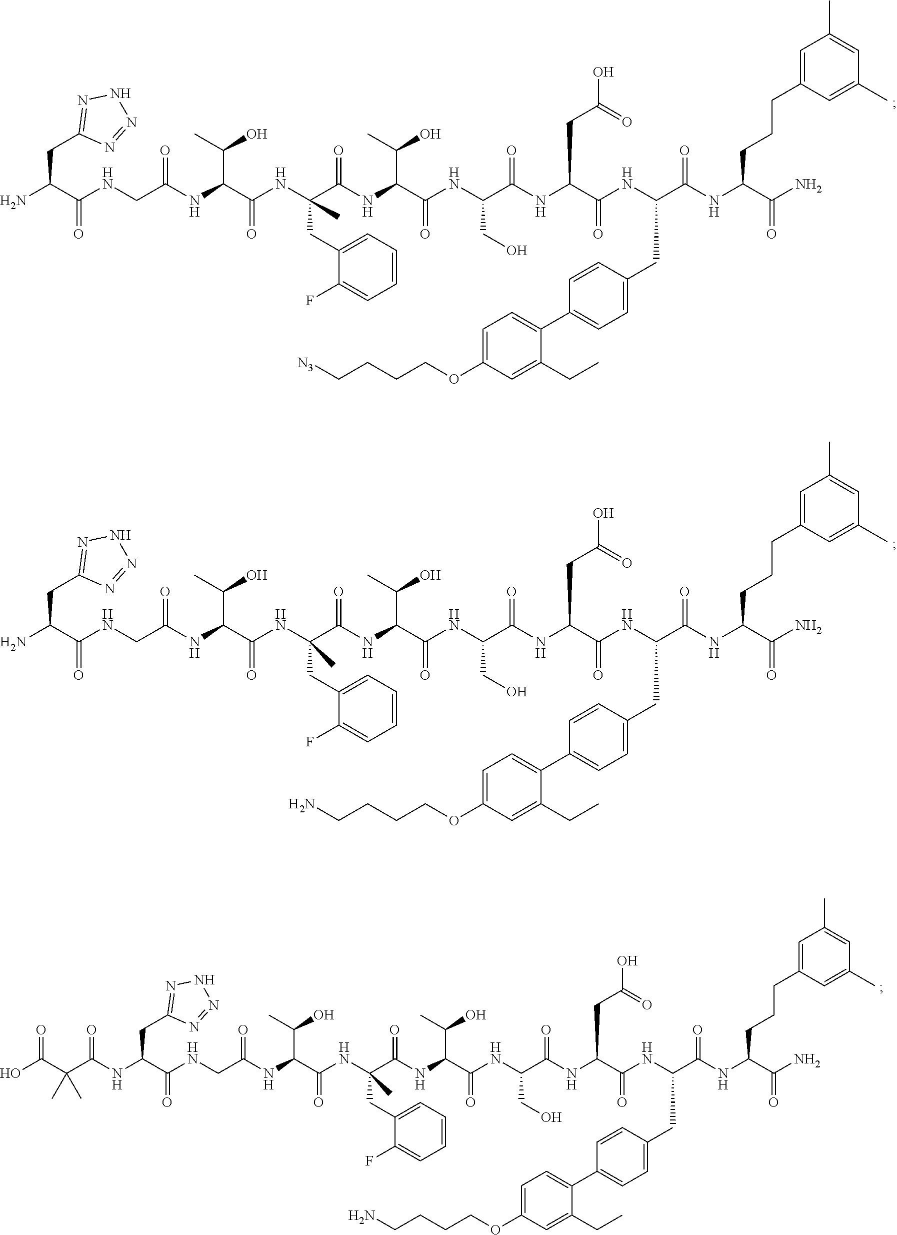

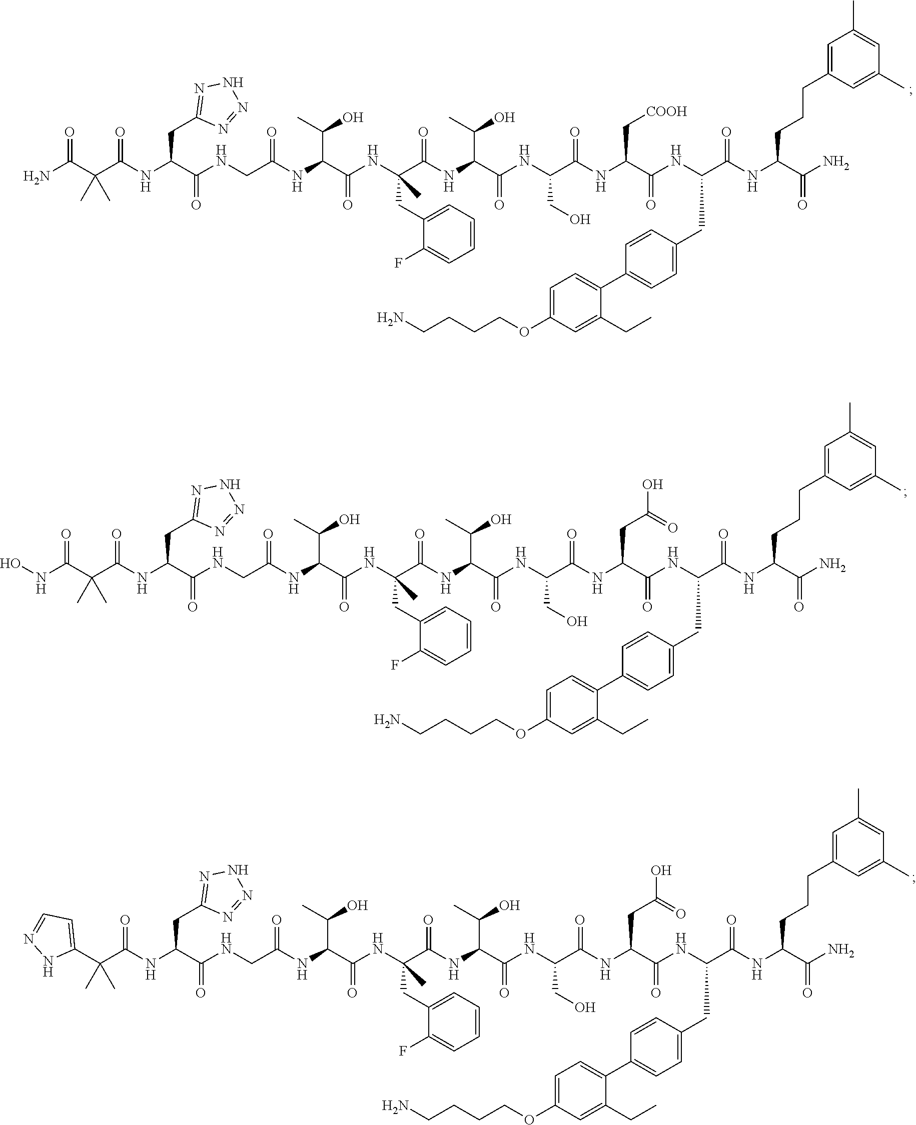

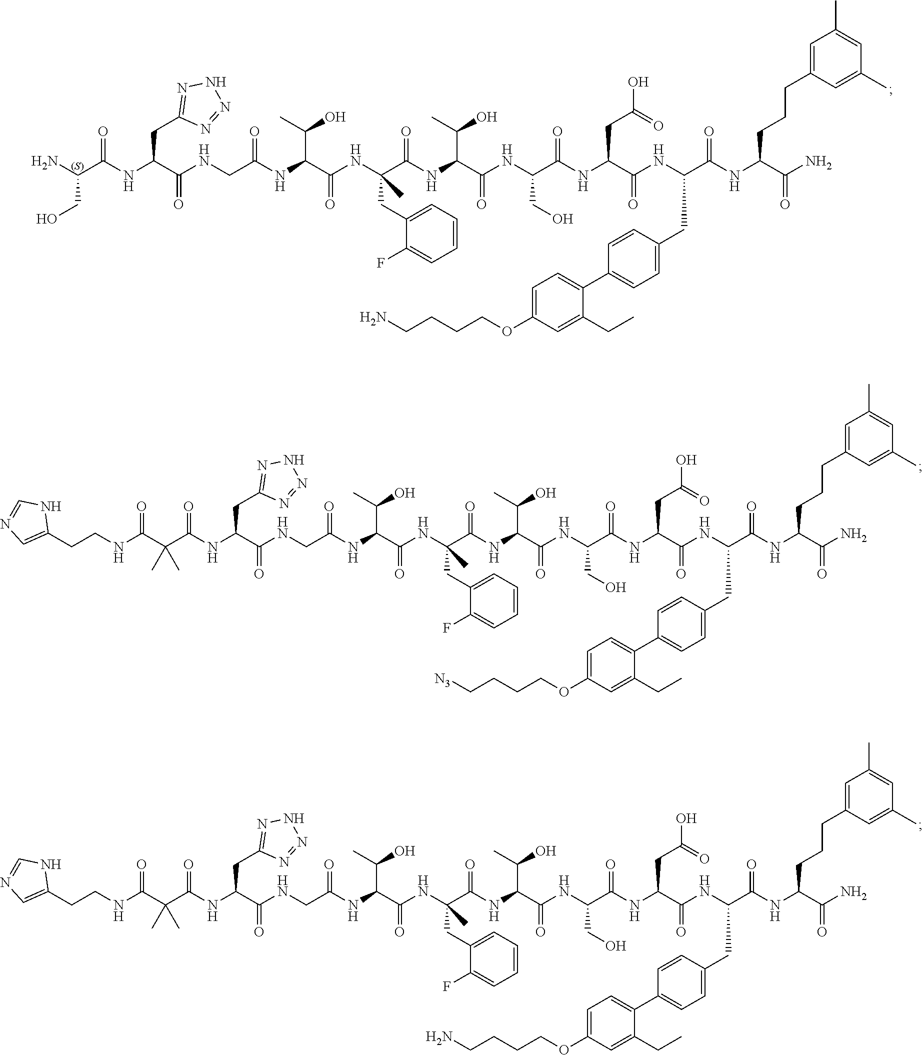

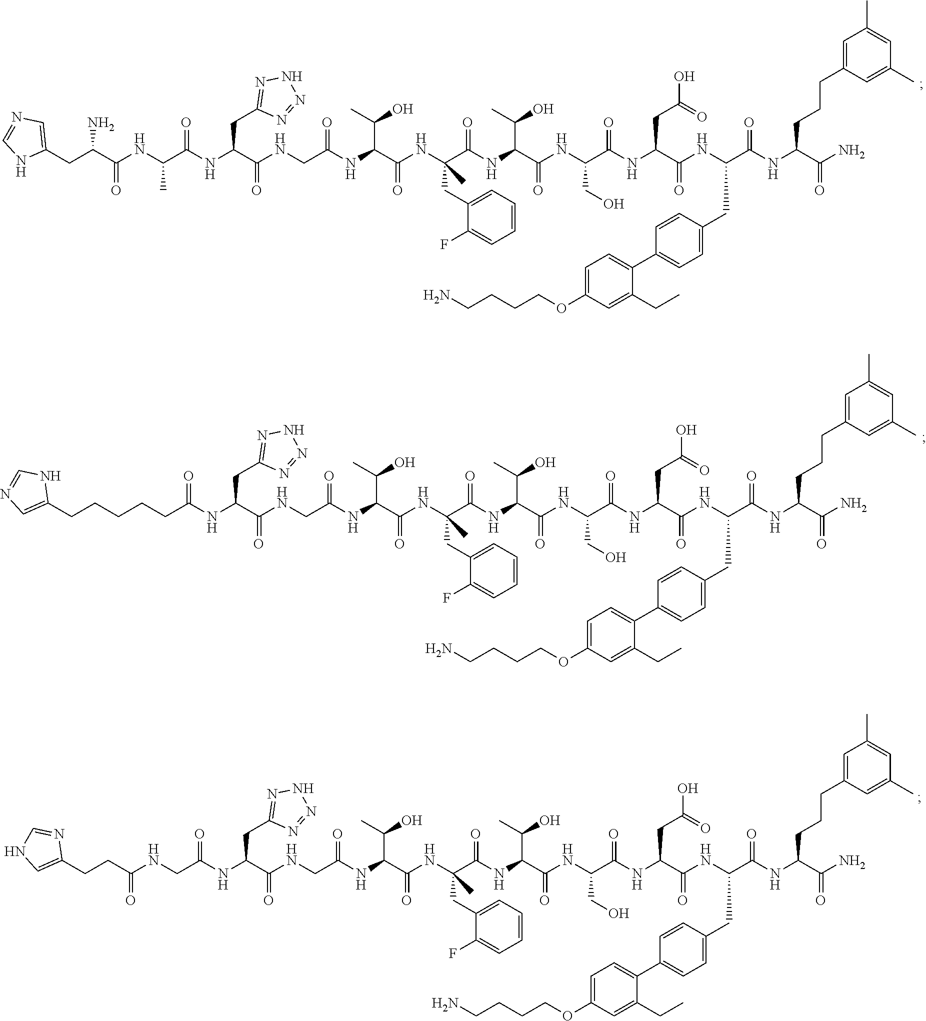

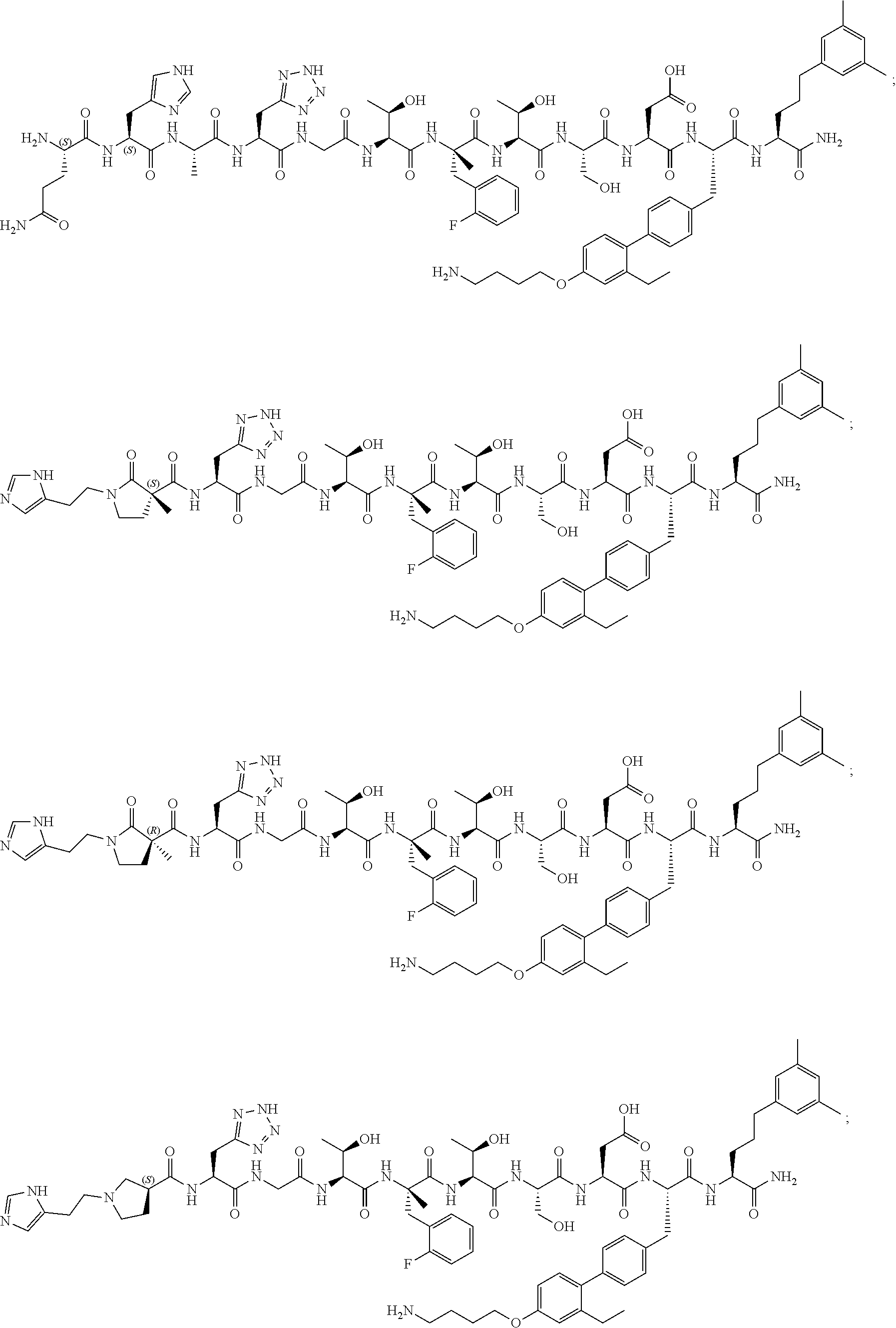

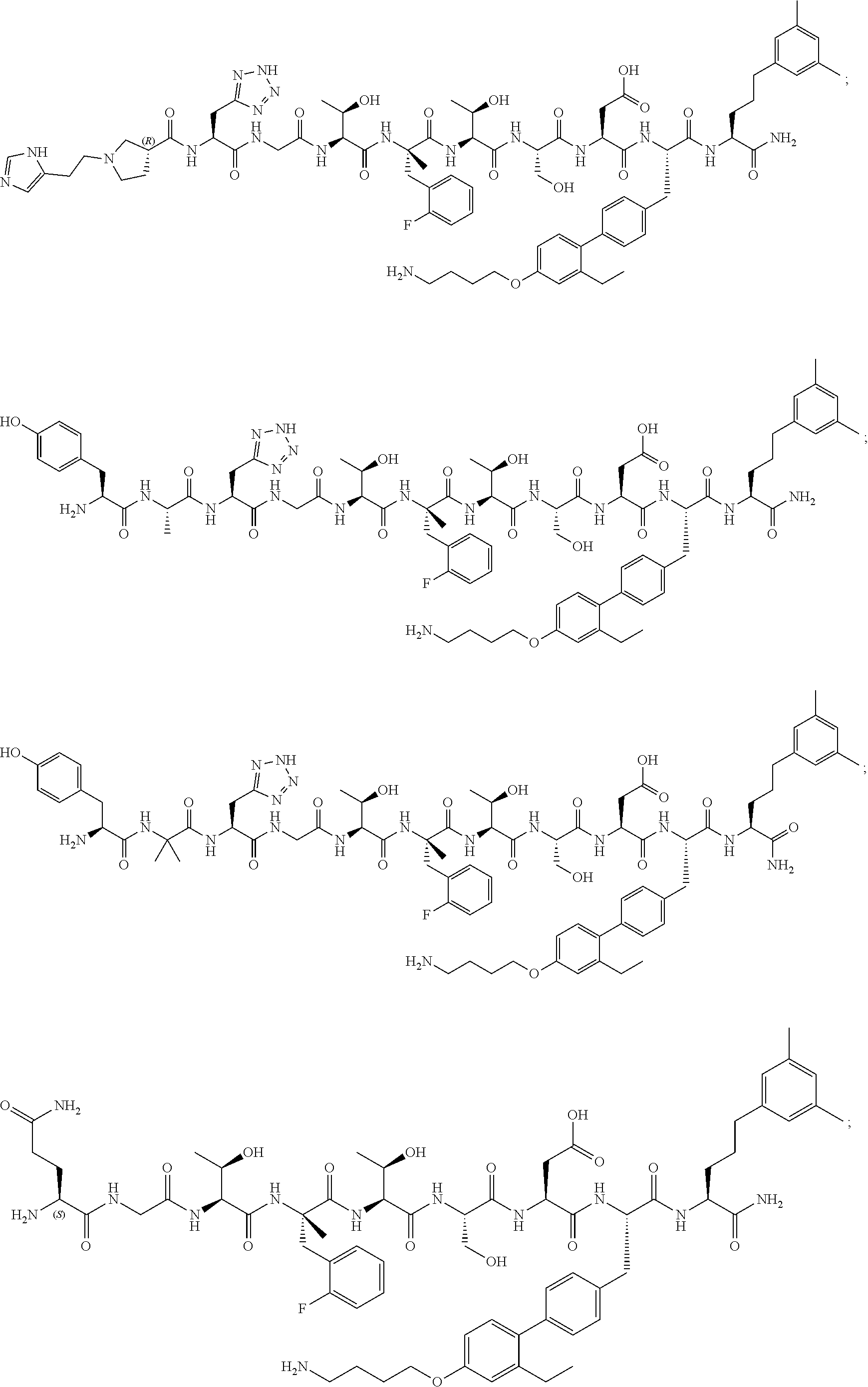

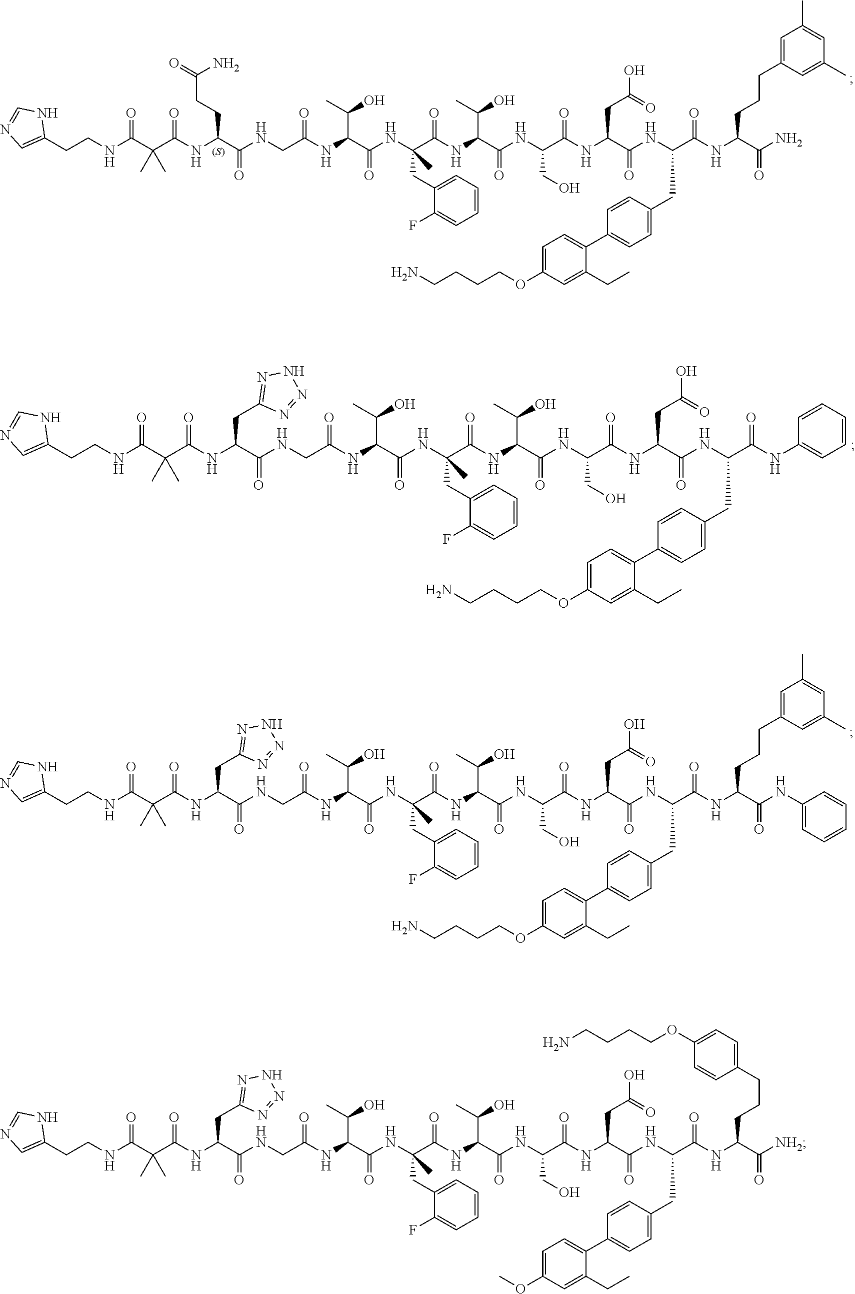

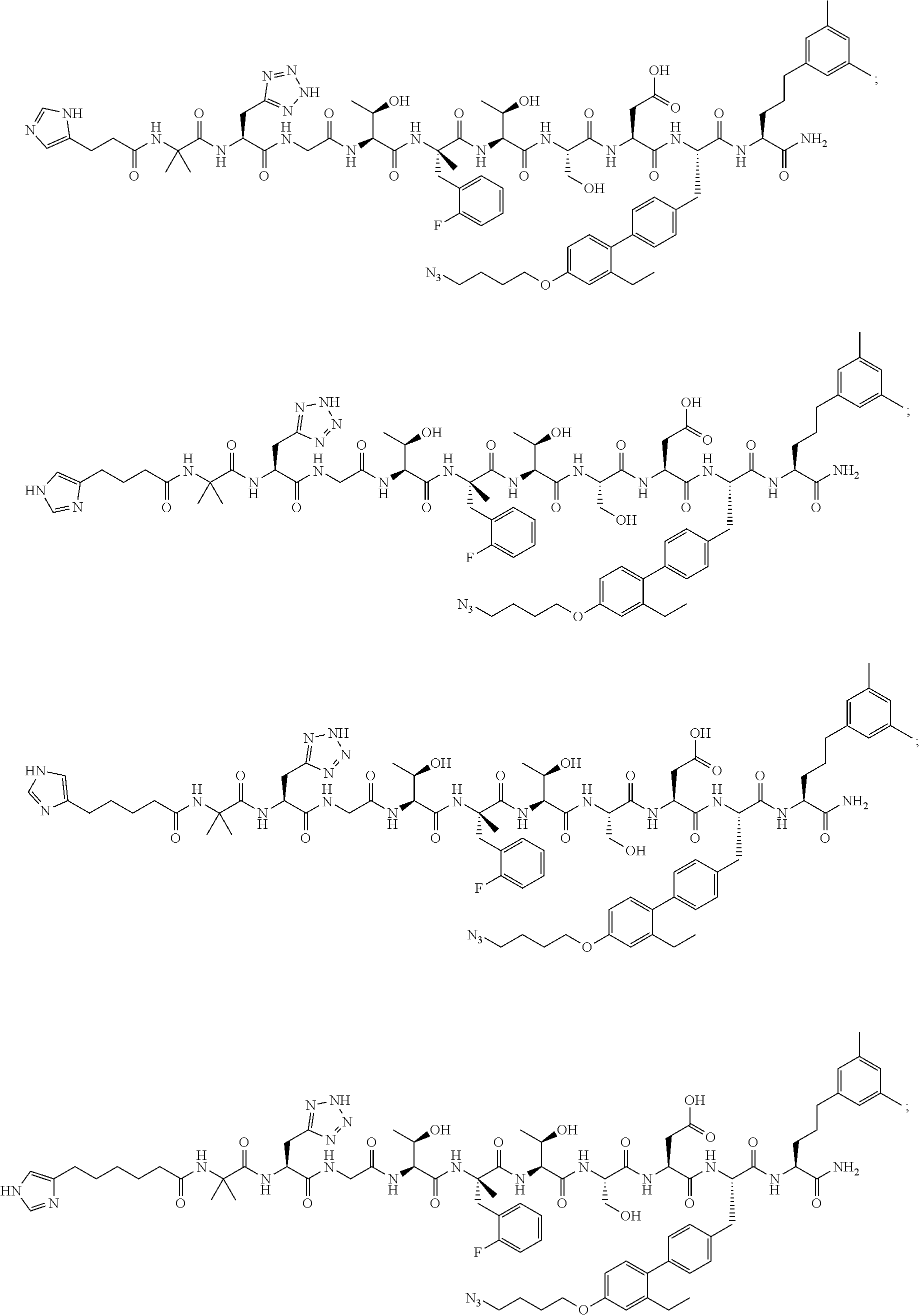

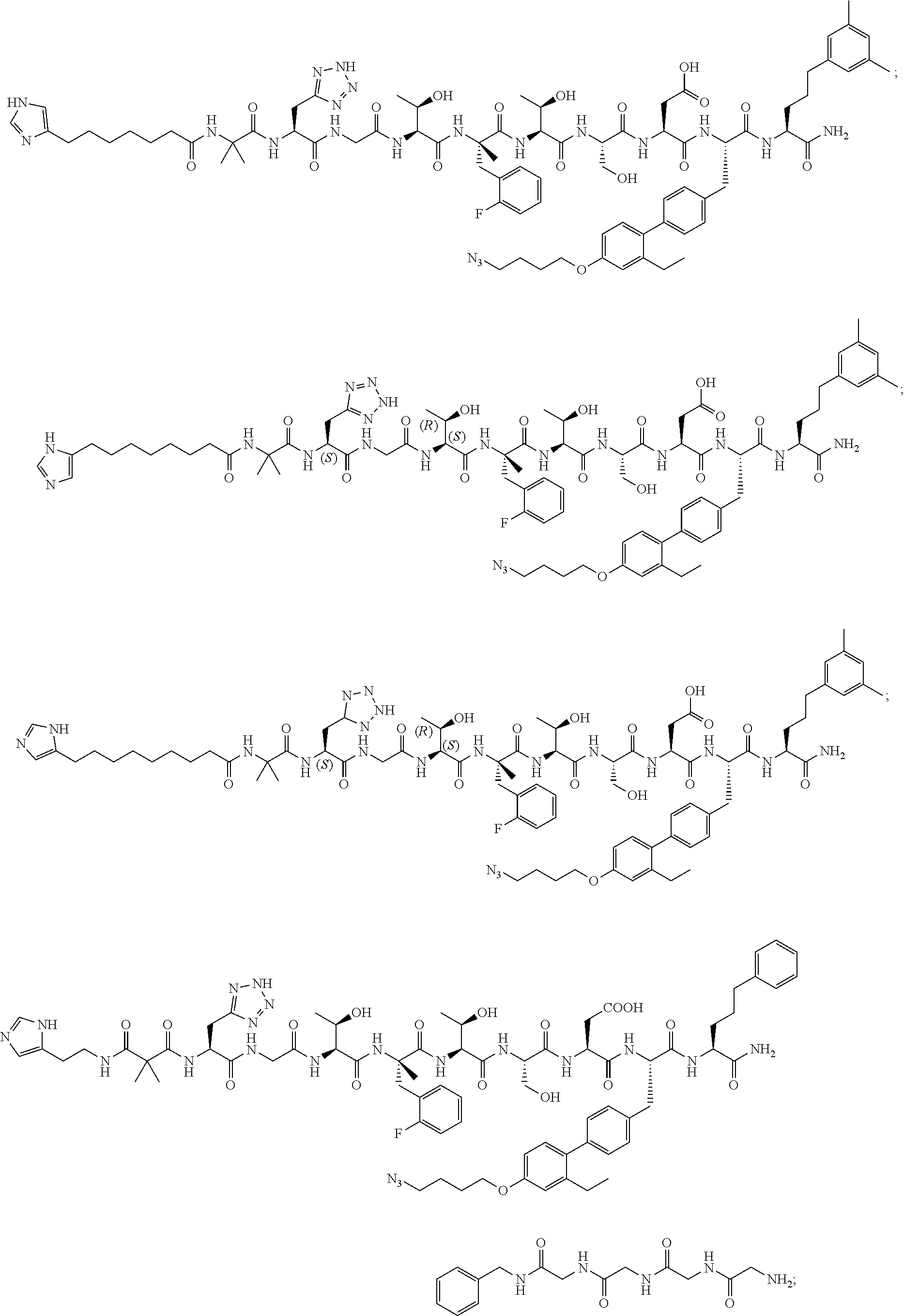

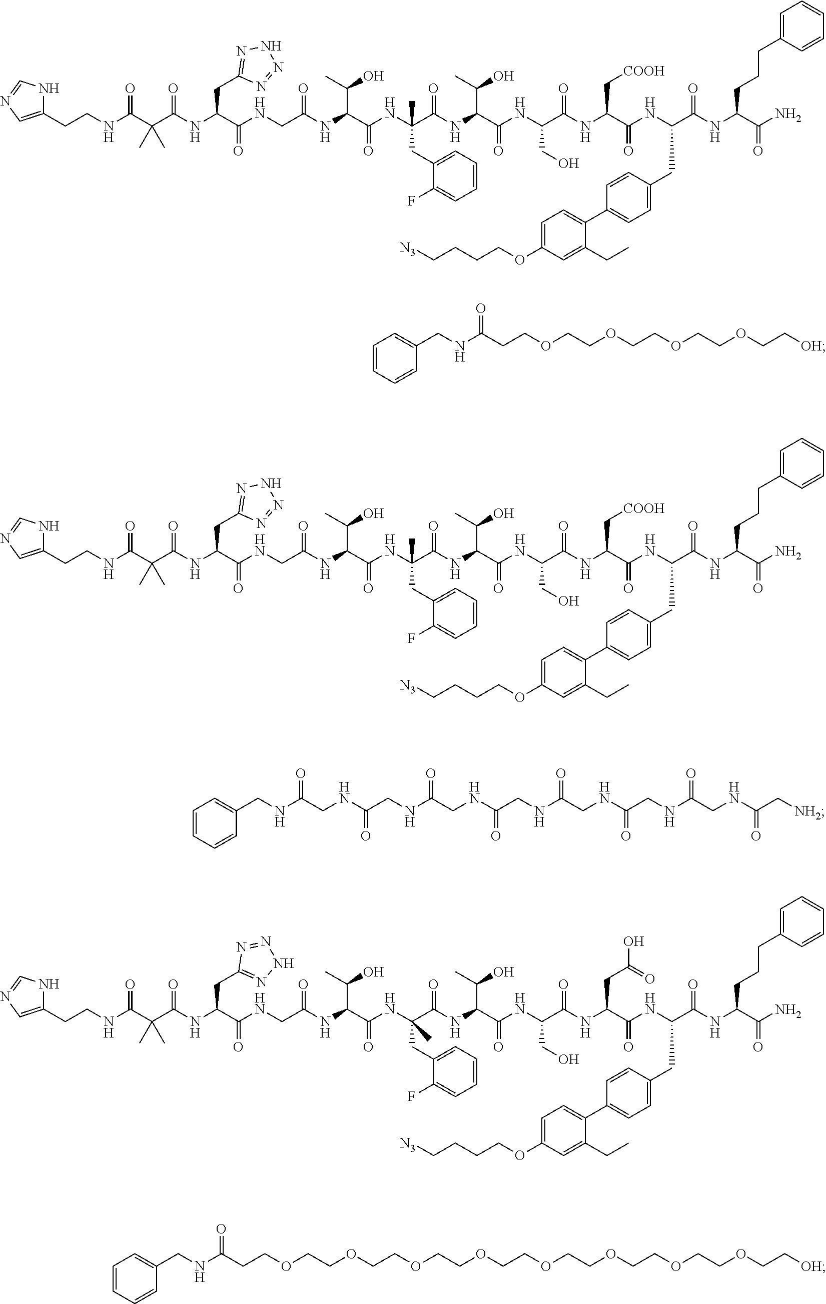

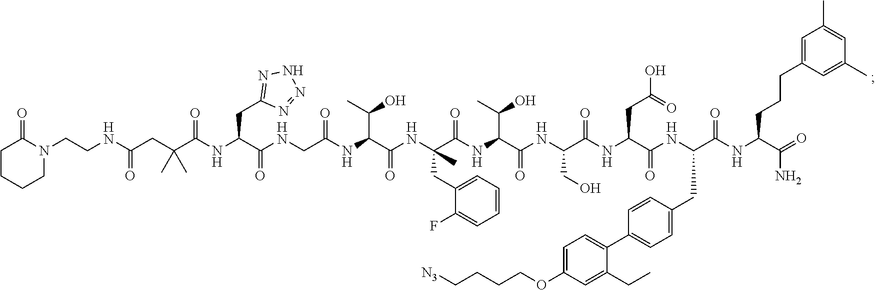 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)CC(=O)NCCN2CCCCC2=O)[C@@H](C)O)[C@@H](C)O)C(=O)N[C@@H](CCCc2cc(C)cc(C)c2)C(N)=O)cc1]

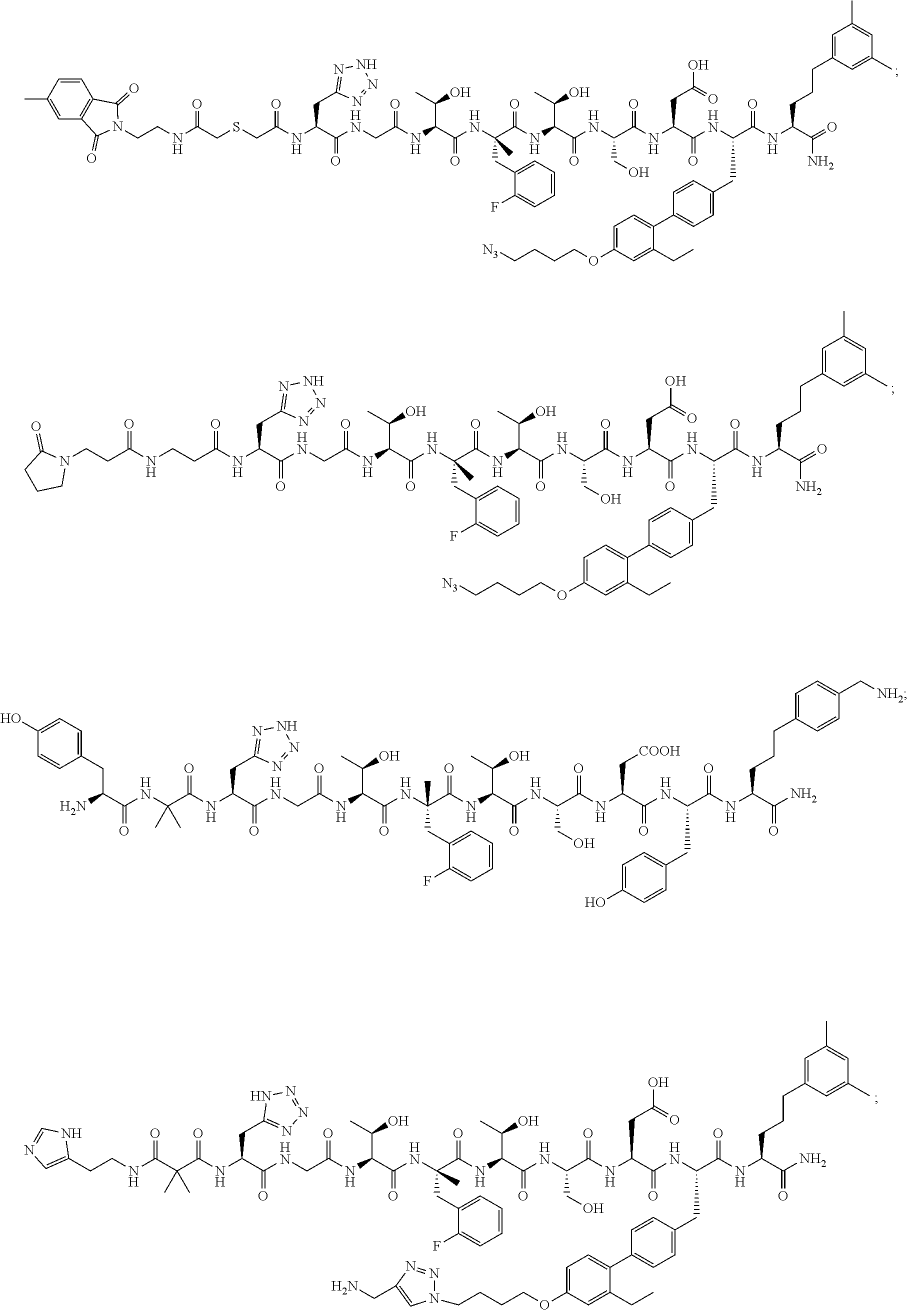

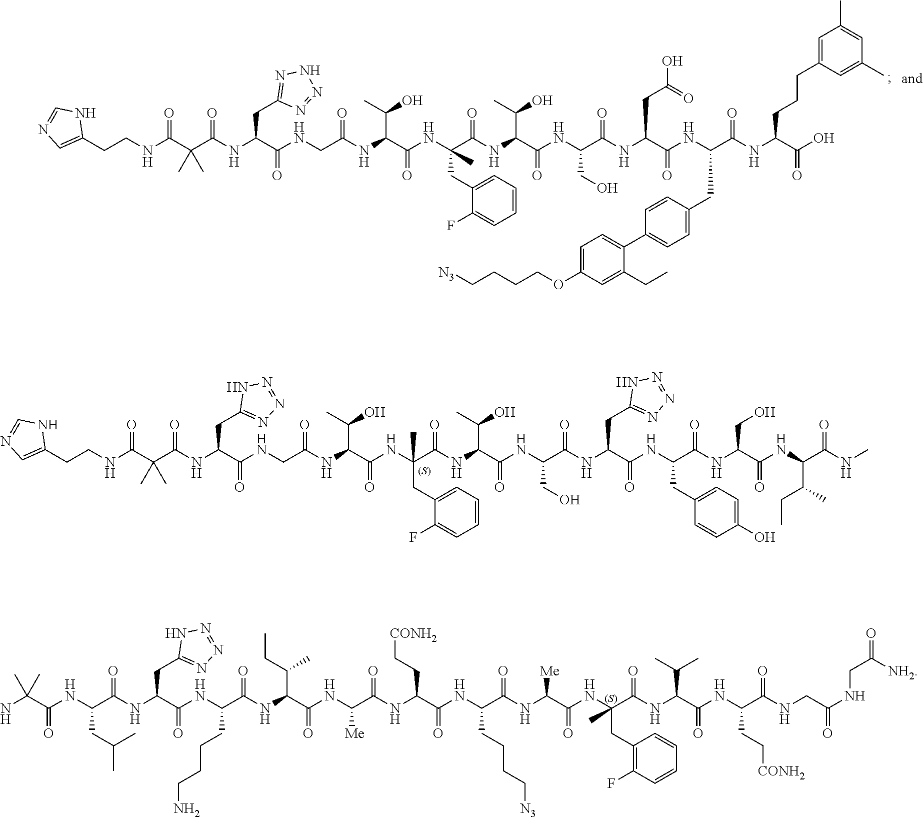

In various embodiments of the invention, the ATDC of the present invention has a half life of longer than 7 days in plasma.

In various embodiments of the invention, the ATDC of the present invention does not bind to G protein-coupled receptors (GPCRs) other than GLP1R.

In another aspect of the invention, provided herein is a pharmaceutical composition comprising the antibody or antigen-binding fragment thereof or ATDC, wherein at least about 80% of the antibody or antigen-binding fragment thereof or ATDC does not comprise a C-terminal lysine or lysine and glycine in any of the heavy chains.

In some embodiments, the antibody or antigen-binding fragment thereof or ATDC does not comprise a C-terminal lysine. In some embodiments, the antibody or antigen-binding fragment thereof or ATDC does not comprise C-terminal lysine and glycine.

In an embodiment of the invention, in the pharmaceutical composition comprises the antibody or antigen-binding fragment thereof or ATDC, at least about 90% of the antibody or antigen-binding fragment thereof or ATDC does not comprise a C-terminal lysine or lysine and glycine in any of the heavy chains.

In an embodiment of the invention, in the pharmaceutical composition comprises the antibody or antigen-binding fragment thereof or ATDC, about 90% of the antibody or antigen-binding fragment thereof or ATDC does not comprise a C-terminal lysine or lysine and glycine in any of the heavy chains.

In an embodiment of the invention, in the pharmaceutical composition comprises the antibody or antigen-binding fragment thereof or ATDC, at least about 95% of the antibody or antigen-binding fragment thereof or ATDC does not comprise a C-terminal lysine or lysine and glycine in any of the heavy chains.

In an embodiment of the invention, in the pharmaceutical composition comprises the antibody or antigen-binding fragment thereof or ATDC, at least about 99% of the antibody or antigen-binding fragment thereof or ATDC does not comprise a C-terminal lysine or lysine and glycine in any of the heavy chains.

In an embodiment of the invention, the heavy chain immunoglobulin described above that does not comprise a C-terminal lysine comprises the amino acid sequence set forth in SEQ ID NO: 414, or 416, or a variant thereof.

In an embodiment of the invention, in the pharmaceutical composition comprises the antibody or antigen-binding fragment thereof or ATDC, less than about 20% of the antibody or antigen-binding fragment or ATDC comprises a C-terminal lysine or lysine and glycine in at least one heavy chain.

In an embodiment of the invention, in the pharmaceutical composition comprises the antibody or antigen-binding fragment thereof or ATDC, less than about 10% of the antibody or antigen-binding fragment or ATDC comprises a C-terminal lysine or lysine and glycine in at least one heavy chain.

In an embodiment of the invention, in the pharmaceutical composition comprises the antibody or antigen-binding fragment thereof or ATDC, about 10% of the antibody or antigen-binding fragment or ATDC comprises a C-terminal lysine or lysine and glycine in at least one heavy chain.

In an embodiment of the invention, in the pharmaceutical composition comprises the antibody or antigen-binding fragment thereof or ATDC, less than about 5% of the antibody or antigen-binding fragment or ATDC comprises a C-terminal lysine or lysine and glycine in at least one heavy chain.

In an embodiment of the invention, in the pharmaceutical composition comprises the antibody or antigen-binding fragment thereof or ATDC, less than abouit 1% of the antibody or antigen-binding fragment or ATDC comprises a C-terminal lysine or lysine and glycine in at least one heavy chain.

In an embodiment of the invention, in the pharmaceutical composition comprises the antibody or antigen-binding fragment thereof or ATDC, about 10% of the antibody or antigen-binding fragment or ATDC comprises a C-terminal lysine or lysine and glycine in at least one heavy chain.

In an embodiment of the invention, the at least one heavy chain that comprises a C-terminal lysine or lysine and glycine described above comprises the amino acid sequence set forth in SEQ ID NO: 42; 62; 82; 102; 122; 142; 162; 182; 203; 223; 243; 263; 267; 271; 291; 311; 331; 351; 371; 391; or 411; or a variant thereof.

In an embodiment of the invention, the at least one heavy chain that comprises a C-terminal lysine described above comprises the amino acid sequence set forth in SEQ ID NO: 82.

In another aspect of the invention, provided herein is a pharmaceutical composition comprising the antibody or antigen-binding fragment therof or ATDC and a pharmaceutically acceptable carrier.

In another aspect of the invention, provided herein is a pharmaceutical dosage form comprising an antibody or antigen-binding fragment thereof or ATDC described herein.

In an embodiment of the invention, the antibody or antigen-binding fragment thereof that binds specifically to GLP1R (e.g., BA of an ATDC as set forth herein) comprises: (a) the heavy chain immunoglobulin or variable region thereof comprises an amino acid sequence having at least 90% amino acid sequence identity to the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; and/or (b) the light chain immunoglobulin or variable region thereof comprises an amino acid sequence having at least 90% amino acid sequence identity to the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413. In some embodiments, the heavy chain immunoglobulin does not comprise a C-terminal lysine or lysine and glycine.

In an embodiment of the invention, the antibody or antigen-binding fragment thereof that binds specifically to GLP1R (e.g., BA of an ATDC as set forth herein) comprises: (a) the heavy chain immunoglobulin or variable region thereof comprises the CDR-H1, CDR-H2 and CDR-H3 of a heavy chain immunoglobulin or variable region thereof comprising an amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411, and at least 90% amino acid sequence identity to the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; and/or (b) the light chain immunoglobulin or variable region thereof comprises the CDR-L1, CDR-L2 and CDR-L3 of a light chain immunoglobulin or variable region thereof comprising an amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413, and at least 90% amino acid sequence identity to the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413. In some embodiments, the heavy chain immunoglobulin does not comprise a C-terminal lysine or lysine and glycine.

In an embodiment of the invention, the antibody or antigen-binding fragment thereof that binds specifically to GLP1R (e.g., BA of an ATDC as set forth herein) comprises: the heavy chain immunoglobulin or variable region thereof comprises: (i) a CDR-H1 comprising the amino acid sequence set forth in SEQ ID NO: 28, or a variant thereof; a CDR-H2 comprising the amino acid sequence set forth in SEQ ID NO: 30, or a variant thereof; a CDR-H3 comprising the amino acid sequence set forth in SEQ ID NO: 32, or a variant thereof; (ii) a CDR-H1 comprising the amino acid sequence set forth in SEQ ID NO: 48, or a variant thereof; a CDR-H2 comprising the amino acid sequence set forth in SEQ ID NO: 50, or a variant thereof; a CDR-H3 comprising the amino acid sequence set forth in SEQ ID NO: 52, or a variant thereof; (iii) a CDR-H1 comprising the amino acid sequence set forth in SEQ ID NO: 68, or a variant thereof; a CDR-H2 comprising the amino acid sequence set forth in SEQ ID NO: 70, or a variant thereof; a CDR-H3 comprising the amino acid sequence set forth in SEQ ID NO: 72, or a variant thereof; (iv) a CDR-H1 comprising the amino acid sequence set forth in SEQ ID NO: 88, or a variant thereof; a CDR-H2 comprising the amino acid sequence set forth in SEQ ID NO: 90, or a variant thereof; a CDR-H3 comprising the amino acid sequence set forth in SEQ ID NO: 92, or a variant thereof; (v) a CDR-H1 comprising the amino acid sequence set forth in SEQ ID NO: 108, or a variant thereof; a CDR-H2 comprising the amino acid sequence set forth in SEQ ID NO: 110, or a variant thereof; a CDR-H3 comprising the amino acid sequence set forth in SEQ ID NO: 112, or a variant thereof; (vi) a CDR-H1 comprising the amino acid sequence set forth in SEQ ID NO: 128, or a variant thereof; a CDR-H2 comprising the amino acid sequence set forth in SEQ ID NO: 130, or a variant thereof; a CDR-H3 comprising the amino acid sequence set forth in SEQ ID NO: 132, or a variant thereof; (vii) a CDR-H1 comprising the amino acid sequence set forth in SEQ ID NO: 148, or a variant thereof; a CDR-H2 comprising the amino acid sequence set forth in SEQ ID NO: 150, or a variant thereof; a CDR-H3 comprising the amino acid sequence set forth in SEQ ID NO: 152, or a variant thereof; (viii) a CDR-H1 comprising the amino acid sequence set forth in SEQ ID NO: 168, or a variant thereof; a CDR-H2 comprising the amino acid sequence set forth in SEQ ID NO: 170, or a variant thereof; a CDR-H3 comprising the amino acid sequence set forth in SEQ ID NO: 172, or a variant thereof; (ix) a CDR-H1 comprising the amino acid sequence set forth in SEQ ID NO: 189, or a variant thereof; a CDR-H2 comprising the amino acid sequence set forth in SEQ ID NO: 191, or a variant thereof; a CDR-H3 comprising the amino acid sequence set forth in SEQ ID NO: 193, or a variant thereof; (x) a CDR-H1 comprising the amino acid sequence set forth in SEQ ID NO: 209, or a variant thereof; a CDR-H2 comprising the amino acid sequence set forth in SEQ ID NO: 211, or a variant thereof; a CDR-H3 comprising the amino acid sequence set forth in SEQ ID NO: 213, or a variant thereof; (xi) a CDR-H1 comprising the amino acid sequence set forth in SEQ ID NO: 229, or a variant thereof; a CDR-H2 comprising the amino acid sequence set forth in SEQ ID NO: 231, or a variant thereof; a CDR-H3 comprising the amino acid sequence set forth in SEQ ID NO: 233, or a variant thereof; (xii) a CDR-H1 comprising the amino acid sequence set forth in SEQ ID NO: 249, or a variant thereof; a CDR-H2 comprising the amino acid sequence set forth in SEQ ID NO: 251, or a variant thereof; a CDR-H3 comprising the amino acid sequence set forth in SEQ ID NO: 253, or a variant thereof; (xiii) a CDR-H1 comprising the amino acid sequence set forth in SEQ ID NO: 277, or a variant thereof; a CDR-H2 comprising the amino acid sequence set forth in SEQ ID NO: 279, or a variant thereof; a CDR-H3 comprising the amino acid sequence set forth in SEQ ID NO: 281, or a variant thereof; (xiv) a CDR-H1 comprising the amino acid sequence set forth in SEQ ID NO: 297, or a variant thereof; a CDR-H2 comprising the amino acid sequence set forth in SEQ ID NO: 299, or a variant thereof; a CDR-H3 comprising the amino acid sequence set forth in SEQ ID NO: 301, or a variant thereof; (xv) a CDR-H1 comprising the amino acid sequence set forth in SEQ ID NO: 317, or a variant thereof; a CDR-H2 comprising the amino acid sequence set forth in SEQ ID NO: 319, or a variant thereof; a CDR-H3 comprising the amino acid sequence set forth in SEQ ID NO: 321, or a variant thereof; (xvi) a CDR-H1 comprising the amino acid sequence set forth in SEQ ID NO: 337, or a variant thereof; a CDR-H2 comprising the amino acid sequence set forth in SEQ ID NO: 339, or a variant thereof; a CDR-H3 comprising the amino acid sequence set forth in SEQ ID NO: 341, or a variant thereof; (xvii) a CDR-H1 comprising the amino acid sequence set forth in SEQ ID NO: 357, or a variant thereof; a CDR-H2 comprising the amino acid sequence set forth in SEQ ID NO: 359, or a variant thereof; a CDR-H3 comprising the amino acid sequence set forth in SEQ ID NO: 361, or a variant thereof; and/or (xviii) a CDR-H1 comprising the amino acid sequence set forth in SEQ ID NO: 377, or a variant thereof; a CDR-H2 comprising the amino acid sequence set forth in SEQ ID NO: 379, or a variant thereof; a CDR-H3 comprising the amino acid sequence set forth in SEQ ID NO: 381, or a variant thereof; (xix) a CDR-H1 comprising the amino acid sequence set forth in SEQ ID NO: 397, or a variant thereof; a CDR-H2 comprising the amino acid sequence set forth in SEQ ID NO: 399, or a variant thereof; a CDR-H3 comprising the amino acid sequence set forth in SEQ ID NO: 401, or a variant thereof; and/or the light chain immunoglobulin or variable region thereof comprises: (a) a CDR-L1 comprising the amino acid sequence set forth in SEQ ID NO: 36, or a variant thereof; a CDR-L2 comprising the amino acid sequence GAS, or a variant thereof; a CDR-L3 comprising the amino acid sequence set forth in SEQ ID NO: 40, or a variant thereof; (b) a CDR-L1 comprising the amino acid sequence set forth in SEQ ID NO: 56, or a variant thereof; a CDR-L2 comprising the amino acid sequence AAS, or a variant thereof; a CDR-L3 comprising the amino acid sequence set forth in SEQ ID NO: 60, or a variant thereof; (c) a CDR-L1 comprising the amino acid sequence set forth in SEQ ID NO: 76, or a variant thereof; a CDR-L2 comprising the amino acid sequence AAS, or a variant thereof; a CDR-L3 comprising the amino acid sequence set forth in SEQ ID NO: 80, or a variant thereof; (d) a CDR-L1 comprising the amino acid sequence set forth in SEQ ID NO: 96, or a variant thereof; a CDR-L2 comprising the amino acid sequence KIS, or a variant thereof; a CDR-L3 comprising the amino acid sequence set forth in SEQ ID NO: 100, or a variant thereof; (e) a CDR-L1 comprising the amino acid sequence set forth in SEQ ID NO: 116, or a variant thereof; a CDR-L2 comprising the amino acid sequence AAS, or a variant thereof; a CDR-L3 comprising the amino acid sequence set forth in SEQ ID NO: 120, or a variant thereof; (f) a CDR-L1 comprising the amino acid sequence set forth in SEQ ID NO: 136, or a variant thereof; a CDR-L2 comprising the amino acid sequence GAS, or a variant thereof; a CDR-L3 comprising the amino acid sequence set forth in SEQ ID NO: 140, or a variant thereof; (g) a CDR-L1 comprising the amino acid sequence set forth in SEQ ID NO: 156, or a variant thereof; a CDR-L2 comprising the amino acid sequence AAS, or a variant thereof; a CDR-L3 comprising the amino acid sequence set forth in SEQ ID NO: 160, or a variant thereof; (h) a CDR-L1 comprising the amino acid sequence set forth in SEQ ID NO: 176, or a variant thereof; a CDR-L2 comprising the amino acid sequence AAS, or a variant thereof; a CDR-L3 comprising the amino acid sequence set forth in SEQ ID NO: 180, or a variant thereof; (i) a CDR-L1 comprising the amino acid sequence set forth in SEQ ID NO: 197, or a variant thereof; a CDR-L2 comprising the amino acid sequence AAS, or a variant thereof; a CDR-L3 comprising the amino acid sequence set forth in SEQ ID NO: 201, or a variant thereof; (j) a CDR-L1 comprising the amino acid sequence set forth in SEQ ID NO: 217, or a variant thereof; a CDR-L2 comprising the amino acid sequence KIS, or a variant thereof; a CDR-L3 comprising the amino acid sequence set forth in SEQ ID NO: 221, or a variant thereof; (k) a CDR-L1 comprising the amino acid sequence set forth in SEQ ID NO: 237, or a variant thereof; a CDR-L2 comprising the amino acid sequence AAS, or a variant thereof; a CDR-L3 comprising the amino acid sequence set forth in SEQ ID NO: 241, or a variant thereof; (l) a CDR-L1 comprising the amino acid sequence set forth in SEQ ID NO: 257, or a variant thereof; a CDR-L2 comprising the amino acid sequence AAS, or a variant thereof; a CDR-L3 comprising the amino acid sequence set forth in SEQ ID NO: 261, or a variant thereof; (m) a CDR-L1 comprising the amino acid sequence set forth in SEQ ID NO: 285, or a variant thereof; a CDR-L2 comprising the amino acid sequence AAS, or a variant thereof; a CDR-L3 comprising the amino acid sequence set forth in SEQ ID NO: 289, or a variant thereof; (n) a CDR-L1 comprising the amino acid sequence set forth in SEQ ID NO: 305, or a variant thereof; a CDR-L2 comprising the amino acid sequence AAS, or a variant thereof; a CDR-L3 comprising the amino acid sequence set forth in SEQ ID NO: 309, or a variant thereof; (o) a CDR-L1 comprising the amino acid sequence set forth in SEQ ID NO: 325, or a variant thereof; a CDR-L2 comprising the amino acid sequence AAS, or a variant thereof; a CDR-L3 comprising the amino acid sequence set forth in SEQ ID NO: 329, or a variant thereof; (p) a CDR-L1 comprising the amino acid sequence set forth in SEQ ID NO: 345, or a variant thereof; a CDR-L2 comprising the amino acid sequence GAS, or a variant thereof; a CDR-L3 comprising the amino acid sequence set forth in SEQ ID NO: 349, or a variant thereof; (q) a CDR-L1 comprising the amino acid sequence set forth in SEQ ID NO: 365, or a variant thereof; a CDR-L2 comprising the amino acid sequence GAS, or a variant thereof; a CDR-L3 comprising the amino acid sequence set forth in SEQ ID NO: 369, or a variant thereof; (r) a CDR-L1 comprising the amino acid sequence set forth in SEQ ID NO: 385, or a variant thereof; a CDR-L2 comprising the amino acid sequence GAS, or a variant thereof; a CDR-L3 comprising the amino acid sequence set forth in SEQ ID NO: 389, or a variant thereof; and/or (s) a CDR-L1 comprising the amino acid sequence set forth in SEQ ID NO: 405, or a variant thereof; a CDR-L2 comprising the amino acid sequence GAS, or a variant thereof; a CDR-L3 comprising the amino acid sequence set forth in SEQ ID NO: 409, or a variant thereof.

In an embodiment of the invention, the antibody or antigen-binding fragment thereof that binds specifically to GLP1R (e.g., BA of an ATDC as set forth herein) comprises: (1) the heavy chain immunoglobulin or variable region thereof comprises a CDR-H1 comprising the amino acid sequence set forth in SEQ ID NO: 28, or a variant thereof; a CDR-H2 comprising the amino acid sequence set forth in SEQ ID NO: 30, or a variant thereof; and a CDR-H3 comprising the amino acid sequence set forth in SEQ ID NO: 32; and the light chain immunoglobulin or variable region thereof comprises a CDR-L1 comprising the amino acid sequence set forth in SEQ ID NO: 36, or a variant thereof; a CDR-L2 comprising the amino acid sequence GAS, or a variant thereof; and a CDR-L3 comprising the amino acid sequence set forth in SEQ ID NO: 40, or a variant thereof; (2) the heavy chain immunoglobulin or variable region thereof comprises a CDR-H1 comprising the amino acid sequence set forth in SEQ ID NO: 48, or a variant thereof; a CDR-H2 comprising the amino acid sequence set forth in SEQ ID NO: 50, or a variant thereof; and a CDR-H3 comprising the amino acid sequence set forth in SEQ ID NO: 52; and the light chain immunoglobulin or variable region thereof comprises a CDR-L1 comprising the amino acid sequence set forth in SEQ ID NO: 56, or a variant thereof; a CDR-L2 comprising the amino acid sequence AAS, or a variant thereof; and a CDR-L3 comprising the amino acid sequence set forth in SEQ ID NO: 60, or a variant thereof; (3) the heavy chain immunoglobulin or variable region thereof comprises a CDR-H1 comprising the amino acid sequence set forth in SEQ ID NO: 68, or a variant thereof; a CDR-H2 comprising the amino acid sequence set forth in SEQ ID NO: 70, or a variant thereof; and a CDR-H3 comprising the amino acid sequence set forth in SEQ ID NO: 72; and the light chain immunoglobulin or variable region thereof comprises a CDR-L1 comprising the amino acid sequence set forth in SEQ ID NO: 76, or a variant thereof; a CDR-L2 comprising the amino acid sequence AAS, or a variant thereof; and a CDR-L3 comprising the amino acid sequence set forth in SEQ ID NO: 80, or a variant thereof; (4) the heavy chain immunoglobulin or variable region thereof comprises a CDR-H1 comprising the amino acid sequence set forth in SEQ ID NO: 88, or a variant thereof; a CDR-H2 comprising the amino acid sequence set forth in SEQ ID NO: 90, or a variant thereof; and a CDR-H3 comprising the amino acid sequence set forth in SEQ ID NO: 92; and the light chain immunoglobulin or variable region thereof comprises a CDR-L1 comprising the amino acid sequence set forth in SEQ ID NO: 96, or a variant thereof; a CDR-L2 comprising the amino acid sequence KIS, or a variant thereof; and a CDR-L3 comprising the amino acid sequence set forth in SEQ ID NO: 100, or a variant thereof; (5) the heavy chain immunoglobulin or variable region thereof comprises a CDR-H1 comprising the amino acid sequence set forth in SEQ ID NO: 108, or a variant thereof; a CDR-H2 comprising the amino acid sequence set forth in SEQ ID NO: 110, or a variant thereof; and a CDR-H3 comprising the amino acid sequence set forth in SEQ ID NO: 112; and the light chain immunoglobulin or variable region thereof comprises a CDR-L1 comprising the amino acid sequence set forth in SEQ ID NO: 116, or a variant thereof; a CDR-L2 comprising the amino acid sequence AAS, or a variant thereof; and a CDR-L3 comprising the amino acid sequence set forth in SEQ ID NO: 120, or a variant thereof; (6) the heavy chain immunoglobulin or variable region thereof comprises a CDR-H1 comprising the amino acid sequence set forth in SEQ ID NO: 128, or a variant thereof; a CDR-H2 comprising the amino acid sequence set forth in SEQ ID NO: 130, or a variant thereof; and a CDR-H3 comprising the amino acid sequence set forth in SEQ ID NO: 132; and the light chain immunoglobulin or variable region thereof comprises a CDR-L1 comprising the amino acid sequence set forth in SEQ ID NO: 136, or a variant thereof; a CDR-L2 comprising the amino acid sequence GAS, or a variant thereof; and a CDR-L3 comprising the amino acid sequence set forth in SEQ ID NO: 140, or a variant thereof; (7) the heavy chain immunoglobulin or variable region thereof comprises a CDR-H1 comprising the amino acid sequence set forth in SEQ ID NO: 148, or a variant thereof; a CDR-H2 comprising the amino acid sequence set forth in SEQ ID NO: 150, or a variant thereof; and a CDR-H3 comprising the amino acid sequence set forth in SEQ ID NO: 152; and the light chain immunoglobulin or variable region thereof comprises a CDR-L1 comprising the amino acid sequence set forth in SEQ ID NO: 156, or a variant thereof; a CDR-L2 comprising the amino acid sequence AAS, or a variant thereof; and a CDR-L3 comprising the amino acid sequence set forth in SEQ ID NO: 160, or a variant thereof; (8) the heavy chain immunoglobulin or variable region thereof comprises a CDR-H1 comprising the amino acid sequence set forth in SEQ ID NO: 168, or a variant thereof; a CDR-H2 comprising the amino acid sequence set forth in SEQ ID NO: 170, or a variant thereof; and a CDR-H3 comprising the amino acid sequence set forth in SEQ ID NO: 172; and the light chain immunoglobulin or variable region thereof comprises a CDR-L1 comprising the amino acid sequence set forth in SEQ ID NO: 176, or a variant thereof; a CDR-L2 comprising the amino acid sequence AAS, or a variant thereof; and a CDR-L3 comprising the amino acid sequence set forth in SEQ ID NO: 180, or a variant thereof; (9) the heavy chain immunoglobulin or variable region thereof comprises a CDR-H1 comprising the amino acid sequence set forth in SEQ ID NO: 189, or a variant thereof; a CDR-H2 comprising the amino acid sequence set forth in SEQ ID NO: 191, or a variant thereof; a CDR-H3 comprising the amino acid sequence set forth in SEQ ID NO: 193, or a variant thereof; and the light chain immunoglobulin or variable region thereof comprises a CDR-L1 comprising the amino acid sequence set forth in SEQ ID NO: 197, or a variant thereof; a CDR-L2 comprising the amino acid sequence AAS, or a variant thereof; a CDR-L3 comprising the amino acid sequence set forth in SEQ ID NO: 201, or a variant thereof; (10) the heavy chain immunoglobulin or variable region thereof comprises a CDR-H1 comprising the amino acid sequence set forth in SEQ ID NO: 209, or a variant thereof; a CDR-H2 comprising the amino acid sequence set forth in SEQ ID NO: 211, or a variant thereof; a CDR-H3 comprising the amino acid sequence set forth in SEQ ID NO: 213, or a variant thereof; and the light chain immunoglobulin or variable region thereof comprises a CDR-L1 comprising the amino acid sequence set forth in SEQ ID NO: 217, or a variant thereof; a CDR-L2 comprising the amino acid sequence KIS, or a variant thereof; a CDR-L3 comprising the amino acid sequence set forth in SEQ ID NO: 221, or a variant thereof; (11) the heavy chain immunoglobulin or variable region thereof comprises a CDR-H1 comprising the amino acid sequence set forth in SEQ ID NO: 229, or a variant thereof; a CDR-H2 comprising the amino acid sequence set forth in SEQ ID NO: 231, or a variant thereof; a CDR-H3 comprising the amino acid sequence set forth in SEQ ID NO: 233, or a variant thereof; and the light chain immunoglobulin or variable region thereof comprises a CDR-L1 comprising the amino acid sequence set forth in SEQ ID NO: 237, or a variant thereof; a CDR-L2 comprising the amino acid sequence AAS, or a variant thereof; a CDR-L3 comprising the amino acid sequence set forth in SEQ ID NO: 241, or a variant thereof; (12) the heavy chain immunoglobulin or variable region thereof comprises a CDR-H1 comprising the amino acid sequence set forth in SEQ ID NO: 249, or a variant thereof; a CDR-H2 comprising the amino acid sequence set forth in SEQ ID NO: 251, or a variant thereof; a CDR-H3 comprising the amino acid sequence set forth in SEQ ID NO: 253, or a variant thereof; and the light chain immunoglobulin or variable region thereof comprises a CDR-L1 comprising the amino acid sequence set forth in SEQ ID NO: 257, or a variant thereof; a CDR-L2 comprising the amino acid sequence AAS, or a variant thereof; a CDR-L3 comprising the amino acid sequence set forth in SEQ ID NO: 261, or a variant thereof; (13) the heavy chain immunoglobulin or variable region thereof comprises a CDR-H1 comprising the amino acid sequence set forth in SEQ ID NO: 277, or a variant thereof; a CDR-H2 comprising the amino acid sequence set forth in SEQ ID NO: 279, or a variant thereof; a CDR-H3 comprising the amino acid sequence set forth in SEQ ID NO: 281, or a variant thereof; and the light chain immunoglobulin or variable region thereof comprises a CDR-L1 comprising the amino acid sequence set forth in SEQ ID NO: 285, or a variant thereof; a CDR-L2 comprising the amino acid sequence AAS, or a variant thereof; a CDR-L3 comprising the amino acid sequence set forth in SEQ ID NO: 289, or a variant thereof; (14) the heavy chain immunoglobulin or variable region thereof comprises a CDR-H1 comprising the amino acid sequence set forth in SEQ ID NO: 297, or a variant thereof; a CDR-H2 comprising the amino acid sequence set forth in SEQ ID NO: 299, or a variant thereof; a CDR-H3 comprising the amino acid sequence set forth in SEQ ID NO: 301, or a variant thereof; and the light chain immunoglobulin or variable region thereof comprises a CDR-L1 comprising the amino acid sequence set forth in SEQ ID NO: 305, or a variant thereof; a CDR-L2 comprising the amino acid sequence AAS, or a variant thereof; a CDR-L3 comprising the amino acid sequence set forth in SEQ ID NO: 309, or a variant thereof; (15) the heavy chain immunoglobulin or variable region thereof comprises a CDR-H1 comprising the amino acid sequence set forth in SEQ ID NO: 317, or a variant thereof; a CDR-H2 comprising the amino acid sequence set forth in SEQ ID NO: 319, or a variant thereof; a CDR-H3 comprising the amino acid sequence set forth in SEQ ID NO: 321, or a variant thereof; and the light chain immunoglobulin or variable region thereof comprises a CDR-L1 comprising the amino acid sequence set forth in SEQ ID NO: 325, or a variant thereof; a CDR-L2 comprising the amino acid sequence AAS, or a variant thereof; a CDR-L3 comprising the amino acid sequence set forth in SEQ ID NO: 329, or a variant thereof; (16) the heavy chain immunoglobulin or variable region thereof comprises a CDR-H1 comprising the amino acid sequence set forth in SEQ ID NO: 337, or a variant thereof; a CDR-H2 comprising the amino acid sequence set forth in SEQ ID NO: 339, or a variant thereof; a CDR-H3 comprising the amino acid sequence set forth in SEQ ID NO: 341, or a variant thereof; and the light chain immunoglobulin or variable region thereof comprises a CDR-L1 comprising the amino acid sequence set forth in SEQ ID NO: 345, or a variant thereof; a CDR-L2 comprising the amino acid sequence GAS, or a variant thereof; a CDR-L3 comprising the amino acid sequence set forth in SEQ ID NO: 349, or a variant thereof; (17) the heavy chain immunoglobulin or variable region thereof comprises a CDR-H1 comprising the amino acid sequence set forth in SEQ ID NO: 357, or a variant thereof; a CDR-H2 comprising the amino acid sequence set forth in SEQ ID NO: 359, or a variant thereof; a CDR-H3 comprising the amino acid sequence set forth in SEQ ID NO: 361, or a variant thereof; and the light chain immunoglobulin or variable region thereof comprises a CDR-L1 comprising the amino acid sequence set forth in SEQ ID NO: 365, or a variant thereof; a CDR-L2 comprising the amino acid sequence GAS, or a variant thereof; a CDR-L3 comprising the amino acid sequence set forth in SEQ ID NO: 369, or a variant thereof; (18) the heavy chain immunoglobulin or variable region thereof comprises a CDR-H1 comprising the amino acid sequence set forth in SEQ ID NO: 377, or a variant thereof; a CDR-H2 comprising the amino acid sequence set forth in SEQ ID NO: 379, or a variant thereof; a CDR-H3 comprising the amino acid sequence set forth in SEQ ID NO: 381, or a variant thereof; and the light chain immunoglobulin or variable region thereof comprises a CDR-L1 comprising the amino acid sequence set forth in SEQ ID NO: 385, or a variant thereof; a CDR-L2 comprising the amino acid sequence GAS, or a variant thereof; a CDR-L3 comprising the amino acid sequence set forth in SEQ ID NO: 389, or a variant thereof; or (19) the heavy chain immunoglobulin or variable region thereof comprises a CDR-H1 comprising the amino acid sequence set forth in SEQ ID NO: 397, or a variant thereof; a CDR-H2 comprising the amino acid sequence set forth in SEQ ID NO: 399, or a variant thereof; a CDR-H3 comprising the amino acid sequence set forth in SEQ ID NO: 401, or a variant thereof; and the light chain immunoglobulin or variable region thereof comprises a CDR-L1 comprising the amino acid sequence set forth in SEQ ID NO: 405, or a variant thereof; a CDR-L2 comprising the amino acid sequence GAS, or a variant thereof; a CDR-L3 comprising the amino acid sequence set forth in SEQ ID NO: 409, or a variant thereof. In an embodiment of the invention, the antibody or antigen-binding fragment thereof that binds specifically to GLP1R (e.g., BA of an ATDC as set forth herein) comprises: (a) the heavy chain immunoglobulin or variable region thereof comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411, or a variant thereof; and/or (b) the light chain immunoglobulin or variable region thereof comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413, or a variant thereof. In some embodiments, the heavy chain immunoglobulin does not comprise a C-terminal lysine or lysine and glycine.

In an embodiment of the invention, the antibody or antigen-binding fragment thereof that binds specifically to GLP1R (e.g., BA of an ATDC as set forth herein) comprises: (a) the heavy chain immunoglobulin variable region comprises the amino acid sequence set forth in SEQ ID NO: 26, and the light chain immunoglobulin variable region comprises the amino acid sequence set forth in SEQ ID NO: 34; (b) the heavy chain immunoglobulin variable region comprises the amino acid sequence set forth in SEQ ID NO: 46, and the light chain immunoglobulin variable region comprises the amino acid sequence set forth in SEQ ID NO: 54; (c) the heavy chain immunoglobulin variable region comprises the amino acid sequence set forth in SEQ ID NO: 66, and the light chain immunoglobulin variable region comprises the amino acid sequence set forth in SEQ ID NO: 74; (d) the heavy chain immunoglobulin variable region comprises the amino acid sequence set forth in SEQ ID NO: 86, and the light chain immunoglobulin variable region comprises the amino acid sequence set forth in SEQ ID NO: 94; (e) the heavy chain immunoglobulin variable region comprises the amino acid sequence set forth in SEQ ID NO: 106, and the light chain immunoglobulin variable region comprises the amino acid sequence set forth in SEQ ID NO: 114; (f) the heavy chain immunoglobulin variable region comprises the amino acid sequence set forth in SEQ ID NO: 126, and the light chain immunoglobulin variable region comprises the amino acid sequence set forth in SEQ ID NO: 134; (g) the heavy chain immunoglobulin variable region comprises the amino acid sequence set forth in SEQ ID NO: 146, and the light chain immunoglobulin variable region comprises the amino acid sequence set forth in SEQ ID NO: 154; (h) the heavy chain immunoglobulin variable region comprises the amino acid sequence set forth in SEQ ID NO: 166, and the light chain immunoglobulin variable region comprises the amino acid sequence set forth in SEQ ID NO: 174; (i) the heavy chain immunoglobulin variable region comprises the amino acid sequence set forth in SEQ ID NO: 187, and the light chain immunoglobulin variable region comprises the amino acid sequence set forth in SEQ ID NO: 195; (j) the heavy chain immunoglobulin variable region comprises the amino acid sequence set forth in SEQ ID NO: 207, and the light chain immunoglobulin variable region comprises the amino acid sequence set forth in SEQ ID NO: 215; (k) the heavy chain immunoglobulin variable region comprises the amino acid sequence set forth in SEQ ID NO: 227, and the light chain immunoglobulin variable region comprises the amino acid sequence set forth in SEQ ID NO: 235; (l) the heavy chain immunoglobulin variable region comprises the amino acid sequence set forth in SEQ ID NO: 247, and the light chain immunoglobulin variable region comprises the amino acid sequence set forth in SEQ ID NO: 255; (m) the heavy chain immunoglobulin variable region comprises the amino acid sequence set forth in SEQ ID NO: 275, and the light chain immunoglobulin variable region comprises the amino acid sequence set forth in SEQ ID NO: 283; (n) the heavy chain immunoglobulin variable region comprises the amino acid sequence set forth in SEQ ID NO: 295, and the light chain immunoglobulin variable region comprises the amino acid sequence set forth in SEQ ID NO: 303; (o) the heavy chain immunoglobulin variable region comprises the amino acid sequence set forth in SEQ ID NO: 315, and the light chain immunoglobulin variable region comprises the amino acid sequence set forth in SEQ ID NO: 323; (p) the heavy chain immunoglobulin variable region comprises the amino acid sequence set forth in SEQ ID NO: 335, and the light chain immunoglobulin variable region comprises the amino acid sequence set forth in SEQ ID NO: 343; (q) the heavy chain immunoglobulin variable region comprises the amino acid sequence set forth in SEQ ID NO: 355, and the light chain immunoglobulin variable region comprises the amino acid sequence set forth in SEQ ID NO: 363; (r) the heavy chain immunoglobulin variable region comprises the amino acid sequence set forth in SEQ ID NO: 375, and the light chain immunoglobulin variable region comprises the amino acid sequence set forth in SEQ ID NO: 383; and/or (s) the heavy chain immunoglobulin variable region comprises the amino acid sequence set forth in SEQ ID NO: 395, and the light chain immunoglobulin variable region comprises the amino acid sequence set forth in SEQ ID NO: 403. In an embodiment of the invention, the antibody or antigen-binding fragment thereof that binds specifically to GLP1R (e.g., BA of an ATDC as set forth herein) comprises: (a) the heavy chain immunoglobulin comprises the amino acid sequence set forth in SEQ ID NO: 42, and the light chain immunoglobulin comprises the amino acid sequence set forth in SEQ ID NO: 44; (b) the heavy chain immunoglobulin comprises the amino acid sequence set forth in SEQ ID NO: 62, and the light chain immunoglobulin comprises the amino acid sequence set forth in SEQ ID NO: 64; (c) the heavy chain immunoglobulin comprises the amino acid sequence set forth in SEQ ID NO: 82, and the light chain immunoglobulin comprises the amino acid sequence set forth in SEQ ID NO: 84; (d) the heavy chain immunoglobulin comprises the amino acid sequence set forth in SEQ ID NO: 102, and the light chain immunoglobulin comprises the amino acid sequence set forth in SEQ ID NO: 104; (e) the heavy chain immunoglobulin comprises the amino acid sequence set forth in SEQ ID NO: 122, and the light chain immunoglobulin comprises the amino acid sequence set forth in SEQ ID NO: 124; (f) the heavy chain immunoglobulin comprises the amino acid sequence set forth in SEQ ID NO: 142, and the light chain immunoglobulin comprises the amino acid sequence set forth in SEQ ID NO: 144; (g) the heavy chain immunoglobulin comprises the amino acid sequence set forth in SEQ ID NO: 162, and the light chain immunoglobulin comprises the amino acid sequence set forth in SEQ ID NO: 164; (h) the heavy chain immunoglobulin comprises the amino acid sequence set forth in SEQ ID NO: 182, and the light chain immunoglobulin comprises the amino acid sequence set forth in SEQ ID NO: 184; (i) the heavy chain immunoglobulin comprises the amino acid sequence set forth in SEQ ID NO: 203, and the light chain immunoglobulin comprises the amino acid sequence set forth in SEQ ID NO: 205; (j) the heavy chain immunoglobulin comprises the amino acid sequence set forth in SEQ ID NO: 223, and the light chain immunoglobulin comprises the amino acid sequence set forth in SEQ ID NO: 225; (k) the heavy chain immunoglobulin comprises the amino acid sequence set forth in SEQ ID NO: 243, and the light chain immunoglobulin comprises the amino acid sequence set forth in SEQ ID NO: 245; (l) the heavy chain immunoglobulin comprises the amino acid sequence set forth in SEQ ID NO: 263, and the light chain immunoglobulin comprises the amino acid sequence set forth in SEQ ID NO: 265; (m) the heavy chain immunoglobulin comprises the amino acid sequence set forth in SEQ ID NO: 267, and the light chain immunoglobulin comprises the amino acid sequence set forth in SEQ ID NO: 269; (n) the heavy chain immunoglobulin comprises the amino acid sequence set forth in SEQ ID NO: 271, and the light chain immunoglobulin comprises the amino acid sequence set forth in SEQ ID NO: 273; (o) the heavy chain immunoglobulin comprises the amino acid sequence set forth in SEQ ID NO: 291, and the light chain immunoglobulin comprises the amino acid sequence set forth in SEQ ID NO: 293; (p) the heavy chain immunoglobulin comprises the amino acid sequence set forth in SEQ ID NO: 311, and the light chain immunoglobulin comprises the amino acid sequence set forth in SEQ ID NO: 313; (q) the heavy chain immunoglobulin comprises the amino acid sequence set forth in SEQ ID NO: 331, and the light chain immunoglobulin comprises the amino acid sequence set forth in SEQ ID NO: 333; (r) the heavy chain immunoglobulin comprises the amino acid sequence set forth in SEQ ID NO: 351, and the light chain immunoglobulin comprises the amino acid sequence set forth in SEQ ID NO: 353; (s) the heavy chain immunoglobulin comprises the amino acid sequence set forth in SEQ ID NO: 371, and the light chain immunoglobulin comprises the amino acid sequence set forth in SEQ ID NO: 373; (t) the heavy chain immunoglobulin comprises the amino acid sequence set forth in SEQ ID NO: 391, and the light chain immunoglobulin comprises the amino acid sequence set forth in SEQ ID NO: 393; or (u) the heavy chain immunoglobulin comprises the amino acid sequence set forth in SEQ ID NO: 411, and the light chain immunoglobulin comprises the amino acid sequence set forth in SEQ ID NO: 413. The present invention provides an antibody-tethered drug conjugate comprising a Glucagon-like peptide-1 receptor (GLP1R)-targeting antibody or an antigen-binding fragment thereof comprising: (a) the heavy chain immunoglobulin comprises the amino acid sequence set forth in SEQ ID NO: 42, and the light chain immunoglobulin comprises the amino acid sequence set forth in SEQ ID NO: 44; (b) the heavy chain immunoglobulin comprises the amino acid sequence set forth in SEQ ID NO: 62, and the light chain immunoglobulin comprises the amino acid sequence set forth in SEQ ID NO: 64; (c) the heavy chain immunoglobulin comprises the amino acid sequence set forth in SEQ ID NO: 82, and the light chain immunoglobulin comprises the amino acid sequence set forth in SEQ ID NO: 84; (d) the heavy chain immunoglobulin comprises the amino acid sequence set forth in SEQ ID NO: 102, and the light chain immunoglobulin comprises the amino acid sequence set forth in SEQ ID NO: 104; (e) the heavy chain immunoglobulin comprises the amino acid sequence set forth in SEQ ID NO: 122, and the light chain immunoglobulin comprises the amino acid sequence set forth in SEQ ID NO: 124; (f) the heavy chain immunoglobulin comprises the amino acid sequence set forth in SEQ ID NO: 142, and the light chain immunoglobulin comprises the amino acid sequence set forth in SEQ ID NO: 144; (g) the heavy chain immunoglobulin comprises the amino acid sequence set forth in SEQ ID NO: 162, and the light chain immunoglobulin comprises the amino acid sequence set forth in SEQ ID NO: 164; (h) the heavy chain immunoglobulin comprises the amino acid sequence set forth in SEQ ID NO: 182, and the light chain immunoglobulin comprises the amino acid sequence set forth in SEQ ID NO: 184; (i) the heavy chain immunoglobulin comprises the amino acid sequence set forth in SEQ ID NO: 203, and the light chain immunoglobulin comprises the amino acid sequence set forth in SEQ ID NO: 205; (j) the heavy chain immunoglobulin comprises the amino acid sequence set forth in SEQ ID NO: 223, and the light chain immunoglobulin comprises the amino acid sequence set forth in SEQ ID NO: 225; (k) the heavy chain immunoglobulin comprises the amino acid sequence set forth in SEQ ID NO: 243, and the light chain immunoglobulin comprises the amino acid sequence set forth in SEQ ID NO: 245; (l) the heavy chain immunoglobulin comprises the amino acid sequence set forth in SEQ ID NO: 263, and the light chain immunoglobulin comprises the amino acid sequence set forth in SEQ ID NO: 265; (m) the heavy chain immunoglobulin comprises the amino acid sequence set forth in SEQ ID NO: 267, and the light chain immunoglobulin comprises the amino acid sequence set forth in SEQ ID NO: 269; (n) the heavy chain immunoglobulin comprises the amino acid sequence set forth in SEQ ID NO: 271, and the light chain immunoglobulin comprises the amino acid sequence set forth in SEQ ID NO: 273; (o) the heavy chain immunoglobulin comprises the amino acid sequence set forth in SEQ ID NO: 291, and the light chain immunoglobulin comprises the amino acid sequence set forth in SEQ ID NO: 293; (p) the heavy chain immunoglobulin comprises the amino acid sequence set forth in SEQ ID NO: 311, and the light chain immunoglobulin comprises the amino acid sequence set forth in SEQ ID NO: 313; (q) the heavy chain immunoglobulin comprises the amino acid sequence set forth in SEQ ID NO: 331, and the light chain immunoglobulin comprises the amino acid sequence set forth in SEQ ID NO: 333; (r) the heavy chain immunoglobulin comprises the amino acid sequence set forth in SEQ ID NO: 351, and the light chain immunoglobulin comprises the amino acid sequence set forth in SEQ ID NO: 353; (s) the heavy chain immunoglobulin comprises the amino acid sequence set forth in SEQ ID NO: 371, and the light chain immunoglobulin comprises the amino acid sequence set forth in SEQ ID NO: 373; (t) the heavy chain immunoglobulin comprises the amino acid sequence set forth in SEQ ID NO: 391, and the light chain immunoglobulin comprises the amino acid sequence set forth in SEQ ID NO: 393; (u) the heavy chain immunoglobulin comprises the amino acid sequence set forth in SEQ ID NO: 411, and the light chain immunoglobulin comprises the amino acid sequence set forth in SEQ ID NO: 413; (v) the heavy chain immunoglobulin comprises the amino acid sequence set forth in SEQ ID NO: 414, and the light chain immunoglobulin comprises the amino acid sequence set forth in SEQ ID NO: 84; or (w) the heavy chain immunoglobulin comprises the amino acid sequence set forth in SEQ ID NO: 416, and the light chain immunoglobulin comprises the amino acid sequence set forth in SEQ ID NO: 84; wherein the structure of a linker-payload that is conjugated to amino acid 3 (Gln) of SEQ ID NOs: 44, 64, 84, 104, 124, 144, 164, 184, 205; 225; 245; 265; 269; 273; 293; 313; 333; 353; 373; 393; or 413 is represented by (SEQ ID NOS 506-507, respectively, in order of appearance):

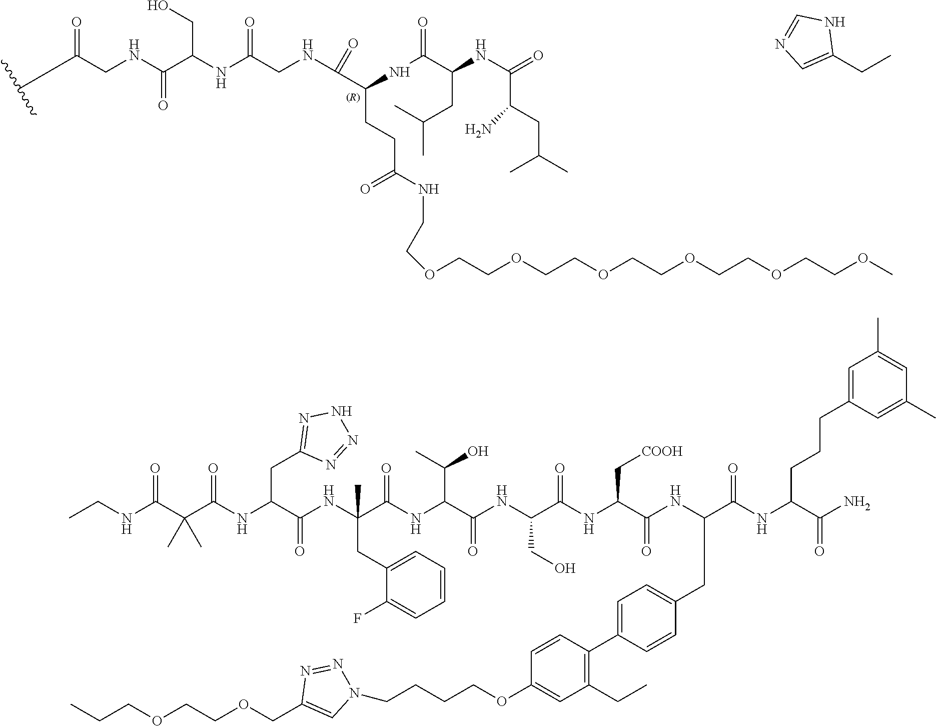

wherein

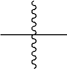

is the point of attachment of the amino acids 1-6 (LLQGSG (SEQ ID NO: 18) (included in structure above)) to amino acid 7 of SEQ ID NOs: 44, 64, 84, 104, 124, 144, 164, 184, 205; 225; 245; 265; 269; 273; 293; 313; 333; 353; 373; 393; or 413. In some embodiments, the heavy chain immunoglobulin does not comprise a C-terminal lysine or lysine and glycine.

The present invention provides an antibody-tethered drug conjugate comprising a Glucagon-like peptide-1 receptor (GLP1R)-targeting antibody or an antigen-binding fragment thereof comprising:

(a) the heavy chain immunoglobulin comprises the amino acid sequence set forth in SEQ ID NO: 42, and the light chain immunoglobulin comprises the amino acid sequence set forth in SEQ ID NO: 44; (b) the heavy chain immunoglobulin comprises the amino acid sequence set forth in SEQ ID NO: 62, and the light chain immunoglobulin comprises the amino acid sequence set forth in SEQ ID NO: 64; (c) the heavy chain immunoglobulin comprises the amino acid sequence set forth in SEQ ID NO: 82, and the light chain immunoglobulin comprises the amino acid sequence set forth in SEQ ID NO: 84; (d) the heavy chain immunoglobulin comprises the amino acid sequence set forth in SEQ ID NO: 102, and the light chain immunoglobulin comprises the amino acid sequence set forth in SEQ ID NO: 104; (e) the heavy chain immunoglobulin comprises the amino acid sequence set forth in SEQ ID NO: 122, and the light chain immunoglobulin comprises the amino acid sequence set forth in SEQ ID NO: 124; (f) the heavy chain immunoglobulin comprises the amino acid sequence set forth in SEQ ID NO: 142, and the light chain immunoglobulin comprises the amino acid sequence set forth in SEQ ID NO: 144; (g) the heavy chain immunoglobulin comprises the amino acid sequence set forth in SEQ ID NO: 162, and the light chain immunoglobulin comprises the amino acid sequence set forth in SEQ ID NO: 164; (h) the heavy chain immunoglobulin comprises the amino acid sequence set forth in SEQ ID NO: 182, and the light chain immunoglobulin comprises the amino acid sequence set forth in SEQ ID NO: 184; (i) the heavy chain immunoglobulin comprises the amino acid sequence set forth in SEQ ID NO: 203, and the light chain immunoglobulin comprises the amino acid sequence set forth in SEQ ID NO: 205; (j) the heavy chain immunoglobulin comprises the amino acid sequence set forth in SEQ ID NO: 223, and the light chain immunoglobulin comprises the amino acid sequence set forth in SEQ ID NO: 225; (k) the heavy chain immunoglobulin comprises the amino acid sequence set forth in SEQ ID NO: 243, and the light chain immunoglobulin comprises the amino acid sequence set forth in SEQ ID NO: 245; (l) the heavy chain immunoglobulin comprises the amino acid sequence set forth in SEQ ID NO: 263, and the light chain immunoglobulin comprises the amino acid sequence set forth in SEQ ID NO: 265; (m) the heavy chain immunoglobulin comprises the amino acid sequence set forth in SEQ ID NO: 267, and the light chain immunoglobulin comprises the amino acid sequence set forth in SEQ ID NO: 269; (n) the heavy chain immunoglobulin comprises the amino acid sequence set forth in SEQ ID NO: 271, and the light chain immunoglobulin comprises the amino acid sequence set forth in SEQ ID NO: 273; (o) the heavy chain immunoglobulin comprises the amino acid sequence set forth in SEQ ID NO: 291, and the light chain immunoglobulin comprises the amino acid sequence set forth in SEQ ID NO: 293; (p) the heavy chain immunoglobulin comprises the amino acid sequence set forth in SEQ ID NO: 311, and the light chain immunoglobulin comprises the amino acid sequence set forth in SEQ ID NO: 313; (q) the heavy chain immunoglobulin comprises the amino acid sequence set forth in SEQ ID NO: 331, and the light chain immunoglobulin comprises the amino acid sequence set forth in SEQ ID NO: 333; (r) the heavy chain immunoglobulin comprises the amino acid sequence set forth in SEQ ID NO: 351, and the light chain immunoglobulin comprises the amino acid sequence set forth in SEQ ID NO: 353; (s) the heavy chain immunoglobulin comprises the amino acid sequence set forth in SEQ ID NO: 371, and the light chain immunoglobulin comprises the amino acid sequence set forth in SEQ ID NO: 373; (t) the heavy chain immunoglobulin comprises the amino acid sequence set forth in SEQ ID NO: 391, and the light chain immunoglobulin comprises the amino acid sequence set forth in SEQ ID NO: 393; (u) the heavy chain immunoglobulin comprises the amino acid sequence set forth in SEQ ID NO: 411, and the light chain immunoglobulin comprises the amino acid sequence set forth in SEQ ID NO: 413; (v) the heavy chain immunoglobulin comprises the amino acid sequence set forth in SEQ ID NO: 414, and the light chain immunoglobulin comprises the amino acid sequence set forth in SEQ ID NO: 84; or (w) the heavy chain immunoglobulin comprises the amino acid sequence set forth in SEQ ID NO: 416, and the light chain immunoglobulin comprises the amino acid sequence set forth in SEQ ID NO: 84; wherein the structure of a linker-payload that is conjugated to amino acid 3 (Gln) of SEQ ID NOs: 44, 64, 84, 104, 124, 144, 164, 184, 205; 225; 245; 265; 269; 273; 293; 313; 333; 353; 373; 393; or 413 is represented by (SEQ ID NOS 506-507, respectively, in order of appearance):

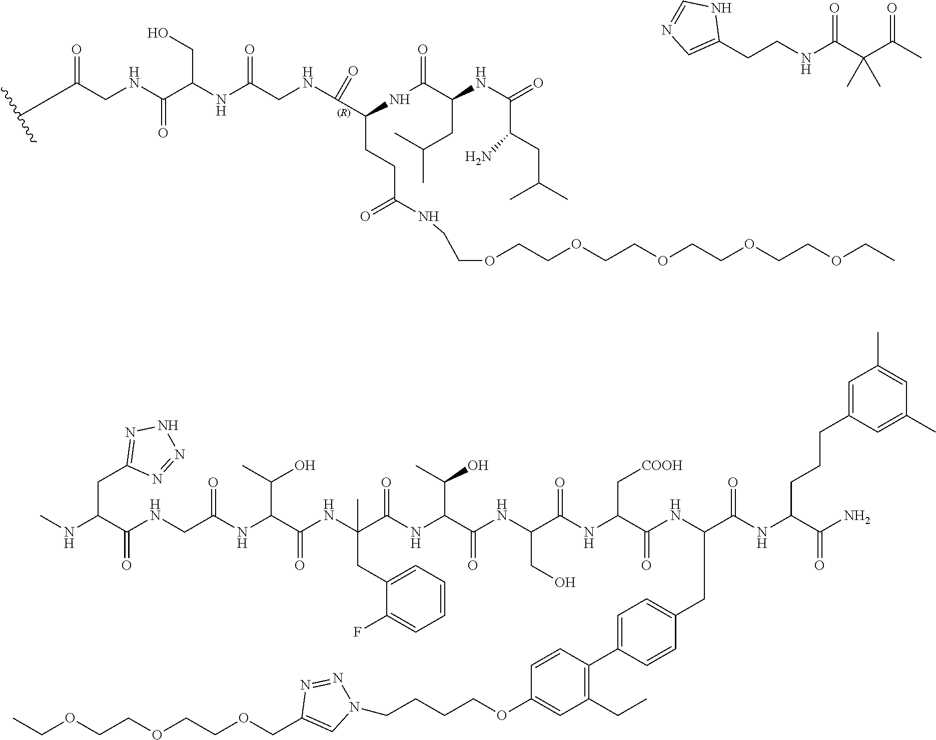

wherein

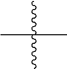

is the point of attachment of the amino acids 1-6 (LLQGSG (SEQ ID NO: 18) (included in structure above)) to amino acid 7 of SEQ ID NOs: 44, 64, 84, 104, 124, 144, 164, 184, 205; 225; 245; 265; 269; 273; 293; 313; 333; 353; 373; 393; or 413. In some embodiments, the heavy chain immunoglobulin does not comprise a C-terminal lysine or lysine and glycine.

The present invention also provides an antibody-tethered drug conjugate (ATDC) comprising an antibody or antigen-binding fragment thereof that binds specifically to GLP1R and a payload that is conjugated to a linker which is conjugated to one or both of two immunoglobulin heavy chains or variable regions thereof and/or one or both of two immunoglobulin light chains or variable regions thereof of the antibody or fragment which is characterized by the structure disclosed as SEQ ID NO: 447:

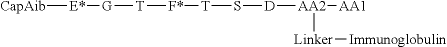

wherein
immunoglobulin is the immunoglobulin chain of the antibody or fragment (e.g., the light chain immunoglobulin);
Linker is a linker as discussed herein;
CapAib is 3-((2-(1H-imidazol-5-yl)ethyl)amino)-2,2-dimethyl-3-oxopropanoic acid;
E* is (S)-2-amino-3-(2H-tetrazol-5-yl)propanoic acid;
G is glycine;
T is threonine;
F* is (S)-2-amino-3-(2-fluorophenyl)-2-methylpropanoic acid;
S is serine;
D is aspartate;
AA2 is (S)-2-amino-3-(4′-(4-(4-(25-amino-2,5,8,11,14,17,20,23-octaoxapentacosyl)-1H-1,2,3-triazol-1-yl)butoxy)-2′-ethyl-[1,1′-biphenyl]-4-yl)propanoic acid [AA2 includes linker]; and
AA1=(S)-2-amino-5-(3,5-dimethylphenyl)pentanamide. The structure disclosed as SEQ ID NO: 447:

CapAib-E*-G-T-F*-T-S-D-AA2-AA1

includes, for example, G-T or CapAib-E*, which indicates that these residues are joined by a bond, e.g., a peptide bond. In an embodiment of the invention, the antibody or fragment includes a Qtag including the amino acid sequence LLQGSG (SEQ ID NO: 18) in both of the immunoglobulin light chains. A linker, in the linker-payload, having the structure R—NH₂ (e.g., including the general structure H₂N-linker-payload]) may be conjugated to the side-group of the Qtag glutamine (Gln) at the sidechain —C(═O)—NH₂ via a transglutaminase reaction, e.g., according to the following reaction diagram:

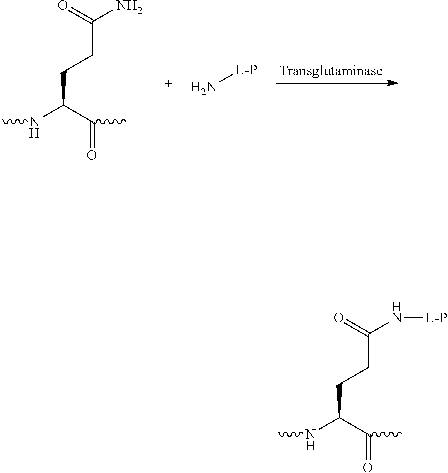

* H₂N-L-P is a linker-payload having an —NH₂ group.

This reaction may be referred to as aminylation. Aminylation refers to the process by which primary amines, e.g., of a linker-payload, are covalently coupled to a peptide-bound glutamine residue by a transglutaminase. When transglutaminase is in the vicinity of a peptide Gln residue, and there are primary amine substrates available (e.g., a linker-payload having a primary amine, such as M3190), the enzyme catalyzes the incorporation of the primary amino group to glutamine resulting in the formation of a gamma-glutamyl-amine bond. The result of such a reaction may be referred to herein as a “aminylation product”. An aminylation product may, but not necessarily, be the product of the catalysis of two molecules by a transglutaminase enzyme. For example, an aminylation product may be the result of chemical synthesis without use of a transglutaminase enzyme. See Lai et al., Tissue transglutaminase (TG2) and mitochondrial function and dysfunction, Frontiers in Bioscience-Landmark. 2017. 22(7); 1114-1137.

The present invention also provides a method of selectively targeting GLP1R on the surface of a cell (e.g., in the body of a subject or in vitro) for delivery of a payload (e.g., L11, L30 or L32) with an ATDC of any of the embodiments described herein (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) that comprises the steps of contacting contacting the cell with the ATDC. In an embodiment of the invention, the method comprises the step of administering the ATDC or a pharmaceutical composition thereof, to a subject in whose body the cell exists. In some embodiments, the cell is a mammalian cell. In some embodiments, the cell is a human cell. In some embodiments, the cell is a pancreatic cell, a brain cell, a heart cell, a vascular tissue cell, a kidney cell, an adipose tissue cell, a liver cell, or a muscle cell.

The present invention provides a method of enhancing GLP1R activity in a subject in need thereof comprising administering to the subject an effective amount of the ATDC of any of the embodiments described herein (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32), the composition described herein, or the dosage form described herein. In an embodiment of the invention, the subject suffers from a GLP1R-associated condition (e.g., obesity and/or diabetes (type 1 or type 2)).

In another aspect, provided herein is a method of lowering blood glucose levels in a subject in need thereof comprising administering to the subject an effective amount of ATDC of any of the embodiments described herein (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32), the composition described herein, or the dosage form described herein. In an embodiment of the invention, the subject suffers from a GLP1R-associated condition (e.g., obesity and/or diabetes (type 1 or type 2)).

In another aspect, provided herein is a method of lowering body weight in an individual in need thereof comprising administering to the individual an effective amount of ATDC of any of the embodiments described herein (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32), the composition described herein, or the dosage form described herein. In an embodiment of the invention, the subject suffers from a GLP1R-associated condition (e.g., obesity and/or diabetes (type 1 or type 2)).

In another aspect, provided herein is a method of treating a GLP1R-associated condition in a subject in need thereof comprising administering to the subject an effective amount of ATDC of any of the embodiments described herein (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32), the composition described herein, or the dosage form described herein. In some embodiments, the GLP1R-associated condition is type II diabetes, obesity, liver disease, coronary artery disease, or kidney disease. In some embodiments, the GLP1R-associated condition is type II diabetes and/or obesity.

In various embodiments of any of the method described herein, the ATDC, the composition, or the dosage form of the present disclosure (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) is administered subcutaneously, intravenously, intradermally, intraperitoneally, or intramuscularly.

In another aspect, provided herein is a method of producing the ATDC descrbed herein having a structure of Formula (A):

BA-(L-P)ₘ (A),

the method comprising the steps of:
a) contacting, in the presence of a transglutaminase, the BA comprising at least m glutamine residues Gln with at least m equivalents of compound L-P, and
b) isolating the produced ATDC of Formula (A);
   wherein BA is an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   optionally, wherein the heavy chain immunoglobulin does not comprise a C-terminal lysine or lysine and glycine.

In another aspect, provided herein is a method of producing the ATDC described herein having a structure of Formula (I):

BA-L-P (I),

the method comprising the steps of:
a) contacting, in the presence of a transglutaminase, the BA comprising at least one glutamine residue Gln with a compound L-P, and
b) isolating the produced ATDC of Formula (I);
   wherein, BA is an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   optionally, wherein the heavy chain immunoglobulin does not comprise a C-terminal lysine or lysine and glycine.

In another aspect, provided herein is a method of producing an ATDC having a structure of Formula (A):

BA-(L-P)ₘ (A),
wherein the linker L has has the structure of formula (L′):

—La—Y-Lp- (L′),

wherein La is a first linker covalently attached to the BA;
Y is a group comprising a triazole, and
Lp is a second linker covalently attached to the P,
   the method comprising the steps of:
a) contacting, in the presence of a transglutaminase, the BA comprising at least m glutamine residues Gln with the first linker La comprising an azide or an alkyne moiety;
b) contacting the product of step a) with at least m equivalents of compound Lp-P, wherein the second linker Lp comprises an azide or an alkyne moiety, wherein La and Lp are capable of reacting to produce a triazole, and
c) isolating the produced ATDC of Formula (A);
   wherein BA is an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   optionally, wherein the heavy chain immunoglobulin does not comprise a C-terminal lysine or lysine and glycine.

In another aspect, provided herein is a method of producing an ATDC described herein having a structure of Formula (I):

BA-L-P (I),
wherein the linker L has has the structure of formula (L′):

—La—Y-Lp- (L′),

wherein La is a first linker covalently attached to the BA;
Y is a group comprising a triazole, and
Lp is a second linker covalently attached to the P,
   the method comprising the steps of:
a) contacting, in the presence of a transglutaminase, the BA comprising at least one glutamine residue Gln with the first linker La comprising an azide or an alkyne moiety;
b) contacting the product of step a) with a compound Lp-P, wherein the second linker Lp comprises an azide or an alkyne moiety, wherein La and Lp are capable of reacting to produce a triazole, and
c) isolating the produced ATDC of Formula (I);
   wherein BA is an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   optionally, wherein the heavy chain immunoglobulin does not comprise a C-terminal lysine or lysine and glycine.

In another aspect, provided herein is an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i); which is conjugated, e.g., by a linker, to a compound having a structure selected from the group consisting of Formula (P-IB) (SEQ ID NO: 508), Formula (P-IIB) (SEQ ID NO: 509), and Formula (P-IIIB) (SEQ ID NO: 510):

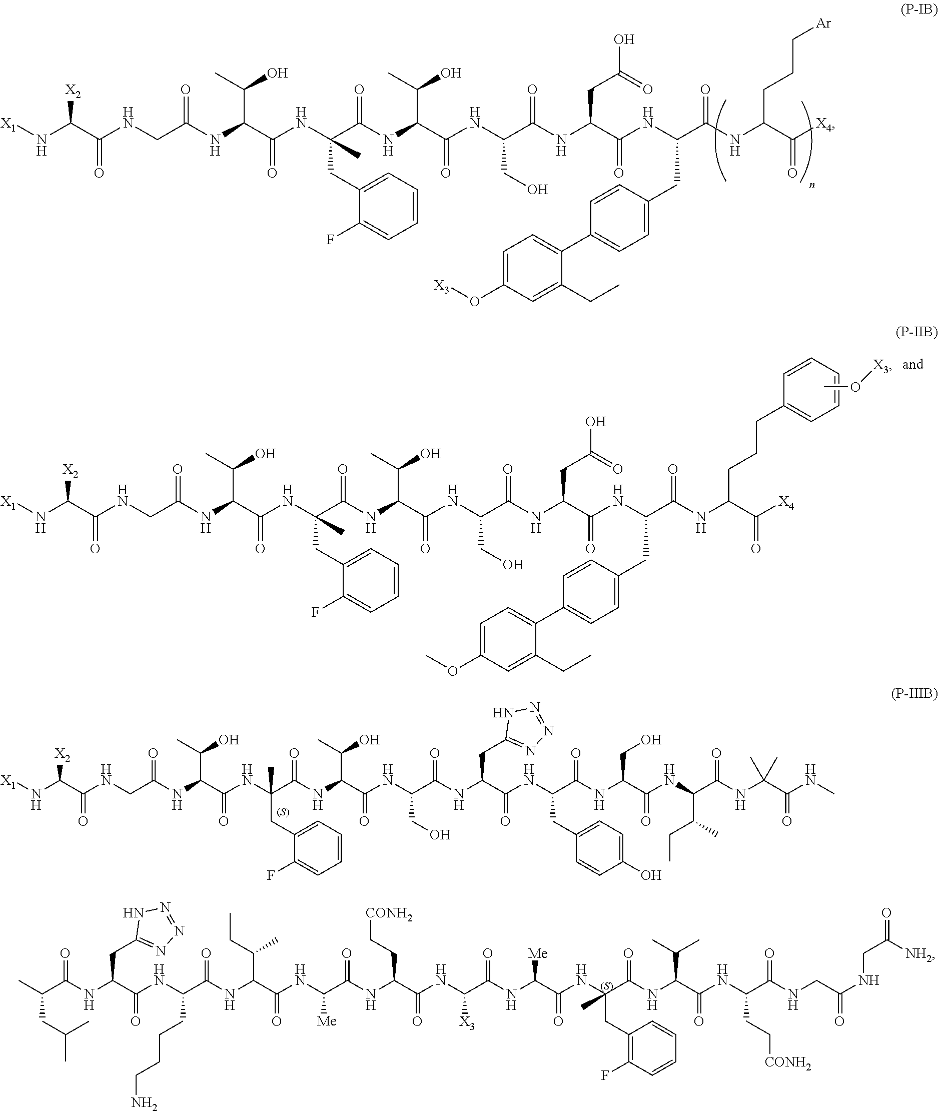

wherein:
X₁ is selected from H;

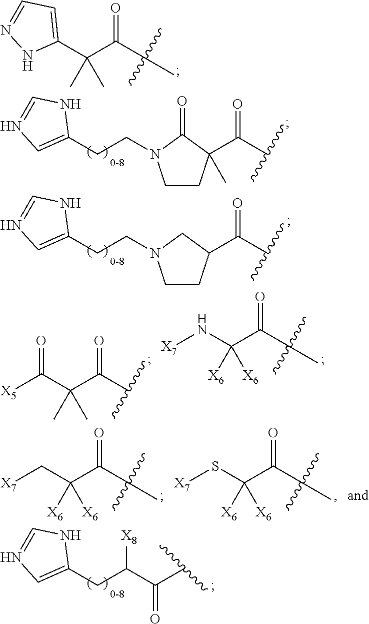

X₂ is selected from

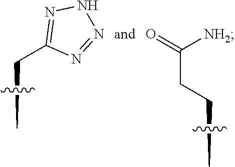

X₃ is selected from —CH₃, —(CH₂)₂₋₆—NH₂, —(CH₂)₂₋₆—N₃, and —(CH₂)₂₋₆-Tr-(CH₂)₁₋₆—NH₂, where Tr is a triazole moiety;
n is 0 or 1;
X₄ is selected from —NH₂, —OH and —N(H)(phenyl);
X₅ is selected from —OH, —NH₂, —NH—OH, and

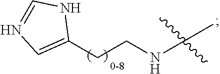

X₆ is independently at each occurrence selected from H, —OH, —CH₃, and —CH₂OH;
X₇ is selected from H,

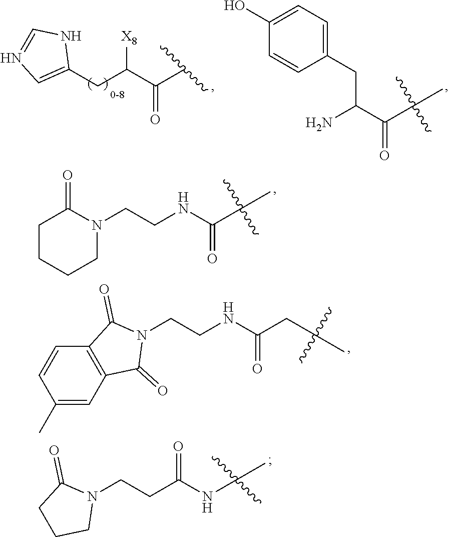

X₈ is selected from H, —OH, —NH₂, and

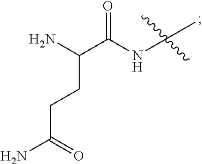 [SMILES: *NC(=O)C(N)CCC(N)=O]

Ar is selected from

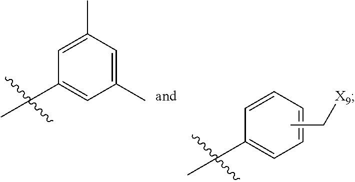

X₉ is selected from —NH₂,

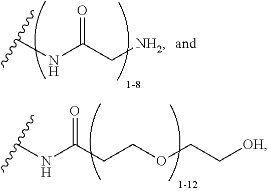

and
m is an integer from 1 to 4
or a pharmaceutically acceptable salt thereof;
optionally, wherein the heavy chain immunoglobulin does not comprise a C-terminal lysine or lysine and glycine.

In another aspect of the present invention, provided herein is an ATDC that includess an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   which is conjugated, e.g., by a linker, to a compound having a structure of Formula (II) (SEQ ID NO: 511):

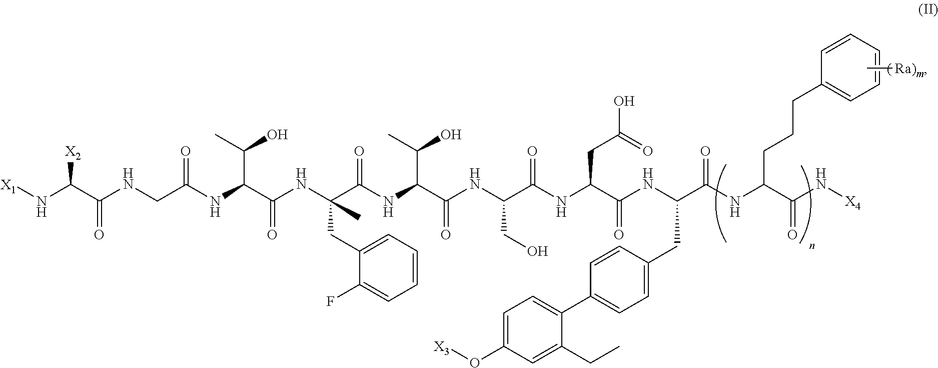

wherein:
X₁ is selected from H;

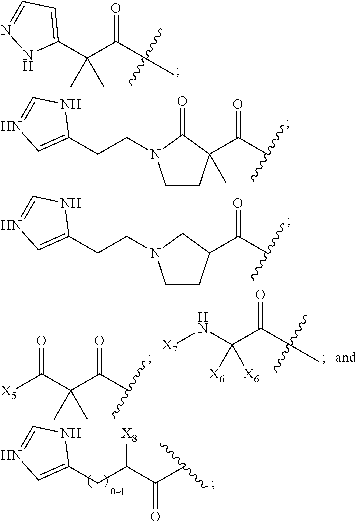

X₂ is selected from

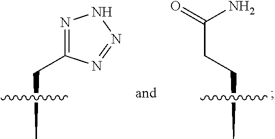

X₃ is selected from —(CH₂)₂₋₆—NH₂, —(CH₂)₂₋₆—N₃, and —CH₃, with the proviso that when X₃ is —CH₃, n is 1 and Ra in at least one occurrence is selected from —(CH₂)₂₋₆—NH₂ and —(CH₂)₂₋₆—N₃;
n is 0 or 1;
m is an integer from 0 to 3;
Ra is independently at each occurrence selected from —CH₃, —(CH₂)₂₋₆—NH₂, and —(CH₂)₂₋₆—N₃;
X₄ is selected from H and phenyl;
X₅ is selected from —OH, —NH₂, —NH—OH, and

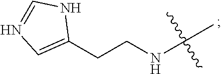 [SMILES: *NCCc1cnc[nH]1]

X₆ is independently at each occurrence selected from H, —OH, —CHs, and —CH₂OH;
X₇ is selected from H

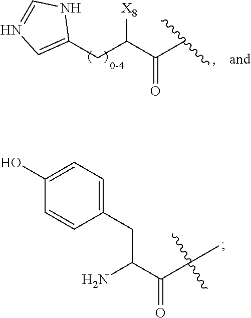

X₃ is selected from H, —OH, —NH₂, and

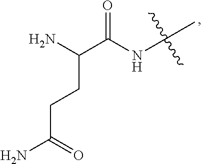 [SMILES: *NC(=O)C(N)CCC(N)=O]

and pharmaceutically acceptable salts thereof;
optionally, wherein the heavy chain immunoglobulin does not comprise a C-terminal lysine or lysine and glycine.

In some embodiments, P (Payload), in an ATDC that is set forth herein, has a structure selected from the group consisting of (SEQ ID NOS 465, 576, 466-495, 610, 496-497, 611, 498-505, respectively, in order of appearance):

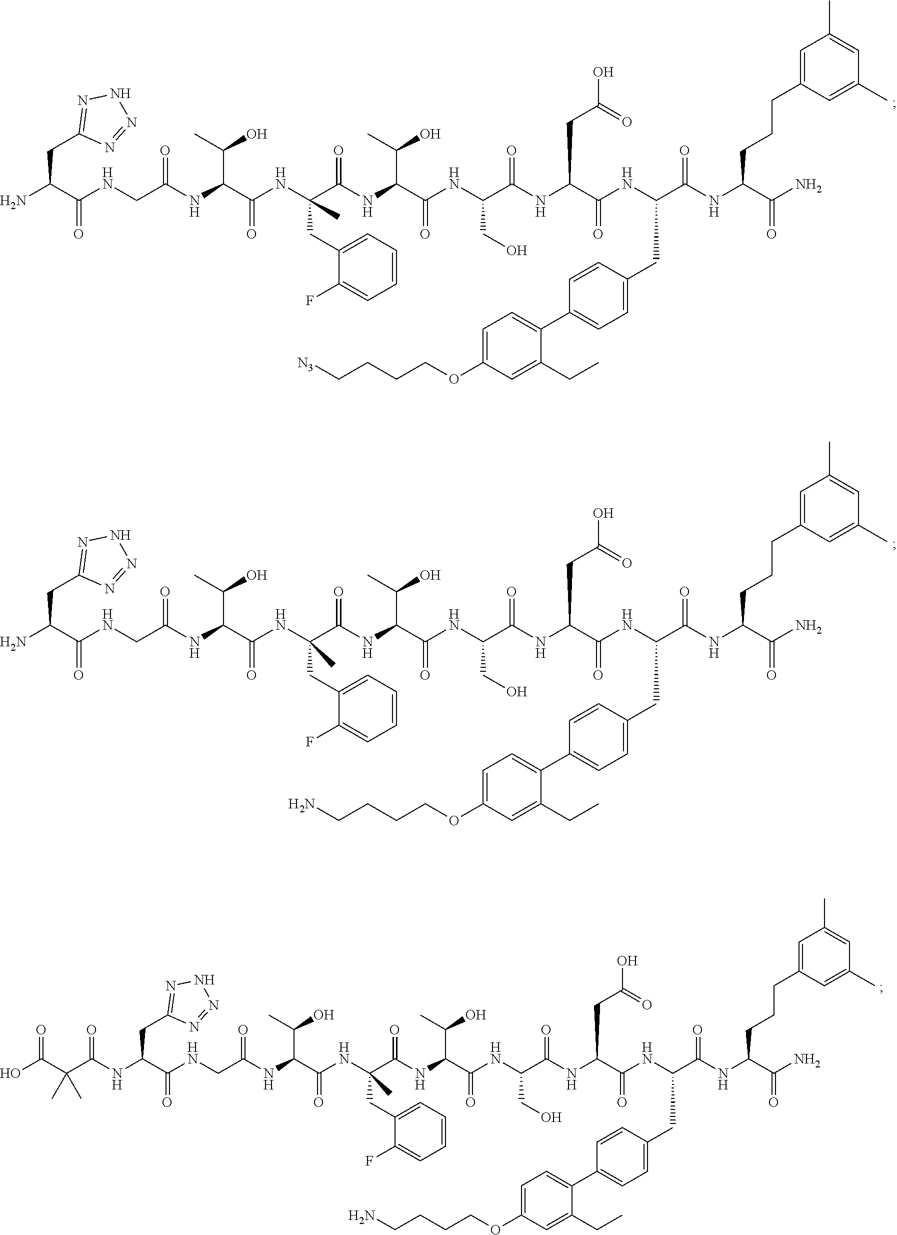

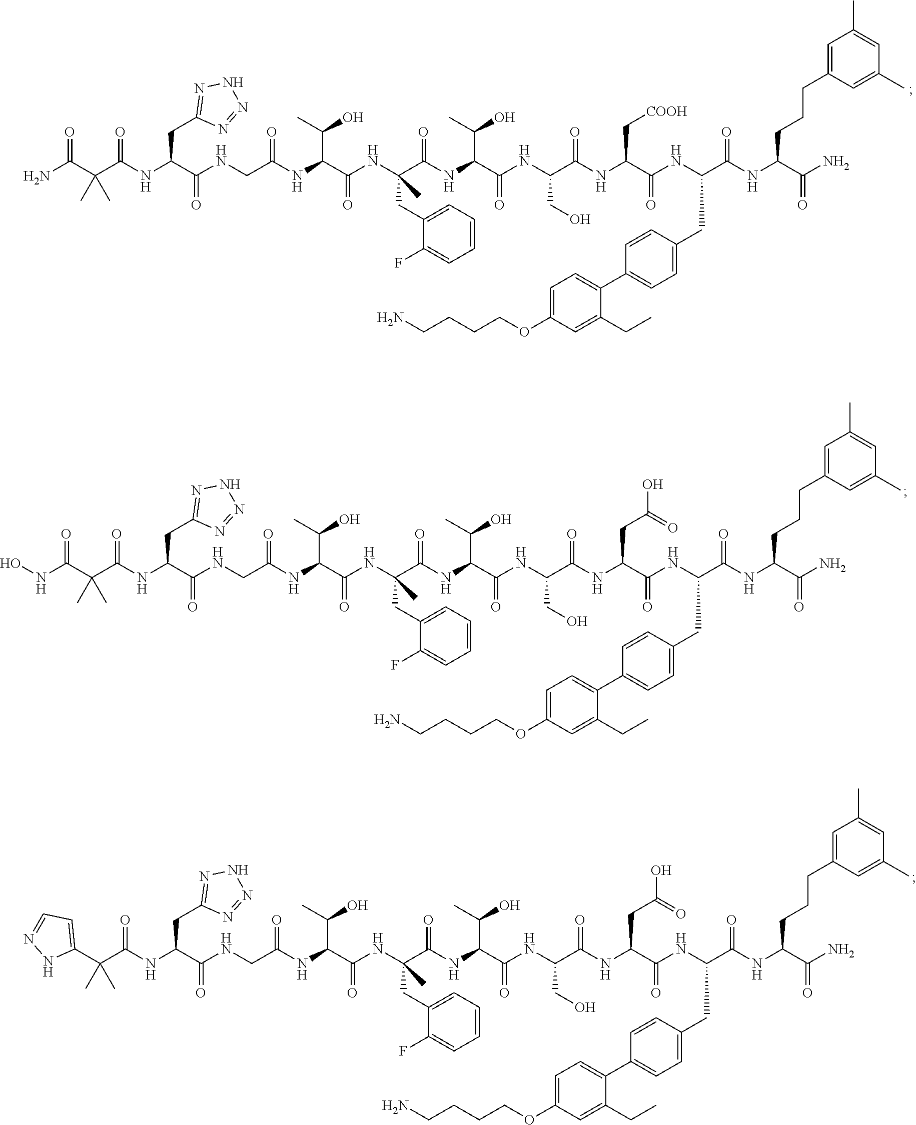

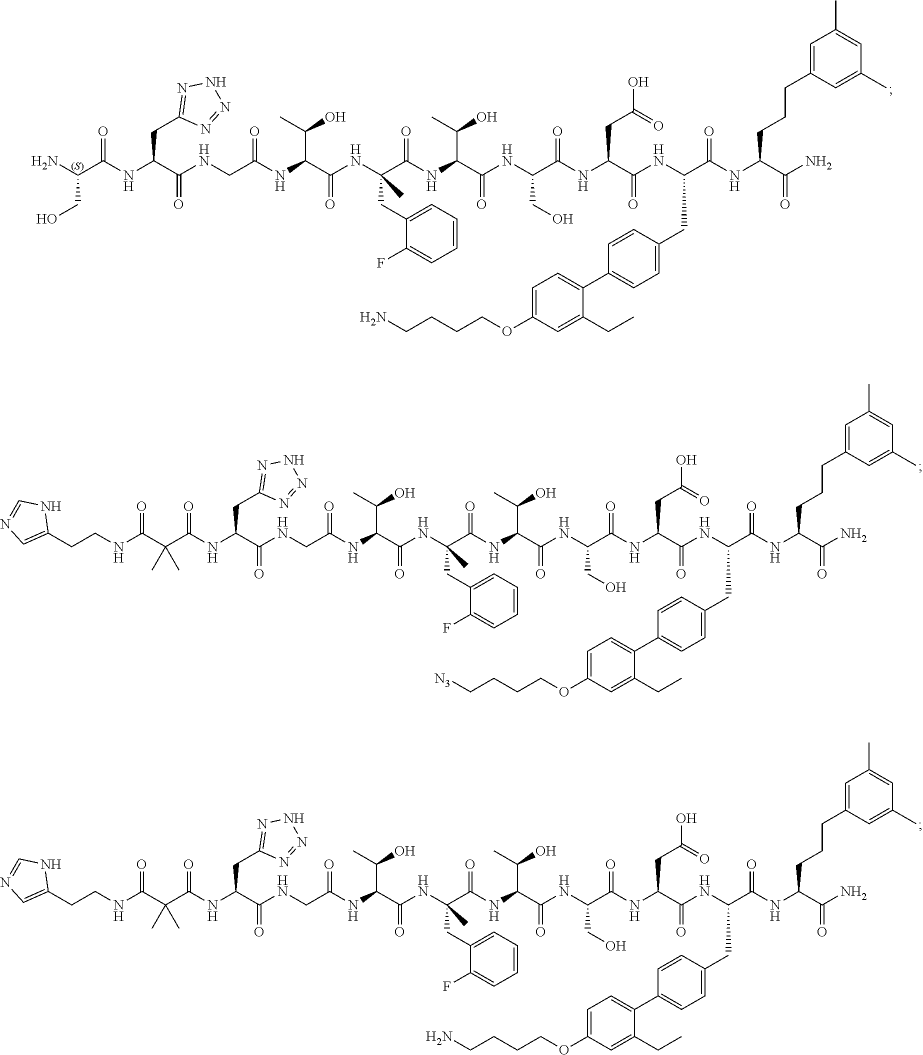

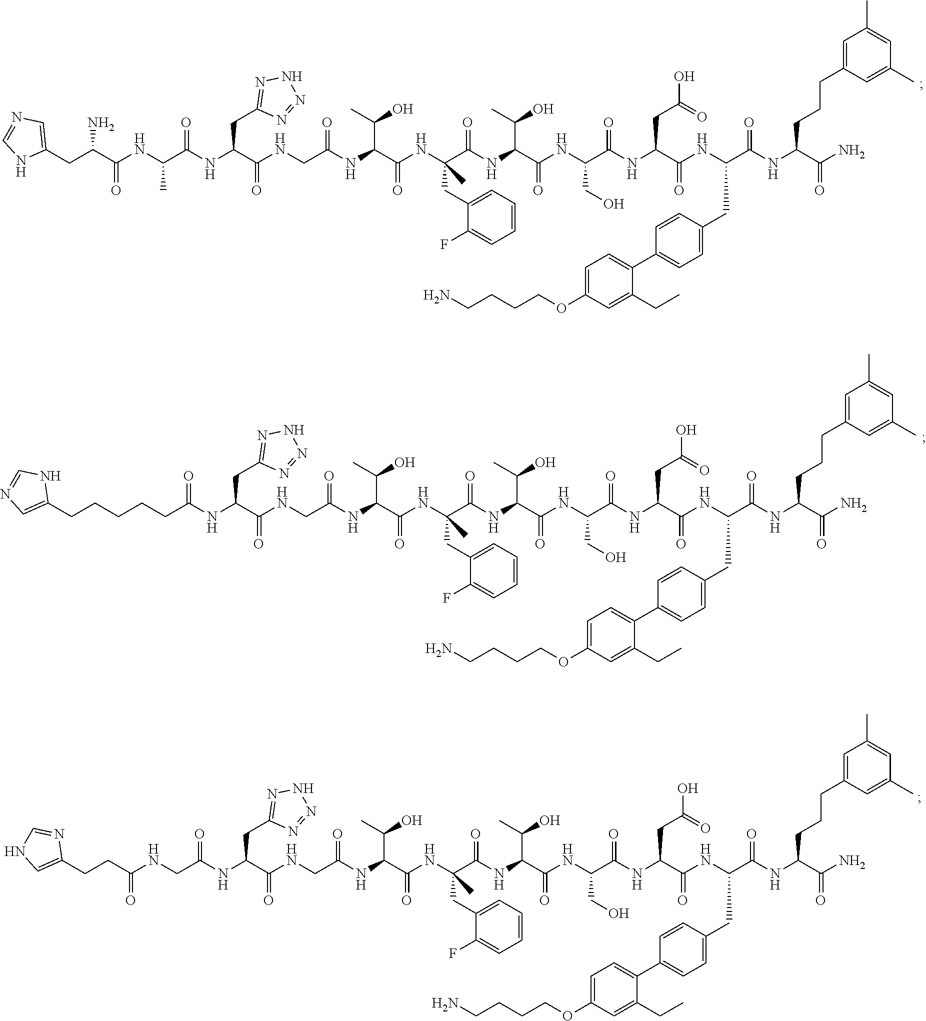

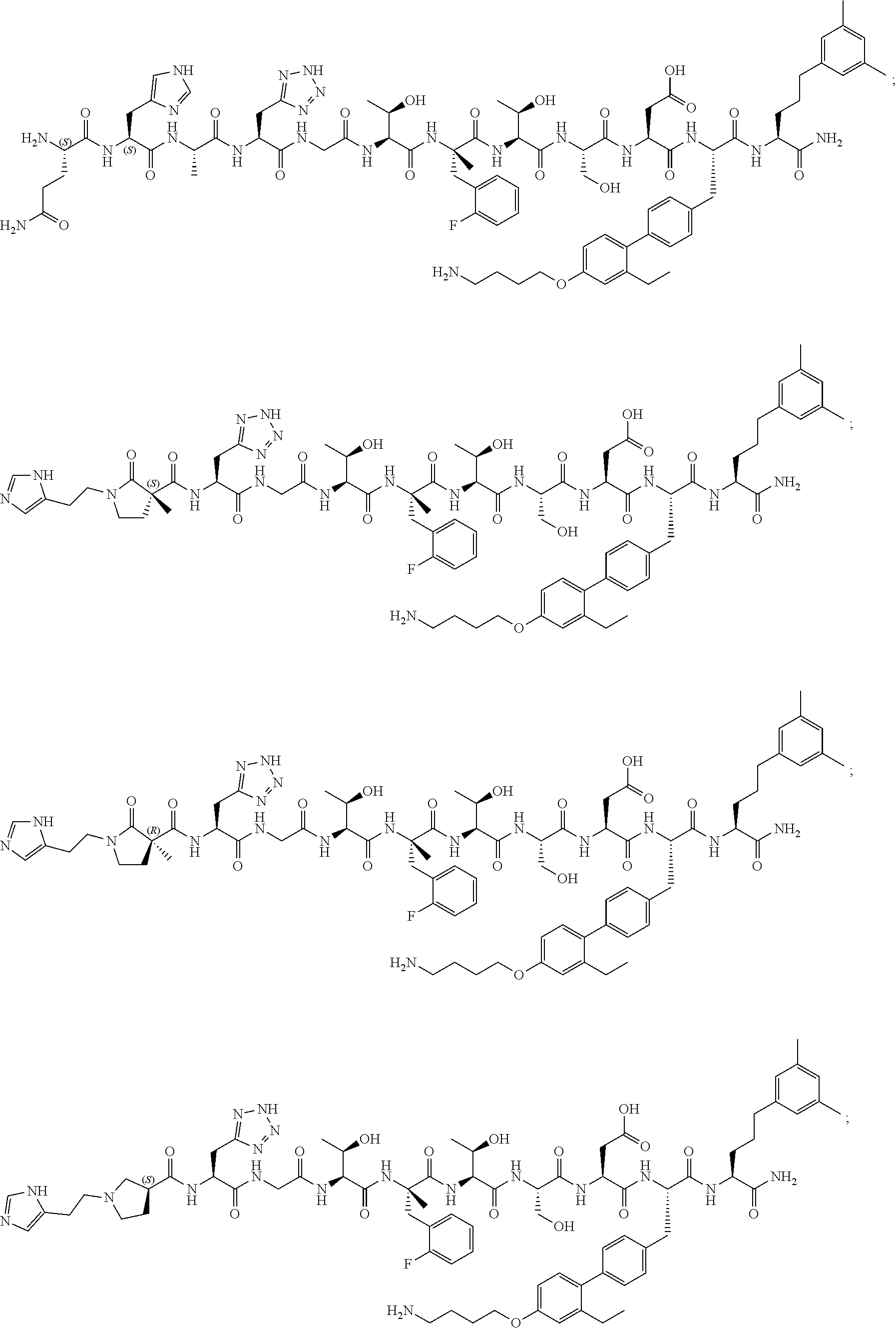

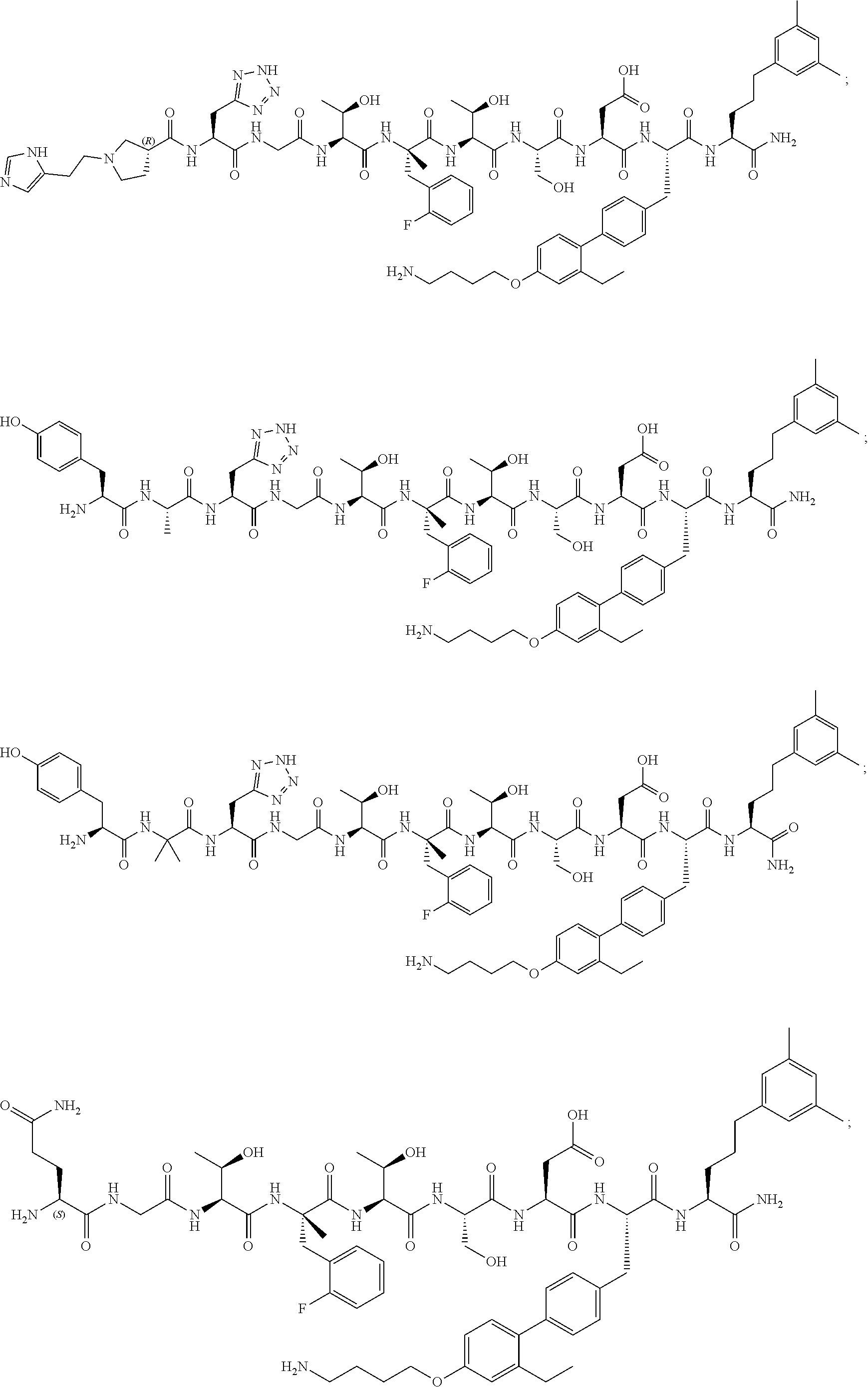

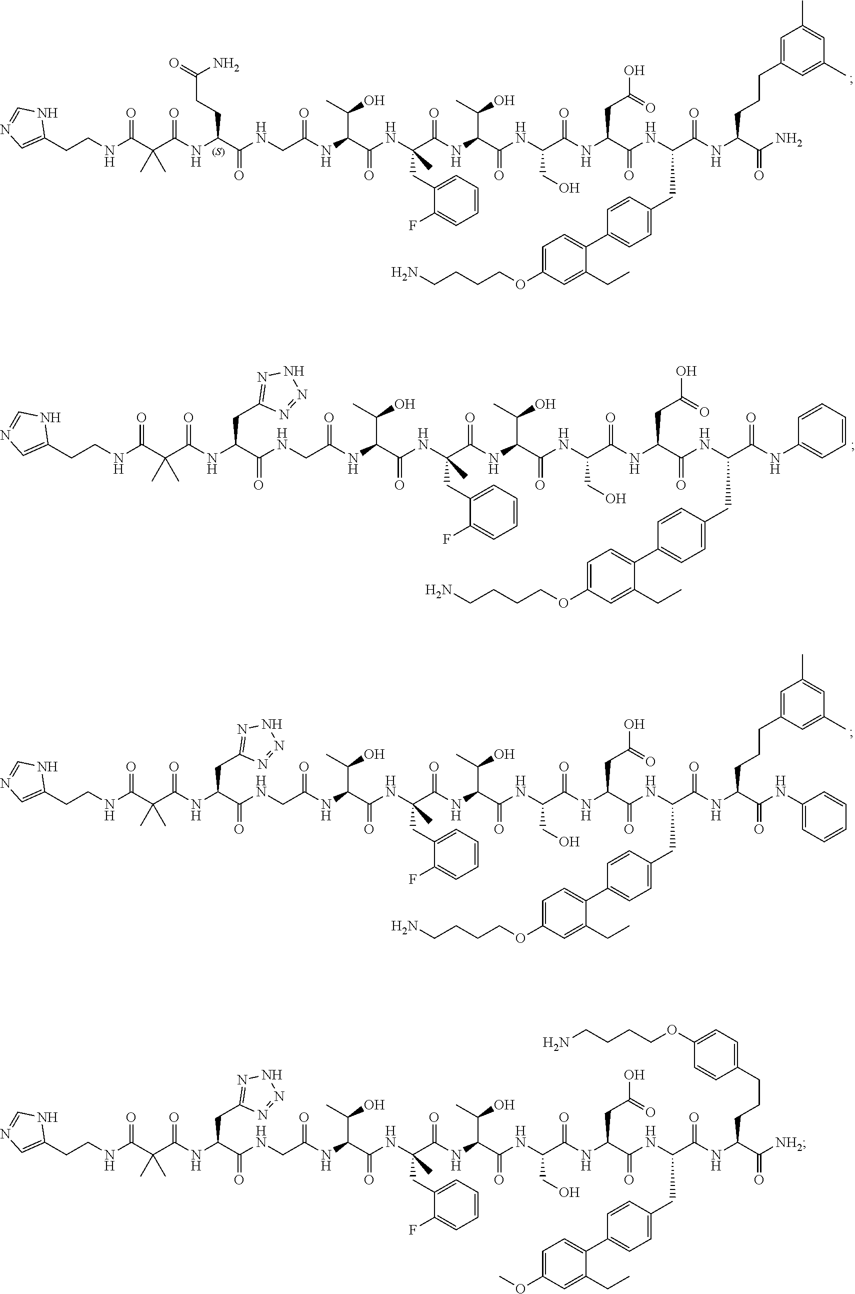

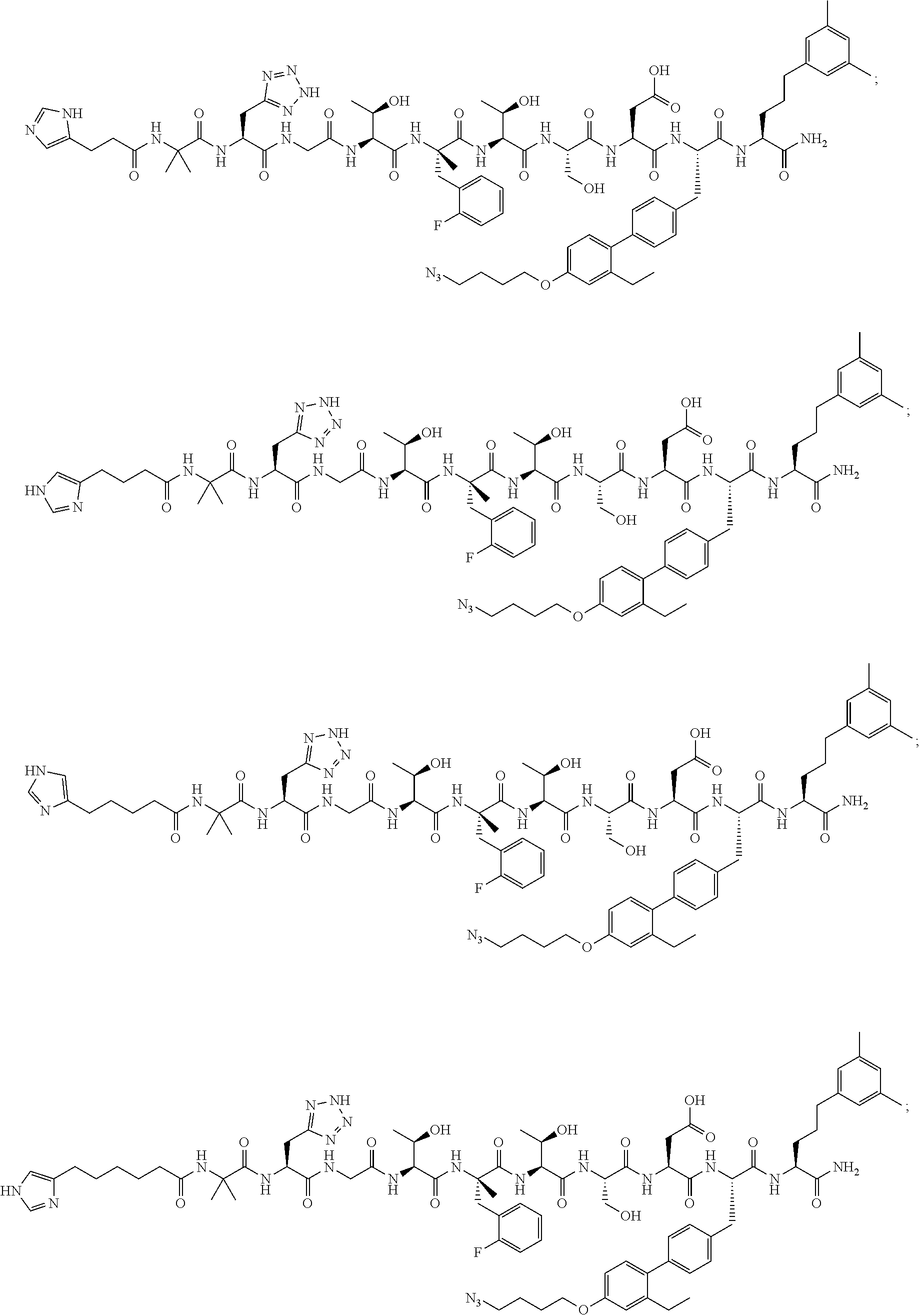

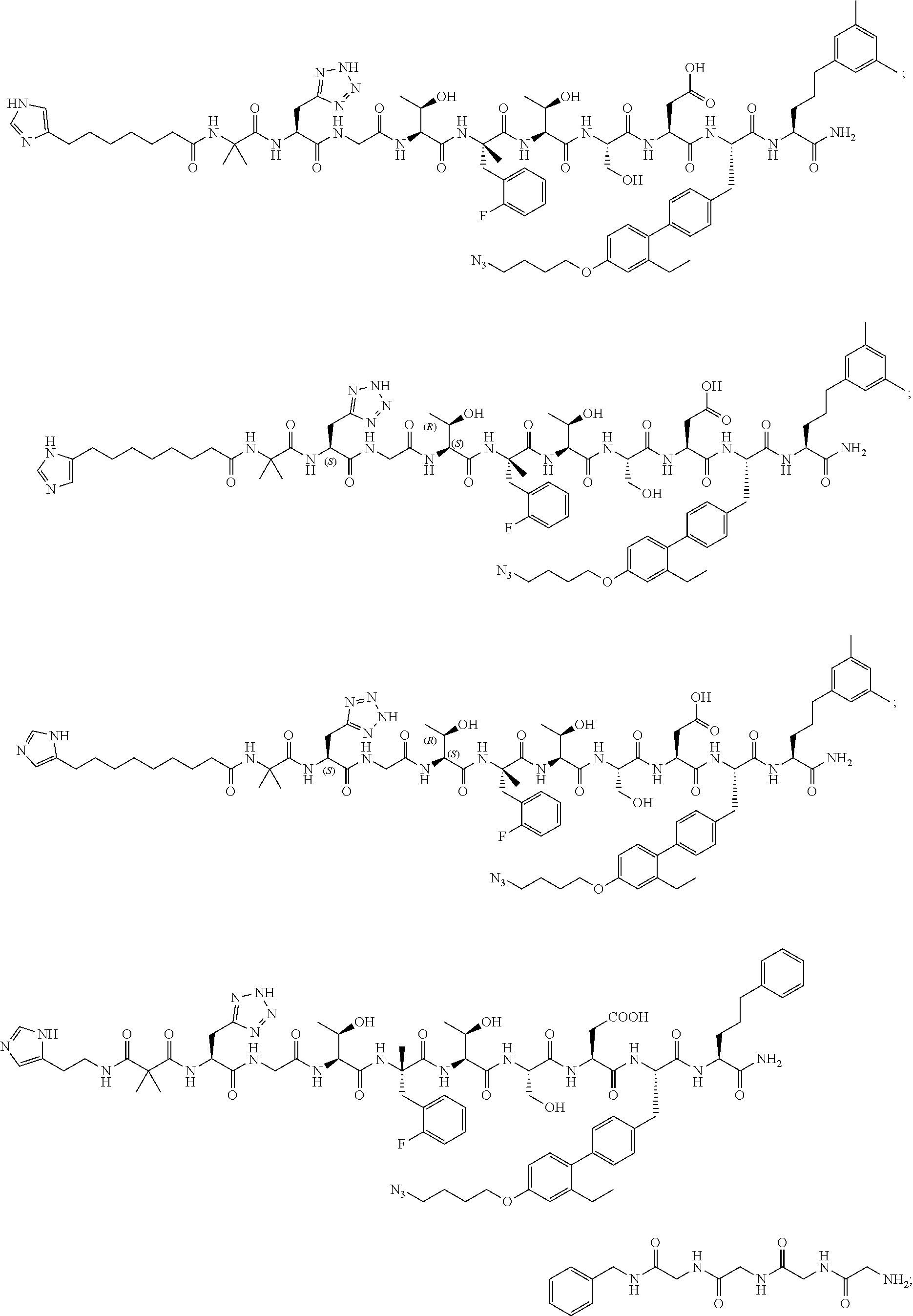

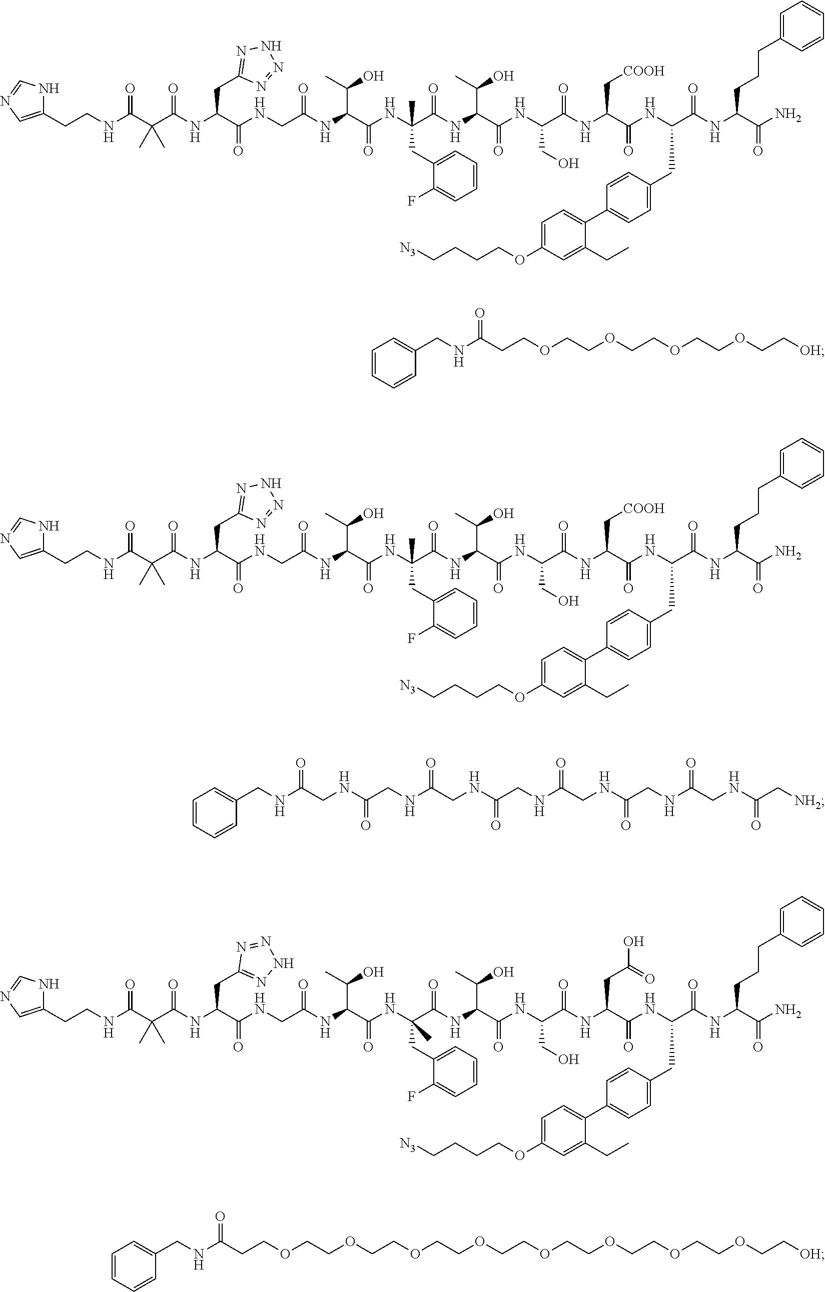

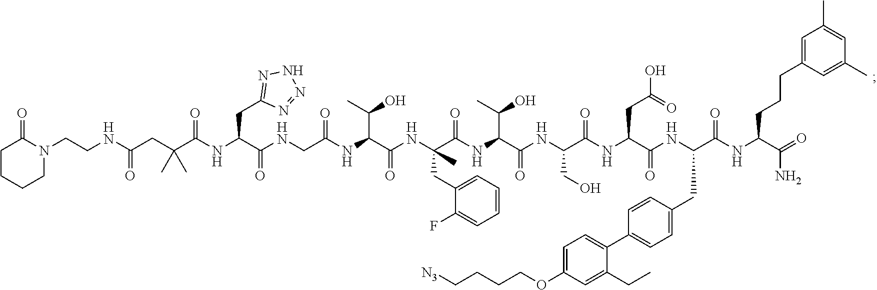 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)CC(=O)NCCN2CCCCC2=O)[C@@H](C)O)[C@@H](C)O)C(=O)N[C@@H](CCCc2cc(C)cc(C)c2)C(N)=O)cc1]

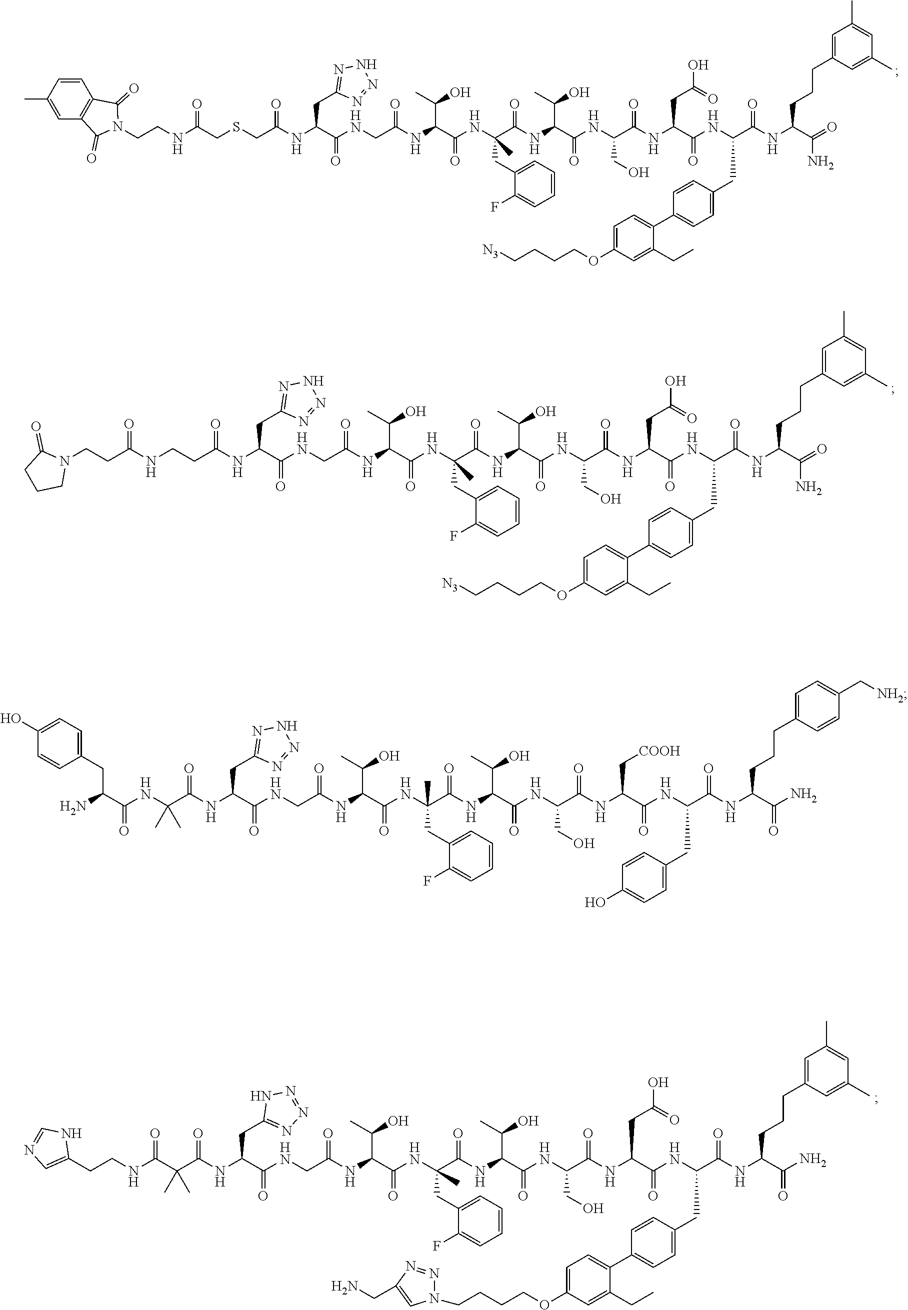

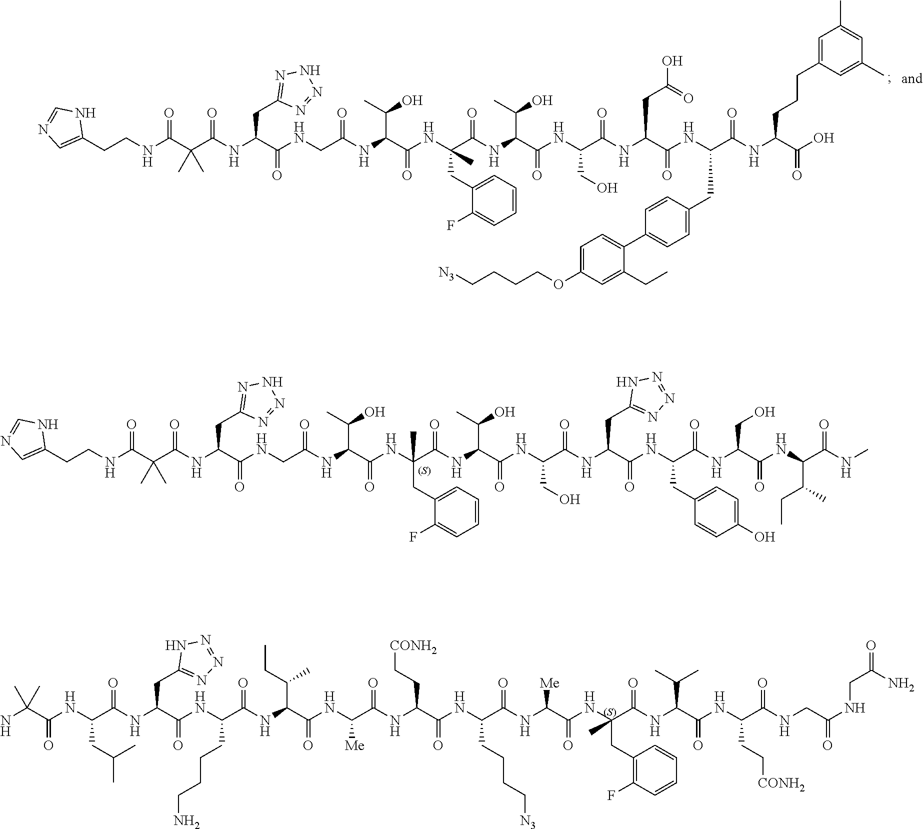

for example, wherein such a P (payload) is conjugated to an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   optionally, wherein the heavy chain immunoglobulin does not comprise a C-terminal lysine or lysine and glycine.

In another aspect of the present invention, provided herein is an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   which is conjugated to a compound having a structure of Formula (C) (SEQ ID NO: 512):

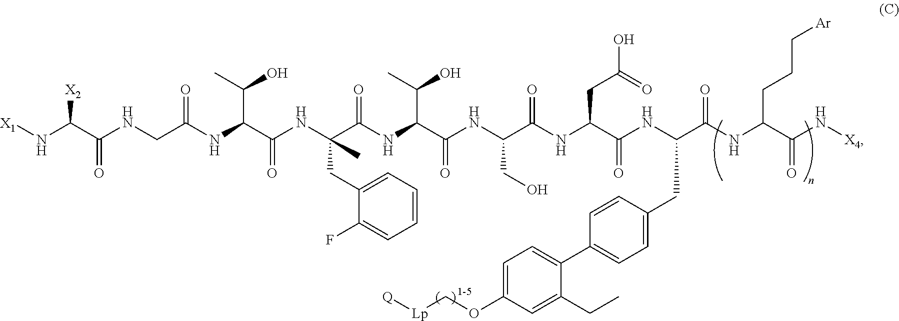

wherein:
Lₚ is absent or a linker comprising one or more of

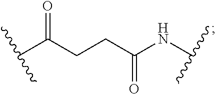 [SMILES: *NC(=O)CCC(*)=O]

a carbamate group; a cyclodextrin; a polyethylene glycol (PEG) segment having 1 to 36 —CH₂CH₂O— (EG) units; a —(CH₂)₂₋₂₄— chain; a triazole; one or more amino acids selected from glycine, serine, glutamic acid, alanine, valine, and proline, and combinations thereof;
Q is a moiety selected from —NH₂, —N₃,

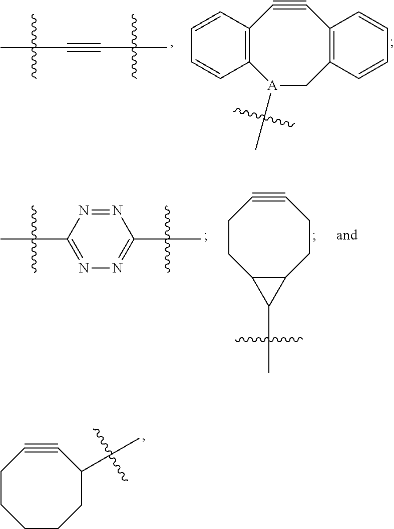

where A is C or N;
X₁ is selected from H;

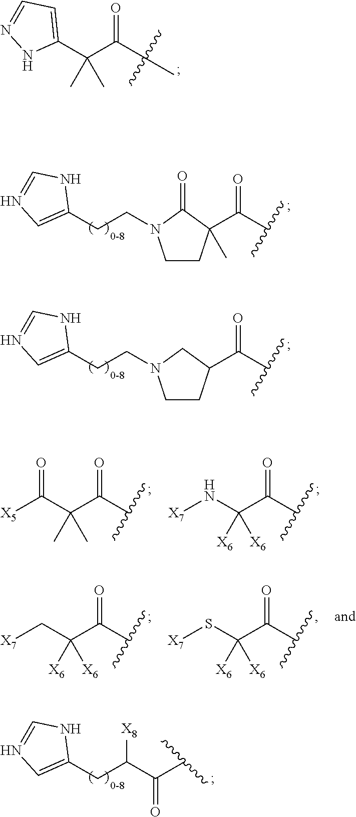

X₂ is selected from

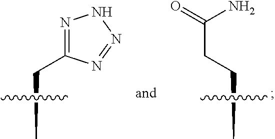

X₃ is selected from —CH₃, —(CH₂)₂₋₆—NH₂, —(CH₂)₂₋₆—N₃, and —(CH₂)₂₋₆-Tr-(CH₂)₁₋₆—NH₂, where Tr is a triazole moiety;
n is 0 or 1;
X₄ is selected from —NH₂, —OH and —N(H)(phenyl);
X₅ is selected from —OH, —NH₂, —NH—OH, and

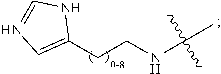

X₆ is independently at each occurrence selected from H, —OH, —CH₃, and —CH₂OH;
X₇ is selected from H,

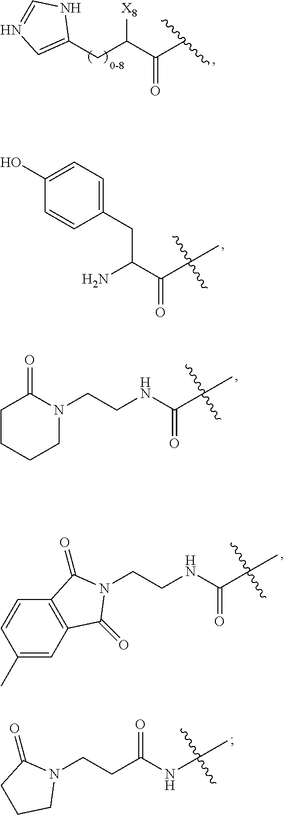

X₈ is selected from H, —OH, —NH₂, and

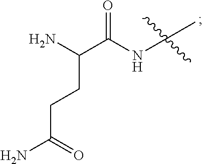 [SMILES: *NC(=O)C(N)CCC(N)=O]

Ar is selected from

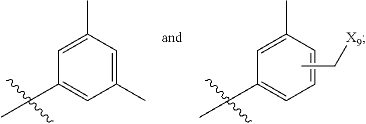

X₉ is selected from —NH₂

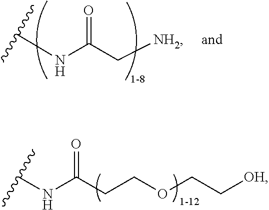

m is an integer from 1 to 4
or a pharmaceutically acceptable salt thereof;
optionally, wherein the heavy chain immunoglobulin does not comprise a C-terminal lysine or lysine and glycine.

In another aspect, provided herein is an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i); which is conjugated to a compound having a structure of Formula (III) (SEQ ID NO: 513):

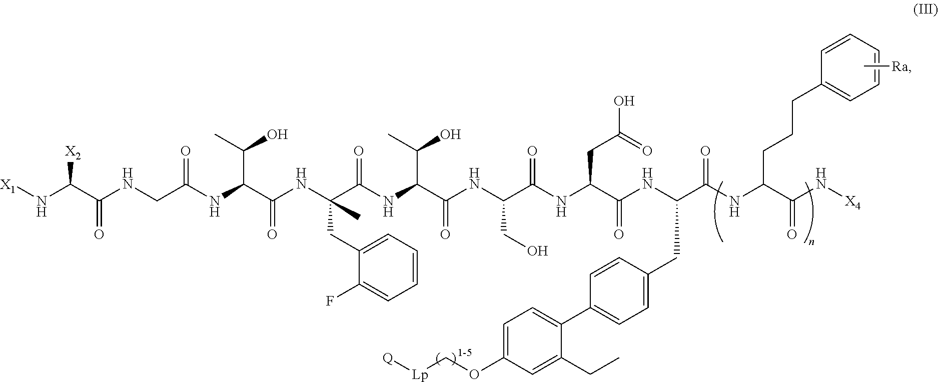

wherein:
Lₚ is absent or a linker comprising one or more of

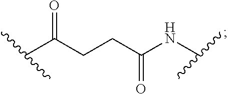 [SMILES: *NC(=O)CCC(*)=O]

a carbamate group; a cyclodextrin; a polyethylene glycol (PEG) segment having 1 to 36 —CH₂CH₂O— (EG) units; one or more amino acids selected from glycine, serine, glutamic acid, alanine, valine, and proline, and combinations thereof;
Q is a moiety selected from —N₃

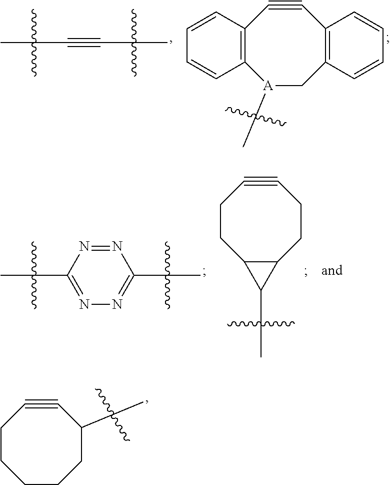

where A is C or N;
X₁ is selected from H;

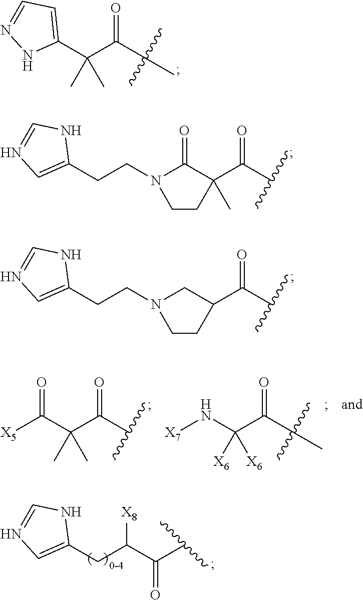

X₂ is selected from

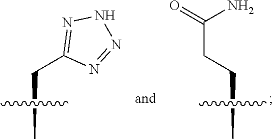

X₃ is selected from —(CH₂)₂₋₆—NH₂, —(CH₂)₂₋₆—N₃, and —CH₃, with the proviso that when X₃ is —CH₃, n is 1 and Ra in at least one occurrence is selected from —(CH₂)₂₋₆—NH₂ and —(CH₂)₂₋₆—N₃;
n is 0 or 1;
Ra is independently at each occurrence selected from H, —CH₃, —(CH₂)₂₋₆—NH₂, and —(CH₂)₂₋₆—N₃;
X₄ is selected from H and phenyl;
X₅ is selected from —OH, —NH₂, —NH—OH, and

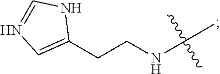 [SMILES: *NCCc1cnc[nH]1]

X₆ is independently at each occurrence selected from H, —OH, —CH₃, and —CH₂OH;
X₇ is selected from H,

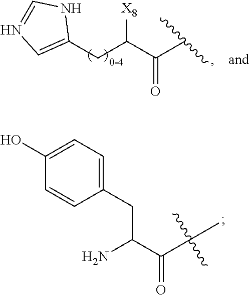

X₈ is selected from H, —OH, —NH₂, and

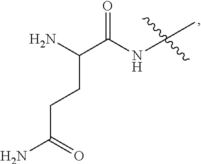 [SMILES: *NC(=O)C(N)CCC(N)=O]

and pharmaceutically acceptable salts thereof;
optionally, wherein the heavy chain immunoglobulin does not comprise a C-terminal lysine or lysine and glycine.

The present invention provides an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   that is conjugated to a compound having a structure selected from the group consisting of (SEQ ID NOS 514, 514, 514, 514, 514-519, 519, 519-532, 515-516, 534-536, 538, 536-537, 521, 539-541, 541-543, 519, 544 and 544-566, respectively, in order of appearance):

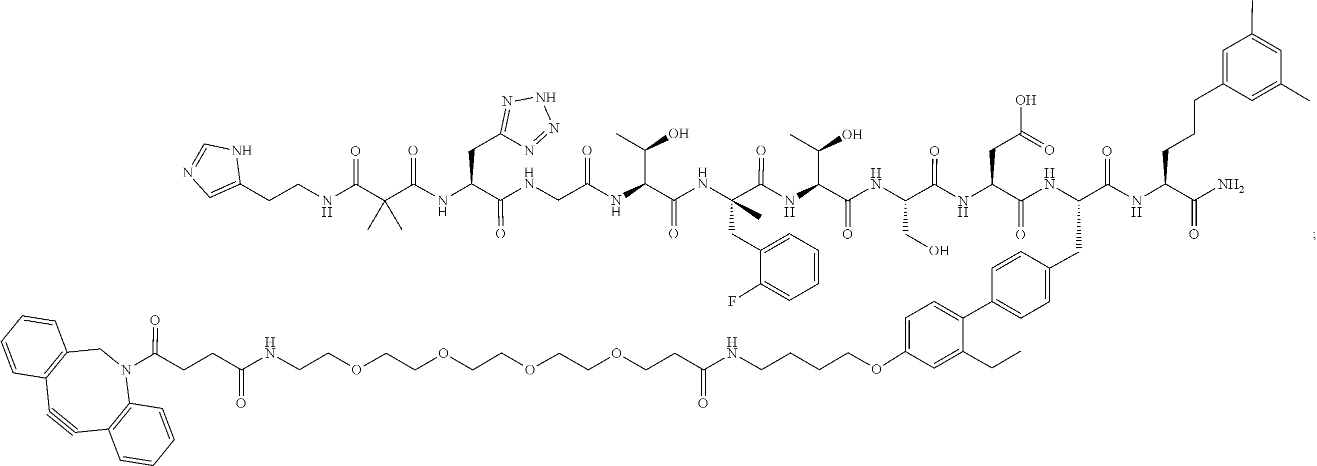 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

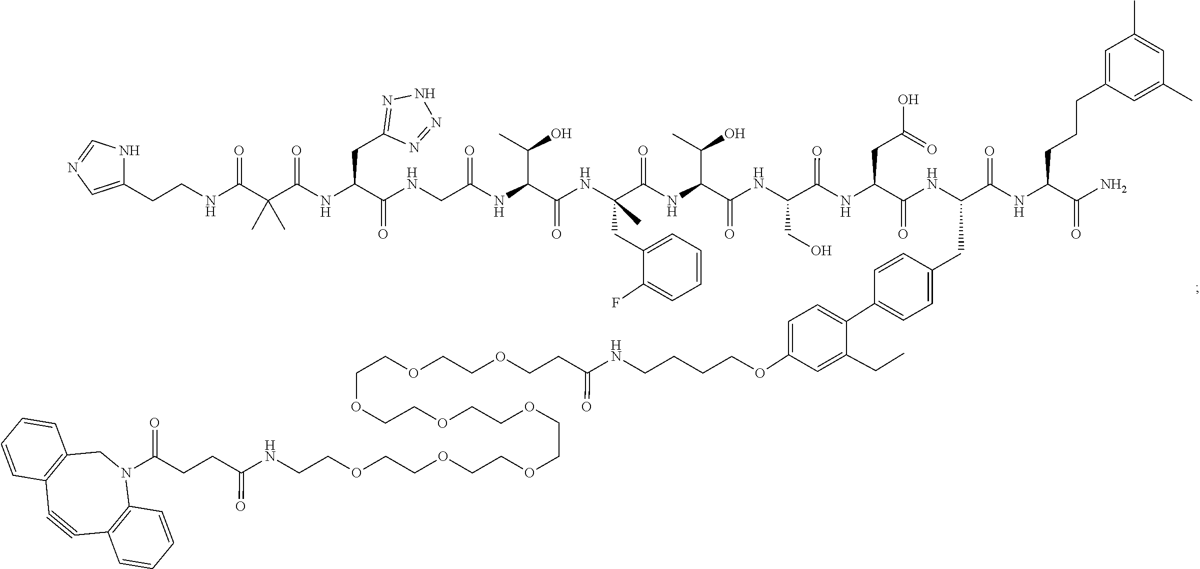 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

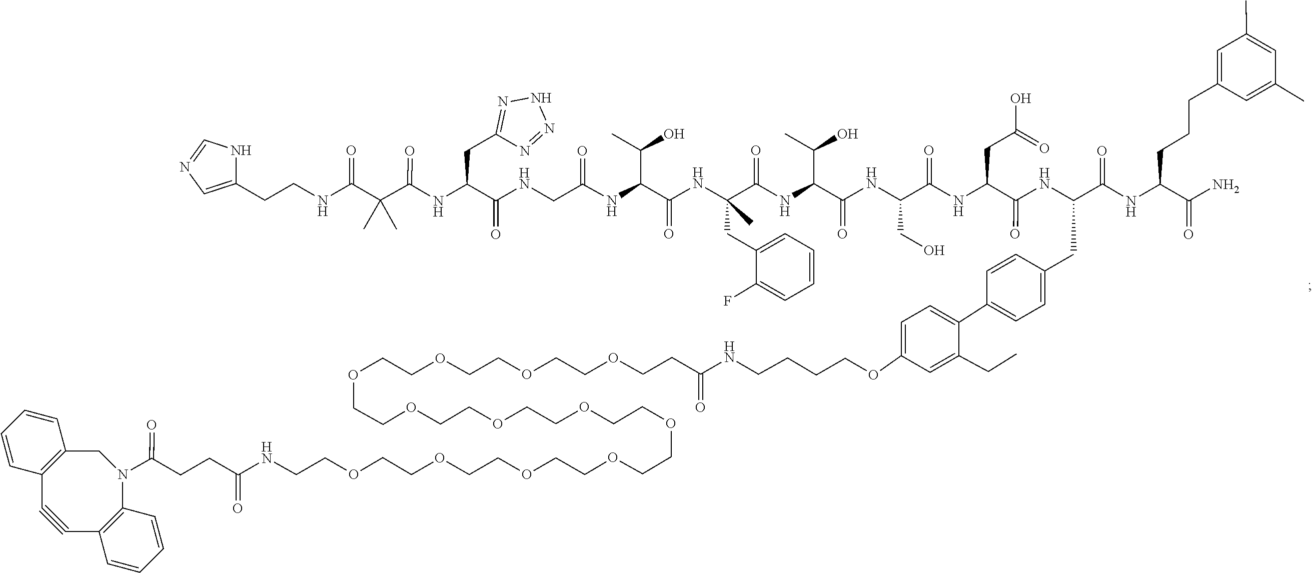 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

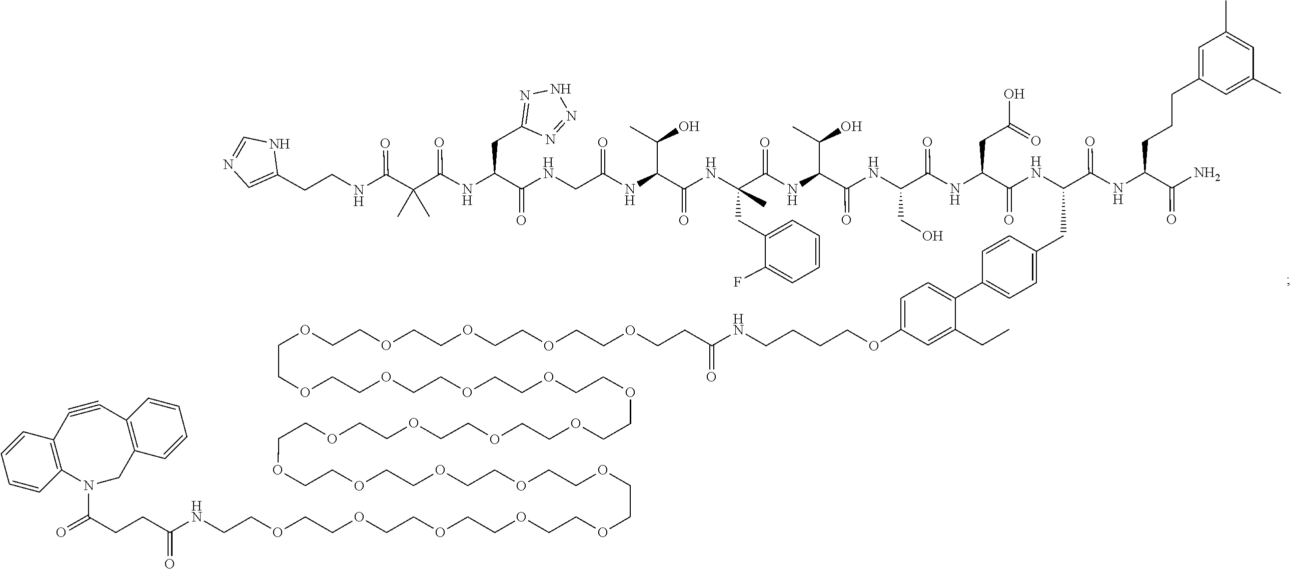 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

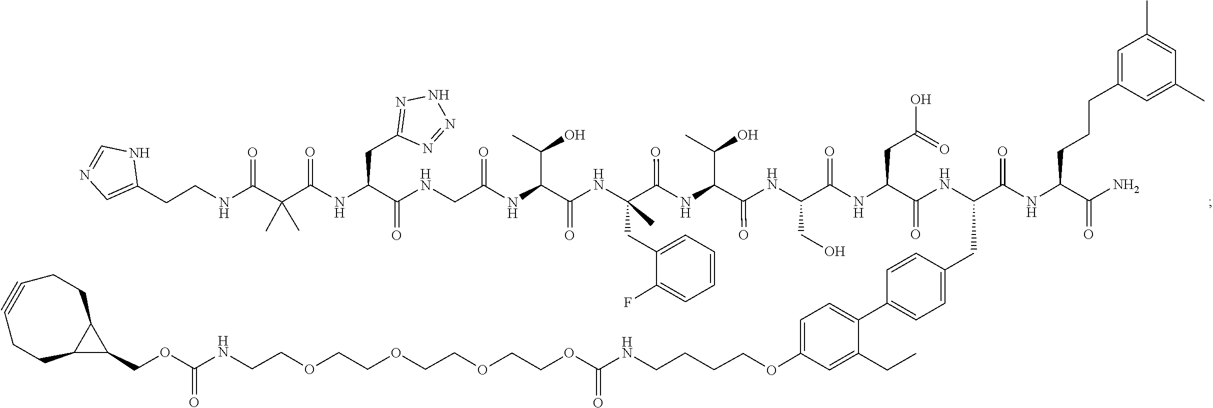 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

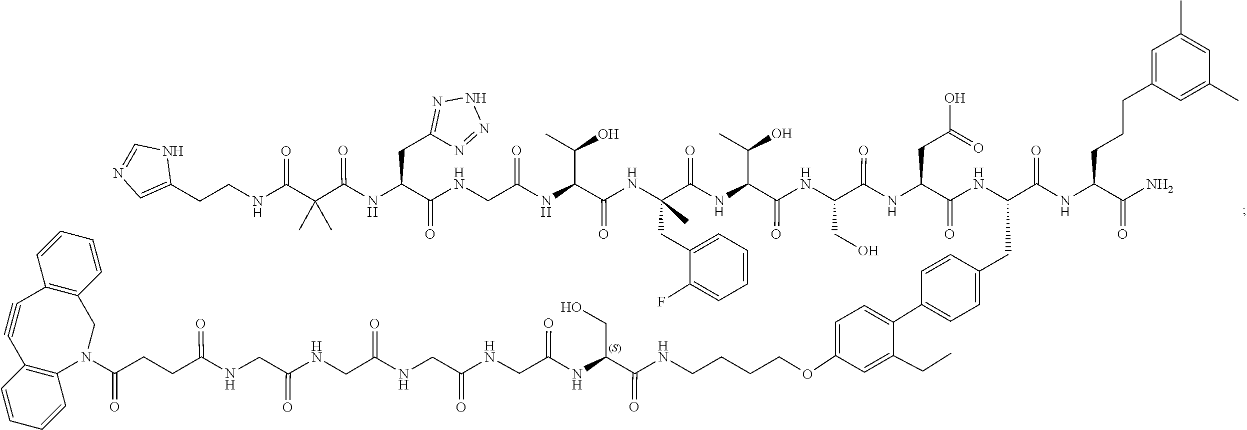

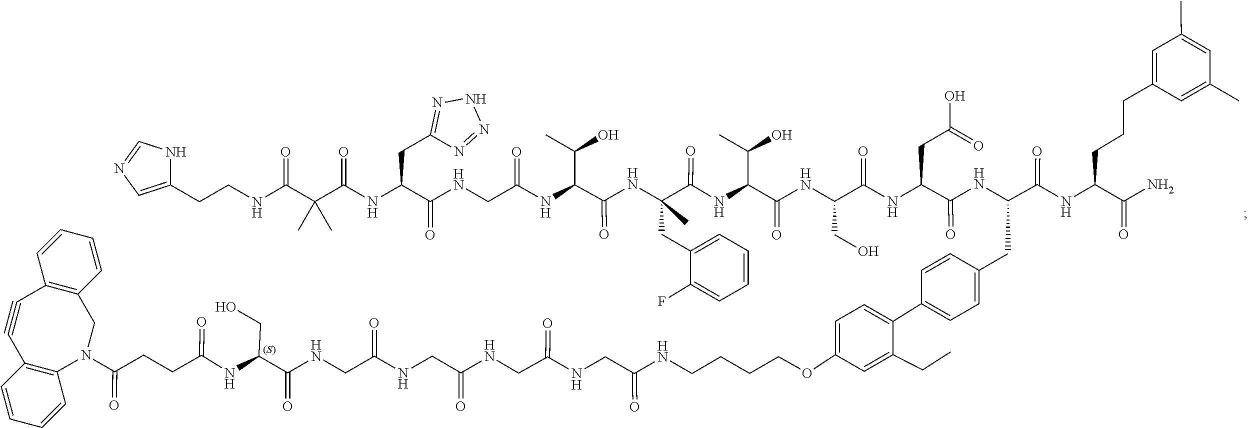

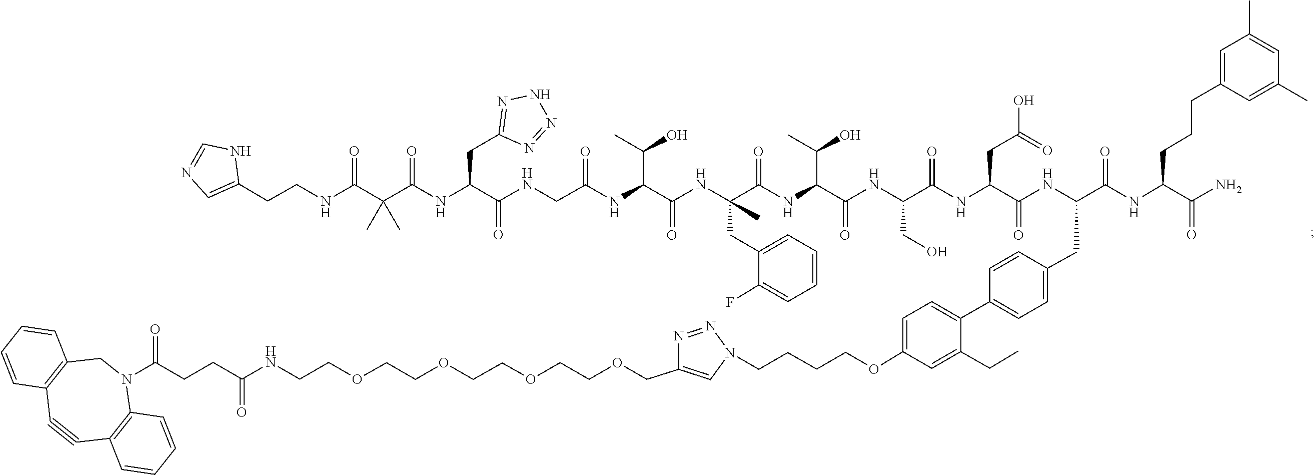 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

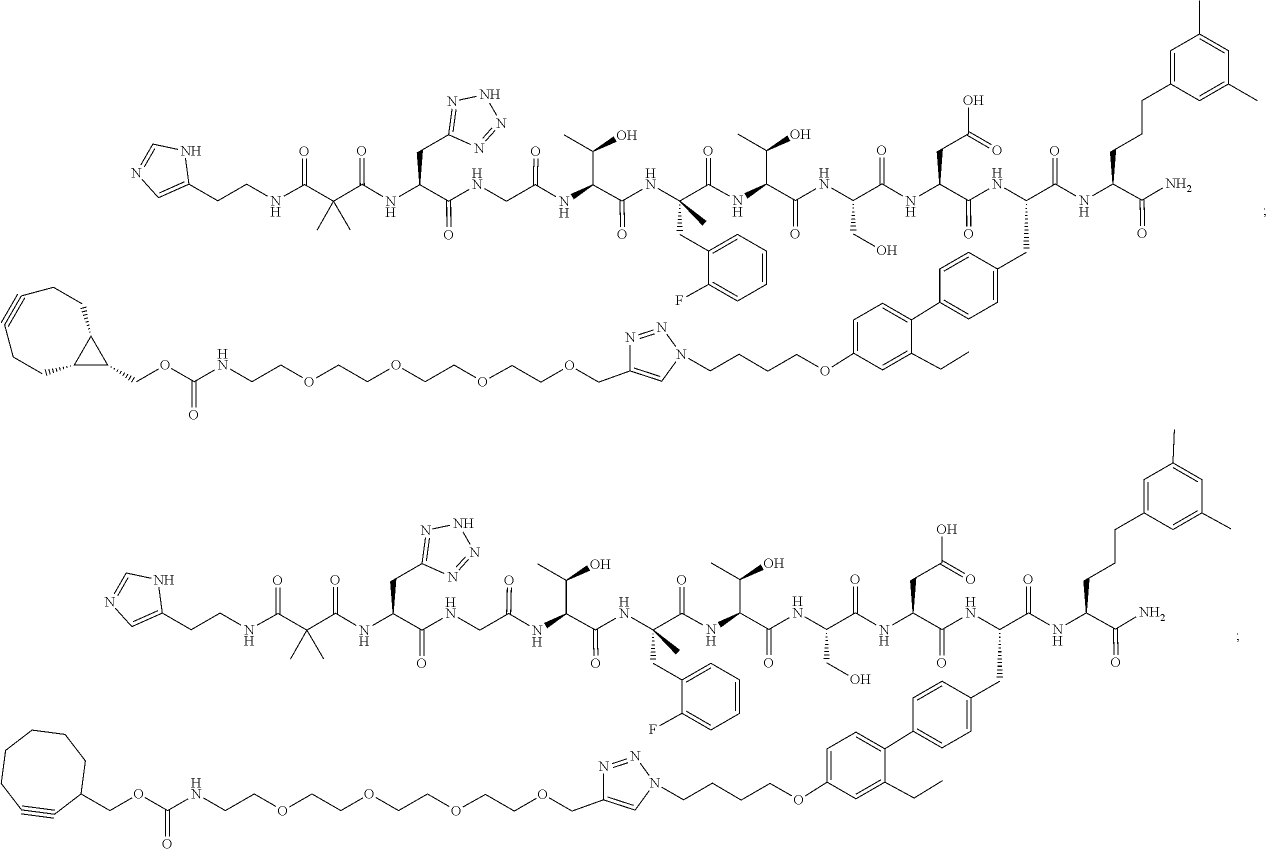

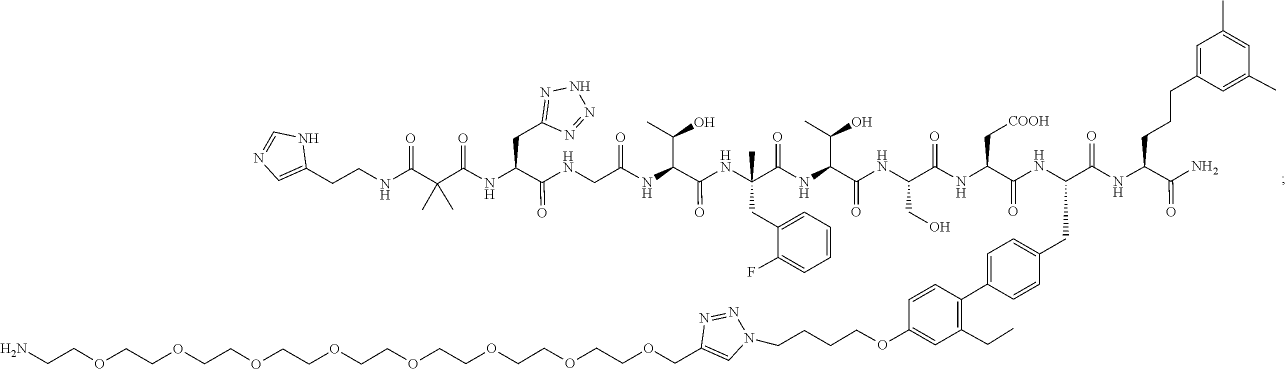

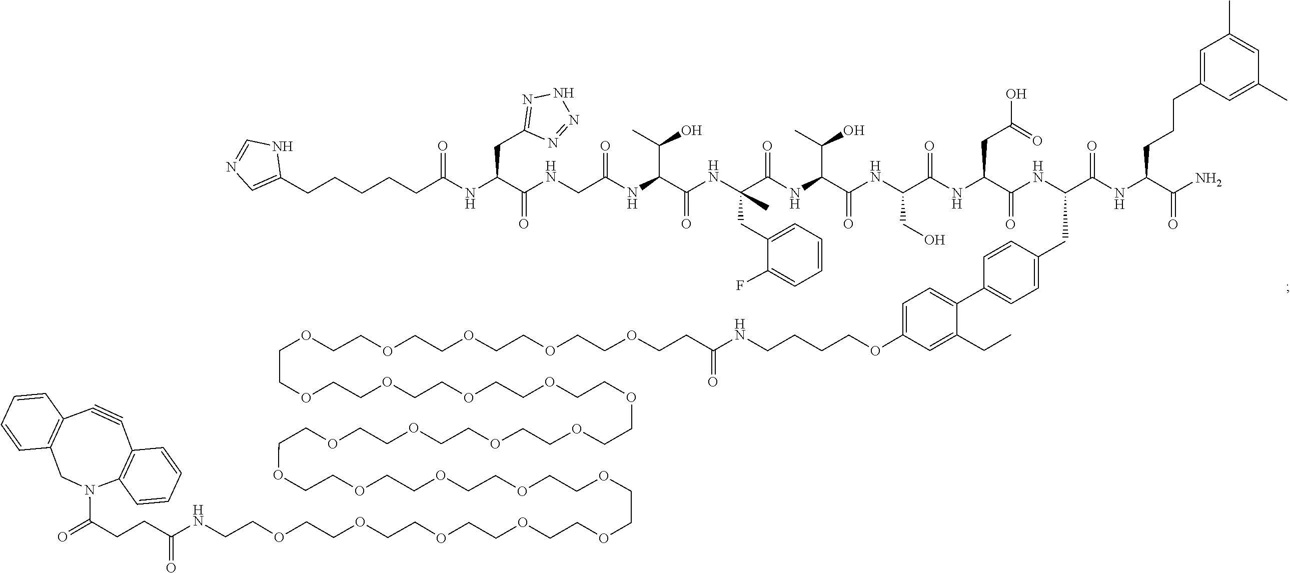 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(N)=O)cc1]

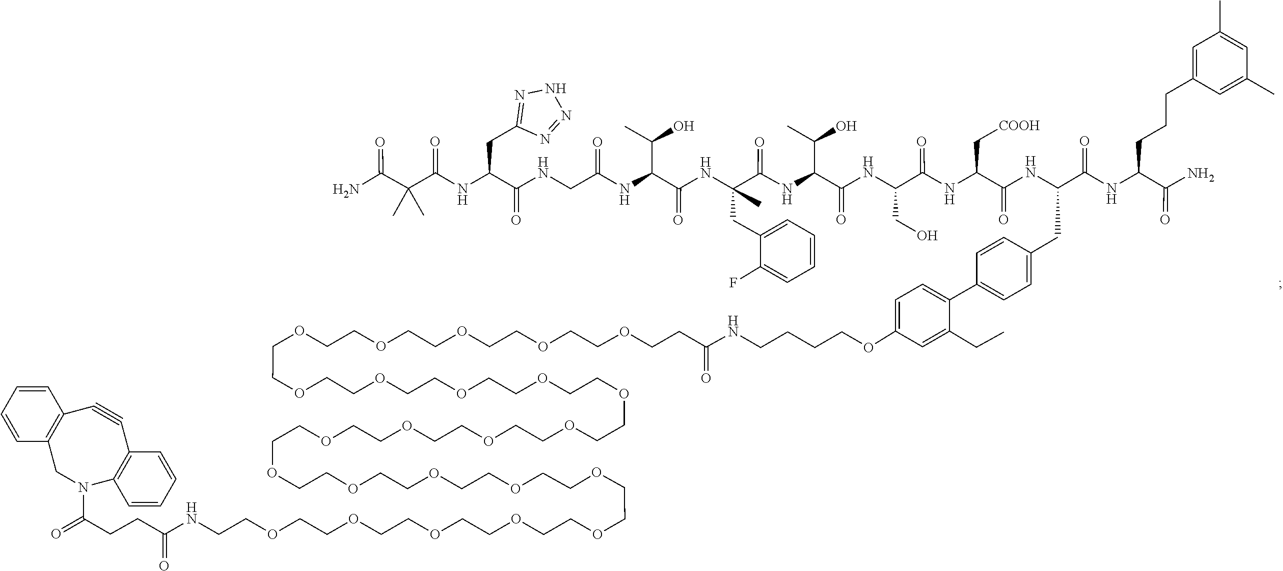 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(N)=O)cc1]

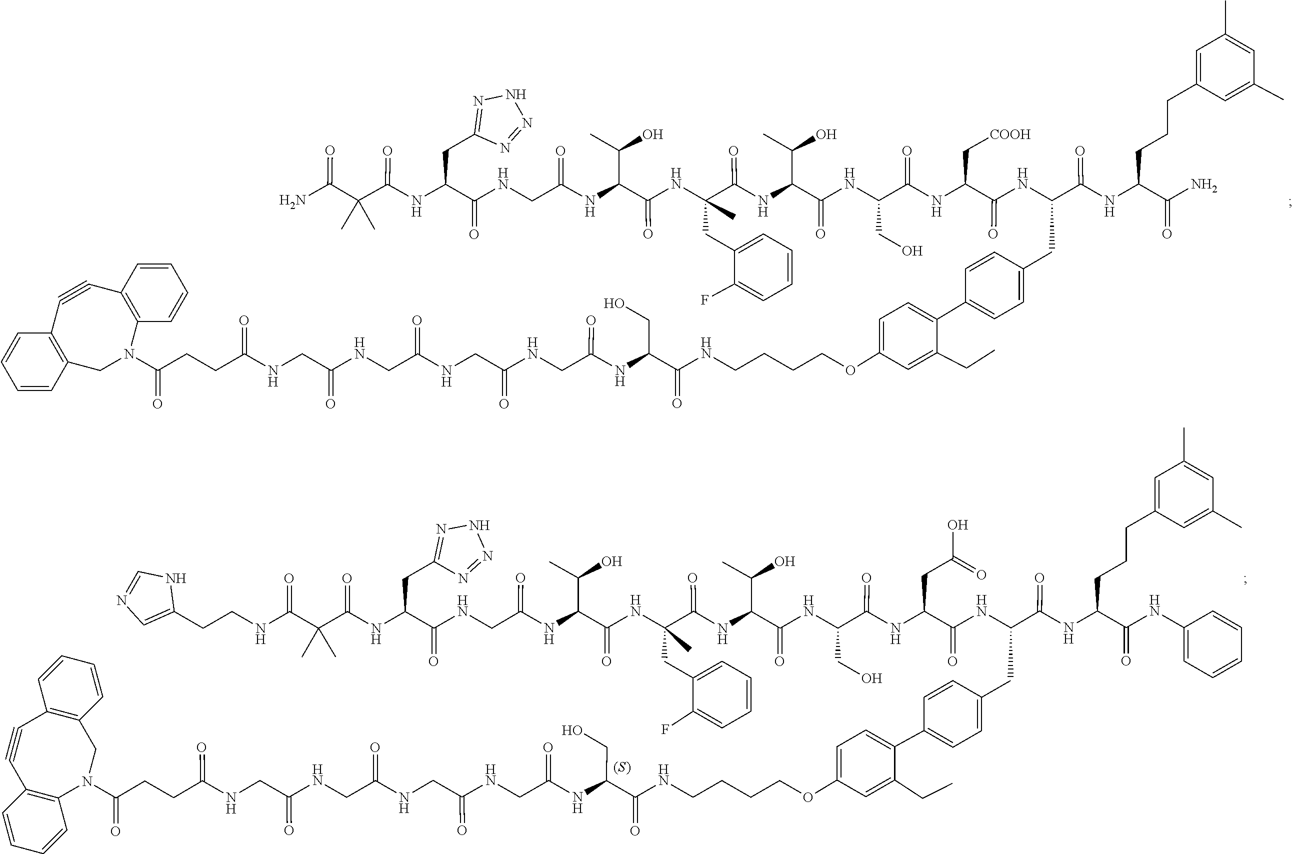

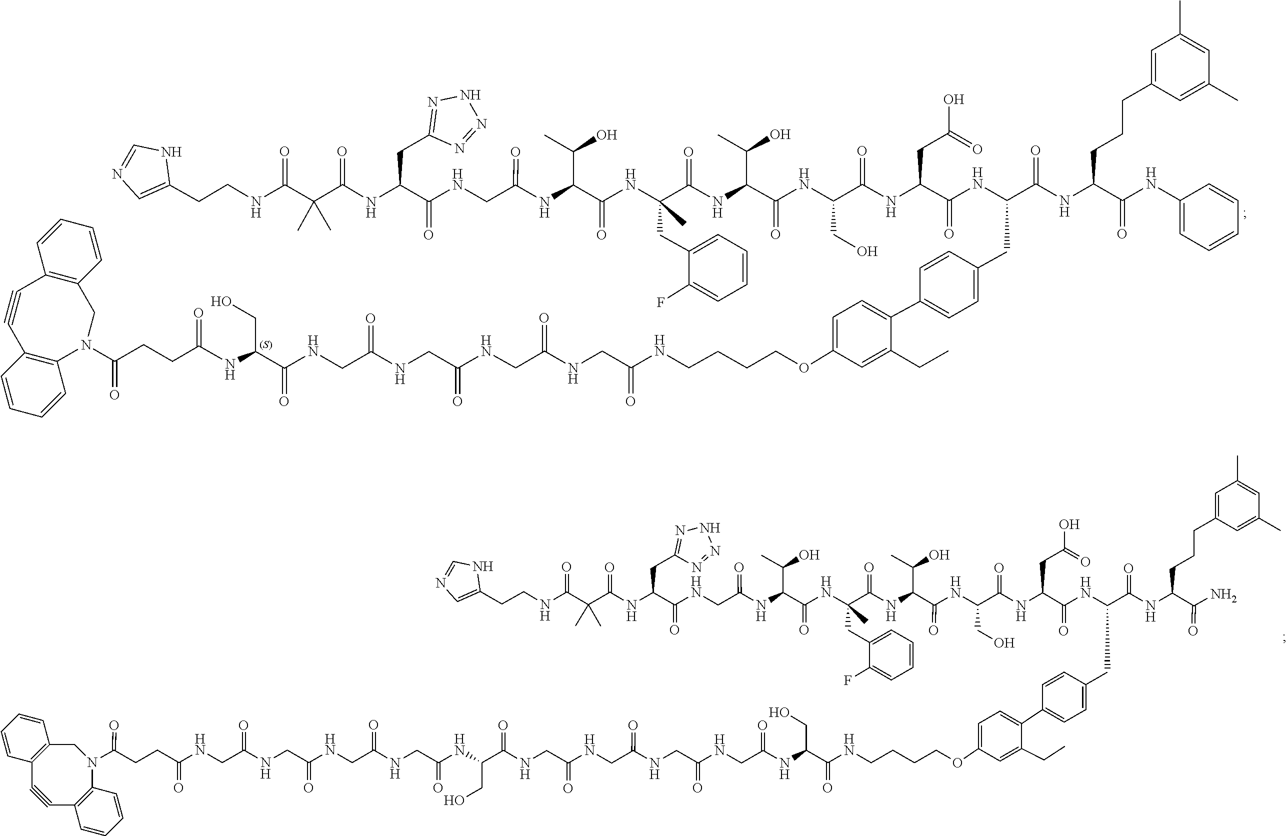

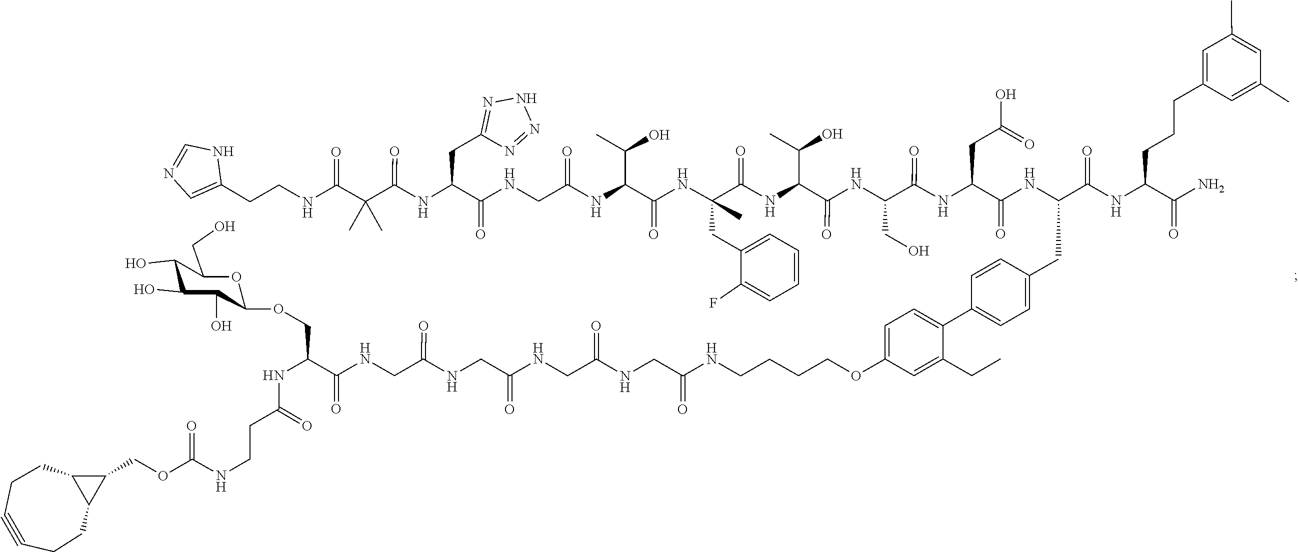

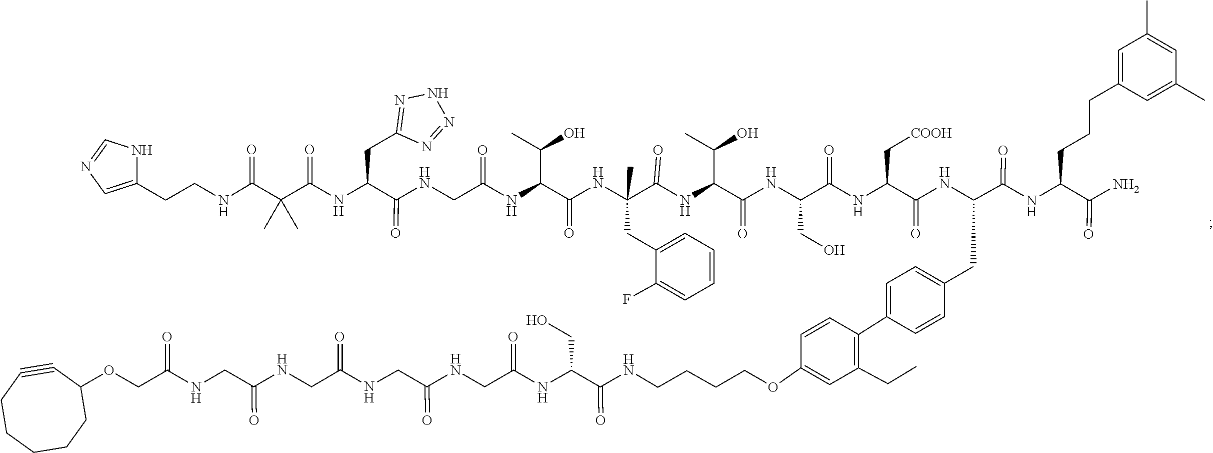

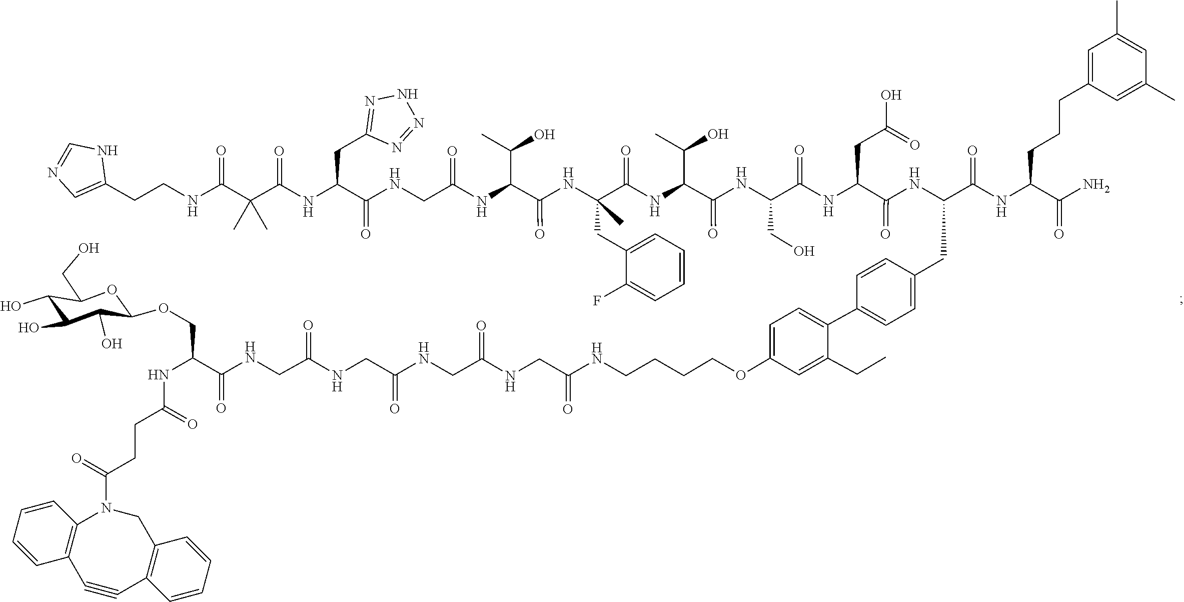

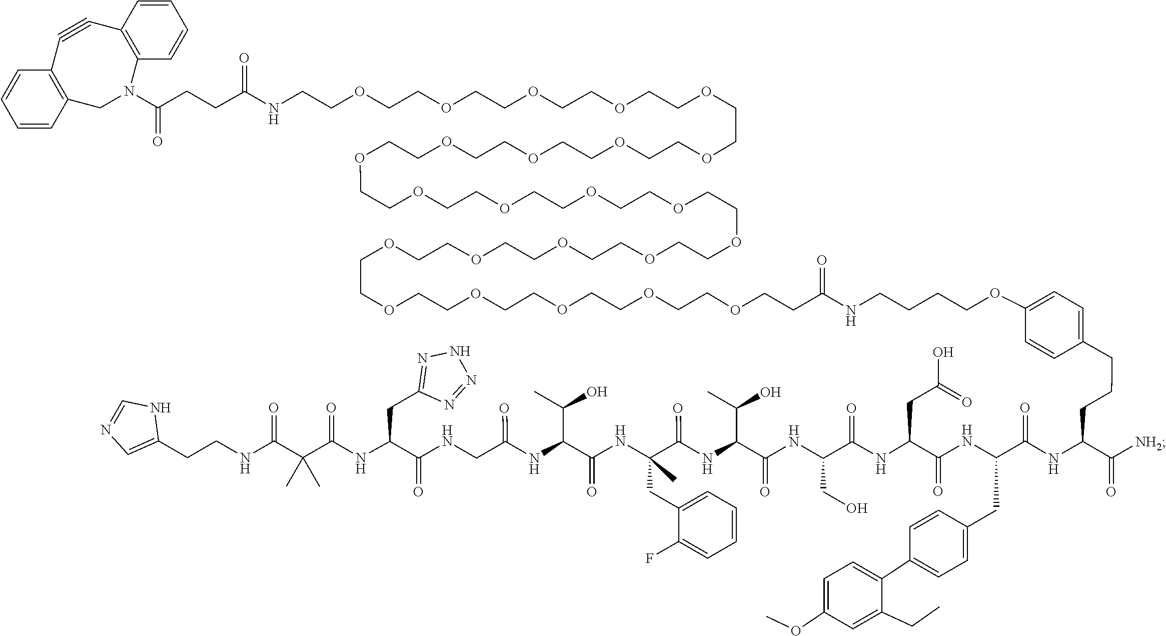 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4C#Cc4ccccc43)cc2)C(N)=O)cc1]

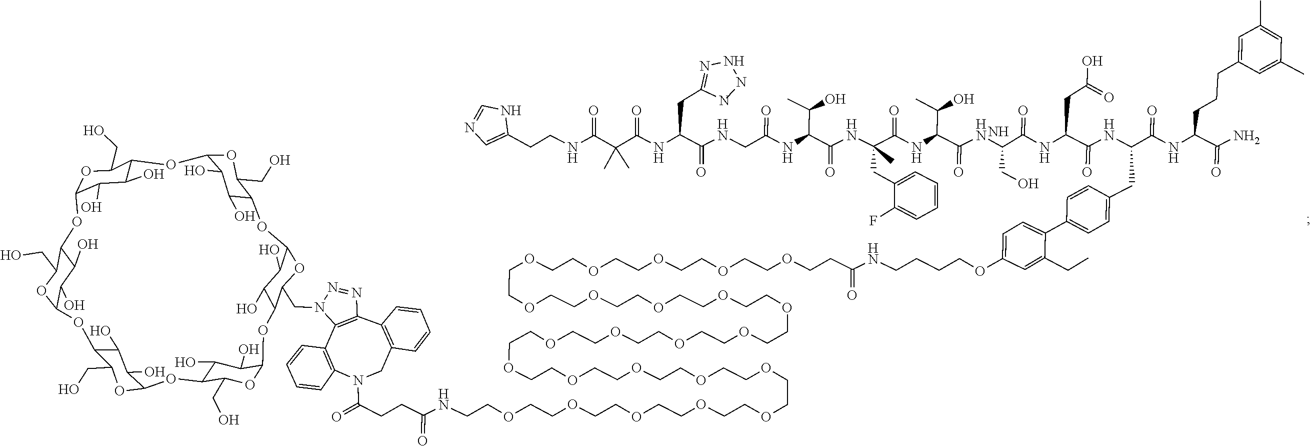

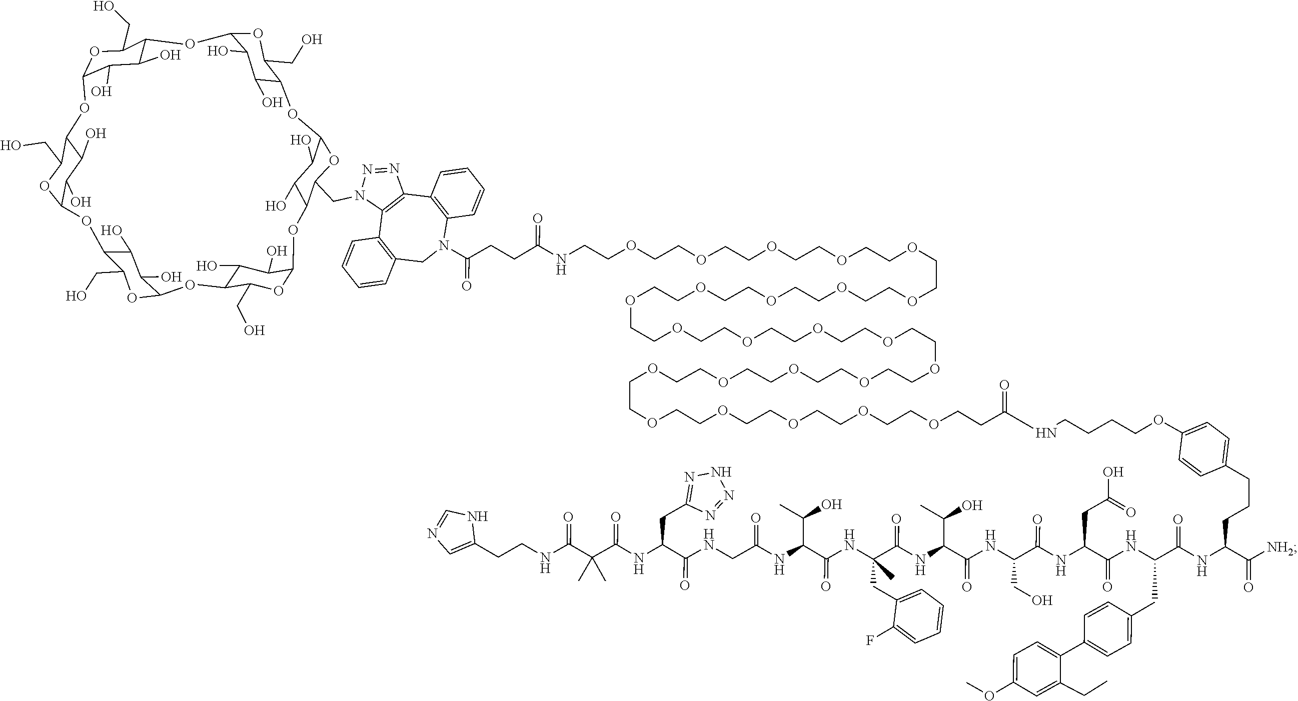 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4-c4c(nnn4CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)-c4ccccc43)cc2)C(N)=O)cc1]

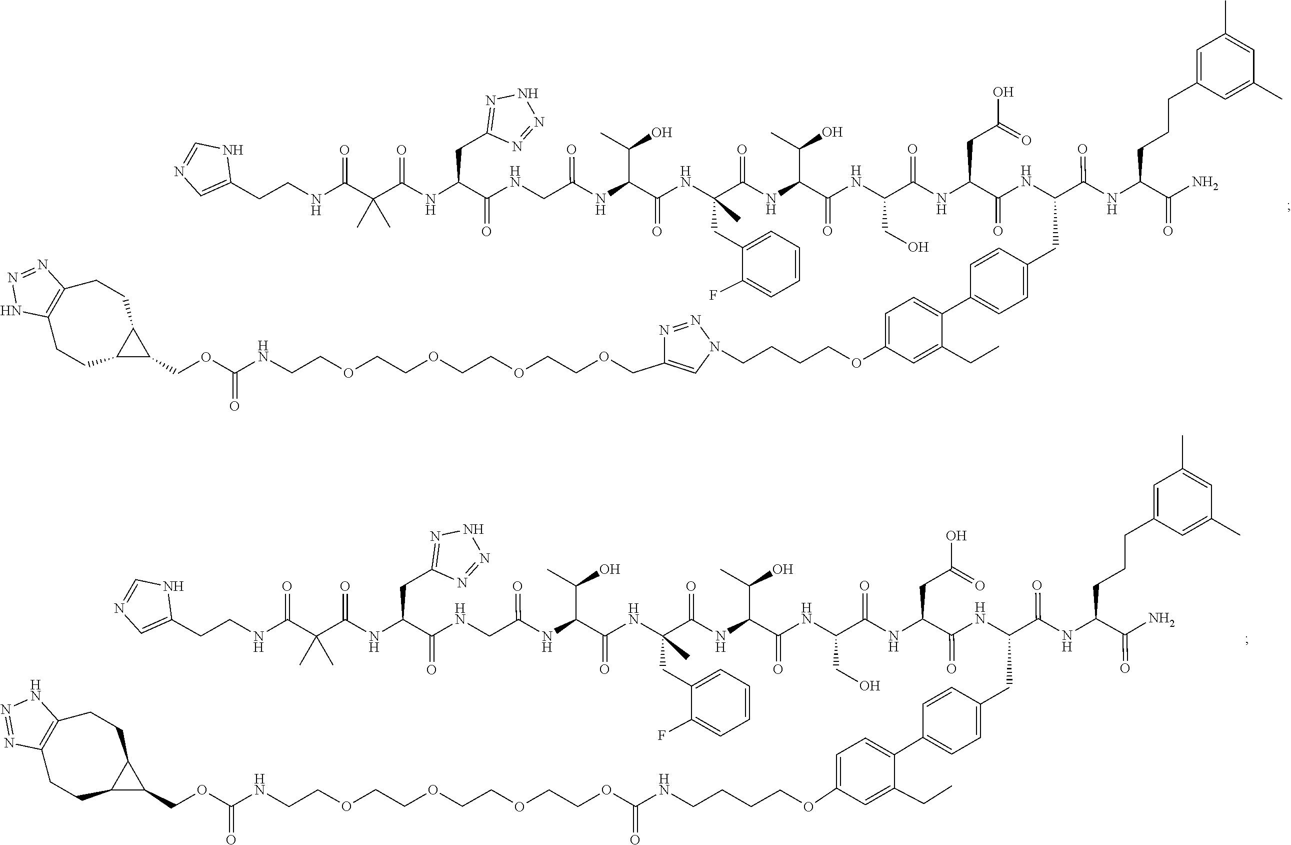

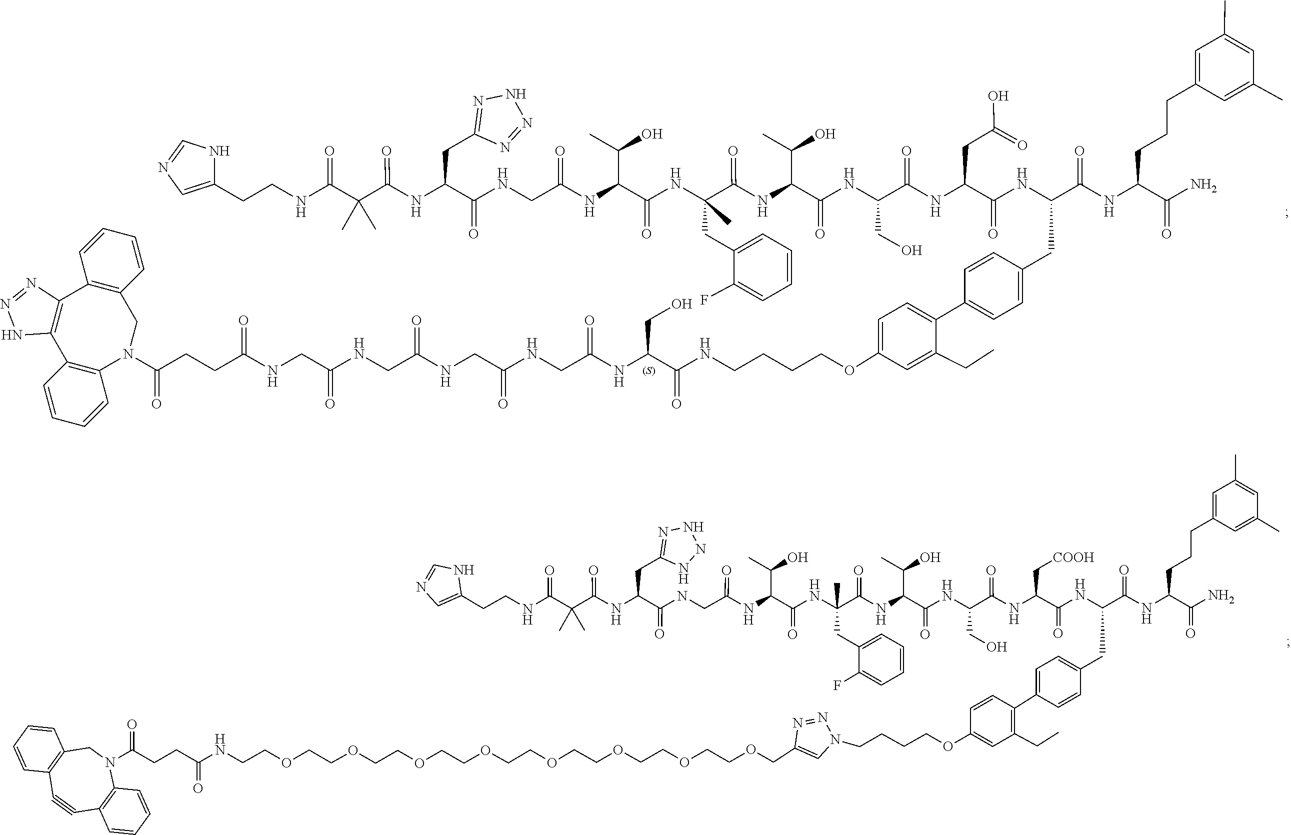

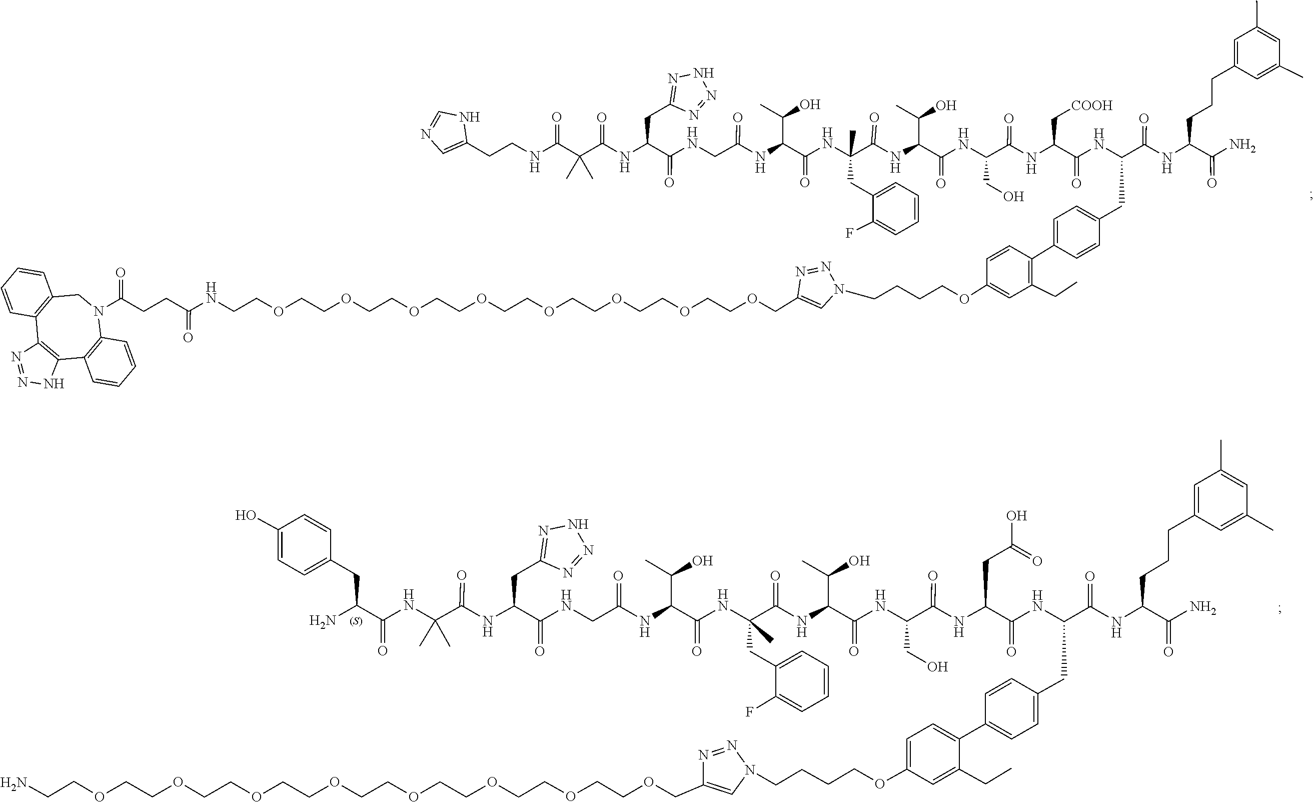

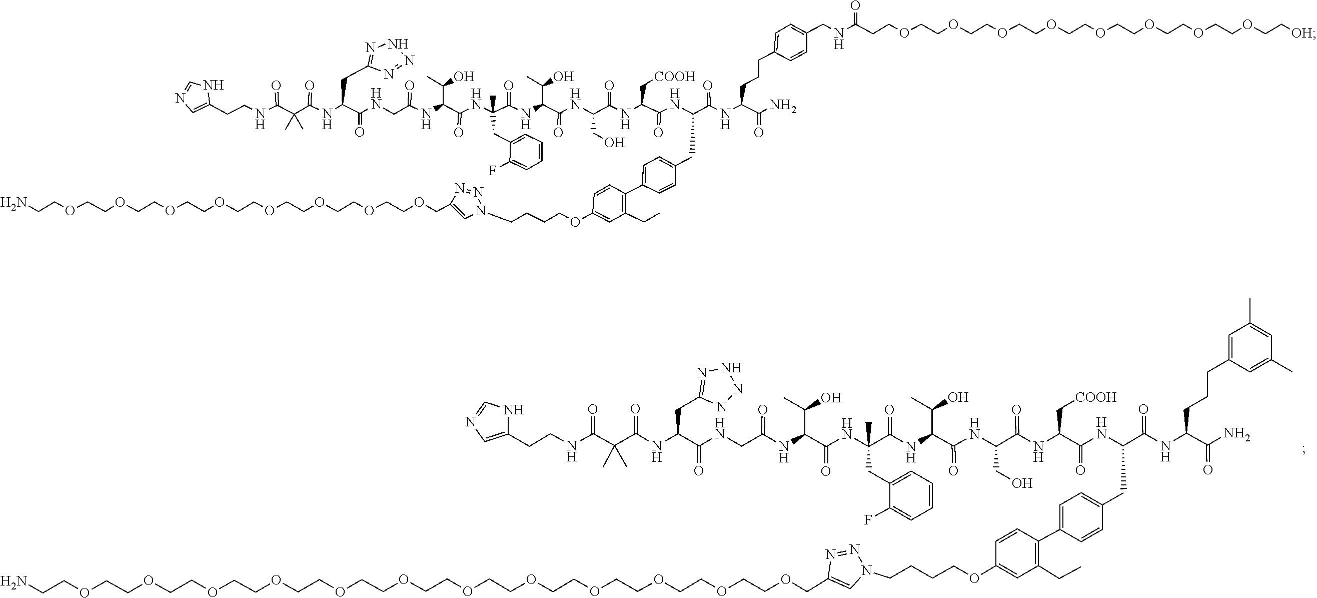

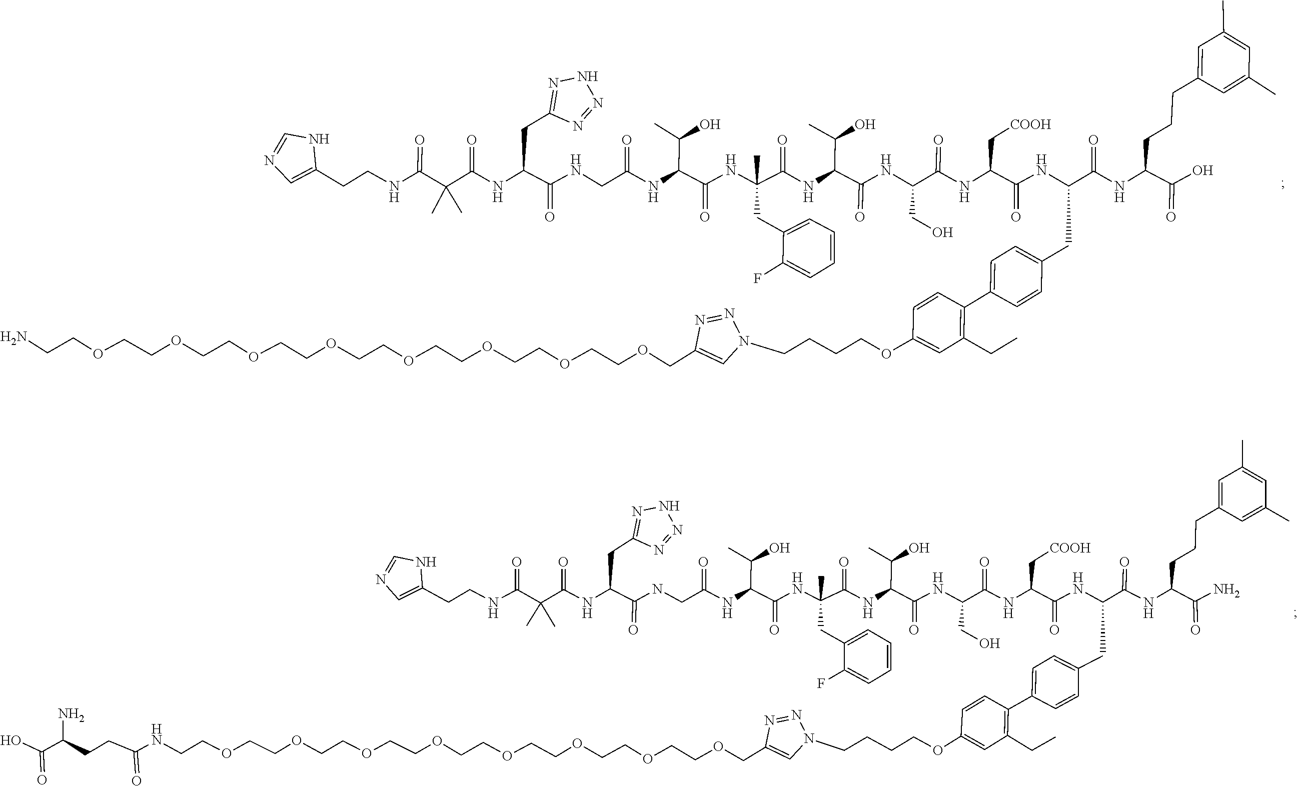

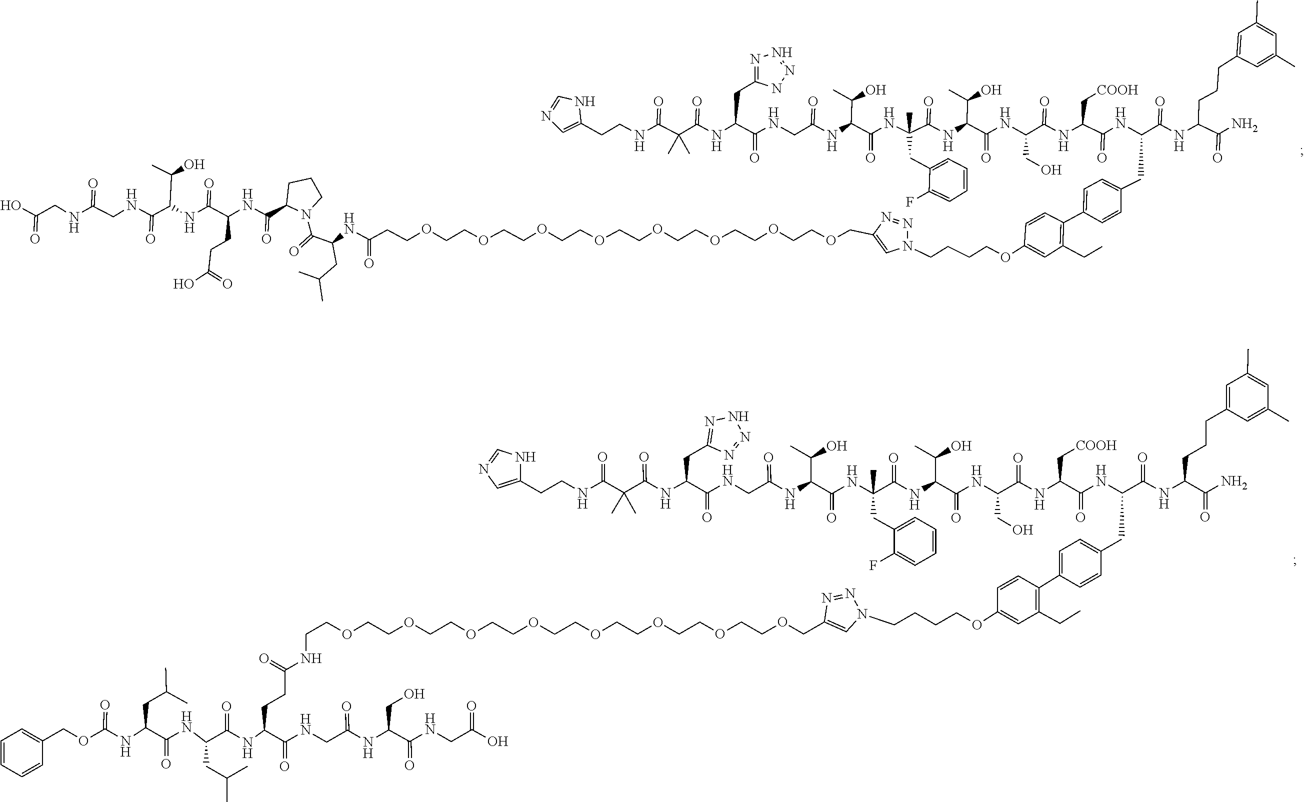

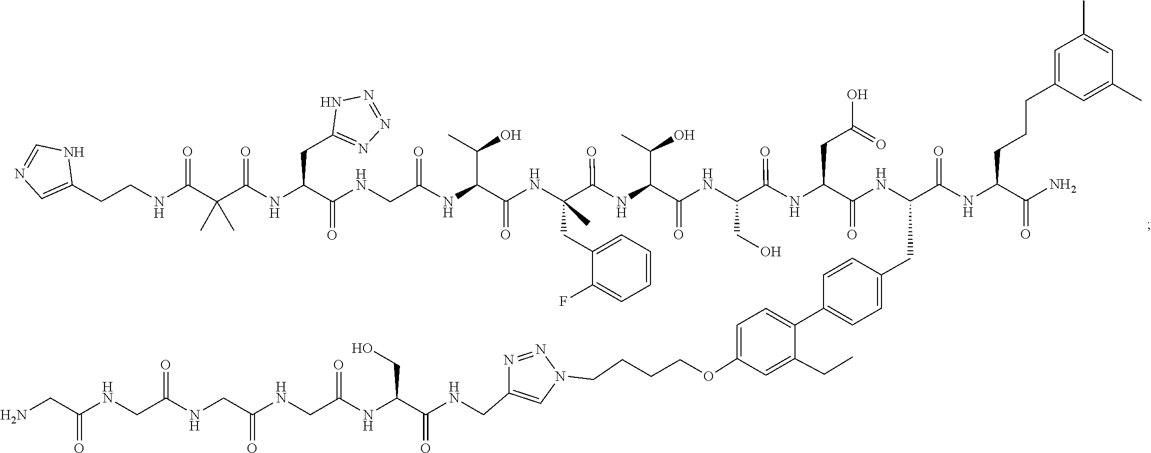 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1]

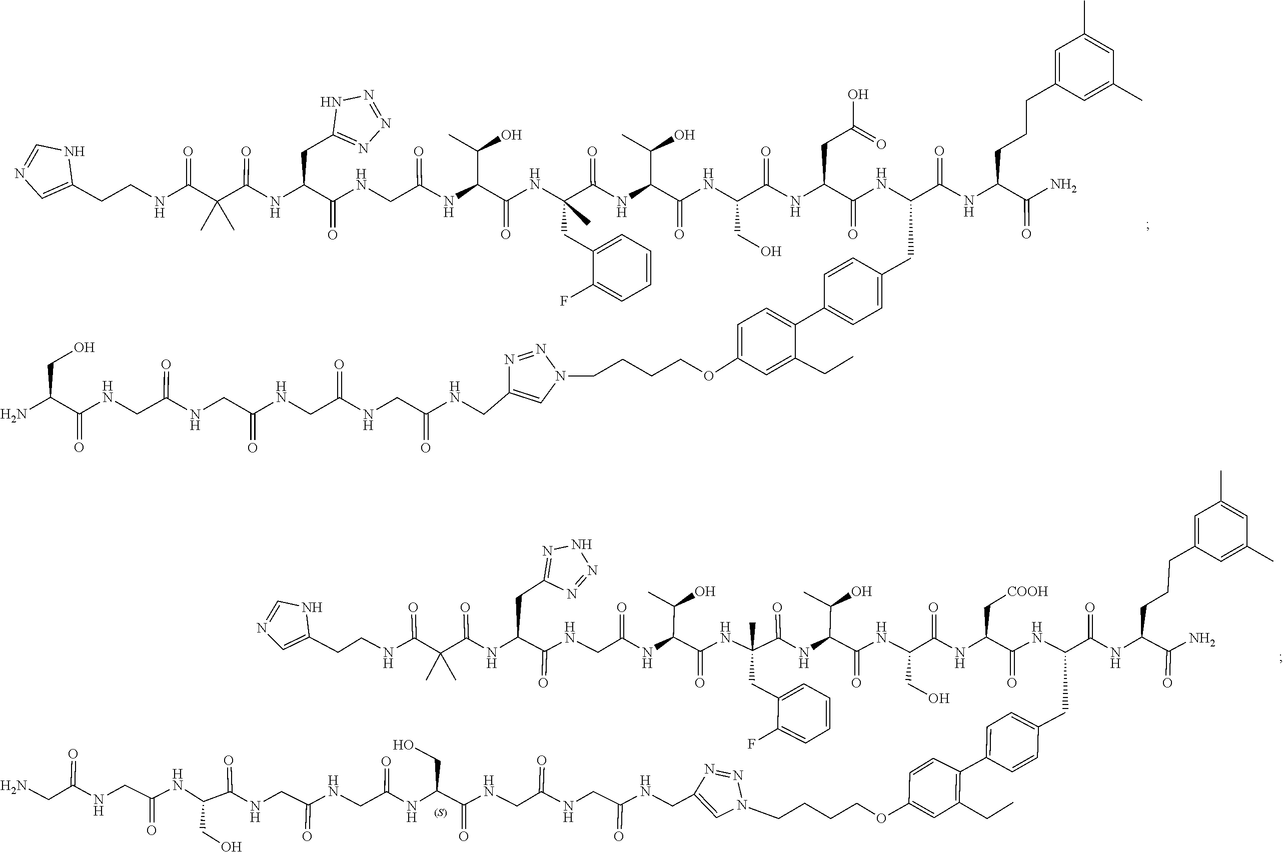

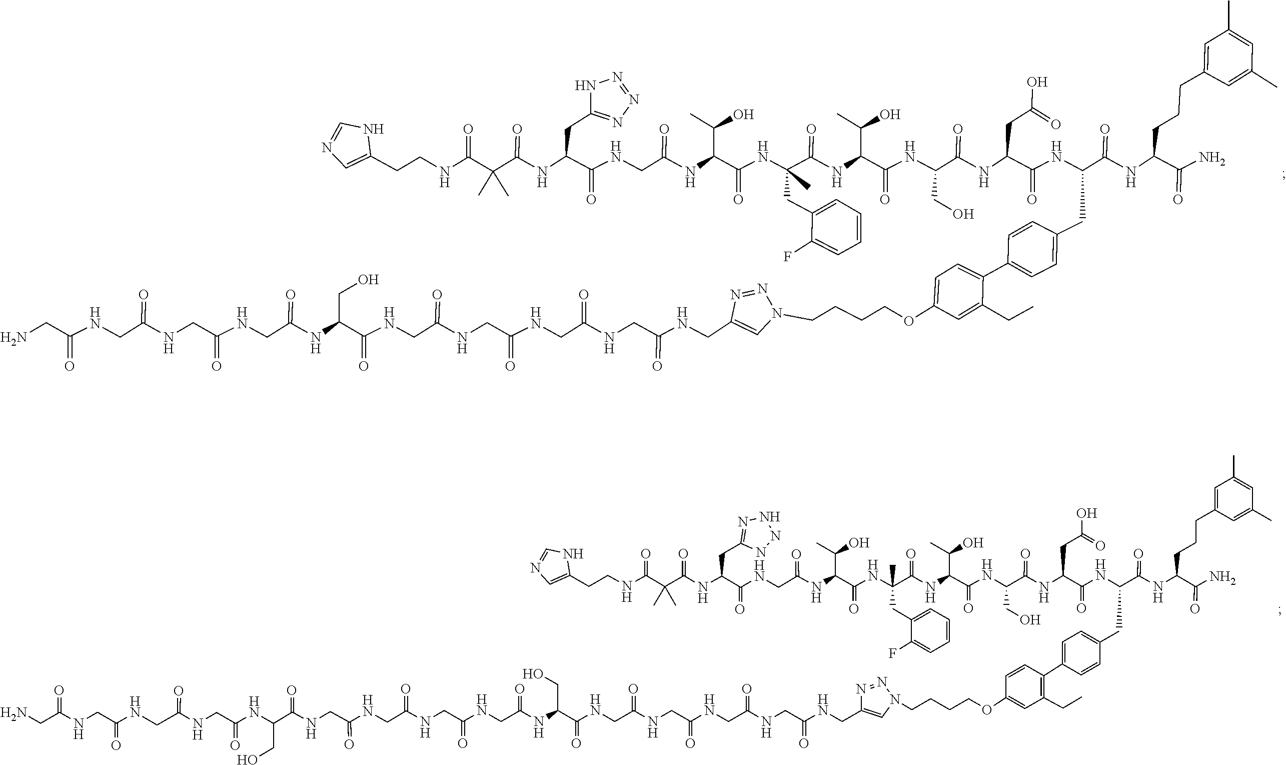

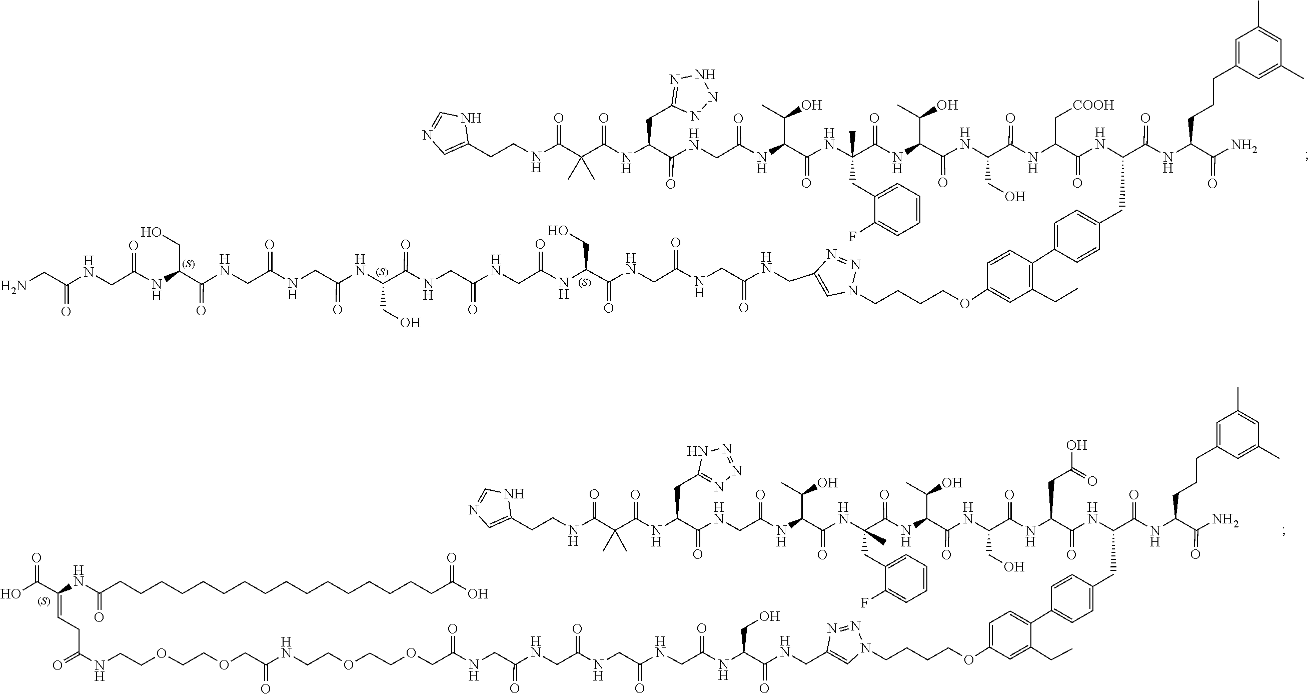

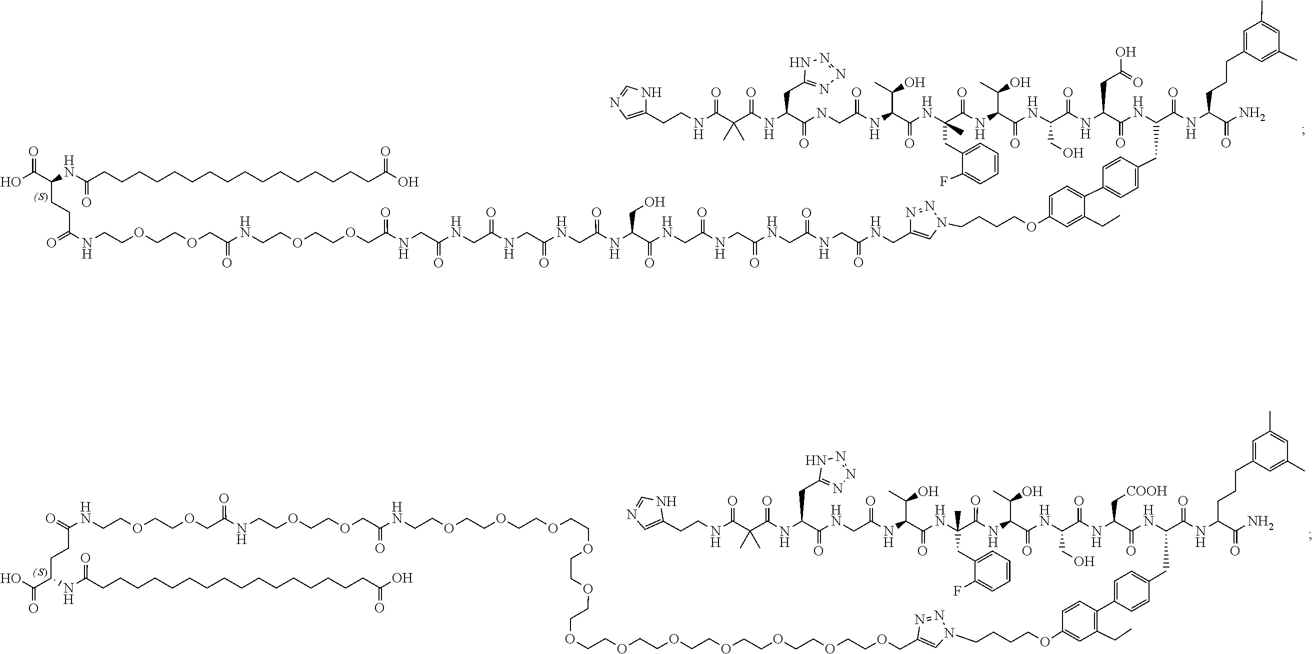

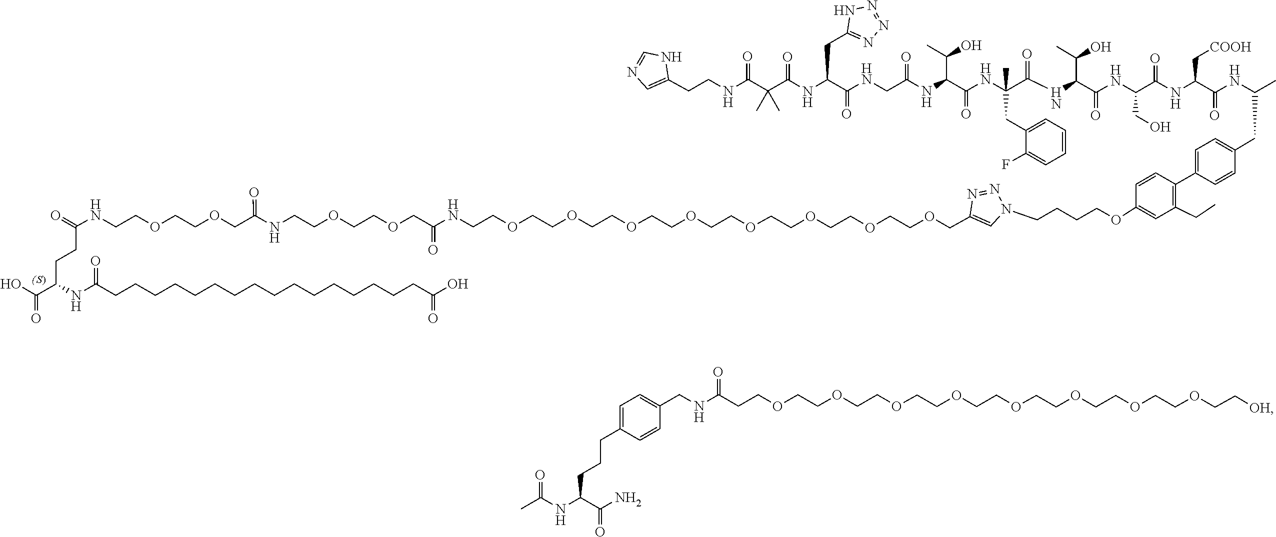

or a pharmaceutically acceptable salt thereof;
optionally, wherein the heavy chain immunoglobulin does not comprise a C-terminal lysine or lysine and glycine.

The present invention includes an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   that is conjugated, optionally through a linker, to a payload having the structure selected from the group consisting of (SEQ ID NOS 465, 576, 466-495, 610, 496-497, 611, 498-505, respectively, in order of appearance):

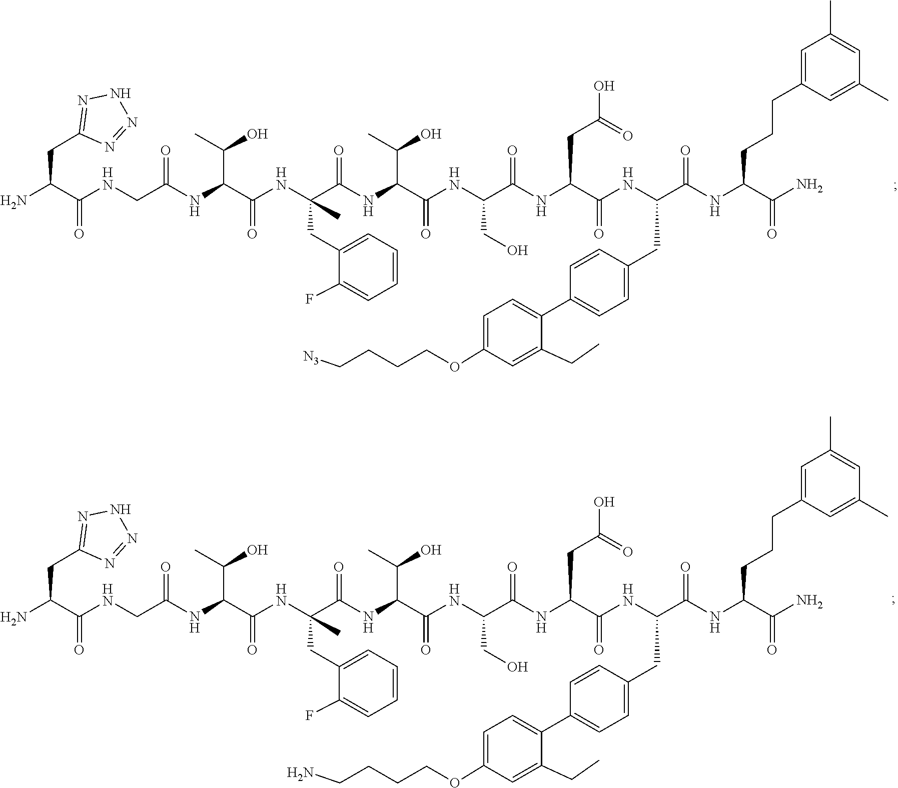 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(N)=O)cc1 ; CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(N)=O)cc1]

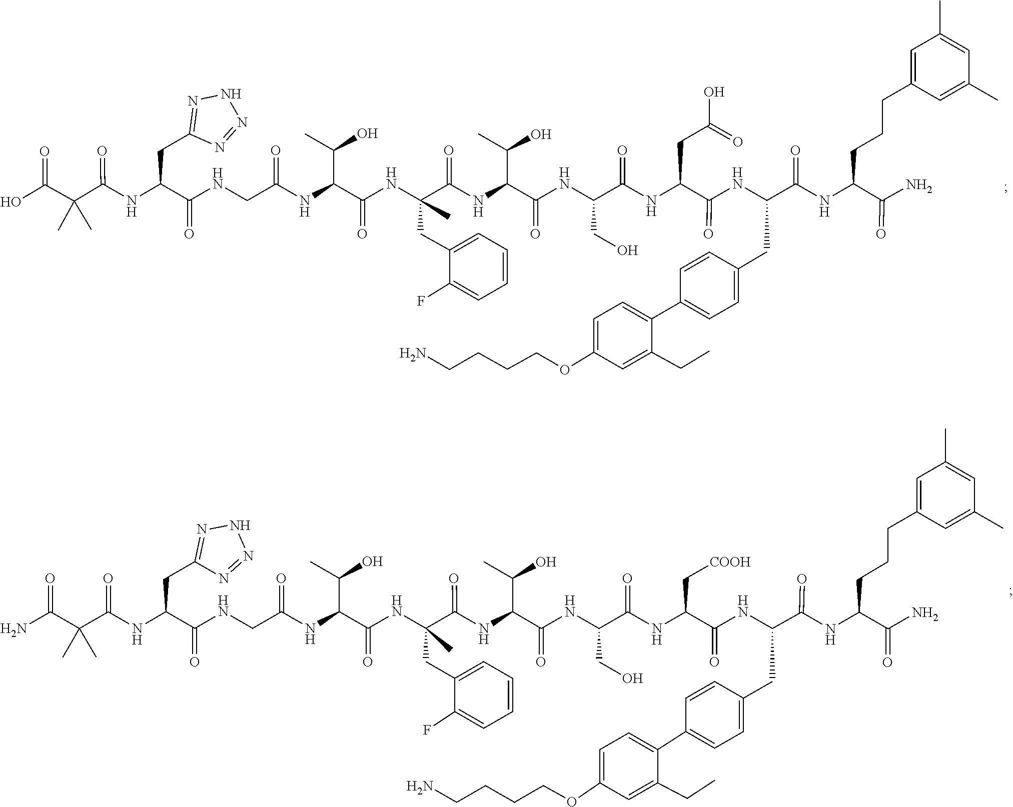 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)O)[C@@H](C)O)[C@@H](C)O)C(=O)N[C@@H](CCCc2cc(C)cc(C)c2)C(N)=O)cc1 ; CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(N)=O)cc1]

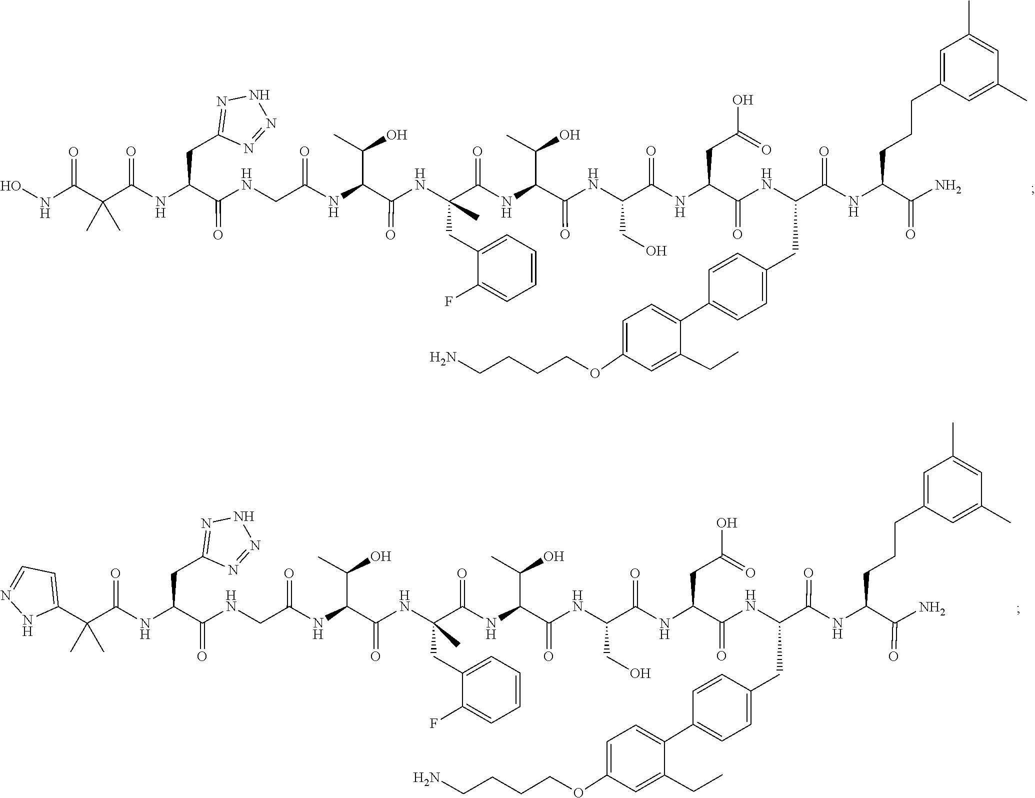 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NO)[C@@H](C)O)[C@@H](C)O)C(=O)N[C@@H](CCCc2cc(C)cc(C)c2)C(N)=O)cc1 ; CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)c2ccn[nH]2)[C@@H](C)O)[C@@H](C)O)C(=O)N[C@@H](CCCc2cc(C)cc(C)c2)C(N)=O)cc1]

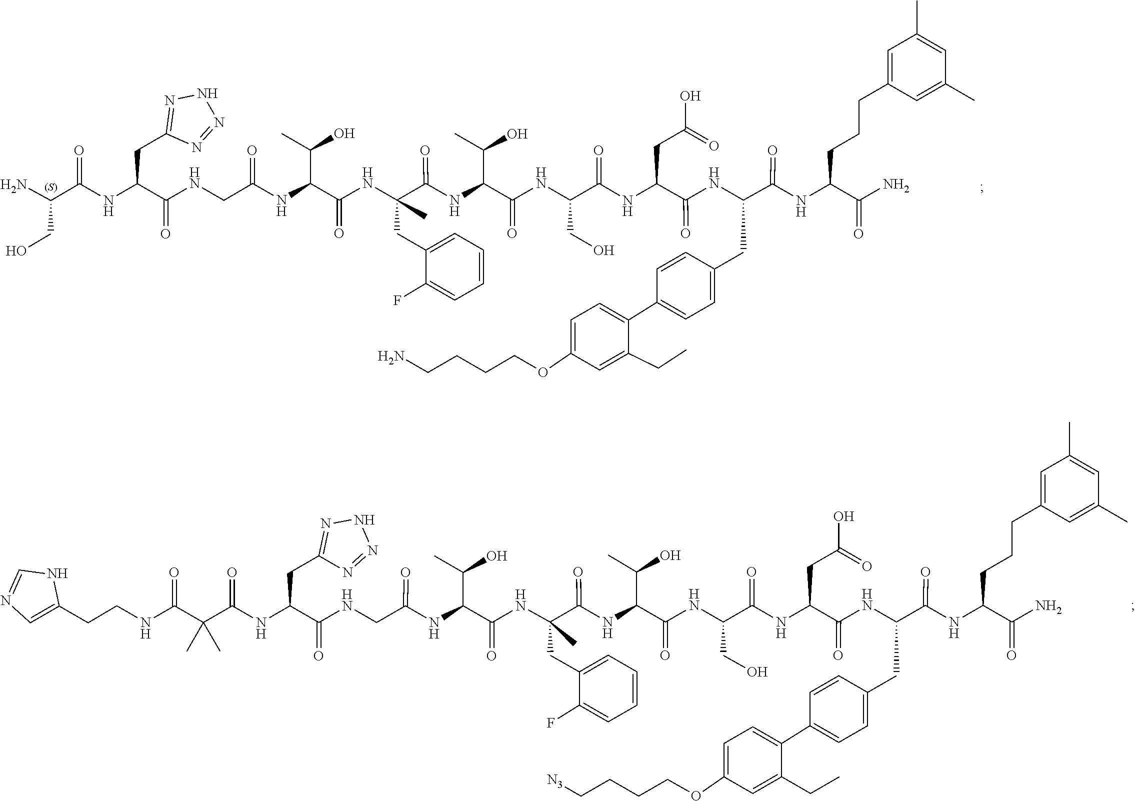

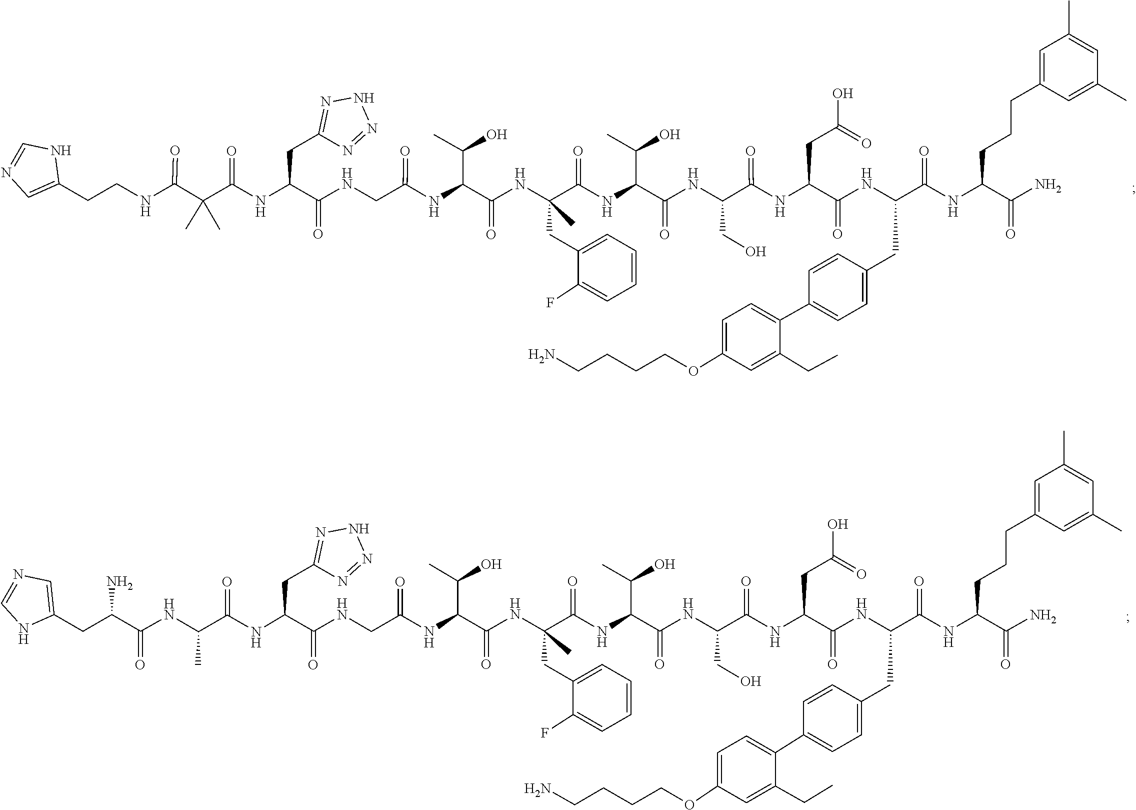 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1 ; CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)[C@H](C)NC(=O)[C@@H](N)Cc2cnc[nH]2)[C@@H](C)O)[C@@H](C)O)C(=O)N[C@@H](CCCc2cc(C)cc(C)c2)C(N)=O)cc1]

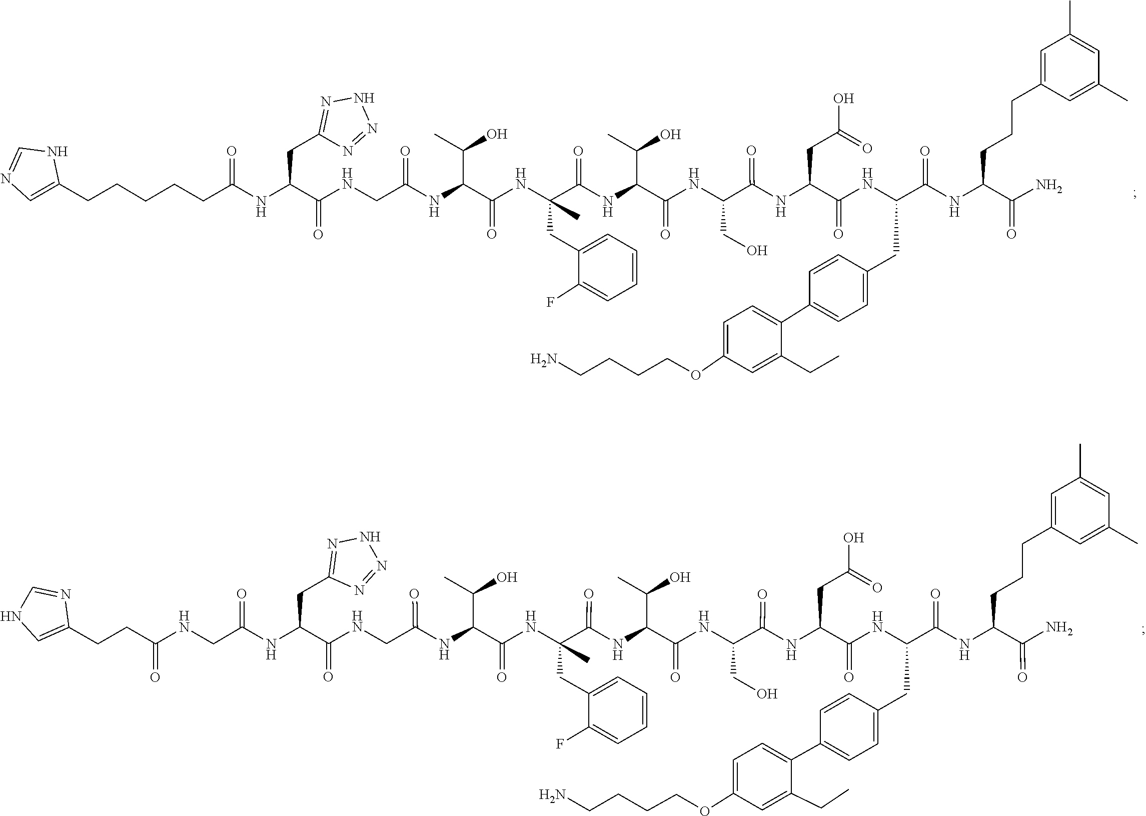 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(N)=O)cc1 ; CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CNC(=O)CCc2c[nH]cn2)[C@@H](C)O)[C@@H](C)O)C(=O)N[C@@H](CCCc2cc(C)cc(C)c2)C(N)=O)cc1]

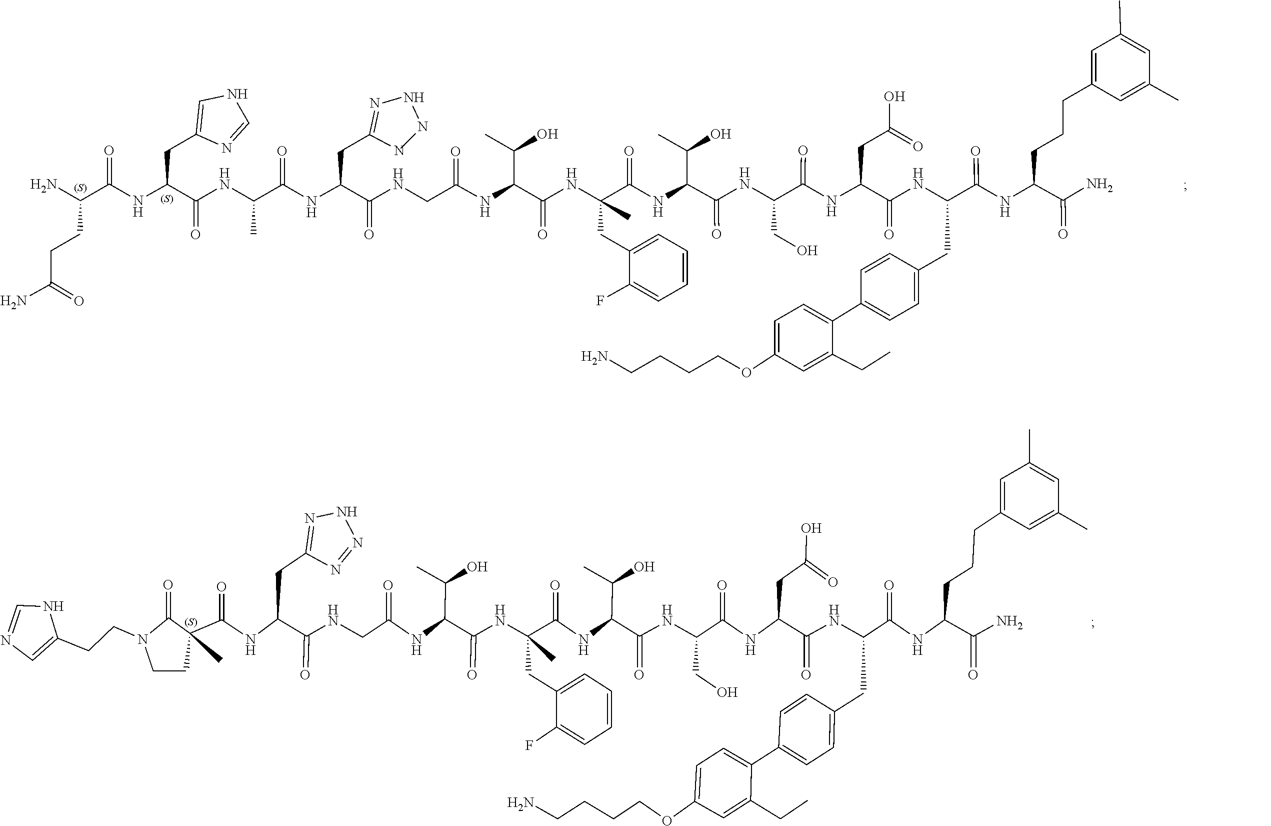

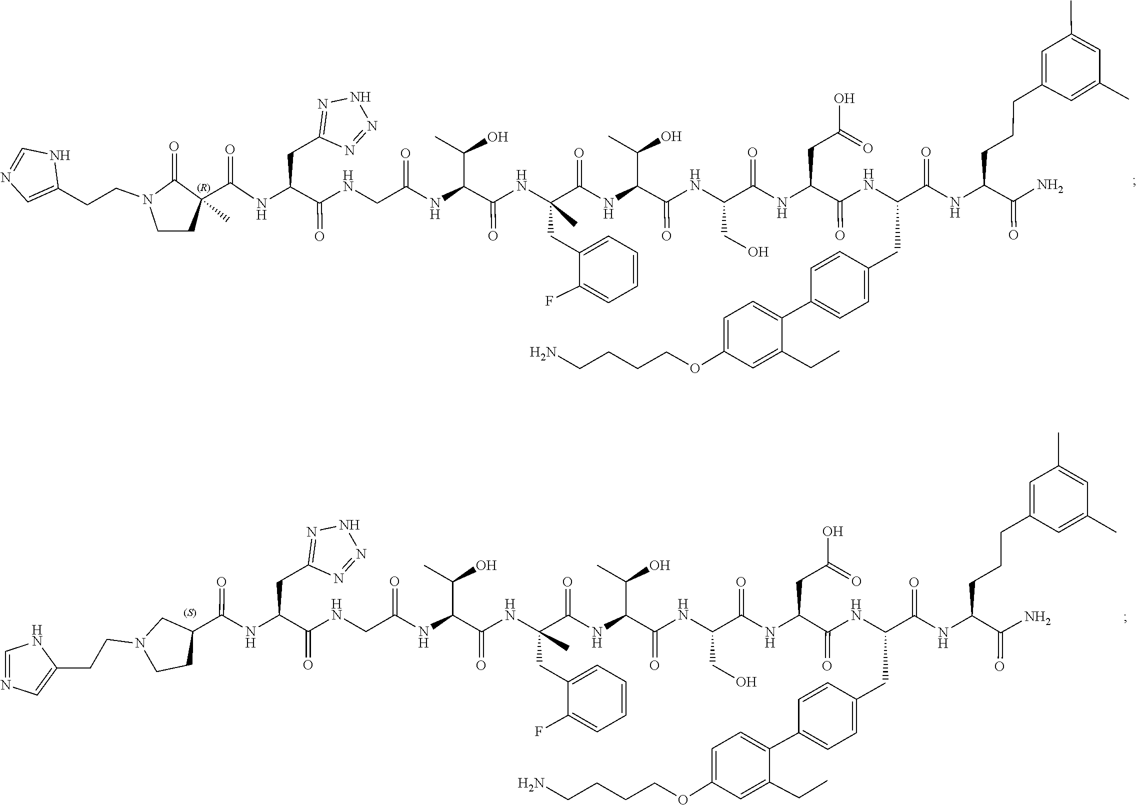

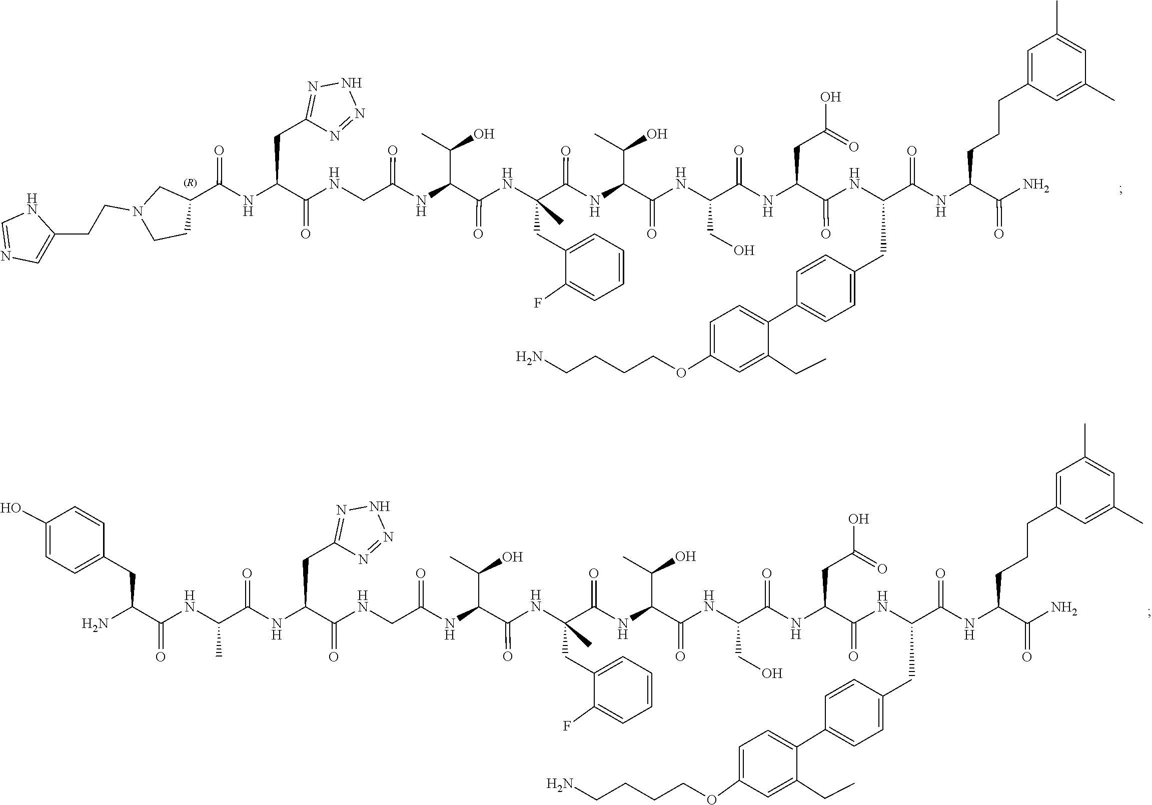

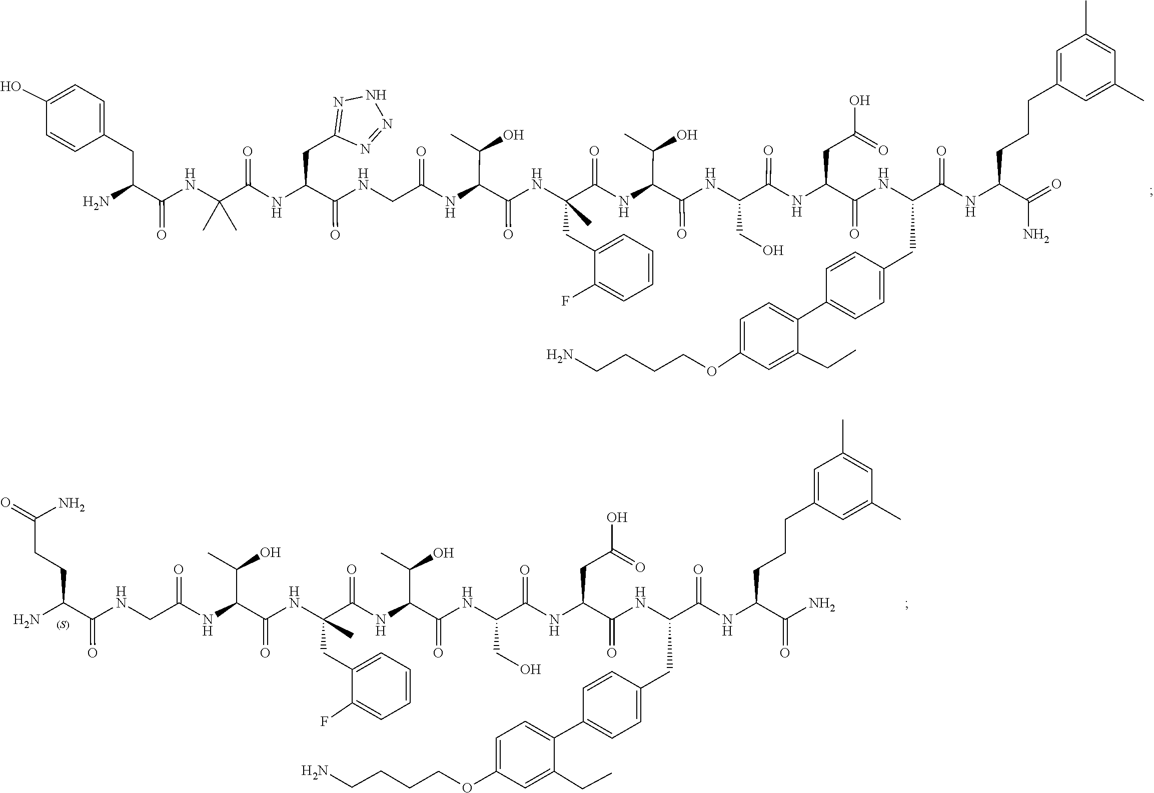

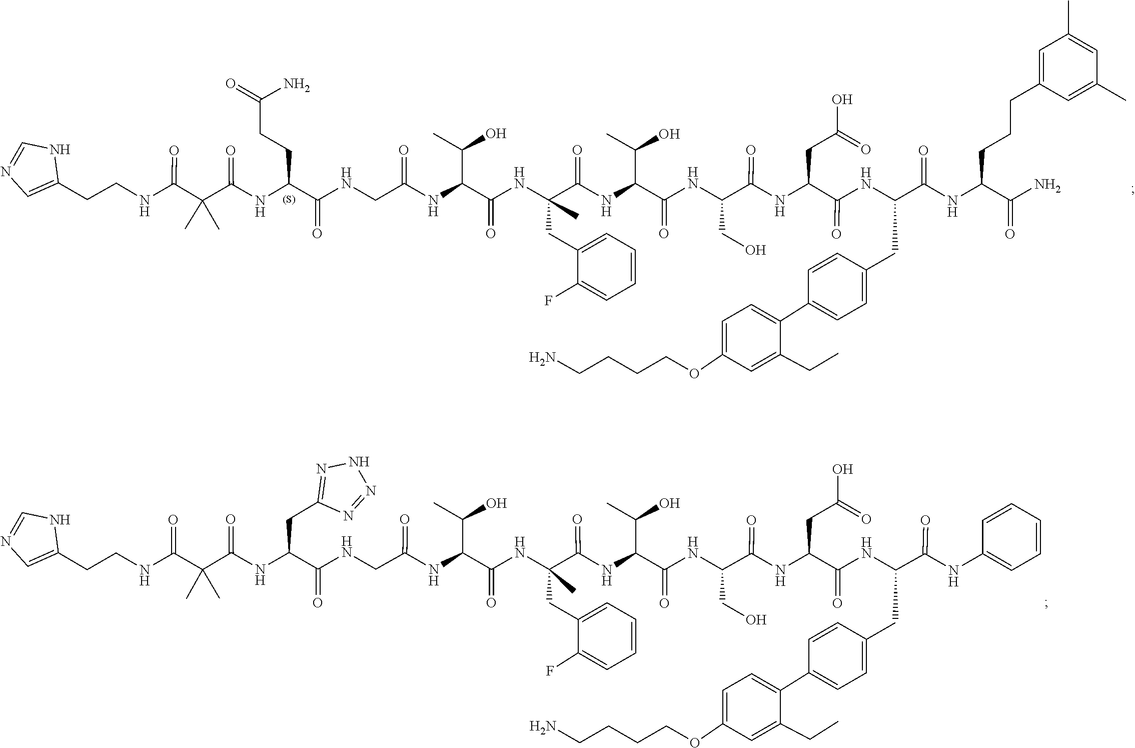

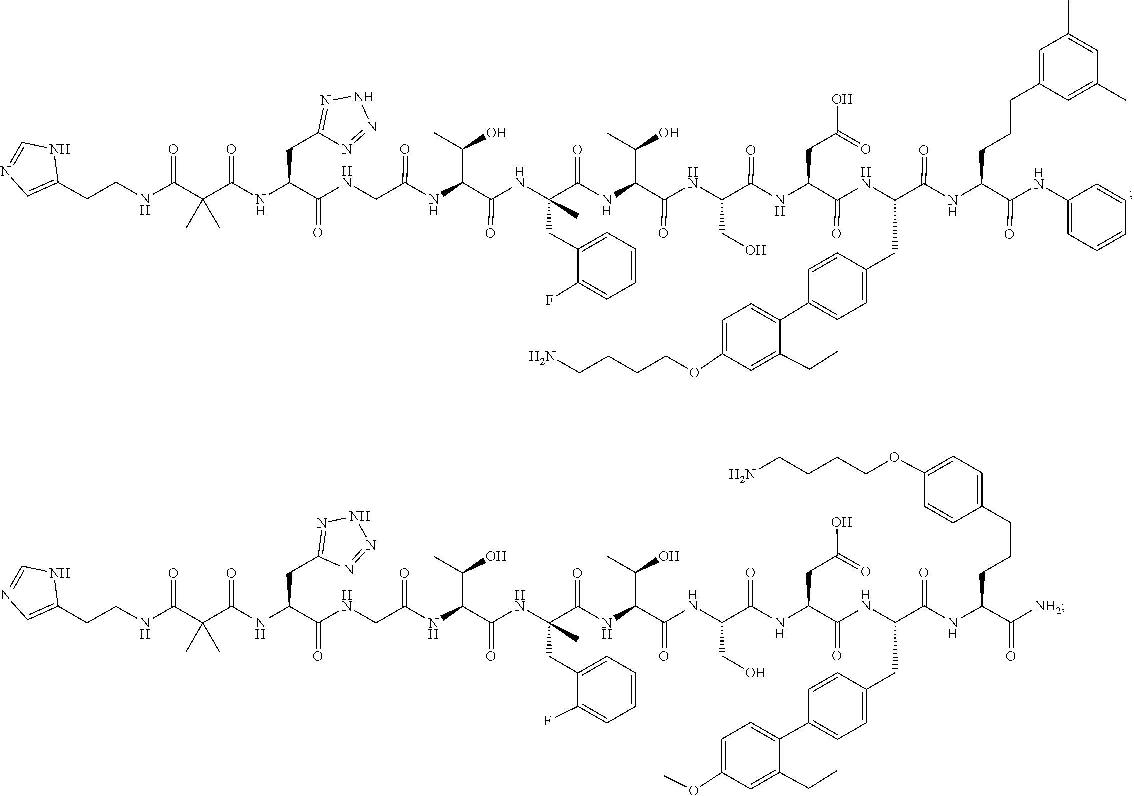 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)Nc2ccccc2)cc1 ; CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCN)cc2)C(N)=O)cc1]

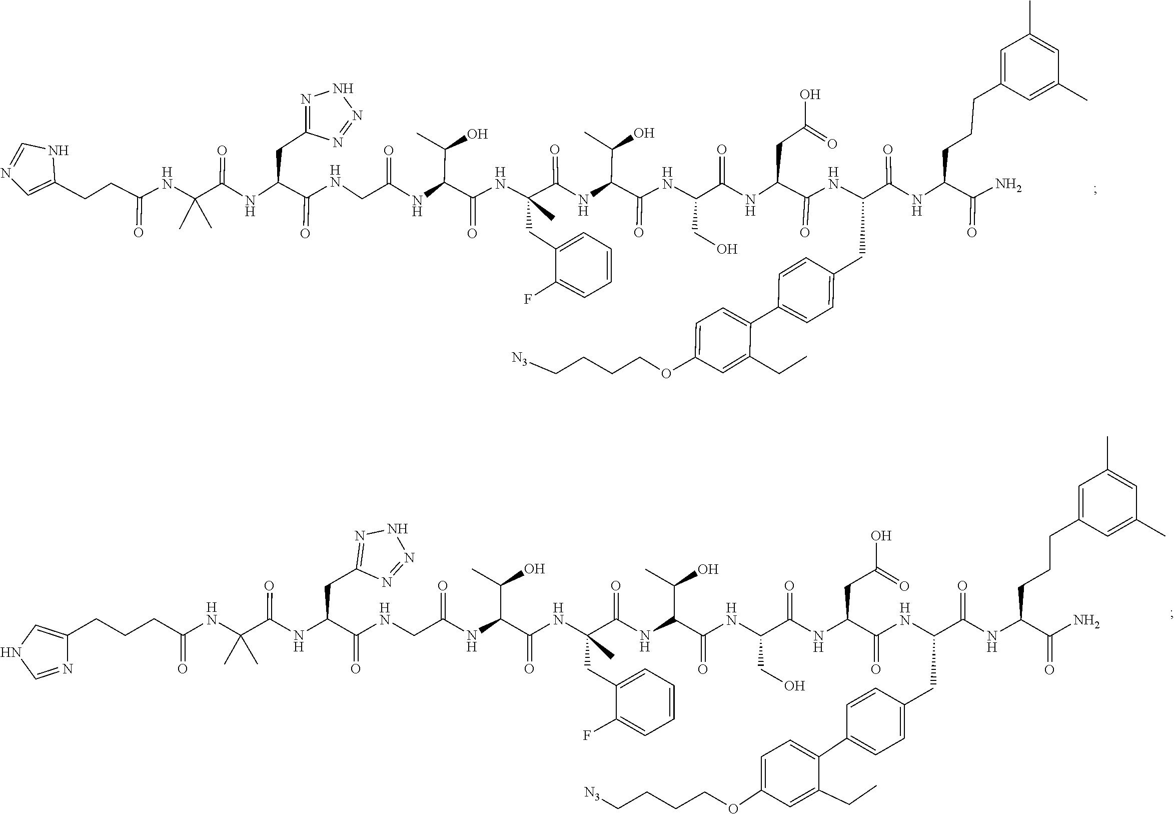

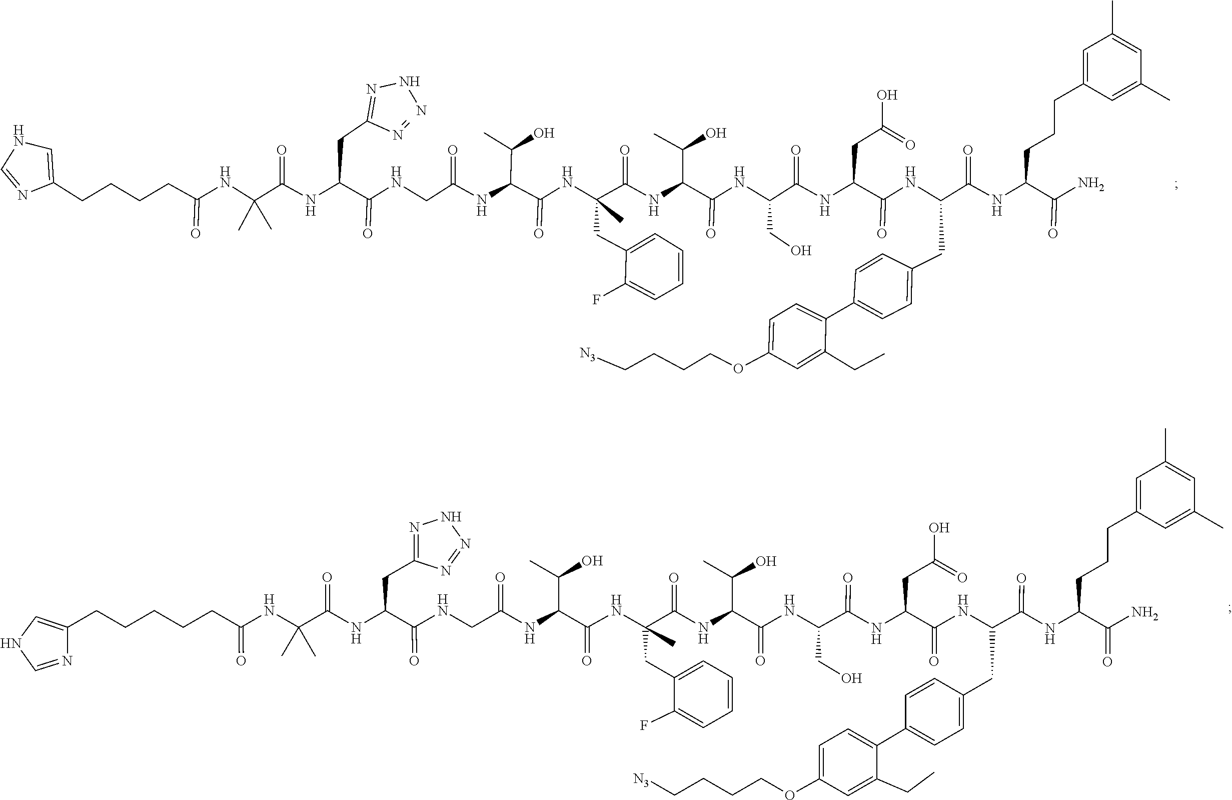

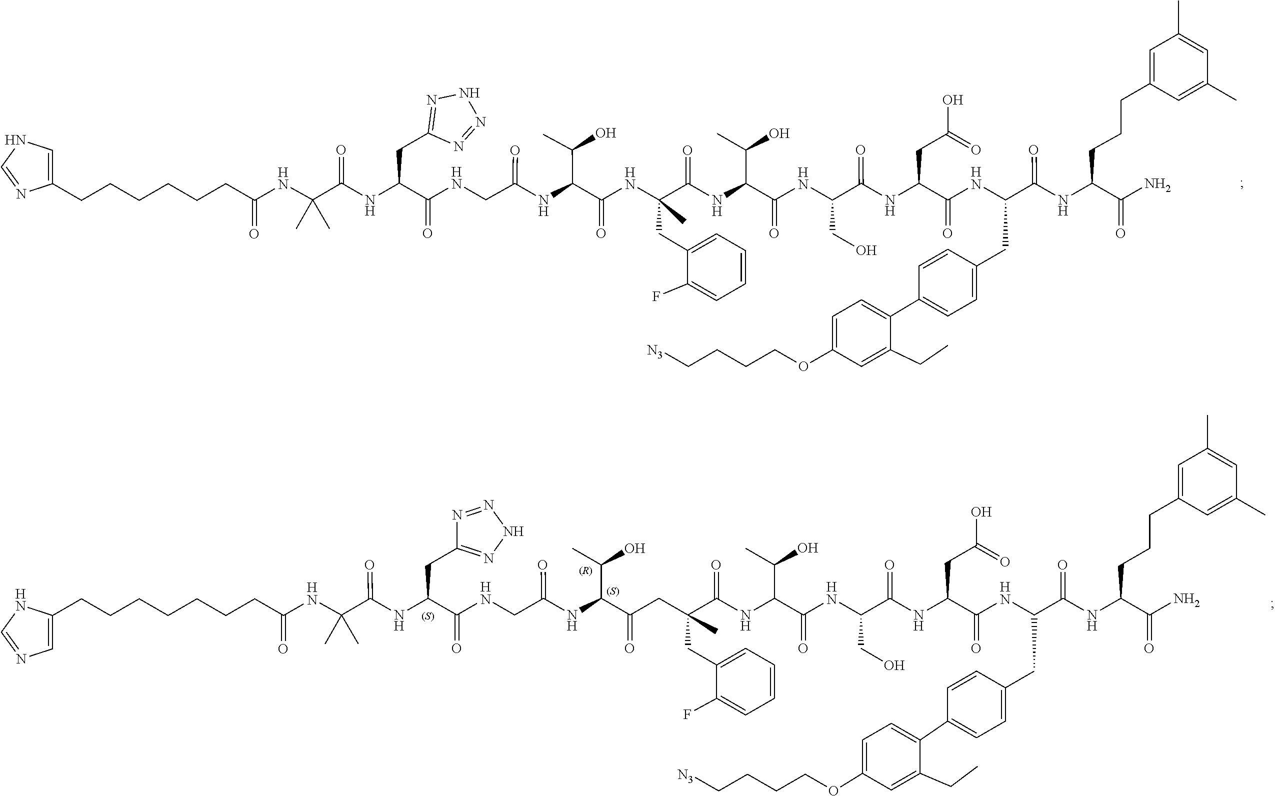

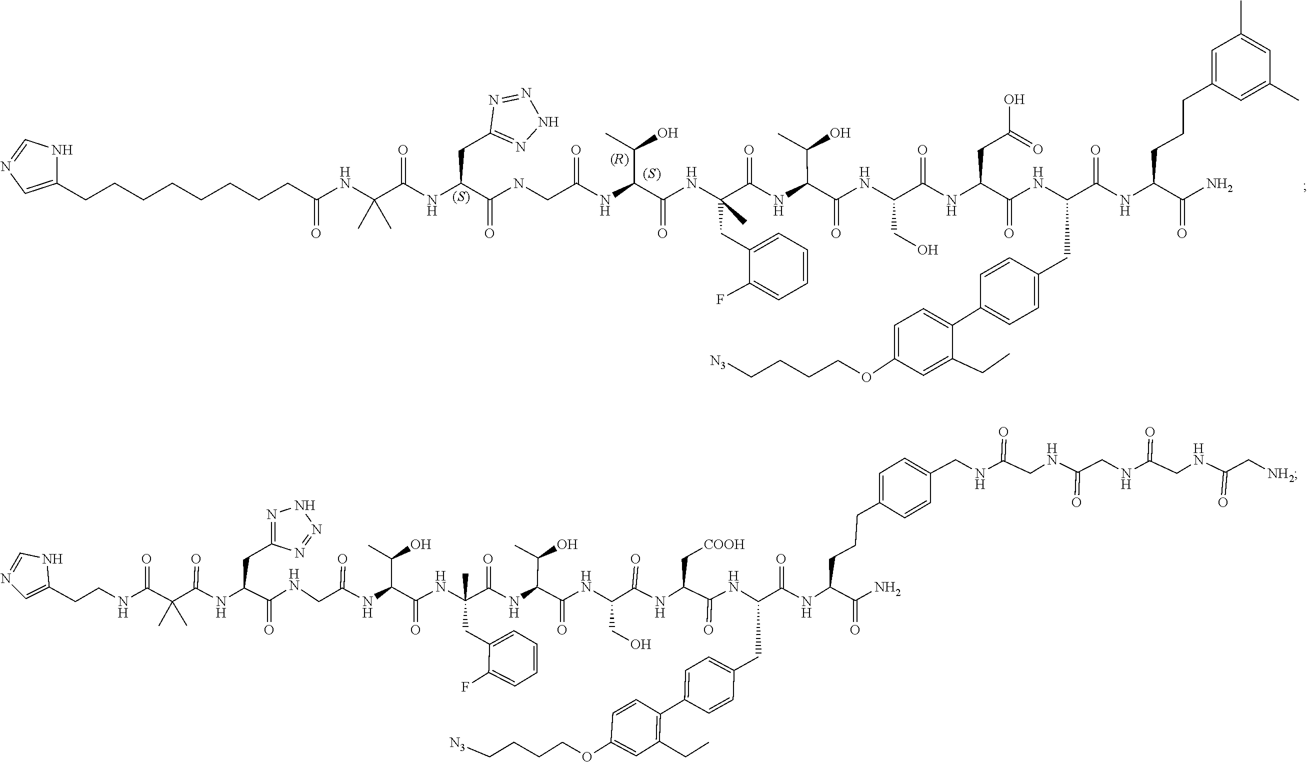

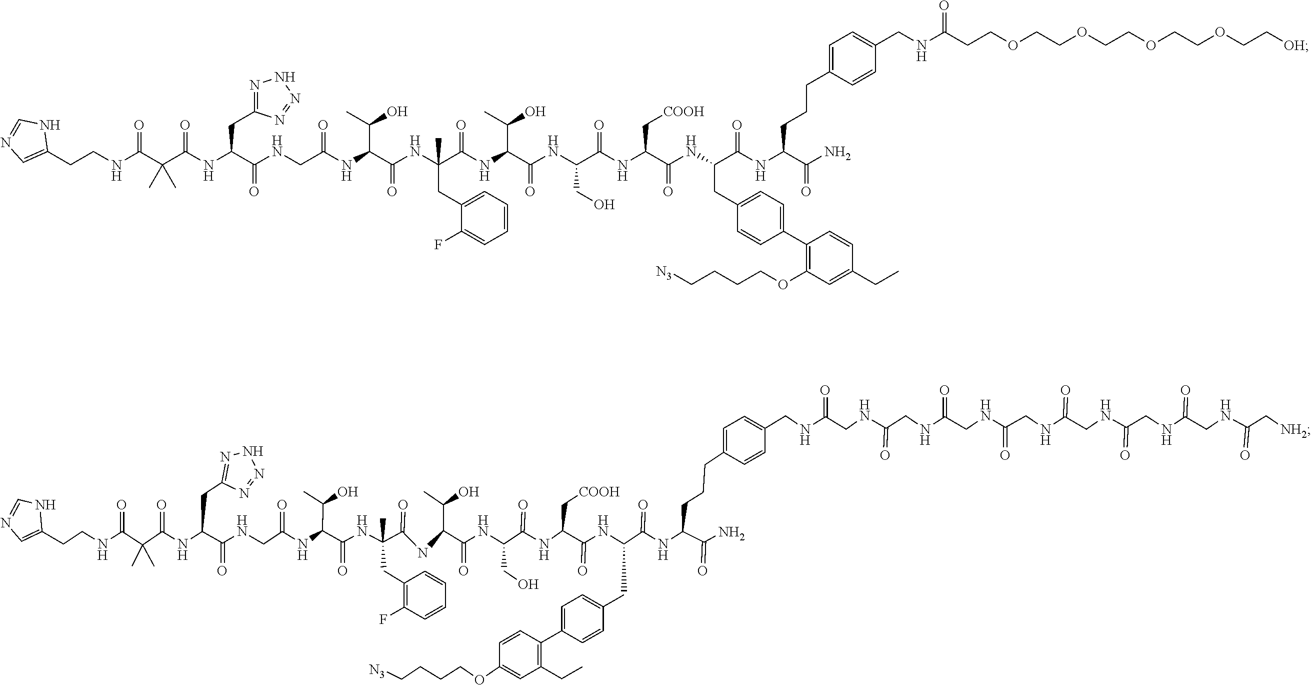

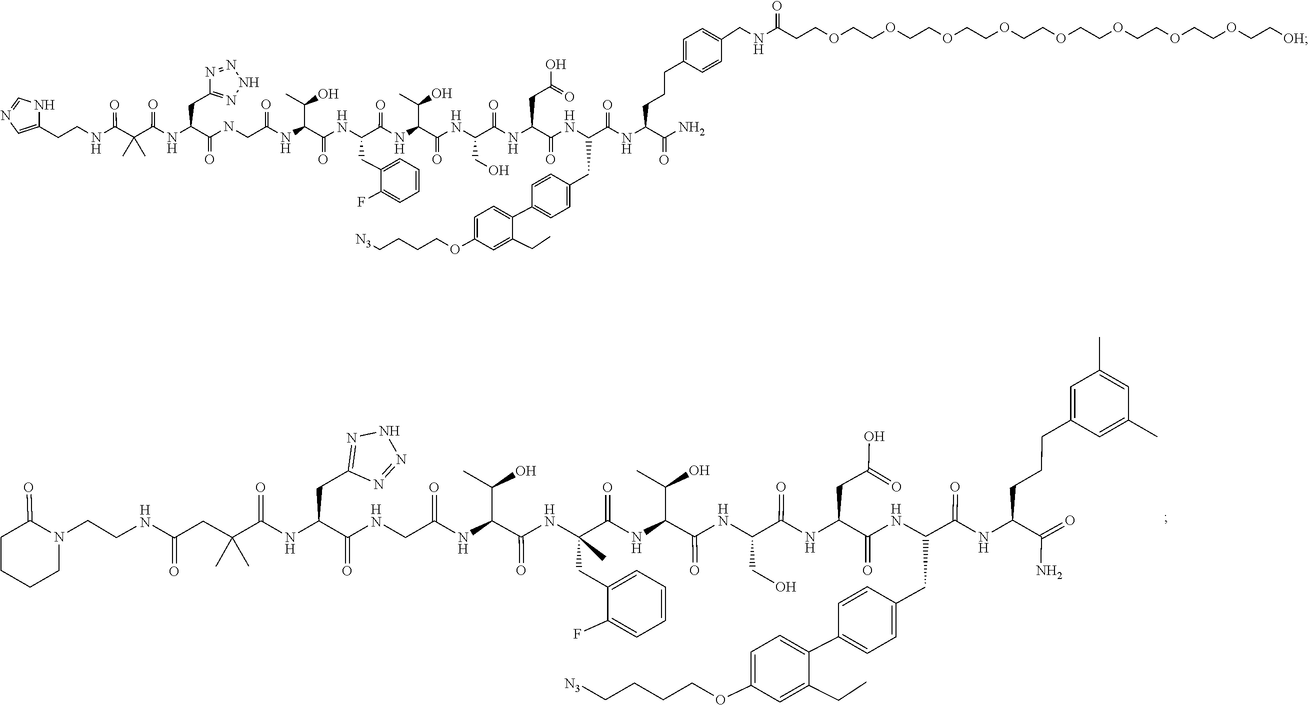

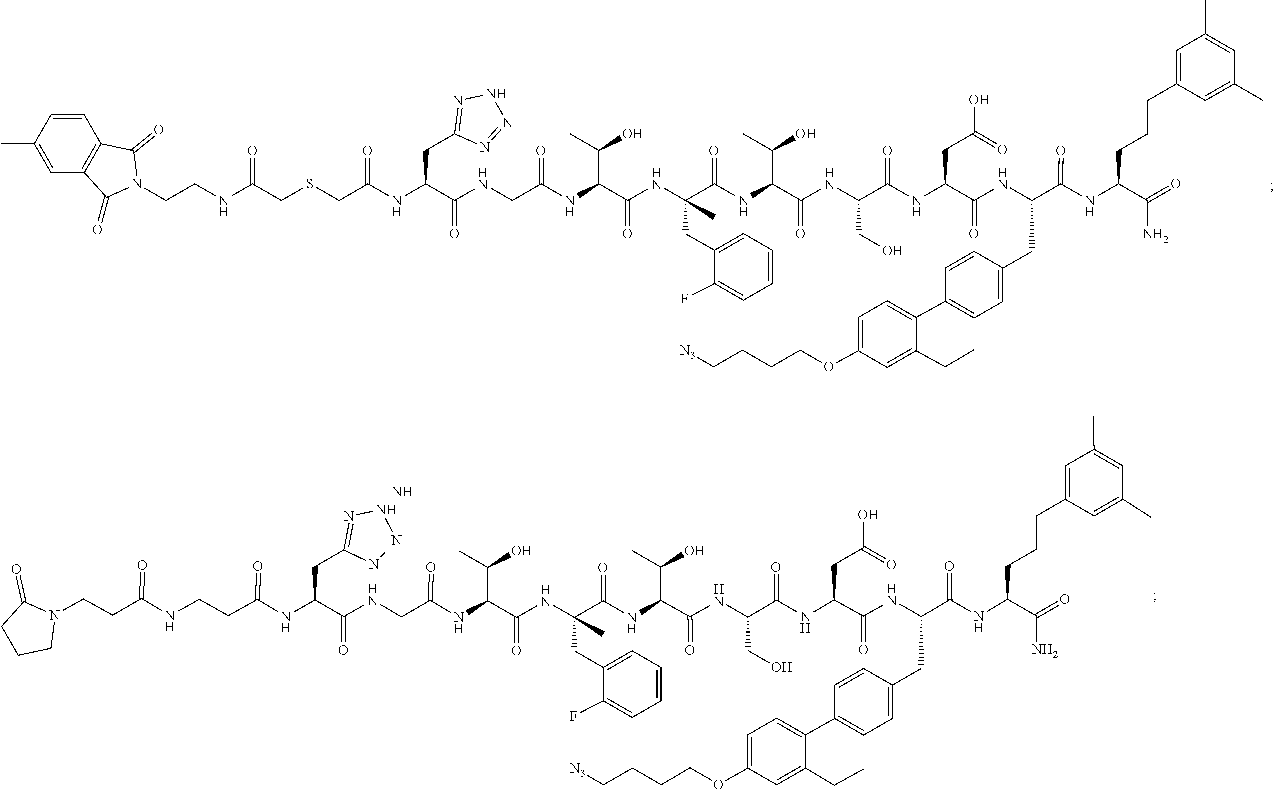

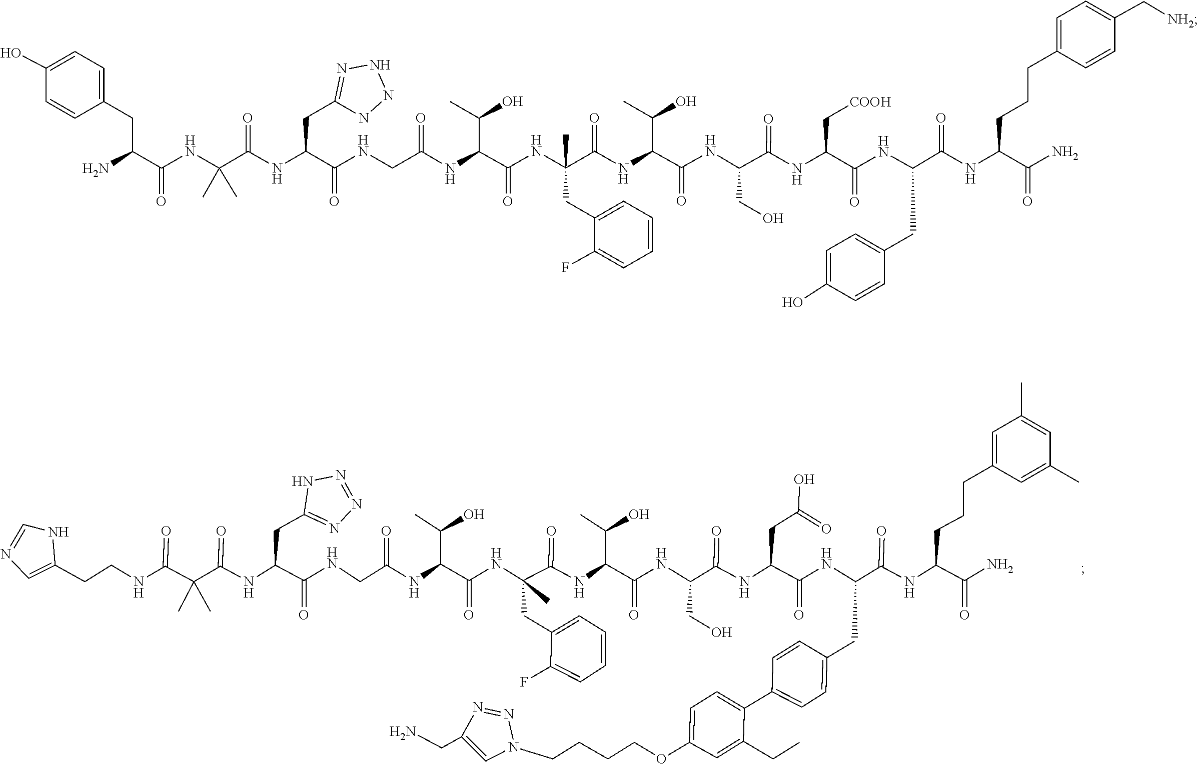

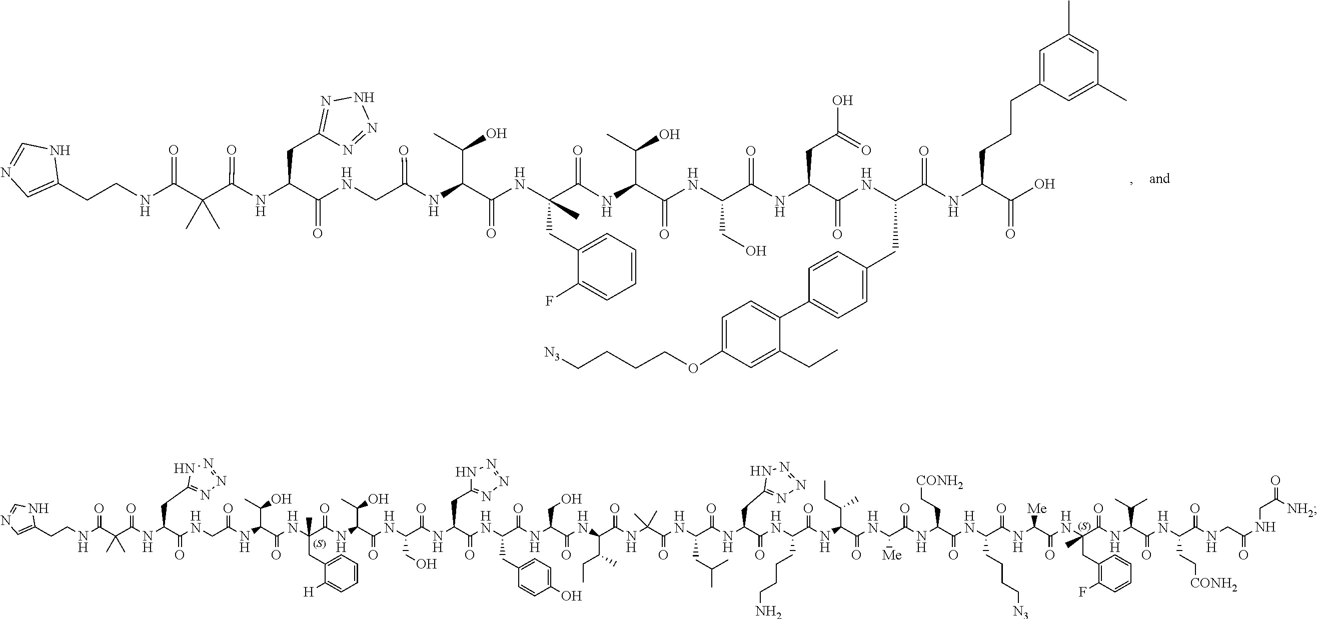

or a pharmaceutically acceptable salt thereof;
optionally, wherein the heavy chain immunoglobulin does not comprise a C-terminal lysine or lysine and glycine.

In yet another aspect, provided herein is an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i);
   which is conjugated to a payload having the structure disclosed as SEQ ID NO: 519:

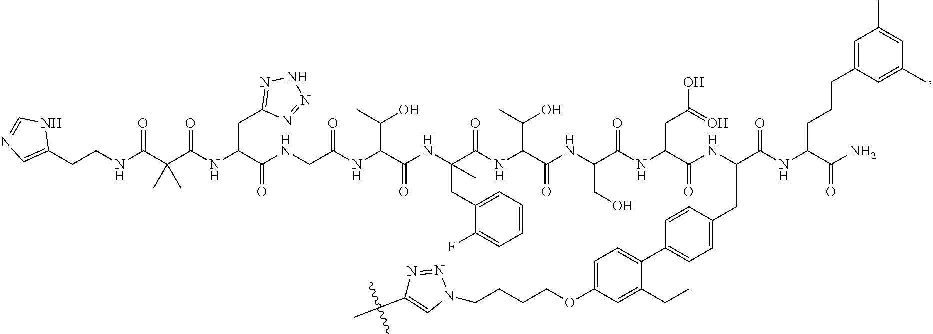 [SMILES: *c1cn(CCCCOc2ccc(-c3ccc(CC(NC(=O)C(CC(=O)O)NC(=O)C(CO)NC(=O)C(NC(=O)C(C)(Cc4ccccc4F)NC(=O)C(NC(=O)CNC(=O)C(Cc4nn[nH]n4)NC(=O)C(C)(C)C(=O)NCCc4cnc[nH]4)C(C)O)C(C)O)C(=O)NC(CCCc4cc(C)cc(C)c4)C(N)=O)cc3)c(CC)c2)nn1]

wherein

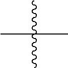

is the point of attachment of the payload to the antibody or the antigen-binding fragment thereof directly or through a linker;
optionally, wherein the heavy chain immunoglobulin does not comprise a C-terminal lysine or lysine and glycine.

In one embodiment, the payload on an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i); has the structure disclosed as SEQ ID NO: 519:

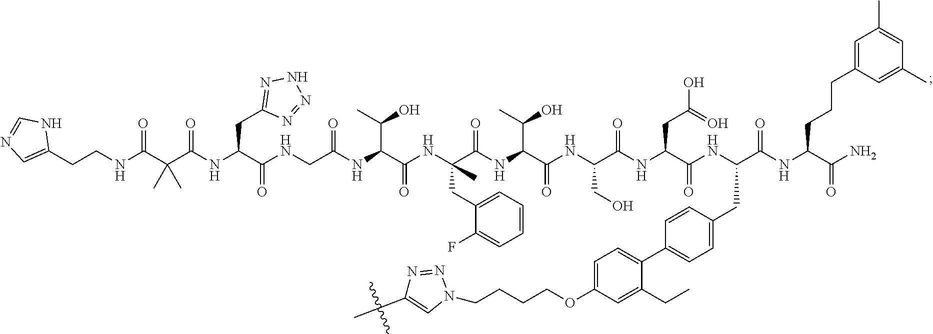 [SMILES: *c1cn(CCCCOc2ccc(-c3ccc(C[C@H](NC(=O)[C@H](CC(=O)O)NC(=O)[C@H](CO)NC(=O)[C@@H](NC(=O)[C@](C)(Cc4ccccc4F)NC(=O)[C@@H](NC(=O)CNC(=O)[C@H](Cc4nn[nH]n4)NC(=O)C(C)(C)C(=O)NCCc4cnc[nH]4)[C@@H](C)O)[C@@H](C)O)C(=O)N[C@@H](CCCc4cc(C)cc(C)c4)C(N)=O)cc3)c(CC)c2)nn1]

optionally, wherein the heavy chain immunoglobulin does not comprise a C-terminal lysine or lysine and glycine.

In yet another aspect, provided herein is an ATDC comprising a Glucagon-like peptide-1 receptor (GLP1R)-targeting antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i); and a linker-payload having the structure disclosed as SEQ ID NO: 507:

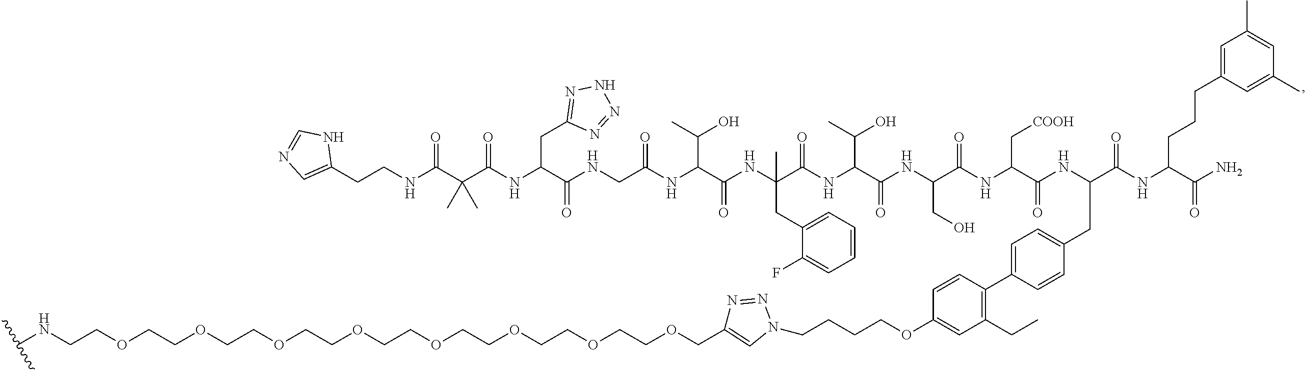 [SMILES: *NCCOCCOCCOCCOCCOCCOCCOCCOCc1cn(CCCCOc2ccc(-c3ccc(CC(NC(=O)C(CC(=O)O)NC(=O)C(CO)NC(=O)C(NC(=O)C(C)(Cc4ccccc4F)NC(=O)C(NC(=O)CNC(=O)C(Cc4nn[nH]n4)NC(=O)C(C)(C)C(=O)NCCc4cnc[nH]4)C(C)O)C(C)O)C(=O)NC(CCCc4cc(C)cc(C)c4)C(N)=O)cc3)c(CC)c2)nn1]

wherein

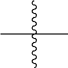

is the point of attachment of the linker-payload to the antibody or the antigen-binding fragment thereof;
optionally, wherein the heavy chain immunoglobulin does not comprise a C-terminal lysine or lysine and glycine.

In an embodiment of the invention, the linker-payload of an ATDC that includes an antibody or an antigen-binding fragment thereof that binds specifically to GLP1R and, e.g., that
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or fragment of (i); and/or
(iii) is an antibody or antigen-binding fragment thereof that binds to the same epitope of GLP1R as said antibody or fragment of (i); has the structure disclosed as SEQ ID NO: 507:

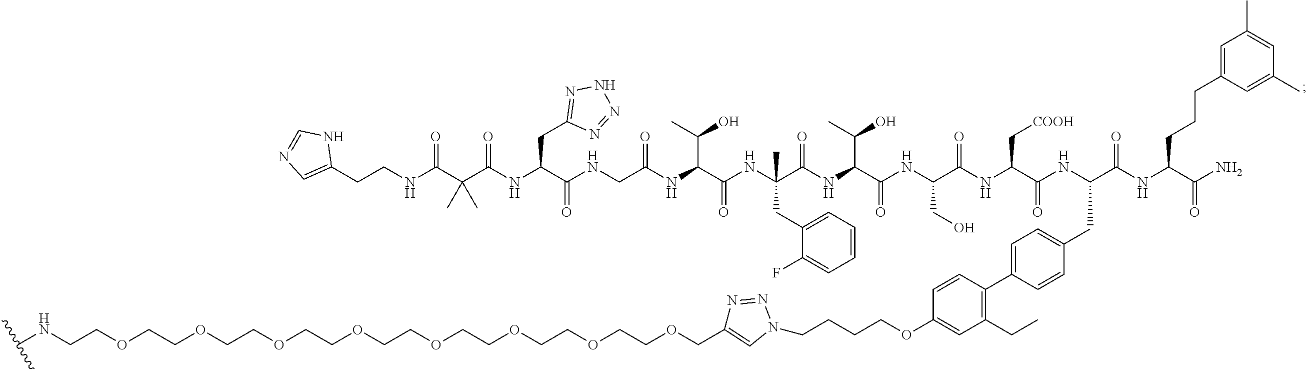

optionally, wherein the heavy chain immunoglobulin does not comprise a C-terminal lysine or lysine and glycine.

These and other aspects of the present disclosure will become apparent to those skilled in the art after a reading of the following detailed description of the disclosure, including the appended claims.

### BRIEF DESCRIPTION OF THE DRAWINGS

FIG. 1A shows a schematic representation of an exemplary antibody-tethered drug conjugate (ATDC) design and its mechanism of action.

FIG. 1B shows a schematic representation of a conventional antibody-drug conjugate (ADC) design and its mechanism of action.

FIG. 2 shows a model of an antibody-tethered drug conjugate having an antibody binding to extracellular domain (ECD) and a payload binding to the transmembrane domain (TMD).

FIG. 3A shows a schematic representation of GLP1 (7-36) amide (SEQ ID NO: 4). The numbers above the sequence correspond to the amino acid positions in the proglucagon propeptide. The arrow between position 8 and position 9 indicates the dipeptidyl peptidase-4 (DPP-IV) cleavage site. The arrows between position 9 and position 10, between position 11 and position 12, between position 15 and position 16, between position 17 and position 18, between position 18 and position 19, between position 27 and position 28, between position 28 and position 29, and between position 31 and position 32 indicate the neutral endopeptidase (NEP) cleavage sites. The dashed arrows between position 30 and position 31 and between 32 and 33 indicate cleavage sites by unknown endoprotease(s). The residues at positions 7, 10, 13, 15, 28, and 29 are amino acids which, when substituted, reduce GLP1R binding and cAMP production. The residues at positions 9, 12, 32, and 36 are amino acids which, when substituted, reduce GLP1R binding.

FIG. 3B shows a structure of GLP1R bound to GLP1 (Protein Data Bank ID: 3IOL). References of this structure may be found in Zhang et al. Nature 2017, Chepurny et al. JBC 2019, De Graaf et al. Pharmacological reviews 2016, and Manandhar and Ahn Journal of Medical Chemistry 2014, each of which is incorporated herein by reference in its entirety.

FIG. 4A shows the sequence and structure of a GLP1 peptidomimetic, Peptide 5 (SEQ ID NO: 5). The numbers above the sequence correspond to the amino acid positions in the proglucagon propeptide.

FIG. 4B shows superimposed structures of GLP1R bound to Peptide 5 (Protein Data Bank ID: 5NX2) and GLP1R bound to GLP1 (Protein Data Bank ID: 3IOL) using the GLP1R in 5NX2 as the template. Reference of the 5NX2 structure can be found in Jazayeri A, et al. Nature volume 546, pages 254-258 (2017), which is incorporated herein by reference in its entirety.

FIG. 5 shows a synthetic scheme for making GLP1 peptidomimetic payloads of the present disclosure. Solid Phase Peptide Synthesis on resin was established which efficiently generated the payloads of the present disclosure with good yields. Additional GLP1R peptidomimetic payloads were generated via systematic R1/R2/R3-modifications.

FIGS. 6A-6D demonstrate that the GLP1R peptidomimetic payloads of the present disclosure showed no activation in related GPCRs bioassays.

FIGS. 7A-7B show that shorter linker GLP1R ATDCs showed greater potency over the control ATDCs.

FIG. 8 shows that the lead linker-payload showed optimal in vitro ADME profile with no in vitro cardiotoxicity and mutagenic potential and its ATDC is highly stable in plasmas.

FIG. 9A shows two methods for conjugating linker-payloads to an antibody of the present disclosure.

FIG. 9B shows a representative hydrophobic interaction chromatography (HIC) graph of anti-GLP1R ATDC drug loading profile. HIC was used in the conjugatability screening to triage ATDCs that show low conjugation yields, low DAR, high aggregates and poor Biacore-binding.

FIG. 10 shows CRE-dependent luciferase reporter activity by anti-GLP1R ATDCs. Anti-GLP1R ATDCs showed better in vitro potency than isotype control ATDCs. Unconjugated mAbs did not activate hGLP1R cells (not shown). ATDCs did not activate Glucagon-like peptide-2 receptor (GLP2R), glucagon receptor (GCGR), or gastric inhibitory polypeptide receptor (GIPR) (not shown).

FIG. 11A shows cyclic AMP response element (CRE)-dependent luciferase reporter activity by anti-GLP1R ATDCs in the presence of unconjugated anti-GLP1R antibodies. It shows that the unconjugated anti-GLP1R mAb concentrations <10 nM had no impact on anti-GLP1R ATDC activity. 100 nM unconjugated anti-GLP1R mAb reduced anti-GLP1R ATDC potency by 3.8-fold. The assay was performed by adding unconjugated anti-GLP1R mAb first, then immediately adding anti-GLP1R ATDC, and incubating for 4 hours.

FIG. 11B shows the data corresponding to the graphs in FIG. 11A.

FIG. 12 shows a schematic representation of an exemplary GLP1R Q-tag mAb-GLP1R agonist conjugate of the present disclosure.

FIG. 13 shows a general synthetic scheme for preparing GLP1 peptidomimetics according to the disclosure.

FIG. 14 shows a sequence for solid-supported synthesis of GLP1 peptidomimetic payloads P1 and P8 according to the disclosure.

FIG. 15 shows a sequence for solid-supported synthesis of GLP1 peptidomimetic payloads P2 and P9 according to the disclosure.

FIG. 16 shows a sequence for solid-supported synthesis of GLP1 peptidomimetic payloads P3, P4, P5, P6, P7, P11, P13, P14, P15, P16 and P17 according to the disclosure.

FIGS. 17A and 17B show a sequence for solid-supported synthesis of GLP1 peptidomimetic payloads P10, P12, P18, P19, P25, P26, P27, P28, P29, P30, P31, P36, P37, and P38 according to the disclosure.

FIG. 18 shows a sequence for solid-supported synthesis of GLP1 peptidomimetic payloads P20 and P21 according to the disclosure.

FIG. 19 shows a sequence for solid-supported synthesis of GLP1 peptidomimetic payloads P22 and P23 according to the disclosure.

FIG. 20 shows a sequence for solid-supported synthesis of GLP1 peptidomimetic payload P24 according to the disclosure.

FIG. 21 shows a sequence for solid-supported synthesis of GLP1 peptidomimetic payloads P32, P33, P34 and P35 according to the disclosure.

FIG. 22 shows a sequence for solid-supported synthesis of GLP1 peptidomimetic payload P39 according to the disclosure.

FIG. 23 shows a sequence for solid-supported synthesis of GLP1 peptidomimetic payload P40 according to the disclosure.

FIG. 24 shows a sequence for solid-supported synthesis of GLP1 peptidomimetic payload P41 according to the disclosure.

FIG. 25 shows a sequence for solid-supported synthesis of GLP1 peptidomimetic payload P42 according to the disclosure.

FIG. 26 shows a synthetic route for preparation of Linker-Payloads LP1, LP2, LP3, LP4 and LP5 according to the disclosure.

FIG. 27 shows a synthetic route for preparation of Linker-Payloads LP6 and LP7 according to the disclosure.

FIG. 28 shows a synthetic route for preparation of Linker-Payloads LP8, LP9, LP10 and LP11 according to the disclosure.

FIG. 29 shows a synthetic route for preparation of Linker-Payload LP12 according to the disclosure.

FIG. 30 shows a synthetic route for preparation of Linker-Payloads LP13 and LP14 according to the disclosure.

FIG. 31 shows a synthetic route for preparation of Linker-Payloads LP15 and LP18 according to the disclosure.

FIG. 32 shows a synthetic route for preparation of Linker-Payload LP17 according to the disclosure.

FIG. 33 shows a synthetic route for preparation of Linker-Payloads LP18 and LP20 according to the disclosure.

FIG. 34 shows a synthetic route for preparation of Linker-Payload LP19 according to the disclosure.

FIG. 35 shows a synthetic route for preparation of Linker-Payload LP21 according to the disclosure.

FIG. 36 shows a synthetic route for preparation of Linker-Payload LP22 according to the disclosure.

FIG. 37 shows a synthetic route for preparation of Linker-Payload LP23 according to the disclosure.

FIG. 38 shows a synthetic route for preparation of Linker-Payload LP24 according to the disclosure.

FIG. 39 shows a synthetic route for preparation of Linker-Payload LP25 according to the disclosure.

FIG. 40 shows a synthetic route for preparation of Linker-Payload LP26 according to the disclosure.

FIG. 41 shows a synthetic route for preparation of Linker-Payloads LP27 and LP28 according to the disclosure.

FIG. 42 shows a synthetic route for preparation of Linker-Payload LP29 according to the disclosure.

FIG. 43 shows a synthetic route for preparation of Linker-Payload LP30 according to the disclosure.

FIG. 44 shows a synthetic route for preparation of Linker-Payload LP31 according to the disclosure.

FIG. 45 shows a synthetic route for preparation of Linker-Payload LP32 according to the disclosure.

FIG. 46 shows a synthetic route for preparation of Linker-Payload LP33 according to the disclosure.

FIG. 47 shows a synthetic route for preparation of Linker-Payload LP34 according to the disclosure.

FIG. 48 shows a synthetic route for preparation of Linker-Payload LP35 according to the disclosure.

FIG. 49 shows a synthetic route for preparation of Linker-Payloads LP36, LP37, LP38, LP39, LP40, and LP41 according to the disclosure.

FIG. 50 shows a synthetic route for preparation of Linker-Payload LP42 according to the disclosure.

FIG. 51 shows a synthetic route for preparation of Linker-Payload LP43 according to the disclosure.

FIG. 52 shows a synthetic route for preparation of Linker-Payload LP44 according to the disclosure.

FIG. 53 shows a synthetic route for preparation of Linker-Payload LP45 according to the disclosure.

FIG. 54 shows a schematic of a general two-step conjugation procedure for the preparation of site-specific antibody-drug conjugates.

FIG. 55 shows a schematic of a general one-step conjugation procedure for the preparation of site-specific antibody-drug conjugates.

FIG. 56 shows the commander voltage protocol for electrophysiological study. From a holding potential of −80 mV, the voltage was first stepped to −50 mV for 80 ms for leak subtraction, and then stepped to +20 mV for 4800 ms to open hERG channels. After that, the voltage was stepped back down to −50 mV for 5000 ms, causing a “rebound” or tail current, which was measured and collected for data analysis. Finally, the voltage was stepped back to the holding potential (−80 mV, 1000 ms). Voltage command protocol was repeated every 20 sec and performed continuously during the test (vehicle control and test compound).

FIG. 57 shows in vitro stability of anti-GLP1R mAB2-LP11 over a 7-day, 37° C. incubation in mouse, monkey and human plasma.

FIG. 58 shows the effects of GLP1R ATDCs on percent body weight changes in obese GLP1R humanized mice.

FIG. 59 shows the effects of GLP1R ATDCs on blood glucose levels in obese GLP1R humanized mice.

FIG. 60 is a schematic representation of a GLP1R ATDC according to an exemplary embodiment of the disclosure. Such ATDCs form part of the present invention, including those wherein the antibody is REGN15869, REGN18121 or REGN18123.

FIG. 61 is a diagram of an anti-GLP1R antibody appended, via the glutamine residues (Q) in Qtags (LLQGSG (SEQ ID NO: 18)) on each LCVR, with a linker payload (LP) which is M3190. The bond between the glutamine side chain and the linker is shown; and the bond between the N-terminal LCVR residue and the C-terminal Qtag glycine is shown.]=point of attachment of antibody Qtag Gln to linker of M3190. Such ATDCs form part of the present invention, including those wherein the antibody is REGN15869, REGN18121 or REGN18123.

FIG. 62 is a comparison between GLP1 (top) and an ATDC comprising an anti-GLP1R antibody, having a LLQGSG (SEQ ID NO: 18) Qtag, conjugated to a linker-payload which is M3190 (bottom). E* is (S)-2-amino-3-(2H-tetrazol-5-yl)propanoic acid; F* is (S)-2-amino-3-(2-fluorophenyl)-2-methylpropanoic acid; Cap-Aib is 3-((2-(1H-imidazol-5-yl)ethyl)amino)-2,2-dimethyl-3-oxopropanoic acid; AA2 is (S)-2-amino-3-(4′-(4-(4-(25-amino-2,5,8,11,14,17,20,23-octaoxapentacosyl)-1H-1,2,3-triazol-1-yl)butoxy)-2′-ethyl-[1,1′-biphenyl]-4-yl)propanoic acid [AA2 includes linker]; and AA1=(S)-2-amino-5-(3,5-dimethylphenyl)pentanamide. Such ATDCs form part of the present invention, including those wherein the antibody is REGN15869, REGN18121 or REGN18123.

FIG. 63 shows CryoEM reconstructions and epitope of GLP-1R/REGN9268/M3190 complexes. (A) shows CryoEM reconstruction of REGN9268-M3190 Fab bound to GLP-1R/Gs (‘tethered’ complex). (B) shows CryoEM reconstruction of REGN9268 Fab bound to GLP1R/Gs/M3190 (‘untethered’ complex). Density for G proteins is not present in (B) because the map was calculated from a local refinement conducted after signal subtraction of the G proteins. Locations of GLP-1R domains and complex components are labeled in (A), (B), and (C), © showns an expanded view of REGN9268/GLP-1R interface. GLP-1R contact residues (within 4 Å of REGN9268) are shown as stick and labeled.

FIG. 64 shows CryoEM reconstructions and epitope of GLP-1R/REGN15869/M3190 complexes. (A) shows CryoEM reconstruction of REGN15869-M3190 Fab bound to GLP-1R/Gs (‘tethered’ complex). (B) shows CryoEM reconstruction of REGN15869 Fab bound to GLP1R/Gs/M3190 (‘untethered’ complex). Locations of GLP-1R domains and complex components are labeled in (A), (B), and (C), (C) shows an Expanded view of REGN15869/GLP-1R interface. GLP-1R contact residues (within 4 Å of REGN15869) are shown as stick and labeled.

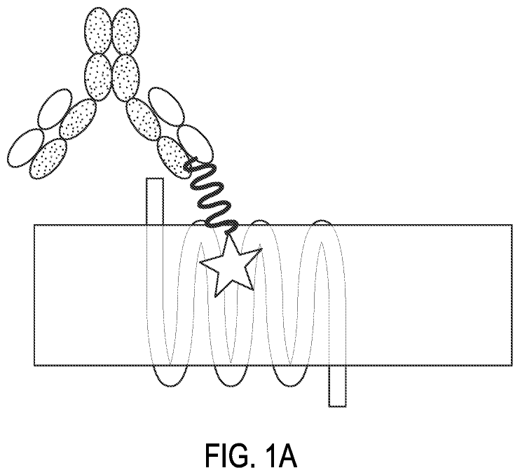

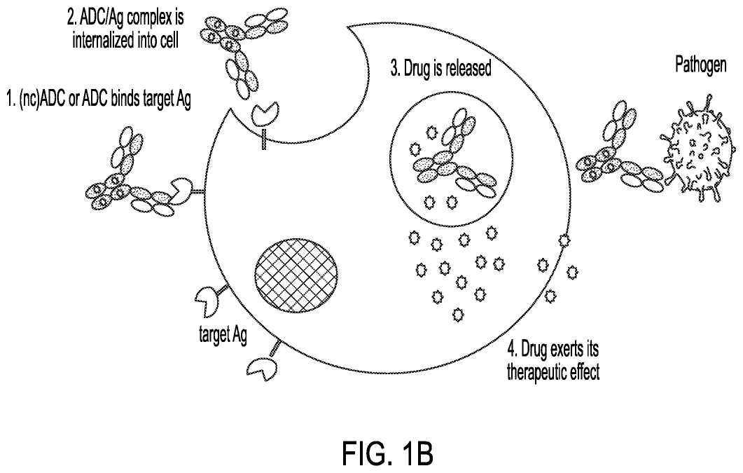

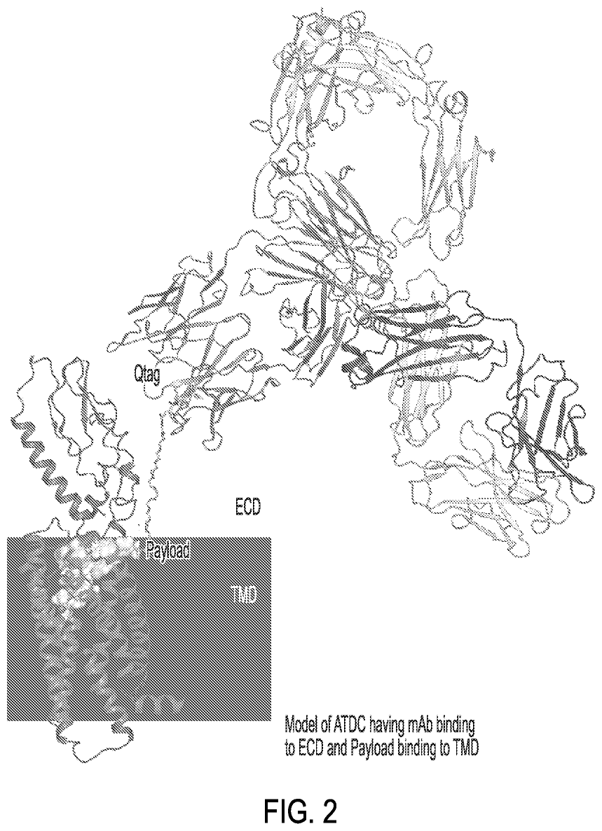

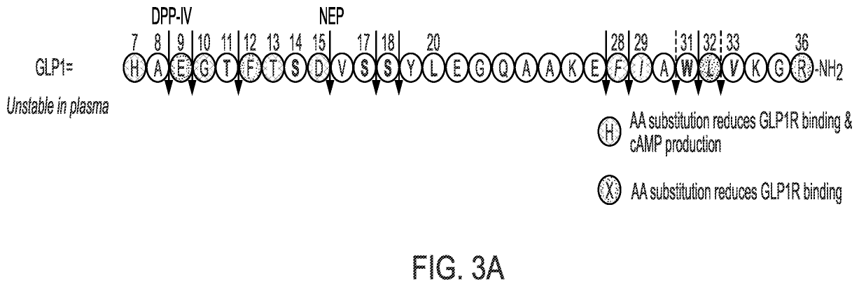

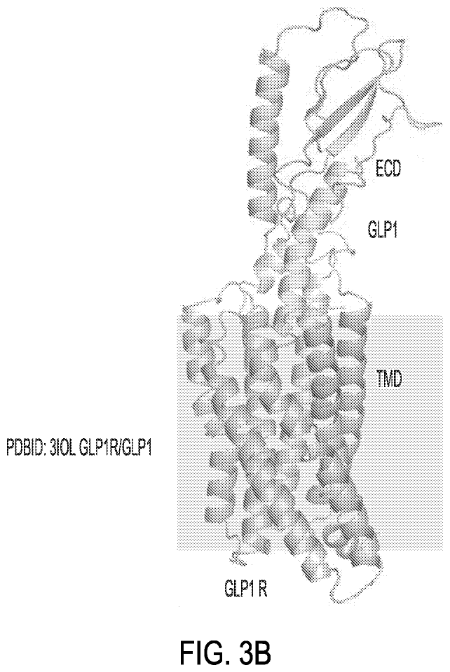

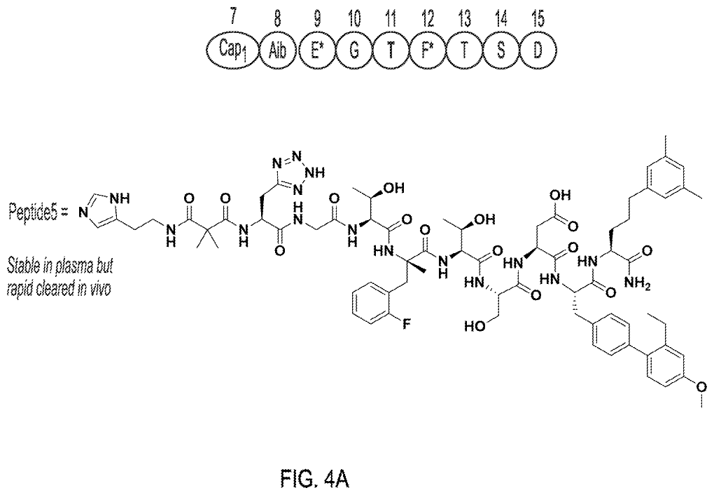

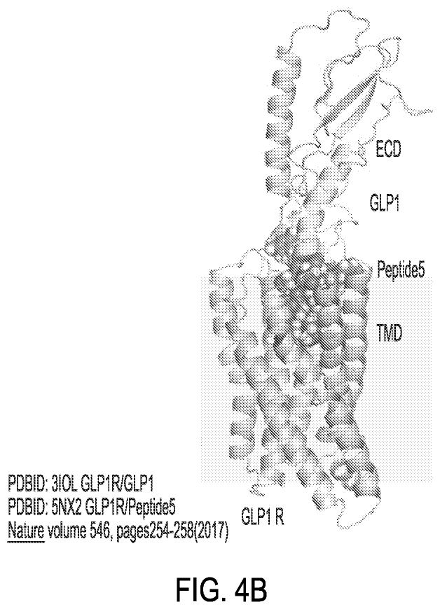

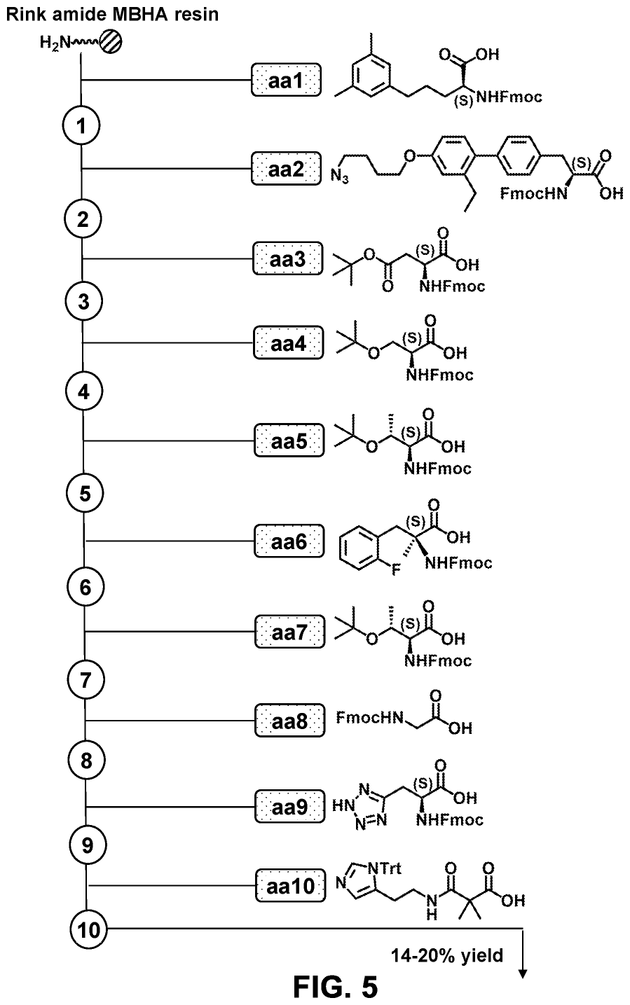

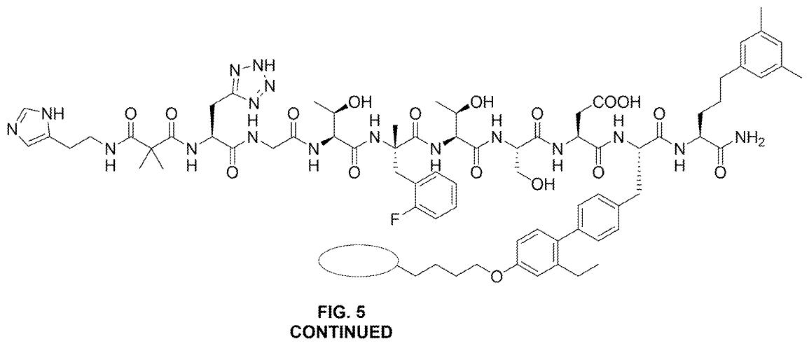

The drawings of this application relate to the drawings of WO 2022056494 (PCT/US2021/050337), which are hereby incorporated by reference.

### DETAILED DESCRIPTION

The present disclosure provides, in some aspects, antibody-drug conjugates that specifically bind the glucagon-like peptide 1 receptor (GLP1R) protein. As described in the Background section above, GLP1R and its ligand GLP1 are highly validated targets for obesity and type 2 diabetes. However, no direct agonist antibodies have been identified for type 2 diabetes treatment. Single peptides with agonist activities on GLP1R are effective therapeutic agents for glucose control and body weight loss, but in-line peptide-antibody fusions are susceptible to proteolysis. In certain embodiments of the present disclosure, antibody-drug conjugates were generated that combine an antibody, or antigen-binding fragment thereof, specifically targeting the extracellular domain of GLP1R, with a GLP1 peptidomimetic functionally activating GLP1R. In certain embodiments, antibody-drug conjugates of the present disclosure have a longer drug duration with comparable or better weight and glucose reducing efficacy and minimized off-target side effects.

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

### Definitions

Unless defined otherwise, all technical and scientific terms used herein have the same meaning as commonly understood by one of ordinary skill in the art to which this disclosure belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

The phrase “pharmaceutically acceptable salt”, as used in connection with compositions of the disclosure, refers to any salt suitable for administration to a patient. Suitable salts include, but are not limited to, those disclosed in. Berge et al., “Pharmaceutical Salts”, J. Pharm. Sci., 1977, 66:1, incorporated herein by reference. Examples of salts include, but are not limited to, acid derived, base derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, p toluene sulfonic acid, salicylic acid, and the like. In some examples, a payload described herein comprises a tertiary amine, where the nitrogen atom in the tertiary amine is the atom through which the payload is bonded to a linker or a linker-spacer. In such instances, bonding to the tertiary amine of the payload yields a quaternary amine in the linker-payload molecule. The positive charge on the quaternary amine can be balanced by a counter ion (e.g., chloro, bromo, iodo, or any other suitably charged moiety such as those described herein).

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present disclosure, such as payloads and linker-payloads, include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “halogen” means F, Cl, Br, or I; the term “halide” refers to a halogen radical or substituent, namely —F, —Cl, —Br, or —I.

The term “adduct”, e.g., “a Diels-Alder adduct” of the present disclosure encompasses any moiety comprising the product of an addition reaction, e.g., a Diels-Alder reaction, independent of the synthetic steps taken to produce the moiety.

The term “covalent attachment” means formation of a covalent bond, i.e., a chemical bond that involves sharing of one or more electron pairs between two atoms. Covalent bonding may include different interactions, including but not limited to σ-bonding, π-bonding, metal-to-metal bonding, agostic interactions, bent bonds, and three-center two-electron bonds. When a first group is said to be “capable of covalently attaching” to a second group, this means that the first group is capable of forming a covalent bond with the second group, directly or indirectly, e.g., through the use of a catalyst or under specific reaction conditions. Non-limiting examples of groups capable of covalently attaching to each other may include, e.g., an amine and a carboxylic acid (forming an amide bond), a diene and a dienophile (via a Diels-Alder reaction), a maleimide and a thiol (forming a thio-maleimide), and an azide and an alkyne (forming a triazole via a 1,3-cycloaddition reaction).

As described herein, compounds of the disclosure (e.g., payloads and linker-payloads) may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds.

The term “stable,” as used herein in reference to compounds, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

Unless otherwise stated, cyclic adducts, e.g., products of a cycloaddition reaction, e.g., an azide-acetylene cycloaddition reaction or a Diels-Alder reaction, depicted herein include all regioisomers, i.e., structural isomers that differ only in the position of a functional group or a substituent. By way of an example, the following structures represent triazole regioisomers, which differ only in the position of the substituent on the triazole ring:

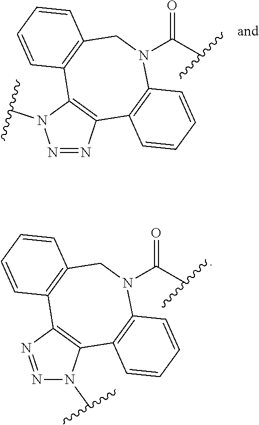 [SMILES: *C(=O)N1Cc2ccccc2-c2c(nnn2*)-c2ccccc21 ; *C(=O)N1Cc2ccccc2-c2nnn(*)c2-c2ccccc21]

Triazole regioisomers may also be represented by the following structure:

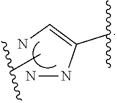

Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this disclosure.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the disclosure and salts thereof are also within the scope of the disclosure. The compounds of the disclosure may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” is meant that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

Certain groups, moieties, substituents, and atoms are depicted with a wavy line. The wavy line can intersect or cap a bond or bonds. The wavy line indicates the atom through which the groups, moieties, substituents, or atoms are bonded. For example, a phenyl group that is substituted with a propyl group depicted as:

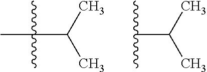

has the following structure:

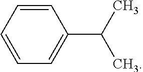

The term “GLP1R” refers to the glucagon-like peptide 1 receptor and includes recombinant GLP1R protein or a fragment thereof. GLP1R has a sequence of 463 residues. Donnelly, Br J Pharmacol, 166(1):27-41 (2011). Glucagon-like peptide 1 (GLP1) is a 31-amino acid peptide hormone released from intestinal L cells following nutrient consumption. The binding of GLP1 to GLP1R potentiates glucose-induced secretion of insulin from pancreatic beta cells, increases insulin expression, inhibits beta-cell apoptosis, promotes beta-cell neogenesis, reduces glucagon secretion, delays gastric emptying, promotes satiety and increases peripheral glucose disposal.

An antibody-tethered drug conjugate (ATDC) or antibody-drug conjugate (ADC) refers to an antibody or antigen-binding fragments thereof tethered, by a linker or without a linker, to a payload (e.g., a GLP1 peptidimimetic). An antibody-payload conjugate refers to such an antibody or fragment linked to a payload whereas an antibody-linker-payload conjugate refers to an antibody or fragment conjugated to a payload via a linker. An antibody or antigen-binding fragment referred to herein includes embodiments wherein said antibody or fragment is be conjugated to a payload or linker-payload.

### Anti-GLP1R Antigen-Binding Proteins and Conjugates

The present invention provides antigen-binding proteins, such as antibodies and antigen-binding fragments thereof, that bind specifically to GLP1R which may be conjugated to a payload, e.g., by a linker (a linker-payload) (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32). In an embodiment of the invention, the anti-GLP1R antibody or antigen-binding fragment tethered to a payload has the following structure:

BA-(L-P)ₘ (A),

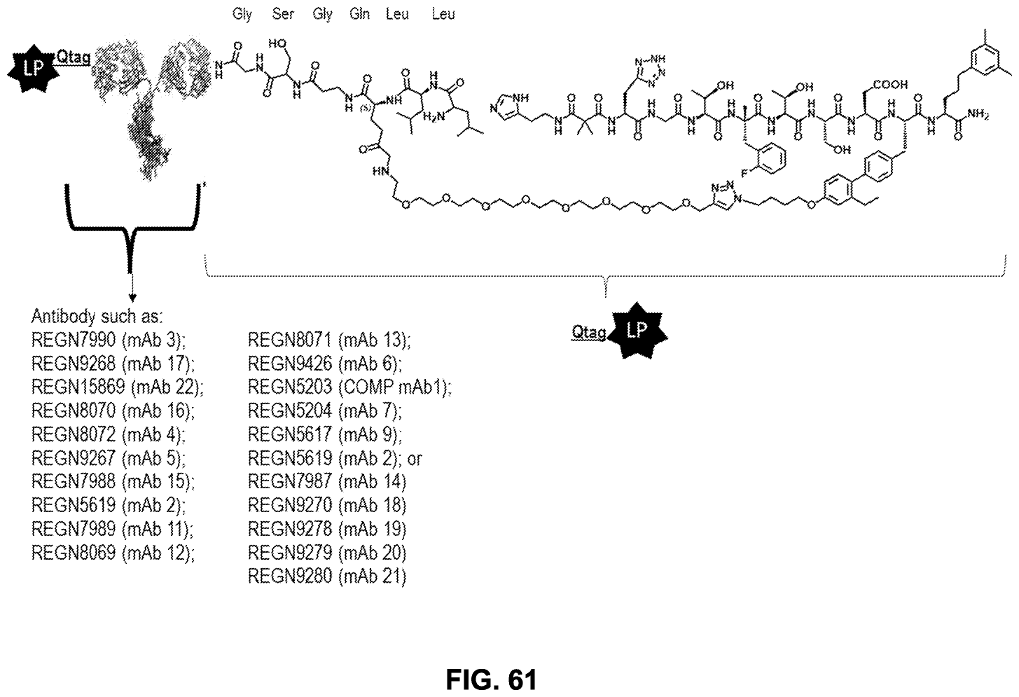

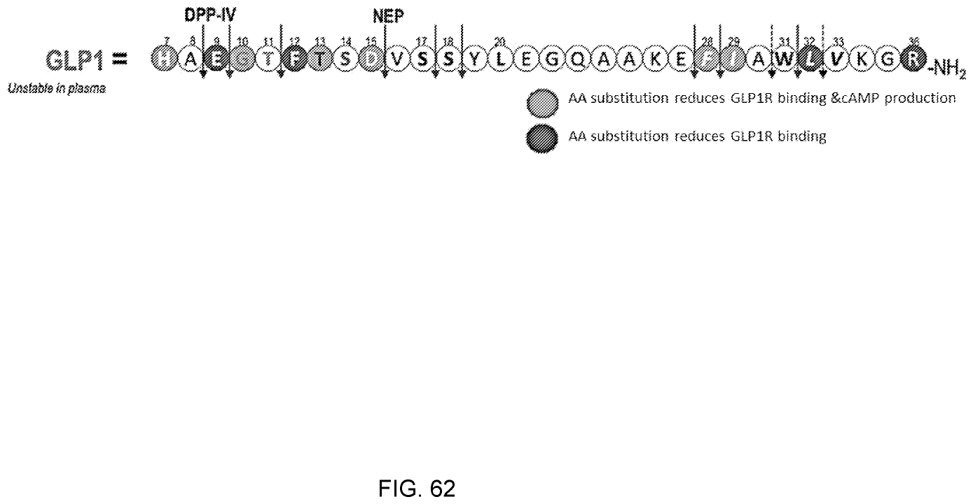

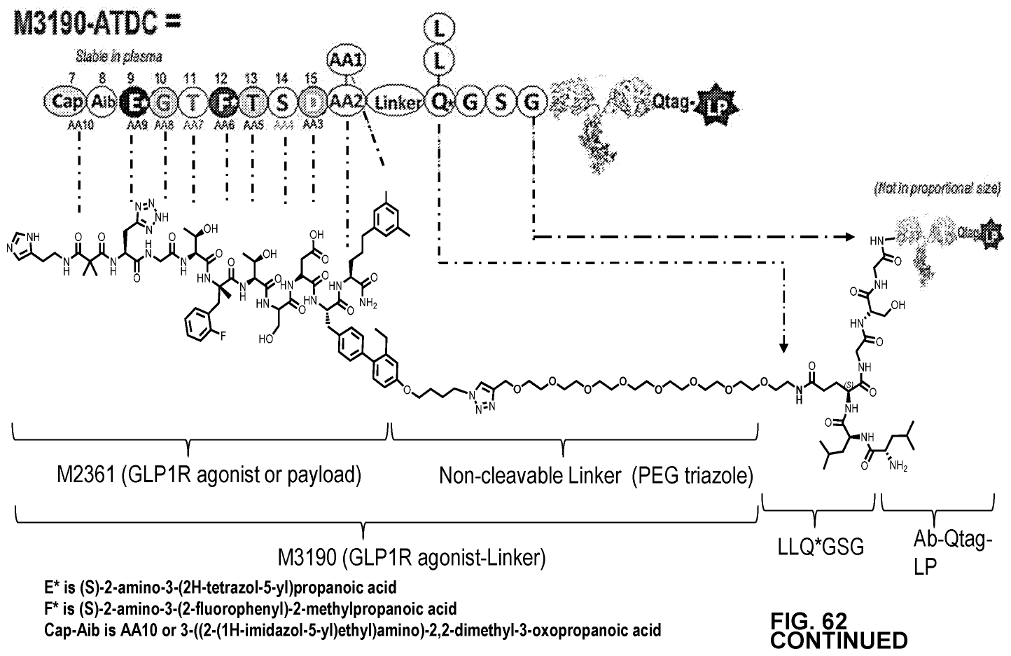

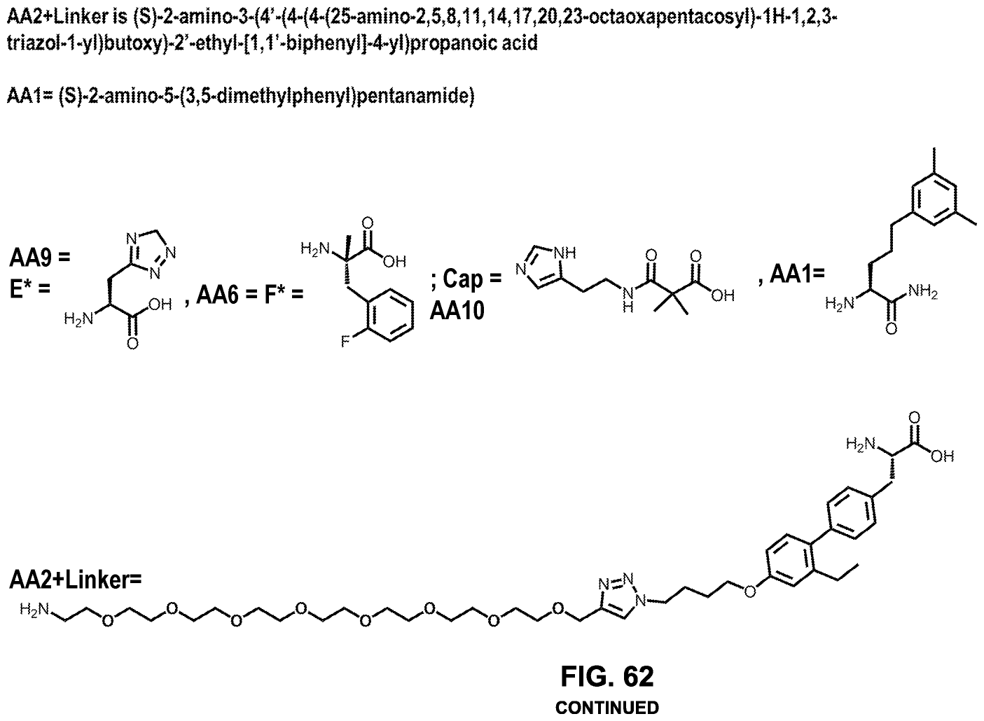

BA-L-P (I)

wherein:
BA is the anti-GLP1R antibody or antigen-binding fragment thereof:
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) which is an antibody or antigen-binding fragment thereof that competes for binding to GLP1R with said antibody or fragment of (i); and/or
(iii) which is an antibody or antigen-binding fragment thereof that binds to the same epitope of GLP1R as said antibody or fragment of (i);
L is a non-cleavable linker;
P is the payload (e.g., a drug payload such as a GLP1 peptidomimetic); and
m is 1, 2, 3 or 4.
   See for example, the ATDC in FIG. 61.

An antibody or antigen-binding fragment thereof or conjugate thereof that specifically binds GLP1R may be referred to as “anti-GLP1R”. Anti-GLP1R antibodies and antigen-binding fragments thereof refer to antibodies and fragments that bind to GLP1R with a K_{D} of about 1-2 nM or a greater affinity.

An “agonist” antibody or antigen-binding fragment thereof (e.g., an ATDC) as used herein is an antibody or fragment that increases or enhances at least one biological activity of GLP1R. Such increase or enhancement may be mediated by the antibody itself or by the payload or linker-payload of an ATDC. For example, the agonist antibody or fragment may elicit stimulation of the adenylate cyclase pathway resulting in increased synthesis of cyclic AMP and release of insulin if the cell is a mammalian pancreatic beta cell. Other biological activities of GLP1R may be cAMP-dependent activation of protein kinase A (PKA) and/or cAMP-regulated guanine nucleotide exchange factor 2 (Epac2). An agonist antibody or fragment may also reduce glucose levels or reduce body weight upon administration to a subject in need thereof.

A “neutral” antibody or a “neutral” binder with respect to an anti-GLP1R antibody or antigen-binding fragment thereof refers to an antibody or fragment that binds to GLP1R but does not significantly activate biological activity of GLP1R (e.g., stimulation of adenylate cyclase pathway).

All amino acid abbreviations used in this disclosure are those accepted by the United States Patent and Trademark Office as set forth in 37 C.F.R. § 1.822 (B)(J).

The term “protein” or “polypeptide” means any amino acid polymer having amino acids covalently linked via peptide bonds. “Protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins can be produced using recombinant cell-based production systems, such as the insect bacculovirus system, yeast systems (e.g., Pichia sp, such as Pichia pastoris), mammalian systems (e.g., CHO cells and CHO derivatives like CHO—K1 cells).

A polynucleotide includes DNA and RNA.

“GLP1R” means human GLP1R unless specified as being from a non-human species, e.g., “mouse GLP1R,” “monkey GLP1R,” etc.

The amino acid sequence of an antibody or antigen-binding fragment thereof can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). In an embodiment of the invention, the CDRs of an anti-GLP1R antibody or antigen-binding fragment (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) heavy or light chain immunoglobulin are as defined by Kabat, Chohia, Contact, IMGT or AHo.

The anti-GLP1R antibodies and antigen-binding fragments of the present invention may be glutaminyl-modified. The term “glutaminyl-modified” antibody, for example, refers to an antibody (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) with at least one covalent linkage from a glutamine side chain (from the glutamine (Q) residue in LLQGSG (SEQ ID NO: 18)) to a primary amine compound (e.g., a Payload or Linker-Payload) of the present disclosure. In particular embodiments of the invention, the primary amine compound is linked through an amide linkage on the glutamine side chain. In certain embodiments of the invention, the glutamine is an endogenous glutamine. In other embodiments of the invention, the glutamine is an endogenous glutamine made reactive by polypeptide engineering (e.g., via amino acid deletion, insertion, substitution, or mutation on the polypeptide). In additional embodiments of the invention, the glutamine is polypeptide engineered with an acyl donor glutamine-containing tag (e.g., glutamine-containing peptide tags, Q-tags or TGase recognition tag).

Transglutaminase (TGase) is an enzyme that catalyzes transamidation reactions of glutamine (Q) residues in a recognition sequence (the ‘Q-tag’ or “Qtag” or “TGase recognition tag”) over other glutamines, e.g., in heavy chains of IgGs, thus facilitating site-specific modification. In an embodiment of the invention, the antibody or antigen-binding fragment thereof has been modified to comprise a TGase recognition tag. Suitable TGase recognition tags include those described herein. In an embodiment of the invention, the TGase is microbial transglutaminase, e.g., Streptomyces transglutaminase. Sarafeddinov, A Novel Transglutaminase Substrate from Streptomyces mobaraensis Inhibiting Papain-Like Cysteine Proteases”, J. Microbiol. Biotechnol. 2011, 21:617-26. In an embodiment of the invention, a transglutaminase joins an amine to glutamine (Gln, Q) (e.g., in a Qtag having the amino acid sequence LLQGSG (SEQ ID NO: 18)) according to the following reaction scheme: Gln(C═O)NH₂+RNH₂→Gln(C═O)NHR+NH₃; e.g., wherein RNH₂ includes H₂N—((CH₂)₂—O)ₙ—.

The term “TGase recognition tag” refers to a sequence of amino acids comprising an acceptor glutamine residue and that when incorporated into (e.g. appended to) a polypeptide sequence, under suitable conditions, is recognized by a TGase (transglutaminase) and leads to cross-linking by the TGase through a reaction between an amino acid side chain within the sequence of amino acids and a reaction partner. The recognition tag may be a peptide sequence that is not naturally present in the polypeptide comprising the TGase recognition tag. In some embodiments of the invention, the TGase recognition tag comprises at least one Gln. In some embodiments of the invention, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Met, Pro, Thr, Lys, or Trp or nonconventional amino acid). In some embodiments of the invention, the acyl donor glutamine-containing tag comprises an amino acid sequence selected from the group consisting of LLQGG (SEQ ID NO: 6), LLQG (SEQ ID NO: 7), LSLSQG (SEQ ID NO: 8), GGGLLQGG (SEQ ID NO: 9), GLLQG (SEQ ID NO: 10), LLQ, GSPLAQSHGG (SEQ ID NO: 11), GLLQGGG (SEQ ID NO: 12), GLLQGG (SEQ ID NO: 13), GLLQ (SEQ ID NO: 14), LLQLLQGA (SEQ ID NO: 15), LLQGA (SEQ ID NO: 16), LLQYQGA (SEQ ID NO: 17), LLQGSG (SEQ ID NO: 18), LLQYQG (SEQ ID NO: 19), LLQLLQG (SEQ ID NO: 20), SLLQG (SEQ ID NO: 21), LLQLQ (SEQ ID NO: 22), LLQLLQ (SEQ ID NO: 23), LLQGSGSG (SEQ ID NO: 185) and LLQGR (SEQ ID NO: 24). See for example, International patent application publication no. WO2012/059882 or U.S. patent Ser. No. 10/842,881, the entire contents of which are incorporated by reference herein.

In certain embodiments, the antibody or antigen-binding fragment thereof has been modified to comprise a Q-tag at the N-terminus of one or both of the antibody or fragment light chains. In certain embodiments, the antibody or fragment thereof has been modified to comprise a Q-tag at the N-terminus of both antibody light chains.

In certain embodiments, the antibody or antigen-binding fragment thereof has been modified to comprise a Q-tag at the N-terminus of one or both antibody or antigen-binding fragment heavy chains. In certain embodiments, the antibody or antigen-binding fragment thereof has been modified to comprise a Q-tag at the N-terminus of both antibody heavy chains.

In certain embodiments, the antibody or antigen-binding fragment thereof has been modified to comprise a Q-tag at the C-terminus of one or both antibody or antigen-binding fragment light chains. In certain embodiments, the antibody or antigen-binding fragment thereof has been modified to comprise a Q-tag at the C-terminus of both antibody light chains.

In certain embodiments, the antibody or antigen-binding fragment thereof has been modified to comprise a Q-tag at the C-terminus of one or both antibody or antigen-binding fragment heavy chains. In certain embodiments, the antibody or antigen-binding fragment thereof has been modified to comprise a Q-tag at the C-terminus of both antibody heavy chains.

The term “antibody” refers to immunoglobulin molecules comprising four polypeptide chains, two heavy chains (HC) and two light chains (LC) inter-connected by disulfide bonds, as well as multimers thereof (e.g., IgM). Preferably, the antibody is an IgG format (e.g., IgG1, IgG2, IgG3 or IgG4 or a variant thereof, for example, IgG4 having an S228P mutation) having the 2 heavy chains and 2 light chains interconnected by disulfide bonds to form a tetramer with a Y-like shape. See Silva et al., The S228P Mutation Prevents in Vivo and in Vitro IgG4 Fab-arm Exchange as Demonstrated using a Combination of Novel Quantitative Immunoassays and Physiological Matrix Preparation, THE JOURNAL OF BIOLOGICAL CHEMISTRY VOL. 290, NO. 9, pp. 5462-5469 (2015). In an embodiment of the invention, the antibody or antigen-binding fragment is an IgA, IgD, IgE, IgM, IgA1 or IgA2 (or a variant thereof). An antibody may be conjugated to a payload, e.g., by a linker.

The antibody or antigen-binding fragment can be in any form known to those of skill in the art. In certain embodiments, the antibody or fragment comprises a light chain. In certain embodiments, the light chain is a kappa light chain. In certain embodiments, the light chain is a lambda light chain.

Each heavy chain comprises a heavy chain variable region (abbreviated herein as HCVR or V_{H}) and a heavy chain constant region (e.g., a human heavy chain constant region). Each light chain comprises a light chain variable region (abbreviated herein as LCVR or V_{L}) and a light chain constant region (e.g., a human light chain constant region). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding portion thereof) can be identical to the human germline sequences, or can be naturally or artificially modified. An amino acid consensus sequence can be defined based on a side-by-side analysis of two or more CDRs.

Antigen-binding fragments of antibodies that bind specifically to GLP1R are also part of the present invention. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody can be derived, e.g., from antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc. An antigen-binding fragment may be conjugated to a payload, e.g., by a linker.

Non-limiting examples of antigen-binding fragments include: (i) Fab fragments; (ii) F(ab′)₂ fragments; (iii) Fd fragments; (iv) Fv fragments; (v) single-chain Fv (scFv) or scFv-Fc molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. In some aspects of the invention, the antibody fragment is an In some aspects, the antibody fragment is a Fab′ fragment. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody may include at least one variable domain. The variable domain can be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains can be situated relative to one another in any suitable arrangement. For example, the variable region can be dimeric and contain V_{H}-V_{H}, V_{H}-V_{L} or V_{L}-V_{L} dimers.

Alternatively, the antigen-binding fragment of an antibody can contain a monomeric V_{H} or V_{L} domain.

An antigen-binding fragment of an antibody can contain at least one variable domain covalently linked to at least one constant domain. Non-limiting, exemplary configurations of variable and constant domains that can be found within an antigen-binding fragment of an antibody of the present description include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed herein, the variable and constant domains can be either directly linked to one another or can be linked by a full or partial hinge or linker region. A hinge region can consist of at least 2 (e.g., 5, 10, 15, 20, 40, 60, or more) amino acids which result in a flexible or semi-flexible linkage between adjacent variable and/or constant domains in a single polypeptide molecule.

Moreover, an antigen-binding fragment of an antibody of the present description can comprise a homo-dimer or hetero-dimer (or other multimer) of any of the variable and constant domain configurations listed herein in non-covalent association with one another and/or with one or more monomeric V_{H} or V_{L} domain (e.g., by disulfide bond(s)).

As with antibodies, antigen-binding fragments can be monospecific or multispecific (e.g., bispecific). A multispecific antigen-binding fragment of an antibody may comprise at least two different variable domains, wherein each variable domain is capable of specifically binding to a separate antigen or to a different epitope on the same antigen. Any multispecific antibody format, including the exemplary bispecific antibody formats disclosed herein, can be adapted for use in the context of an antigen-binding fragment of an antibody of the present description using routine techniques available in the art.

In certain embodiments, the antibodies of the description, e.g., anti-GLP1R antibodies, are human antibodies. The term “human antibody,” as used herein, is intended to include antibodies having variable and constant regions derived from human germline immunoglobulin sequences. The human antibodies of the description can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, that have been grafted onto human framework sequences.

In an embodiment of the invention, the antibody is a monoclonal antibody. In an embodiment of the invention, the antibody is a polyclonal antibody. In an embodiment of the invention, the antibody is a chimeric antibody. In an embodiment of the invention, the antibody is a humanized antibody.

The antibodies and antigen-binding fragments can, in some embodiments, be recombinant antibodies and antigen-binding fragments. The term “recombinant” antibody as used herein, is intended to include antibodies that are prepared, expressed, created or isolated by recombinant means. For example, recombinant antibodies include those expressed using a recombinant expression vector transfected into a host cell (e.g., a Chinese hamster ovary cell) which is optionally isolated from the host cell and/or culture media in which the host cell is grown. Recombinant antibodies include those isolated from a recombinant, combinatorial human antibody library, antibodies isolated from an animal (e.g., a mouse) that is transgenic for human immunoglobulin genes (See, e.g., Taylor et al. (1992) Nucl. Acids Res. 20:6287-6295) or antibodies prepared, expressed, created or isolated by any other means that involves splicing of human immunoglobulin gene sequences to other DNA sequences. Such human antibodies have variable and constant regions derived from human germline immunoglobulin sequences. In certain embodiments, however, such human antibodies are subjected to in vitro mutagenesis (or, when an animal transgenic for human Ig sequences is used, in vivo somatic mutagenesis) and thus the amino acid sequences of the V_{H} and V_{L} regions of the antibodies are sequences that, while derived from and related to human germline V_{H} and V_{L} sequences, may not naturally exist within the human antibody germline repertoire in vivo.

The antibodies and antigen-binding fragments of the description can be isolated or purified antibodies. An “isolated” or “purified” antibody, as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present description. Moreover, an antibody that is removed partially or fully from a recombinant host cell in which is it produced is “isolated”. For example, an antibody that has been purified from at least one component of a reaction or reaction sequence, is an “isolated” or “purified” antibody. An isolated antibody also includes an antibody in situ within a recombinant cell. Isolated antibodies include those that have been subjected to at least one purification or isolation step. According to certain embodiments, an isolated antibody can be substantially free of other cellular material and/or chemicals.

The antibodies and antigen-binding fragments disclosed herein can comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present description includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with given heavy and light chain variable region sequences, can produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies and antigen-binding fragments of the present description can contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be tested for one or more desired property such as, improved binding specificity, increased binding affinity, improved or enhanced antagonistic or agonistic biological properties (as the case may be), reduced immunogenicity, improved drug-to-antibody ratio (DAR) for antibody-drug conjugates, etc. Antibodies and antigen-binding fragments obtained in this general manner are encompassed within the present description.

The present invention includes anti-GLP1R antibodies and antigen-binding fragments thereof (e.g., which are conjugated to a payload, e.g., by a linker) that are aglycosylated. The term “aglycosylated” antibody or antigen-binding fragment includes an antibody or fragment that does not comprise a glycosylation sequence that might interfere with a transglutamination reaction, for example, an antibody that does not have saccharide group at N297 on one or more heavy chains. In particular embodiments, an antibody heavy chain has an N297 mutation. The antibody can be mutated to no longer have an asparagine residue at position 297 according to the EU numbering system as disclosed by Kabat et al. In particular embodiments, an antibody heavy chain has an N297Q or an N297D mutation. Such an antibody can be prepared by site-directed mutagenesis to remove or disable a glycosylation sequence or by site-directed mutagenesis to insert a glutamine residue at site apart from any interfering glycosylation site or any other interfering structure. Such an antibody also can be isolated from natural or artificial sources. Aglycosylated antibodies and fragments also include antibodies and fragments comprising a T299 or S298P or other mutations, or combinations of mutations that result in a lack of glycosylation. An aglycosylated antibody or antigen-binding fragment may be completely lacking glycosylation, e.g., following expression in a bacterial host cell.

The present invention includes anti-GLP1R antibodies and antigen-binding fragments thereof (e.g., which are conjugated to a payload, e.g., by a linker) that are deglycosylated. The term “deglycosylated” antibody or antigen-binding fragment refers to an antibody or fragment in which a saccharide group at is removed to facilitate transglutaminase-mediated conjugation. Saccharides include, but are not limited to, N-linked oligosaccharides. In some embodiments, deglycosylation is performed at residue N297. In some embodiments, removal of saccharide groups is accomplished enzymatically, included but not limited to via PNGase.

The term “epitope” refers to an antigenic determinant (e.g., of GLP1R) that interacts with a specific antigen binding site in the variable region of an antibody or antigen-binding fragment known as a paratope. A single antigen can have more than one epitope. Thus, different antibodies can bind to different areas on an antigen and can have different biological effects. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope can include moieties of saccharides, phosphoryl groups, or sulfonyl groups on the antigen.

The terms “conjugated” protein, antibody or antigen-binding fragment as used herein refers to a protein, antibody or fragment covalently linked to one or more chemical moieties. The chemical moiety can include an amine compound of the present disclosure. Linkers (L) and payloads (P) suitable for use with the present disclosure are described in detail herein.

The term “Drug-to-Antibody Ratio” or (DAR) is the average number of therapeutic moieties, e.g., drugs, conjugated to a binding agent (e.g., an antibody or antigen-binding fragment) of the present disclosure.

The term “Linker Antibody Ratio” or (LAR), also denoted as the lower case, in some embodiments, is the average number of reactive primary amine compounds conjugated to a binding agent of the present disclosure. Such binding agents, e.g., antibodies or antigen-binding fragments, can be conjugated with primary amine compounds comprising, e.g., a suitable azide or alkyne. The resulting binding agent, which is functionalized with an azide or an alkyne can subsequently react with a therapeutic moiety comprising the corresponding azide or alkyne via the 1,3-cycloaddition reaction.

The phrase “reaction pH” refers to the pH of a reaction after all reaction components or reactants have been added.

A “variant” of a polypeptide, such as an immunoglobulin chain (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280 V_{H}, V_{L}, HC or LC; or CDR thereof as set forth herein), refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 26; 28; 30; 32; 34; 36; 40; 42; 44; 46; 48; 50; 52; 54; 56; 60; 62; 64; 66; 68; 70; 72; 74; 76; 80; 82; 414; 416; 84; 86; 88; 90; 92; 94; 96; 100; 102; 104; 106; 108; 110; 112; 114; 116; 120; 122; 124; 126; 128; 130; 132; 134; 136; 140; 142; 144; 146; 148; 150; 152; 154; 156; 160; 162; 164; 166; 168; 170; 172; 174; 176; 180; 182; 184; 187; 189; 191; 193; 195; 197; 201; 203; 205; 207; 209; 211; 213; 215; 217; 221; 223; 225; 227; 229; 231; 233; 235; 237; 241; 243; 245; 247; 249; 251; 253; 255; 257; 261; 263; 265; 267; 269; 271; 273; 275; 277; 279; 281; 283; 285; 289; 291; 293; 295; 297; 299; 301; 303; 305; 309; 311; 313; 315; 317; 319; 321; 323; 325; 329; 331; 333; 335; 337; 339; 341; 343; 345; 349; 351; 353; 355; 357; 359; 361; 363; 365; 369; 371; 373; 375; 377; 379; 381; 383; 385; 389; 391; 393; 395; 397; 399; 401; 403; 405; 409; 411; or 413; or GAS, AAS or KIS); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (e.g., any of SEQ ID NOs: 25; 27; 29; 31; 33; 35; 39; 41; 43; 45; 47; 49; 51; 53; 55; 59; 61; 63; 65; 67; 69; 71; 73; 75; 79; 81; 415; 417; 83; 85; 87; 89; 91; 93; 95; 99; 101; 103; 105; 107; 109; 111; 113; 115; 119; 121; 123; 125; 127; 129; 131; 133; 135; 139; 141; 143; 145; 147; 149; 151; 153; 155; 159; 161; 163; 165; 167; 169; 171; 173; 175; 179; 181; 183; 186; 188; 190; 192; 194; 196; 200; 202; 204; 206; 208; 210; 212; 214; 216; 220; 222; 224; 226; 228; 230; 232; 234; 236; 240; 242; 244; 246; 248; 250; 252; 254; 256; 260; 262; 264; 266; 268; 270; 272; 274; 276; 278; 280; 282; 284; 288; 290; 292; 294; 296; 298; 300; 302; 304; 308; 310; 312; 314; 316; 318; 320; 322; 324; 328; 330; 332; 334; 336; 338; 340; 342; 344; 348; 350; 352; 354; 356; 358; 360; 362; 364; 368; 370; 372; 374; 376; 378; 380; 382; 384; 388; 390; 392; 394; 396; 398; 400; 402; 404; 408; 410; or 412 or GGTGCATCC, GCTGCATCC or AAGATTTCT); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

Anti-GLP1R antigen-binding proteins, e.g., antibodies and antigen-binding fragments thereof of the present invention, in an embodiment of the invention, include a heavy chain immunoglobulin or variable region thereof having at least 70% (e.g., 80%, 85%, 90%, 95%, 99%) amino acid sequence identity to the amino acids set forth in SEQ ID NO: 26, 46, 66, 86, 106, 126, 146, 166, 42, 62, 82, 414; 416; 102, 122, 142, 162 or 182; and/or a light chain immunoglobulin or variable region thereof having at least 70% (e.g., 80%, 85%, 90%, 95%, 99%) amino acid sequence identity to the amino acids set forth in SEQ ID NO: 34, 54, 74, 94, 114, 134, 154, 174, 44, 64, 84, 104, 124, 144, 164 or 184.

In addition, a variant of a polypeptide may include an amino acid sequence that is set forth herein (e.g., SEQ ID NO: 26, 28, 30, 32, 34, 36, 40, 42, 44, 46, 48, 50, 52, 54, 56, 60, 62, 64, 66, 68, 70, 72, 74, 76, 80, 82, 414; 416; 84, 86, 88, 90, 92, 94, 96, 100, 102, 104, 106, 108, 110, 112, 114, 116, 120, 122, 124, 126, 128, 130, 132, 134, 136, 140, 142, 144, 146, 148, 150, 152, 154, 156, 160, 162, 164, 166, 168, 170, 172, 174, 176, 180, 182 or 184 or GAS, AAS or KIS) except for one or more (e.g., 1, 2, 3, 4, 5, 6, 7, 8, 9 or 10) mutations such as, for example, missense mutations (e.g., conservative substitutions), non-sense mutations, deletions, or insertions. For example, the present invention includes anti-GLP1R antibodies and antigen-binding fragments thereof which include an immunoglobulin light chain (or V_{L}) variant comprising the amino acid sequence set forth in SEQ ID NO: 34, 54, 74, 94, 114, 134, 154, 174, 44, 64, 84, 104, 124, 144, 164 or 184 but having one or more of such mutations and/or an immunoglobulin heavy chain (or V_{H}) variant comprising the amino acid sequence set forth in SEQ ID NO: 26, 46, 66, 86, 106, 126, 146, 166, 42, 62, 82, 414; 416; 102, 122, 142, 162 or 182 but having one or more of such mutations. In an embodiment of the invention, an anti-GLP1R antibody or antigen-binding fragment thereof includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

Embodiments of the present invention also include antigen-binding proteins, e.g., anti-GLP1R antibodies and antigen-binding fragments thereof, that comprise immunoglobulin VHs and VLs; or HCs and LCs, which comprise a variant amino acid sequence having 70% or more (e.g., 80%, 85%, 90%, 95%, 97% or 99%) overall amino acid sequence identity or similarity to the amino acid sequences of the corresponding VHs, VLs, HCs or LCs specifically set forth herein, but wherein the CDR-L1, CDR-L2, CDR-L3, CDR-H1, CDR-H2 and CDR-H3 of such immunoglobulins are not variants and comprise the amino acid sequences specifically set forth herein. Thus, in such embodiments, the CDRs within variant antigen-binding proteins are not, themselves, variants.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present invention includes anti-GLP1R antigen-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45.

Immunoglobulin chains of the anti-GLP1R antibodies and antigen-binding fragments of the present invention are summarized below in Tables A and B.

TABLE A
Anti-GLP1R Antibody and Antigen-binding Fragment Amino Acid Sequence Identifiers

| Antibody | | | | | | | | | | |
|---|---|---|---|---|---|---|---|---|---|---|
| Designation | HCVR | CDR-H1 | CDR-H2 | CDR-H3 | HC | LCVR | CDR-L1 | CDR-L2 | CDR-L3 | LC |
| REGN9268 | 26 | 28 | 30 | 32 | 42 | 34 | 36 | GAS | 40 | 44 |
| (mAb 17) | | | | | | | | | | |
| REGN7990 | 46 | 48 | 50 | 52 | 62 | 54 | 56 | AAS | 60 | 64 |
| (mAb 3) | | | | | | | | | | |
| REGN15869 | 66 | 68 | 70 | 72 | 82 | 74 | 76 | AAS | 80 | 84 |
| REGN18121- | 66 | 68 | 70 | 72 | 414 | 74 | 76 | AAS | 80 | 84 |
| REGN18123- | 66 | 68 | 70 | 72 | 416 | 74 | 76 | AAS | 80 | 84 |
| REGN8070 | 86 | 88 | 90 | 92 | 102 | 94 | 96 | KIS | 100 | 104 |
| (mAb 16) | | | | | | | | | | |
| REGN8072 | 106 | 108 | 110 | 112 | 122 | 114 | 116 | AAS | 120 | 124 |
| (mAb 4) | | | | | | | | | | |
| REGN9267 | 126 | 128 | 130 | 132 | 142 | 134 | 136 | GAS | 140 | 144 |
| (mAb 5) | | | | | | | | | | |
| REGN7988 | 146 | 148 | 150 | 152 | 162 | 154 | 156 | AAS | 160 | 164 |
| (mAb 15) | | | | | | | | | | |
| REGN5619 | 166 | 168 | 170 | 172 | 182 | 174 | 176 | AAS | 180 | 184 |
| (mAb 2) | | | | | | | | | | |
| REGN7989 | 187 | 189 | 191 | 193 | 203 | 195 | 197 | AAS | 201 | 205 |
| (mAb 11) | | | | | | | | | | |
| REGN8069 | 207 | 209 | 211 | 213 | 223 | 215 | 217 | KIS | 221 | 225 |
| (mAb 12) | | | | | | | | | | |
| REGN8071 | 227 | 229 | 231 | 233 | 243 | 235 | 237 | AAS | 241 | 245 |
| (mAb 13) | | | | | | | | | | |
| REGN9426 | 247 | 249 | 251 | 253 | 263 | 255 | 257 | AAS | 261 | 265 |
| (mAb 6) | | | | | | | | | | |
| REGN5203 | | | | | 267 | | | | | 269 |
| (COMP | | | | | | | | | | |
| mAb 1) | | | | | | | | | | |
| REGN5204 | | | | | 271 | | | | | 273 |
| (mAb 7) | | | | | | | | | | |
| REGN5617 | 275 | 277 | 279 | 281 | 291 | 283 | 285 | AAS | 289 | 293 |
| (mAb 9; | | | | | | | | | | |
| mAb 10) | | | | | | | | | | |
| REGN5619 | 295 | 297 | 299 | 301 | 311 | 303 | 305 | AAS | 309 | 313 |
| (mAb 2) | | | | | | | | | | |
| REGN7987 | 315 | 317 | 319 | 321 | 331 | 323 | 325 | AAS | 329 | 333 |
| (mAb 14) | | | | | | | | | | |
| REGN9270 | 335 | 337 | 339 | 341 | 351 | 343 | 345 | GAS | 349 | 353 |
| (mAb 18) | | | | | | | | | | |
| REGN9278 | 355 | 357 | 359 | 361 | 371 | 363 | 365 | GAS | 369 | 373 |
| (mAb 19) | | | | | | | | | | |
| REGN9279 | 375 | 377 | 379 | 381 | 391 | 383 | 385 | GAS | 389 | 393 |
| (mAb 20) | | | | | | | | | | |
| REGN9280 | 395 | 397 | 399 | 401 | 411 | 403 | 405 | GAS | 409 | 413 |
| (mAb 21) | | | | | | | | | | |

HC = Immunoglobulin heavy chain; LC = Immunoglobulin light chain; HCVR = Heavy chain variable region; LCVR = Light chain variable region. Optionally, any HCVR and/or LCVR set forth herein includes an N-terminal Qtag such as LLQGSG (SEQ ID NO: 18). Optionally, any light chain and/or heavy chain set forth herein does not include an N-terminal Qtag such as LLQGSG (SEQ ID NO: 18).

TABLE B
Anti-GLP1R Antibody and Antigen-binding Fragment Nucleotide Sequence
Identifiers

| Antibody | | CDR- | CDR- | CDR- | | | CDR- | | CDR- | |
|---|---|---|---|---|---|---|---|---|---|---|
| Designation | HCVR | H1 | H2 | H3 | HC | LCVR | L1 | CDR-L2 | L3 | LC |
| REGN92658 | 25 | 27 | 29 | 31 | 41 | 33 | 35 | GGTGCATCC | 39 | 43 |
| REGN7990 | 45 | 47 | 49 | 51 | 61 | 53 | 55 | GCTGCATCC | 59 | 63 |
| REGN15869 | 65 | 67 | 69 | 71 | 81 | 73 | 75 | GCTGCATCC | 79 | 83 |
| REGN18121- | 65 | 67 | 69 | 71 | 415 | 73 | 75 | GCTGCATCC | 79 | 83 |
| REGN18123- | 65 | 67 | 69 | 71 | 417 | 73 | 75 | GCTGCATCC | 79 | 83 |
| REGN8070 | 85 | 87 | 89 | 91 | 101 | 93 | 95 | AAGATTTCT | 99 | 103 |
| REGN8072 | 105 | 107 | 109 | 111 | 121 | 113 | 115 | GCTGCATCC | 119 | 123 |
| REGN9267 | 125 | 127 | 129 | 131 | 141 | 133 | 135 | GGTGCATCC | 139 | 143 |
| REGN7988 | 145 | 147 | 149 | 151 | 161 | 153 | 155 | GCTGCATCC | 159 | 163 |
| REGN5619 | 165 | 167 | 169 | 171 | 181 | 173 | 175 | GCTGCATCC | 179 | 183 |
| REGN7989 | 186 | 188 | 190 | 192 | 202 | 194 | 196 | GCTGCATCC | 200 | 204 |
| REGN8069 | 206 | 208 | 210 | 212 | 222 | 214 | 216 | AAGATTTCT | 220 | 224 |
| REGN8071 | 226 | 228 | 230 | 232 | 242 | 234 | 236 | GCTGCATCC | 240 | 244 |
| REGN9426 | 246 | 248 | 250 | 252 | 262 | 254 | 256 | GCTGCATCC | 260 | 264 |
| REGN5203 | | | | | 266 | | | | | 268 |
| REGN5204 | | | | | 270 | | | | | 272 |
| REGN5617 | 274 | 276 | 278 | 280 | 290 | 282 | 284 | GCTGCATCC | 288 | 292 |
| REGN5619 | 294 | 296 | 298 | 300 | 310 | 302 | 304 | GCTGCATCC | 308 | 312 |
| REGN7987 | 314 | 316 | 318 | 320 | 330 | 322 | 324 | GCTGCATCC | 328 | 332 |
| REGN9270 | 334 | 336 | 338 | 340 | 350 | 342 | 344 | GGTGCATCC | 348 | 352 |
| REGN9278 | 354 | 356 | 358 | 360 | 370 | 362 | 364 | GGTGCATCC | 368 | 372 |
| REGN9279 | 374 | 376 | 378 | 380 | 390 | 382 | 384 | GGTGCATCC | 388 | 392 |
| REGN9280 | 394 | 396 | 398 | 400 | 410 | 402 | 404 | GGTGCATCC | 408 | 412 |

HC = Immunoglobulin heavy chain; LC = Immunoglobulin light chain; HCVR = Heavy chain variable
region; LCVR = Light chain variable region
REGN9268
Heavy chain variable region (HCVR; V_{H})-nucleotide sequence
GAAGTGCAACTGGTGGAGTCTGGGGGAGGCCTGGTCAAGCCTGGGGGGTCCCTGAGACTCTCCTGTGCAGCCTCTGGATT
CATCTTCAGTAGATATAGCATGAACTGGGTCCGCCAGGCTCCAGGGAAGGGGCTGGAGTGGGTCTCATCCATGAGTAGTA
ATAGTAAAAACACATACTACGCAGACTCAGTGAAGGGCCGATTCACCATCTCCAGAGACAACGCCAAAAACTCACTGTTT
CTGCAAATGAACACCCTGAGAGCCGAGGACACGGCTGTTTATTACTGTGCGAGAGATGGATACACCCTCAGGGCTTTTGA
TATCTGGGGCCAAGGGACAATGGTCACCGTCTCTTCA (SEQ ID NO: 25)
Heavy chain variable region (HCVR; V_{H})-amino acid sequence
EVQLVESGGGLVKPGGSLRLSCAASGFIFSRYSMNWVRQAPGKGLEWVSSMSSNSKNTYYADSVKGRFTISRDNAKNSLF
LQMNTLRAEDTAVYYCARDGYTLRAFDIWGQGTMVTVSS (SEQ ID NO: 26)
CDR-H1-nucleotide sequence
GGA TTC ATC TTC AGT AGA TAT AGC (SEQ ID NO: 27)
CDR-H1-amino acid sequence
G F I F S R Y S (SEQ ID NO: 28)
CDR-H2-nucleotide sequence
ATG AGT AGT AAT AGT AAA AAC ACA (SEQ ID NO: 29)
CDR-H2-amino acid sequence
M S S N S K N T (SEQ ID NO: 30)
CDR-H3-nucleotide sequence
GCG AGA GAT GGA TAC ACC CTC AGG GCT TTT GAT ATC (SEQ ID NO: 31)
CDR-H3-amino acid sequence
A R D G Y T L R A F D I (SEQ ID NO: 32)
Light chain variable region (LCVR; V_{L})-nucleotide sequence
GAAATTGTGTTGACGCAGTCTCCAGGCACCCTGTCTCTGTCTCCAGGGGAAAGAGACACCCTCTCCTGCAGGGCCAGTCA
GAGTATTGCCGGCAGATACGTAGCCTGGTACCAGCAGAAACCTGGCCAGGCACCCAGACTCCTCATCTACGGTGCATCCA
GCAGGGCCACTGGCATCCCAGACAGATTCAGTGGCAGTGGGTCTGGGACAGACTTCACTCTCACCATCAGCAGACTGGAG
CCTGAAGATTTTGCAGTGTATTACTGTCAGCAATATGGTAGCTCACCTTGGACGTTCGGCCAAGGGACCAAGGTGGAAAT
CAAA (SEQ ID NO: 33)
Light chain variable region (LCVR; V_{L})-amino acid sequence
EIVLTQSPGTLSLSPGERDTLSCRASQSIAGRYVAWYQQKPGQAPRLLIYGASSRATGIPDRFSGSGSGTDFTLTISRLE
PEDFAVYYCQQYGSSPWTFGQGTKVEIK (SEQ ID NO: 134)
CDR-L1-nucleotide sequence
CAG AGT ATT GCC GGC AGA TAC (SEQ ID NO: 35)
CDR-L1-amino acid sequence
Q S I A G R Y (SEQ ID NO: 36)
CDR-L2-nucleotide sequence
GGT GCA TCC
CDR-L2-amino acid sequence
G A S
CDR-L3-nucleotide sequence
CAG CAA TAT GGT AGC TCA CCT TGG ACG (SEQ ID NO: 39)
CDR-L3-amino acid sequence
Q Q Y G S S P W T (SEQ ID NO: 40)
Heavy chain (HC)-nucleotide sequence
GAAGTGCAACTGGTGGAGTCTGGGGGAGGCCTGGTCAAGCCTGGGGGGTCCCTGAGACTCTCCTGTGCAGCCTCTGGATT
CATCTTCAGTAGATATAGCATGAACTGGGTCCGCCAGGCTCCAGGGAAGGGGCTGGAGTGGGTCTCATCCATGAGTAGTA
ATAGTAAAAACACATACTACGCAGACTCAGTGAAGGGCCGATTCACCATCTCCAGAGACAACGCCAAAAACTCACTGTTT
CTGCAAATGAACACCCTGAGAGCCGAGGACACGGCTGTTTATTACTGTGCGAGAGATGGATACACCCTCAGGGCTTTTGA
TATCTGGGGCCAAGGGACAATGGTCACCGTCTCTTCAGCCTCCACCAAGGGCCCATCGGTCTTCCCCCTGGCGCCCTGCT
CCAGGAGCACCTCCGAGAGCACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCCGTGAT
GCATGAGGCTCTGCACAACCACTACACACAGAAGTCCCTCTCCCTGTCTCTGGGTAAATGA (SEQ ID NO: 41)
Heavy chain-amino acid sequence
EVQLVESGGGLVKPGGSLRLSCAASGFIFSRYSMNWVRQAPGKGLEWVSSMSSNSKNTYYADSVKGRFTISRDNAKNSLF
LQMNTLRAEDTAVYYCARDGYTLRAFDIWGQGTMVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID NO: 42)
Light chain (LC)-nucleotide sequence
TTACTTCAGGGATCTGGTGAAATTGTGTTGACGCAGTCTCCAGGCACCCTGTCTCTGTCTCCAGGGGAAAGAGACACCCT
CTCCTGCAGGGCCAGTCAGAGTATTGCCGGCAGATACGTAGCCTGGTACCAGCAGAAACCTGGCCAGGCACCCAGACTCC
TCATCTACGGTGCATCCAGCAGGGCCACTGGCATCCCAGACAGATTCAGTGGCAGTGGGTCTGGGACAGACTTCACTCTC
ACCATCAGCAGACTGGAGCCTGAAGATTTTGCAGTGTATTACTGTCAGCAATATGGTAGCTCACCTTGGACGTTCGGCCA
AGGGACCAAGGTGGAAATCAAACGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG (SEQ ID NO: 43)
Light chain (LC)-amino acid sequence
LLQGSGEIVLTQSPGTLSLSPGERDTLSCRASQSIAGRYVAWYQQKPGQAPRLLIYGASSRATGIPDRFSGSGSGTDFTL
TISRLEPEDFAVYYCQQYGSSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC (SEQ ID NO: 44)
REGN7990
Heavy chain variable region (HCVR; V_{H})-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA (SEQ ID NO: 45)
Heavy chain variable region (HCVR; V_{H})-amino acid sequence
EVWLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS (SEQ ID NO: 46)
CDR-H1-nucleotide sequence
GGA TTC ACC TTT AGC AGC TAT GCC (SEQ ID NO: 47)
CDR-H1-amino acid sequence
G F T F S S Y A (SEQ ID NO: 48)
CDR-H2-nucleotide sequence
ATT AGC GGT AGT GGT GGC AGC ACA (SEQ ID NO: 49)
CDR-H2-amino acid sequence
I S G S G G S T (SEQ ID NO: 50)
CDR-H3-nucleotide sequence
GCG AAA GGC CTT ATA GCA CCT CGT CCG ATG GGC TTT GAC TAC (SEQ ID NO: 51)
CDR-H3-amino acid sequence
A K G L I A P R P M G F D Y (SEQ ID NO: 52)
Light chain variable region (LCVR; V_{L})-nucleotide sequence
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGCAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A (SEQ ID NO: 53)
Light chain variable region (LCVR; V_{L})-amino acid sequence
DIQMTQSPSSVSASVGDRVTITCRASQGINSWLAWYQQKPGKAPKLLIYAASSLQSGVPSRFSGSGSGTDFTLTISSLQP
EDFATYYCHQADSFPYTFGQGTKLEIK (SEQ ID NO: 54)
CDR-L1-nucleotide sequence
CAG GGT ATT AAC AGC TGG (SEQ ID NO: 55)
CDR-L1-amino acid sequence
Q G I N S W (SEQ ID NO: 56)
CDR-L2-nucleotide sequence
GCT GCA TCC
CDR-L2-amino acid sequence
A A S
CDR-L3-nucleotide sequence
CAC CAG GCT GAC AGT TTC CCG TAC ACT (SEQ ID NO: 59)
CDR-L3-amino acid sequence
H Q A D S F P Y T (SEQ ID NO: 60)
Heavy chain-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAATGA (SEQ ID NO:
61)
Heavy chain-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK (SEQ ID NO: 62)
Light chain-nucleotide sequence
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGCAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 63)
Light chain-amino acid sequence
LLQGSGDIQMTQSPSSVSASVGDRVTITCRASQGINSWLAWYQQKPGKAPKLLIYAASSLQSGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 64)
REGN15869
Heavy chain variable region (HCVR; V_{H})-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA (SEQ ID NO: 65)
Heavy chain variable region (HCVR; V_{H})-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS (SEQ ID NO: 66)
CDR-H1-nucleotide sequence
GGA TTC ACC TTT AGC AGC TAT GCC (SEQ ID NO: 67)
CDR-H1-amino acid sequence
G F T F S S Y A (SEQ ID NO: 68)
CDR-H2-nucleotide sequence
ATT AGC GGT AGT GGT GGC AGC ACA (SEQ ID NO: 69)
CDR-H2-amino acid sequence
I S G S G G S T (SEQ ID NO: 70)
CDR-H3-nucleotide sequence
GCG AAA GGC CTT ATA GCA CCT CGT CCG ATG GGC TTT GAC TAC (SEQ ID NO: 71)
CDR-H3-amino acid sequence
A K G L I A P R P M G F D Y (SEQ ID NO: 72)
Light chain variable region (LCVR; V_{L})-nucleotide sequence
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGGAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A (SEQ ID NO: 73)
Light chain variable region (LCVR; V_{L})-amino acid sequence
DIQMTQSPSSVSASVGDRVTITCRASQGINSWLAWYQQKPGKAPKLLIYAASSLESGVPSRFSGSGSGTDFTLTISSLQP
EDFATYYCHQADSFPYTFGQGTKLEIK (SEQ ID NO: 74)
CDR-L1-nucleotide sequence
CAG GGT ATT AAC AGC TGG (SEQ ID NO: 75)
CDR-L1-amino acid sequence
Q G I N S W (SEQ ID NO: 76)
CDR-L2-nucleotide sequence
GCT GCA TCC
CDR-L2-amino acid sequence
A A S
CDR-L3-nucleotide sequence
CAC CAG GCT GAC AGT TTC CCG TAC ACT (SEQ ID NO: 79)
CDR-L3-amino acid sequence
H Q A D S F P Y T (SEQ ID NO: 80)
Heavy chain-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAATGA (SEQ ID NO:
81)
Heavy chain-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK (SEQ ID NO: 82)
Light chain-nucleotide sequence
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGGAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 83)
Light chain-amino acid sequence
LLQGSGDIQMTQSPSSVSASVGDRVTITCRASQGINSWLAWYQQKPGKAPKLLIYAASSLESGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 84)
REGN18121
Heavy chain variable region (HCVR; V_{H})-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA (SEQ ID NO: 65)
Heavy chain variable region (HCVR; V_{H})-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS (SEQ ID NO: 66)
CDR-H1-nucleotide sequence
GGA TTC ACC TTT AGC AGC TAT GCC (SEQ ID NO: 67)
CDR-H1-amino acid sequence
G F T F S S Y A (SEQ ID NO: 68)
CDR-H2-nucleotide sequence
ATT AGC GGT AGT GGT GGC AGC ACA (SEQ ID NO: 69)
CDR-H2-amino acid sequence
I S G S G G S T (SEQ ID NO: 70)
CDR-H3-nucleotide sequence
GCG AAA GGC CTT ATA GCA CCT CGT CCG ATG GGC TTT GAC TAC (SEQ ID NO: 71)
CDR-H3-amino acid sequence
A K G L I A P R P M G F D Y (SEQ ID NO: 72)
Light chain variable region (LCVR; V_{L})-nucleotide sequence
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGGAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A (SEQ ID NO: 73)
Light chain variable region (LCVR; V_{L})-amino acid sequence
DIQMTQSPSSVSASVGDRVTITCRASQGINSWLAWYQQKPGKAPKLLIYAASSLESGVPSRFSGSGSGTDFTLTISSLQP
EDFATYYCHQADSFPYTFGQGTKLEIK (SEQ ID NO: 74)
CDR-L1-nucleotide sequence
CAG GGT ATT AAC AGC TGG (SEQ ID NO: 75)
CDR-L1-amino acid sequence
Q G I N S W (SEQ ID NO: 76)
CDR-L2-nucleotide sequence
GCT GCA TCC
CDR-L2-amino acid sequence
A A S
CDR-L3-nucleotide sequence
CAC CAG GCT GAC AGT TTC CCG TAC ACT (SEQ ID NO: 79)
CDR-L3-amino acid sequence
H Q A DS F P Y T (SEQ ID NO: 80)
Heavy chain-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAA (SEQ ID NO: 415)
Heavy chain-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLG (SEQ ID NO: 414)
Light chain-nucleotide sequence
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGGAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 83)
Light chain-amino acid sequence
LLQGSGDIQMTQSPSSVSASVGDRVTITCRASQGINSWLAWYQQKPGKAPKLLIYAASSLESGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 84)
REGN18123
Heavy chain variable region (HCVR; V_{H})-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA (SEQ ID NO: 65)
Heavy chain variable region (HCVR; V_{H})-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS (SEQ ID NO: 66)
CDR-H1-nucleotide sequence
GGA TTC ACC TTT AGC AGC TAT GCC (SEQ ID NO: 67)
CDR-H1-amino acid sequence
G F T F S S Y A (SEQ ID NO: 68)
CDR-H2-nucleotide sequence
ATT AGC GGT AGT GGT GGC AGC ACA (SEQ ID NO: 69)
CDR-H2-amino acid sequence
I S G S G G S T (SEQ ID NO: 70)
CDR-H3-nucleotide sequence
GCG AAA GGC CTT ATA GCA CCT CGT CCG ATG GGC TTT GAC TAC (SEQ ID NO: 71)
CDR-H3-amino acid sequence
A K G L I A P R P M G F D Y (SEQ ID NO: 72)
Light chain variable region (LCVR; V_{L})-nucleotide sequence
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGGAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A (SEQ ID NO: 73)
Light chain variable region (LCVR; V_{L})-amino acid sequence
DIQMTQSPSSVSASVGDRVTITCRASQGINSWLAWYQQKPGKAPKLLIYAASSLESGVPSRFSGSGSGTDFTLTISSLQP
EDFATYYCHQADSFPYTFGQGTKLEIK (SEQ ID NO: 74)
CDR-L1-nucleotide sequence
CAG GGT ATT AAC AGC TGG (SEQ ID NO: 75)
CDR-L1-amino acid sequence
Q G I N S W (SEQ ID NO: 76)
CDR-L2-nucleotide sequence
GCT GCA TCC
CDR-L2-amino acid sequence
A A S
CDR-L3-nucleotide sequence
CAC CAG GCT GAC AGT TTC CCG TAC ACT (SEQ ID NO: 79)
CDR-L3-amino acid sequence
H Q A D S F P Y T (SEQ ID NO: 80)
Heavy chain-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGT (SEQ ID NO: 417)
Heavy chain-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSL (SEQ ID NO: 416)
Light chain-nucleotide sequence
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGGAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 83)
Light chain-amino acid sequence
LLQGSGDIQMTQSPSSVSASVGDRVTITCRASQGINSWLAWYQQKPGKAPKLLIYAASSLESGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 84)
REGN8070
Heavy chain variable region (HCVR, V_{H})-nucleotide sequence
CAGGTGCAGCTGGTGGAGTCTGGGGGAGGCGTGGGCCAGCCTGGGAGGTCCCTGAGACTGTCCTGTGCAGCCTCTGGATT
CACCTTCAGCAGGAATGCCATGCACTGGGTCCGCCAGGCTCCAGGCAAGGGGCTGGAGTGGGTGGCAGTCATATCATATG
ATGGAAGTAATAAACACTATGCAGACTCCGTGAAGGGCCGATTCACCATCTCCAGAGACAATTCCAAGAACACGCTGTAT
CTGGAAATGAACAGCCTGAGAGTTGAGGACACGGCTGTGTATTATTGTGCGAAAGGGGGGATTCCTTTTGACTACTGGGG
CCAGGGAACCCTGGTCACCGTCTCCTCA (SEQ ID NO: 85)
Heavy chain variable region (HCVR, V_{H})-amino acid sequence
QVQLVESGGGVGQPGRSLRLSCAASGFTFSRNAMHWVRQAPGKGLEWVAVISYDGSNKHYADSVKGRFTISRDNSKNTLY
LEMNSLRVEDTAVYYCAKGGIPFDYWGQGTLVTVSS (SEQ ID NO: 86)
CDR-H1-nucleotide sequence
GGA TTC ACC TTC AGC AGG AAT GCC (SEQ ID NO: 87)
CDR-H1-amino acid sequence
G F T F S R N A (SEQ ID NO: 88)
CDR-H2-nucleotide sequence
ATA TCA TAT GAT GGA AGT AAT AAA (SEQ ID NO: 89)
CDR-H2-amino acid sequence
I S Y D G S N K (SEQ ID NO: 90)
CDR-H3-nucleotide sequence
GCG AAA GGG GGG ATT CCT TTT GAC TAC (SEQ ID NO: 91)
CDR-H3-amino acid sequence
A K G G I P F D Y (SEQ ID NO: 92)
Light chain variable region (LCVR, V_{L})-nucleotide sequence
GATATTGTGATGACCCAGTCTCCACTCTCCTCACCTGTCACCCTTGGACAGCCGGCCTCCATCTCCTGCAGGTCTAGTCA
AAGCCTCGTACACTTTGATGGAAACACCTACTTGAGTTGGCTTCACCAGAGGCCAGGCCAGCCTCCAAGACTCCTAATTT
ATAAGATTTCTAACCGCTTCTCTGGGGTCCCAGACAGATTCAGTGGCAGTGGGGCAGGGACAGATTTCACACTGAAAATC
AGCAGGGTGGAACCTGAAGATGTCGGGGTTTATTACTGCATGCATGCTACACAATTTCCGTACACTTTTGGCCAGGGGAC
CAAGCTGGAGATCAAA (SEQ ID NO: 93)
Light chain variable region (LCVR, V_{L})-amino acid sequence
DIVMTQSPLSSPVTLGQPASISCRSSQSLVHFDGNTYLSWLHQRPGQPPRLLIYKISNRFSGVPDRFSGSGAGTDFTLKI
SRVEPEDVGVYYCMHATQFPYTFGQGTKLEIK (SEQ ID NO: 94)
CDR-L1-nucleotide sequence
CAA AGC CTC GTA CAC TTT GAT GGA AAC ACC TAC (SEQ ID NO: 95)
CDR-L1-amino acid sequence
Q S L V H F D G N T Y (SEQ ID NO: 96)
CDR-L2-nucleotide sequence
AAG ATT TCT
CDR-L2-amino acid sequence
K I S
CDR-L3-nucleotide sequence
ATG CAT GCT ACA CAA TTT CCG TAC ACT (SEQ ID NO: 99)
CDR-L3-amino acid sequence
M H A T Q F P Y T (SEQ ID NO: 100)
Heavy chain (HC)-nucleotide sequence
CAGGTGCAGCTGGTGGAGTCTGGGGGAGGCGTGGGCCAGCCTGGGAGGTCCCTGAGACTGTCCTGTGCAGCCTCTGGATT
CACCTTCAGCAGGAATGCCATGCACTGGGTCCGCCAGGCTCCAGGCAAGGGGCTGGAGTGGGTGGCAGTCATATCATATG
ATGGAAGTAATAAACACTATGCAGACTCCGTGAAGGGCCGATTCACCATCTCCAGAGACAATTCCAAGAACACGCTGTAT
CTGGAAATGAACAGCCTGAGAGTTGAGGACACGGCTGTGTATTATTGTGCGAAAGGGGGGATTCCTTTTGACTACTGGGG
CCAGGGAACCCTGGTCACCGTCTCCTCAGCCTCCACCAAGGGCCCATCGGTCTTCCCCCTGGCGCCCTGCTCCAGGAGCA
CCTCCGAGAGCACAGCCGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGCAGCGTGGTGACCGT
GCCCTCCAGCAGCTTGGGCACGAAGACCTACACCTGCAACGTAGATCACAAGCCCAGCAACACCAAGGTGGACAAGAGAG
TTGAGTCCAAATATGGTCCCCCATGCCCACCCTGCCCAGCACCTGAGTTCCTGGGGGGACCATCAGTCTTCCTGTTCCCC
CCAAAACCCAAGGACACTCTCATGATCTCCCGGACCCCTGAGGTCACGTGCGTGGTGGTGGACGTGAGCCAGGAAGACCC
CGAGGTCCAGTTCAACTGGTACGTGGATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCCCCGAGAGCCACAGGTGTACACCCT
GCCCCCATCCCAGGAGGAGATGACCAAGAACCAGGTCAGCCTGACCTGCCTGGTCAAAGGCTTCTACCCCAGCGACATCG
CCGTGGAGTGGGAGAGCAATGGGCAGCCGGAGAACAACTACAAGACCACGCCTCCCGTGCTGGACTCCGACGGCTCCTTC
TTCCTCTACAGCAGGCTCACCGTGGACAAGAGCAGGTGGCAGGAGGGGAATGTCTTCTCATGCTCCGTGATGCATGAGGC
TCTGCACAACCACTACACACAGAAGTCCCTCTCCCTGTCTCTGGGTAAATGA (SEQ ID NO: 101)
Heavy chain-amino acid sequence
QVQLVESGGGVGQPGRSLRLSCAASGFTFSRNAMHWVRQAPGKGLEWVAVISYDGSNKHYADSVKGRFTISRDNSKNTLY
LEMNSLRVEDTAVYYCAKGGIPFDYWGQGTLVTVSSASTKGPSVFPLAPCSRSTSESTAALGCLVKDYFPEPVTVSWNSG
ALTSGVHTFPAVLQSSGLYSLSSVVTVPSSSLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK (SEQ ID NO: 102)
Light chain (LC)-nucleotide sequence
CTGCTGCAAGGCTCTGGCGATATTGTGATGACCCAGTCTCCACTCTCCTCACCTGTCACCCTTGGACAGCCGGCCTCCAT
CTCCTGCAGGTCTAGTCAAAGCCTCGTACACTTTGATGGAAACACCTACTTGAGTTGGCTTCACCAGAGGCCAGGCCAGC
CTCCAAGACTCCTAATTTATAAGATTTCTAACCGCTTCTCTGGGGTCCCAGACAGATTCAGTGGCAGTGGGGCAGGGACA
GATTTCACACTGAAAATCAGCAGGGTGGAACCTGAAGATGTCGGGGTTTATTACTGCATGCATGCTACACAATTTCCGTA
CACTTTTGGCCAGGGGACCAAGCTGGAGATCAAACGAACTGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCTCAG
CAGCACCCTGACGCTGAGCAAAGCAGACTACGAGAAACACAAAGTCTACGCCTGCGAAGTCACCCATCAGGGCCTGAGCT
CGCCCGTCACAAAGAGCTTCAACAGGGGAGAGTGTTAG (SEQ ID NO: 103)
Light chain (LC)-amino acid sequence
LLQGSGDIVMTQSPLSSPVTLGQPASISCRSSQSLVHFDGNTYLSWLHQRPGQPPRLLIYKISNRFSGVPDRFSGSGAGT
DFTLKISRVEPEDVGVYYCMHATQFPYTFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC (SEQ ID NO:
104)
REGN8072
Heavy chain variable region (HCVR, V_{H})-nucleotide sequence
CAGGTGCAGCTGGTGGAGTCTGGGGGAGGCGTGGTCCAGCCTGCGAGGTCCCTGAGACTCTCCTGTGCAGCCTCTGGATT
CGCCTTCAGTAGGTCTGCCATGCACTGGGTCCGCCAGGCTCCAGGCAAGGGGCTGGAGTGGGTGGCAGTTATATCATATG
ATGGAAGTAATAAATACTATACAGACTCCGTGAAGGGCCGATTCACCATCTCCAGAGACAATTCCAAGAACACGCTGTAT
CTGCAAATGAACACCCTGAGAGCTGAGGACACGGCTCTTTATTACTGTGCGAAAATGTATACAACTATGGACTCTTTTGA
CTACTGGGGCCAGGGAACCCTGGTCACCGTCTCCTCA (SEQ ID NO: 105)
Heavy chain variable region (HCVR, V_{H})-amino acid sequence
QVQLVESGGGVVQPARSLRLSCAASGFAFSRSAMHWVRQAPGKGLEWVAVISYDGSNKYYTDSVKGRFTISRDNSKNTLY
LQMNTLRAEDTALYYCAKMYTTMDSFDYWGQGTLVTVSS (SEQ ID NO: 106)
CDR-H1-nucleotide sequence
GGA TTC GCC TTC AGT AGG TCT GCC (SEQ ID NO: 107)
CDR-H1-amino acid sequence
G F A F S R S A (SEQ ID NO: 108)
CDR-H2-nucleotide sequence
ATA TCA TAT GAT GGA AGT AAT AAA (SEQ ID NO: 109)
CDR-H2-amino acid sequence
I S Y D G S N K (SEQ ID NO: 110)
CDR-H3-nucleotide sequence
GCG AAA ATG TAT ACA ACT ATG GAC TCT TTT GAC TAC (SEQ ID NO: 111)
CDR-H3-amino acid sequence
A K M Y T T M D S F D Y (SEQ ID NO: 112)
Light chain variable region (LCVR, V_{L})-nucleotide sequence
GACATCCAGTTGACCCAGTCTCCATCCTTCCTGTCTGCATCTGTAGGAGACAGAGTCACCATCACTTGCTGGGCCAGTCA
GGGCATTAGCAGTTATTTAGCCTGGTATCAGCAAAAACCAGGGAAAGCCCCTAAGCTCCTGATCTATGCTGCATCCACTT
TGCAAAGTGGGGTCCCATCAAGGTTCAGCGGCAGTGGATCTGGGACAGAATTCACTCTCACAATCAGCAGCCTGCAGCCT
GAAGATTTTGCACTTTATTACTGTCAACAGCTTAATAGTTACCCTCGGACGTTCGGCCAAGGGACCAAGGTGGAAATCAA
A (SEQ ID NO: 113)
Light chain variable region (LCVR, V_{L})-amino acid sequence
DIQLTQSPSFLSASVGDRVTITCWASQGISSYLAWYQQKPGKAPKLLIYAASTLQSGVPSRFSGSGSGTEFTLTISSLQP
EDFALYYCQQLNSYPRTFGQGTKVEIK (SEQ ID NO: 114)
CDR-L1-nucleotide sequence
CAG GGC ATT AGC AGT TAT (SEQ ID NO: 115)
CDR-L1-amino acid sequence
Q G I S SY (SEQ ID NO: 116)
CDR-L2-nucleotide sequence
GCT GCA TCC
CDR-L2-amino acid sequence
A A S
CDR-L3-nucleotide sequence
CAA CAG CTT AAT AGT TAC CCT CGG ACG (SEQ ID NO: 119)
CDR-L3-amino acid sequence
Q Q L N S Y P R T (SEQ ID NO: 120)
Heavy chain (HC)-nucleotide sequence
CAGGTGCAGCTGGTGGAGTCTGGGGGAGGCGTGGTCCAGCCTGCGAGGTCCCTGAGACTCTCCTGTGCAGCCTCTGGATT
CGCCTTCAGTAGGTCTGCCATGCACTGGGTCCGCCAGGCTCCAGGCAAGGGGCTGGAGTGGGTGGCAGTTATATCATATG
ATGGAAGTAATAAATACTATACAGACTCCGTGAAGGGCCGATTCACCATCTCCAGAGACAATTCCAAGAACACGCTGTAT
CTGCAAATGAACACCCTGAGAGCTGAGGACACGGCTCTTTATTACTGTGCGAAAATGTATACAACTATGGACTCTTTTGA
CTACTGGGGCCAGGGAACCCTGGTCACCGTCTCCTCAGCCTCCACCAAGGGCCCATCGGTCTTCCCCCTGGCGCCCTGCT
CCAGGAGCACCTCCGAGAGCACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCCGTGAT
GCATGAGGCTCTGCACAACCACTACACACAGAAGTCCCTCTCCCTGTCTCTGGGTAAATGA (SEQ ID NO: 121)
Heavy chain-amino acid sequence
QVQLVESGGGVVQPARSLRLSCAASGFAFSRSAMHWVRQAPGKGLEWVAVISYDGSNKYYTDSVKGRFTISRDNSKNTLY
LQMNTLRAEDTALYYCAKMYTTMDSFDYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID NO: 122)
Light chain (LC)-nucleotide sequence
CTGCTGCAAGGCTCTGGCGACATCCAGTTGACCCAGTCTCCATCCTTCCTGTCTGCATCTGTAGGAGACAGAGTCACCAT
CACTTGCTGGGCCAGTCAGGGCATTAGCAGTTATTTAGCCTGGTATCAGCAAAAACCAGGGAAAGCCCCTAAGCTCCTGA
TCTATGCTGCATCCACTTTGCAAAGTGGGGTCCCATCAAGGTTCAGCGGCAGTGGATCTGGGACAGAATTCACTCTCACA
ATCAGCAGCCTGCAGCCTGAAGATTTTGCACTTTATTACTGTCAACAGCTTAATAGTTACCCTCGGACGTTCGGCCAAGG
GACCAAGGTGGAAATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 123)
Light chain (LC)-amino acid sequence
LLQGSGDIQLTQSPSFLSASVGDRVTITCWASQGISSYLAWYQQKPGKAPKLLIYAASTLQSGVPSRFSGSGSGTEFTLT
ISSLQPEDFALYYCQQLNSYPRTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 124)
REGN9267
Heavy chain variable region (HCVR, V_{H})-nucleotide sequence
GAAGTGCAACTGGTGGAGTCTGGGGGAGGCCTGGTCAAGCCTGGGGGGTCCCTGAGACTCTCCTGTGCAGCCTCTGGATT
CATCTTCAGTAGATATAGCATGAACTGGGTCCGCCAGGCTCCAGGGAAGGGGCTGGAGTGGGTCTCATCCATGAGTAGTA
ATAGTAAAAACACATACTACGCAGACTCAGTGAAGGGCCGATTCACCATCTCCAGAGACAACGCCAAAAACTCACTGTTT
CTGCAAATGAACACCCTGAGAGCCGAGGACACGGCTGTTTATTACTGTGCGAGAGATGGATACACCCTCAGGGCTTTTGA
TATCTGGGGCCAAGGGACAATGGTCACCGTCTCTTCA (SEQ ID NO: 125)
Heavy chain variable region (HCVR, V_{H})-amino acid sequence
EVQLVESGGGLVKPGGSLRLSCAASGFIFSRYSMNWVRQAPGKGLEWVSSMSSNSKNTYYADSVKGRFTISRDNAKNSLF
LQMNTLRAEDTAVYYCARDGYTLRAFDIWGQGTMVTVSS (SEQ ID NO: 126)
CDR-H1-nucleotide sequence
GGA TTC ATC TTC AGT AGA TAT AGC (SEQ ID NO: 127)
CDR-H1-amino acid sequence
G F I F S R Y S (SEQ ID NO: 128)
CDR-H2-nucleotide sequence
ATG AGT AGT AAT AGT AAA AAC ACA (SEQ ID NO: 129)
CDR-H2-amino acid sequence
M S S N S K N T (SEQ ID NO: 130)
CDR-H3-nucleotide sequence
GCG AGA GAT GGA TAC ACC CTC AGG GCT TTT GAT ATC (SEQ ID NO: 131)
CDR-H3-amino acid sequence
A R D G Y T L R A F D I (SEQ ID NO: 132)
Light chain variable region (LCVR, V_{L})-nucleotide sequence
GAAATTGTGTTGACGCAGTCTCCAGGCACCCTGTCTCTGTCTCCAGGGGAAAGAGACACCCTCTCCTGCAGGGCCAGTCA
GAGTATTGCCGGCAGATACGTAGCCTGGTACCAGCAGAAACCTGGCCAGGCACCCAGACTCCTCATCTACGGTGCATCCA
GCAGGGCCACTGGCATCCCAGACAGATTCAGTGGCAGTGGGTCTGGGACAGACTTCACTCTCACCATCAGCAGACTGGAG
CCTGAAGATTTTGCAGTGTATTACTGTCAGCAATATGGTAGCTCACCTTGGACGTTCGGCCAAGGGACCAAGGTGGAAAT
CAAA (SEQ ID NO: 133)
Light chain variable region (LCVR, V_{L})-amino acid sequence
EIVLTQSPGTLSLSPGERDTLSCRASQSIAGRYVAWYQQKPGQAPRLLIYGASSRATGIPDRFSGSGSGTDFTLTISRLE
PEDFAVYYCQQYGSSPWTFGQGTKVEIK (SEQ ID NO: 134)
CDR-L1-nucleotide sequence
CAG AGT ATT GCC GGC AGA TAC (SEQ ID NO: 135)
CDR-L1-amino acid sequence
Q S I A G R Y (SEQ ID NO: 136)
CDR-L2-nucleotide sequence
GGT GCA TCC
CDR-L2-amino acid sequence
G A S
CDR-L3-nucleotide sequence
CAG CAA TAT GGT AGC TCA CCT TGG ACG (SEQ ID NO: 139)
CDR-L3-amino acid sequence
Q Q Y G S S P W T (SEQ ID NO: 140)
Heavy chain (HC)-nucleotide sequence
TTACTTCAGGGATCTGGTGAAGTGCAACTGGTGGAGTCTGGGGGAGGCCTGGTCAAGCCTGGGGGGTCCCTGAGACTCTC
CTGTGCAGCCTCTGGATTCATCTTCAGTAGATATAGCATGAACTGGGTCCGCCAGGCTCCAGGGAAGGGGCTGGAGTGGG
TCTCATCCATGAGTAGTAATAGTAAAAACACATACTACGCAGACTCAGTGAAGGGCCGATTCACCATCTCCAGAGACAAC
GCCAAAAACTCACTGTTTCTGCAAATGAACACCCTGAGAGCCGAGGACACGGCTGTTTATTACTGTGCGAGAGATGGATA
CACCCTCAGGGCTTTTGATATCTGGGGCCAAGGGACAATGGTCACCGTCTCTTCAGCCTCCACCAAGGGCCCATCGGTCT
TCCCCCTGGCGCCCTGCTCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCACCCTGCCCAGCACCTGAGTTCCTG
GGGGGACCATCAGTCTTCCTGTTCCCCCCAAAACCCAAGGACACTCTCATGATCTCCCGGACCCCTGAGGTCACGTGCGT
GGTGGTGGACGTGAGCCAGGAAGACCCCGAGGTCCAGTTCAACTGGTACGTGGATGGCGTGGAGGTGCATAATGCCAAGA
CAAAGCCGCGGGAGGAGCAGTTCAACAGCACGTACCGTGTGGTCAGCGTCCTCACCGTCCTGCACCAGGACTGGCTGAAC
GGCAAGGAGTACAAGTGCAAGGTCTCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGAACCAGGTCAGCCTGACCTGCCTGG
TCAAAGGCTTCTACCCCAGCGACATCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTCACCGTGGACAAGAGCAGGTGGCAGGAGGGGAATGT
CTTCTCATGCTCCGTGATGCATGAGGCTCTGCACAACCACTACACACAGAAGTCCCTCTCCCTGTCTCTGGGTAAATGA
(SEQ ID NO: 141)
Heavy chain-amino acid sequence
LLQGSGEVQLVESGGGLVKPGGSLRLSCAASGFIFSRYSMNWVRQAPGKGLEWVSSMSSNSKNTYYADSVKGRFTISRDN
AKNSLFLQMNTLRAEDTAVYYCARDGYTLRAFDIWGQGTMVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK (SEQ ID NO: 142)
Light chain (LC)-nucleotide sequence
GAAATTGTGTTGACGCAGTCTCCAGGCACCCTGTCTCTGTCTCCAGGGGAAAGAGACACCCTCTCCTGCAGGGCCAGTCA
GAGTATTGCCGGCAGATACGTAGCCTGGTACCAGCAGAAACCTGGCCAGGCACCCAGACTCCTCATCTACGGTGCATCCA
GCAGGGCCACTGGCATCCCAGACAGATTCAGTGGCAGTGGGTCTGGGACAGACTTCACTCTCACCATCAGCAGACTGGAG
CCTGAAGATTTTGCAGTGTATTACTGTCAGCAATATGGTAGCTCACCTTGGACGTTCGGCCAAGGGACCAAGGTGGAAAT
CAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG (SEQ ID NO: 143)
Light chain (LC)-amino acid sequence
EIVLTQSPGTLSLSPGERDTLSCRASQSIAGRYVAWYQQKPGQAPRLLIYGASSRATGIPDRFSGSGSGTDFTLTISRLE
PEDFAVYYCQQYGSSPWTFGQGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC (SEQ ID NO: 144)
REGN7988
Heavy chain variable region (HCVR, V_{H})-nucleotide sequence
CAGGTGCAGCTGGTGGAGTCTGGGGGAGGCGTGGTCCAGCCTGGGAGGTCCCTGAGACTCTCCTGTGCAGCGTCTGGATT
CACCTTCAGTGGCTATGGCATACACTGGGTCCGCCAGGCTCCAGGCAAGGGACTGGTGTGGGTGGCAGTTATATGGTATG
ATGGAAGTTTTAAATACTATGCAGACTCCGTGAAGGGCCGATTCACCATCTCCAGAGACAATTCCAAGAACACGCTGTAT
CTGCAGATGAACAGCCTGAGAGCCGAGGACACGGCTGTGTATTACTGTGCGAGAGGTGATAGCAGCTCGTCCGGACGGTA
CTACTACTACGGTATGGACGTCTGGGGCCAAGGGACCACGGTCACCGTCTCCTCA (SEQ ID NO: 145)
Heavy chain variable region (HCVR, V_{H})-amino acid sequence
QVQLVESGGGVVQPGRSLRLSCAASGFTFSGYGIHWVRQAPGKGLVWVAVIWYDGSFKYYADSVKGRFTISRDNSKNTLY
LQMNSLRAEDTAVYYCARGDSSSSGRYYYYGMDVWGQGTTVTVSS (SEQ ID NO: 146)
CDR-H1-nucleotide sequence
GGA TTC ACC TTC AGT GGC TAT GGC (SEQ ID NO: 147)
CDR-H1-amino acid sequence
G F T F S G Y G (SEQ ID NO: 148)
CDR-H2-nucleotide sequence
ATA TGG TAT GAT GGA AGT TTT AAA (SEQ ID NO: 149)
CDR-H2-amino acid sequence
I W Y D G S F K (SEQ ID NO: 150)
CDR-H3-nucleotide sequence
GCG AGA GGT GAT AGC AGC TCG TCC GGA CGG TAC TAC TAC TAC GGT ATG GAC GTC (SEQ ID NO:
151)
CDR-H3-amino acid sequence
A R G D S S S S G R Y Y Y Y G M D V (SEQ ID NO:
152)
Light chain variable region (LCVR, V_{L})-nucleotide sequence
GACATCCAGATGACCCAGTCTCCATCCTCCCTGTCTGCATCTGTAGGAGACAGAGTCACCATCACTTGCCGGGCAAGTCA
GGGCATTAGAAATGATTTAGGCTGGTATCAGCAGAAACCAGGGACAGCCCCTAAGCGCCTGATCTTTGCTGCATCCAGTT
TGCAAAGTGGGGTCCCATCAAGGTTCAGCGGCAGTGGATCTGGGACAGAATTCACTCTCACAATCAGCAGCCTGCAGCCT
GAAGATTTTGCGACTTATTACTGTCTACAGCATAATAATTACCCTCCCACTTTCGGCGGAGGGACCAAGGTGGAGATCAA
A (SEQ ID NO: 153)
Light chain variable region (LCVR, V_{L})-amino acid sequence
DIQMTQSPSSLSASVGDRVTITCRASQGIRNDLGWYQQKPGTAPKRLIFAASSLQSGVPSRFSGSGSGTEFTLTISSLQP
EDFATYYCLQHNNYPPTFGGGTKVEIK (SEQ ID NO: 154)
CDR-L1-nucleotide sequence
CAG GGC ATT AGA AAT GAT (SEQ ID NO: 155)
CDR-L1-amino acid sequence
Q G I R N D (SEQ ID NO: 156)
CDR-L2-nucleotide sequence
GCT GCA TCC
CDR-L2-amino acid sequence
A A S
CDR-L3-nucleotide sequence
CTA CAG CAT AAT AAT TAC CCT CCC ACT (SEQ ID NO: 159)
CDR-L3-amino acid sequence
L Q H N N Y P P T (SEQ ID NO: 160)
Heavy chain (HC)-nucleotide sequence
CAGGTGCAGCTGGTGGAGTCTGGGGGAGGCGTGGTCCAGCCTGGGAGGTCCCTGAGACTCTCCTGTGCAGCGTCTGGATT
CACCTTCAGTGGCTATGGCATACACTGGGTCCGCCAGGCTCCAGGCAAGGGACTGGTGTGGGTGGCAGTTATATGGTATG
ATGGAAGTTTTAAATACTATGCAGACTCCGTGAAGGGCCGATTCACCATCTCCAGAGACAATTCCAAGAACACGCTGTAT
CTGCAGATGAACAGCCTGAGAGCCGAGGACACGGCTGTGTATTACTGTGCGAGAGGTGATAGCAGCTCGTCCGGACGGTA
CTACTACTACGGTATGGACGTCTGGGGCCAAGGGACCACGGTCACCGTCTCCTCAGCCTCCACCAAGGGCCCATCGGTCT
TCCCCCTGGCGCCCTGCTCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCACCCTGCCCAGCACCTGAGTTCCTG
GGGGGACCATCAGTCTTCCTGTTCCCCCCAAAACCCAAGGACACTCTCATGATCTCCCGGACCCCTGAGGTCACGTGCGT
GGTGGTGGACGTGAGCCAGGAAGACCCCGAGGTCCAGTTCAACTGGTACGTGGATGGCGTGGAGGTGCATAATGCCAAGA
CAAAGCCGCGGGAGGAGCAGTTCAACAGCACGTACCGTGTGGTCAGCGTCCTCACCGTCCTGCACCAGGACTGGCTGAAC
GGCAAGGAGTACAAGTGCAAGGTCTCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGAACCAGGTCAGCCTGACCTGCCTGG
TCAAAGGCTTCTACCCCAGCGACATCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTCACCGTGGACAAGAGCAGGTGGCAGGAGGGGAATGT
CTTCTCATGCTCCGTGATGCATGAGGCTCTGCACAACCACTACACACAGAAGTCCCTCTCCCTGTCTCTGGGTAAATGA
(SEQ ID NO: 161)
Heavy chain-amino acid sequence
QVQLVESGGGVVQPGRSLRLSCAASGFTFSGYGIHWVRQAPGKGLVWVAVIWYDGSFKYYADSVKGRFTISRDNSKNTLY
LQMNSLRAEDTAVYYCARGDSSSSGRYYYYGMDVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK (SEQ ID NO: 162)
Light chain (LC)-nucleotide sequence
CTGCTGCAAGGCTCTGGCGACATCCAGATGACCCAGTCTCCATCCTCCCTGTCTGCATCTGTAGGAGACAGAGTCACCAT
CACTTGCCGGGCAAGTCAGGGCATTAGAAATGATTTAGGCTGGTATCAGCAGAAACCAGGGACAGCCCCTAAGCGCCTGA
TCTTTGCTGCATCCAGTTTGCAAAGTGGGGTCCCATCAAGGTTCAGCGGCAGTGGATCTGGGACAGAATTCACTCTCACA
ATCAGCAGCCTGCAGCCTGAAGATTTTGCGACTTATTACTGTCTACAGCATAATAATTACCCTCCCACTTTCGGCGGAGG
GACCAAGGTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 163)
Light chain (LC)-amino acid sequence
LLQGSGDIQMTQSPSSLSASVGDRVTITCRASQGIRNDLGWYQQKPGTAPKRLIFAASSLQSGVPSRFSGSGSGTEFTLT
ISSLQPEDFATYYCLQHNNYPPTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 164)
REGN5619
Heavy chain variable region (HCVR, V_{H})-nucleotide sequence
GAGGTGCAGCTGGTGGAGTCTGGGGGAGGCTTGGTCCAGCCTGGGGGGTCCCTGAAACTCTCCTGTGCAGCCTCTGGGTT
CACCTTCAGTGGCTCTGCTATGCACTGGGTCCGCCAGGCTTCCGGGAAAGGGCTGGAGTGGGTTGGCCGTATTACAAGCA
AAGCTAACAGTTACGCGACAGCATATGATGCGTCGGTGAAAGGCAGGTTCACCATCTCCAGAGATGATTCAAAGAACACG
GCGTATCTGCAAATGAACAGCCTGAAAACCGAGGACACGGCCGTGTATTACTGTACTAGGCAACGATTTTTGGAGTTTTT
ATTCCTTGACTACTGGGGCCAGGGAACCCTGGTCACCGTCTCCTCA (SEQ ID NO: 165)
Heavy chain variable region (HCVR, V_{H})-amino acid sequence
EVQLVESGGGLVQPGGSLKLSCAASGFTFSGSAMHWVRQASGKGLEWVGRITSKANSYATAYDASVKGRFTISRDDSKNT
AYLQMNSLKTEDTAVYYCTRQRFLEFLFLDYWGQGTLVTVSS (SEQ ID NO: 166)
CDR-H1-nucleotide sequence
GGG TTC ACC TTC AGT GGC TCT GCT (SEQ ID NO: 167)
CDR-H1-amino acid sequence
G F T F S G S A (SEQ ID NO: 168)
CDR-H2-nucleotide sequence
ATT ACA AGC AAA GCT AAC AGT TAC GCG ACA (SEQ ID NO: 169)
CDR-H2-amino acid sequence
I T S K A N S Y A T (SEQ ID NO: 170)
CDR-H3-nucleotide sequence
ACT AGG CAA CGA TTT TTG GAG TTT TTA TTC CTT GAC TAC (SEQ ID NO: 171)
CDR-H3-amino acid sequence
T R Q R F L E F L F D Y (SEQ ID NO: 172)
Light chain variable region (LCVR, V_{L})-nucleotide sequence
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA (SEQ ID NO: 173)
Light chain variable region (LCVR, V_{L})-amino acid sequence
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK (SEQ ID NO: 174)
CDR-L1-nucleotide sequence
CAG AGC ATT AGC AGC TAT (SEQ ID NO: 175)
CDR-L1-amino acid sequence
Q S I S S Y (SEQ ID NO: 176)
CDR-L2-nucleotide sequence
GCT GCA TCC
CDR-L2-amino acid sequence
A A S
CDR-L3-nucleotide sequence
CAA CAG AGT TAC AGT ACC CCT CCG ATC ACC (SEQ ID NO: 179)
CDR-L3-amino acid sequence
Q Q S Y S T P P I T (SEQ ID NO: 180)
Heavy chain (HC)-nucleotide sequence
GAGGTGCAGCTGGTGGAGTCTGGGGGAGGCTTGGTCCAGCCTGGGGGGTCCCTGAAACTCTCCTGTGCAGCCTCTGGGTT
CACCTTCAGTGGCTCTGCTATGCACTGGGTCCGCCAGGCTTCCGGGAAAGGGCTGGAGTGGGTTGGCCGTATTACAAGCA
AAGCTAACAGTTACGCGACAGCATATGATGCGTCGGTGAAAGGCAGGTTCACCATCTCCAGAGATGATTCAAAGAACACG
GCGTATCTGCAAATGAACAGCCTGAAAACCGAGGACACGGCCGTGTATTACTGTACTAGGCAACGATTTTTGGAGTTTTT
ATTCCTTGACTACTGGGGCCAGGGAACCCTGGTCACCGTCTCCTCAGCCTCCACCAAGGGCCCATCGGTCTTCCCCCTGG
CGCCCTGCTCCAGGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGCACCTGAGTTCCTGGGGGGACCA
TCAGTCTTCCTGTTCCCCCCAAAACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTCTGGGTAAATGA (SEQ ID
NO: 181)
Heavy chain-amino acid sequence
EVQLVESGGGLVQPGGSLKLSCAASGFTFSGSAMHWVRQASGKGLEWVGRITSKANSYATAYDASVKGRFTISRDDSKNT
AYLQMNSLKTEDTAVYYCTRQRFLEFLFLDYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK (SEQ ID NO: 182)
Light chain (LC)-nucleotide sequence
CTGCTGCAAGGCTCTGGCGACATCCAGATGACCCAGTCTCCATCCTCCCTGTCTGCATCTGTAGGAGACAGAGTCACCAT
CACTTGCCGGGCAAGTCAGAGCATTAGCAGCTATTTAAATTGGTATCAGCAGAAACCAGGGAAAGCCCCTAAGCTCCTGA
TCTATGCTGCATCCAGTTTGCAAAGTGGGGTCCCGTCAAGGTTCAGTGGCAGTGGATCTGGGACAGATTTCACTCTCACC
ATCAGCAGTCTGCAACCTGAAGATTTTGCAACTTACTACTGTCAACAGAGTTACAGTACCCCTCCGATCACCTTCGGCCA
AGGGACACGACTGGAGATTAAACGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG (SEQ ID NO: 183)
Light chain (LC)-amino acid sequence
LLQGSGDIQMTQSPSSLSASVGDRVTITCRASQSISSYLNWYQQKPGKAPKLLIYAASSLQSGVPSRFSGSGSGTDFTLT
ISSLQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC (SEQ ID NO: 184)
REGN7989
Heavy chain variable region (HCVR, V_{H})-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA (SEQ ID NO: 186)
Heavy chain variable region (HCVR, V_{H})-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS (SEQ ID NO: 187)
CDR-H1-nucleotide sequence
GGA TTC ACC TTT AGC AGC TAT GCC (SEQ ID NO: 188)
CDR-H1-amino acid sequence
G F T F S S Y A (SEQ ID NO: 189)
CDR-H2-nucleotide sequence
ATT AGC GGT AGT GGT GGC AGC ACA (SEQ ID NO: 190)
CDR-H2-amino acid sequence
I S G S G G S T (SEQ ID NO: 191)
CDR-H3-nucleotide sequence
GCG AAA GGC CTT ATA GCA CCT CGT CCG ATG GGC TTT GAC TAC (SEQ ID NO: 192)
CDR-H3-amino acid sequence
A K G L I A P R P M G F D Y (SEQ ID NO: 193)
Light chain variable region (LCVR, V_{L})-nucleotide sequence
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGCAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A (SEQ ID NO: 194)
Light chain variable region (LCVR, V_{L})-amino acid sequence
DIQMTQSPSSVSASVGDRVTITCRASQGINSWLAWYQQKPGKAPKLLIYAASSLQSGVPSRFSGSGSGTDFTLTISSLQP
EDFATYYCHQADSFPYTFGQGTKLEIK (SEQ ID NO: 195)
CDR-L1-nucleotide sequence
CAG GGT ATT AAC AGC TGG (SEQ ID NO: 196)
CDR-L1-amino acid sequence
Q G I N S W (SEQ ID NO: 197)
CDR-L2-nucleotide sequence
GCT GCA TCC
CDR-L2-amino acid sequence
A A S
CDR-L3-nucleotide sequence
CAC CAG GCT GAC AGT TTC CCG TAC ACT (SEQ ID NO: 200)
CDR-L3-amino acid sequence
H Q A D S F P Y T (SEQ ID NO: 201)
Heavy chain (HC)-nucleotide sequence
CTGCTGCAAGGCTCTGGCGAGGTGCAGCTGGTGGAGTCTGGGGGAGGCTTGGTACAGCCTGGGGGGTCCCTGAGACTCTC
CTGTGCAGCCTCTGGATTCACCTTTAGCAGCTATGCCATGAGCTGGGTCCGCCAGGCTCCAGGGAAGGGGCTGGAATGGG
TCTCAGCTATTAGCGGTAGTGGTGGCAGCACATACTACGCAGACTCCGTGAAGGGCCGGTTCACCATCTCCAGAGACAAT
TCCAAGAACACGCTGTATCTGCAAATGAACAGCCTGAGAGCCGAGGACACGGCCGTATATTACTGTGCGAAAGGCCTTAT
AGCACCTCGTCCGATGGGCTTTGACTACTGGGGCCAGGGAACCCTGGTCACCGTCTCCTCAGCCTCCACCAAGGGCCCAT
CGGTCTTCCCCCTGGCGCCCTGCTCCAGGAGCACCTCCGAGAGCACAGCCGCCCTGGGCTGCCTGGTCAAGGACTACTTC
CCCGAACCGGTGACGGTGTCGTGGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACGAAGACCTACACCTGCAACGTAGATC
ACAAGCCCAGCAACACCAAGGTGGACAAGAGAGTTGAGTCCAAATATGGTCCCCCATGCCCACCCTGCCCAGCACCTGAG
TTCCTGGGGGGACCATCAGTCTTCCTGTTCCCCCCAAAACCCAAGGACACTCTCATGATCTCCCGGACCCCTGAGGTCAC
GTGCGTGGTGGTGGACGTGAGCCAGGAAGACCCCGAGGTCCAGTTCAACTGGTACGTGGATGGCGTGGAGGTGCATAATG
CCAAGACAAAGCCGCGGGAGGAGCAGTTCAACAGCACGTACCGTGTGGTCAGCGTCCTCACCGTCCTGCACCAGGACTGG
CTGAACGGCAAGGAGTACAAGTGCAAGGTCTCCAACAAAGGCCTCCCGTCCTCCATCGAGAAAACCATCTCCAAAGCCAA
AGGGCAGCCCCGAGAGCCACAGGTGTACACCCTGCCCCCATCCCAGGAGGAGATGACCAAGAACCAGGTCAGCCTGACCT
GCCTGGTCAAAGGCTTCTACCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTCACCGTGGACAAGAGCAGGTGGCAGGAGGG
GAATGTCTTCTCATGCTCCGTGATGCATGAGGCTCTGCACAACCACTACACACAGAAGTCCCTCTCCCTGTCTCTGGGTA
AATGA (SEQ ID NO: 202)
Heavy chain-amino acid sequence
LLQGSGEVQLVESGGGLVQPGGSLRLSCAASGFTFSSYAMSWVRQAPGKGLEWVSAISGSGGSTYYADSVKGRFTISRDN
SKNTLYLQMNSLRAEDTAVYYCAKGLIAPRPMGFDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGK (SEQ ID NO: 203)
Light chain (LC)-nucleotide sequence
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGCAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG (SEQ ID NO: 204)
Light chain (LC)-amino acid sequence
DIQMTQSPSSVSASVGDRVTITCRASQGINSWLAWYQQKPGKAPKLLIYAASSLQSGVPSRFSGSGSGTDFTLTISSLQP
EDFATYYCHQADSFPYTFGQGTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC (SEQ ID NO: 205)
REGN8069
Heavy chain variable region (HCVR, V_{H})-nucleotide sequence
CAGGTGCAGCTGGTGGAGTCTGGGGGAGGCGTGGGCCAGCCTGGGAGGTCCCTGAGACTGTCCTGTGCAGCCTCTGGATT
CACCTTCAGCAGGAATGCCATGCACTGGGTCCGCCAGGCTCCAGGCAAGGGGCTGGAGTGGGTGGCAGTCATATCATATG
ATGGAAGTAATAAACACTATGCAGACTCCGTGAAGGGCCGATTCACCATCTCCAGAGACAATTCCAAGAACACGCTGTAT
CTGGAAATGAACAGCCTGAGAGTTGAGGACACGGCTGTGTATTATTGTGCGAAAGGGGGGATTCCTTTTGACTACTGGGG
CCAGGGAACCCTGGTCACCGTCTCCTCA (SEQ ID NO: 206)
Heavy chain variable region (HCVR, V_{H})-amino acid sequence
VQLVESGGGVGQPGRSLRLSCAASGFTFSRNAMHWVRQAPGKGLEWVAVISYDGSNKHYADSVKGRFTISRDNSKNTLY
LEMNSLRVEDTAVYYCAKGGIPFDYWGQGTLVTVSS (SEQ ID NO: 207)
CDR-H1-nucleotide sequence
GGA TTC ACC TTC AGC AGG AAT GCC (SEQ ID NO: 208)
CDR-H1-amino acid sequence
G F T F S R N A; (SEQ ID NO: 209)
CDR-H2-nucleotide sequence
ATA TCA TAT GAT GGA AGT AAT AAA (SEQ ID NO: 210)
CDR-H2-amino acid sequence
I S Y D G S N K; (SEQ ID NO: 211)
CDR-H3-nucleotide sequence
GCG AAA GGG GGG ATT CCT TTT GAC TAC (SEQ ID NO: 212)
CDR-H3-amino acid sequence
A K G G I P F D Y; (SEQ ID NO: 213)
Light chain variable region (LCVR, V_{L})-nucleotide sequence
GATATTGTGATGACCCAGTCTCCACTCTCCTCACCTGTCACCCTTGGACAGCCGGCCTCCATCTCCTGCAGGTCTAGTCA
AAGCCTCGTACACTTTGATGGAAACACCTACTTGAGTTGGCTTCACCAGAGGCCAGGCCAGCCTCCAAGACTCCTAATTT
ATAAGATTTCTAACCGCTTCTCTGGGGTCCCAGACAGATTCAGTGGCAGTGGGGCAGGGACAGATTTCACACTGAAAATC
AGCAGGGTGGAACCTGAAGATGTCGGGGTTTATTACTGCATGCATGCTACACAATTTCCGTACACTTTTGGCCAGGGGAC
CAAGCTGGAGATCAAA (SEQ ID NO: 214)
Light chain variable region (LCVR, V_{L})-amino acid sequence
DIVMTQSPLSSPVTLGQPASISCRSSQSLVHFDGNTYLSWLHQRPGQPPRLLIYKISNRFSGVPDRFSGSGAGTDFTLKI
SRVEPEDVGVYYCMHATQFPYTFGQGTKLEIK (SEQ ID NO: 215)
CDR-L1-nucleotide sequence
CAA AGC CTC GTA CAC TTT GAT GGA AAC ACC TAC (SEQ ID NO: 216)
CDR-L1-amino acid sequence
Q S L V H F D G N T Y (SEQ ID NO: 217)
CDR-L2-nucleotide sequence
AAG ATT TCT
CDR-L2-amino acid sequence
K I S
CDR-L3-nucleotide sequence
ATG CAT GCT ACA CAA TTT CCG TAC ACT (SEQ ID NO: 220)
CDR-L3-amino acid sequence
M H A T Q F P Y T (SEQ ID NO: 221)
Heavy chain (HC)-nucleotide sequence
CTGCTGCAAGGCTCTGGCCAGGTGCAGCTGGTGGAGTCTGGGGGAGGCGTGGGCCAGCCTGGGAGGTCCCTGAGACTGTC
CTGTGCAGCCTCTGGATTCACCTTCAGCAGGAATGCCATGCACTGGGTCCGCCAGGCTCCAGGCAAGGGGCTGGAGTGGG
TGGCAGTCATATCATATGATGGAAGTAATAAACACTATGCAGACTCCGTGAAGGGCCGATTCACCATCTCCAGAGACAAT
TCCAAGAACACGCTGTATCTGGAAATGAACAGCCTGAGAGTTGAGGACACGGCTGTGTATTATTGTGCGAAAGGGGGGAT
TCCTTTTGACTACTGGGGCCAGGGAACCCTGGTCACCGTCTCCTCAGCCTCCACCAAGGGCCCATCGGTCTTCCCCCTGG
CGCCCTGCTCCAGGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGCACCTGAGTTCCTGGGGGGACCA
TCAGTCTTCCTGTTCCCCCCAAAACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTCTGGGTAAATGA (SEQ ID
NO: 222)
Heavy chain-amino acid sequence
LLQGSGQVQLVESGGGVGQPGRSLRLSCAASGFTFSRNAMHWVRQAPGKGLEWVAVISYDGSNKHYADSVKGRFTISRDN
SKNTLYLEMNSLRVEDTAVYYCAKGGIPFDYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK (SEQ ID NO: 223)
Light chain (LC)-nucleotide sequence
GATATTGTGATGACCCAGTCTCCACTCTCCTCACCTGTCACCCTTGGACAGCCGGCCTCCATCTCCTGCAGGTCTAGTCA
AAGCCTCGTACACTTTGATGGAAACACCTACTTGAGTTGGCTTCACCAGAGGCCAGGCCAGCCTCCAAGACTCCTAATTT
ATAAGATTTCTAACCGCTTCTCTGGGGTCCCAGACAGATTCAGTGGCAGTGGGGCAGGGACAGATTTCACACTGAAAATC
AGCAGGGTGGAACCTGAAGATGTCGGGGTTTATTACTGCATGCATGCTACACAATTTCCGTACACTTTTGGCCAGGGGAC
CAAGCTGGAGATCAAACGAACTGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCTGAG
CAAAGCAGACTACGAGAAACACAAAGTCTACGCCTGCGAAGTCACCCATCAGGGCCTGAGCTCGCCCGTCACAAAGAGCT
TCAACAGGGGAGAGTGTTAG (SEQ ID NO: 224)
Light chain (LC)-amino acid sequence
DIVMTQSPLSSPVTLGQPASISCRSSQSLVHFDGNTYLSWLHQRPGQPPRLLIYKISNRFSGVPDRFSGSGAGTDFTLKI
SRVEPEDVGVYYCMHATQFPYTFGQGTKLEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC (SEQ ID NO: 225)
REGN8071
Heavy chain variable region (HCVR, V_{H})-nucleotide sequence
CAGGTGCAGCTGGTGGAGTCTGGGGGAGGCGTGGTCCAGCCTGCGAGGTCCCTGAGACTCTCCTGTGCAGCCTCTGGATT
CGCCTTCAGTAGGTCTGCCATGCACTGGGTCCGCCAGGCTCCAGGCAAGGGGCTGGAGTGGGTGGCAGTTATATCATATG
ATGGAAGTAATAAATACTATACAGACTCCGTGAAGGGCCGATTCACCATCTCCAGAGACAATTCCAAGAACACGCTGTAT
CTGCAAATGAACACCCTGAGAGCTGAGGACACGGCTCTTTATTACTGTGCGAAAATGTATACAACTATGGACTCTTTTGA
CTACTGGGGCCAGGGAACCCTGGTCACCGTCTCCTCA (SEQ ID NO: 226)
Heavy chain variable region (HCVR, V_{H})-amino acid sequence
QVQLVESGGGVVQPARSLRLSCAASGFAFSRSAMHWVRQAPGKGLEWVAVISYDGSNKYYTDSVKGRFTISRDNSKNTLY
LQMNTLRAEDTALYYCAKMYTTMDSFDYWGQGTLVTVSS (SEQ ID NO: 227)
CDR-H1-nucleotide sequence
GGA TTC GCC TTC AGT AGG TCT GCC (SEQ ID NO: 228)
CDR-H1-amino acid sequence
G F A F S R S A (SEQ ID NO: 229)
CDR-H2-nucleotide sequence
ATA TCA TAT GAT GGA AGT AAT AAA (SEQ ID NO: 230)
CDR-H2-amino acid sequence
I S Y D G S N K (SEQ ID NO: 231)
CDR-H3-nucleotide sequence
GCG AAA ATG TAT ACA ACT ATG GAC TCT TTT GAC TAC (SEQ ID NO: 232)
CDR-H3-amino acid sequence
A K M Y T T M D S F D Y (SEQ ID NO: 233)
Light chain variable region (LCVR, V_{L})-nucleotide sequence
GACATCCAGTTGACCCAGTCTCCATCCTTCCTGTCTGCATCTGTAGGAGACAGAGTCACCATCACTTGCTGGGCCAGTCA
GGGCATTAGCAGTTATTTAGCCTGGTATCAGCAAAAACCAGGGAAAGCCCCTAAGCTCCTGATCTATGCTGCATCCACTT
TGCAAAGTGGGGTCCCATCAAGGTTCAGCGGCAGTGGATCTGGGACAGAATTCACTCTCACAATCAGCAGCCTGCAGCCT
GAAGATTTTGCACTTTATTACTGTCAACAGCTTAATAGTTACCCTCGGACGTTCGGCCAAGGGACCAAGGTGGAAATCAA
A (SEQ ID NO: 234)
Light chain variable region (LCVR, V_{L})-amino acid sequence
DIQLTQSPSFLSASVGDRVTITCWASQGISSYLAWYQQKPGKAPKLLIYAASTLQSGVPSRFSGSGSGTEFTLTISSLQP
EDFALYYCQQLNSYPRTFGQGTKVEIK (SEQ ID NO: 235)
CDR-L1-nucleotide sequence
CAG GGC ATT AGC AGT TAT (SEQ ID NO: 236)
CDR-L1-amino acid sequence
Q G I S S Y (SEQ ID NO: 237)
CDR-L2-nucleotide sequence
GCT GCA TCC
CDR-L2-amino acid sequence
A A S
CDR-L3-nucleotide sequence
CAA CAG CTT AAT AGT TAC CCT CGG ACG (SEQ ID NO: 240)
CDR-L3-amino acid sequence
Q Q L N S Y P R T (SEQ ID NO: 241)
Heavy chain (HC)-nucleotide sequence
CTGCTGCAAGGCTCTGGCCAGGTGCAGCTGGTGGAGTCTGGGGGAGGCGTGGTCCAGCCTGCGAGGTCCCTGAGACTCTC
CTGTGCAGCCTCTGGATTCGCCTTCAGTAGGTCTGCCATGCACTGGGTCCGCCAGGCTCCAGGCAAGGGGCTGGAGTGGG
TGGCAGTTATATCATATGATGGAAGTAATAAATACTATACAGACTCCGTGAAGGGCCGATTCACCATCTCCAGAGACAAT
TCCAAGAACACGCTGTATCTGCAAATGAACACCCTGAGAGCTGAGGACACGGCTCTTTATTACTGTGCGAAAATGTATAC
AACTATGGACTCTTTTGACTACTGGGGCCAGGGAACCCTGGTCACCGTCTCCTCAGCCTCCACCAAGGGCCCATCGGTCT
TCCCCCTGGCGCCCTGCTCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCACCCTGCCCAGCACCTGAGTTCCTG
GGGGGACCATCAGTCTTCCTGTTCCCCCCAAAACCCAAGGACACTCTCATGATCTCCCGGACCCCTGAGGTCACGTGCGT
GGTGGTGGACGTGAGCCAGGAAGACCCCGAGGTCCAGTTCAACTGGTACGTGGATGGCGTGGAGGTGCATAATGCCAAGA
CAAAGCCGCGGGAGGAGCAGTTCAACAGCACGTACCGTGTGGTCAGCGTCCTCACCGTCCTGCACCAGGACTGGCTGAAC
GGCAAGGAGTACAAGTGCAAGGTCTCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGAACCAGGTCAGCCTGACCTGCCTGG
TCAAAGGCTTCTACCCCAGCGACATCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTCACCGTGGACAAGAGCAGGTGGCAGGAGGGGAATGT
CTTCTCATGCTCCGTGATGCATGAGGCTCTGCACAACCACTACACACAGAAGTCCCTCTCCCTGTCTCTGGGTAAATGA
(SEQ ID NO: 242)
Heavy chain-amino acid sequence
LLQGSGQVQLVESGGGVVQPARSLRLSCAASGFAFSRSAMHWVRQAPGKGLEWVAVISYDGSNKYYTDSVKGRFTISRDN
SKNTLYLQMNTLRAEDTALYYCAKMYTTMDSFDYWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK (SEQ ID NO: 243)
Light chain (LC)-nucleotide sequence
GACATCCAGTTGACCCAGTCTCCATCCTTCCTGTCTGCATCTGTAGGAGACAGAGTCACCATCACTTGCTGGGCCAGTCA
GGGCATTAGCAGTTATTTAGCCTGGTATCAGCAAAAACCAGGGAAAGCCCCTAAGCTCCTGATCTATGCTGCATCCACTT
TGCAAAGTGGGGTCCCATCAAGGTTCAGCGGCAGTGGATCTGGGACAGAATTCACTCTCACAATCAGCAGCCTGCAGCCT
GAAGATTTTGCACTTTATTACTGTCAACAGCTTAATAGTTACCCTCGGACGTTCGGCCAAGGGACCAAGGTGGAAATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG (SEQ ID NO: 244)
Light chain (LC)-amino acid sequence
DIQLTQSPSFLSASVGDRVTITCWASQGISSYLAWYQQKPGKAPKLLIYAASTLQSGVPSRFSGSGSGTEFTLTISSLQP
EDFALYYCQQLNSYPRTFGQGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC (SEQ ID NO: 245)
REGN9426
Heavy chain variable region (HCVR, V_{H})-nucleotide sequence
GAGGTGCAGCTGGTGGAGTCTGGGGGAGGCTTGGTCCAGCCTGGGGGGTCCCTGAAACTCTCCTGTGCAGCCTCTGGGTT
CACCTTCAGTGGCTCTGCTATGCACTGGGTCCGCCAGGCTTCCGGGAAAGGGCTGGAGTGGGTTGGCCGTATTACAAGCA
AAGCTAACAGTTACGCGACAGCATATGATGCGTCGGTGAAAGGCAGGTTCACCATCTCCAGAGATGATTCAAAGAACACG
GCGTATCTGCAAATGAACAGCCTGAAAACCGAGGACACGGCCGTGTATTACTGTACTAGGCAACGATTTTTGGAGTTTTT
ATTCCTTGACTACTGGGGCCAGGGAACCCTGGTCACCGTCTCCTCA (SEQ ID NO: 246)
Heavy chain variable region (HCVR, V_{H})-amino acid sequence
EVQLVESGGGLVQPGGSLKLSCAASGFTFSGSAMHWVRQASGKGLEWVGRITSKANSYATAYDASVKGRFTISRDDSKNT
AYLQMNSLKTEDTAVYYCTRQRFLEFLFLDYWGQGTLVTVSS (SEQ ID NO: 247)
CDR-H1-nucleotide sequence
GGG TTC ACC TTC AGT GGC TCT GCT; (SEQ ID NO: 248)
CDR-H1-amino acid sequence
G F T F S G S A (SEQ ID NO: 249)
CDR-H2-nucleotide sequence
ATT ACA AGC AAA GCT AAC AGT TAC GCG ACA (SEQ ID NO: 250)
CDR-H2-amino acid sequence
I T S K A N S Y A T (SEQ ID NO: 251)
CDR-H3-nucleotide sequence
ACT AGG CAA CGA TTT TTG GAG TTT TTA TTC CTT GAC TAC; (SEQ ID NO: 252)
CDR-H3-amino acid sequence
T R Q R F L E F L F L D Y (SEQ ID NO: 253)
Light chain variable region (LCVR, V_{L})-nucleotide sequence
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA (SEQ ID NO: 254)
Light chain variable region (LCVR, V_{L})-amino acid sequence
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK (SEQ ID NO: 255)
CDR-L1-nucleotide sequence
CAG AGC ATT AGC AGC TAT (SEQ ID NO: 256)
CDR-L1-amino acid sequence
Q S I S S Y (SEQ ID NO: 257)
CDR-L2-nucleotide sequence
GCT GCA TCC
CDR-L2-amino acid sequence
A A S
CDR-L3-nucleotide sequence
CAA CAG AGT TAC AGT ACC CCT CCG ATC ACC (SEQ ID NO: 260)
CDR-L3-amino acid sequence
Q Q S Y S T P P I T (SEQ ID NO: 261)
Heavy chain (HC)-nucleotide sequence
CTGCTGCAAGGCTCTGGCGAGGTGCAGCTGGTGGAGTCTGGGGGAGGCTTGGTCCAGCCTGGGGGGTCCCTGAAACTCTC
CTGTGCAGCCTCTGGGTTCACCTTCAGTGGCTCTGCTATGCACTGGGTCCGCCAGGCTTCCGGGAAAGGGCTGGAGTGGG
TTGGCCGTATTACAAGCAAAGCTAACAGTTACGCGACAGCATATGATGCGTCGGTGAAAGGCAGGTTCACCATCTCCAGA
GATGATTCAAAGAACACGGCGTATCTGCAAATGAACAGCCTGAAAACCGAGGACACGGCCGTGTATTACTGTACTAGGCA
ACGATTTTTGGAGTTTTTATTCCTTGACTACTGGGGCCAGGGAACCCTGGTCACCGTCTCCTCAGCCTCCACCAAGGGCC
CATCGGTCTTCCCCCTGGCGCCCTGCTCCAGGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACGAAGACCTACACCTGCAACGTAG
ATCACAAGCCCAGCAACACCAAGGTGGACAAGAGAGTTGAGTCCAAATATGGTCCCCCATGCCCACCCTGCCCAGCACCT
GAGTTCCTGGGGGGACCATCAGTCTTCCTGTTCCCCCCAAAACCCAAGGACACTCTCATGATCTCCCGGACCCCTGAGGT
CACGTGCGTGGTGGTGGACGTGAGCCAGGAAGACCCCGAGGTCCAGTTCAACTGGTACGTGGATGGCGTGGAGGTGCATA
ATGCCAAGACAAAGCCGCGGGAGGAGCAGTTCAACAGCACGTACCGTGTGGTCAGCGTCCTCACCGTCCTGCACCAGGAC
TGGCTGAACGGCAAGGAGTACAAGTGCAAGGTCTCCAACAAAGGCCTCCCGTCCTCCATCGAGAAAACCATCTCCAAAGC
CAAAGGGCAGCCCCGAGAGCCACAGGTGTACACCCTGCCCCCATCCCAGGAGGAGATGACCAAGAACCAGGTCAGCCTGA
CCTGCCTGGTCAAAGGCTTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTCACCGTGGACAAGAGCAGGTGGCAGGA
GGGGAATGTCTTCTCATGCTCCGTGATGCATGAGGCTCTGCACAACCACTACACACAGAAGTCCCTCTCCCTGTCTCTGG
GTAAATGA (SEQ ID NO: 262)
Heavy chain-amino acid sequence
LLQGSGEVQLVESGGGLVQPGGSLKLSCAASGFTFSGSAMHWVRQASGKGLEWVGRITSKANSYATAYDASVKGRFTISR
DDSKNTAYLQMNSLKTEDTAVYYCTRQRFLEFLFLDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYTCNVDHKPSNTKVDKRVESKYGPPCPPCPAP
EFLGGPSVFLFPPKPKDTLMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK (SEQ ID NO: 263)
Light chain (LC)-nucleotide sequence
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG (SEQ ID NO: 264)
Light chain (LC)-amino acid sequence
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC (SEQ ID NO: 265)
REGN5203
Heavy chain (HC)-nucleotide sequence
TTACTTCAGGGATCTGGTCAAGTTACTCTTAAAGAATCTGGTCCTGGAATTCTTCAACCTTCTCAAACTCTTTCTCTTAC
TTGTTCTTTTTCTGGTTTTTCTCTTTCTACTTCTGGTACTGGTGTTGGTTGGATTCGTCAACCTTCTGGTAAAGGTCTTG
AATGGCTTTCTCATATTTGGTGGGATGATGTTAAACGTTATAATCCTGCTCTTAAATCTCGTCTTACTATTTCTCGTGAT
ACTTCTTATTCTCAAGTTTTTCTTCGTATTGCTTCTGTTGATACTGCTGATACTGCTACTTATTATTGTGCTCGTATTCT
TGATGGTACTGGTCCTATGGATTATTGGGGTCAAGGTACTTCTGTTACTGTTTCTTCTGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA (SEQ ID NO: 266)
Heavy chain-amino acid sequence
LLQGSGQVTLKESGPGILQPSQTLSLTCSFSGFSLSTSGTGVGWIRQPSGKGLEWLSHIWWDDVKRYNPALKSRLTISRD
TSYSQVFLRIASVDTADTATYYCARILDGTGPMDYWGQGTSVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK (SEQ ID NO: 267)
Light chain (LC)-nucleotide sequence
CAAATTGTTCTTACTCAATCTCCTGCTATTATGTCTGCTAGCCCTGGTGAAAAAGTTACTATGACTTGTTCTGCTTCTTC
TCGTGTTACTTATATGCATTGGTATCAACAACGTTCTGGTACTTCTCCTAAACGTTGGATTTATGATACTTCTAAACTTG
CTTCTGGTGTTCCTGCTCGTTTTTCTGGTTCTGGTTCTGGTACTTCTTATTCTCTTACTATTTCTTCTATGGAAGCTGAA
GATGCTGCTACTTATTATTGTCAACAATGGGGTAATAATCCTCAATATACTTTTGGTGGTGGTACTCGTCTTGAAATTAA
ACGTCGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG (SEQ ID NO: 268)
Light chain (LC)-amino acid sequence
QIVLTQSPAIMSASPGEKVTMTCSASSRVTYMHWYQQRSGTSPKRWIYDTSKLASGVPARFSGSGSGTSYSLTISSMEAE
DAATYYCQQWGNNPQYTFGGGTRLEIKRRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC (SEQ ID NO: 269)
REGN5204
Heavy chain (HC)-nucleotide sequence
CAAGTTACTCTTAAAGAATCTGGTCCTGGAATTCTTCAACCTTCTCAAACTCTTTCTCTTACTTGTTCTTTTTCTGGTTT
TTCTCTTTCTACTTCTGGTACTGGTGTTGGTTGGATTCGTCAACCTTCTGGTAAAGGTCTTGAATGGCTTTCTCATATTT
GGTGGGATGATGTTAAACGTTATAATCCTGCTCTTAAATCTCGTCTTACTATTTCTCGTGATACTTCTTATTCTCAAGTT
TTTCTTCGTATTGCTTCTGTTGATACTGCTGATACTGCTACTTATTATTGTGCTCGTATTCTTGATGGTACTGGTCCTAT
GGATTATTGGGGTCAAGGTACTTCTGTTACTGTTTCTTCTGCCTCCACCAAGGGCCCATCGGTCTTCCCCCTGGCGCCCT
GCTCCAGGAGCACCTCCGAGAGCACAGCCGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGTAGATCACAAGCCCAGCAACACCAAGG
TGGACAAGAGAGTTGAGTCCAAATATGGTCCCCCATGCCCACCCTGCCCAGCACCTGAGTTCCTGGGGGGACCATCAGTC
TTCCTGTTCCCCCCAAAACCCAAGGACACTCTCATGATCTCCCGGACCCCTGAGGTCACGTGCGTGGTGGTGGACGTGAG
CCAGGAAGACCCCGAGGTCCAGTTCAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGACCTGCCTGGTCAAAGGCTTCTACC
CCAGCGACATCGCCGTGGAGTGGGAGAGCAATGGGCAGCCGGAGAACAACTACAAGACCACGCCTCCCGTGCTGGACTCC
GACGGCTCCTTCTTCCTCTACAGCAGGCTCACCGTGGACAAGAGCAGGTGGCAGGAGGGGAATGTCTTCTCATGCTCCGT
GATGCATGAGGCTCTGCACAACCACTACACACAGAAGTCCCTCTCCCTGTCTCTGGGTAAATGA (SEQ ID NO: 270)
Heavy chain-amino acid sequence
QVTLKESGPGILQPSQTLSLTCSFSGFSLSTSGTGVGWIRQPSGKGLEWLSHIWWDDVKRYNPALKSRLTISRDTSYSQV
FLRIASVDTADTATYYCARILDGTGPMDYWGQGTSVTVSSASTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK (SEQ ID NO: 271)
Light chain (LC)-nucleotide sequence
TTACTTCAGGGATCTGGTCAAATTGTTCTTACTCAATCTCCTGCTATTATGTCTGCTAGCCCTGGTGAAAAAGTTACTAT
GACTTGTTCTGCTTCTTCTCGTGTTACTTATATGCATTGGTATCAACAACGTTCTGGTACTTCTCCTAAACGTTGGATTT
ATGATACTTCTAAACTTGCTTCTGGTGTTCCTGCTCGTTTTTCTGGTTCTGGTTCTGGTACTTCTTATTCTCTTACTATT
TCTTCTATGGAAGCTGAAGATGCTGCTACTTATTATTGTCAACAATGGGGTAATAATCCTCAATATACTTTTGGTGGTGG
TACTCGTCTTGAAATTAAACGTCGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG (SEQ ID NO: 272)
Light chain (LC)-amino acid sequence
LLQGSGQIVLTQSPAIMSASPGEKVTMTCSASSRVTYMHWYQQRSGTSPKRWIYDTSKLASGVPARFSGSGSGTSYSLTI
SSMEAEDAATYYCQQWGNNPQYTFGGGTRLEIKRRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC (SEQ ID NO: 273)
REGN5617
Heavy chain variable region (HCVR, V_{H})-nucleotide sequence
GAGGTGCAGCTGGTGGAGTCTGGGGGAGGCTTGGTCCAGCCTGGGGGGTCCCTGAGACTCTCCTGTGCAGCCTCTGGATT
CACCTTCTCTAGGTACTGGATGACCTGGGTCCGCCAGGCTCCAGGGAAGGGGCTGGAGTGGGTGGCCAACATAAAGCAAG
ATGGCAGTGGGAAAAACTATGTGGACTCTGTGATGGGCCGATACACCATCTCCAGAGACAACGCCAAGAACTCACTGTAT
CTGCAAATGAACAGCCTGAGAGCCGAGGACACGGCTGTGTATTACTGTGCGAGATGGATAGCACCAGATTTCCCCGGTAT
GGACGTCTGGGGCCAAGGGACCACGGTCACCGTCTCCTCA (SEQ ID NO: 274)
Heavy chain variable region (HCVR, V_{H})-amino acid sequence
EVQLVESGGGLVQPGGSLRLSCAASGFTFSRYWMTWVRQAPGKGLEWVANIKQDGSGKNYVDSVMGRYTISRDNAKNSLY
LQMNSLRAEDTAVYYCARWIAPDFPGMDVWGQGTTVTVSS (SEQ ID NO: 275)
CDR-H1-nucleotide sequence
GGA TTC ACC TTC TCT AGG TAC TGG (SEQ ID NO: 276)
CDR-H1-amino acid sequence
G F T F S R Y W (SEQ ID NO: 277)
CDR-H2-nucleotide sequence
ATA AAG CAA GAT GGC AGT GGG AAA (SEQ ID NO: 278)
CDR-H2-amino acid sequence
I K Q D G S G K (SEQ ID NO: 279)
CDR-H3-nucleotide sequence
GCG AGA TGG ATA GCA CCA GAT TTC CCC GGT ATG GAC GTC (SEQ ID NO: 280)
CDR-H3-amino acid sequence
A R W I A P D F P G M D V (SEQ ID NO: 281)
Light chain variable region (LCVR, V_{L})-nucleotide sequence
GACATCCAGTTGACCCAGTCTCCATCCTTCCTGTCTGCATCTGTAGGAGACAGAGTCACCATCACTTGCTGGGCCAGTCA
GGGCATTAGCAGTTATTTAGCCTGGTATCAGCAAAAACCAGGAAAAGCCCCTAAGCTCCTGATCTATGCTGCATCCACTT
TGCAAAGTGGGGTCCCATCAAGGTTCAGCGGCAGTGGATCTGGGACAGAATTCACTCTCACAATCAGCAGCCTGCAGCCT
GCAGATTTTGCAACTTATTACTGTCAACAGCTTAATAGTTACCCGCTCACTTTCGGCGGAGGGACCAAGGTGGAGATCAA
A (SEQ ID NO: 282)
Light chain variable region (LCVR, V_{L})-amino acid sequence
DIQLTQSPSFLSASVGDRVTITCWASQGISSYLAWYQQKPGKAPKLLIYAASTLQSGVPSRFSGSGSGTEFTLTISSLQP
ADFATYYCQQLNSYPLTFGGGTKVEIK (SEQ ID NO: 283)
CDR-L1-nucleotide sequence
CAG GGC ATT AGC AGT TAT (SEQ ID NO: 284)
CDR-L1-amino acid sequence
Q G I S S Y (SEQ ID NO: 285)
CDR-L2-nucleotide sequence
GCT GCA TCC
CDR-L2-amino acid sequence
A A S
CDR-L3-nucleotide sequence
CAA CAG CTT AAT AGT TAC CCG CTC ACT (SEQ ID NO: 288)
CDR-L3-amino acid sequence
Q Q L N S Y P L T (SEQ ID NO: 289)
Heavy chain (HC)-nucleotide sequence
GAGGTGCAGCTGGTGGAGTCTGGGGGAGGCTTGGTCCAGCCTGGGGGGTCCCTGAGACTCTCCTGTGCAGCCTCTGGATT
CACCTTCTCTAGGTACTGGATGACCTGGGTCCGCCAGGCTCCAGGGAAGGGGCTGGAGTGGGTGGCCAACATAAAGCAAG
ATGGCAGTGGGAAAAACTATGTGGACTCTGTGATGGGCCGATACACCATCTCCAGAGACAACGCCAAGAACTCACTGTAT
CTGCAAATGAACAGCCTGAGAGCCGAGGACACGGCTGTGTATTACTGTGCGAGATGGATAGCACCAGATTTCCCCGGTAT
GGACGTCTGGGGCCAAGGGACCACGGTCACCGTCTCCTCAGCCTCCACCAAGGGCCCATCGGTCTTCCCCCTGGCGCCCT
GCTCCAGGAGCACCTCCGAGAGCACAGCCGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGTAGATCACAAGCCCAGCAACACCAAGG
TGGACAAGAGAGTTGAGTCCAAATATGGTCCCCCATGCCCACCCTGCCCAGCACCTGAGTTCCTGGGGGGACCATCAGTC
TTCCTGTTCCCCCCAAAACCCAAGGACACTCTCATGATCTCCCGGACCCCTGAGGTCACGTGCGTGGTGGTGGACGTGAG
CCAGGAAGACCCCGAGGTCCAGTTCAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGACCTGCCTGGTCAAAGGCTTCTACC
CCAGCGACATCGCCGTGGAGTGGGAGAGCAATGGGCAGCCGGAGAACAACTACAAGACCACGCCTCCCGTGCTGGACTCC
GACGGCTCCTTCTTCCTCTACAGCAGGCTCACCGTGGACAAGAGCAGGTGGCAGGAGGGGAATGTCTTCTCATGCTCCGT
GATGCATGAGGCTCTGCACAACCACTACACACAGAAGTCCCTCTCCCTGTCTCTGGGTAAATGA (SEQ ID NO: 290)
Heavy chain-amino acid sequence
EVQLVESGGGLVQPGGSLRLSCAASGFTFSRYWMTWVRQAPGKGLEWVANIKQDGSGKNYVDSVMGRYTISRDNAKNSLY
LQMNSLRAEDTAVYYCARWIAPDFPGMDVWGQGTTVTVSSASTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK (SEQ ID NO: 291)
Light chain (LC)-nucleotide sequence
CTGCTGCAAGGCTCTGGCGACATCCAGTTGACCCAGTCTCCATCCTTCCTGTCTGCATCTGTAGGAGACAGAGTCACCAT
CACTTGCTGGGCCAGTCAGGGCATTAGCAGTTATTTAGCCTGGTATCAGCAAAAACCAGGAAAAGCCCCTAAGCTCCTGA
TCTATGCTGCATCCACTTTGCAAAGTGGGGTCCCATCAAGGTTCAGCGGCAGTGGATCTGGGACAGAATTCACTCTCACA
ATCAGCAGCCTGCAGCCTGCAGATTTTGCAACTTATTACTGTCAACAGCTTAATAGTTACCCGCTCACTTTCGGCGGAGG
GACCAAGGTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG (SEQ ID NO: 292)
Light chain (LC)-amino acid sequence
LLQGSGDIQLTQSPSFLSASVGDRVTITCWASQGISSYLAWYQQKPGKAPKLLIYAASTLQSGVPSRFSGSGSGTEFTLT
ISSLQPADFATYYCQQLNSYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC (SEQ ID NO: 293)
REGN5619
Heavy chain variable region (HCVR, V_{H})-nucleotide sequence
GAGGTGCAGCTGGTGGAGTCTGGGGGAGGCTTGGTCCAGCCTGGGGGGTCCCTGAAACTCTCCTGTGCAGCCTCTGGGTT
CACCTTCAGTGGCTCTGCTATGCACTGGGTCCGCCAGGCTTCCGGGAAAGGGCTGGAGTGGGTTGGCCGTATTACAAGCA
AAGCTAACAGTTACGCGACAGCATATGATGCGTCGGTGAAAGGCAGGTTCACCATCTCCAGAGATGATTCAAAGAACACG
GCGTATCTGCAAATGAACAGCCTGAAAACCGAGGACACGGCCGTGTATTACTGTACTAGGCAACGATTTTTGGAGTTTTT
ATTCCTTGACTACTGGGGCCAGGGAACCCTGGTCACCGTCTCCTCA (SEQ ID NO: 294)
Heavy chain variable region (HCVR, V_{H})-amino acid sequence
EVQLVESGGGLVQPGGSLKLSCAASGFTFSGSAMHWVRQASGKGLEWVGRITSKANSYATAYDASVKGRFTISRDDSKNT
AYLQMNSLKTEDTAVYYCTRQRFLEFLFLDYWGQGTLVTVSS (SEQ ID NO: 295)
CDR-H1-nucleotide sequence
GGG TTC ACC TTC AGT GGC TCT GCT (SEQ ID NO: 296)
CDR-H1-amino acid sequence
G F T F S G S A (SEQ ID NO: 297)
CDR-H2-nucleotide sequence
ATT ACA AGC AAA GCT AAC AGT TAC GCG ACA (SEQ ID NO: 298)
CDR-H2-amino acid sequence
I T S K A N S Y A T (SEQ ID NO: 299)
CDR-H3-nucleotide sequence
ACT AGG CAA CGA TTT TTG GAG TTT TTA TTC CTT GAC TAC (SEQ ID NO: 300)
CDR-H3-amino acid sequence
T R Q R F L E F L F L D Y (SEQ ID NO: 301)
Light chain variable region (LCVR, V_{L})-nucleotide sequence
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA (SEQ ID NO: 302)
Light chain variable region (LCVR, V_{L})-amino acid sequence
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK (SEQ ID NO: 303)
CDR-L1-nucleotide sequence
CAG AGC ATT AGC AGC TAT (SEQ ID NO: 304)
CDR-L1-amino acid sequence
Q S I S S Y (SEQ ID NO: 305)
CDR-L2-nucleotide sequence
GCT GCA TCC
CDR-L2-amino acid sequence
A A S
CDR-L3-nucleotide sequence
CAA CAG AGT TAC AGT ACC CCT CCG ATC ACC; (SEQ ID NO: 308)
CDR-L3-amino acid sequence
Q Q S Y S T P P I T (SEQ ID NO: 309)
Heavy chain (HC)-nucleotide sequence
GAGGTGCAGCTGGTGGAGTCTGGGGGAGGCTTGGTCCAGCCTGGGGGGTCCCTGAAACTCTCCTGTGCAGCCTCTGGGTT
CACCTTCAGTGGCTCTGCTATGCACTGGGTCCGCCAGGCTTCCGGGAAAGGGCTGGAGTGGGTTGGCCGTATTACAAGCA
AAGCTAACAGTTACGCGACAGCATATGATGCGTCGGTGAAAGGCAGGTTCACCATCTCCAGAGATGATTCAAAGAACACG
GCGTATCTGCAAATGAACAGCCTGAAAACCGAGGACACGGCCGTGTATTACTGTACTAGGCAACGATTTTTGGAGTTTTT
ATTCCTTGACTACTGGGGCCAGGGAACCCTGGTCACCGTCTCCTCAGCCTCCACCAAGGGCCCATCGGTCTTCCCCCTGG
CGCCCTGCTCCAGGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGCACCTGAGTTCCTGGGGGGACCA
TCAGTCTTCCTGTTCCCCCCAAAACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTCTGGGTAAATGA (SEQ ID
NO: 310)
Heavy chain-amino acid sequence
EVQLVESGGGLVQPGGSLKLSCAASGFTFSGSAMHWVRQASGKGLEWVGRITSKANSYATAYDASVKGRFTISRDDSKNT
AYLQMNSLKTEDTAVYYCTRQRFLEFLFLDYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK (SEQ ID NO: 311)
Light chain (LC)-nucleotide sequence
CTGCTGCAAGGCTCTGGCGACATCCAGATGACCCAGTCTCCATCCTCCCTGTCTGCATCTGTAGGAGACAGAGTCACCAT
CACTTGCCGGGCAAGTCAGAGCATTAGCAGCTATTTAAATTGGTATCAGCAGAAACCAGGGAAAGCCCCTAAGCTCCTGA
TCTATGCTGCATCCAGTTTGCAAAGTGGGGTCCCGTCAAGGTTCAGTGGCAGTGGATCTGGGACAGATTTCACTCTCACC
ATCAGCAGTCTGCAACCTGAAGATTTTGCAACTTACTACTGTCAACAGAGTTACAGTACCCCTCCGATCACCTTCGGCCA
AGGGACACGACTGGAGATTAAACGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG (SEQ ID NO: 312)
Light chain (LC)-amino acid sequence
LLQGSGDIQMTQSPSSLSASVGDRVTITCRASQSISSYLNWYQQKPGKAPKLLIYAASSLQSGVPSRFSGSGSGTDFTLT
ISSLQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC (SEQ ID NO: 313)
REGN7987
Heavy chain variable region (HCVR, V_{H})-nucleotide sequence
CAGGTGCAGCTGGTGGAGTCTGGGGGAGGCGTGGTCCAGCCTGGGAGGTCCCTGAGACTCTCCTGTGCAGCGTCTGGATT
CACCTTCAGTGGCTATGGCATACACTGGGTCCGCCAGGCTCCAGGCAAGGGACTGGTGTGGGTGGCAGTTATATGGTATG
ATGGAAGTTTTAAATACTATGCAGACTCCGTGAAGGGCCGATTCACCATCTCCAGAGACAATTCCAAGAACACGCTGTAT
CTGCAGATGAACAGCCTGAGAGCCGAGGACACGGCTGTGTATTACTGTGCGAGAGGTGATAGCAGCTCGTCCGGACGGTA
CTACTACTACGGTATGGACGTCTGGGGCCAAGGGACCACGGTCACCGTCTCCTCA (SEQ ID NO: 314)
Heavy chain variable region (HCVR, V_{H})-amino acid sequence
QVQLVESGGGVVQPGRSLRLSCAASGFTFSGYGIHWVRQAPGKGLVWVAVIWYDGSFKYYADSVKGRFTISRDNSKNTLY
LQMNSLRAEDTAVYYCARGDSSSSGRYYYYGMDVWGQGTTVTVSS; (SEQ ID NO: 315)
CDR-H1-nucleotide sequence
GA TTC ACC TTC AGT GGC TAT GGC (SEQ ID NO: 316)
CDR-H1-amino acid sequence
G F T F S G Y G (SEQ ID NO: 317)
CDR-H2-nucleotide sequence
ATA TGG TAT GAT GGA AGT TTT AAA (SEQ ID NO: 318)
CDR-H2-amino acid sequence
I W Y D G S F K (SEQ ID NO: 319)
CDR-H3-nucleotide sequence
GCG AGA GGT GAT AGC AGC TCG TCC GGA CGG TAC TAC TAC TAC GGT ATG GAC GTC (SEQ ID
NO: 320)
CDR-H3-amino acid sequence
A R G D S S S S G R Y Y Y Y G M D V (SEQ ID NO:
321)
Light chain variable region (LCVR, V_{L})-nucleotide sequence
GACATCCAGATGACCCAGTCTCCATCCTCCCTGTCTGCATCTGTAGGAGACAGAGTCACCATCACTTGCCGGGCAAGTCA
GGGCATTAGAAATGATTTAGGCTGGTATCAGCAGAAACCAGGGACAGCCCCTAAGCGCCTGATCTTTGCTGCATCCAGTT
TGCAAAGTGGGGTCCCATCAAGGTTCAGCGGCAGTGGATCTGGGACAGAATTCACTCTCACAATCAGCAGCCTGCAGCCT
GAAGATTTTGCGACTTATTACTGTCTACAGCATAATAATTACCCTCCCACTTTCGGCGGAGGGACCAAGGTGGAGATCAA
A (SEQ ID NO: 322)
Light chain variable region (LCVR, V_{L})-amino acid sequence
DIQMTQSPSSLSASVGDRVTITCRASQGIRNDLGWYQQKPGTAPKRLIFAASSLQSGVPSRFSGSGSGTEFTLTISSLQP
EDFATYYCLQHNNYPPTFGGGTKVEIK (SEQ ID NO: 323)
CDR-L1-nucleotide sequence
CAG GGC ATT AGA AAT GAT (SEQ ID NO: 324)
CDR-L1-amino acid sequence
Q G I R N D (SEQ ID NO: 325)
CDR-L2-nucleotide sequence
GCT GCA TCC
CDR-L2-amino acid sequence
A A S
CDR-L3-nucleotide sequence
CTA CAG CAT AAT AAT TAC CCT CCC ACT (SEQ ID NO: 328)
CDR-L3-amino acid sequence
L Q H N Y P P T (SEQ ID NO: 329)
Heavy chain (HC)-nucleotide sequence
CTGCTGCAAGGCTCTGGCCAGGTGCAGCTGGTGGAGTCTGGGGGAGGCGTGGTCCAGCCTGGGAGGTCCCTGAGACTCTC
CTGTGCAGCGTCTGGATTCACCTTCAGTGGCTATGGCATACACTGGGTCCGCCAGGCTCCAGGCAAGGGACTGGTGTGGG
TGGCAGTTATATGGTATGATGGAAGTTTTAAATACTATGCAGACTCCGTGAAGGGCCGATTCACCATCTCCAGAGACAAT
TCCAAGAACACGCTGTATCTGCAGATGAACAGCCTGAGAGCCGAGGACACGGCTGTGTATTACTGTGCGAGAGGTGATAG
CAGCTCGTCCGGACGGTACTACTACTACGGTATGGACGTCTGGGGCCAAGGGACCACGGTCACCGTCTCCTCAGCCTCCA
CCAAGGGCCCATCGGTCTTCCCCCTGGCGCCCTGCTCCAGGAGCACCTCCGAGAGCACAGCCGCCCTGGGCTGCCTGGTC
AAGGACTACTTCCCCGAACCGGTGACGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGCAGCTTGGGCACGAAGACCTACACCT
GCAACGTAGATCACAAGCCCAGCAACACCAAGGTGGACAAGAGAGTTGAGTCCAAATATGGTCCCCCATGCCCACCCTGC
CCAGCACCTGAGTTCCTGGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTGGTACGTGGATGGCGTGG
AGGTGCATAATGCCAAGACAAAGCCGCGGGAGGAGCAGTTCAACAGCACGTACCGTGTGGTCAGCGTCCTCACCGTCCTG
CACCAGGACTGGCTGAACGGCAAGGAGTACAAGTGCAAGGTCTCCAACAAAGGCCTCCCGTCCTCCATCGAGAAAACCAT
CTCCAAAGCCAAAGGGCAGCCCCGAGAGCCACAGGTGTACACCCTGCCCCCATCCCAGGAGGAGATGACCAAGAACCAGG
TCAGCCTGACCTGCCTGGTCAAAGGCTTCTACCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTACAGCAGGCTCACCGTGGACAAGAGCAG
GTGGCAGGAGGGGAATGTCTTCTCATGCTCCGTGATGCATGAGGCTCTGCACAACCACTACACACAGAAGTCCCTCTCCC
TGTCTCTGGGTAAATGA (SEQ ID NO: 330)
Heavy chain-amino acid sequence
LLQGSGQVQLVESGGGVVQPGRSLRLSCAASGFTFSGYGIHWVRQAPGKGLVWVAVIWYDGSFKYYADSVKGRFTISRDN
SKNTLYLQMNSLRAEDTAVYYCARGDSSSSGRYYYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTKTYTCNVDHKPSNTKVDKRVESKYGPPCPPC
PAPEFLGGPSVFLFPPKPKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK (SEQ ID NO: 331)
Light chain (LC)-nucleotide sequence
GACATCCAGATGACCCAGTCTCCATCCTCCCTGTCTGCATCTGTAGGAGACAGAGTCACCATCACTTGCCGGGCAAGTCA
GGGCATTAGAAATGATTTAGGCTGGTATCAGCAGAAACCAGGGACAGCCCCTAAGCGCCTGATCTTTGCTGCATCCAGTT
TGCAAAGTGGGGTCCCATCAAGGTTCAGCGGCAGTGGATCTGGGACAGAATTCACTCTCACAATCAGCAGCCTGCAGCCT
GAAGATTTTGCGACTTATTACTGTCTACAGCATAATAATTACCCTCCCACTTTCGGCGGAGGGACCAAGGTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG; (SEQ ID NO: 332)
Light chain (LC)-amino acid sequence
DIQMTQSPSSLSASVGDRVTITCRASQGIRNDLGWYQQKPGTAPKRLIFAASSLQSGVPSRFSGSGSGTEFTLTISSLQP
EDFATYYCLQHNNYPPTFGGGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC (SEQ ID NO: 333)
REGN9270
Heavy chain variable region (HCVR, V_{H})-nucleotide sequence
CAGGTGCAGCTACAGCAGTGGGGCGCAGGACTGTTGAAGCCTTCGGAGACCCTGTCCCTCACCTGCGCTGTCTATGGTGG
GTCCTTCAGTGGTTACTACTGGAGCTGGATCCGCCAGCCCCCAGGAAAGGGGCTGGAGTGGATTGGGGAAATCAATCATG
CTGGAAGCACCAACTACAACCCGTCCCTCAAGAGTCGAATCACCATATCAGTGGACACGTCCAAGAACCAGTTCTCCCTG
AAGCTGAGTTCTGTGACCGCCGCGGACACGGCTGTGTATTACTGTGCGAGAGGATGGTACTATGGTTCGGGGAGTTATCA
CCGAAACTGGTTCGACCCCTGGGGCCAGGGAACCCTGGTCACCGTCTCCTCA (SEQ ID NO: 334)
Heavy chain variable region (HCVR, V_{H})-amino acid sequence
QVQLQQWGAGLLKPSETLSLTCAVYGGSFSGYYWSWIRQPPGKGLEWIGEINHAGSTNYNPSLKSRITISVDTSKNQFSL
KLSSVTAADTAVYYCARGWYYGSGSYHRNWFDPWGQGTLVTVSS (SEQ ID NO: 335)
CDR-H1-nucleotide sequence
GGT GGG TCC TTC AGT GGT TAC TAC (SEQ ID NO: 336)
CDR-H1-amino acid sequence
G G S F S G Y Y (SEQ ID NO: 337)
CDR-H2-nucleotide sequence
ATC AAT CAT GCT GGA AGC ACC (SEQ ID NO: 338)
CDR-H2-amino acid sequence
I N H A G S T (SEQ ID NO: 339)
CDR-H3-nucleotide sequence
GCG AGA GGA TGG TAC TAT GGT TCG GGG AGT TAT CAC CGA AAC TGG TTC GAC CCC (SEQ ID
NO: 340)
CDR-H3-amino acid sequence
A R G W Y Y G S G S Y H R N W F D P (SEQ ID NO:
341)
Light chain variable region (LCVR, V_{L})-nucleotide sequence
GAAATTGTATTGACGCAGTCTCCAGGCACCCTGTCTTTGTCTCCAGGGGAAAGAGCCACCCTCTCCTGCAGGGCCAGTCA
GAGTGTTTATTACAGCTACTTAGCCTGGTATCAGCAGAAACCTGGCCAGGCTCCCAGGCTCCTCATCTATGGTGCATCCA
ACAGGGCCACTGGCATCCCAGACAGGTTCAGTGGCAGTGGGTCTGGGACAGACTTCACTCTCACCATCAGCAGACTGGAG
CCTGAAGATTTTGCAGTGTATTACTGTCAGCAGTATGGTAACTCACCTTGGACGTTCGGCCAAGGGACCAAGGTGGAAAT
CAAA (SEQ ID NO: 342)
Light chain variable region (LCVR, V_{L})-amino acid sequence
EIVLTQSPGTLSLSPGERATLSCRASQSVYYSYLAWYQQKPGQAPRLLIYGASNRATGIPDRFSGSGSGTDFTLTISRLE
PEDFAVYYCQQYGNSPWTFGQGTKVEIK (SEQ ID NO: 343)
CDR-L1-nucleotide sequence
CAG AGT GTT TAT TAC AGC TAC (SEQ ID NO: 344)
CDR-L1-amino acid sequence
Q S V Y Y S Y (SEQ ID NO: 345)
CDR-L2-nucleotide sequence
GGT GCA TCC
CDR-L2-amino acid sequence
G A S
CDR-L3-nucleotide sequence
CAG CAG TAT GGT AAC TCA CCT TGG ACG (SEQ ID NO: 348)
CDR-L3-amino acid sequence
Q Q Y G N S P W T (SEQ ID NO: 349)
Heavy chain (HC)-nucleotide sequence
TTACTTCAGGGATCTGGTCAGGTGCAGCTACAGCAGTGGGGCGCAGGACTGTTGAAGCCTTCGGAGACCCTGTCCCTCAC
CTGCGCTGTCTATGGTGGGTCCTTCAGTGGTTACTACTGGAGCTGGATCCGCCAGCCCCCAGGAAAGGGGCTGGAGTGGA
TTGGGGAAATCAATCATGCTGGAAGCACCAACTACAACCCGTCCCTCAAGAGTCGAATCACCATATCAGTGGACACGTCC
AAGAACCAGTTCTCCCTGAAGCTGAGTTCTGTGACCGCCGCGGACACGGCTGTGTATTACTGTGCGAGAGGATGGTACTA
TGGTTCGGGGAGTTATCACCGAAACTGGTTCGACCCCTGGGGCCAGGGAACCCTGGTCACCGTCTCCTCAGCCTCCACCA
AGGGCCCATCGGTCTTCCCCCTGGCGCCCTGCTCCAGGAGCACCTCCGAGAGCACAGCCGCCCTGGGCTGCCTGGTCAAG
GACTACTTCCCCGAACCGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACGAAGACCTACACCTGCA
ACGTAGATCACAAGCCCAGCAACACCAAGGTGGACAAGAGAGTTGAGTCCAAATATGGTCCCCCATGCCCACCCTGCCCA
GCACCTGAGTTCCTGGGGGGACCATCAGTCTTCCTGTTCCCCCCAAAACCCAAGGACACTCTCATGATCTCCCGGACCCC
TGAGGTCACGTGCGTGGTGGTGGACGTGAGCCAGGAAGACCCCGAGGTCCAGTTCAACTGGTACGTGGATGGCGTGGAGG
TGCATAATGCCAAGACAAAGCCGCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCCCAGGAGGAGATGACCAAGAACCAGGTCA
GCCTGACCTGCCTGGTCAAAGGCTTCTACCCCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTACAGCAGGCTCACCGTGGACAAGAGCAGGTG
GCAGGAGGGGAATGTCTTCTCATGCTCCGTGATGCATGAGGCTCTGCACAACCACTACACACAGAAGTCCCTCTCCCTGT
CTCTGGGTAAATGA (SEQ ID NO: 350)
Heavy chain-amino acid sequence
LLQGSGQVQLQQWGAGLLKPSETLSLTCAVYGGSFSGYYWSWIRQPPGKGLEWIGEINHAGSTNYNPSLKSRITISVDTS
KNQFSLKLSSVTAADTAVYYCARGWYYGSGSYHRNWFDPWGQGTLVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK (SEQ ID NO: 351)
Light chain (LC)-nucleotide sequence
GAAATTGTATTGACGCAGTCTCCAGGCACCCTGTCTTTGTCTCCAGGGGAAAGAGCCACCCTCTCCTGCAGGGCCAGTCA
GAGTGTTTATTACAGCTACTTAGCCTGGTATCAGCAGAAACCTGGCCAGGCTCCCAGGCTCCTCATCTATGGTGCATCCA
ACAGGGCCACTGGCATCCCAGACAGGTTCAGTGGCAGTGGGTCTGGGACAGACTTCACTCTCACCATCAGCAGACTGGAG
CCTGAAGATTTTGCAGTGTATTACTGTCAGCAGTATGGTAACTCACCTTGGACGTTCGGCCAAGGGACCAAGGTGGAAAT
CAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG (SEQ ID NO: 352)
Light chain (LC)-amino acid sequence
EIVLTQSPGTLSLSPGERATLSCRASQSVYYSYLAWYQQKPGQAPRLLIYGASNRATGIPDRFSGSGSGTDFTLTISRLE
PEDFAVYYCQQYGNSPWTFGQGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC (SEQ ID NO: 353)
REGN9278
Heavy chain variable region (HCVR, V_{H})-nucleotide sequence
CAGGTGCAGCTACAGCAGTGGGGCGCAGGACTGTTGAAGCCTTCGGAGACCCTGTCCCTCACCTGCGCTGTCTATGGTGG
GTCCTTCAGTGGTTACTACTGGAGCTGGATCCGCCAGCCCCCAGGAAAGGGGCTGGAGTGGATTGGGGAAATCAATCATG
CTGGAAGCACCAACTACAACCCGTCCCTCAAGAGTCGAATCACCATATCAGTGGACACGTCCAAGAACCAGTTCTCCCTG
AAGCTGAGTTCTGTGACCGCCGCGGACACGGCTGTGTATTACTGTGCGAGAGGATGGTACTATGGTTCGGGGAGTTATCA
CCGAAACTGGTTCGACCCCTGGGGCCAGGGAACCCTGGTCACCGTCTCCTCA (SEQ ID NO: 354)
Heavy chain variable region (HCVR, V_{H})-amino acid sequence
QVQLQQWGAGLLKPSETLSLTCAVYGGSFSGYYWSWIRQPPGKGLEWIGEINHAGSTNYNPSLKSRITISVDTSKNQFSL
KLSSVTAADTAVYYCARGWYYGSGSYHRNWFDPWGQGTLVTVSS (SEQ ID NO: 355)
CDR-H1-nucleotide sequence
GGT GGG TCC TTC AGT GGT TAC TAC (SEQ ID NO: 356)
CDR-H1-amino acid sequence
G G S F S G Y Y (SEQ ID NO: 357)
CDR-H2-nucleotide sequence
ATC AAT CAT GCT GGA AGC ACC (SEQ ID NO: 358)
CDR-H2-amino acid sequence
I N H A G S T (SEQ ID NO: 359)
CDR-H3-nucleotide sequence
GCG AGA GGA TGG TAC TAT GGT TCG GGG AGT TAT CAC CGA AAC TGG TTC GAC CCC (SEQ ID
NO: 360)
CDR-H3-amino acid sequence
A R G W Y Y G S G S Y H R N W F D P (SEQ ID NO:
361)
Light chain variable region (LCVR, V_{L})-nucleotide sequence
GAAATTGTATTGACGCAGTCTCCAGGCACCCTGTCTTTGTCTCCAGGGGAAAGAGCCACCCTCTCCTGCAGGGCCAGTCA
GAGTGTTTATTACAGCTACTTAGCCTGGTATCAGCAGAAACCTGGCCAGGCTCCCAGGCTCCTCATCTATGGTGCATCCA
ACAGGGCCACTGGCATCCCAGACAGGTTCAGTGGCAGTGGGTCTGGGACAGACTTCACTCTCACCATCAGCAGACTGGAG
CCTGAAGATTTTGCAGTGTATTACTGTCAGCAGTATGGTAACTCACCTTGGACGTTCGGCCAAGGGACCAAGGTGGAAAT
CAAA (SEQ ID NO: 362)
Light chain variable region (LCVR, V_{L})-amino acid sequence
EIVLTQSPGTLSLSPGERATLSCRASQSVYYSYLAWYQQKPGQAPRLLIYGASNRATGIPDRFSGSGSGTDFTLTISRLE
PEDFAVYYCQQYGNSPWTFGQGTKVEIK (SEQ ID NO: 363)
CDR-L1-nucleotide sequence
CAG AGT GTT TAT TAC AGC TAC (SEQ ID NO: 364)
CDR-L1-amino acid sequence
Q S V Y Y S Y (SEQ ID NO: 365)
CDR-L2-nucleotide sequence
GGT GCA TCC
CDR-L2-amino acid sequence
G A S
CDR-L3-nucleotide sequence
CAG CAG TAT GGT AAC TCA CCT TGG ACG (SEQ ID NO: 368)
CDR-L3-amino acid sequence
Q Q Y G N S P W T (SEQ ID NO: 369)
Heavy chain (HC)-nucleotide sequence
CAGGTGCAGCTACAGCAGTGGGGCGCAGGACTGTTGAAGCCTTCGGAGACCCTGTCCCTCACCTGCGCTGTCTATGGTGG
GTCCTTCAGTGGTTACTACTGGAGCTGGATCCGCCAGCCCCCAGGAAAGGGGCTGGAGTGGATTGGGGAAATCAATCATG
CTGGAAGCACCAACTACAACCCGTCCCTCAAGAGTCGAATCACCATATCAGTGGACACGTCCAAGAACCAGTTCTCCCTG
AAGCTGAGTTCTGTGACCGCCGCGGACACGGCTGTGTATTACTGTGCGAGAGGATGGTACTATGGTTCGGGGAGTTATCA
CCGAAACTGGTTCGACCCCTGGGGCCAGGGAACCCTGGTCACCGTCTCCTCAGCCTCCACCAAGGGCCCATCGGTCTTCC
CCCTGGCGCCCTGCTCCAGGAGCACCTCCGAGAGCACAGCCGCCCTGGGCTGCCTGGTCAAGGACTACTTCCCCGAACCG
GTGACGGTGTCGTGGAACTCAGGCGCCCTGACCAGCGGCGTGCACACCTTCCCGGCTGTCCTACAGTCCTCAGGACTCTA
CTCCCTCAGCAGCGTGGTGACCGTGCCCTCCAGCAGCTTGGGCACGAAGACCTACACCTGCAACGTAGATCACAAGCCCA
GCAACACCAAGGTGGACAAGAGAGTTGAGTCCAAATATGGTCCCCCATGCCCACCCTGCCCAGCACCTGAGTTCCTGGGG
GGACCATCAGTCTTCCTGTTCCCCCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCGTGGAGGTGCATAATGCCAAGACAA
AGCCGCGGGAGGAGCAGTTCAACAGCACGTACCGTGTGGTCAGCGTCCTCACCGTCCTGCACCAGGACTGGCTGAACGGC
AAGGAGTACAAGTGCAAGGTCTCCAACAAAGGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCAAGAACCAGGTCAGCCTGACCTGCCTGGTCA
AAGGCTTCTACCCCAGCGACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTCACCGTGGACAAGAGCAGGTGGCAGGAGGGGAATGTCTT
CTCATGCTCCGTGATGCATGAGGCTCTGCACAACCACTACACACAGAAGTCCCTCTCCCTGTCTCTGGGTAAATGA
(SEQ ID NO: 370)
Heavy chain-amino acid sequence
QVQLQQWGAGLLKPSETLSLTCAVYGGSFSGYYWSWIRQPPGKGLEWIGEINHAGSTNYNPSLKSRITISVDTSKNQFSL
KLSSVTAADTAVYYCARGWYYGSGSYHRNWFDPWGQGTLVTVSSASTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK (SEQ ID NO: 371)
Light chain (LC)-nucleotide sequence
TTACTTCAGGGATCTGGTGAAATTGTATTGACGCAGTCTCCAGGCACCCTGTCTTTGTCTCCAGGGGAAAGAGCCACCCT
CTCCTGCAGGGCCAGTCAGAGTGTTTATTACAGCTACTTAGCCTGGTATCAGCAGAAACCTGGCCAGGCTCCCAGGCTCC
TCATCTATGGTGCATCCAACAGGGCCACTGGCATCCCAGACAGGTTCAGTGGCAGTGGGTCTGGGACAGACTTCACTCTC
ACCATCAGCAGACTGGAGCCTGAAGATTTTGCAGTGTATTACTGTCAGCAGTATGGTAACTCACCTTGGACGTTCGGCCA
AGGGACCAAGGTGGAAATCAAACGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG (SEQ ID NO: 372)
Light chain (LC)-amino acid sequence
LLQGSGEIVLTQSPGTLSLSPGERATLSCRASQSVYYSYLAWYQQKPGQAPRLLIYGASNRATGIPDRFSGSGSGTDFTL
TISRLEPEDFAVYYCQQYGNSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC (SEQ ID NO: 373)
REGN9279
Heavy chain variable region (HCVR, V_{H})-nucleotide sequence
GAAGTGCAGGTGGTGGAGTCTGGGGGAGGCTTGGTACAGCCTGGCAGGTCCCTGAGACTCTCCTGTACAGCCTCTGGATT
CACCTTTGATGATTATGCCATGTTTTGGGTCCGGCAAGGTCCAGGGAAGGGCCTGGAGTGGGTCTCAGGTATTAGTTGGA
ATAGTGGTAGCATAGGCTATGCGGACTCTGTAAAGGGCCGCTTCACCACCTCCAGAGACAACGCCAAGAACTCCCTATAT
TTACAAATGAACAGTCTGAGAACTGAAGACACGGCCTTGTATTACTGTGCAAAAGATTATCGACCCCGTAGTGGGAACCA
CTATAACAACTACGGTATGGACGTCTGGGGCCCAGGGACCACGGTCACCGTCTCCTCA (SEQ ID NO: 374)
Heavy chain variable region (HCVR, V_{H})-amino acid sequence
EVQVVESGGGLVQPGRSLRLSCTASGFTFDDYAMFWVRQGPGKGLEWVSGISWNSGSIGYADSVKGRFTTSRDNAKNSLY
LQMNSLRTEDTALYYCAKDYRPRSGNHYNNYGMDVWGPGTTVTVSS (SEQ ID NO: 375)
CDR-H1-nucleotide sequence
GGA TTC ACC TTT GAT GAT TAT GCC (SEQ ID NO: 376)
CDR-H1-amino acid sequence
G F T F D D Y A (SEQ ID NO: 377)
CDR-H2-nucleotide sequence
ATT AGT TGG AAT AGT GGT AGC ATA (SEQ ID NO: 378)
CDR-H2-amino acid sequence
I S W N S G S I (SEQ ID NO: 379)
CDR-H3-nucleotide sequence
GCA AAA GAT TAT CGA CCC CGT AGT GGG AAC CAC TAT AAC AAC TAC GGT ATG GAC GTC (SEQ
ID NO: 380)
CDR-H3-amino acid sequence
A K D Y R P R S G N H Y N N Y G M D V (SEQ
ID NO: 381)
Light chain variable region (LCVR, V_{L})-nucleotide sequence
GAGATTGTGTTGACGCAGTCTCCAGGCACCCTGTCTTTGTCTCCAGGGGAAAGAGCCACCCTCTCCTGCAGGGCCAGTCA
GAGTTTTCGCGGCAACTACTTAGCCTGGTACCAGCAGAAACCTGGCCAGGCTCCCAGACTCCTCATCTATGGTGCATCCA
GCAGGGCCACTGGCATCCCAGACAGGTTCAGGGGCAGTGGGTCTGGGACAGACTTCACGCTCACCATCAGCAGACTGGAG
CCTGAGGATTTTGCAGTATATTACTGTCACCAGTATGGTAGGTCACCTTGGACGTTCGGCCAAGGGACCAAGGTGGAAAT
CAAA (SEQ ID NO: 382)
Light chain variable region (LCVR, V_{L})-amino acid sequence
EIVLTQSPGTLSLSPGERATLSCRASQSFRGNYLAWYQQKPGQAPRLLIYGASSRATGIPDRFRGSGSGTDFTLTISRLE
PEDFAVYYCHQYGRSPWTFGQGTKVEIK (SEQ ID NO: 383)
CDR-L1-nucleotide sequence
CAG AGT TTT CGC GGC AAC TAC (SEQ ID NO: 384)
CDR-L1-amino acid sequence
Q S F R G N Y (SEQ ID NO: 385)
CDR-L2-nucleotide sequence
GGT GCA TCC
CDR-L2-amino acid sequence
G A S
CDR-L3-nucleotide sequence
CAC CAG TAT GGT AGG TCA CCT TGG ACG (SEQ ID NO: 388)
CDR-L3-amino acid sequence
H Q Y G R S P W T (SEQ ID NO: 389)
Heavy chain (HC)-nucleotide sequence
TTACTTCAGGGATCTGGTGAAGTGCAGGTGGTGGAGTCTGGGGGAGGCTTGGTACAGCCTGGCAGGTCCCTGAGACTCTC
CTGTACAGCCTCTGGATTCACCTTTGATGATTATGCCATGTTTTGGGTCCGGCAAGGTCCAGGGAAGGGCCTGGAGTGGG
TCTCAGGTATTAGTTGGAATAGTGGTAGCATAGGCTATGCGGACTCTGTAAAGGGCCGCTTCACCACCTCCAGAGACAAC
GCCAAGAACTCCCTATATTTACAAATGAACAGTCTGAGAACTGAAGACACGGCCTTGTATTACTGTGCAAAAGATTATCG
ACCCCGTAGTGGGAACCACTATAACAACTACGGTATGGACGTCTGGGGCCCAGGGACCACGGTCACCGTCTCCTCAGCCT
CCACCAAGGGCCCATCGGTCTTCCCCCTGGCGCCCTGCTCCAGGAGCACCTCCGAGAGCACAGCCGCCCTGGGCTGCCTG
GTCAAGGACTACTTCCCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGGCACGAAGACCTACA
CCTGCAACGTAGATCACAAGCCCAGCAACACCAAGGTGGACAAGAGAGTTGAGTCCAAATATGGTCCCCCATGCCCACCC
TGCCCAGCACCTGAGTTCCTGGGGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCAGTTCAACTGGTACGTGGATGGCG
TGGAGGTGCATAATGCCAAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCGTCCTCCATCGAGAAAAC
CATCTCCAAAGCCAAAGGGCAGCCCCGAGAGCCACAGGTGTACACCCTGCCCCCATCCCAGGAGGAGATGACCAAGAACC
AGGTCAGCCTGACCTGCCTGGTCAAAGGCTTCTACCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAGGCTCACCGTGGACAAGAG
CAGGTGGCAGGAGGGGAATGTCTTCTCATGCTCCGTGATGCATGAGGCTCTGCACAACCACTACACACAGAAGTCCCTCT
CCCTGTCTCTGGGTAAATGA (SEQ ID NO: 390)
Heavy chain-amino acid sequence
LLQGSGEVQVVESGGGLVQPGRSLRLSCTASGFTFDDYAMFWVRQGPGKGLEWVSGISWNSGSIGYADSVKGRFTTSRDN
AKNSLYLQMNSLRTEDTALYYCAKDYRPRSGNHYNNYGMDVWGPGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQKSLSLSLGK (SEQ ID NO: 391)
Light chain (LC)-nucleotide sequence
GAGATTGTGTTGACGCAGTCTCCAGGCACCCTGTCTTTGTCTCCAGGGGAAAGAGCCACCCTCTCCTGCAGGGCCAGTCA
GAGTTTTCGCGGCAACTACTTAGCCTGGTACCAGCAGAAACCTGGCCAGGCTCCCAGACTCCTCATCTATGGTGCATCCA
GCAGGGCCACTGGCATCCCAGACAGGTTCAGGGGCAGTGGGTCTGGGACAGACTTCACGCTCACCATCAGCAGACTGGAG
CCTGAGGATTTTGCAGTATATTACTGTCACCAGTATGGTAGGTCACCTTGGACGTTCGGCCAAGGGACCAAGGTGGAAAT
CAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG (SEQ ID NO: 392)
Light chain (LC)-amino acid sequence
EIVLTQSPGTLSLSPGERATLSCRASQSFRGNYLAWYQQKPGQAPRLLIYGASSRATGIPDRFRGSGSGTDFTLTISRLE
PEDFAVYYCHQYGRSPWTFGQGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC (SEQ ID NO: 393)
REGN9280
Heavy chain variable region (HCVR, V_{H})-nucleotide sequence
GAAGTGCAGGTGGTGGAGTCTGGGGGAGGCTTGGTACAGCCTGGCAGGTCCCTGAGACTCTCCTGTACAGCCTCTGGATT
CACCTTTGATGATTATGCCATGTTTTGGGTCCGGCAAGGTCCAGGGAAGGGCCTGGAGTGGGTCTCAGGTATTAGTTGGA
ATAGTGGTAGCATAGGCTATGCGGACTCTGTAAAGGGCCGCTTCACCACCTCCAGAGACAACGCCAAGAACTCCCTATAT
TTACAAATGAACAGTCTGAGAACTGAAGACACGGCCTTGTATTACTGTGCAAAAGATTATCGACCCCGTAGTGGGAACCA
CTATAACAACTACGGTATGGACGTCTGGGGCCCAGGGACCACGGTCACCGTCTCCTCA (SEQ ID NO: 394)
Heavy chain variable region (HCVR, V_{H})-amino acid sequence
EVQVVESGGGLVQPGRSLRLSCTASGFTFDDYAMFWVRQGPGKGLEWVSGISWNSGSIGYADSVKGRFTTSRDNAKNSLY
LQMNSLRTEDTALYYCAKDYRPRSGNHYNNYGMDVWGPGTTVTVSS (SEQ ID NO: 395)
CDR-H1-nucleotide sequence
GGA TTC ACC TTT GAT GAT TAT GCC (SEQ ID NO: 396)
CDR-H1-amino acid sequence
G F T F D D Y A (SEQ ID NO: 397)
CDR-H2-nucleotide sequence
ATT AGT TGG AAT AGT GGT AGC ATA (SEQ ID NO: 398)
CDR-H2-amino acid sequence
I S W N S G S I (SEQ ID NO: 399)
CDR-H3-nucleotide sequence
GCA AAA GAT TAT CGA CCC CGT AGT GGG AAC CAC TAT AAC AAC TAC GGT ATG GAC GTC (SEQ
ID NO: 400)
CDR-H3-amino acid sequence
A K D Y R P R S G N H Y N N Y G M D V (SEQ
ID NO: 401)
Light chain variable region (LCVR, V_{L})-nucleotide sequence
GAGATTGTGTTGACGCAGTCTCCAGGCACCCTGTCTTTGTCTCCAGGGGAAAGAGCCACCCTCTCCTGCAGGGCCAGTCA
GAGTTTTCGCGGCAACTACTTAGCCTGGTACCAGCAGAAACCTGGCCAGGCTCCCAGACTCCTCATCTATGGTGCATCCA
GCAGGGCCACTGGCATCCCAGACAGGTTCAGGGGCAGTGGGTCTGGGACAGACTTCACGCTCACCATCAGCAGACTGGAG
CCTGAGGATTTTGCAGTATATTACTGTCACCAGTATGGTAGGTCACCTTGGACGTTCGGCCAAGGGACCAAGGTGGAAAT
CAAA (SEQ ID NO: 402)
Light chain variable region (LCVR, V_{L})-amino acid sequence
EIVLTQSPGTLSLSPGERATLSCRASQSFRGNYLAWYQQKPGQAPRLLIYGASSRATGIPDRFRGSGSGTDFTLTISRLE
PEDFAVYYCHQYGRSPWTFGQGTKVEIK (SEQ ID NO: 403)
CDR-L1-nucleotide sequence
CAG AGT TTT CGC GGC AAC TAC (SEQ ID NO: 404)
CDR-L1-amino acid sequence
Q S F R G N Y (SEQ ID NO: 405)
CDR-L2-nucleotide sequence
GGT GCA TCC
CDR-L2-amino acid sequence
G A S
CDR-L3-nucleotide sequence
CAC CAG TAT GGT AGG TCA CCT TGG ACG (SEQ ID NO: 408)
CDR-L3-amino acid sequence
H Q Y G R S P W T (SEQ ID NO: 409)
Heavy chain (HC)-nucleotide sequence
GAAGTGCAGGTGGTGGAGTCTGGGGGAGGCTTGGTACAGCCTGGCAGGTCCCTGAGACTCTCCTGTACAGCCTCTGGATT
CACCTTTGATGATTATGCCATGTTTTGGGTCCGGCAAGGTCCAGGGAAGGGCCTGGAGTGGGTCTCAGGTATTAGTTGGA
ATAGTGGTAGCATAGGCTATGCGGACTCTGTAAAGGGCCGCTTCACCACCTCCAGAGACAACGCCAAGAACTCCCTATAT
TTACAAATGAACAGTCTGAGAACTGAAGACACGGCCTTGTATTACTGTGCAAAAGATTATCGACCCCGTAGTGGGAACCA
CTATAACAACTACGGTATGGACGTCTGGGGCCCAGGGACCACGGTCACCGTCTCCTCAGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA (SEQ ID NO: 410)
Heavy chain-amino acid sequence
EVQVVESGGGLVQPGRSLRLSCTASGFTFDDYAMFWVRQGPGKGLEWVSGISWNSGSIGYADSVKGRFTTSRDNAKNSLY
LQMNSLRTEDTALYYCAKDYRPRSGNHYNNYGMDVWGPGTTVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK (SEQ ID NO: 411)
Light chain (LC)-nucleotide sequence
TTACTTCAGGGATCTGGTGAGATTGTGTTGACGCAGTCTCCAGGCACCCTGTCTTTGTCTCCAGGGGAAAGAGCCACCCT
CTCCTGCAGGGCCAGTCAGAGTTTTCGCGGCAACTACTTAGCCTGGTACCAGCAGAAACCTGGCCAGGCTCCCAGACTCC
TCATCTATGGTGCATCCAGCAGGGCCACTGGCATCCCAGACAGGTTCAGGGGCAGTGGGTCTGGGACAGACTTCACGCTC
ACCATCAGCAGACTGGAGCCTGAGGATTTTGCAGTATATTACTGTCACCAGTATGGTAGGTCACCTTGGACGTTCGGCCA
AGGGACCAAGGTGGAAATCAAACGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG (SEQ ID NO: 412)
Light chain (LC)-amino acid sequence
LLQGSGEIVLTQSPGTLSLSPGERATLSCRASQSFRGNYLAWYQQKPGQAPRLLIYGASSRATGIPDRFRGSGSGTDFTL
TISRLEPEDFAVYYCHQYGRSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC (SEQ ID NO: 413)

In some emb2odiments, a heavy chain of an ATDC disclosed herein may optionally lack the C-terminal lysine (K) or glycine-lysine (GK). The C-terminal lysine may contribute to transglutaminase-mediated crosslinking of the antibody to another antibody, resulting in high molecular weight species.

In an embodiment of the invention, an ATDC of the present invention (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) is characterized by one or more of the following:
The anti-GLP1R antibody or antigen-binding fragment without a payload binds GLP1R but is not an agonist antibody or fragment;
Causes a decrease in body weight in obese mice expressing human GLP1R in place of mouse GLP1R administered (e.g., subcutaneously) the ATDC (e.g., about 25 or 100 mg/kg, for example, twice a week for 4 weeks), e.g., by about 3, 7, 14, 28, 38 or 42 days after the first administration; and/or
Causes a decrease in blood glucose in obese mice expressing human GLP1R in place of mouse GLP1R administered (e.g., subcutaneously) the ATDC (e.g., about 25 or 100 mg/kg, for example, twice a week for 4 weeks), e.g., by about 3, 7, 14, 21 and 42 days after the first administration; in an embodiment of the invention, no reduction occurs when about 25 mg/kg of the ATDC are administered.
   “REGN7990”; “REGN9268”; “REGN15869”; “REGN18121”; “REGN18123”; “REGN8070”; “REGN8072”; “REGN9267”; “REGN7988”; “REGN5619”; “REGN7989”; “REGN8069”; “REGN8071”; “REGN9426”; “REGN5203”; “REGN5204”; “REGN5617”; “REGN5619”; “REGN7987”; “REGN9270”; “REGN9278”; “REGN9279”; or “REGN9280” refer to antigen-binding proteins, e.g., antibodies and antigen-binding fragments thereof (including multispecific antigen-binding proteins) that bind specifically to GLP1R, comprising the immunoglobulin heavy chain of SEQ ID NO: 42, 62, 82, 414; 416; 102, 122, 142, 162 or 182 (or variable region thereof) (V_{H}) of SEQ ID NO: 26, 46, 66, 86, 106, 126, 146 or 166 (or a variant thereof), respectively; and the immunoglobulin light chain of SEQ ID NO: 44, 64, 84, 104, 124, 144, 164 or 184 (or variable region thereof) (V_{L}) of SEQ ID NO: 34, 54, 74, 94, 114, 134, 154 or 174 (or a variant thereof), respectively; or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)), e.g., wherein the immunoglobulin chains, variable regions and/or CDRs comprise the specific amino acid sequences described herein. In an embodiment of the invention, the V_{H} is linked to an IgG constant heavy chain domain (e.g., IgG1 or IgG4) and/or the V_{L} is linked to a lambda or kappa constant light chain domain. “REGN7990”; “REGN9268”; “REGN15869”; “REGN18121”; “REGN18123”; “REGN8070”; “REGN8072”; “REGN9267”; “REGN7988”; “REGN5619”; “REGN7989”; “REGN8069”; “REGN8071”; “REGN9426”; “REGN5203”; “REGN5204”; “REGN5617”; “REGN5619”; “REGN7987”; “REGN9270”; “REGN9278”; “REGN9279”; or “REGN9280” also include such antibodies or antigen-binding fragments having the immunoglobulins discussed herein but lacking the N-terminal sequence LLQGSG (SEQ ID NO: 18) on the light chain. “REGN7990”; “REGN9268”; “REGN15869”; “REGN18121”; “REGN18123”; “REGN8070”; “REGN8072”; “REGN9267”; “REGN7988”; “REGN5619”; “REGN7989”; “REGN8069”; “REGN8071”; “REGN9426”; “REGN5203”; “REGN5204”; “REGN5617”; “REGN5619”; “REGN7987”; “REGN9270”; “REGN9278”; “REGN9279”; or “REGN9280” also include antibody tethered drug conjugates including the immunoglobulins discussed herein tethered, e.g., by a linker, to a drug payload such as a GLP1 peptidomimetic, e.g., LP11, LP30 or LP32. “REGN7990”; “REGN9268”; “REGN15869”; “REGN18121”; “REGN18123”; “REGN8070”; “REGN8072”; “REGN9267”; “REGN7988”; “REGN5619”; “REGN7989”; “REGN8069”; “REGN8071”; “REGN9426”; “REGN5203”; “REGN5204”; “REGN5617”; “REGN5619”; “REGN7987”; “REGN9270”; “REGN9278”; “REGN9279”; or “REGN9280” having a given payload or linker-payload, e.g., LP11, LP30 or LP32 (e.g., ATDCs), may be named specifically as, for example, “REGN7990-LP30”, “REGN9268-LP32” or “REGN15869-LP11”.

In one aspect, the present disclosure provides antibodies and antigen-binding fragments that bind specifically to GLP1R, conjugated to one or more GLP1 peptidomimetics via non-cleavable linker. Illustrative non-limiting examples of ATDCs of the present invention include Formula (I) or (A) described herein.

In some embodiments of the invention, the non-cleavable linker in an ATDC of the present disclosure is stable after the ATDC is administered into the body, e.g., a human body. For example, the non-cleavable linker can be stable in plasma, e.g., in human plasma, stable upon binding cell surface, or stable upon antibody binding its target antigen and/or GLP1 peptidomimetic binding GLP1R. In some embodiments, the non-cleavable linker is more stable in vivo than either the payload or the antibody under the same physiological conditions. In some embodiments of the invention, an ATDC may be degraded in the lysosome to release the payload, the linker-payload, and/or its ATDC metabolites/catabolites, which in certain embodiments are effective for GLP1R activation either locally or systemically.

In some embodiments, the ATDC is stable in plasma and degrades in the lysosome. In some embodiments, the ATDC is stable in plasma and does not degrade in the lysosome.

The present invention provides an ATDC that comprises the structure of Formula (A):

BA-(L-P)ₘ (A),

wherein:
BA is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280;
L is a non-cleavable linker;
P is a payload having the structure selected from the group consisting of (SEQ ID NOS 448-450, respectively, in order of appearance):

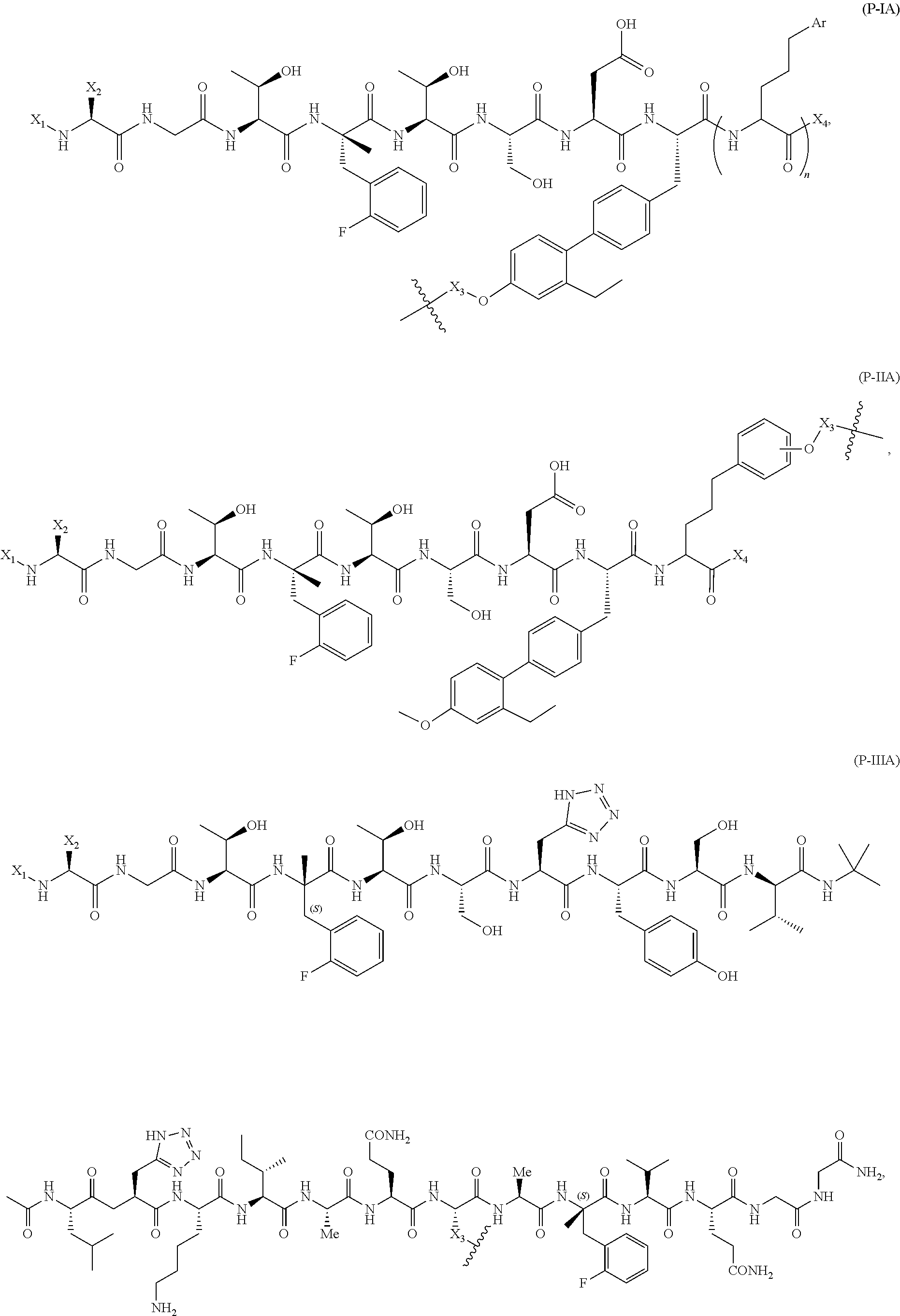

wherein

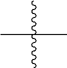

is the point of attachment of the payload to L;
X₁ is selected from H;

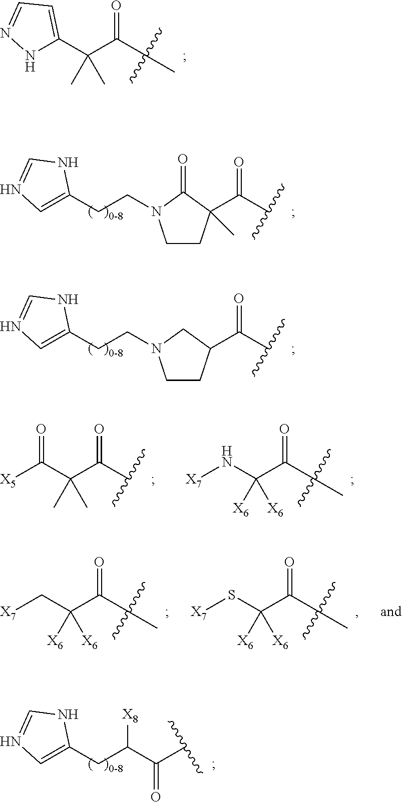

X₂ is selected from

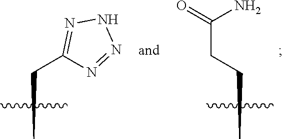

X₃ is selected from a bond, —(CH₂)₂₋₆—NH—, —(CH₂)₂₋₆-Tr-, and —(CH₂)₂₋₆-Tr-(CH₂)₁₋₆—NH, where Tr is a triazole moiety;
n is 0 or 1;
X₄ is selected from —NH₂, —OH and
X₅ is selected from —OH, —NH₂, —NH—OH, and

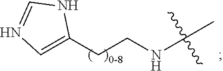

X₆ is independently at each occurrence selected from H, —OH, —CH₃, and —CH₂OH;
X₇ is selected from H,

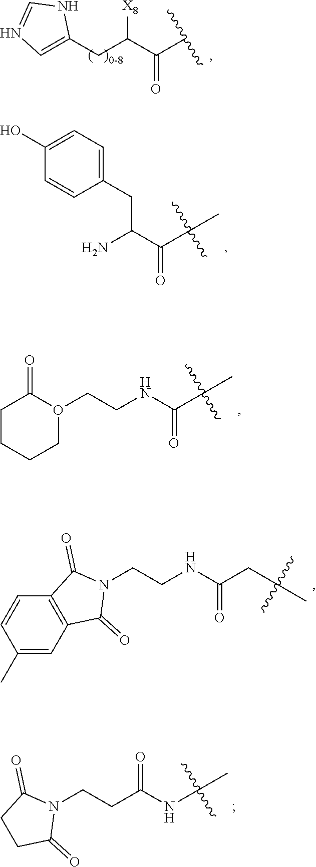

X₈ is selected from H, —OH, —NH₂, and

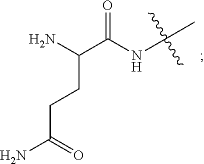 [SMILES: *NC(=O)C(N)CCC(N)=O]

Ar is selected from

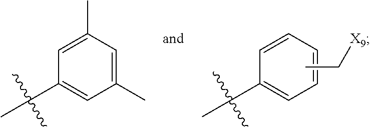

X₉ is selected from —NH₂

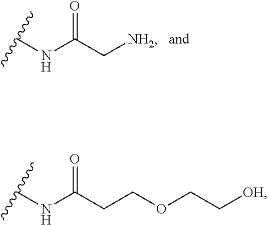 [SMILES: *NC(=O)CN ; *NC(=O)CCOCCO]

and
m is an integer from 1 to 4
or a pharmaceutically acceptable salt thereof.

In one embodiment, m is 1. In one embodiment, m is an integer from 2 to 4. In one embodiment, m is 2.

The present invention provides an ATDC that comprises the structure of Formula (I):

BA-L-P (I),

wherein:
BA is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280;
L is a non-cleavable linker;
P is a payload having the structure selected from the group consisting of (SEQ ID NOS 451-452, respectively, in order of appearance):

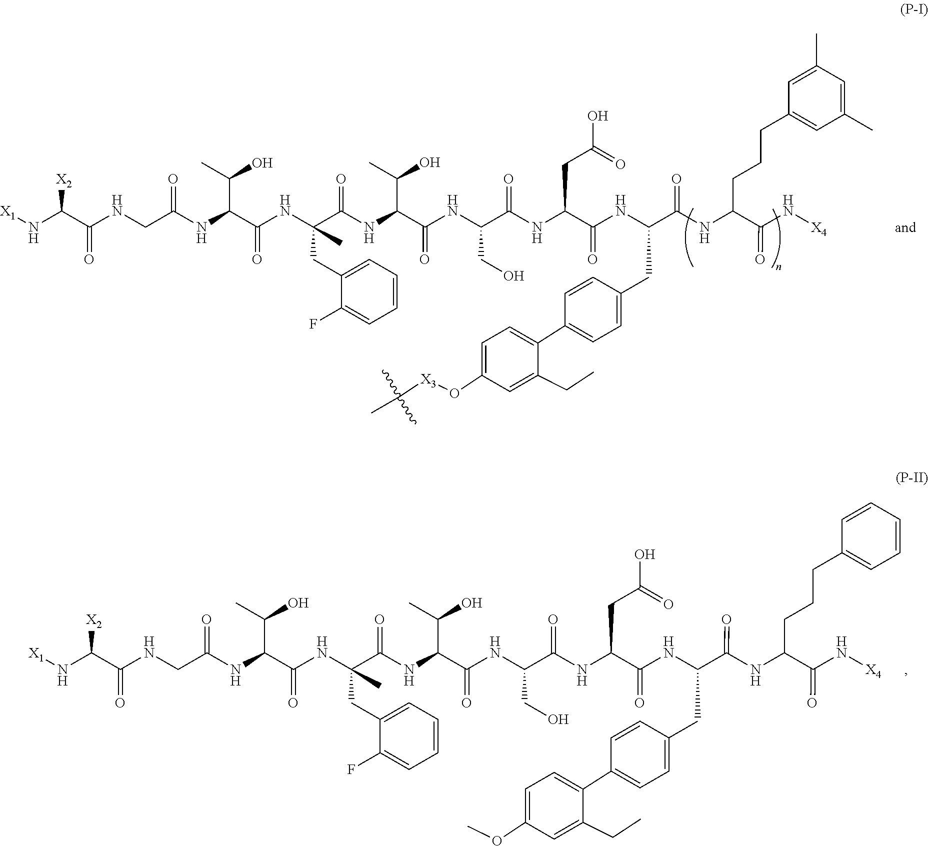

wherein

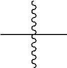

is the point of attachment of the payload to L;
X₁ is selected from H;

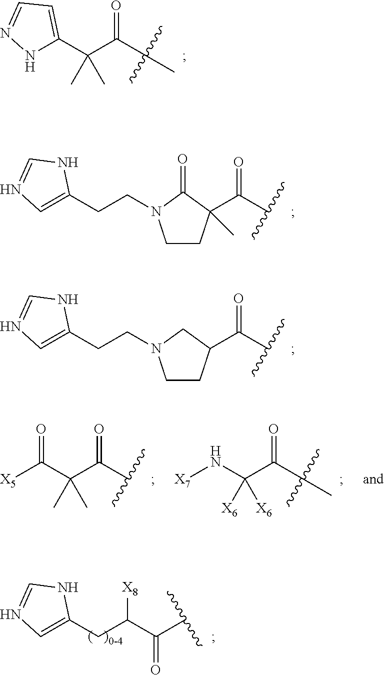

X₂ is selected from

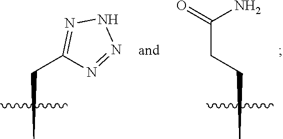

X₃ is selected from —(CH₂)₂₋₆—NH— and —(CH₂)₂₋₆-Tr-, where Tr is a triazole moiety;
n is 0 or 1;
X₄ is selected from H and phenyl;
X₅ is selected from —OH, —NH₂, —NH—OH, and

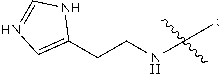 [SMILES: *NCCc1cnc[nH]1]

X₆ is independently at each occurrence selected from H, —OH, —CH₃, and —CH₂OH;
X₇ is selected from H,

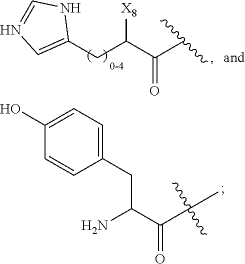

X₈ is selected from H, —OH, —NH₂, and

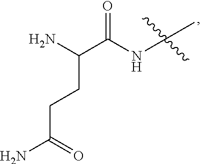 [SMILES: *NC(=O)C(N)CCC(N)=O]

or a pharmaceutically acceptable salt thereof.

In one embodiment of the invention, the linker L is a non-cleavable linker, i.e., a linker which is stable and provides a covalent connection between the antibody or antigen-binding fragment (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) and the drug, e.g., between an anti-GLP1R antibody or fragment and a GLP1 peptidomimetic payload P according to the present disclosure. In some embodiments of the invention, the non-cleavable linker L of the present disclosure is stable after the ATDC is administered into the body, e.g., a human body. For example, the linker L can be stable in plasma, e.g., in human plasma, stable upon binding cell surface, or stable upon antibody binding its target antigen and/or GLP1 peptidomimetic binding GLP1R. In some embodiments of the invention, the linker L is more stable in vivo than either the payload or the antibody under the same physiological conditions.

In one embodiment, the linker L has the structure of formula (L′):

—La—Y-Lp- (L′),

wherein La is a first linker covalently attached to the antibody or an antigen-binding fragment thereof;
Y is a group comprising a triazole, a Diels-Alder adduct, or a thiol-maleimide adduct, and
Lp is absent or a second linker covalently attached to the payload P according to the present disclosure, wherein when Lp is absent Y is also absent.

In one embodiment, Y is a group comprising a triazole.

In another embodiment, Y is a group comprising a Diels-Alder adduct.

In one embodiment, the linker L has the structure of formula (L′):

—La—Y-Lp- (L′),

wherein La is a first linker covalently attached to the antibody or an antigen-binding fragment thereof;
Y is a group comprising a triazole, and
Lp is absent or a second linker covalently attached to the payload P according to the present disclosure.

In one embodiment, La comprises C₁₋₆ alkyl, phenyl, —NH—, —C(O)—, —(CH₂)ᵤ—NH—C(O)—, —(CH₂)ᵤ—C(O)—NH—, —(CH₂—CH₂—O)ᵤ—, —(CH₂)ᵤ—(O—CH₂—CH₂)ᵥ—C(O)—NH—, a peptide unit comprising from 2 to 4 amino acids, or combinations thereof, each of which may be optionally substituted with one or more of —S—, —S(O₂)—, —C(O)—, —C(O₂)—; and CO₂H, wherein subscripts u and v are independently an integer from 1 to 8.

In one embodiment, La is selected from the group consisting of:

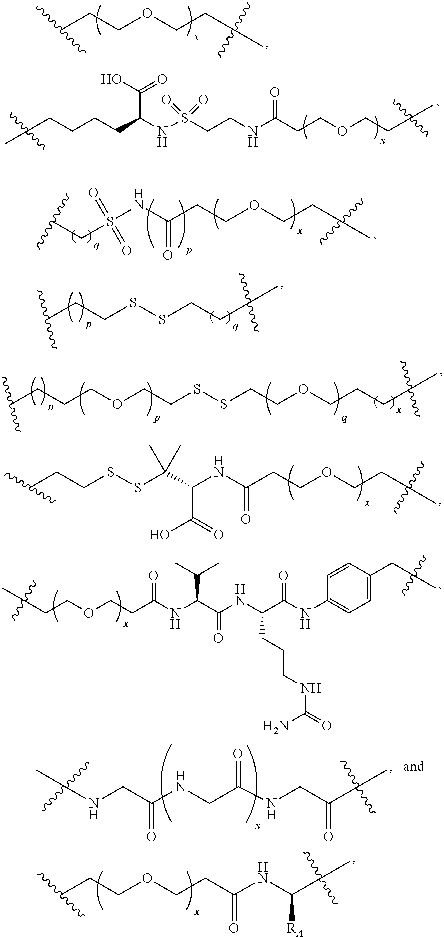

wherein R_{A} is a group comprising an alkyne, an azide, a tetrazine, a trans-cyclooctene, a maleimide, an amine, a ketone, an aldehyde, a carboxylic acid, an ester, a thiol, a sulfonic acid, a tosylate, a halide, a silane, a cyano group, a carbohydrate group, a biotin group, a lipid residue and wherein subscripts x, n, p and q are independently an integer from 0 to 12, and combinations thereof.

In one embodiment, —La— is selected from the group consisting of:

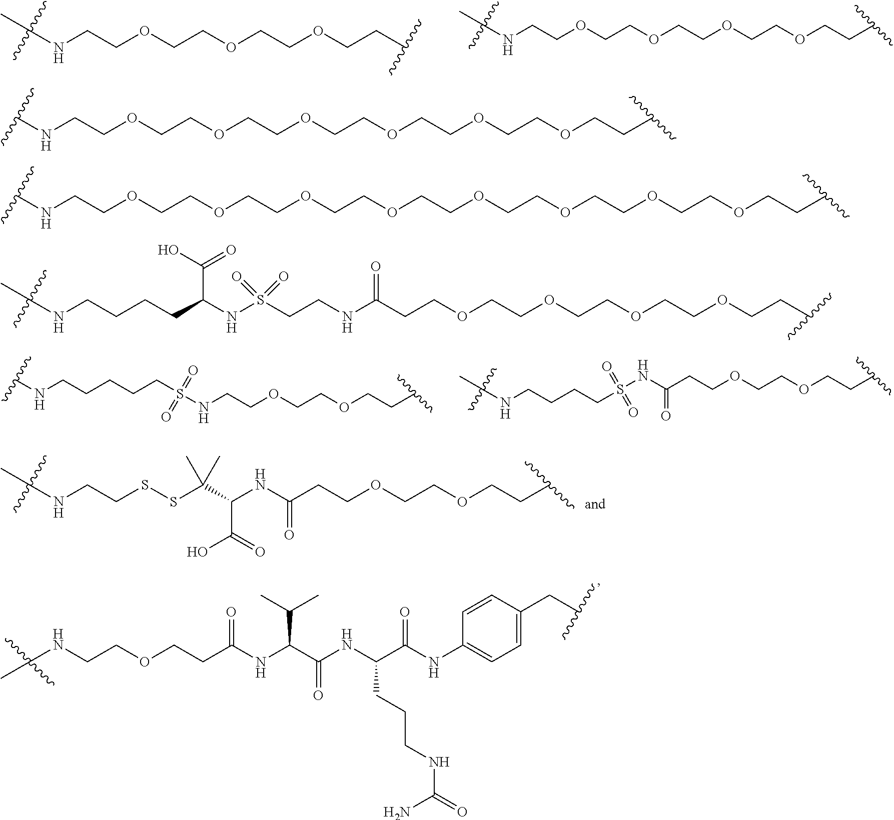

where the

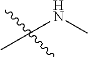 [SMILES: *NC]

is the amino point of attachment to a residue (eg., a glutamine residue) of the antibody and/or the antigen-containing fragment thereof.

In one embodiment, —La— is

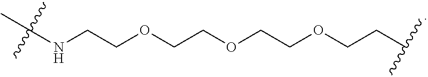 [SMILES: *CCOCCOCCOCCN*]

In another embodiment, —La— is selected from the group consisting of:

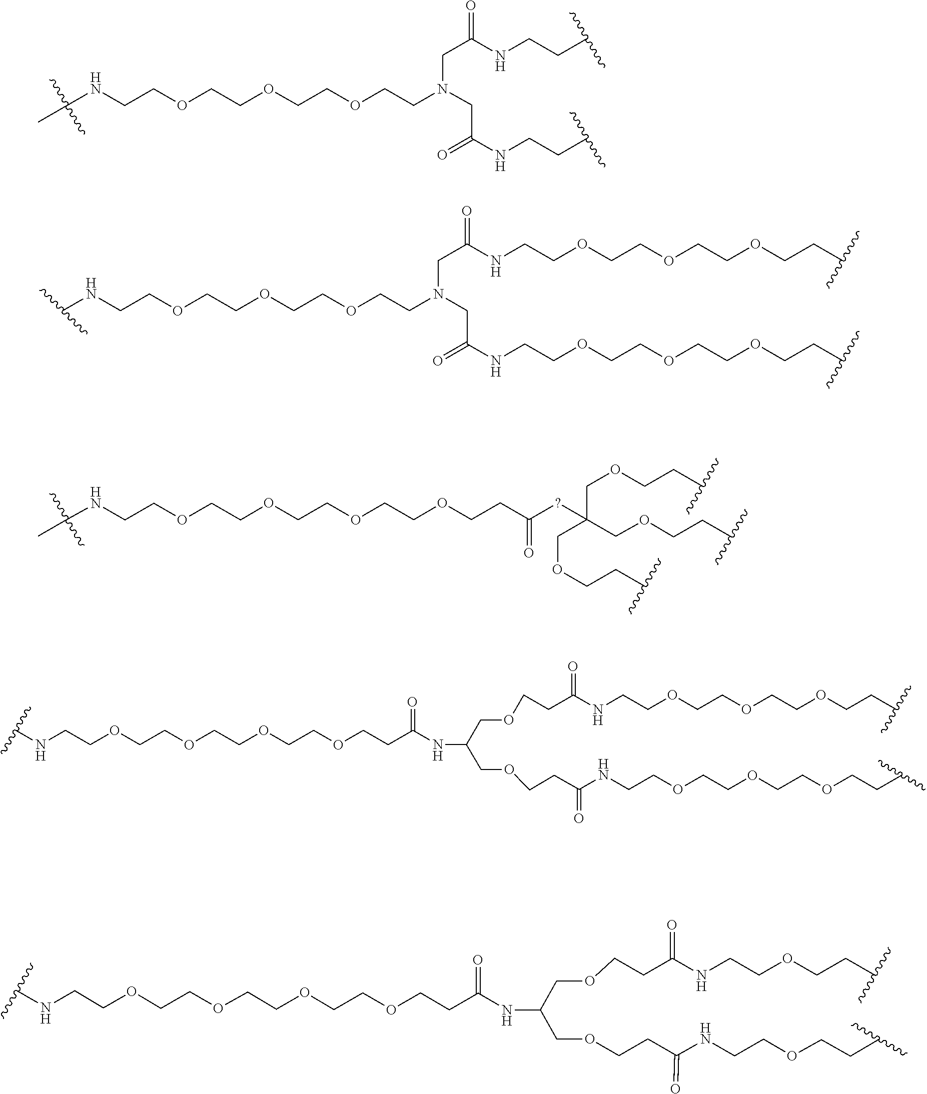

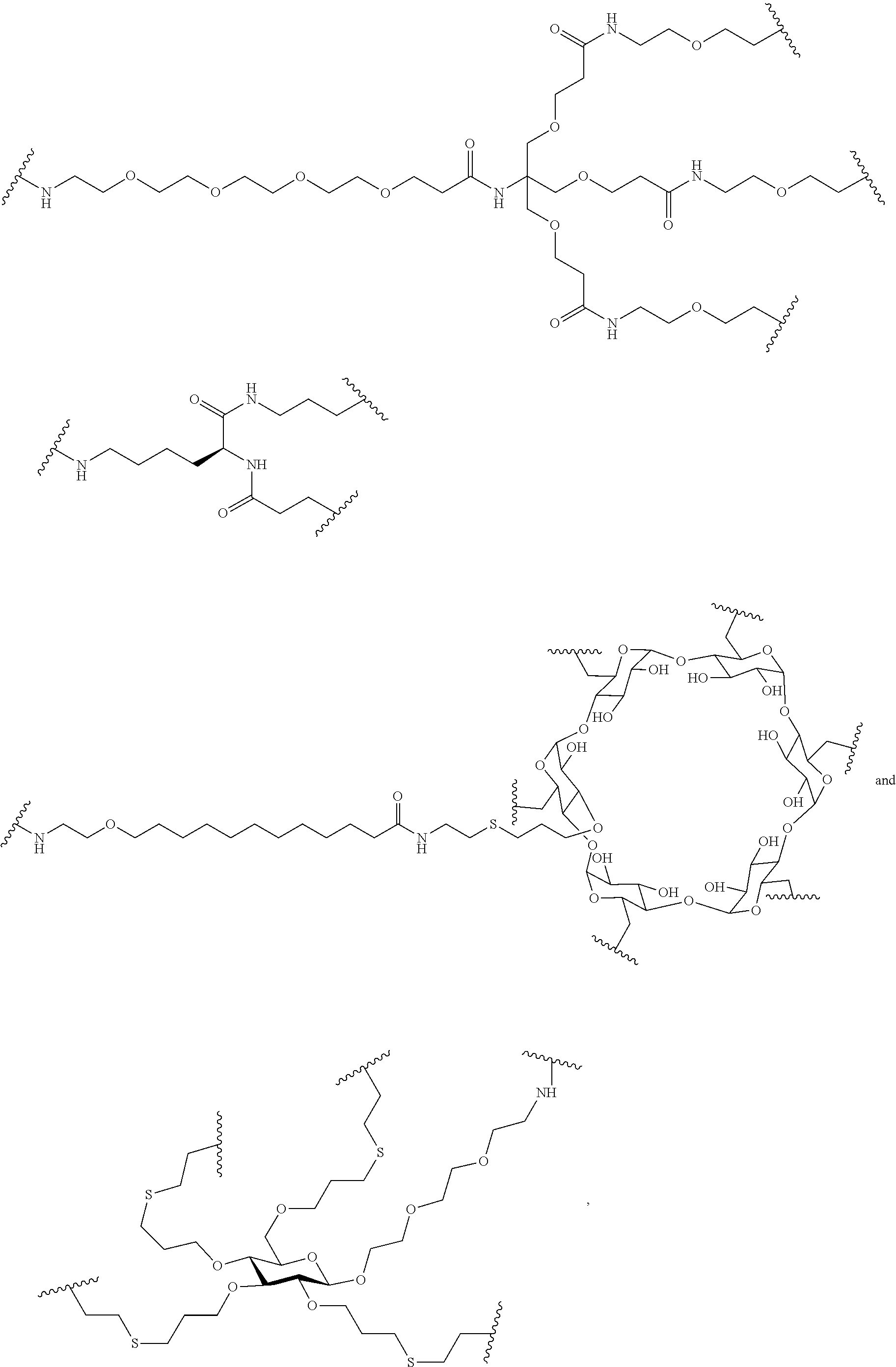

where the

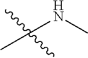 [SMILES: *NC]

is the amino point of attachment to a residue (e.g., a glutamine residue) of the antibody and/or the antigen-containing fragment thereof.

In some embodiments of the invention, La comprises a polyethylene glycol (PEG) segment having 1 to 36 —CH₂CH₂O— (EG) units. In some embodiments of the invention, the PEG segment comprises 4 EG units, or 8 EG units, or 12 EG units, or 24 EG units. In some embodiments of the invention, the PEG segment comprises 8 EG units. In some embodiments, La has a structure selected from the group consisting of

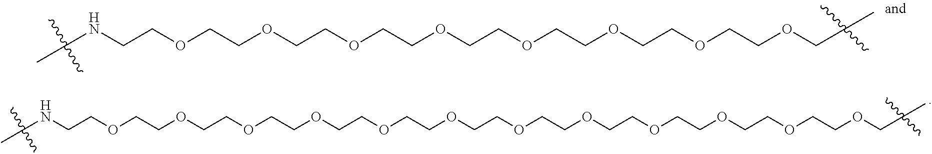 [SMILES: *COCCOCCOCCOCCOCCOCCOCCOCCN* ; *COCCOCCOCCOCCOCCOCCOCCOCCOCCOCCOCCOCCN*]

In some embodiments of the invention, La comprises one or more amino acids selected from glycine, threonine, serine, glutamine, glutamic acid, alanine, valine, leucine, and proline and combinations thereof. In some embodiments of the invention, La comprises 1 to 10 glycines and 1 to 6 serines. In some embodiments of the invention, La comprises 4 glycines and 1 serine. In some embodiments of the invention, La is selected from the group consisting of Gly-Gly-Gly-Gly-Ser (G₄S) (SEQ ID NO: 1), Ser-Gly-Gly-Gly-Gly (SG₄) (SEQ ID NO: 2), Gly-Gly-Ser-Gly-Gly-Ser-Gly-Gly (G₂S-G₂S-G₂) (SEQ ID NO: 438), and Gly-Gly-Gly-Gly-Ser-Gly-Gly-Gly-Gly (G₄S-G₄) (SEQ ID NO: 419).

In some embodiments of the invention, La comprises a combination of a PEG segment having 1 to 36 EG units and one or more amino acids selected from glycine, threonine, serine, glutamine, glutamic acid, alanine, valine, leucine, and proline and combinations thereof. In some embodiments of the invention, La is selected from the group consisting of (SEQ ID NOS 459-460, respectively, in order of appearance):

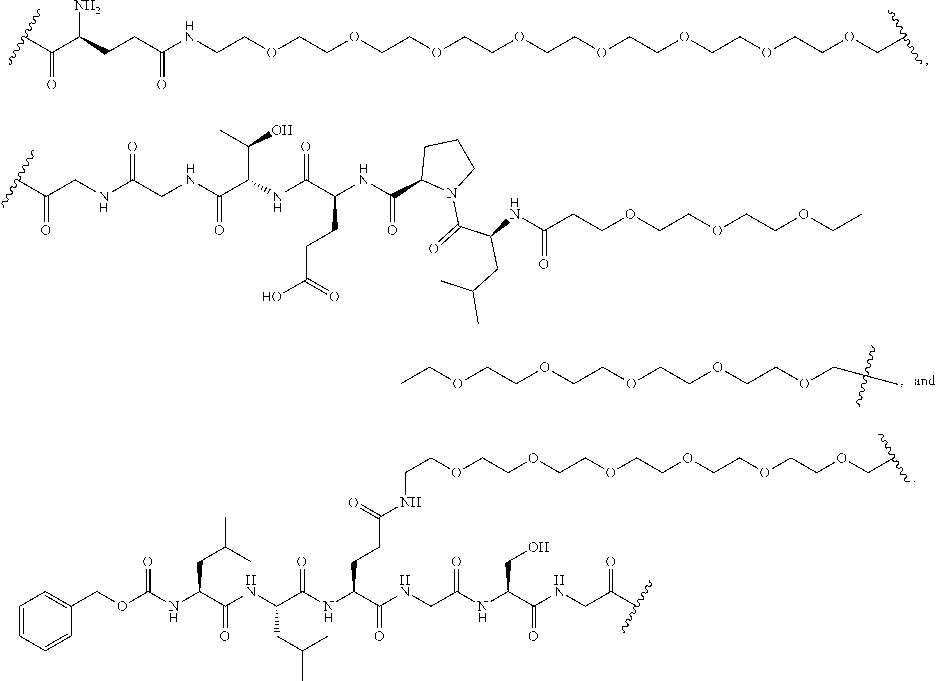

In some embodiments, La comprises a —(CH₂)₂₋₂₄— chain. In some embodiments of the invention, La comprises a combination of a —(CH₂)₂₋₂₄— chain, a PEG segment having 1 to 36 EG units and one or more amino acids selected from glycine, threonine, serine, glutamine, glutamic acid, alanine, valine, leucine, and proline and combinations thereof. La is selected from the group consisting of (SEQ ID NOS 609 and 533, respectively, in order of appearance):

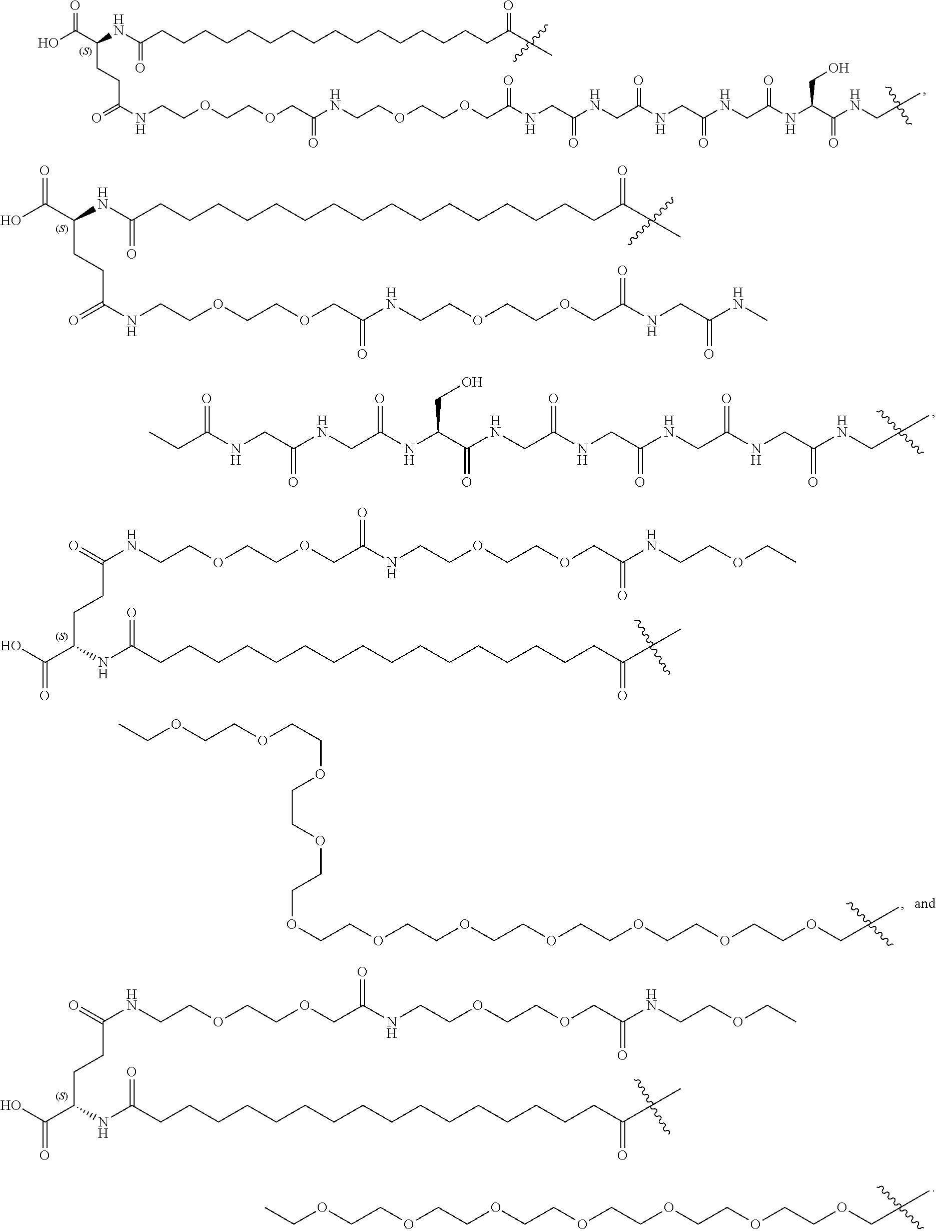

wherein Q is C or N.

In one embodiment of the invention, Y has a structure selected from the group consisting of:

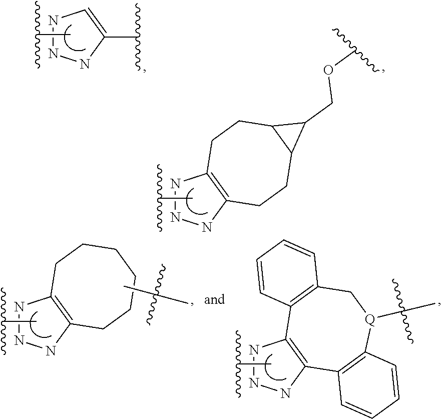

In one embodiment of the invention, the linker L, or the first linker La, or the second linker Lp, comprises a polyethylene glycol (PEG) segment having 1 to 36 —CH₂CH₂O— (EG) units.

In one embodiment of the invention, the PEG segment comprises between 2 and 30 EG units, or between 4 and 24 EG units. In one embodiment, the PEG segment comprises 2 EG units, or 4 EG units, or 6 EG units, or 8 EG units, or 10 EG units, or 12 EG units, or 14 EG units, or 16 EG units, or 18 EG units, or 20 EG units, or 22 EG units, or 24 EG units.

In one embodiment of the invention, the PEG segment comprises 4 EG units. In one embodiment, the PEG segment comprises 8 EG units. In one embodiment, the PEG segment comprises 12 EG units. In one embodiment, the PEG segment comprises 24 EG units.

In one embodiment of the invention, the PEG segment comprises 4 to 8 EG units. In one embodiment, the PEG segment comprises 4 EG units or 8 EG units.

In one embodiment of the invention, La comprises a PEG segment having 3 EG units.

In one embodiment of the invention, Lp comprises a PEG segment having 4 EG units. In one embodiment, Lp comprises a PEG segment having 8 EG units.

In one embodiment of the invention, the Y-Lp has a structure selected from the group consisting of:

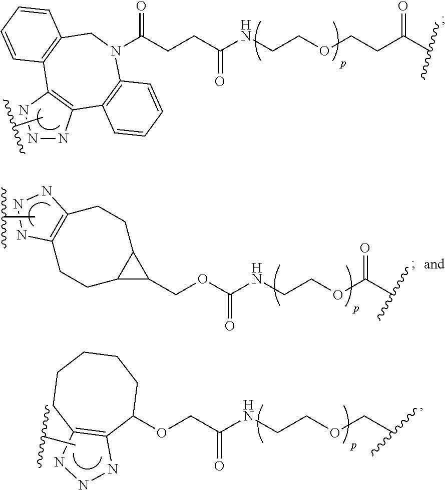

wherein p is an integer from 1 to 36.

In one embodiment of the invention, the Y-Lp has a structure selected from the group consisting of:

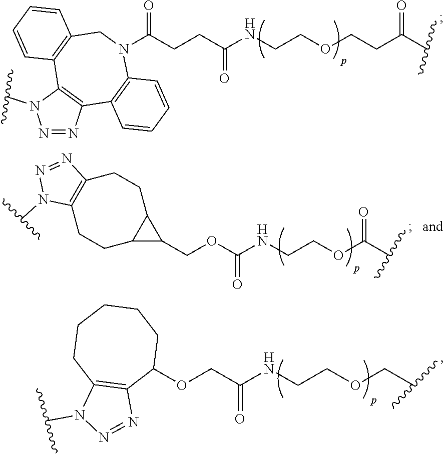

or a triazole regioisomer thereof,
wherein p is an integer from 1 to 36.

In another embodiment of the invention, the linker L or the first linker La, or the second linker Lp, comprises one or more amino acids selected from glycine, serine, glutamic acid, alanine, valine, and proline and combinations thereof.

In one embodiment of the invention, the linker L or the first linker La, or the second linker Lp, comprises from 1 to 10 glycines, or 1 glycine, or 2 glycines, or 3 glycines, or 4 glycines, or 5 glycines, or 6 glycines, or 7 glycines, or 8 glycines, or 9 glycines, or 10 glycines.

In one embodiment of the invention, the linker L or the first linker La, or the second linker Lp, comprises from 1 to 6 serines, or 1 serine, or 2 serines, or 3 serines, or 4 serines, or 5 serines, or 6 serines.

In one embodiment of the invention, the linker L or the first linker La, or the second linker Lp, comprises 1 to 10 glycines and 1 to 6 serines.

In one embodiment of the invention, the linker L or the first linker La, or the second linker Lp, comprises 4 glycines and 1 serine.

In one embodiment of the invention, the linker L or the first linker La, or the second linker Lp, is selected from the group consisting of Gly-Gly-Gly-Gly-Ser (G4S) (SEQ ID NO: 1), Ser-Gly-Gly-Gly-Gly (SG4) (SEQ ID NO: 2), and Gly-Gly-Gly-Gly-Ser-Gly-Gly-Gly-Gly-Ser (G4S-G4S) (SEQ ID NO: 3).

In some embodiments of the invention, one or more serine residues comprise a carbohydrate group, e.g., a glucose group.

In one embodiment of the invention, the linker L or the first linker La, or the second linker Lp, comprises from 1 to 10 glutamic acids and from 1 to 10 glycines.

In some embodiments of the invention, the linker L or the first linker La, or the second linker Lp, comprises a combination of a polyethylene glycol (PEG) segment having 1 to 36 —CH₂CH₂O— (EG) units and one or more amino acids selected from glycine, serine, glutamic acid, alanine, valine, and proline and combinations thereof.

In one embodiment of the invention, the linker L or the first linker La, or the second linker Lp, comprises a combination of a PEG segment having 1 to 36 EG units and 1 to 10 glycines. In one embodiment of the invention, the linker L or the first linker La, or the second linker Lp, comprises a combination of a PEG segment having 1 to 36 EG units and a group selected from Gly-Gly-Gly-Gly-Ser (G4S) (SEQ ID NO: 1), Ser-Gly-Gly-Gly-Gly (SG4) (SEQ ID NO: 2), and Gly-Gly-Gly-Gly-Ser-Gly-Gly-Gly-Gly-Ser (G4S-G4S) (SEQ ID NO: 3).

In one embodiment of the invention, the linker L or the first linker La, or the second linker Lp, has a structure selected from the group consisting of (SEQ ID NOS 567-568, respectively, in order of appearance):

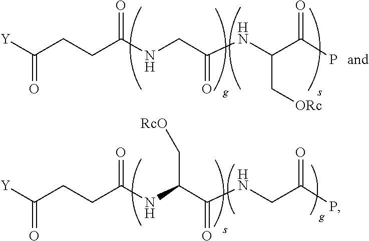

wherein Y is the group comprising a triazole, e.g., as shown above, and P is the payload, and wherein Rc is selected from hydrogen (H) and glucose, g is an integer from 1 to 10 and s is an integer from 0 to 4.

In one embodiment of the invention, the Y-Lp has a structure selected from the group consisting of (SEQ ID NOS 453-458, respectively, in order of appearance):

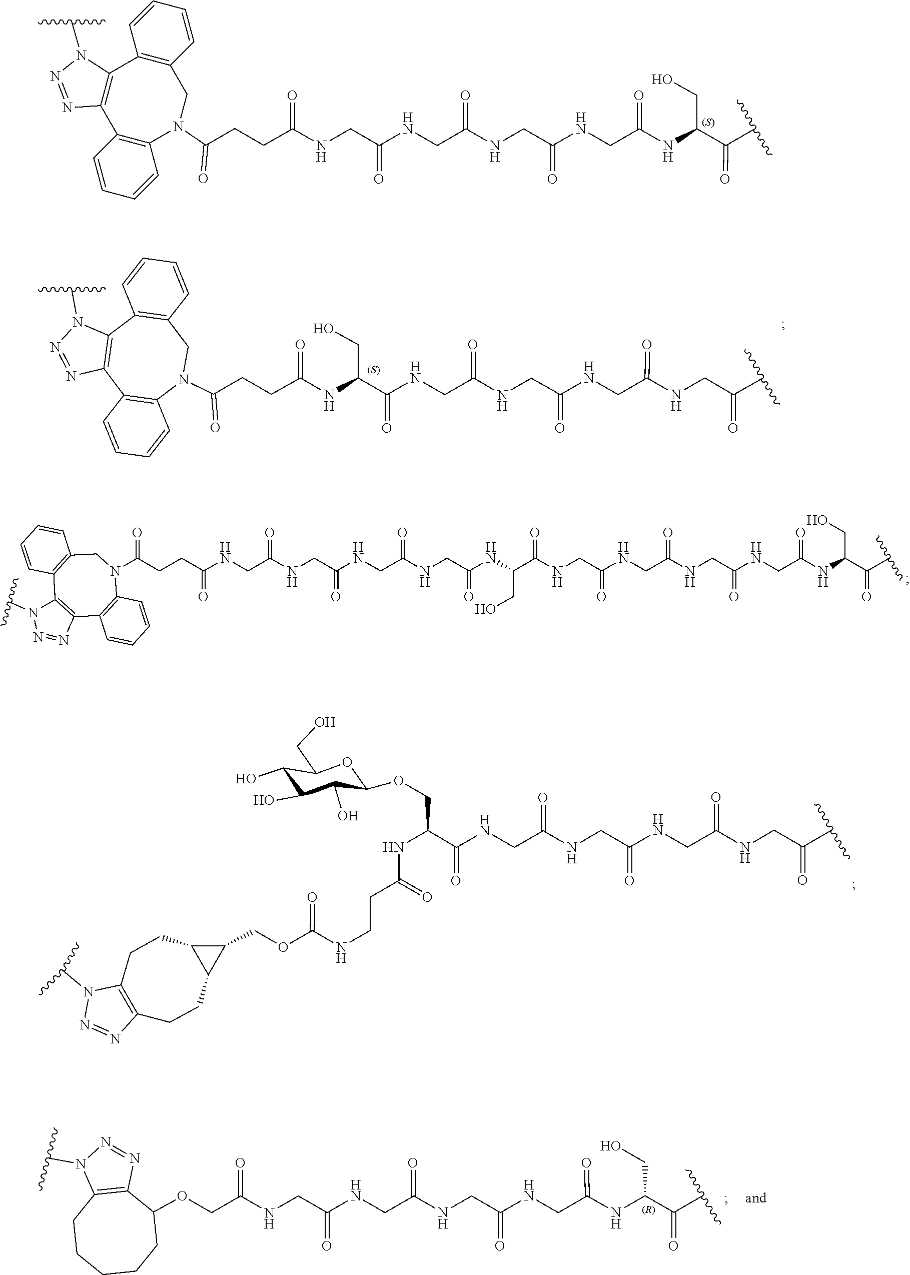

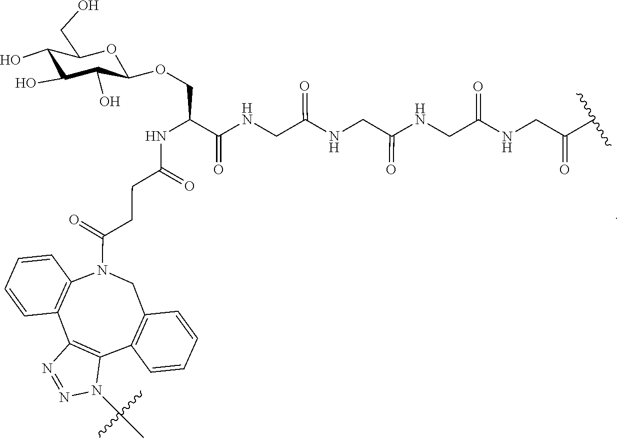

In one embodiment of the invention, the linker L comprises a cyclodextrin moiety.

In some embodiments of the invention, the linker L is attached to the antibody or an antigen-binding fragment thereof (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) via a glutamine residue. In some embodiments of the invention, the linker L is attached to the antibody or an antigen-binding fragment thereof via a lysine residue. In some embodiments of the invention, the linker L is attached to the antibody or an antigen-binding fragment thereof via a cysteine residue.

In one aspect of the invention, the payloads P according to the present disclosure have a structure of Formula selected from the group consisting of Formula (P-IB) (SEQ ID NO: 508), Formula (P-IIB) (SEQ ID NO: 509), and Formula (P-IIIB) (SEQ ID NO: 510):

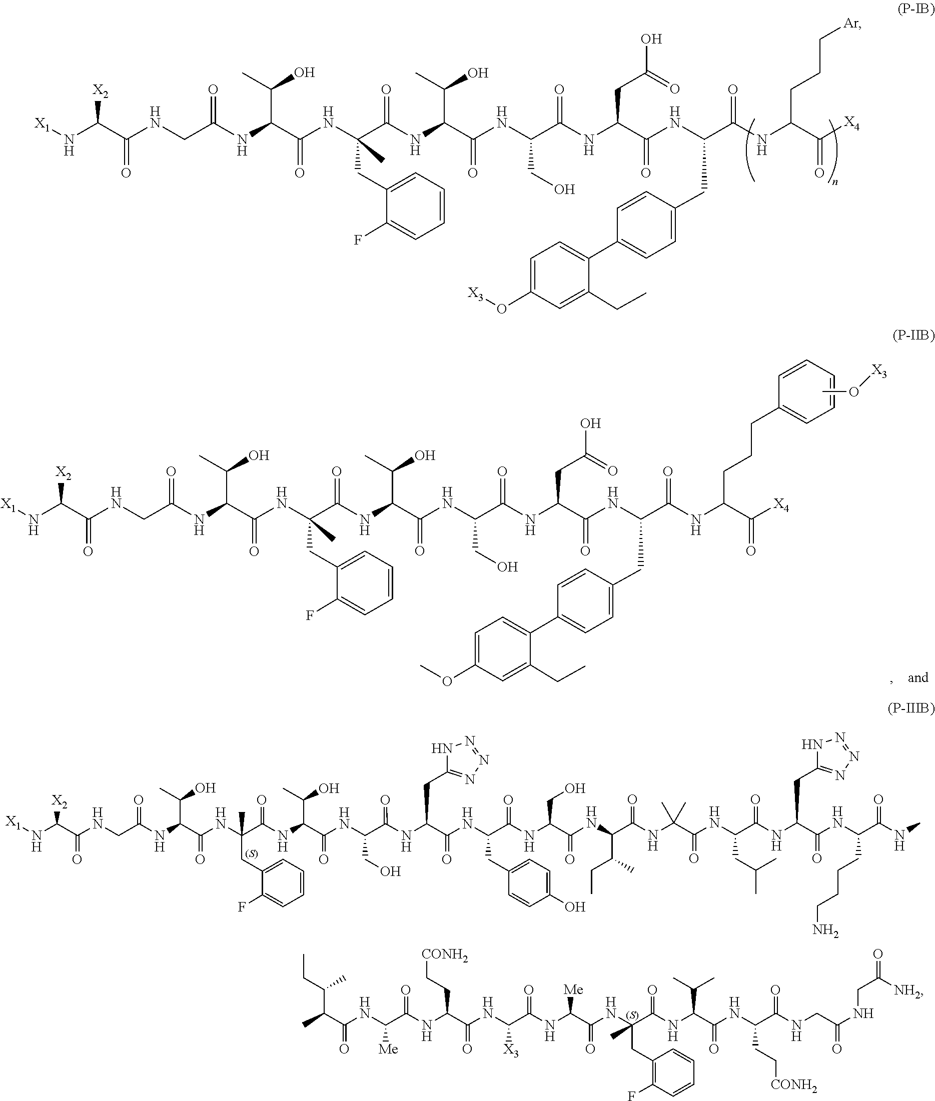

wherein:
X₁ is selected from H;

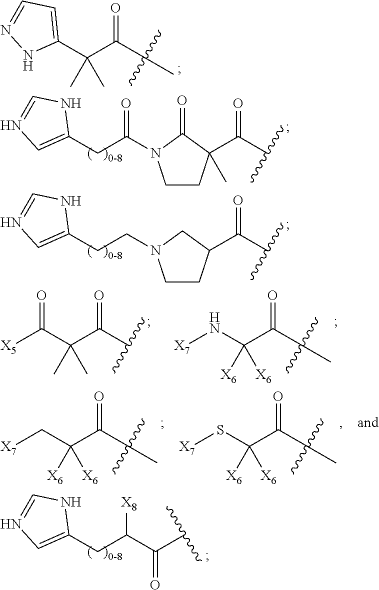

X₂ is selected from

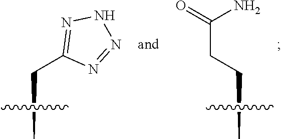

X₃ is selected from —CH₃, —(CH₂)₂₋₆—NH₂, —(CH₂)₂₋₆—N₃, and —(CH₂)₂₋₆-Tr-(CH₂)₁₋₆—NH₂, where Tr is a triazole moiety;
n is 0 or 1;
X₄ is selected from —NH₂, —OH and —N(H)(phenyl);
X₅ is selected from —OH, —NH₂, —NH—OH, and

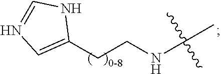

X₆ is independently at each occurrence selected from H, —OH, —CH₃, and —CH₂OH;
X₇ is selected from H,

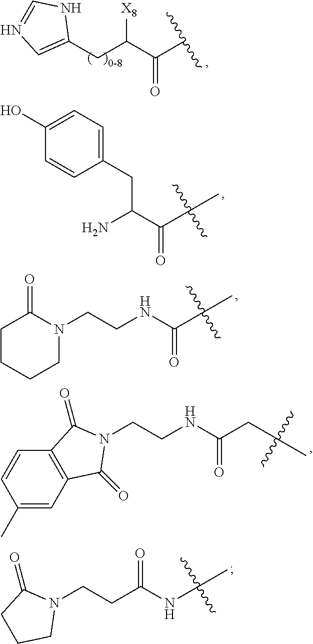

X₈ is selected from H, —OH, —NH₂, and

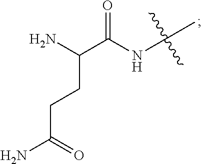 [SMILES: *NC(=O)C(N)CCC(N)=O]

Ar is selected from

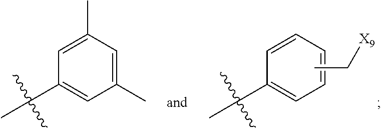

X₉ is selected from —NH₂,

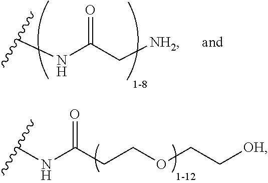

and
m is an integer from 1 to 4,
   or a pharmaceutically acceptable salt thereof.

In one embodiment of the invention, the payloads P according to the present disclosure have a structure of Formula (II) (SEQ ID NO: 511):

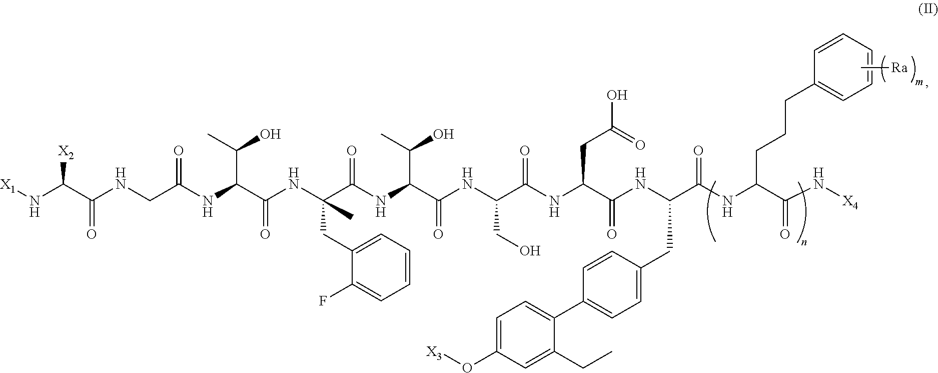

wherein:
X₁ is selected from H;

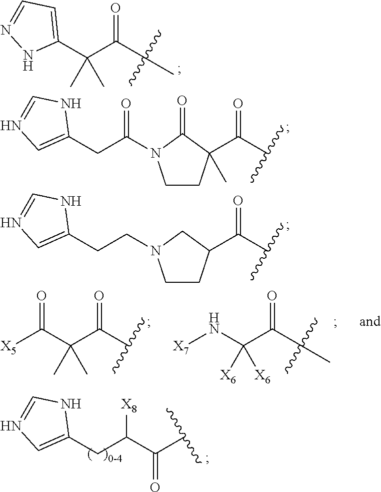

X₂ is selected from

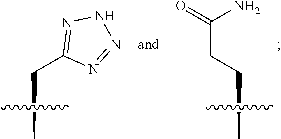

X₃ is selected from —(CH₂)₂₋₆—NH₂, —(CH₂)₂₋₆—N₃, and —CH₃, with the proviso that when X₃ is —CH₃, n is 1 and Ra in at least one occurrence is selected from —(CH₂)₂₋₆—NH₂ and —(CH₂)₂₋₆—N₃;
n is 0 or 1;
m is an integer from 0 to 3;
Ra is independently at each occurrence selected from —CH₃, —(CH₂)₂₋₆—NH₂, and —(CH₂)₂₋₆—N₃;
X₄ is selected from H and phenyl;
X₅ is selected from —OH, —NH₂, —NH—OH, and

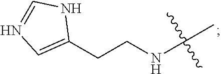 [SMILES: *NCCc1cnc[nH]1]

X₆ is independently at each occurrence selected from H, —OH, —CH₃, and —CH₂OH;
X₇ is selected from H,

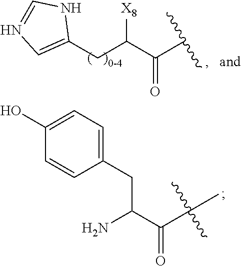

X₈ is selected from H, —OH, —NH₂, and

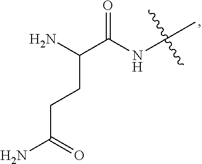 [SMILES: *NC(=O)C(N)CCC(N)=O]

and pharmaceutically acceptable salts thereof.

In one embodiment of the invention, the payload P has a structure selected from (SEQ ID NOS 451-452, respectively, in order of appearance):

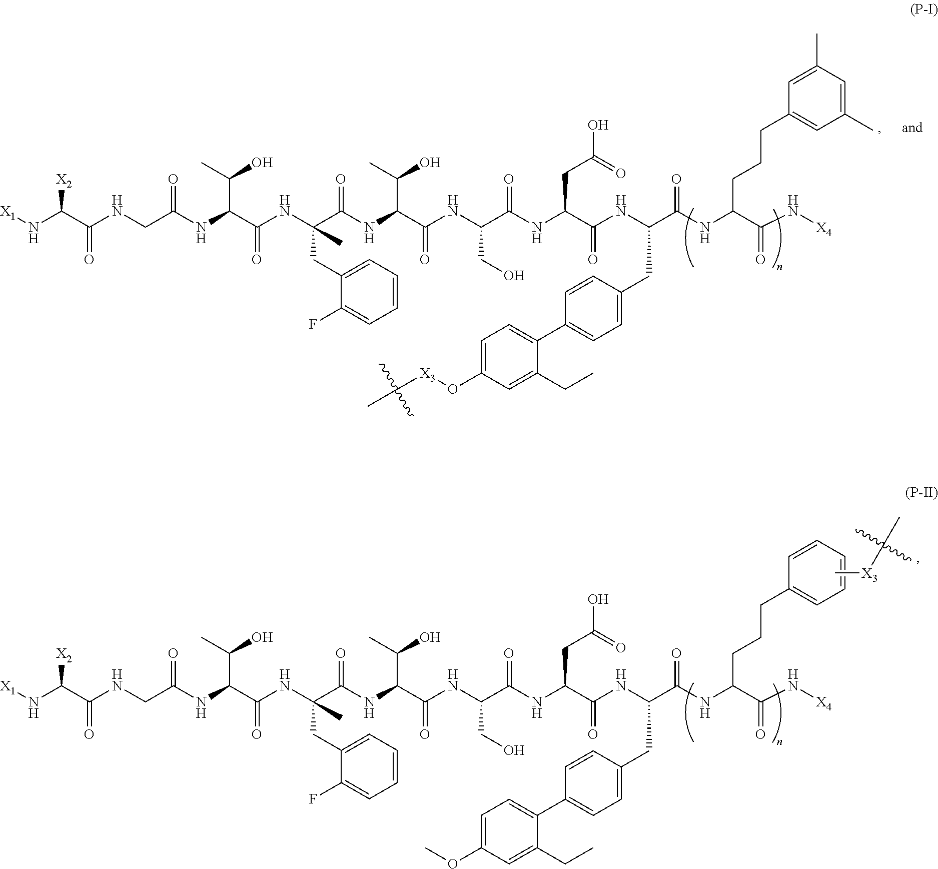

where

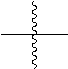

indicates the point of attachment to a linker.

In one embodiment of the invention, the payload has the structure of formula (P-I), shown above, wherein
X₁ is H; X₂ is

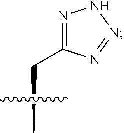

X₃ is selected from —(CH₂)₂₋₆—NH— and —(CH₂)₂₋₆-Tr-, where Tr is a triazole moiety; n is 1, and X₄ is H;
X₁ is

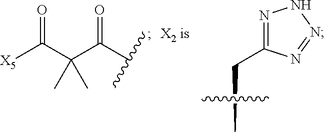

X₃ is —(CH₂)₂₋₆—NH—; n is 1; X₄ is H, and X₅ is selected from —OH, —NH₂, —NH—OH, and

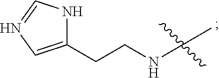 [SMILES: *NCCc1cnc[nH]1]

X₁ is

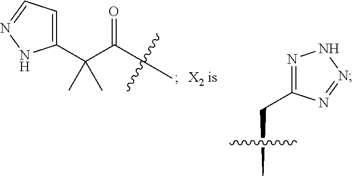

X₃ is —(CH₂)₂₋₆—NH—; n is 1, and X₄ is H;
X₁ is

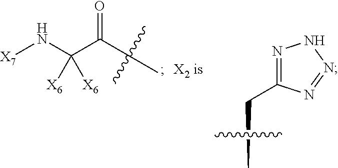

X₃ is —(CH₂)₂₋₆—NH—; n is 1; X₄ is H; X₆ is independently at each occurrence selected from H and —CH₂OH, and X₇ is H;
X₁ is

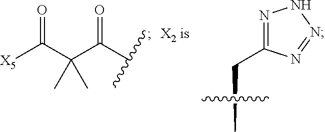

X₃ is —(CH₂)₂₋₆-Tr-, where Tr is a triazole moiety; n is 1; X₄ is H, and X₅ is

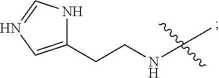 [SMILES: *NCCc1cnc[nH]1]

X₁ is

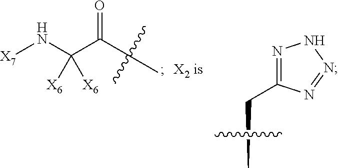

X₃ is —(CH₂)₂₋₆—NH—; n is 1; X₄ is H; X₆ is independently at each occurrence selected from H and —CH₃; X₇ is

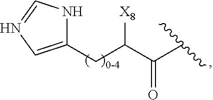

and X₈ is —NH₂;
X₁ is

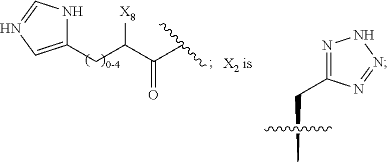

X₃ is —(CH₂)₂₋₆—NH—; n is 1; X₄ is H, and X₃ is H;
X₁ is

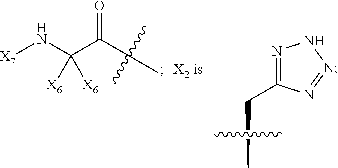

X₃ is —(CH₂)₂₋₆—NH—; n is 1; X₄ is H; X₆ is H at each occurrence; X₇ is

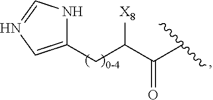

### and X₈ is H;

X₁ is

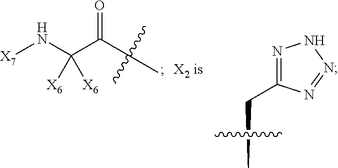

X₃ is —(CH₂)₂₋₆—NH—; n is 1; X₄ is H; X₆ is independently at each occurrence selected from H and —CH₃; X₇ is

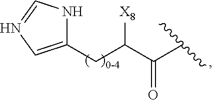

### and X₈ is

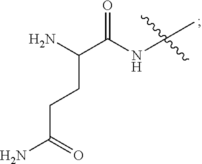 [SMILES: *NC(=O)C(N)CCC(N)=O] [0540] X₁ is

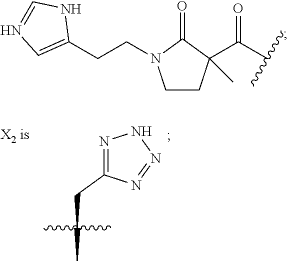

X₃ is —(CH₂)₂₋₆—NH—; n is 1, and X₄ is H;
X₁ is

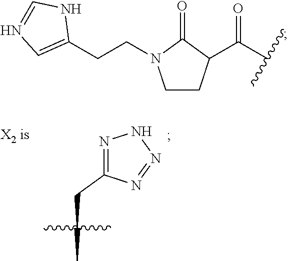

X₃ is —(CH₂)₂₋₆—NH—; n is 1, and X₄ is H;
X₁ is

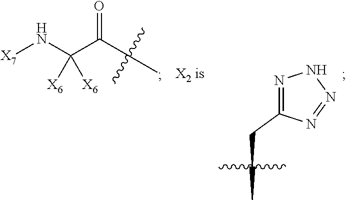

X₃ is —(CH₂)₂₋₆—NH—; n is 1; X₄ is H; X₆ is independently at each occurrence selected from H and —CH₃, and X₇ is

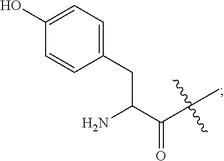 [SMILES: *C(=O)C(N)Cc1ccc(O)cc1]

X₁ is H; X₂ is

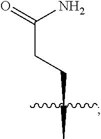

X₃ is —(CH₂)₂₋₆—NH—; n is 1, and X₄ is H;
X₁ is

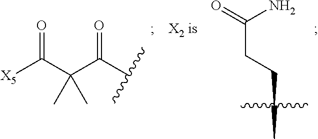

X₃ is —(CH₂)₂₋₆—NH—; n is 1; X₄ is H, and X₅ is

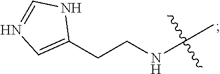 [SMILES: *NCCc1cnc[nH]1]

X₁ is

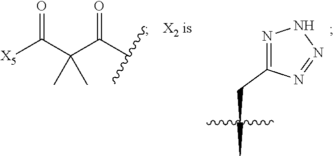

X₃ is —(CH₂)₂₋₆—NH—; n is 0; X₄ is phenyl, and X₅ is

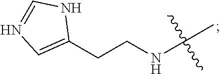 [SMILES: *NCCc1cnc[nH]1]

and
X₁ is

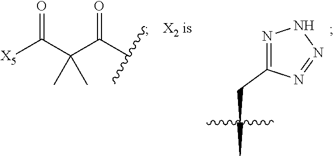

X₃ is —(CH₂)₂₋₆—NH—; n is 1; X₄ is phenyl, and X₅ is

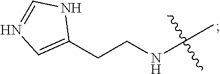 [SMILES: *NCCc1cnc[nH]1]

In one embodiment, the payload has the structure of formula (P-II), shown above, wherein X₁ is

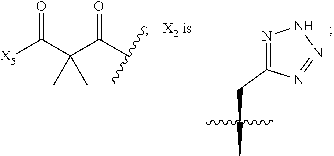

X₃ is —(CH₂)₂₋₆—NH—; X₄ is H, and X₅ is

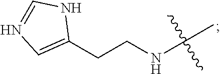 [SMILES: *NCCc1cnc[nH]1]

In one embodiment of the invention, the payloads P according to the present disclosure have a structure selected from (SEQ ID NOS 465, 576, 466-495, 610, 496-497, 611, 498-505, respectively, in order of appearance):

| P and | |
|---|---|
| M # | Structure |
| P1 M2383 | 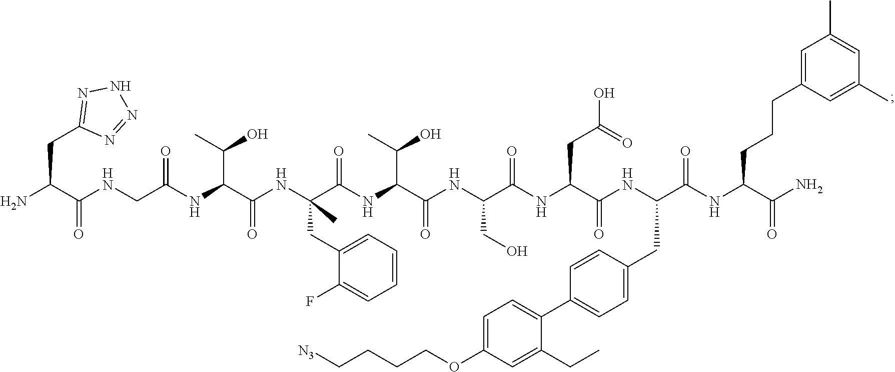 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(N)=O)cc1] |
| P2 M2742 | 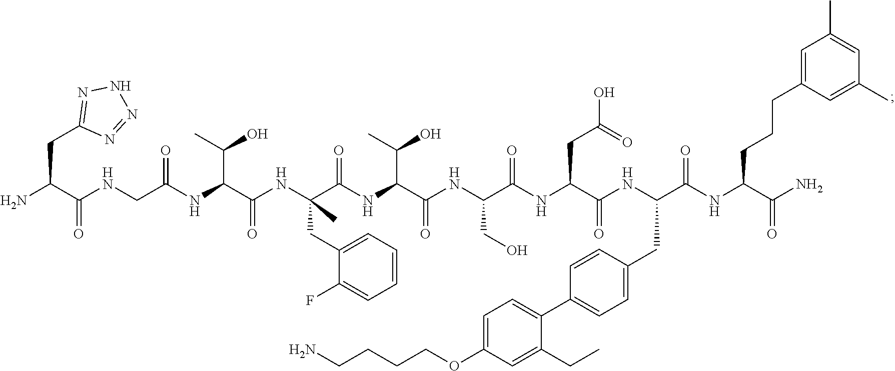 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(N)=O)cc1] |
| P3 M2745 | 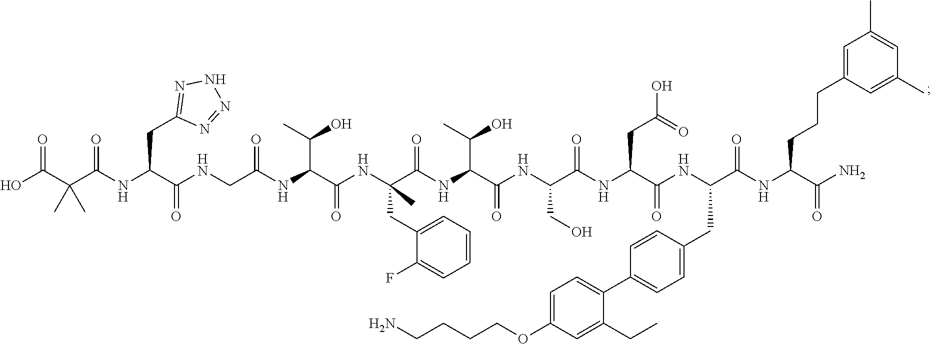 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)O)[C@@H](C)O)[C@@H](C)O)C(=O)N[C@@H](CCCc2cc(C)cc(C)c2)C(N)=O)cc1] |
| P4 M2799 | 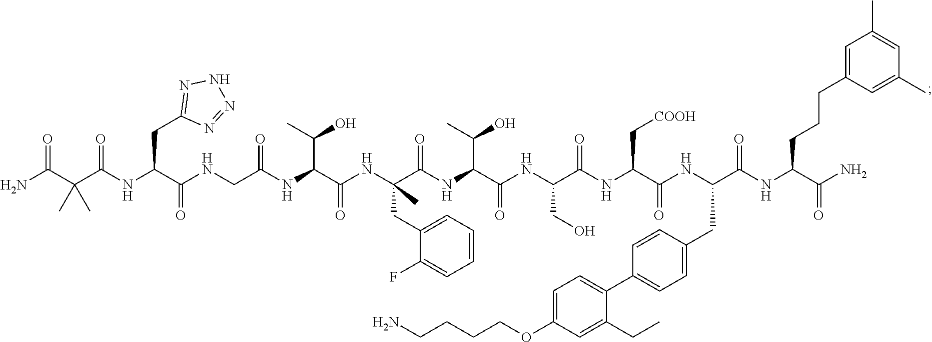 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(N)=O)cc1] |
| P5 M2746 | 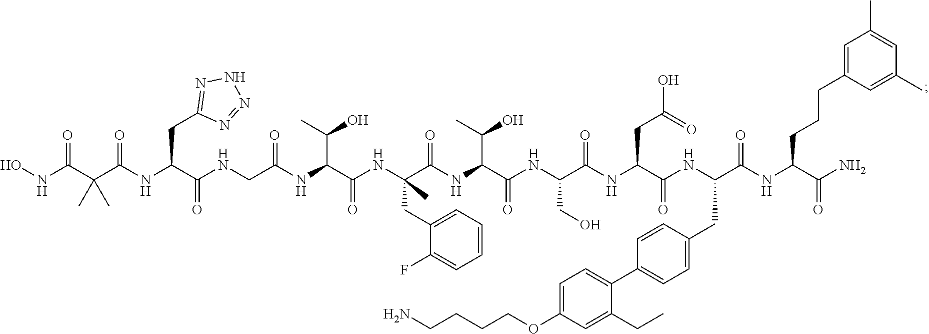 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NO)[C@@H](C)O)[C@@H](C)O)C(=O)N[C@@H](CCCc2cc(C)cc(C)c2)C(N)=O)cc1] |
| P6 M2758 | 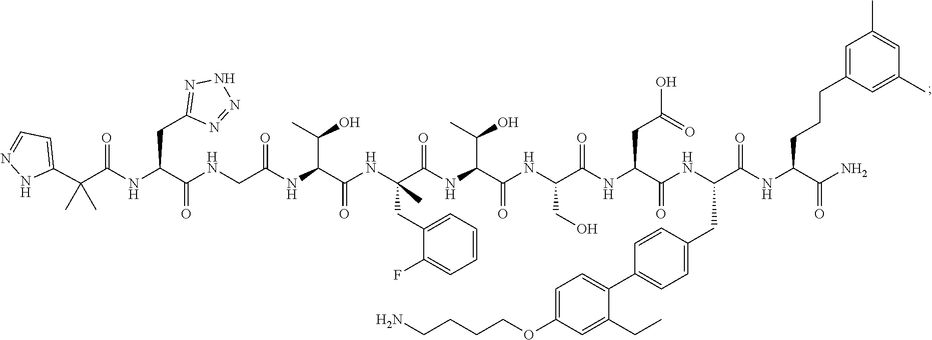 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)c2ccn[nH]2)[C@@H](C)O)[C@@H](C)O)C(=O)N[C@@H](CCCc2cc(C)cc(C)c2)C(N)=O)cc1] |
| P7 M3236 | 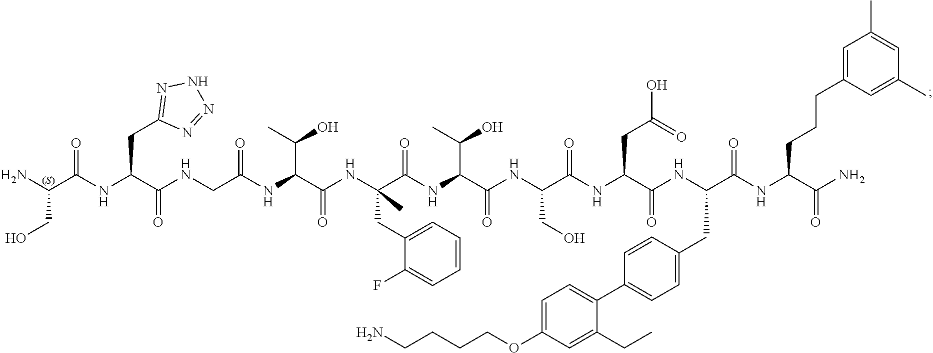 |
| P8 M2361 | 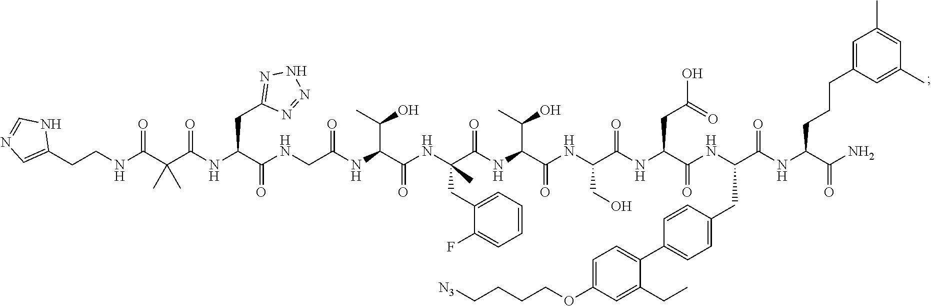 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| P9 M2642 | 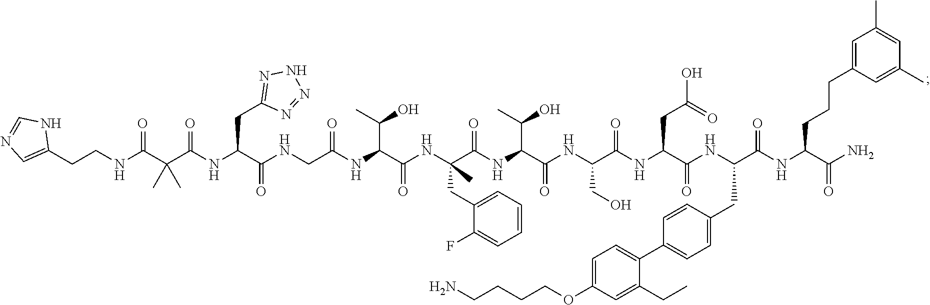 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| P10 M2743 | 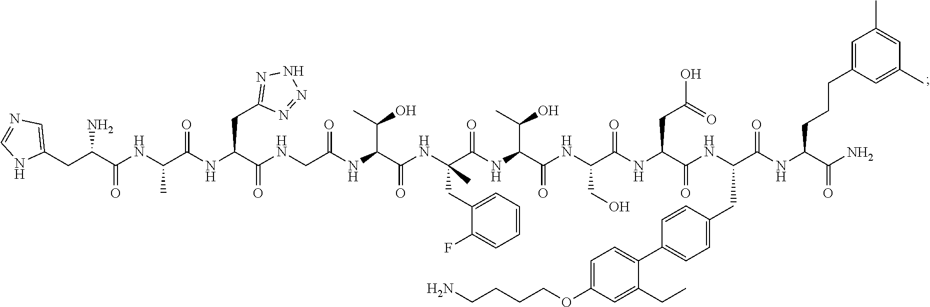 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)[C@H](C)NC(=O)[C@@H](N)Cc2cnc[nH]2)[C@@H](C)O)[C@@H](C)O)C(=O)N[C@@H](CCCc2cc(C)cc(C)c2)C(N)=O)cc1] |
| P11 M2744 | 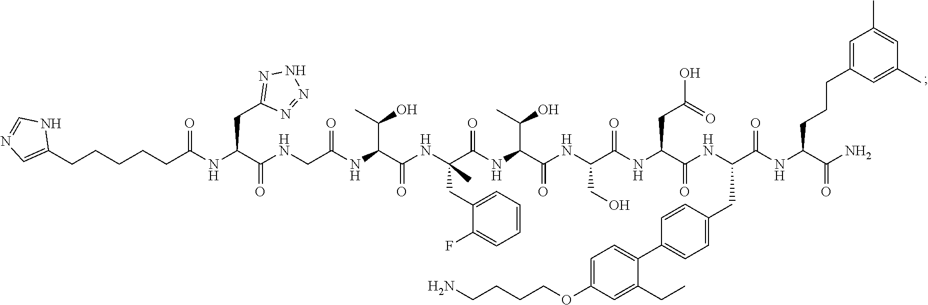 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(N)=O)cc1] |
| P12 M2761 | 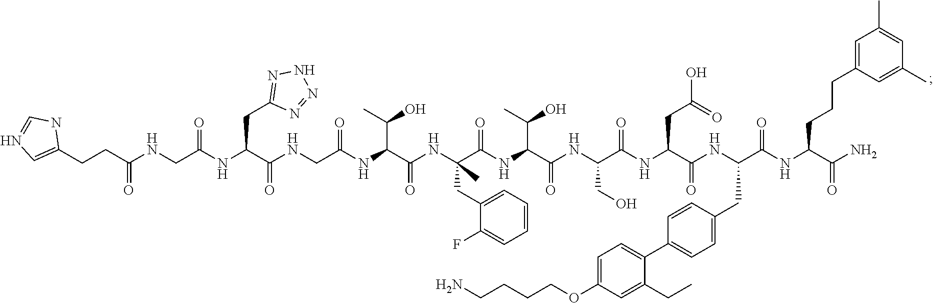 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CNC(=O)CCc2cnc[nH]2)[C@@H](C)O)[C@@H](C)O)C(=O)N[C@@H](CCCc2cc(C)cc(C)c2)C(N)=O)cc1] |
| P13 M2760 | 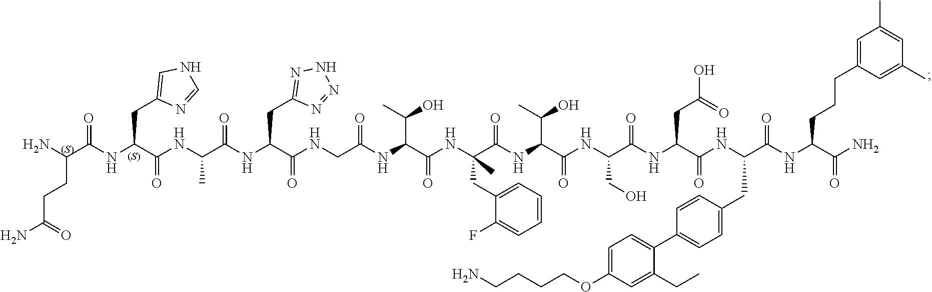 |
| P14 M2797 | 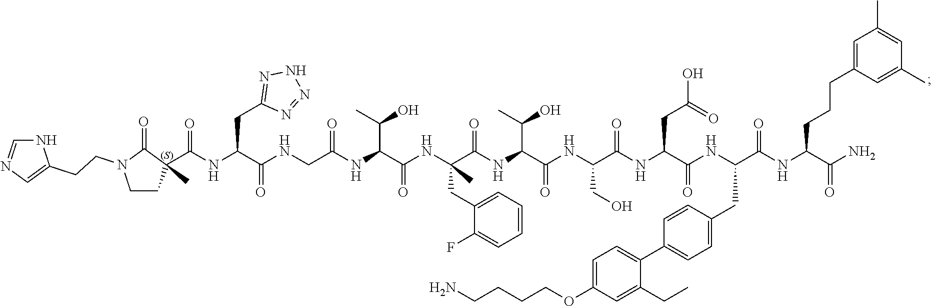 |
| P15 M2798 | 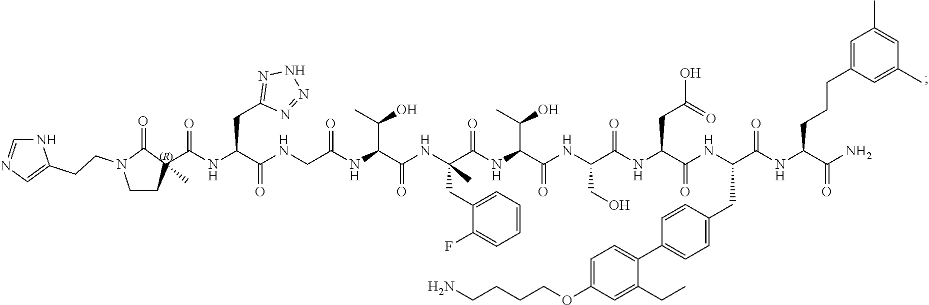 |
| P16 M2985 | 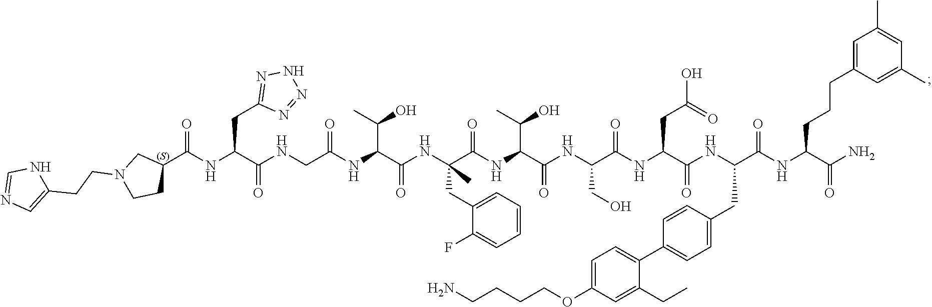 |
| P17 M3240 | 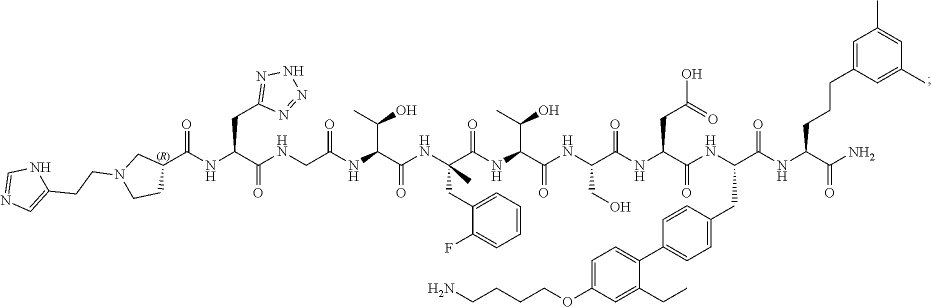 |
| P18 M3056 | 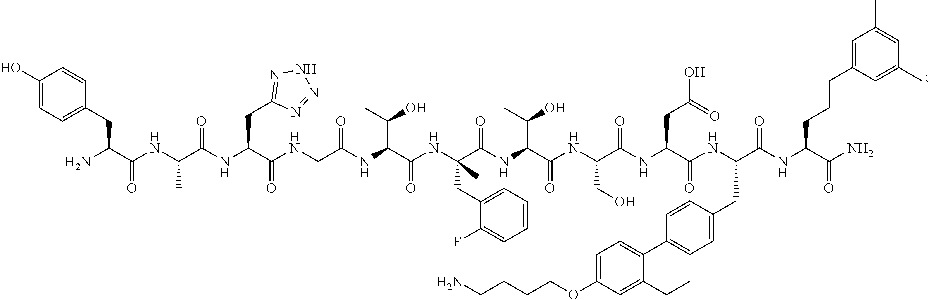 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)[C@H](C)NC(=O)[C@@H](N)Cc2ccc(O)cc2)[C@@H](C)O)[C@@H](C)O)C(=O)N[C@@H](CCCc2cc(C)cc(C)c2)C(N)=O)cc1] |
| P19 M3057 | 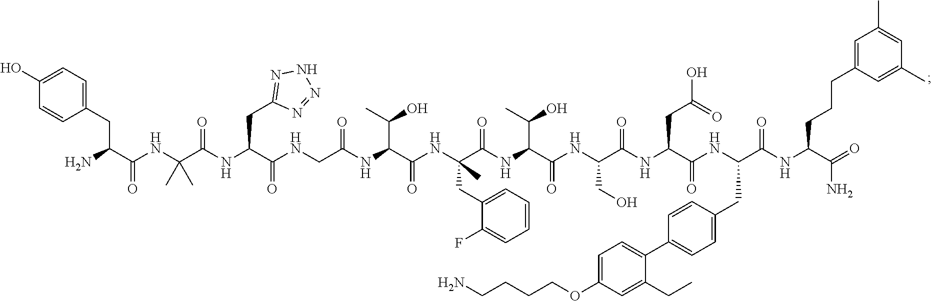 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)[C@@H](N)Cc2ccc(O)cc2)[C@@H](C)O)[C@@H](C)O)C(=O)N[C@@H](CCCc2cc(C)cc(C)c2)C(N)=O)cc1] |
| P20 M2913 | 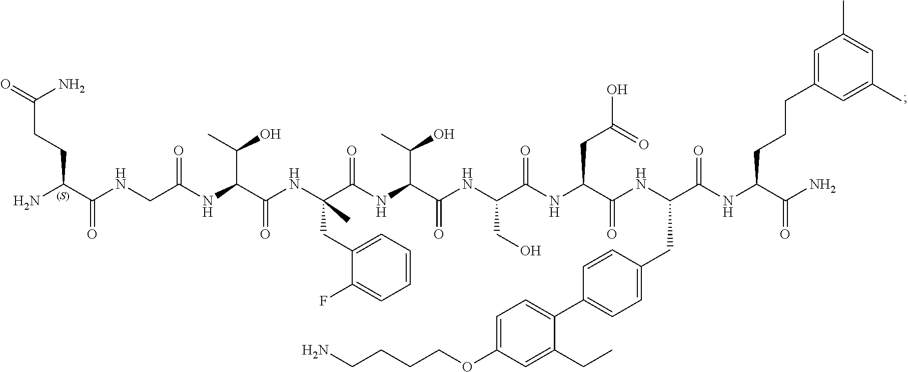 |
| P21 M2912 | 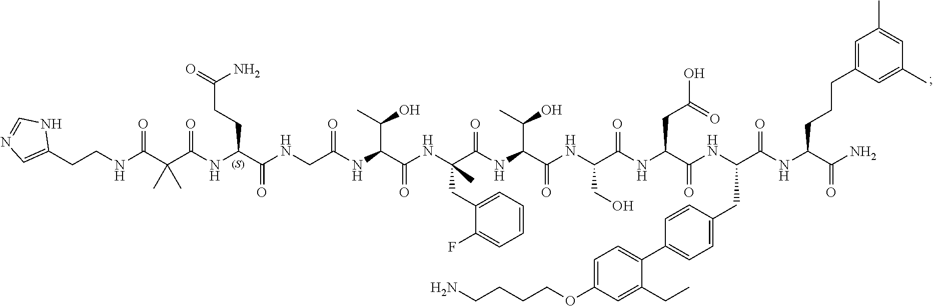 |
| P22 M2801 | 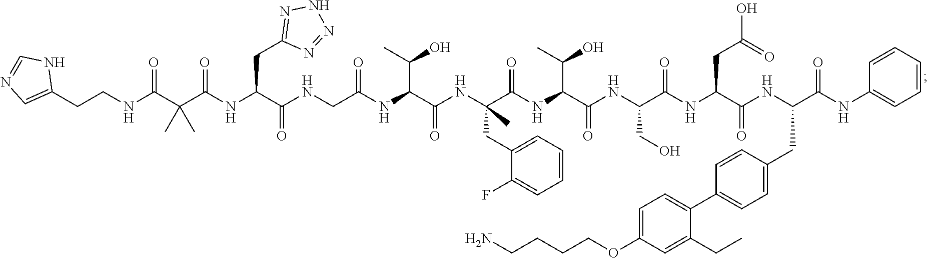 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)Nc2ccccc2)cc1] |
| P23 M2800 | 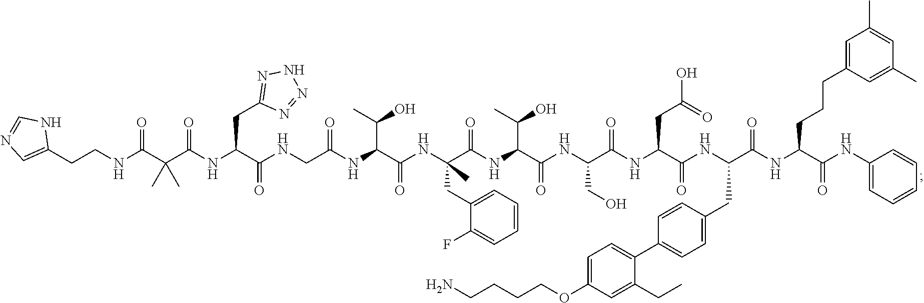 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)Nc2ccccc2)cc1] |
| P24 M3241 | 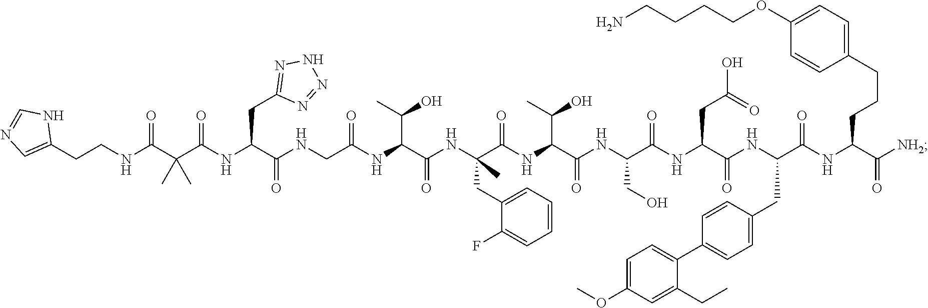 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCN)cc2)C(N)=O)cc1] |
| P25 | 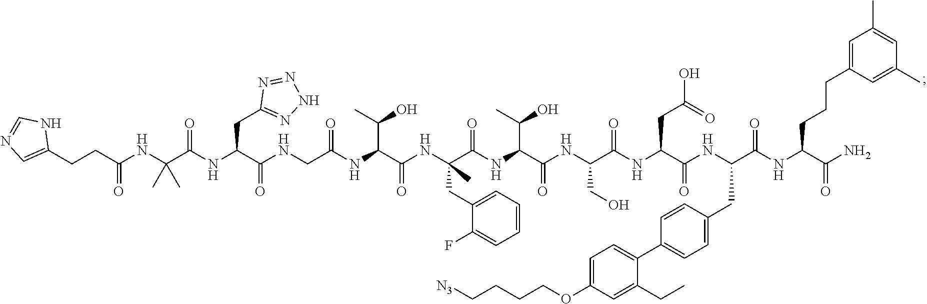 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCc2cnc[nH]2)[C@@H](C)O)[C@@H](C)O)C(=O)N[C@@H](CCCc2cc(C)cc(C)c2)C(N)=O)cc1] |
| P26 | 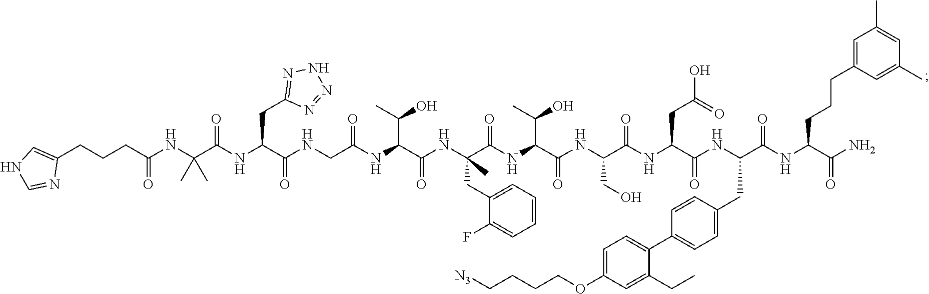 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCc2c[nH]cn2)[C@@H](C)O)[C@@H](C)O)C(=O)N[C@@H](CCCc2cc(C)cc(C)c2)C(N)=O)cc1] |
| P27 | 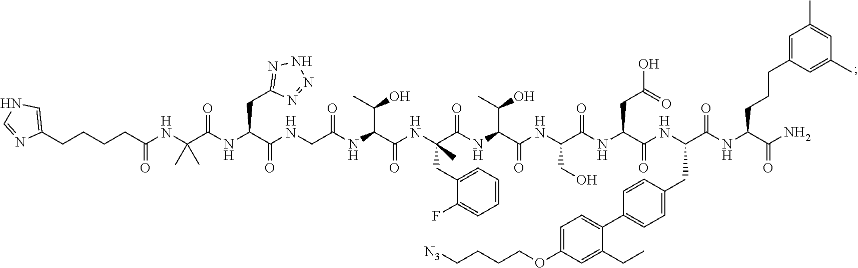 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCCc2c[nH]cn2)[C@@H](C)O)[C@@H](C)O)C(=O)N[C@@H](CCCc2cc(C)cc(C)c2)C(N)=O)cc1] |
| P28 | 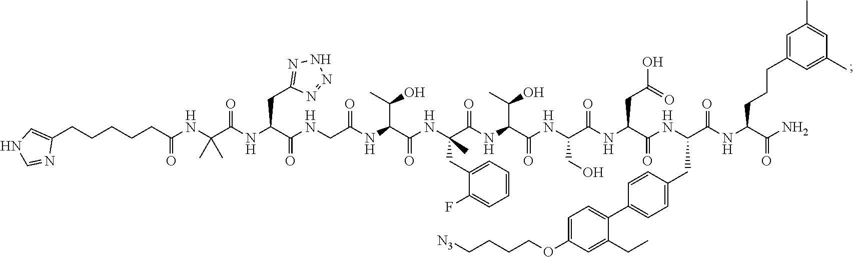 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCCCc2c[nH]cn2)[C@@H](C)O)[C@@H](C)O)C(=O)N[C@@H](CCCc2cc(C)cc(C)c2)C(N)=O)cc1] |
| P29 | 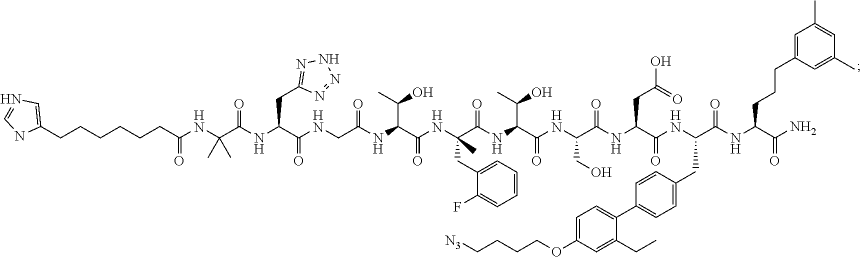 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCCCCc2cnc[nH]2)[C@@H](C)O)[C@@H](C)O)C(=O)N[C@@H](CCCc2cc(C)cc(C)c2)C(N)=O)cc1] |
| P30 | 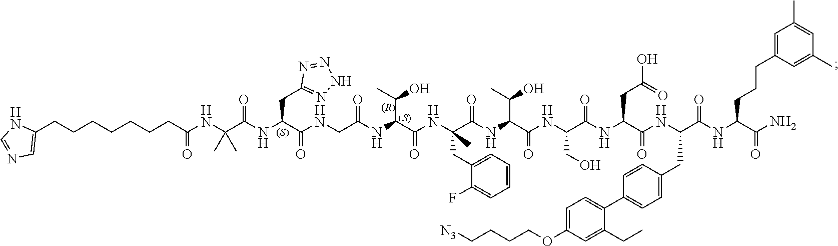 |
| P31 | 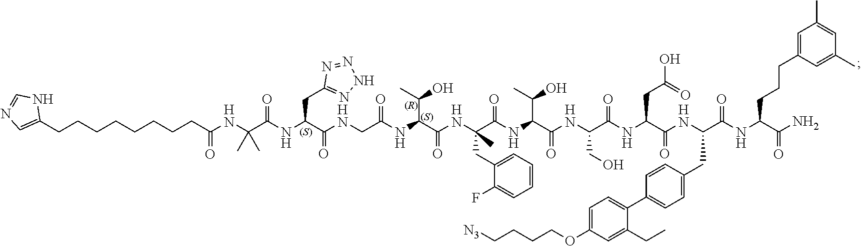 |
| P32 | 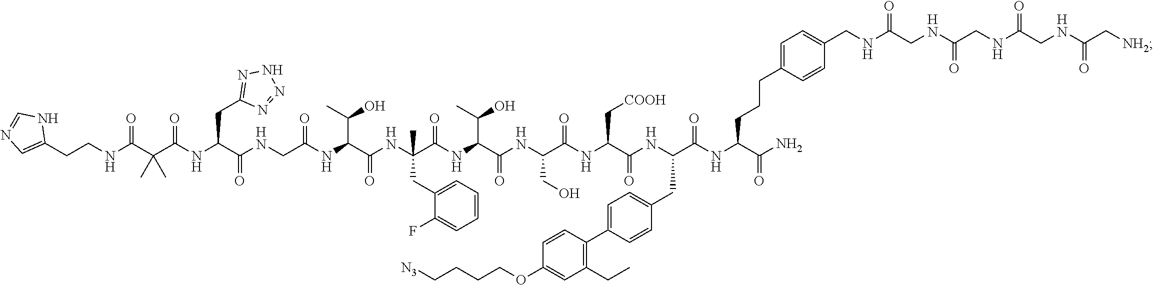 |
| P33 | 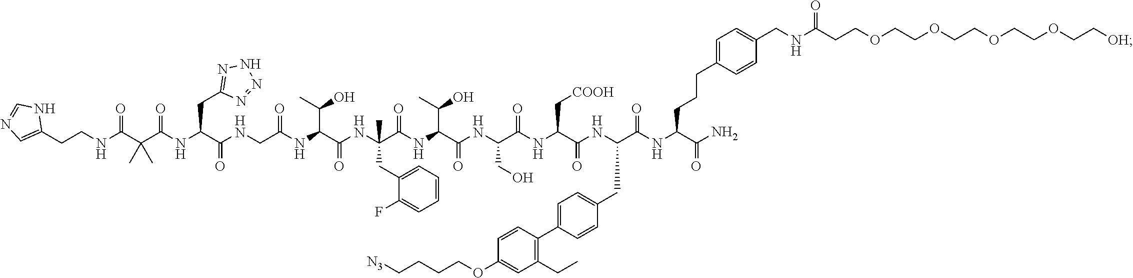 |
| P34 | 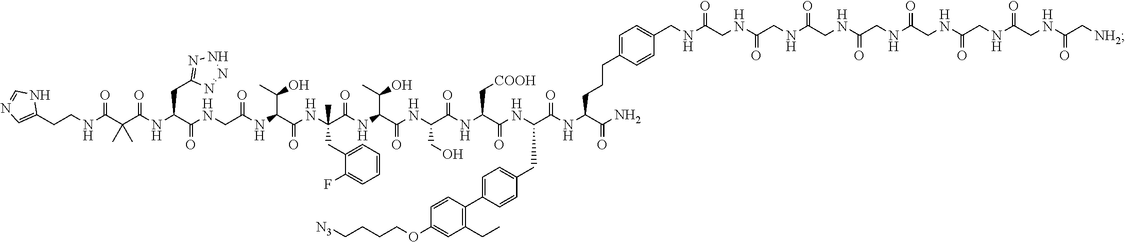 |
| P35 | 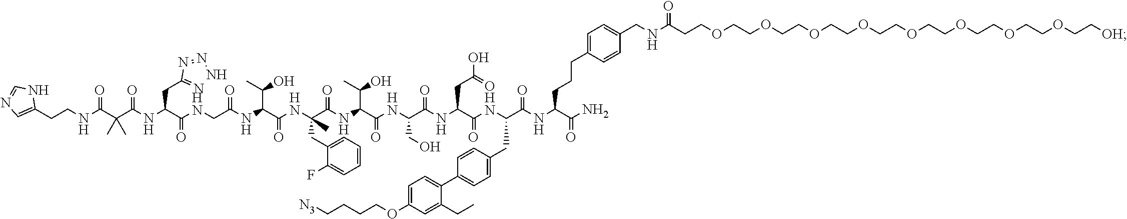 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(CNC(=O)CCOCCOCCOCCOCCOCCOCCOCCOCCO)cc2)C(N)=O)cc1] |
| P36 | 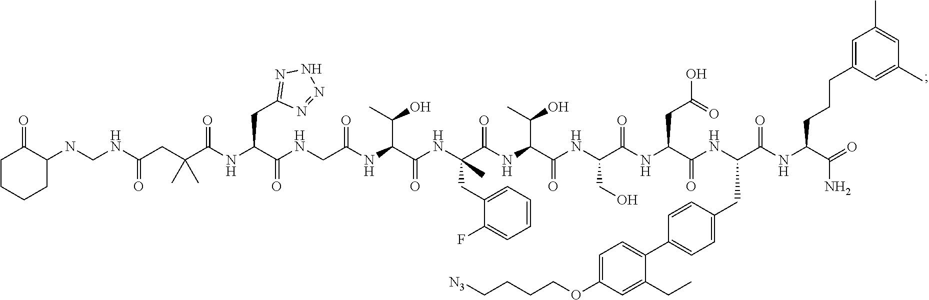 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)CC(=O)NCNC2CCCCC2=O)[C@@H](C)O)[C@@H](C)O)C(=O)N[C@@H](CCCc2cc(C)cc(C)c2)C(N)=O)cc1] |
| P37 | 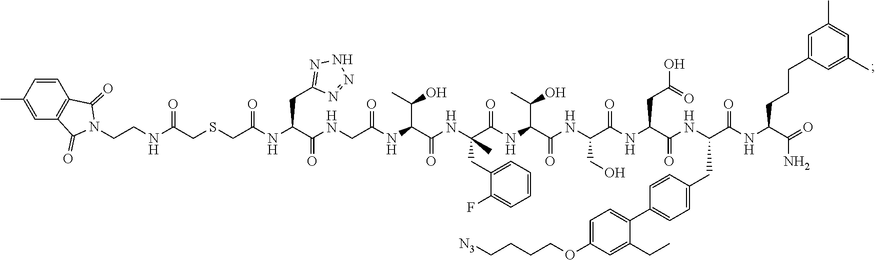 |
| P38 | 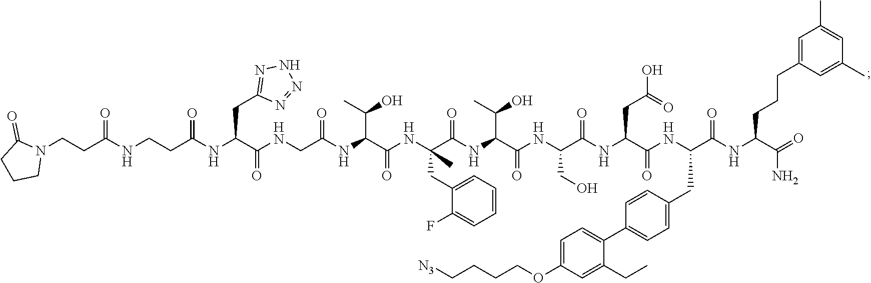 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCNC(=O)CCN2CCCC2=O)[C@@H](C)O)[C@@H](C)O)C(=O)N[C@@H](CCCc2cc(C)cc(C)c2)C(N)=O)cc1] |
| P39 | 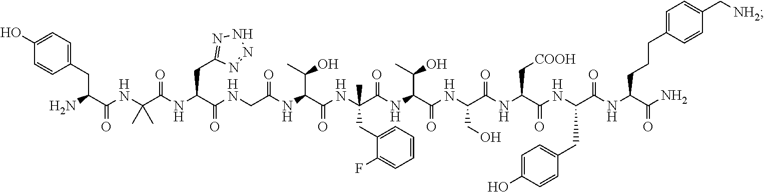 |
| P40 | 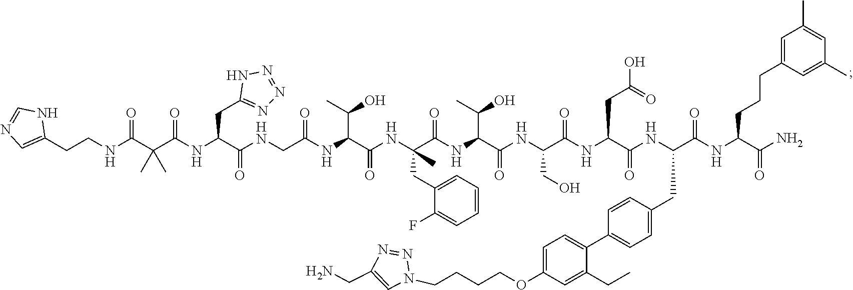 [SMILES: CCc1cc(OCCCCn2cc(CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| P41 | 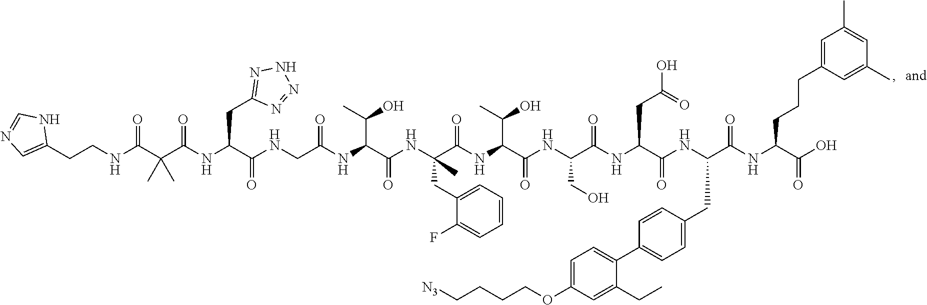 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| P42 | 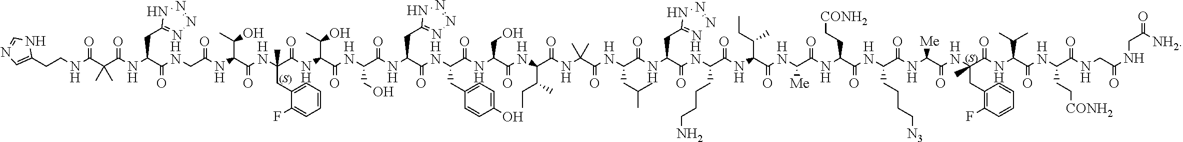 |

In one embodiment of the invention, the payloads as described above have the following properties:

| | | | | RT on | | Plasma |
|---|---|---|---|---|---|---|
| | Molecular | | M/Z 100% | HPLC | | stability |
| P# | Formula | MW | (M + H) | (15 min) | CLogP | t½ (hr) |
| P1 | C₆₅H₈₆FN₁₇O₁₅ | 1364.48 | 860.2 [M + 2H]²⁺ | 10.15 | 4.94 ± 0.99 | |
| | | | 1365.6 [M + H]⁺ | | | |
| P2 | C₆₅H₈₈FN₁₅O₁₅•2CF₃COOH | 1566.53 | 670.0 [M + 2H]²⁺ | 7.77 | 4.08 ± 0.98 | 10.7 |
| P3 | C₇₀H₉₄FN₁₅O₁₈•CF₃COOH | 1566.61 | 1454.1 [M + H]⁺ | 8.10 | 4.54 ± 1.00 | >57.8 |
| | | | 727.3 [M + 2H]²⁺ | | | |
| P4 | C₇₀H₉₅FN₁₆O₁₇•CF₃COOH | 1565.62 | 1452.4 [M + H]⁺ | 7.90 | 3.59 ± 1.00 | >57.8 |
| | | | 726.7 [M + 2H]²⁺ | | | |
| P5 | C₇₀H₉₅FN₁₆O₁₈ | 1467.6 | 1468.0 [M + H]⁺ | 8.00 | 3.23 ± 1.00 | |
| | | | 735.2 [M + 2H]²⁺ | | | |
| P6 | C₇₂H₉₆FN₁₇O₁₆•2CF₃COOH | 1702.68 | 738.2 [M + 2H]²⁺ | 8.16 | 4.38 ± 1.00 | >57.8 |
| P7 | C₆₈H₉₃FN₁₆O₁₇•2CF₃COOH | 1653.61 | 713.8 [M + 2H]²⁺ | 7.58 | 3.16 ± 1.00 | |
| P8 | C₇₅H₉₉FN₂₀O₁₇ | 1571.71 | 786.38 [M + 2H]²⁺ | 10.14 | 4.43 ± 1.02 | |
| | | | 1571.76 [M + H]⁺ | | | |
| P9 | C₇₅H₁₀₁FN₁₈O₁₇•2CF₃COOH | 1773.76 | 773.9 [M + 2H]²⁺ | 7.44 | 3.57 ± 1.01 | >57.8 |
| P10 | C₇₄H₁₀₀FN₁₉O₁₇•3CF₃COOH | 1888.77 | 516.4 [M + 3H]³⁺ | 7.46 | 2.95 ± 1.02 | 1.8 |
| | | | 774.2 [M + 2H]²⁺ | | | |
| P11 | C₇₄H₁₀₀FN₁₇O₁₆•2CF₃COOH | 1730.74 | 752.3 [M + 2H]²⁺ | 7.72 | 5.04 ± 0.99 | >57.8 |
| P12 | C₇₃H₉₇FN₁₈O₁₇•2CF₃COOH | 1745.71 | 759.8 [M + 2H]²⁺ | 7.65 | 3.03 ± 1.01 | >57.8 |
| P13 | C₇₉H₁₀₈FN₂₁O₁₉•3CF₃COOH | 2016.9 | 559.1 [M + 3H]³⁺ | 7.37 | 1.81 ± 1.04 | 16.9 |
| | | | 838.3 [M + 2H]²⁺ | | | |
| P14 | C₇₆H₁₀₁FN₁₈O₁₇ | 1557.72 | 779.9 [M + 2H]²⁺ | 7.60 | 3.68 ± 1.01 | |
| P15 | C₇₆H₁₀₁FN₁₈O₁₇•2(CF₃COOH) | 1785.77 | 779.8 [M + 2H]²⁺ | 7.62 | 3.68 ± 1.01 | |
| P16 | C₇₅H₁₀₁FN₁₈O₁₆•2(CF₃COOH) | 1757.76 | 765.7[M + 2H]²⁺ | 7.18 | 3.68 ± 1.01 | |
| P17 | C₇₅H₁₀₁FN₁₈O₁₆•2(CF₃COOH) | 1757.76 | 766.3 [M + 2H]²⁺ | 7.36 | 3.68 ± 1.01 | |
| P18 | C₇₇H₁₀₂FN₁₇O₁₈ | 1572.74 | 787.3 [M + 2H]²⁺ | 7.68 | 4.60 ± 1.01 | |
| P19 | C₇₈H₁₀₄FN₁₇O₁₈ | 1586.76 | 794.4 [M + 2H]²⁺ | 7.81 | 4.95 ± 1.01 | >57.8 |
| P20 | C₆₆H₉₁FN₁₂O₁₆ | 1327.5 | 1327.7 [M + H]⁺ | 7.40 | 3.41 ± 1.00 | |
| P21 | C₇₆H₁₀₄FN₁₅O₁₈•2CF₃COOH | 1762.77 | 768.3 [M + 2H]²⁺ | 7.44 | 3.92 ± 0.97 | >57.8 |
| P22 | C₆₈H₈₈FN₁₇O₁₆ | 1418.53 | 710.2 [M + 2H]²⁺ | 7.06 | 3.03 ± 0.99 | |
| P23 | C₈₁H₁₀₅FN₁₈O₁₇ | 1621.81 | 811.9 [M + 2H]²⁺ | 8.30 | 6.00 ± 1.01 | |
| P24 | C₇₄H₉₉FN₁₈O₁₈•2CF₃COOH | 1775.73 | 774.8 [M + 2H]²⁺ | 11.00 | 2.56 ± 1.01 | |
| | | | | (20 min) | | |
| P25 | C₇₅H₉₉FN₂₀O₁₇ | 1570.8 | 786.7 [M + 2H]²⁺ | 9.54 | 4.58 ± 1.01 | |
| P26 | C₇₆H₁₀₁FN₂₀O₁₇ | 1584.8 | 793.7 [M + 2H]²⁺ | 9.55 | 5.09 ± 1.01 | |
| P27 | C₇₇H₁₀₃FN₂₀O₁₇ | 1598.8 | 800.39 [M + 2H]²⁺ | 9.58 | 5.54 ± 1.01 | |
| P28 | C₇₈H₁₀₅FN₂₀O₁₇ | 1612.8 | 807.40 [M + 2H]²⁺ | 9.60 | 5.98 ± 1.01 | |
| P29 | C₇₉H₁₀₇FN₂₀O₁₇ | 1626.8 | 814.40 [M + 2H]²⁺ | 9.61 | 6.51 ± 1.01 | |
| P30 | C₈₁H₁₁₁FN₂₀O₁₇ | 1768.8 | 821.4 [M + 2H]²⁺ | 9.63 | 7.57 ± 1.01 | |
| P31 | C₈₁H₁₁₁FN₂₀O₁₇ | 1654.8 | 828.8 [M + 2H]²⁺ | 9.80 | 7.57 ± 1.01 | |
| P32 | C₈₂H₁₁₀FN₂₅O₂₁ | 1799.8 | 900.91 [M + 2H]²⁺ | 7.23 | −0.30 ± 1.06 | |
| P33 | C₈₅H₁₁₈FN₂₁O₂₃ | 1819.9 | 911.40 [M + 2H]²⁺ | 7.96 | 0.25 ± 1.05 | |
| P34 | C₉₀H₁₂₂FN₂₉O₂₅ | 2027.9 | 1015.2 [M + 2H]²⁺ | 7.15 | −2.76 ± 1.10 | |
| P35 | C₉₃H₁₃₄FN₂₁O₂₇ | 1996.0 | 999.4 [M + 2H]²⁺ | 7.88 | −1.18 ± 1.07 | |
| P36 | C₇₈H₁₀₆FN₁₉O₁₈ | 1615.8 | 809.3 [M + 2H]²⁺ | 5.25 | 4.76 ± 1.02 | |
| P37 | C₈₀H₁₀₀FN₁₉O₁₉S | 1681.7 | 842.3 [M + 2H]²⁺ | 4.84 | 7.01 ± 1.03 | |
| P38 | C₇₅H₁₀₀FN₁₉O₁₈ | 1573.8 | 788.20 [M + 2H]²⁺ | 9.93 | 3.49 ± 1.02 | |
| P39 | C₆₅H₈₆FN₁₇O₁₈ | 1411.63 | 1412.7 [M + H]⁺, | 6.89 | −0.24 +/− 1.01 | |
| | | | 707.3 [M + 2H]²⁺ | | | |
| P40 | C₇₈H₁₀₄FN₂₁O₁₇ | 1625.79 | 814.2 [M + 2H]²⁺ | 6.94 | 2.98 +/− 1.40 | |
| P41 | C₇₅H₉₈FN₁₉O₁₈ | 1571.73 | 787.3 [M + 2H]²⁺ | 9.66 | 5.40 +/− 1.02 | |
| P42 | C₁₂₆H₁₉₀F₂N₄₆₀₃₃ | 2913.46 | 1458.2 [M + 2H]²⁺ | 7.17 | −3.08 +/− 1.57 | |

In some embodiments of the invention, the payloads of the present disclosure are amenable to conjugation with a binding agent (e.g., antibody or antigen-binding fragment thereof e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280).

In one embodiment of the invention, the present disclosure provides reactive linker-payloads (L-P) comprising payloads P as described above and linkers capable of covalently attaching to an antibody or an antigen-binding fragment thereof (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280).

In one embodiment of the invention, the linker-payload according to the present disclosure has a structure of Formula (C) (SEQ ID NO: 512):

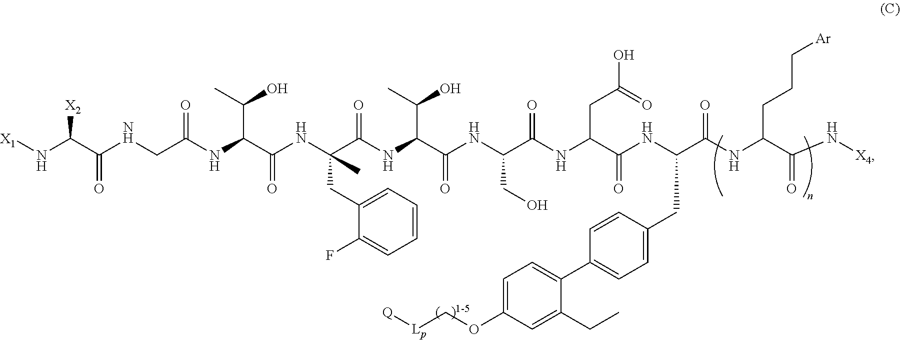

wherein:
Lp is absent or a linker comprising one or more of

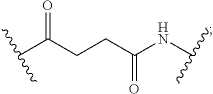 [SMILES: *NC(=O)CCC(*)=O]

a carbamate group; a cyclodextrin; a polyethylene glycol (PEG) segment having 1 to 36 —CH₂CH₂O— (EG) units; a —(CH₂)₂₋₂₄— chain; a triazole; one or more amino acids selected from glycine, serine, glutamic acid, alanine, valine, and proline, and combinations thereof;
Q is a moiety selected from —NH₂, —N₃

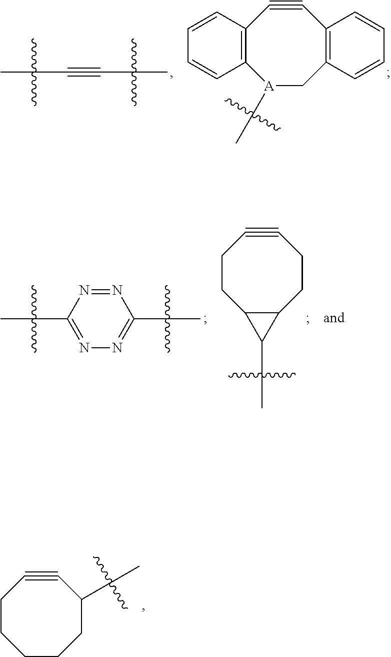

where A is C or N;
X₁ is selected from H;

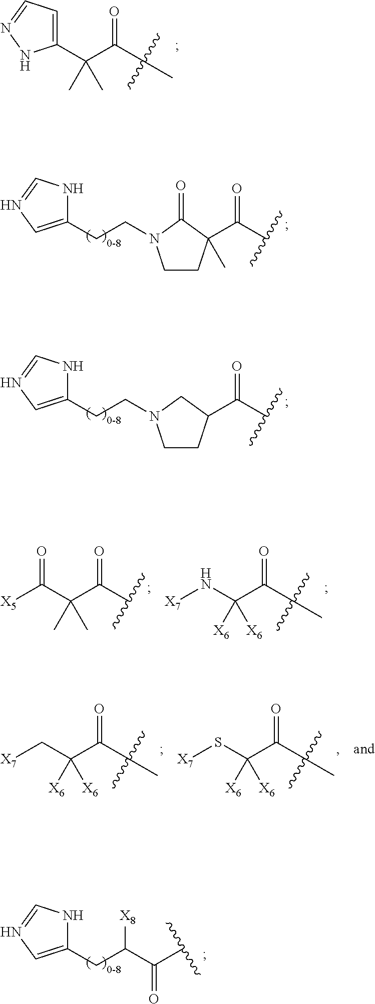

X₂ is selected from

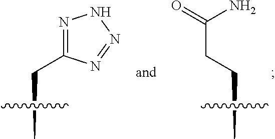

X₃ is selected from —CH₃, —(CH₂)₂₋₆—NH₂, —(CH₂)₂₋₆—N₃, and —(CH₂)₂₋₆-Tr-(CH₂)₁₋₆—NH₂, where Tr is a triazole moiety;
n is 0 or 1;
X₄ is selected from —NH₂, —OH and —N(H)(phenyl);
X₅ is selected from —OH, —NH₂, —NH—OH, and

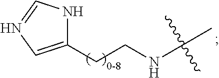

X₆ is independently at each occurrence selected from H, —OH, —CH₃, and —CH₂OH;
X₇ is selected from H,

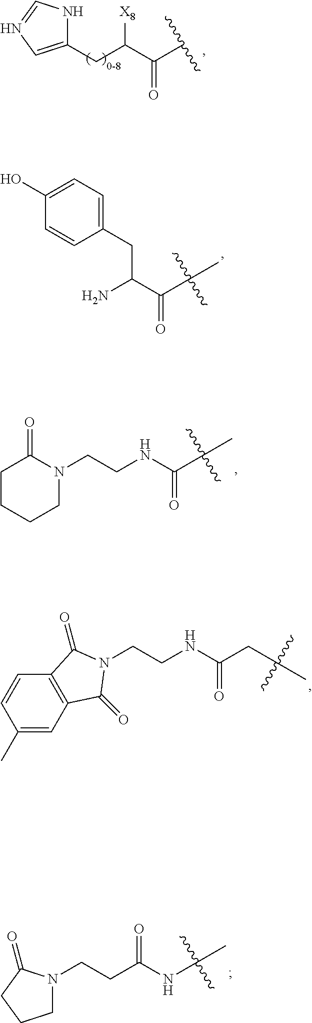

X₈ is selected from H, —OH, —NH₂, and

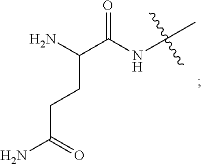 [SMILES: *NC(=O)C(N)CCC(N)=O]

Ar is selected from

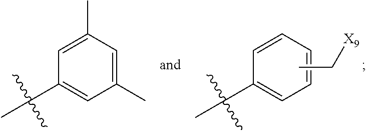

X₉ is selected from —NH₂,

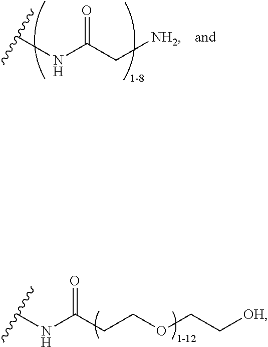

and
m is an integer from 1 to 4
   or a pharmaceutically acceptable salt thereof.

In one embodiment, the linker-payload according to the present disclosure has a structure of Formula (III) (SEQ ID NO: 513):

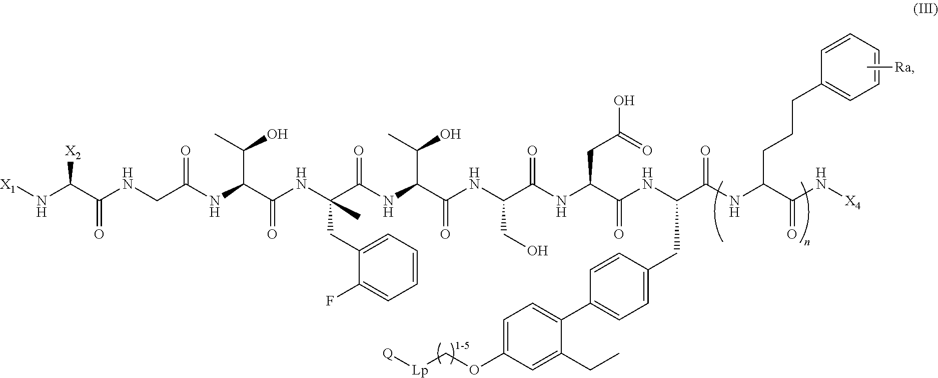

wherein:
Lp is absent or a linker comprising one or more of

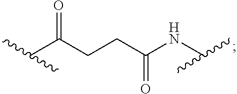 [SMILES: *NC(=O)CCC(*)=O]

a carbamate group; a cyclodextrin; a polyethylene glycol (PEG) segment having 1 to 36 —CH₂CH₂O— (EG) units; one or more amino acids selected from glycine, serine, glutamic acid, alanine, valine, and proline, and combinations thereof;
Q is a moiety selected from —N₃,

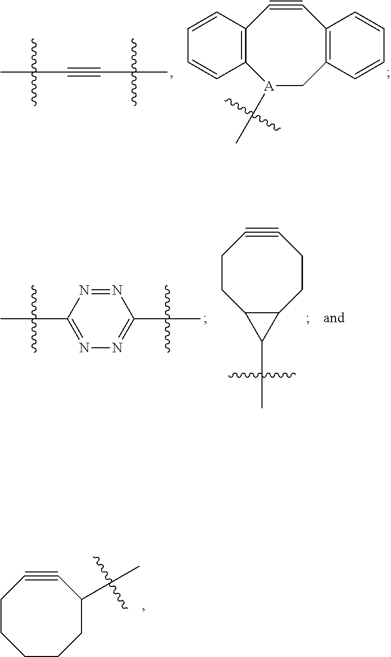

where A is C or N;
X₁ is selected from H;

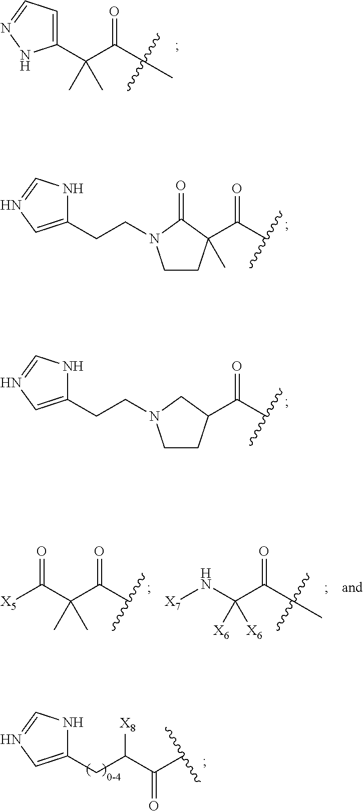

X₂ is selected from

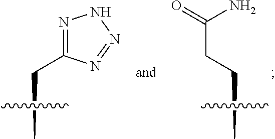

X₃ is selected from —(CH₂)₂₋₆—NH₂, —(CH₂)₂₋₆—N₃, and —CH₃, with the proviso that when X₃ is —CH₃, n is 1 and Ra in at least one occurrence is selected from —(CH₂)₂₋₆—NH₂ and —(CH₂)₂₋₆—N₃;
n is 0 or 1;
Ra is independently at each occurrence selected from H, —CH₃, —(CH₂)₂₋₆—NH₂, and —(CH₂)₂₋₆—N₃;
X₄ is selected from H and phenyl;
X₅ is selected from —OH, —NH₂, —NH—OH, and

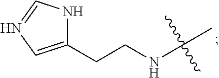 [SMILES: *NCCc1cnc[nH]1]

X₆ is independently at each occurrence selected from H, —OH, —CH₃, and —CH₂OH;
X₇ is selected from H,

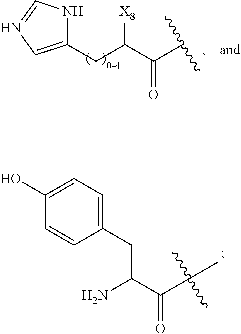

X₈ is selected from H, —OH, —NH₂, and

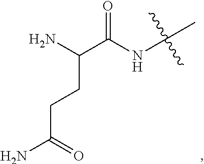 [SMILES: *NC(=O)C(N)CCC(N)=O]

and pharmaceutically acceptable salts thereof.

In one embodiment of the invention, the linker-payload LP comprises a cyclodextrin moiety. In some embodiments of the invention, the linker-payload LP comprising a cyclodextrin moiety exhibits GLP1R agonism activity.

In one embodiment of the invention, the linker-payloads LP according to the present disclosure have the structure selected from the group consisting of (SEQ ID NOS 514, 514, 514, 514, 514-516, 515-519, 519, 519-530, 529-532, 515-516, 534-536, 538, 536-537, 521, 539-541, 541-543, 519, 544, 544-566 and 569, respectively, in order of appearance), respectively, in order of appearance):

| LP and | | |
|---|---|---|
| M# | Name | Structure |
| LP1 M2546 | DIBAC- suc-PEG4- P9 | 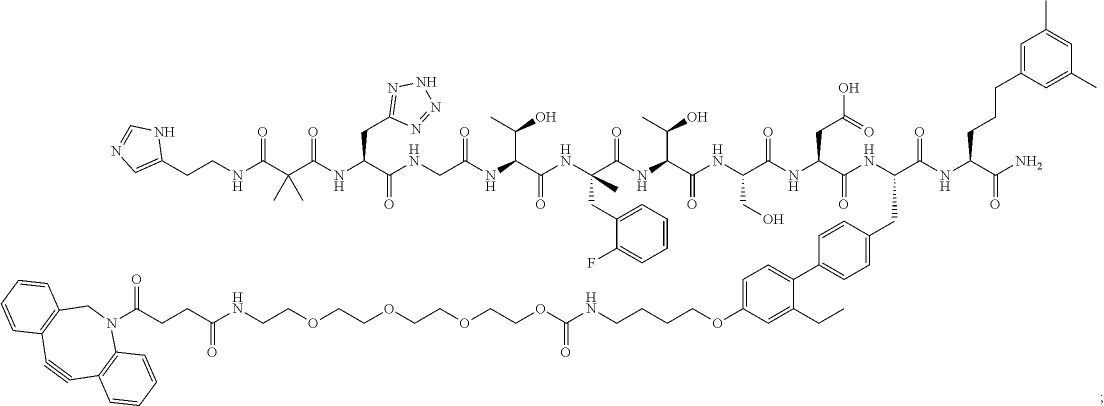 [SMILES: CCc1cc(OCCCCNC(=O)OCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP2 M2663 | DIBAC- suc- PEG8- P9 | 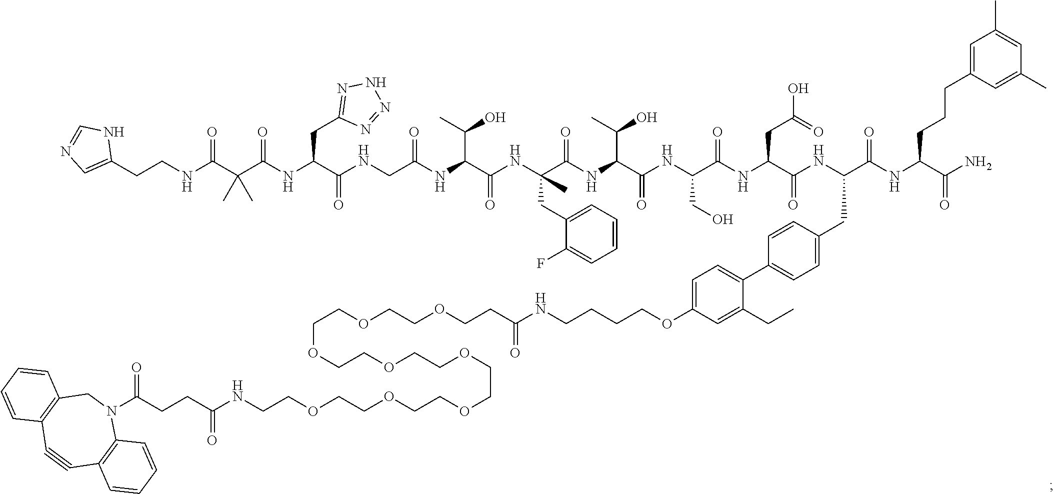 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP3 M2494 | DIBAC- suc- PEG12-P9 | 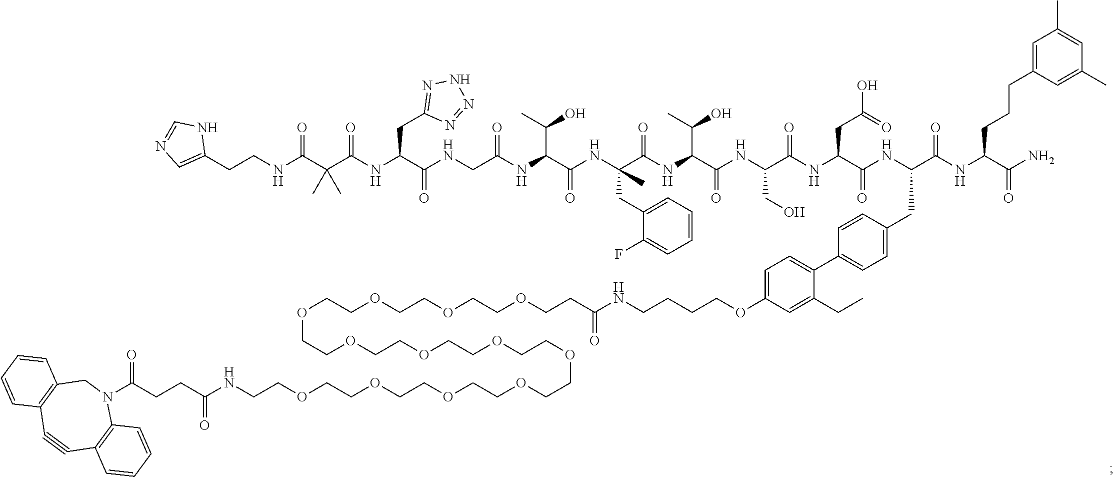 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP4 | DIBAC- | [symbol] ; |
| M2399 | suc- | |
| | PEG24- P9 | |
| LP5 | BCN- | [symbol] ; |
| M3152 | PEG4- | |
| | carba- | |
| | mate | |
| | P9 | |
| LP6 | DIBAC- | [symbol] ; |
| M2747 | suc-G4S- | |
| | P9 | |
| LP6A M2739 | G4S-P9 | 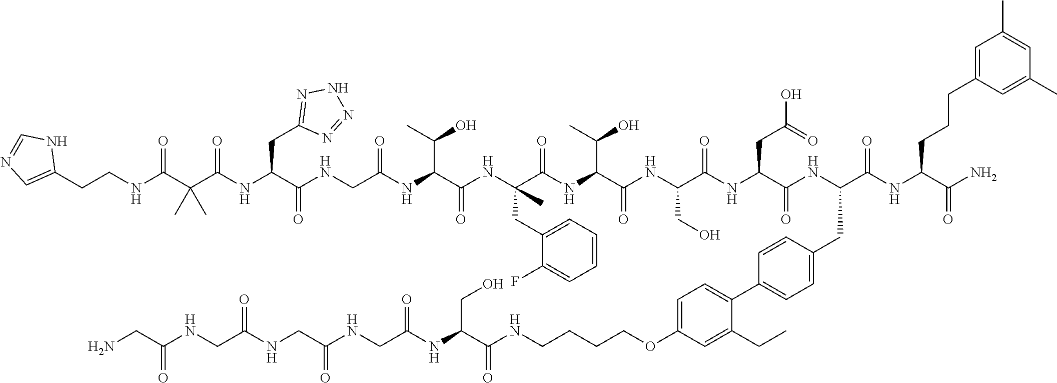 [SMILES: CCc1cc(OCCCCNC(=O)[C@H](CO)NC(=O)CNC(=O)CNC(=O)CNC(=O)CN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP7 M3053 | DIBAC- suc-SG4- P9 | 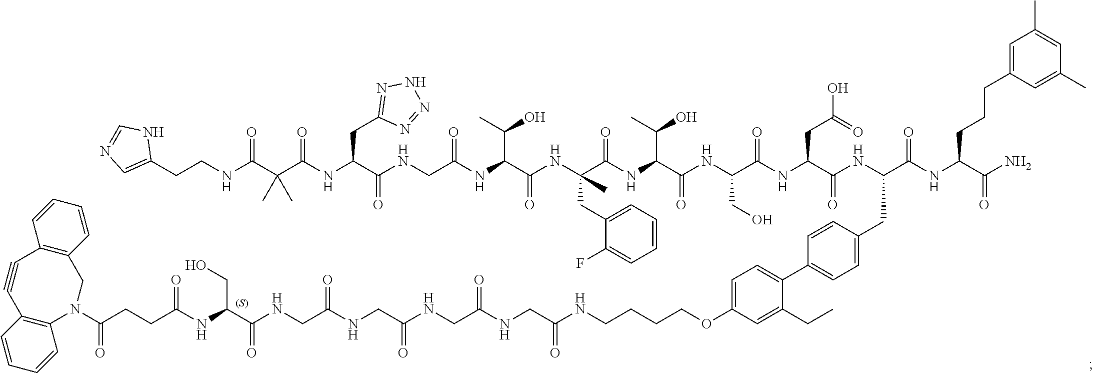 |
| LP8 | DIBAC- suc-PEG4- triazole-P8 | 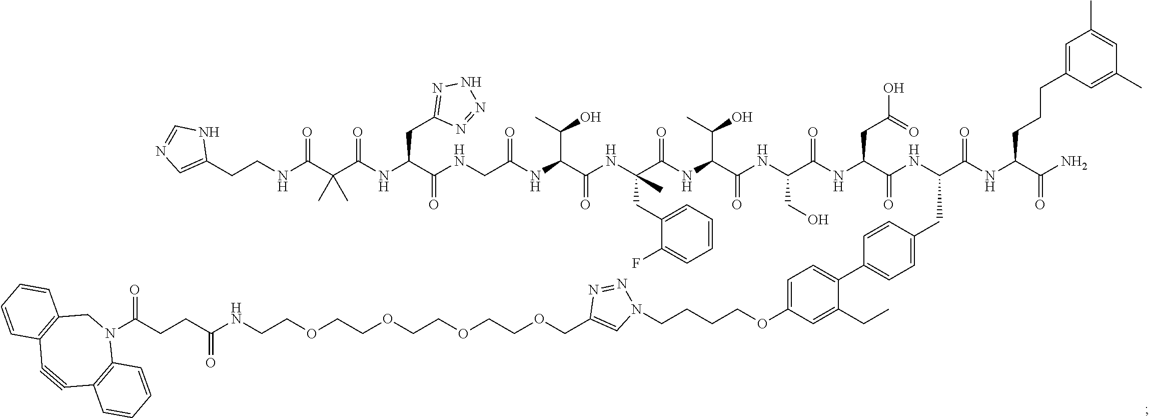 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP9 M3151 | BCN- PEG4- triazole-P8 | 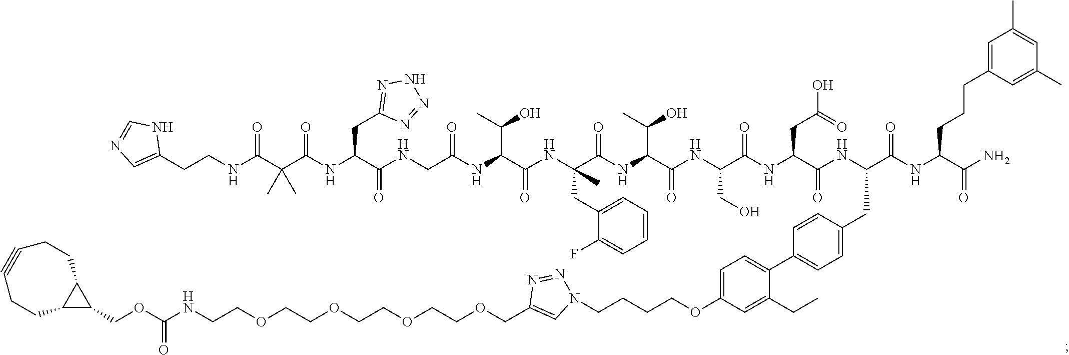 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP10 M3167 | COT- PEG4- triazole-P8 | 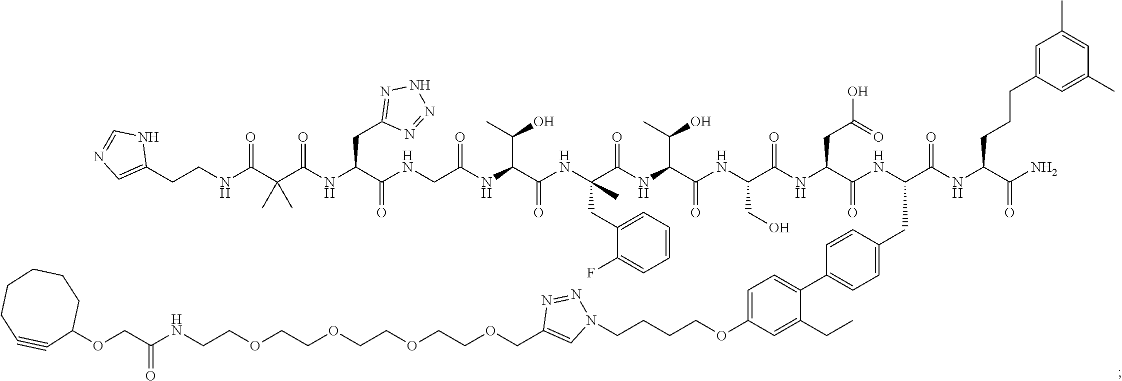 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP11 | NH2- PEG8- triazole-P8 (M3190) | 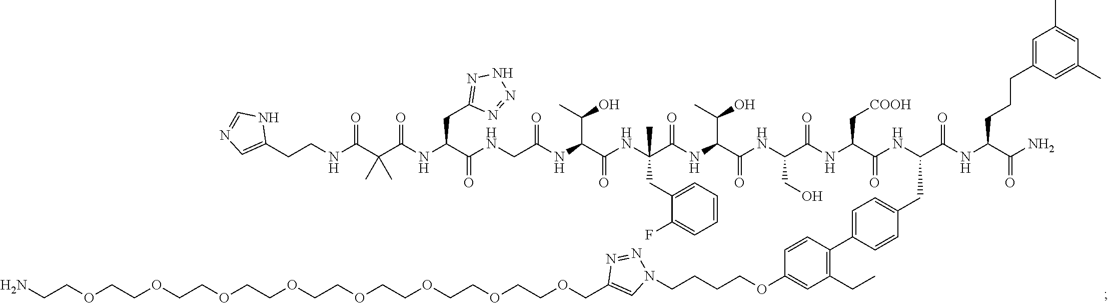 |
| LP12 M2944 | DIBAC- suc- PEG24- P11 | 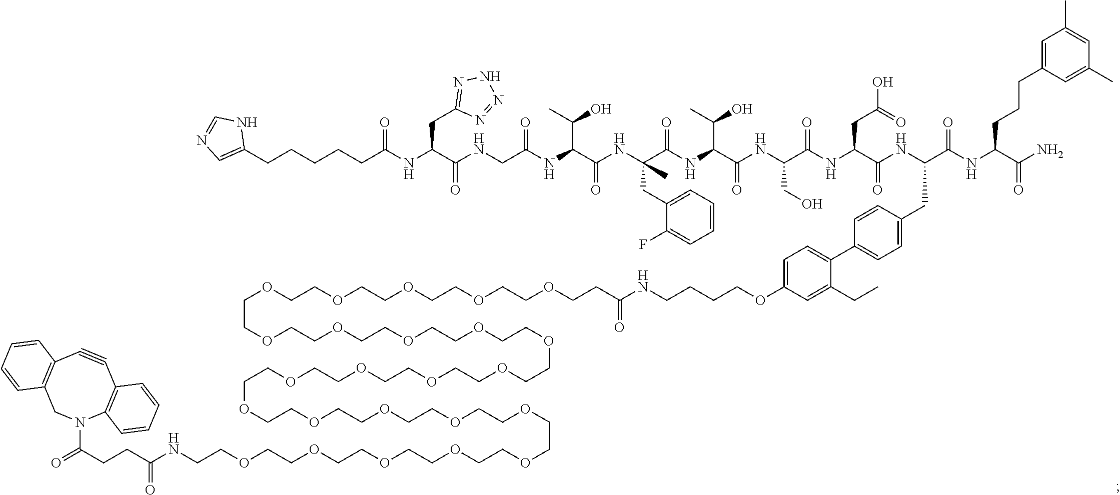 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(N)=O)cc1] |
| LP13 M2876 | DIBAC- suc- PEG24-P4 | 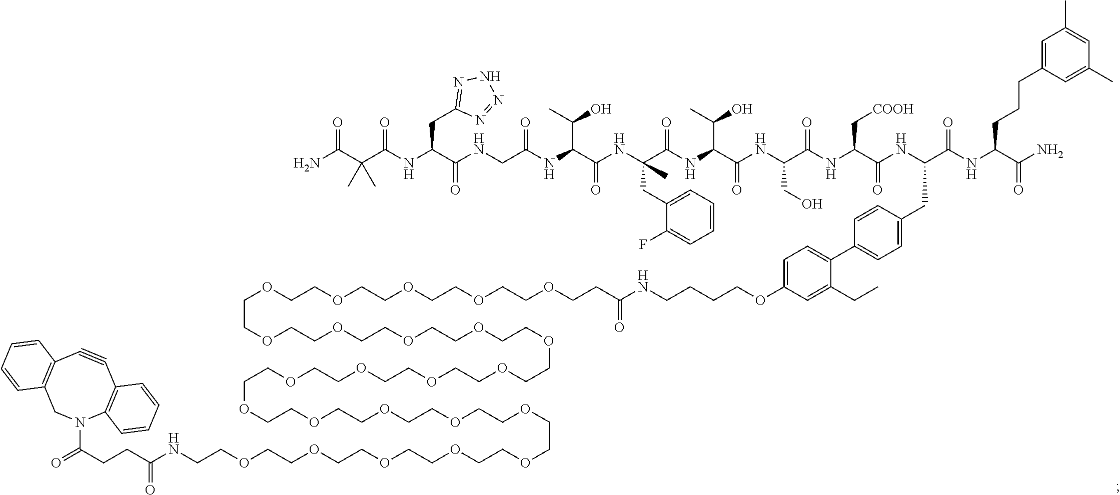 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(N)=O)cc1] |
| LP14 M3055 | DIBAC- suc-G4S- P4 | 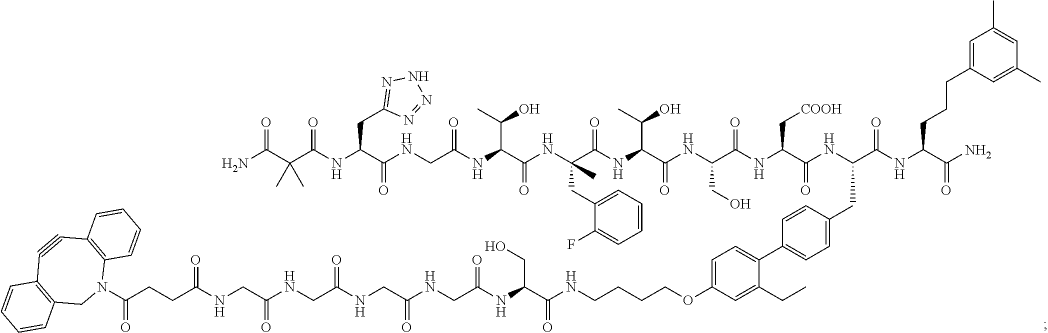 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(N)=O)cc1] |
| LP15 | DIBAC- | [symbol] ; |
| M2964 | suc-G4S- | |
| | P23 | |
| LP16 | DIBAC- | [symbol] ; |
| | suc-SG4- | |
| | P23 | |
| LP17 | DIBAC- | [symbol] ; |
| M2877 | suc-G₄S- | |
| | G₄S-P9 | |
| LP17A M2945 | | 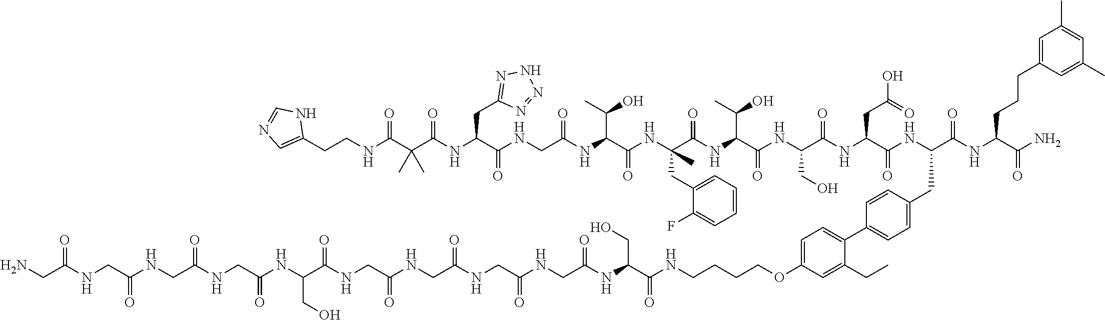 [SMILES: CCc1cc(OCCCCNC(=O)[C@H](CO)NC(=O)CNC(=O)CNC(=O)CNC(=O)CNC(=O)C(CO)NC(=O)CNC(=O)CNC(=O)CNC(=O)CN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP18 M3120 | BCN NHC₂ H₄CO (glucose) SG₄-P9 | 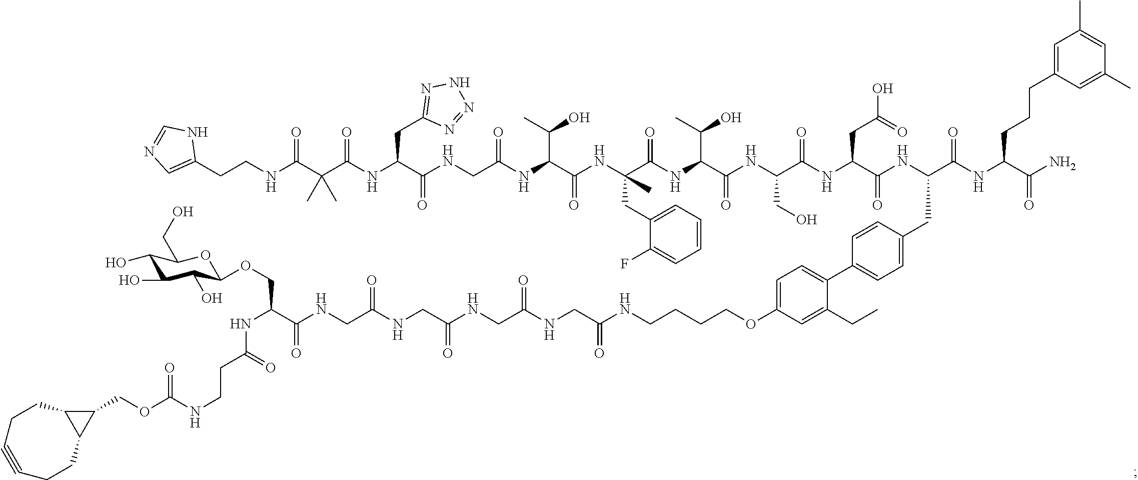 |
| LP19 | COT- G₄- (R)Ser-P9 | 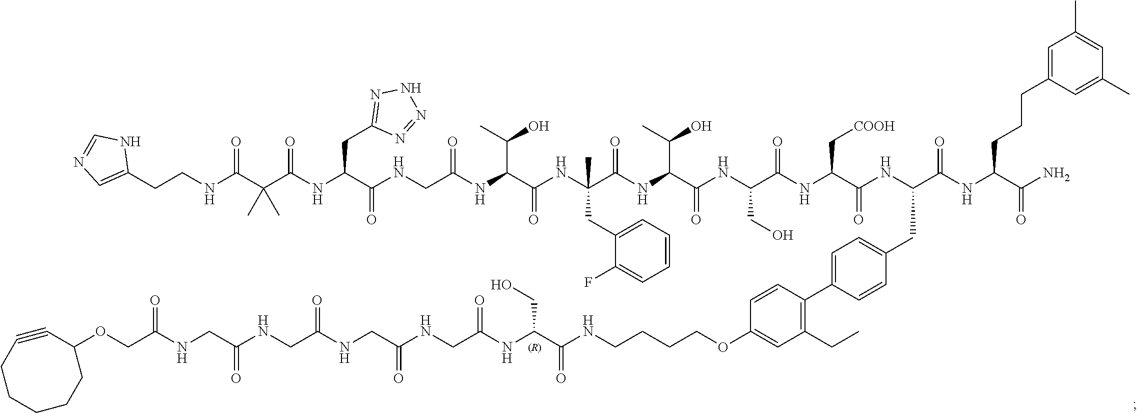 |
| LP20 | DIBAC- suc- (glucose) SG₄-P9 | 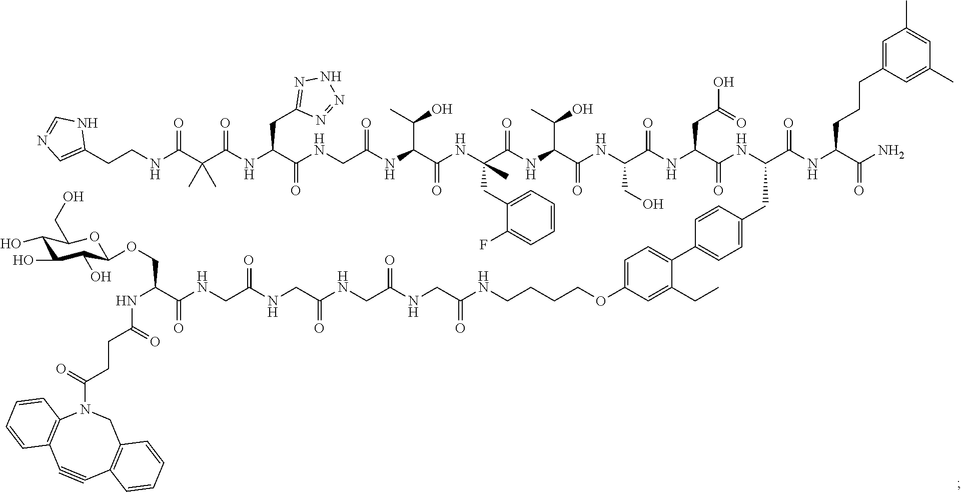 |
| LP21 | DIBAC- suc- PEG24- P24 | 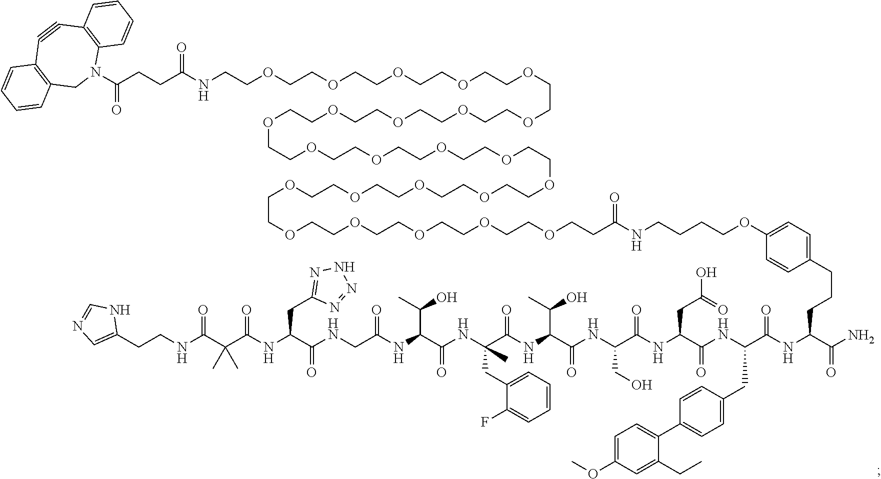 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4C#Cc4ccccc43)cc2)C(N)=O)cc1] |
| LP22 | cyclo- dex- trin- triazole- DIBAC- suc- PEG24-P9 | 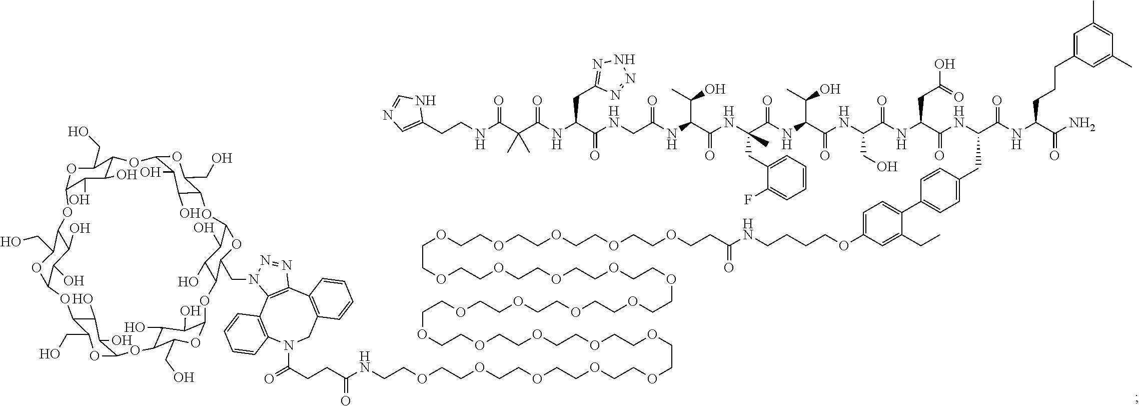 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP23 | cyclo- dex- trin- triazole- DIBAC- suc- PEG24- P24 | 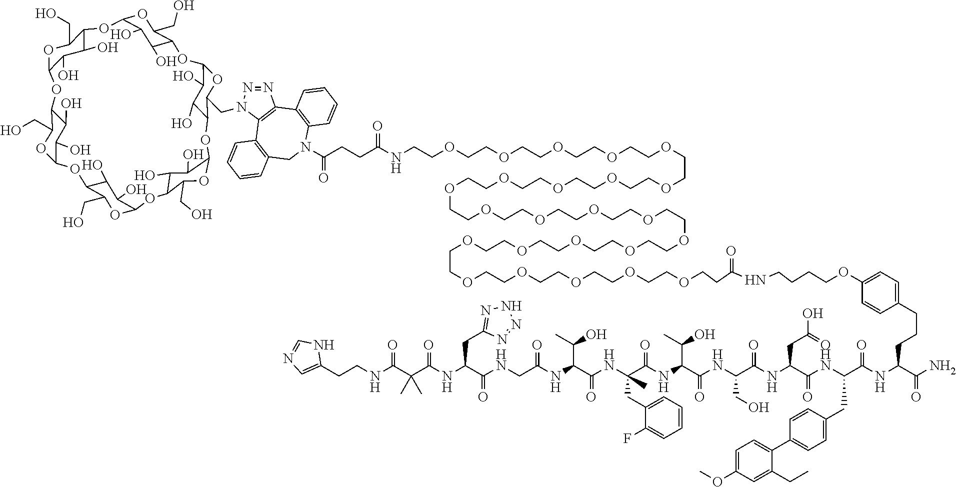 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4-c4c(nnn4CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)-c4ccccc43)cc2)C(N)=O)cc1] |
| LP24 | triazole- BCN- PEG4- triazole- P8 | 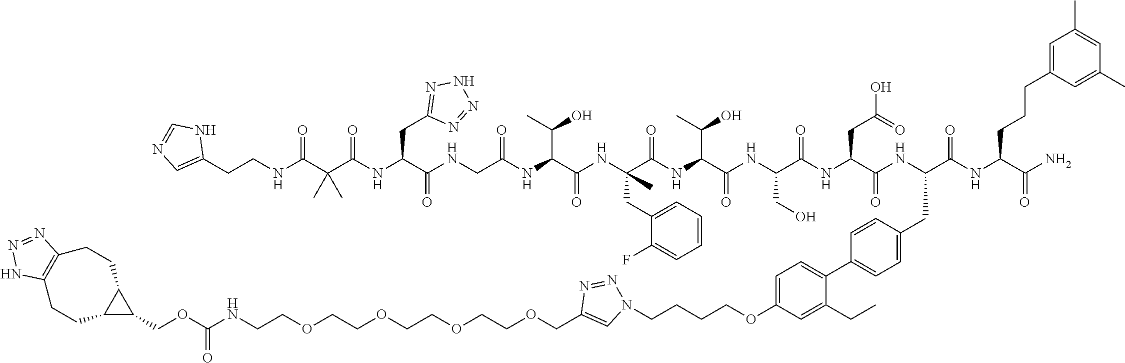 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP25 | triazole- BCN- PEG4- carba- mate- P9 | 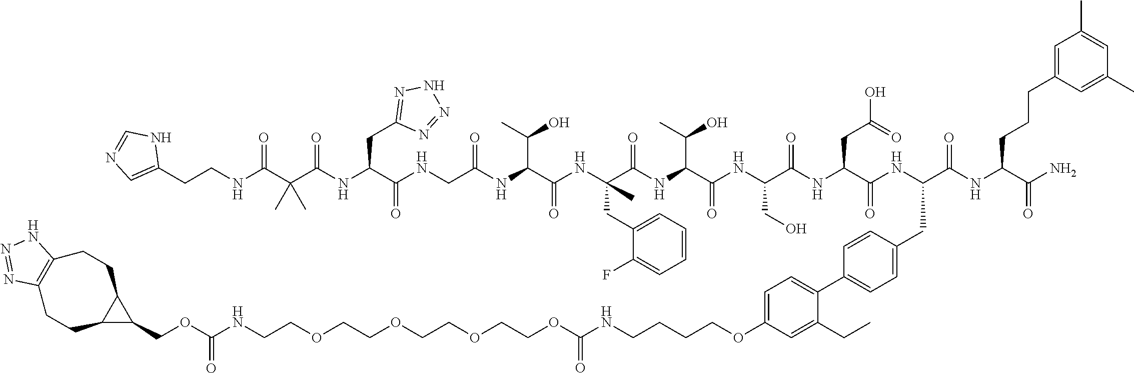 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP26 | triazole- DIBAC- G4S-P9 | 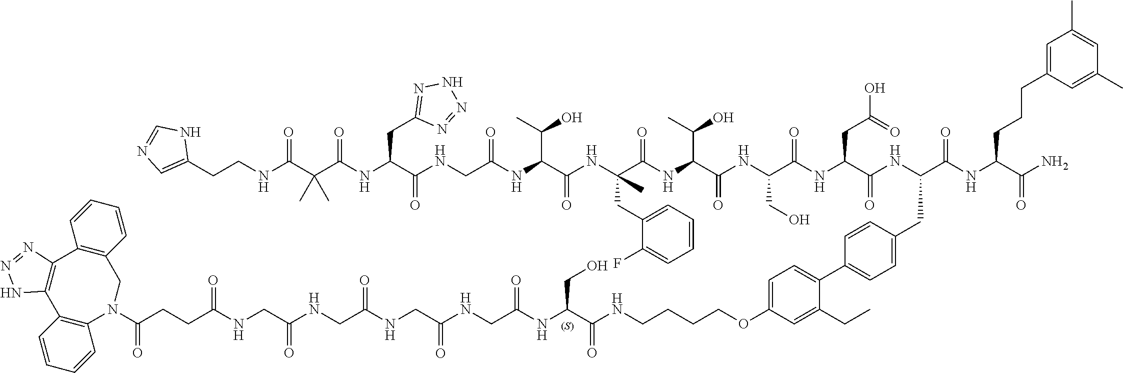 |
| LP27 | DIBAC- suc-PEG8- triazole-P8 | 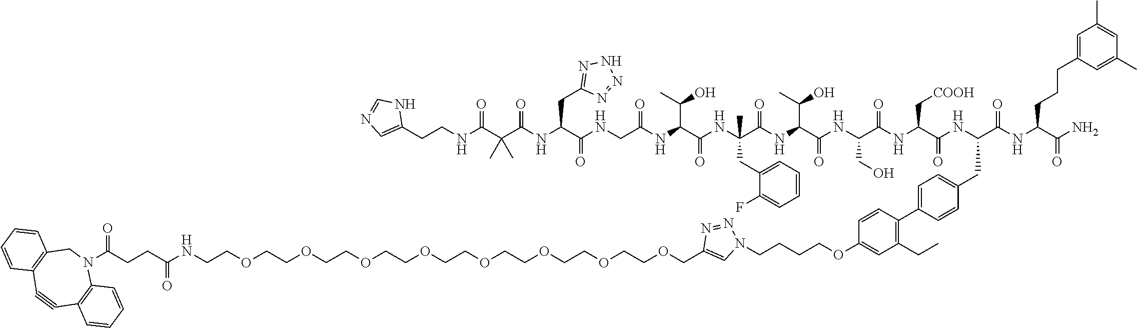 |
| LP28 | triazole- DIBAC- suc-PEG8- triazole-P8 | 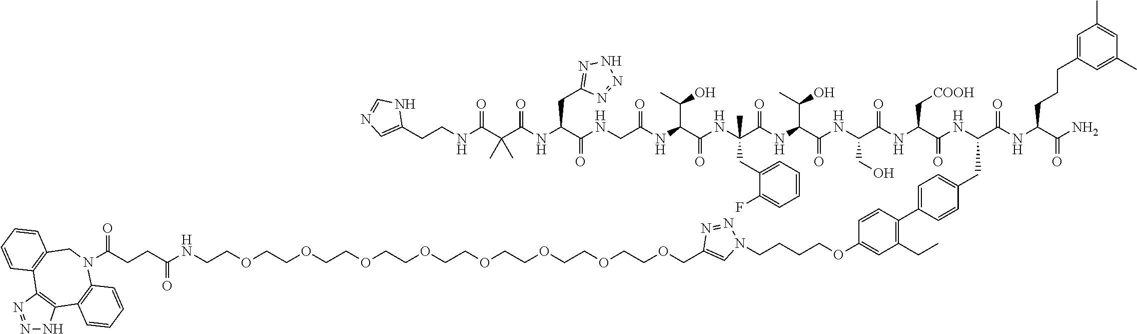 |
| LP29 | NH2- PEG8- triazole- P19 | 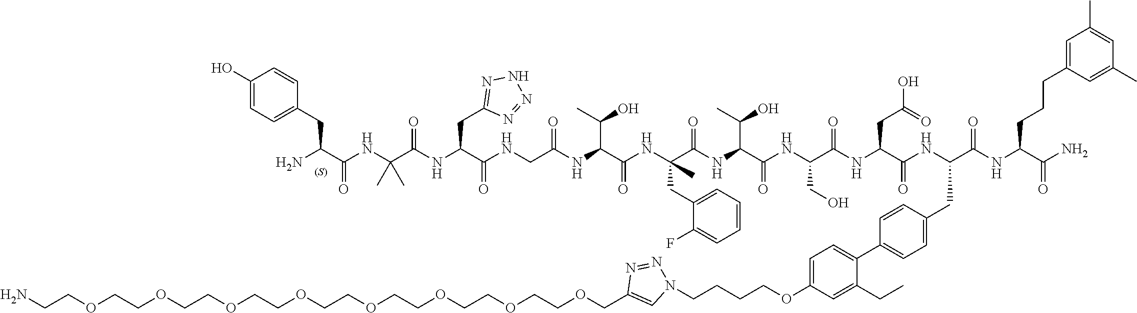 |
| LP30 | NH2- PEG8- triazole- P35 | 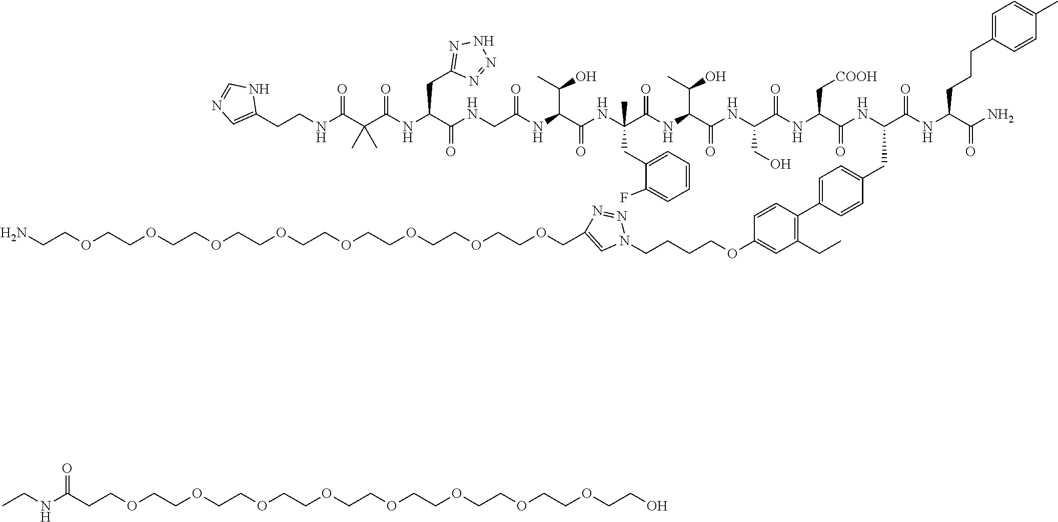 |
| LP31 | NH2- PEG8- triazole- P8 | 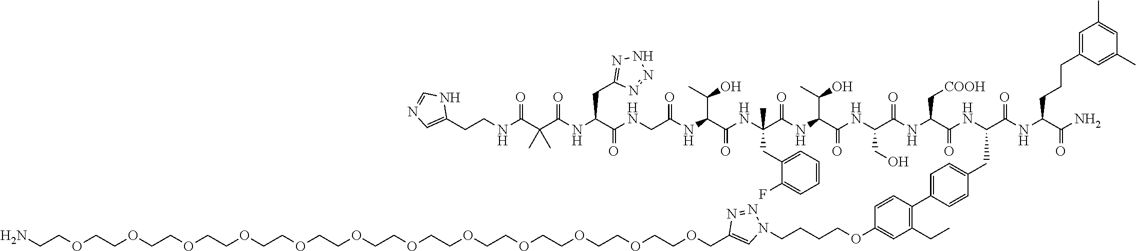 |
| LP32 | NH2- PEG8- triazole- P8 acid | 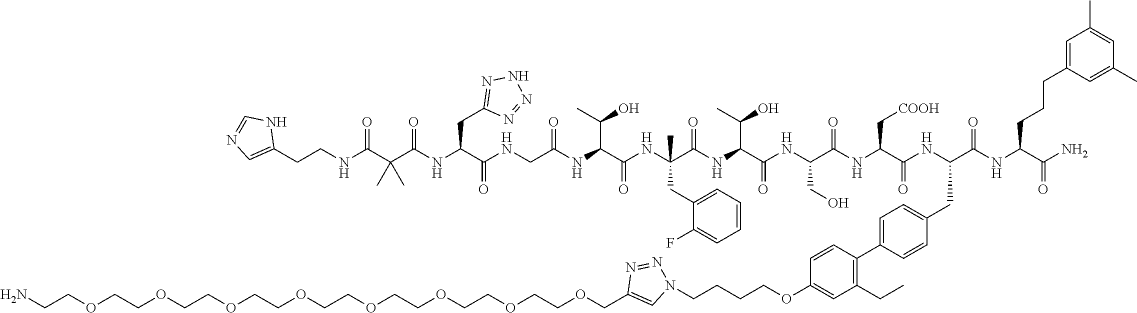 |
| LP33 | E-PEG8- triazole- P8 | 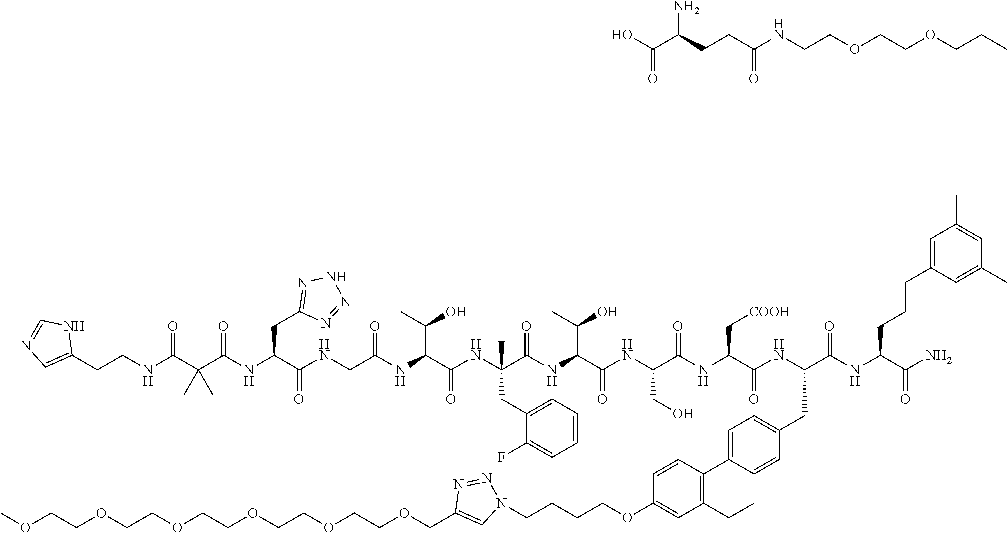 |
| LP34 | GGT EPL- PEG8- triazole- P8 | 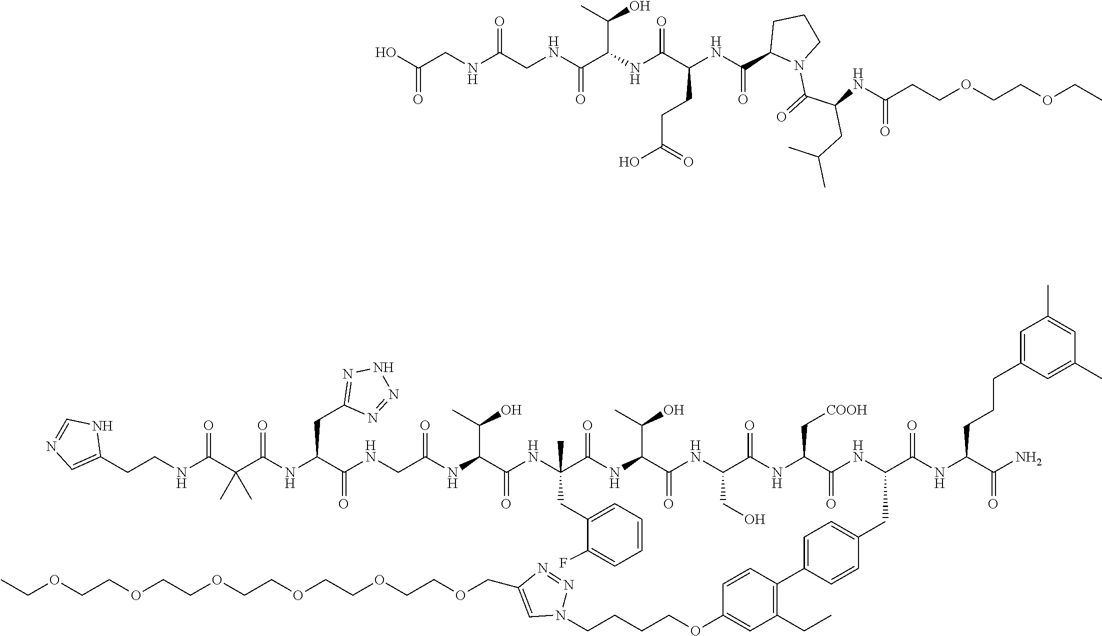 |
| LP35 | Cbz- LLQGSG- PEG8- triazole- P8 | 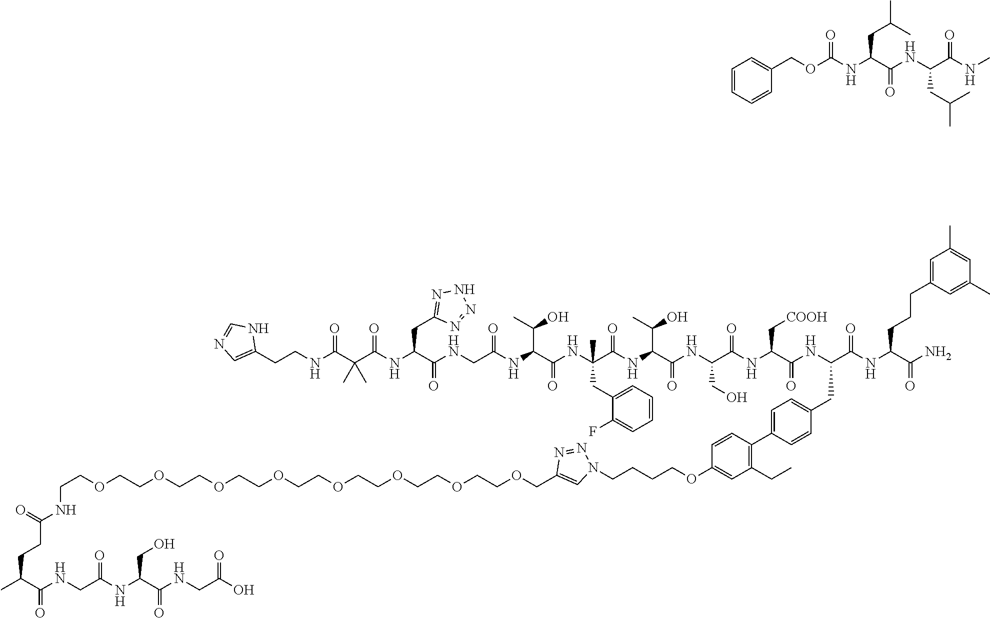 |
| LP36 | G4S triazole- P40 | 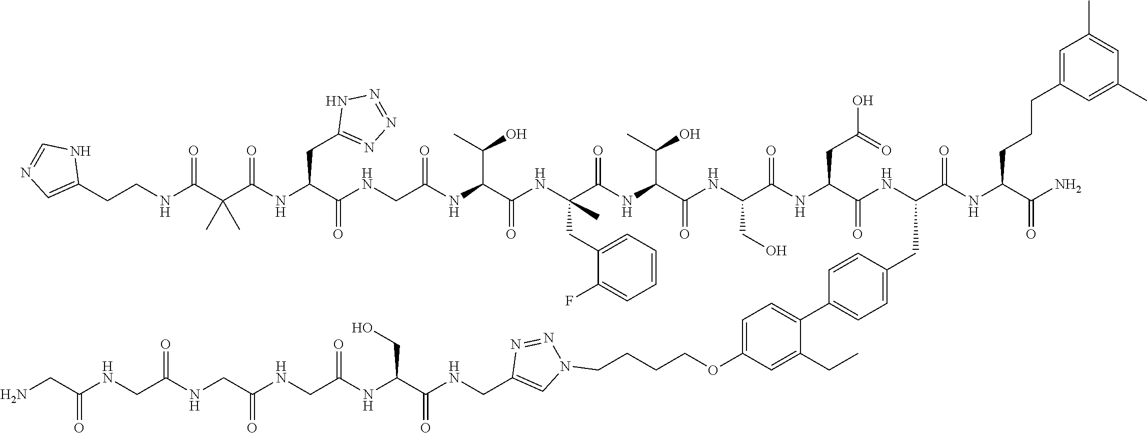 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP37 | SG4- triazole- P40 | 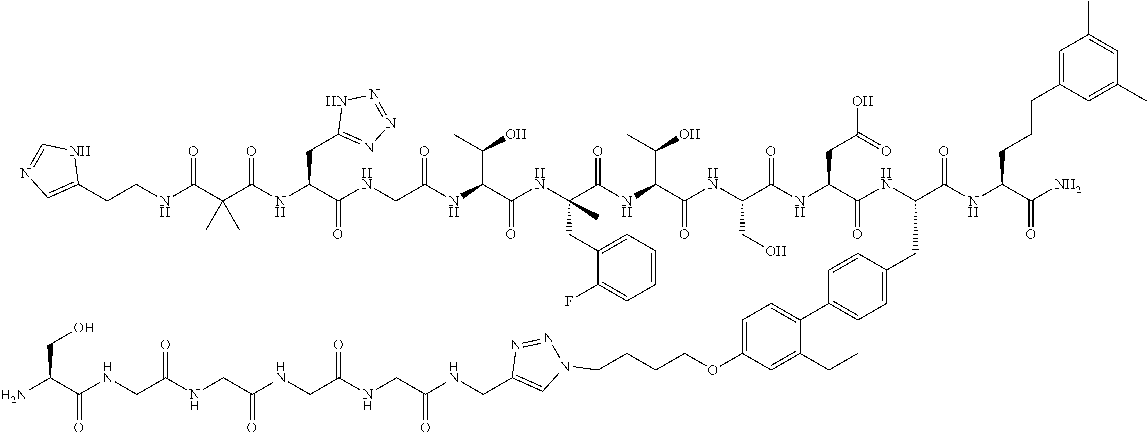 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] |
| LP38 | G2SG2 SG2 triazole- P40 | 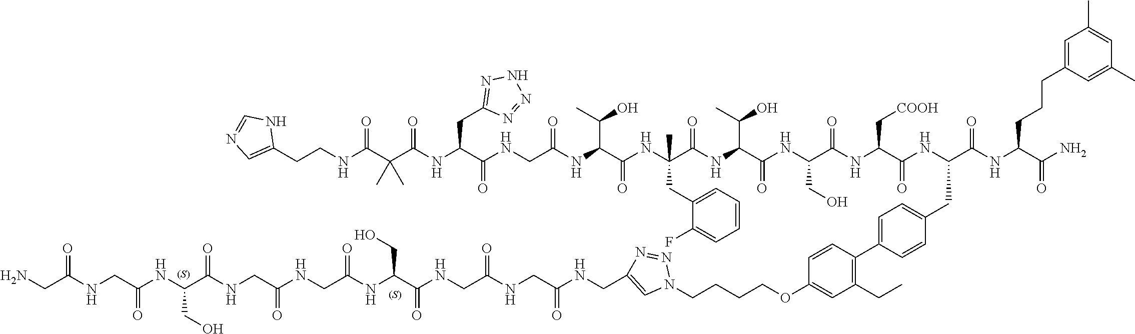 |
| LP39 | G4SG4 triazole- P40 | 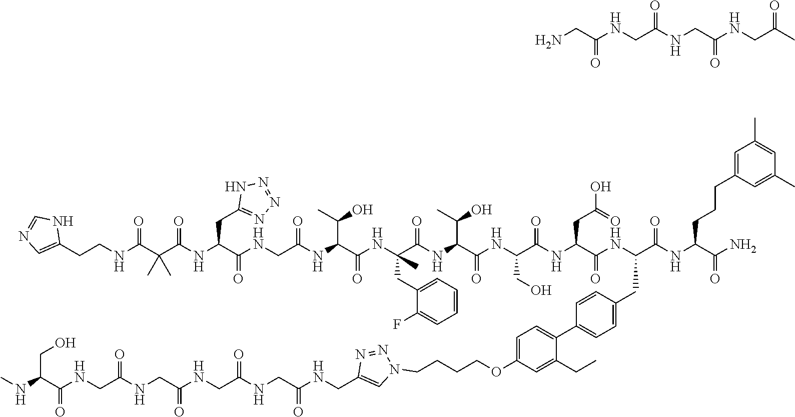 |
| LP40 | G4SG4 SG4 triazole- P40 | 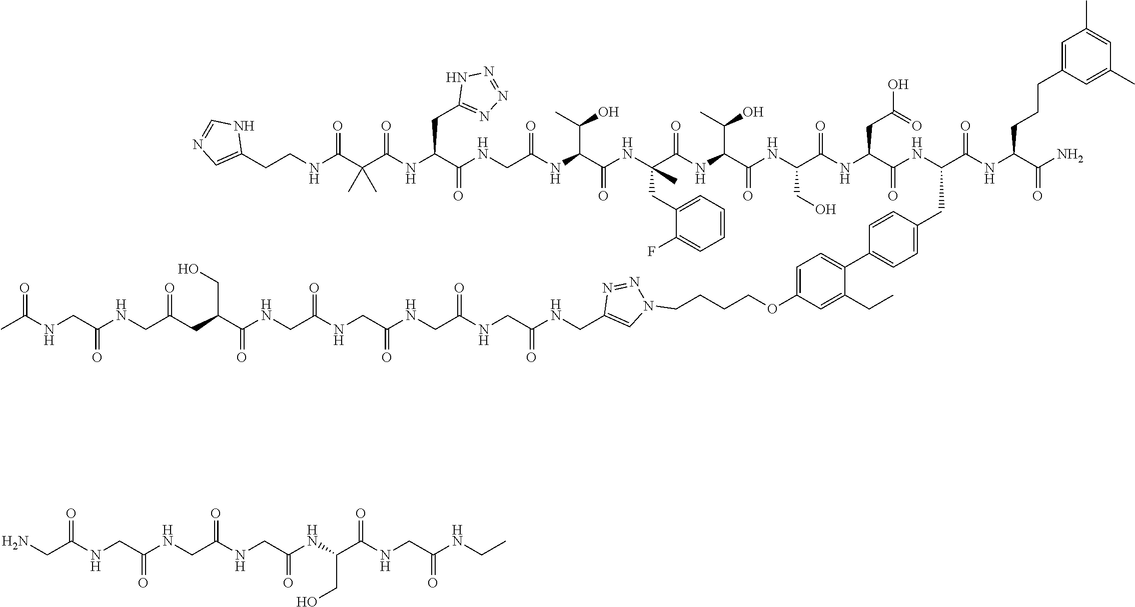 [SMILES: CCc1cc(OCCCCn2cc(CNC(=O)CNC(=O)CNC(=O)CNC(=O)CNC(=O)[C@H](CO)CC(=O)CNC(=O)CNC(C)=O)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1 ; CCNC(=O)CNC(=O)[C@H](CO)NC(=O)CNC(=O)CNC(=O)CNC(=O)CN] |
| LP41 | G2SG2S G2SG2 triazole- P40 | 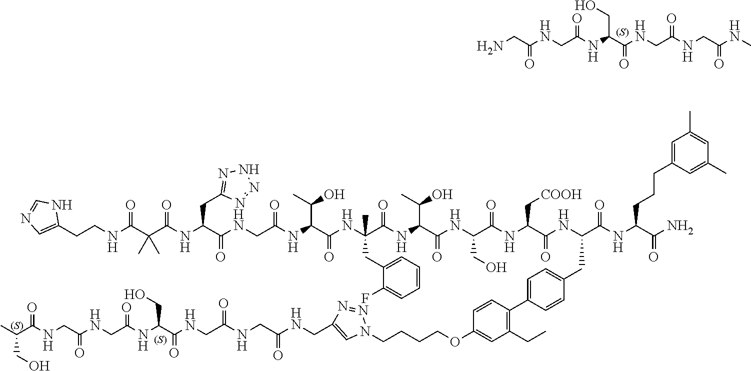 |
| LP42 | C18- diacid- Glu- (AEEA) 2- G4SG4- triazole- P40 | 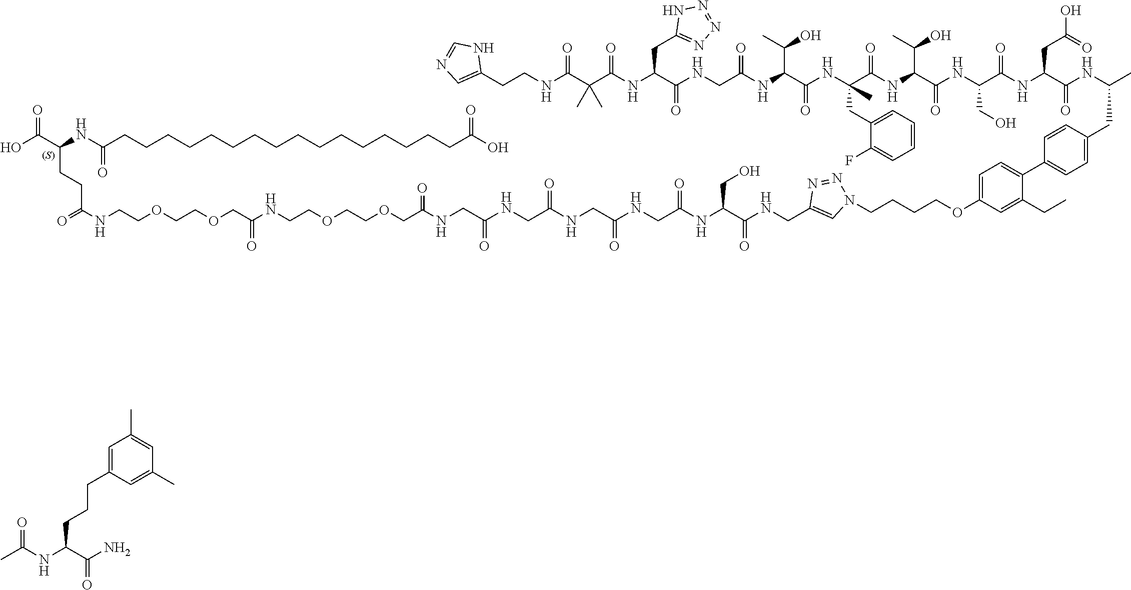 |
| LP43 | C18- diacid- Glu- (AEEA)2- G4SG4- triazole- P40 | 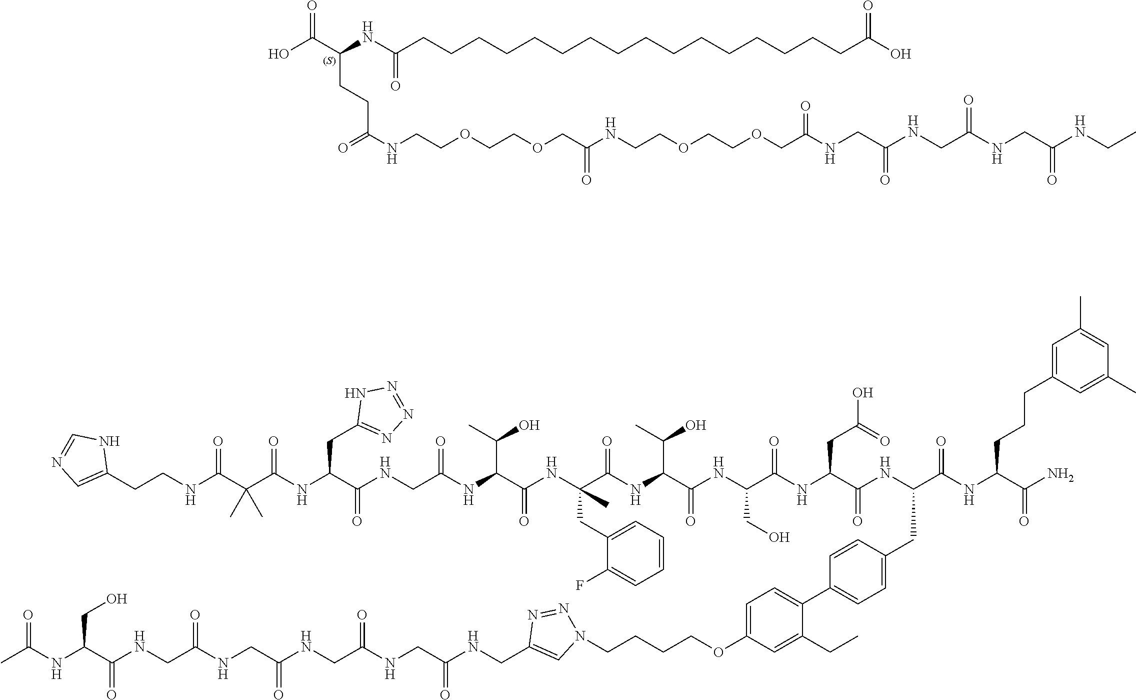 |
| LP44 | C18- diacid- Glu- (AEEA)2- NH2- PEG12- P40 | 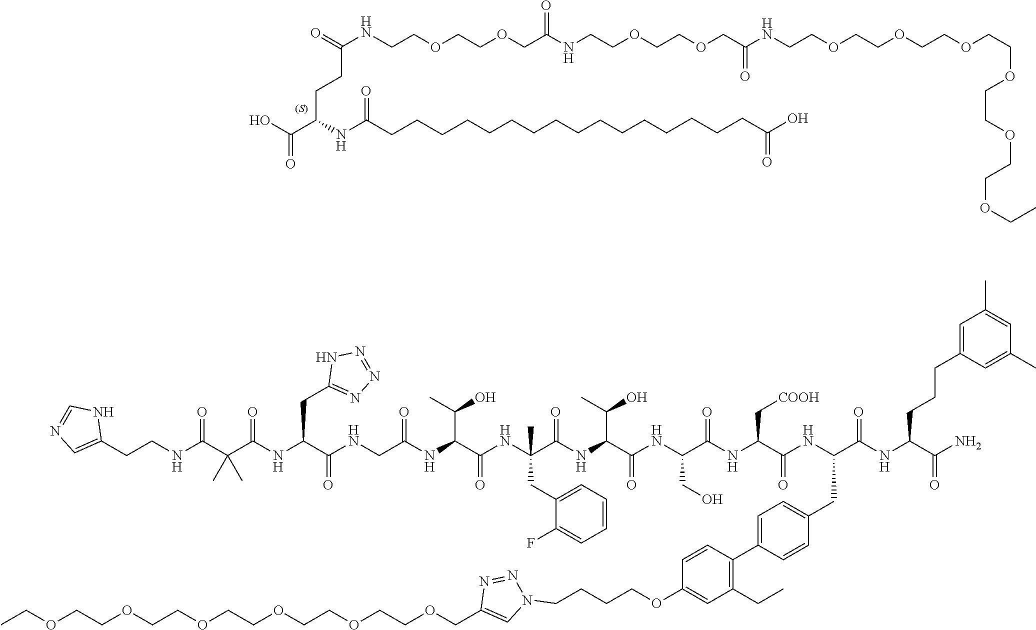 |
| LP45 | C18- diacid- Glu- (AEEA)2- NH2- PEG8- P35 | 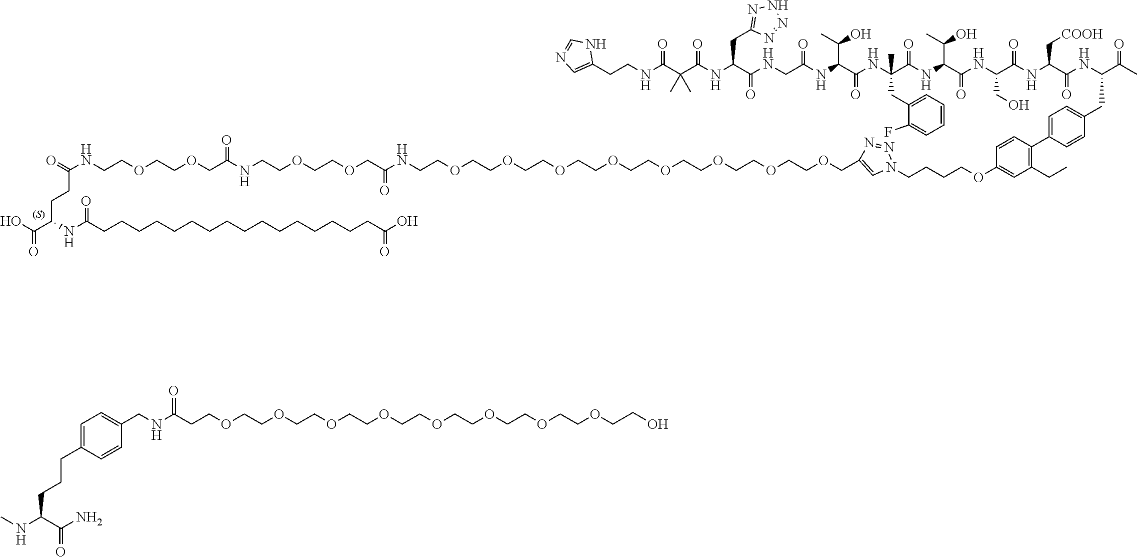 |
| M2547 | | 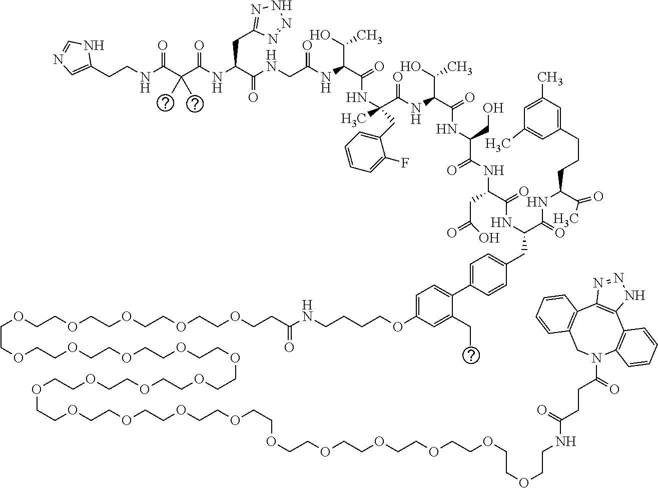 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nn[nH]c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(C)=O)cc1] |

[symbol]

indicates data missing or illegible when filed

In one embodiment of the invention, the linker-payloads as described above have the following properties:

| | | | | RT on | | | |
|---|---|---|---|---|---|---|---|
| | Molecular | | M/Z 100% | HPLC | | | Corresponding |
| LP# | Formula | MW | (M + H) | (min) | | CLogP | Payload |
| LP1 | C₁₀₅H₁₃₅FN₂₀O₂₄ | 2080.31 | 1041 [M + 2H]²⁺ | 15.16 | (A) | 5.55 ± 1.08 | P9 |
| LP2 | C₁₁₃H₁₅₁FN₂₀O₂₈ | 2256.52 | 1129.2 [M + 2H]²⁺ | 15.27 | (A) | 4.12 ± 1.10 | P9 |
| | | | 753.0 [M + 3H]³⁺ | | | | |
| LP3 | C₁₂₁H₁₆₇FN₂₀O₃₂ | 2432.73 | 811.8 [M + 3H]³⁺ | 15.12 | (A) | 2.68 ± 1.12 | P9 |
| | | | 1217.2 [M + 2H]²⁺ | | | | |
| LP4 | C₁₄₅H₂₁₅FN₂₀O₄₄ | 2961.36 | 741.3 [M + 4H]⁴⁺ | 14.76 | (A) | −1.61 ± 1.17 | P9 |
| | | | 988.3 [M + 3H]³⁺ | | | | |
| LP5 | C₉₅H₁₃₀FN₁₉O₂₄ | 1941.16 | 971.5 [M + 2H]²⁺ | 10.12 | (D) | 5.48 ± 1.05 | P9 |
| LP6 | C₁₀₅H₁₃₁FN₂₄O₂₅ | 2148.31 | 716.9 [M + 3H]³⁺ | 3.58 | (E) | 4.24 ± 1.10 | P9 |
| LP7 | C₁₀₅H₁₃₁FN₂₄O₂₅ | 2148.31 | 1074.5 [M + 2H]²⁺ | 3.55 | (E) | 4.24 ± 1.10 | P9 |
| LP8 | C₁₀₅H₁₃₃FN₂₂O₂₃ | 2090.31 | 1045.5 [M + 2H]²⁺ | 2.86 | (E) | 5.95 ± 1.47 | P8 |
| LP9 | C₉₇H₁₃₂FN₂₁O₂₃ | 1979.21 | 990.5 [M + 2H]²⁺ | 9.89 | (D) | 4.59 ± 1.44 | P8 |
| | | | 660.7 [M + 3H]³⁺ | | | | |
| LP10 | C₉₆H₁₃₂FN₂₁O₂₃ | 1967.2 | 984.7 [M + 2H]²⁺ | 9.74 | (D) | 7.75 ± 1.55 | P8 |
| LP11 | C₉₄H₁₃₆FN₂₁O₂₅•2(CF₃COOH) | 2207.26 | 990.6 [M + 2H]²⁺ | 9.40 | (B) | 0.52 ± 1.46 | P8 |
| | | | 660.8 [M + 3H]³⁺ | | | | |
| | | | 495.8 [M + 4H]⁴⁺ | | | | |
| LP12 | C₁₄₄H₂₁₄FN₁₉O₄₃ | 2918.34 | 974 [M + 3H]³⁺ | 2.53 | (E) | −0.14 ± 1.16 | P11 |
| | | | 1459.6 [M + 2H]²⁺ | | | | |
| LP13 | C₁₄₀H₂₀₉FN₁₈O₄₄ | 2867.25 | 717.8 [M + 4H]⁴⁺ | 3.97 | (E) | −1.59 ± 1.16 | P4 |
| | | | 956.9 [M + 3H]³⁺ | | | | |
| | | | 1434.7 [M + 2H]²⁺ | | | | |
| LP14 | C₁₀₀H₁₂₅FN₂₂O₂₅ | 2054.19 | 1028.5 [M + 2H]²⁺ | 3.57 | (E) | 4.26 ± 1.09 | P4 |
| LP15 | C₁₁₁H₁₃₅FN₂₄O₂₅ | 2224.4 | 1113.1 [M + 2H]²⁺ | 2.76 | (E) | 6.67 ± 1.09 | P23 |
| LP16 | C₁₁H₁₃₅FN₂₄O₂₅ | 2224.4 | 1112.9 [M + 2H]²⁺ | 3.73 | (E) | 6.67 ± 1.09 | P23 |
| LP17 | C₁₁₆H₁₄₈FN₂₉O₃₁ | 2463.59 | 1232.4 [M + 2H]²⁺ | 2.17 | (E) | 0.93 ± 1.14 | P9 |
| LP18 | C₁₀₆H₁₄₅FN₂₄O₃₁ | 2270.43 | 757.7 [M + 3H]³⁺ | 8.17 | (D) | 0.36 ± 1.09 | P9 |
| | | | 1136.0 [M + 2H]²⁺ | | | | |
| LP19 | C₉₆H₁₃₀FN₂₃O₂₅ | 2025.2 | 1013.7 [M + 2H]²⁺ | 8.70 | (B) | 2.38 ± 1.09 | P9 |
| LP20 | C111H141FN24O30 | 2310.45 | 1156.0 [M + 2H]²⁺ | 2.28 | (E) | 2.54 ± 1.10 | P9 |
| LP21 | C₁₄₄H₂₁₃FN₂₀O₄₅ | 2963.34 | 741.6 [M + 4H]⁴⁺ | 14.49 | (A) | −2.62 ± 1.17 | P24 |
| | | | 988.5 [M + 3H]³⁺ | | | | |
| LP22 | C₁₈₁H₂₇₄FN₂₃O₇₃ | 3959.22 | 1321.0 [M + 3H]³⁺ | 11.97 | (A) | N/A | P9 |
| | | | 991.3 [M + 4H]⁴⁺ | | | | |
| LP23 | C₁₈₀H₂₇₂FN₂₃O₇₄ | 3961.2 | 991.4 [M + 4H]⁴⁺ | 8.14 | (D) | N/A | P24 |
| | | | 1321.5 [M + 3H]³⁺ | | | | |
| LP24 | C₉₇H₁₃₃FN₂₄O₂₃ | 2022.2 | 675.1 [M + 3H]³⁺ | 3.56 | (F) | 2.82 ± 1.45 | P8 |
| | | | 1012.0 [M + 2H]²⁺ | | | | |
| LP25 | C₉₅H₁₃₁FN₂₂O₂₄ | 1984.2 | 662.4 [M + 3H]³⁺ | 3.57 | (F) | 3.71 ± 1.43 | P9 |
| | | | 1012.0 [M + 2H]²⁺ | | | | |
| LP26 | C₁₀₅H₁₃₂FN₂₇O₂₅ | 2191.3 | 1096.7 [M + 2H]²⁺ | 3.26 | (F) | 2.54 ± 1.53 | P9 |
| LP27 | C113H149FN22O27 | 2266.5 | 756.03 [M + 3H]³⁺ | 9.17 | (E) | 4.52 ± 1.49 | P8 |
| LP28 | C113H150FN25O27• | 2423.57 | 2309.11 [M + H]⁺ | 4.20 | (F) | 3.81 +/− 1.50 | P8 |
| | | | 1155.56 [M + 2H]²⁺ | | | | |
| LP29 | C₉₇H₁₃₉FN₂0O₂₆• | 2248.3 | 1011.0 [M + 2H]²⁺ | 3.73 | (F) | 1.90 ± 1.46 | P19 |
| LP30 | C₁₁₂H₁₇₁FN₂₂O₃₅• | 2632.7 | 802.20 [M + 3H]³⁺ | 6.32 | (D) | −5.09 ± 1.51 | P35 |
| LP31 | C₁₀₂H₁₅₂FN₂₁O₂₉• | 2383.5 | 1078.6 [M + 2H]²⁺ | 7.48 | (D) | −0.92 ± 1.48 | P8 |
| LP32 | C₉₄H₁₃₅FN₂₀O₂₆• | 2208.24 | 991.0 [M + 2H]²⁺ | 7.56 | (D) | 1.49 +/− 1.46 | P41 |
| LP33 | C₉₉H₁₄₃FN₂₂O₂₈ | 2108.32 | 1054.9 [M + 2H]²⁺ | 3.27 | (E) | −0.23 +/− 1.49 | P8 |
| LP34 | C₁₁₉H₁₇₃FN₂₆O₃₆• | 2659.81 | 1282.6 [M + 2H]²⁺ | 4.34 | (E) | −0.48 +/− 1.56 | P8 |
| LP35 | C₁₂₆H₁₈₂FN₂₇O₃₆• | 2783.97 | 1335.7 [M + 2H]²⁺ | 7.15 | (D) | 2.72 +/− 1.54 | P8 |
| | | | 890.9 [M + 3H]³⁺ | | | | |
| | | | 668.4 [M + 4H]⁴⁺ | | | | |
| LP36 | C89H121FN26O23• | 2170.12 | 971.9 [M + 2H]²⁺ | 6.62 | (D) | −0.54 +/− 1.47 | P40 |
| LP37 | C₈₉H₁₂₁FN₂₆O₂₃• | 2170.12 | 971.0 [M + 2H]²⁺ | 6.61 | (D) | −0.61 +/− 1.47 | P40 |
| LP38 | C₉₆H₁₃₂FN₂₉O₂₇ | 2143.25 | 1072.30[M + 2H]²⁺ | 8.96 | (D) | −2.88 +/− 1.49 | P40 |
| | | | 715.30 [M + 3H]³⁺ | | | | |
| LP39 | C₉₇H₁₃₃FN₃₀O₂₇ | 2170.28 | 1085.60 [M + 2H]²⁺ | 8.97 | (D) | −3.00 +/− 1.50 | P40 |
| | | | 725.30 [M + 3H]³⁺ | | | | |
| LP40 | C₁₀₈H₁₅₀FN₃₅O₃₃ | 2485.56 | 1243.05 [M + 2H]²⁺ | 6.50 | (D) | −6.31 +/− 1.55 | P40 |
| | | | 829.03 [M + 3H]³⁺ | | | | |
| LP41 | C₁₀₃H₁₄₃FN₃₂O₃₁ | 2344.43 | 1173.21 [M + 2H]²⁺ | 6.54 | (D) | −4.96 +/− 1.52 | P40 |
| | | | 782.47 [M + 3H]³⁺ | | | | |
| LP42 | C₁₂₄H₁₈₂FN₂₉O₃₅• | 2657.94 | 1329.17 [M + 2H]²⁺ | 8.53 | (D) | 3.04 +/− 1.54 | P40 |
| LP43 | C₁₃₂H₁₉₄FN₃₃O₃₉• | 2886.15 | 1443.21 [M + 2H]2+ | 8.30 | (D) | 0.58 +/− 1.57 | P40 |
| LP44 | C₁₃₇H₂₁₃FN₂₄O₄₁• | 2985.32 | 1436.4 [M + 2H]²⁺ | 9.30 | (D) | 2.56 +/− 1.56 | P40 |
| LP45 | C₁₄₇H₂₃₂FN₂₅O₄₇• | 3120.55 | 781.1 [M + 4H]⁴⁺ | 8.14 | (D) | −1.88 +/− 1.58 | P35 |

In the present disclosure, the antibody can be any antibody deemed suitable to the practitioner of skill. In some embodiments of the invention, a linker or linker-payload is attached to one or both heavy chains of the antibody or antigen-binding fragment thereof. In some embodiments, a linker or linker-payload is attached to one or both heavy chain variable domains of the antibody or antigen-binding fragment thereof.

In an embodiment of the invention, a linker or linker-payload is attached to the N-terminus of one or both heavy chain variable domains of the antibody or antigen-binding fragment thereof; the N-terminus of both heavy chain variable domains of the antibody or antigen-binding fragment thereof; one or both light chains of the antibody or antigen-binding fragment thereof; one or both light chain variable domains of the antibody or antigen-binding fragment thereof; the N-terminus of one or both light chain variable domains of the antibody or antigen-binding fragment thereof; the N-terminus of both light chain variable domains of the antibody or antigen-binding fragment thereof; the C-terminus of one or both heavy chain variable domains of the antibody or antigen-binding fragment thereof; the C-terminus of both heavy chain variable domains of the antibody or antigen-binding fragment thereof; the C-terminus of one or both light chain variable domains of the antibody or antigen-binding fragment thereof; and/or the C-terminus of both light chain variable domains of the antibody or antigen-binding fragment thereof.

In an embodiment of the invention, the antibody or antigen-binding fragment comprises at least one glutamine residue in at least one polypeptide chain sequence. In an embodiment of the invention, the antibody or antigen-binding fragment comprises one or more Gln295 residues. Typically, the Gln295 residue is conserved in the heavy chain and in the context of EEQYNS (SEQ ID NO: 439) or EEQFNS (SEQ ID NO: 440). See Mindt, et al., Modification of different IgG1 antibodies via glutamine and lysine using bacterial and human tissue transglutaminase. Bioconjug Chem 2008; 19: 271-8; and Jeger et al., Site-specific and stoichiometric modification of antibodies by bacterial transglutaminase, Angew Chem Int Ed Engl 2010; 49: 9995-7. A “Gln295”, which may be discussed herein, may not be the 295^{th} residue in the heavy chain however. See for example, SEQ ID NO: 42 herein. In an embodiment of the invention, the antibody or antigen-binding fragment comprises two heavy chain polypeptides, each with one Gln295 residue. In an embodiment of the invention, the antibody or antigen-binding fragment comprises one or more glutamine residues at a site other than a heavy chain Gln295. Such antibodies and antigen-binding fragments can be isolated from natural sources or engineered to comprise one or more glutamine residues. Techniques for engineering glutamine residues into an antibody or antigen-binding fragment polypeptide chain are within the skill of the practitioners in the art. In an embodiment of the invention, a glutamine residue is introduced to the N-terminus of an antibody or antigen-binding fragment polypeptide chain. In an embodiment of the invention, a glutamine residue is introduced to the N-terminus of one or both heavy chains of the antibody or antigen-binding fragment. In an embodiment of the invention, a glutamine residue is introduced to the N-terminus of both heavy chains of the antibody or antigen-binding fragment. In an embodiment of the invention, the glutamine residue is introduced to the N-terminus of one or both light chains of the antibody or antigen-binding fragment. In an embodiment of the invention, a glutamine residue is introduced to the N-terminus of both light chains of the antibody or antigen-binding fragment. In an embodiment of the invention, a glutamine residue is introduced to the N-terminus of one or both heavy chains and one or both light chains of the antibody or antigen-binding fragment. In an embodiment of the invention, the Gln295 (Q295) is in the context of an EEQFNS (amino acids 292-297 of SEQ ID NO: 42) motif (“EEQFNS” disclosed as SEQ ID NO: 440).

In an embodiment of the invention, a glutamine residue is introduced to the C-terminus of an antibody or antigen-binding fragment polypeptide chain. In an embodiment of the invention, a glutamine residue is introduced to the C-terminus of one or both heavy chains of the antibody or antigen-binding fragment. In an embodiment of the invention, a glutamine residue is introduced to the C-terminus of both heavy chains of the antibody or antigen-binding fragment. In an embodiment of the invention, the glutamine residue is introduced to the C-terminus of one or both light chains of the antibody or antigen-binding fragment. In an embodiment of the invention, a glutamine residue is introduced to the C-terminus of both light chains of the antibody or antigen-binding fragment. In an embodiment of the invention, a glutamine residue is introduced to the C-terminus of one or both heavy chains and one or both light chains of the antibody or antigen-binding fragment.

Examples of anti-GLP1R antibodies are those disclosed in U.S. Patent Application Publication No. US20060275288A1, which is incorporated herein by reference in its entirety. See also glutazumab. See Li et al., Glutazumab, a novel long-lasting GLP-1/anti-GLP-1R antibody fusion protein, exerts anti-diabetic effects through targeting dual receptor binding sites, Biochem Pharmacol. 2018 April; 150:46-53, Epub 2018 Feb. 3.

In some embodiments of the invention, anti-GLP1R antibodies have a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In one aspect, the present disclosure provides antibody-drug conjugates comprising an anti-GLP1R antibody or antigen-binding fragment thereof as described above and a therapeutic agent (e.g., a GLP1 peptidomimetic). In some embodiments, the antibody or antigen-binding fragment and the payload are covalently attached via a linker, as discussed above. In various embodiments, the anti-GLP1R antibody or antigen-binding fragment can be any of the anti-GLP1R antibodies or fragments described herein. In some embodiments, the ATDCs of the present disclosure are stable in plasma. Plasma stability may be determined using an in vitro or in vivo plasma stablity assay, such as those set forth in Example 8.2 or Example 10 below. In some embodiments, the ATDCs of the present disclosure have a half life of longer than 4 days, longer than 5 days, longer than 6 days, longer than 7 days, longer than 8 days, longer than 9 days, longer than 10 days, longer than 11 days, longer than 12 days, longer than 13 days, longer than 2 weeks, longer than 3 weeks, longer than 4 weeks, about 1 week, about 2 weeks, about 3 weeks, about 4 weeks, about 1 month, about 2 month, about 3 month, about 4 month, about 5 month, about 6 month, between 5-10 days, between 8-12 days, between 10-15 days, between 12-18 days, between 15-20 days, between 20-30 days, between 1-2 weeks, between 2-3 weeks, between 3-4 weeks, between 4-6 weeks, between 5-8 weeks, between 6-10 weeks, between 1-2 months, between 1.5-3 months, between 2-4 months, between 2.5-5 months, between 3-6 months, or between 4-6 months in plasma.

In some embodiments, the ATDCs of the present disclosure bind to GLP1R with at least a 10-fold greater affinity than other G protein-coupled receptors (GPCRs). In some embodiments of the invention, the ATDCs of the present disclosure bind to GLP1R with at least a 20-fold, 50-fold, 100-fold, 500-fold, 1000-fold, 10,000-fold greater affinity than other G protein-coupled receptors (GPCRs). In some embodiments, such ATDCs exhibit essentially undetectable binding against GPCRs other than GLP1R. Binding of the ATDCs to a target molecule can be measured using a standard binding assay available in the relevant art, such as luciferase reporter assay, surface plasmon resonance assay, enzyme-linked immunosorbent assay (ELISA), radioimmunoassay (RIA), FACS analysis, or Western blot assay. Examples of GPCRs other than GLP1R include, but are not limited to, GIPR, GLP2R and GCGR.

In certain embodiments, an ATDC of the present disclosure comprises an anti-GLP1R antibody or antigen-binding fragment thereof, conjugated with a linker payload, wherein the linker payload is attached to the antibody, or antigen-binding fragment thereof, at the N-terminus of a light chain. In one embodiment, an antibody drug conjugate of the present disclosure comprises an anti-GLP1R antibody or antigen-binding fragment thereof conjugated at the N-terminus of a light chain with a linker payload, wherein the payload has the following structure disclosed as SEQ ID NO: 538:

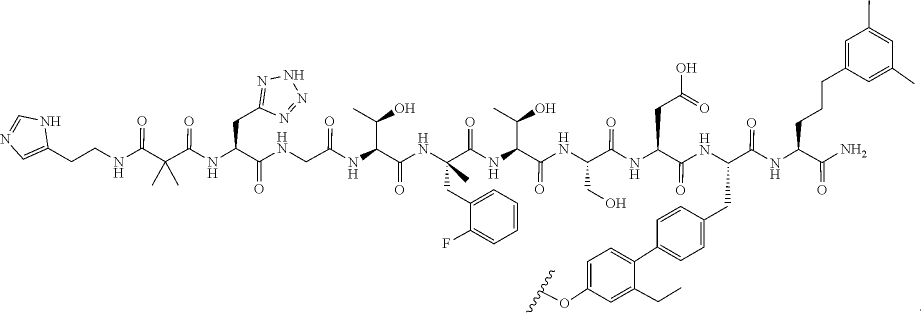 [SMILES: *Oc1ccc(-c2ccc(C[C@H](NC(=O)[C@H](CC(=O)O)NC(=O)[C@H](CO)NC(=O)[C@@H](NC(=O)[C@](C)(Cc3ccccc3F)NC(=O)[C@@H](NC(=O)CNC(=O)[C@H](Cc3nn[nH]n3)NC(=O)C(C)(C)C(=O)NCCc3cnc[nH]3)[C@@H](C)O)[C@@H](C)O)C(=O)N[C@@H](CCCc3cc(C)cc(C)c3)C(N)=O)cc2)c(CC)c1]

In certain embodiments, an ATDC of the present disclosure comprises an anti-GLP1R antibody or antigen-binding fragment thereof conjugated with two linker payloads, wherein each linker payload is attached to the antibody or antigen-binding fragment thereof at the N-terminus of a light chain. In one embodiment, an ATDC of the present disclosure comprises an anti-GLP1R antibody or antigen-binding fragment thereof conjugated at the N-terminus of each light chain with a linker payload for a total of two linker payloads per each antibody, wherein the payload has the following structure disclosed as SEQ ID NO: 538:

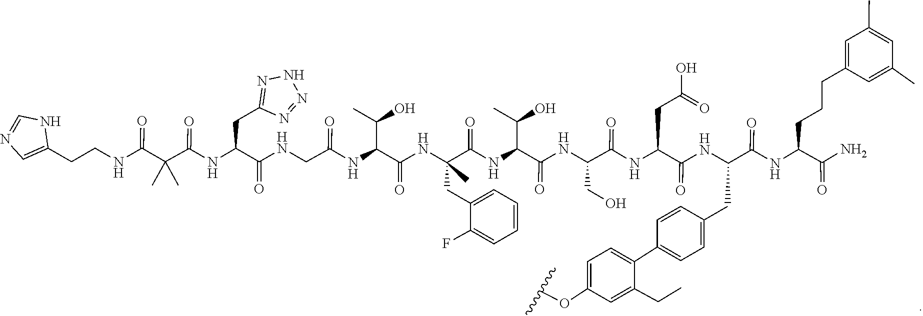 [SMILES: *Oc1ccc(-c2ccc(C[C@H](NC(=O)[C@H](CC(=O)O)NC(=O)[C@H](CO)NC(=O)[C@@H](NC(=O)[C@](C)(Cc3ccccc3F)NC(=O)[C@@H](NC(=O)CNC(=O)[C@H](Cc3nn[nH]n3)NC(=O)C(C)(C)C(=O)NCCc3cnc[nH]3)[C@@H](C)O)[C@@H](C)O)C(=O)N[C@@H](CCCc3cc(C)cc(C)c3)C(N)=O)cc2)c(CC)c1]

In yet another aspect, provided herein is an ATDC comprising a Glucagon-like peptide-1 receptor (GLP1R)-targeting antibody or an antigen-binding fragment thereof and a payload having the structure disclosed as SEQ ID NO: 519:

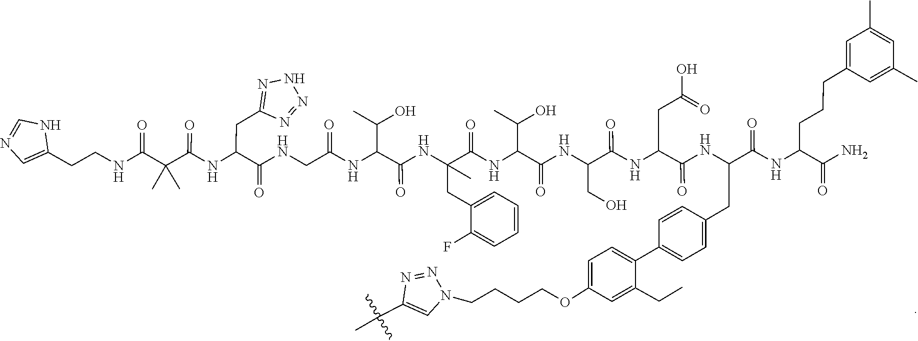 [SMILES: *c1cn(CCCCOc2ccc(-c3ccc(CC(NC(=O)C(CC(=O)O)NC(=O)C(CO)NC(=O)C(NC(=O)C(C)(Cc4ccccc4F)NC(=O)C(NC(=O)CNC(=O)C(Cc4nn[nH]n4)NC(=O)C(C)(C)C(=O)NCCc4cnc[nH]4)C(C)O)C(C)O)C(=O)NC(CCCc4cc(C)cc(C)c4)C(N)=O)cc3)c(CC)c2)nn1]

wherein

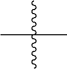

is the point of attachment of the payload to the antibody or the antigen-binding fragment thereof directly or through a linker.

In one embodiment, the payload has the structure disclosed as SEQ ID NO: 519:

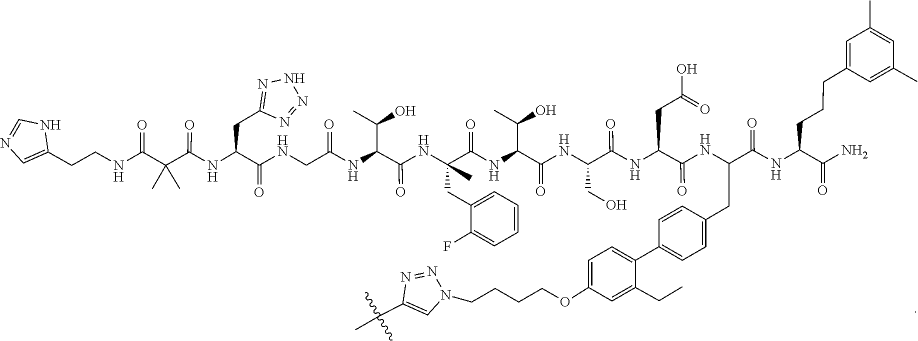 [SMILES: *c1cn(CCCCOc2ccc(-c3ccc(CC(NC(=O)[C@H](CC(=O)O)NC(=O)[C@H](CO)NC(=O)[C@@H](NC(=O)[C@](C)(Cc4ccccc4F)NC(=O)[C@@H](NC(=O)CNC(=O)[C@H](Cc4nn[nH]n4)NC(=O)C(C)(C)C(=O)NCCc4cnc[nH]4)[C@@H](C)O)[C@@H](C)O)C(=O)N[C@@H](CCCc4cc(C)cc(C)c4)C(N)=O)cc3)c(CC)c2)nn1]

In yet another aspect, provided herein is an ATDC comprising a Glucagon-like peptide-1 receptor (GLP1R)-targeting antibody or an antigen-binding fragment thereof and a linker-payload having the structure disclosed as SEQ ID NO: 507:

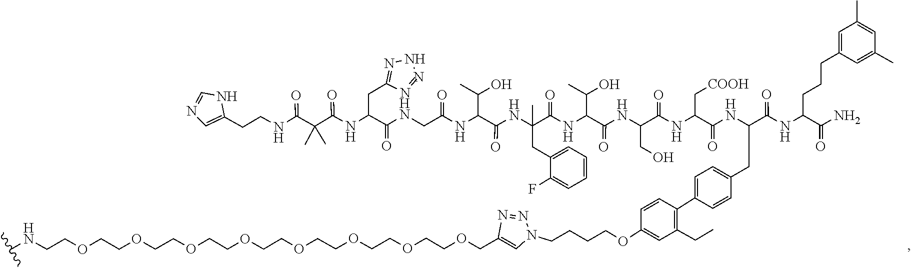

wherein

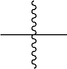

is the point of attachment of the linker-payload to the antibody or the antigen-binding fragment thereof.

In one embodiments, the linker-payload has the structure disclosed as SEQ ID NO: 507:

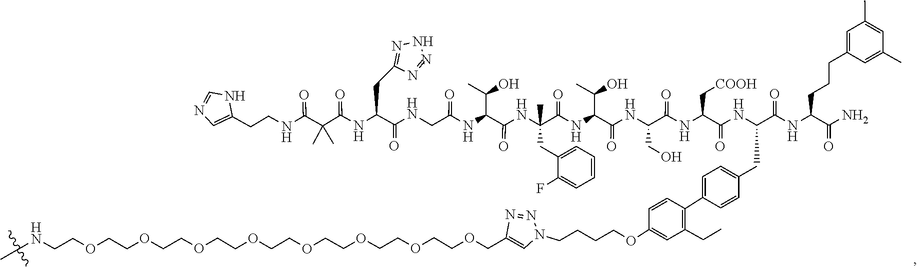

wherein

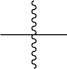

is the point of attachment of the linker-payload to the antibody or the antigen-binding fragment thereof.

### Polynucleotides and Methods of Making

An isolated polynucleotide encoding any of the immunoglobulin chains or portions thereof of antibodies or antigen-binding fragments thereof that bind specifically to GLP1R of the present invention (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) forms part of the present invention as does a vector comprising the polynucleotide and/or a host cell (e.g., Chinese hamster ovary (CHO) cell) comprising the polynucleotide, vector, antibody, antigen-binding fragment and/or a polypeptide set forth herein. Such host cells also form part of the present invention.

Optionally, the polynucleotide is operably linked to a promoter or other expression control sequence. In an embodiment of the invention, a polynucleotide of the present invention is fused to a secretion signal sequence. Polypeptides encoded by such polynucleotides are also within the scope of the present invention.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the invention. Promoters which may be used to control gene expression include, but are not limited to, cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster et al., (1982) Nature 296:39-42); prokaryotic expression vectors such as the beta-lactamase promoter (VIIIa-Komaroff et al., (1978) Proc. Natl. Acad. Sci. USA 75:3727-3731), or the tac promoter (DeBoer et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present invention includes polynucleotides which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (e.g., any of SEQ ID NOs: 14-16); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In an embodiment of the invention, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides relative to any of SEQ ID NOs: 25; 27; 29; 31; 33; 35; 39; 41; 43; 45; 47; 49; 51; 53; 55; 59; 61; 63; 65; 67; 69; 71; 73; 75; 79; 81; 415; 417; 83; 85; 87; 89; 91; 93; 95; 99; 101; 103; 105; 107; 109; 111; 113; 115; 119; 121; 123; 125; 127; 129; 131; 133; 135; 139; 141; 143; 145; 147; 149; 151; 153; 155; 159; 161; 163; 165; 167; 169; 171; 173; 175; 179; 181; 183; 186; 188; 190; 192; 194; 196; 200; 202; 204; 206; 208; 210; 212; 214; 216; 220; 222; 224; 226; 228; 230; 232; 234; 236; 240; 242; 244; 246; 248; 250; 252; 254; 256; 260; 262; 264; 266; 268; 270; 272; 274; 276; 278; 280; 282; 284; 288; 290; 292; 294; 296; 298; 300; 302; 304; 308; 310; 312; 314; 316; 318; 320; 322; 324; 328; 330; 332; 334; 336; 338; 340; 342; 344; 348; 350; 352; 354; 356; 358; 360; 362; 364; 368; 370; 372; 374; 376; 378; 380; 382; 384; 388; 390; 392; 394; 396; 398; 400; 402; 404; 408; 410; or 412 or GGTGCATCC, GCTGCATCC or AAGATTTCT. Such mutations may, in an embodiment of the invention, be missense or nonsense mutations. In an embodiment of the invention, such a variant polynucleotide encodes antibody or antigen-binding fragment immunoglobulin chains that form an antibody or fragment which retains specific binding to GLP1R.

Eukaryotic and prokaryotic host cells, including mammalian cells, may be used as hosts for expression of an antibody or antigen-binding fragment thereof that binds specifically to GLP1R of the present invention. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present invention includes an isolated host cell (e.g., a CHO cell or any type of host cell set forth above) comprising an antibody or fragment, such as REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; and/or REGN9280, and/or a polynucleotide encoding one or more immunoglobulin chains thereof. The present invention includes an isolated host cell (e.g., a CHO cell or any type of host cell set forth above) comprising one or more of such immunoglobulin chains and/or a polynucleotide encoding such chains (e.g., as discussed herein).

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

The present invention includes recombinant methods for making an antibody or antigen-binding fragment thereof that binds specifically to GLP1R or immunoglobulin chain thereof of the present invention (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) comprising
(i) introducing, into a host cell (e.g., CHO or Pichia or Pichia pastoris), one or more polynucleotides (e.g., including the nucleotide sequence in any one or more of SEQ ID NOs: 25; 27; 29; 31; 33; 35; 39; 41; 43; 45; 47; 49; 51; 53; 55; 59; 61; 63; 65; 67; 69; 71; 73; 75; 79; 81; 415; 417; 83; 85; 87; 89; 91; 93; 95; 99; 101; 103; 105; 107; 109; 111; 113; 115; 119; 121; 123; 125; 127; 129; 131; 133; 135; 139; 141; 143; 145; 147; 149; 151; 153; 155; 159; 161; 163; 165; 167; 169; 171; 173; 175; 179; 181; 183; 186; 188; 190; 192; 194; 196; 200; 202; 204; 206; 208; 210; 212; 214; 216; 220; 222; 224; 226; 228; 230; 232; 234; 236; 240; 242; 244; 246; 248; 250; 252; 254; 256; 260; 262; 264; 266; 268; 270; 272; 274; 276; 278; 280; 282; 284; 288; 290; 292; 294; 296; 298; 300; 302; 304; 308; 310; 312; 314; 316; 318; 320; 322; 324; 328; 330; 332; 334; 336; 338; 340; 342; 344; 348; 350; 352; 354; 356; 358; 360; 362; 364; 368; 370; 372; 374; 376; 378; 380; 382; 384; 388; 390; 392; 394; 396; 398; 400; 402; 404; 408; 410; or 412; or a variant thereof; or GGTGCATCC, GCTGCATCC or AAGATTTCT) encoding one or more of the immunoglobulin chains of the present invention (e.g., heavy and light chain immunoglobulin), for example, wherein the polynucleotide is in a vector; and/or integrates into the host cell chromosome and/or is operably linked to a promoter;
(ii) culturing the host cell under conditions favorable to expression of the polynucleotide and,
(iii) optionally, isolating the antibody or antigen-binding fragment thereof or immunoglobulin chain thereof from the host cell and/or medium in which the host cell is grown.

When making antibodies and antigen-binding fragments thereof that bind specifically to GLP1R that includes two or more polypeptide chains, co-expression of the chains in a single host cell leads to association of the chains, e.g., in the cell or on the cell surface or outside the cell if such chains are secreted, so as to form the antibody or fragment. The present invention also includes antibodies and antigen-binding fragments thereof that bind specifically to GLP1R (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) which are the product of the production methods set forth herein, and, optionally, the purification methods set forth herein.

### Antibody Conjugation

Techniques and linkers for conjugating to residues of an antibody or antigen binding fragment thereof are known in the art. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US2012/0585592), cysteine (see, e.g., US2007/0258987; WO2013/055993; WO2013/055990; WO2013/053873; WO2013/053872; WO2011/130598; US2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO2013/068874, and WO2012/166559), and acidic amino acids (see, e.g., WO2012/05982). Lysine conjugation can also proceed through NHS (N-hydroxy succinimide). Linkers can also be conjugated to cysteine residues, including cysteine residues of a cleaved interchain disulfide bond, by forming a carbon bridge between thiols (see, e.g., U.S. Pat. Nos. 9,951,141and 9,950,076). Linkers can also be conjugated to an antigen-binding protein via attachment to carbohydrates (see, e.g., US 2008/0305497, WO2014/065661, and Ryan et al., Food & Agriculture Immunol., 2001, 13:127-130) and disulfide linkers (see, e.g., WO2013/085925, WO2010/010324, WO2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36 (Suppl 1): 100). In specific embodiments discussed in more detail below, Site specific conjugation techniques, include glutamine conjugation via transglutaminase (see e.g., Schibli, Angew Chemie Inter Ed. 2010, 49,9995).

Payloads according to the disclosure linked through lysine and/or cysteine, e.g., via a maleimide or amide conjugation, are included within the scope of the present disclosure.

In some embodiments, the protein-drug conjugates of the present disclosure are produced according to a two-step process, where Step 1 is lysine-based linker conjugation, e.g., with an NHS-ester linker, and Step 2 is a payload conjugation reaction (e.g., a 1,3-cycloaddition reaction).

In some embodiments, the protein-drug conjugates of the present disclosure are produced according to a two-step process, where Step 1 is cysteine-based linker conjugation, e.g., with a maleimide linker, and Step 2 is a payload conjugation reaction (e.g., a 1,3-cycloaddition reaction).

In some embodiments, the protein-drug conjugates of the present disclosure are produced according to a two-step process, where Step 1 is transglutaminase-mediated site specific conjugation and Step 2 is a payload conjugation reaction (e.g., a 1,3-cycloaddition reaction).

### Step 1: Transglutaminase Mediated Site Specific Conjugation

In some embodiments, proteins (e.g., antibodies) may be modified in accordance with known methods to provide glutaminyl modified proteins. Techniques for conjugating antibodies and primary amine compounds are known in the art. Site specific conjugation techniques are employed herein to direct conjugation to glutamine using glutamine conjugation via transglutaminase (see e.g., Schibli, Angew Chemie Inter Ed. 2010, 49, 9995).

Primary amine-comprising compounds (e.g., linkers La) of the present disclosure can be conjugated to one or more glutamine residues of a binding agent (e.g., a protein, e.g., an antibody, e.g., an anti-GLP1R antibody) via transglutaminase-based chemo-enzymatic conjugation (see, e.g., Dennler et al., Protein Conjugate Chem. 2014, 25, 569-578, and WO 2017/147542). For example, in the presence of transglutaminase, one or more glutamine residues of an antibody can be coupled to a primary amine linker compound. Briefly, in some embodiments, a binding agent having a glutamine residue (e.g., a Gln295, i.e. Q295 residue) is treated with a primary amine-containing linker La in the presence of the enzyme transglutaminase. In certain embodiments, the binding agent is aglycosylated. In certain embodiments, the binding agent is deglycosylated.

In certain embodiments, the binding agent (e.g., a protein, e.g., an antibody) comprises at least one glutamine residue in at least one polypeptide chain sequence. In certain embodiments, the binding agent comprises two heavy chain polypeptides, each with one Gln295 residue. In further embodiments, the binding agent comprises one or more glutamine residues at a site other than a heavy chain 295.

In some embodiments, a binding agent, such as an antibody, can be prepared by site-directed mutagenesis to insert a glutamine residue at a site without resulting in disabled antibody function or binding. For example, included herein are antibodies bearing Asn297Gln (N297Q) mutation(s) as described herein. In some embodiments, an antibody having a Gln295 residue and/or an N297Q mutation contains one or more additional naturally occurring glutamine residues in their variable regions, which can be accessible to transglutaminase and therefore capable of conjugation to a linker or a linker-payload. An exemplary naturally occurring glutamine residue can be found, e.g., at Q55 of the light chain. In such instances, the binding agent, e.g., antibody, conjugated via transglutaminase can have a higher than expected LAR value (e.g., a LAR higher than 4). Any such antibodies can be isolated from natural or artificial sources.

### Step 2: Payload Conjugation Reaction

In certain embodiments, linkers La according to the present disclosure comprise at least one first reactive group capable of further reaction after transglutamination. In these embodiments, the glutaminyl-modified protein (e.g., antibody) is capable of further reaction with a reactive payload compound P or a reactive linker-payload compound (e.g., Lp-P as disclosed herein), to form a protein-payload conjugate. More specifically, the reactive linker-payload compound Lp-P may comprise a second reactive group that is capable of reacting with the first reactive group of the linker La. In certain embodiments, a first or second reactive group according to the present disclosure comprises a moiety that is capable of undergoing a 1,3-cycloaddition reaction. In certain embodiments, the reactive group is an azide. In certain embodiments, the reactive group comprises an alkyne (e.g., a terminal alkyne, or an internal strained alkyne). In certain embodiments of the present disclosure the reactive group is compatible with the binding agent and transglutamination reaction conditions.

In certain embodiments of the disclosure, linker La molecules comprise a first reactive group. In certain embodiments of the disclosure, linker La molecules comprise more than one reactive group.

In certain embodiments, the reactive linker-payload Lp-P comprises one payload molecule (n=1). In certain other embodiments, the reactive linker-payload Lp-P comprises two or more payload molecules (n≥2).

In certain embodiments of the disclosure, the drug-antibody ratio or DAR is from about 1 to about 30, or from about 1 to about 24, or from about 1 to about 20, or from about 1 to about 16, or from about 1 to about 12, or from about 1 to about 10, or from about 1 to about 8, or about 1, 2, 3, 4, 5, 6, 7, or 8 payload molecules per antibody. In some embodiments, the DAR is from 1 to 30. In some embodiments, the DAR is from 1 to 16. In some embodiments, the DAR is from 1 to 8. In some embodiments, the DAR is from 1 to 6. In certain embodiments, the DAR is from 2 to 4. In some cases, the DAR is from 2 to 3. In certain cases, the DAR is from 0.5 to 3.5. In some embodiments, the DAR is about 1, or about 1.5, or about 2, or about 2.5, or about 3, or about 3.5. In some embodiments, the DAR is 2. In some embodiments, the DAR is 4. In some embodiments, the DAR is 8.

In one aspect, the present disclosure provides a method of producing the ATDC having a structure of Formula (A):

BA-(L-P)ₘ (A),

the method comprising the steps of:
a) contacting, in the presence of a transglutaminase, the BA comprising at least m glutamine residues Gln with at least m equivalents of compound L-P, and
b) isolating the produced ATDC of Formula (A)
   wherein BA is the anti-GLP1R antibody or antigen-binding fragment thereof:
(i) comprising a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413, or a variant thereof;
(ii) which is an antibody or antigen-binding fragment thereof that competes for binding to GLP1R with said antibody or fragment of (i); and/or
(iii) which is an antibody or antigen-binding fragment thereof that binds to the same epitope of GLP1R as said antibody or fragment of (i).

In one aspect, the present disclosure provides a method of producing the ATDC having a structure of Formula (A):

BA-(L-P)ₘ (A),
wherein the linker L has has the structure of formula (L′):

—La—Y-Lp- (L′),

wherein La is a first linker covalently attached to the BA;
Y is a group comprising a triazole, and
Lp is a second linker covalently attached to the P,
   the method comprising the steps of:
a) contacting, in the presence of a transglutaminase, the BA comprising at least m glutamine residues Gln with the first linker La comprising an azide or an alkyne moiety;
b) contacting the product of step a) with at least m equivalents of compound Lp-P, wherein the second linker Lp comprises an azide or an alkyne moiety, wherein La and Lp are capable of reacting to produce a triazole, and
c) isolating the produced ATDC of Formula (A);
   wherein BA is the anti-GLP1R antibody or antigen-binding fragment thereof:
(i) comprising a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413, or a variant thereof;
(ii) which is an antibody or antigen-binding fragment thereof that competes for binding to GLP1R with said antibody or fragment of (i); and/or
(iii) which is an antibody or antigen-binding fragment thereof that binds to the same epitope of GLP1R as said antibody or fragment of (i).

In one aspect, the present disclosure provides a method of producing a ATDC having a structure according to Formula (I):

BA-L-P (I),

the method comprising the steps of:
a) contacting, in the presence of a transglutaminase, the BA comprising at least one glutamine residue Gln with a compound L-P, and
b) isolating the produced ATDC of Formula (I),
   wherein BA is the anti-GLP1R antibody or antigen-binding fragment thereof:
(i) comprising a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413, or a variant thereof;
(ii) which is an antibody or antigen-binding fragment thereof that competes for binding to GLP1R with said antibody or fragment of (i); and/or
(iii) which is an antibody or antigen-binding fragment thereof that binds to the same epitope of GLP1R as said antibody or fragment of (i); L is a non-cleavable linker; P is a payload having the structure selected from the group consisting of (SEQ ID NOS 451-452, respectively, in order of appearance):

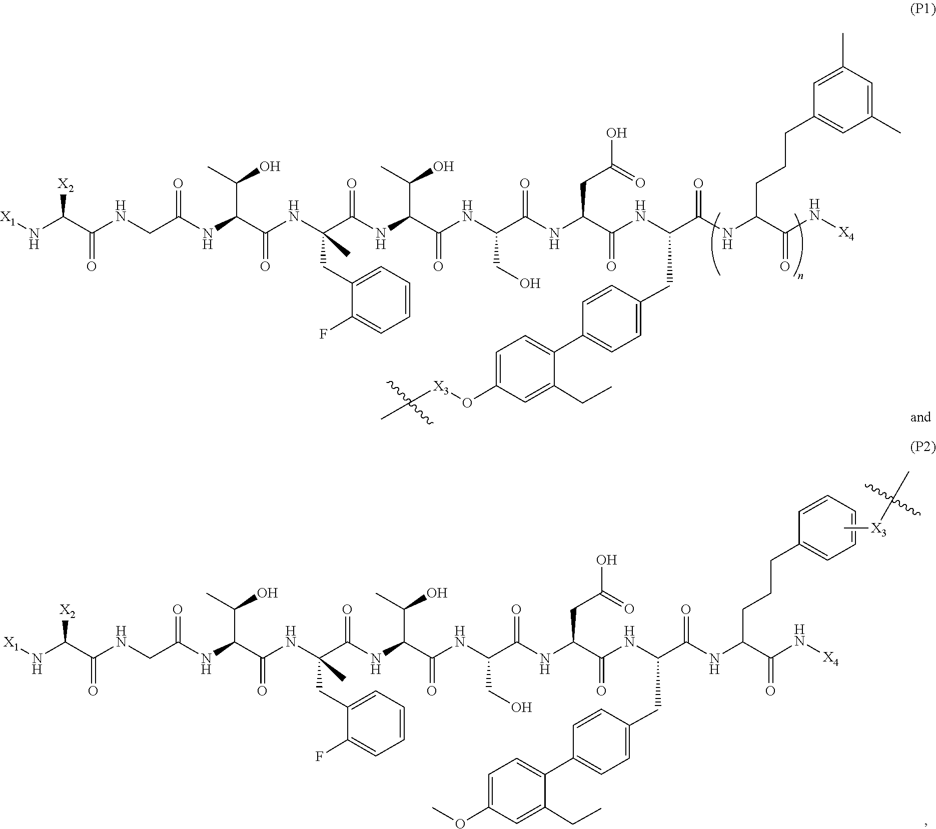

wherein

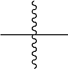

is the point of attachment of the payload to L;
X₁ is selected from H;

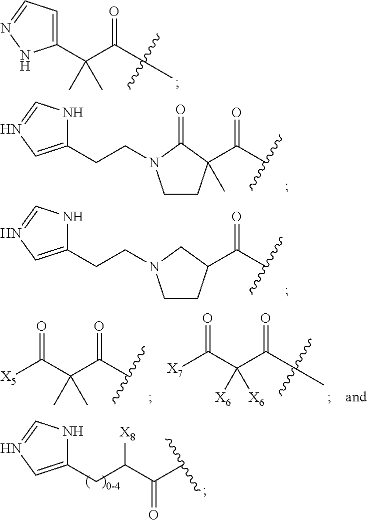

X₂ is selected from

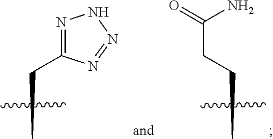

X₃ is selected from —(CH₂)₂₋₆—NH— and —(CH₂)₂₋₆-Tr-, where Tr is a triazole moiety;
n is 0 or 1;
X₄ is selected from H and phenyl;
X₅ is selected from —OH, —NH₂, —NH—OH, and

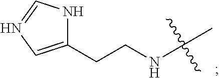 [SMILES: *NCCc1cnc[nH]1]

X₆ is independently at each occurrence selected from H, —OH, —CH₃, and —CH₂OH;
X₇ is selected from H,

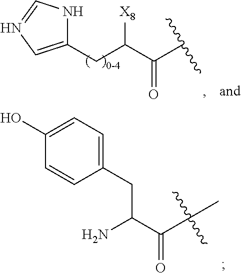

X₈ is selected from H, —OH, —NH₂, and

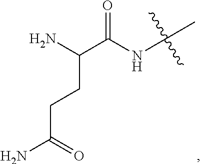 [SMILES: *NC(=O)C(N)CCC(N)=O]

or a pharmaceutically acceptable salt thereof.

In another aspect, the present disclosure provides a method of producing a ATDC having a structure according to Formula (I):

BA-L-P (I),
wherein the linker L has has the structure of formula (L′):

—La—Y-Lp- (L′),

wherein La is a first linker covalently attached to the BA;
Y is a group comprising a triazole, a Diels-Alder adduct, or a thio-maleimide adduct, and
Lp is a second linker covalently attached to the P,
   the method comprising the steps of:
a) contacting, in the presence of a transglutaminase, the BA comprising at least one glutamine residue Gln with the first linker La comprising an azide or an alkyne moiety;
b) contacting the product of step a) with a compound Lp-P, wherein the second linker Lp comprises an azide or an alkyne moiety, wherein La and Lp are capable of reacting to produce a triazole, and
c) isolating the produced ATDC of Formula (I),
   wherein BA, L′, and P are as defined above.

In one embodiment of the invention, Y is a group comprising a triazole.

In one embodiment of the invention, the glutamine residue Gln is naturally present in a CH2 or CH3 domain of the BA. In another embodiment of the invention, the glutamine residue Gln is introduced to the BA by modifying one or more amino acids. In one embodiment, the Gln is Q295 or N297Q.

In one embodiment of the invention, the transglutaminase is microbial transglutaminase (MTG). In one embodiment, the transglutaminase is bacterial transglutaminase (BTG).

In one embodiment of the invention, the compound L-P for use in any of the above methods of producing the ATDC of Formula (I) has a structure selected from the group consisting of: (SEQ ID NOS 514, 514, 514, 514, 514-519, 519, 519-532, 515-516 and 534-536 and 538, respectively, in order of appearance)

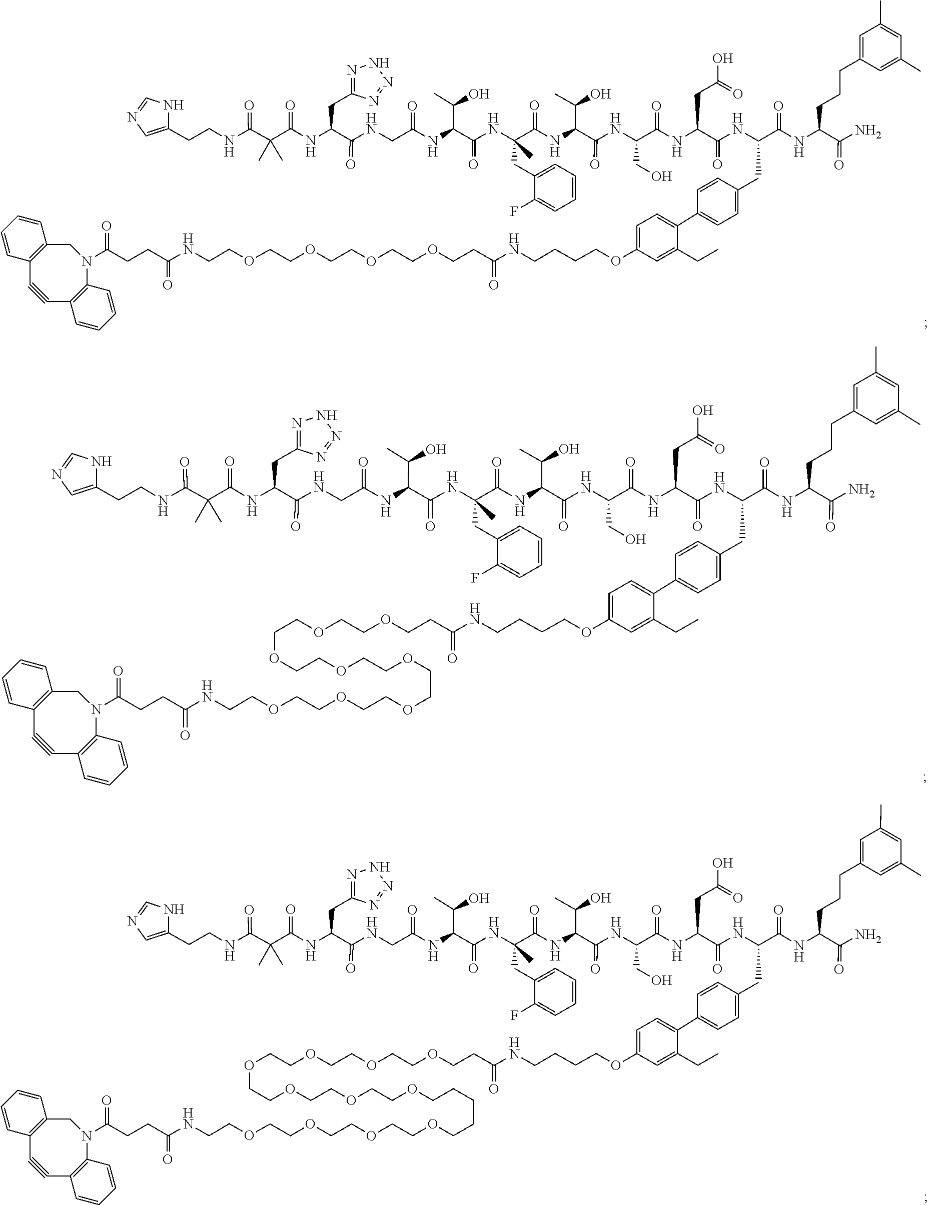

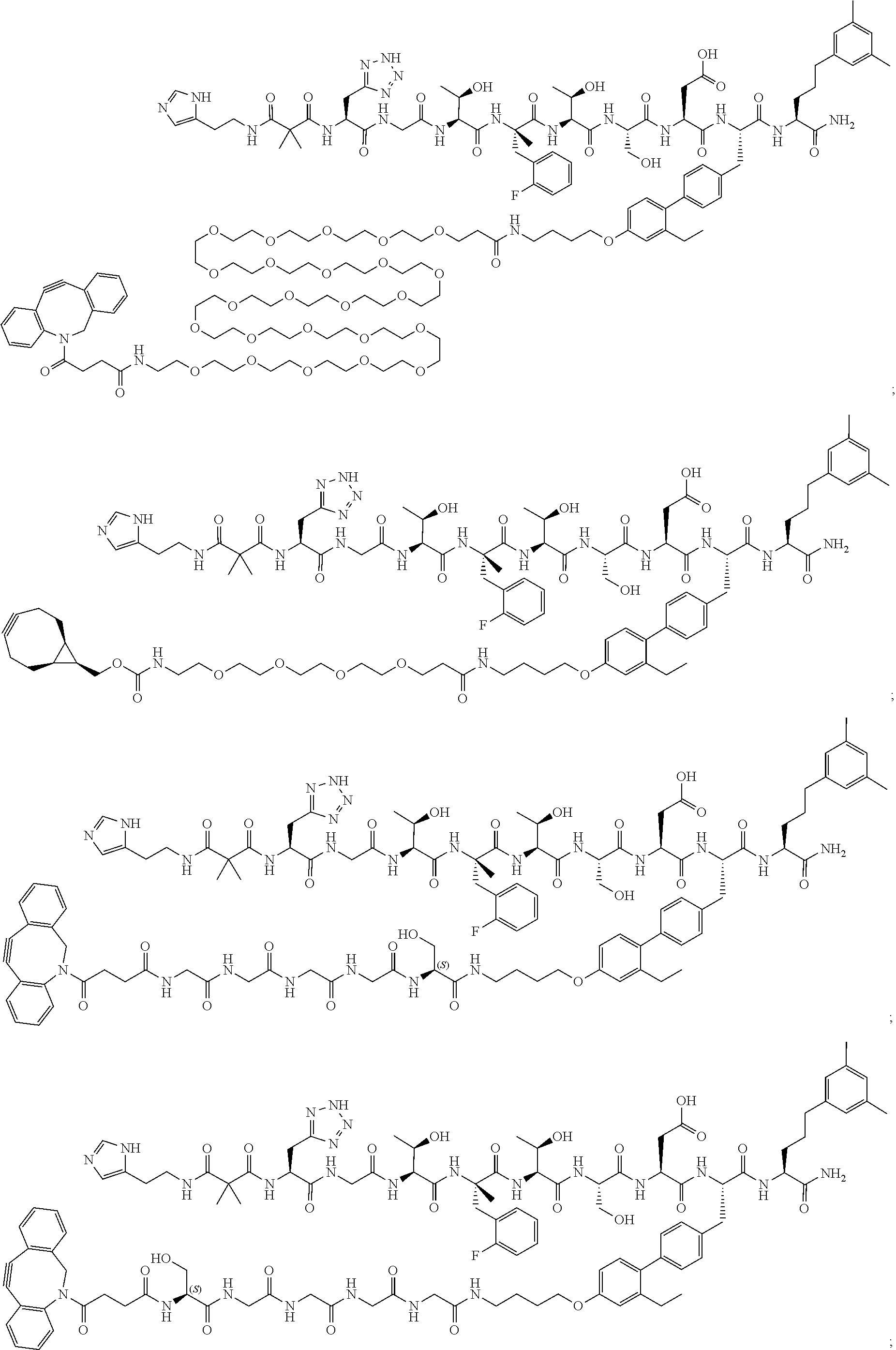

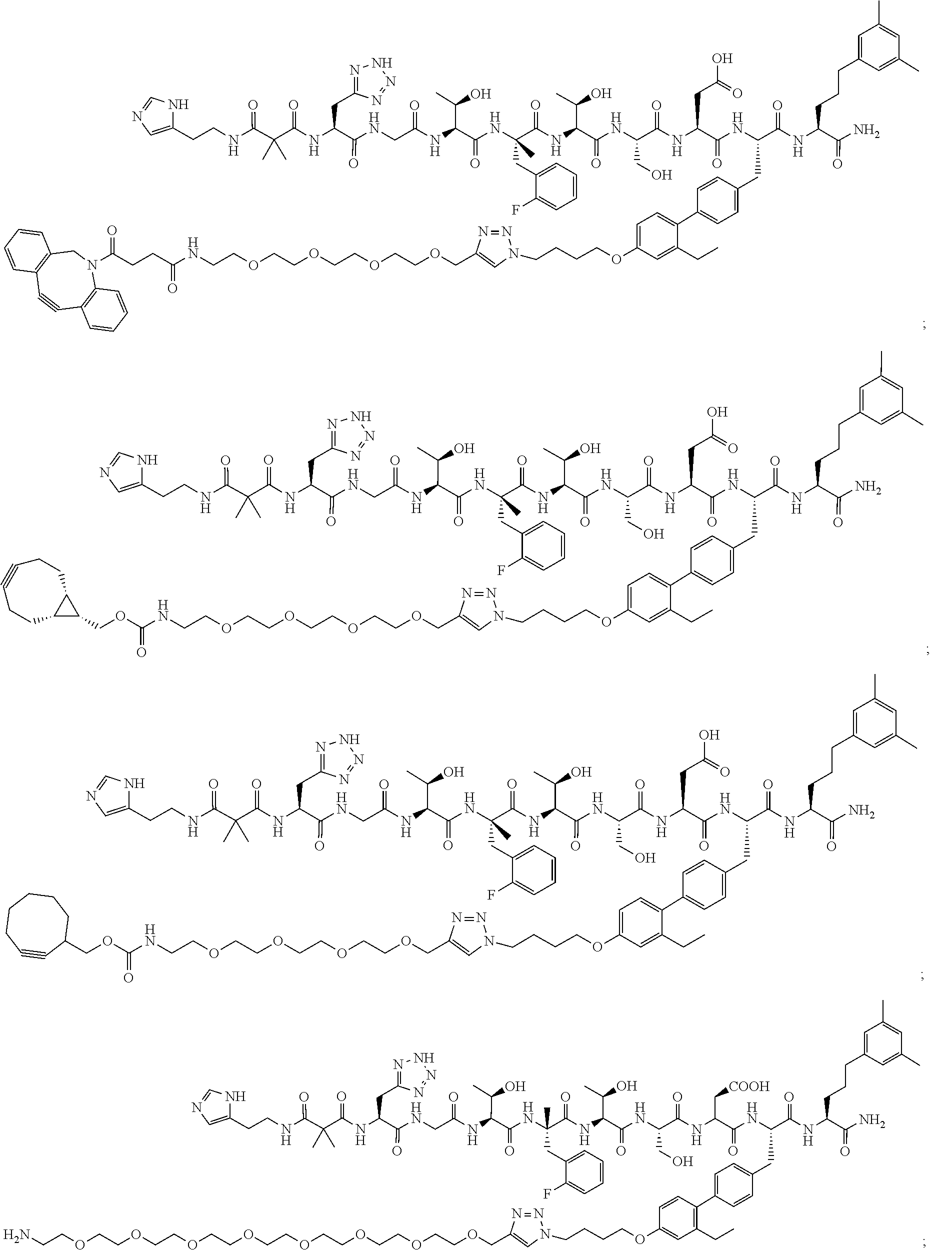

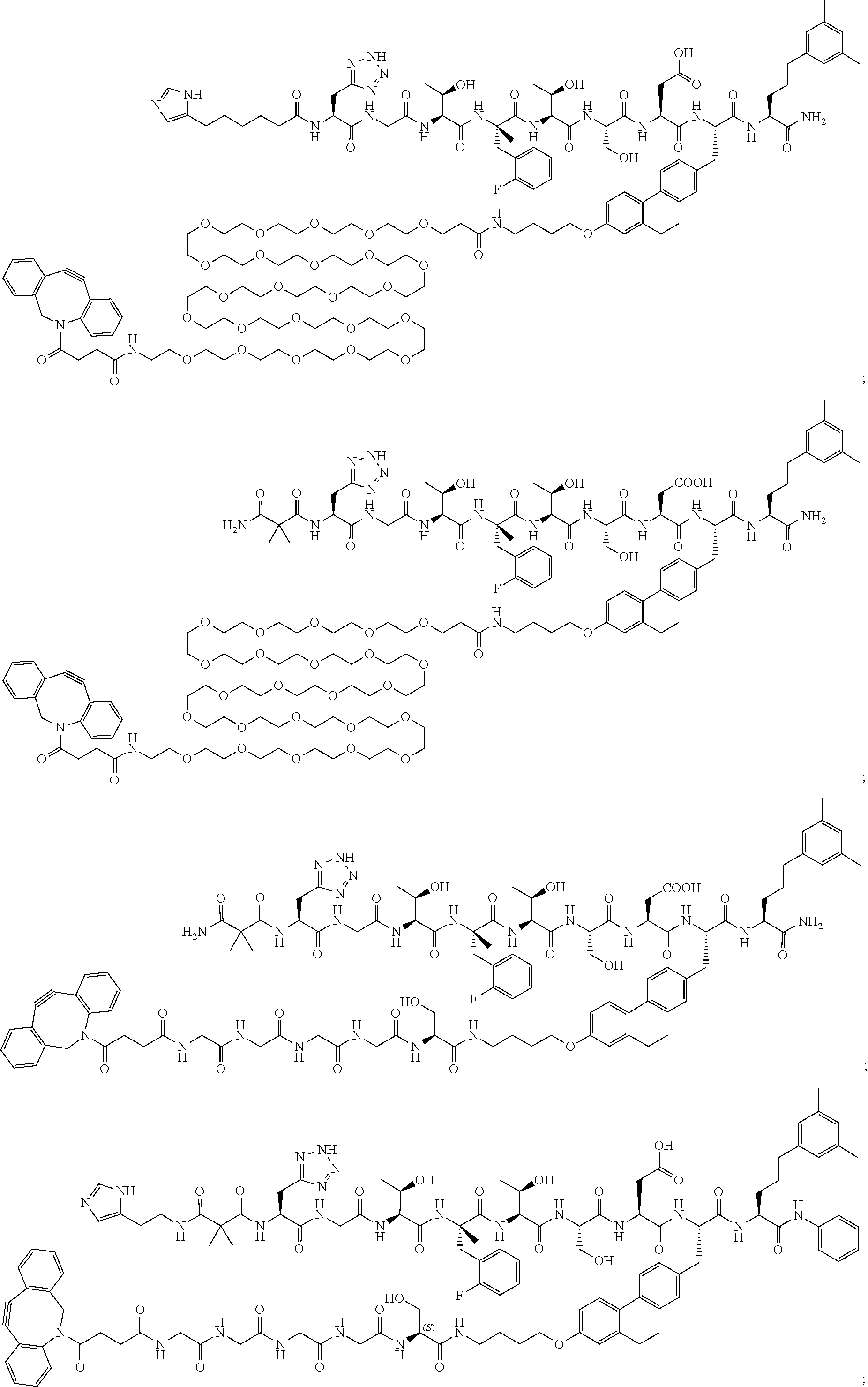

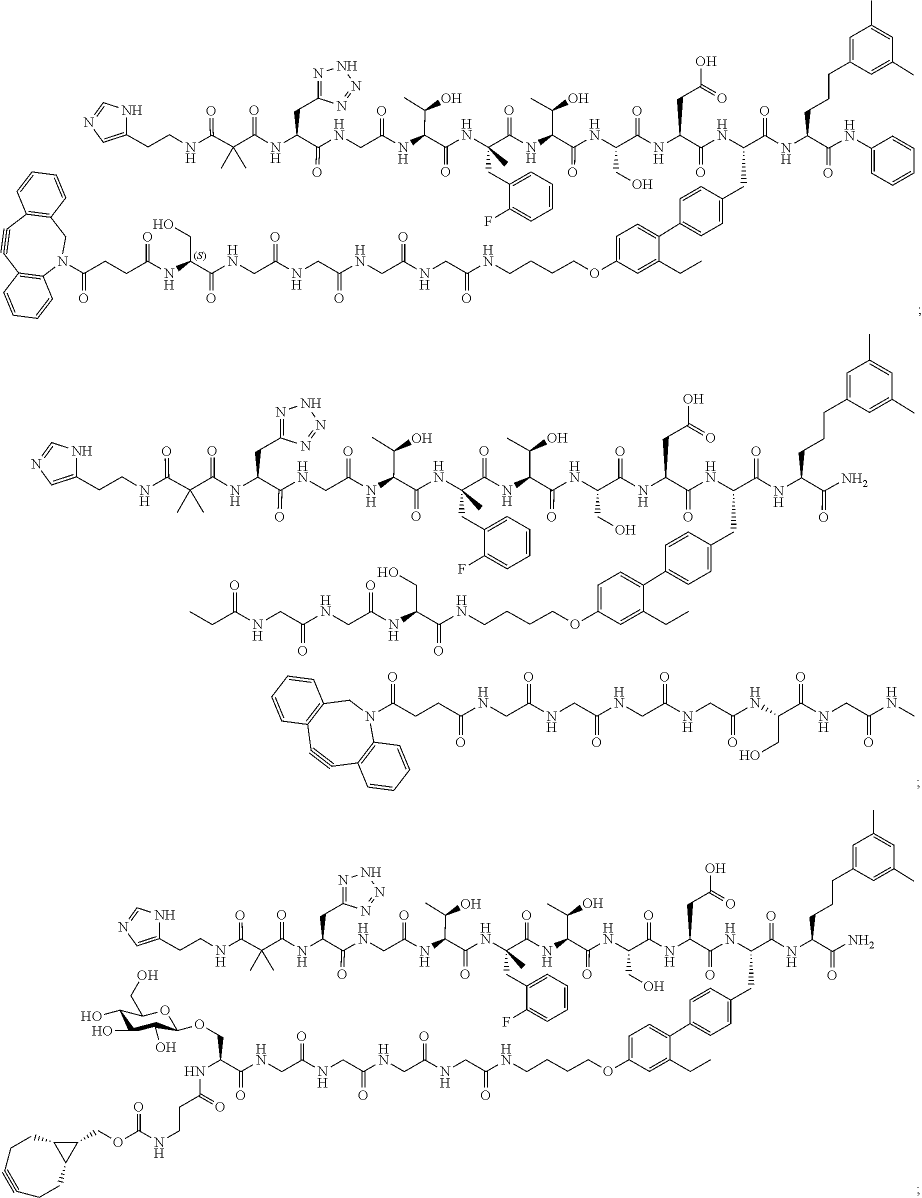

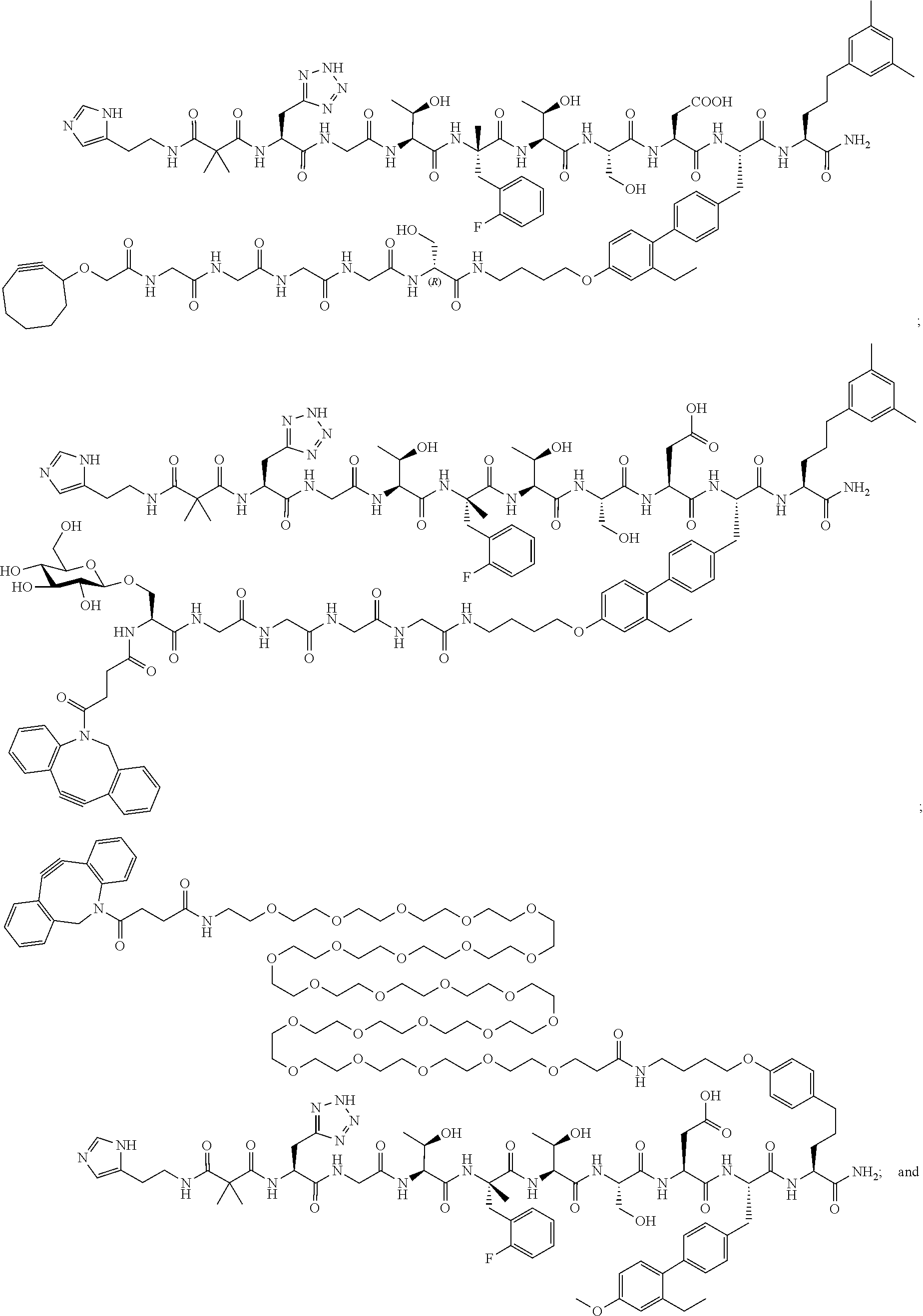

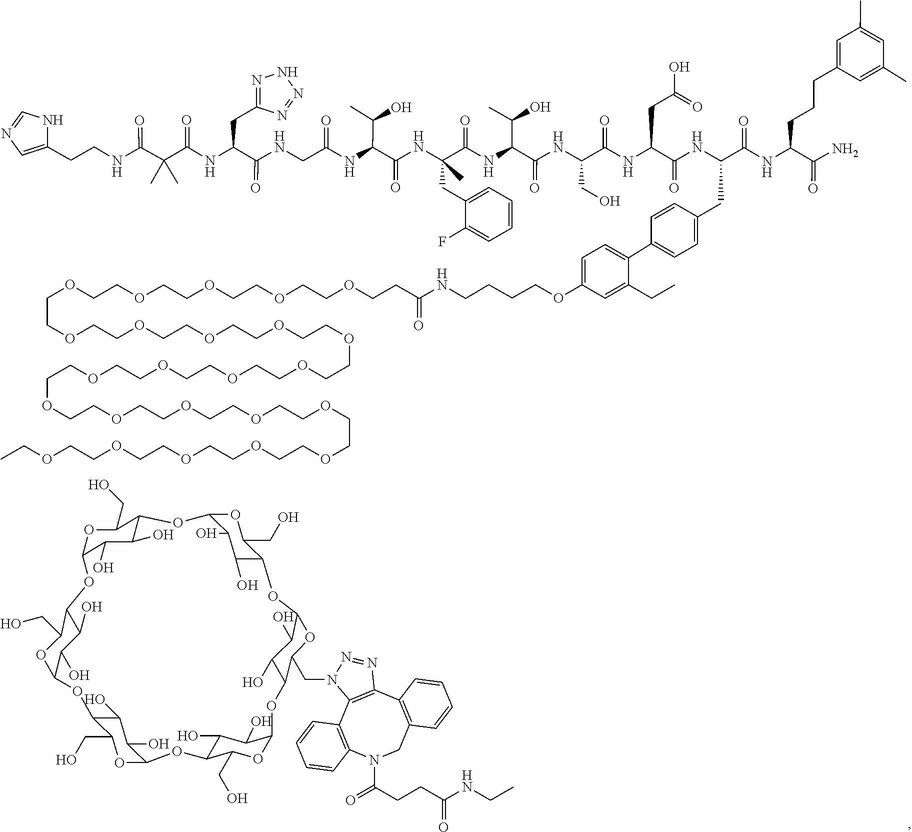

or a pharmaceutically acceptable salt thereof.

### Therapeutic Formulations, Administration and Uses

The present invention provides compositions that include the antibodies and antigen-binding fragments that bind specifically to GLP1R set forth herein (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) and one or more ingredients; as well as methods of use thereof and methods of making such compositions.

To prepare pharmaceutical compositions of antibodies and antigen-binding fragments that bind specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32), the antibody or fragment is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, N Y; Lieberman, et al. (eds.) (1990) Pharmaceutical Dosage Forms: Tablets, Marcel Dekker, N Y; Lieberman, et al. (eds.) (1990) Pharmaceutical Dosage Forms: Disperse Systems, Marcel Dekker, N Y; Weiner and Kotkoskie (2000) Excipient Toxicity and Safety, Marcel Dekker, Inc., New York, N.Y. See also Powell et al. “Compendium of excipients for parenteral formulations” PDA (1998) J Pharm Sci Technol 52:238-311. In an embodiment of the invention, the pharmaceutical composition is sterile. Such pharmaceutical compositions are part of the present invention.

The present invention provides pharmaceutical compositions comprising the antibody or antigen-binding fragment or the antibody-tethered drug conjugate described above, wherein at least about 80% of the antibody-tethered drug conjugate does not comprise a C-terminal lysine in any of the heavy chains. In some embodiments, the pharmaceutical composition comprises at least about 90% of the antibody or antigen-binding fragment or antibody-tethered drug conjugate that does not have a C-terminal lysine in any of the heavy chains. In some embodiments, the pharmaceutical composition comprises at least about 95% of the antibody or antigen-binding fragment or antibody-tethered drug conjugate that does not have a C-terminal lysine in any of the heavy chains. In some embodiments, the pharmaceutical composition comprises at least about 99% of the antibody or antigen-binding fragment or antibody-tethered drug conjugate that does not have a C-terminal lysine in any of the heavy chains. In some embodiments, the pharmaceutical composition comprises about 80%-90%, 80%-95%, 80%-99%, 80%-100%, 85%-90%, 85%-95%, 85%-99%, 85%-100%, 90%-95%, 90%-99%, 90%-100%, 95%-99%, or 95%-100% of the antibody or antigen-binding fragment or antibody-tethered drug conjugate that does not have a C-terminal lysine in any of the heavy chains. In some embodiments, the pharmaceutical composition comprises about 80% of the antibody or antigen-binding fragment or antibody-tethered drug conjugate that does not have a C-terminal lysine in any of the heavy chains. In some embodiments, the pharmaceutical composition comprises about 90% of the antibody or antigen-binding fragment or antibody-tethered drug conjugate that does not have a C-terminal lysine in any of the heavy chains. In some embodiments, the pharmaceutical composition comprises about 95% of the antibody or antigen-binding fragment or antibody-tethered drug conjugate that does not have a C-terminal lysine in any of the heavy chains. In some embodiments, the pharmaceutical composition comprises about 99% of the antibody or antigen-binding fragment or antibody-tethered drug conjugate that does not have a C-terminal lysine in any of the heavy chains. In some embodiments, the pharmaceutical composition comprises about 100% of the antibody or antigen-binding fragment or antibody-tethered drug conjugate that does not have a C-terminal lysine in any of the heavy chains.

The present invention also provides pharmaceutical compositions comprising the antibody or antigen-binding fragment or the antibody-tethered drug conjugate described above, wherein the antibody or antigen-binding fragment or antibody-tethered drug conjugate comprises at least one heavy chain immunoglobulin that comprises the amino acid sequence SEQ ID NO: 414, or 416, or a variant thereof.

The present invention further provides pharmaceutical compositions comprising the antibody or antigen-binding fragment or the antibody-tethered drug conjugate described above, wherein less than about 20% of the antibody or antigen-binding fragment or antibody-tethered drug conjugate comprises a C-terminal lysine in at least one heavy chain. In some embodiments, less than about 10% of the antibody or antigen-binding fragment or antibody-tethered drug conjugate comprises a C-terminal lysine in at least one heavy chain. In some embodiments, less than about 5% of the antibody or antigen-binding fragment or antibody-tethered drug conjugate comprises a C-terminal lysine in at least one heavy chain. In some embodiments, less than about 1% of the antibody or antigen-binding fragment or antibody-tethered drug conjugate comprises a C-terminal lysine in at least one heavy chain. In some embodiments, less than about 1%-20%, 5%-20%, 10%-20%, 15%-20%, 1%-15%, 5%-15%, 10%-15%, 1%-10%, or 5%-10% of the antibody or antigen-binding fragment or antibody-tethered drug conjugate comprises a C-terminal lysine in at least one heavy chain. In some embodiments, about 20% of the antibody or antigen-binding fragment or antibody-tethered drug conjugate comprises a C-terminal lysine in at least one heavy chain. In some embodiments, about 10% of the antibody or antigen-binding fragment or antibody-tethered drug conjugate comprises a C-terminal lysine in at least one heavy chain. In some embodiments, about 5% of the antibody or antigen-binding fragment or antibody-tethered drug conjugate comprises a C-terminal lysine in at least one heavy chain. In some embodiments, about 1% of the antibody or antigen-binding fragment or antibody-tethered drug conjugate comprises a C-terminal lysine in at least one heavy chain. In some embodiments, about 0% of the antibody or antigen-binding fragment or antibody-tethered drug conjugate comprises a C-terminal lysine in at least one heavy chain.

The present invention additionally provides pharmaceutical compositions comprising the antibody or antigen-binding fragment or the antibody-tethered drug conjugate described above, wherein the antibody or antigen-binding fragment or antibody-tethered drug conjugate comprising at least one heavy chain that comprises the amino acid sequence SEQ ID NO: 42; 62; 82; 102; 122; 142; 162; 182; 203; 223; 243; 263; 267; 271; 291; 311; 331; 351; 371; 391; or 411; or a variant thereof. In some embodiments, the antibody or antigen-binding fragment or antibody-tethered drug conjugate comprises at least one heavy chain that comprises the amino acid sequence SEQ ID NO: 82.

Pharmaceutical compositions of the present invention include pharmaceutically acceptable carriers, diluents, excipients and/or stabilizers, such as, for example, water, buffering agents, stabilizing agents, preservatives, isotonifiers, non-ionic detergents, antioxidants and/or other miscellaneous additives.

In one aspect of the invention, the present disclosure provides compositions comprising a population of ATDCs according to the present disclosure having a drug-antibody ratio (DAR) of about 0.5 to about 30.0.

In one embodiment of the invention, the composition has a DAR of about 1.0 to about 2.5.

In one embodiment of the invention, the composition has a DAR of about 2.

In one embodiment of the invention, the composition has a DAR of about 3.0 to about 4.5.

In one embodiment of the invention, the composition has a DAR of about 4.

In one embodiment of the invention, the composition has a DAR of about 6.5 to about 8.5.

In one embodiment of the invention, the composition has a DAR of about 8.

“Treat” or “treating” means to administer an ATDC of the present invention (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32), to a subject, having a GLP1R-associated condition, such that one or more signs and/or symptoms of the GLP1R-associated condition regresses or is eliminated and/or the progression of one or more signs and/or symptoms of the condition is inhibited (e.g., the presence of the condition itself in the subject).

The phrase “therapeutically effective” amount of ATDC refers to an amount effective or sufficient in treating a GLP1R-associated condition. The therapeutically effective amount of ATDC set forth herein (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) administered to a patient may vary depending upon the age and the size of the patient, target disease, conditions, route of administration, and the like. The suitable dose is typically calculated according to body weight or body surface area. When an ATDC of the present disclosure is used for therapeutic purposes in an adult patient, it may be advantageous to intravenously administer the ATDC of the present disclosure normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering an antibody or fragment may be determined empirically; for example, patient progress can be monitored by periodic assessment, and the dose adjusted accordingly.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

The present invention includes a method for administering an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) to a subject (e.g., a subject suffering from a GLP1R-associated condition) comprising introducing the antibody or fragment into the body of the subject (e.g., parenterally or non-parenterally).

In another aspect, the ATDCs that bind specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) disclosed herein are useful, inter alia, for the treatment, prevention and/or amelioration of a disease, disorder or condition in need of such treatment.

In one aspect, the present disclosure provides a method of treating a condition in a subject in need thereof comprising administering to the subject a therapeutically effective amount of an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) according to the disclosure, or a composition comprising any ATDC of the present invention. In an embodiment of the invention, the linker-payload of the ATDC is M3190.

In some embodiments, an ATDC that binds specifically to GLP1R (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload (LP) is LP11, LP30, or LP32) disclosed herein is useful for treating any disease or disorder in which stimulation, activation and/or targeting of GLP1R would be beneficial. In particular, the anti-GLP1R ATDCs of the present disclosure can be used for the treatment, prevention and/or amelioration of any disease or disorder associated with or mediated by GLP1R expression or activity. In an embodiment of the invention, the linker-payload of the ATDC is M3190.

In some embodiments, an ATDC that binds specifically to GLP1R (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) disclosed herein is useful for treating a GLP1R-associated condition. In some embodiments, the GLP1R-associated condition is Type 1 or Type 2 diabetes mellitus. The administered ATDC may cause at least one of the following results: induction of insulin secretion, suppression of glucagon release, reduction of blood sugar, improvement of glycemic control, promotion of islet neogenesis, and delay of gastric emptying or potentiation of glucose resistant islets. In an embodiment of the invention, the linker-payload of the ATDC is M3190.

In some embodiments, the GLP1R-associated condition is a neurodegenerative disorder, a cognitive disorder, memory disorder or learning disorder. The neurodegenerative disorder may be, for example, dementia, senile dementia, mild cognitive impairment, Alzheimer-related dementia, Huntington's chores, tardive dyskinesia, hyperkinesias, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis, glaucoma and Alzheimer's disease.

In some embodiments, the GLP1R-associated condition is a liver disease. The liver disease may be, for example, non-alcoholic fatty liver disease (NAFLD), fatty liver, non-alcoholic steatohepatitis (NASH), and cirrhosis.

In some embodiments, the GLP1R-associated condition is a coronary artery disease. The coronary artery disease may be, for example, cardiomyopathy and myocardial infarction.

In some embodiments, the GLP1R-associated condition is a kidney disease. The kidney disease may be, for example, hypertension, or chronic kidney failure.

In some embodiments, the GLP1R-associated condition is an eating disorder. The eating disorder may be, for example, binge eating.

Without wishing to be bound by theory, an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) disclosed herein may be employed to attenuate the effects of apoptosis-mediated degenerative diseases of the central nervous system such as Alzheimer's Disease, Creutzfeld-Jakob Disease and bovine spongiform encephalopathy, chronic wasting syndrome and other prion mediated apoptotic neural diseases (see, e.g., Perry and Grieg (2004) Current Drug Targets 6:565-571). Administration of the antibody or fragment disclosed herein may also lead to down-modulation of βAPP and thereby ameliorate Aβ mono- or oligomer-mediated pathologies associated with Alzheimer's Disease (see, e.g., Perry et al. (2003) Journal of Neuroscience Research 72: 603-612).

It is also contemplated that an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) disclosed herein may be used to improve learning and memory, for example, by enhancing neuronal plasticity and facilitation of cellular differentiation (see, During et al. (2003) Nature Medicine 9:1173-1179). Further, the ATDCs disclosed herein may also be used to preserve dopamine neurons and motor function in Morbus Parkinson (see, e.g., Greig et al. (2005) Abstract 897.6, Society for Neuroscience, Washington, D.C.). In an embodiment of the invention, the linker-payload of the ATDC is M3190.

In some embodiments of the invention, an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) disclosed herein may also be used to treat a metabolic disorder. The metabolic disorder may be, for example, obesity, dyslipidemia, metabolic syndrome X, and pathologies emanating from islet insufficiency. In an embodiment of the invention, the linker-payload of the ATDC is M3190.

Additional diseases that may be treated by an ATDC that binds specifically to GLP1R (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) of the present disclosure include autoimmune diseases, in particular, those associated with inflammation, including, but not limited to, autoimmune diabetes, adult onset diabetes, morbid obesity, Metabolic Syndrome X and dyslipidemia. For example, the ATDC that binds specifically to GLP1R (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) can be employed as a growth factor for the promotion of islet growth in persons with autoimmune diabetes. The antibody or fragment described herein may also be useful in the treatment of congestive heart failure. In an embodiment of the invention, the linker-payload of the ATDC is M3190.

In one aspect, the present disclosure provides a method of selectively targeting an antigen (e.g., GLP1R) on a surface of a cell with an ATDC. In one embodiment of the invention, the method of selectively targeting an antigen (e.g., GLP1R) on a surface of a cell with a ATDC comprises linking a payload or linker-payload to a targeted antibody (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., wherein the linker-payload is LP11, LP30 or LP32) that binds specifically to GLP1R. In an embodiment of the invention, the linker-payload of the ATDC is M3190. In one embodiment, the payload is as described herein. In one embodiment of the invention, the cell is a mammalian cell. In one embodiment, the cell is a human cell. In one embodiment, the cell is a pancreatic cell or a brain cell. In certain embodiments, the present disclosure provides a method of selectively targeting an antigen such as GLP1R on a surface of a cell with a compound having the structure selected from the group consisting of (SEQ ID NOS 451-452, respectively, in order of appearance):

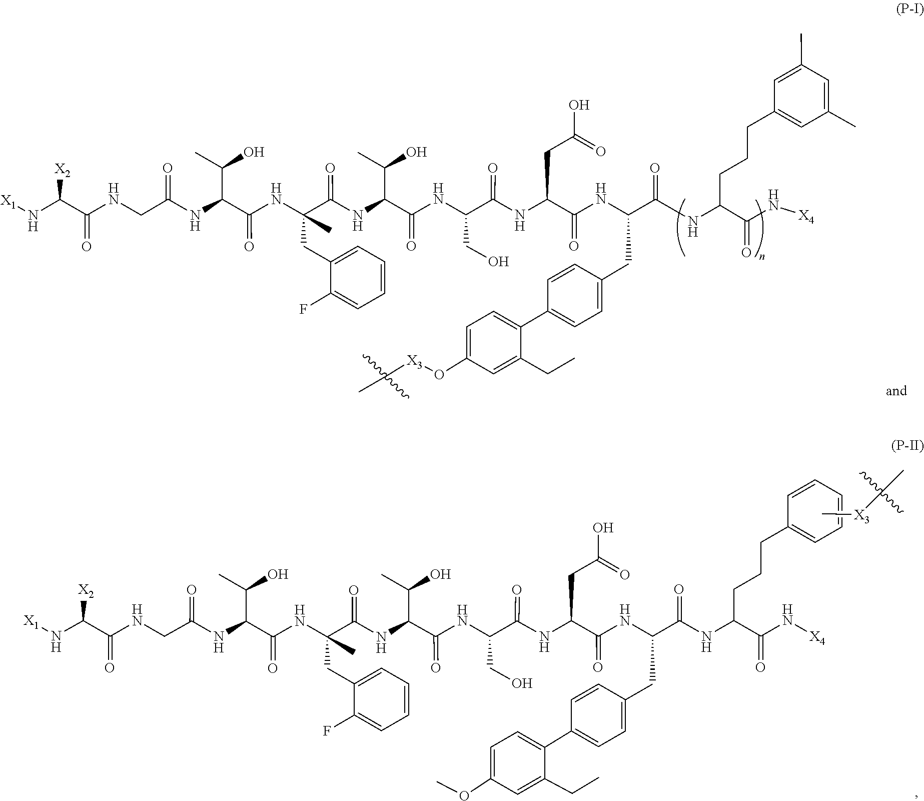

wherein

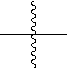

is the point of attachment of the compound to a linker L;
X₁ is selected from H

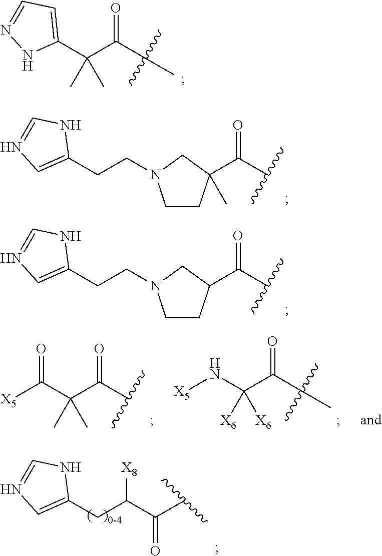

X₂ is selected from

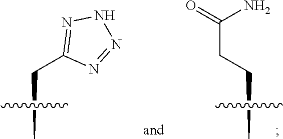

X₃ is selected from —(CH₂)₂₋₆—NH— and —(CH₂)₂₋₆-Tr-, where Tr is a triazole moiety;
n is 0 or 1;
X₄ is selected from H and phenyl;
X₅ is selected from —OH, —NH₂, —NH—OH, and

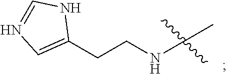 [SMILES: *NCCc1cnc[nH]1]

X₆ is independently at each occurrence selected from H, —OH, —CH₃, and —CH₂OH;
X₇ is selected from H,

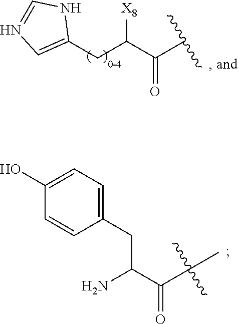

X₈ is selected from H, —OH, —NH₂, and

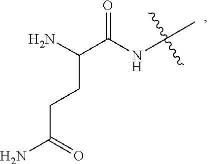 [SMILES: *NC(=O)C(N)CCC(N)=O]

or a pharmaceutically acceptable salt thereof.

In certain embodiments of the invention, the present disclosure also includes the use of an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) of the present disclosure in the manufacture of a medicament for the treatment of a disease or disorder (e.g., cancer) related to or caused by GLP1R-expressing cells. In one aspect, the present disclosure relates to an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) as disclosed herein, for use in medicine. In one aspect of the invention, the present disclosure relates to an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) as disclosed herein, for use in medicine. In an embodiment of the invention, the linker-payload of the ATDC is M3190.

### Combination Therapies and Formulations

The present disclosure provides methods which comprise administering a pharmaceutical composition comprising an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) in association with one or more additional therapeutic agents. Compositions (e.g., co-formulations or kits) comprising an ATDC that binds specifically to GLP1R in association with an additional therapeutic agent also form part of the present invention. In an embodiment of the invention, the linker-payload of the ATDC is M3190.

Exemplary additional therapeutic agents that may be in association with an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) of the present disclosure include, other GLP1R agonists (e.g., an anti-GLP1R antibody or a small molecule agonist of GLP1R or an anti-GLP1R antibody-drug conjugate). Non-limiting examples of GLP1R agonists include exenatide (Byetta, Bydureon), liraglutide (Victoza, Saxenda), lixisenatide (Lyxumia in Europe, Adlyxin in the United States), albiglutide (Tanzeum), dulaglutide (Trulicity), semaglutide (Ozempic), and taspoglutide.

Exemplary additional therapeutic agents may include dual or triple-agonists, including GLP1R/GIPR dual agonists, such as GLP1R/GCGR dual agonists, GLP1R/GIPR/GCGR triple-agonists.

Other agents that may be in association with an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) of the disclosure include those that are useful in the treatment of diabetes (e.g., type II diabetes), obesity, and/or other related metabolic diseases.

In some embodiments, the additional therapeutic agent is an antidiabetic agent. Any suitable antidiabetic agents can be used. Non-limiting examples of antidiabetic agents include insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones or TZDs (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, glucokinase activators, squalene synthase inhibitors, other lipid lowering agents and aspirin. In some such embodiments, the antidiabetic agent is an oral antidiabetic agents (OAA) such as metformin, acarbose, or TZDs. In some such embodiments, the antidiabetic agent is metformin.

In some embodiments of the invention, the ATDC and one or more antidiabetic agents may be formulated into the same dosage form, such as a solution or suspension for parenteral administration.

The present disclosure includes pharmaceutical compositions in which ATDCs of the present disclosure are co-formulated with one or more of the additional therapeutically active component(s) as described elsewhere herein.

The term “in association with” indicates that components, an ATDC of the present invention, along with another agent, such as insulin, can be formulated into a single composition, e.g., for simultaneous delivery, or formulated separately into two or more compositions (e.g., a kit including each component). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route.

### Administration Regimens

According to certain embodiments of the present invention, multiple doses of with an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) may be administered to a subject over a defined time course. The methods according to this aspect of the disclosure comprise sequentially administering to a subject multiple doses of ATDC that binds specifically to GLP1R of the disclosure. As used herein, “sequentially administering” means that each dose of ATDC that binds specifically to GLP1R is administered to the subject at a different point in time, e.g., on different days separated by a predetermined interval (e.g., hours, days, weeks or months). The present disclosure includes methods which comprise sequentially administering to the patient a single initial dose of ATDC that binds specifically to GLP1R, followed by one or more secondary doses of the ATDC that binds specifically to GLP1R, and optionally followed by one or more tertiary doses of the ATDC that binds specifically to GLP1R.

The terms “initial dose,” “secondary doses,” and “tertiary doses,” refer to the temporal sequence of administration of the ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the ATDC that binds specifically to GLP1R, but generally may differ from one another in terms of frequency of administration. In certain embodiments, however, the amount of the ATDC that binds specifically to GLP1R contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1%, 2, 2%, 3, 3%, 4, 4%, 5, 5%, 6, 6%, 7, 7%, 8, 8%, 9, 9%, 10, 10%, 11, 11%, 12, 12%, 13, 13%, 14, 14%, 15, 15%, 16, 16%, 17, 17%, 18, 18%, 19, 19%, 20, 20%, 21, 21%, 22, 22%, 23, 23%, 24, 24%, 25, 25%, 26, 26%, or more) weeks after the immediately preceding dose. The phrase “the immediately preceding dose,” as used herein, means, in a sequence of multiple administrations, the dose of an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32) which is administered to a patient prior to the administration of the very next dose in the sequence with no intervening doses.

The methods according to this aspect of the disclosure may comprise administering to a patient any number of secondary and/or tertiary doses of an ATDC that binds specifically to GLP1R (e.g., an ATDC which is REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280, e.g., having a linker which is LP11, LP30 or LP32). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples illustrate specific aspects of the instant description. The examples should not be construed as limiting, as the examples merely provide specific understanding and practice of the embodiments and their various aspects.

The abbreviations used in the Examples and throughout the specification are as follows:

| Abbreviation | Term |
|---|---|
| aa# (e.g., aa1) | amino acid number (e.g., amino acid 1) |
| Ac | acetyl |
| ADC | antibody-drug conjugation |
| aq. | aqueous |
| Boc | t-butoxycarbonyl |
| CD | cyclodextrin |
| DCC | dicyclohexylcarbodiimide |
| DCM | dichloromethane |
| DIPEA | diisopropylethylamine |
| DMAP | 4-Dimethylaminopyridine |
| DMF | N,N-Dimethylformamide |
| DMSO | dimethylsulfoxide |
| EDCl | 1-ethyl-3-(3-dimethylaminopropyl)carbodiimide hydrochloride |
| Et | ethyl |
| EtOAc | Ethyl acetate |
| EtOH | ethanol |
| Et₃N | Triethylamine |
| Fmoc | 9-fluorenylmethoxycarbonyl |
| FmocCl | 9-Fluorenylmethyl chloroformate |
| FmocOSu | N-(9-Fluorenylmethoxycarbonyloxy) succinimide |
| HATU | O-(7-azabenzotriazol-1-yl)-NV,NV,N′,N′-tetramethyluronium |
| | hexafluorophosphate |
| HOBt (HOBT) | 1-hydroxybenzotriazole |
| HOSu | N-hydroxysuccinimide |
| HPLC | high-pressure liquid chromatography |
| HRMS | High-resolution mass spectrometry |
| LCMS | Liquid chromatography-mass spectrometry |
| Me | methyl |
| MPM (PMB) | p-methoxybenzyl |
| Ms | mesyl (methanesulfonyl) |
| MS | mass spectrometry |
| 4A MS | 4A molecular sieves |
| MW | Molecule weight |
| MeOH | methanol |
| NMR | nuclear magnetic resonance |
| PEG | polyethylene glycol |
| Ph | phenyl |
| Pr | propyl |
| psi | pounds per square inch |
| Py (pyr) | pyridine |
| PE | Petroleum ether |
| Resin | MBHA resin (0.3~0.8 mmol/g, 100~200 mesh, 1% DVB) |
| R_{f} | retention factor in chromatography |
| t-Bu (tBu) | tert-butyl |
| t-BuOMe (MTBE, TBME) | Methyl tert-butyl ether |
| TEA | triethylamine |
| TES | triethylsilyl |
| TFA | trifluoroacetic acid |
| Tfa | trifluoroacetamide |
| THF | tetrahydrofuran |
| Tr (Trt) | trityl (triphenylmethyl) |
| TRTCl | triphenylmethyl chloride |
| Ts (Tos) | p-toluenesulfonyl |
| Rink Amide Linker | 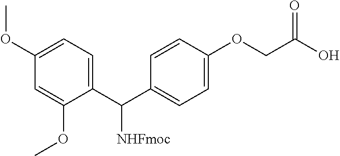 |
| Fmoc - Rink Amide MBHA Resin | 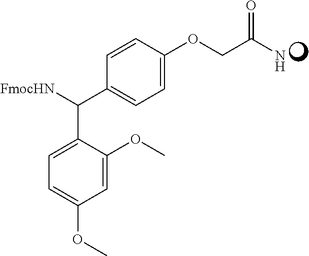 |
| Rink Amide MBHA Resin | 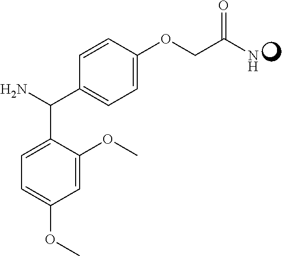 [SMILES: COc1ccc(C(N)c2ccc(OCC(N)=O)cc2)c(OC)c1] |
| DIBAC-PEG₄-acid | 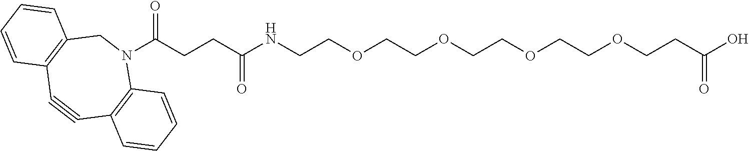 [SMILES: O=C(O)CCOCCOCCOCCOCCNC(=O)CCC(=O)N1Cc2ccccc2C#Cc2ccccc21] |
| DIBAC-PEG₄-NHS | 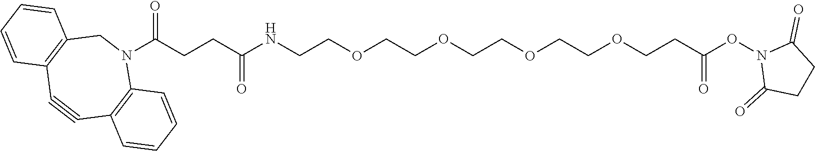 [SMILES: O=C(CCC(=O)N1Cc2ccccc2C#Cc2ccccc21)NCCOCCOCCOCCOCCC(=O)ON1C(=O)CCC1=O] |
| DIBAC-PEG₈-acid | 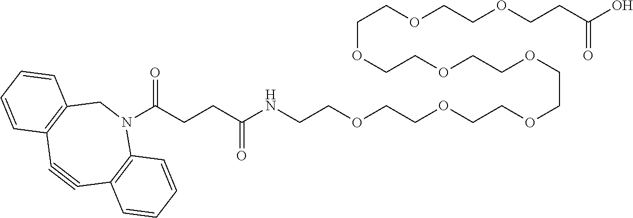 [SMILES: O=C(O)CCOCCOCCOCCOCCOCCOCCOCCOCCNC(=O)CCC(=O)N1Cc2ccccc2C#Cc2ccccc21] |
| DIBAC-PEG₈-NHS | 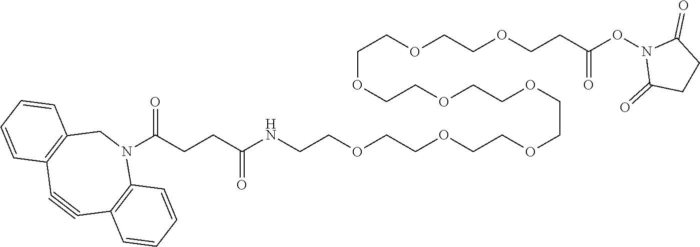 [SMILES: O=C(CCC(=O)N1Cc2ccccc2C#Cc2ccccc21)NCCOCCOCCOCCOCCOCCOCCOCCOCCC(=O)ON1C(=O)CCC1=O] |
| DIBAC-PEG₁₂-acid | 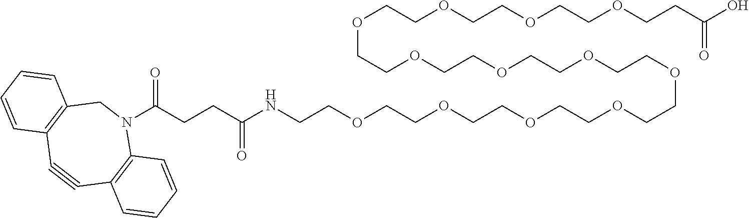 [SMILES: O=C(O)CCOCCOCCOCCOCCOCCOCCOCCOCCOCCOCCOCCOCCNC(=O)CCC(=O)N1Cc2ccccc2C#Cc2ccccc21] |
| DIBAC-PEG₁₂-NHS | 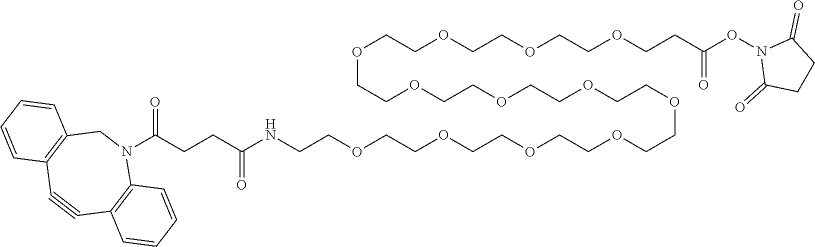 [SMILES: O=C(CCC(=O)N1Cc2ccccc2C#Cc2ccccc21)NCCOCCOCCOCCOCCOCCOCCOCCOCCOCCOCCOCCOCCC(=O)ON1C(=O)CCC1=O] |
| DIBAC-PEG₂₄-acid | 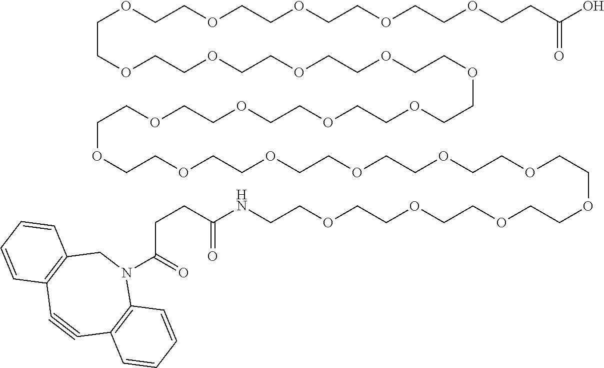 [SMILES: O=C(O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N1Cc2ccccc2C#Cc2ccccc21] |
| DIBAC-PEG₂₄-NHS | 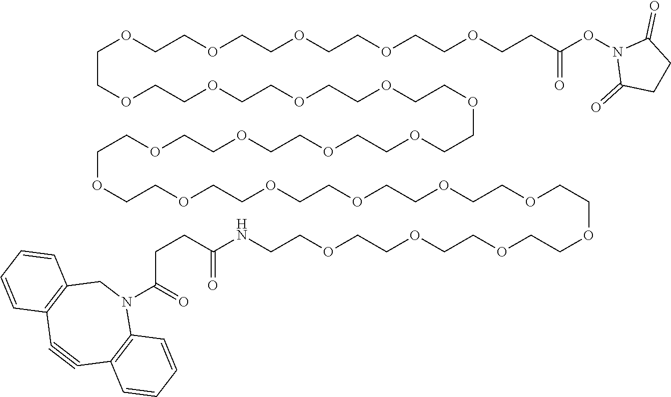 [SMILES: O=C(CCC(=O)N1Cc2ccccc2C#Cc2ccccc21)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O] |
| Azido-DIBAC-PEG₂₄- linker | 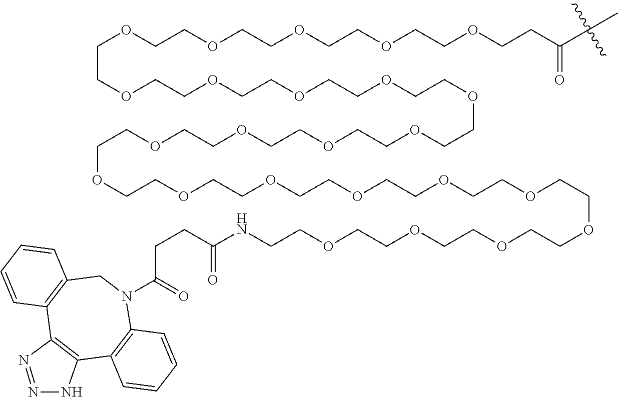 [SMILES: *C(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N1Cc2ccccc2-c2nn[nH]c2-c2ccccc21] |
| CD-N₃ | 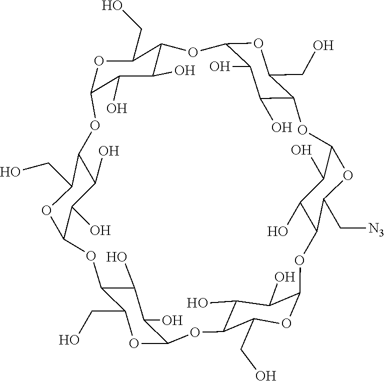 [SMILES: [N-]=[N+]=NCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O] |
| CD-N₃-DIBAC-PEG₂₄- linker | 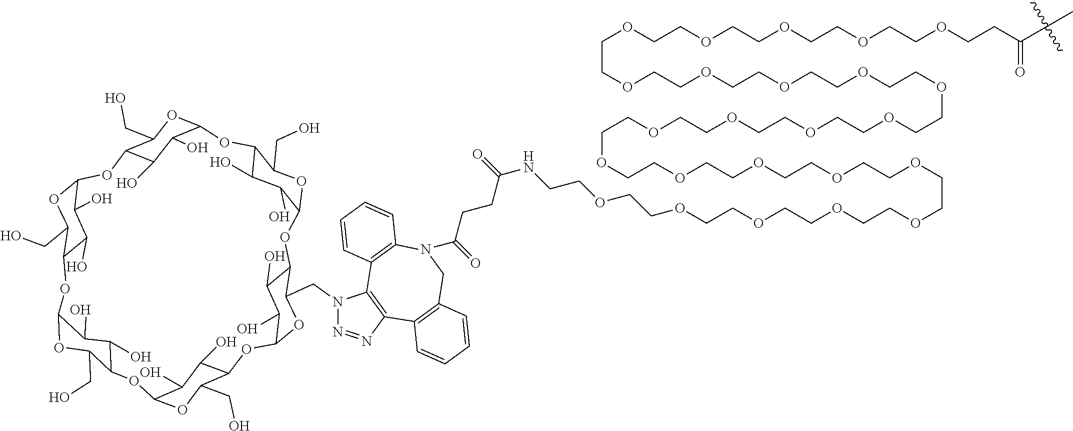 |

### Example 1. Synthesis of Small Molecular Payloads and Linker-Payloads

### 1.1 Solid Phase Peptide Synthesis of Peptidomimetic Payloads

### General Procedure of Preparation of Peptidomimetics (Payloads) Using SPPS Approach

Scheme 1 depicts an assembly of peptidomimetic payloads according to the disclosure on resin. The peptides were assembled manually by a roller-mixer onto Fmoc SPPS (Solid phase peptide synthesis) using polypropylene columns equipped with a filter disc. A sufficient quantity of Rink amide MBHA resin (loading: 0.5-0.6 mmol/g) was swollen in DMF or CH₂Cl₂ for 15 min.

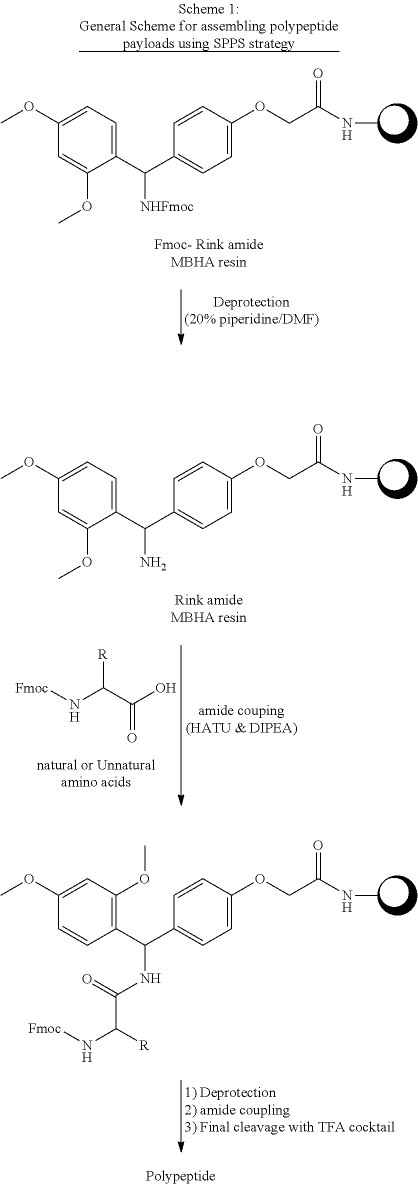

Step 1: General Procedure for Removal of Fmoc from Fmoc-Rink Amide MBHA Resin

The Fmoc-group on the resin was removed by incubation of resin with 20% piperidine in DMF (10-30 ml/100 mg of resin) for 5 to 15 min. The deprotected resin was filtered and washed with excess of DMF and DCM. After washing three times, the resin was incubated in a freshly distilled DMF (1 mL/100 mg of resin), under nitrogen atmosphere for 5 min.

### Step 2: General Procedure for Amide Coupling on Rink Amide MBHA Resin

For the amide coupling reaction with the SPPS-reactant, a DMF solution containing HATU (1.5-4 eq.), Fmoc-protected amino acid (1.5-5 eq. at 0.5M concentration), and DIPEA (5-10 eq.) were added to the resin. For the amide coupling reaction with a natural amino acid as a reactant, the Fmoc-amino acid (5 eq.), HATU (4.5 eq.) and DIPEA (10 eq.) were mixed with the resin; for the amide coupling reaction with an unnatural amino acid as a reactant, the Fmoc-amino acid (1.5-2 eq.), HATU (1.5 eq.) and DIPEA (5.0 eq.) were mixed with the resin. The mixed resin mixture was then shaken for 1-3 hours under nitrogen atmosphere, and the coupling reactions were monitored using a ninhydrin test qualitative analysis. After attachment of the Fmoc-protected amino acid, the resin was then washed with DMF and DCM to generate the corresponding peptide bound resin.

Step 3: General Procedure for Cleavage from Resin Followed by Global Deprotection

The resin-bound peptidomimetic payloads were subjected to cleavage and deprotection with TFA cocktail as follows. A solution of TFA/water/triisopropylsilane (95:2.5:2.5) (10 mL per 100 mg of peptidyl-resin) was added to peptidyl-resins and the mixture was kept at room temperature. After 2-3 hours, the resin was filtered and rinsed by a cleavage solution. The combined filtrate was treated with cold t-BuOMe to precipitate the peptide. The suspension was centrifuged for 10 min (5000 R). The crude white powder was combined and purified by preparative HPLC.

The peptide chain elongation was performed by a number of iterations consisting of deprotection, washing, coupling, and washing procedures, (i.e. the resin was subjected to the reaction conditions for 1 hour each time, and the solution was drained and the resin was re-subjected to fresh reagents each time), as depicted in Scheme 1. Finally, the resulting Fmoc-protected peptidyl-resin was deprotected by 20% piperidine as described above and washed with DMF and DCM four times each. The resin bound peptide was dried under nitrogen flow for 10-15 minutes and subjected to cleavage/deprotection. Using the above protocol and suitable variations thereof, the peptidomimetics designed in the present disclosure were prepared, using Fmoc-SPPS approach. Furthermore, the resin bound peptidomimetics were cleaved and deprotected, purified and characterized using the following protocol.

### 1.2 Preparative HPLC Purification of the Crude Peptidomimetics:

The preparative HPLC was carried out on a Shimadzu LC-8a Liquid chromatograph. A solution of crude peptide dissolved in DMF or water was injected into a column and eluted with a linear gradient of ACN in water. Different methods were used. (See General Information). The desired product eluted were in fractions and the pure peptidomimetics were obtained as amorphous with powders by lyophilization of respective HPLC fractions. In general, after the prep-HPLC purification, the overall recovery was found to be in the range of 40-50% yield.

Preparative HPLC method A: using FA condition (column: Xtimate C18 150*25 mm*5 μm; mobile phase: [water (0.225% FA)-ACN]; B %: 40%-70%, 7 min) to afford a pure product.

Preparative HPLC method B: using TFA condition (column: YMC-Exphere C18 10 μm 300*50 mm 12 nm; mobile phase: [water (0.1% TFA)-ACN]; B %: 15%-45%, 55 min) to afford a pure product.

Preparative HPLC method C: using neutral condition (column: Phenomenex Gemini-NX 150*30 mm*5 μm; mobile phase: [water (10 mM NH₄HCO₃)-ACN]; B %: 21%-51%, 11 min) to afford a pure product.

Preparative HPLC method D: using neutral condition (column: Waters Xbridge 150*255u; mobile phase: [water (10 mM NH₄HCO₃)-ACN]; B %: 20%-50%, 7 min) to afford a pure product.

Preparative HPLC method E: using FA condition (column: Phenomenex Luna C18 250*50 mm*10 μm; mobile phase: [water (0.225% FA)-ACN]; B %: 55%-86%, 21 min) to afford a pure product.

### 1.3 HPLC Analysis of the Purified Peptidomimetics:

After purification by preparation HPLC as described above, each peptide was analyzed by analytical HPLC with using methods A, B, C, D, E, or F. The acquisition of chromatogram was carried out at 220 nm, using a PDA detector, in general, the purity of pure peptidomimetics obtained after Prep-HPLC purification was found to be >95%.

HPLC method A (20 min): Mobile Phase: 4.0 mL TFA in 4 L water (solvent A) and 3.2 mL TFA in 4 L acetonitrile (solvent B), using the elution gradient 10%-80% (solvent B) over 20 minutes and holding at 80% for 3.5 minutes at a flow rate of 1.0 mL/minutes; Column: Gemini-NX 5 μm 150*4.6 mm, C18, 110A Wavelength: UV 220 nm, 254 nm; Column temperature: 30° C.

HPLC method B (15 min): Mobile Phase: 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 mL/min; Column: WELCH Ultimate LP-C18 150*4.6 mm 5 μm; Wavelength: UV 220 nm, 215 nm, 254 nm; Column temperature: 40° C.

HPLC method C (8 min): Mobile Phase: 2.75 mL/4 L TFA in water (solvent A) and 2.5 mL/4 L TFA in acetonitrile (solvent B), using the elution gradient 10%-80% (solvent B) over 7 minutes and holding at 80% for 0.48 minutes at a flow rate of 1.5 mL/min; Column: Ultimate XB-C18.3 μm, 3.0*50 mm; Wavelength: 220 nm, 215 nm, 254 nm; Column temperature: 40° C.

HPLC method D (15 min): Mobile Phase: water containing 0.04% TFA (solvent A). and acetonitrile containing 0.02% TFA (solvent B), using the elution gradient 10% to 80% (solvent B) over 15 minutes and holding at 80% for 3.5 minutes at a flow rate of 1.5 mL/minutes; Column: YMC-Pack ODS-A 150*4.6 mm Wavelength: UV 220 nm, 254 nm; Column temperature: 30° C.

HPLC method E (8 min): Mobile Phase: 0.2 mL/1 L NH3*H₂O in water (solvent A) and acetonitrile (solvent B), using the elution gradient 0%-60% (solvent B) over 5 minutes and holding at 60% for 2 minutes at a flow rate of 1.2 ml/min; Column: Xbridge Shield RP-18, 5 μm, 2.1*50 mm. Wavelength: UV 220 nm, 254 nm; Column temperature: 30° C.

HPLC method F (7 min): Mobile Phase: 1.5 mL/4 L TFA in water (solvent A) and 0.75 mL/4 L TFA in acetonitrile (solvent B), using the elution gradient 10%-80% (solvent B). Column: Xtimate C18 2.1*30 mm; Wavelength: UV 220 nm, 254 nm; Column temperature: 50° C.

### 1.4 Characterization by Mass Spectrometry:

Each peptide was characterized by electrospray ionization mass spectrometry (ESI-MS), either in flow injection or LC/MS mode. In all cases, the experimentally measured molecular weight was within 0.5 Daltons of the calculated monoisotopic molecular weight. Using the above described protocol, all the crude/pure peptidomimetics were characterized by mass spectroscopy and in general, observed mass of peptidomimetic agreed with the calculated/theoretical mass, which confirms successful synthesis of desired peptidomimetics.

LC-MS method A: a MERCK (RP-18e 25-2 mm) column, with a flow rate of 1.5 mL/min, eluting with a gradient of 5% to 95% acetonitrile containing 0.02% TFA (solvent B) and water containing 0.04% TFA (solvent A).

LC-MS method B: a Xtimate (C18 2.1*30 mm, 3 μm) column, with a flow rate of 0.8 mL/min, eluting with a gradient of 10% to 80% acetonitrile containing 0.02% TFA (solvent B) and water containing 0.04% TFA (solvent A).

LC-MS method C: a Chromolith (Flash RP-18e 25-3 mm) column, with a flow rate of 1.5 mL/min, eluting with a gradient of 5% to 95% acetonitrile containing 0.04% TFA (solvent B) and water containing 0.06% TFA (solvent A).

LC-MS method D: Agilent, a Pursuit (5 C18 20*2.0 mm) column, flow rate 1.5 mL/min, eluting with a gradient of 5% to 95% acetonitrile containing 0.02% TFA (solvent B) and water containing 0.04% TFA (solvent A).

LC-MS method E: Waters Xbridge C18 30*2.0 mm, 3.5 μm column, with a flow rate of 1.0 mL/min, eluting with a gradient of 5% to 95%. Mobile phase: A) 0.05% NH₃H₂O in Water; B) ACN. Gradient: 0% B increase to 95% B within 5.8 min; hold at 95% B for 1.1 min; then back to 0% B at 6.91 min and hold for 0.09 min.

LC-MS method F: XBridge C18 3.5 μm 2.1*30 mm Column, with a flow rate of 1.0 mL/min, Mobile phase: 0.8 mL/4 L NH₃·H₂O in water (solvent A) and acetonitrile (solvent B), using the gradient 10%-80% (solvent B) over 2 minutes and holding at 80% for 0.48 minutes.

1.5 HRMS Analysis was Performed on an Aqilent 6200 Series TOF and 6500 Series Q-TOF LC/MS System.

The mobile phase: 0.1% FA in water (solvent A) and ACN (solvent B); Elution Gradient: 5%-95% (solvent B) over 3 minutes and holding at 95% for 1 minute at a flow rate of 1 ml/minute; Column: Xbridge Shield RP 18 5 μm, 2.1*50 mm Ion Source: AJS ESI source; Ion Mode: Positive; Nebulization Gas: Nitrogen; Drying Gas (N2) Flow: 8 L/min; Nebulizer Pressure: 35 psig; Gas Temperature: 325° C.; Sheath gas Temperature: 350° C.; Sheath gas flow: 11 L/min; Capillary Voltage: 3.5 KV; Fragmentor Voltage: 175 V.

### Example 2. Synthesis of Unnatural Amino Acids

TABLE 1
Structures of natural and unnatural amino acids aa1 - aa30

| | | | | ESI-MS | |
|---|---|---|---|---|---|
| | | | | Calcd. | |
| No. | Structure | Source* | MF | M/Z | Found |
| aa1 | 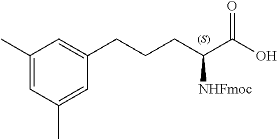 | GB2551945a Jazayeri, A. et al. Nature. 2017, 546, 254-258 | C₂₈H₂₉NO₄ | 443.2 | 466.1 [M + Na]⁺ |
| aa1b | 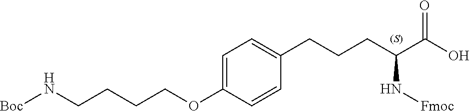 | Prepared | C₃₅H₄₂N₂O₇ | 602.2 | 625.1 [M + Na]⁺ |
| aa2 | 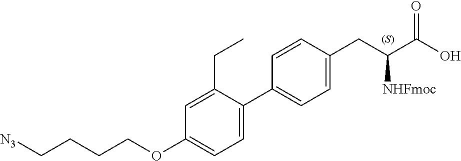 | Prepared | C₃₆H₃₆N₄O₅ | 604.2 | 627.3 [M + Na]⁺ |
| aa2b | 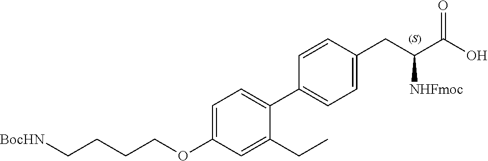 | Prepared | C₄₁H₄₆N₂O₇ | 678.3 | 701.3 [M + Na]⁺ |
| aa2c | 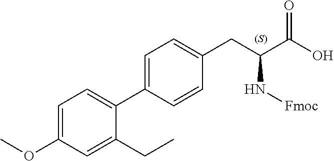 | Prepared | C₄₀H₄₃N₂O₇ | 662.77 | 663.5 [M + H]⁺ |
| aa3 | 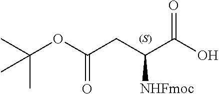 | Commercial | | | |
| aa4 | 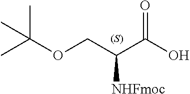 | Commercial | | | |
| aa5 | 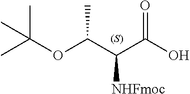 | Commercial | | | |
| aa6 | 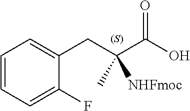 | Commercial | | | |
| aa7 | 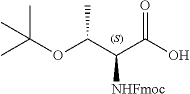 | Commercial | | | |
| aa8 | 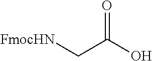 | Commercial | | | |
| aa9 | 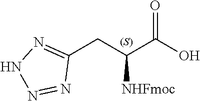 | Ceretti, S. et al Eur. J. Org. Chem. 2004, 4188-4196 | C₁₉H₁₇N₅O₄ | 379.1 | 402.0 [M + Na]⁺ |
| aa9a | 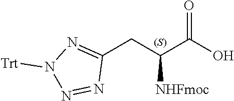 | Prepared | C₃₈H₃₁N₅O₄ | 621.2 | |
| aa9d | 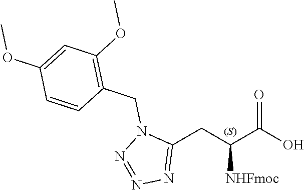 | Prepared | C₂₈H₂₇N₅O₆ | 529.20 | 530.4 [M + H]⁺ |
| aa10 | 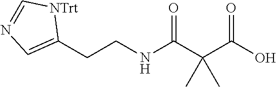 | GB2551945a | C₂₉H₃₀N₃O₃ | 467.2 | 468.1 [M + H]⁺ |
| aa11 | 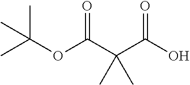 | Commercial | | | |
| aa12 | 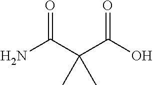 | WO2010/052253 | | | |
| aa13 | 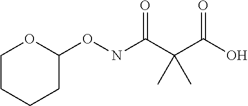 [SMILES: CC(C)(C(=O)O)C(=O)NOC1CCCCO1] | Prepared | | | |
| aa14 | 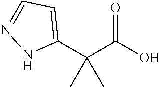 | Prepared | C₇H₁₁N₂O₂ | 154.1 | 155.0 [M + H]⁺ |
| aa15 | 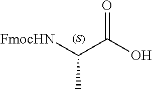 | Commercial | | | |
| aa16 | 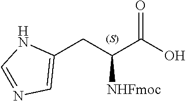 | Commercial | | | |
| aa17 | 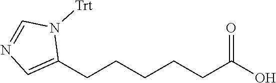 | US2003/114668 | C₂₈H₂₇N₂O₂ | 422.22 | 423.2 [M − H]⁻ |
| aa18 | 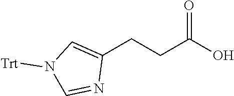 | Crich, D. et al, Org. Lett. 2007, 9, 4423- 4426 | C₂₅H₂₃N₂O₂ | 382.18 | 383.2 [M + H]⁺ |
| aa19 | 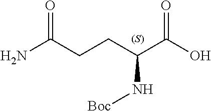 | Commercial | | | |
| aa20 | 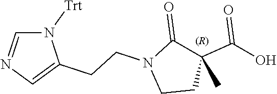 | Prepared | C₃₀H₃₀N₃O₃ | 479.2 | 480.3 [M + H]⁺ SFC- HPLC: RT = 2.346 min |
| aa21 | 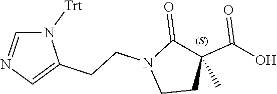 | Prepared | C₃₀H₃₀N₃O₃ | 479.2 | 480.3 [M + H]⁺ SFC- HPLC: RT = 3.607 min |
| aa22 | 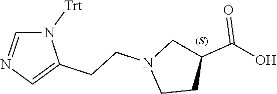 | Prepared | C₂₉H₃₀N₃O₂ | 451.2 | 452.3 [M + H]⁺ |
| aa23 | 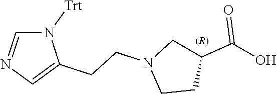 | Prepared | C₂₉H₃₀N₃O₂ | 451.2 | 452.2 [M + H]⁺ |
| aa24 | 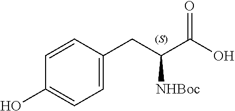 | Commercial | | | |
| aa25 | 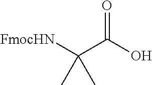 | Commercial | | | |
| aa26 | 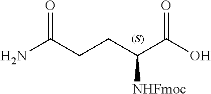 | Commercial | | | |
| aa27 | 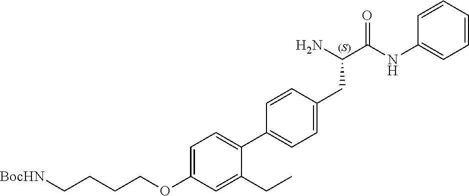 | Prepared | C₃₂H₄₁N₃O₄ | 531.3 | 554.1 [M + Na]⁺ |
| aa28 | 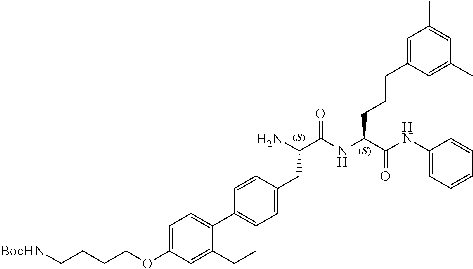 | Prepared | C₄₅H₅₈N₄O₅ | 734.4 | 757.5 [M + Na]⁺. |
| aa29 | 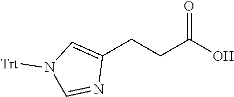 | Prepared | C₂₅H₂₂N₂O₂ | 382.17 | 381.17 [M − H]⁻ |
| aa30 | 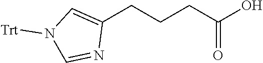 | Prepared | C₂₆H₂₄N₂O₂ | 396.18 | 397.20 [M − H]⁻ |
| aa31 | 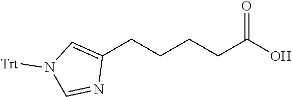 | Prepared | C₂₇H₂₆N₂O₂ | 410.20 | 411.3 [M − H]⁻ |
| aa32 | 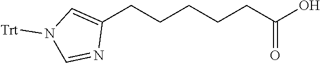 | Prepared | C₂₈H₂₈N₂O₂ | 424.22 | 425.22 [M − H]⁻ |
| aa33 | 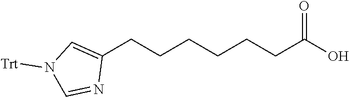 | Prepared | C₂₉H₃₀N₂O₂ | 438.23 | 439.23 [M − H]⁻ |
| aa34 | 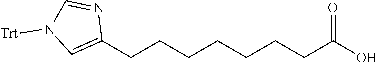 | Prepared | C₃₀H₃₂N₂O₂ | 452.25 | 453.23 [M − H]⁻ |
| aa35 | 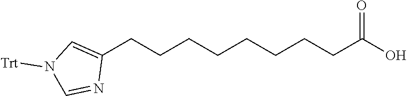 | Prepared | C₃₁H₃₄N₂O₂ | 466.26 | 465.25 [M − H]⁻ |
| aa36 | 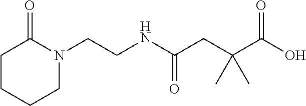 [SMILES: CC(C)(CC(=O)NCCN1CCCCC1=O)C(=O)O] | Prepared | C₁₃H₂₂N₂O₄ | 270.16 | 271.2 [M + H]⁺ |
| aa37 | 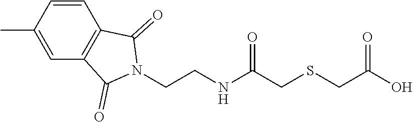 [SMILES: Cc1ccc2c(c1)C(=O)N(CCNC(=O)CSCC(=O)O)C2=O] | Prepared | C₁₅H₁₆N₂O₅S | 336.08 | 358.9 [M + Na]⁺ |
| aa38 | 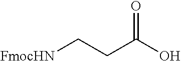 | Commercial | C₁₈H₁₇NO₄ | 311.12 | / |
| aa39 | 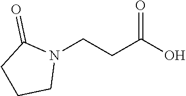 [SMILES: O=C(O)CCN1CCCC1=O] | Commercial | C₇H₁₁NO₃ | 157.07 | / |

2.1 Synthesis of Unnatural Amino Acid (aa1b)
Scheme 2 outlines the synthesis of unnatural amino acid (aa1b):

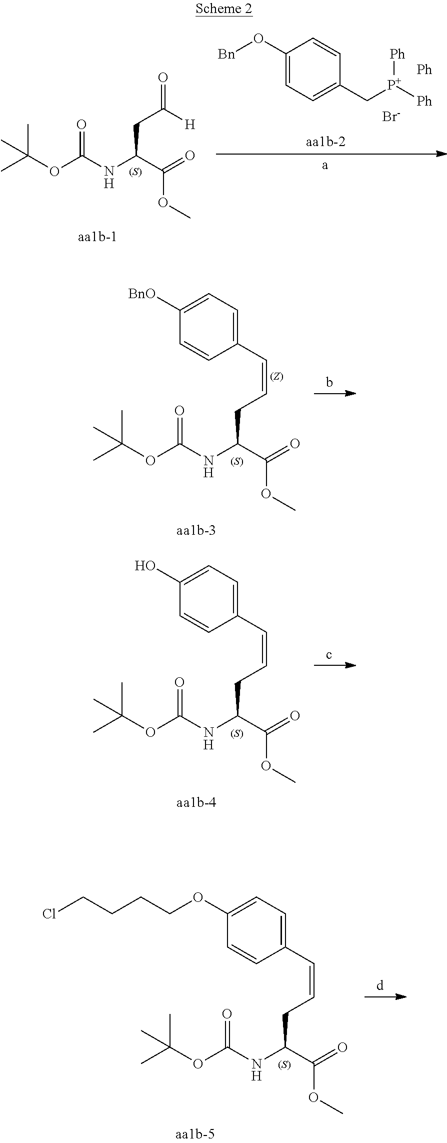

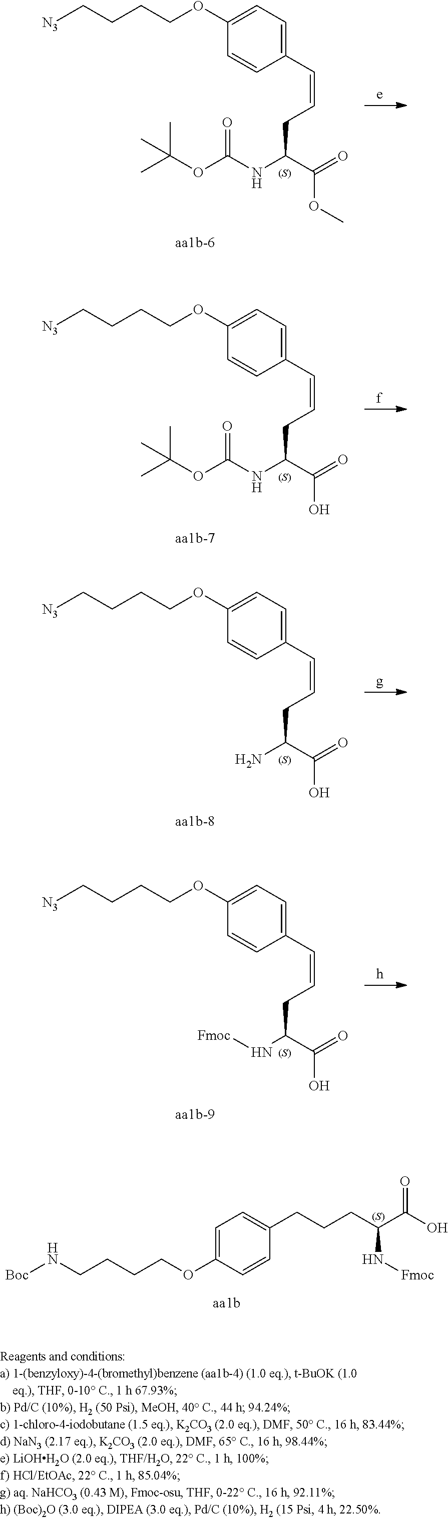

Aa1b-2 were prepared according to the detailed synthetic procedure found in Wu, X. Y.; Stockdill, J. L.; Park, P. K.; Samuel J. Danishefsky, S. J. Expanding the Limits of Isonitrile-Mediated Amidations: On the Remarkable Stereosubtleties of Macrolactam Formation from Synthetic Seco-Cyclosporins. J. Am. Chem. Soc. 2012, 134, 2378-2384. Preparation of aa1b-1 was referred to at Du, J. J.; Gao, X. F.; Xin, L. M.; Lei, Z.; Liu, Z.; and Guo, J. Convergent Synthesis of N-Linked Glycopeptides via Aminolysis of w-Asp p-Nitrophenyl Thioesters in Solution. Org. Lett. 2016, 18, 4828-4831.

### Step 1: Synthesis of (S,Z)-methyl 5-(4-(benzyloxy)phenyl)-2-((tert-butoxycarbonyl)amino)pent-4-enoate (aa1b-3)

To a suspension of aa1b-2 (17.50 g, 32.43 mmol, 1.0 eq.) in THF (100 mL) was added t-BuOK (3.64 g, 32.43 mmol, 1.0 eq.) under nitrogen at 0° C. The mixture was stirred at 0° C. for 30 min. A solution of aa1b-1 (7.5 g, 32.43 mmol, 1.0 eq.) in THF (50 mL) was added dropwise to the mixture. The reaction was warmed to 10° C. and stirred for 1 hr. Light yellow suspension was observed. The reaction was added to ice-water (300 mL) and extracted with EtOAc (200 mL×2). The organic layers were combined and washed with brine (200 mL), dried over Na2SO4, filtered and concentrated to give crude as yellow oil. The residue was purified by flash silica gel chromatography (ISCO@; 80 g SepaFlash@ Silica Flash Column, Eluent of 0˜18% Ethylacetate/Petroleum ether gradient @ 50 mL/min) for 1.5 h with total volume 2.5 L to give aa1b-3 (9.7 g, 23.57 mmol, 67.93% yield) as a light yellow oil.

¹H NMR (400 MHz, CHLOROFORM-d) δ=7.45-7.26 (m, 6H), 7.18 (br d, J=8.6 Hz, 1H), 6.92 (dd, J=8.7, 11.4 Hz, 2H), 6.56-6.34 (m, 1H), 5.98-5.83 (m, 1H), 5.55-5.42 (m, 1H), 5.07 (d, J=2.2 Hz, 3H), 4.48-4.36 (m, 1H), 3.77-3.68 (m, 3H), 2.94-2.56 (m, 2H), 1.43 (s, 9H)

### Step 2: Synthesis of (S)-methyl 2-((tert-butoxycarbonyl)amino)-5-(4-hydroxyphenyl) pentanoate (aa1b-4)

A solution of methyl aa1b-3 (9.7 g, 23.57 mmol, 1.0 eq.) and Pd/C (10% palladium on activated carbon, 1.0 g, 9.40 mmol) in MeOH (100 mL) was stirred at 40° C. under 50 Psi of hydrogen for 44 hr. Black suspension was observed. The reaction was filtered through a pad of Celite, the cake was washed with MeOH (50 mL×3). The filtrate was concentrated in vacuum to give aa1b-4 (7.5 g, 22.22 mmol, 94.24% yield, 95.788% purity) as a gray solid.

¹H NMR (400 MHz, CHLOROFORM-d) δ=7.05-6.92 (m, J=8.3 Hz, 2H), 6.79-6.70 (m, J=8.3 Hz, 2H), 5.59 (br s, 1H), 5.03 (br d, J=7.8 Hz, 1H), 4.37-4.26 (m, 1H), 3.71 (s, 3H), 2.61-2.47 (m, 2H), 1.81 (br s, 1H), 1.68-1.54 (m, 3H), 1.44 (s, 9H). LCMS (ESI): RT=0.927 min, mass calcd. for C₁₇H₂₅NO₅ 323.17, m/z found 345.9 [M+Na]⁺. Reverse phase LC-MS was carried out using method C.

### Step 3: Synthesis of (S)-methyl 2-((tert-butoxycarbonyl)amino)-5-(4-(4-chlorobutoxy)phenyl) pentanoate (aa1b-5)

A solution of aa1b-10 (7 g, 21.65 mmol, 1.0 eq.), 1-chloro-4-iodobutane (7.09 g, 32.47 mmol, 1.5 eq.) and K₂CO₃ (5.98 g, 43.29 mmol, 2.0 eq.) in DMF (70 mL) was stirred at 50° C. for 16 hr. The combined reaction was added to ice-water (200 mL) and extracted with EtOAc (150 mL×3). The organic layers were combined and washed with brine (150 mL×2), dried over Na₂SO₄, filtered and concentrated to give crude as yellow oil. The residue was purified by flash silica gel chromatography (ISCO@; 80 g SepaFlash@ Silica Flash Column, Eluent of 0˜20% Ethyl acetate/Petroleum ether gradient @ 50 mL/min) for 1.5 h with total volume 2.5 L to give aa1b-5 (8 g, 19.33 mmol, 83.44% yield) as a colorless oil.

¹H NMR (400 MHz, CHLOROFORM-d) δ=7.04 (d, J=8.6 Hz, 2H), 6.79 (d, J=8.6 Hz, 2H), 4.95 (br d, J=7.3 Hz, 1H), 4.34-4.22 (m, 1H), 3.98-3.92 (m, 2H), 3.70 (s, 3H), 3.60 (t, J=6.2 Hz, 2H), 2.62-2.48 (m, 2H), 2.04-1.84 (m, 4H), 1.83-1.59 (m, 4H), 1.42 (s, 9H)

### Step 4: Synthesis of (S)-methyl 5-(4-(4-azidobutoxy)phenyl)-2-((tert-butoxycarbonyl)amino) pentanoate (aa1b-6)

A mixture of aa1b-5 (7.5 g, 18.12 mmol, 1.0 eq.), NaN₃ (2.56 g, 39.32 mmol, 2.17 eq.), K₂CO₃ (5.01 g, 36.24 mmol, 2.0 eq.) and KI (300.78 mg, 1.81 mmol, 0.1 eq.) in DMF (75 mL) was stirred at 65° C. for 16 hr. The reaction was added to ice-water (200 mL) and extracted with EtOAc (100 mL×3). The organic layers were combined and washed with brine (100 mL×3), dried over Na₂SO₄, filtered and concentrated to give aa1b-6 (7.5 g, 17.84 mmol, 98.44% yield) as a yellow oil.

### Step 5: Synthesis of (S)-5-(4-(4-azidobutoxy)phenyl)-2-((tert-butoxycarbonyl)amino)pentanoic acid (aa1b-7)

A solution of aa1b-6 (7.5 g, 17.84 mmol, 1.0 eq.) and LiOH·H₂O (1 M, 35.67 mL, 2.0 eq.) in THF (70 mL) was stirred at 22° C. for 1 hr. No change was observed. The reaction was concentrated in vacuum to remove THF. The reaction was adjusted to pH=5 with 1N HCl and extracted with EtOAc (10 mL×2). The organic layers were combined and washed with brine (10 mL), dried over Na₂SO₄, filtered and concentrated to give aa1b-7 (7.25 g, 17.84 mmol, 100.00% yield) as a colorless oil.

### Step 6: Synthesis of (S)-2-amino-5-(4-(4-azidobutoxy)phenyl)pentanoic acid hydrochloride (aa1b-8)

A solution of aa1b-7 (7.25 g, 17.84 mmol, 1.0 eq.) in 4M HCl/EtOAc (75 mL) was stirred at 22° C. for 1 hr. The reaction was concentrated in vacuum to give aa1b-8 (5.2 g, 15.17 mmol, 85.04% yield, HCl) as a white solid.

### Step 7: Synthesis of (S)-2-((((9H-fluoren-9-yl)methoxy)carbonyl)amino)-5-(4-(4-azidobutoxy) phenyl)pentanoic acid (aa1b-9)

To a solution of aa1b-8 (5 g, 14.58 mmol, 1.0 eq., HCl) in THF (116 mL) was added NaHCO₃ (2.45 g, 29.17 mmol, 2.0 eq.) in H₂O (58 mL), and then Fmoc-OSu (5.41 g, 16.04 mmol, 1.1 eq.) was added at 0° C. The reaction mixture was stirred at 22° C. for 16 hr. No change was observed. The reaction was adjusted to pH=6 with 1 N HCl and extracted with EtOAc (50 mL×3). The organic layers were combined and washed with brine (50 mL), dried over Na₂SO₄, filtered and concentrated to give crude as yellow oil. The residue was purified by flash silica gel chromatography (ISCO@; 80 g SepaFlash@ Silica Flash Column, Eluent of 0˜5% MeOH/DCM gradient @ 50 mL/min) for 2.5 h with total volume 3 L to give aa1b-9 (7 g, 12.20 mmol, 83.64% yield, 92.113% purity) as a yellow oil.

¹H NMR (400 MHz, CHLOROFORM-d) δ=7.75 (br d, J=7.3 Hz, 2H), 7.60-7.49 (m, 2H), 7.41-7.34 (m, 2H), 7.29 (br t, J=7.5 Hz, 2H), 7.05 (br d, J=8.3 Hz, 2H), 6.78 (br d, J=8.3 Hz, 2H), 5.18 (br d, J=8.3 Hz, 1H), 4.40 (br d, J=6.8 Hz, 3H), 4.21 (br t, J=7.0 Hz, 1H), 3.98-3.88 (m, 2H), 3.34 (t, J=6.5 Hz, 2H), 2.57 (br d, J=6.1 Hz, 2H), 1.98-1.60 (m, 8H)

### Step 8: Synthesis of (S)-2-((((9H-fluoren-9-yl)methoxy)carbonyl)amino)-5-(4-(4-((tert-butoxycarbonyl) amino) butoxy)phenyl)pentanoic acid (aa1b)

A mixture of aa1b-15 (7 g, 13.24 mmol, 1.0 eq.), DIPEA (5.13 g, 39.73 mmol, 6.92 mL, 3.0 eq.) and Boc₂O (8.67 g, 39.73 mmol, 9.13 mL, 3.0 eq.) and Pd/C (10% palladium on activated carbon, 3.5 g, 32.9 mmol) in EtOAc (100 mL) was stirred at 25° C. under 15 PSi of H₂ for 4 hr. Black suspension was observed. The reaction was filtered through a pad of Celite, the cake was washed with EtOAc (50 mL×3). The filtrate was washed with aq. NH₄Cl (100 mL×3), brine (100 mL), dried over Na₂SO₄, filtered and concentrated in vacuum to give crude as colorless oil. The crude was purified by prep-HPLC (column: Phenomenex Luna(2) C18 250*50 10 μm; mobile phase: [water(0.225% FA)-ACN]; B %: 45%-78%, 21.5 min) to give aa1b (1.8 g, 2.99 mmol, 22.50% yield) as a yellow oil. LCMS (ESI): RT=0.954 min, mass calcd. for C₃₅H₄₂N₂O₇Na 625.29, m/z found 625.1 [M+Na]⁺. Reverse phase LC-MS was carried out using method A.

¹H NMR (400 MHz, CHLOROFORM-d) δ=7.75 (br d, J=7.6 Hz, 2H), 7.60-7.55 (m, 2H), 7.38 (br t, J=6.7 Hz, 2H), 7.29 (t, J=7.1 Hz, 2H), 7.04 (br d, J=8.1 Hz, 2H), 6.78 (d, J=7.4 Hz, 2H), 5.24 (br s, 1H), 4.40 (br d, J=6.6 Hz, 2H), 4.23-4.18 (m, 1H), 3.92 (br s, 2H), 3.21-3.07 (m, 2H), 2.61-2.51 (m, 2H), 1.97-1.84 (m, 2H), 1.82-1.54 (m, 8H), 1.43 (s, 9H)

### 2.2 the Synthesis of Unnatural Amino Acid (Aa2)

Scheme 3 outlines the synthesis of unnatural amino acid (aa2):

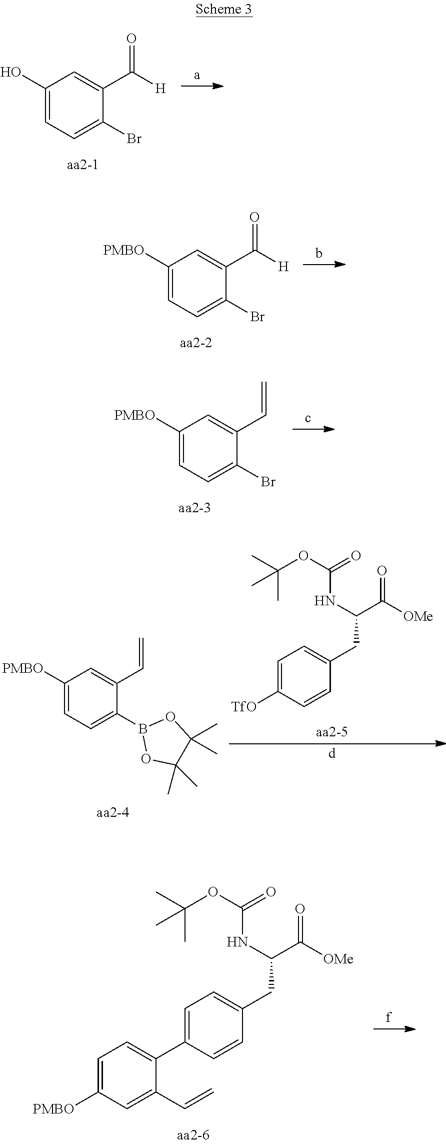

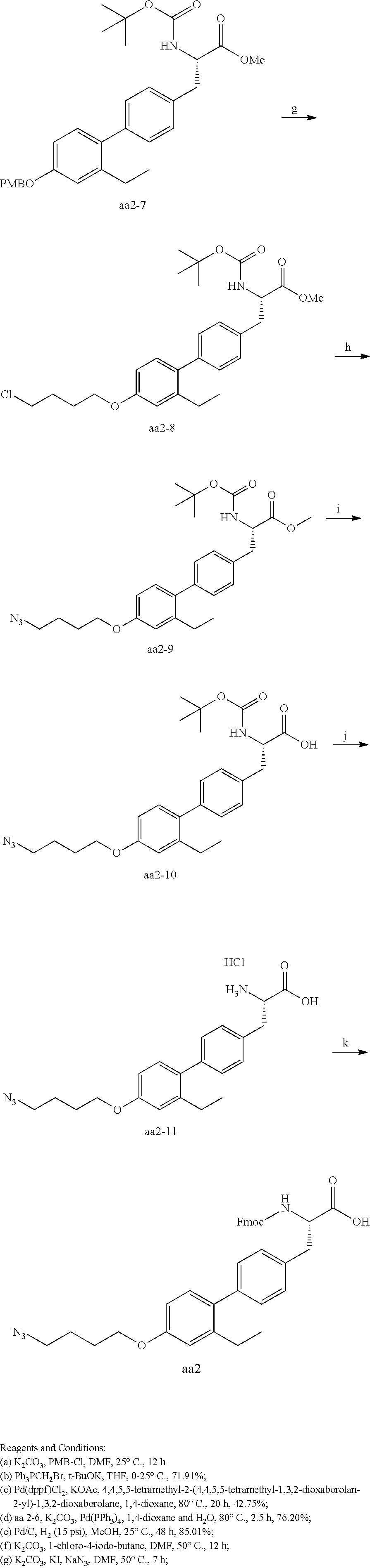

The compound aa2-5 was prepared according to the following literature reference: 1. Berezowska, N. N. Chung, C. Lemieux, B. C. Wilkes, and P. W. Schiller, Agonist vs Antagonist Behavior of 5 Opioid Peptides Containing Novel Phenylalanine Analogues in Place of Tyr. J. Med. Chem., Vol. 52, No. 21, 2009, 6941-6945.

### Step 1: Synthesis of 2-bromo-5-((4-methoxybenzyl)oxy)benzaldehyde (aa2-2)

To a solution of aa2-1 (10 g, 49.75 mmol, 1.0 eq.) and K₂CO₃ (10.31 g, 74.62 mmol, 1.5 eq.) in DMF (100 mL) was added PMB-Cl (11.69 g, 74.62 mmol, 10.16 mL, 1.5 eq.). The mixture was stirred at 25° C. for 12 hr. The reaction mixture was concentrated under reduced pressure. The residue was diluted with H₂O (100 mL) and extracted with EtOAc (100 mL×2). The combined organic layers was dried over anhydrous Na₂SO₄, filtered and concentrated under reduced pressure. Compound aa2-2 (21 g, crude) was obtained as a white solid which was used directly in the next step without any further purification.

¹H NMR (400 MHz, DMSO-d6) δ=10.16 (s, 1H), 7.69 (d, J=8.8 Hz, 1H), 7.43-7.34 (m, 3H), 7.28 (dd, J=3.3, 8.8 Hz, 1H), 6.99-6.90 (m, 2H), 5.10 (s, 2H), 3.75 (s, 3H).

### Step 2: Synthesis of 1-bromo-4-((4-methoxybenzyl)oxy)-2-vinylbenzene (aa2-3)

To a solution of Ph₃PCH₃Br (4.56 g, 12.77 mmol, 1.0 eq.) in THF (25 mL) was added t-BuOK (7.16 g, 63.83 mmol, 5.0 eq.) under nitrogen. The mixture was stirred for 1 hr at 0° C. Then aa2-2 (4.1 g, 12.77 mmol, 1.0 eq.) in THF (25 mL) was added dropwise. The mixture was stirred for 3 hr at 25° C. The reaction mixture was diluted with H₂O (100 mL) and extracted with EtOAc (100 mL×2). The combined organic layers were dried over anhydrous Na₂SO₄, filtered and concentrated under reduced pressure. The residue was purified by flash silica gel chromatography (ISCO@; 40 g SepaFlash@ Silica Flash Column, Eluent of 0˜5% Ethyl acetate/Petroleum ether gradient @ 30 mL/min) for 24 min with total volume 0.9 L. Compound aa2-3 (2.93 g, 9.18 mmol, 71.91% yield) was obtained as a white solid.

¹H NMR (400 MHz, CHLOROFORM-d) δ=7.46-7.29 (m, 3H), 7.18-7.09 (m, 1H), 7.06-6.96 (m, 1H), 6.95-6.86 (m, 2H), 6.81-6.70 (m, 1H), 5.72-5.59 (m, 1H), 5.40-5.29 (m, 1H), 5.03-4.90 (m, 2H), 3.86-3.74 (m, 3H).

### Step 3: Synthesis of 2-(4-((4-methoxybenzyl)oxy)-2-vinylphenyl)-4,4,5,5-tetramethyl-1,3,2-dioxaborolane (aa2-4)

A solution of aa2-3 (200 mg, 626.58 μmol, 1 eq.) in 1,4-dioxane (3 mL) was treated with Pd(PPh₃)₄ (72.41 mg, 62.66 μmol, 0.1 eq.) and KOAc (122.99 mg, 1.25 mmol, 2 eq.), and then 4,4,5,5-tetramethyl-2-(4,4,5,5-tetramethyl-1,3,2-dioxaborolan-2-yl)-1,3,2-dioxaborolane (477.34 mg, 1.88 mmol, 3 eq.) was added. The mixture was stirred at 80° C. for 12 hr under nitrogen. The reaction mixture was diluted with brine (30 mL) and extracted with EtOAc (30 mL×2). The combined organic layers were dried over anhydrous Na₂SO₄, filtered and concentrated under reduced pressure. The residue was purified by flash silica gel chromatography (ISCO@; 4 g SepaFlash@ Silica Flash Column, Eluent of 0˜10% Ethyl acetate/Petroleum ether gradient @ 18 mL/min). Compound aa2-4 (190 mg, 518.76 μmol, 82.79% yield) was obtained as a white solid.

¹H NMR (400 MHz, CHLOROFORM-d) δ=7.75 (d, J=8.4 Hz, 1H), 7.55 (dd, J=10.9, 17.5 Hz, 1H), 7.36 (d, J=8.6 Hz, 2H), 7.22 (d, J=2.4 Hz, 1H), 6.97-6.89 (m, 2H), 6.87 (dd, J=2.4, 8.4 Hz, 1H), 5.67 (d, J=17.4 Hz, 1H), 5.26 (d, J=11.0 Hz, 1H), 5.03 (s, 2H), 3.82 (s, 3H), 1.34 (s, 12H).

### Step 4: Synthesis of (S)-methyl 2-((tert-butoxycarbonyl)amino)-3-(4′-((4-methoxybenzyl)oxy)-2′-vinyl-[1,1′-biphenyl]-4-yl)propanoate (aa2-6)

A solution of aa2-4 (100 mg, 273.03 μmol, 1 eq.) in 1,4-dioxane (3 mL) and H2O (1 mL) was treated with K₂CO₃ (56.60 mg, 409.55 μmol, 1.5 eq.) and Pd(PPh₃)₄ (31.55 mg, 27.30 μmol, 0.1 eq.), and then aa2-5 (116.69 mg, 273.03 μmol, 1 eq.) was added. The mixture was stirred at 80° C. for 2.5 hr under nitrogen. The reaction mixture was diluted with brine (30 mL) and extracted with EtOAc (30 mL×2). The combined organic layers were dried over anhydrous Na₂SO₄, filtered and concentrated under reduced pressure. The residue was purified by flash silica gel chromatography (ISCO@; 4 g SepaFlash@ Silica Flash Column, Eluent of 0˜10% Ethyl acetate/Petroleum ether gradient @ 18 mL/min) for 14 min with total volume 0.3 L. Compound aa2-6 (100 mg, 193.20 μmol, 70.76% yield) was obtained as a yellow oil.

LCMS (ESI): RT=0.950 min, mass calcd. for C₃₁H₃₅NO₆Na 540.24, [M+Na]⁺, m/z found 540.1 [M+Na]⁺. Reverse phase LC-MS was carried out using method A.

¹H NMR (400 MHz, CHLOROFORM-d) δ=7.39 (d, J=8.6 Hz, 2H), 7.27-7.25 (m, 2H), 7.23 (s, 2H), 7.17 (dd, J=8.3, 16.2 Hz, 3H), 6.97-6.91 (m, 3H), 6.67 (dd, J=11.0, 17.4 Hz, 1H), 5.67 (dd, J=1.1, 17.4 Hz, 1H), 5.22-5.15 (m, 1H), 5.05 (s, 2H), 3.83 (s, 3H), 3.74 (s, 3H), 1.47-1.35 (m, 1H), 1.43 (s, 8H).

### Step 5: Synthesis of (S)-methyl 2-((tert-butoxycarbonyl)amino)-3-(2′-ethyl-4′-hydroxy-[1,1′-biphenyl]-4-yl)propanoate (aa2-7)

To a solution of aa2-6 (2.8 g, 5.41 mmol, 1.0 eq.) in MeOH (25 mL) was added Pd/C (300 mg, 10% palladium on activated carbon) and stirred for 48 hr at 25° C. under hydrogen (15 psi). The reaction mixture was filtered and concentrated under reduced pressure. The residue was purified by flash silica gel chromatography (ISCO@; 40 g SepaFlash@ Silica Flash Column, Eluent of 0˜30% Ethyl acetate/Petroleum ether, gradient @ 35 mL/min) for 22 min with total volume 0.9 L. Compound aa2-7 (1.85 g, 4.60 mmol, 85.01% yield, 99.3% purity) was obtained as a colorless oil.

LCMS (ESI): RT=0.935 min, mass calcd. for C₂₃H₂₉NO₅Na 422.20 [M+Na]⁺, m/z found 422.1 [M+Na]⁺. Reverse phase LC-MS was carried out using method A.

¹H NMR (400 MHz, CHLOROFORM-d) δ=7.22-7.11 (m, 4H), 7.04 (d, J=8.2 Hz, 1H), 6.78 (d, J=2.6 Hz, 1H), 6.69 (dd, J=2.6, 8.2 Hz, 1H), 5.24 (s, 1H), 5.05 (br d, J=8.4 Hz, 1H), 4.70-4.58 (m, 1H), 3.73 (s, 3H), 3.20-3.11 (m, 1H), 3.11-3.02 (m, 1H), 2.53 (q, J=7.6 Hz, 2H), 1.42 (s, 9H), 1.08 (t, J=7.5 Hz, 3H).

### Step 6: Synthesis of (S)-methyl 2-((tert-butoxycarbonyl)amino)-3-(4′-(4-chlorobutoxy)-2′-ethyl-[1,1′-biphenyl]-4-yl)propanoate (aa2-8)

To a solution of aa2-7 (350 mg, 876.14 μmol, 1 eq.) and K₂CO₃ (242.18 mg, 1.75 mmol, 2.0 eq.) in DMF (5 mL) was added 1-chloro-4-iodo-butane (287.11 mg, 1.31 mmol, 1.5 eq.) at 25° C. The mixture was stirred at 50° C. for 12 hr. The residue was diluted with brine (50 mL) and extracted with EtOAc (50 mL×2). The combined organic layers were dried over anhydrous Na₂SO₄, filtered and concentrated under reduced pressure. The residue was purified by flash silica gel chromatography (ISCO®; 12 g SepaFlash@ Silica Flash Column, Eluent of 0˜30% Ethyl acetate/Petroleum ether gradient @ 35 mL/min) for 14 min with total volume 0.4 L. Compound aa2-8 (340 mg, crude) was obtained as a yellow oil.

LCMS (ESI): RT=1.145 min, mass calcd. for C₂₇H₃₆ClNO₅Na 512.22 [M+Na]⁺, m/z found 512.2 [M+Na]⁺. Reverse phase LC-MS was carried out using method A.

### Step 7: Synthesis of (S)-methyl 3-(4′-(4-azidobutoxy)-2′-ethyl-[1,1′-biphenyl]-4-yl)-2-((tert-butoxycarbonyl)amino)propanoate (aa2-9)

To a solution of aa2-8 (1.6 g, 3.27 mmol, 1.0 eq.) in DMF (15 mL) was added K₂CO₃ (902.51 mg, 6.53 mmol, 2.0 eq.), KI (54.20 mg, 326.51 μmol, 0.1 eq.) and NaN₃ (460 mg, 7.08 mmol, 2.1 eq.). The mixture was stirred at 50° C. for 7 hr. The reaction mixture was diluted with brine 50 mL and extracted with EtOAc (50 mL×2). The combined organic layers were dried over anhydrous Na₂SO₄, filtered and concentrated under reduced pressure. The water layers were quenched by addition of aqueous NaClO (1.0 M, 100 mL). Compound aa2-9 (1.7 g, crude) was obtained as a yellow oil.

LCMS (ESI): RT=1.140 min, mass calcd. for C₂₇H₃₆N₄O₅Na 519.27, m/z found 519.3 [M+Na]⁺. Reverse phase LC-MS was carried out using method A.

### Step 8: Synthesis of(S)-3-(4′-(4-azidobutoxy)-2′-ethyl-[1,1′-biphenyl]-4-yl)-2-((tert-butoxycarbonyl)amino)propanoic acid (aa2-10)

To a solution of aa2-9 (1.7 g, 3.42 mmol, 1.0 eq.) in THF (12 mL) was added LiOH·H₂O (287.31 mg, 6.85 mmol, 2.0 eq.) in H₂O (6 mL) at 0° C., and then the mixture was allowed to gradually warm to 25° C. and was stirred for 2 hr. The mixture was treated with EtOAc (30 mL) and extracted with water (25 mL×2). The combined aqueous layers were acidified (1 M aqueous HCl) and extracted with EtOAc (50 mL×3). The combined organic layer was dried by anhydrous Na₂SO₄, filtered and concentrated under reduced pressure. Compound aa2-10 (2.01 g, crude) was obtained as a yellow oil which was used directly in the next step without any further purification.

### Step 9: Synthesis of (S)-2-amino-3-(4′-(4-azidobutoxy)-2′-ethyl-[1,1′-biphenyl]-4-yl)propanoic acid hydrochloride (aa2-11)

Compound aa2-10 (2.01 g, 4.17 mmol, 1.0 eq.) was dissolved in 4.0 M HCl/EtOAc (20 mL). The mixture was stirred at 25° C. for 1 hr. The reaction mixture was filtered. The filter cake was washed with EtOAc (30 ml) and dried under vacuum. Compound aa2-11 (1 g, crude) was obtained as a white solid which was used directly in the next step without any further purification.

LCMS (ESI): RT=1.121 min, mass calcd. for C₂₁H₂₇N₄O₃ 383.21 [M+H]⁺, m/z found 383.2 [M+H]⁺. Reverse phase LC-MS was carried out using method A.

### Step 10: Synthesis of (S)-2-((((9H-fluoren-9-yl)methoxy)carbonyl)amino)-3-(4′-(4-azidobutoxy)-2′-ethyl-[1,1′-biphenyl]-4-yl)propanoic acid (aa2)

To a solution of aa2-11 (1 g, 2.39 mmol, 1.0 eq.) in THF (15 mL) was added NaHCO₃ (401.07 mg, 4.77 mmol, 2.0 eq.) in H₂O (8 mL), and then (2,5-dioxopyrrolidin-1-yl) 9H-fluoren-9-ylmethyl carbonate (805.23 mg, 2.39 mmol, 1.0 eq.) was added at 0° C. The mixture was stirred for 12 hr at 25° C. The reaction mixture was diluted with brine (100 mL) and acidified (1 M aqueous HCl) to pH=2-3. The reaction mixture was extracted with EtOAc (30 mL×2). The combined organic layers were dried over anhydrous Na₂SO₄, filtered and concentrated under reduced pressure. The residue was purified by flash silica gel chromatography (ISCO@; 40 g SepaFlash@ Silica Flash Column, Eluent of 0˜10% Methanol/Dichloromethane @ 30 mL/min) for 16 min with total volume 0.6 L. Product aa2 (1.3 g, 2.14 mmol, 89.52% yield, 99.4% purity) was obtained as a white foam.

LCMS (ESI): RT=1.113 min, mass calcd. for C₃₆H₃₆N₄O₅Na 627.26 [M+Na]⁺, m/z found 627.3 [M+Na]⁺. Reverse phase LC-MS was carried out using method A.

¹H NMR (400 MHz, DMSO-d₆) δ=7.88 (d, J=7.5 Hz, 2H), 7.81 (d, J=8.6 Hz, 1H), 7.66 (t, J=6.9 Hz, 2H), 7.40 (dt, J=2.3, 7.3 Hz, 2H), 7.34-7.25 (m, 4H), 7.14 (d, J=8.2 Hz, 2H), 6.97 (d, J=8.4 Hz, 1H), 6.83 (d, J=2.4 Hz, 1H), 6.76 (dd, J=2.5, 8.5 Hz, 1H), 4.27-4.17 (m, 3H), 4.17-4.13 (m, 1H), 4.03-3.98 (m, 2H), 3.42 (t, J=6.7 Hz, 2H), 3.13 (br dd, J=3.9, 13.8 Hz, 1H), 2.96-2.87 (m, 1H), 2.52 (d, J=1.8 Hz, 2H), 2.43 (q, J=7.4 Hz, 2H), 1.82-1.74 (m, 2H), 1.73-1.66 (m, 2H), 0.98-0.90 (m, 3H).

SFC: ee %=97.95%−2.05%=95.9%; Method Comments: Column: Chiralcel OJ-3 100×4.6 mm I.D., 3 μm; Mobile phase: A: C02 B: ethanol (0.05% DEA); Gradient: from 5% to 40% of B in 4 min and hold 40% for 2.5 min, then 5% of B for 1.5 min; Flow rate: 2.8 mL/min; Column temp.: 35° C.; ABPR: 1500 psi.

2.3 the Synthesis of Unnatural Amino Acid (aa2b)

Scheme 4 outlines the synthesis of unnatural amino acid (aa1b):

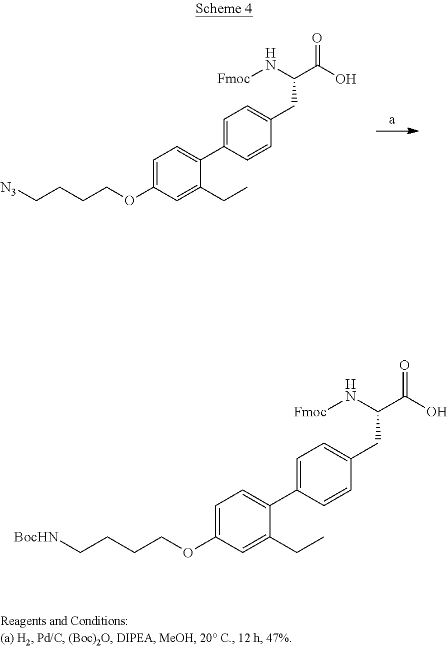

### Step 1: Synthesis of (R)-2-((((9H-fluoren-9-yl) methoxy) carbonyl) amino)-3-(4′-(4-((tert-butoxycarbonyl)amino)butoxy)-2′-ethyl-[1,1′-biphenyl]-4-yl) propanoic acid (aa2b)

To a solution of aa2 (3.2 g, 5.29 mmol, 1.0 eq.) in MeOH (30 mL) was added Pd/C (700 mg, 10% palladium on activated carbon), DIPEA (1.37 g, 10.60 mmol, 1.84 mL, 2.0 eq) and Boc₂O (2.31 g, 10.58 mmol, 2.43 mL, 2.0 eq). The mixture was stirred at 20° C. for 12 hr under hydrogen (15 psi). The reaction mixture was filtered through a small pad of Celite and the cake was rinsed with 40*5 mL of EtOAc (40 mL*5). Then brine (400 mL) was added and extracted with EtOAc (300 mL*2). The combined organic layers were dried over Na₂SO₄, filtered and concentrated under reduced pressure to give a residue. The residue was purified by prep-HPLC (column: Phenomenex Luna C18 250*50 mm*10 um; mobile phase: [water (0.225% FA)-ACN]; B %: 55%-86%, 21 min), the product was suspended in water (150 mL), the mixture frozen in a dry-ice/ethanol bath to afford the product aa2b (3.4 g, 5.01 mmol, 47.32% yield, 100% purity) as a white solid.

LCMS (ESI): RT=0.922 min, mass calcd. for C₄₁H₄₆N₂O₇Na 701.33 [M+Na]⁺, m/z found 701.3 [M+Na]⁺. Reverse phase LC-MS was carried out using method C.

¹H NMR (400 MHz, DMSO-d6) δ=7.87 (d, J=7.6 Hz, 2H), 7.69-7.60 (m, 2H), 7.43-7.36 (m, 2H), 7.33-7.20 (m, 4H), 7.15-7.05 (m, 3H), 6.95 (br d, J=8.3 Hz, 1H), 6.89-6.70 (m, 4H), 4.28 (br dd, J=5.9, 9.0 Hz, 1H), 4.17-4.09 (m, 2H), 4.00-3.91 (m, 3H), 3.19-3.09 (m, 2H), 3.02-2.88 (m, 2H), 2.46-2.38 (m, 2H), 1.73-1.62 (m, 2H), 1.56-1.48 (m, 2H), 1.37 (s, 9H), 0.93 (br t, J=7.6 Hz, 3H).

### 2.4 the Synthesis of Unnatural Amino Acid (Aa13)

Scheme 5 outlines the synthesis of unnatural amino acid (aa13):

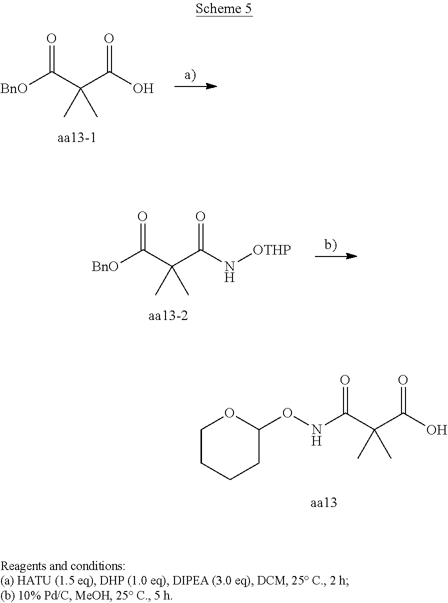

aa13-1 was prepared according to WO2010/052253, the content of which are incorporated by reference herein in their entirety.

### Step 1: Synthesis of benzyl 2,2-dimethyl-3-oxo-3-(((tetrahydro-2H-pyran-2-yl)oxy)amino) propanoate (aa13-2)

To a solution of aa13-1 (200 mg, 899.94 μmol, 1 eq), HATU (513.28 mg, 1.35 mmol, 1.5 eq) and DIPEA (348.93 mg, 2.70 mmol, 470.26 μL, 3 eq) in DCM (10 mL) was stirred at 25° C. for 10 min. Otetrahydropyran-2-ylhydroxylamine (105.42 mg, 899.94 μmol, 1 eq) was added to the mixture and stirred at 25° C. for 2 hr. The reaction was added DCM (5 mL) and washed with aq. NH₄Cl (5 mL). The aqueous layer was separated and extracted with DCM (5 mL). The organic layers were combined and washed with brine (5 mL), dried over anhydrous Na₂SO₄, filtered and concentrated in vacuum to give crude as yellow oil, which was purified by flash silica gel chromatography (ISCO@; 20 g SepaFlash@ Silica Flash Column, Eluent of 0-35% Ethylacetate/Petroleum ethergradient @ 30 mL/min) to give aa13-2 (230 mg, 715.69 μmol, 79.53% yield) as a colorless oil.

¹H NMR (400 MHz, CHLOROFORM-d) δ=9.28-9.14 (m, 1H), 7.38-7.30 (m, 5H), 5.16 (s, 2H), 4.87 (br s, 1H), 3.93-3.80 (m, 1H), 3.59 (br d, J=1.5 Hz, 1H), 1.76 (br s, 3H), 1.65-1.58 (m, 1H), 1.56-1.52 (m, 2H), 1.48 (s, 6H)

### Step 2: Synthesis of 2,2-dimethyl-3-oxo-3-(((tetrahydro-2H-pyran-2-yl)oxy)amino)propanoic acid (aa13)

A black suspension of aa13-2 (230 mg, 715.69 μmol, 1 eq) and 10% Pd/C (23 mg) in MeOH (5 mL) was stirred at 25° C. under 15 Psi of H₂ for 5 hr. The reaction was filtered through a pad of Celite, the cake was washed with MeOH (5 mL*3). The filtrate was concentrated in vacuum to give aa13 (120 mg, 518.93 μmol, 72.51% yield) as colorless oil.

¹H NMR (400 MHz, METHANOL-d4) δ=4.90-4.88 (m, 1H), 4.05 (br s, 1H), 3.59-3.53 (m, 1H), 1.85-1.68 (m, 3H), 1.68-1.51 (m, 3H), 1.40 (s, 6H).

### 2.5 the Synthesis of Unnatural Amino Acid (Aa14)

Scheme 6 outlines the synthesis of unnatural amino acid (aa14):

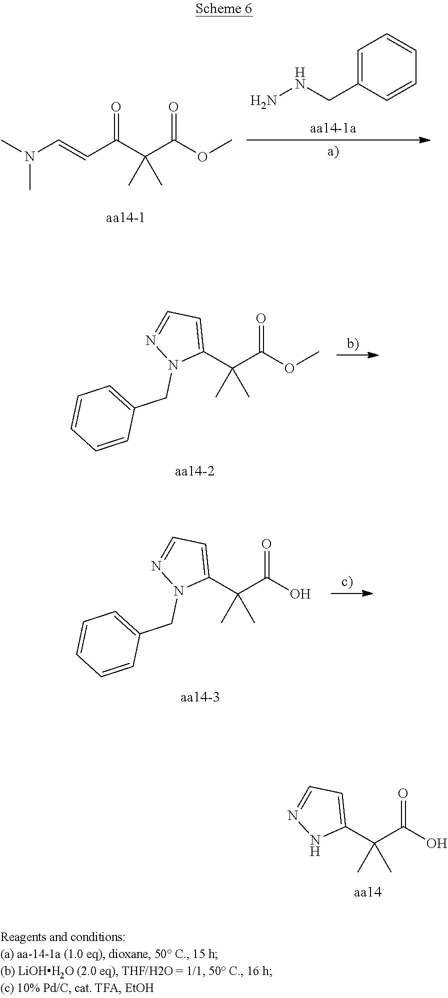

The compound aa14-1 was prepared according to US2015/380666.

### Step 1: Synthesis of methyl 2-(1-benzyl-1H-pyrazol-5-yl)-2-methylpropanoate (aa14-3)

A mixture of aa14-1 (2.76 g, 13.85 mmol, 1 eq) and aa14-1a (2.20 g, 13.85 mmol, 1 eq) in dioxane (30 mL) was stirred at 50° C. for 15 hr. The reaction was filtered and the filtrate was concentrated in vacuum to give crude as brown oil. The residue was purified by flash silica gel chromatography (ISCO@; 20 g SepaFlash@ Silica Flash Column, Eluent of 0˜20% Ethylacetate/Petroleum ether gradient @ 25 mL/min) to give aa14-2 (0.9 g, 3.48 mmol, 25.15% yield) as a light yellow oil.

¹H NMR (400 MHz, CHLOROFORM-d) δ=7.53 (s, 1H), 7.29-7.26 (m, 1H), 7.23-7.16 (m, 1H), 6.98 (d, J=7.4 Hz, 2H), 6.22 (s, 1H), 5.23 (s, 2H), 3.26 (s, 3H), 1.56 (s, 6H)

### Step 2: Synthesis of methyl 2-(1-benzyl-1H-pyrazol-5-yl)-2-methylpropanoate (aa14-3)

A mixture of aa14-2 (700 mg, 2.71 mmol, 1 eq) and LiOH·H₂O (1 M, 5.42 mL, 2 eq) in THF (5.4 mL) was stirred at 50° C. for 16 hr. The reaction was concentrated in vacuum to remove the THF. The reaction was diluted with water (5 mL) and MTBE (5 mL). The aqueous layer was separated and adjusted to pH=6 with 1N HCl. The organic layer was discarded. The aqueous layer was extracted with EtOAc (5 mL*3). The organic layers were combined and washed with brine (5 mL), dried over anhydrous Na₂SO₄, filtered and concentrated in vacuum to give aa14-3 (600 mg, 2.46 mmol, 90.64% yield) as a white solid.

¹H NMR (400 MHz, CHLOROFORM-d) δ=7.53 (s, 1H), 7.26-7.17 (m, 3H), 6.97 (d, J=7.4 Hz, 2H), 6.24 (s, 1H), 5.23 (s, 2H), 1.54 (s, 6H).

### Step 3: Synthesis of 2-methyl-2-(1H-pyrazol-5-yl)propanoic acid (aa14)

A mixture of aa14-3 (500 mg, 2.05 mmol, 1 eq), TFA (23.34 mg, 204.68 μmol, 15.15 μL, 0.1 eq) and 10% Pd/C (100 mg) in EtOH (5 mL) was stirred at 80° C. under 100 Psi of H₂ for 16 hr. The reaction was filtered through a pad of Celite, the cake was washed with EtOH (5 mL*3). The filtrate was concentrated in vacuum to give aa14 (300 mg, 1.52 mmol, 74.27% yield, 78.11% purity) as a light yellow oil. LCMS (ESI): RT=1.036 min, m/z calcd. for C₇H₁₁N₂O₂ [M+H]⁺155.07, found 155.0. Reverse phase LC-MS was carried out using method A.

¹H NMR (400 MHz, METHANOL-d4) δ=7.57-7.50 (m, 1H), 6.26 (d, J=1.8 Hz, 1H), 1.56 (s, 6H).

### 2.6 the Synthesis of Unnatural Amino Acid [Aa20: (R)-Isomer and Aa21: (S)-Isomer]

Scheme 7 outlines the synthesis of unnatural amino acids (aa20) and (aa21):

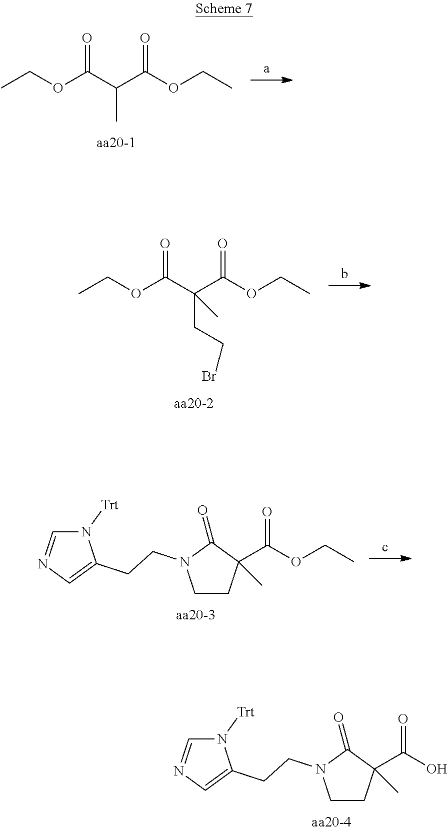

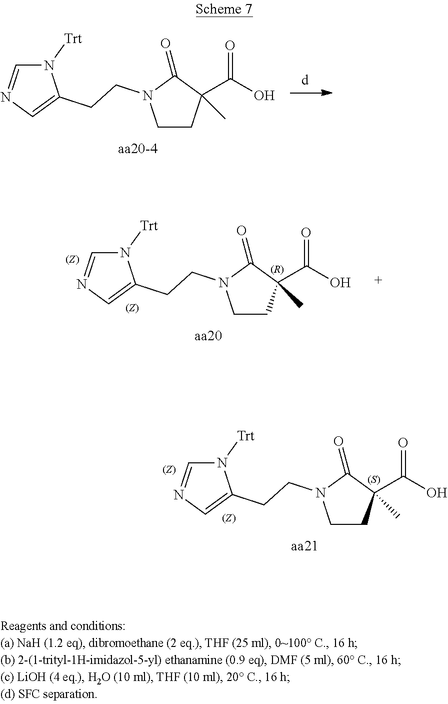

### Step 1: Synthesis of diethyl 2-(2-bromoethyl)-2-methylmalonate (aa20-2)

A volume of THF (10 mL) was added to NaH (252.57 mg, 6.31 mmol, 60% purity, 1.1 eq.) under a N₂ atmosphere. The THF solution was cooled to 0° C. Compound aa20-1 (1 g, 5.74 mmol, 980.39 μL, 1 eq.) in THF (5 mL) was added over 30 min with stirring. The reaction mixture was allowed to stir for 60 min at 20° C. The generated enolate was dripped into a 1,2-dibromoethane (2.16 g, 11.48 mmol, 866.23 μL, 2 eq.) in THF (10 mL) over 60 min with stirring under nitrogen atmosphere. The reaction mixture was then heated to 100° C. in solvent for 14 hr. TLC indicated that the reactant was consumed, and one major new spot with lower polarity was detected. The residue was poured into 1N HCl to adjust pH 2-4. Then aqueous phase was extracted with ethyl acetate (30 mL*3). The combined organic phase was washed with brine (50 mL), dried over anhydrous Na₂SO₄, filtered and concentrated in vacuum. The residue was purified by flash silica gel chromatography (ISCO@; 24 g SepaFlash@ Silica Flash Column, Eluent of 0˜20% Ethyl acetate/Petroleum ether gradient @ 20 mL/min) for 40 min with 0.8 L solvent. The compound aa20-2 (1.2 g, 4.27 mmol, 74.35% yield) was obtained as a pale yellow liquid.

¹H NMR (400 MHz, CHLOROFORM-d) δ 4.18 (q, J=7.20 Hz, 4H), 3.33-3.41 (m, 2H), 2.39-2.47 (m, 2H), 1.43 (s, 3H), 1.22 (s, 6H)

### Step 2: Synthesis of ethyl 3-methyl-2-oxo-1-(2-(1-trityl-1H-imidazol-5-yl)ethyl)pyrrolidine-3-carboxylate (aa20-3)

To a solution of 2-(1-trityl-1H-imidazol-5-yl) ethanamine (1 g, 2.83 mmol, 1 eq.) in DMF (10 mL) was added aa20-2 (874.95 mg, 3.11 mmol, 1.1 eq.). The mixture was stirred at 60° C. for 16 hr. LCMS showed the material was consumed completely and the desired product was observed as the major. The residue was poured into water (100 mL). The aqueous phase was extracted with ethyl acetate (50 mL*3). The combined organic phase was washed with brine (100 mL), dried over anhydrous Na₂SO₄, filtered and concentrated in vacuum. The crude product was purified by C-18 reverse phase chromatography (ISCO@; 80 g, C-18 Column, Eluent of 0˜100% acetonitrile/H₂O gradient @ 40 mL/min, 60 min with total volume 2400 mL) to provide a yellow solid. The compound aa20-3 (650 mg, 903.47 μmol, 31.93% yield, 70.55% purity) was obtained as a yellow solid.

LCMS (ESI): RT=0.861 min, m/z calcd. for C₃₂H₃₄N₃O₃508.25 [M+H]⁺, found 508.3. Reverse phase LC-MS was carried out using method A.

### Step 3: Synthesis of 3-methyl-2-oxo-1-(2-(1-trityl-1H-imidazol-5-yl)ethyl)pyrrolidine-3-carboxylic acid (aa20-4)

To a mixture of aa20-3 (650 mg, 1.28 mmol, 1 eq.) was added LiOH·H₂O (268.67 mg, 6.40 mmol, 5 eq.) in H₂O (10 mL) and THF (10 mL). The mixture was stirred at 20° C. for 16 hr. LCMS showed the material was consumed completely and the desired product was observed as the major. The residue was poured into H₂O (50 mL), added 5% KHSO₄ to adjust pH 2˜3. The aqueous phase was extracted with ethyl acetate (50 mL*3). The combined organic phase was washed with brine (50 ml), dried with anhydrous Na₂SO₄, filtered and concentrated in vacuum. The crude was purified by prep-HPLC (column: Phenomenex Synergi Max-RP 250*50 mm*10 um; mobile phase: water (0.225% FA)-ACN; B %: 30%-60%, 60 min) to give aa20-4 (180 mg, 375.34 μmol, 29.31% yield) as a white solid.

LCMS (ESI): RT=2.391 min, m/z calcd. for C₃₀H₃₀N₃03480.6, found 480.3 [M+H]⁺. Reverse phase LC-MS was carried out using a Merck RP-18e 25-2 mm column, with a flow rate of 1.5 mL/min, eluting with a gradient of 10% to 80% acetonitrile containing 0.02% TFA (solvent B) and water containing 0.04% TFA (solvent A)

¹H NMR (ES8586-496-P1B1) 1H NMR (400 MHz, METHANOL-d4) δ 7.92 (s, 1H), 7.35-7.42 (m, 9H), 7.12-7.19 (m, 6H), 6.97 (s, 1H), 3.39-3.64 (m, 3H), 3.32 (br d, J=3.75 Hz, 1H), 2.84(br t, J=5.95 Hz, 2H), 2.34-2.42 (m, 1H), 1.86 (td, J=7.99, 12.90 Hz, 1H), 1.25 (s, 3H)

### Step 4: Synthesis of (R)-3-methyl-2-oxo-1-(2-(1-trityl-1H-imidazol-5-yl)ethyl)pyrrolidine-3-carboxylic acid (aa20) and (S)-3-methyl-2-oxo-1-(2-(1-trityl-1H-imidazol-5-yl)ethyl)pyrrolidine-3-carboxylic acid (aa21)

The absolute configurations of aa20 (R) and aa21 (S) were not confirmed.

Compound aa20-4 (180 mg, 375.34 μmol) was purified by prep-SFC (column: DAICEL CHIRALPAK IC (250 mm*30 mm, 10 μm; mobile phase: 0.1% NH₃H₂O MEOH; B %: 50%-50%, 80 min). Isomer (R) aa20 (86 mg, 177.18 μmol, 94.41% yield, 98.8% purity) was obtained as a white solid, and isomer (S) aa21 (85 mg, 175.29 μmol, 93.41% yield, 98.9% purity) was also obtained as a white solid.

Isomer aa20 on SFC-HPLC: RT=2.346 min. HPLC conditions: Chiralpak IC-3 100×4.6 mm I.D., 3 μm, flow rate 2.8 mL/min. eluting with a gradient of 40% methanol (0.05% DEA) (solvent B) and CO₂ (solvent A).

Isomer aa21 on SFC-HPLC: RT=3.607 min. HPLC conditions: Chiralpak IC-3 100×4.6 mm I.D., 3 μm, flow rate 2.8 mL/min. eluting with a gradient of 40% methanol (0.05% DEA) (solvent B) and CO₂ (solvent A).

### 2.7 the Synthesis of Unnatural Amino Acid [Aa22 (S)-Isomer and Aa23 (R)-Isomer]

Scheme 8 outlines the synthesis of unnatural amino acids (aa22) and (aa23):

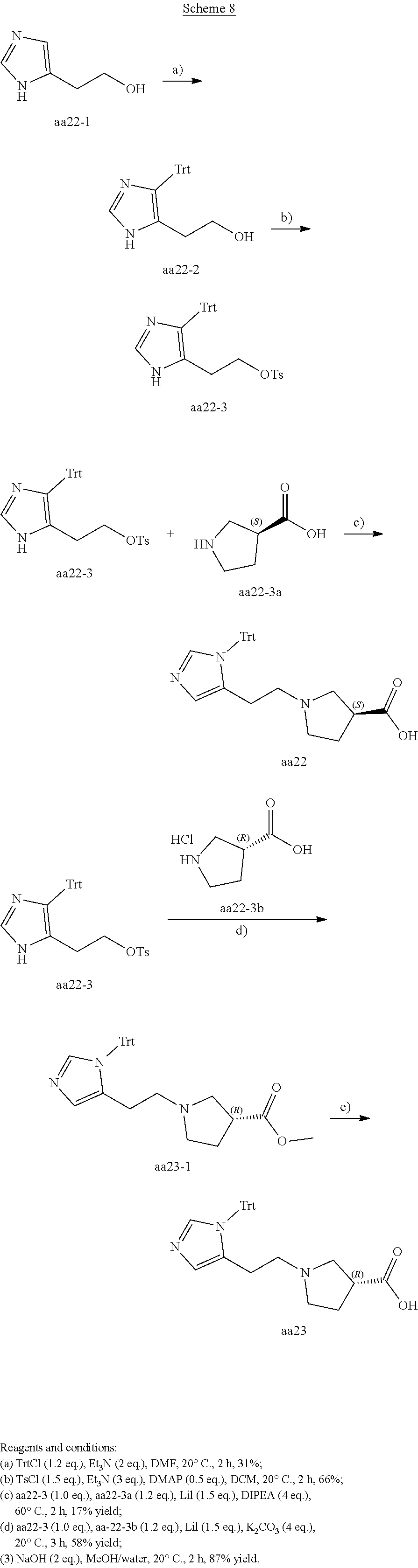

### Step 1: Synthesis of 2-(1-trityl-1H-imidazol-4-yl) ethanol (aa22-2)

To a solution of aa22-1 (2 g, 17.84 mmol, 1 eq) in DMF (40 mL) were added TEA (3.61 g, 35.67 mmol, 4.97 mL, 2 eq), [chloro(diphenyl)methyl]benzene (5.97 g, 21.40 mmol, 1.2 eq) at 20° C. The reaction mixture was stirred for 2 hr at 20° C. The reaction progress was monitored by TLC (DCM:MeOH=10:1), which indicated that the starting material was consumed and one new spot was observed. The reaction mixture was quenched by NaHCO₃ (sat. aq., 20 mL) and extracted with EtOAc (50 mL*2). The combined organic layers were washed with brine (20 mL), dried over Na₂SO₄, filtered and concentrated under reduced pressure to give a residue. The residue was purified by flash silica gel chromatography (ISCO@; 20 g SepaFlash@ Silica Flash Column, Eluent of 0˜100% Ethyl acetate/Petroleum ether then 0˜10% MeOH (0.5% TEA additive)/DCM gradient @ 30 mL/min) for 25 min with total volume 1.6 L. Compound aa22-2 (2.2 g, 5.59 mmol, 31.32% yield, 90% purity) was obtained as a white solid.

LCMS: (ESI): RT=1.397 min, m/z calcd. for C₂₄H₂₃N₂O 355.2, found 355.2 [M+H]⁺; Reverse phase LC-MS was carried out using method B.

¹H NMR (400 MHz, CHLOROFORM-d) δ=7.39-7.31 (m, 10H), 7.17-7.11 (m, 6H), 6.63-6.57 (m, 1H), 3.89 (t, J=5.6 Hz, 2H), 3.64 (br s, 1H), 2.76 (t, J=5.5 Hz, 2H).

### Step 2: Synthesis of 2-(1-trityl-1H-imidazol-4-yl)ethyl 4-methylbenzenesulfonate (aa22-3)

To a solution of aa22-2 (2.2 g, 5.59 mmol, 1 eq) in DCM (20 mL) were added TEA (1.70 g, 16.76 mmol, 2.33 mL, 3 eq), 4-methylbenzenesulfonyl chloride (1.60 g, 8.38 mmol, 1.5 eq) and DMAP (341.23 mg, 2.79 mmol, 0.5 eq) at 20° C. The reaction mixture was stirred for 2 hr at 20° C. The reaction progress was monitored by LC-MS which indicated no starting material remained and formation of desired product. The reaction mixture was quenched by NaHCO₃ (sat. aq., 50 mL) and extracted with EtOAc (50 mL*2). The combined organic layers were washed with brine (20 mL×2), dried over Na₂SO₄, filtered and concentrated under reduced pressure to give a residue. The residue was purified by flash silica gel chromatography (ISCO@; 24 g SepaFlash@ Silica Flash Column, Eluent of 0˜5% MeOH (4% TEA additive)/DCM gradient @ 20 mL/min) for 35 min with total volume 1.2 L. Compound aa22-3 (2.1 g, 3.72 mmol, 66.52% yield, 90% purity) was obtained as a brown solid. LCMS: (ESI): RT=0.775 min, m/z calcd. for C₃₁H₂₃N₂O₃SNa 531.2 [M+H]⁺, found 531.1. Reverse phase LC-MS was carried out using method D.

¹H NMR (400 MHz, CHLOROFORM-d) δ=7.73 (d, J=8.3 Hz, 2H), 7.38-7.28 (m, 12H), 7.15-7.08 (m, 6H), 6.61 (s, 1H), 4.27 (t, J=7.0 Hz, 2H), 2.91-2.86 (m, 2H), 2.42 (s, 3H).

### Step 3: Synthesis of (R)-1-(2-(1-trityl-1H-imidazol-4-yl)ethyl)pyrrolidine-3-carboxylic acid (aa22)

To a solution of aa22-3 (500 mg, 983.03 μmol, 1 eq.) in DMF (2 mL) were added aa22-3a (135.81 mg, 1.18 mmol, 1.2 eq.), LiI (197.36 mg, 1.47 mmol, 56.55 μL, 1.5 eq.), and DIPEA (508.20 mg, 3.93 mmol, 684.91 μL, 4.0 eq.). Then the mixture was heated to 60° C. and stirred at 60° C. for 2 hr. LCMS showed that reactant was consumed completely and the desired MS was detected. The reaction mixture was quenched by addition of water (10 mL), and then diluted with EtOAc (20 mL), then extracted with EtOAc (20 mL*2). The aqueous layers were acidfied with 2M HCl to pH 6. Then the aqueous phase was extracted with DCM (20 mL*5). The combined organic layers were concentrated to give the residue. The crude product was purified by reversed-phase HPLC (neutral condition). Product aa22 (80 mg, 168.31 μmol, 17.12% yield, 95% purity) was obtained as an off-white solid. LCMS (ESI): RT=1.986 min mass calcd. for C₂₉H₃₀N₃O₂ 452.23, m/z found 452.3 [M+H]⁺. Reverse phase LC-MS was carried out using method A.

### Step 4: Synthesis of methyl (3S)-1-[2-(1-tritylimidazol-4-yl)ethyl]pyrrolidine-3-carboxylate (aa23-1)

To a solution of aa22-3 (750 mg, 1.47 mmol, 1 eq.) in DMF (5 mL) was added K₂CO₃ (815.17 mg, 5.90 mmol, 4 eq.), aa22-3b (293.05 mg, 1.77 mmol, 1.2 eq., HCl) and LiI (296.04 mg, 2.21 mmol, 84.83 μL, 1.5 eq.) at 20° C. The reaction mixture was stirred for 3 hr at 20° C. The reaction progress was monitored by LC-MS, which indicated no starting material remained and formation of desired product. The mixture was cooled to 25° C. and poured into water (30 mL) and stirred for 5 min. The aqueous phase was extracted with ethyl acetate (50 mL*2). The combined organic phase was washed with brine (15 mL*2), dried over anhydrous Na₂SO₄, filtered and concentrated in vacuum. The residue was purified by flash silica gel chromatography (ISCO@; 20 g SepaFlash@ Silica Flash Column, Eluent of 0˜100% Ethyl acetate/Petroleum ether then 0˜10% MeOH (0.5% TEA additive)/DCM gradient @ 30 mL/min) for 25 min with total volume 1.6 L. Product aa23-1 (420 mg, 856.99 μmol, 58.12% yield, 95% purity) was obtained as a brown oil.

LCMS: (ESI): RT=0.717 min, m/z calcd. for C₃₀H₃₁N₃O₂ 466.2 [M+H]⁺, found 465.9; LC-MS Conditions: Reverse phase LC-MS was carried out using method D.

¹H NMR (400 MHz, CHLOROFORM-d) δ=7.40-7.30 (m, 10H), 7.20-7.09 (m, 6H), 6.58 (s, 1H), 3.75-3.64 (m, 3H), 3.10-2.95 (m, 2H), 2.83-2.74 (m, 5H), 2.72-2.64 (m, 1H), 2.55 (q, J=8.0 Hz, 1H), 2.14-2.07 (m, 2H).

### Step 5: Synthesis of methyl (3S)-1-[2-(1-tritylimidazol-4-yl)ethyl]pyrrolidine-3-carboxylate (aa23)

To a solution of aa23-1 (390.00 mg, 837.66 μmol, 1 eq.) in MeOH (3 mL) and Water (3 mL) was added NaOH (67.01 mg, 1.68 mmol, 2 eq.) at 20° C. The reaction mixture was stirred for 2 hr at 20° C. The reaction progress was monitored by LCMS which indicated no starting material remained and formation of desired product. After completion, the reaction mixture was cooled to room temperature. The reaction mixture was concentrated under reduced pressure to remove solvent. The residue was adjusted to pH=6 with 1M HCl and dried by lyophilization. The residue was triturated with DCM/MeOH=10/1 (15 mL*3). The combined organic phase was dried over anhydrous Na₂SO₄, filtered and concentrated in vacuum. Product aa23 (350 mg, 736.34 μmol, 87.90% yield, 95% purity) was obtained as a brown solid.

LCMS: (ESI): RT=0.708 min, m/z calcd. for C₂₉H₃₀N₃O₂ 452.2 [M+H]⁺, found 451.9; Reverse phase LC-MS was carried out using method D.

¹H NMR (400 MHz, CHLOROFORM-d) δ=7.38 (s, 1H), 7.36-7.31 (m, 9H), 7.12 (dd, J=3.3, 6.3 Hz, 6H), 6.64 (s, 1H), 3.83 (br s, 1H), 3.56 (br s, 1H), 3.20 (br d, J=18.5 Hz, 2H), 3.05-2.96 (m, 4H), 2.65-2.61 (m, 1H), 2.42-2.30 (m, 1H), 2.18-2.09 (m, 1H).

### 2.8 the Synthesis of Unnatural Amino Acid (Aa27)

Scheme 9 outlines the synthesis of unnatural amino acid (aa27):

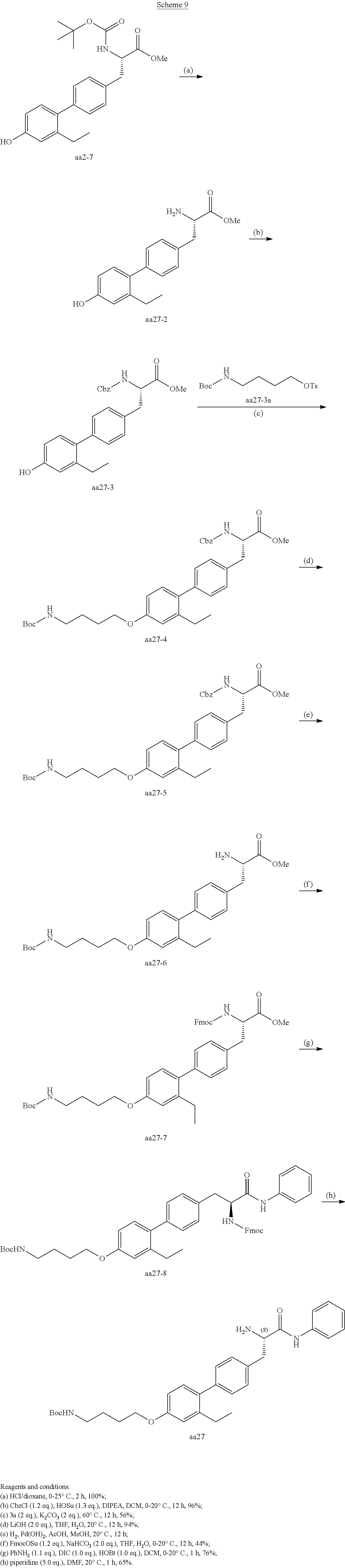

### Step 1: Synthesis of methyl (2S)-2-amino-3-[4-(2-ethyl-4-hydroxy-phenyl)phenyl]propanoate (aa27-2)

Compound aa27-1 (3.5 g, 8.76 mmol, 1 eq.) was dissolved in HCl/dioxane (4 M, 100 mL, 45.65 eq.) at 0° C. The mixture was stirred at 25° C. for 2 hr. The reaction progress was monitored by LCMS. The reaction mixture was concentrated under reduced pressure to give the crude product. Compound aa27-2 (2.6 g, crude) was obtained as a colorless oil.

LCMS (ESI): RT=0.772 min, mass calcd. for C₁₈H₂₂NO₃, 300.16 [M+H]⁺, m/z found 300.00 [M+H]⁺. Reverse phase LC-MS was carried out using method A.

### Step 2: Synthesis of methyl (2S)-2-(benzyloxycarbonylamino)-3-[4-(2-ethyl-4-hydroxy-phenyl) phenyl]propanoate (aa27-3)

To a solution of HOSu (1.25 g, 10.86 mmol, 1.3 eq.) in DCM (50 mL) was added DIPEA (3.24 g, 25.05 mmol, 4.36 mL, 3 eq.) and CbzCl (1.57 g, 9.19 mmol, 1.31 mL, 1.1 eq.) at 0° C. After stirred at 25° C. for 2 hr, then Compound aa27-2 (2.5 g, 8.35 mmol, 1 eq.) was added and the mixture was stirred at 20° C. for 10 hr. The reaction progress was monitored by LC-MS and TLC. The mixture was diluted with DCM (30 mL) and washed with H₂O (30 mL*2). The organic layer was dried over Na₂SO₄, filtered and concentrated to give a residue, then purified by column chromatography (SiO₂, Petroleum ether/Ethyl acetate=2/1). Compound aa27-3 (3.5 g, 8.07 mmol, 96.68% yield) was obtained as a yellow oil.

LCMS (ESI): RT=0.992 min, mass calcd. for C26H27NNaO5+, 456.18 [M+Na]+, m/z found 456.2 [M+Na]⁺. Reverse phase LC-MS was carried out using method A.

¹H NMR (400 MHz, DMSO-d6) δ ppm 9.35 (s, 1H) 7.88 (br d, J=8.07 Hz, 1H) 7.21-7.38 (m, 7H) 7.15 (br d, J=8.07 Hz, 2H) 6.93 (d, J=8.31 Hz, 1H) 6.70 (d, J=2.20 Hz, 1H) 6.63 (dd, J=8.07, 2.45 Hz, 1H) 4.99 (br s, 2H) 4.24-4.36 (m, 1H) 3.60-3.65 (m, 3H) 3.07 (br dd, J=13.69, 4.89 Hz, 1H) 2.91 (br dd, J=13.57, 10.39 Hz, 1H) 2.40-2.48 (m, 2H) 0.99 (t, J=7.58 Hz, 3H).

### Step 3: Synthesis of (S)-methyl 2-(((benzyloxy)carbonyl)amino)-3-(4′-(4-((tert-butoxycarbonyl) amino)butoxy)-2′-ethyl-[1,1′-biphenyl]-4-yl)propanoate (aa27-4)

To a solution of aa27-3 (3.3 g, 7.61 mmol, 1 eq.) and aa27-3a (5.23 g, 15.23 mmol, 2 eq.) in DMF (30 mL) was added K₂CO₃ (2.10 g, 15.23 mmol, 2 eq.). The mixture was stirred at 60° C. for 12 hr. The reaction progress was monitored by LC-MS and TLC. The reaction mixture was diluted with EtOAc (100 mL) and washed with H₂O (80 mL*3). The organic layer was dried over Na₂SO₄, filtered and concentrated to give a residue, then purified by column chromatography (SiO₂, Petroleum ether/Ethyl acetate=10/1 to 2/1). Compound aa27-4 (2.9 g, 4.32 mmol, 56.70% yield, 90% purity) was obtained as a yellow oil.

LCMS (ESI): RT=1.140 min, mass calcd. for C₃₅H₄₄N₂NaO₇, 627.30 [M+Na]⁺, m/z found 627.2 [M+Na]⁺. Reverse phase LC-MS was carried out using method A.

### Step 4: Synthesis of (S)-2-(((benzyloxy)carbonyl)amino)-3-(4′-(4-((tert-butoxycarbonyl) amino) butoxy)-2′-ethyl-[1,1′-biphenyl]-4-yl)propanoic acid (aa27-5)

To a solution of aa27-4 (2.7 g, 4.46 mmol, 1 eq.) in THF (30 mL) were added a solution of LiOH·H₂O (374.72 mg, 8.93 mmol, 2 eq.) in H₂O (15 mL). The mixture was stirred at 20° C. for 2 hr. The reaction progress was monitored by LC-MS. The mixture was adjusted to pH 3-4 with 1M HCl and extracted with EtOAc (20 mL*3). The organic layer was dried over Na₂SO₄, filtered and concentrated to give the product. Compound aa27-5 (2.5 g, 4.23 mmol, 94.79% yield) was obtained as a yellow oil.

LCMS (ESI): RT=1.082 min, mass calcd. for C₃₄H₄₂N₂NaO₇, 613.29, m/z found 613.2 [M+Na]⁺. Reverse phase LC-MS was carried out using method A.

¹H NMR (400 MHz, CHLOROFORM-d) δ ppm 7.29-7.36 (m, 5H) 7.14-7.21 (m, 4H) 7.07 (br d, J=8.56 Hz, 1H) 6.81 (s, 1H) 6.73 (br d, J=7.83 Hz, 1H) 5.27 (br d, J=7.58 Hz, 1H) 5.05-5.16 (m, 2H) 4.68-4.75 (m, 1H) 4.50-4.68 (m, 2H) 3.99 (br s, 2H) 3.20-3.28 (m, 2H) 2.53 (q, J=7.42 Hz, 2H) 1.62-1.68 (m, 2H) 1.51-1.57 (m, 2H) 1.44 (br s, 9H) 1.07 (t, J=7.46 Hz, 3H).

### Step 5: Synthesis of (2S)-2-amino-3-[4-[4-[4-(tert-butoxycarbonylamino)butoxy]-2-ethyl-phenyl]phenyl]propanoic acid (aa27-6)

To a solution of aa27-5 (1.5 g, 2.54 mmol, 1 eq.) in MeOH (30 mL) were added Pd(OH)₂/C (300 mg, 213.62 μmol, 10% purity) and AcOH (105.00 mg, 1.75 mmol, 0.1 mL). The mixture was stirred under H₂ at 20° C. for 12 hr. The reaction progress was monitored by LC-MS. The mixture was filtered and concentrated to give the crude product. Compound aa27-6 (1.16 g, crude) was obtained as a yellow oil.

LCMS (ESI): RT=0.890 min, mass calcd. for C₂₆H₃₆N₂NaO₅, 479.25, m/z found 479.1 [M+Na]⁺. Reverse phase LC-MS was carried out using method A.

### Step 6: Synthesis of (S)-2-((((9H-fluoren-9-yl)methoxy)carbonyl)amino)-3-(4′-(4-((tert-butoxycarbonyl)amino)butoxy)-2′-ethyl-[1,1′-bipheny]-4-yl)propanoic acid (aa27-7)

To a solution of aa27-6 (1.16 g, 2.54 mmol, 1 eq.) in THF (15 mL) and H₂O (8 mL) were added NaHCO₃ (426.64 mg, 5.08 mmol, 197.52 μL, 2 eq.) and Fmoc-OSu (1.03 g, 3.05 mmol, 1.2 eq.) at 0° C. The mixture was stirred 20° C. for 12 hr. The reaction progress was monitored by LC-MS. The mixture was adjusted to pH 3˜4 and extracted with EtOAc (20 mL*2). The organic layer was dried over Na₂SO₄, filtered and concentrated to give a residue, then purified by prep-HPLC (AcOH condition; MeCN/H₂O, 0˜100%). Compound aa27-7 (950 mg, 1.12 mmol, 44.09% yield, 80% purity) was obtained as a colorless oil.

LCMS (ESI): RT=1.142 min, mass calcd. for C₄₁H₄₆N₂NaO₇, 701.32, m/z found 701.0 [M+Na]⁺. Reverse phase LC-MS was carried out using method A.

HPLC: RT=4.33 min, Mobile Phase: 2.75 ML/4 L TFA in water (solvent A) and 2.5 ML/4 L TFA in acetonitrile (solvent B), using the elution gradient 10%-80% (solvent B) over 6 minutes and holding at 80% for 2 minutes at a flow rate of 1.2 ml/min; Column: Ultimate C18 3.0*50 mm, 3 μm.

SFC: RT=0.598 min, Column: Chiralpak AD-3 50×4.6 mm I.D., 3 μm; Mobile phase: A: C02 B: ethanol (0.05% DEA) Isocratic: 40% B; Flow rate: 4 mL/min.

¹H NMR (400 MHz, CHLOROFORM-d) δ ppm 7.29-7.36 (m, 5H) 7.14-7.21 (m, 4H) 7.07 (br d, J=8.56 Hz, 1H) 6.81 (s, 1H) 6.73 (br d, J=7.83 Hz, 1H) 5.27 (br d, J=7.58 Hz, 1H) 5.05-5.16 (m, 2H) 4.68-4.75 (m, 1H) 4.50-4.68 (m, 2H) 3.99 (br s, 2H) 3.20-3.28 (m, 2H) 2.53 (q, J=7.42 Hz, 2H) 1.62-1.68 (m, 2H) 1.51-1.57 (m, 2H) 1.44 (br s, 9H) 1.07 (t, J=7.46 Hz, 3H).

### Step 7: Synthesis of 9H-fluoren-9-ylmethyl N-[(1S)-2-anilino-1-[[4-[4-[4-(tert-butoxycarbonylamino)butoxy]-2-ethyl-phenyl]phenyl]methyl]-2-oxo-ethyl]carbamate (aa27-8)

To a solution of aa27-7 (250 mg, 368.29 μmol, 1 eq.) and HOBt (49.76 mg, 368.29 μmol, 1 eq.) in DCM (1.5 mL) were added aniline (36.01 mg, 386.71 μmol, 35.31 μL, 1.05 eq.) and a solution of DIC (46.48 mg, 368.29 μmol, 57.03 μL, 1 eq.) in DCM (0.5 mL) at 0° C. The mixture was stirred at 20° C. for 1 hr. The reaction progress was monitored by LC-MS. The mixture was diluted with DCM (20 mL) and washed with H₂O (10 mL*2). The organic layer was dried over Na₂SO₄, filtered and concentrated to give a residue. The residue was purified by flash silica gel chromatography (ISCO@; 12 g SepaFlash@ Silica Flash Column, Eluent of 0˜50% Ethyl acetate/Petroleum ether gradient @ 20 mL/min). Compound aa27-8 (220 mg, 283.05 μmol, 76.86% yield, 97% purity) was obtained as a white solid.

LCMS (ESI): RT=1.198 min, mass calcd. for C₄₇H₅₁N₃NaO₆ 776.37, m/z found 777.3 [M+Na]⁺. Reverse phase LC-MS was carried out using method A.

HPLC: RT=8.07 min, Reverse phase HPLC analysis was carried out using method D.SFC: RT=2.186 min; Column: Chiralcel OD-3 50×4.6 mm I.D., 3 μm; Mobile phase: A: C02 B: ethanol (0.05% DEA); Isocratic: 40% B; Flow rate: 4 mL/min.

¹H NMR (400 MHz, CHLOROFORM-d) δ ppm 7.74 (br d, J=7.58 Hz, 2H) 7.51-7.58 (m, 3H) 7.30-7.40 (m, 4H) 7.28 (br d, J=7.58 Hz, 4H) 7.17-7.24 (m, 4H) 7.07-7.11 (m, 1H) 7.02 (d, J=8.31 Hz, 1H) 6.81 (d, J=2.45 Hz, 1H) 6.72 (dd, J=8.31, 2.45 Hz, 1H) 5.57 (br s, 1H) 4.32-4.69 (m, 4H) 4.20 (t, J=6.72 Hz, 1H) 3.99 (t, J=6.24 Hz, 2H) 3.06-3.29 (m, 4H) 2.49 (q, J=7.42 Hz, 2H) 1.77-1.87 (m, 2H) 1.68 (dt, J=14.55, 7.15 Hz, 2H) 1.44 (s, 9H) 1.03 (t, J=7.58 Hz, 3H).

### Step 8: Synthesis of tert-butyl N-[4-[4-[4-[(2S)-2-amino-3-anilino-3-oxo-propyl]phenyl]-3-ethyl-phenoxy]butyl]carbamate (aa27)

To a solution of aa27-8 (220 mg, 291.81 μmol, 1 eq.) in DMF (2 mL) was added a solution of piperidine (124.24 mg, 1.46 mmol, 5 eq.). The mixture was stirred at 20° C. for 1 hr. The reaction progress was monitored by LC-MS. The mixture was diluted with EtOAc (30 mL) and washed with H₂O (10 mL*3). The organic layer was dried over Na₂SO₄, filtered and concentrated to give a residue, then purified by column chromatography (SiO₂, EtOAc:MeOH=1:0 to 5:1). Compound aa27 (105 mg, 191.56 μmol, 65.65% yield, 97% purity) was obtained as a yellow foam.

LCMS (ESI): RT=0.953 min, mass calcd. for C₃₂H₄₁N₃NaO₄ 554.30, m/z found 554.1 [M+Na]⁺. Reverse phase LC-MS was carried out using method A. HPLC: RT=9.13 min, Reverse phase HPLC analysis was carried out using method C.

SFC: RT=3.185 min, Column: Chiralpak AS-3 100×4.6 mm I.D., 3 μm; Mobile phase: A: CO₂ B:ethanol (0.1% ethanolamine) Gradient: from 5% to 40% of B in 4.5 min and hold 40% for 2.5 min, then 5% of B for 1 min; Flow rate: 2.8 mL/min.

¹H NMR (400 MHz, CHLOROFORM-d) δ ppm 9.45 (s, 1H) 7.60 (br d, J=7.83 Hz, 2H) 7.33 (t, J=7.83 Hz, 2H) 7.20-7.29 (m, 4H) 7.06-7.13 (m, 2H) 6.84 (d, J=2.20 Hz, 1H) 6.75 (dd, J=8.31, 2.45 Hz, 1H) 4.68 (br s, 1H) 4.01 (t, J=6.11 Hz, 2H) 3.82 (br d, J=5.38 Hz, 1H) 3.41 (br dd, J=13.82, 3.30 Hz, 1H) 3.13-3.27 (m, 2H) 2.84 (br dd, J=13.82, 9.90 Hz, 1H) 2.56 (q, J=7.50 Hz, 2H) 1.79-1.86 (m, 2H) 1.69 (quin, J=7.15 Hz, 2H) 1.45 (s, 9H) 1.09 (t, J=7.58 Hz, 3H)

### 2.9 the Synthesis of Unnatural Amino Acid (Aa28)

Scheme 10 outlines the synthesis of unnatural amino acid (aa28):

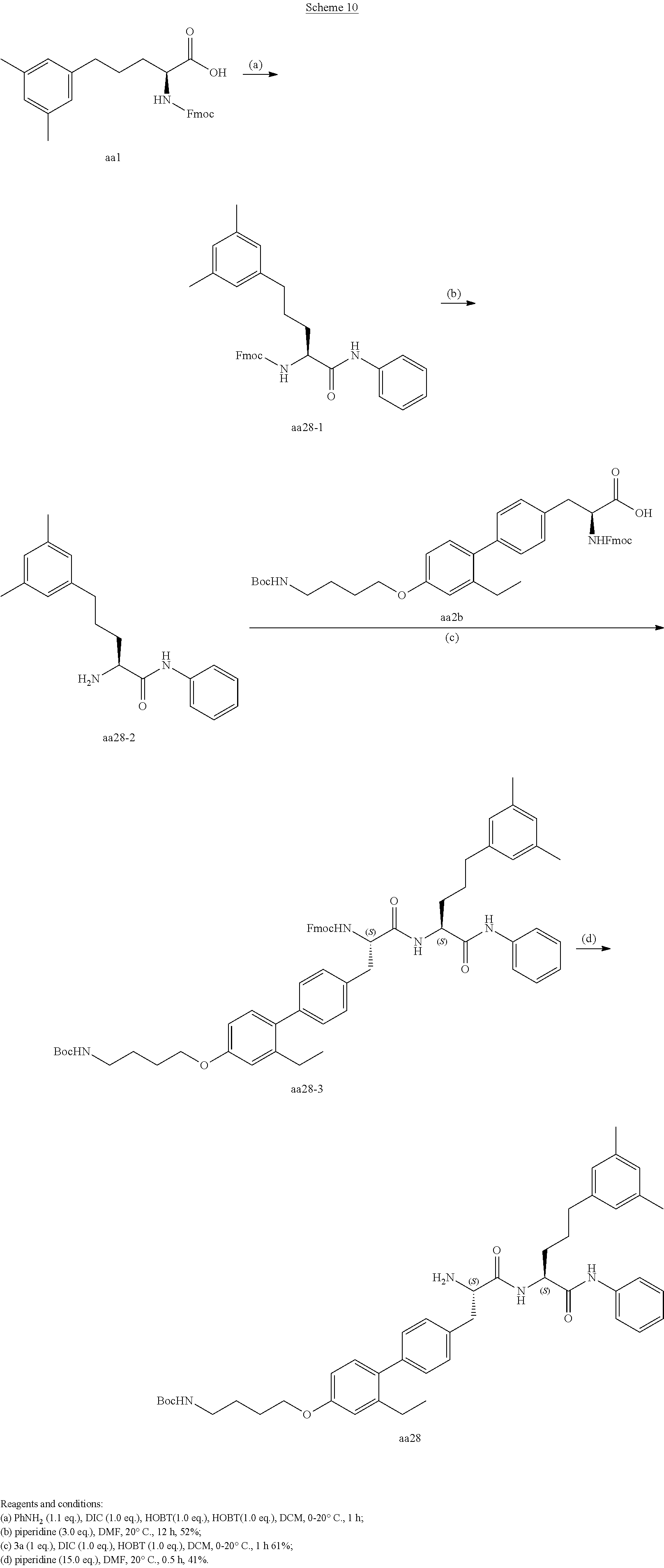

### Step 1: Synthesis of 9H-fluoren-9-ylmethyl N-[(1S)-4-(3,5-dimethylphenyl)-1-(phenylcarbamoyl) butyl]carbamate (aa28-1)

To a solution of aa1 (300 mg, 676.39 μmol, 1 eq.) and HOBt (91.40 mg, 676.39 μmol, 1 eq.) in DCM (1.5 mL) were added aniline (66.14 mg, 710.21 μmol, 64.84 μL, 1.05 eq.) at 0° C. A solution of DIC (85.36 mg, 676.39 μmol, 1 eq.) in DCM (0.5 mL) was added to the mixture. The reaction was stirred at 20° C. for 1 hr. The reaction progress was monitored by LC-MS. The mixture was diluted with DCM (20 mL) and washed with H₂O (10 mL*2). The organic layer was dried over Na₂SO₄, filtered and concentrated to give the crude product. Compound aa28-1 (400 mg, crude) was obtained as a yellow solid.

LCMS (ESI): RT=1.178 min, mass calcd. for C₃₄H₃₅N₂O₃+519.26 [M+H]⁺, m/z found 519.1 [M+H]⁺. Reverse phase LC-MS was carried out using method A.

### Step 2: Synthesis of (2S)-2-amino-5-(3,5-dimethylphenyl)-N-phenyl-pentanamide (aa28-2)

To a solution of aa28-1 (400 mg, 771.24 μmol, 1 eq.) in DMF (2 mL) were added piperidine (197.01 mg, 2.31 mmol, 3 eq) at 20° C. The reaction was stirred at 20° C. for 12 hr. The reaction progress was monitored by LC-MS and TLC. The mixture was diluted with EtOAc (30 mL) and washed with H₂O (10 mL*3). The organic layer was dried over Na₂SO₄, filtered and concentrated to give a residue, then purified by flash silica gel chromatography (ISCO@; 12 g SepaFlash@ Silica Flash Column, Eluent of 0˜100% Ethyl acetate/Petroleum ethergradient @ 20 mL/min). Compound aa28-2 (150 mg, 399.79 μmol, 51.84% yield, 79% purity) was obtained as a yellow foam. LCMS (ESI): RT=0.861 min, mass calcd. for C₁₉H₂₅N₂O 297.2 [M+H]⁺, m/z found 297.1 [M+H]⁺. Reverse phase LC-MS was carried out using method A.

¹H NMR (400 MHz, CD3Cl) δ ppm 9.46 (br s, 1H), 7.60 (d, J=7.83 Hz, 2H), 7.33 (t, J=7.95 Hz, 2H), 7.07-7.14 (m, 1H), 6.78-6.86 (m, 4H), 3.51 (dd, J=7.95, 4.28 Hz, 1H), 2.57-2.64 (m, 2H), 2.29 (s, 6H), 1.67-1.81 (m, 4H).

### Step 3: Synthesis of 9H-fluoren-9-ylmethyl N-[(1S)-1-[[4-[4-[4-(tert-butoxycarbonylamino) butoxy]-2-ethyl-phenyl]phenyl]methyl]-2-[[(1S)-4-(3,5-dimethylphenyl)-1-(phenylcarbamoyl) butyl]amino]-2-oxo-ethyl]carbamate (aa28-3)

To a solution of aa2b (343.52 mg, 506.06 μmol, 1 eq.) and HOBt (68.38 mg, 506.06 μmol, 1 eq) in DCM (2.5 mL) were added aa28-2 (150 mg, 506.06 μmol, 1 eq.) at 0° C. A solution of DIC (63.86 mg, 506.06 μmol, 1 eq.) in DCM (0.5 mL) was added to the mixture. The reaction was stirred at 20° C. for 1 hr. The reaction progress was monitored by TLC. The mixture was diluted with DCM (20 mL) and washed with H₂O (10 mL*2). The organic layer was dried over Na₂SO₄, filtered and concentrated to give a residue. The residue was purified by flash silica gel chromatography (ISCO@; 12 g SepaFlash@ Silica Flash Column, Eluent of 0˜45% Ethylacetate/Petroleum ether gradient @ 20 mL/min). Compound aa28-3 (400 mg, 309.23 μmol, 61.11% yield, 74% purity) was obtained as a yellow solid. LCMS (ESI): RT=6.617 min, mass calcd. for C₆₀H₆₈N₄NaO₇ 979.50, m/z found 979.8 [M+Na]⁺. Reverse phase LC-MS was carried out using method B.

¹H NMR (400 MHz, CD₃Cl) δ ppm 7.72-7.80 (m, 2H), 7.49-7.57 (m, 3H), 7.35-7.41 (m, 2H), 7.20-7.33 (m, 5H), 7.08-7.20 (m, 4H), 6.96-7.08 (m, 2H), 6.76-6.84 (m, 2H), 6.68-6.75 (m, 3H), 5.32-5.47 (m, 1H), 4.64 (br s, 1H), 4.52-4.60 (m, 1H), 4.42-4.51 (m, 2H), 4.37 (br s, 1H), 4.13-4.23 (m, 1H), 4.00 (t, J=6.24 Hz, 2H), 3.05-3.27 (m, 4H), 2.44-2.61 (m, 4H), 2.20-2.25 (m, 6H), 1.82-1.89 (m, 2H), 1.62-1.75 (m, 6H), 1.46 (s, 9H), 1.01-1.09 (m, 3H).

### Step 4: Synthesis of tert-butyl N-[4-[4-[4-[(2S)-2-amino-3-[[(1S)-4-(3,5-dimethylphenyl)-1-(phenylcarbamoyl)butyl]amino]-3-oxo-propyl]phenyl]-3-ethyl-phenoxy]butyl]carbamate (aa28)

To a solution of aa28-3 (380 mg, 396.99 μmol, 1 eq.) in DMF (2.5 mL) were added piperidine (507.06 mg, 5.95 mmol, 15 eq.) at 20° C. The reaction was stirred at 20° C. for 0.5 hr. The reaction progress was monitored by LC-MS. The mixture was diluted with ACN (2 mL) and purified by prep-HPLC (HCl condition; column: Agela ASB 150*25 mm*5 μm; mobile phase: [water (0.05% HCl)-ACN]; B %: 55%-85%, 8 min). Compound aa28 (125 mg, 164.97 μmol, 41.56% yield, 97% purity) was obtained as a little yellow foam.

LCMS (ESI): RT=1.061 min, mass calcd. for C₄₅H₅₈N₄NaO₅ 757.43, m/z found 757.5 [M+Na]⁺. Reverse phase LC-MS was carried out using method A. HPLC: RT=10.66 min, Reverse phase HPLC analysis was carried out using method C. SFC: RT=2.161 min, Column: Chiralpak AD-3 50*4.6 mm I.D., 3 μm; Mobile phase: A: CO₂ B:iso-propanol (0.05% DEA); Gradient: from 5% to 40% of B in 2 min and hold 40% for 1.2 min, then 5% of B for 0.8 min; Flow rate: 4 mL/min.

¹H NMR (400 MHz, CD3OD) δ ppm 7.55 (br d, J=8.07 Hz, 2H), 7.24-7.31 (m, 4H), 7.14 (d, J=8.07 Hz, 2H), 7.05-7.11 (m, 1H), 6.95 (d, J=8.31 Hz, 1H), 6.81 (d, J=2.45 Hz, 1H), 6.78 (s, 3H), 6.68-6.72 (m, 1H), 4.54 (t, J=6.72 Hz, 1H), 4.20 (t, J=6.85 Hz, 1H), 4.00 (t, J=6.11 Hz, 2H), 3.25-3.30 (m, 1H), 3.12 (br t, J=6.85 Hz, 3H), 2.54-2.63 (m, 2H), 2.51 (q, J=7.50 Hz, 2H), 2.22 (s, 6H), 1.62-1.95 (m, 8H), 1.44 (s, 9H), 1.03 (t, J=7.58 Hz, 3H).

### 2.10 the Synthesis of Unnatural Amino Acid (Aa29)

Scheme 11 outlines the synthesis of unnatural amino acid (aa29):

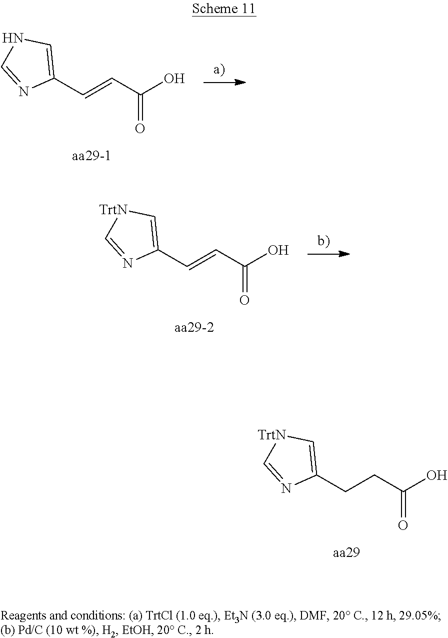

### Step 1: Synthesis of (E)-3-(1-trityl-1H-imidazol-5-yl) acrylic acid (aa29-2)

Compounds aa29-1 (2 g, 14.48 mmol, 1.0 eq.), TEA (4.40 g, 43.44 mmol, 6.05 mL, 3.0 eq.) and TrtCl (4.04 g, 14.48 mmol, 1.0 eq.) in DMF (34 mL) were stirred at 20° C. for 12 hr. TLC (DCM/MeOH=10/1) showed the reaction was complete. The mixture was diluted with CH₂Cl₂, washed with H₂O (3*50 mL) and citric acid solution (3*50 mL). The organic phase was evaporated and purified by column chromatography (eluent: CH₂Cl₂/MeOH, 9:1). Compound aa29-2 (1.6 g, 4.21 mmol, 29.05% yield) was obtained as a white solid.

¹H NMR (400 MHz, CD₃OD) δ 7.57 (s, 1H), 7.51 (d, J=15.77 Hz, 1H), 7.38-7.45 (m, 9H), 7.32 (s, 1H), 7.15-7.22 (m, 6H), 6.45 (d, J=15.65 Hz, 1H) ppm.

### Step 2: Synthesis of 3-(1-trityl-1H-imidazol-5-yl) propanoic acid (aa29)

To a solution of compound aa29-2 (1.6 g, 4.21 mmol, 1 eq.) in EtOH (20 mL) was added Pd/C (1 g, 10% purity) under N₂ atmosphere. The suspension was degassed and purged with H₂ for 3 times. The mixture was stirred under H₂ (15 Psi.) at 20° C. for 2 h. After completion, the mixture was filtered and the filtrate was concentrated to give the product. Compound aa29 (1.2 g, 3.14 mmol, 74.60% yield) was obtained as a white solid.

LCMS (ESI): RT=0.802 min, mass calcd. for C₂₅H₂₁N₂O₂ 381.17[M−H]⁻, found 381.17 [M−H]⁻, Reverse phase LCMS was carried out using Waters Xbridge C18 30*2.0 mm, 3.5 μm, with a flow rate of 1.2 ml/min, eluting with a gradient of 5% to 95% acetonitrile containing ACN (solvent B) and water containing 0.05% NH₃H₂O in Water (solvent A).

¹H NMR (400 MHz, CD₃OD) δ 7.34-7.39 (m, 4H), 7.22-7.39 (m, 4H), 7.11-7.14 (m, 3H), 7.06-7.20 (m, 2H), 7.08 (d, J=7.25 Hz, 4H), 2.90 (t, J=7.32 Hz, 1H), 2.80 (t, J=7.38 Hz, 1H), 2.51-2.62 (m, 2H) ppm.

### 2.11 the Synthesis of Unnatural Amino Acid (Aa30)

Scheme 12 outlines the synthesis of unnatural amino acid (aa30):

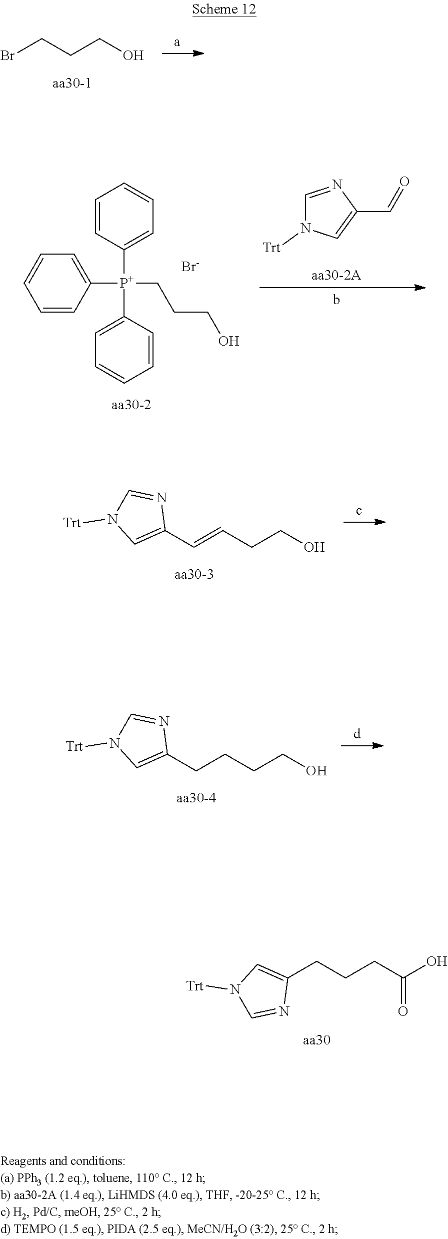

### Step 1: Synthesis of (3-hydroxypropyl)triphenylphosphonium bromide (aa30-2)

To a solution of 3-bromopropan-1-ol (10 g, 71.95 mmol, 6.49 mL, 1 eq.) in toluene (100 mL) was added PPh₃ (22.65 g, 86.34 mmol, 1.2 eq.). The mixture was stirred at 100° C. under N₂ for 12 h. The reaction progress was monitored by LCMS. After completion, the reaction was cooled to 0° C. and filtered, the filter cake was washed with toluene (10 mL*3), then dried under reduced pressure. Compound aa30-2 (20.7 g, crude) was obtained as a white solid.

LCMS (ESI): RT=0.758 min, mass calcd. for C₂₁H₂₂OP⁺, 321.14 [M]⁺, found 321.0 [M]⁺. LCMS conditions: 1.5 ML/4 L TFA in water (solvent A) and 0.75 ML/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm Wavelength: UV 220 nm & 254 nm; Column temperature: 50° C.; MS ionization: ESI.

¹H NMR (400 MHz, CD₃Cl) δ 7.83-7.65 (m, 15H), 4.94 (br s, 1H), 3.87-3.73 (m, 4H), 1.83 (m, 2H) ppm.

### Step 2: Synthesis of (E)-4-(1-trityl-1H-imidazol-4-yl)but-3-en-1-ol (aa30-3)

To a solution of aa30-2 (10.67 g, 26.60 mmol, 1.5 eq.) in THF (50 mL) was added LiHMDS (1 M, 70.92 mL, 4 eq.). The mixture was stirred at 0° C. for 0.5 h, then a solution of aa30-2A (1-tritylimidazole-4-carbaldehyde, 6 g, 17.73 mmol, 1.0 eq.) in THF (60 mL) was added to the above mixture and the resulting mixture was stirred at 25° C. for 12 h. The reaction progress was monitored by LCMS. After completion, the mixture was filtered and concentrated under reduced pressure to give a residue, then the residue was re-dissolved in dioxane (50 mL) and the final solution was stirred at 100° C. for 12 h, after that, the solution was concentrated under reduced pressure to give a residue. The residue was purified by flash silica gel chromatography (ISCO@; 80 g SepaFlash@ Silica Flash Column, Eluent of 0˜80% Ethylacetate/Petroleum ethergradient @ 60 mL/min). Compound aa30-3 (650 mg, crude) was obtained as a yellow solid.

LCMS (ESI): RT=0.846 min, mass calcd. for C₂₆H₂₄N₂ONa, 403.19 [M+Na]⁺, found 403.1 [M+Na]⁺. LCMS conditions: 1.5 ML/4 L TFA in water (solvent A) and 0.75 ML/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm Wavelength: UV 220 nm & 254 nm; Column temperature: 50° C.; MS ionization: ESI.

¹H NMR (400 MHz, CD₃OD) δ 7.68-7.57 (m, 1H), 7.40-7.37 (m, 9H), 7.18-7.13 (m, 6H), 6.83 (d, J=1.0 Hz, 1H), 6.36-6.14 (m, 2H), 3.62 (t, J=6.7 Hz, 2H), 2.36 (q, J=6.6 Hz, 2H) ppm.

### Step 3: Synthesis of 4-(1-trityl-1H-imidazol-4-yl)butan-1-ol (aa30-4)

To a solution of aa30-3 (650 mg, 1.71 mmol, 1 eq.) in MeOH (5 mL) was added Pd/C (50 mg, 489.61 mmol, 10% purity). The mixture was stirred at 25° C. for 2 h under H₂. The reaction progress was monitored by LCMS. After completion, the mixture was filtered and concentrated under reduced pressure to give a residue. The residue was purified by flash silica gel chromatography (ISCO@; 25 g SepaFlash@ Silica Flash Column, Eluent of 0˜50% Ethyl acetate/Petroleum ether gradient @ 40 mL/min) for 8 min with total volume. Compound aa30-4 (450 mg, 1.18 mmol, 68.87% yield, 100% purity) was obtained as a white solid.

LCMS (ESI): RT=0.850 min, mass calcd. for C₂₆H₂₆N₂ONa, 405.20 [M+Na]⁺, found 405.2 [M+Na]⁺. LCMS conditions: 1.5 ML/4 L TFA in water (solvent A) and 0.75 ML/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm Wavelength: UV 220 nm & 254 nm; Column temperature: 50° C.; MS ionization: ESI.

### Step 4: Synthesis of 4-(1-trityl-1H-imidazol-4-yl)butanoic acid (aa30)

To a solution of aa30-4 (450 mg, 1.18 mmol, 1 eq.) in MeCN (5 mL) and H₂O (7 mL) was added TEMPO (277.51 mg, 1.76 mmol, 1.5 eq.) and PIDA (lodobenzene diacetate (3240-34-4), 947.35 mg, 2.94 mmol, 2.5 eq.). The mixture was stirred at 25° C. for 12 h. The reaction progress was monitored by LCMS. After completion, to the mixture was added citric acid (aq. 15 mL) to adjust pH=4, then extracted with EtOAc (15 mL*2), the organic layers were collected, dried over Na₂SO₄, filtered, and concentrated under reduced pressure to give a residue. Then the residue was triturated by MTBE. Compound aa30 (250 mg, crude) was obtained as a white solid.

LCMS (ESI): RT=2.484 min, mass calcd. for C₂₆H₂₅N₂O₂, 397.18 [M+H]⁺, found 397.2 [M+H]⁺. LCMS conditions: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min. ESI source, Positive ion mode; Wavelength 220 nm & 254 nm, Oven Temperature 50° C.

¹H NMR (400 MHz, CD₃OD) δ 8.69 (d, J=1.6 Hz, 1H), 7.53-7.38 (m, 9H), 7.28-7.14 (m, 7H), 2.75 (t, J=7.5 Hz, 2H), 2.34 (t, J=7.1 Hz, 2H), 2.00-1.87 (m, 2H) ppm.

### 2.12 the Synthesis of Unnatural Amino Acid (Aa31)

Scheme 13 outlines the synthesis of unnatural amino acid (aa31):

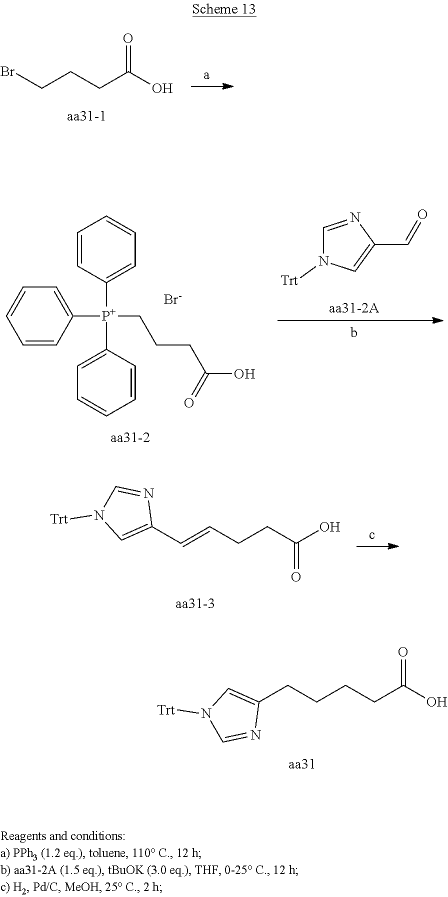

### Step 1: Synthesis of (3-carboxypropyl)triphenylphosphonium bromide (aa31-2)

To a solution of 4-bromobutanoic acid (5 g, 29.94 mmol, 6.76 mL, 1 eq.) in toluene (70 mL) was added PPh3 (8.64 g, 32.93 mmol, 1.1 eq.). The mixture was stirred at 110° C. under N2 for 12 h. The reaction progress was monitored by TLC and LCMS. Upon completion, the reaction mixture was cooled to 0° C., then filtered and washed with toluene (10 mL*3), the filter cake was collected and dried under reduced pressure. Compound aa31-2 (5.6 g, crude) was obtained as a white solid.

LCMS (ESI): RT=1.076 min, mass calcd. for C₂₂H₂₂O₂P⁺, 349.14[M]⁺, found 349.2 [M]⁺. LCMS conditions: 1.5 ML/4 L TFA in water (solvent A) and 0.75 ML/4 L TFA in acetonitrile (solvent B), using the elution gradient 10%-80% (solvent B) over 1.35 minutes and holding at 80% for 0.9 minutes at a flow rate of 0.8 ml/min; Column: Xtimate C18 2.1*30 mm, 3 mm; Wavelength: UV 220 nm & 254 nm Column temperature: 50° C.; MS ionization: ESI.

¹H NMR (400 MHz, CD₃Cl) δ 7.91-7.66 (m, 15H), 3.31-3.22 (m, 2H), 2.55 (t, J=6.3 Hz, 2H), 1.84 (m, 2H) ppm.

### Step 2: Synthesis of (E)-5-(1-trityl-1H-imidazol-4-yl)pent-4-enoic acid (aa31-3)

To a solution of aa31-2 (3.81 g, 8.87 mmol, 1.5 eq.) in THF (20 mL) was added tBuOK (1.99 g, 17.73 mmol, 3 eq.) at 0° C. under N₂, the mixture was stirred at 0° C. for 30 min, then a solution of aa31-2A (2 g, 5.91 mmol, 1 eq.) in THF (20 mL) was added to the above mixture at 0° C. and the final mixture was stirred at 25° C. for 12 h. The reaction progress was monitored by LCMS. Upon completion, to the mixture was added citric acid to adjust pH=4, then extracted with EtOAc (50 mL*2), the organic layers were collected, dried over Na₂SO₄, filtered, and concentrated under reduced pressure to give a residue. The residue was purified by flash silica gel chromatography (ISCO@; 80 g SepaFlash@ Silica Flash Column, Eluent of 0˜10% MeOH/DCM@ 40 mL/min). Compound aa31-3 (4 g, crude) was obtained as a light yellow foam.

LCMS (ESI): RT=1.487 min, mass calcd. for C₂₇H₂₅N₂O₂, 409.18 [M+H]⁺, found 409.3 [M+H]⁺. LCMS conditions: 1.5 ML/4 L TFA in water (solvent A) and acetonitrile (solvent B), using the elution gradient 10%-80% (solvent B) over 1.35 minutes and holding at 80% for 0.9 minutes at a flow rate of 0.8 ml/min; Column: Xtimate C18 2.1*30 mm, 3 mm; Wavelength: UV 220 nm & 254 nm; Column temperature: 50° C.; MS ionization: ESI.

### Step 3: Synthesis of 5-(1-trityl-1H-imidazol-4-yl)pentanoic acid (aa31)

To a solution of aa31-3 (3 g, 7.34 mmol, crude purity, 1 eq.) in MeOH (40 mL) was added Pd/C (300 mg, 489.61 mmol, 10% purity). The mixture was stirred at 25° C. for 2 h under H2. The reaction progress was monitored by LCMS. Upon completion, the mixture was filtered and concentrated under reduced pressure to give a residue. The residue was purified by flash silica gel chromatography (ISCO@; 40 g SepaFlash@ Silica Flash Column, Eluent of 0˜10% MeOH/DCM@40 mL/min) for 7 min with total volume. After that, the product was triturated by TBME. Compound aa31 (290 mg, 692.32 mmol, 71.05% yield, 98% purity) was obtained as a white solid.

LCMS (ESI): RT=1.805 min, mass calcd. for C₂₇H₂₇N₂O₂, 411.20 [M+H]⁺, found 411.3 [M+H]⁺. LCMS conditions: 0.8 mL/4 L NH₃·H₂O in water (solvent A) and acetonitrile (solvent B), using the elution gradient 10%-80% (solvent B) over 6 minutes and holding at 80% for 0.5 minutes at a flow rate of 0.8 ml/min; Column: XBridge C18 3.5 mm 2.1*30 mm; Wavelength: UV 220 nm & 254 nm; Column temperature: 50° C.; MS ionization: ESI.

¹H NMR (400 MHz, CD₃Cl) δ 7.65 (s, 1H), 7.44-7.34 (m, 9H), 7.16 (dd, J=2.9, 6.8 Hz, 6H), 6.77 (s, 1H), 2.58 (br t, J=6.8 Hz, 2H), 2.28 (t, J=7.1 Hz, 2H), 1.70-1.55 (m, 4H) ppm.

### 2.13 the Synthesis of Unnatural Amino Acid (Aa32)

Scheme 14 outlines the synthesis of unnatural amino acid (aa32):

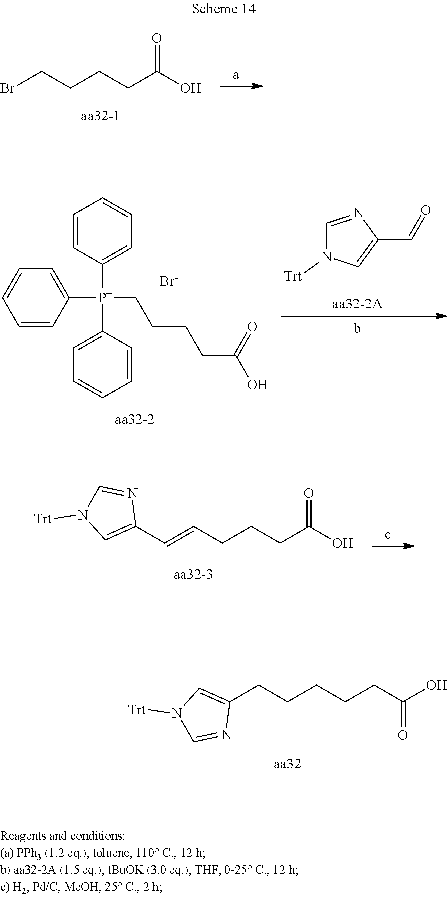

### Step 1: Synthesis of (4-carboxybutyl)triphenylphosphonium bromide (aa32-2)

To a solution of 5-bromopentanoic acid (10 g, 55.24 mmol, 6.76 mL, 1 eq.) in toluene (100 mL) was added PPh₃ (15.94 g, 60.76 mmol, 1.1 eq.). The mixture was stirred at 110° C. for 12 h under N₂. The reaction progress was monitored by LCMS. Upon completion, the reaction mixture was cooled to 0° C., then filtered and washed with toluene (10 mL*3), the filter cake was collected and dried under reduced pressure. Compound aa32-2 (16.4 g, 36.99 mmol, 66.97% yield) was obtained as a white solid.

LCMS (ESI): RT=1.076 min, mass calcd. for C₂₂H₂₂O₂P⁺, 349.14 [M+H]⁺, found 349.2 [M+H]⁺. LCMS conditions: 1.5 ML/4 L TFA in water (solvent A) and 0.75 ML/4 L TFA in acetonitrile (solvent B), using the elution gradient 10%-80% (solvent B) over 1.35 minutes and holding at 80% for 0.9 minutes at a flow rate of 0.8 ml/min; Column: Xtimate C18 2.1*30 mm, 3 mm; Wavelength: UV 220 nm & 254 nm Column temperature: 50° C.;

¹H NMR (400 MHz, CD₃Cl) δ 7.91-7.66 (m, 15H), 3.31-3.22 (m, 2H), 2.55 (t, J=6.3 Hz, 2H), 1.86-1.82 (m, 4H) ppm.

### Step 2: Synthesis of (E)-6-(1-trityl-1H-imidazol-4-yl)hex-5-enoic acid (aa32-3)

To a solution of aa32-2 (7.86 g, 17.73 mmol, 1.5 eq.) in THF (60 mL) was added t-BuOK (3.98 g, 35.46 mmol, 3 eq.) at 0° C. under N₂. The mixture was stirred at 0° C. for 30 min. A solution of aa32-2A (4 g, 11.82 mmol, 1 eq.) in THF (40 mL) was added to the above mixture at 0° C. and the resulting mixture was stirred at 25° C. for 12 h. The reaction progress was monitored by LCMS. Upon completion, to the mixture was added citric acid to adjust pH=4, then extracted with EtOAc (50 mL*2), the organic layers were collected, dried over Na₂SO₄, filtered, and concentrated under reduced pressure to give a residue. The residue was purified by flash silica gel chromatography (ISCO@; 80 g SepaFlash@ Silica Flash Column, Eluent of 0˜10% MeOH/DCM@40 mL/min). Compound aa32-3 (4 g, crude) was obtained as a light yellow foam.

LCMS (ESI): RT=2.739 min, mass calcd. for C₂₈H₂₇N₂O₂, 423.20[M+H]⁺, found 423.2 [M+H]⁺. LCMS conditions: 1.5 ML/4 L TFA in water (solvent A) and acetonitrile (solvent B), using the elution gradient 10%-80% (solvent B) over 1.35 minutes and holding at 80% for 0.9 minutes at a flow rate of 0.8 ml/min; Column: Xtimate C18 2.1*30 mm, 3 mm; Wavelength: UV 220 nm & 254 nm; Column temperature: 50° C.; MS ionization: ESI.

### Step 3: Synthesis of 6-(1-trityl-1H-imidazol-4-yl)hexanoic acid (aa32)

To a solution of aa32-3 (4 g, 9.47 mmol, 1 eq.) in MeOH (40 mL) was added Pd/C (300 mg, 489.61 mmol, 10% purity). The mixture was stirred at 25° C. for 2 h under H₂. The reaction progress was monitored by LCMS. Upon completion, the mixture was filtered and concentrated under reduced pressure to give a residue. The residue was purified by flash silica gel chromatography (ISCO@; 40 g SepaFlash@ Silica Flash Column, Eluent of 0˜10% MeOH/DCM@40 mL/min). After that, the product was triturated by TBME. Compound aa32 (660 mg, 1.51 mmol, 15.93% yield, 97% purity) was obtained as a white solid.

LCMS (ESI): RT=2.807 min, mass calcd. for C₂₈H₂₉N₂O₂, 425.22 [M+H]⁺, found 425.2 [M+H]⁺. LCMS conditions: 1.5 ML/4 L TFA in water (solvent A) and 0.75 ML/4 L TFA in acetonitrile (solvent B), using the elution gradient 10%-80% (solvent B) over 6.0 minutes and holding at 80% for 0.5 minutes at a flow rate of 0.8 ml/min; Column: Xtimate C18 2.1*30 mm, 3 mm; Wavelength: UV 220 nm & 254 nm Column temperature: 50° C.; MS ionization: ESI.

¹H NMR (400 MHz, CD₃OD) δ 7.43 (d, J=1.3 Hz, 1H), 7.41-7.34 (m, 9H), 7.19-7.11 (m, 6H), 6.65 (s, 1H), 2.52 (t, J=7.4 Hz, 2H), 2.24 (t, J=7.4 Hz, 2H), 1.60 (m, 4H), 1.38-1.27 (m, 2H) ppm.

### 2.14 the Synthesis of Unnatural Amino Acid (Aa33)

Scheme 15 outlines the synthesis of unnatural amino acid (aa33):

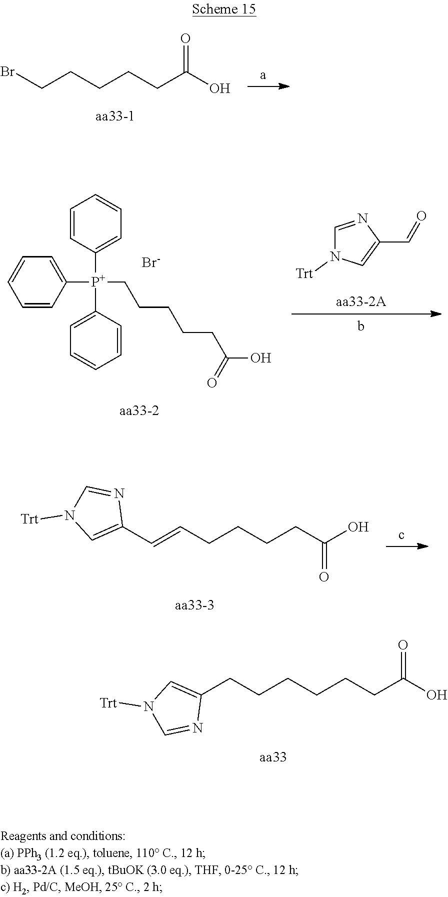

### Step 1: Synthesis of (5-carboxypentyl)triphenylphosphonium bromide (aa33-2)

To a solution of 6-bromohexanoic acid (5 g, 25.63 mmol, 1 eq.) in toluene (50 mL) was added PPh₃ (7.06 g, 26.92 mmol, 1.05 eq.). The resulting solution was stirred at 120° C. over 12 h. After completion, the reaction mixture was cooled to 0° C., then filtered and washed with toluene (10 mL*3), the filter cake was collected and dried under reduced pressure. Compound aa33-2 (6.85 g, 14.47 mmol, 56.44% yield, 96.6% purity) was obtained as a white solid.

LCMS (ESI): RT=0.916 min, m/z calcd. for C₂₄H₂₆PO₂⁺ 377.17, found 377.1 [M-Br]*. Reverse phase LCMS was carried out using a Xtimate C18, 2.1*30 mm 3 mm, SN: 3U411301530 column, with a flow rate of 1.5 mL/min, eluting with a gradient of 10% to 80% acetonitrile containing 0.02% TFA (solvent B) and water containing 0.04% TFA (solvent A).

### Step 2: Synthesis of (E)-7-(1-trityl-1H-imidazol-4-yl)hept-6-enoic acid (aa33-3)

To a solution of aa33-2 (4.05 g, 8.87 mmol, 2 eq.) in THF (20 mL) was added tBuOK (1 M, 22.16 mL, 5 eq.) at 0° C. under N₂, the mixture was stirred at 0° C. for 30 min, then a solution of aa33-2A (1.5 g, 4.43 mmol, 1 eq.) in THF (20 mL) was added to the above mixture at 0° C. and the final mixture was stirred at 25° C. for 12 h. The reaction progress was monitored by LCMS. After completion, the mixture was added citric acid to adjust pH=4, then extracted with EtOAc (50 mL*2), the organic layers were collected, dried over Na₂SO₄, filtered and concentrated under reduced pressure to give a residue. The crude product was used into the next step without any further purification. Compound aa33-3 (3.8 g, crude) was obtained as a yellow syrup.

LCMS (ESI): RT=2.904 min and 3.068 min, mass calcd. for C₂₉H₂₉N₂O₂ 437.22, found 437.3 [M+H]⁺; Reverse phase LCMS was carried out using a Chromolith Flash RP-C18 25-3 mm column, with a flow rate of 1.5 mL/min, eluting with a gradient of 10% to 80% acetonitrile containing 0.02% TFA (solvent B) and water containing 0.04% TFA (solvent A).

### Step 3: Synthesis of 7-(1-trityl-1H-imidazol-4-yl)heptanoic acid (aa33)

To a solution of aa33-3 (3 g, 6.87 mmol, 1 eq.) in MeOH (30 mL) was added Pd/C (300 mg, 489.61 mmol, 10% purity). The mixture was stirred at 25° C. for 2 h under H₂. The reaction progress was monitored by LCMS. After completion, the residue was purified by prep-HPLC (column: Boston Prime C18 150*25 mm*5 mm; mobile phase: [water (0.05% ammonia hydroxide v/v)-ACN]; B %: 22%-45%, 7 min). Compound aa33 (200 mg, 456.04 mmol, 6.64% yield, 100% purity) was obtained as a white solid.

LCMS (ESI): RT=2.231 min, mass calcd. for C₂₉H₃₁N₂O₂, 439.23 [M+H]⁺, found 439.3 [M+H]⁺. LCMS conditions: Mobile Phase: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min. ESI source, Positive ion mode; Wavelength 220 nm & 254 nm, Oven Temperature 50° C.

¹H NMR (400 MHz, CD₃OD) δ 7.45-7.31 (m, 10H), 7.19-7.11 (m, 6H), 6.64 (s, 1H), 2.52 (t, J=7.3 Hz, 2H), 2.24 (t, J=7.4 Hz, 2H), 1.65-1.52 (m, 4H), 1.38-1.26 (m, 4H) ppm.

### 2.15 the Synthesis of Unnatural Amino Acid (Aa34)

Scheme 16 outlines the synthesis of unnatural amino acid (aa34):

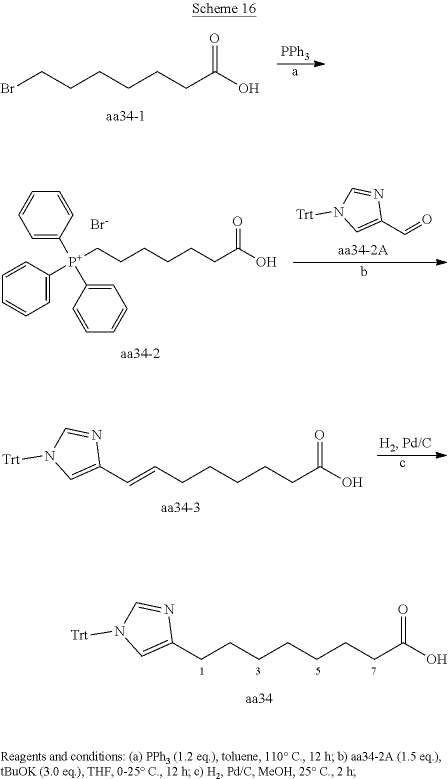

### Step 1: Synthesis of 7-[BLAH(triphenyl)-A5-phosphanyl]heptanoic acid (aa34-2)

To a solution of aa34-1 (10 g, 47.83 mmol, 1 eq) in toluene (100 mL) was added PPhs (13.80 g, 52.61 mmol, 1.1 eq). The mixture was stirred at 110° C. for 12 hr. After completion, the reaction was filtered under reduced pressure to give a residue. The crude product was used to the next step without further purification. Compound aa34-2 (22.8 g, 43.73 mmol, 91.43% yield, 90.410% purity) was obtained as a yellow oil.

LCMS (ESI): RT=0.792 min, m/z calcd. for C₂₅H₂₈O₂P⁺ 391.18 [M]⁺, found 391.1 [M]⁺, LC-MS Conditions: Mobile Phase: 1.5 ML/4 L TFA in water (solvent A) and 0.75 ML/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm.

### Step 2: Synthesis of (E)-8-(1-tritylimidazol-4-yl)oct-7-enoic acid (aa34-3)

To a solution of aa34-2 (10.45 g, 22.16 mmol, 1.5 eq.) in THF (150 mL) was added t-BuOK (4.97 g, 44.33 mmol, 3 eq) at 0° C. under N₂, the mixture was stirred at 0° C. then a solution of aa34-2A (5 g, 14.78 mmol, 1 eq) in THF (20 mL) added to the above mixture at 0° C. and the final mixture was stirred at 25° C. for 12 hr. After completion, the mixture was added citric acid to adjust pH=4, then extracted with EtOAc (200 mL*2), the organic layers were collected, dried over Na₂SO₄, filtered and concentrated under reduced pressure to give the target aa34-3 (14.5 g, crude) as a white solid.

LCMS (ESI): RT=0.870 min, m/z calcd. for C₃₀H₃₁N₂O₂ 451.23 [M+H]⁺, C₃₀H₃₀N₂⁺O₂Na 473.23 [M+Na]⁺, found 473.1 [M+Na]⁺, LC-MS Conditions: Mobile Phase: 1.5 ML/4 L TFA in water (solvent A) and 0.75 ML/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm.

### Step 3: Synthesis of 8-(1-tritylimidazol-4-yl)octanoic acid (aa34)

To a solution of aa34-3 (14 g, 15.54 mmol, 50% purity, 1 eq) in MeOH (200 mL) was added Pd/C (4 g, 10% purity) and H₂. The mixture was stirred at 25° C. for 3 hr. After completion, the mixture was filtered and concentrated under reduced pressure to give a residue. The crude product was purified by C-18 reverse phase chromatography (ISCO@; 120 g SepaFlash@ C-18 Column, Eluent of 60˜70% acetonitrile/H₂O gradient @ 80 mL/min, 40 min with total volume 2 L) to give a residue (neutral). Compound aa34 (2.1 g, 4.53 mmol, 29.15% yield, 97.6% purity) was obtained as a white solid.

LCMS (ESI): RT=2.956 min, mass calcd. for C₃₀H₃₃N₂O₂ 453.25 [M+H]⁺, found 453.3 [M+H]⁺. LCMS conditions: 1.5 ML/4 L TFA in water (solvent A) and 0.75 ML/4 L TFA in acetonitrile (solvent B), using the elution gradient 10%-80% (solvent B) over 6.0 minutes and holding at 80% for 0.5 minutes at a flow rate of 0.8 ml/min; Column: Xtimate C18 2.1*30 mm, 3 μm; Wavelength: UV 220 nm & 254 nm Column temperature: 50° C.; MS ionization: ESI.

¹H NMR (400 MHz, CD₃OD) δ 7.47-7.33 (m, 10H), 7.22-7.10 (m, 6H), 6.64 (s, 1H), 2.52 (t, J=7.4 Hz, 2H), 2.26 (t, J=7.4 Hz, 2H), 1.69-1.48 (m, 4H), 1.37-1.26 (m, 6H) ppm.

### 2.16 the Synthesis of Unnatural Amino Acid (Aa35)

Scheme 17 outlines the synthesis of unnatural amino acid (aa35):

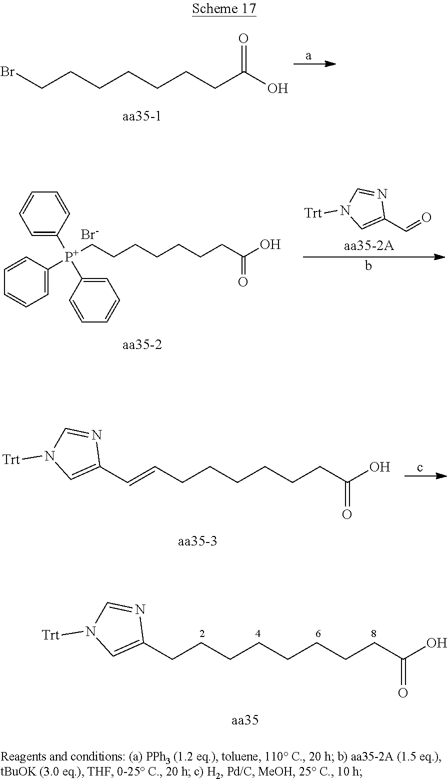

### Step 1: Synthesis of (7-carboxyheptyl)triphenylphosphonium bromide (aa35-2)

To a solution of aa35-1 (15 g, 71.74 mmol, 1 eq.) in toluene (150 mL) was added PPh₃ (20.70 g, 78.92 mmol, 1.1 eq.). The mixture was stirred at 110° C. under N₂ for 20 h. After completion, the reaction mixture was cooled to 0° C., then filtered and washed with toluene (10 mL*3), the filter cake was collected and dried under reduced pressure. The residue was triturated with THF (200 mL). Then the mixture was filtered, and the filter cake was dried to give the product aa35-2 (20 g, 40.31 mmol, 56.18% yield, 95% purity) as a white solid.

LCMS: (ESI): Rt=2.175 min, mass calcd. for C₂₆H₃₀O₂P⁺[M+H]⁺405.20, found 405.70; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

### Step 2: Synthesis of (E)-9-(1-trityl-1H-imidazol-4-yl)non-8-enoic acid (aa35-3)

To a solution of aa35-2 (10.45 g, 22.17 mmol, 1.5 eq.) in THF (150 mL) was added t-BuOK (4.97 g, 44.34 mmol, 3.0 eq.) at 0° C. Then the mixture was stirred at 0° C. for 30 min. A solution of aa35-2A (5 g, 14.78 mmol, 1 eq.) in THF (50 mL) was added. Then the mixture was stirred at 20° C. for 12 h. After completion, the reaction mixture was quenched by addition H2O (100 mL) at 0° C., and then extracted with EtOAc (150 mL*2). The combined organic layers were washed with Sat. NaCl 100 mL (50 mL*2), dried over anhydrous sodium sulfate, filtered, and concentrated under reduced pressure to give a residue. The residue was purified by flash silica gel chromatography (ISCO@; 80 g Special Flash® Silica Flash Column, Eluent of 0˜20% Ethyl acetate/Petroleum ether gradient @ 60 mL/min) to give the product aa35-3 (1.4 g, crude) as a light-yellow oil.

LCMS: (ESI): Rt=3.310 min, mass calcd. for C₃₁H₃₃N₂O₂ [M+H]⁺ 465.25, found 465.20 [M+H]⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

### Step 3: Synthesis of 9-(1-tritylimidazol-4-yl)nonanoic acid (aa35)

To a solution of aa35-3 (1.4 g, 3.01 mmol, 1 eq.) in MeOH (40 mL) was added Pd/C (0.2 g, 10% purity). The mixture was stirred at 25° C. under H₂ for 3 hr. After completion, the mixture was filtered and concentrated under reduced pressure to give a residue. The crude product was purified by prep-HPLC (column: C18 spherical 20-35 um, 100A, 12 g; mobile phase: [Water-ACN]; B %: 0%-90%, 15 min) to give the product aa35 (0.12 g, 244.31 μmol, 8.11% yield, 95% purity) was obtained as a white solid.

LCMS: (ESI): Rt=3.257 min, mass calcd. for C₃₁H₃₅N₂O₂ [M+H]⁺467.26, found 467.20 [M+H]⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

### 2.17 the Synthesis of Unnatural Amino Acid (Aa36)

Scheme 18 outlines the synthesis of unnatural amino acid (aa36):

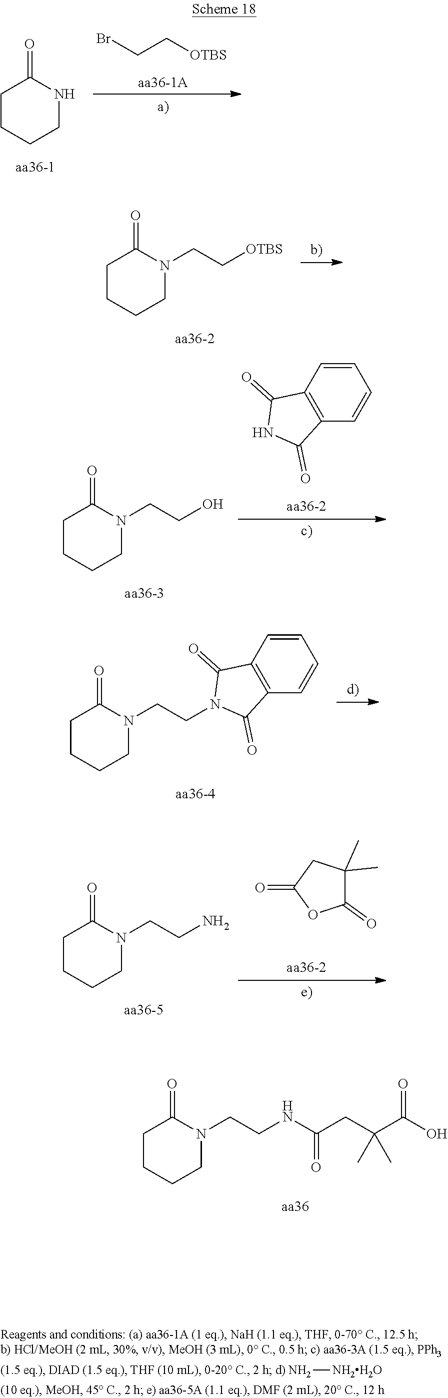

### Step 1: Synthesis of 1-(2-((tert-butyldimethylsilyl)oxy)ethyl)piperidin-2-one (aa36-2)

To a solution of aa36-1 (6 g, 60.53 mmol, 1 eq) in THF (300 mL) was added NaH (2.66 g, 66.58 mmol, 60% purity, 1.1 eq) in portions at 0° C. The mixture was stirred at 20° C. for 0.5 h and aa36-1A (14.48 g, 60.53 mmol, 1 eq) was added. The reaction mixture was heated to 70° C. for 12 h. LCMS showed the desired product was observed. The mixture was cooled to r.t., quenched by water (150 mL) and extracted with EtOAc (150 mL×2). The combined organic layers were dried over anhydrous Na₂SO₄, filtered and concentrated in vacuo. The residue was purified by column chromatography (SiO₂, Petroleum ether/Ethyl acetate=1/1). Compound aa36-2 (2.7 g, 10.49 mmol, 17.33% yield) was obtained as a colorless oil.

LCMS (ESI): RT=0.974 min, mass calcd. for C₁₃H₂₇NO₂SiH 258.18, found 258.2 [M+H]⁺; Reverse phase LCMS was carried out using a Chromolith Flash Agilent Pursult 5 C18 20*2.0 mm, with a flow rate of 1.5 mL/min, eluting with a gradient of 5%-95% (solvent B) over 0.7 minutes and holding at 95% for 0.4 minutes.

¹H NMR (400 MHz, DMSO-d6) 3.64 (t, J=6.0 Hz, 2H), 3.32-3.29 (m, 4H), 2.16 (t, J=6.0 Hz, 2H), 1.74-1.57 (m, 4H), 0.83 (s, 9H), 0.00 (s, 6H) ppm.

### Step 2: Synthesis of 1-(2-hydroxyethyl)piperidin-2-one (aa36-3)

To a solution of aa36-2 (2.7 g, 10.49 mmol, 1 eq) in MeOH (12 mL) was added a solution of HCl/MeOH (v/v=30%, 8 mL) at 0° C. The solution was stirred at 0° C. for 0.5 h. After completion, the solution was concentrated in vacuo. The residue was purified by column chromatography (SiO₂, DCM/MeOH=10/1). The crude product aa36-3 (1.4 g, 9.73 mmol, 92.76% yield, 99.5% purity) was obtained as a yellow solid which was used into the next step without further purification.

¹H NMR (400 MHz, DMSO-d6) 4.13-4.11 (m, 1H), 3.52-3.44 (m, 2H), 3.34-3.29 (m, 4H), 2.20 (t, J=6.0 Hz, 2H), 1.75-1.61 (m, 4H) ppm.

LCMS (ESI): RT=0.448-0.574 min, mass calcd. for C₇H₁₃NO₂H 144.09, found 144.2 [M+H]⁺; Reverse phase LCMS was carried out using a Chromolith Flash Agilent Pursult 5 C18 20*2.0 mm, with a flow rate of 1.5 mL/min, eluting with a gradient of 5%-95% (solvent B) over 0.7 minutes and holding at 95% for 0.4 minutes.

### Step 3: Synthesis of 2-(2-(2-oxopiperidin-1-yl)ethyl)isoindoline-1,3-dione (aa36-4)

To a solution of aa36-3 (1.5 g, 10.48 mmol, 1 eq) and aa36-3A (1.85 g, 12.57 mmol, 1.2 eq) in THF (40 mL) was added PPh₃(4.12 g, 15.71 mmol, 1.5 eq) and DIAD (3.18 g, 15.71 mmol, 3.06 mL, 1.5 eq) at 0° C. The resulting mixture was stirred at 20° C. for 2 h. LCMS showed the desired product was observed. The mixture was filtered, and the filtrate was concentrated in vacuo. The residue was purified by column chromatography (SiO₂, Petroleum ether/Ethyl acetate=1/1). Compound aa36-4 (0.15 g, 550.87 μmol, 5.26% yield) was obtained as a white solid.

LCMS (ESI): RT=0.764 min, mass calcd. for C₁₅H₁₆N₂O₃H 273.12, found 273.1 [M+H]⁺; Reverse phase LCMS was carried out using a Chromolith Flash Agilent Pursult 5 C18 20*2.0 mm, with a flow rate of 1.5 mL/min, eluting with a gradient of 5%-95% (solvent B) over 0.7 minutes and holding at 95% for 0.4 minutes.

¹H NMR (400 MHz, DMSO-d6) 7.83-7.65 (m, 4H), 3.68-3.58 (m, 2H), 3.45-3.39 (m, 2H), 3.21 (t, J=6.0 Hz, 2H), 1.89 (t, J=6.4 Hz, 2H), 1.64-1.56 (m, 2H), 1.55-1.47 (m, 2H) ppm.

### Step 4: Synthesis of 1-(2-aminoethyl)piperidin-2-one (aa36-5)

To a solution of aa36-4 (0.15 g, 550.87 μmol, 1 eq) in MeOH (10 mL) was added NH₂NH₂—H₂O (275.76 mg, 5.51 mmol, 267.73 μL, 10 eq). The solution was stirred at 45° C. for 2 h. After completion, the solution was cooled to r.t. and concentrated in vacuo. The crude product aa36-5 (0.07 g, crude) was obtained as a colorless oil, which was used into the next step without further purification.

¹H NMR (400 MHz, DMSO-d6) 3.27-3.22 (m, 6H), 2.62 (t, J=6.8 Hz, 2H), 2.17 (t, J=6.0 Hz, 2H), 1.76-1.58 (m, 4H) ppm.

### Step 5: Synthesis of 2,2-dimethyl-4-oxo-4-((2-(2-oxopiperidin-1-yl)ethyl)amino)butanoic acid (aa36)

To a solution of aa36-5 (0.07 g, 492.27 μmol, 1 eq) in DMF (2 mL) was added aa36-5A (69.38 mg, 541.50 μmol, 60.86 μL, 1.1 eq). The solution was stirred at 20° C. for 12 h. LCMS showed the desired product was observed. The solution was concentrated in vacuo. The residue was purified by prep-HPLC (FA condition; column: Phenomenex Synergi C18 150*30 mm*4 um; mobile phase: [water (0.225% FA)-ACN]; B %: 15%-25%, 11 min). Compound aa36 (20 mg, 73.99 μmol, 15.03% yield) was obtained as a white solid.

LCMS (ESI): RT=0.19 min, mass calcd. for C₁₃H₂₂N₂O₄H 271.16, found 271.1 [M+H]⁺; Reverse phase LCMS was carried out using a Chromolith Flash Agilent Pursult 5 C18 20*2.0 mm, with a flow rate of 1.5 mL/min, eluting with a gradient of 5%-95% (solvent B) over 0.7 minutes and holding at 95% for 0.4 minutes.

¹H NMR (400 MHz, METHANOL-d4) 3.51-3.44 (m, 2H), 3.43-3.35 (m, 4H), 2.47 (s, 2H), 2.35 (t, J=6.0 Hz, 2H), 1.91-1.76 (m, 4H), 1.25 (s, 6H) ppm.

### 2.18 the Synthesis of Unnatural Amino Acid (Aa37)

Scheme 19 outlines the synthesis of unnatural amino acid (aa37):

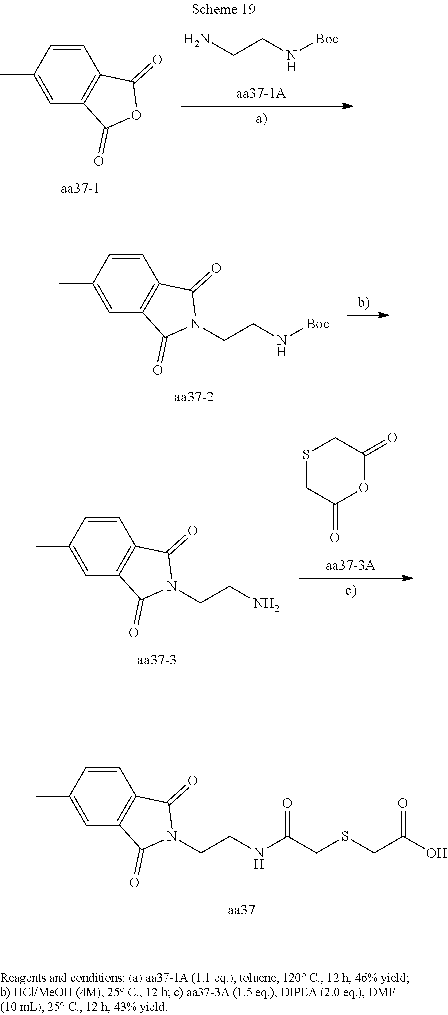 [SMILES: Cc1ccc2c(c1)C(=O)OC2=O ; CC(C)(C)OC(=O)NCCN ; Cc1ccc2c(c1)C(=O)N(CCNC(=O)OC(C)(C)C)C2=O ; Cc1ccc2c(c1)C(=O)N(CCN)C2=O ; O=C1CSCC(=O)O1 ; Cc1ccc2c(c1)C(=O)N(CCNC(=O)CSCC(=O)O)C2=O]

### Step 1: Synthesis of tert-butyl N-[2-(5-methyl-1,3-dioxo-isoindolin-2-yl)ethyl]carbamate (aa37-2)

To a solution of aa37-1 (500 mg, 3.08 mmol, 1 eq) in toluene (10 mL) was added aa37-1A (543.46 mg, 3.39 mmol, 532.80 μL, 1.1 eq). The mixture was stirred at 120° C. for 12 hr. After completion, the reaction mixture was concentrated under vacuum to give the crude product, which was triturated with MTBE/PE (1:1) at 25° C. for 1 h. Compound aa37-2 (460 mg, 1.44 mmol, 46.56% yield, 95% purity) was obtained as a yellow solid.

LCMS (ESI): RT=0.898 min, m/z calcd. for C₁₆H₂₁N₂O₄ 305.14 [M+H]⁺, C₁₆H₂₁N₂O₄Na 327.14 [M+Na]⁺, found 327.2 [M+Na]⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

### Step 2: Synthesis of 2-(2-aminoethyl)-5-methyl-isoindoline-1,3-dione (aa37-3)

To a solution of aa37-2 (460 mg, 1.51 mmol, 1 eq) was added HCl/MeOH (4 M, 377.87 μL, 1 eq). The mixture was stirred at 25° C. for 12 hr. After completion, the reaction was concentrated in vacuum to give the crude product aa37-3 (300 mg, crude, HCl salt) was obtained as a white solid.

LCMS (ESI): RT=0.638 min, m/z calcd. for C₁₁H₁₃N₂O₂ 205.09 [M+H]⁺, found 205.0 [M+H]⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

¹H NMR (400 MHz, CHLOROFORM-d) δ=7.99 (br s, 2H), 7.26-7.21 (m, 1H), 3.56 (br s, 2H), 2.77 (br s, 4H), 2.08 (s, 3H) ppm.

### Step 3: Synthesis of 2-[2-[2-(5-methyl-1,3-dioxo-isoindolin-2-yl)ethylamino]-2-oxo-ethyl]sulfanylacetic acid (aa37)

To a solution of aa37-3 (250 mg, 1.22 mmol, 1 eq) and aa37-3A (194.11 mg, 1.47 mmol, 1.2 eq) in DMF (4 mL) was added DIPEA (316.42 mg, 2.45 mmol, 426.45 μL, 2 eq). The mixture was stirred at 25° C. for 12 hr. The reaction mixture was partitioned between EtOAc (50 mL) and water (60 mL). The water layer was acidified to pH=4 by 1 N HCl solution. The organic phase was separated, washed with brine (30 mL×3), and dried over anhydrous Na₂SO₄. The resulting solution was concentrated under reduced pressure. The residue was purified by flash silica gel chromatography (ISCO@; 12 g SepaFlash@ Silica Flash Column, Eluent of 0˜30% Ethyl acetate/Petroleum ether gradient @ 35 mL/min). Compound aa37 (200 mg, 535.14 μmol, 43.72% yield, 90% purity) was obtained as a white solid.

LCMS (ESI): RT=0.757 min, m/z calcd. for C₁₅H₁₆N₂O₅SNa 359.08 [M+Na]⁺, found 358.9 [M+Na]⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

¹H NMR (400 MHz, CHLOROFORM-d) δ=7.69-7.60 (m, 1H), 7.55 (s, 1H), 7.43 (br d, J=7.3 Hz, 1H), 3.82-3.70 (m, 2H), 3.52-3.41 (m, 2H), 2.86 (s, 2H), 2.76 (s, 2H), 2.38 (s, 3H) ppm.

### Example 3. Synthesis of GLP1 Peptidomimetics

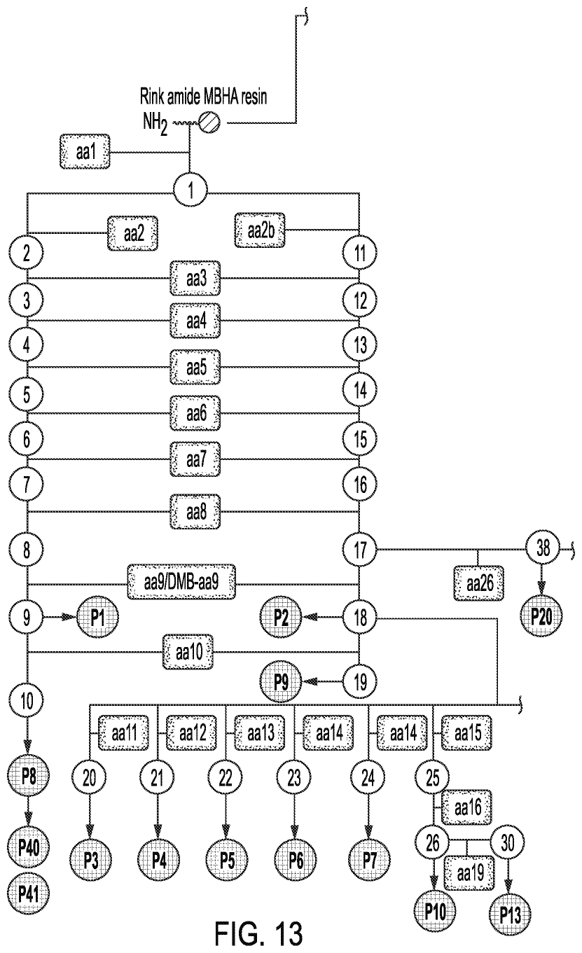

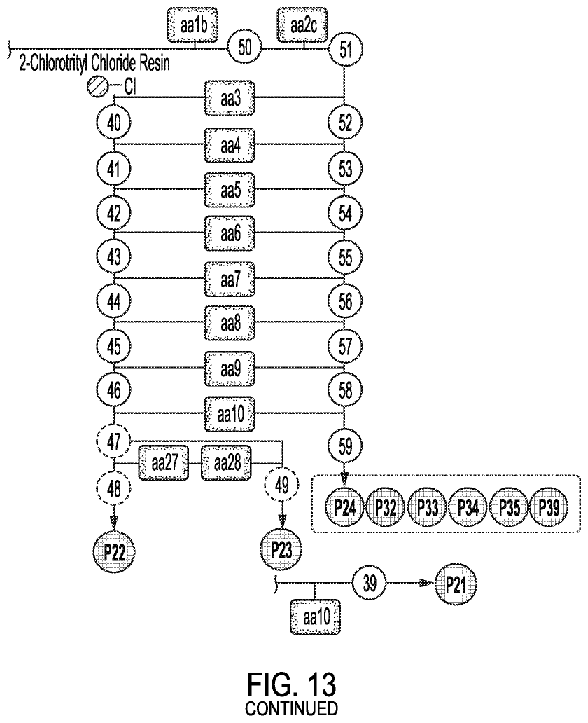

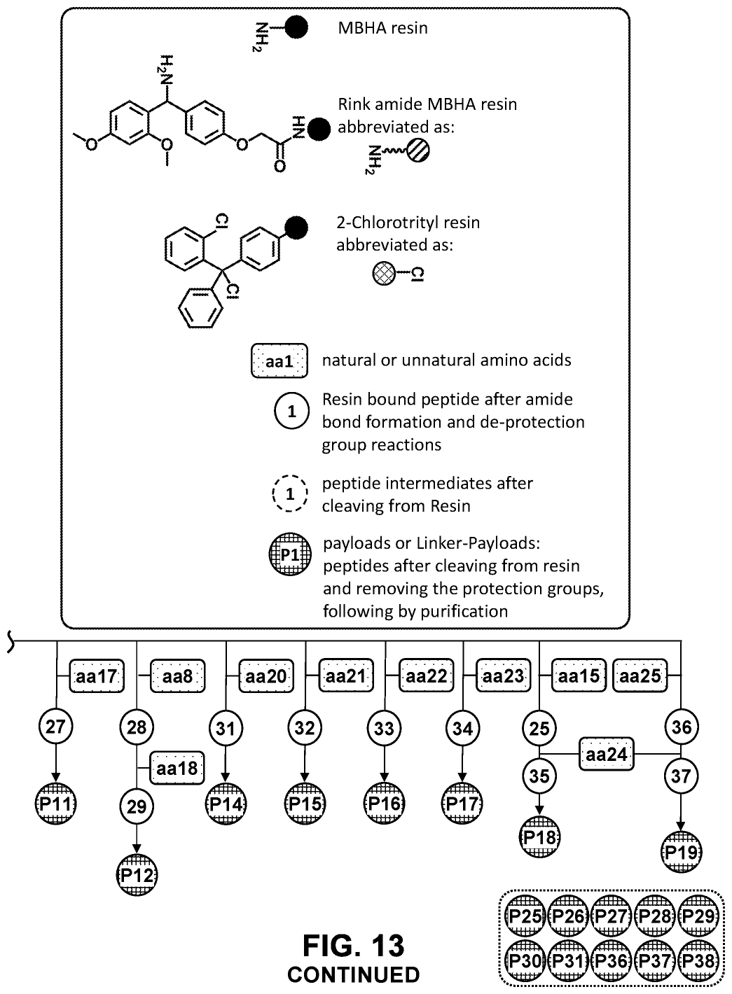

The general synthetic scheme for making GLP1 peptidomimetic payloads according to the present disclosure is shown as FIG. 13. Table 2, shown below, depicts the structures of Rink amide MBHA resin bound peptides and intermediates 1-59.

TABLE 2
Rink amide MBHA resin bound peptides and intermediates

| Com- | | MF | |
|---|---|---|---|
| pound | | [M − resin − | MW |
| No. | Structure | C₁₇H₁₇NO₄ + H] | (Cal.) |
| 1 | 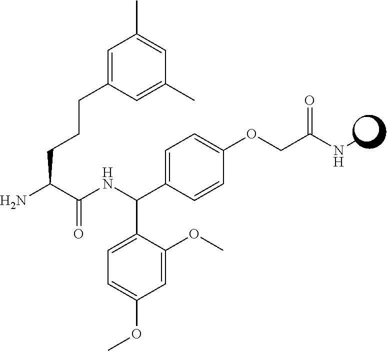 [SMILES: CNC(=O)COc1ccc(C(NC(=O)[C@@H](N)CCCc2cc(C)cc(C)c2)c2ccc(OC)cc2OC)cc1] | C₁₃H₂₀N₂O | 220.31 |
| 2 | 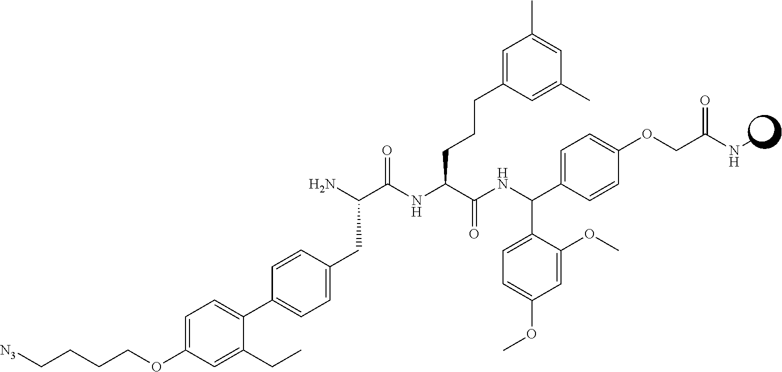 [SMILES: CCc1cc(OCCCCN=[N+]=[N-])ccc1-c1ccc(C[C@H](N)C(=O)N[C@@H](CCCc2cc(C)cc(C)c2)C(=O)NC(c2ccc(OCC(=O)NC)cc2)c2ccc(OC)cc2OC)cc1] | C₃₄H₄₄N₆O₃ | 584.75 |
| 3 | 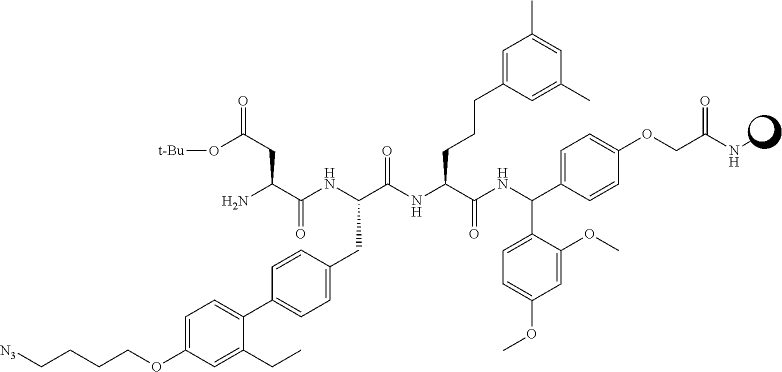 [SMILES: CCc1cc(OCCCCN=[N+]=[N-])ccc1-c1ccc(C[C@H](NC(=O)[C@@H](N)CC(=O)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₄₂H₅₇N₇O₆ | 755.95 |
| 4 | 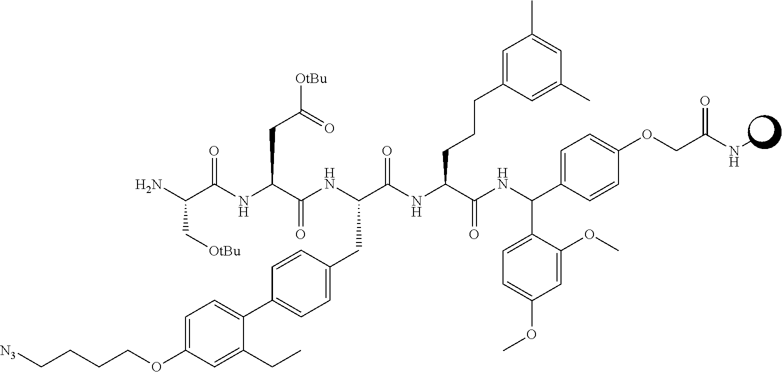 [SMILES: CCc1cc(OCCCCN=[N+]=[N-])ccc1-c1ccc(C[C@H](NC(=O)[C@H](CC(=O)OC(C)(C)C)NC(=O)[C@@H](N)COC(C)(C)C)C(=O)N[C@@H](CCCc2cc(C)cc(C)c2)C(=O)NC(c2ccc(OCC(=O)NC)cc2)c2ccc(OC)cc2OC)cc1] | C₄₉H₇₀N₈O₈ | 899.13 |
| 5 | 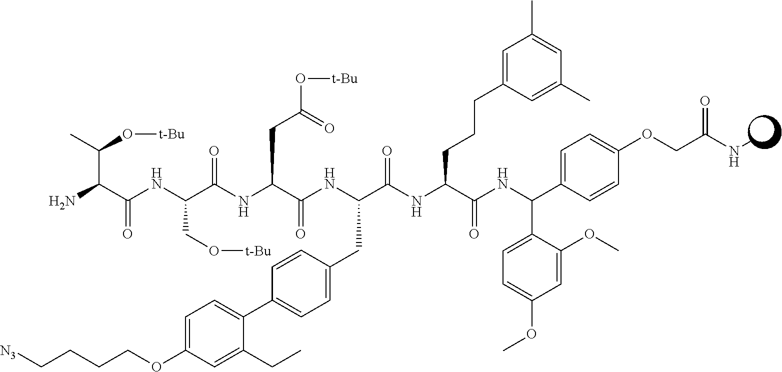 [SMILES: CCc1cc(OCCCCN=[N+]=[N-])ccc1-c1ccc(C[C@H](NC(=O)[C@H](CC(=O)OC(C)(C)C)NC(=O)[C@H](COC(C)(C)C)NC(=O)[C@@H](N)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₅₇H₈₅N₉O₁₀ | 1056.34 |
| 6 | 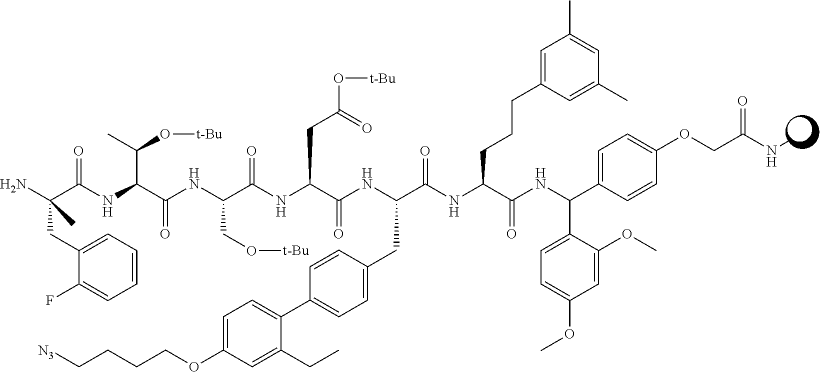 [SMILES: CCc1cc(OCCCCN=[N+]=[N-])ccc1-c1ccc(C[C@H](NC(=O)[C@H](CC(=O)OC(C)(C)C)NC(=O)[C@H](COC(C)(C)C)NC(=O)[C@@H](NC(=O)[C@@](C)(N)Cc2ccccc2F)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₆₇H₉₅FN₁₀O₁₁ | 1235.53 |
| 7 | 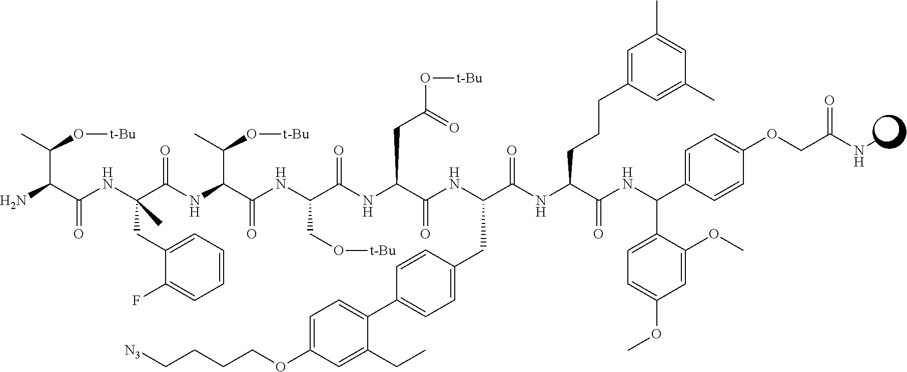 [SMILES: CCc1cc(OCCCCN=[N+]=[N-])ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](N)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₇₅H₁₁₀FN₁₁O₁₃ | 1392.74 |
| 8 | 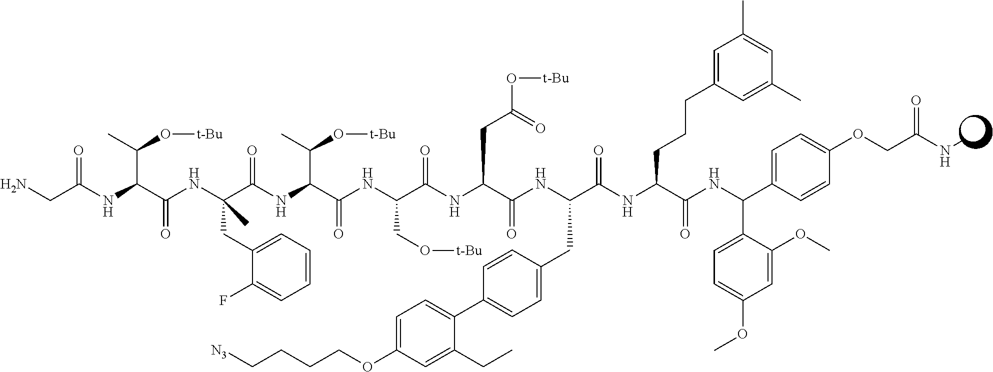 [SMILES: CCc1cc(OCCCCN=[N+]=[N-])ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CN)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₇₇H₁₁₃FN₁₂O₁₄ | 1449.79 |
| 9 | 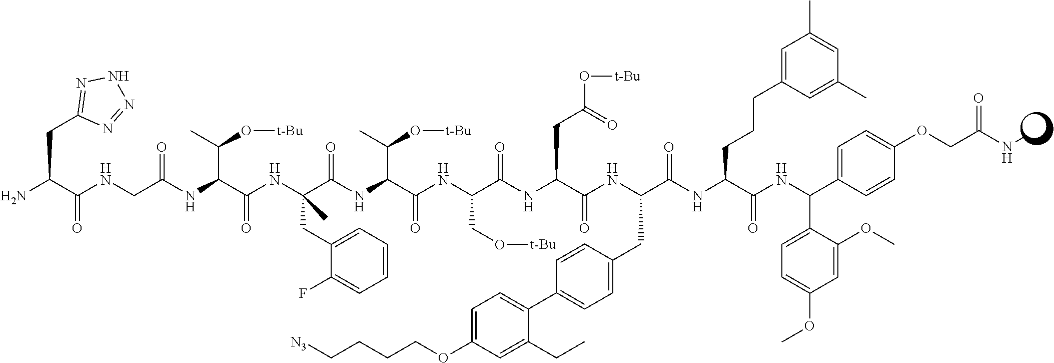 [SMILES: CCc1cc(OCCCCN=[N+]=[N-])ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₈₁H₁₁₈FN₁₇O₁₅ | 1588.91 |
| 10 | 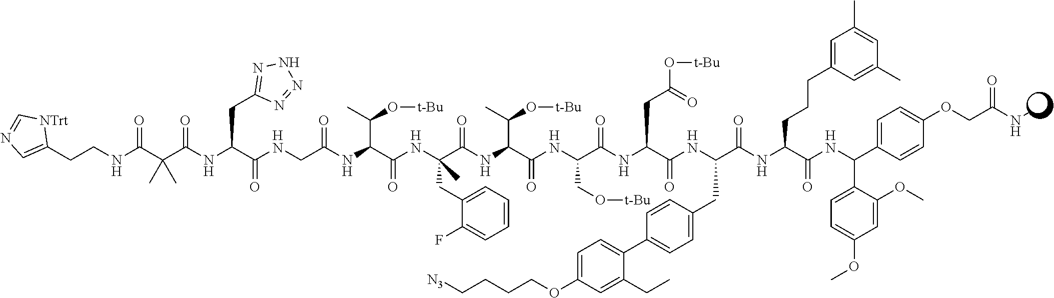 | C₁₁₀H₁₄₅FN₂₀O₁₇ | 2038.45 |
| 11 | 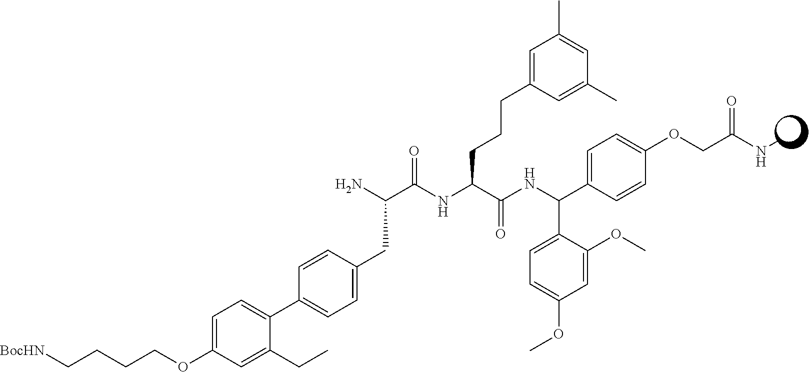 [SMILES: CCc1cc(OCCCCNC(=O)OC(C)(C)C)ccc1-c1ccc(C[C@H](N)C(=O)N[C@@H](CCCc2cc(C)cc(C)c2)C(=O)NC(c2ccc(OCC(=O)NC)cc2)c2ccc(OC)cc2OC)cc1] | C₃₉H₅₄N₄O₅ | 658.87 |
| 12 | 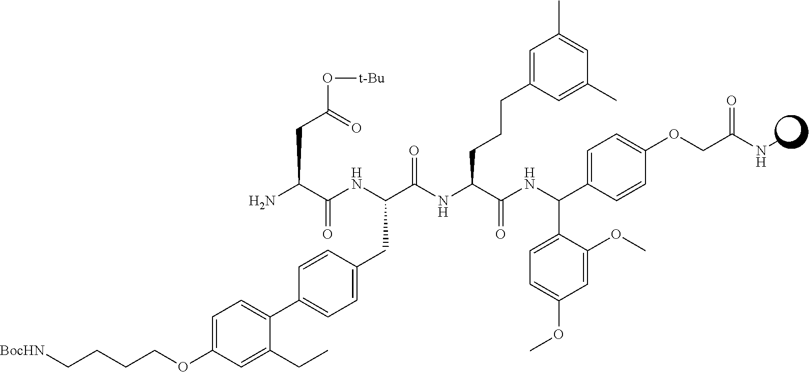 [SMILES: CCc1cc(OCCCCNC(=O)OC(C)(C)C)ccc1-c1ccc(C[C@H](NC(=O)[C@@H](N)CC(=O)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₄₇H₆₇N₅O₈ | 830.06 |
| 13 | 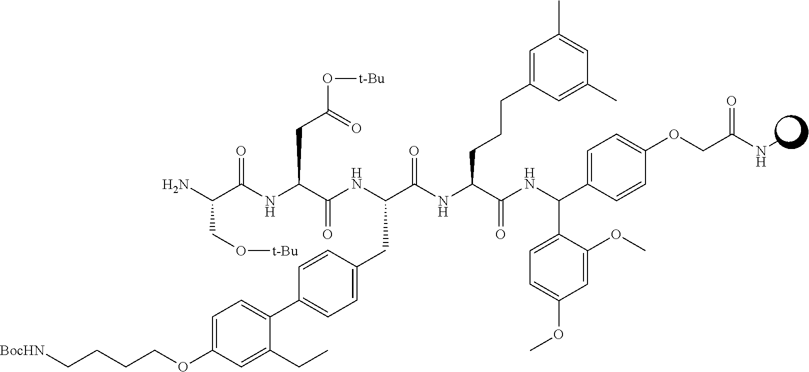 [SMILES: CCc1cc(OCCCCNC(=O)OC(C)(C)C)ccc1-c1ccc(C[C@H](NC(=O)[C@H](CC(=O)OC(C)(C)C)NC(=O)[C@@H](N)COC(C)(C)C)C(=O)N[C@@H](CCCc2cc(C)cc(C)c2)C(=O)NC(c2ccc(OCC(=O)NC)cc2)c2ccc(OC)cc2OC)cc1] | C₅₄H₈₀N₆O₁₀ | 973.24 |
| 14 | 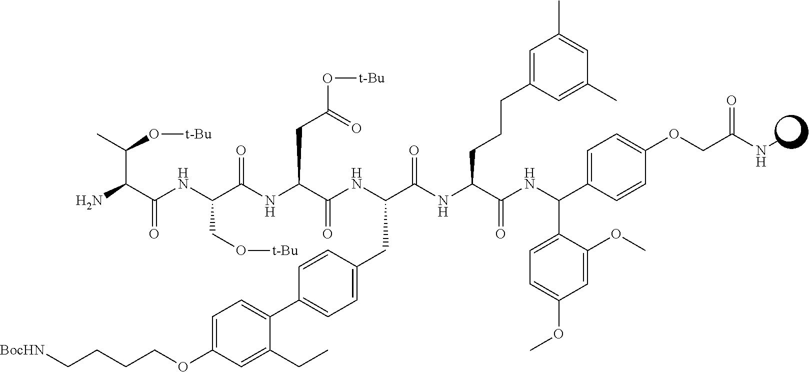 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](N)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₆₂H₉₅N₇O₁₂ | 1130.45 |
| 15 | 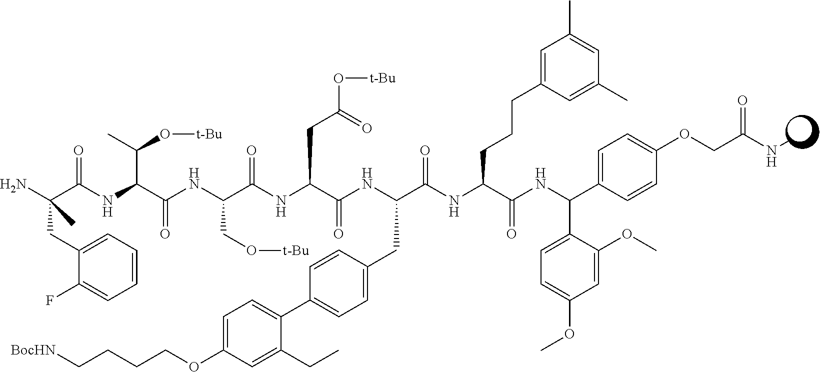 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@@](C)(N)Cc2ccccc2F)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₇₂H₁₀₅FN₈O₁₃ | 1309.64 |
| 16 | 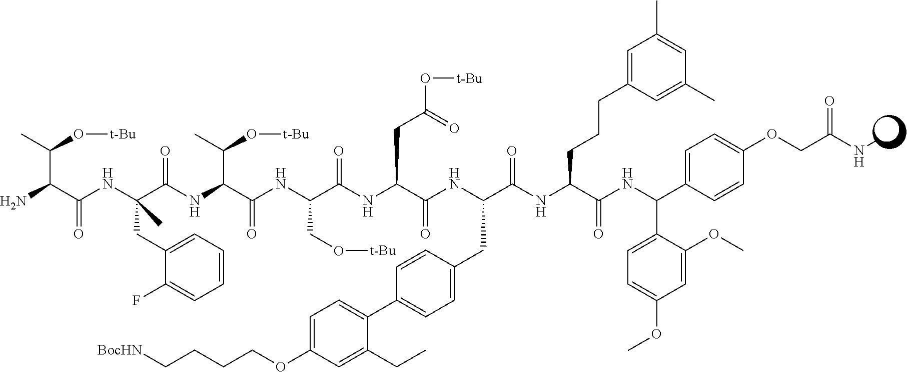 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](N)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₈₀H₁₂₀FN₉O₁₅ | 1466.85 |
| 17 | 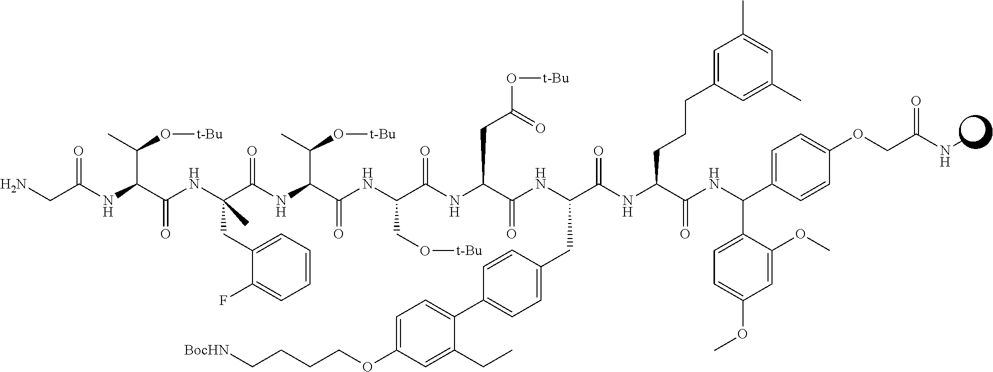 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CN)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₈₂H₁₂₃FN₁₀O₁₆ | 1523.91 |
| 18 | 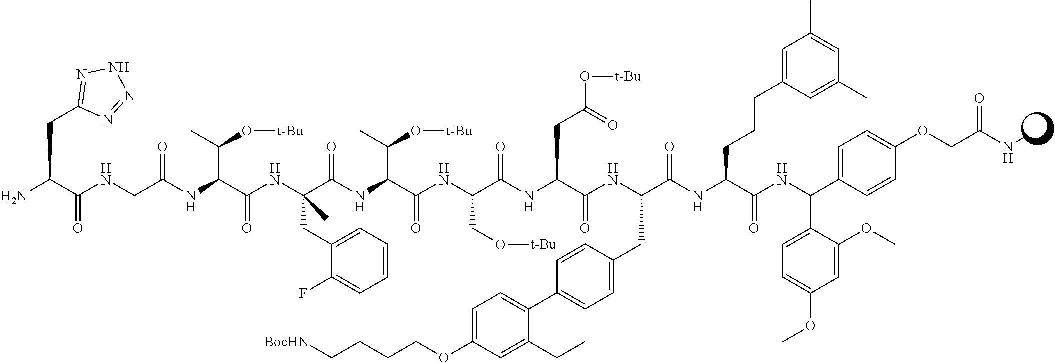 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₈₆H₁₂₈FN₁₅O₁₇ | 1663.02 |
| 19 | 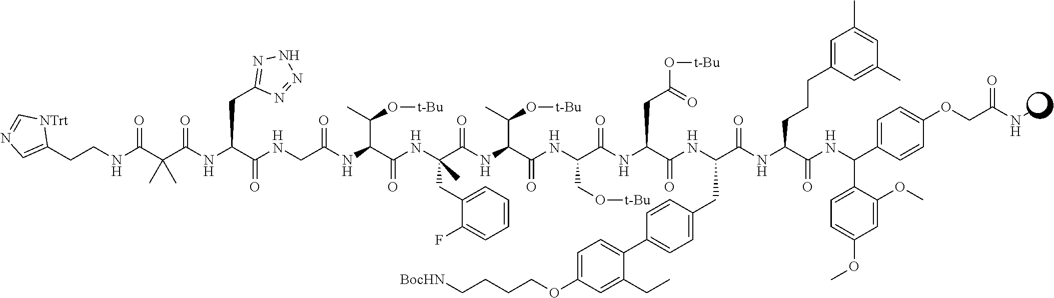 | C₁₁₅H₁₅₅FN₁₈O₁₉ | 2112.56 |
| 20 | 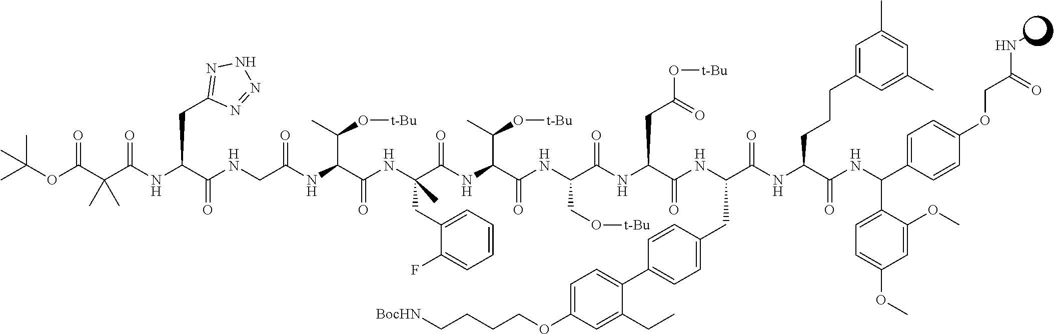 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)OC(C)(C)C)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₉₅H₁₄₂FN₁₅O₂₀ | 1833.23 |
| 21 | 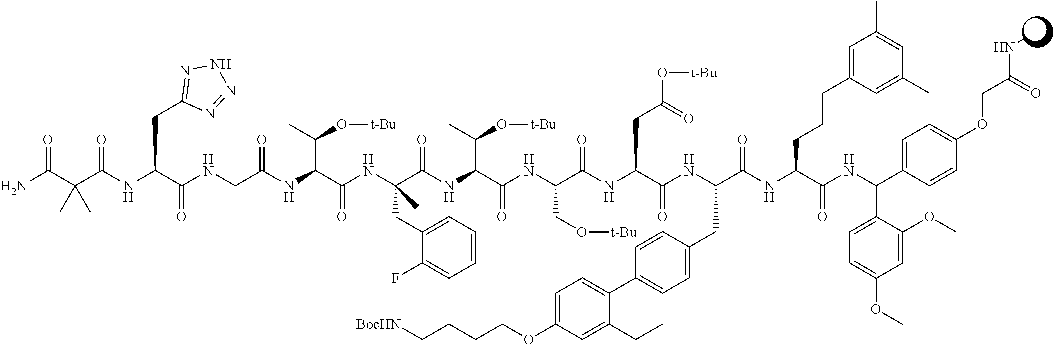 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₉₁H₁₃₅FN₁₆O₁₉ | 1776.14 |
| 22 | 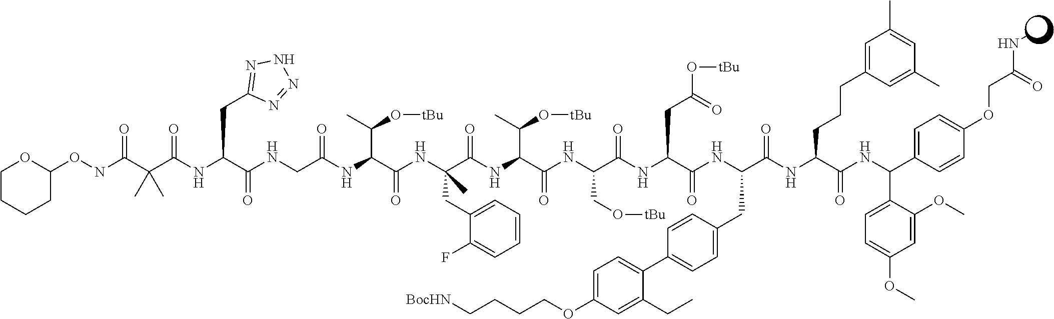 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NOC2CCCCO2)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₉₆H₁₄₂FN₁₆O₂₁ | 1875.25 |
| 23 | 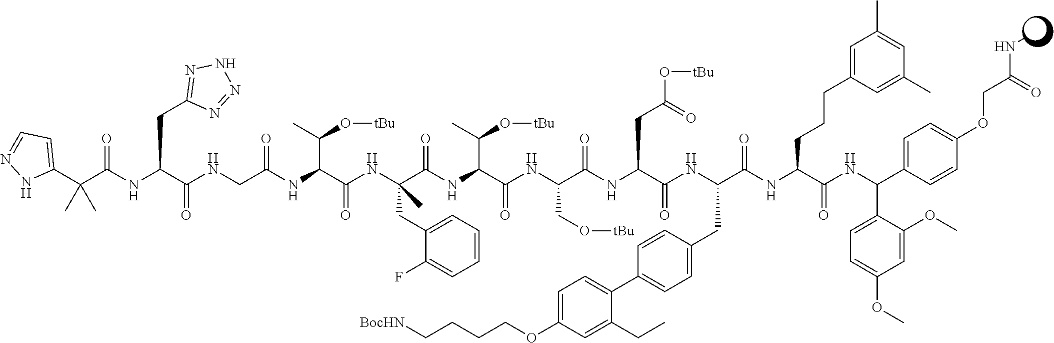 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)c2ccn[nH]2)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₉₃H₁₃₆FN₁₇O₁₈ | 1799.18 |
| 24 | 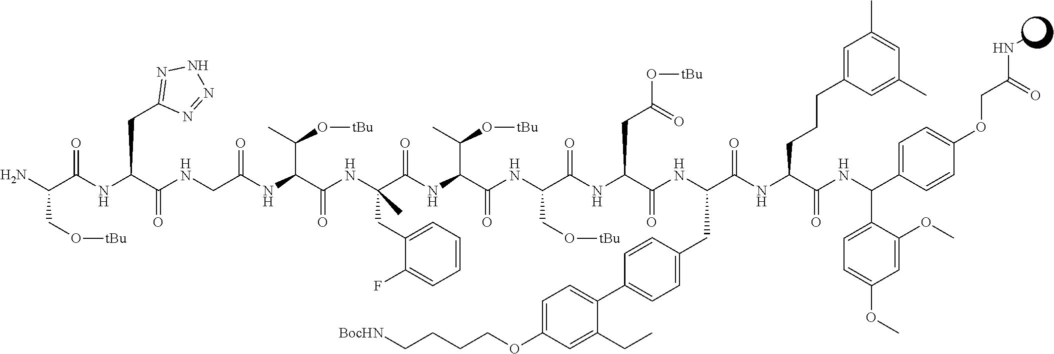 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)[C@@H](N)COC(C)(C)C)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₉₃H₁₄₁FN₁₆O₁₉ | 1806.21 |
| 25 | 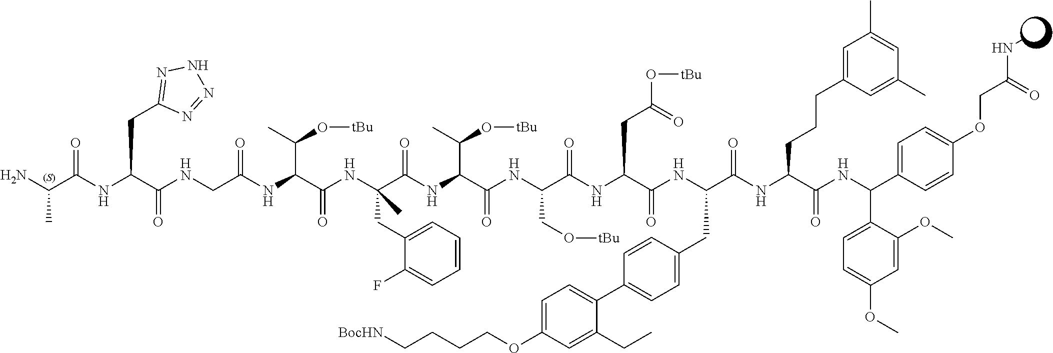 | C₈₉H₁₃₃FN₁₆O₁₈ | 1734.10 |
| 26 | 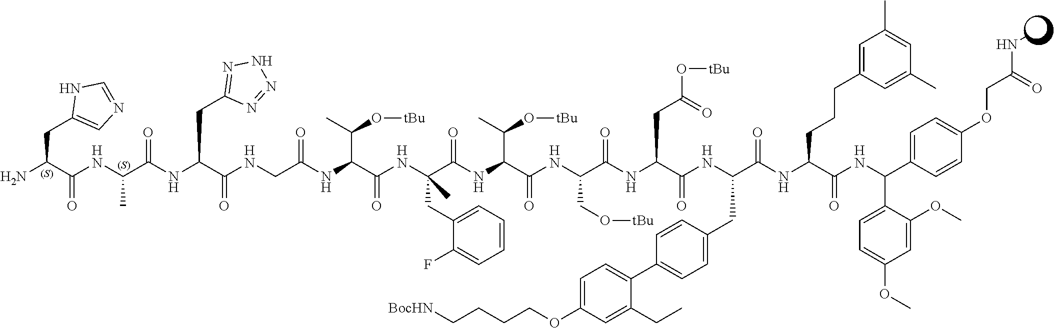 | C₉₅H₁₄₀FN₁₉O₁₉ | 1871.24 |
| 27 | 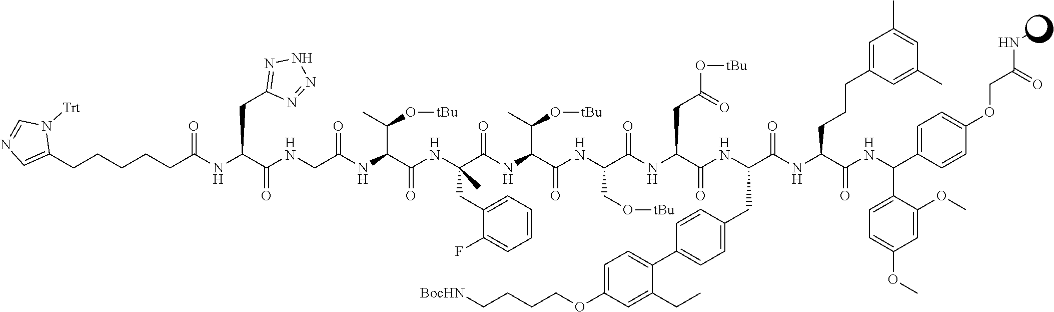 | C₁₁₄H₁₅₄FN₁₇O₁₈ | 2069.54 |
| 28 | 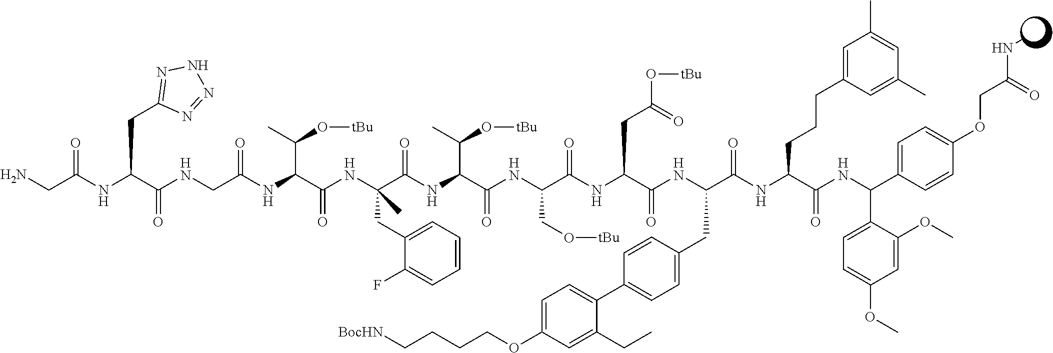 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)CN)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₈₈H₁₃₁FN₁₆O₁₈ | 1720.08 |
| 29 | 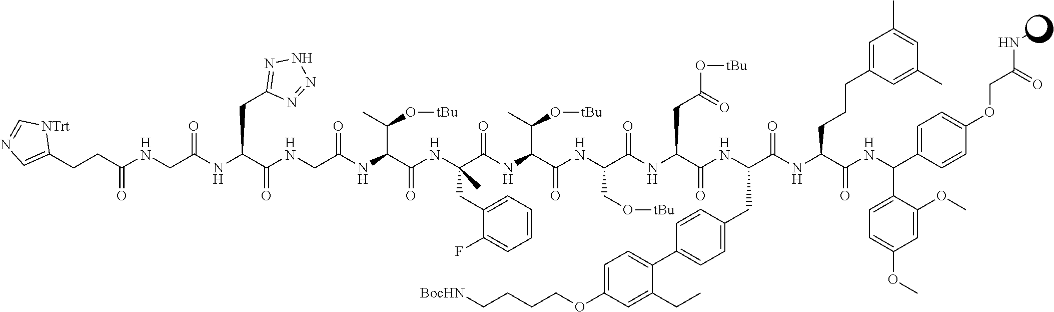 | C₁₁₃H₁₅₁FN₁₈O₁₉ | 2084.52 |
| 30 | 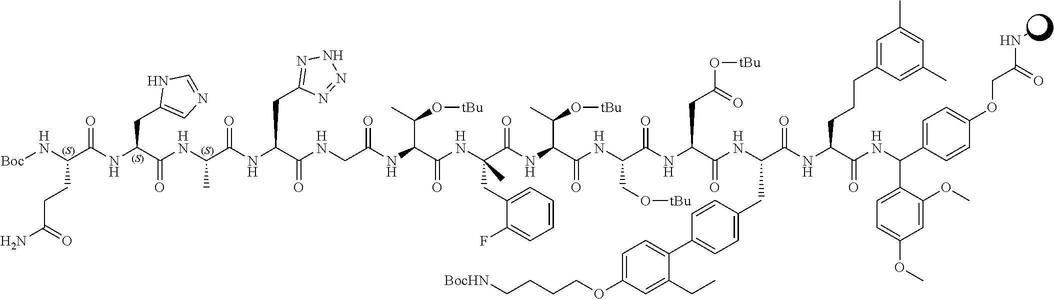 | C₁₀₅H₁₅₆FN₂₁O₂₃ | 2099.49 |
| 31 | 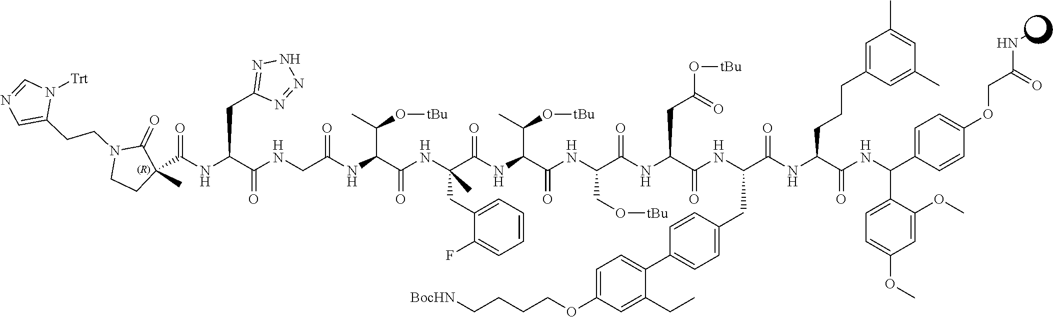 | C₁₁₆H₁₅₅FN₁₈O₁₉ | 2124.58 |
| 32 | 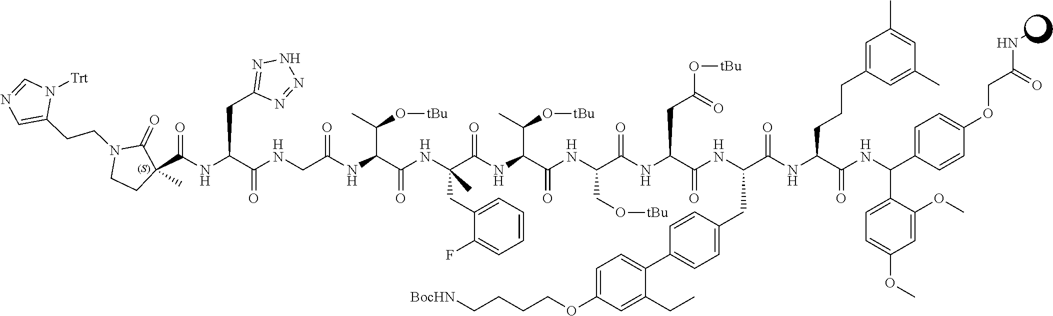 | C₁₁₆H₁₅₅FN₁₈O₁₉ | 2124.58 |
| 33 | 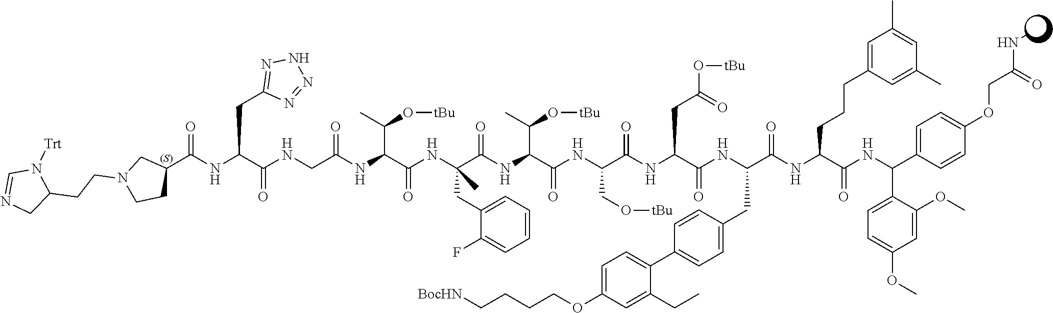 | C₁₁₅H₁₅₅FN₁₈O₁₈ | 2096.57 |
| 34 | 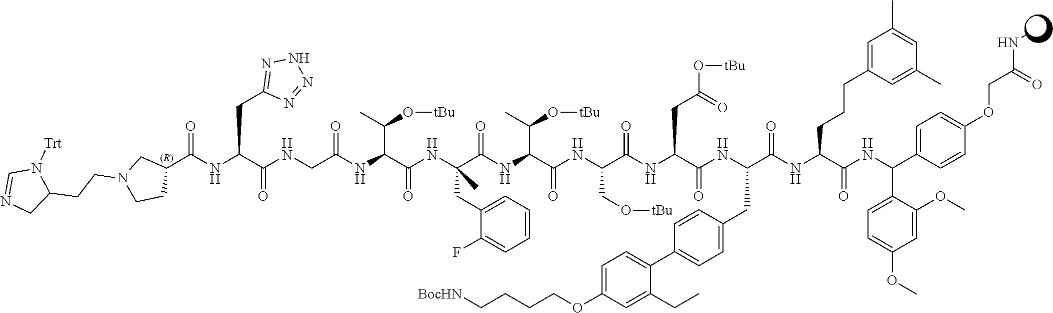 | C₁₁₅H₁₅₅FN₁₈O₁₈ | 2096.57 |
| 35 | 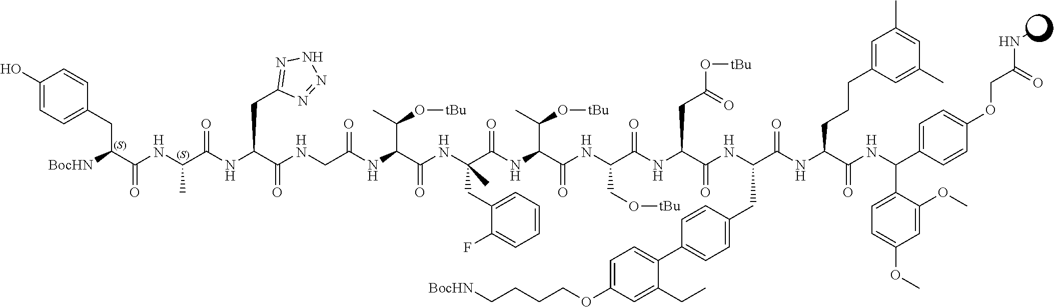 | C₁₀₃H₁₅₀FN₁₇O₂₂ | 1997.39 |
| 36 | 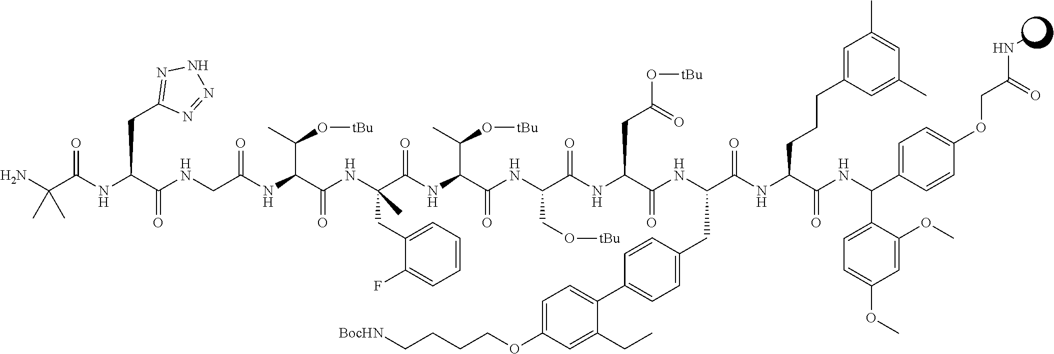 [SMILES: CCc1cc(OCCCCNC(=O)OC(C)(C)C)ccc1-c1ccc(C[C@H](NC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)N)[C@@H](C)OC(C)(C)C)[C@@H](C)OC(C)(C)C)C(=O)N[C@@H](CCCc2cc(C)cc(C)c2)C(=O)NC(c2ccc(OCC(=O)NC)cc2)c2ccc(OC)cc2OC)cc1] | C₉₀H₁₃₅FN₁₆O₁₈ | 1748.13 |
| 37 | 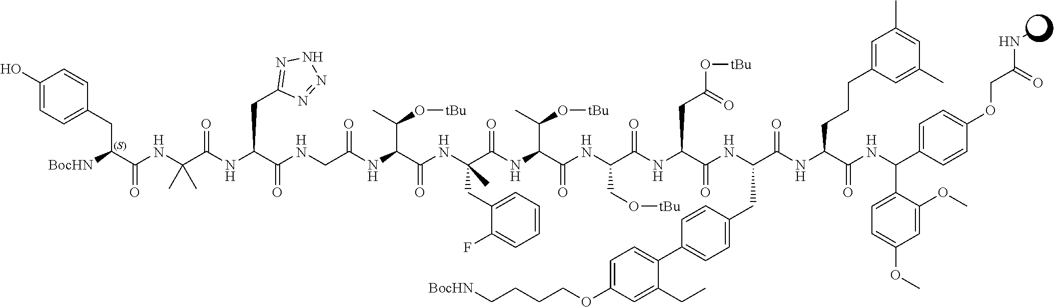 | C₁₀₄H₁₅₂FN₁₇O₂₂ | 2011.42 |
| 38 | 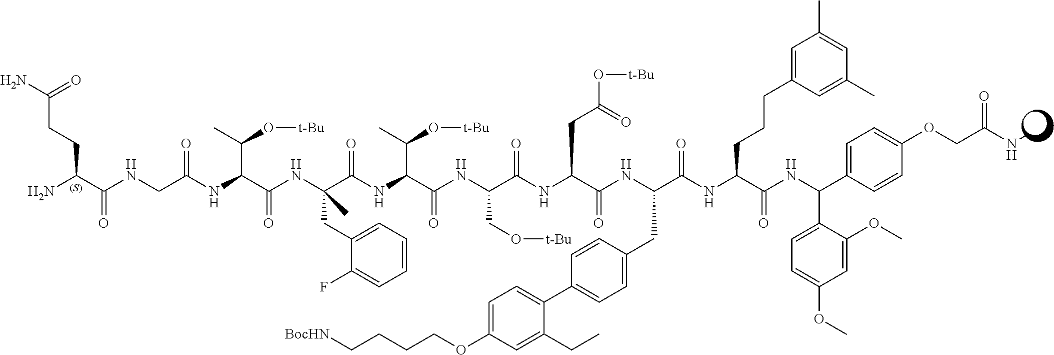 | C₈₇H₁₃₁FN₁₂O₁₈ | 1652.04 |
| 39 | 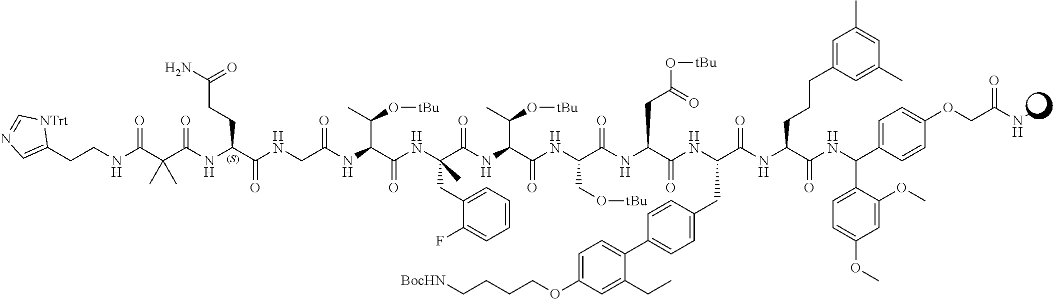 | C₁₁₆H₁₅₈FN₁₅O₂₀ | 2101.58 |
| 40 | 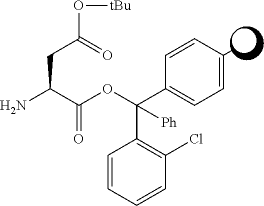 [SMILES: Cc1ccc(C(OC(=O)[C@@H](N)CC(=O)OC(C)(C)C)(c2ccccc2)c2ccccc2Cl)cc1] | [M − resin − CITrt + H] C₈H₁₅NO₄ | 189.21 |
| 41 | 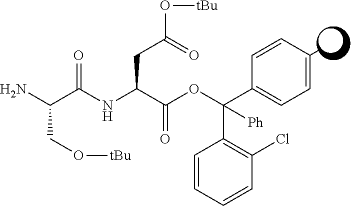 [SMILES: Cc1ccc(C(OC(=O)[C@H](CC(=O)OC(C)(C)C)NC(=O)[C@@H](N)COC(C)(C)C)(c2ccccc2)c2ccccc2Cl)cc1] | [M − resin − CITrt + H] C₁₅H₂₈N₂O₆ | 332.39 |
| 42 | 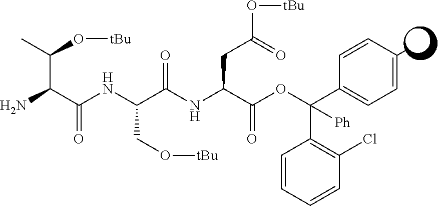 [SMILES: Cc1ccc(C(OC(=O)[C@H](CC(=O)OC(C)(C)C)NC(=O)[C@H](COC(C)(C)C)NC(=O)[C@@H](N)[C@@H](C)OC(C)(C)C)(c2ccccc2)c2ccccc2Cl)cc1] | [M − resin − CITrt + H] C₂₃H₄₃N₃O₈ | 489.60 |
| 43 | 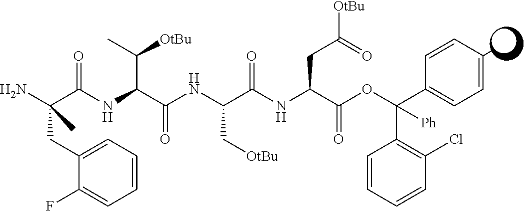 [SMILES: Cc1ccc(C(OC(=O)[C@H](CC(=O)OC(C)(C)C)NC(=O)[C@H](COC(C)(C)C)NC(=O)[C@@H](NC(=O)[C@@](C)(N)Cc2ccccc2F)[C@@H](C)OC(C)(C)C)(c2ccccc2)c2ccccc2Cl)cc1] | [M − resin − CITrt + H] C₃₃H₅₃FN₄O₉ | 668.79 |
| 44 | 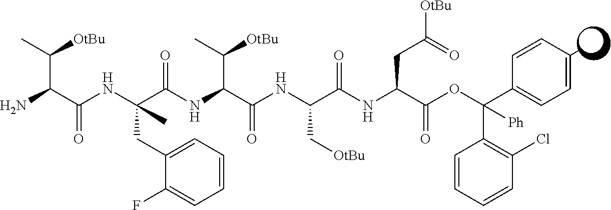 [SMILES: Cc1ccc(C(OC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](N)[C@@H](C)OC(C)(C)C)[C@@H](C)OC(C)(C)C)(c2ccccc2)c2ccccc2Cl)cc1] | [M − resin − CITrt + H] C₄₁H₆₈FN₅O₁₁ | 826.00 |
| 45 | 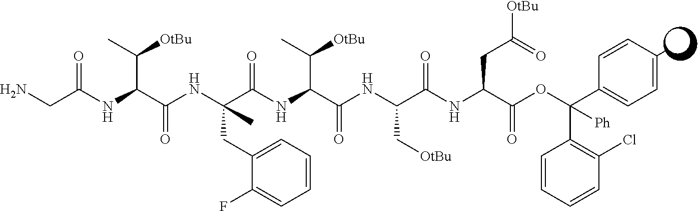 [SMILES: Cc1ccc(C(OC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CN)[C@@H](C)OC(C)(C)C)[C@@H](C)OC(C)(C)C)(c2ccccc2)c2ccccc2Cl)cc1] | [M − resin − CITrt + H] C₄₃H₇₁FN₆O₁₂ | 883.06 |
| 46 | 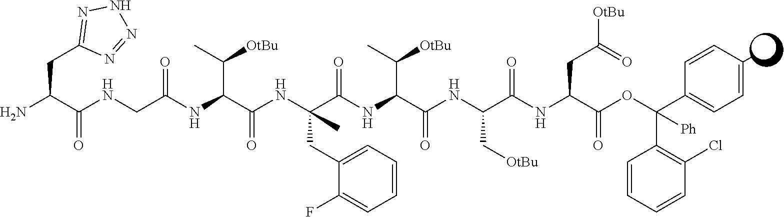 [SMILES: Cc1ccc(C(OC(=O)[C@H](CC(=O)OC(C)(C)C)NC(=O)[C@H](COC(C)(C)C)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)OC(C)(C)C)[C@@H](C)OC(C)(C)C)(c2ccccc2)c2ccccc2Cl)cc1] | [M − resin − CITrt + H] C₄₇H₇₆FN₁₁O₁₃ | 1022.17 |
| 47 | 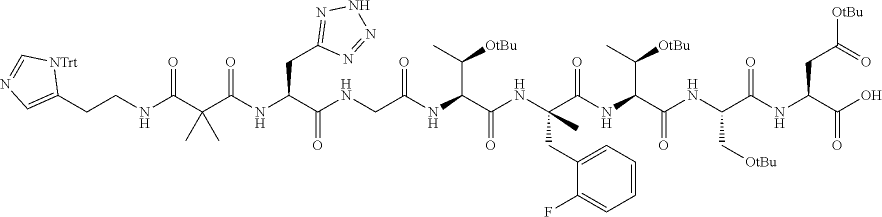 | C₇₆H₁₀₃FN₁₄O₁₅ | 1471.71 |
| 48 | 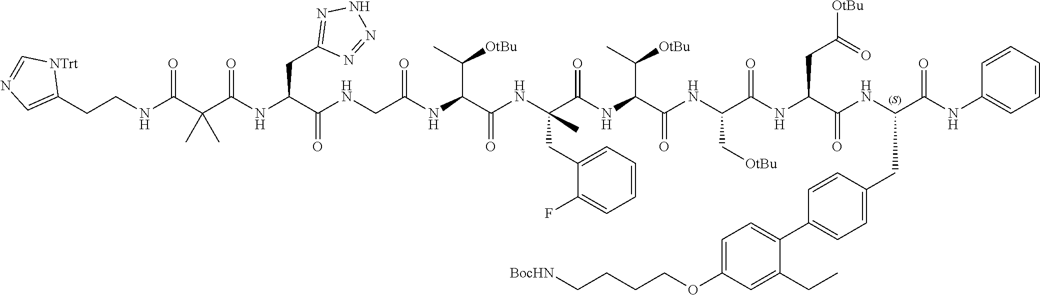 | C₁₀₈H₁₄₂FN₁₇O₁₈ | 1985.38 |
| 49 | 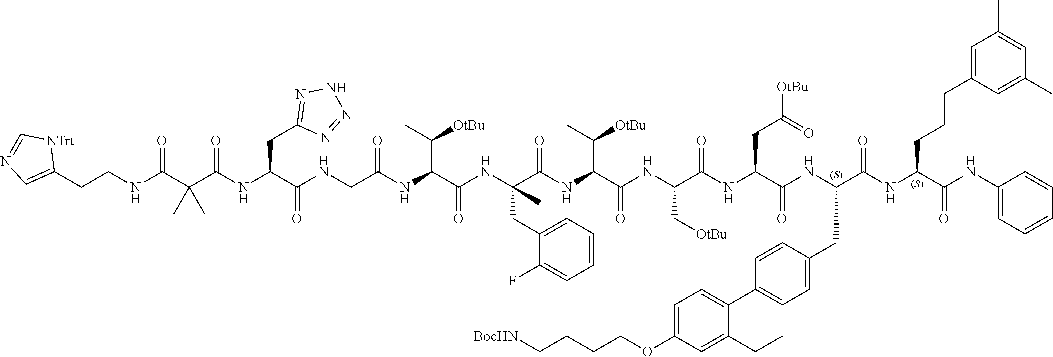 | C₁₂₁H₁₅₉FN₁₈O₁₉ | 2188.66 |
| 50 | 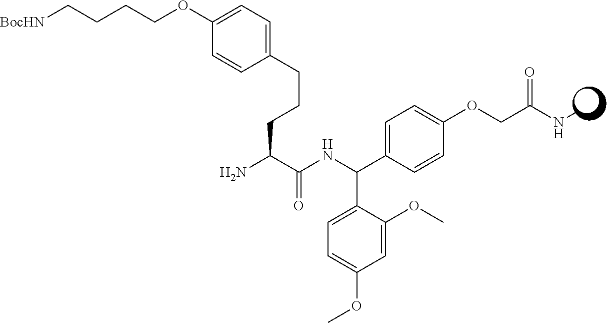 [SMILES: CNC(=O)COc1ccc(C(NC(=O)[C@@H](N)CCCc2ccc(OCCCCNC(=O)OC(C)(C)C)cc2)c2ccc(OC)cc2OC)cc1] | C₂₀H₃₃N₃O₄ | 379.49 |
| 51 | 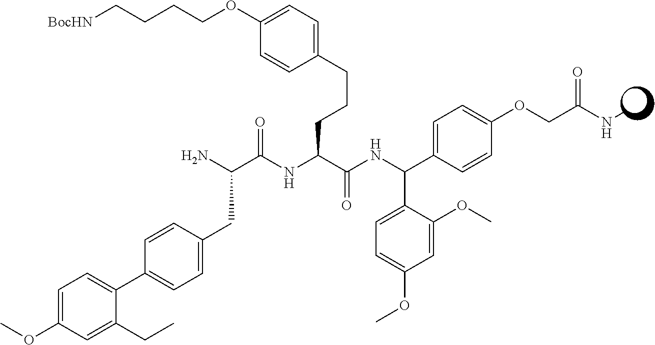 | C₃₈H₅₂N₄O₆ | 660.3887 |
| 52 | 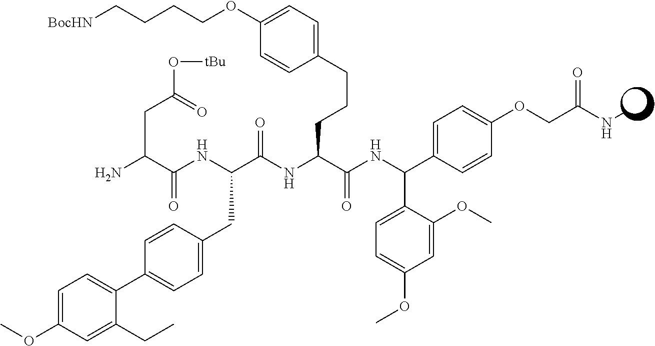 | C₄₆H₆₅N₅O₉ | 831.4782 |
| 53 | 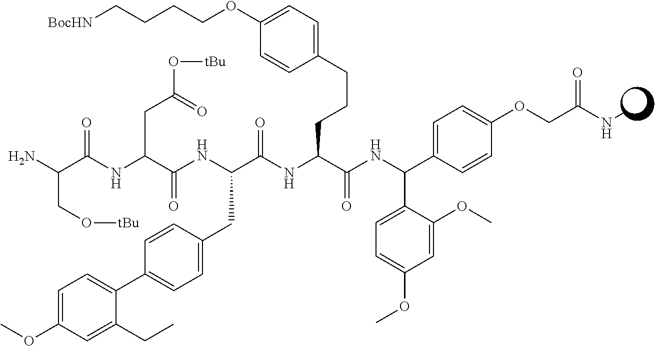 | C₅₃H₇₈N₆O₁₁ | 974.5729 |
| 54 | 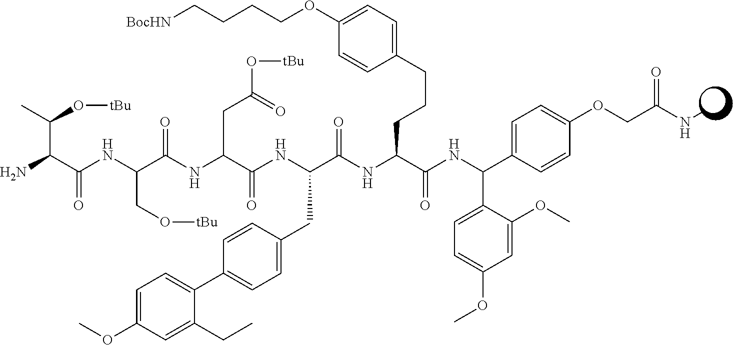 [SMILES: CCc1cc(OC)ccc1-c1ccc(C[C@H](NC(=O)C(CC(=O)OC(C)(C)C)NC(=O)C(COC(C)(C)C)NC(=O)[C@@H](N)[C@@H](C)OC(C)(C)C)C(=O)N[C@@H](CCCc2ccc(OCCCCNC(=O)OC(C)(C)C)cc2)C(=O)NC(c2ccc(OCC(=O)NC)cc2)c2ccc(OC)cc2OC)cc1] | C₆₁H₉₃N₇O₁₃ | 1131.68 |
| 55 | 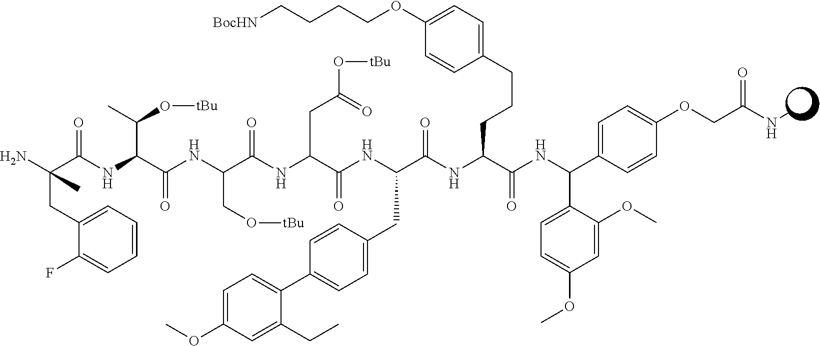 [SMILES: CCc1cc(OC)ccc1-c1ccc(C[C@H](NC(=O)C(CC(=O)OC(C)(C)C)NC(=O)C(COC(C)(C)C)NC(=O)[C@@H](NC(=O)[C@@](C)(N)Cc2ccccc2F)[C@@H](C)OC(C)(C)C)C(=O)N[C@@H](CCCc2ccc(OCCCCNC(=O)OC(C)(C)C)cc2)C(=O)NC(c2ccc(OCC(=O)NC)cc2)c2ccc(OC)cc2OC)cc1] | C₇₁H₁₀₃FN₈O₁₄ | 1310.76 |
| 56 | 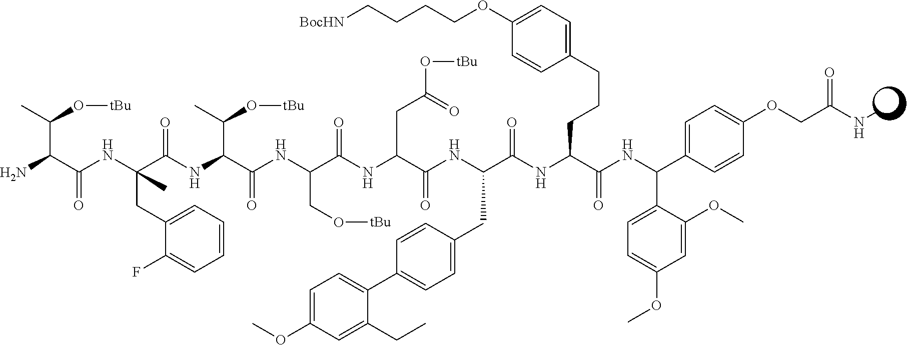 [SMILES: CCc1cc(OC)ccc1-c1ccc(C[C@H](NC(=O)C(CC(=O)OC(C)(C)C)NC(=O)C(COC(C)(C)C)NC(=O)[C@@H](NC(=O)[C@](C)(Cc2ccccc2F)NC(=O)[C@@H](N)[C@@H](C)OC(C)(C)C)[C@@H](C)OC(C)(C)C)C(=O)N[C@@H](CCCc2ccc(OCCCCNC(=O)OC(C)(C)C)cc2)C(=O)NC(c2ccc(OCC(=O)NC)cc2)c2ccc(OC)cc2OC)cc1] | C₇₉H₁₁₈FN₉O₁₆ | 1467.868 |
| 57 | 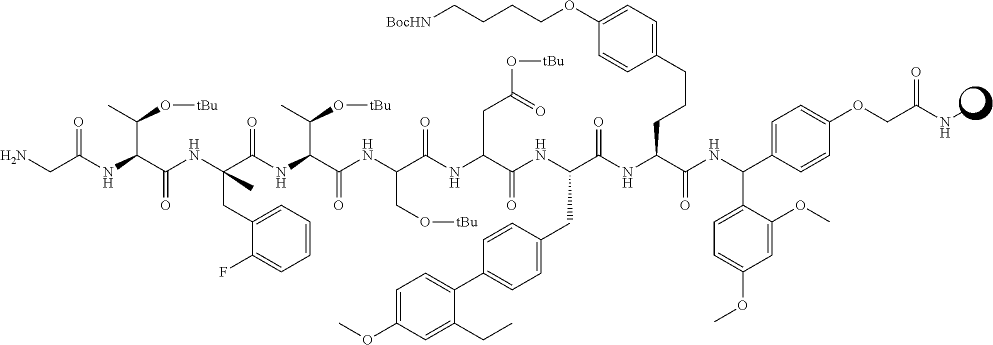 [SMILES: CCc1cc(OC)ccc1-c1ccc(C[C@H](NC(=O)C(CC(=O)OC(C)(C)C)NC(=O)C(COC(C)(C)C)NC(=O)[C@@H](NC(=O)[C@](C)(Cc2ccccc2F)NC(=O)[C@@H](NC(=O)CN)[C@@H](C)OC(C)(C)C)[C@@H](C)OC(C)(C)C)C(=O)N[C@@H](CCCc2ccc(OCCCCNC(=O)OC(C)(C)C)cc2)C(=O)NC(c2ccc(OCC(=O)NC)cc2)c2ccc(OC)cc2OC)cc1] | C₈₁H₁₂₁FN₁₀O₁₇ | 1524.8895 |
| 58 | 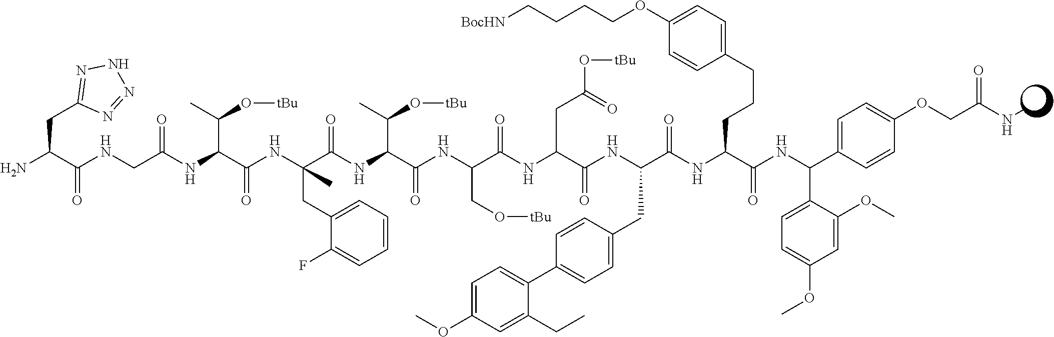 [SMILES: CCc1cc(OC)ccc1-c1ccc(C[C@H](NC(=O)C(CC(=O)OC(C)(C)C)NC(=O)C(COC(C)(C)C)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)OC(C)(C)C)[C@@H](C)OC(C)(C)C)C(=O)N[C@@H](CCCc2ccc(OCCCCNC(=O)OC(C)(C)C)cc2)C(=O)NC(c2ccc(OCC(=O)NC)cc2)c2ccc(OC)cc2OC)cc1] | C₈₅H₁₂₆FN₁₅O₁₈ | 1663.9389 |
| 59 | 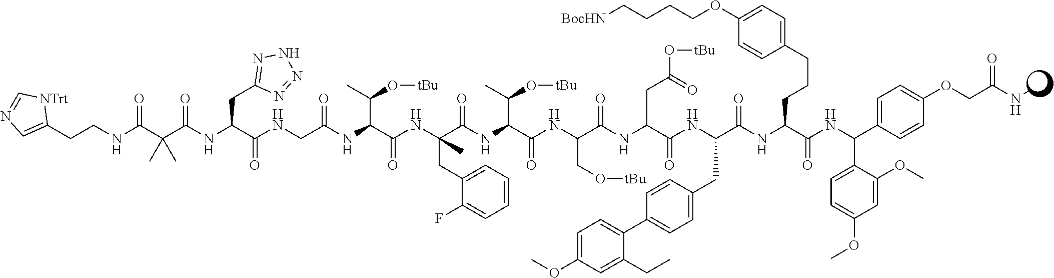 | C₁₁₄H₁₅₃FN₁₈O₂₀ | 2113.149 |

The Rink amide MBHA resin bound peptides and intermediates (1˜59) were analyzed by LC-MS after cleavage from the Resin. Table 2B, below, summarizes these intermediates.

TABLE 2B
Intermediates Cleaved from MBHA Resin (Table discloses SEQ ID NOS 570-587, 488, 588-590, 480, 591-599 and 487, respectively, in order of appearance)

| | | | MW | |
|---|---|---|---|---|
| No. | Structure | MF | (Cal.) | MS (m/z) |
| 1 | 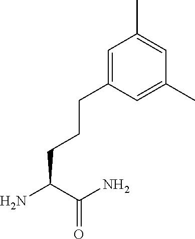 [SMILES: Cc1cc(C)cc(CCC[C@H](N)C(N)=O)c1] | C₁₃H₂₀N₂O | 220.3 | 221.4 [M + H]⁺ |
| 2 | 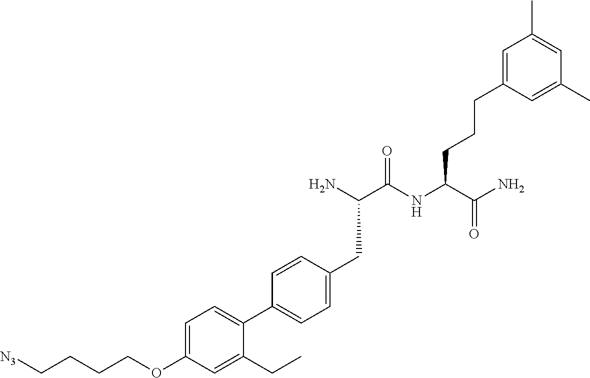 [SMILES: CCc1cc(OCCCCN=[N+]=[N-])ccc1-c1ccc(C[C@H](N)C(=O)N[C@@H](CCCc2cc(C)cc(C)c2)C(N)=O)cc1] | C₃₄H₄₄N₆O₃ | 584.8 | 585.3 [M + H]⁺ 607.30 [M + Na]⁺ |
| 3 | 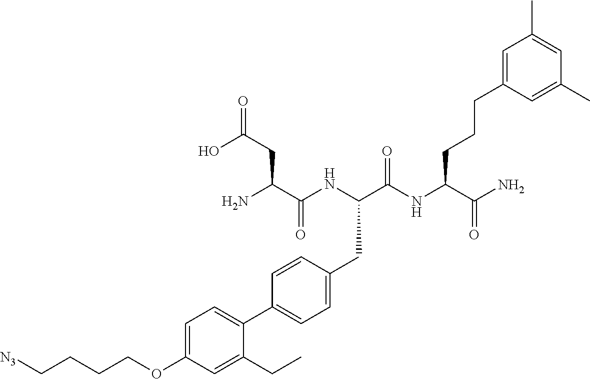 [SMILES: CCc1cc(OCCCCN=[N+]=[N-])ccc1-c1ccc(C[C@H](NC(=O)[C@@H](N)CC(=O)O)C(=O)N[C@@H](CCCc2cc(C)cc(C)c2)C(N)=O)cc1] | C₃₈H₄₉N₇O₆ | 699.4 | 700.4 [M + H]⁺ |
| 4 | 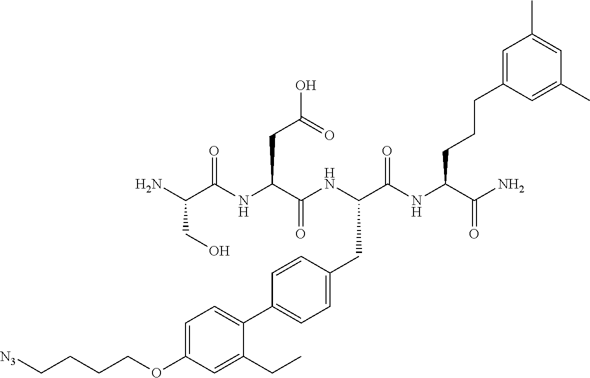 [SMILES: CCc1cc(OCCCCN=[N+]=[N-])ccc1-c1ccc(C[C@H](NC(=O)[C@H](CC(=O)O)NC(=O)[C@@H](N)CO)C(=O)N[C@@H](CCCc2cc(C)cc(C)c2)C(N)=O)cc1] | C₄₁H₅₄N₈O₈ | 786.4 | 787.4 [M + H]* |
| 5 | 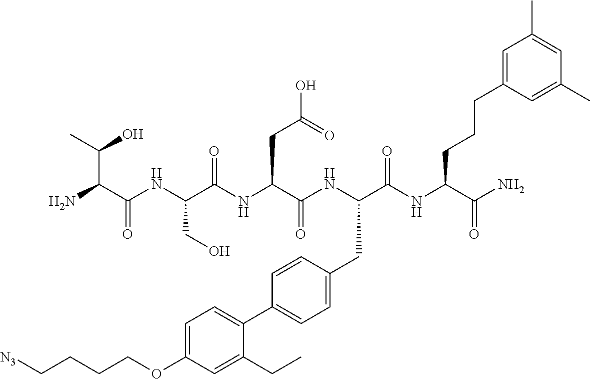 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](N)[C@@H](C)O)C(=O)N[C@@H](CCCc2cc(C)cc(C)c2)C(N)=O)cc1] | C₄₅H₆₁N₉O₁₀ | 887.5 | 888.5 [M + H]⁺ |
| 6 | 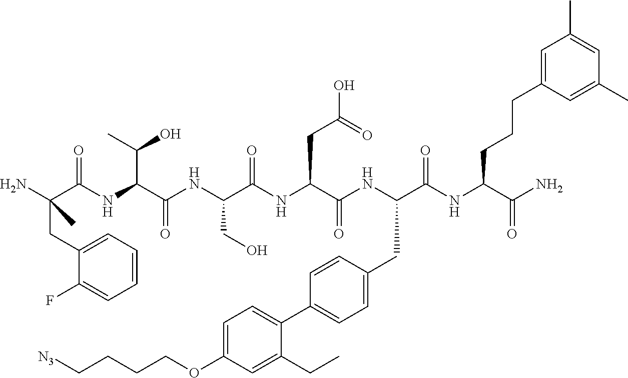 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@@](C)(N)Cc2ccccc2F)[C@@H](C)O)C(=O)N[C@@H](CCCc2cc(C)cc(C)c2)C(N)=O)cc1] | C₅₅H₇₁FN₁₀O₁₁ | 1066.7 | 1067.7 [M + H]⁺ |
| 7 | 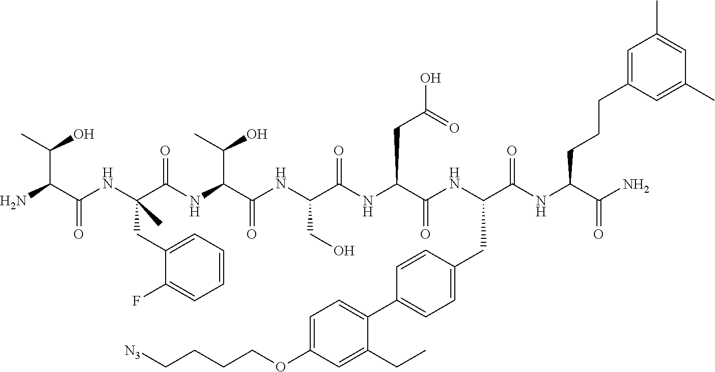 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](N)[C@@H](C)O)[C@@H](C)O)C(=O)N[C@@H](CCCc2cc(C)cc(C)c2)C(N)=O)cc1] | C₅₉H₇₈FN₁₁O₁₃ | 1167.6 | 1168.2 [M + H]⁺ |
| 8 | 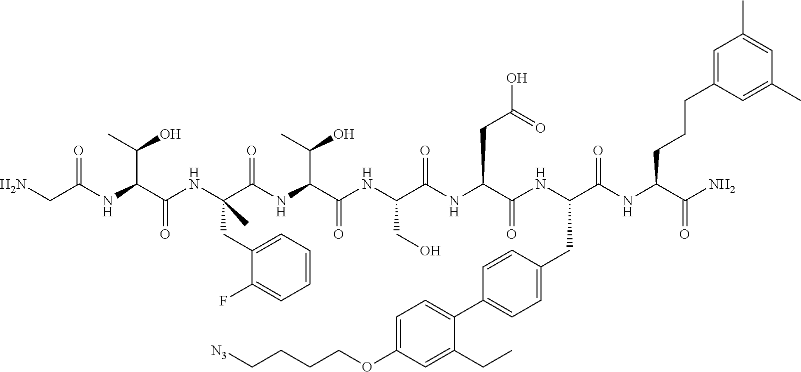 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CN)[C@@H](C)O)[C@@H](C)O)C(=O)N[C@@H](CCCc2cc(C)cc(C)c2)C(N)=O)cc1] | C₆₁H₈₁FN₁₂O₁₄ | 1224.6 | 1225.9 [M + H]⁺ |
| 9 | 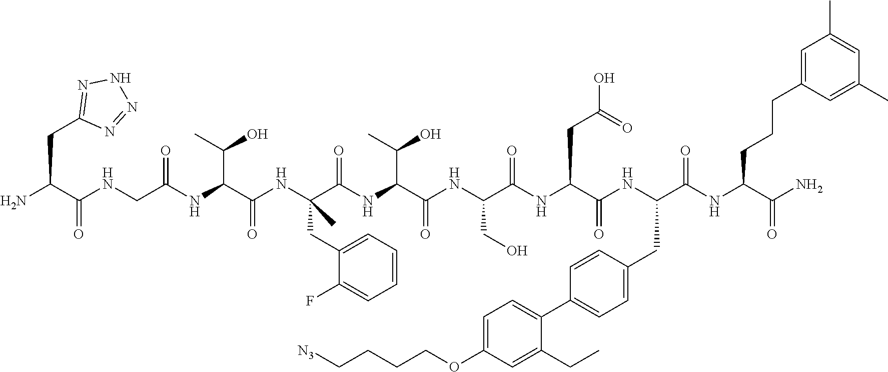 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(N)=O)cc1] | C₆₅H₈₆FN₁₇O₁₅ | 1363.6 | 1364.9 [M + H]⁺ |
| 10 | 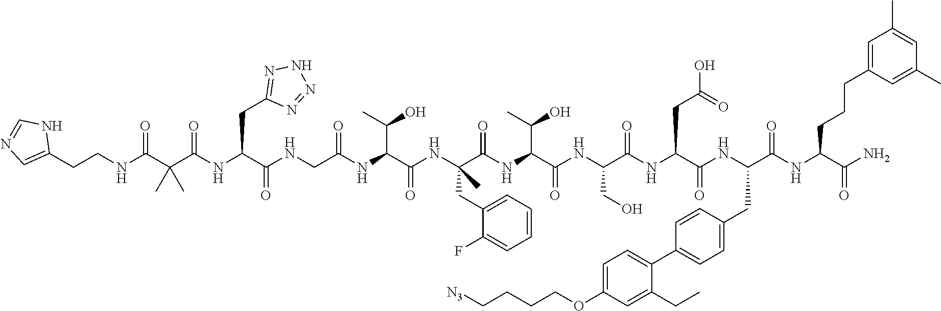 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] | C₇₅H₉₉FN₂₀O₁₇ | 1570.7 | 787.1 [M + 2H]⁺ |
| 11 | 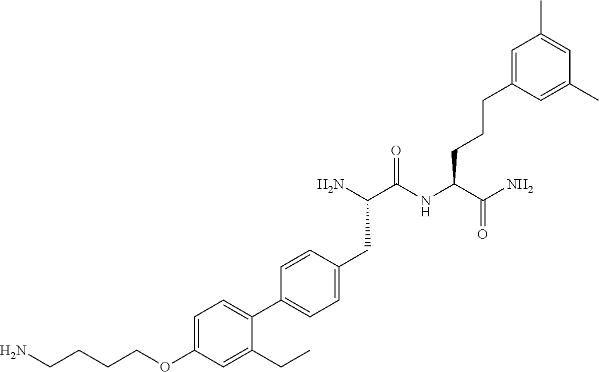 [SMILES: CCc1cc(OCCCCN)ccc1-c1ccc(C[C@H](N)C(=O)N[C@@H](CCCc2cc(C)cc(C)c2)C(N)=O)cc1] | C₃₄H₄₆N₄O₃ | 558.4 | 559.4 [M + H]⁺ |
| 12 | 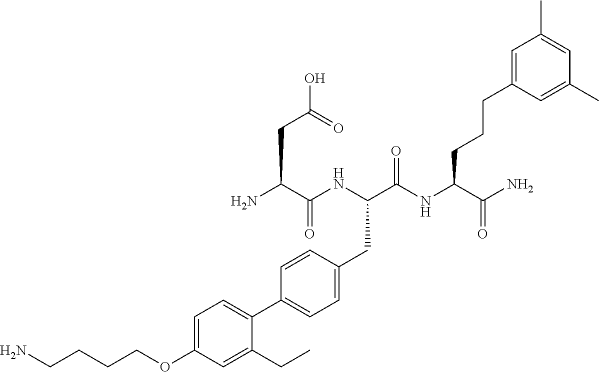 [SMILES: CCc1cc(OCCCCN)ccc1-c1ccc(C[C@H](NC(=O)[C@@H](N)CC(=O)O)C(=O)N[C@@H](CCCc2cc(C)cc(C)c2)C(N)=O)cc1] | C₃₈H₅₁N₅O₆ | 673.4 | 674.2 [M + H]⁺ |
| 13 | 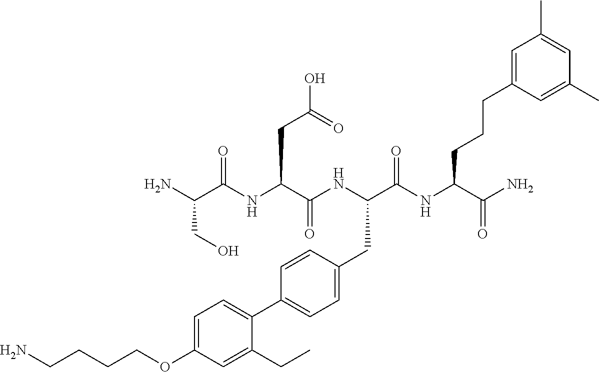 [SMILES: CCc1cc(OCCCCN)ccc1-c1ccc(C[C@H](NC(=O)[C@H](CC(=O)O)NC(=O)[C@@H](N)CO)C(=O)N[C@@H](CCCc2cc(C)cc(C)c2)C(N)=O)cc1] | C₄₁H₅₆N₆O₈ | 760.4 | 871.4 [M + H]+ 381.3 [M + 2H]²⁺ |
| 14 | 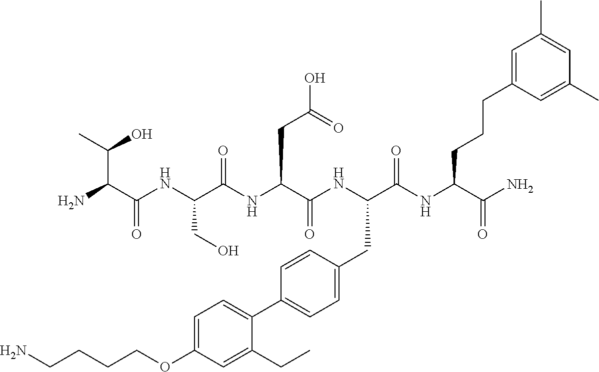 [SMILES: CCc1cc(OCCCCN)ccc1-c1ccc(C[C@H](NC(=O)[C@H](CC(=O)O)NC(=O)[C@H](CO)NC(=O)[C@@H](N)[C@@H](C)O)C(=O)N[C@@H](CCCc2cc(C)cc(C)c2)C(N)=O)cc1] | C₄₅H₆₃N₇O₁₀ | 861.5 | 862.9 [M + H]⁺ 431.9 [M + 2H]²⁺ |
| 15 | 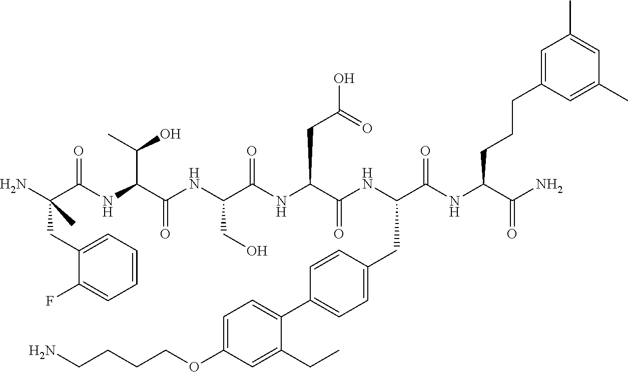 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@@](C)(N)Cc2ccccc2F)[C@@H](C)O)C(=O)N[C@@H](CCCc2cc(C)cc(C)c2)C(N)=O)cc1] | C₅₅H₇₃FN₈O₁₁ | 1040.5 | 521.6 [M + 2H]²⁺ |
| 16 | 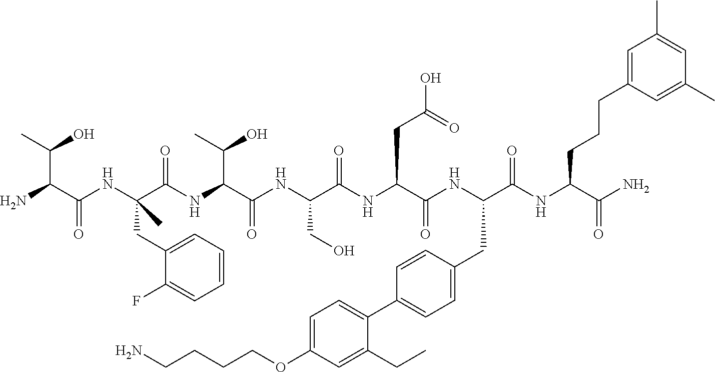 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](N)[C@@H](C)O)[C@@H](C)O)C(=O)N[C@@H](CCCc2cc(C)cc(C)c2)C(N)=O)cc1] | C₅₉H₈₀FN₉O₁₃ | 1141.6 | 572.0 [M + 2H]²⁺ |
| 17 | 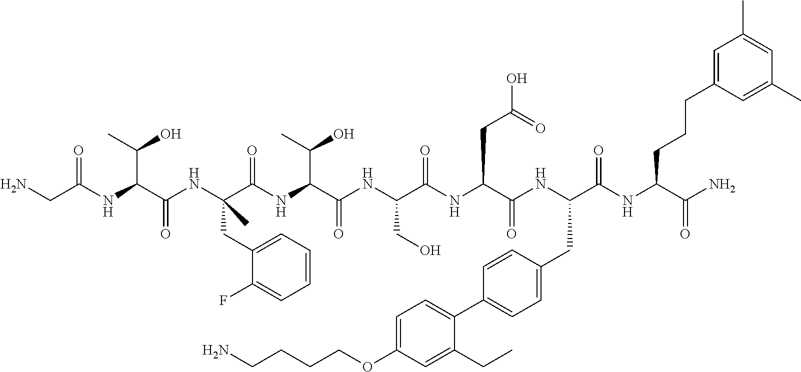 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CN)[C@@H](C)O)[C@@H](C)O)C(=O)N[C@@H](CCCc2cc(C)cc(C)c2)C(N)=O)cc1] | C₆₁H₈₃FN₁₀O₁₄ | 1198.6 | 600.6 [M + 2H]²⁺ |
| 18 | 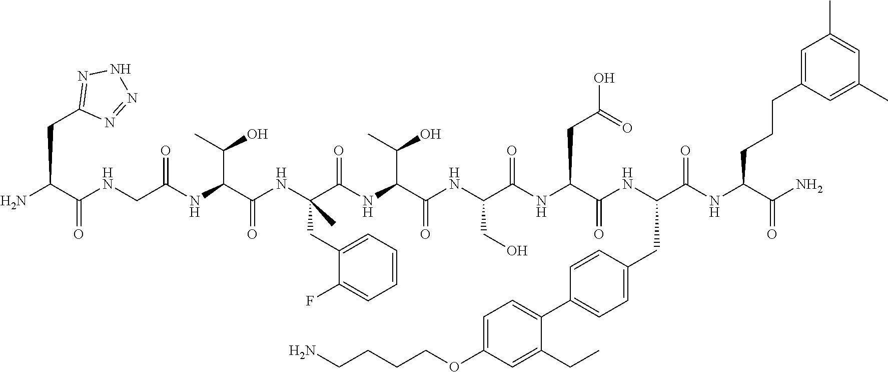 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(N)=O)cc1] | C₆₅H₈₈FN₁₅O₁₅ | 1337.7 | 670.1 [M + 2H]²⁺ |
| 19 | 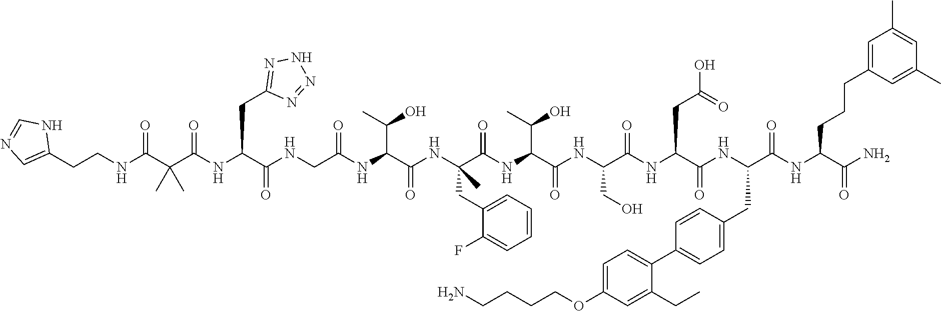 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(N)=O)cc1] | C₇₅H₁₀₁FN₁₈O₁₇ | 1544.8 | 773.7 [M + 2H]²⁺ |
| 20 | 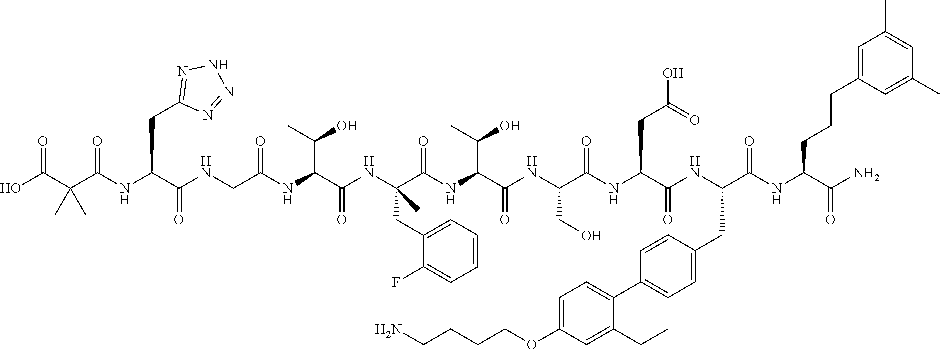 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)O)[C@@H](C)O)[C@@H](C)O)C(=O)N[C@@H](CCCc2cc(C)cc(C)c2)C(N)=O)cc1] | C₇₀H₉₄FN₁₅O₁₈ | 1451.7 | 726.9 [M + 2H]²⁺ |
| 21 | 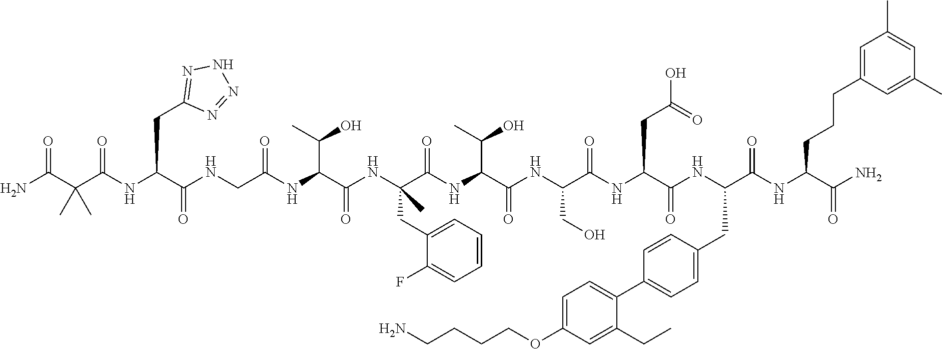 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(N)=O)cc1] | C₇₀H₉₅FN₁₆O₁₇ | 1450.7 | 1452.1 [M + H]²⁺ |
| 22 | 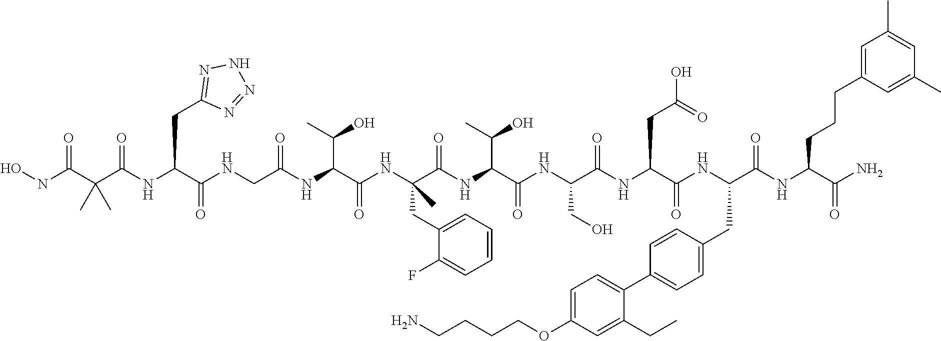 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NO)[C@@H](C)O)[C@@H](C)O)C(=O)N[C@@H](CCCc2cc(C)cc(C)c2)C(N)=O)cc1] | C₇₀H₉₄FN₁₆O₁₈ | 1465.7 | 734.4 [M + 2H]²⁺ |
| 23 | 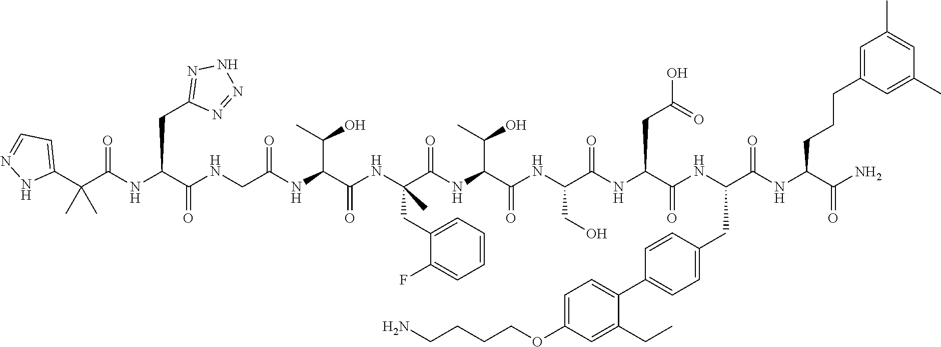 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)c2ccn[nH]2)[C@@H](C)O)[C@@H](C)O)C(=O)N[C@@H](CCCc2cc(C)cc(C)c2)C(N)=O)cc1] | C₇₂H₉₆FN₁₇O₁₆ | 1473.7 | 738.2 [M + 2H]²⁺ |
| 24 | 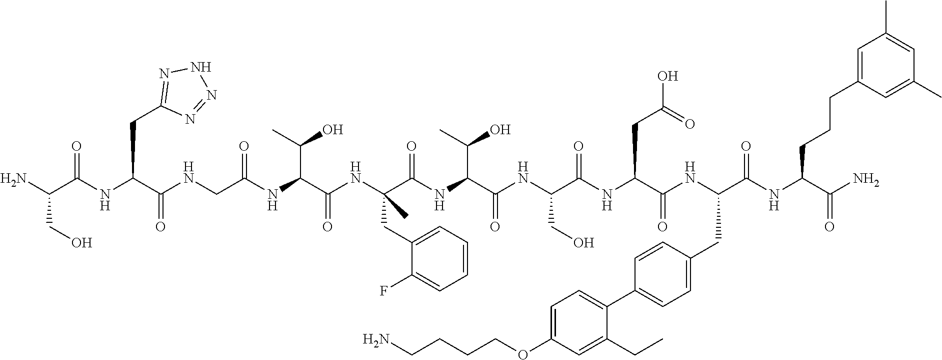 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)[C@@H](N)CO)[C@@H](C)O)[C@@H](C)O)C(=O)N[C@@H](CCCc2cc(C)cc(C)c2)C(N)=O)cc1] | C₆₈H₉₃FN₁₆O₁₇ | 1424.7 | 713.8 [M + 2H]²⁺ |
| 25 | 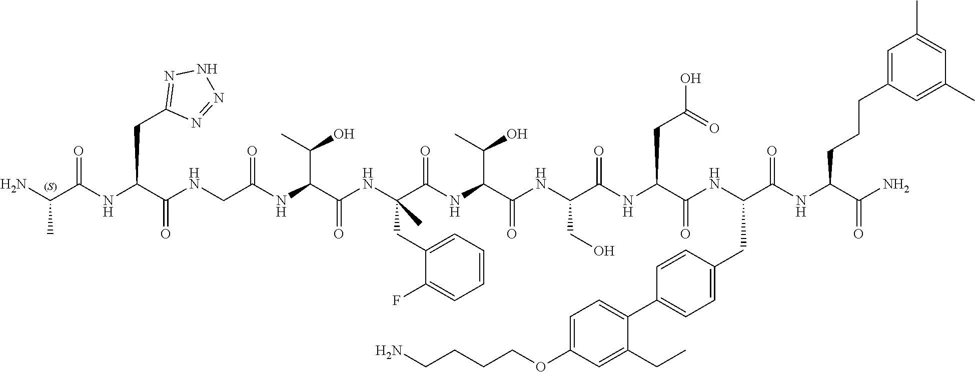 | C₆₈H₉₃FN₁₆O₁₆ | 1408.7 | 705.8 [M + 2H]²⁺ |
| 26 | 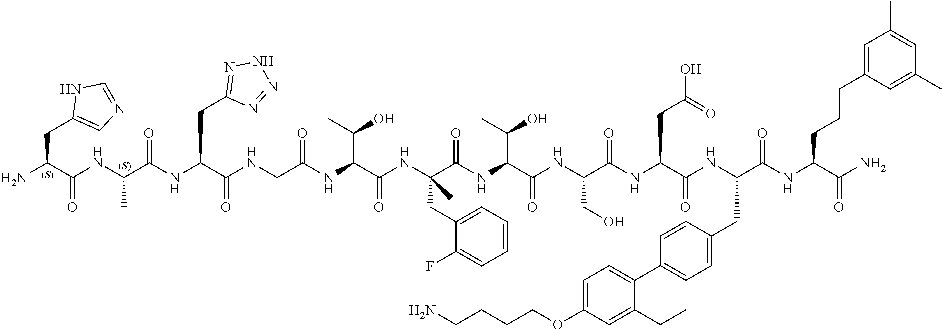 | C₇₄H₁₀₀FN₁₉O₁₇ | 1545.8 | 773.9 [M + 2H]²⁺ |
| 27 | 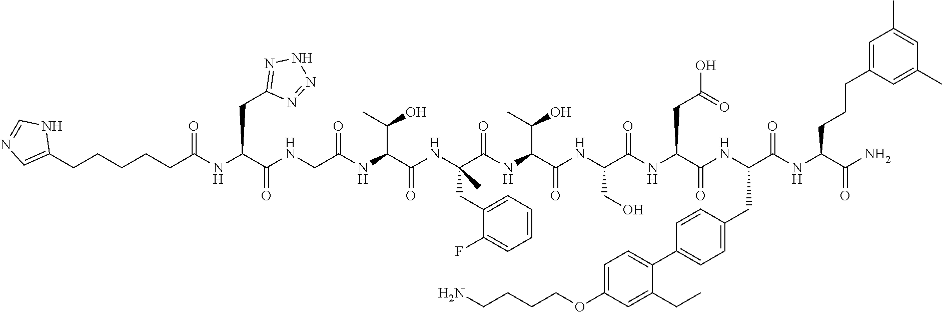 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(N)=O)cc1] | C₇₄H₁₀₀FN₁₇O₁₆ | 1501.8 | 752.0 [M + 2H]²⁺ |
| 28 | 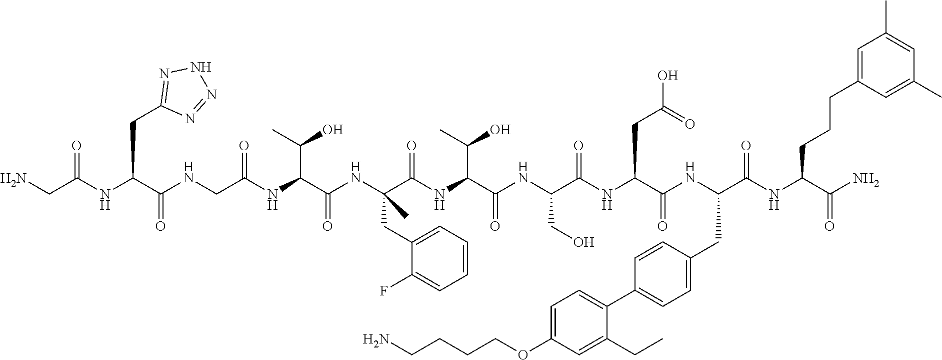 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CN)[C@@H](C)O)[C@@H](C)O)C(=O)N[C@@H](CCCc2cc(C)cc(C)c2)C(N)=O)cc1] | C₆₇H₉₁FN₁₆O₁₆ | 1394.7 | 698.6 [M + 2H]²⁺ |
| 29 | 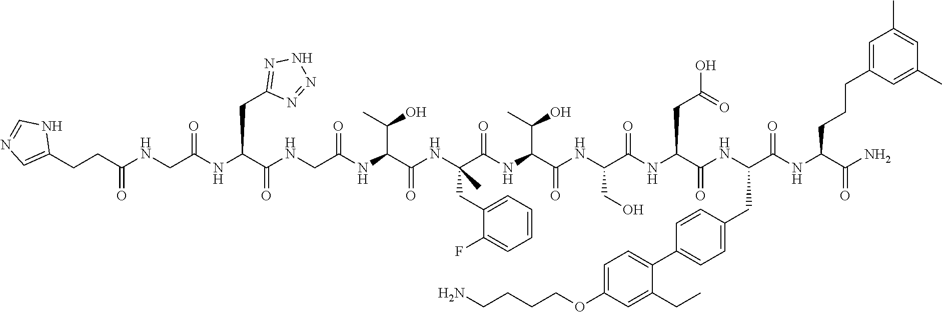 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CNC(=O)CCc2cnc[nH]2)[C@@H](C)O)[C@@H](C)O)C(=O)N[C@@H](CCCc2cc(C)cc(C)c2)C(N)=O)cc1] | C₇₃H₉₇FN₁₈O₁₇ | 1516.7 | 759.7 [M + 2H]²⁺ |
| 30 | 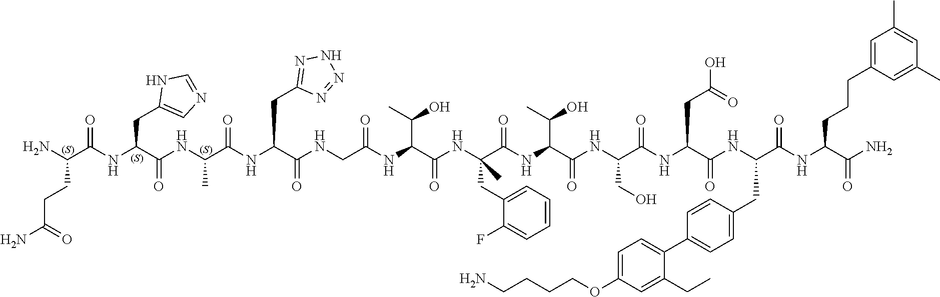 | C₇₉H₁₀₈FN₂₁O₁₉ | 1673.8 | 838.2 [M + 2H]²⁺ |
| 31 | 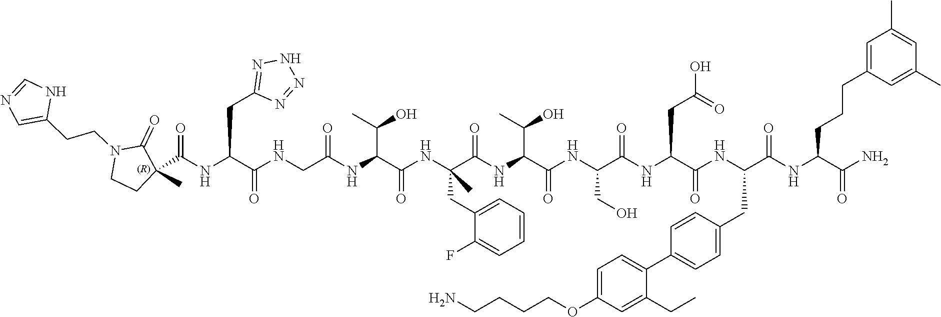 | C₇₆H₁₀₁FN₁₈O₁₇ | 1556.8 | 779.7 [M + 2H]²⁺ |
| 32 | 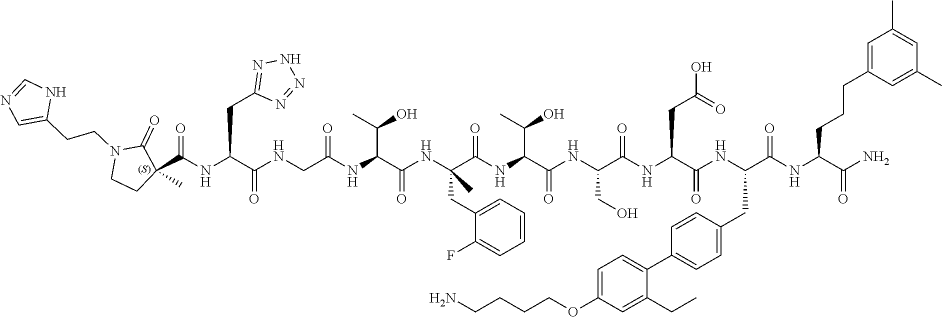 | C₇₆H₁₀₁FN₁₈O₁₇ | 1556.8 | 779.8 [M + 2H]²⁺ |
| 33 | 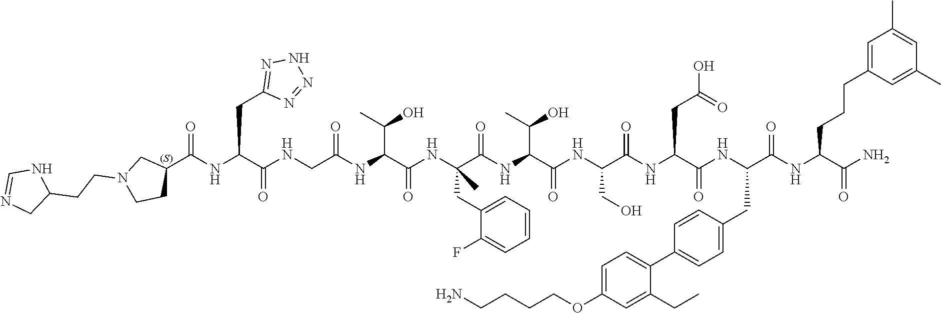 | C₇₅H₁₀₁FN₁₈O₁₆ | 1528.8 | 765.8 [M + 2H]²⁺ |
| 34 | 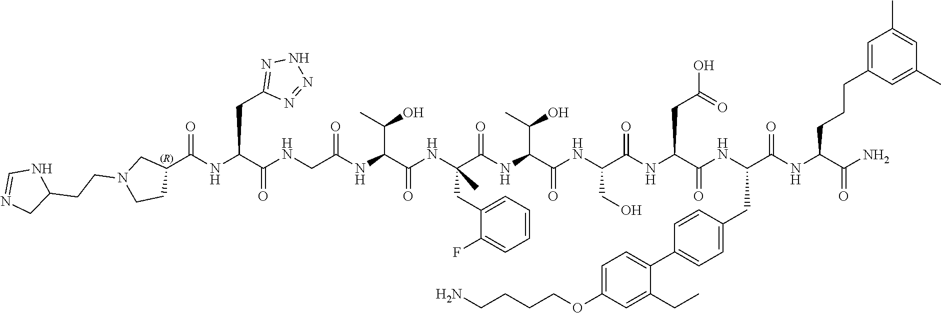 | C₇₅H₁₀₁FN₁₈O₁₆ | 1528.8 | 765.8 [M + 2H]²⁺ |
| 35 | 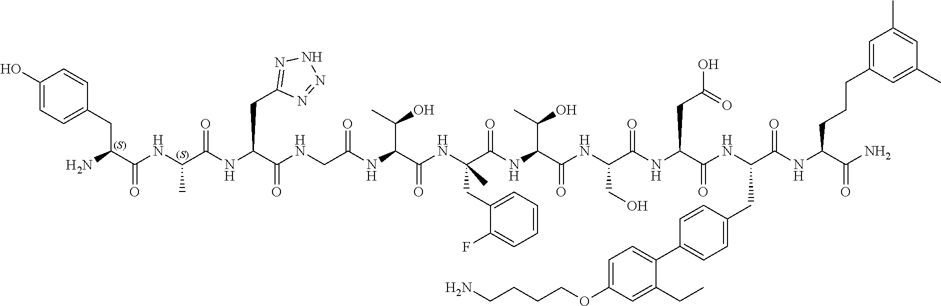 | C₇₇H₁₀₂FN₁₇O₁₈ | 1571.8 | 787.4 [M + 2H]²⁺ |
| 36 | 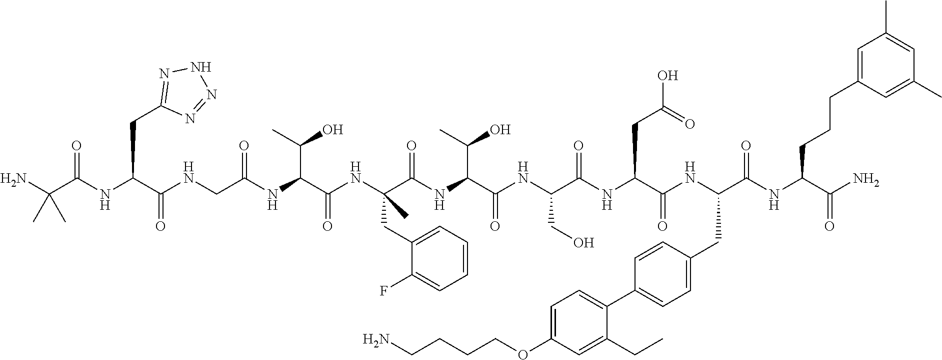 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)N)[C@@H](C)O)[C@@H](C)O)C(=O)N[C@@H](CCCc2cc(C)cc(C)c2)C(N)=O)cc1] | C₆₉H₉₅FN₁₆O₁₆ | 1422.7 | 712.7 [M + 2H]²⁺ |
| 36A | 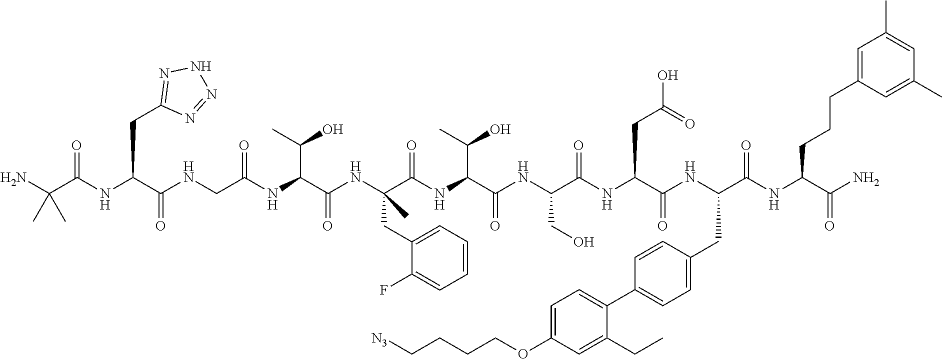 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)N)[C@@H](C)O)[C@@H](C)O)C(=O)N[C@@H](CCCc2cc(C)cc(C)c2)C(N)=O)cc1] | C₆₉H₉₃FN₁₈O₁₆ | 1448.7 | 1450.4 [M + H]⁺ |
| 37 | 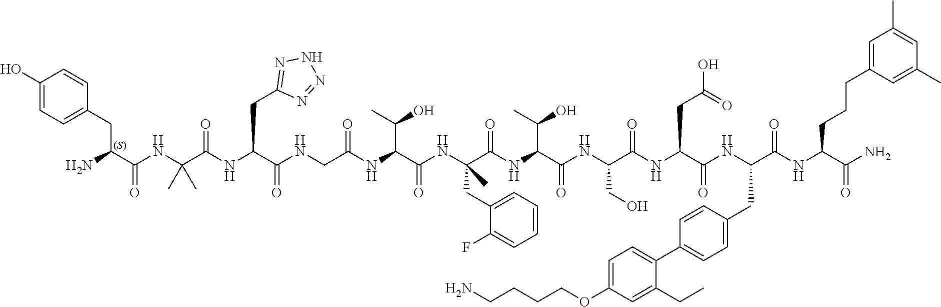 | C₇₈H₁₀₄FN₁₇O₁₈ | 1585.8 | 794.3 [M + 2H]²⁺ |
| 38 | 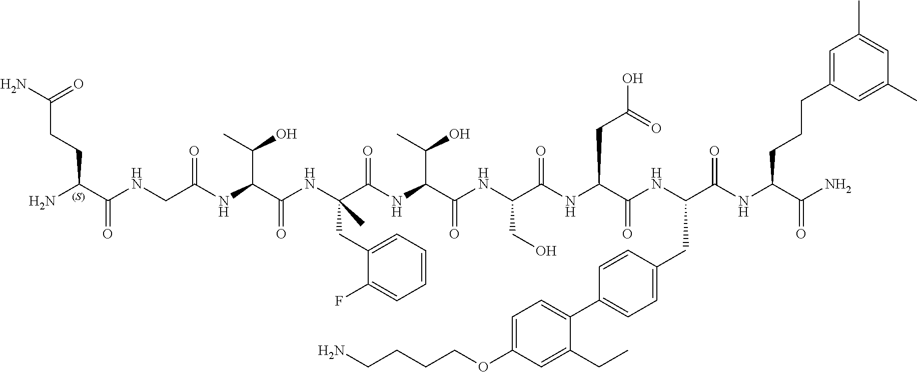 | C₆₆H₉₁FN₁₂O₁₆ | 1326.7 | 664.6 [M + 2H]²⁺ |
| 39 | 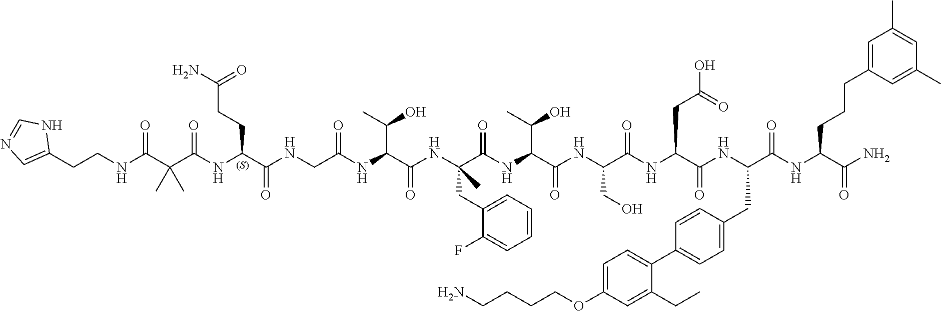 | C₇₆H₁₀₄FN₁₅O | 1533.8 | 768.3 [M + 2H]²⁺ |
| 40 | 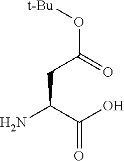 [SMILES: CC(C)(C)OC(=O)C[C@H](N)C(=O)O] | C₈H₁₅NO₄ | 189.2 | / |
| 41 | 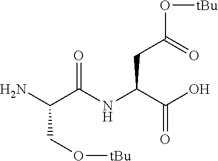 [SMILES: CC(C)(C)OC[C@H](N)C(=O)N[C@@H](CC(=O)OC(C)(C)C)C(=O)O] | C₁₅H₂₈N₂O₆ | 332.2 | 333.0 [M + H]⁺ |
| 42 | 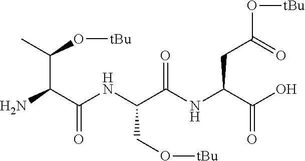 [SMILES: C[C@@H](OC(C)(C)C)[C@H](N)C(=O)N[C@@H](COC(C)(C)C)C(=O)N[C@@H](CC(=O)OC(C)(C)C)C(=O)O] | C₂₃H₄₃N₃O₈ | 489.3 | 490.2 [M + H]⁺ |
| 43 | 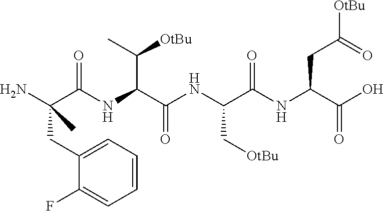 [SMILES: C[C@@H](OC(C)(C)C)[C@H](NC(=O)[C@@](C)(N)Cc1ccccc1F)C(=O)N[C@@H](COC(C)(C)C)C(=O)N[C@@H](CC(=O)OC(C)(C)C)C(=O)O] | C₃₃H₅₃FN₄O₉ | 668.4 | 669.3 [M + H]⁺ |
| 44 | 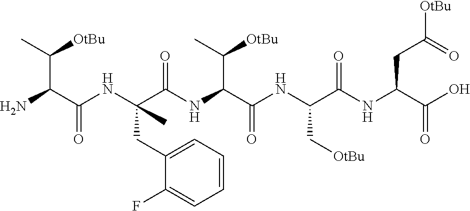 [SMILES: C[C@@H](OC(C)(C)C)[C@H](N)C(=O)N[C@@](C)(Cc1ccccc1F)C(=O)N[C@H](C(=O)N[C@@H](COC(C)(C)C)C(=O)N[C@@H](CC(=O)OC(C)(C)C)C(=O)O)[C@@H](C)OC(C)(C)C] | C₄₁H₆₈FN₅O₁₁ | 825.5 | 826.5 [M + H]⁺ |
| 45 | 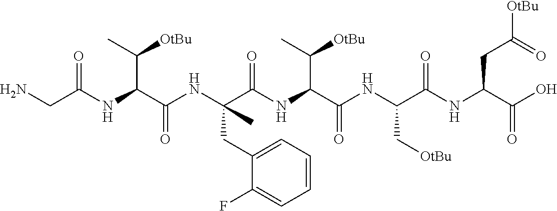 [SMILES: C[C@@H](OC(C)(C)C)[C@H](NC(=O)CN)C(=O)N[C@@](C)(Cc1ccccc1F)C(=O)N[C@H](C(=O)N[C@@H](COC(C)(C)C)C(=O)N[C@@H](CC(=O)OC(C)(C)C)C(=O)O)[C@@H](C)OC(C)(C)C] | C₄₃H₇₁FN₆O₁₂ | 882.5 | 883.4 [M + H]⁺ |
| 46 | 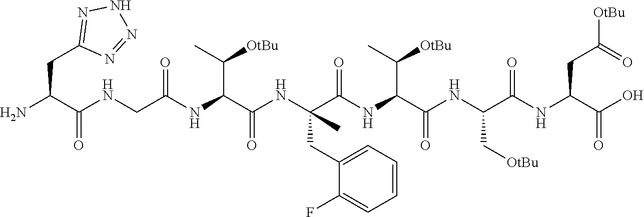 [SMILES: C[C@@H](OC(C)(C)C)[C@H](NC(=O)CNC(=O)[C@@H](N)Cc1nn[nH]n1)C(=O)N[C@@](C)(Cc1ccccc1F)C(=O)N[C@H](C(=O)N[C@@H](COC(C)(C)C)C(=O)N[C@@H](CC(=O)OC(C)(C)C)C(=O)O)[C@@H](C)OC(C)(C)C] | C₄₇H₇₆FN₁₁O₁₃ | 1021.6 | 1022.5 [M + H]⁺ |
| 47 | 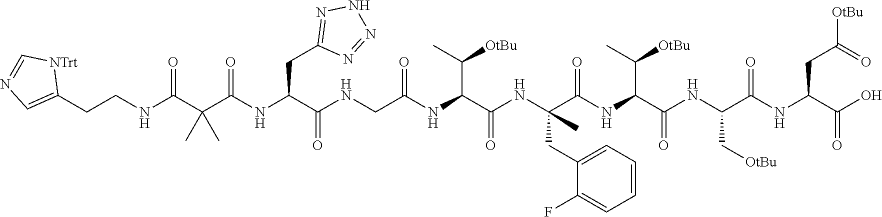 | C₇₆H₁₀₃FN₁₄O₁₅ | 1470.8 | 1471.8 [M + H]⁺ |
| 48 | 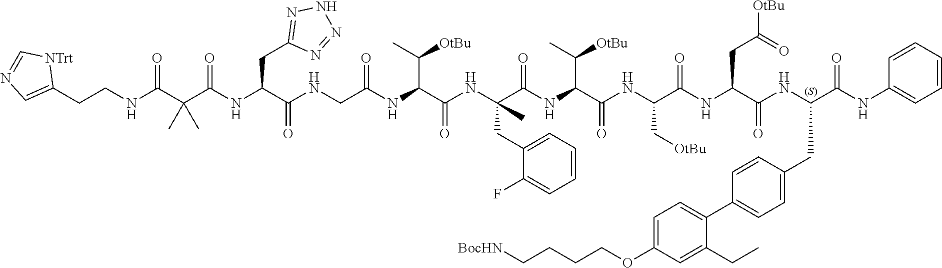 | C₁₀₈H₁₄₂FN₁₇O₁₈ | 1984.1 | 993.81 [M + 2H]²⁺ |
| 49 | 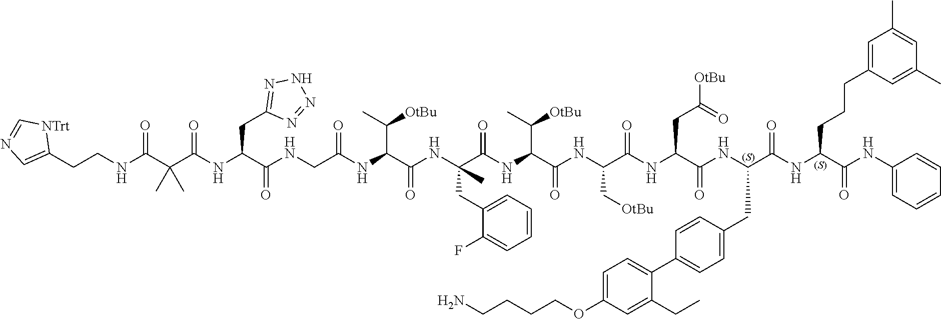 | C₁₁₆H₁₅₁FN₁₈O₁₇ | 2087.1 | 1044.8 [M + 2H]²⁺ |
| 50 | 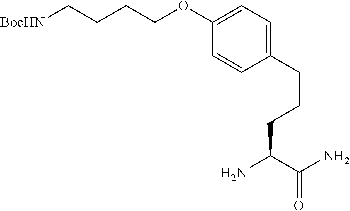 [SMILES: CC(C)(C)OC(=O)NCCCCOc1ccc(CCC[C@H](N)C(N)=O)cc1] | C₂₀H₃₃N₃O₄ | 379.5 | / |
| 51 | 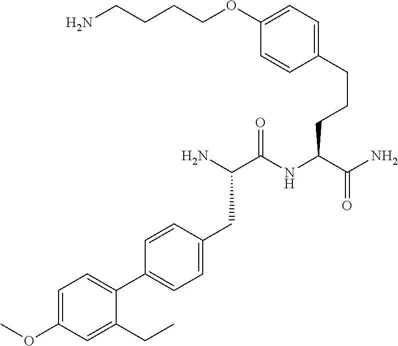 [SMILES: CCc1cc(OC)ccc1-c1ccc(C[C@H](N)C(=O)N[C@@H](CCCc2ccc(OCCCCN)cc2)C(N)=O)cc1] | C₃₃H₄₄N₄O₄ | 560.3 | 583.3 [M + Na]⁺ |
| 52 | 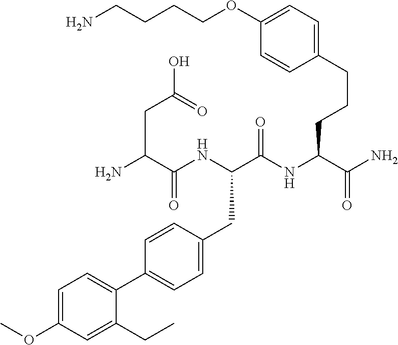 [SMILES: CCc1cc(OC)ccc1-c1ccc(C[C@H](NC(=O)C(N)CC(=O)O)C(=O)N[C@@H](CCCc2ccc(OCCCCN)cc2)C(N)=O)cc1] | C₃₇H₄₉N₅O₇ | 675.4 | 676.1 [M + H]⁺ |
| 53 | 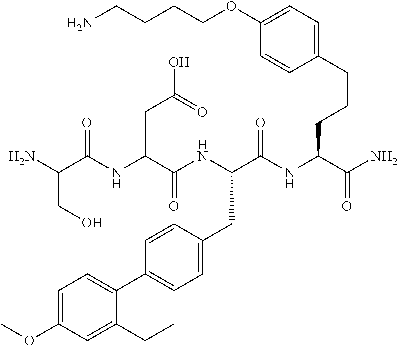 [SMILES: CCc1cc(OC)ccc1-c1ccc(C[C@H](NC(=O)C(CC(=O)O)NC(=O)C(N)CO)C(=O)N[C@@H](CCCc2ccc(OCCCCN)cc2)C(N)=O)cc1] | C₄₀H₅₄N₆O₉ | 762.4 | 763.4 [M + H]⁺ |
| 54 | 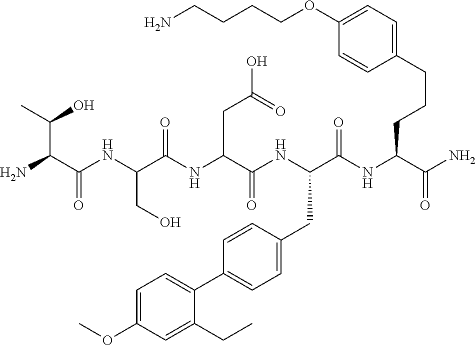 [SMILES: CCc1cc(OC)ccc1-c1ccc(C[C@H](NC(=O)C(CC(=O)O)NC(=O)C(CO)NC(=O)[C@@H](N)[C@@H](C)O)C(=O)N[C@@H](CCCc2ccc(OCCCCN)cc2)C(N)=O)cc1] | C₄₄H₆₁N₇O₁₁ | 863.4 | 864.4 [M + H]⁺ |
| 55 | 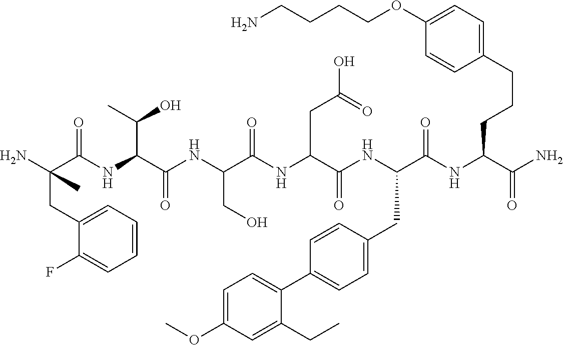 [SMILES: CCc1cc(OC)ccc1-c1ccc(C[C@H](NC(=O)C(CC(=O)O)NC(=O)C(CO)NC(=O)[C@@H](NC(=O)[C@@](C)(N)Cc2ccccc2F)[C@@H](C)O)C(=O)N[C@@H](CCCc2ccc(OCCCCN)cc2)C(N)=O)cc1] | C₅₄H₇₁FN₈O₁₂ | 1042.5 | 522.5 [M + 2H]²⁺ |
| 56 | 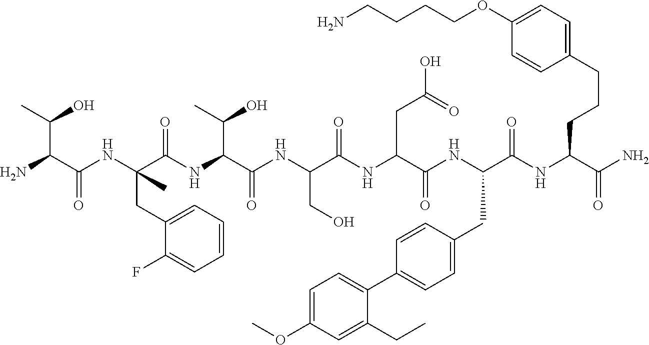 [SMILES: CCc1cc(OC)ccc1-c1ccc(C[C@H](NC(=O)C(CC(=O)O)NC(=O)C(CO)NC(=O)[C@@H](NC(=O)[C@](C)(Cc2ccccc2F)NC(=O)[C@@H](N)[C@@H](C)O)[C@@H](C)O)C(=O)N[C@@H](CCCc2ccc(OCCCCN)cc2)C(N)=O)cc1] | C₅₈H₇₈FN₉O₁₄ | 1143.6 | 573.0 [M + 2H]²⁺ |
| 57 | 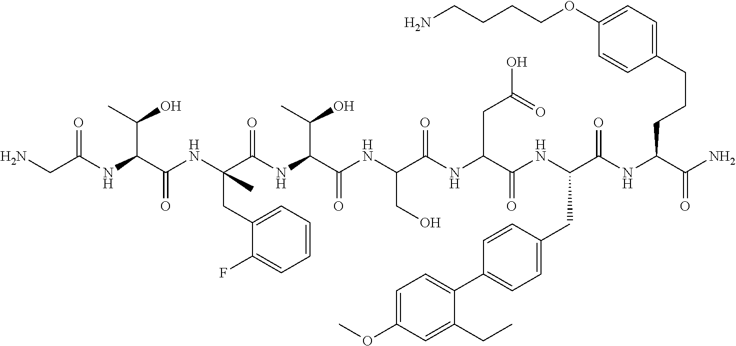 [SMILES: CCc1cc(OC)ccc1-c1ccc(C[C@H](NC(=O)C(CC(=O)O)NC(=O)C(CO)NC(=O)[C@@H](NC(=O)[C@](C)(Cc2ccccc2F)NC(=O)[C@@H](NC(=O)CN)[C@@H](C)O)[C@@H](C)O)C(=O)N[C@@H](CCCc2ccc(OCCCCN)cc2)C(N)=O)cc1] | C₆₀H₈₁FN₁₀O₁₅ | 1200.6 | 601.5 [M + 2H]²⁺ |
| 58 | 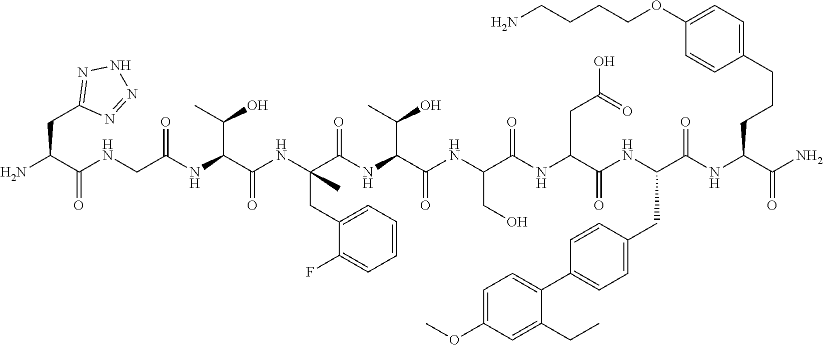 [SMILES: CCc1cc(OC)ccc1-c1ccc(C[C@H](NC(=O)C(CC(=O)O)NC(=O)C(CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2ccc(OCCCCN)cc2)C(N)=O)cc1] | C₆₄H₈₆FN₁₅O₁₆ | 1339.6 | 671.0 [M + 2H]²⁺ |
| 59 | 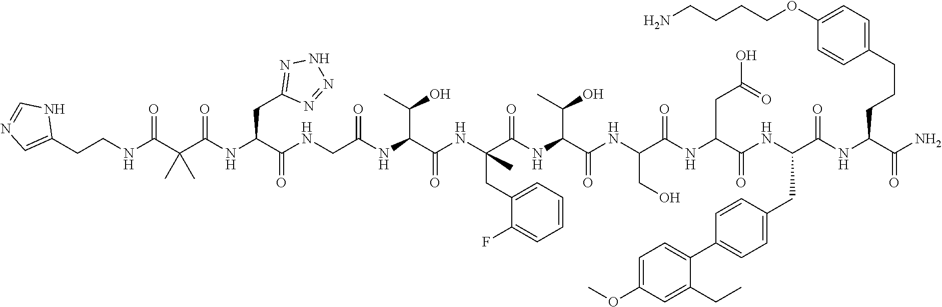 [SMILES: CCc1cc(OC)ccc1-c1ccc(C[C@H](NC(=O)C(CC(=O)O)NC(=O)C(CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCN)cc2)C(N)=O)cc1] | C₇₄H₉₉FN₁₈O₁₈ | 1546.7 | 774.6 [M + 2H]²⁺ |

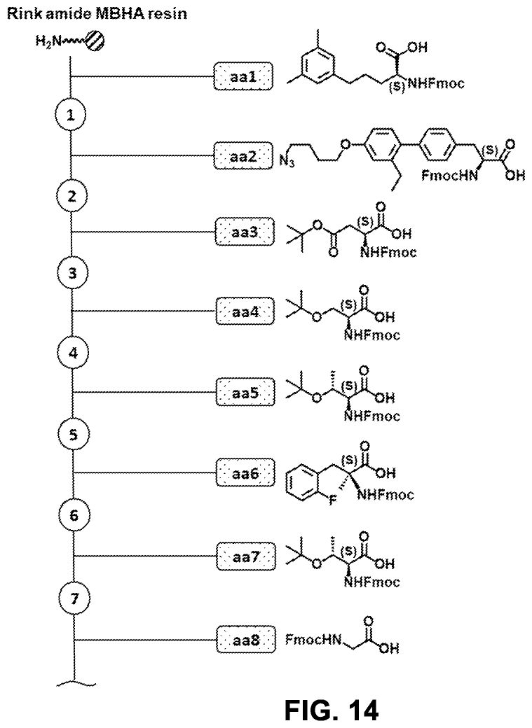

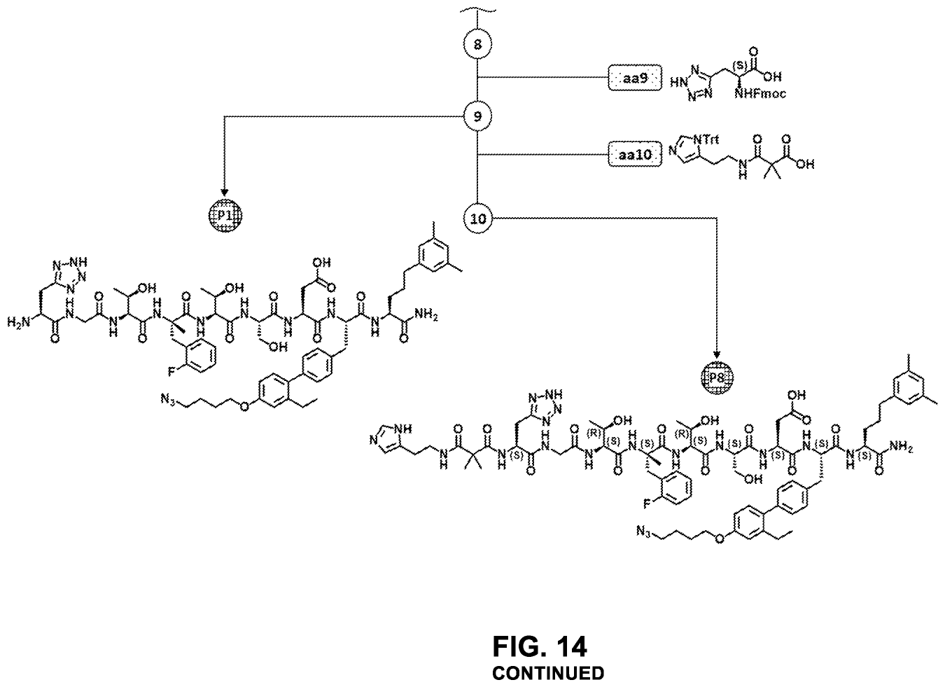

FIG. 14 depicts the sequence of steps for solid support synthesis of GLP1 peptidomimetic payloads P1 and P8 according to the disclosure.

### 3.1 Preparation of (3S,6S,9R,12S,15S,21S)-21-amino-3-(((S)-1-(((S)-1-amino-5-(3,5-dimethylphenyl)-1-oxopentan-2-yl)amino)-3-(4′-(4-aminobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-12-methyl-5,8,11,14,17,20-hexaoxo-22-(2H-tetrazol-5-yl)-4,7,10,13,16,19-hexaazadocosan-1-oic acid

The corresponding Fmoc-protected aa9-aa8-aa7-aa6-aa5-aa4-aa3-aa2-aa1 peptidyl Rink Amide MBHA Resin (9, 16.59 μmol) was prepared as described in the general procedure of SPPS. The resin-bound peptide 9 was cleaved following the general procedure to give the crude product as a white solid. This crude product was dissolved in DMF (1 mL), and piperidine (14.13 mg, 165.94 μmol, 16.39 μL, 10.0 eq.) was added in one portion at 20° C. under nitrogen. The mixture was stirred at 20° C. for 2 hours. The mixture was concentrated in vacuum to give the residue. The residue was purified by prep-HPLC (column: mobile phase: [water (0.1% TFA)-ACN]; B %: 30%-60%, 60 min) to afford pure product. The product was suspended in water (10 mL), the mixture frozen in a dry-ice/ethanol bath, and then lyophilized to dryness to afford the desired product P1 (1.02 mg, 7.48e-1 μmol, 4.50% yield, 100% purity) as a white solid. HRMS (ESI): mass calcd. for C₆₅H₈₇FN₁₇O₁₅ 1364.6552, m/z found 1364.4887 [M+H]⁺.

HPLC: RT=10.15 min, Reverse phase HPLC was carried out using a MERCK, RP-18e 25-2 mm column, with a flow rate of 1.2 mL/min, eluting with a gradient of 10% to 80% acetonitrile containing 0.02% TFA (solvent B) and water containing 0.04% TFA (solvent A).

### 3.2 Preparation of (8S,14S,17S,20S,23S,26S)-8-((2H-tetrazol-5-yl)methyl)-26-(((S)-1-(((S)-1-amino-5-(3,5-dimethylphenyl)-1-oxopentan-2-yl)amino)-3-(4′-(4-aminobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P8)

The corresponding aa10-aa9-aa8-aa7-aa6-aa5-aa4-aa3-aa2-aa1 peptidyl Rink Amide MBHA Resin (10, 100.99 μmol) was assembled as described in the general procedure of SPPS. The resin-bound peptide 10 was cleaved following the general procedure to give the crude product as a white solid. The crude was purified by preparative HPLC using column: Luna 200*25 mm, C18, 10 μm; mobile phase: [water (0.1% TFA)-ACN]; B %: 30%-60%, 60 min to afford pure product. The product was suspended in water (10 mL), the mixture frozen in a dry-ice/acetone bath, and then lyophilized to dryness to afford the desired product P8 (25.00 mg, 17.49 μmol, 17.32% yield, 100% purity) as a white solid.

HRMS (ESI): mass calcd for C₇₅H₁₀₀FN₂₀O₁₇ 1571.7559, m/z found 1571.7589 [M+H]⁺.

HPLC: RT=10.14 min. Reverse phase HPLC was carried out using a YMC-Pack ODS-A 150*4.6 mm, 5 μm column, with a flow rate of 1.5 mL/min, eluting with a gradient of 10% to 80% acetonitrile containing 0.062% TFA (solvent B) and water containing 0.068% TFA (solvent A).

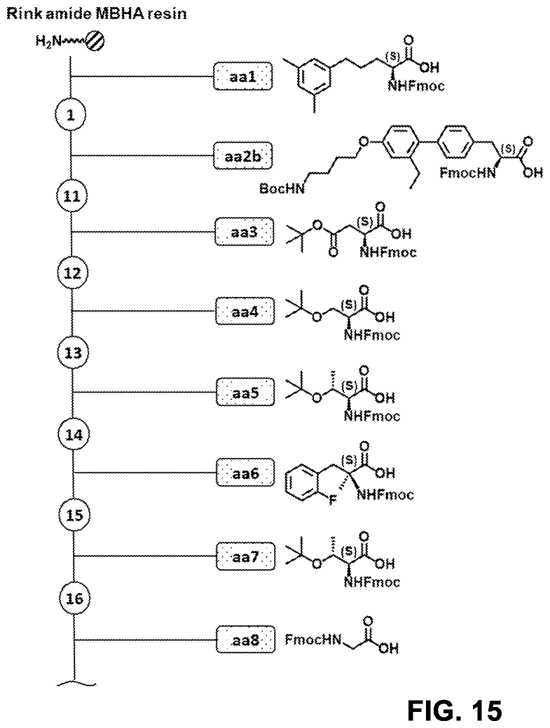

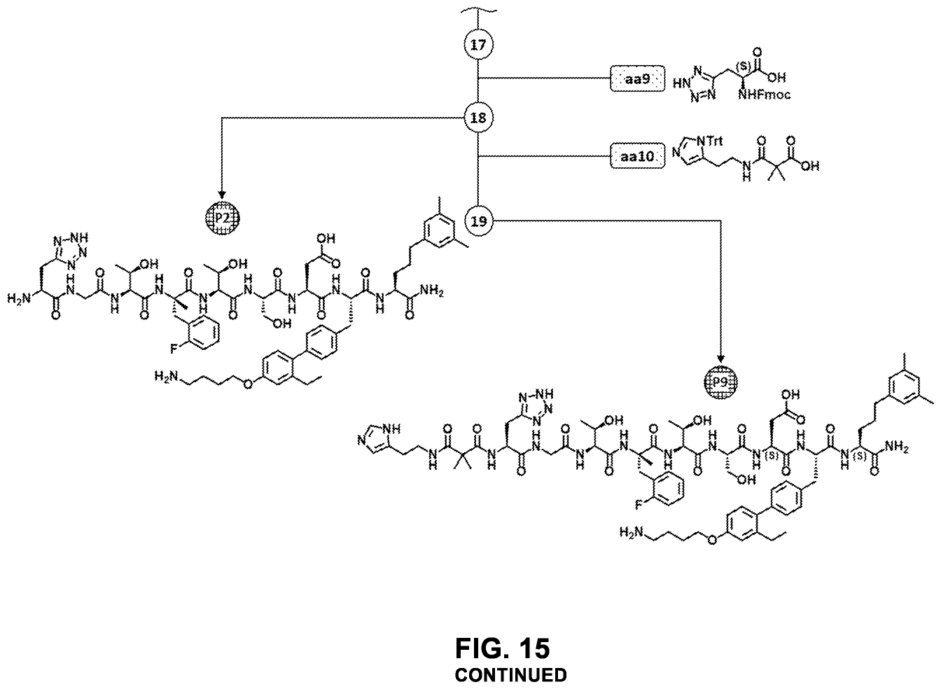

FIG. 15 depicts the sequence of steps for solid support synthesis of GLP1 peptidomimetic payloads P2 and P9 according to the disclosure.

### 3.3 Preparation of (8S,14S,17S,20R,23S,26S)-8-((2H-tetrazol-5-yl)methyl)-26-(((S)-1-(((S)-1-amino-5-(3,5-dimethylphenyl)-1-oxopentan-2-yl)amino)-3-(4′-(4-aminobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P2)

The corresponding aa9-aa8-aa7-aa6-aa5-aa4-aa3-aa2b-aa1 peptidyl Rink Amide MBHA Resin (18, 74.75 μmol) was prepared as described in the general procedure of SPPS. The resin-bound peptide 18 was cleaved following the general procedure to give the crude product as a white solid. The crude product was sent to prep-HPLC (column: mobile phase: [water (0.1% TFA)-ACN]; B %: 20%-50%, 60 min) to afford pure product. The product was suspended in water (100 mL), the mixture frozen in a dry-ice/acetone bath, and then lyophilized to dryness to afford the desired product P2 (11.56 mg, 8.45 μmol, 11.27% yield, 97.84% purity) was obtained as a white solid.

LCMS (ESI): RT=0.830 min, mass calcd. for C65H88FN15O15 1337.66 [M−4tBu-Boc+6H]⁺669.83 [M−4tBu-Boc+7H]2+, found 670.0 [M−4tBu-Boc+7H]2+. Reverse phase LC-MS was carried out using a Chromolith Flash RP-18e 25-3 mm column, with a flow rate of 1.5 mL/min, eluting with a gradient of 5% to 95% acetonitrile containing 0.04% TFA (solvent B) and water containing 0.06% TFA (solvent A).

HPLC: RT=7.77 min. Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.4 Preparation of (8S,14S,17S,20S,23S,26S)-8-((2H-tetrazol-5-yl)methyl)-26-(((S)-1-(((S)-1-amino-5-(3,5-dimethylphenyl)-1-oxopentan-2-yl)amino)-3-(4′-(4-aminobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P9)

The corresponding aa10-aa9-aa8-aa7-aa6-aa5-aa4-aa3-aa2b-aa1 peptidyl Rink Amide MBHA Resin (19) was prepared as described in the general procedure of SPPS. The resin-bound peptide 19 was cleaved following the general procedure to give the crude product as a white solid. The crude product was sent to prep-HPLC (TFA: mobile phase: [water (0.075% TFA)-ACN]; B %: 15%-45%, 55 min) to afford pure product. The product was suspended in water (20 mL), the mixture frozen in a dry-ice/acetone bath, and then lyophilized to dryness to afford the desired product P9 (125 mg, 79.82 μmol, 7.70% yield, 98.7% purity) was obtained as a white solid.

LCMS (ESI): RT=0.843 min, mass calcd. for C75H102FN18017 1545.77 [M−4tBu-Boc+6H]⁺773.385 [M−4tBu-Boc+7H]2+, found 773.9 [M−4tBu-Boc+7H]2+. Reverse phase LC-MS was carried out using a Chromolith Flash RP-18e 25-3 mm column, with a flow rate of 1.5 mL/min, eluting with a gradient of 5% to 95% acetonitrile containing 0.04% TFA (solvent B) and water containing 0.06% TFA (solvent A).

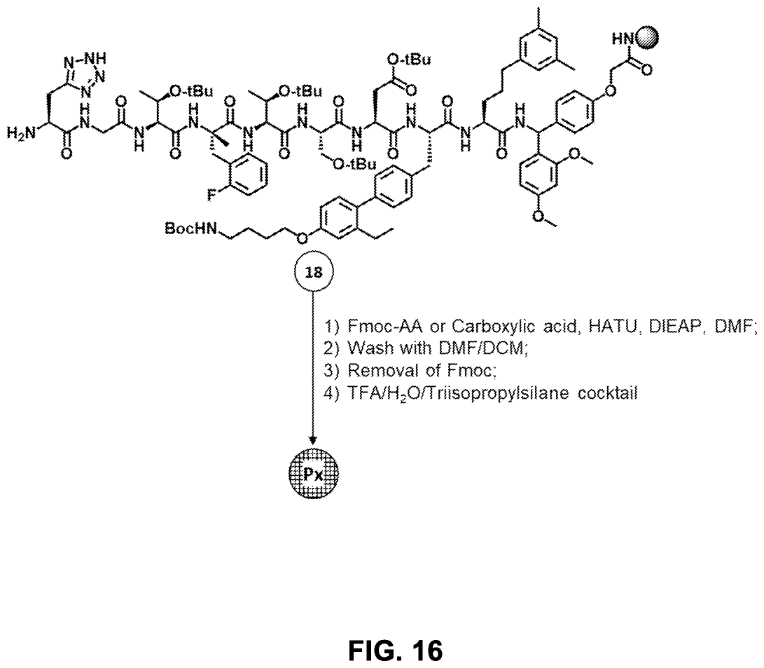

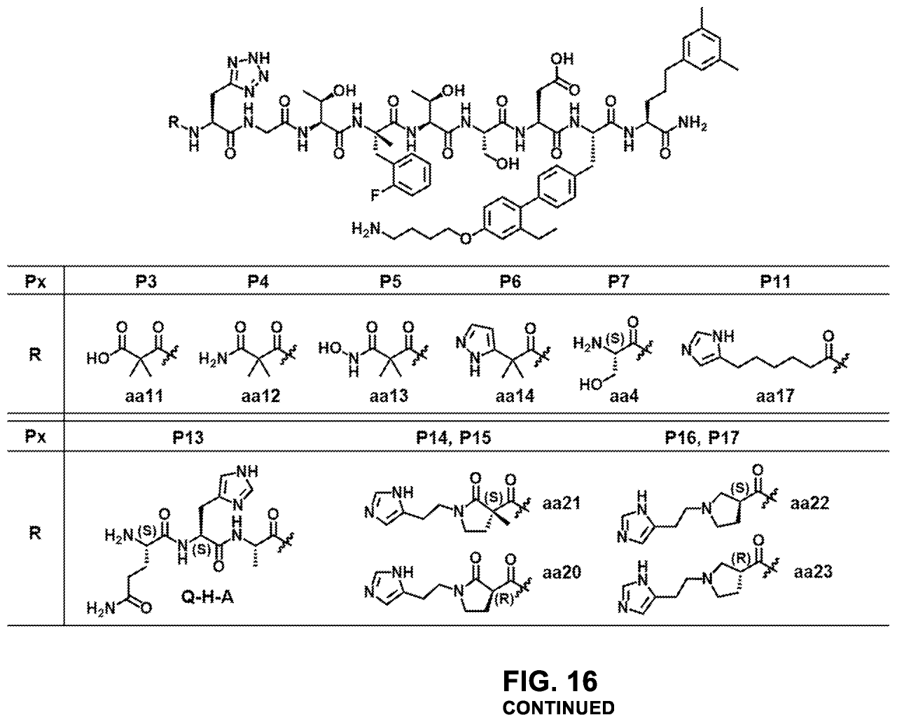

FIG. 16 depicts the sequence of steps for solid support synthesis of GLP1 peptidomimetic payloads P3, P4, P5, P6, P7, P11, P13, P14, P15, P16 and P17 according to the disclosure.

### 3.5 Preparation of 3-[[(1S)-2-[[2-[[(1S,2R)-1-[[(1S)-2-[[(1S,2R)-1-[[(1S)-2-[[(1S)-2-[[(1S)-1-[[4-[4-(4-aminobutoxy)-2-ethyl-phenyl]phenyl]methyl]-2-[[(1S)-1-carbamoyl-4-(3,5-dimethylphenyl) butyl]amino]-2-oxo-ethyl]amino]-1-(carboxymethyl)-2-oxo-ethyl]amino]-1-(hydroxymethyl)-2-oxo-ethyl]carbamoyl]-2-hydroxy-propyl]amino]-1-[(2-fluorophenyl)methyl]-1-methyl-2-oxo-ethyl]carbamoyl]-2-hydroxy-propyl]amino]-2-oxo-ethyl]amino]-2-oxo-1-(2H-tetrazol-5-ylmethyl)ethyl]amino]-2,2-dimethyl-3-oxo-propanoic acid (P3)

Starting from the corresponding aa9-aa8-aa7-aa6-aa5-aa4-aa3-aa2b-aa1 peptidyl Rink Amide MBHA Resin (18, 152.96 μmol) and aa11 (57.58 mg, 305.91 μmol, 2.0 eq.), the corresponding aa11-aa10-aa9-aa8-aa7-aa6-aa5-aa4-aa3-aa2b-aa1 peptidyl Rink Amide MBHA Resin (20, 119 μmol) was prepared as described in the general procedure of SPPS.

The corresponding resin-bound peptide 20 (119 μmol) was further cleaved following the general procedure to give the crude product as a white solid. The crude was purified by prep-HPLC (column: YMC-Exphere C18 10 μm 300*50 mm, 12 nm; mobile phase: [water(0.1% TFA)-ACN]; B %: 15%-45%, 55 min) to provide P3 (8 mg, 5.47 μmol, 3.58% yield, 99.37% purity) as a light yellow solid.

HPLC: RT=8.10 min. HPLC conditions: YMC-Pack ODS-A 150*4.6 mm, 5 μm column, flow rate of 1.5 mL/min, eluting with a gradient of 10% to 80% acetonitrile containing 0.12% TFA (solvent B) and water containing 0.1% TFA (solvent A).

LCMS: (ESI): RT=0.841 min, m/z calcd. for C18H21NO2 1452.69 [M+H]⁺, found 727.3 [M+2H]2+; LCMS conditions: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

### 3.6 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-2-[(3-amino-2,2-dimethyl-3-oxo-propanoyl)amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-oxo-butanoic acid (P4)

Starting from the corresponding aa9-aa8-aa7-aa6-aa5-aa4-aa3-aa2b-aa1 peptidyl Rink Amide MBHA Resin compound 18 (192.73 μmol) and aa12 (75.82 mg, 578.18 μmol, 3 eq.), the corresponding aa12-aa10-aa9-aa8-aa7-aa6-aa5-aa4-aa3-aa2b-aa1 peptidyl Rink Amide MBHA Resin (21, 192.73 μmol) was obtained as described in the general procedure of SPPS.

The corresponding resin-bound peptide 21 (192.73 μmol) was further cleaved following the general procedure to give the crude product as a white solid. The crude was purified by prep-HPLC (column: YMC-Exphere C18 10 μm 300*50 mm, 12 nm; mobile phase: [water(0.1% TFA)-ACN]; B %: 15%-45%, 55 min) to provide P4 (35 mg, 24.03 μmol, 8.73% yield, 99.66% purity) as a light yellow solid.

LCMS: (ESI): RT=0.861 min, mass calcd. for C76H103FN18O17 726.36 m/z [M+2H]2+; found 726.7 m/z [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.854 min, mass calcd. for C76H103FN18017 726.36 m/z [M+2H]2+; found 726.7 m/z [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=7.90 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.7 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-(hydroxyamino)-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (P5)

Starting from the corresponding aa9-aa8-aa7-aa6-aa5-aa4-aa3-aa2b-aa1 peptidyl Rink Amide MBHA Resin compound 18 (152.96 μmol) and aa13 (60 mg, 259.47 μmol, 1.7 eq.), the corresponding aa13-aa10-aa9-aa8-aa7-aa6-aa5-aa4-aa3-aa2b-aa1 peptidyl Rink Amide MBHA Resin (22, 152.67 μmol) was obtained as described in the general procedure of SPPS.

The corresponding resin-bound peptide 22 was cleaved following the general procedure to give the crude product as a white solid. The crude was purified by prep-HPLC (column: YMC-Exphere C18 10 μm 300*50 mm, 12 nm; mobile phase: [water(0.1% TFA)-ACN]; B %: 15%-45%, 55 min) to provide P5 (4 mg, 2.66 μmol, 42.12% yield, 97.7% purity) as a light yellow solid.

LCMS: (ESI): RT=0.705 min, mass calcd. for C₇₀H₉₆FN₁₆O₁₈ 733.85 m/z [M+2H]²⁺; found 718.2 m/z [M−2OH+4H]²⁺; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.847 min, mass calcd. for C₇₀H₉₆FN₁₆O₁₈ 733.85 m/z [M+2H]²⁺; found 735.2 m/z [M+2H]²⁺; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=8.00 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.8 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[2-methyl-2-(1H-pyrazol-5-yl) propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (P6)

Starting from the corresponding aa9-aa8-aa7-aa6-aa5-aa4-aa3-aa2b-aa1 peptidyl Rink Amide MBHA Resin compound 18 (82.60 μmol) and aa14 (25.47 mg, 165.19 μmol, 2 eq.), the corresponding aa14-aa10-aa9-aa8-aa7-aa6-aa5-aa4-aa3-aa2b-aa1 peptidyl Rink Amide MBHA Resin (23, 82.60 μmol) was obtained as described in the general procedure of SPPS.

The corresponding resin-bound peptide 23 (82.60 μmol) was cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P6 (9 mg, 6.10 μmol, 7.40% yield, 97.34% purity) as a light yellow solid.

LCMS: (ESI): RT=0.856 min, mass calcd. for C72H98FN17016 737.87 m/z [M-4tBu-Boc-C17H17NO4+8H]2+, rink amide (C17H17NO4, exact mass=299.12); found 738.2 m/z [M−4tBu-Boc-C17H17NO4+8H]2+, rink amide (C17H17NO4, exact mass=299.12); LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.856 min, mass calcd. for C72H98FN17016 737.87 m/z [M-4tBu-Boc-C17H17NO4+8H]2+, rink amide (C17H17NO4, exact mass=299.12); found 738.2 m/z [M−4tBu-Boc-C17H17NO4+8H]2+, rink amide (C17H17NO4, exact mass=299.12); LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=8.16 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.9 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-2-[[(2S)-2-amino-3-hydroxy-propanoyl]amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-oxo-butanoic acid (P7)

Starting from the corresponding aa9-aa8-aa7-aa6-aa5-aa4-aa3-aa2b-aa1 peptidyl Rink Amide MBHA Resin (18, 76.48 μmol) and aa4 (87.98 mg, 229.44 μmol, 3 eq.). The corresponding aa4-aa10-aa9-aa8-aa7-aa6-aa5-aa4-aa3-aa2b-aa1 peptidyl Rink Amide MBHA Resin (24, 76.48 μmol) was obtained as described in the general procedure of SPPS.

The corresponding resin-bound peptide 24 (76.48 μmol) was cleaved following the general procedure to give the crude product as a white solid. The crude was purified by prep-HPLC (column: Waters Xbridge Prep OBD C18 150*40 mm*10 μm; mobile phase: [water(0.1% TFA)-ACN]; B %: 28%-58%, 30 min) to provide P7 (17 mg, 11.16 μmol, 14.59% yield, 93.57% purity) as a white solid.

LCMS: (ESI): RT=2.675 min, m/z calcd. for C68H95FN16017, 713.35 [M+2H]2+, m/z found 713.8 [M+2H]2+; Mobile Phase: 1.5 ML/4 L TFA in water (solvent A) and 0.75 ML/4 L TFA in acetonitrile (solvent B),using the elution gradient 10%-80% (solvent B) over 6 minutes and holding at 80% for 0.5 minutes at a flow rate of 0.8 ml/min;

LCMS: (ESI): RT=0.757 min, m/z calcd. for C68H95FN16017, 713.35 [M+2H]2+, m/z found 713.8 [M+2H]2+; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A);

HPLC (Rt=7.58 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.10 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[6-(1H-imidazol-5-yl) hexanoylamino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P11)

Starting from the corresponding aa9-aa8-aa7-aa6-aa5-aa4-aa3-aa2b-aa1 peptidyl Rink Amide MBHA Resin (18, 74.75 μmol) and aa17 (70.13 mg, 165.19 μmol, 2 eq.), the corresponding aa17-aa10-aa9-aa8-aa7-aa6-aa5-aa4-aa3-aa2b-aa1 peptidyl Rink Amide MBHA Resin (27, 74.75 μmol) was obtained as described in the general procedure of SPPS.

The corresponding resin-bound peptide compound 27 (74.75 μmol) was cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P7 (9 mg, 5.80 μmol, 7.05% yield, 96.92% purity) as a white solid.

HPLC: RT=7.72 min. HPLC conditions: YMC-Pack ODS-A 150*4.6 mm, 5 μm column, flow rate of 1.5 mL/min, eluting with a gradient of 10% to 80% acetonitrile containing 0.12% TFA (solvent B) and water containing 0.1% TFA (solvent A).

LCMS: (ESI): RT=0.828 min, m/z calcd. for C18H21NO2 1502.75 [M+H]⁺, found 752.3 [M+2H]2+; LCMS conditions: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

### 3.11 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-2-[[(2S)-2-[[(2S)-2-[[(2S)-2,5-diamino-5-oxo-pentanoyl]amino]-3-(1H-imidazol-4-yl)propanoyl]amino]propanoyl]amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxybutanoyl]amino]-3-hydroxy-propanoyl]amino]-4-oxo-butanoic acid (P13)

Starting from the corresponding aa9-aa8-aa7-aa6-aa5-aa4-aa3-aa2b-aa1 peptidyl Rink Amide MBHA Resin compound 18 (82.60 μmol), aa15 (77.14 mg, 247.79 μmol, 3 eq.), aa16 (93.40 mg, 247.49 μmol, 3 eq.), and aa19 (40.63 mg, 165.00 μmol, 2 eq.), the corresponding aa19-aa16-aa15-aa9-aa8-aa7-aa6-aa5-aa4-aa3-aa2b-aa1 peptidyl Rink Amide MBHA Resin (30, 82.50 μmol) was prepared as described in the general procedure of SPPS.

The corresponding resin-bound peptide 30 (82.50 μmol) was cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P13 (10 mg, 5.69 μmol, 6.90% yield, 95.28% purity) as a white solid.

LCMS: (ESI): RT=0.807 min, mass calcd. for C79H110FN21019 837.9 m/z [M-4tBu-2Boc-C17H17NO4+9H]2+, rink amide (C17H17NO4, exact mass=299.12); found 838.4 m/z [M−4tBu-Boc-C17H17NO4+9H]2+, rink amide (C17H17NO4, exact mass=299.12); LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.820 min, mass calcd. for C79H110FN21019 837.9 m/z [M-4tBu-2Boc-C17H17NO4+9H]2+, rink amide (C17H17NO4, exact mass=299.12); found 838.3 m/z [M−4tBu-Boc-C17H17NO4+9H]2+, rink amide (C17H17NO4, exact mass=299.12); LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=7.37 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.12 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[(3R)-1-[2-(1H-imidazol-5-yl) ethyl]-3-methyl-2-oxo-pyrrolidine-3-carbonyl]amino]-3-(2H-tetrazol-5-yl) propanoyl] amino]acetyl]amino]butanoyl]amino]-2-methyl-propanoyl]amino]-3-hydroxy-butanoyl]amino]-3-hydroxypropanoyl]amino]-4-oxo-butanoic acid (P14)

Starting from the corresponding aa9-aa8-aa7-aa6-aa5-aa4-aa3-aa2b-aa1 peptidyl Rink Amide MBHA Resin (18, 82.60 μmol) and aa20 (79.22 mg, 165.19 μmol, 2 eq.), the corresponding aa20-aa9-aa8-aa7-aa6-aa5-aa4-aa3-aa2b-aa1 peptidyl Rink Amide MBHA Resin (31, 82.60 μmol) was prepared as described in the general procedure of SPPS.

The corresponding resin-bound peptide 31 (82.60 μmol) was further cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P14 (8 mg, 3.85 μmol, 6.2% yield, 93.37% purity) as a white solid.

LCMS: (ESI): RT=0.840 min, mass calcd. for C76H103FN18017 779.39 m/z [M+2H]2+; found 779.9 m/z [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.864 min, mass calcd. for C76H103FN18017 779.39 m/z [M+2H]2+; found 779.9 m/z [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=7.60 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.13 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[(3R)-1-[2-(1H-imidazol-5-yl) ethyl]-3-methyl-2-oxo-pyrrolidine-3-carbonyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (P15)

Starting from the corresponding aa9-aa8-aa7-aa6-aa5-aa4-aa3-aa2b-aa1 peptidyl Rink Amide MBHA Resin (18, 82.60 μmol) and aa21 (79.22 mg, 165.19 μmol, 2 eq.), the corresponding aa21-aa9-aa8-aa7-aa6-aa5-aa4-aa3-aa2b-aa1 peptidyl Rink Amide MBHA Resin (32, 82.60 μmol) was prepared as described in the general procedure of SPPS.

The corresponding resin-bound peptide 32 (82.60 μmol) was further cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P15 (10.5 mg, 6.54 μmol, 7.91% yield, 96.96% purity) as a white solid.

LCMS: (ESI): RT=0.828 min, mass calcd. for C76H103FN18017 779.39 m/z [M+2H]2+; found 779.8 m/z [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.830 min, mass calcd. for C76H103FN18017 779.39 m/z [M+2H]2+; found 779.8 m/z [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=7.62 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.14 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[(3S)-1-[2-(1H-imidazol-5-yl) ethyl]pyrrolidine-3-carbonyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P16)

Starting from the corresponding aa9-aa8-aa7-aa6-aa5-aa4-aa3-aa2b-aa1 peptidyl Rink Amide MBHA Resin (18 (141.13 μmol) and aa22 (127.46 mg, 282.27 μmol, 2.0 eq.), the corresponding aa22-aa9-aa8-aa7-aa6-aa5-aa4-aa3-aa2b-aa1 peptidyl Rink Amide MBHA Resin (33, 141.13 μmol) was prepared as described in the general procedure of SPPS.

The corresponding resin-bound peptide 33 (141.13 μmol) was further cleaved following the general procedure to give the crude product as a white solid. The crude was purified by prep-HPLC (column: YMC-Exphere C18 10 μm 300*50 mm, 12 nm; mobile phase: [water(0.1% TFA)-ACN]; B %: 15%-45%, 55 min) to provide P16 (15 mg, 9.71 μmol, 6.88% yield, and 99% purity) as a white solid.

LCMS (ESI): RT=2.361 min, m/z calcd. for C75H103FN18016, 1530.76 M-Boc-4tBu+2H]2+, m/z found 765.8, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min.ESI source, Positive ion mode; Wavelength 220 nm&254 nm, OvenTemperature 50° C.

LCMS (ESI): RT=0.755 min, m/z calcd. for C75H103FN18016, 1530.76 M-Boc-4tBu+2H]2+, m/z found 765.8, Mobile Phase: 1.5 ML/4LTFA in water (solvent A) and 0.75 ML/4 L TFA in acetonitrile (solvent B),using the elution gradient 5%-95% (solvent B) over 0.7 minutes and holding at 95% for 0.4 minutes at a flow rate of 1.5 mL/min; Column: Agilent Pursult 5 C18 20*2.0 mm Wavelength: UV 220 nm; Column temperature: 50° C.; MS ionization: ESI

HPLC: RT=7.18 min Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm Wavelength: UV 220 nm, 215 nm& 254 nm Column temperature: 40° C.

### 3.15 Preparation of (3S,9S,12S,15S,18S,21S)-tert-butyl1-((R)-1-(2-(1H-imidazol-5-yl)ethyl) pyrrolidin-3-yl)-3-((2H-tetrazol-5-yl)methyl)-21-(((S)-1-(((S)-1-amino-5-(3,5-dimethylphenyl)-1-oxopentan-2-yl)amino)-3-(4′-(4-aminobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-18-(hydroxymethyl)-12-methyl-1,4,7,10,13,16,19-heptaoxo-2,5,8,11,14,17,20-heptaazatricosan-23-oate (P17)

Starting from the corresponding aa9-aa8-aa7-aa6-aa5-aa4-aa3-aa2b-aa1 peptidyl Rink Amide MBHA Resin compound 18 (101.97 μmol) and aa23 (80 mg, 177.16 μmol, 1.74 eq.), the corresponding aa23-aa9-aa8-aa7-aa6-aa5-aa4-aa3-aa2b-aa1 peptidyl Rink Amide MBHA Resin (34, 101.88 μmol) was prepared as described in the general procedure of SPPS.

The corresponding resin-bound peptide 34 (101.88 μmol) was further cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P17 (18 mg, 11.65 μmol, 11.43% yield, 99% purity) as a white solid.

LCMS (ESI): RT=0.828 min, m/z calcd. for C75H103FN18016, 1530.76 M-Boc-4tBu+2H]2+, m/z found 765.8, Mobile Phase: 1.5 ML/4LTFA in water (solvent A) and 0.75 ML/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm Wavelength: UV 220 nm; Column temperature: 50° C.; MS ionization: ESI

HPLC: RT=7.36 min Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm Wavelength: UV 220 nm, 215 nm& 254 nm Column temperature: 40° C.

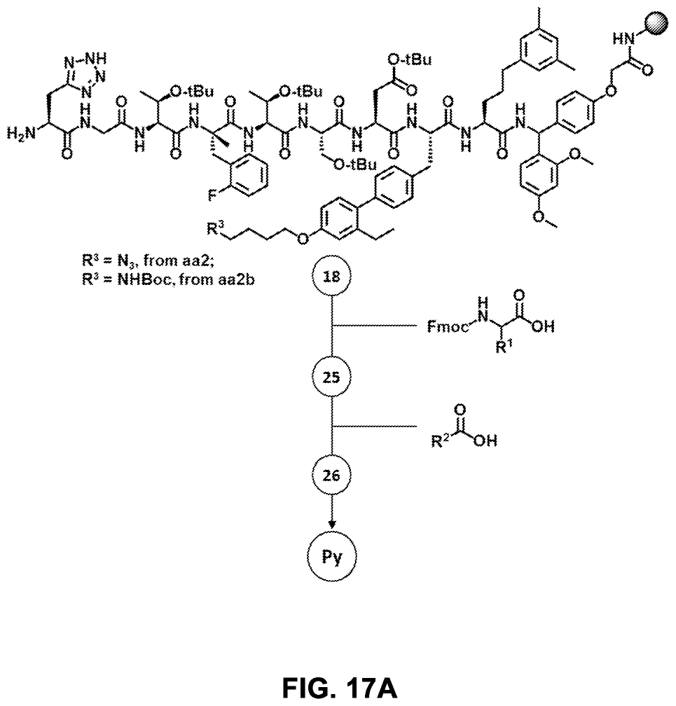

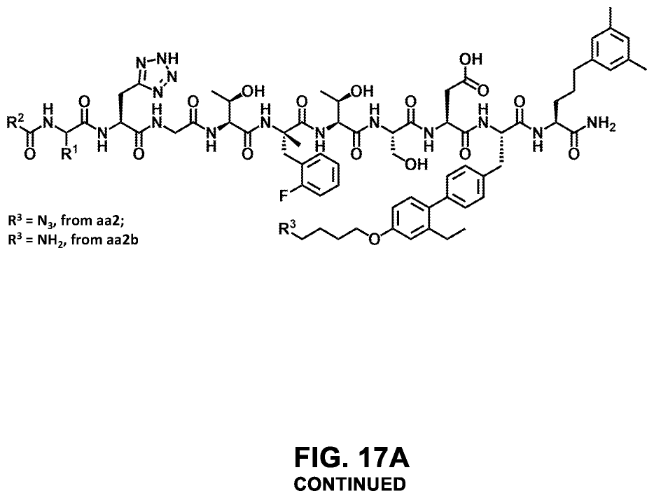

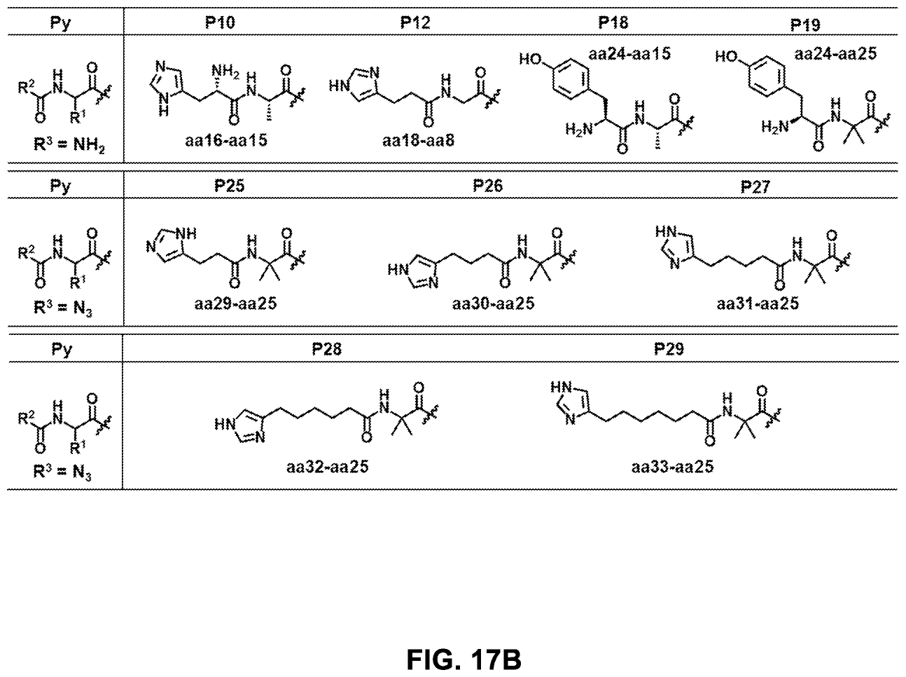

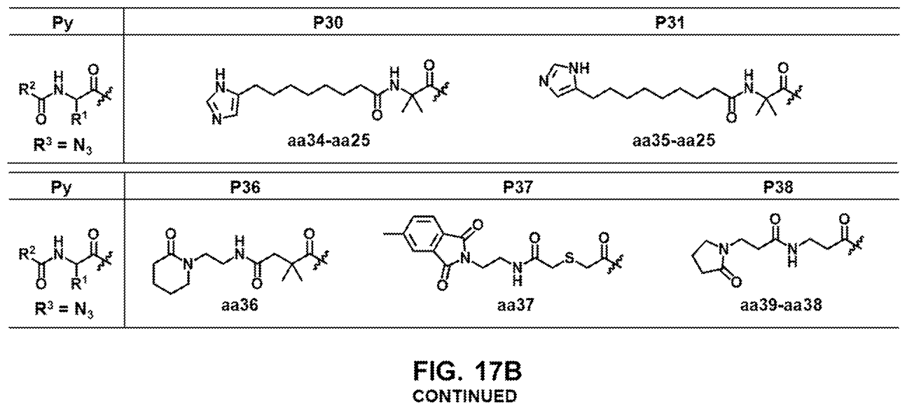

FIGS. 17A and 17B depict the sequence of steps for solid support synthesis of GLP1 peptidomimetic payloads P10, P12, P18, P19, P25, P26, P27, P28, P29, P30, P31, P36, P37, and P38 according to the disclosure.

### 3.16 Preparation of [[(2S)-2-[[(2S)-2-amino-3-(1H-imidazol-5-yl)propanoyl]amino]propanoyl]amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-oxo-butanoic acid (P10)

Starting from the corresponding aa9-aa8-aa7-aa6-aa5-aa4-aa3-aa2b-aa1 peptidyl Rink Amide MBHA Resin (18, 137.66 μmol), the corresponding aa16-aa15-aa9-aa8-aa7-aa6-aa5-aa4-aa3-aa2b-aa1 peptidyl Rink Amide MBHA Resin (26, 137.28 μmol) was prepared as described in the general procedure of SPPS by elongating the peptide with aa15 (128.58 mg, 412.99 μmol, 3 eq.) and then aa16 (155.42 mg, 411.83 μmol, 3 eq.).

The corresponding resin-bound peptide 26 (137.28 μmol) was further cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P10 (16 mg, 10.33 μmol, 7.55% yield, 99.85% purity) as a white solid.

LCMS (ESI): RT=0.808 min, mass calcd. for C74H100FN19017 1545.75 [M−4tBu-Boc+6H]+773.88 [M−4tBu-Boc+7H]2+, found 774.2 [M−4tBu-Boc+7H]2+. Reverse phase LC-MS was carried out using a Chromolith Flash RP-18e 25-3 mm column, with a flow rate of 1.5 mL/min, eluting with a gradient of 5% to 95% acetonitrile containing 0.04% TFA (solvent B) and water containing 0.06% TFA (solvent A).

HPLC: RT=7.46 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.17 Preparation of (3S)-4-[[(1S)-1-[[4-[4-(4-aminobutoxy)-2-ethyl-phenyl]phenyl]methyl]-2-[[(1S)-1-carbamoyl-4-(3-hydroxy-5-methyl-phenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[2-[3-(1H-imidazol-4-yl)propanoylamino]acetyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (P12)

Starting from the corresponding aa9-aa8-aa7-aa6-aa5-aa4-aa3-aa2b-aa1 peptidyl Rink Amide MBHA Resin (18, 82.60 μmol), the corresponding aa18-aa8-aa9-aa8-aa7-aa6-aa5-aa4-aa3-aa2b-aa1 peptidyl Rink Amide MBHA Resin (29, 82.60 μmol) was prepared as described in the general procedure of SPPS by elongating the peptide with aa8 (73.67 mg, 247.79 μmol, 3 eq.) and then aa18 (63.18 mg, 165.19 μmol, 2 eq.).

The corresponding resin-bound peptide 29 (82.60 μmol) was further cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P12 (11 mg, 7.09 μmol, 8.59% yield, 97.99% purity) as a white solid.

LCMS: (ESI): RT=0.828 min, mass calcd. for C72H96FN18018 759.86 m/z [M+2H]2+; found 759.7 m/z [M+2H]2+; [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=0.828 min, mass calcd. for C72H96FN18018 759.86 m/z [M+2H]2+; found 759.8 m/z [M+2H]2+; [M+2H]2+; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=7.65 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.18 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-2-[[(2S)-2-amino-3-hydroxy-propanoyl]amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-oxo-butanoic acid (P18)

Starting from the corresponding aa9-aa8-aa7-aa6-aa5-aa4-aa3-aa2b-aa1 peptidyl Rink Amide MBHA Resin compound 18 (76.48 μmol), the corresponding aa24-aa15-aa9-aa8-aa7-aa6-aa5-aa4-aa3-aa2b-aa1 peptidyl Rink Amide MBHA Resin compound 35 (76.48 μmol) was prepared as described in the general procedure of SPPS by elongating the peptide with aa15 (71.43 mg, 229.44 μmol, 3 eq.) and then aa24 (64.54 mg, 229.44 μmol, 3 eq.).

The corresponding resin-bound peptide 35 (76.48 μmol) was further cleaved following the general procedure to give the crude product as a white solid. The crude was purified by prep-HPLC (column: YMC-Exphere C18 10 μm 300*50 mm, 12 nm; mobile phase: [water (0.1% TFA)-ACN]; B %: 15%-45%, 55 min) to provide P18 (16 mg, 10.15 μmol, 11.76% yield, 99.77% purity) as a white solid.

LCMS: (ESI): RT=0.762 min, m/z calcd. for C77H104FN17018, 786.89 [M+2H]2+, m/z found 787.3 [M+2H]2+; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC (Rt=7.68 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 mL/min.

### 3.19 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-2-[[2-[[(2S)-2-amino-3-(4-hydroxyphenyl) propanoyl]amino]-2-methyl-propanoyl]amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxypropanoyl]amino]-4-oxo-butanoic acid (P19)

Starting from the corresponding aa9-aa8-aa7-aa6-aa5-aa4-aa3-aa2b-aa1 peptidyl Rink Amide MBHA Resin compound 18 (81.07 μmol), the corresponding aa24-aa25-aa9-aa8-aa7-aa6-aa5-aa4-aa3-aa2b-aa1 peptidyl Rink Amide MBHA Resin compound 37 (81.07 μmol) was prepared as described in the general procedure of SPPS by elongating the peptide with aa25 (79.13 mg, 243.20 μmol, 3 eq.) and then aa24 (68.41 mg, 243.20 μmol, 3 eq.).

The corresponding resin-bound peptide 37 was further cleaved following the general procedure to give the crude product as a white solid. The crude was purified by prep-HPLC (column: Phenomenex Gemini-NX 150*30 mm*5 μm; mobile phase: [water (0.04% NH₃H₂O+10 mM NH₄HCO₃)-ACN]; B %: 10%-50%, 15 min) to provide P19 (9 mg, 5.62 μmol, 7.03% yield, 99% purity) as a white solid.

LCMS: (ESI): RT=0.841 min, m/z calcd. for C₇₈H₁₀₆FN₁₇O₁₈, 793.90 [M+2H]²⁺, m/z found 794.2 [M+2H]²⁺; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

Crude HPLC: (Rt=7.81 min. Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

LCMS: (ESI): RT=0.813 min, m/z calcd. for C₇₈H₁₀₆FN₁₇O₁₈, 793.90 [M+2H]²⁺, m/z found 794.4 [M+2H]²⁺; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: Rt=7.81 min. Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.20 Preparation of (3S,6S,9S,12S,15S,21S)-21-((2H-tetrazol-5-yl)methyl)-3-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-28-(1H-imidazol-5-yl)-12,24,24-trimethyl-5,8,11,14,17,20,23,26-octaoxo-4,7,10,13,16,19,22,25-octaazaoctacosan-1-oic acid (P25)

Starting from the corresponding aa9-aa8-aa7-aa6-aa5-aa4-aa3-aa2b-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

To a mixture of Compound 36A (0.1 g, 50.70 μmol, 1.0 eq.) in DMF (20 mL) was added a solution of compound aa29 (58.17 mg, 152.11 μmol, 3.0 eq.), PyBOP (73.88 mg, 141.96 μmol, 2.8 eq.) and DIPEA (39.32 mg, 304.21 μmol, 52.99 μl, 6.0 eq) in DMF (20 mL) in one portion at 20° C. The mixture was bubbled with N2 at 20° C. for 2 hours. The mixture was filtered, and the collected resin was washed with DMF (50 mL*3), DCM (50 mL*3) to give the crude product on solid phase, which was subjected to acidic cleavage by using TFA cocktail (6 mL of TFA, 0.4 mL of water, 0.3 mL of triisopropylsilane, 120 mg of phenol). The mixture was filtered and the filtrate was diluted with t-BuOMe (1000 mL) to give a precipitate, which was centrifuged (5000 R) for 10 min. The residue was purified by prep-HPLC (column: Phenomenex Gemini-NX C18 80*30 mm*5 μm; mobile phase: [water (0.1% TFA)-ACN]; B %: 10%-60%, 20 min) to give the product P25 (2.3 mg, 1.33 μmol, 2.63% yield, 91% purity) as a white solid

LCMS (ESI): RT=4.006 min, m/z calcd. for C₇₅H₁₀₀FN₂₀O₁₇ 1571.76 [M+H]⁺, found 786.20 [M+2H]²⁺, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min. ESI source, Positive ion mode; Wavelength 220 nm&254 nm, Oven Temperature 50° C.

HPLC: RT=9.54 min, 98.48% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.21 Preparation of (3S,6S,9S,12S,15S,21S)-21-((2H-tetrazol-5-yl)methyl)-3-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-29-(1H-imidazol-5-yl)-12,24,24-trimethyl-5,8,11,14,17,20,23,26-octaoxo-4,7,10,13,16,19,22,25-octaazanonacosan-1-oic acid (P26)

Starting from the corresponding aa9-aa8-aa7-aa6-aa5-aa4-aa3-aa2-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

The Resin bound compound 36A (50 mg, 25.35 μmol, 1 eq.) was subjected to acidic cleavage by using TFA cocktail (5 mL, TFA/TIPS/H2O=95:2.5:2.5). The mixture was filtered, and the filtrate was diluted with t-BuOMe (50 mL) and then centrifuged (5000 R) for 10 min to give a crude product aa25-aa9-aa8-aa7-aa6-aa5-aa4-aa3-aa2-aa1 (50 mg, crude) as a white solid.

LCMS (ESI): RT=3.968 min, m/z calcd. for C69H95FN18016 1449.70, found 1449.7 [M+H]⁺, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min. ESI source, Positive ion mode; Wavelength 220 nm, 254 nm, Oven Temperature 50° C.

To a solution of aa30 (34.19 mg, 86.23 μmol, 2.5 eq.) in DMF (4 mL) was added PyBOP (39.49 mg, 75.88 μmol, 2.2 eq.) and DIPEA (26.75 mg, 206.96 μmol, 36.05 μL, 6 eq.), the mixture was stirred at 25° C. for 10 min, then aa25-aa9-aa8-aa7-aa6-aa5-aa4-aa3-aa2-aa1 (50 mg, 34.49 μmol, 1 eq.) was added, and the final mixture was stirred for 2 h at 25° C. The reaction progress was monitored by LCMS. After completion, the mixture was triturated by TBME (25 mL) to give a crude product, which was added into a solution of H2O (0.1 mL), triisopropylsilane (77.10 mg, 486.88 μmol, 0.1 mL, 14.83 eq.) and TFA (2.77 g, 24.31 mmol, 1.8 mL, 740.69 eq.). The mixture was stirred at 25° C. for 1 h. The reaction progress was monitored by LCMS. After completion, the mixture was filtered and then triturated by TBME (50 mL) to give a crude product, which was purified by prep-HPLC (column: Gemini NX C18 5 μm*10*150 mm; mobile phase: [water (0.1% TFA)-ACN]; B %: 10%-60%, 30 min) to give P26 (7.72 mg, 4.77 μmol, 14.54% yield, 98% purity) as a white solid.

LCMS (ESI): RT=3.991 min, mass calcd. for C₇₆H₁₀₃FN₂₀O₁₇, 793.38 [M+H]⁺, found 793.7 [M+H]⁺. LCMS conditions: 1.5 ML/4 L TFA in water (solvent A) and 0.75 ML/4 L TFA in acetonitrile (solvent B), using the elution gradient 10%-80% (solvent B) over 6.0 minutes and holding at 80% for 0.5 minutes at a flow rate of 0.8 ml/min; Column: X timate C18 2.1*30 mm, 3 μm; Wavelength: UV 220 nm & 254 nm Column temperature: 50° C.; MS ionization: ESI.

HPLC: RT=9.55 min, HPLC conditions: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: WELCH Ultimate LP-C18 150*4.6 mm 5 μm; Wavelength: UV 220 nm, 215 nm, 254 nm; Column temperature: 40° C.

### 3.22 Preparation of (3S,6S,9S,12S,15S,21S)-21-((2H-tetrazol-5-yl)methyl)-3-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-30-(1H-imidazol-5-yl)-12,24,24-trimethyl-5,8,11,14,17,20,23,26-octaoxo-4,7,10,13,16,19,22,25-octaazatriacontan-1-oic acid (P27)

Starting from the corresponding aa9-aa8-aa7-aa6-aa5-aa4-aa3-aa2-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

To a mixture of Compound 36A (120 mg, 60.84 μmol, 1 eq.) in DMF (4 mL) was added a solution of aa31 (62.44 mg, 152.11 μmol, 2.5 eq.), PyBOP (69.66 mg, 133.85 μmol, 2.2 eq.) and DIPEA (47.18 mg, 365.05 μmol, 63.59 μL, 6 eq.) in DMF (10 mL) in one portion at 20° C., and the final mixture was bubbled with N2 at 20° C. for 2 h and repeat this progress for twice. The reaction progress was monitored by LCMS. After completion, the mixture was filtered and washed with DMF (10 mL*4) and DCM (10 mL*4) to give the crude product on solid phase, which was subjected to acidic cleavage by using TFA cocktail (5 mL, TFA/TIPS/H2O=95:2.5:2.5). The mixture was filtered, and the filtrate was diluted with t-BuOMe (50 mL) to give a precipitate, which was centrifuged (5000 R) for 10 min. The residue was purified by prep-HPLC (column: Phenomenex Gemini NX C18 150*40 mm*5 μm; mobile phase: [water (0.1% TFA)-ACN]; B %: 0%-45%, 30 min) to give the product P27 (2.4 mg, 1.47 μmol, 2.48% yield, 98% purity) as a white solid

LCMS (ESI): RT=4.071 min, m/z calcd. for C₇₇H₁₀₅FN₂₀O₁₇ 800.39 [M+2H]²⁺, found 800.8, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min. ESI source, Positive ion mode; Wavelength 220 nm & 254 nm, Oven Temperature 50° C.

HPLC: RT=9.58 min, HPLC conditions: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: WELCH Ultimate LP-C18 150*4.6 mm 5 μm; Wavelength: UV 220 nm, 215 nm, 254 nm; Column temperature: 40° C.

### 3.23 Preparation of (3S,6S,9S,12S,15S,21S)-21-((2H-tetrazol-5-yl)methyl)-3-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-31-(1H-imidazol-4-yl)-12,24,24-trimethyl-5,8,11,14,17,20,23,26-octaoxo-4,7,10,13,16,19,22,25-octaazahentriacontan-1-oic acid (P28)

Starting from the corresponding aa9-aa8-aa7-aa6-aa5-aa4-aa3-aa2-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

To a mixture of Compound 36A (120 mg, 60.84 μmol, 1 eq.) in DMF (4 mL) was added a solution of aa32 (64.57 mg, 152.10 μmol, 2.5 eq.), PyBOP (69.65 mg, 133.85 μmol, 2.2 eq.) and DIPEA (39.32 mg, 304.20 μmol, 52.99 μL, 5 eq.) in DMF (10 mL) in one portion at 20° C., and the final mixture was bubbled with N2 at 20° C. for 2 h and repeat this progress for twice. The reaction progress was monitored by LCMS. After completion, the mixture was filtered and washed with DMF (10 mL*4) and DCM (10 mL*4) to give the crude product on solid phase, which was subjected to acidic cleavage by using TFA cocktail (5 mL, TFA/TIPS/H2O=95:2.5:2.5). The mixture was filtered, and the filtrate was diluted with t-BuOMe (50 mL) to give a precipitate, which was centrifuged (5000 R) for 10 min. The residue was purified by prep-HPLC (column: Phenomenex Gemini-NX 150*30 mm*5 μm; mobile phase: [water (0.05% ammonia hydroxide v/v)-ACN]; B %: 0%-45%, 30 min) to give the product P28 (8.5 mg, 5.21 μmol, 10.34% yield, 99% purity) as a white solid

LCMS (ESI): RT=4.035 min, m/z calcd. for C₇₈H₁₀₇FN₂₀O₁₇ 807.40 [M+2H]²⁺, found 807.8, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min. ESI source, Positive ion mode; Wavelength 220 nm & 254 nm, Oven Temperature 50° C.

HPLC: RT=9.60 min, HPLC conditions: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: WELCH Ultimate LP-C18 150*4.6 mm 5 μm; Wavelength: UV 220 nm, 215 nm, 254 nm; Column temperature: 40° C.

### 3.24 Preparation of (3S,6S,9S,12S,15S,21S)-21-((2H-tetrazol-5-yl)methyl)-3-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-32-(1H-imidazol-4-yl)-12,24,24-trimethyl-5,8,11,14,17,20,23,26-octaoxo-4,7,10,13,16,19,22,25-octaazadotriacontan-1-oic acid (P29)

Starting from the corresponding aa9-aa8-aa7-aa6-aa5-aa4-aa3-aa2-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

To a mixture of Compound 36A (120 mg, 60.84 μmol, 1 eq.) in DMF (4 mL) was added a solution of aa33 (64.04 mg, 146.02 μmol, 2.4 eq.), PyBOP (63.32 mg, 121.68 μmol, 2 eq.) and DIPEA (39.32 mg, 304.21 μmol, 52.99 μL, 5 eq.) in DMF (10 mL) in one portion at 20° C., and the final mixture was bubbled with N2 at 20° C. for 2 h and repeat this progress for twice. The reaction progress was monitored by LCMS. After completion, the mixture was filtered and washed with DMF (10 mL*4) and DCM (10 mL*4) to give the crude product on solid phase, which was subjected to acidic cleavage by using TFA cocktail (5 mL, TFA/TIPS/H2O=95:2.5:2.5). The mixture was filtered, and the filtrate was diluted with t-BuOMe (50 mL) to give a precipitate, which was centrifuged (5000 R) for 10 min. The residue was purified by prep-HPLC (column: Phenomenex Gemini-NX 150*30 mm*5 μm; mobile phase: [water (0.1% TFA)-ACN]; B %: 15%-55%, 20 min) to give the product P29 (5 mg, 3.05 μmol, 5.22% yield, 99.4% purity) as a white solid

LCMS (ESI): RT=3.997 min, m/z calcd. for C₇₇H₁₀₃FN₂₀O₁₇, 814.40 [M+2H]²⁺, found 814.9, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min. ESI source, Positive ion mode; Wavelength 220 nm & 254 nm, Oven Temperature 50° C.

HPLC: RT=9.61 min, HPLC conditions: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: WELCH Ultimate LP-C18 150*4.6 mm 5 μm; Wavelength: UV 220 nm, 215 nm, 254 nm; Column temperature: 40° C.

### 3.25 Preparation of (3S)-4-[[(1S)-1-[[4-[4-(4-azidobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[2-[8-(1H-imidazol-5-yl) octanoylamino]-2-methyl-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P30)

Starting from the corresponding aa9-aa8-aa7-aa6-aa5-aa4-aa3-aa2-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

To a mixture of Compound 36A (500 mg, 126.75 μmol, 50% purity, 1 eq) in DMF (4 mL) was added a solution of aa34 (286.84 mg, 633.77 μmol, 5 eq), HATU (86.75 mg, 228.16 μmol, 1.8 eq) and DIPEA (65.53 mg, 507.02 μmol, 88.31 μL, 4 eq) in DMF (20 mL) in one portion at 20° C., and the final mixture was bubbled with N2 at 20° C. for 2 h. The reaction progress was monitored by LCMS. After completion, the mixture was filtered and washed with DMF (10 mL*4) and DCM (10 mL*4) to give the crude product on solid phase, which was subjected to acidic cleavage by using TFA cocktail (10 mL, TFA/TIPS/H2O=95:2.5:2.5). The mixture was filtered, and the filtrate was diluted with t-BuOMe (100 mL) to give a precipitate, which was centrifuged (5000 R) for 10 min. The residue was purified by prep-HPLC (column: Boston Green ODS 150*30 mm*5 μm; mobile phase: [water (0.1% TFA)-ACN]; B %: 22%-62%, 9 min) to give the product P30 (7.68 mg, 4.62 μmol, 4.45% yield, 98.82% purity) as a white solid

LCMS (ESI): RT=3.998 min, m/z calcd. for C₃₀H₁₁₀FN₂₀O₁₇ 1641.83 [M+H]⁺, C₈₀H₁₁₁FN₂₀O₁₇ 821.4 [M+2H]²⁺, found 821.8 [M+2H]2+. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=9.63 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: YMC-Pack ODS-A 150*4.6 mm, 5 μm.

### 3.26 Preparation of (3S)-4-[[(1S)-1-[[4-[4-(4-azidobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[2-[9-(1H-imidazol-5-yl)propanoyl]amino]-2-methyl-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P31)

Starting from the corresponding aa9-aa8-aa7-aa6-aa5-aa4-aa3-aa2-aa1 peptidyl Rink Amide MBHA Resin (1.03 mmol), aa25 (669 mg, 206 mmol, 2.0 eq.), the corresponding aa25-aa9-aa8-aa7-aa6-aa5-aa4-aa3-aa2-aa1 peptidyl Rink Amide MBHA Resin compound 36A (1.03 mmol) was prepared as described in the general procedure of SPPS.

To a mixture of Compound 36A (125 mg, 63.38 μmol, 1 eq.) in DMF (4 mL) was added a solution of aa35 (57.37 mg, 126.75 μmol, 2.0 eq.), HATU (43.38 mg, 114.08 μmol, 1.8 eq.) and DIPEA (32.76 mg, 253.51 μmol, 44.16 μl, 4.0 eq.) in DMF (10 mL) in one portion at 20° C., and the final mixture was bubbled with N2 at 20° C. for 2 h. The reaction progress was monitored by LCMS. After completion, the mixture was filtered and washed with DMF (10 mL*4) and DCM (10 mL*4) to give the crude product on solid phase, which was subjected to acidic cleavage by using TFA cocktail (5 mL, TFA/TIPS/H2O=95:2.5:2.5). The mixture was filtered, and the filtrate was diluted with t-BuOMe (50 mL) to give a precipitate, which was centrifuged (5000 R) for 10 min. The residue was purified by prep-HPLC (column: Boston Green ODS 150*30 mm*5 μm; mobile phase: [water (0.1% TFA)-ACN]; B %: 25%-65%, 9 min) to give the product P31 (7.0 mg, 4.23 μmol, 6.69% yield, 100% purity) as a white solid.

LCMS (ESI): RT=4.023 min, m/z calcd. for C₇₅H₁₀₀FN₂₀O₁₇ 1571.76 [M+H]⁺, 786.38 [M+2H]²⁺, found 786.20 [M+2H]²⁺, Mobile Phase: 1.5 ML/4 L TFA in water (solvent A) and 0.75 ML/4 L TFA in acetonitrile (solvent B), using the gradient 10%-80% (solvent B) over 2.5 minutes and holding at 80% for 0.5 minutes at a flow rate of 0.8 ml/min. ESI source, Positive ion mode; Wavelength 220 nm, 254 nm, OvenTemperature 50° C.

HPLC: RT=9.80 min, 100% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.27 Preparation of (9S,15S,18S,21S,24S,27S)-9-((2H-tetrazol-5-yl)methyl)-27-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-18-(2-fluorobenzyl)-15,21-bis((R)-1-hydroxyethyl)-24-(hydroxymethyl)-6,6,18-trimethyl-4,7,10,13,16,19,22,25-octaoxo-1-(2-oxopiperidin-1-yl)-3,8,11,14,17,20,23,26-octaazanonacosan-29-oic acid (P36)

To a mixture of aa9-aa8-aa7-aa6-aa5-aa4-aa3-aa2-aa1 peptidyl Rink Amide MBHA Resin (75 mg, 19.87 μmol, 50% purity, 1 eq) in DMF (2 mL) was added a solution of aa36 (20 mg, 73.99 μmol, 3.72 eq), HATU (15.11 mg, 39.74 μmol, 2 eq) and DIPEA (25.68 mg, 198.71 μmol, 34.61 μL, 10 eq) in DMF (10 mL) in one portion at 20° C., and the final mixture was bubbled with N2 at 20° C. for 2 h. The reaction progress was monitored by LCMS. After completion, the mixture was filtered and washed with DMF (10 mL×4) and DCM (10 mL×4) to give the crude product on solid phase, which was subjected to acidic cleavage by using TFA cocktail (5 mL, TFA/TIPS/H2O=95:2.5:2.5). The mixture was filtered, and the filtrate was diluted with t-BuOMe (50 mL) to give a precipitate, which was centrifuged (5000 R) for 10 min. The residue was purified by prep-HPLC (column: Welch Xtimate C18 100*40 mm*3 μm; mobile phase: [water(0.075% TFA)-ACN]; B %: 50%-80%, 10 min) to give the product P36 (2.4 mg, 1.48 μmol, 7.47% yield, 100% purity) as a white solid.

LCMS (ESI): RT=4.416 min, mass calcd. for C₉₅H₁₂₂N₂₀O₂₂FH 1917.11 [M+H]⁺, C₉₅H₁₂₂N₂₀O₂₂F 809.1 [M+2H]²⁺, found 809.3 [M+2H]2+; Reverse phase LCMS was carried out using a Chromolith Flash column 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate 3 μm, C18, 2.1*30 mm.

HPLC: RT=5.24 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: Ultimate XB-C18, 3 μm, 3.0*50 mm.

### 3.28 Preparation of (3S)-4-[[(1S)-1-[[4-[4-(4-azidobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[2-[2-[2-(5-methyl-1,3-dioxo-isoindolin-2-yl)ethylamino]-2-oxo-ethyl]sulfanylacetyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P37)

To a mixture of aa9-aa8-aa7-aa6-aa5-aa4-aa3-aa2-aa1 peptidyl Rink Amide MBHA Resin (100 mg, 26.49 μmol, 1 eq) in DMF (2 mL) was added a solution of aa36 (44.56 mg, 132.47 μmol, 5 eq), HATU (18.13 mg, 47.69 μmol, 1.8 eq) and DIPEA (13.70 mg, 105.98 μmol, 18.46 μL, 4 eq) in DMF (10 mL) in one portion at 20° C., and the final mixture was bubbled with N2 at 20° C. for 2 h. The reaction progress was monitored by LCMS. After completion, the mixture was filtered and washed with DMF (10 mL×4) and DCM (10 mL×4) to give the crude product on solid phase, which was subjected to acidic cleavage by using TFA cocktail (5 mL, TFA/TIPS/H2O=95:2.5:2.5). The mixture was filtered, and the filtrate was diluted with t-BuOMe (50 mL) to give a precipitate, which was centrifuged (5000 R) for 10 min. The residue was purified by prep-HPLC (column: Waters Xbridge BEH C18 100*25 mm*5 μm; mobile phase: [water(0.1% TFA)-ACN]; B %: 20%-80%, 15 min) to give the product P37 (4.81 mg, 2.86 μmol, 10.86% yield, 100% purity) as a white solid.

LCMS (ESI): RT=4.602 min, m/z calcd. for C₃₀H₁₀₁FN₁₉O₁₉S 1682.71 [M+H]⁺, C₃₀H₁₀₀FN₁₉O₁₉S 841.85 [M+2H]²⁺, found 842.3 [M+2H]²⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=4.844 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm.

### 3.29 Preparation of (3S,6S,9S,12S,15S,21S)-21-((2H-tetrazol-5-yl)methyl)-3-(((S)-1-(((S)-1-amino-5-(3,5-dimethylphenyl)-1-oxopentan-2-yl)amino)-3-(4′-(4-azidobutoxy)-2′-ethyl-[1,1′-biphenyl]-4-yl)-1-oxopropan-2-yl)carbamoyl)-12-(2-fluorobenzyl)-9,15-bis((R)-1-hydroxyethyl)-6-(hydroxymethyl)-12-methyl-5,8,11,14,17,20,23,27-octaoxo-29-(2-oxopyrrolidin-1-yl)-4,7,10,13,16,19,22,26-octaazanonacosan-1-oic acid (P38)

To a mixture of aa9-aa8-aa7-aa6-aa5-aa4-aa3-aa2-aa1 peptidyl Rink Amide MBHA Resin (0.2 g, 52.99 μmol, 50% purity, 1.0 eq.) in DMF (2 mL) was added a solution of aa38 (82.48 mg, 264.94 μmol, 5.0 eq.), HATU (90.66 mg, 238.45 μmol, 4.5 eq.) and DIPEA (68.48 mg, 529.88 μmol, 92.29 μL, 10.0 eq.) in DMF (10 mL) in one portion at 20° C., and the final mixture was bubbled with N2 at 20° C. for 2 h. The reaction progress was monitored by LCMS. After completion, the mixture was filtered and washed with DMF (10 mL×4) and DCM (10 mL×4) to give the crude product on solid phase, which was swelled again with 20% piperidine/DMF (20 mL) and bubbled with N2 at 20° C. for 2 hr. After completion, the mixture was filtered, and the collected resin was washed with DMF (100 mL×3), DCM (100 mL×3) to give the crude product aa38-aa9-aa8-aa7-aa6-aa5-aa4-aa3-aa2-aa1 on solid phase (52.99 μmol).

To a mixture of aa38-aa9-aa8-aa7-aa6-aa5-aa4-aa3-aa2-aa1 peptidyl Rink Amide MBHA Resin (52.60 μmol, 1 eq.) in DMF (2 mL) was added a solution of aa39 (41.33 mg, 262.98 μmol, 5.0 eq.), HATU (90.00 mg, 236.69 μmol, 4.5 eq.) and DIPEA (67.98 mg, 525.97 μmol, 91.61 μL, 10.0 eq.) in DMF (10 mL) in one portion at 20° C., and the final mixture was bubbled with N2 at 20° C. for 2 h. The reaction progress was monitored by LCMS. After completion, the mixture was filtered and washed with DMF (10 mL×4) and DCM (10 mL×4) to give the crude product on solid phase, which was subjected to acidic cleavage by using TFA cocktail (10 mL, TFA/TIPS/H2O=95:2.5:2.5). The mixture was filtered, and the filtrate was diluted with t-BuOMe (100 mL) to give a precipitate, which was centrifuged (5000 R) for 10 min. The residue was purified by prep-HPLC (column: Boston Green ODS 150*30 mm*5 μm; mobile phase: [water (0.1% TFA)-ACN]; B %: 36%-76%, 9 min) and prep-HPLC (column: Waters X-bridge BEH C18 100*25 mm*5 μm; mobile phase: [water (0.05% NH₃H2O)-ACN]; B %: 5%-49%, 11 min) to give the product P38 (3.0 mg, 1.71 μmol, 3.26% yield, 90% purity) as a white solid. LCMS (ESI): RT=4.313 min, m/z calcd. for C₇₅H₁₀₂FN₁₉O₁₈ 1575.76 [M+H]⁺, 788.38 [M+2H]²⁺, found 788.20 [M+2H]²⁺. LCMS conditions: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate 3 μm, C18,2.1*30 mm; HPLC: RT=9.93 min, 90% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

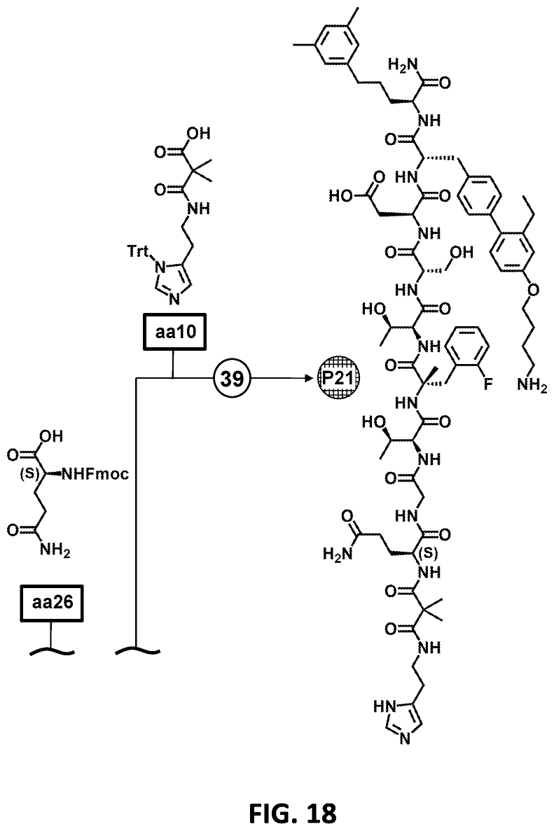

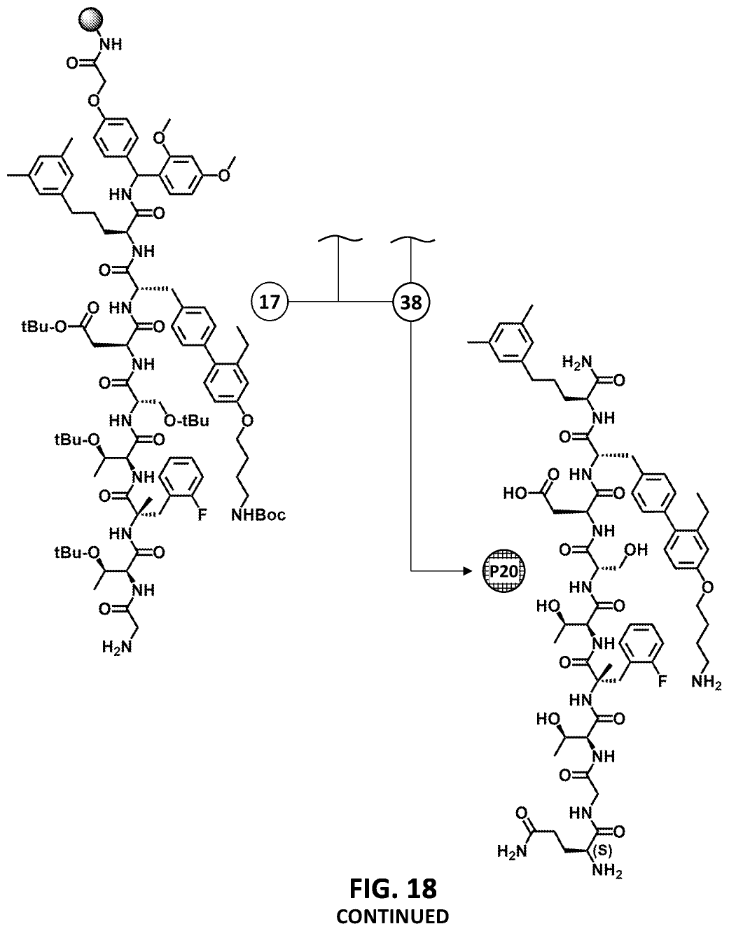

FIG. 18 depicts the sequence of steps for solid support synthesis of GLP1 peptidomimetic payloads P20 and P21 according to the disclosure.

### 3.30 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-2,5-diamino-5-oxopentanoyl]amino]acetyl]amino]-3-hydroxy-butanoyl]amino]-3-(2-fluorophenyl)-2-methyl-propanoyl]amino]-3-hydroxybutanoyl]amino]-3-hydroxy-propanoyl]amino]-4-oxo-butanoic acid (P20)

Starting from the corresponding aa8-aa7-aa6-aa5-aa4-aa3-aa2b-aa1 peptidyl Rink Amide MBHA Resin (17, 175.17 μmol) and aa26 (193.59 mg, 525.51 μmol, 3 eq.), the corresponding aa26-aa8-aa7-aa6-aa5-aa4-aa3-aa2b-aa1 peptidyl Rink Amide MBHA Resin (38, 175.17 μmol) was prepared as described in the general procedure of SPPS.

The corresponding resin-bound peptide 38 (175.17 μmol) was further cleaved following the general procedure to give the crude product as a white solid. The crude was purified by prep-HPLC (column: Waters Xbridge Prep OBD C18 150*40 mm*10 μm; mobile phase: [water (10 mM NH₄HCO₃)-ACN]; B %: 0%-90%, 25 min) to provide P20 (4.65 mg, 3.35 μmol, 95.77% purity) as a white solid.

LCMS: (ESI): RT=0.789 min, mass calcd. for C₆₆H₉₃FN₁₂O₁₆ 664.34 m/z [M+2H]²⁺; found 665.0 m/z [M+2H]²⁺; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

LCMS: (ESI): RT=1.362 min, mass calcd. for C₆₆H₉₂FN₁₂O₁₆ 1327.67 m/z [M+H]⁺; found 1327.7 m/z [M+H]⁺; LC-MS: Mobile Phase: 1.5 ML/4 L TFA in water (solvent A) and 0.75 ML/4 L TFA in acetonitrile (solvent B),using the gradient 10%-80% (solvent B) over 2 minutes and holding at 80% for 0.48 minutes at a flow rate of 0.8 ml/min.

HPLC: RT=7.40 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

### 3.31 Preparation of (3S)-4-[[(1S)-1-[[4-[4-(4-aminobutoxy)-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-2-[[(2S,3R)-2-[[2-[[(2S)-5-amino-2-[[3-[2-(1H-imidazol-5-yl)ethylamino]-2,2-dimethyl-3-oxo-propanoyl]amino]-5-oxo-pentanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-oxo-butanoic acid (P21)

Starting from the corresponding aa26-aa8-aa7-aa6-aa5-aa4-aa3-aa2b-aa1 peptidyl Rink Amide MBHA Resin compound 38 (175.17 μmol) and aa10 (163.80 mg, 350.34 μmol, 2 eq.), the corresponding aa10-aa26-aa8-aa7-aa6-aa5-aa4-aa3-aa2b-aa1 peptidyl Rink Amide MBHA Resin (39, 175.17 μmol) was prepared as described in the general procedure of SPPS.

The corresponding resin-bound peptide 39 (175.17 μmol) was further cleaved following the general procedure to give the crude product as a white solid. The crude was purified by prep-HPLC (column: Waters Xbridge Prep OBD C18 150*40 mm*10 μm; mobile phase: [water (0.1% TFA)-ACN]; B %: 15%-45%, 20 min) to provide P21 (20 mg, 12.94 μmol, 7.39% yield, 99.31% purity) as a white solid.

LCMS: (ESI): RT=0.833 min, mass calcd. for C₇₆H₁₀₆FN₁₅O₁₈ 767.89 m/z [M+2H]²⁺; found 768.3 m/z [M+2H]²⁺; LC-MS: MERCK, RP-18e 25-2 mm column, flow rate 1.5 mL/min, eluting with a gradient of 5% to 95% acetonitrile containing 0.02% TFA (solvent B) and water containing 0.04% TFA (solvent A).

HPLC: RT=7.44 min. Mobile Phase: 2.75 ML/4LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature: 40° C.

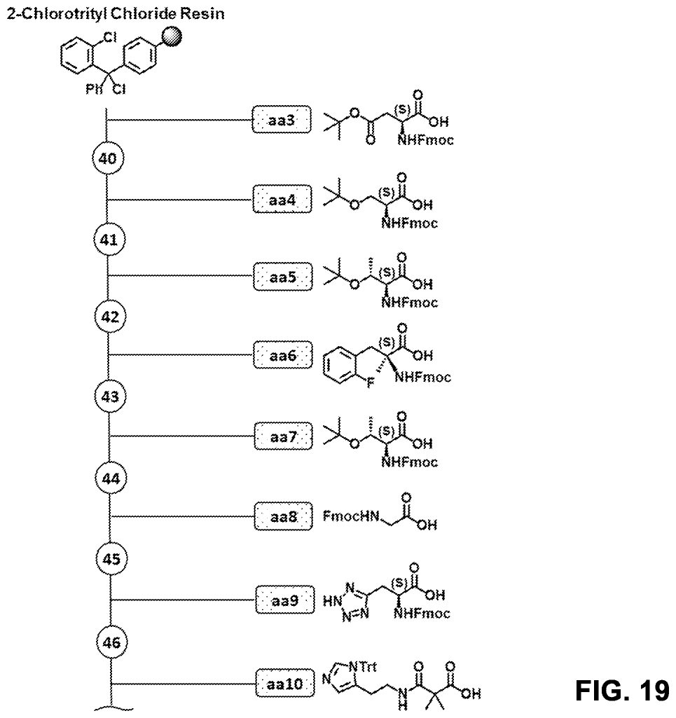

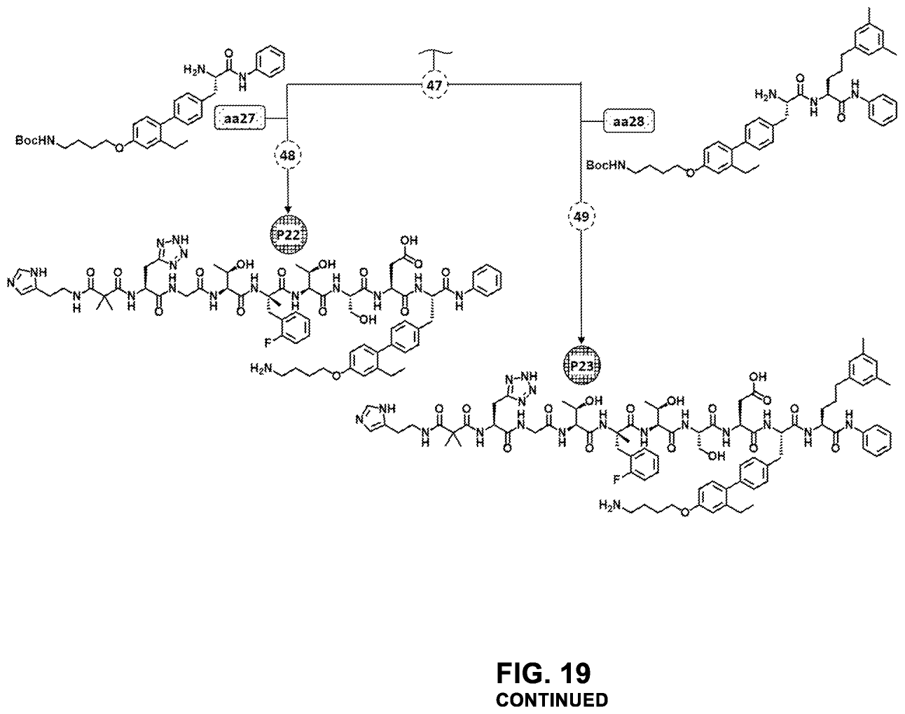

FIG. 19 depicts the sequence of steps for solid support synthesis of GLP1 peptidomimetic payloads P22 and P23 according to the disclosure.

### 3.32 Preparation of (3S)-4-[[(1S)-1-[[4-[4-(4-aminobutoxy)-2-ethyl-phenyl]phenyl]methyl]-2-anilino-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P22)

The corresponding aa10-aa9-aa8-aa7-aa6-aa5-aa4-aa3 peptidyl 2-Chlorotrityl Chloride Resin bound 47 was prepared as described in the general procedure of SPPS.

To a mixture of the corresponding resin-bound peptide (47, 553.98 μmol) was added TFE (2.61 g, 26.09 mmol, 1.88 mL, 47.09 eq.) and AcOH (1.97 g, 32.83 mmol, 1.88 mL, 59.26 eq.) in DCM (8 mL) in one portion at 25° C. under N₂. The mixture was shaked at 25° C. for 2 hours. LCMS trace showed that the reaction was complete. The mixture was filtered, and the cake was washed with DCM (5 mL×3). The filtrate was concentrated in vacuum to give a yellow oil, which was diluted with water (5 mL). The mixture was adjusted to pH=8 with aq. sat. NaHCO₃, yellow solids were precipitated. The mixture was filtered, and the cake was washed with water (5 mL×2), dried in vacuum to give crude product (550 mg, crude) as a yellow solid. The crude was purified by prep-HPLC (column: Xtimate C18 150*25 mm*5 μm; mobile phase: [water (0.225% FA)-ACN]; B %: 48%-58%, 11 min) to give compound 47 (220 mg, 122.65 μmol, 36.10% yield, 82.05% purity) as an off-white solid.

LCMS (ESI): RT=1.073 min, mass calcd. for C₇₆H₁₀₄FN₁₄O₁₅ 1471.78 m/z [M−C₁₉H₁₃Cl+2H]⁺, m/z found 1471.65; [M-C₁₉H₁₃Cl+2H]⁺, rink 2-[chloro(diphenyl)methyl]benzene (C₁₉H₁₃Cl, exact mass=276.1); Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

LCMS (ESI): RT=1.079 min, mass calcd. for C₇₆H₁₀₄FN₁₄O₁₅ 1471.78 m/z [M−C₁₉H₁₃Cl+2H]⁺, m/z found 1471.8; [M−C₁₉H₁₃Cl+2H]⁺, rink 2-[chloro(diphenyl)methyl]benzene (C₁₉H₁₃Cl, exact mass=276.1); Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC: RT=10.96 min. HPLC: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min

To a solution of compound 47 (37.37 μmol) and HOBt (5.05 mg, 37.37 μmol, 1 eq.) in CHCl₃ (0.225 mL) and DMF (0.025 mL) was added aa27 (19.87 mg, 37.37 μmol, 1 eq.) at 20° C. A solution of DIC (4.72 mg, 37.37 μmol, 5.79 μL, 1 eq.) in CHCl₃ (0.225 mL) and DMF (0.025 mL) were added to the mixture. The reaction was stirred at 20° C. for 16 hours. LCMS trace showed that the reaction was complete. The reaction was concentrated in vacuum to give crude product 48 (74.2 mg, 37.37 μmol) as a brown oil, which was used to the next step without further purification.

LCMS (ESI): RT=1.215 min, mass calcd. for C₁₀₃H₁₄₄FN₁₇O₁₃ 993.05 m/z [M+2H]²⁺, m/z found 993.8 m/z [M+2H]²⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

To a mixture of compound 48 (74.2 mg, 37.37 μmol, 1 eq.) was added triisopropylsilane (137.30 mg, 867.01 μmol, 178.08 μL, 23.20 eq.) in TFA (2 mL) and H₂O (0.06 mL) in one portion at 25° C. under N₂. The mixture was standing at 15° C. for 2.5 hours. LCMS trace showed that the reaction was complete. The mixture was concentrated in vacuum to give crude as a yellow oil. The crude was purified by prep-HPLC (column: Waters Xbridge Prep OBD C18 150*30 mm, 10 μm; mobile phase: [water (10 mM NH₄HCO₃)-ACN]; B %: 28%-58%, 11 min) to give compound P22 (7.5 mg, 5.18 μmol, 13.86% yield, 97.96% purity) as a white solid.

LCMS (ESI): RT=0.811 min, mass calcd. for C₆₈H₉₀FN₁₇O₁₆ 709.84 m/z [M+2H]²⁺, m/z found 710.2 m/z [M+2H]²⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC: RT=7.06 min. HPLC: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min.

### 3.33 Preparation of (3S)-4-[[(1S)-1-[[4-[4-(4-aminobutoxy)-2-ethyl-phenyl]phenyl]methyl]-2-[[(1S)-4-(3,5-dimethylphenyl)-1-(phenylcarbamoyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P23)

To a solution of 47 (27.18 μmol) and HOBt (3.67 mg, 27.18 μmol, 1 eq.) in CHCl₃ (0.225 mL) and DMF (0.025 mL) was added aa28 (19.98 mg, 27.18 μmol, 1 eq.) at 20° C. A solution of DIC (3.43 mg, 27.18 μmol, 4.21 μL, 1 eq.) in CHCl₃ (0.225 mL) and DMF (0.025 mL) were added to the mixture. The reaction was stirred at 20° C. for 16 hours. LCMS trace showed that the reaction was complete. The reaction was concentrated in vacuum to give crude product 49 (59.49 mg, 27.18 μmol) as a brown oil, which was used to the next step without further purification.

LCMS (ESI): RT=1.223 min, mass calcd. for C₁₁₆H₁₅₄FN₁₈O₁₇ 1045.09 m/z [M−Boc+3H]²⁺, m/z found 1044.8 m/z [M-Boc+2H]²⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

To a mixture of compound 49 (59.49 mg, 27.18 μmol, 1 eq.) was added triisopropylsilane (131.07 mg, 827.67 μmol, 0.17 mL, 30.45 eq.) in TFA (6 mL) and H₂O (0.17 mL) in one portion at 15° C. under N₂. The mixture was standing at 15° C. for 2.5 hours. LCMS trace showed that the reaction was complete. The mixture was concentrated in vacuum to give crude as yellow oil. The crude was purified by prep-HPLC (column: Xtimate C18 10 μm, 250 mm*50 mm; mobile phase: [water (0.04% NH₃H2O+10 mM NH4HCO3)-ACN]; B %: 25%-55%, 8 min) to give compound P23 (7.5 mg, 4.60 μmol, 16.91% yield, 99.38% purity) as a white solid.

LCMS (ESI): RT=0.875 min, mass calcd. for C₈₁H₁₀₇FN₁₈O₁₇ 811.4 m/z [M+2H]²⁺, m/z found 811.9 m/z [M+2H]²⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

LCMS (ESI): RT=0.882 min, mass calcd. for C₈₁H₁₀₇FN₁₈O₁₇ 811.4 m/z [M+2H]²⁺, m/z found 811.8 m/z [M+2H]²⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC: RT=8.30 min. HPLC: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

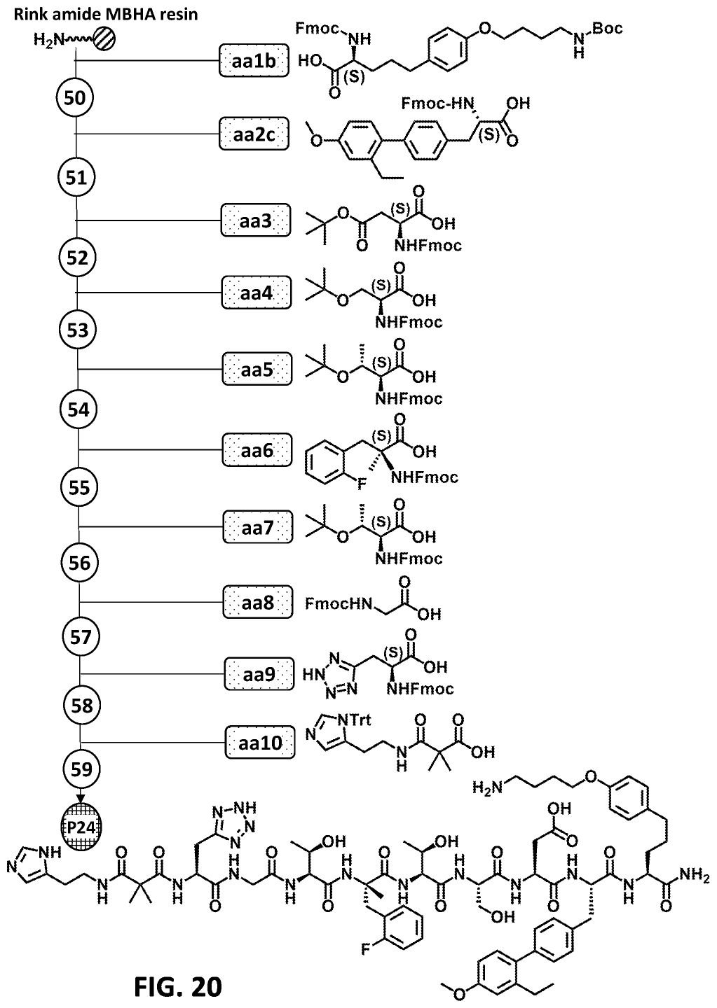

FIG. 20 depicts the sequence of steps for solid support synthesis of GLP1 peptidomimetic payload P24 according to the disclosure.

### 3.34 Preparation of (3S)-4-[[(1S)-2-[[(1S)-4-[4-(4-aminobutoxy)phenyl]-1-carbamoyl-butyl]amino]-1-[[4-(2-ethyl-4-methoxy-phenyl)phenyl]methyl]-2-oxo-ethyl]amino]-3-[[(2S)-2-[[(2R,3R)-2-[[(2S)-3-(2-fluorophenyl)-2-[[(2S,3R)-3-hydroxy-2-[[2-[[(2S)-2-[[3-[2-(1H-imidazol-5-yl) ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (P24)

The corresponding aa10-aa9-aa8-aa7-aa6-aa5-aa4-aa3-aa2c-aa1b peptidyl Rink Amide MBHA Resin (59) was prepared as described in the general procedure of SPPS.

The corresponding resin-bound peptide 59 was further cleaved following the general procedure to give the crude product as a white solid. The crude product was purified by prep-HPLC (column: mobile phase: [water (10 mM NH₄HCO₃)-ACN]; B %: 15%-45%, 55 min) to afford pure product. The product was suspended in water (20 mL), the mixture frozen in a dry-ice/acetone bath, and then lyophilized to dryness to afford the desired product. Compound P24 (50 mg, 29.69 μmol, 6.76% yield, 98.686% purity, TFA salt) was obtained as a white solid.

LCMS (ESI): RT=0.821 min, mass calcd. for C₇₄H₉₉FN₁₈O₁₈ 1546.74 [M+H]⁺, 773.4 [M+H]²⁺, m/z found 774.8 [M+H]²⁺. Reverse phase LC-MS was carried out using a Chromolith Flash RP-18e 25-2 mm column, with a flow rate of 1.5 mL/min, eluting with a gradient of 5% to 95% acetonitrile containing 0.02% TFA (solvent B) and water containing 0.04% TFA (solvent A).

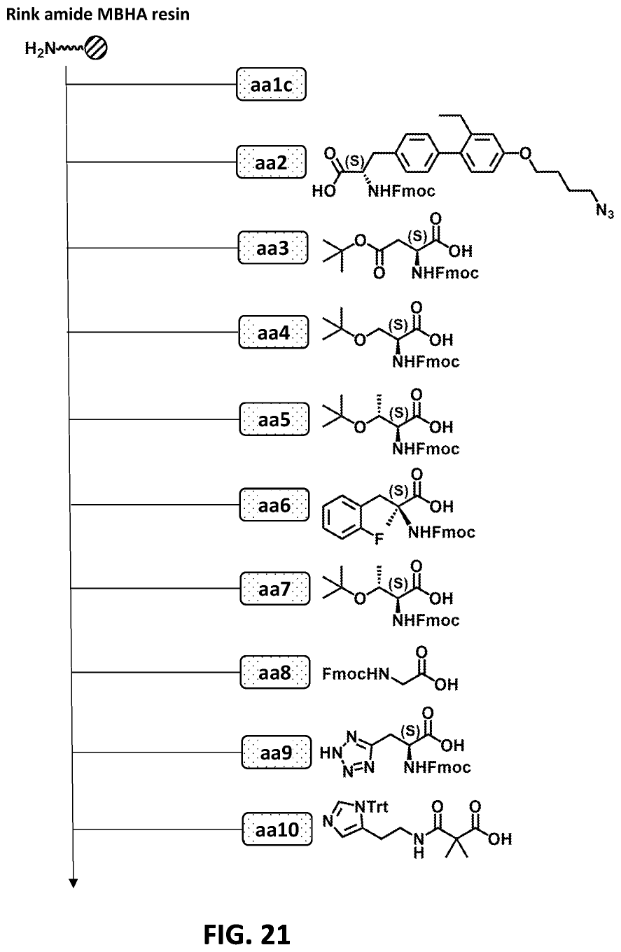

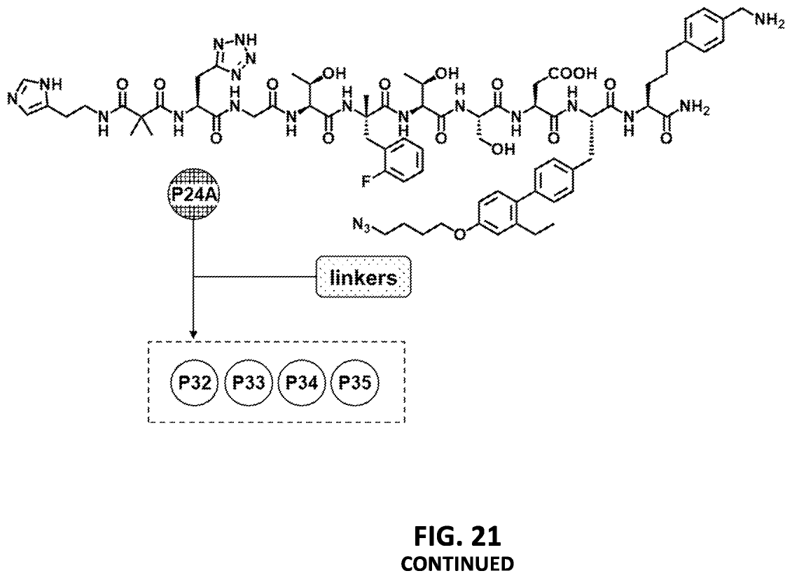

FIG. 21 depicts the sequence of steps for solid support synthesis of GLP1 peptidomimetic payloads P32, P33, P34, and P35 according to the disclosure.

### 3.35 Preparation of (8S,14S,17S,20S,23S,26S)-8-((2H-tetrazol-5-yl)methyl)-26-(((S)-1-(((S)-1-amino-5-(4-(aminomethyl)phenyl)-1-oxopentan-2-yl)amino)-3-(4′-(4-azidobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P24A) (SEQ ID NO: 600)

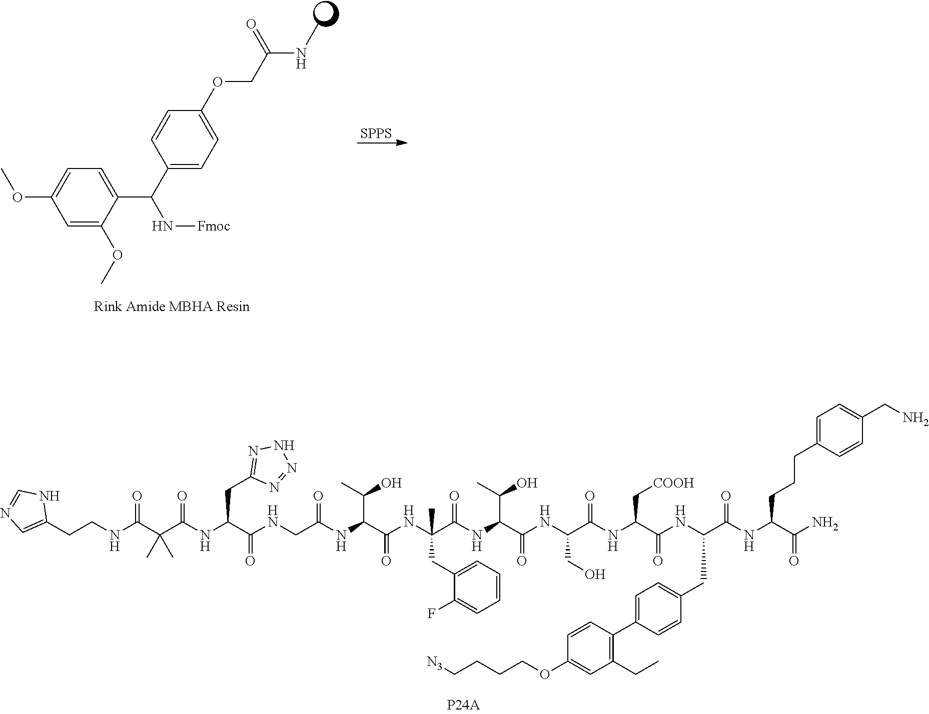

The peptide elongation was performed on a 0.5 mmol scale using Liberty Lite Automated Microwave Peptide Synthesizer. To a polypropylene solid-phase reaction vessel was added Rink Amide MBHA Resin (0.5 mmol, 1 eq.). The resin was washed (swelled) two times as follows: to the reaction vessel was added DMF (10 mL) through the top of the vessel upon which the mixture was agitated for 5 minutes before the solvent was drained through the frit.

The general coupling reaction of each amino acid was carried out after general removal of Fmoc group procedure. A) General removal of Fmoc group procedure: To the reaction vessel containing the resin from the previous step was added piperidine: DMF (1:4 v/v, 5 mL). The mixture was agitated under microwave at 90° C. for 2 min and then the solution was drained through the frit. The resin was washed five times as follows: for each wash, DMF (5 mL) was added through the top of the vessel and the resulting mixture was periodically agitated for 0.5 minutes before the solution was drained through the frit. B) General coupling reaction procedure:

To the reaction vessel was added the amino acid (0.2 M in DMF, 12.5 mL, 5 eq.), then DIC (0.5 M in DMF, 4 mL, 4 eq.) and oxyma (0.5 M in DMF, 2 mL, 2 eq.). The mixture was agitated under microwave at 90° C. for 10 min, then the reaction solution was drained through the frit. The resin was washed four times as follows: for each wash, DMF (8 mL) was added through the top of the vessel and the resulting mixture was periodically agitated for 0.5 minutes before the solution was drained through the frit. After completion of synthesis, resin was thoroughly rinsed with DMF (6×6 mL) then CH₂Cl₂ (6×6 mL). The resulting resin was subjected to acidic cleavage by using TFA cocktail (TFA/TIPS/H₂O=95:2.5:2.5) for 2 hours, then filtered and the filtrate was diluted with t-BuOMe to give a precipitate, which was centrifuged (5000 R) for 10 min and decanted to give a crude product.

The corresponding aa10-aa9-aa8-aa7-aa6-aa5-aa4-aa3-aa2-aa1b peptidyl Rink Amide MBHA Resin was prepared as described in the general procedure of SPPS. The crude product was purified by prep-HPLC (column: Phenomenex Gemini-NX C18 80*30 mm*5 μm; mobile phase: [water (0.1% TFA)-ACN]; B %: 5%-55%, 8 min) to afford pure product P24A (50 mg, 31.79 μmol, 41.80% yield) as a white solid.

LCMS (ESI): RT=2.913 min, m/z calcd. C₇₅H₁₀₁FN₂₀O₁₇ 786.37, found 786.9 [M+2H]²⁺. LCMS conditions: 1.5 ML/4LTFA in water (solvent A) and 0.75 ML/4 L TFA in acetonitrile (solvent B), using the gradient 10%-80% (solvent B) over6 minutes and holding at 80% for 0.5 minutes at a flow rate of 0.8 ml/min; Column: Xtimate 3 μm, C18, 2.1*30 mm.

HPLC: RT=7.44 min, 99.18% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.36 Preparation of (8S,14S,17S,20S,23S,26S)-8-((2H-tetrazol-5-yl)methyl)-26-(((S)-1-(((S)-1-amino-5-(4-(13-amino-3,6,9,12-tetraoxo-2,5,8,11-tetraazatridecyl)phenyl)-1-oxopentan-2-yl)amino)-3-(4′-(4-azidobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P32) (SEQ ID NOS 600 and 495, respectively, in order of appearance)

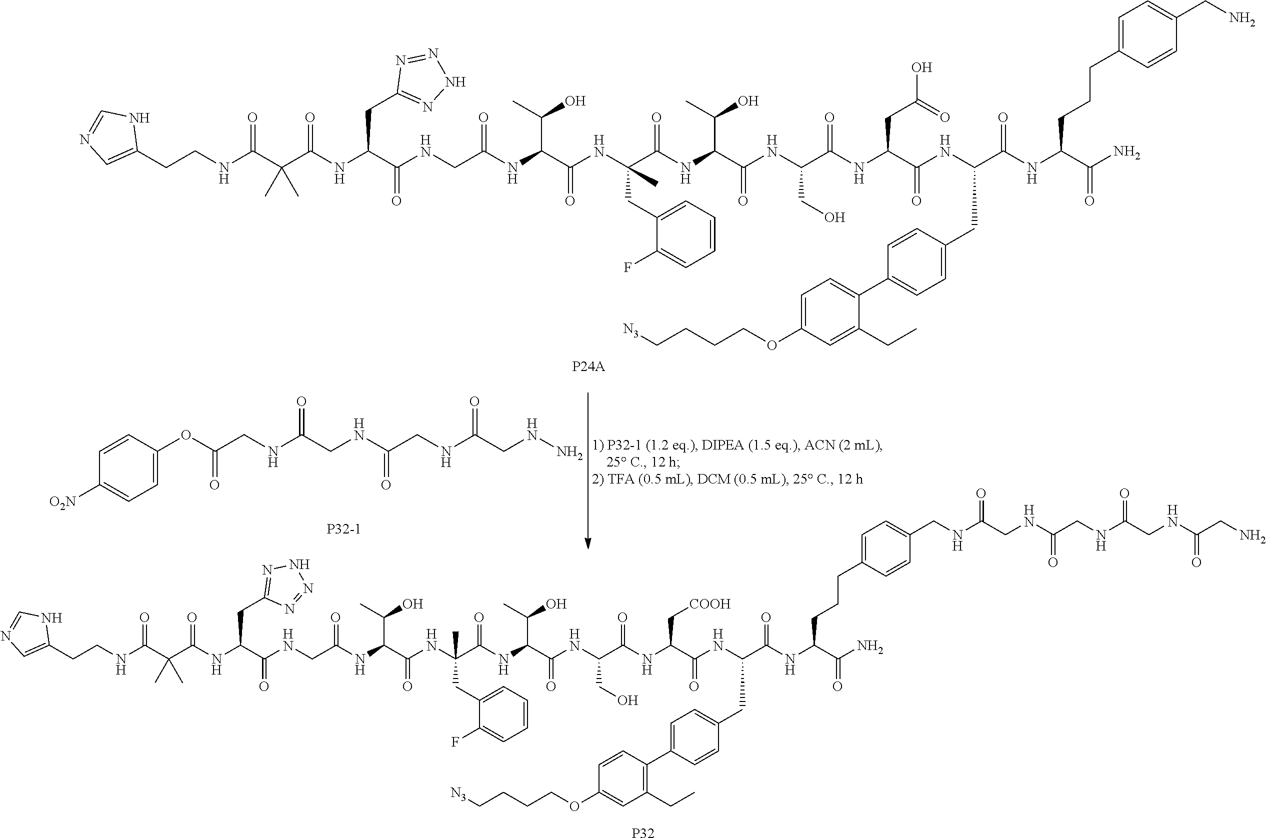

To a solution of P24A (20 mg, 12.72 μmol, 1 eq.) in DMF (2 mL) were added P32-1 (7.13 mg, 15.26 μmol, 1.2 eq.) and DIPEA (3.29 mg, 25.43 μmol, 4.43 μL, 2.0 eq.). Then the solution was stirred at 20° C. for 2 hr. After completion, water (6 mL) was added and the mixture was lyophilized to give a white solid (25 mg, crude), which was added in DCM (2.5 mL), followed by the addition of TFA (3.85 g, 33.77 mmol, 2.5 mL, 2567.52 eq.). Then the solution was stirred at 20° C. for 2 hr. After completion, the solvent of the solution was removed under reduced pressure to give the crude. It was purified by prep-HPLC (column: Phenomenex Gemini-NX 80*40 mm*3 μm; mobile phase: [water (0.1% TFA)-ACN]; B %: 5%-45%, 30 min). P32 (7.2 mg, 3.60 μmol, 27.36% yield, 90% purity) was obtained as a white solid.

LCMS: (ESI): Rt=2.880 min, mass calcd. for C₃₂H₁₁₂FN₂₅O₂₁ 901.20, found 900.91 [M+2H]²⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC: RT=7.23 min, 100% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.37 Preparation of (8S,14S,17S,20S,23S,26S)-8-((2H-tetrazol-5-yl)methyl)-26-(((S)-1-(((S)-1-amino-5-(4-(17-hydroxy-3-oxo-6,9,12,15-tetraoxa-2-azaheptadecyl)phenyl)-1-oxopentan-2-VI)amino)-3-(4′-(4-azidobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P33) (SEQ ID NOS 600 and 496, respectively, in order of appearance)

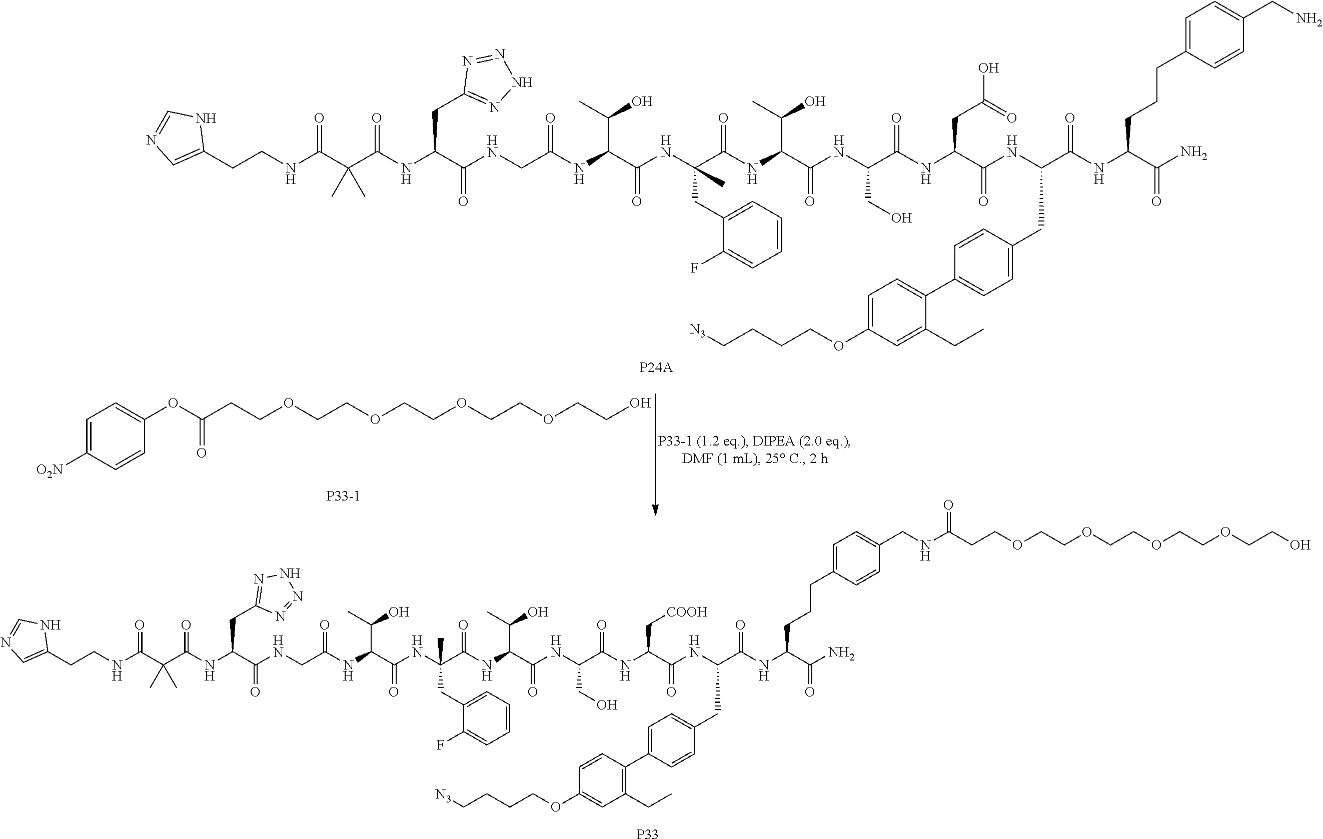

To a solution of P24A (10 mg, 6.36 μmol, 1 eq.) in DMF (0.3 mL) were added P33-1 (2.96 mg, 7.63 μmol, 1.2 eq.) and DIPEA (1.64 mg, 12.72 μmol, 2.22 μL, 2.0 eq). Then the solution was stirred at 20° C. for 2 hr. After completion, the reaction mixture was purified by prep-HPLC (TFA condition, column: Boston Green ODS 150*30 mm*5 μm; mobile phase: [water (0.1% TFA)-ACN]; B %: 14%-54%, 9 min) to afford P33 (2.0 mg, 1.13 μmol, 17.69% yield, 95% purity) as a white solid.

LCMS: (ESI): Rt=3.383 min, mass calcd. for C₈₅H₁₂₀FN₂₁O₂₃ 911.40, found 911.40 [M+2H]²⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC: RT=7.96 min, 95.47% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.38 Preparation of (8S,14S,17S,20S,23S,26S)-8-((2H-tetrazol-5-yl)methyl)-26-(((S)-1-(((S)-1-amino-5-(4-(29,29-dimethyl-3,6,9,12,15,18,21,24,27-nonaoxo-28-oxa-2,5,8,11,14,17,20,23,26-nonaazatriacontyl)phenyl)-1-oxopentan-2-yl)amino)-3-(4′-(4-azidobutoxy)-2′-ethyl-(1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P34) (SEQ ID NOS 495 and 497, respectively, in order of appearance)

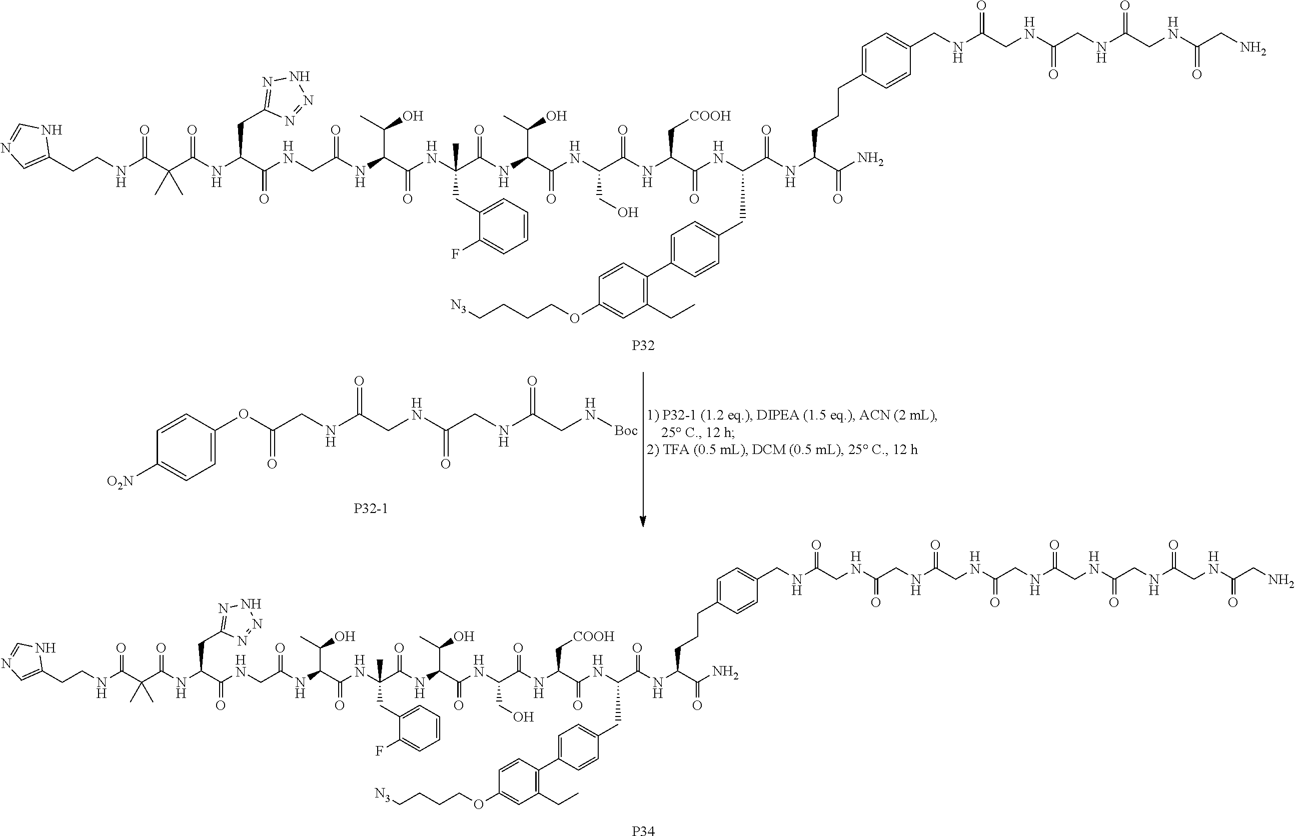

To a solution of P32 (5 mg, 2.78 μmol, 1 eq) in DMF (1 mL) were added P32-1 (1.56 mg, 3.33 μmol, 1.2 eq) and DIPEA (717.64 ug, 5.55 μmol, 9.67e-1 μL, 2.0 eq.). Then the solution was stirred at 20° C. for 2 hr. After completion, water (6 mL) was added and the mixture was lyophilized to give a white solid (25 mg, crude), which was added in DCM (0.2 mL), followed by the addition of TFA (256.67 mg, 2.25 mmol, 166.67 μL, 958.60 eq.). Then the solution was stirred at 20° C. for 2 hr. After completion, the solvent of the solution was removed under reduced pressure to give the crude. It was purified by prep-HPLC (column: Waters Xbridge BEH C18 100*25 mm*5 μm; mobile phase: [water (0.1% TFA)-ACN]; B %: 10%). P34 (1.72 mg, 7.63e-1 μmol, 32.49% yield, 90% purity) was obtained as a white solid.

LCMS: (ESI): Rt=2.760 min, mass calcd. for C₉₀H₁₂₄FN₂₉O₂₅ 1014.98, found 1015.20 [M+2H]²⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC: RT=7.15 min, 100% purity. HPLC method A: Column: YMC-Pack ODS-A 150*4.6 mm, 5 μm; 2.75 ML/4 LTFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### 3.39 Preparation of (8S,14S,17S,20S,23S,26S)-8-((2H-tetrazol-5-yl)methyl)-26-(((S)-1-(((S)-1-amino-5-(4-(17-hydroxy-3-oxo-6,9,12,15-tetraoxa-2-azaheptadecyl)phenyl)-1-oxopentan-2-yl)amino)-3-(4′-(4-azidobutoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (P35) (SEQ ID NOS 600 and 498, respectively, in order of appearance)

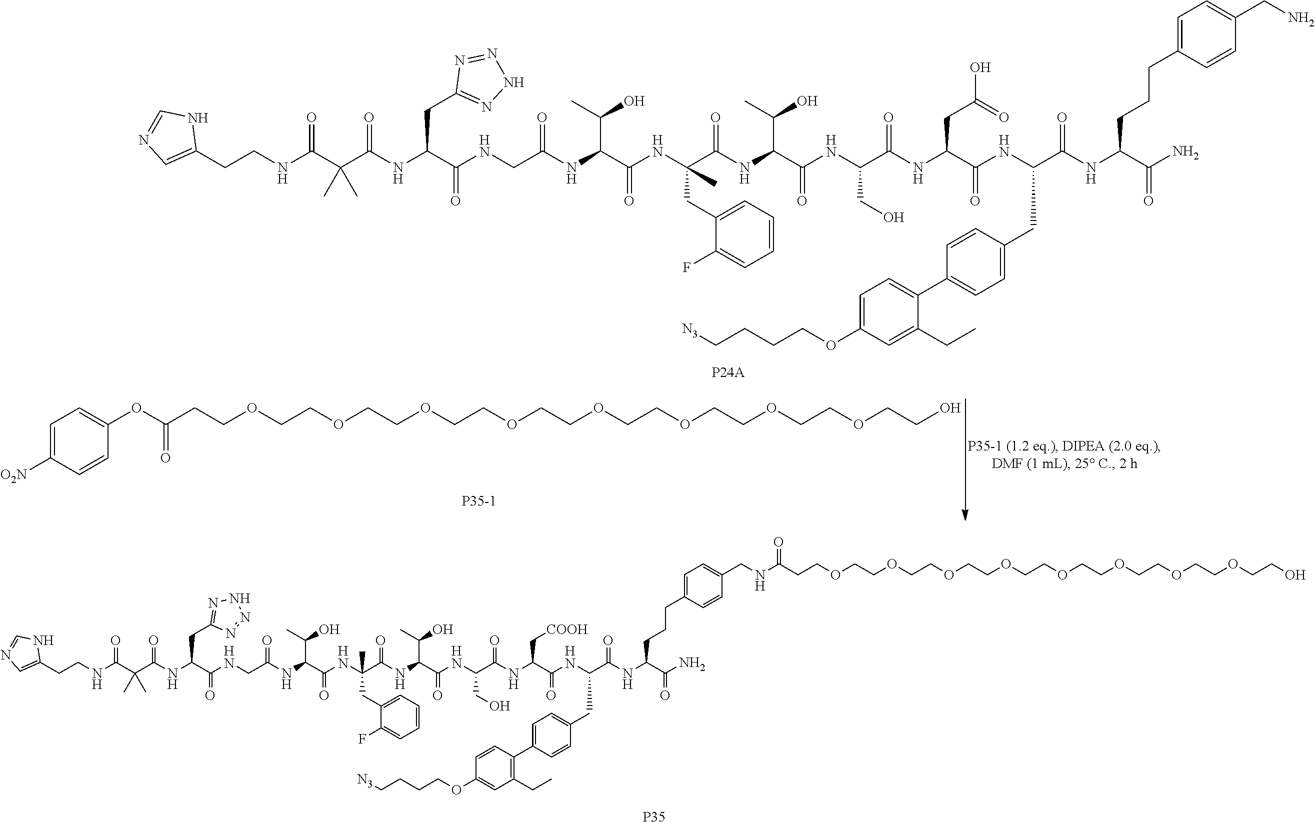

To a solution of P24A (20 mg, 12.72 μmol, 1 eq.) in DMF (0.3 mL) were added P35-1 (10.75 mg, 19.08 μmol, 1.5 eq.) and DIPEA (3.29 mg, 25.43 μmol, 4.43 μL, 2.0 eq.). Then the solution was stirred at 20° C. for 2 hr. After completion, the reaction mixture was purified by prep-HPLC (column: Boston Green ODS 150*30 mm*5 μm; mobile phase: [water (0.1% TFA)-ACN]; B %: 14%-54%, 9 min) to afford P33 (20 mg, 10.01 μmol, 78.75% yield, 100% purity) as a white solid.

LCMS: (ESI): Rt=3.325 min, mass calcd. for C₉₃H₁₃₆FN₂₁O₂₇ 998.98, found 999.40 [M+2H]²⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC: RT=7.88 min, 100% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

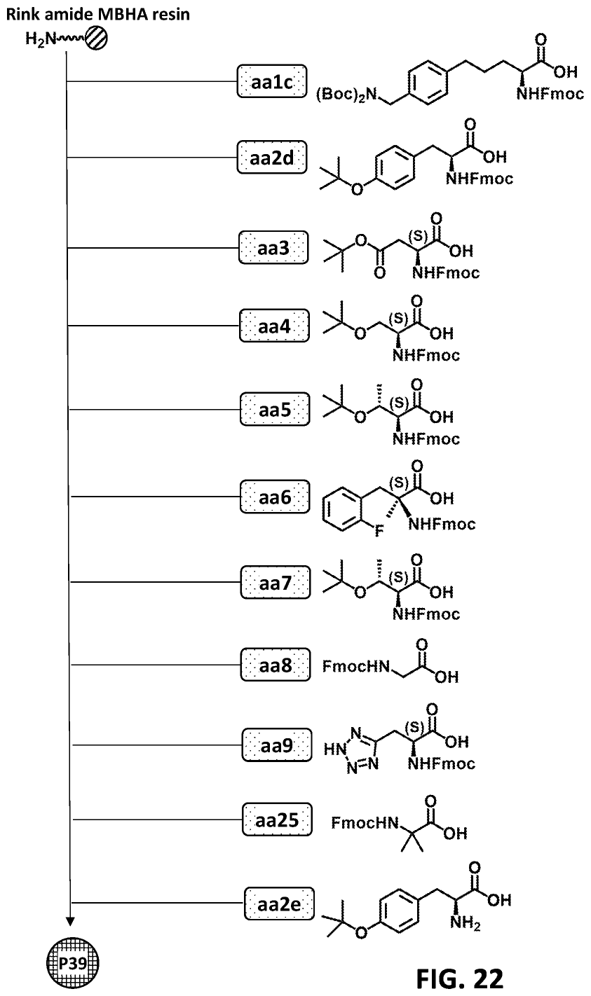

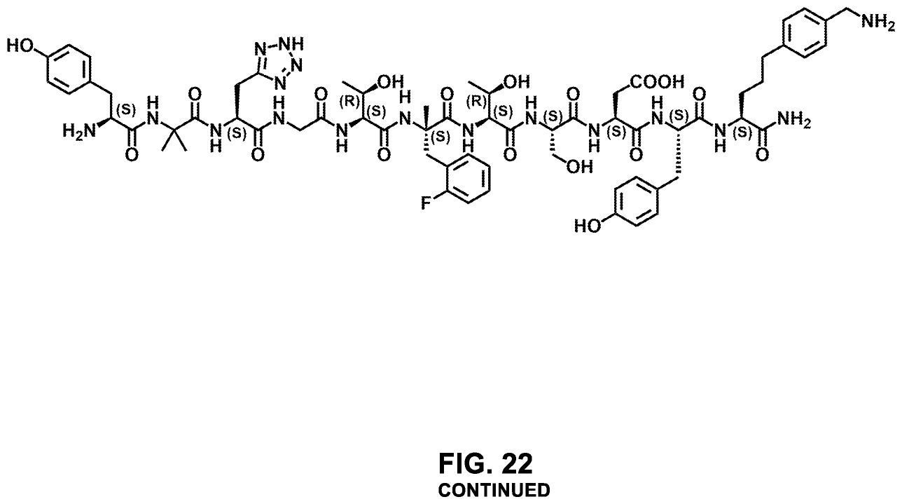

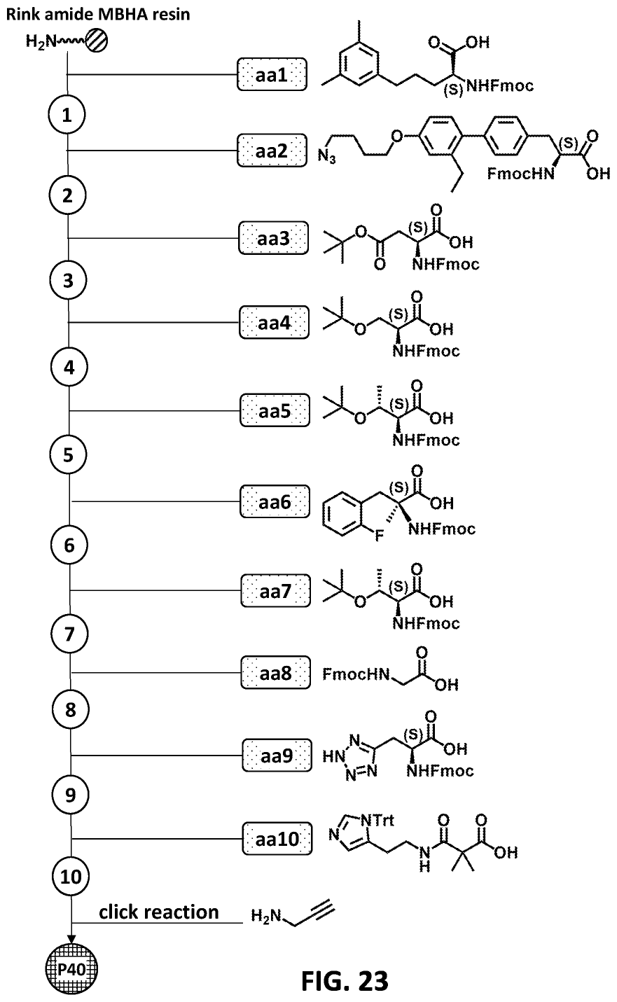

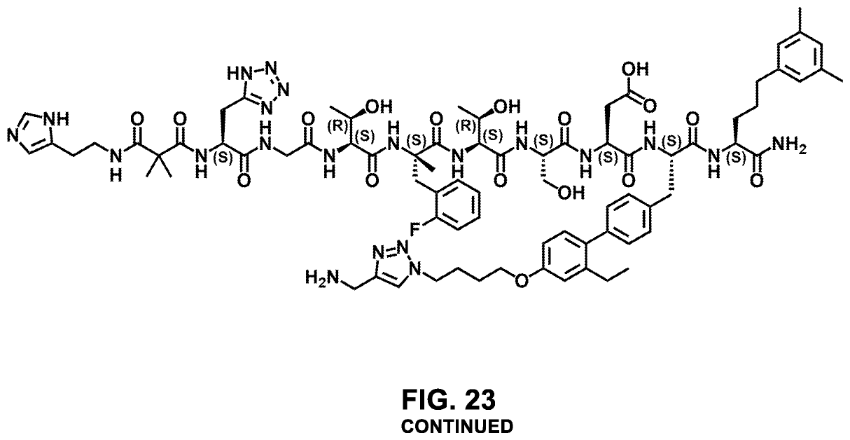

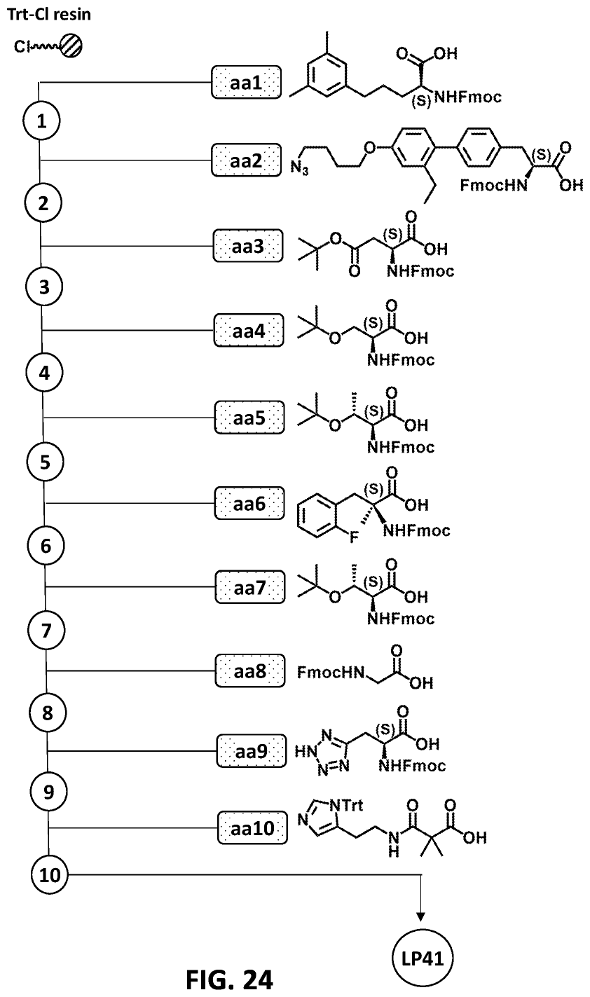

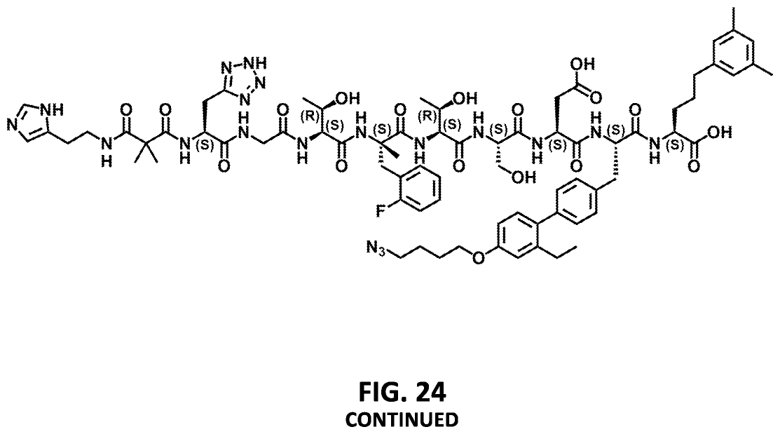

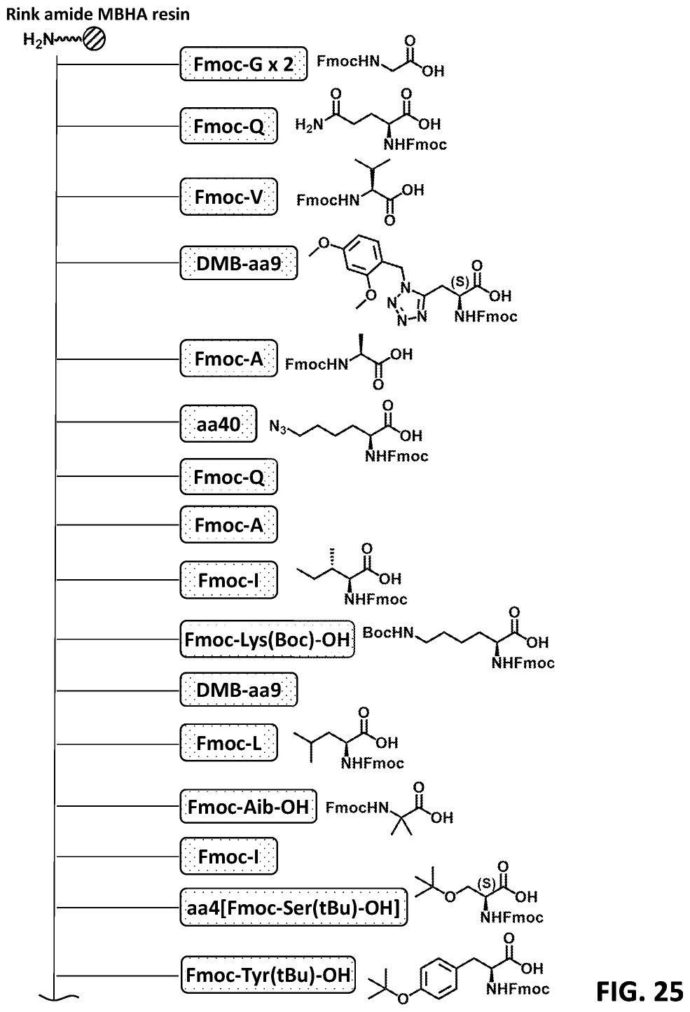

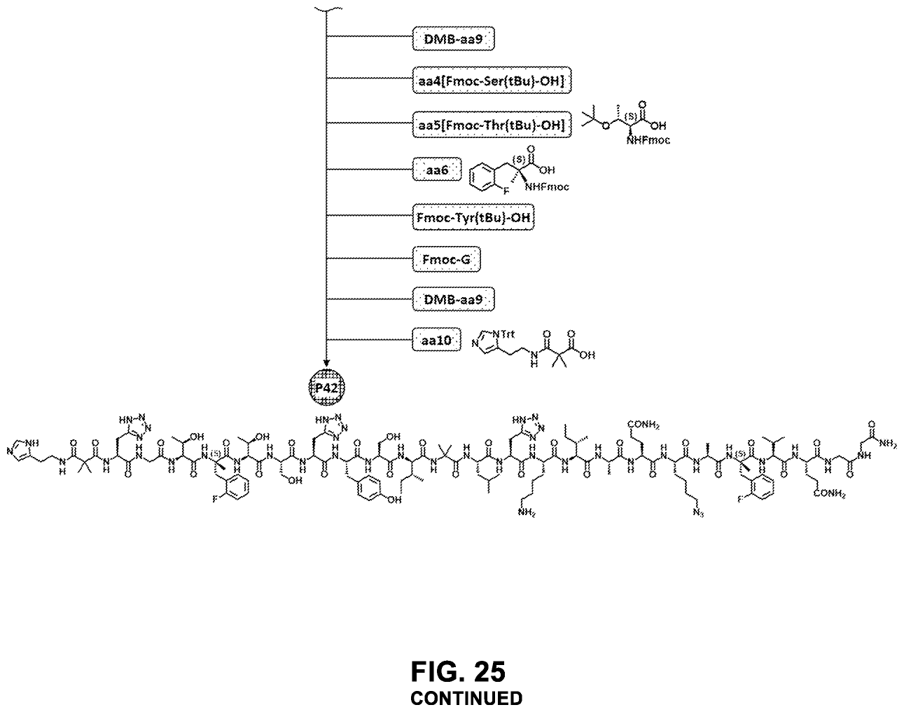

FIG. 22 depicts the sequence for synthesis of P39.

### Preparation of (3S,6S,9S,12S,15S,21S,27S)-21-((2H-tetrazol-5-yl)methyl)-27-amino-3-(((S)-1-(((S)-1-amino-5-(4-(aminomethyl)phenyl)-1-oxopentan-2-yl)amino)-3-(4-hydroxyphenyl)-1-oxopropan-2-yl)carbamoyl)-12-(2-fluorobenzyl)-9,15-bis((R)-1-hydroxyethyl)-6-(hydroxymethyl)-28-(4-hydroxyphenyl)-12,24,24-trimethyl-5,8,11,14,17 20,23,26-octaoxo-4,7,10,13,16,19,22,25-octaazaoctacosan-1-oic acid (P39) (SEQ ID NO: 502)

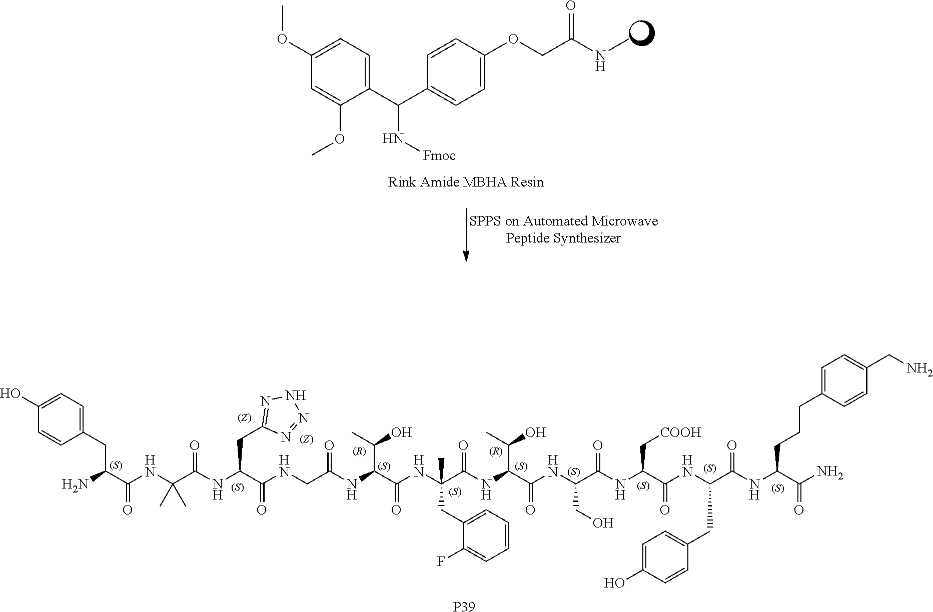

The peptide elongation was performed on a 0.5 mmol scale using Liberty Lite Automated Microwave Peptide Synthesizer. Following the standard operation on peptide synthesizer: a) De-protection: a solution of 20% piperidine/DMF (5 mL) was added to the resin vessel, agitated with N₂ for 2 min at 90° C. Then drained the vessel and washed with DMF (3 mL×3) at 20° C. b) Coupling (each amino acid reacted for triple with 5.0 eq.): a solution of amino acid (2.5 mmol, 5 eq.) in DMF (5 mL), DIC (2 mL) and oxyma (1 mL) were added to the vessel and agitated with N₂ for 10 min at 90° C. Repeat a) and b) for all amino acids. The resin was subjected to acidic cleavage by using TFA cocktail (TFA/TIPS/H₂O=95:2.5:2.5), then filtered and the filtrate was diluted with t-BuOMe to give a precipitate, which was centrifuged (5000 R) for 10 min to give the crude product.

The corresponding aa24-aa25-aa9-aa8-aa7-aa6-aa5-aa4-aa3-Y-aa1b peptidyl Rink Amide MBHA Resin was prepared as described in the general procedure of SPPS. The crude product was purified by prep-HPLC (column: Boston Prime C18 150*30 mm*5 μm; mobile phase: [water (0.05% ammonia hydroxide v/v)-ACN]; B %: 0%-35%, 9 min) to afford pure product P39 (15 mg, 9.35 μmol, 9.35% yield, 88% purity) as a white solid. The product was further purified by prep-HPLC (column: Welch Xtimate C18 100*40 mm*3 μm; mobile phase: [water (0.075% TFA)-ACN]; B %: 5%-45%, 12 min) to give the product (10 mg, 6.96 μmol, 65.51% yield, 98.26% purity) was obtained as a white solid.

LCMS (ESI): RT=1.573 min, m/z calcd. for C65H87FN17018 [M+H]⁺ 1412.63, C65H88FN17018 [M+2H]²+707.81, found C₆₅H₈₇FN₁₇O₁₈ [M+H]⁺1412.70, C₆₅H₈₈FN₁₇O₁₈ [M+2H]²+707.30 found. LCMS conditions: Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC: RT=4.72 min, 98.26% purity LC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### Example 4. Synthesis of Linkers

### 4.1 Preparation of PEG Linkers

Scheme 20, below, depicts synthesis of PEGn linkers (n=4, 8, 12 and 24):

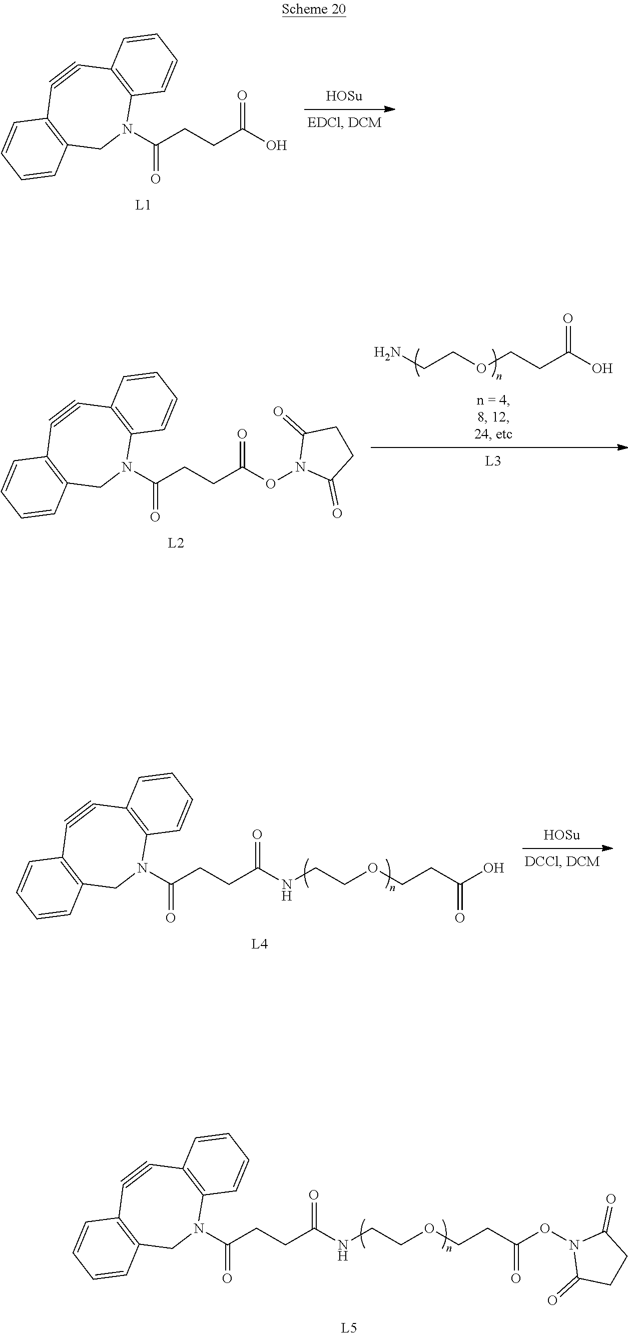

Synthesis of PEGn linker with n=8 is provided as an example; linkers of other lengths were prepared in the same fashion. Synthesis of L1 ((11,12-Didehydrodibenzo[b,f]azocin-5(6H)-yl)-4-oxobutanoic acid) was performed according to L. S. Campbell-Verduyn, L. Mirfeizi, A. K. Schoonen, R. A. Dierckx, P. H. Elsinga, and B. L. Feringa, Strain-Promoted Copper-Free “Click” Chemistry for ¹⁸F Radiolabeling of Bombesin. Angew. Chem. Int. Ed., Vol. 50, No. 47, 2011, 11117-11120.

### Step 1: Synthesis of 2,5-dioxopyrrolidin-1-yl 4-(didehydrodibenzo[b,f]azocin-5(6H)-yl)-4-oxobutanoate (L2)

To a solution of L1 (0.35 g, 1.15 mmol, 1 eq.) in DCM (4 mL) were added HOSu (158.31 mg, 1.38 mmol, 1.2 eq.) and EDCl (263.70 mg, 1.38 mmol, 1.2 eq.). The mixture was stirred at 20° C. for 1 hr. TLC (PE:EtOAc=1:1) indicated that the complete consumption of reactant. The mixture was filtered and concentrated to give a residue. The residue was purified by column chromatography (SiO₂, Petroleum ether/Ethyl acetate=3/1 to 1/1). The desired compound L2 (450 mg, 1.12 mmol, 97.56% yield) was obtained as a white solid.

### Step 2: Synthesis of 3-[2-[2-[2-[2-[2-[2-[2-[2-[[4-(39-azatricyclohexadeca-(4), 1(5),2(6),3(7),31,33-hexaen-9-yn-39-yl)-4-oxo-butanoyl] amino] ethoxy] ethoxy] ethoxy] ethoxy] ethoxy] ethoxy] ethoxy]ethoxy] propanoic acid (L4)

To a solution of L2 (54.68 mg, 135.90 μmol, 1.5 eq.) in DMF (0.5 mL) was added L3 (n=8, 40 mg, 90.60 μmol, 1 eq.) and DIPEA (58.54 mg, 452.99 μmol, 78.90 μL, 5 eq.). The mixture was stirred at 25° C. for 1 hr. LCMS trace indicated that the complete consumption of reactant and the formation of the desired mass. The mixture was filtered and purified by prep-HPLC (AcOH 0.3%, MeCN/H₂O, 0˜43%, 25 mL/min, 15 min). The desired L4 (60 mg, 74.09 μmol, 81.78% yield, 90% purity) was obtained as a pale-yellow oil.

LCMS (ESI): RT=0.900 min, mass calcd. for C₃₃H₅₃N₂O₁₂ 729.36, m/z found 729.3 [M+H]⁺. Reverse phase LC-MS was carried out using a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

### Step 3: Synthesis of (2,5-dioxopyrrolidin-1-yl) 3-[2-[2-[2-[2-[2-[2-[2-[2-[[4-(43-azatricyclohexadeca-(4),1(5),2(6),3(7),33,35-hexaen-11-yn-43-yl)-4-oxo-butanoyl] amino] ethoxy] ethoxy] ethoxy]ethoxy] ethoxy]ethoxy]ethoxy]ethoxy]propanoate (L5)

To a solution of L4 (20 mg, 27.44 μmol, 1 eq.) in DCM (0.5 mL) was added HOSu (6.32 mg, 54.88 μmol, 2 eq.) and DCC (8.49 mg, 41.16 μmol, 8.33 μL, 1.5 eq.). The mixture was stirred at 25° C. for 1 hr. LCMS trace indicated that the complete consumption of reactant and the formation of the desired mass. The mixture was filtered and purified by prep-HPLC (AcOH 0.3%, MeCN/H₂O, 0˜65%, 18 mL/min, 15 min). The desired compound L5 (18 mg, 17.87 μmol, 65.13% yield, 82% purity) was obtained as a pale-yellow oil.

LCMS (ESI): RT=0.920 min, mass calcd. for C₄₂H₅₅N₃O₁₄ 825.37, m/z found 848.2 [M+Na]⁺. Reverse phase LC-MS was carried out using a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

### 4.2 Preparation of Peptide Linker L8

Scheme 21, below, depicts synthesis of SG4-SH peptide linker L8:

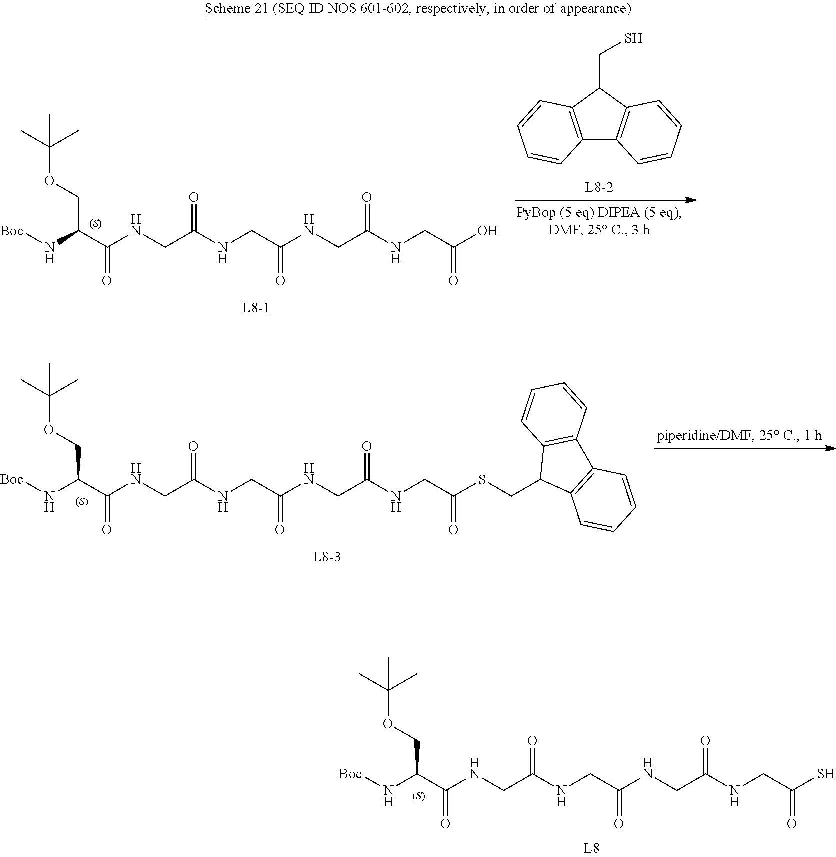

See the following references D. Crich, K. Sana, and S. Guo, Amino Acid and Peptide Synthesis and Functionalization by the Reaction of Thioacids with 2,4-Dinitrobenzenesulfonamides. Org. Lett., Vol. 9, No. 22, 2007, 4423-4426 and X. Y. Wu, J. L. Stockdill, P. K. Park, J. Samuel, and S. J. Danishefsky, Expanding the Limits of Isonitrile-Mediated Amidations: On the Remarkable Stereosubtleties of Macrolactam Formation from Synthetic Seco-Cyclosporins. J. Am. Chem. Soc., Vol. 134, No. 4, 2012, 2378-2384.

### Step 1: (S)—S-((9H-fluoren-9-yl)methyl) 6-(tert-butoxymethyl)-2,2-dimethyl-4,7,10,13,16-pentaoxo-3-oxa-5,8,11,14,17-pentaazanonadecane-19-thioate (L8-3)

To a solution of L8-1 (700 mg, 1.43 mmol, 1 eq.) in DMF (7 mL) were added 4A MOLECULAR SIEVE (1 g) and L8-2 (455.40 mg, 2.14 mmol, 1.5 eq.) and the mixture was stirred at −20° C. After 15 min, PyBOP (1.12 g, 2.14 mmol, 1.5 eq.) and DIPEA (369.63 mg, 2.86 mmol, 498.15 μL, 2 eq.) were added and the reaction mixture was stirred at −20° C. for 1.5 h. LCMS trace showed that the reaction converted completely. The reaction was diluted with EtOAc (30 mL) and filtered, the cake was washed with EtOAc (10 mL*2). The filtrate was washed with aq.NH₄Cl (20 mL), water (20 mL), brine (20 mL), dried over anhydrous Na₂SO₄, filtered and concentrated in vacuum to give crude as yellow oil. The residue was purified by flash silica gel chromatography (ISCO@; 40 g SepaFlash@ Silica Flash Column, Eluent of 0˜20% MeOH/DCM gradient @ 30 mL/min) to give L8-3 (700 mg, 814.78 μmol, 56.98% yield, 79.594% purity) as a light-yellow oil.

LCMS: (ESI): RT=0.882 min, m/z calcd. for C₂₉H₃₈N₅O₆S, 584.25 [M−Boc+2H]²⁺, m/z found 584.3 [M−Boc+2H]²⁺; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

¹H NMR (400 MHz, METHANOL-d4) 6=7.78 (d, J=7.3 Hz, 2H), 7.66 (d, J=7.2 Hz, 2H), 7.42-7.29 (m, 4H), 4.15 (d, J=5.0 Hz, 2H), 4.04 (s, 2H), 3.92 (d, J=4.3 Hz, 6H), 3.68-3.57 (m, 4H), 3.38-3.35 (m, 3H), 1.46 (s, 9H), 1.19 (s, 1OH).

### Step 2: (S)-6-(tert-butoxymethyl)-2,2-dimethyl-4,7,10,13,16-pentaoxo-3-oxa-5,8,11,14,17-pentaazanonadecane-19-thioic S-acid (L8)

To a solution of L8-3 (560 mg, 818.94 μmol, 1 eq.) in THF (7 mL) was added piperidine (139.46 mg, 1.64 mmol, 161.75 μL, 2 eq.) at 20° C. The reaction was stirred at 20° C. for 2 h. LCMS trace showed that the reaction converted completely. The reaction was added MTBE (50 mL), white solids were precipitated. The mixture was filtered to give a crude as an off-white solid. The crude was purified by prep-HPLC (reversed-phase column, 40 g, 0%-35% 0.4% AcOH in water/ACN, 15 min) to give L8 (180 mg, 252.88 μmol, 30.88% yield, 71.028% purity) as an off-white solid.

LCMS: (ESI): RT=0.709 min, m/z calcd. for C₁₅H₂₈N₅O₆S, 406.18 [M−Boc+2H]⁺, m/z found 406.2 [M−Boc+2H]⁺; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

### 4.3 Preparation of Peptide Linker L9

Scheme 22, below, depicts synthesis of SG4-SH peptide linker L8:

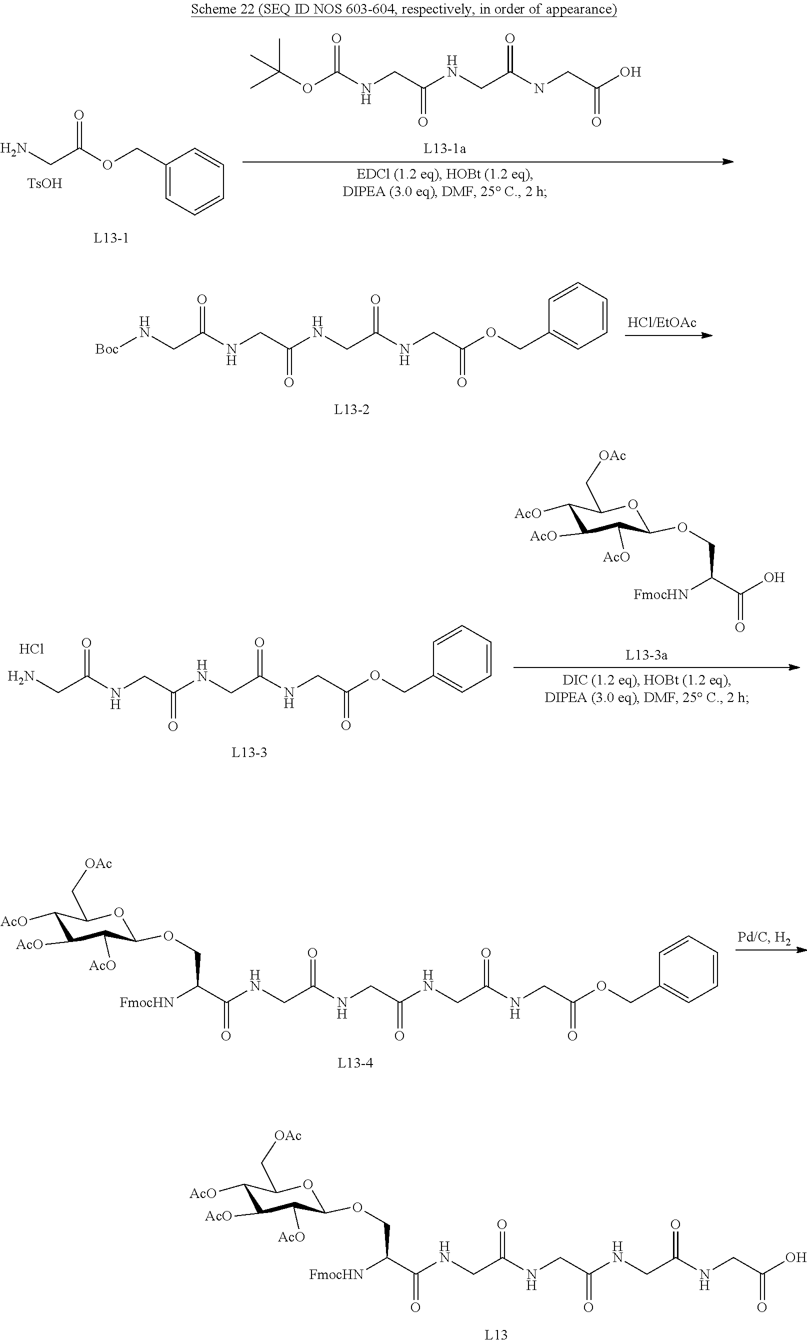

Glycopeptide L13-3a, was synthesized according to J. J. Du, X. F. Gao, L. M. Xin, Z. Lei, Z. Liu, and J. Guo, Convergent Synthesis of N-Linked Glycopeptides via Aminolysis of ω-Asp p-Nitrophenyl Thioesters in Solution. Org. Lett., Vol. 18, No. 19, 2016, 4828-4831. Synthesis of L13-1awas performed according to Y. A. Naumovich, I. S. Golovanov, A. Y. Sukhorukov, and S. L. Loffe, Addition of HO-Acids to N,N-Bis(oxy)enamines: Mechanism, Scope and Application to the Synthesis of Pharmaceuticals. Eur. J. Org. Chem., Vol. 2017, No. 4, 2017, 6209-6227.

### Step 1: Synthesis of benzyl 2,2-dimethyl-4,7,10,13-tetraoxo-3-oxa-5,8,11,14-tetraazahexadecan-16-oate (L13-2)

To a solution of L13-1a (10 g, 34.57 mmol, 1 eq.) in DMF (60 mL) was added HOBt (5.61 g, 41.48 mmol, 1.2 eq.), DIPEA (22.34 g, 172.84 mmol, 30.10 mL, 5 eq.) and EDCl (7.95 g, 41.48 mmol, 1.2 eq.) at 20° C. The mixture was stirred at 20° C. for 15 min, L13-1 (11.08 g, 32.84 mmol, 0.95 eq.) was added and the reaction mixture was stirred at 20° C. for 2 h. The reaction progress was monitored by LC-MS, which indicated no starting material was remained and formation of desired product. The mixture was quenched with a saturated solution of NaHCO₃ (20 mL) and brine (20 mL×3), the solid precipitation was collected and washed with PE (20 mL×2), concentrated under reduced pressure to give L13-2 (13.5 g, 29.38 mmol, 85.00% yield, 95% purity) as a white solid.

LCMS: (ESI): RT=0.714 min, m/z calcd. for C₂₀H₂₃N₄O₇Na 459.2 [M+Na]⁺, found 459.1; LC-MS Conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

¹H NMR (400 MHz, DMSO-d6) δ=8.31 (br t, J=5.7 Hz, 1H), 8.19 (br t, J=5.8 Hz, 1H), 8.05 (br t, J=5.3 Hz, 1H), 7.43-7.29 (m, 5H), 7.04-6.95 (m, 1H), 5.13 (s, 2H), 3.90 (d, J=5.9 Hz, 2H), 3.75 (d, J=5.6 Hz, 4H), 3.58 (br d, J=6.0 Hz, 2H), 1.38 (s, 9H).

### Step 2: Synthesis of benzyl 2-(2-(2-(2-aminoacetamido)acetamido)acetamido)acetate hydrochloride (L13-3)

A solution of HCl/EtOAc (4 M, 14.80 mL, 4 eq.) was added to L13-2 (7 g, 14.80 mmol, 1 eq, HCl) dropwise at 20° C. The mixture was stirred at 20° C. for 10 min. The reaction progress was monitored by LC-MS, which indicated no starting material was remained and formation of desired product. The mixture was concentrated in vacuum and lyophilization to provide the L13-3 (5.5 g, 10.33 mmol, 69.77% yield, 70% purity, HCl) as a white solid.

LCMS: (ESI): RT=0.479 min, m/z calcd. for C₁₅H₂₁N₄O₅ 337.1 [M+H]⁺, found 337.1; LC-MS Conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

### Step 3: Synthesis of (2R,3R,4S,5R,6R)-2-(((S)-16-((((9H-fluoren-9-yl)methoxy)carbonyl)amino)-3,6,9,12,15-pentaoxo-1-phenyl-2-oxa-5,8,11,14-tetraazaheptadecan-17-yl)oxy)-6-(acetoxymethyl)tetrahydro-2H-pyran-3,4,5-triyl triacetate (L13-4)

To a solution of L13-3a (2.9 g, 4.41 mmol, 1 eq.) in DMF (20 mL) was added HOBt (715.03 mg, 5.29 mmol, 1.2 eq.), DIPEA (3.42 g, 26.46 mmol, 4.61 mL, 6 eq.) and DIC (667.82 mg, 5.29 mmol, 819.42 μL, 1.2 eq.) at 20° C. The mixture was stirred at 20° C. for 15 min, L13-3 (3.29 g, 6.61 mmol, 1.5 eq, HCl) was added and the reaction mixture was stirred at 20° C. for 12 h. The reaction progress was monitored by LC-MS, which indicated no starting material was remained and formation of desired product. The mixture was quenched with water (40 mL), and extracted with ethyl acetate (40 mL×2). The organic layer was washed with water and brine (30 mL×3), dried over anhydrous Na₂SO₄, filtered, and concentrated in vacuum. The residue was purified by flash silica gel chromatography (ISCO@; 40 g SepaFlash@ Silica Flash Column, Eluent of 0˜5% MeOH/DCM) for 25 min with total volume 1.6 L to provide L13-4 (2.1 g, 1.72 mmol, 39.04% yield, 80% purity) as a white solid.

LCMS: (ESI): RT=1.937 min, m/z calcd. for C₄₇H₅₄N₅O₁₈ 976.3 [M+H]⁺, found 976.3; LC-MS Conditions: Mobile Phase: 1.5 ML/4 L TFA in water (solvent A) and 0.75 ML/4 L TFA in acetonitrile (solvent B),using the elution gradient 10%-80% (solvent B) over 2.0 minutes and holding at 80% for 0.48 minutes at a flow rate of 0.8 ml/min; Column: Xtimate 3 μm, C18,2.1*30 mm.

¹H NMR (400 MHz, CHLOROFORM-d) δ=7.83-7.73 (m, 2H), 7.59 (br d, J=7.5 Hz, 2H), 7.47-7.28 (m, 10H), 7.21-7.03 (m, 2H), 5.87 (br d, J=5.5 Hz, 1H), 5.27-4.89 (m, 5H), 4.61-4.38 (m, 4H), 4.29-3.81 (m, 13H), 2.10-1.98 (m, 12H).

### Step 4: Synthesis of (S)-1-(9H-fluoren-9-yl)-3,6,9,12,15-pentaoxo-5-((((2R,3R,4S,5R,6R)-3,4,5-triacetoxy-6-(acetoxymethyl)tetrahydro-2H-pyran-2-yl)oxy)methyl)-2-oxa-4,7,10,13,16-pentaazaoctadecan-18-oic acid (L13)

To a solution of L13-4 (2.1 g, 2.15 mmol, 1 eq.) in EtOAc (16 mL) and MeOH (2 mL) was added Pd/C (300 mg, 430.35 μmol, 10.35 μL, 10% purity, 0.20 eq.) at 20° C. and the mixture was stirred at 20° C. for 4 hr under H₂ (4.35 mg, 2.15 mmol, 1 eq.) (15 psi). The reaction progress was monitored by LC-MS, which indicated no starting material was remained and formation of desired product. The mixture was filtered and the filtered cake was washed with MeOH (10 mL×3), concentrated in vacuum to provide L13 (1.2 g, 1.29 mmol, 59.81% yield, 95% purity) as a white solid.

LCMS: (ESI): RT=0.788 min, m/z calcd. for C₄₀H₄₈N₅O₁₃ 886.3 [M+H]⁺, found 886.3; LC-MS Conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

¹H NMR (400 MHz, METHANOL-d4) δ=7.81 (br d, J=7.4 Hz, 2H), 7.73-7.63 (m, 2H), 7.44-7.37 (m, 2H), 7.36-7.29 (m, 2H), 5.31-5.18 (m, 1H), 5.03 (t, J=9.8 Hz, 1H), 4.94-4.89 (m, 2H), 4.51-4.20 (m, 5H), 4.16-4.08 (m, 1H), 4.06-3.75 (m, 11H), 2.05-1.98 (m, 12H).

### Example 5. Synthesis of GLP1 Peptidomimetic Linker-Payloads

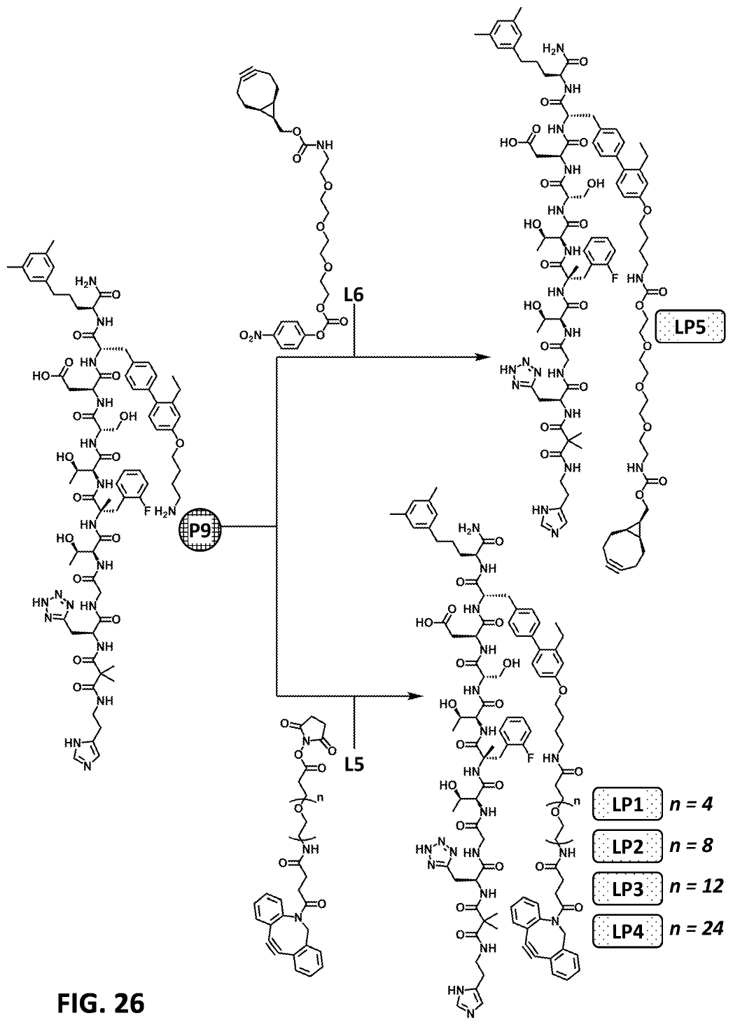

FIG. 26 depicts synthesis of linker-payloads LP1, LP2, LP3, LP4, and LP5 according to the disclosure.

### 5.1 Preparation of (3S)-4-[[(1S)-1-[[4-[4-[4-[3-[2-[2-[2-[2-[[4-(124-azatricyclohexadeca-8(14), 9(15), 10(16),12(18),68,70(73)-hexaen-33-yn-124-yl)-4-oxo-butanoyl]amino] ethoxy] ethoxy] ethoxy] ethoxy]propanoylamino]butoxy]-2-ethyl-72-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (LP1)

To a solution of P9 (9.46 mg, 14.56 μmol, 1.5 eq.) in DMF (0.5 mL) was added L5 (n=4, 15 mg, 9.70 μmol, 1 eq.) and DIPEA (6.27 mg, 48.52 μmol, 8.45 μL, 5 eq.). The mixture was stirred at 25° C. for 1 hr. LCMS trace indicated that the complete consumption of reactant and the formation of the desired mass. The mixture was filtered and purified by prep-HPLC (TFA condition; column: Phenomenex Gemini-NX 150*30 mm*5 μm; mobile phase: [water (10 mM NH₄HCO₃)-ACN]; B %: 30%-60%, 11 min) to give LP1 (4.84 mg, 2.23 μmol, 23.02% yield, 96% purity) as a white solid.

LCMS (ESI): RT=0.944 min, mass calcd. for C₁₀₅H₁₃₅FN₂₀O₂₄ 2078.99, m/z found 1041.0 [M+2H]²⁺. Reverse phase LC-MS was carried out using a Merck RP-18e 25-2 mm column, with a flow rate of 1.5 mL/min, eluting with a gradient of 5% to 95% acetonitrile containing 0.04% TFA (solvent B) and water containing 0.06% TFA (solvent A).

### 5.2 Preparation of 3S)-4-[[(1S)-1-[[4-[4-[4-[3-[2-[2-[2-[2-[2-[2-[2-[2-[[4-(132-azatricyclohexadeca-8(14),9(15),10(16),12(18),76,78(81)-hexaen-33-yn-132-yl)-4-oxo-butanoyl]amino]ethoxy] ethoxy] ethoxy] ethoxy] ethoxy] ethoxy] ethoxy] ethoxy] propanoylamino]butoxy]-2-ethyl-80-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP2)

Starting from P9 (18 mg, 21.79 μmol, 2.25 eq.) and using the same procedure as described in Example 1, the desired LP2 (3.41 mg, 1.41 μmol, 14.63% yield, 94% purity) was obtained as a white solid. Water solubility of LP2 was assessed and determined to be equal to or greater than 60 nM.

LCMS (ESI): RT=0.949 min, mass calcd. for C₁₁₃H₁₅₁FN₂₀O₂₈ 2255.10, m/z found 1128.9 [M+2H]²⁺. Reverse phase LC-MS was carried out using a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=15.273 min. Reverse phase HPLC was carried out using a Gemini-NX 5 μm 150*4.6 mm, C18, 110A column, with a flow rate of 1.0 mL/min, eluting with a gradient of 10% to 80% acetonitrile containing 0.12% TFA (solvent B) and water containing 0.12% TFA (solvent A).

### 5.3 Preparation of (3S)-4-[[(1S)-1-[[4-[4-[4-[3-[2-[2-[2-[2-[2-[2-[2-[2-[2-[2-[2-[2-[[4-(140-azatricyclohexadeca-8(14),9(15),10(16),12(18),84,86(89)-hexaen-33-yn-140-yl)-4-oxo-butanoyl]amino]ethoxy]ethoxy]ethoxy]ethoxy]ethoxy]ethoxy]ethoxy]ethoxy]ethoxy]ethoxy]ethoxy]et hoxy]propanoylamino]butoxy]-2-ethyl-88-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP3)

Starting from P9 (14.59 mg, 14.56 μmol, 1.5 eq.) and using the same procedure as described in Example 1, the desired product LP3 (4.68 mg, 1.68 μmol, 17.27% yield, 87.1% purity) was obtained as a white solid.

HPLC condition: RT=15.120 min, Reverse phase HPLC was carried out using a Gemini-NX 5u C18 110A 150*4.6 mm column, with a flow rate of 1.0 mL/min, eluting with a gradient of 10% to 80% acetonitrile containing 0.1% TFA (solvent B) and water containing 0.1% TFA (solvent A).

### 5.4 Preparation of (3S)-4-[[(1S)-1-[[4-[4-[4-[3-[2-[2-[2-[2-[2-[2-[2-[2-[2-[2-[2-[2-[2-[2-[2-[2-[2-[2-[2-[2-[2-[2-[2-[2-[[4-(164-azatricyclohexadeca-8,10(16),12(18),14(108),15(109),110(113)-hexaen-33-yn-164-yl)-4-oxo-butanoyl]amino]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxyl ethoxy]ethoxy]ethoxy]ethoxy]ethoxy]propanoylamino]butoxy]-2-ethyl-112-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP4)

Starting from P9 (20 mg, 12.94 μmol, 1.0 eq.) and using the same procedure as described in Example 1, the desired product LP4 (5.58 mg, 1.76 μmol, 13.59% yield, 93.29% purity) was obtained as a white solid.

LCMS (ESI): RT=0.912 min, mass calcd. for C₁₄₅H₂₁₅FN₂₀O₄₄ 2961.36 [M+H]⁺, 987.514 [M+3H]³⁺, m/z found 988.3 [M+3H]³⁺. Reverse phase LC-MS was carried out using a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HRMS (ESI): mass calcd for C₁₄₅H₂₁₆FN₂₀O₄₄ 2960.5263 [M+H]⁺, 1480.7632 [M+2H]²+, 987.5088 [M+3H]³⁺, m/z found 2960.53 [M+H]⁺, 1481.26 [M+2H]²+, 987.8517 [M+3H]³+.

### 5.5 Preparation of 8S,14S,17S,20S,23S,26S)-8-((2H-tetrazol-5-yl)methyl)-26-(((S)-1-(((S)-1-amino-5-(3,5-dimethylphenyl)-1-oxopentan-2-yl)amino)-3-(4′-((1-((1R,8S,9s)-bicyclo[6.1.01 non-4-yn-9-yl)-3,17-dioxo-2,7,10,13,16-pentaoxa-4,18-diazadocosan-22-yl)oxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (LP5)

To a solution of P9 (12 mg, 7.76 μmol, 1 eq.) in DMF (0.5 mL) were added L6 (8.30 mg, 15.53 μmol, 2.0 eq.) and TEA (1.57 mg, 15.53 μmol, 2.16 μL, 2.0 eq.). Then the mixture was stirred at 20° C. for 12 h. LCMS trace showed that most of reactant was consumed completely and the desired MS was detected. It was purified by prep-HPLC (column: Phenomenex Gemini-NX 150*30 mm*5 μm; mobile phase: [water (0.225% FA)-ACN]; B %: 0%-55%, 45 min). Compound LP5 (4 mg, 2.00 μmol, 25.75% yield, 97% purity) was obtained as a white solid.

LCMS: (ESI): Rt=4.087 min, mass calcd. for C₉₅H₁₃₂FN₁₉O₂₄ [M+2H]²+971.5, m/z found 971.5 [M+2H]²⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC: (ESI): Rt=10.12 min, Reverse phase LCMS was carried out using Column: YMC-Pack ODS-A 150*4.6 mm, with a flow rate of 1.5 ml/min, eluting with a gradient of 10% to 80% acetonitrile containing 0.02% TFA (solvent B) and water containing 0.04% TFA (solvent A).

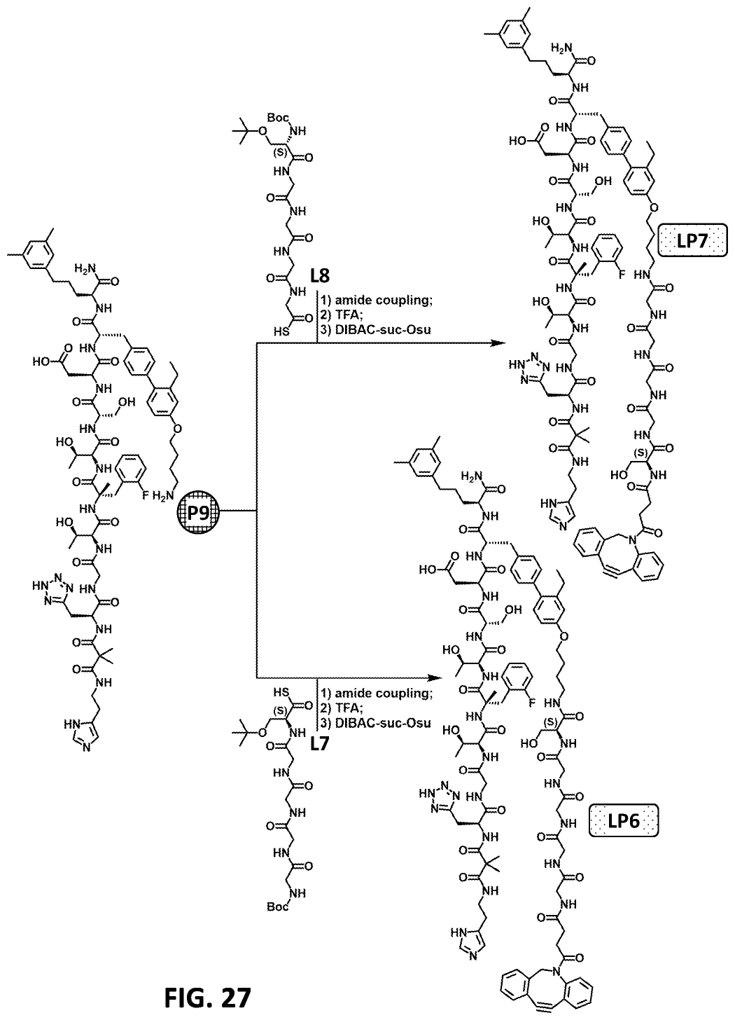

FIG. 27 depicts synthesis of linker-payloads LP6 and LP7 according to the disclosure.

### 5.6 Preparation of (3S)-4-[[(1S)-1-[[4-[4-[4-[[(2S)-2-[[2-[[2-[[2-[[2-[[4-(128- azatricyclohexadeca-8(14),9(15),10(16),12(18),63,65(68)-hexaen-33-yn-128-yl)-4-oxo-butanoyl]amino]acetyl]amino]acetyl]amino]acetyl]amino]acetyl]amino]-3-hydroxy-propanoyl]amino]butoxy]-2-ethyl-67-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl) ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP6)

To an oven-dried vial were charged L7 (163.54 mg, 323.48 μmol, 2.5 eq.) and P9 (200 mg, 129.39 μmol, 1 eq.). A stock solution of HOBt (69.93 mg, 517.56 μmol, 4 eq.), DIPEA (50.17 mg, 388.17 μmol, 3 eq.) in DMF (1 mL) and 12 (39.41 mg, 155.27 μmol, 1.2 eq.) in DMF (1 mL) was added to the vial. The reaction mixture was stirred at 15° C. for 12 h. The reaction progress was monitored by LCMS trace. The mixture was filtered, then precipitated by added EtOAc (20 mL). After filtration, the crude product protected G4S—P9 (261 mg, 116.45 μmol, 90.00% yield, 90% purity) was obtained as a pale-yellow foam.

LCMS (ESI): RT=3.283 min, mass calcd. for C₉₅H₁₃₆FN₂₃O₂₅²⁺1009.005 [M+2H-Boc]²⁺, m/z found 1009.5 [M+2H]²⁺. LCMS conditions: Flash RP-18e 25-2 mm, with a flow rate of 0.8 mL/min, eluting with a gradient of 10% to 80% acetonitrile containing 0.02% TFA (solvent B) and water containing 0.04% TFA (solvent A).

To an oven-dried vial was charged protected G4S—P9 (261 mg, 129.39 μmol, 1 eq.) and DCM (5 mL). TFA (6.70 g, 58.75 mmol, 4.35 mL, 454.08 eq.) was added to the vial. The reaction mixture was stirred at 20° C. for 2 h. The reaction progress was monitored by LCMS. The reaction mixture was concentrated and purified by prep-HPLC (TFA condition; column: Waters Xbridge Prep OBD C18 150*40 mm*10 μm; mobile phase: [water (0.1% TFA)-ACN]; B %: 0%-70%, 30 min). G4S—P9 (206 mg, 98.61 μmol, 76.21% yield, 100% purity, 2 TFA) was obtained as a white foam.

LCMS (ESI): RT=2.497 min, mass calcd. for C₉₅H₁₃₆FN₂₃O₂₅²⁺930.945 [M+2H]²⁺, m/z found 931.0 [M+2H]²⁺. LCMS conditions: Chromolith Flash RP-C18 25-3 mm, with a flow rate of 1.5 mL/min, eluting with a gradient of 10% to 80% acetonitrile containing 0.02% TFA (solvent B) and water containing 0.04% TFA (solvent A).

To an oven-dried vial were charged DIBAC-suc-OSu (39.68 mg, 98.61 μmol, 1 eq.) and G4S—P9 (206 mg, 98.61 μmol, 1 eq., 2 TFA). DMF (2 mL) and DIPEA (38.23 mg, 295.83 μmol, 3 eq.) were added to the vial. The reaction mixture was stirred at 20° C. for 1 h. The reaction progress was monitored by LC-MS. The reaction was fitered and purified by prep-HPLC (neutral condition; column: Waters Xbridge Prep OBD C18 150*40 mm*10 μm; mobile phase: [water (10 mM NH₄HCO₃)-ACN]; B %: 0%-50%, 30 min.). LP6 (112 mg, 52.13 μmol, 52.87% yield, 100% purity) was obtained as a white foam.

LCMS (ESI): RT=2.419 min, mass calcd. for C₁₀₅H₁₃₃FN₂₄O₂₅²⁺1074.49 [M+2H]²⁺, m/z found 1074.8 [M+2H]²⁺. LCMS conditions: Waters Xbridge C18 30*2.0 mm, 3.5 μm Mobile phase: A) 0.05% NH3H2O in Water; B) ACN. Gradient: 0% B increase to 95% B within 5.8 min; hold at 95% B for 1.1 min; then back to 0% B at 6.91 min and hold for 0.09 min. Flow rate 1.0 mL/min.

HPLC: RT=3.58 min. HPLC conditions: Mobile Phase: 0.2 ML/1 L NH₃·H₂O in water (solvent A) and acetonitrile (solvent B),using the elution gradient 0%-60% (solvent B) over 5 minutes and holding at 60% for 2 minutes at a flow rate of 1.2 ml/min; Column: Xbridge Shield RP-18, 5 μm, 2.1*50 mm.

¹H NMR (400 MHz, ACETONITRILE-d₃) δ ppm 8.40 (s, 1H) 7.52 (brt, J=6.82 Hz, 2H) 7.35-7.46 (m, 3H) 7.27-7.34 (m, 2H) 7.19-7.25 (m, 1H) 7.05-7.15 (m, 4H) 6.95 (br d, J=5.75 Hz, 4H) 6.86-6.91 (m, 1H) 6.74-6.82 (m, 5H) 6.70 (br d, J=8.13 Hz, 1H) 4.98 (br d, J=14.51 Hz, 1H) 4.61 (br s, 1H) 4.38-4.46 (m, 2H) 4.29 (br s, 2H) 4.16-4.21 (m, 1H) 4.09-4.12 (m, 2H) 3.89-4.01 (m, 6H) 3.79-3.88 (m, 10H) 3.57-3.77 (m, 7H) 3.28-3.36 (m, 3H) 3.16 (br s, 4H) 3.09 (br s, 1H) 2.82-2.89 (m, 1H) 2.76 (br d, J=3.75 Hz, 2H) 2.61-2.70 (m, 1H) 2.37-2.48 (m, 6H) 2.16 (s, 6H) 1.53-1.81 (m, 8H) 1.28 (br d, J=3.38 Hz, 3H) 1.17-1.22 (m, 6H) 1.11 (br d, J=6.00 Hz, 6H) 0.92 (br t, J=7.44 Hz, 3H).

### 5.7 Preparation of (3S)-4-[[(1S)-1-[[4-[4-[4-[[2-[[2-[[2-[[2-[[(2S)-2-[[4-(128- azatricyclohexadeca-8(14),9(15),10(16),12(18),63,65(68)-hexaen-33-yn-128-yl)-4-oxo-butanoyl]amino]-3-hydroxypropanoyl]amino]acetyl]amino]acetyl]amino]acetyl]amino]acetyl]amino]butoxy]-2-ethyl-67-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (LP7)

Starting from L8 (50 mg, 32.35 μmol, 1 eq.) and P9 (32.71 mg, 64.70 μmol, 2 eq.), LP7 (15 mg, 6.74 μmol, 53.21% yield, 96.47% purity) was obtained as a white solid using the same procedure as described in Example 6.

LCMS: (ESI): RT=0.845 min, m/z calcd. for C₁₀₅H₁₃₃FN₂₄O₂₅, 1075.5 [M+2H]²⁺, m/z found 1074.6 [M+2H]²⁺; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=3.55 min. Mobile Phase: Mobile Phase: 0.5 ML/1 L NH3H2O in water (solvent A) and acetonitrile (solvent B), using the elution gradient 0%-60% (solvent B) over 5 minutes and holding at 60% for 2 minutes at a flow rate of 1.2 ml/min.

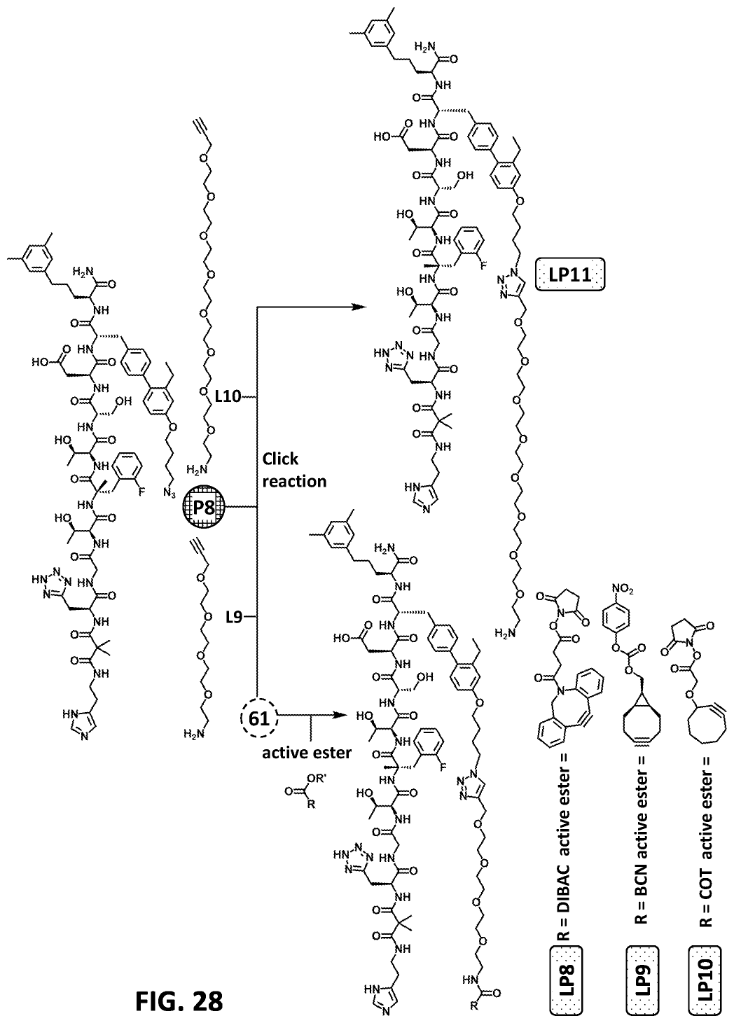

FIG. 28 depicts synthesis of linker-payloads LP8, LP9, LP10 and LP11 according to the disclosure.

### 5.8 Preparation of (3S)-4-[[(1S)-1-[[4-[4-[4-[4-[2-[2-[2-[2-[[4-(2-azatricyclo[10.4.0.04,91hexadeca- 1(12)₄(9),5,7,13,15-hexaen-10-yn-2-yl)-4-oxobutanoyl]amino]ethoxy]ethoxy]ethoxyl ethoxymethyl]triazol-1-yl]butoxy]-2-ethyl-phenyl]phenyl]methyl]-2-[[(1S)-carbamoyl-4-(3,5-dimethylphenyl)butyl]amino]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (LP8)

To a solution of P8 (20 mg, 12.73 μmol, 1 eq.) and L9 (8.83 mg, 38.18 μmol, 3.0 eq.) in H₂O (0.25 mL) and t-BuOH (0.5 mL) were added CuSO₄·5H₂O (635.45 μg, 2.55 μmol, 0.2 eq.) and sodium;(2R)-2-[(1S)-1,2-dihydroxyethyl]-4-hydroxy-5-oxo-2H-furan-3-olate (1.01 mg, 5.09 μmol, 0.4 eq). The mixture was stirred at 20° C. for 1.5 h. LCMS trace showed the material was disappeared and the desired product was observed as the major. The green solution was filtered to give the crude product. The crude product was purified by reversed phase HPLC (ISCO@; 20 g C18@ Silica Flash Column, Eluent of 0˜60.1% CH₃CN/H₂O (0.4% AcOH) gradient @ 18 mL/min). The desired fluent was lyophilized in freeze dryer to give compound 61 (15 mg, 8.24 μmol, 64.78% yield, 99.090% purity) as a white solid.

LCMS (ESI): RT=0.764 min, mass calcd. for C₂₃H₄₃N₁₁O₆ 901.95, m/z found 902.3 [M+H]⁺. LC-MS method A: a Xtimate C18 2.1*30 mm, 3 μm column, with a flow rate of 1.2 mL/min, eluting with a gradient of 5% to 95% acetonitrile containing 0.75 ML/4 L TFA (solvent B) and 1.5 ML/4 L TFA in water (solvent A).

To a solution of 61 (17 mg, 9.43 μmol, 1 eq.) and DIBAC-suc-OSu (5.05 mg, 12.55 μmol, 1.33 eq.) in DMF (2 mL) was added DIPEA (2.44 mg, 18.86 μmol, 3.28 μL, 2.0 eq.). The solution was stirred at 15° C. for 1 h. LCMS showed the reaction was converted completely and the desired product was observed. The solution was filtered and purified by prep-HPLC (neutral condition: Column: Durashell C18(L) 100*10 mm*5 μm; Mobile phase: [water (10 mM NH4HCO3)-ACN]; B %: 12%-52%, 12 min). The desired fluent was lyophilized in freeze dryer to give LP8 (7.0 mg, 3.21 μmol, 30.42% yield, 95.745% purity) as a white solid.

LCMS (ESI): RT=1.337 min, m/z calcd. for C₁₀₅H₁₃₅FN₂₂O₂₃ [M+2H]²+1045.50, found 1046.1. LCMS conditions: 0.8 mL/4 L NH₃·H20 in water (solvent A) and acetonitrile (solvent B), using the gradient 10%-80% (solvent B) over 2 minutes and holding at 80% for 0.48 minutes at a flow rate of 1 ml/min; Column: XBridge C18 3.5 μm 2.1*30 mm; Wavelength: UV 220 nm&254 nm; Column temperature: 50° C.

HPLC: RT=2.868 min, Mobile Phase: 0.2 mL/1 L NH₃·H20 in water (solvent A) and acetonitrile (solvent B), using the elution gradient 10%-80% (solvent B) over 6 minutes and holding at 80% for 2 minutes at a flow rate of 0.8 ml/min; Column: Xbridge Shield C18, 5 μm, 2.1*50 mm;

### 5.9 Preparation of (3S)-4-[[(1S)-1-[[4-[4-[4-[4-[2-[2-[2-[2-[[(1S,8R)-9-bicyclo[6.1.0]non-4-ynyl]methoxycarbonylamino]ethoxy]ethoxy]ethoxy]ethoxymethyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[3-(2-fluorophenyl)-2-[[(2S,3R)-3-hydrox-2-[[2-[[(2S)-2-[[3-[2-(1H-imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP9)

To a solution of compound 61 (14.55 mg, 8.07 μmol, 1 eq.) in DMF (2.0 mL) were added (1R,8S,9s)-bicyclo[6.1.0]non-4-yn-9-ylmethyl (4-nitrophenyl) carbonate (BCN—CO—PNP) (5.81 mg, 18.41 μmol, 2.28 eq.) and DIPEA (2.09 mg, 16.14 μmol, 2.81 μL, 2.0 eq.). The solution was purified by prep-HPLC (FA condition) (Column: Phenomenex Gemini-NX 150*30 mm*5 μm; mobile phase: [water (0.225% FA)-ACN]; B %: 0%-55%, 45 min) to give LP9 (3.6 mg, 1.79 μmol, 22.21% yield, 98.54% purity) was obtained as a white solid.

LCMS (ESI): RT=4.040 min, mass calcd. for C₉₇H₁₃₄FN₂₁O₂₃ [M+2H]²+989.89, found 990.6. LCMS conditions: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate 3 μm, C18,2.1*30 mm;

HPLC: RT=9.89 min, Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm,5 μm.

### 5.10 Preparation of (3S)-4-[[(1S)-2-[[(1S)-1-carbamoyl-4-(3,5-dimethylphenyl)butyl]amino]-1-[[4-[4-[4-[4-[2-[2-[2-[2-[(2-cyclooct-2-yn-1-yloxyacetyl)amino]ethoxy]ethoxy]ethoxy]ethoxymethyl]triazol-1-yl]butoxy]-2-ethyl-phenyl]phenyl]methyl]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP10)

To a solution of 61 (14 mg, 7.76 μmol, 1 eq) in DMF (0.5 mL) were added 2,5-dioxopyrrolidin-1-yl 2-(cyclooct-2-yn-1-yloxy)acetate (5.18 mg, 18.53 μmol, 2.39 eq) and DIPEA (2.01 mg, 15.53 μmol, 2.70 μL, 2.0 eq). The solution was stirred at 20° C. for 1 hr. LCMS showed the reaction was converted completely and the desired product was observed. The mixture was diluted with CH3CN, and the crude product was purified by prep-HPLC (FA condition: Column: Phenomenex Gemini-NX 150*30 mm*5 μm; Mobile phase: [water (0.225% FA)-ACN]; B %: 5%-55%, 35 min). The desired fluent was lyophilized in freeze dryer to give LP10 (2.6 mg, 1.26 μmol, 13.08% yield, 95.26% purity) as a white solid.

LCMS (ESI): RT=3.805 min, m/z calcd. for C₉₆H₁₃₄FN₂₁O₂₃ [M+2H]²+983.99, found 984.7. LCMS conditions: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate 3 μm, C18,2.1*30 mm.

HPLC: RT=9.74 min, Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm,5 μm; HPLC: RT=9.89 min, Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm.

### 5.11 Preparation of (3S)-4-[[(1S)-1-[[4-[4-[4-[4-[2-[2-[2-[2-[2-[2-[2-(2-aminoethoxy)ethoxy]ethoxy]ethoxy]ethoxy]ethoxy]ethoxy]ethoxymethyl]triazol-1-yl]butoxy]-2-ethyl-phenl]phenl]methyl]-2-[[(1S)-1-carbamoyl-4-(3,5-dimethylphenyl)butyl]amino]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl) ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (LP11)

A mixture of P8 (10 mg, 6.36 μmol, 1 eq.), sodium ascorbate (504.18 μg, 2.54 μmol, 0.4 eq.) and CuSO₄·5H₂O (317.72 μg, 1.27 μmol, 0.2 eq.) in t-BuOH (0.8 mL) and H₂O (0.4 mL) were stirred at 20° C. for 2 h. LCMS showed P8 was consumed completely. The reaction was filtered to give a residue. The residue was purified by prep-HPLC (column: Phenomenex Genimi NX C18 150*40 mm*5 μm; mobile phase: [water (0.1% TFA)-ACN]; B %: 0%-60%, 25 min) to give LP11 (4.32 mg, 2.18 μmol, 34.31% yield) was obtained as a white solid.

LCMS: (ESI): RT=3.121 min, m/z calcd. for C₉₄H₁₃₈FN₂₁O₂₅, 990.01 [M+2H]²⁺, m/z found 990.6 [M+2H]²⁺; Mobile Phase: Mobile Phase: 1.5 ML/4 L TFA in water (solvent A) and 0.75 ML/4 L TFA in acetonitrile (solvent B),using the elution gradient 10%-80% (solvent B) over 6 minutes and holding at 80% for 0.5 minutes at a flow rate of 0.8 ml/min.

HPLC (ES8584-1120-P1C1) was attached. RT=3.75 min, 99.72% purity. HPLC method: Column: Ultimate XB-C18.3 μm, 3.0*50 mm; 2.75 ML/4 L TFA in water (solvent A) and 2.5 ML/4 L TFA in acetonitrile (solvent B), using the elution gradient 10%-80% (solvent B) over 7 minutes and holding at 80% for 0.5 minutes at a flow rate of 1.5 ml/min.

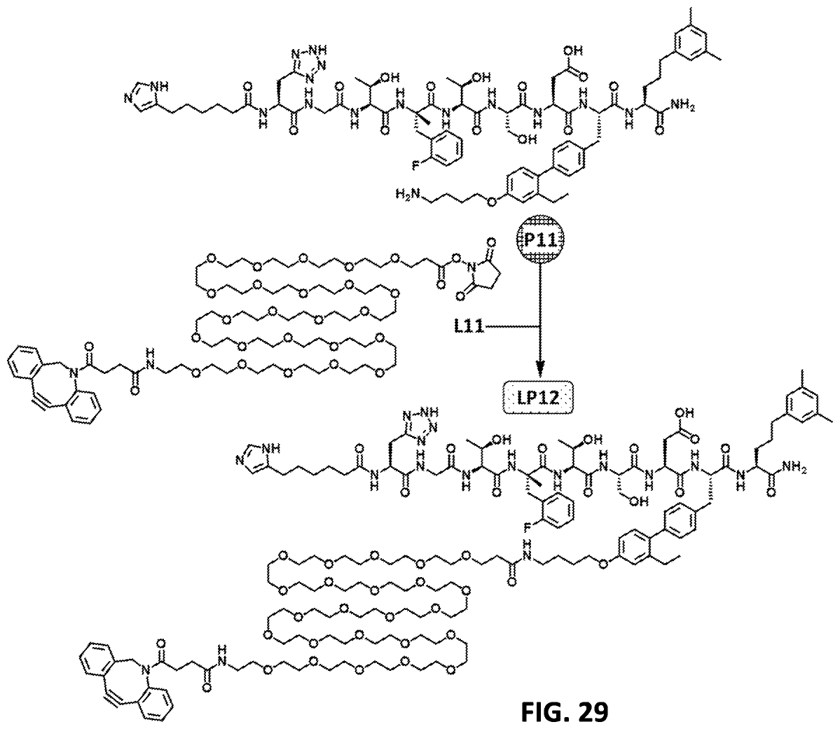

FIG. 29 depicts synthesis of linker-payload LP12 according to the disclosure.

### 5.12 Preparation of (3S)-4-[[(1S)-1-[[4-[4-[4-[3-[2-[2-[2-[2-[2-[2-[2-[2-[2-[2-[2-[2-[2-[2-[2-[2-[2-[2-[2-[2-[2-[2-[2-[2-[[4-(162-azatricyclohexadeca-6(12),7(13),8(14),10(16),109,111(114)-hexaen-31-yn-162-yl)-4-oxo-butanoyl]amino]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]eth oxy]ethoxy]ethoxy]propanoylamino]butoxy]-2-ethyl-113-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[6-(1H-imidazol-5-yl) hexanoylamino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP12)

To a solution of P11 (30 mg, 14.65 μmol, 1 eq., TFA) and L11 (22.43 mg, 14.65 μmol, 1 eq.) in DMF (1.5 mL) was added DIPEA (9.47 mg, 73.25 μmol, 12.76 μL, 5 eq.) at 15° C. The mixture was stirred at 15° C. for 1 h. LCMS trace showed that the reaction was complete. The mixture was filtered to give crude as yellow oil, and the residue was purified by prep-HPLC (column: Waters Xbridge Prep OBD C18 150*40 mm*10 μm; mobile phase: [water (10 mM NH₄HCO₃)-ACN]; B %: 0%-80%, 30 min) to give LP12 (25 mg, 8.18 μmol, 47.72% yield, 95.49% purity) as a white solid.

LCMS: (ESI): RT=0.970 min, m/z calcd. for C₁₄₄H₂₁₇FN₁₉O₄₃, 973.18 [M+3H]³*, m/z found 974.0 [M+3H]³⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC: Rt=2.53; Mobile Phase: 0.2 ML/1 L NH₃H2O in water (solvent A) and acetonitrile (solvent B), using the elution gradient 10%-80% (solvent B) over 5 minutes and holding at 80% for 2 minutes at a flow rate of 1.2 ml/min Column: Xbridge Shield RP-18.5 μm, 2.1*50 mm.

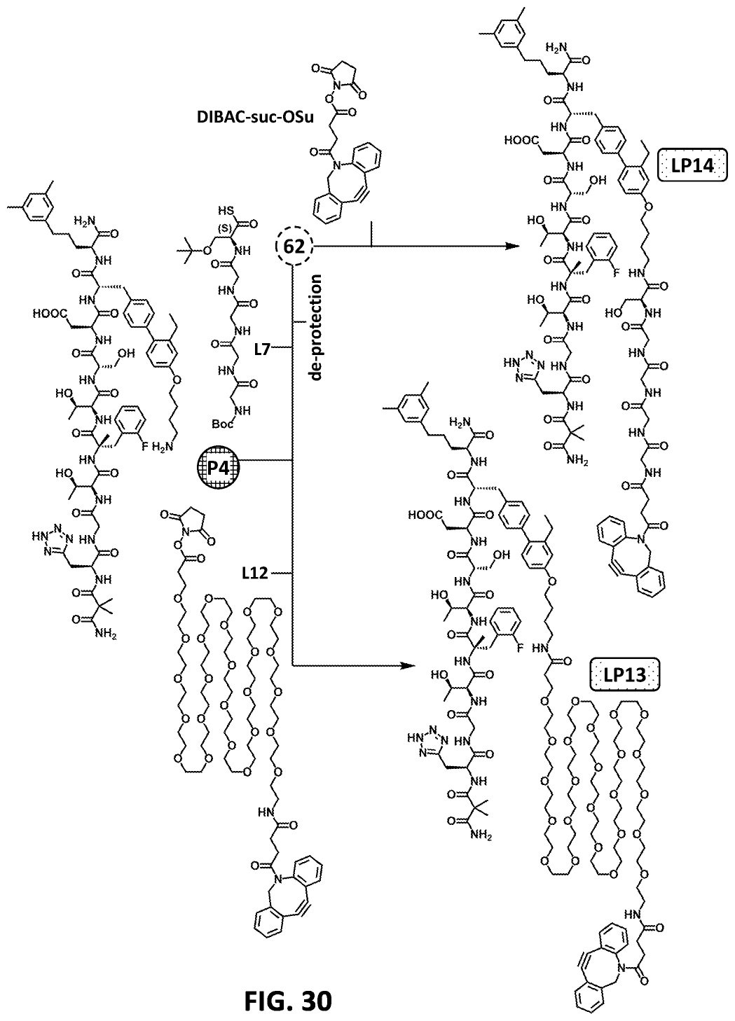

FIG. 30 depicts synthesis of linker-payloads LP13 and LP14 according to the disclosure.

### 5.13 Preparation of (3S)-3-[[(2S)-2-[[(2S,3R)-2-[[(2S)-2-[[(2S,3R)-2-[[2-[[(2S)-2-[(3-amino-2,2-dimethyl-3-oxo-propanoyl)amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-[[(1 S)-1-[[4-[4-[4-[3-[2-[2-[2-[2-[2-[2-[2-[2-[2-[2-[2-[2-[2-[2-[2-[2-[2-[2-[2-[2-[2-[2-[2-[2-[[4-(157-azatricyclohexadeca-8(14),9(15),10(16),12(18),104, 106(109)-hexaen-31-yn-157-yl)-4-oxobutanoyl]amino]ethoxy]ethoxy]ethoxy]ethoxy]ethoxyl ethoxy]ethoxy]ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]propanoylamino]butoxy]-2-ethyl-108-phenyl]phenyl]methyl]-2-[[(1S)-1-carbamoyl-4-(3,5-dimethylphenyl)butyl]amino]-2-oxo-ethyl]amino]-4-oxo-butanoic acid (LP13)

To a solution of P4 (10 mg, 6.89 μmol, 1 eq.) and L12 (10.55 mg, 6.89 μmol, 1 eq.) in DMF (0.5 mL) was added DIPEA (4.45 mg, 34.44 μmol, 6.00 μL, 5 eq.) at 15° C. The mixture was stirred at 15° C. for 1 h. LCMS showed that the reaction was converted completely. The mixture was filtered to give crude as a yellow oil, which was purified by prep-HPLC (column: Waters Xbridge Prep OBD C18 150*30 mm, 10 μm; mobile phase: [water(10 mM NH₄HCO₃)-ACN]; B %: 10%-50%, 55 min) to give LP13 (11 mg, 3.84 μmol, 36.67% yield) as a white solid.

LCMS: (ESI): RT=0.965 min, m/z calcd. for C₁₄₀H₂₁₃FN₁₈O₄₄, 717.38 [M+4H]²⁺, m/z found 717.8 [M+4H]⁴⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC: RT=3.97; Mobile Phase: 0.2 ML/1 L NH₃H₂O in water (solvent A) and acetonitrile (solvent B), using the elution gradient 0%-60% (solvent B) over 5 minutes and holding at 60% for 2 minutes at a flow rate of 1.2 ml/minColumn: Xbridge Shield RP-18.5 μm, 2.1*50 mm Wavelength: 220 nm&215 nm&254 nm.

### 5.14 Preparation of (3S)-3-[[(2S)-2-[[(2S,3R)-2-[[(2S)-2-[[(2S,3R)-2-[[2-[[(2S)-2-[(3-amino-2,2-dimethyl-3-oxo-propanoyl)amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-[[(1S)-1-[[4-[4-[4-[[(2S)-2-[[2-[[2-[[2-[[2-[[4-(121-azatricyclohexadeca-8(14),9(15),10(16),12(18),59,61(64)-hexaen-31-yn-121-yl)-4-oxo-butanoyl]amino]acetyl]amino]acetyl]amino]acetyl]amino]acetyl]amino]-3-hydroxy-propanoyl]amino]butoxy]-2-ethyl-63-phenyl]phenyl]methyl]-2-[[(1S)-1-carbamoyl-4-(3,5-dimethylphenyl)butyl]amino]-2-oxo-ethyl]amino]-4-oxo-butanoic acid (LP14)

To a solution of P4 (40 mg, 27.56 μmol, 1 eq.) in DMF (0.5 mL) were added HOBt (14.89 mg, 110.22 μmol, 4 eq.), DIPEA (10.68 mg, 82.67 μmol, 14.40 μL, 3 eq.) and L7 (34.83 mg, 68.89 μmol, 2.5 eq.); then I₂ (8.39 mg, 33.07 μmol, 6.66 μL, 1.2 eq.) in DMF (0.5 mL) was added. The reaction mixture was stirred at 20° C. for 16 hr. To the reaction was added EtOAc (15 mL) and white solids were precipitated. The mixture was centrifuged for 3 min (5000 R) to get the solid. Then the solid was dissolved in H₂O (10 mL) and CAN (2 mL). The solution was lyophilized to give the crude protected compound 62 (59 mg, 23.01 μmol, 83.50% yield, 75% purity) as a white solid.

LCMS (ESI): RT=3.882 min, mass calcd. for C₉₀H₁₃₀FN₂₁O₂₅ 1923.94 961.9[M+2H]²⁺, m/z found 962.5 [M+2H]²⁺; Mobile Phase: 1.5 ML/4 L TFA in water (solvent A) and 0.75 ML/4 L TFA in acetonitrile (solvent B),using the elution gradient 10%-80% (solvent B) over 3 minutes and holding at 80% for 0.5 minutes at a flow rate of 0.8 ml/min; Column: Xtimate C18 2.1*30 mm, 3 μm; Wavelength: UV 220 nm; Column temperature: 50° C.; MS ionization: ESI

To a solution of protected compound 62 (59 mg, 30.68 μmol, 1 eq.) in DCM (0.5 mL) was added TFA (0.5 mL) at 0° C. Then the mixture was warmed to 20° C. and stirred at 20° C. for 2 hr. LCMS trace showed the reactant was consumed completely and the desire MS was detected. The solvent was removed under reduced pressure at 30° C. to give the crude. The residue was purified by prep-HPLC (column: Phenomenex Gemini-NX C18 75*30 mm*3 μm; mobile phase: [water (0.075% TFA)-ACN]; B %: 0%-60%, 35 min.) to give compound 62 (11 mg, 5.91 μmol, 19.28% yield, 95% purity) as a white solid.

LCMS (ESI): RT=2.936 min, mass calcd. for C₈₁H₁₁₄FN₂₁O₂₃ 1767.82 884.2 [M+2H]²⁺, m/z found 884.2 [M+2H]²⁺; Mobile Phase: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min. ESI source, Positive ion mode; Wavelength 220 nm&254 nm, OvenTemperature 50° C.

HPLC: RT=6.05 min Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm; Wavelength: UV 220 nm&215 nm&254 nm; Column temperature

To a solution of compound 62 (11 mg, 6.23 μmol, 1 eq.) and DIBAC-suc-OSu (2.76 mg, 6.85 μmol, 1.1 eq.) in DMF (0.5 mL) was added DIPEA (4.02 mg, 31.13 μmol, 5.42 μL, 5.0 eq.) at 20° C. Then the mixture was stirred at 20° C. for 2 hr. LCMS trace showed the reactant was consumed completely and the desired MS was detected. It was purified by prep-HPLC (column: Phenomenex Gemini-NX C18 75*30 mm*3 μm; mobile phase: [water (10 mM NH₄HCO₃)-ACN]; B %: 0%-60%, 35 min). LP14 (10 mg, 4.62 μmol, 74.28% yield, 95% purity) was obtained as a white solid.

LCMS (ESI): RT=0.939 min, mass calcd. for C₁₀₀H₁₂₇FN₂₂O₂₅ 2054.92, m/z found 1028.0 [M+2H]²⁺ Mobile Phase: 1.5 mL/4 L TFA in water (solvent A) and 0.75 mL/4 L TFA in acetonitrile (solvent B), using the elution gradient 5%-95% (solvent B) over 0.7 minutes and holding at 95% for 0.4 minutes at a flow rate of 1.5 mL/min; Column: Agilent Pursult 5 C18 20*2.0 mm Wavelength: UV 220 nm; Column temperature: 50° C.

HPLC: RT=3.75 min Mobile Phase: water (solvent A) and acetonitrile (solvent B), using the elution gradient 0%-60% (solvent B) over 5 minutes and holding at 60% for 2 minutes at a flow rate of 1.2 ml/min; Column: Xbridge Shield RP-18, 5 μm, 2.1*50 mm; Wavelength: UV 220 nm, 215 nm&254 nm; Column temperature: 40° C.

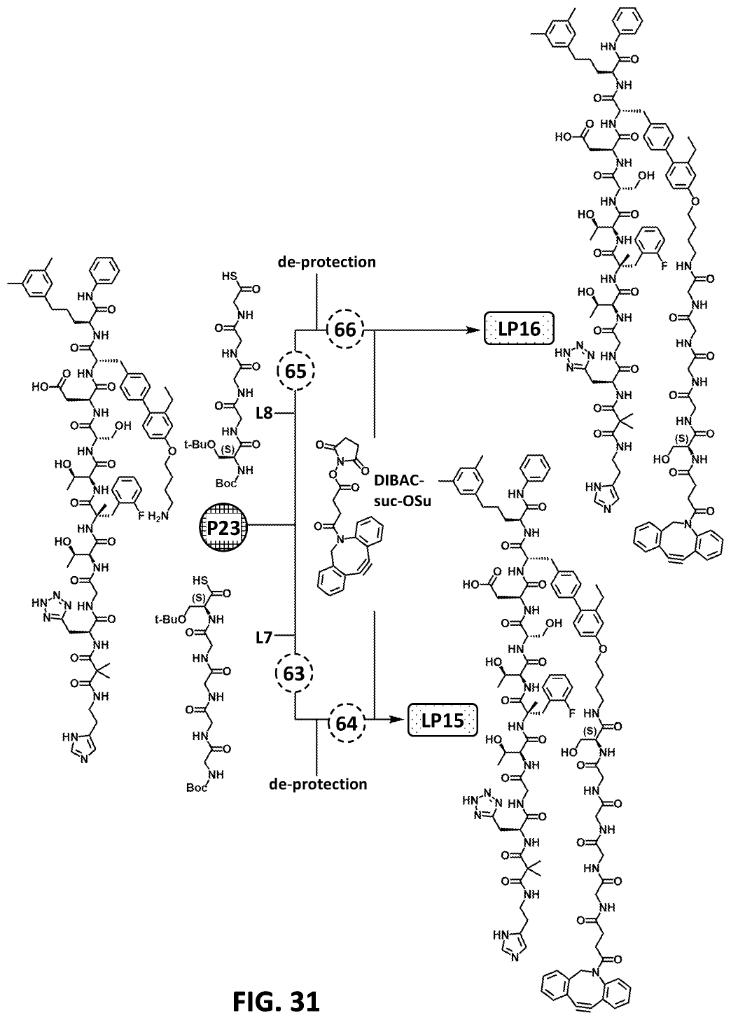

FIG. 31 depicts synthesis of linker-payloads LP15 and LP16 according to the disclosure.

### 5.15 Preparation of (3S)-4-[[(1S)-1-[[4-[4-[4-[[(2S)-2-[[2-[[2-[[2-[[2-[[4-(134- azatricyclohexadeca-11(19),12(20),13(21),15(23),69,71(74)-hexaen-38-yn-134-yl)-4-oxobutanoyl]amino]acetyl]amino]acetyl]amino]acetyl]amino]acetyl]amino]-3-hydroxy-propanoyl]amino]butoxy]-2-ethyl-73-phenyl]phenyl]methyl]-2-[[(1S)-4-(3,5-dimethylphenyl)-1-(phenylcarbamoyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methylpropanoyl]amino]-3-hydroxy-butanoyl]amino]-3-hydroxy-propanoyl]amino]-4-oxo-butanoic acid (LP15)

To an oven-dried vial were charged L7 (47.33 mg, 93.62 μmol, 2.5 eq.) and P23 (65 mg, 37.45 μmol, 1 eq., TFA). A stock solution of HOBt (20.24 mg, 149.78 μmol, 4 eq.), DIPEA (14.52 mg, 112.34 μmol, 19.57 μL, 3 eq.) in DMF (0.5 mL) and 12 (11.40 mg, 44.94 μmol, 9.05 μL, 1.2 eq.) in DMF (2 mL) was added to the vial. The reaction mixture was stirred at 20° C. for 16 h. LCMS trace showed that the reaction was complete. The reaction was added EtOAc (15 mL) and white solids were precipitated. The mixture was centrifuged for 3 min (5000 R) to give the crude product 63 (60 mg, 27.82 μmol, 74.29% yield, and 97.06% purity) as a white solid.

LCMS (ESI): RT=0.940 min, mass calcd. for C₉₆H₁₃₂FN₂₃O₂₃ 997 m/z [M−Boc+3H]2+, m/z found 997.5 m/z [M−Boc+3H]2+; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

A solution of 63 (60 mg, 28.66 μmol, 1 eq.) in TFA (1 mL) and DCM (1 mL) were stirred at 20° C. for 2 h. LCMS trace showed that the reaction was complete. The reaction was concentrated in vacuum to give crude as yellow oil. The crude was purified by prep-HPLC (column: Waters Xbridge Prep OBD C18 150*40 mm*10 μm; mobile phase: [water (0.1% TFA)-ACN]; B %: 0%-60%, 30 min.) to give 64 (45 mg, 22.72 μmol, 79.27% yield, 97.81% purity) as a white solid.

LCMS (ESI): RT=0.890 min, mass calcd. for C₉₂H₁₂₄FN₂₃O₂₃ 968.96 m/z [M+2H]2+, m/z found 969.5 m/z [M+2H]2+; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC (ES8584-719-P1C1) was attached. RT=4.18 min., 97.81% purity. HPLC method A: Mobile phase: 1.0% ACN in water (0.1% TFA) to 5% ACN in water (0.1% TFA) in 1 min; then from 5% ACN in water (0.1% TFA) to 100% ACN (0.1% TFA) in 5 minutes; hold at 100% ACN (0.1% TFA) for 2 minutes; back to 1.0% CAN in water (0.1% TFA) at 8.01 min, and hold two minutes.Flow rate: 1.2 ml/min.

To a solution of 64 (40 mg, 19.50 μmol, 1 eq., TFA) and DIBAC-suc-OSu (7.85 mg, 19.50 μmol, 1 eq.) in DMF (1.5 mL) was added DIPEA (12.60 mg, 97.51 μmol, 16.98 μL, 5 eq.) at 18° C. The reaction was stirred at 18° C. for 2 h. LCMS trace showed that the reaction was complete. The crude was purified by prep-HPLC (column: Waters Xbridge Prep OBD C18 150*40 mm*10 μm; mobile phase: [water (10 mM NH4HCO3)-ACN]; B %: 0%-50%, 35 min.) to give LP15 (27 mg, 12.07 μmol, 55.75% yield, 99.42% purity) as a white solid.

LCMS (ESI): RT=0.948 min, mass calcd. for C₁₁₁H₁₃₇FN₂₄O₂₅ 1112.51 m/z [M+2H]²⁺, m/z found 1113.1 m/z [M+2H]²⁺; Reverse phase LC-MS was carried out using a Chromolith Flash RP-18e 25-3 mm column, with a flow rate of 1.5 mL/min, eluting with a gradient of 5% to 95% acetonitrile containing 0.02% TFA (solvent B) and water containing 0.04% TFA (solvent A).

HPLC: Reverse phase 0.2 ML/1 L NH₃·H₂O in water (solvent A) and acetonitrile (solvent B), using the elution gradient 10%-80% (solvent B) over 5 minutes and holding at 80% for 2 minutes at a flow rate of 1.2 ml/min Column: Xbridge Shield RP-18, 5 μm, 2.1*50 mm Wavelength: 220 nm&215 nm&254 nm, Column temperature: 40° C.

### 5.16 Preparation of (3S)-4-[[(1S)-1-[[4-[4-[4-[[2-[[2-[[2-[[2-[[(2S)-2-[[4-(134- azatricyclohexadeca-11(19),12(20),13(21),15(23),69,71(74)-hexaen-38-yn-134-yl)-4-oxo-butanoyl]amino]-3-hydroxypropanoyl]amino]acetyl]amino]acetyl]amino]acetyl]amino]acetyl]amino]butoxy]-2-ethyl-73-phenyl]phenyl]methyl]-2-[[(1S)-4-(3,5-dimethylphenyl)-1-(phenylcarbamoyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (LP16)

Starting from P23 (70 mg, 43.16 μmol, 1 eq), L8 (43.64 mg, 86.32 μmol, 2 eq) and DIBAC-suc-OSu (3.92 mg, 9.75 μmol, 1 eq), LP16 (10 mg, 4.25 μmol, 34.87% yield, 94.53% purity) as a white solid was prepared using the same procedure as described in Example 15.

LCMS: (ESI): RT=0.845 min, mass calcd. for C₁₁₁H₁₃₇FN₂₄O₂₅ 1112.51 [M+2H]²⁺, m/z found 1112.9[M+2H]²⁺; Mobile Phase: 0.8 mL/4 L NH₃·H₂O in water (solvent A) and acetonitrile (solvent B),using the elution gradient 10%-80% (solvent B) over 2 minutes and holding at 80% for 0.48 minutes at a flow rate of 1 ml/min;

HPLC: RT=3.73 min, 94.53% purity. Mobile Phase: 2.0 ML/4 L NH₃H₂O in water (solvent A) and Aetonitrile (solvent B), using the elution gradient 0%-60% (solvent B) over 4 minutes and holding at 60% for 2 minutes at a flow rate of 1.2 ml/min.

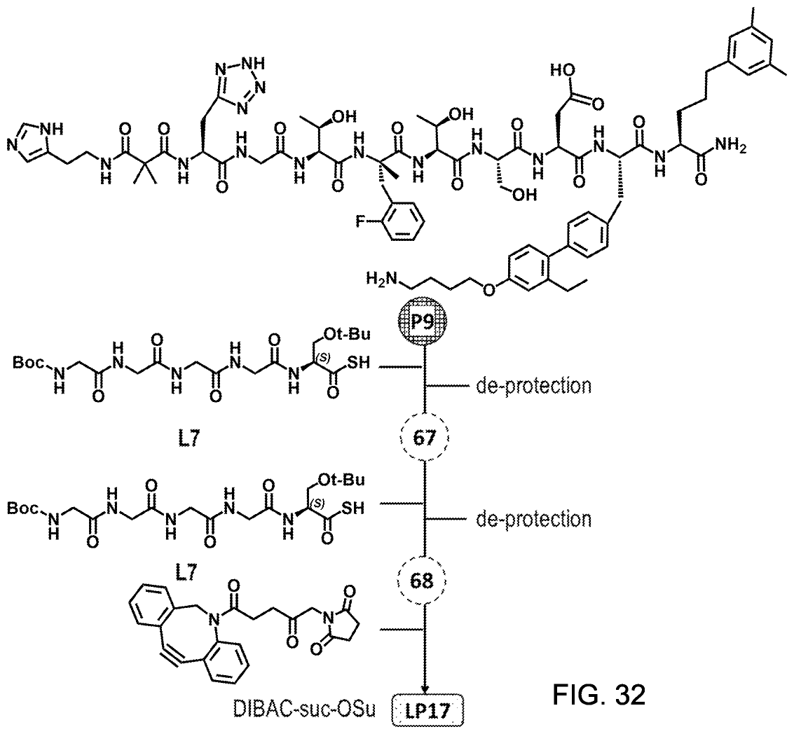

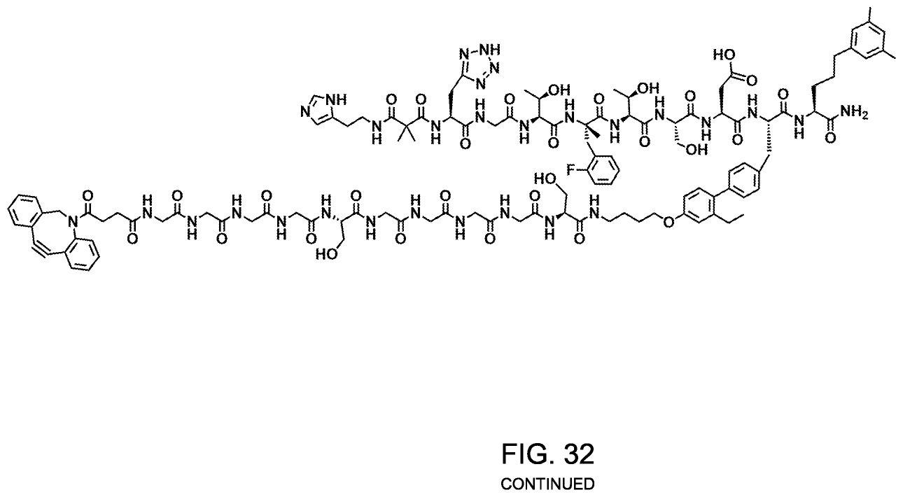

FIG. 32 depicts synthesis of linker-payload LP17 according to the disclosure.

### 5.17 Preparation of (3S)-4-[[(1S)-1-[[4-[4-[4-[[(2S)-2-[[2-[[2-[[2-[[2-[[(2S)-2-[[2-[[2-[[2-[[2-[[4-(144-azatricyclohexadeca-8(14),9(15),10(16),12(18),68,70(73)-hexaen-33-yn-144-yl)-4-oxo-butanoyl]amino]acetyl]amino]acetyl]amino]acetyl]amino]acetyl]amino]-3-hydroxy-propanoyl]amino]acetyl]amino]acetyl]amino]acetyl]amino]acetyl]amino]-3-hydroxy-propanoyl]amino]butoxy]-2-ethyl-72-phenyl]phenyl]methyl]-2-[[(1S)-1-carbamoyl-4-(3,5-dimethylphenyl)butyl]amino]-2-oxo-ethyl]amino]-3-[[(2S)-2-[[(2S, 3R)-2-[[(2S)-3-(2-fluorophenyl)-2-[[(2S, 3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP17)

To an oven-dried vial were charged L7 (26.17 mg, 51.76 μmol, 2 eq.), P9 (40.00 mg, 25.88 μmol, 1 eq.) and 4A MS (5 mg). A stock solution of HOBt (6.99 mg, 51.76 μmol, 2 eq.), DIPEA (5.02 mg, 38.82 μmol, 6.76 μL, 1.5 eq.) in DMF (0.1 mL) and 12 (3.94 mg, 15.53 μmol, 3.13 μL, 0.6 eq.) in DMF (0.1 mL) was added to the vial. The reaction mixture was stirred at 15° C. for 48 h. The reaction progress was monitored by LC-MS. The mixture was filtered, then precipitated by added EtOAc (3 mL). After filtration, protected G4S—P9 (50 mg, 22.31 μmol, 86.20% yield, 90% purity) was obtained as a white foam.

LCMS (ESI): RT=0.907 min, mass calcd. for C₉₅H₁₃₆FN₂₃O₂₅²⁺1009.005 [M+2H]²⁺, m/z found 1010.4 [M+2H]²⁺. LCMS conditions: a Merck, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=8.87 min. HPLC conditions: YMC-Pack ODS-A 150*4.6 mm, 5 mm column, flow rate 1.5 mL/min, eluting with a gradient of 10% to 80% acetonitrile containing 0.12% TFA (solvent B) and water containing 0.1% TFA (solvent A).

To an oven-dried vial were charged protected G4S—P9 (35 mg, 17.35 μmol, 1 eq.) and DCM (1 mL). TFA (1.54 g, 13.51 mmol, 1 mL, 778.42 eq.) was added to the vial. The reaction mixture was stirred at 20° C. for 3 h. The reaction progress was monitored by LC-MS. LCMS (ESI): RT=0.832 min, mass calcd. for C₉₅H₁₃₆FN₂₃O₂₅²⁺930.945 [M+2H]²⁺, m/z found 931.3 [M+2H]²⁺. LCMS conditions: a Merck, RP-18e 25-2 mm column, with a flow rate of 1.5 mL/min, eluting with a gradient of 5% to 95% acetonitrile containing 0.02% TFA (solvent B) and water containing 0.04% TFA (solvent A). The reaction mixture was concentrated to give the crude product 67 (35 mg, crude) as a yellow solid.

To an oven-dried vial were charged L7 (19.02 mg, 37.61 μmol, 2 eq.) and compound 67 (35 mg, 18.81 μmol, 1 eq.). A stock solution of HOBt (10.16 mg, 75.23 μmol, 4 eq.) and DIPEA (7.29 mg, 56.42 μmol, 9.83 μL, 3 eq.) in DMF (0.5 mL) and 12 (5.73 mg, 22.57 μmol, 4.55 μL, 1.2 eq.) in DMF (0.5 mL) was added to the vial. The reaction mixture was stirred at 15° C. for 24 h. The reaction progress was monitored by LC-MS. The mixture was filtered, then precipitated by added EtOAc (6 mL). After filtration, the crude product protected G4S-G4S—P9 (40 mg, 13.38 μmol, 71.12% yield, 78% purity) was obtained as a pale yellow foam.

LCMS (ESI): RT=3.400 min, mass calcd. for C₁₀₆H₁₅₃FN₂₈O₃₁²⁺1166.56, m/z found 1166.9 [M+2H]²⁺. LCMS conditions: a Merck, RP-18e 25-2 mm column, with a flow rate of 0.8 mL/min, eluting with a gradient of 10% to 80% acetonitrile containing 0.02% TFA (solvent B) and water containing 0.04% TFA (solvent A).

HPLC: RT=8.10 min. HPLC conditions: YMC-Pack ODS-A 150*4.6 mm, 5 mm column, flow rate 1.5 mL/min, eluting with a gradient of 10% to 80% acetonitrile containing 0.12% TFA (solvent B) and water containing 0.1% TFA (solvent A).

To an oven-dried vial were charged protected G4S-G4S—P9 (40 mg, 17.15 μmol, 1 eq.) and DCM (2 mL). TFA (3.08 g, 27.01 mmol, 2 mL) was added to the vial. The reaction mixture was stirred at 20° C. for 2 h. The reaction progress was monitored by LC-MS. The reaction was concentrated to give a residue, then purified by prep-HPLC (TFA condition; column: Waters Xbridge Prep OBD C18 150*30 mm, 10 μm; mobile phase: [water(0.1% TFA)-ACN]; B %: 0%-60%,16 min). Compound 68 (15 mg, 6.62 μmol, 38.58% yield, 96% purity) was obtained as a white foam.

LCMS (ESI): RT=0.744 min, mass calcd. for C₉₇H₁₃₇FN₂₈O₂₉²⁺1088.505 [M+2H]²⁺, m/z found 1089.2 [M+2H]²⁺. LCMS conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

HPLC: RT=3.89 min. HPLC conditions: Mobile phase:1.0% ACN in water (0.1% TFA) to 5% ACN in water (0.1% TFA) in 1 min; then from 5% ACN in water (0.1% TFA) to 100% ACN (0.1% TFA) in 5 minutes; hold at 100% ACN (0.1% TFA) for 2 minutes; back to 1.0% ACN in water (0.1% TFA) at 8.01 min, and hold two minutes.Flow rate:1.2 ml/min.

To a solution of DIBAC-suc-OSu (3.37 mg, 8.36 μmol, 1.3 eq.) in DMF (0.3 mL) was added compound 68 (14 mg, 6.43 μmol, 1 eq.) and DIPEA (4.16 mg, 32.17 μmol, 5.60 μL, 5 eq.). The mixture was stirred at 20° C. for 2 hr. The reaction progress was monitored by LC-MS. The reaction mixture was diluted with DMSO (2 mL) and purified by prep-HPLC (neutral condition, column: Waters Xbridge Prep OBD C18 150*40 mm*10 μm; mobile phase: [water(10 mM NH4HCO3)-ACN]; B %: 0%-60%,55 min). LP17 (1.73 mg, 0.65 μmol, 10.15% yield, 93% purity) was obtained as a white foam.

LCMS (ESI): RT=2.531 min, mass calcd. for C₁₁₆H₁₅₀FN₂₉O₁₃²⁺1232.05 [M+2H]²⁺, m/z found 1232.4 [M+2H]²⁺. LCMS conditions: Mobile phase: A) 0.05% NH₃H₂O in Water; B) ACN. Gradient: 0% B increase to 95% B within 5.8 min; hold at 95% B for 1.1 min; then back to 0% B at 6.91 min and hold for 0.09 min. Flow rate 1.0 mL/min; Column: Waters Xbridge C18 30*2.0 mm,3.5 μm.

HPLC: RT=2.17 min. HPLC conditions: Mobile Phase: 0.2 ML/1 L NH₃H₂O in water (solvent A) and acetonitrile (solvent B), using the elution gradient 10%-80% (solvent B) over 5 minutes and holding at 80% for 2 minutes at a flow rate of 1.2 ml/min; Column: Xbridge Shield RP-18.5 μm, 2.1*50 mm.

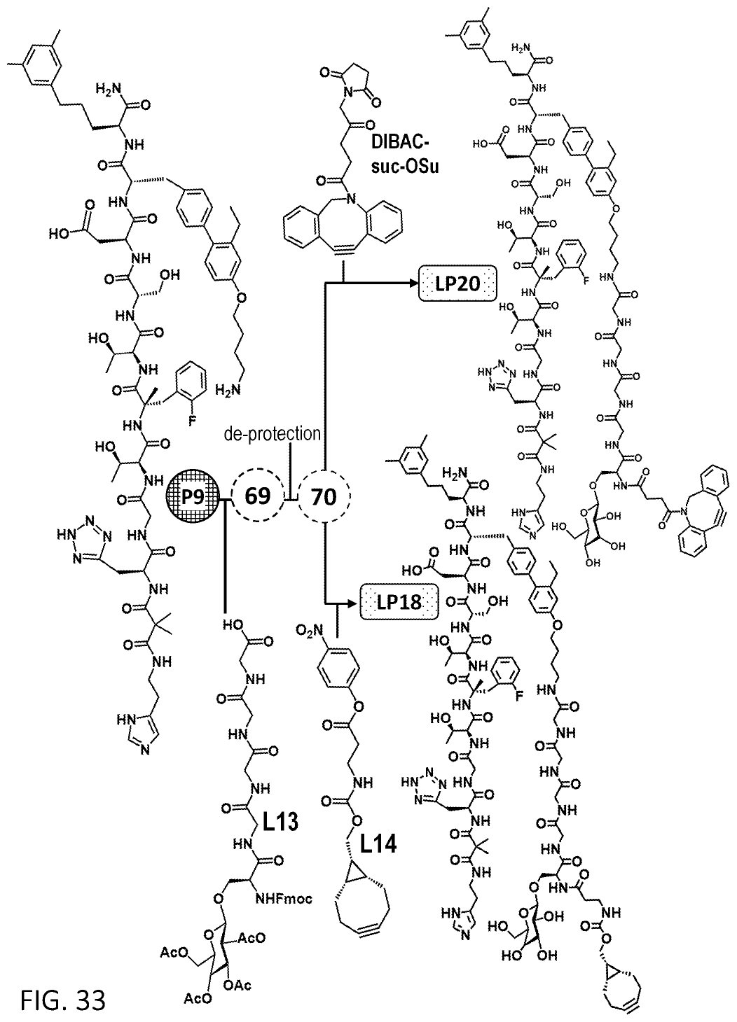

FIG. 33 depicts synthesis of linker-payloads LP18 and LP20 according to the disclosure.

### 5.18 Preparation of (3S)-4-[[(1S)-1-[[4-[4-[4-[[2-[[2-[[2-[[2-[[(2S)-2-[3-[[(1S,8R)- 9-bicyclo[6.1.01non-4-ynyl]methoxycarbony]amino]propanoylamino]-3-[(2R,4S,5S)-3,4,5-trihydroxy-6-(hydroxymethyl)tetrahydropyran-2-yl]oxypropanoyl]amino]acetyl]amino]acetyl]amino]acetyl]amino]acetyl]amino]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]asmino]-3-hydroxypropanoyl]amino]-4-oxo-butanoic acid (LP18)

To a solution of L13 (22.92 mg,25.88 μmol, 2 eq.), PyBOP (13.47 mg, 25.88 μmol, 2 eq.) and DIPEA (6.69 mg, 51.76 μmol, 9.01 μL, 4 eq.) in DMF (0.1 mL) was stirred at 25° C. for 5 min. A solution of P9 (20 mg, 12.94 μmol, 1 eq.) in DMF (0.1 mL) was added to the mixture, the reaction was stirred at 25° C. for 5 min. The mixture was diluted with EtOAc (15 mL) to give precipitate, which was centrifuged for 3 min (5000 R) to give the crude product as a white solid. The residue was diluted with water (2 mL) and ACN (2 mL). The solution was dried by lyophilization to give compound 69 (25 mg, 8.53 μmol, 65.95% yield, 82.384% purity) as a white solid.

LCMS: (ESI): RT=2.376 min, m/z calcd. for C₁₁₅H₁₄₈FN₂₃O₃₄, 1207.03 [M+2H]²⁺, m/z found 1207.4 [M+2H]²⁺; Mobile Phase: 1.5 ML/4 L TFA in water (solvent A) and 0.75 ML/4 L TFA in acetonitrile (solvent B),using the elution gradient 10%-80% (solvent B) over 3 minutes and holding at 80% for 0.5 minutes at a flow rate of 0.8 ml/min.

To a solution of compound 69 (20 mg, 8.29 μmol, 1 eq.) in DMF (2 mL) was added NH₂NH₂·H₂O (488.04 μg, 8.29 μmol, 0.2 mL, 85% purity, 1 eq.) at 25° C. The reaction was stirred at 25° C. for 1 h. LCMS showed that the reaction converted completely. The mixture was filtered to give a residue, which was purified by prep.-HPLC (column: Phenomenex Gemini-NX 150*30 mm*5 μm; mobile phase:[water(0.075% TFA)-ACN]; B %: 0%-60%,30 min) to give compound 70 (15 mg, 7.38 μmol, 71.28% yield, 99.58% purity) as a white solid.

LCMS: (ESI): RT=0.791 min, m/z calcd. for C₉₂H₁₃₀FN₂₃O₂₈, 1011.97 [M+2H]²⁺, m/z found 1012.2 [M+2H]²⁺; Reverse phase LCMS was carried out using a Merck RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: Rt=3.47 min. Mobile phase: 1.0% ACN in water (0.1% TFA) to 5% ACN in water (0.1% TFA) in 1 min; then from 5% ACN in water(0.1% TFA) to 100% ACN (0.1% TFA) in 5 minutes; hold at 100% ACN (0.1% TFA) for 2 minutes; back to 1.0% ACNin water (0.1% TFA) at 8.01 min, and hold two minutes. Flow rate: 1.2 ml/min.

A solution of compound 70 (18 mg, 8.90 μmol, 1 eq.) and L14 (6.88 mg, 17.79 μmol, 2 eq.) in PBS buffer (0.45 mL, pH=8.2) and DMF (0.9 mL) was stirred at 25° C. for 6 h. LCMS showed the reaction was complete. The reaction was filtered to give a residue. The residue was purified by prep-HPLC (column: Phenomenex Gemini-NX 150*30 mm*5 μm; mobile phase: [water (0.225% FA)-ACN]; B %: 0%-60%, 35 min) to give LP18 (7.5 mg, 3.24 μmol, 36.45% yield, 98.18% purity) as a white solid.

LCMS: (ESI): RT=1.595 min, m/z calcd. for C₁₀₆H₁₄₇FN₂₄O₃₁, 1135.53 [M+2H]²⁺, m/z found 1136.0 [M+2H]²⁺; Mobile Phase: 1.5 ML/4 L TFA in water (solvent A) and 0.75 ML/4 L TFA in acetonitrile (solvent B),using the elution gradient 10%-80% (solvent B) over 2 minutes and holding at 80% for 0.48 minutes at a flow rate of 0.8 ml/min; Chromolith Flash RP-C18 2.1-30 mm

HPLC: RT=8.17 min, 98.18% purity. HPLC method A: Column: YMC-Pack ODS-A150*4.6 mm,5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min.

### 5.19 Preparation of (3S)-4-[[(1S)-1-[[4-[4-[4-[[2-[[2-[[2-[[2-[[(2S)-2-[[4-(153- azatricyclohexadeca-8(18),10(20),12(22),15(25),77(79),81(85)-hexaen-42(44)-yn-153-yl)-4-oxo-butanoyl]amino]-3-[6-[[4-(153-azatricyclohexadeca-8(18),10(20),12(22),15(25),77(79),81(85)-hexaen-42(44)-yn-153-yl)-4-oxo-butanoyl]oxymethyl]-3,4,5-trihydroxytetrahydropyran-2-yl]oxy-propanoyl]amino]acetyl]amino]acetyl]amino]acetyl]amino]acetyl]amino]butoxy]-2-ethyl-84-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxopropanoyl]amino]-3-(2H-tetrazol-5-yl)propanoyl]amino]acetyl]amino]butanoyl]amino]-2-methyl-propanoyl]amino]-3-hydroxybutanoyl]amino]-3-hydroxy-propanoyl]amino]-4-oxo-butanoic acid (LP20)

To a solution of 70 (7.90 mg, 18.54 μmol, 2.5 eq) was added DIPEA (4.79 mg, 37.07 μmol, 6.46 μL, 5 eq) in DMF (0.5 mL) at 25° C. The reaction was stirred at 25° C. for 16 h. Then H2O (2.5 mL) was added and it was stirred at 25° C. for 2 h. LCMS showed the reaction was complete. The reaction was filtered to give a residue as a yellow solution. The residue was purified by prep-HPLC (column: Phenomenex Gemini-NX 150*30 mm*5 μm; mobile phase: [water (0.04% NH₃H₂O+10 mM NH₄HCO₃)-ACN]; B %: 17%-47%, 8 min) to give LP20 (3.7 mg, 1.52 μmol, 20.82% yield, 95.093% purity) as a white solid.

LCMS: (ESI): RT=1.328 min, m/z calcd. for C₁₁₁H₁₄₃FN₂₄O₃₀, 1155.52 [M+2H]²⁺, m/z found 1156.0 [M+2H]²⁺; Mobile Phase: 0.8 mL/4 L NH₃·H₂O in water (solvent A) and acetonitrile (solvent B),using the elution gradient 10%-80% (solvent B) over 2 minutes and holding at 80% for 0.48 minutes at a flow rate of 1 ml/min; Column: XBridge C18 3.5 μm 2.1*30 mm; Wavelength:UV 220 nm & 254 nm; Column temperature: 50° C.

HPLC: RT=1.328 min, 92.31% purity. HPLC method A: Mobile Phase: 0.8 mL/4 L NH3·H2O in water (solvent A) and acetonitrile (solvent B), using the elution gradient 0%-60% (solvent B) over 5 minutes and holding at 60% for 0.48 minutes at a flow rate of 1 ml/min; Column: Xbridge Shield RP18 5 μm 2.1*50 mm; Wavelength: UV 220 nm & 254 nm.

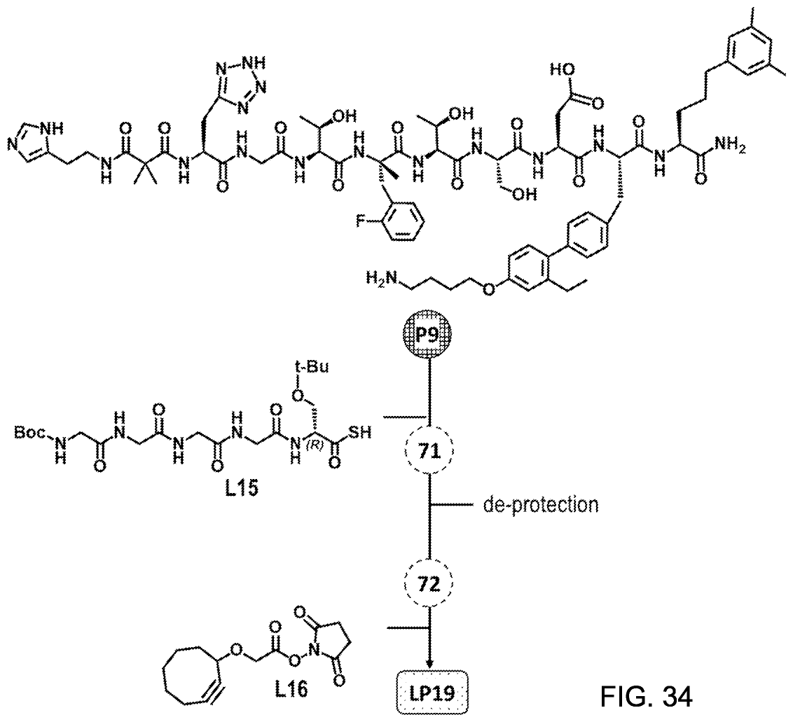

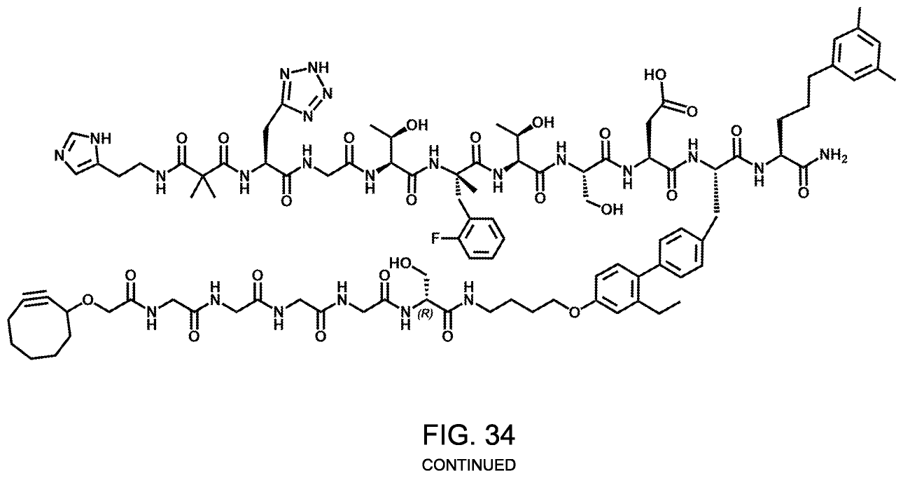

FIG. 34 depicts synthesis of linker-payload LP19 according to the disclosure.

### 5.20 Preparation of (3S)-4-[[(1S)-2-[[(1S)-1-carbamoyl-4-(3,5-dimethylphenyl)butyl]amino]-1-[[4-[4-[4-[[(2R)-2-[[2-[[2-[[2-[[2-[(2-cyclooct-2-yn-1-yloxyacetyl)amino]acetyl]amino]acetyl]aminol acetyl]amino]acetyl]amino]-3-hydroxypropanoyl]amino]butoxy]-2-ethyl-phenyl]phenyl]methyl]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxypropanoyl]amino]-4-oxo-butanoic acid (LP19)

To a colorless solution of P9 (25 mg, 16.17 μmol, 1 eq), L15 (16.35 mg, 32.35 μmol, 2.0 eq) and HOBt (4.37 mg, 32.35 μmol, 2.0 eq), DIPEA (3.14 mg, 24.26 μmol, 4.23 μL, 1.5 eq) in DMF (0.3 mL) was added a solution of 12 (2.87 mg, 11.32 μmol, 2.28 μL, 0.7 eq) in DMF (0.25 mL). The solution was stirred at 20° C. for 1 hr. LCMS trace showed the reaction was converted completely and the desired product was observed. The solution was treated with EtOAc (25 mL), the formed precipitate was collected by centrifuged for 5 min (5000 R). The collected white solid was lyophilized in freeze dryer to give compound 71 (45 mg, 21.11 μmol, 93.25% yield, 94.65% purity) as a white solid.

LCMS (ESI): RT=0.880 min, m/z calcd. for C₉₅H₁₂₆FN₂₃O₂₃ [M−Boc+2H]²⁺958.97, found 959.0. LC-MS method A: a Xtimate C18 2.1*30 mm, 3 μm column, with a flow rate of 1.2 mL/min, eluting with a gradient of 5% to 95% acetonitrile containing 0.75 ML/4 L TFA (solvent B) and 1.5 ML/4 L TFA in water (solvent A).

HPLC: RT=4.43 min. Mobile phase: 1.0% ACN in water (0.1% TFA) to 5% ACN in water (0.1% TFA) in 1 min; then from 5% ACN in water (0.1% TFA) to 100% ACN (0.1% TFA) in 5 minutes; hold at 100% ACN (0.1% TFA) for 2 minutes; back to 1.0% ACN in water (0.1% TFA) at 8.01 min, and hold two minutes. Flow rate: 1.2 ml/min

To a solution of 71 (35 mg, 17.35 μmol, 1 eq) in DCM (1 mL) was added TFA (1.54 g, 13.51 mmol, 1 mL, 778.42 eq). The solution was stirred at 20° C. for 1 hr. LCMS trace showed the reaction was converted completely and the desired product was observed. The solution was concentrated in vacuum to give a residue. The residue was purified by reversed phase HPLC (0.4% AcOH) (20 g column, Eluent of 0˜44% CH₃CN/H₂O, gradient @ 25 mL/min). The desired fluent was lyophilized in freeze dryer to give 72 (18 mg, 8.91 μmol, 51.35% yield, 92.108% purity) as a white solid.

LCMS (ESI): RT=0.815 min, m/z calcd. for C₃₆H₁₂₀FN₂₃O₂₃ [M+2H]2+930.94, found 931.2. LC-MS method A: a Xtimate C18 2.1*30 mm, 3 μm column, with a flow rate of 1.2 mL/min, eluting with a gradient of 5% to 95% acetonitrile containing 0.75 ML/4 L TFA (solvent B) and 1.5 ML/4 L TFA in water (solvent A).

To a solution of 72 (17 mg, 9.13 μmol, 1 eq) and L16 (5.10 mg, 18.27 μmol, 2.0 eq) in DMF (0.5 mL) was added DIPEA (2.36 mg, 18.27 μmol, 3.18 μL, 2.0 eq). The solution was stirred at 20° C. for 1 hr. LCMS trace showed the reaction was converted completely and the desired product was observed. The solution was purified by prep-HPLC (FA condition: Column: Phenomenex Gemini-NX 150*30 mm*5 μm; mobile phase: [water (0.1% TFA)-ACN]; B %: 0%-50%, 35 min). The desired fluent was lyophilized in freeze dryer to give LP19 (8.36 mg, 4.12 μmol, 36.34% yield, 99.72% purity) as a white solid.

LCMS (ESI): RT=3.379 min, m/z calcd. for C₃₆H₁₃₂FN₂₃O₂₅ [M+2H]²⁺1012.98, found 1013.7. LCMS conditions: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate 3 μm, C18,2.1*30 mm;

HPLC: RT=8.70 min, Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: YMC-Pack ODS-A 150*4.6 mm, 5 μm.

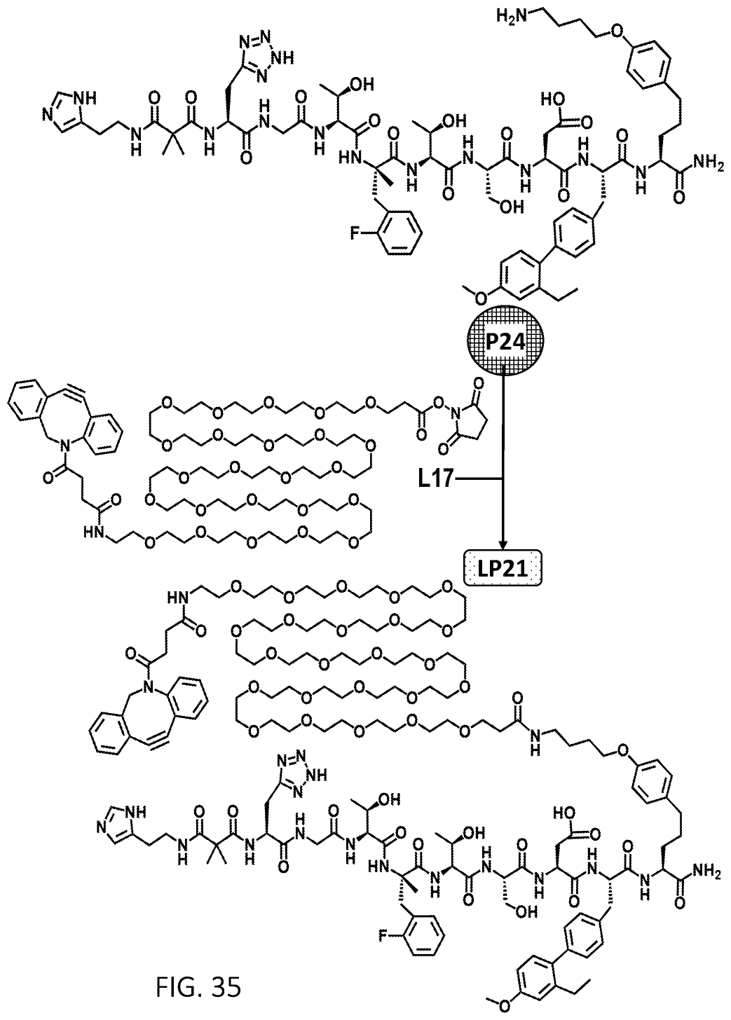

FIG. 35 depicts synthesis of linker-payload LP21 according to the disclosure.

### 5.21 Preparation of (3S)-4-[[(1S)-2-[[(1S)-4-[4-[4-[3-[2-[2-[2-[2-[2-[2-[2-[2-[2-[2-[2-[2-[2-[2-[2-[2-[2-[2-[2-[2-[2-[2-[2-[2-[[4-(163-azatricyclohexadeca-7(13),8(14),9(15),11(17),108,110(113)-hexaen-33-yn-163-yl)-4-oxo-butanoyl]amino]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxyl ethoxy]ethoxy]ethoxy]ethoxy]ethoxy]ethoxy]propanoylamino]butoxy]phenyl]-1-carbamoyl-butyl]amino]-1-[[4-(2-ethyl-4-methoxy-phenyl)phenyl]methyl]-2-oxo-ethyl]amino]-3-[[(2S)-2-[[(2R,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP21)

To a mixture of P24 (15 mg, 9.69 μmol, 1 eq.) and L17 (22.25 mg, 14.54 μmol, 1.5 eq.) in DMF (1 mL) was added DIPEA (6.26 mg, 48.46 μmol, 8.44 μL, 5 eq.) in one portion at 25° C. under nitrogen. The mixture was stirred at 25° C. for 2 h. LCMS trace showed that the reaction was converted completely. The reaction mixture was concentrated in vacuum to give residue. The residue was purified by prep-HPLC (column: mobile phase: [water(10 mM NH4HCO3)-ACN]; B %: 20%-50%, 55 min) to give pure product. The product was suspended in water (10 mL), the mixture frozen in a dry-ice/ethanol bath, and then lyophilized to dryness to afford the desired product LP21 (8.2 mg, 2.70 μmol, 27.86% yield, 97.5798% purity) as a white solid.

LCMS (ESI): RT=0.924 min, mass calcd. for C₁₄₄H₂₁₃FN₂₀O₄₅ 2961.50 [M+H]⁺, 987.167 [M+3H]³⁺, m/z found 988.8 [M+3H]³⁺. Reverse phase LCMS was carried out using a Chromolith Flash RP-18e 25-3 mm column, with a flow rate of 1.5 mL/min, eluting with a gradient of 5% to 95% acetonitrile containing 0.04% TFA (solvent B) and water containing 0.06% TFA (solvent A).

HPLC condition: RT=14.496 min, Reverse phase HPLC was carried out using a Gemini-NX 5u C18 110A 150*4.6 mm column, with a flow rate of 1.0 mL/min, eluting with a gradient of 10% to 80% acetonitrile containing 0.1% TFA (solvent B) and water containing 0.1% TFA (solvent A).

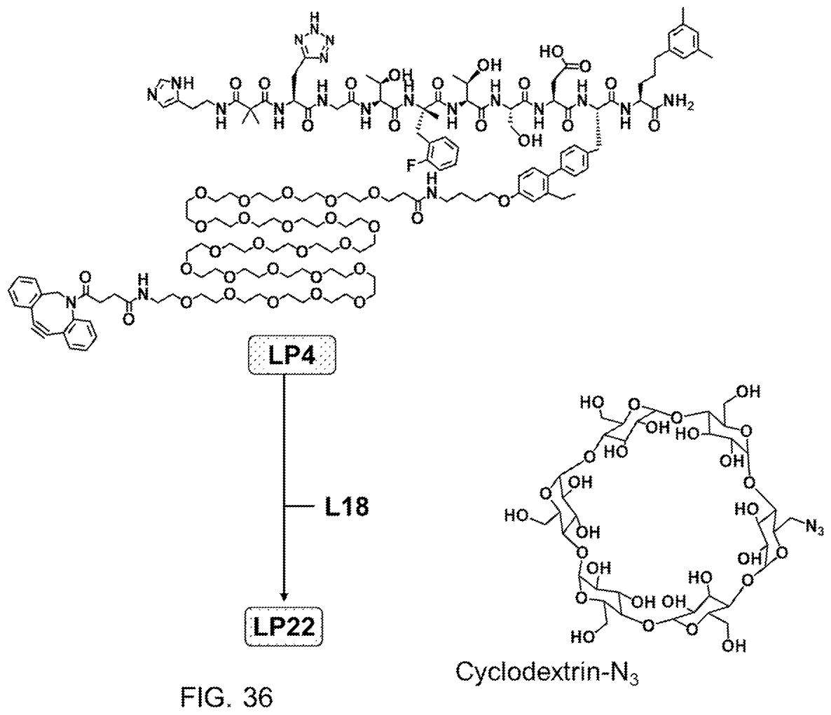

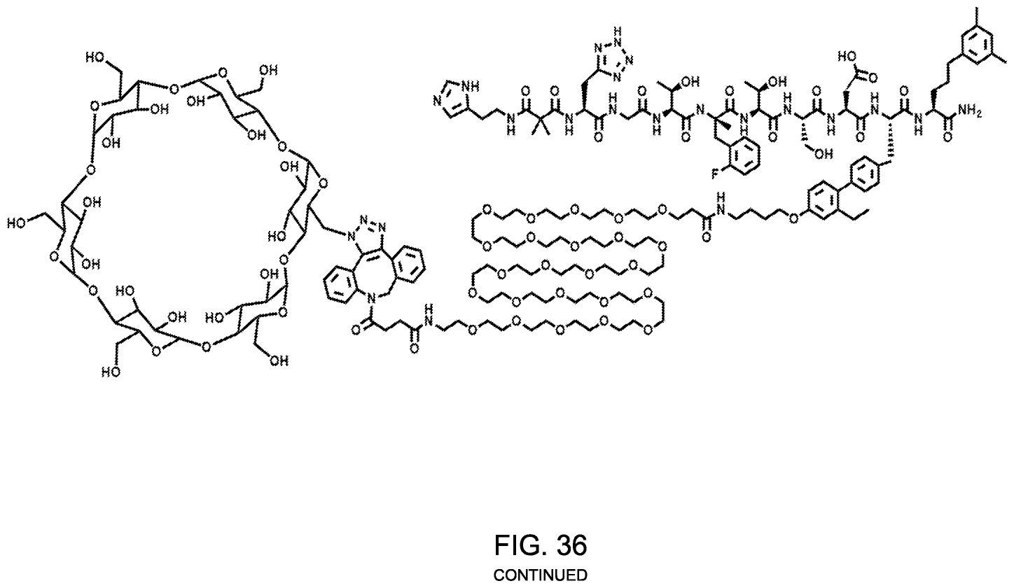

FIG. 36 depicts synthesis of linker-payload LP22 according to the disclosure.

### 5.22 Preparation of (3S)-4-[[(1S)-2-[[(1S)-1-carbamoyl-4-(3,5-dimethylphenyl)butyl]amino]-1-[[4-[4-[4-[3-[2-[2-[2-[2-[2-[2-[2-[2-[2-[2-[2-[2-[2-[2-[2-[2-[2-[2-[2- [2-[2-[2-[2-[2-[[4-[202-[[131,132,133, 134,135,136,137,138,139,140,141,142-dodecahydroxy-126,127,128,129,130-pentakis (hydroxymethyl)-240,241,242,243,244,245,246,247,248,249,250,251-dodecaoxahepta cyclodotetracontan-125-yl]methyl]-187,189,202,203-tetrazatetracyclononadeca-8(14),10(16),11(17),12(18),112(114),115(117),123,187(189)-octaen-203-yl]-4-oxo-butanoyl]amino]ethoxy]ethoxy]ethoxy]ethoxy]ethoxy]ethoxy]ethoxy]ethoxyl ethoxy]ethoxy]ethoxy]ethoxy]ethoxy]ethoxy]ethoxy]ethoxy]ethoxy]ethoxy]ethoxy]ethoxy]ethoxy]eth oxy]ethoxy]ethoxy]propanoylamino]butoxy]-2-ethyl-116-phenyl]phenyl]methyl]-2-oxo-ethyl]amino]-3-[[(2S)-2-[[(2R,3R)-2-[[(2S)-3-(2-fluorophenyl)-2-[[(2S,3R)-3-hydrox-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP22)

To a mixture of compound LP4 (5 mg, 1.69 μmol, 1 eq.) in DMF (0.3 mL) was added and compound L18 (cyclodextrin-N₃, 4.38 mg, 4.39 μmol, 2.6 eq.) in one portion at 25° C. under N2. The mixture was stirred at 25° C. for 12 hours. LCMS and HPLC trace showed the reaction were complete. The residue was purified by prep-HPLC (neutral: column: Waters Xbridge 150*25 5u; mobile phase: [water (10 mM NH₄HCO₃)-ACN]; B %: 20%-50%, 7 min) to give LP22 (1.66 mg, 4.03e-1 μmol, 23.88% yield, 96.149% purity) as a white solid.

HPLC: RT=11.976 min, Reverse phase HPLC was carried out using a Gemini-NX 5u C18 110A 150*4.6 mm column, with a flow rate of 1.0 mL/min, eluting with a gradient of 10% to 80% acetonitrile containing 0.1% TFA (solvent B) and water containing 0.1% TFA (solvent A).

LCMS (ESI): RT=0.877 min, mass calcd. for C₇₄H₁₂₀N₃O₃₀ 3956.84 [M+H]⁺, 1318.95 [M+3H]3+, found 1320.2 [M+3H]3+. Reverse phase LC-MS was carried out using a Chromolith Flash RP-18e 25-3 mm column, with a flow rate of 1.5 mL/min, eluting with a gradient of 5% to 95% acetonitrile containing 0.04% TFA (solvent B) and water containing 0.06% TFA (solvent A).

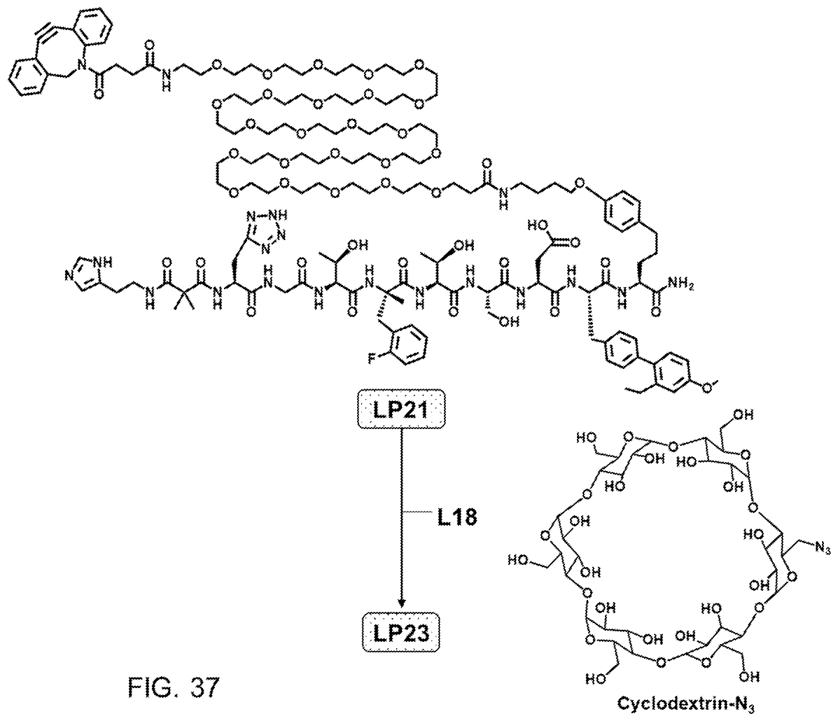

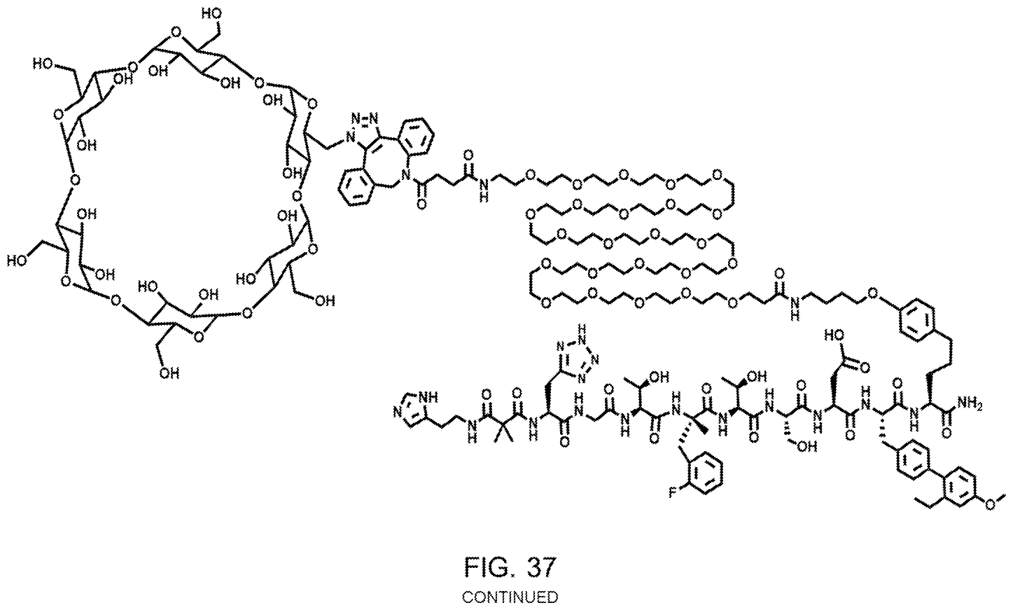

FIG. 37 depicts synthesis of linker-payload LP23 according to the disclosure.

### 5.23 Preparation of (3S)-4-[[(1S)-2-[[(1S)-1-carbamoyl-4-[4-[4-[3-[2-[2-[2-[2-[2-[2-[2-[2-[2-[2-[2-[2-[2-[2-[2-[2-[2-[2-[2-[2-[2-[2-[2-[2-[[4-[201-[[130,131,132,133,134,135,136,137,138,139,140, 141-dodecahydroxy-125,126,127,128,129-pentakis(hydroxymethyl)-239,240,241,242,243, 244, 245,246,247,248,249,250-dodecaoxaheptacyclodotetracontan-124-yl]methyl]-186,188,201, 202-tetrazatetracyclononadeca-7(13),9(15),10(16),11(17), 112(114),115(117),122, 186 (188)-octaen-202-yl]-4-oxo-butanoyl]amino]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]propanoylamino]butoxy]phenyl]butyl]amino]-1-[[4-(2-ethyl-4-methoxy-phenyl)phenyl]methyl]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP23)

Starting from LP21 (9.3 mg, 3.10 μmol, 1 eq.) and L18 (cyclodextrin-N₃, 3.7 mg, 3.7 μmol, 1.2 eq.) LP23 (3.5 mg, 8.84e-1 μmol, 50.00% yield, 100% purity) was obtained as a white solid following the same procedure in Example 22.

HPLC condition: RT=8.14 min, Reverse phase HPLC was carried out using a YMC-Pack ODS-A 150*4.6 mm, 5 μm, with a flow rate of 1.5 mL/min, eluting with a gradient of 10% to 80% acetonitrile containing 0.1% TFA (solvent B) and water containing 0.1% TFA (solvent A).

LCMS (ESI): RT=0.868 min, mass calcd. for C₇₅H₁₀₂FN₁₈O₁₇ 3958.82 [M+H]⁺ 1319.61 [M+H]3+, found 1321.5 [M+H]3+. Reverse phase LC-MS was carried out using a Chromolith Flash RP-18e 25-3 mm column, with a flow rate of 1.5 mL/min, eluting with a gradient of 5% to 95% acetonitrile containing 0.04% TFA (solvent B) and water containing 0.06% TFA (solvent A).

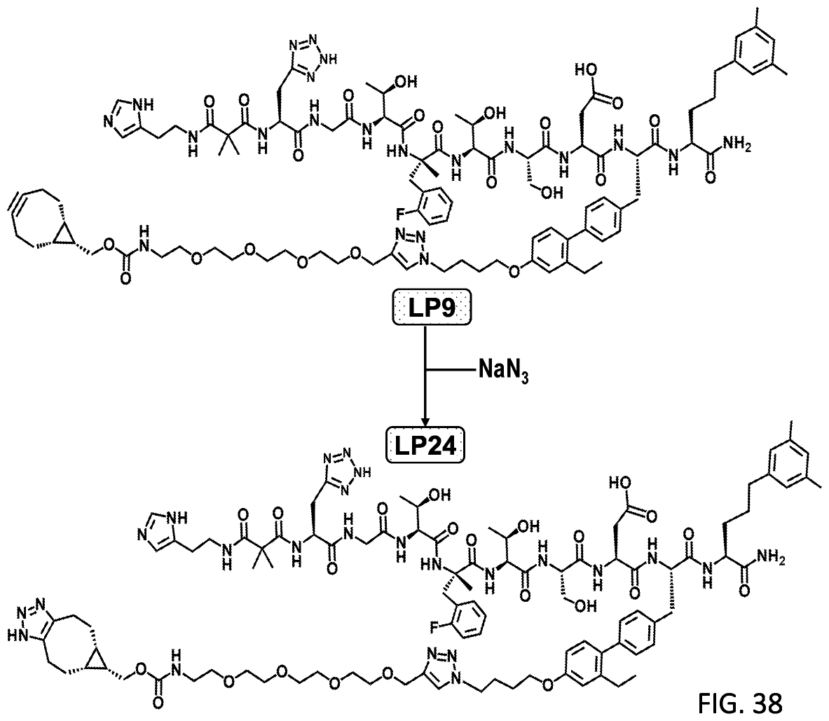

FIG. 38 depicts synthesis of linker-payload LP24 according to the disclosure.

### 5.24 Preparation of (3S)-4-[[(1S)-2-[[(1S)-1-carbamoyl-4-(3,5-dimethylphenyl)butyl]amino]-1-[[4-[2-ethyl-4-[4-[4-[2-[2-[2-[2-[[(68S,69R,71S)-106,107,108-triazatricyclododeca-72(107),106(108)-dien-71-yl]methoxycarbonylamino]ethoxy]ethoxy]ethoxy]ethoxymethyl]triazol-120-yl]butoxy]-61-phenyl]phenyl]methyl]-2-oxoethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP24)

A mixture of LP9 (1.13 mg, 0.571 μmol, 1 eq.) and NaN₃ (44.54 μg, 0.685 μmol, 1.2 eq.) in DMSO (0.1 mL) was stirred at 37° C. for 16 h. LCMS showed the reaction was complete. The crude product LP24 (1.15 mg, 0.569 μmol, 100.00% yield) was sent for the bio-assay directly without any further purification.

LCMS: (ESI): RT=3.561 min, m/z calcd. for C₉₇H1₃₅FN₂₄02₃, 1011.51 [M+2H]²⁺, found 1012.0 [M+2H]²⁺; Reverse phase LC-MS was carried out using method B.

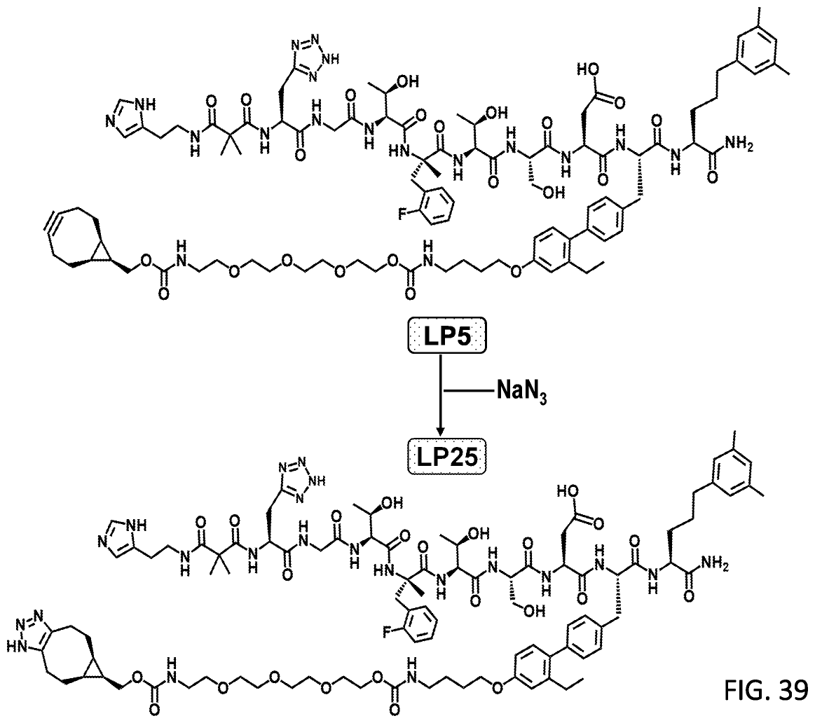

FIG. 39 depicts synthetic route of GLP-1R agonist Linker-payloads (LP25)

### 5.25 Preparation of (3S)-4-[[(1S)-2-[[(1S)-1-carbamoyl-4-(3,5-dimethylphenyl)butyl]amino]-1-[[4-[2-ethyl-4-[4-[2-[2-[2-[2-[[(65R,66S,68R)-102,103,104-triazatricyclododeca-69(103),102(104)-dien-68-yl]methoxycarbonylamino]ethoxy]ethoxy]ethoxy]ethoxycarbonylamino]butoxy]-59-phenyl]phenyl]methyl]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methylpropanoyl]amino]-3-hydroxy-butanoyl]amino]-3-hydroxy-propanoyl]amino]-4-oxo-butanoic acid (LP25)

A mixture of LP5 (1.12 mg, 0.577 μmol, 1 eq.) and NaN₃ (45.01 μg, 0.692 μmol, 1.2 eq.) in DMSO (0.1 mL) was stirred at 37° C. for 16 h. LCMS showed the reaction was complete. The crude product LP25 (1.14 mg, 4.42e-1 μmol, 76.68% yield, 77% purity) was sent for the bio-assay directly without any further purification.

LCMS: (ESI): RT=3.571 min, m/z calcd. for C₁₀₅H₁₃₄FN₂₇O₂₅, 992.49 [M+2H]²⁺, found 993.0 [M+2H]²⁺; Reverse phase LC-MS was carried out using method B.

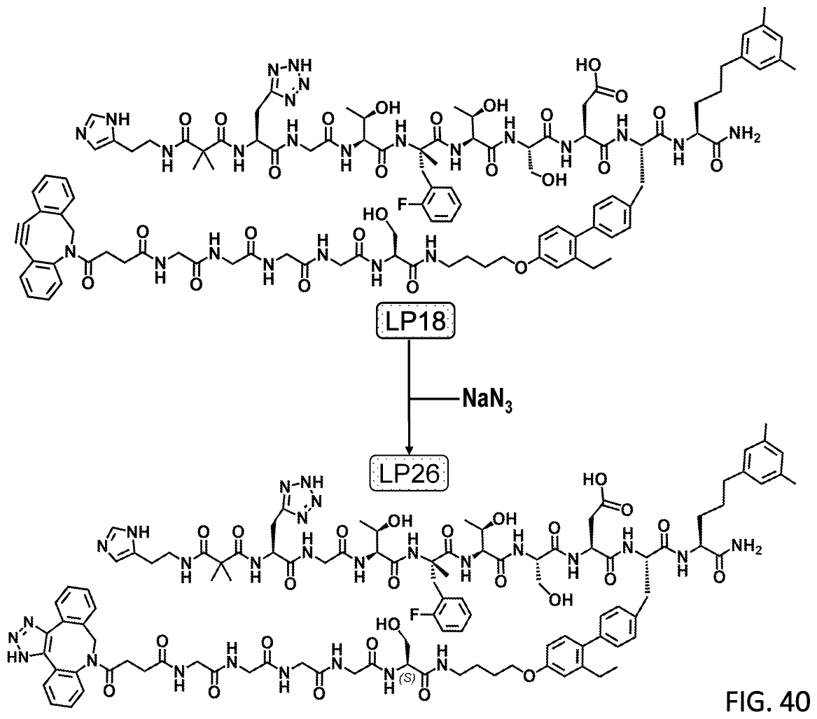

FIG. 40 depicts synthetic route of GLP-1R agonist Linker-payloads (LP26)

### 5.26 Preparation of (3S)-4-[[(1S)-2-[[(1S)-1-carbamoyl-4-(3,5-dimethylphenyl)butyl]amino]-1-[[4-[2-ethyl-4-[4-[[(2S)-3-hydroxy-2-[[2-[[2-[[2-[[2-[[4-oxo-4-(112,113,114,131-tetrazatetracyclononadeca-8(14),10(16),11(17),12(18),61(64),65,73(113),112(114)-octaen-131-yl)butanoyl]amino]acetyl]amino]acetyl]amino]acetyl]amino]acetyl]amino]propanoyl]amino]butoxy]-63-phenyl]phenyl]methyl]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP26)

A mixture of LP18 (1 mg, 4.65e-1 μmol, 1 eq.) and NaN₃ (36.31 μg, 0.559 μmol, 1.2 eq.) in DMSO (0.1 mL) was stirred at 37° C. for 16 h. LCMS showed the reaction was complete. The crude product LP26 (1.02 mg, 0.465 μmol, 100.00% yield) was sent for the bio-assay directly without any further purification.

LCMS: (ESI): RT=3.259 min, m/z calcd. for C₁₀₅H₁₃₄FN₂₇O₂₅, 1096.0, found 1096.7 [M+2H]²⁺; Reverse phase LC-MS was carried out using method F.

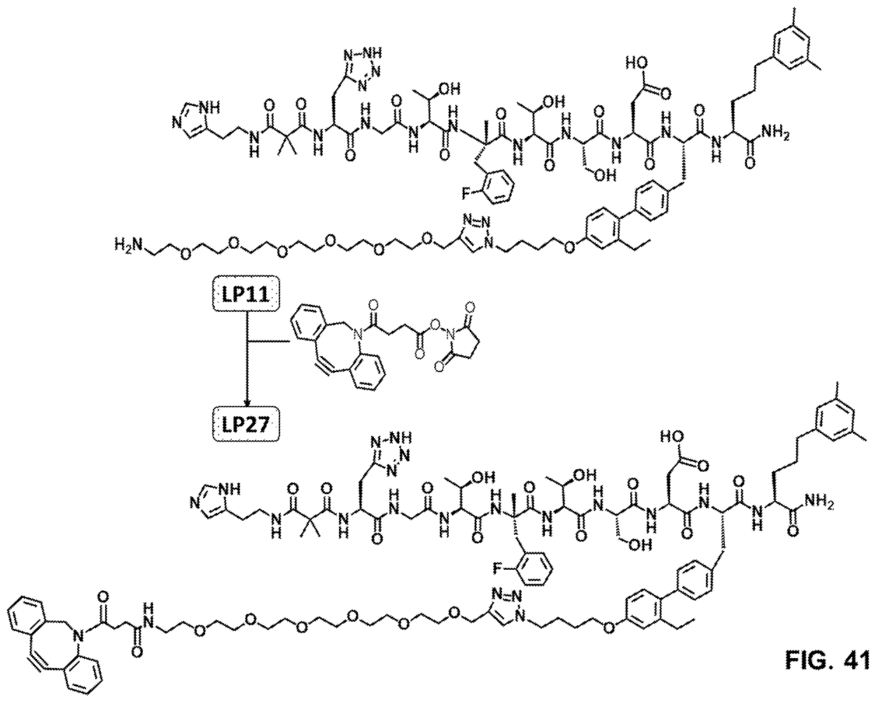

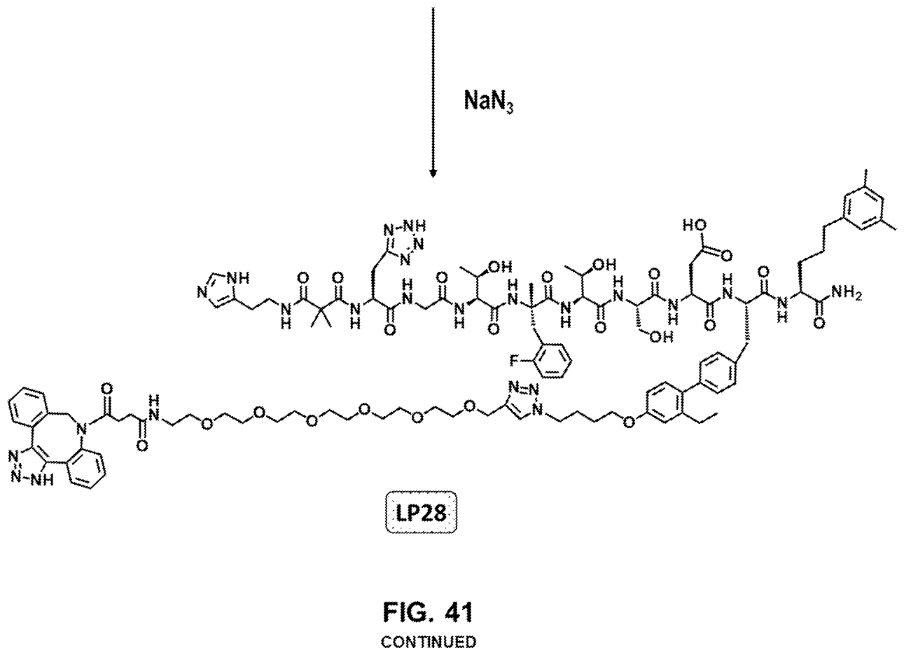

FIG. 41 depicts synthetic route of GLP-1R agonist Linker-payloads (LP27 and LP28)

### 5.27 Preparation of (3S)-4-[[(1S)-1-[[4-[4-[4-[4-[2-[2-[2-[2-[2-[2-[2-[2-[[4-(2-azatricyclo[10.4.0.04,91 hexadeca-1(12),4(9),5,7,13,15-hexaen-10-yn-2-yl)-4-oxo-butanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H- imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP27)

To a solution of LP11 (70 mg, 35.37 μmol, 1 eq) and DIBAC-suc-OSu (19.92 mg, 49.51 μmol, 1.4 eq) in DMF (0.5 mL) was added DIPEA (9.14 mg, 70.74 μmol, 12.32 μL, 2 eq). The mixture was stirred at 25° C. for 1 hr. LCMS showed the reaction was converted completely and the desired product was observed. The solution was filtered and purified by prep-HPLC (neutral condition. column: Phenomenex Gemini-NX 80*30 mm*3 μm; mobile phase: [water (10 mM NH4HCO3)-ACN]; B %: 25%-55%,9 min). The desired fluent was lyophilized in freeze dryer to give LP27 (24.87 mg, 10.56 μmol, 29.86% yield, 96.235% purity) as a white solid.

LCMS (ESI): RT=2.316 min, m/z calcd. for C₁₁₃H₁₅₀FN₂₂O₂₇ 2266.09 [M+H]⁺, 756.03 [M+3H]³⁺, found 756.3 [M+3H]³⁺, LC-MS Conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

HPLC: RT=9.17 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: YMC-Pack ODS-A 150*4.6 mm, 5 μm.

### 5.28 Preparation of (3S)-4-[[(1S)-2-[[(1S)-1-carbamoyl-4-(3,5-dimethylphenyl)butyl]amino]-1-[[4-[2-ethyl-4-[4-[4-[2-[2-[2-[2-[2-[2-[2-[2-[[4-oxo-4-(3,4,5,13-tetrazatetracyclo[13.4.0.02,6.07,121nonadeca-1(15),2(6),3,7(12),8,10,16,18-octaen-13-VI) butanoyl]amino]ethoxy]ethoxy]ethoxy]ethoxy]ethoxy]ethoxy]ethoxy]ethoxymethyl]triazol-1-yl]butoxy]phenyl]phenyl]methyl]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP28)

To a solution of LP27 (3 mg, 1.32 μmol, 1 eq) in DMSO (0.5 mL) was added NaN₃ (258.14 ug, 3.97 μmol, 3 eq). The mixture was stirred at 25° C. for 1 hr. LC-MS showed LP27 was consumed completely and one main peak with desired mass was detected. The reaction was added NH₄Cl solution (5 mL). The aqueous layer was separated and extracted with EtOAc (5 mL*2). The organic layers were combined and washed with water/brine=1/1 (400 mL*2), dried over anhydrous Na₂SO₄, filtered and concentrated in vacuum to give product as brown oil. The residue was purified by prep-HPLC (TFA condition; column: Waters Xbridge BEH C18 100*25 mm*5 μm; mobile phase: [water(0.075% TFA)-ACN]; B %: 15%-55%,16 min) to give LP28 (1.02 mg, 4.23e-1 μmol, 31.99% yield, 95.869% purity) was obtained as a white solid.

LCMS (ESI): RT=3.512 min, m/z calcd. for C₁₁₃H₁₅₁FN₂₅O₂₇ 2309.11 [M+H]⁺, C₁₁₃H₁₅₂FN₂₅O₂₇ 1155.56 [M+2H]²⁺, found 1155.5 [M+2H]²⁺. LC-MS Conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

HPLC: RT=4.024 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: YMC-Pack ODS-A 150*4.6 mm, 5 μm.

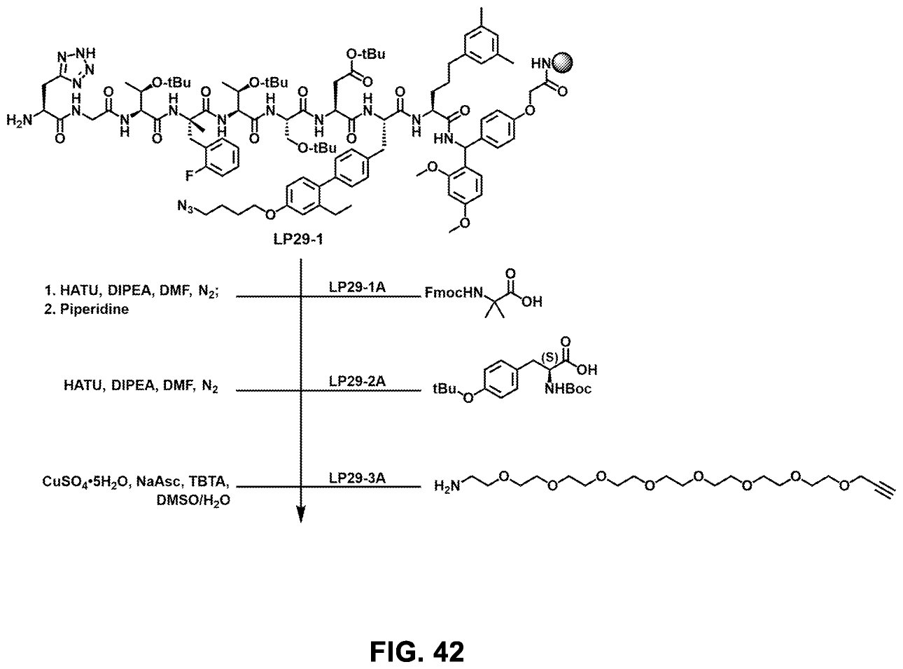

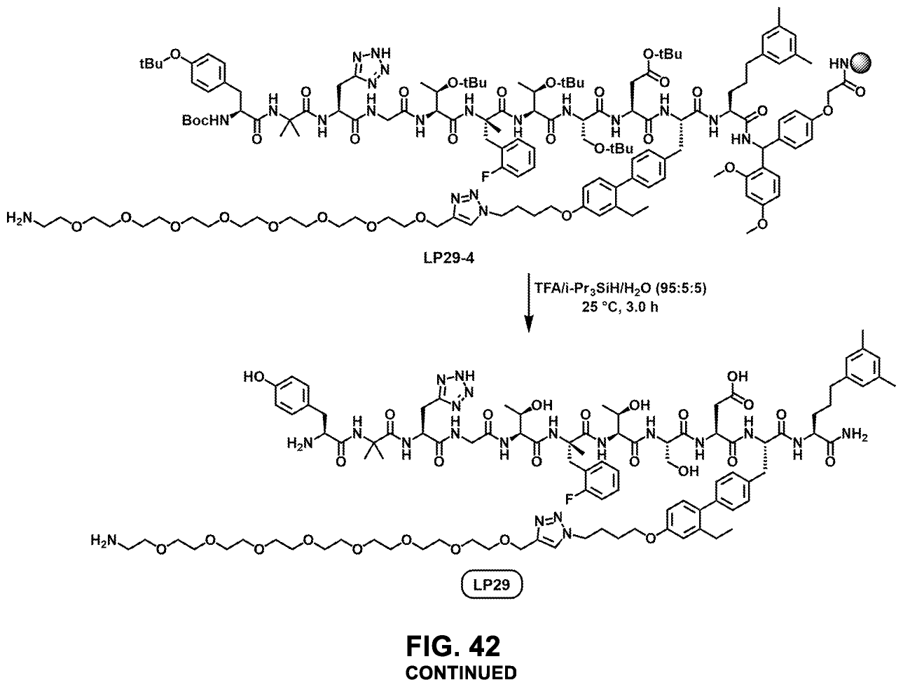

FIG. 42 depicts synthetic route of GLP-1R agonist Linker-payloads (LP29)

### Step 1: Preparation of tert-butyl (3S)-4-[[(1S)-2-[[(1S)-1-[[[4-(2-amino-2-oxo-ethoxy)phenyl]-(2,4-dimethoxyphenyl)methyl]carbamoyl]-4-(3,5-dimethylphenyl)butyl]amino]-1-[[4-[4-(4-azidobutoxy)-2-ethyl-phenyl]phenyl]methyl]-2-oxo-ethyl]amino]-3-[[(2S)-3-tert-butoxy-2-[[(2S,3R)-3-tert-butoxy-2-[[(2S)-2-[[(2S,3R)-3-tert-butoxy-2-[[2-[[(2S)-2-[[2-(9H-fluoren-9-ylmethoxycarbonylamino)-2-methyl-propanoyl]amino]-3-(2H-tetrazol-5-yl)propanoyl]amino]acetyl]amino]butanoyl]amino]-3-(2-fluorophenyl)-2-methyl-propanoyl]amino]butanoyl]amino]propanoyl]amino]-4-oxo-butanoate (LP29-2)

To a solution of LP29-1A (86.20 mg, 264.94 μmol, 1 eq) in DMF (10 mL) was added HATU (181.33 mg, 476.89 μmol, 1.8 eq) and DIPEA (136.96 mg, 1.06 mmol, 184.59 μL, 4 eq) at 25° C. over 10 min. Then the solution was added into LP29-1 (1 g, 264.94 μmol, 50% purity, 1 eq), and the mixture was bubbled with N₂ at 20° C. for 2 h. After completion, the mixture was filtered, and the collected resin was washed with DMF (30 mL*3), DCM (30 mL*3) to give the crude product on solid phase, which was swelled in 20% piperidine/DMF (10 mL), and the mixture was bubbled with N₂ at 25° C. for 2 hr. After completion, the mixture was filtered, and the collected resin was washed with DMF (100 mL*3), DCM (100 mL*3) to give the crude product bound on resin LP29-2 (264.94 μmol, crude) as a white solid.

LCMS (ESI): RT=3.923 min, m/z calcd. for C₁₀₂H₁₄₁FN₁₉O₂₀ 1448.70, found 1450.40 [M−4*tBu-C₁₇H₁₇NO₄+7H]⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

### Step 2: Synthesis of tert-butyl (3S)-4-[[(1S)-2-[[(1S)-1-[[[4-(2-amino-2-oxo-ethoxy)phenyl]-(2,4-dimethoxyphenyl)methyl]carbamoyl]-4-(3,5-dimethylphenyl)butyl]amino]-1-[[4-[4-(4-azidobutoxy)-2-ethyl-phenyl]phenyl]methyl]-2-oxo-ethyl]amino]-3-[[(2S)-3-tert-butoxy-2-[[(2S,3R)-3-tert-butoxy-2-[[(2S)-2-[[(2S,3R)-3-tert-butoxy-2-[[2-[[(2S)-2-[[2-[[(2S)-2-(tert-butoxycarbonylamino)-3-(4-tert-butoxyphenyl)propanoyl]amino]-2-methyl-propanoyl]amino]-3-(2H-tetrazol-5-yl)propanoyl]amino]acetyl]amino]butanoyl]amino]-3-(2-fluorophenyl)-2-methyl-propanoyl]amino]butanoyl]amino]propanoyl]amino]-4-oxo-butanoate (LP29-3)

To a solution of LP29-2A (222.39 mg, 659.12 μmol, 5 eq) in DMF (5 mL) was added HATU (90.22 mg, 237.28 μmol, 1.8 eq) and DIPEA (68.15 mg, 527.30 μmol, 91.85 μL, 4 eq) at 25° over 10 min. Then the solution was added into LP29-2 (131.82 μmol, 1 eq), and the mixture was bubbled with N₂ at 25° C. for 1 h. After completion, the mixture was filtered, and the collected resin was washed with DMF (50 mL*3), DCM (50 mL*3) to give the crude product bound on resin LP29-3 (131.82 μmol, crude) as a yellow solid.

LCMS (ESI): RT=3.990 min, m/z calcd. for C₇₈H₁₀₂FN₁₉O₁₈ 806.88, found 807.3 [M−5tBu-Boc-C₁₇H₁₇NO₄]²⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

### Step 3: Synthesis of tert-butyl (3S)-4-[[(1S)-1-[[4-[4-[4-[4-[2-[2-[2-[2-[2-[2-[2-(2-aminoethoxy)ethoxy]ethoxy]ethoxy]ethoxy]ethoxy]ethoxy]ethoxymethyl]triazol-1-yl]butoxy]-2-ethyl-phenyl]phenyl]methyl]-2-[[(1S)-1-[[[4-(2-amino-2-oxo-ethoxy)phenyl]-(2,4-dimethoxyphenyl)methyl]carbamoyl]-4-(3,5-dimethylphenyl)butyl]amino]-2-oxo-ethyl]amino]-3-[[(2S)-3-tert-butoxy-2-[[(2S,3R)-3-tert-butoxy-2-[[(2S)-2-[[(2S,3R)-3-tert-butoxy-2-[[2-[[(2S)-2-[[2- [[(2S)-2-(tert-butoxycarbonylamino)-3-(4-tert-butoxyphenyl)propanoyl]amino]-2-methyl-propanoyl]amino]-3-(2H-tetrazol-5-yl)propanoyl]amino]acetyl]amino]butanoyl]amino]-3-(2-fluorophenyl)-2-methyl-propanoyl]amino]butanoyl]amino]propanoyl]amino]-4-oxo-butanoate (LP29-4)

To a solution of LP29-3 (22.23 μmol, 1 eq) and LP29-3A (17.78 mg, 43.64 μmol, 2 eq) in DMF (5 mL) was added SODIUM ASCORBATE (21.61 mg, 109.09 μmol, 5 eq), TBTA (11.58 mg, 21.82 μmol, 1 eq) and CuI (20.78 mg, 109.09 μmol, 5 eq). The mixture was stirred at 25° C. for 2 hr. After completion, the mixture was filtered, and the collected resin was washed with DMF (50 mL*3), DCM (50 mL*3) to give the crude product bound on resin LP29-4 (22.23 μmol, crude) as a green solid.

LCMS (ESI): RT=3.085 min, m/z calcd. for C₉₇H₁₃₉FN₂₀O₂₆ 1010.51, found 1011.0 [M+2H]²⁺, LC-MS Conditions: Mobile Phase: 1.5 ML/4 L TFA in water (solvent A) and 0.75 ML/4 L TFA in acetonitrile (solvent B), using the elution gradient 5%-95% (solvent B) over0.7 minutes and holding at 95% for 0.4 minutes at a flow rate of 1.5 mL/min; Column: Agilent Pursult 5 C18 20*2.0 mm.

### Step 4: Synthesis of (3S)-4-[[(1S)-1-[[4-[4-[4-[4-[2-[2-[2-[2-[2-[2-[2-(2-aminoethoxy)ethoxyl ethoxy]ethoxy]ethoxy]ethoxy]ethoxy]ethoxymethyl]triazo-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S, 3R)-2-[[(2S)-2-[[(2S, 3R)-2-[[2-[[(2S)-2-[[2-[[(2S)-2-amino-3-(4- hydroxyphenyl) propanoyl]amino]-2-methyl-propanoyl]amino]-3-(2H-tetrazol-5-yl)propanoyl]amino]acetyl]amino]-3-hydroxy-butanoyl]amino]-3-(2-fluorophenyl)-2-methyl-propanoyl]amino]-3-hydroxy-butanoyl]amino]-3-hydroxy-propanoyl]amino]-4-oxo-butanoic acid (LP29)

The resin bound compound LP29-4 (22.23 μmol, 1 eq) was subjected to acidic cleavage by using a TFA cocktail (TFA/TIPS/H₂O=95:2.5:2.5, 10 mL), the mixture was shaken at 25° C. for 2 hours. The mixture was filtered and the filtrate was diluted with t-BuOMe (100 mL) at 0° C. to give a precipitate, which was centrifuged (5000 R) for 10 min. The residue was purified by prep-HPLC (column: Boston Green ODS 150*30 mm*5 μm; mobile phase: [water(0.1% TFA)-ACN]; B %: 17%-57%,9 min) to give the product LP29 (7.55 mg, 3.69 μmol, 16.58% yield, 98.63% purity) as a white solid.

LCMS (ESI): RT=3.133 min, m/z calcd. for C₉₇H₁₃₉FN₂₀O₂₆ 1010.51, found 1011.0 [M+2H]²⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvents A).

HPLC: RT=3.77 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: YMC-Pack ODS-A 150*4.6 mm, 5 μm.

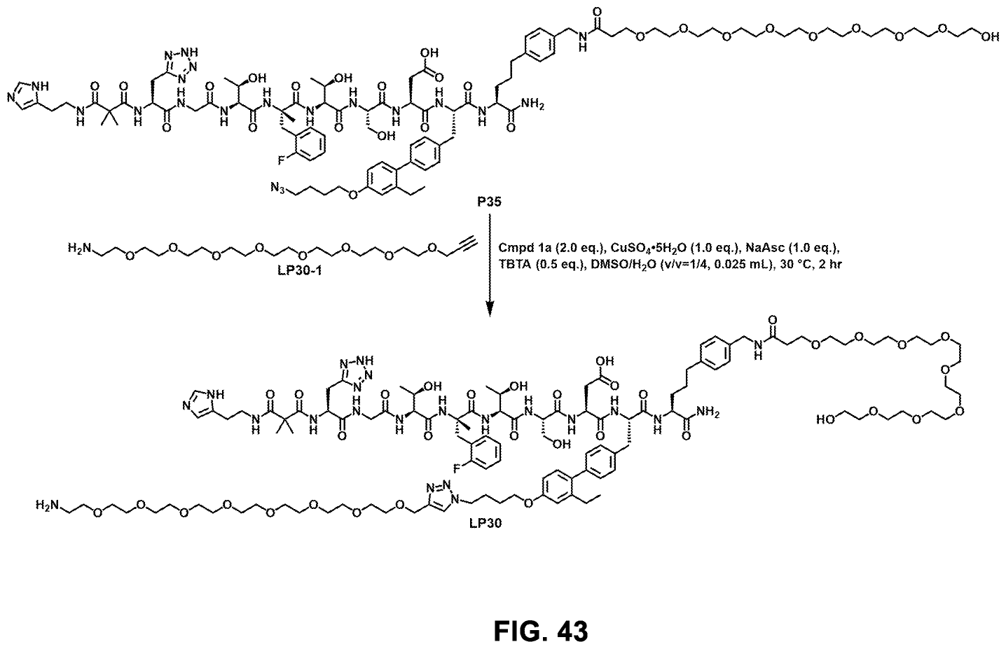

FIG. 43 depicts synthetic route of GLP-1R agonist Linker-payloads (LP30)

### 5.30 Preparation of (8S,14S,17S,20S,23S,26S)-8-((2H-tetrazol-5-yl)methyl)-26-(((S)-3-(4′-(4-(4-(25-amino-2,5,8,11,14,17,20,23-octaoxapentacosyl)-1H-1,2,3-triazol-1-yl)butoxy)-2′-ethyl-[1,1′-biphenyl]-4-yl)-1-(((S)-1-amino-5-(4-(23-hydroxy-3-oxo-6,9,12,15,18,21-hexaoxa-2-azatricosyl) phenyl)-1-oxopentan-2-yl)amino)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (LP30)

To a solution of P35 (15 mg, 7.51 μmol, 1 eq.) in H₂O (0.09 mL) was added a solution of CuSO₄·5H₂O (1.88 mg, 7.51 μmol, 1.0 eq.), sodium;(2R)-2-[(1S)-1,2-dihydroxyethyl]-4-hydroxy-5-oxo-2H-furan-3-olate (1.49 mg, 7.51 μmol, 1.0 eq.) in DMSO (0.03 mL), and followed by TBTA (1.99 mg, 3.76 μmol, 0.5 eq.) in H₂O (0.03 mL) and a solution of LP30-1 (6.12 mg, 15.02 μmol, 2.0 eq.) in DMSO (0.03 mL). The mixture was stirred at 30° C. for 2.5 hr. LCMS showed the desired MS was detected. The reaction mixture was purified by prep-HPLC (column: Waters X bridge BEH C18 100*25 mm*5 μm; mobile phase: [water (0.1% TFA)-ACN]; B %: 5%-42.5%, 12 min) to afford LP30 (2 mg, 8.15e-1 μmol, 10.85% yield, 98% purity) as a white solid.

LCMS (ESI): RT=2.628 min, mass calcd. for C₁₁₂H₁₇₄FN₂₂O₃₅ 2406.23 [M+H]⁺, 802.74 [M+3H]³⁺, found 802.20 [M+3H]³⁺. LC-MS Conditions: Mobile Phase:1.5 ML/4 L TFA in water (solvent A) and 0.75 ML/4 L TFA in acetonitrile (solvent B), using the elution gradient 10%-80% (solvent B) over 6.0 minutes and holding at 80% for 0.5 minutes at a flow rate of 0.8 ml/min; Column: Xtimate3 μm, C18,2.1*30 mm. Wave length: UV 220 nm&254 nm; Column temperature: 50° C.

HPLC: RT=6.32 min, 98% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

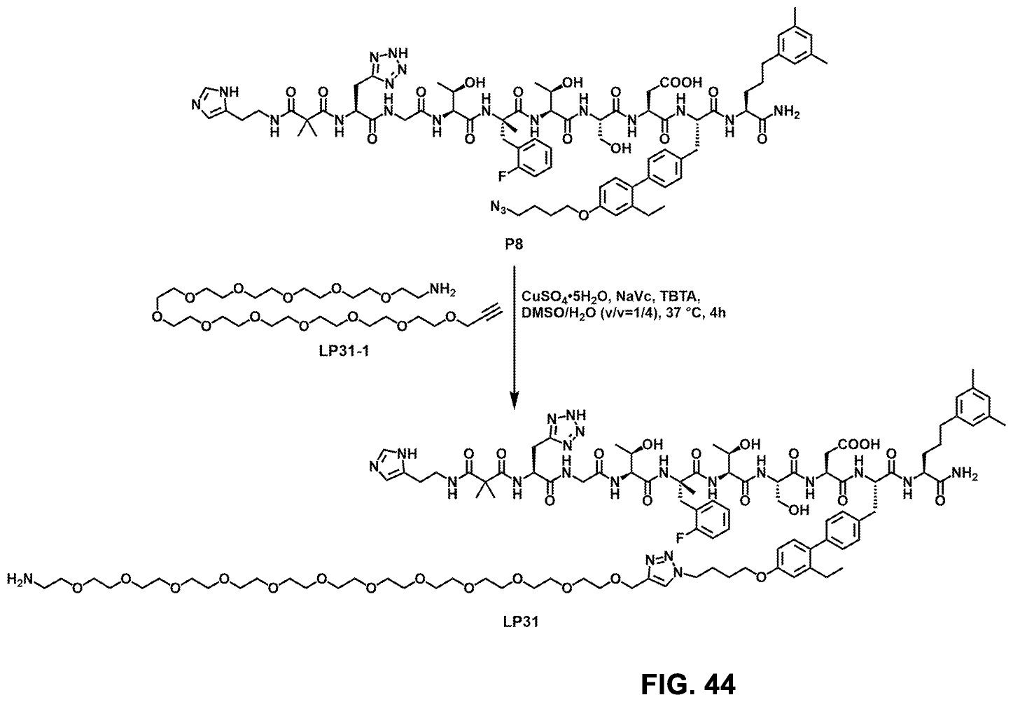

FIG. 44 depicts synthetic route of GLP-1R agonist Linker-payloads (LP31)

### 5.31 Preparation of (3S)-4-[[(1S)-1-[[4-[4-[4-[4-[2-[2-[2-[2-[2-[2-[2-[2-[2-[2-[2-(2-aminoethoxy) ethoxy]ethoxy]ethoxy]ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[[(1S)-1-carbamoyl-4-(3,5-dimethylphenyl) butyl]amino]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP31)

To a solution of P8 (40 mg, 25.45 μmol, 1 eq) and LP31-1 (29.71 mg, 50.90 μmol, 2 eq) in DMSO (0.1 mL) and H₂O (0.4 mL) was added TBTA (6.75 mg, 12.72 μmol, 0.5 eq), CuSO₄·5H₂O (6.35 mg, 25.45 μmol, 1 eq) and SODIUM ASCORBATE (5.04 mg, 25.45 μmol, 1 eq). The mixture was stirred at 37° C. for 4 hr. LC-MS showed the desired mass was detected. The green solution was filtered to give the crude product. The residue was purified by prep-HPLC (TFA condition. column: Welch Xtimate C18 100*40 mm*3 μm; mobile phase: [water (0.075% TFA)-ACN]; B %: 23%-53%, 10 min) to afford LP31 (batch 1: 11.59 mg, 5.21 μmol, 20.48% yield, 96.94% purity) and (batch 2: 11.13 mg, 4.95 μmol, 19.45% yield, 95.86% purity) as yellow gum.

Batch 1: LCMS (ESI): RT=3.160 min, m/z calcd. for C₁₀₂H₁₅₃FN₂₁O₂₉ 2155.10 [M+H]⁺, C₁₀₂H₁₅₄FN₂₁O₂₉ 1078.05 [M+2H]²⁺, found 1078.6 [M+2H]²⁺.LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=7.48 min. HPLC Conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: YMC-Pack ODS-A 150*4.6 mm, 5 μm.

Batch 2: LCMS (ESI): RT=3.147 min, m/z calcd. for C₁₀₂H₁₅₃FN₂₁O₂₉ 2155.10 [M+H]⁺, C₁₀₂H₁₅₄FN₂₁O₂₉ 1078.05 [M+2H]²⁺, found 1078.6 [M+2H]²⁺. LC-MS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

HPLC: RT=7.48 min. HPLC Conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: YMC-Pack ODS-A 150*4.6 mm, 5 μm.

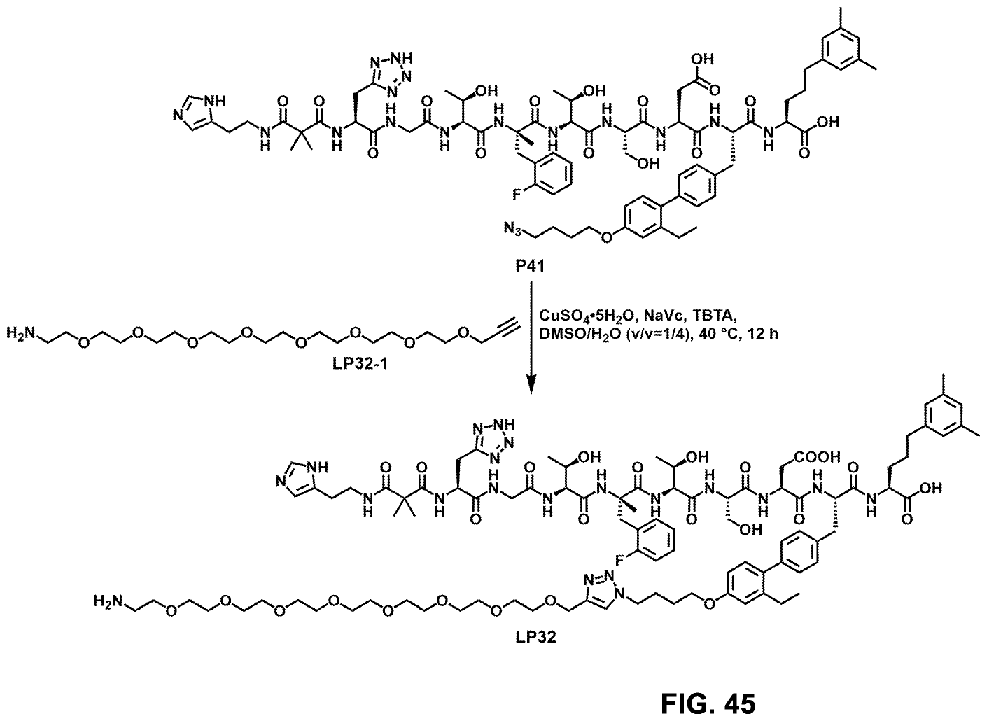

FIG. 45 depicts synthetic route of GLP-1R agonist Linker-payloads (LP32)

### 5.32 Preparation of (2S)-2-[[(2S)-3-[4-[4-[4-[4-[2-[2-[2-[2-[2-[2-[2-(2-aminoethoxy)ethoxy]ethoxy]ethoxy]ethoxy]ethoxy]ethoxy]ethoxymethyl]triazol-1-yl]butoxy]-2-ethyl-phenyl]phenyl]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]propanoyl]amino]-5-(3,5-dimethylphenyl)pentanoic acid (LP32)

To a solution of LP31-1 (35 mg, 22.25 μmol, 1 eq) in H₂O (1 mL) was added a solution of CuSO₄·5H₂O (5.56 mg, 22.25 μmol, 1 eq) and NaVc (4.41 mg, 22.25 μmol, 1 eq), a solution of TBTA (5.90 mg, 11.13 μmol, 0.5 eq) and a solution of P41 (18.14 mg, 44.51 μmol, 2 eq) in DMSO (0.25 mL). The mixture was stirred at 40° C. for 12 hr. The reaction mixture was diluted with H₂O (3 mL) and ACN (2 mL), then the mixture was filtered. The filtrate was purified by prep-HPLC (column: Welch Xtimate C18 100*40 mm*3 μm; mobile phase: [water (TFA)-ACN]; B %: 20%-60%,10 min) to give the LP31 (1 mg, 0.46 μmol, 10.11% yield, 91% purity).

LCMS (ESI): RT=2.910 min, m/z calcd. for C94H136FN20O26 991.05[M+H]⁺, found 990.9 [M+H]⁺.LC-MS method A: a MERCK, RP-18e 25-2 mm column, with a flow rate of 1.5 mL/min, eluting with a gradient of 10% to 80% acetonitrile containing 0.02% TFA (solvent B) and water containing 0.04% TFA (solvent A).

HPLC: RT=7.56 min; Mobile Phase: 2.75 ML/4 L TFA in water (solvent A) and 2.5 ML/4 L TFA in acetonitrile (solvent B), using the elution gradient 10%-80% (solvent B) over 10 minutes and holding at 100% for 5 minutes at a flow rate of 1.5 ml/min; Column: YMC-Pack ODS-A150*4.6 mm, 5 μm.

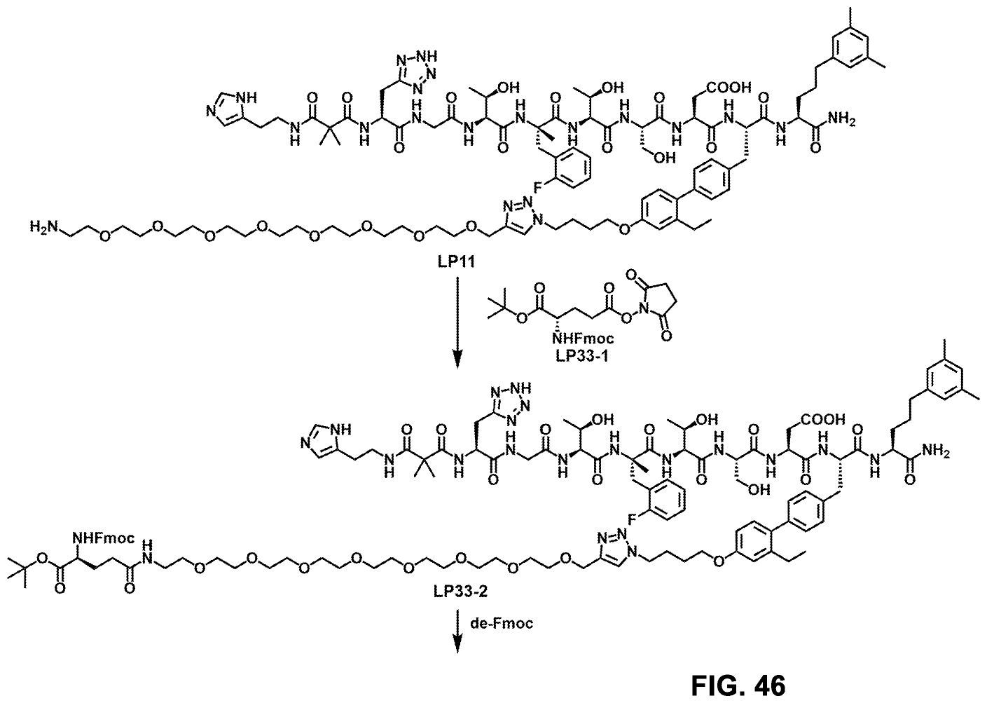

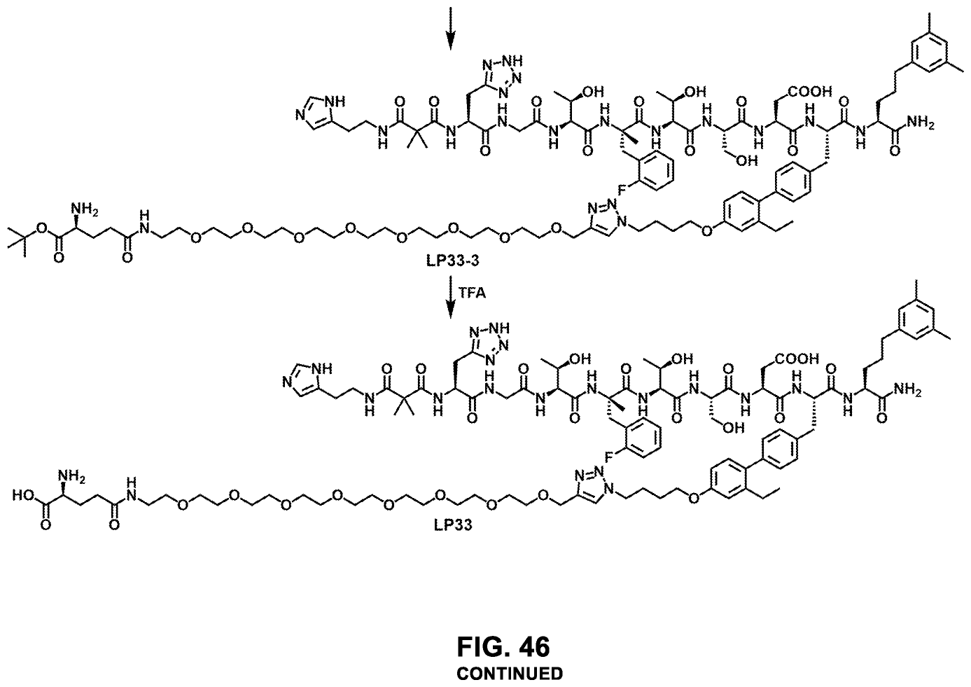

FIG. 46 depicts synthetic route of GLP-1R agonist Linker-payloads (LP33)

### Step 1: Synthesis of (3S)-4-[[(1S)-1-[[4-[4-[4-[4-[2-[2-[2-[2-[2-[2-[2-[2-[[(4S)-5-tert-butoxy-4-(9H-fluoren-9-ylmethoxycarbonylamino)-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[f(2S)-2-[[(2S,3R)-2-[[3-(2-fluorophenyl)-2-[[(2S,3R)-3-hydroxy-2-[[2-[[(2S)-2- [[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP33-2)

To a solution of LP11 (10 mg, 5.05 μmol, 1 eq.) and LP33-1 (2.9 mg, 5.5 μmol, 1 eq.) in DMF (2 mL) was added DIPEA (1.96 mg, 15.16 μmol, 2.64 μl, 3 eq.). The mixture was stirred at 20° C. for 12 hr. LC-MS showed LP11 was consumed completely and one main peak with desired mass was detected. LCMS (ESI): RT=4.030 min, m/z calcd. for C₁₁₈H₁₆₃O₃₀N₂₂F 796.6[M+3H]³⁺, found 796.5. Mobile Phase: 1.5 ML/4 L TFA in water (solvent A) and 0.75 ML/4 L TFA in acetonitrile (solvent B), using the elution gradient 10%-80% (solvent B) over 6 minutes and holding at 80% for 0.5 minutes at a flow rate of 0.8 ml/min; Column: Xtimate C18 2.1*30 mm, 3 μm; Wavelength: UV 220 nm, 254 nm; Column temperature: 50° C.; MS ionization: ESI. The reaction was purified by prep-HPLC (column: 0-phenomenex clarity RP 150*10 mm*5 μm; mobile phase: [water (0.075% TFA)-ACN]; B %: 20%-70%, 20 min) to give the product LP33-2 (8 mg, 3.12 μmol, 61.70% yield, 93% purity).

LCMS (ESI): RT=4.003 min, m/z calcd. for C118H163O30N22F 1194.17[M+2H]⁺/2, found 1194.3.Mobile Phase: 1.5 ML/4 L TFA in water (solvent A) and 0.75 ML/4 L TFA in acetonitrile (solvent B), using the elution gradient 10%-80% (solvent B) over 6 minutes and holding at 80% for 0.5 minutes at a flow rate of 0.8 ml/min; Column: Xtimate C18 2.1*30 mm, 3 μm; Wavelength: UV 220 nm, 254 nm; Column temperature: 50° C.; MS ionization: ESI.

### Step 2: (3S)-4-[[(1S)-1-[[4-[4-[4-[4-[2-[2-[2-[2-[2-[2-[2-[2-[[(4S)-4-amino- 5-tert-butoxy-5-oxopentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S, 3R)-2-[[3-(2-fluorophenyl)-2-[[(2S, 3R)-3-hydroxy-2-[[2-[[(2S)-2- [[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP33-3)

To a solution of LP32-2 (8 mg, 3.35 μmol, 1 eq.) in THF (0.5 mL) was added N-ethylethanamine (2.45 mg, 33.52 μmol, 3.45 μl, 10 eq.). The mixture was stirred at 20° C. for 3 hr. LC-MS showed LP32-3 was consumed completely and one main peak with desired mass was detected. LCMS (ESI): RT=3.070 min, m/z calcd. for C₁₁₈H₁₅₃O₂₈N₂₂F 1082.5[M+2H]²⁺, found 1082.9. Mobile Phase: 1.5 ML/4 L TFA in water (solvent A) and 0.75 ML/4 L TFA in acetonitrile (solvent B), using the elution gradient 10%-80% (solvent B) over 6 minutes and holding at 80% for 0.5 minutes at a flow rate of 0.8 ml/min; Column: Xtimate C18 2.1*30 mm, 3 μm; Wavelength: UV 220 nm, 254 nm; Column temperature: 50° C.; MS ionization: ESI. The reaction mixture was concentrated under reduced pressure to give LP33-3 (7 mg, crude) as a colourless oil.

### Step 3: (2S)-2-amino-5-[2-[2-[2-[2-[2-[2-[2-[2-[[1-[4-[4-[4-[(2S)-3-[[(1S)-1-carbamoyl-4-(3,5-dimethylphenyl)butyl]amino]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]butyl]triazol-4-yl]methoxy]ethoxy]ethoxy]ethoxy]ethoxy]ethoxy]ethoxy]ethoxy]ethylamino]-5-oxo-pentanoic acid (LP33)

To a solution of LP33-3 (7 mg, 3.23 μmol, 1 eq.) in DCM (0.5 mL) was added TFA (770.00 mg, 6.75 mmol, 500.00 μl, 2088.06 eq.). The mixture was stirred at 20° C. for 1 hr. LC-MS showed LP32-4 was consumed completely and one main peak with desired mass was detected. LCMS (ESI): RT=2.977 min, m/z calcd. for C₉₉H₁₄₅O₂₈N₂₂F 1054.52[M+2H]²⁺, found 1054.9. Mobile Phase: 1.5 ML/4 L TFA in water (solvent A) and 0.75 ML/4 L TFA in acetonitrile (solvent B), using the elution gradient 10%-80% (solvent B) over 6 minutes and holding at 80% for 0.5 minutes at a flow rate of 0.8 ml/min; Column: Xtimate C18 2.1*30 mm, 3 μm; Wavelength: UV 220 nm, 254 nm; Column temperature: 50° C.; MS ionization: ESI. The reaction mixture was filtered to give a residue. The residue was purified by prep-HPLC (column: O-phenomenex clarity RP 150*10 mm*5 μm; mobile phase: [water (0.075% TFA)-ACN]; B %: 15%-65%, 25 min) give the product LP33 (2.1 mg, 9.66e-1 μmol, 29.87% yield, 97% purity) as a white solid.

LCMS (ESI): RT=2.950 min, m/z calcd. for C₉₉H₁₄₅O₂₈N₂₂F 1054.52[M+2H]+/2, found 1054.9.Mobile Phase: 1.5 ML/4 L TFA in water (solvent A) and 0.75 ML/4 L TFA in acetonitrile (solvent B), using the elution gradient 10%-80% (solvent B) over 6 minutes and holding at 80% for 0.5 minutes at a flow rate of 0.8 ml/min; Column: Xtimate C18 2.1*30 mm, 3 μm; Wavelength: UV 220 nm, 254 nm; Column temperature: 50° C.; MS ionization: ESI.

UPLC RT=3.277 min Mobile Phase: 0.05% TFA in water (solvent A) and 0.05% TFA in acetonitrile (solvent B), using the elution gradient 5%-95% (solvent B) over 5 minutes, later holding at 95% for 3 minutes at a flow rate of 0.6 ml/minutes; Column: Waters ACQUITY UPLC HSS T3 1.8 um, 2.1×100 mm.

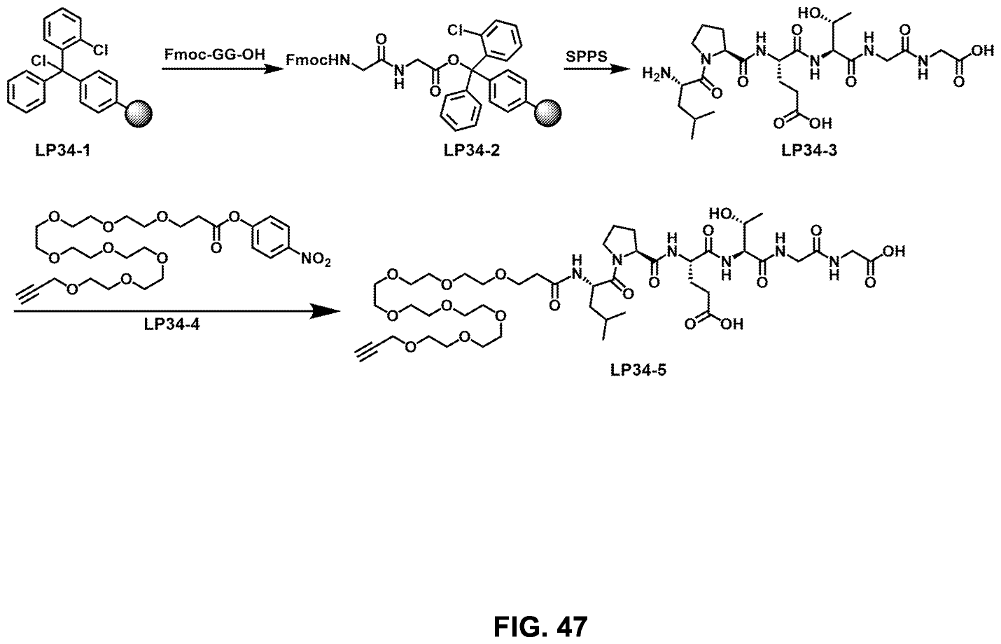

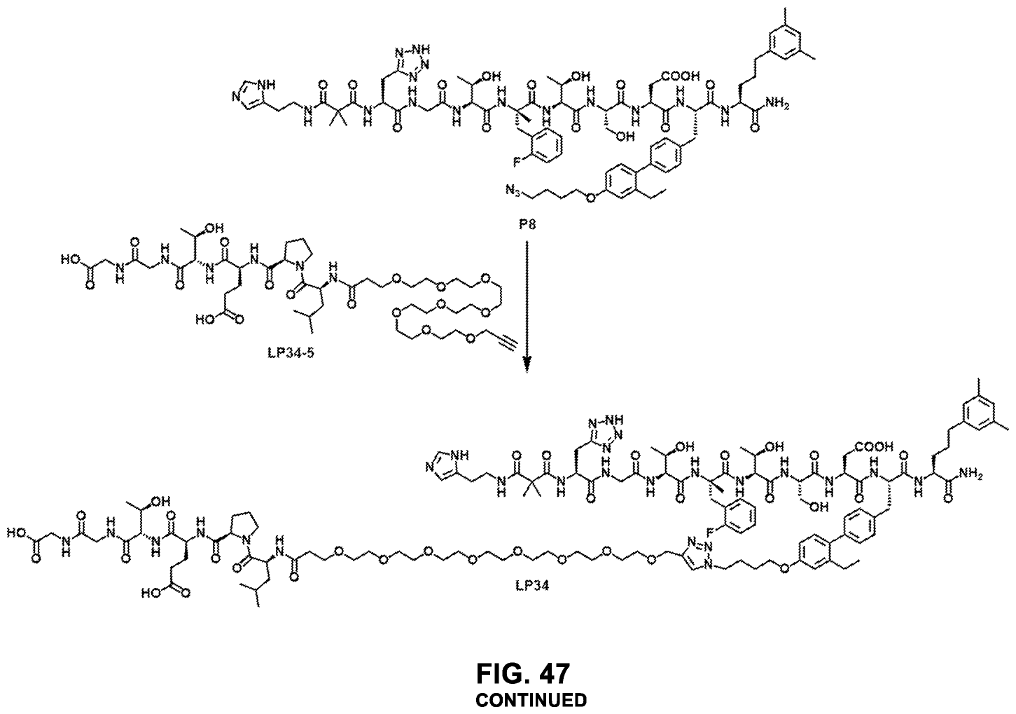

FIG. 47 depicts synthetic route of GLP-1R agonist Linker-payloads (LP34)

### Step 1: Preparation of [(2-chlorophenyl)-diphenyl-methyl]2-[[2-(9H-fluoren-9-ylmethoxycarbonylamino)acetyl]amino]acetate (LP34-2)

To a mixture LP34-1 (20 g, 21.01 mmol, 32.8% purity, 1 eq.) in DMF (200 mL) was shaked for 30 min and a solution of 2-[[2-(9H-fluoren-9-ylmethoxycarbonylamino)acetyl]amino]acetic acid (37.23 g, 105.06 mmol, 5 eq.) and DIPEA (27.16 g, 210.11 mmol, 36.60 mL, 10 eq.) in DMF (200 mL) was added. The resulting mixture was shaked for 12 h at 20° C. The resulting mixture was shaked for 12 h at 20° C. The mixture was added MeOH (100 mL) and shaked for another 2 h. The crude product WUXI-262-2 (26.68 g, crude) was used into the next step without further purification as a yellow solid.

### Step 2: Preparation of (4S)-4-[[(2S)-1-[(2S)-2-amino-4-methyl-pentanoyl]pyrrolidine-2-carbonyl]amino]-5-[[(1S,2R)-1-[[2-(carboxymethylamino)-2-oxo-ethyl]carbamoyl]-2-hydroxy-propyl]amino]-5-oxo-pentanoic acid (LP34-3)

The solid-phase peptide synthesis was carried on Liberty Lite—Automated Microwave Peptide Synthesizer. The LP34-2 Resin (0.43 mmol, 1 eq.) was swollen with DMF (10 mL) for 300S on standard. Following the standard operation on peptide synthesizer: a) De-protection: a solution of 20% piperidine/DMF (5 mL) was added to the resin vessel, agitated with N₂ for 2 min at 90° C. Then drained the vessel and washed with DMF (3 mL×3) at 20° C. b) Coupling (each amino acid reacted for triple with 5.0 eq.): a solution of amino acid (2.5 mmol, 5 eq.) in DMF (5 mL), DIC (2 mL) and oxyma (1 mL) were added to the vessel and agitated with N₂ for 10 min at 90° C. Repeat a) and b) for all amino acids. The resin was subjected to acidic cleavage by using TFA cocktail (TFA/TIPS/H₂O=95:2.5:2.5), then filtered and the filtrate was diluted with t-BuOMe to give a precipitate, which was centrifuged (5000 R) for 10 min to afford product P34-3 (0.6 g, crude, TFA) as a white solid. ¹H NMR (400 MHz, DMSO-d6) 13.13-11.71 (m, 2H), 8.52-7.97 (m, 7H), 7.85-7.59 (m, 1H), 5.06 (br s, 1H), 4.48-4.28 (m, 2H), 4.26-4.16 (m, 1H), 4.12 (br s, 1H), 4.04-3.94 (m, 1H), 3.90-3.68 (m, 5H), 2.35-1.72 (m, 9H), 1.64-1.44 (m, 2H), 1.03 (d, J=6.3 Hz, 3H), 0.96-0.87 (m, 6H).

### Step 3: Preparation of (4S)-5-[[(1S,2R)-1-[[2-(carboxymethylamino)-2-oxo-ethyl]carbamoyl]-2-hydroxy-propyl]amino]-4-[[(2S)-1-[(2S)-4-methyl-2-[3-[2-[2-[2-[2-[2-[2-(2-prop-2-ynoxyethoxy)ethoxy]ethoxy]ethoxy]ethoxy]ethoxy]ethoxy]propanoylamino]pentanoyl]pyrrolidine-2-carbonyl]amino]-5-oxo-pentanoic acid (LP34-5) (SEQ ID NOS 605-606, respectively, in order of appearance)

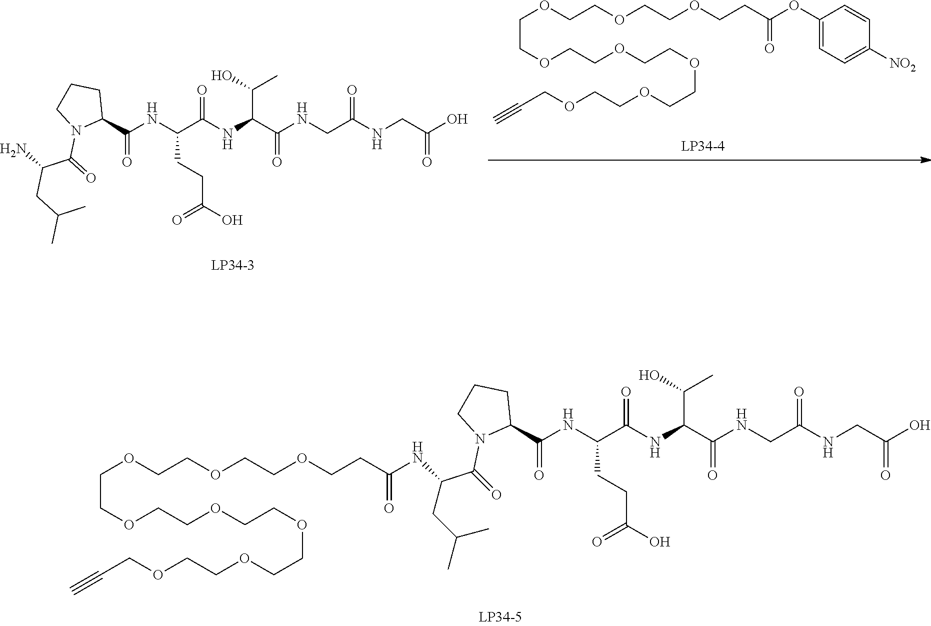 [SMILES: CC(C)C[C@H](N)C(=O)N1CCC[C@H]1C(=O)N[C@@H](CCC(=O)O)C(=O)N[C@H](C(=O)NCC(=O)NCC(=O)O)[C@@H](C)O ; C#CCOCCOCCOCCOCCOCCOCCOCCOCCC(=O)Oc1ccc([N+](=O)[O-])cc1 ; C#CCOCCOCCOCCOCCOCCOCCOCCOCCC(=O)N[C@@H](CC(C)C)C(=O)N1CCC[C@H]1C(=O)N[C@@H](CCC(=O)O)C(=O)N[C@H](C(=O)NCC(=O)NCC(=O)O)[C@@H](C)O]

To a solution of LP34-3 (45 mg, 65.54 μmol, 1 eq., TFA) and LP34-4 (36.54 mg, 65.54 μmol, 1 eq.) in DMF (2 mL) was added DIPEA (16.94 mg, 131.07 μmol, 22.83 μl, 2 eq.). The solution was stirred at 20° C. for 1 h. LCMS showed the desired product was observed. (ESI): RT=2.455 min, mass calcd. for C₄₇H₇₇N₁₉O₁₉N₆ 992.08 [M+H]⁺, found 991.6 [M+H]⁺. Reverse phase LCMS was carried out using a Chromolith Flash 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate3 μm, C18,2.1*30 mm; The mixture was diluted with CH3CN (2 mL). The residue was purified by prep-HPLC (TFA condition; column: Welch Xtimate C18 100*40 mm*3 μm; mobile phase: [water (0.075% TFA)-ACN]; B %: 15%-45%, 10 min). LP34-5 (50 mg, 49.54 μmol, 75.59% yield, 98.2% purity) was obtained as colorless oil confirmed by LCMS: ES17478-97-P1D2, HPLC: ES17478-97-p1C1 and HNMR: ES17478-97-P1B1.

(ESI): RT=3.158 min, mass calcd. for C₄₇H₇₇N₁₉O₁₉N₆ 992.08 [M+H]⁺, found 991.6 [M+H]+Reverse phase LCMS was carried out using a Chromolith Flash 1.5 ML/4 L TFA in water (solvent A) and 0.75 ML/4 L TFA in acetonitrile (solvent B), using the gradient 0%-60% (solvent B) over 6 minutes and holding at 60% for 0.5 minutes at a flow rate of 0.8 ml/min; Column: Xtimate3 μm, C18,2.1*30 mm;

HPLC: RT=3.69 min. HPLC conditions: Mobile Phase: 2.75 ML/4 L TFA in water (solvent A) and 2.5 ML/4 L TFA in acetonitrile (solvent B), using the elution gradient 1%-100% (solvent B) over 10 minutes and holding at 100% for 5 minutes at a flow rate of 1.5 ML/min; Column: Ultimate XB-C18.3 μm, 3.0*50 mm.

¹H NMR (400 MHz, DMSO-d6) 8.20-8.06 (m, 4H), 7.61 (d, J=8.0 Hz, 1H), 4.62-4.50 (m, 1H), 4.34 (br dd, J=4.4, 8.0 Hz, 1H), 4.31-4.24 (m, 1H), 4.19 (dd, J=4.0, 8.0 Hz, 1H), 4.14 (d, J=2.4 Hz, 2H), 4.04-3.96 (m, 1H), 3.82-3.73 (m, 4H), 3.60-3.47 (m, 34H), 2.41-2.25 (m, 4H), 1.99-1.82 (m, 4H), 1.80-1.69 (m, 1H), 1.67-1.56 (m, 1H), 1.50-1.35 (m, 2H), 1.32-1.22 (m, 1H), 1.03 (d, J=6.4 Hz, 3H), 0.88 (t, J=7.2 Hz, 6H).

### Step 4: Preparation of (4S)-4-[[(2R)-1-[(2S)-2-[3-[2-[2-[2-[2-[2-[2-[2-[[1-[5-[4-[4-[(2S)-3-[[(1S)-1-carbamoyl-4-(3,5-dimethylphenyl)butyl]amino]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]pentyl]triazol-4-yl]methoxy]ethoxy]ethoxy]ethoxy]ethoxy]ethoxy]ethoxy]ethoxy]propanoylamino]-4-methyl-pentanoyl]pyrrolidine-2-carbonyl]amino]-5-[[(1S,2R)-1-[[2-(carboxymethylamino)-2-oxo-ethyl]carbamoyl]-2-hydroxy-propyl]amino]-5-oxo-pentanoic acid (LP34)

To a solution of P8 (15 mg, 9.54 μmol, 1 eq.), LP34-5 (19.00 mg, 19.17 μmol, 2.01 eq.) in DMSO (0.8 mL) and H2O (0.8 mL) were added CuSO₄·5H₂O (2.38 mg, 9.54 μmol, 1 eq.), SODIUM ASCORBATE (1.89 mg, 9.54 μmol, 1 eq.) and 1-(1-benzyltriazol-4-yl)-N,N-bis[(1-benzyltriazol-4-yl)methyl]methanamine (2.5 mg, 4.77 μmol, 0.5 eq.). The resulting mixture was stirred at 25° C. for 3 h. LCMS showed the desired product was observed. (ESI): RT=3.677 min, mass calcd. for C₁₂₀H₁₇₅N₂₆O₃₆FH 2562.25[M+H]⁺, 1282.6 [M+2H]²⁺, found 1282.2 [M+2H]²⁺. Reverse phase LCMS was carried out using a Chromolith Flash 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate3 μm, C18,2.1*30 mm; The mixture was diluted with MeOH (2 mL), filtered and the filtrate was sent to Prep-HPLC. The residue was purified by prep-HPLC (TFA condition). Column: Welch Xtimate C18 100*40 mm*3 μm; mobile phase: [water (0.075% TFA)-ACN]; B %: 28%-58%, 8 min. LP34 (8.9 mg, 3.33 μmol, 34.85% yield, 96.3% purity) was obtained as a white solid.

LCMS RT=3.682 min, mass calcd. for C₁₂₀H₁₇₅N₂₆O₃₆FH 2562.25[M+H]⁺, 1282.6 [M+2H]²⁺, found 1282.2 [M+2H]²⁺. Reverse phase LCMS was carried out using a Chromolith Flash 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min; Column: Xtimate3 μm, C18,2.1*30 mm.

HPLC: RT=4.34 min. HPLC conditions: Mobile Phase: 2.75 ML/4 L TFA in water (solvent A) and 2.5 ML/4 L TFA in acetonitrile (solvent B), using the elution gradient 1%-100% (solvent B) over 10 minutes and holding at 100% for 5 minutes at a flow rate of 1.5 ML/min; Column: Ultimate XB-C18.3 μm, 3.0*50 mm.

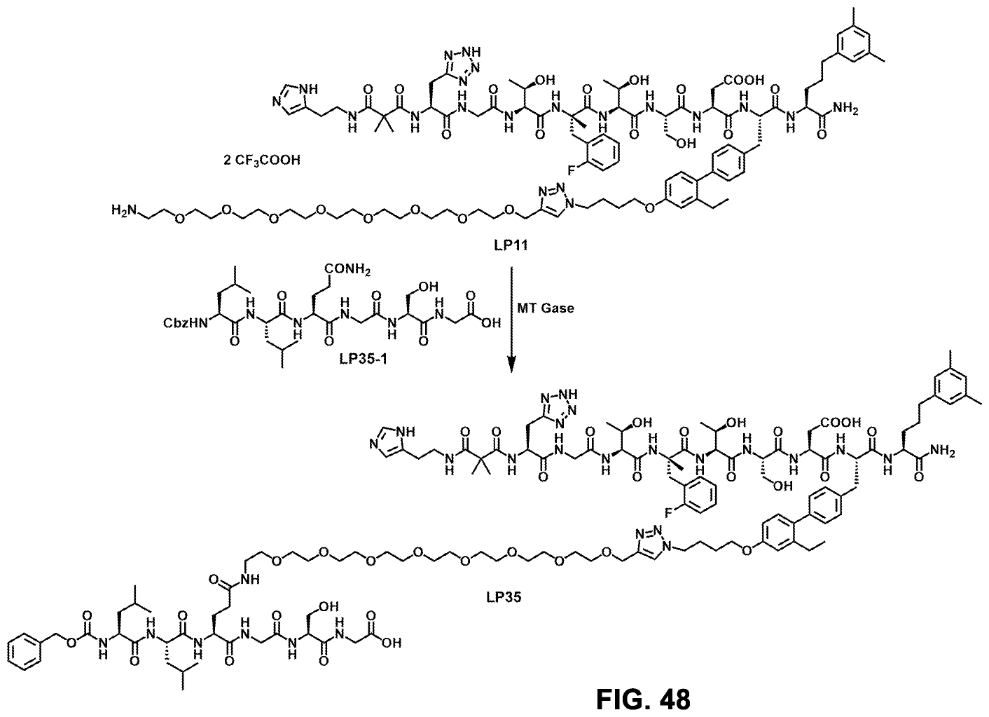

FIG. 48 depicts synthetic route of GLP-1R agonist Linker-payloads (LP35)

### Preparation of (3S)-4-[[(1S)-1-[[4-[4-[4-[4-[2-[2-[2-[2-[2-[2-[2-[2-[[(4S)-4-[[(2S)-2-[[(2S)-2-(benzyloxycarbonylamino)-4-methyl-pentanoyl]amino]-4-methyl-pentanoyl]amino]-5-[[2-[[(1S)-2-(carboxymethylamino)-1-(hydroxymethyl)-2-oxo-ethyl]amino]-2-oxo-ethyl]amino]-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[[(1S)-1-carbamoyl-4-(3,5-dimethylphenyl) butyl]amino]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP35)

A solution of LP11 (6.0 mg, 2.72 μmol, 1.0 eq., 2TFA) and PBS buffer (K-free, 100 mM, pH=7.20) (7.2 mL) was measured pH as 7.2. LP35-1 (9.62 mg, 13.59 μmol, 5.0 eq.) and MTGase (Ajinomoto-TI, 51.6 mg) were added. The resulting mixture was stirred at 37° C. for 16 h. The reaction progress was monitored by LCMS. Upon completion, the reaction mixture was quenched with aqueous AcOH solution (1% v/v, 7.2 mL). The obtained solid was rinsed with DMF/H₂O (3 mL, 1/1), and then the filtrate was purified by prep-HPLC (column: O-Xbridge C18 150*10 mm*5 μm; mobile phase: [water (0.075% TFA)-ACN]; B %: 10%-65%, 20 min) to afford LP35 (2.62 mg, 0.926 μmol, 34.1% yield, 98.3% purity, TFA salt) as a white solid.

LCMS: (ESI): RT=3.62 min, m/z calcd. for C₁₂₆H₁₈₄FN₂₇O₃₆ 1335.17 [M+2H]²⁺, found 1335.6; 100% purity at 220 nm. LCMS conditions: Xtimate C18 2.1*30 mm, 3 μm; 1.5 mL/4 L TFA in water (solvent A) and 0.75 mL/4 L TFA in acetonitrile (solvent B), using the elution gradient 10%-80% (solvent B) over 6 minutes and holding at 80% for 0.5 minutes at a flow rate of 0.8 mL/min.

UPLC: RT=7.15 min, 98.38% purity at 220 nm. UPLC method: Waters ACQUITY UPLC BEH C18 1.7 um, 2.1*100 mm; 0.05% TFA in 1 L water (solvent A) and 0.05% TFA in 1 L acetonitrile (solvent B), using the elution gradient 3%-100% (solvent B) over 11 minutes and holding at 100% for 4 minutes at a flow rate of 0.4 mL/minute.

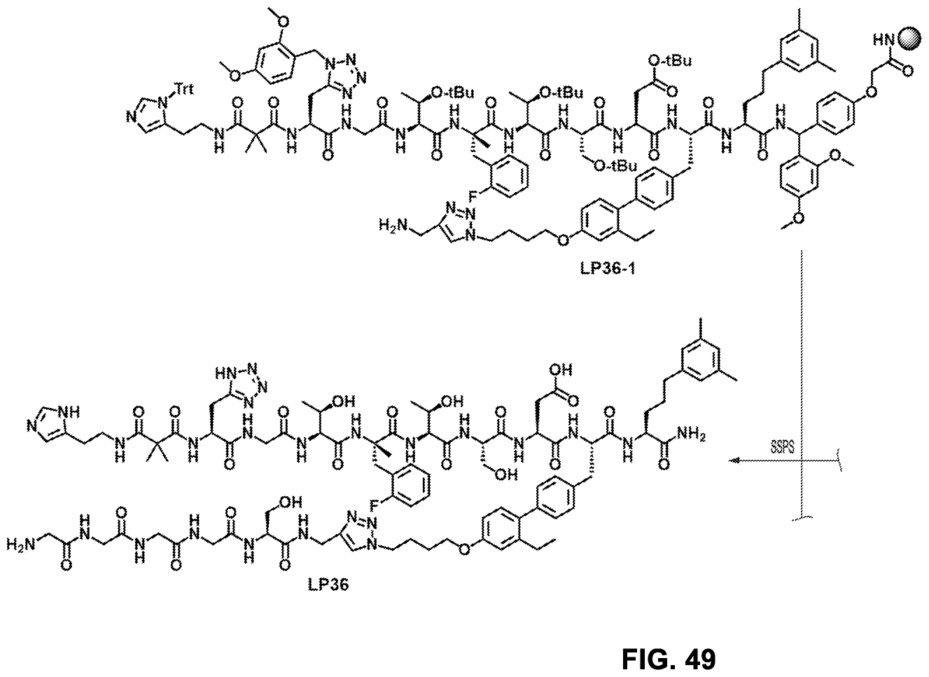

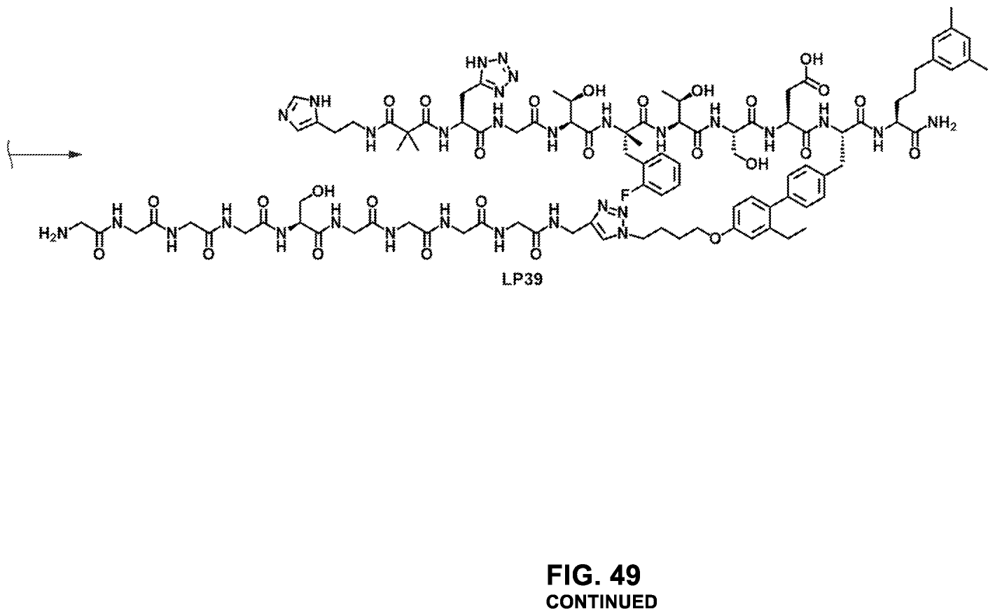

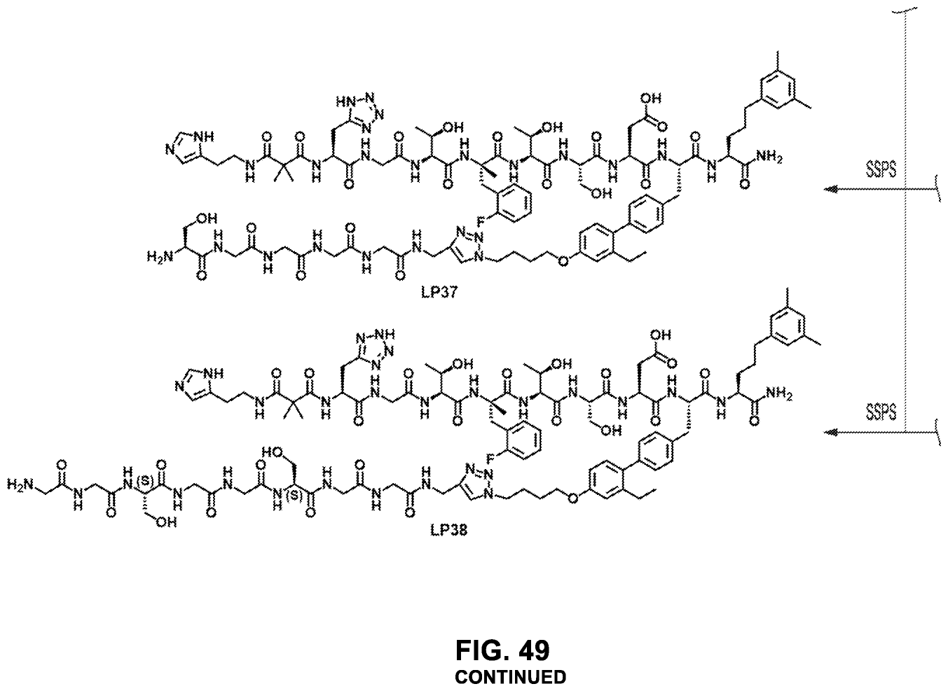

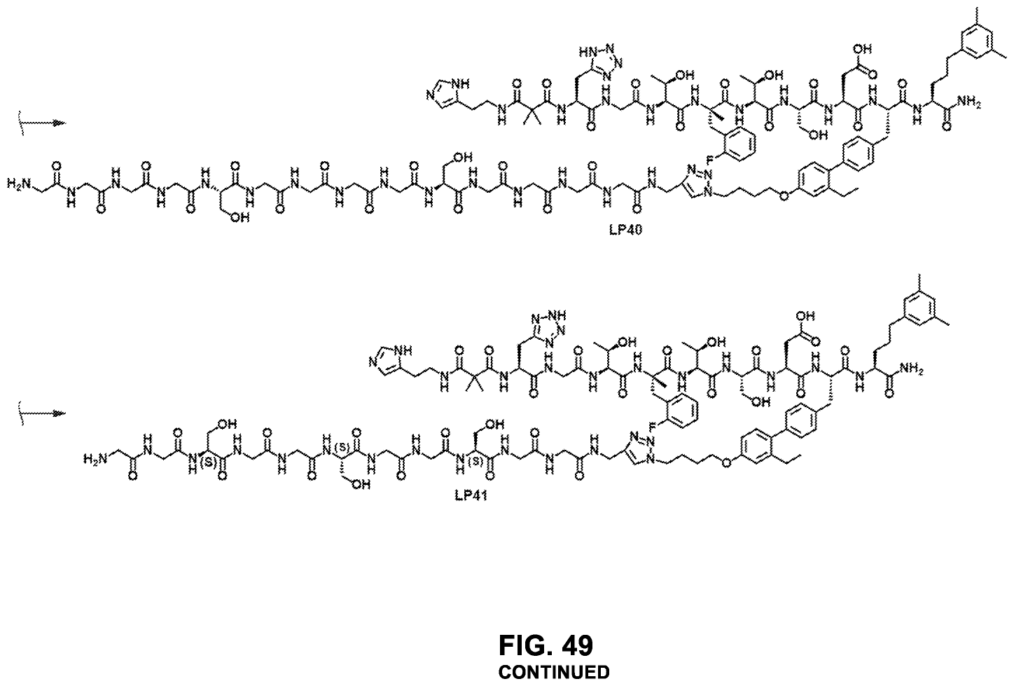

FIG. 49 depicts synthetic route of GLP-1R agonist Linker-payloads (LP36, LP37, LP38, LP39, LP40, LP41)

### 5.36 Preparation of (3S)-4-[[(1S)-1-[[4-[4-[4-[4-[[[(2S)-2-[[2-[[2-[[2-[(2-aminoacetyl)aminol acetyl]amino]acetyl]amino]acetyl]amino]-3-hydroxy-propanoyl]amino]methyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(1H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP36)

The solid-phase peptide synthesis was carried out on the Liberty Lite—Automated Microwave Peptide Synthesizer. The LP36-1 (0.43 mmol, 1 eq.) was swollen with DMF (10 mL) for 300S on standard. Following the standard operation on peptide synthesizer: a) De-protection: a solution of 20% piperidine/DMF (5 mL) was added to the resin vessel, agitated with N₂ for 2 min at 90° C. Then drained the vessel and washed with DMF (3 mL×3) at 20° C. b) Coupling (each amino acid reacted for triple with 5.0 eq.): a solution of amino acid (2.5 mmol, 5 eq.) in DMF (5 mL), DIC (2 mL) and oxyma (1 mL) were added to the vessel and agitated with N₂ for 10 min at 90° C. Repeat a) and b) for all amino acids. The resin was subjected to acidic cleavage by using TFA cocktail (TFA/TIPS/H₂O=95:2.5:2.5), then filtered and the filtrate was diluted with t-BuOMe to give a precipitate, which was centrifuged (5000 R) for 10 min to give the crude product.

LP36 was prepared as described in the general procedure of SPPS. The crude product was purified by prep-HPLC (column: Welch Xtimate C18 100*40 mm*3 μm; mobile phase: [water (0.075% TFA)-ACN]; B %: 0%-40%,15 min) to afford pure product LP36 (13.9 mg, 6.25 μmol, 1.44% yield, 97.58% purity, 2TFA) as a white solid.

LCMS: (ESI): Rt=2.715 min, m/z calcd. For C₈₉H₁₂₃FN₂₆O₂₃, 971.46, [M+2H]²⁺; found 971.9 [M+2H]²⁺; Reverse phase LCMS was carried out using Chromolith Flash RPC1825-3 mm, with a flow rate of 0.8 ml/min, eluting with a gradient of 10% to 80% acetonitrile containing 0.02% TFA (solventB) and water containing 0.04% TFA (solvent A).

HPLC: RT=6.62 min. HPLC Conditions: Mobile phase:1.0% ACN in water (0.1% TFA) to 5% ACN in water (0.1% TFA) in 1 minutes; then from 5% ACN in water(0.1% TFA) to 100% ACN (0.1% TFA) in 5 minutes; hold at 100% ACN (0.1% TFA) for 2 minutes; back to 1.0% ACN in water (0.1% TFA) at 8.01 minutes, and hold two minutes. Flow rate:1.2 ml/min Column: Ultimate XB-C18.3 μm, 3.0*50 mm

HRMS (ESI): m/z calcd for C₈₉H₁₂₁FN₂₆O₂₃ 1941.91 [M+H]⁺, 971.46 [M+2H]²⁺, found 1942.9299 [M+H]⁺, 971.9736 [M+2H]²⁺.

UPLC: RT=5.824 min. conditions: Mobile Phase: 0.05% TFA in 1 L water (solvent A) and 0.05% TFA in 1 L acetonitrile (solvent B), using the elution gradient 30%-80% (solvent B) over 10 minutes and holding at 80% for 5 minutes at a flow rate of 0.4 mL/minute; Column: Waters ACQUITY UPLC HSS T3 1.8 um, 2.1*100 mm;

5.37 LP37 was obtained as the same with LP36. The crude product was purified by prep-HPLC (column: Welch Xtimate C18 100*40 mm*3 μm; mobile phase: [water (0.075% TFA)-ACN]; B %: 0%-40%,15 min) to afford pure product LP37 (5 mg, 2.50 μmol, 1.25% yield, 97% purity) as a white solid.

LCMS: (ESI): Rt=2.800 min, mass calcd. for C₈₉H₁₂₁FN₂₆O₃; found 971.45 [M/2+H]⁺ found 971.0; Reverse phase LCMS was carried out using Chromolith Flash RPC1825-3 mm, with a flow rate of 0.8 ml/min, eluting with a gradient of 10% to 80% acetonitrile containing 0.02% TFA (solvent B) and water containing 0.04% TFA (solvent A).

HPLC: RT=6.61 min. Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min; Column: WELCH Ultimate LP-C18 150*4.6 mm 5 μm; Wavelength: UV 220 nm&215 nm&254 nm; Column temperature: 40° C.

HRMS-TOF: C₈₉H₁₂₂N₂₆O₂₃F [M+H]=1942.9573. The mobile phase: 0.1% FA in water (solvent A) and 0.05% FA in ACN (solvent B); Elution Gradient: 5%-95% (solvent B) over 1.3 minutes and holding at 95% for 0.7 minutes at a flow rate of 1.2 ml/minute; Column: Agilent Poroshell HPH-C182.7 um, 2.1*50 mm; Ion Source: AJS ESI source; Ion Mode: Positive; Nebulization Gαs: Nitrogen; Drying Gαs (N2) Flow: 8 L/min; Nebulizer Pressure: 35 psi; Gas Temperature: 325oC; Sheath gas Temperature: 350oC; Sheath gas flow: 11 L/min; Capillary Voltage: 3.5 KV; Fragmentor Voltage: 300 V.

UPLC: RT=4.595 min, mass calcd. Mobile Phase: 0.05% TFA in 1 L water (solvent A) and 0.05% TFA in 1 L acetonitrile (solvent B), using the elution gradient 0%-95% (solvent B) over 6 minutes and holding at 95% for 4 minutes at a flow rate of 0.6 mL/minute; Column: Waters ACQUITY UPLC HSS T3 2.1*50 mm,1.8 μm; Column temp:40° C.

5.38 LP38 was obtained as the same with LP36. The crude product was purified by prep-HPLC (column: Welch X timate C18 100*40 mm*3 μm; mobile phase: [water (0.075% TFA)-ACN]; B %: 15%-45%, 15 min) to afford pure product LP38 (52.17 mg, 8.87 μmol, 2.53% yield, 95% purity) as a white solid.

LCMS: (ESI): Rt=2.723 min, mass calcd. for C₉₆H₁₃₄FN₂₉O₂₇ [M+2H]²⁺1071.99, C₉₆H₁₃₅FN₂₉O₂₇ [M+3H]³⁺714.99; found 1072.40 [M+2H]²⁺ found 715.30 [M+3H]³⁺; Reverse phase LCMS was carried out using Chromolith Flash RP-C18 25-3 mm, with a flow rate of 0.8 ml/min, eluting with a gradient of 10% to 80% acetonitrile containing 0.02% TFA (solvent B) and water containing 0.04% TFA (solvent A).

HPLC RT=9.00 min, 95% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min;

5.39 LP39 was obtained as the same with LP36. The crude product was purified by prep-HPLC (column: Welch Xtimate C18 100*40 mm*3 μm; mobile phase: [column: Waters X bridge BEH C18 100*25 mm*5 μm; mobile phase: [water (0.05% NH3H2O)-ACN]; B %: 5%-32%, 11 min) o afford pure product LP39 (20 mg, 8.75 μmol, 2.50% yield, 95% purity) as a white solid.

LCMS: (ESI): Rt=2.711 min, mass calcd. for C₉₇H₁₃₅FN₃₀O₂₇ [M+2H]²⁺1085.49, C₉₇H₁₃₆FN₃₀O₂₇ [M+3H]³⁺724.30; found 1085.90 [M+2H]²⁺ found 725.30[M+3H]3+; Reverse phase LCMS was carried out using Chromolith Flash RP-C1825-3 mm, with a flow rate of 0.8 ml/min, eluting with a gradient of 10% to 80% acetonitrile containing 0.02% TFA (solvent B) and water containing 0.04% TFA (solvent A).

HPLC RT=8.99 min, 95% purity. HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min;

5.40 LP40 was obtained as the same with LP36. The crude product was purified by prep-HPLC (column: Welch X timate C18 100*40 mm*3 μm; mobile phase: [water (0.075% TFA)-ACN]; B %: 6%-46%, 20 min) to afford pure product LP40 (3.5 mg, 1.38 μmol, 3.94e-1% yield, 97.99% purity) as a white solid.

LCMS: (ESI): Rt=2.740 min, mass calcd. for C₁₀₈H₁₅₂FN₃₅O₃₃ [M+2H]²⁺1243.05, C₁₀₈H₁₅₃FN₃₅O₃₃ [M+3H]³⁺829.03; found 829.4[M+2H]²⁺ found 1243.30[M+3H]³⁺; Reverse phase LCMS was carried out using X Bridge C18 3.5 μm 2.1*30 mm, with a flow rate of 0.8 ml/min, eluting with a gradient of 10% to 80% acetonitrile (solvent B) and NH3·H2O in water (solvent A).

HPLC RT=6.50 min, 97.99% purity; HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min;

5.41 LP41 was obtained as the same with LP36. The crude product was purified by prep-HPLC (column: Welch Xtimate C18 100*40 mm*3 μm; mobile phase: [water (0.075% TFA)-ACN]; B %: 6%-46%, 20 min) to afford pure product LP41 (4.5 mg, 1.89 μmol, 0.54% yield, 98.52% purity) as a white solid.

LCMS: (ESI): Rt=2.590 min, mass calcd. for C₁₀₃H₁₄₅FN₃₂O₃₁ [M+2H]²⁺1173.21, C₁₀₃H₁₄₆FN₃₂O₃₁ [M+3H]³⁺782.47; found 1172.90 [M+2H]²⁺ found 782.30 [M+3H]³⁺; Reverse phase LCMS was carried out using X Bridge C18 3.5 μm 2.1*30 mm, with a flow rate of 0.8 ml/min, eluting with a gradient of 10% to 80% acetonitrile (solvent B) and NH3·H2O in water (solvent A).

HPLC RT=6.54 min, 98.52% purity; HPLC method A: Column: YMC-Pack ODS-A 150*4.6 mm, 5 μm; 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

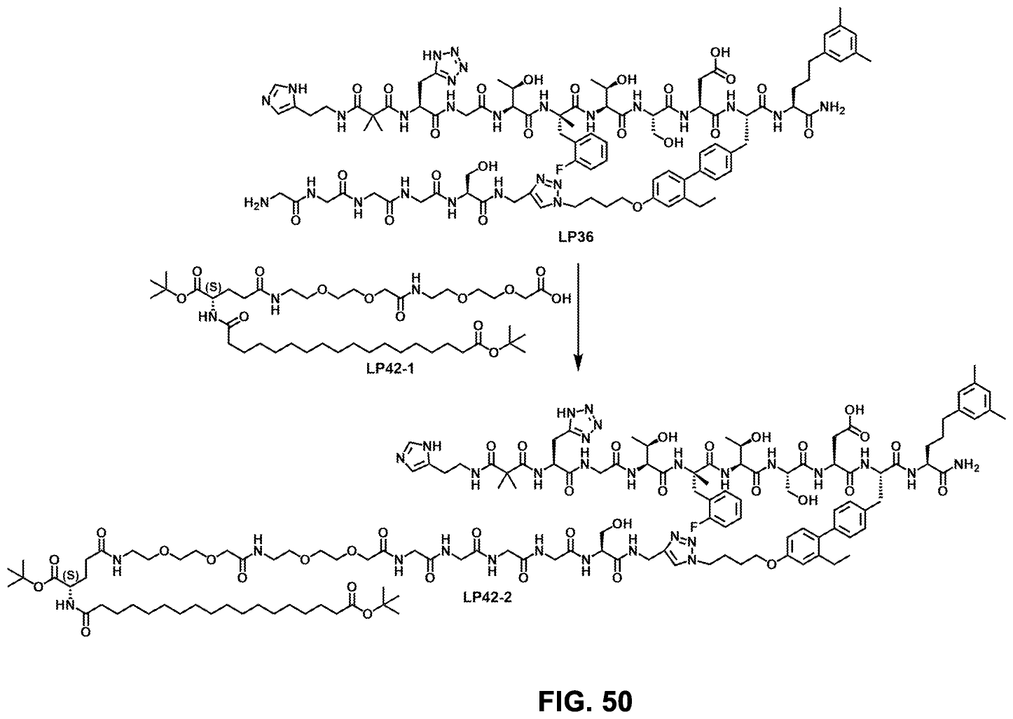

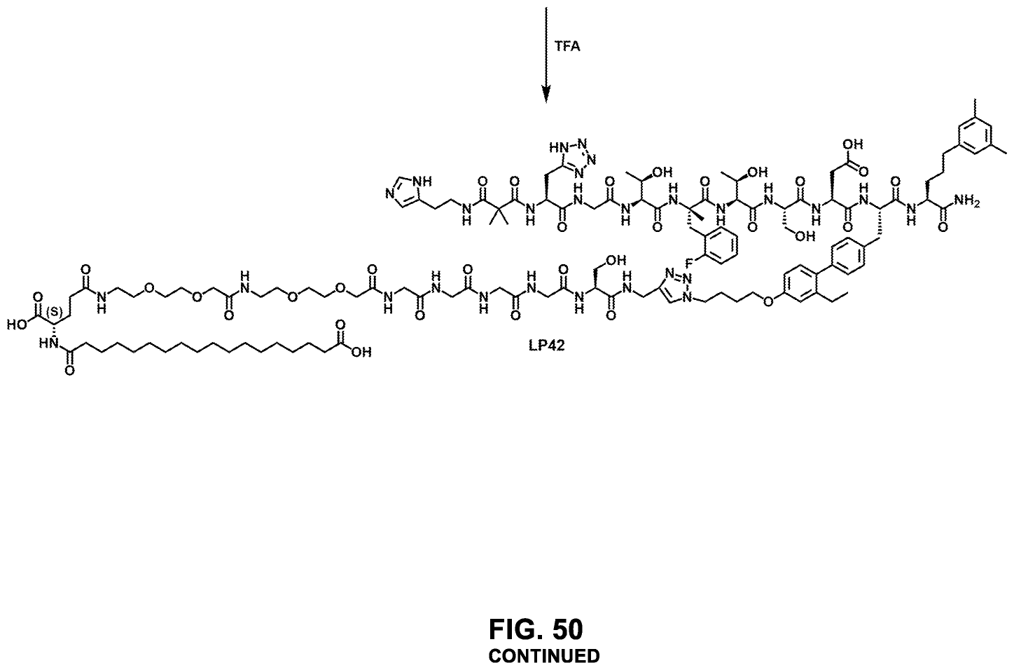

FIG. 50 depicts synthetic route of GLP-1R agonist Linker-payloads (LP42)

### Step 1: Preparation of (3S)-4-[[(1S)-1-[[4-[4-[4-[4-[[[(2S)-2-[[2-[[2-[[2-[[2-[[2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetyl]amino]acetyl]amino]acetyl]amino]acetyl]amino]acetyl]amino]-3-hydroxy-propanoyl]amino]methyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(1H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP42-2)

To a solution of LP42-1 (45.75 mg, 54.07 μmol, 1.5 eq.) and HATU (16.45 mg, 43.25 μmol, 1.2 eq.) in DMF (2 mL) was added DIPEA (13.98 mg, 108.13 μmol, 18.83 μL, 3 eq). The mixture was stirred at 20° C. for 0.1 hr. LP36 (70 mg, 36.04 μmol, 1 eq.) was added and the mixture was stirred at 20° C. for 1 hr. LCMS showed the reaction converted completely. The reaction mixture was added into MTBE (40 mL) dropwise. The precipitated yellow solid was collected by centrifuged. LP42-2 (110 mg, 25.49 μmol, 70.71% yield, 64.18% purity) was obtained as a light-yellow solid.

LCMS (ESI): RT=4.670 min, m/z calcd. For C132H199FN29035 1385.23 [M+2H]2+; m/z found 1385.8; LCMS Conditions: Mobile Phase:1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min. Column: Xtimate C18 2.1*30 mm, 3 μm.

### Step 2: Preparation of 18-[[(1S)-4-[2-[2-[2-[2-[2-[2-[[2-[[2-[[2-[[2-[[(1S)-2-[[1- [4-[4-[4-[(2S)-3-[[(1S)-1-carbamoyl-4-(3,5-dimethylphenyl) butyl]amino]-2-[[f(2S)-3-carboxy-2-[[f(2S)-2-[[f(2S, 3R)-2-[[f(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(1H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]butyl]triazol-4-yl]methylamino]-1-(hydroxymethyl)-2-oxo-ethyl]amino]-2-oxo-ethyl]amino]-2-oxo-ethyl]amino]-2-oxo-ethyl]amino]-2-oxo-ethyl]amino]-2-oxo-ethoxy]ethoxy]ethylamino]-2-oxo-ethoxy]ethoxy]ethylamino]-1-carboxy-4-oxo-butyl]amino]-18-oxo-octadecanoic acid (LP42)

To a solution of LP42-2 (110 mg, 25.49 μmol, 64.18% purity, 1 eq.) in DCM (1 mL) was added TFA (1.54 g, 13.51 mmol, 1 mL, 529.95 eq.). The mixture was stirred at 20° C. for 1 hr. LCMS showed the reaction converted completely. The reaction mixture was purified by prep-HPLC (column: Welch Xtimate C18 100*40 mm*3 μm; mobile phase: [water (0.075% TFA)-ACN]; B %: 10%-50%, 40 min) to give the dedired compound LP42 (24 mg, 8.68 μmol, 17.03% yield, 96.11% purity) as a white solid.

LCMS: (ESI): Rt=3.943 min, m/z calcd. For C₁₂₄H₁₈₄FN₂₉O₃₅ 1329.17 [M+2H]²⁺; m/z found 1329.6; Reverse phase LCMS was carried out using Chromolith Flash RPC1825-3 mm, with a flow rate of 0.8 ml/min, eluting with a gradient of 10% to 80% acetonitrile containing 0.02% TFA (solvent B) and water containing 0.04% TFA (solvent A).

HPLC: RT=8.52 min. HPLC conditions: Mobile phase: 1.0% ACN in water (0.1% TFA) to 5% ACN in water (0.1% TFA) in 1 minutes; then from 5% ACN in water (0.1% TFA) to 100% ACN (0.1% TFA) in 5 minutes; hold at 100% ACN (0.1% TFA) for 2 minutes; back to 1.0% ACN in water (0.1% TFA) at 8.01 minutes, and hold two minutes. Flow rate: 1.2 ml/min. Column: Ultimate XB-C18, 3 μm, 3.0*50 mm

HRMS (ESI): m/z calcd for C₁₂₄H₁₈₃FN₂₉O₃₅ 2657.33 [M+H]⁺, 1329.665 [M+2H]²⁺, found 2657.33 [M+H]⁺, 1329.6703 [M+2H]²⁺.

UPLC: RT=5.411 min. conditions: Mobile Phase: 0.05% TFA in 1 L water (solvent A) and 0.05% TFA in 1 L acetonitrile (solvent B), using the elution gradient 0%-95% (solvent B) over 6 minutes and holding at 95% for 4 minutes at a flow rate of 0.4 mL/minute; Column: Waters ACQUITY UPLC HSS T3 1.8 um, 2.1*100 mm.

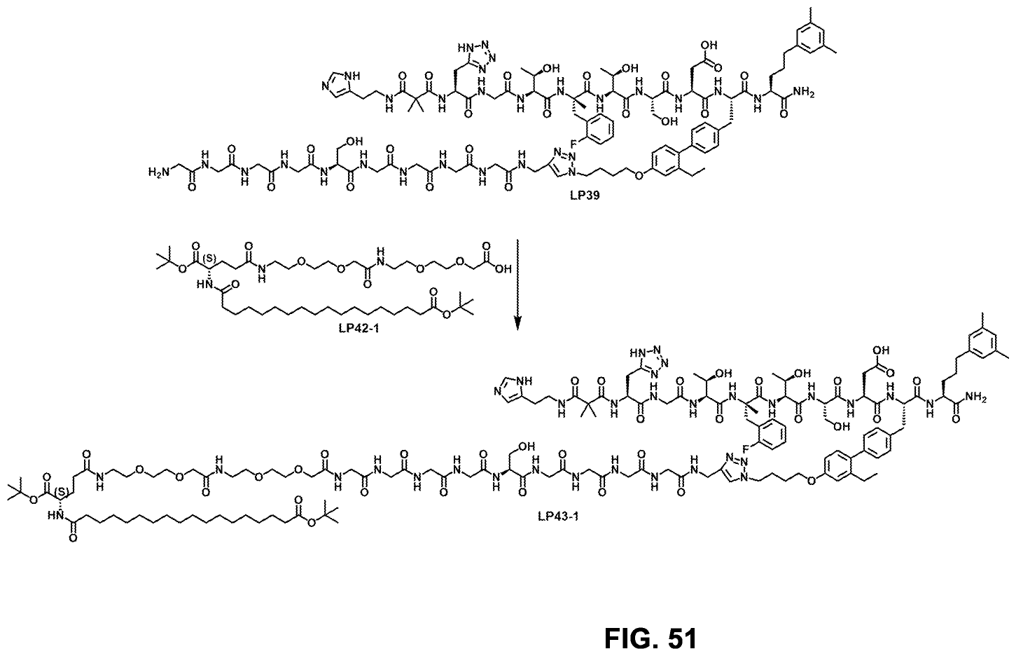

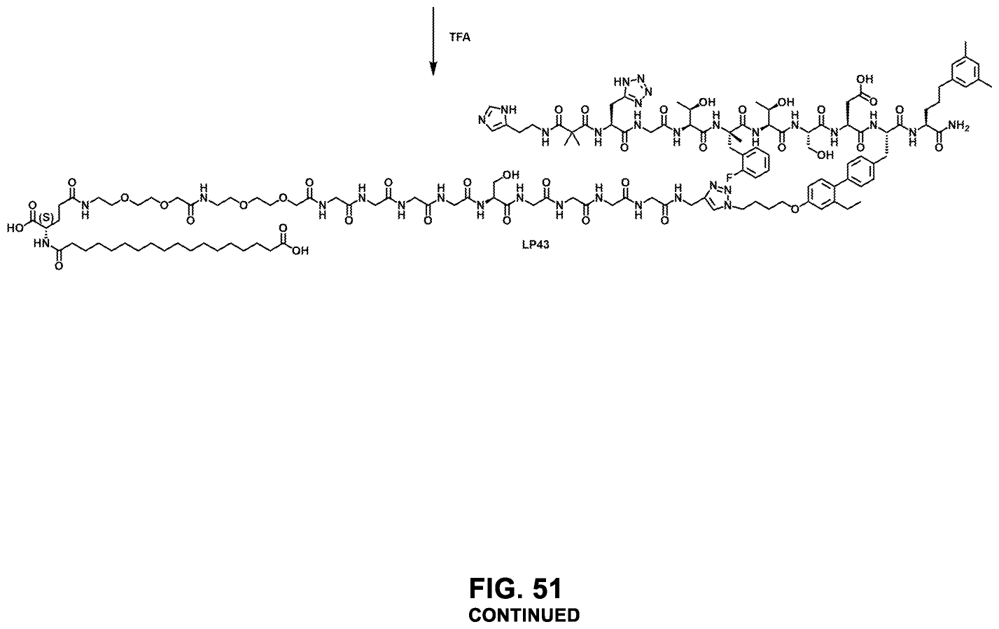

FIG. 51 depicts synthetic route of GLP-1R agonist Linker-payloads (LP43)

### Step 1: Preparation of (3S)-4-[[(1S)-1-[[4-[4-[4-[4-[[[2-[[2-[[2-[[2-[[(2S)-2-[[2-[[2-[[2-[[2-[[2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetyl]amino]acetyl]amino]acetyl]amino]acetyl]amino]acetyl]amino]-3-hydroxy-propanoyl]amino]acetyl]amino]acetyl]amino]acetyl]amino]acetyl]amino]methyl]triazol-1-yl]butoxy]-2-ethyl-phenyl]phenyl]methyl]-2-[[(1S)-1-carbamoyl-4-(3,5-dimethylphenyl)butyl]amino]-2-oxo-ethyl]amino]-3-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(1H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP43-1)

To a solution of LP42-1 (41 mg, 18.89 μmol, 1 eq.) and HATU (8.62 mg, 22.67 μmol, 1.2 eq.) in DMF (0.3 mL) was added DIPEA (7.32 mg, 56.67 μmol, 9.87 μL, 3 eq). The mixture was stirred at 20° C. for 0.1 hr. LP39 (23.98 mg, 28.34 μmol, 1.5 eq.) was added and the mixture was stirred at 20° C. for 1 hr. LCMS showed the reaction converted completely. The reaction mixture was added into MTBE (40 mL) dropwise. The precipitated yellow solid was collected by centrifuged. LP43-1 (80 mg, 12.56 μmol, 66.49% yield, 47.08% purity) as a light yellow solid.

LCMS (ESI): RT=4.603 min, m/z calcd. For C₁₄₀H₂₁₃FN₃₃O₃₉ 999.85 [M+3H]³⁺; m/z found 1000.3; LCMS Conditions: Mobile Phase: 1.5 ML/4 L TFA in water (solvent A) and 0.75 ML/4 L TFA in acetonitrile (solvent B), using the gradient 10%-80% (solvent B) over 6 minutes and holding at 80% for 0.5 minutes at a flow rate of 0.8 ml/min. Column: Xtimate C18 2.1*30 mm, 3 μm.

### Step 3: Preparation of 18-[[(1S)-4-[2-[2-[2-[2-[2-[2-[[2-[[2-[[2-[[2-[[(1S)-2-[[2- [[2-[[2-[[2-[[1-[4-[4-[4-[(2S)-3-[[(1S)-1-carbamoyl-4-(3,5-dimethylphenyl)butyl]amino]-2-[[(2S)-3-carboxy-2-[[(2S)-2-[[(2S,3R)-2-[[(2S)-3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(1H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]butyl]triazol-4-yl]methylamino]-2-oxo-ethyl]amino]-2-oxo-ethyl]amino]-2-oxo-ethyl]amino]-2-oxo-ethyl]amino]-1-(hydroxymethyl)-2-oxo-ethyl]amino]-2-oxo-ethyl]amino]-2-oxo-ethyl]amino]-2-oxo-ethyl]amino]-2-oxo-ethyl]amino]-2-oxo-ethoxy]ethoxy]ethylamino]-2-oxo-ethoxy]ethoxy]ethylamino]-1-carboxy-4-oxo-butyl]amino]-18-oxo-octadecanoic acid (LP43)

To a solution of LP43-1 (80 mg, 12.56 μmol, 47.08% purity, 1 eq.) in DCM (0.7 mL) was added TFA (2.31 g, 20.26 mmol, 1.5 mL, 1612.83 eq). The mixture was stirred at 20° C. for 3 hr. LCMS showed the reaction converted completely. The residue was purified by prep-HPLC (column: 0-phenomenex clarity RP 150*10 mm*5 μm; mobile phase: [water (0.075% TFA)-ACN]; B %: 30%-52%, 20 min) to provide the desired compound LP43 (1.8 mg, 0.571 μmol, 4.55% yield, 91.60% purity) as a white solid.

LCMS: (ESI): RT=3.883 min, m/z calcd. For C₁₃₂H₁₉₆FN₃₃O₃₉ 1443.21 [M+2H]²⁺; m/z found 1443.8; Reverse phase LCMS was carried out using Chromolith Flash RPC1825-3 mm, with a flow rate of 0.8 ml/min, eluting with a gradient of 10% to 80% acetonitrile containing 0.02% TFA (solventB) and water containing 0.04% TFA (solvent A).

HPLC: RT=8.30 min. HPLC conditions: Mobile phase: 1.0% ACN in water (0.1% TFA) to 5% ACN in water (0.1% TFA) in 1 minutes; then from 5% ACN in water (0.1% TFA) to 100% ACN (0.1% TFA) in 5 minutes; hold at 100% ACN (0.1% TFA) for 2 minutes; back to 1.0% ACN in water (0.1% TFA) at 8.01 minutes, and hold two minutes. Flow rate: 1.2 ml/min Column: Ultimate XB-C18, 3 μm, 3.0*50 mm.

HRMS (ESI): m/z calcd for C₁₃₂H₁₉₅FN₃₃O₃₉ 2885.42 [M+H]⁺, 1442.21 [M+2H]²⁺, found 1442.21 [M+H]⁺, 1443.7161 [M+2H]²⁺.

UPLC: RT=5.316 min. conditions: Mobile Phase: 0.05% TFA in 1 L water (solvent A) and 0.05% TFA in 1 L acetonitrile (solvent B), using the elution gradient 0%-95% (solvent B) over 10 minutes and holding at 80% for 5 minutes at a flow rate of 0.4 mL/minute; Column: Waters ACQUITY UPLC HSS T3 1.8 um, 2.1*100 mm.

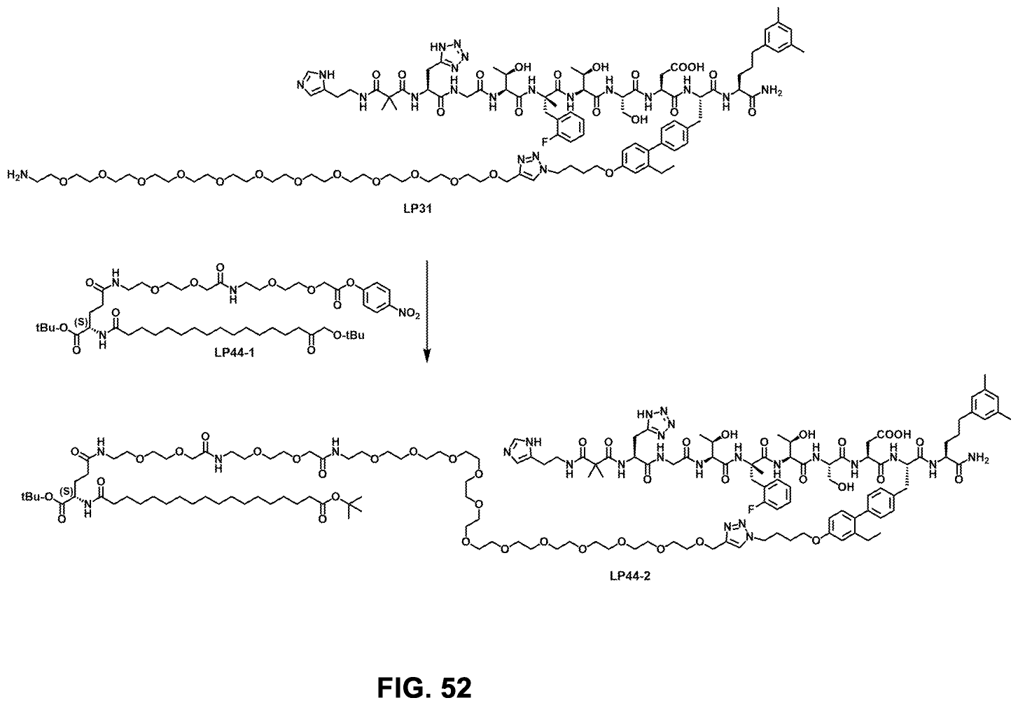

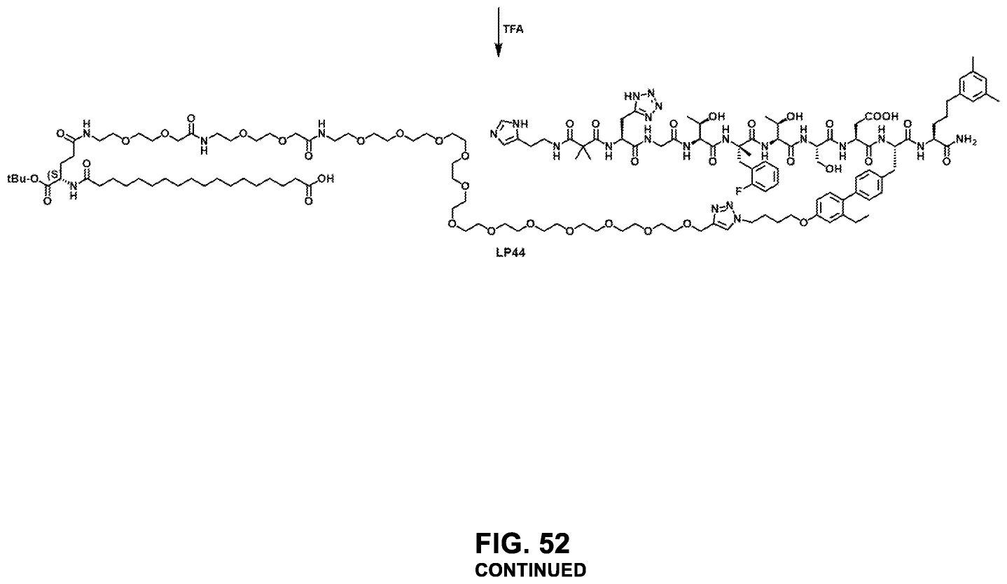

FIG. 52 depicts synthetic route of GLP-1R agonist Linker-payloads (LP44)

### Step 1: Preparation of (8S,14S,17S,20S,23S,26S)-8-((1H-tetrazol-5-yl)methyl)-26-(((S)-1-(((S)-1-amino-5-(3,5-dimethylphenyl)-1-oxopentan-2-yl)amino)-3-(4′-(4-(4-((S)-60-(tert-butoxycarbonyl)-81,81-dimethyl-39,48,57,62,79-pentaoxo-2,5,8,11,14,17,20,23,26,29,32,35,41,44,50,53,80-heptadecaoxa-38,47,56,61-tetraazadooctacontyl)-1H-1,2,3-triazol-1-yl)butoxy)-2′-ethyl-[1,1′-biphenyl]-4-yl)-1-oxopropan-2-yl)carbamoyl)-17-(2-fluorobenzyl)-14,20-bis((R)-1-hydroxyethyl)-23-(hydroxymethyl)-1-(1H-imidazol-5-yl)-5,5,17-trimethyl-4,6,9,12,15,18,21,24-octaoxo-3,7,10,13,16,19,22,25-octaazaoctacosan-28-oic acid (LP44-2)

To a solution of LP30 (40 mg, 18.56 μmol, 1 eq.) in DMF (1 mL) were added DIPEA (7.2 mg, 55.68 μmol, 10 μL, 3 eq.) and LP44-1 (22 mg, 22.27 μmol, 1.2 eq.). The resulting mixture was stirred at 20° C. for 0.5 h. LCMS showed the reaction was complete and the desired product was observed. The mixture was poured into ice-cooled MTBE, then filtered and the filter-cake was dried in vacuum. LP44-2 (55 mg, crude) was obtained as a colorless oil.

LCMS (ESI): RT=6.396 min, mass calcd. for C₁₄₅H₂₃₀FN₂₄O₄₁ 2982.66 [M+H]⁺, 746.44 [M+4H]⁴⁺, found 747.2 [M+4H]⁴⁺. Reverse phase LCMS was carried out using a Chromolith Flash 1.5 ML/4 L TFA in water (solvent A) and 0.75 ML/4 L TFA in acetonitrile (solvent B), using the gradient 10-80% (solvent B) over 6 minutes and holding at 80% for 0.5 minutes at a flow rate of 0.8 ml/min; Column: Xtimate3 μm, C18,2.1*30 mm.

### Step 2: Preparation of 18-[[(1S)-4-[2-[2-[2-[2-[2-[2-[2-[2-[2-[2-[2-[2-[2-[2-[2-[2-[2-[2-[[1-[4-[4-[4-[(2S)-3-[[(1S)-1-carbamoyl-4-(3,5-dimethylphenyl)butyl]amino]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl) ethylamino]-2,2-dimethyl-3-oxo-propanoyl]amino]-3-(1H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]butyl]triazol-4-yl]methoxy]ethoxy]ethoxy]ethoxy]ethoxy]ethoxy]ethoxy]ethoxy]ethoxy]ethoxyl ethoxy]ethoxy]ethylamino]-2-oxo-ethoxy]ethoxy]ethylamino]-2-oxo-ethoxy]ethoxy]ethylamino]-1-carboxy-4-oxo-butyl]amino]-18-oxo-octadecanoic acid (LP44-2)

A solution of LP44-2 (55 mg, 18.26 μmol, 1 eq.) in TFA (0.5 mL) and DCM (0.5 mL) was stirred at 20° C. for 1 h. LCMS showed the reaction completed and the desired product was observed. (ESI): RT=4.219 min, mass calcd. for C₁₃₇H₂₁₄FN₂₄O₄₁ 2870.53[M+H]⁺, 1436.27[M+2H]²⁺, found 1436.2 [M+2H]²⁺. Reverse phase LCMS was carried out using a Chromolith Flash 1.5 ML/4 L TFA in water (solvent A) and 0.75 ML/4 L TFA in acetonitrile (solvent B), using the gradient 10-80% (solvent B) over 6 minutes and holding at 80% for 0.5 minutes at a flow rate of 0.8 ml/min; Column: Xtimate 3 μm, C18, 2.1*30 mm. The reaction solution was concentrated in vacuum. The residue was purified by prep-HPLC (TFA condition; column: O-phenomenex clarity RP 150*10 mm*5 μm; mobile phase: [water (0.075% TFA)-ACN]; B %: 40%). The desired compound LP44 (12 mg, 4.05 μmol, 22.19% yield, 96.97% purity) was obtained as a white solid.

LCMS (ESI): RT=4.243 min, mass calcd. for C₁₃₇H₂₁₄FN₂₄O₄₁ 2870.53 [M+H]⁺, 1436.27 [M+2H]²⁺, found 1436.4 [M+2H]²⁺. Reverse phase LCMS was carried out using a Chromolith Flash 1.5 ML/4 L TFA in water (solvent A) and 0.75 ML/4 L TFA in acetonitrile (solvent B), using the gradient 10-80% (solvent B) over 6 minutes and holding at 80% for 0.5 minutes at a flow rate of 0.8 ml/min; Column: Xtimate3 μm, C18, 2.1*30 mm.

HPLC RT=9.13 min. HPLC conditions: Mobile Phase: 2.75 ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ML/min; Column: Ultimate XB-C18.3 μm, 3.0*50 mm.

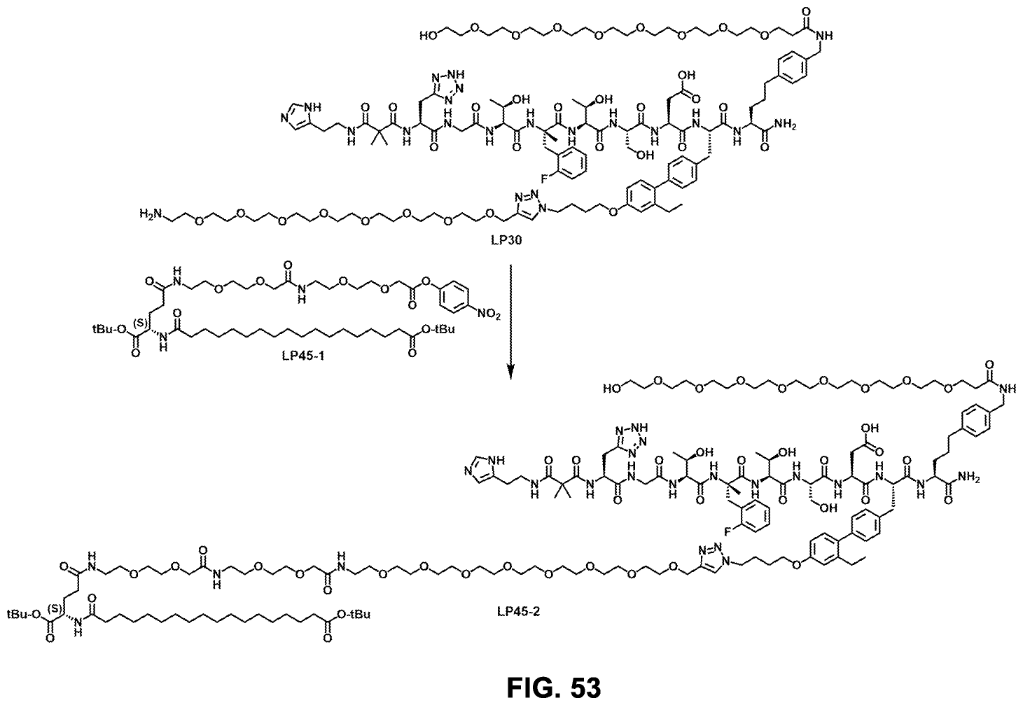

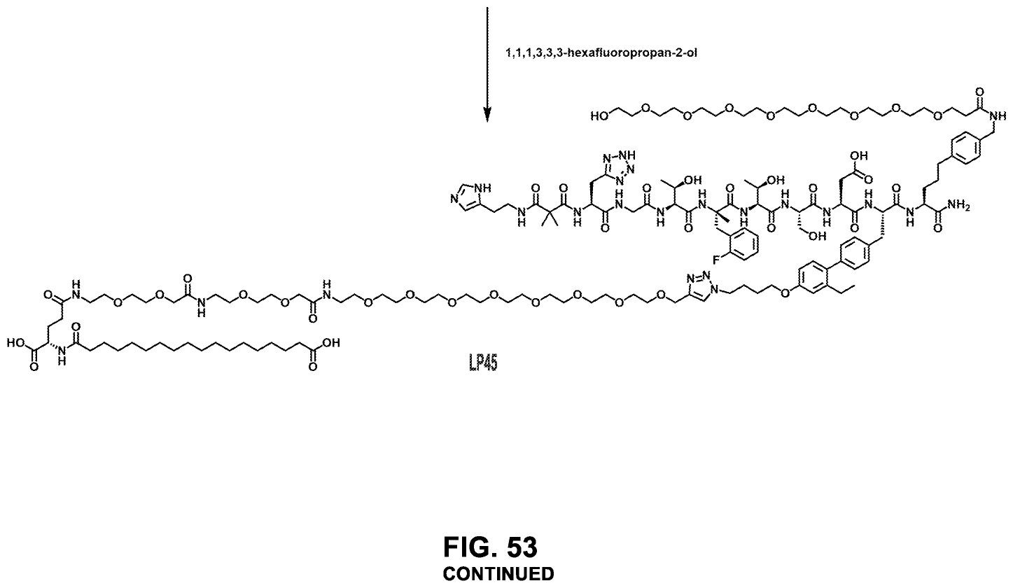

FIG. 53 depicts synthetic route of GLP-1R agonist Linker-payloads (LP45)

### Step 1: Preparation of (3S)-4-[[(1S)-1-[[4-[4-[4-[4-[2-[2-[2-[2-[2-[2-[2-[2-[[2-[2-[2-[[2-[2-[2-[[(4S)-5-tert-butoxy-4-[(18-tert-butoxy-18-oxo-octadecanoyl)amino]-5-oxo-pentanoyl]aminol ethoxy]ethoxy]acetyl]amino]ethoxy]ethoxy]acetyl]amino]ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[[(1 S)-1-carbamoyl-4-[4-[[3-[2-[2-[2-[2-[2-[2-[2-(2-hydroxyethoxy)ethoxy]ethoxy]ethoxy]ethoxy]ethoxy]ethoxyl ethoxy]propanoylamino]methyl]phenyl]butyl]amino]-2-oxo-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(1H-imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]-4-oxo-butanoic acid (LP45-2)

To a solution of LP29 (210 mg, 87.33 μmol, 1 eq.) in DMF (0.5 mL) were added DIPEA (22.57 mg, 174.66 μmol, 30.42 μL, 2 eq) and LP45-1 (168.93 mg, 174.66 μmol, 2 eq). The mixture was stirred at 25° C. for 1 hr. LCMS showed LP29 was consumed completely and one main peak with the desired mass was detected. The reaction mixture was partitioned in H₂O (20 mL) and extracted with EtOAc (10 mL×3). The organic phase was separated, washed with brine (10 mL), dried over Na₂SO₄, filtered and concentrated under reduced pressure to give a residue. The desired compound LP45-2 (200 mg, crude) was obtained as a yellow oil.

LCMS (ESI): RT=5.154 min, mass calcd. for C₁₅₅H₂₄₈FN₂₅O₄₇ 3231.78 [M+H]⁺, C₁₅₅H₂₅₁FN₂₅O₄₇ 1077.9 [M+3H]³⁺, found 1078.45 [M+3H]³⁺. LCMS method A: a MERCK, RP-18e 25-2 mm column, with a flow rate of 1.5 mL/min, eluting with a gradient of 5% to 95% acetonitrile containing 0.02% TFA (solvent B) and water containing 0.04% TFA (solvent A).

### Step 2: Preparation of 18-[[(1S)-4-[2-[2-[2-[2-[2-[2-[2-[2-[2-[2-[2-[2-[2-[2-[[1-[4-[4-[4-[(2S)-3-[[(1S)-1-carbamoyl-4-[4-[[3-[2-[2-[2-[2-[2-[2-[2-(2-hydroxyethoxy)ethoxy]ethoxy]ethoxyl ethoxy]ethoxy]ethoxy]ethoxy]propanoylamino]methyl]phenyl]butyl]amino]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(1H- imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]butyl]triazol-4-yl]methoxy]ethoxy]ethoxy]ethoxy]ethoxy]ethoxy]ethoxy]ethoxyl ethylamino]-2-oxo-ethoxy]ethoxy]ethylamino]-2-oxo-ethoxy]ethoxy]ethylamino]-1-carboxy-4-oxo-butyl]amino]-18-oxo-octadecanoic acid (LP45)

To a solution of LP45-2 (200 mg, 61.87 μmol, 1 eq) in 1,1,1,3,3,3-HEXAFLUORO-2-PROPANOL (2 mL). The mixture was stirred at 90° C. for 2 hr under microwave. LCMS showed LP45-2 was consumed completely and one main peak with desired mass was detected. LCMS (ESI): RT=3.575 min, mass calcd. for C₁₄₇H₂₃₃FN₂₅O₄₇ 3119.65 [M+H]⁺, C₁₄₇H₂₃₅FN₂₅O₄₇ 780.7 [M+4H]⁴⁺, found 781.0 [M+4H]⁴⁺. LCMS method A: a MERCK, RP-18e 25-2 mm column, with a flow rate of 1.5 mL/min, eluting with a gradient of 10% to 80% acetonitrile containing 0.02% TFA (solvent B) and water containing 0.04% TFA (solvent A). The reaction mixture was concentrated under vacuum to give crude. The residue was purified by prep-HPLC (column: YMC-Actus Triart C18 150*30 mm*5 μm; mobile phase: [water (0.1% TFA)-ACN]; B %: 36%-56%, 10.5 min) to obtain LP45 (18.03 mg, 5.75 μmol, 9.29% yield, 99.51% purity) as a white solid.

LCMS (ESI): RT=3.550 min, m/z calcd. for C₁₄₇H₂₃₃FN₂₅O₄₇ 3119.65 [M+H]⁺, C₁₄₇H₂₃₅FN₂₅O₄₇ 780.7 [M+4H]⁴⁺, found 781.0 [M+4H]⁴⁺. LC-MS method A: a MERCK, RP-18e 25-2 mm column, with a flow rate of 1.5 mL/min, eluting with a gradient of 10% to 80% acetonitrile containing 0.02% TFA (solvent B) and water containing 0.04% TFA (solvent A).

### Example 6. Preparative HPLC Purification of the Crude Peptidomimetics

The preparative HPLC was carried out on a Shimadzu LC-8a Liguid chromatograph. A solution of crude peptide dissolved in DMF or water was injected into a column and eluted with a linear gradient of ACN in water. Different methods were used. (See General Information). The desired product eluted were in fractions and the pure peptidomimetics were obtained as amorphous with powders by lyophilization of respective HPLC fractions. In general, after the prep-HPLC purification, the overall recovery was found to be in the range of 40˜50% yield.

Preparative HPLC method A: using FA condition (column: Xtimate C18 150*25 mm*5 μm; mobile phase: [water (0.225% FA)-ACN]; B %: 40%-70%, 7 min) to afford a pure product.

Preparative HPLC method B: using TFA condition (column: YMC-Exphere C18 10 μm 300*50 mm 12 nm; mobile phase: [water (0.1% TFA)-ACN]; B %: 15%-45%, 55 min) to afford a pure product.

Preparative HPLC method C: using neutral condition (column: Phenomenex Gemini-NX 150*30 mm*5 μm; mobile phase: [water (10 mM NH₄HCO₃)-ACN]; B %: 21%-51%, 11 min) to afford a pure product.

Preparative HPLC method D: using neutral condition (column: Waters Xbridge 150*255u; mobile phase: [water (10 mM NH₄HCO₃)-ACN]; B %: 20%-50%, 7 min) to afford a pure product.

Preparative HPLC method E: using FA condition (column: Phenomenex Luna C18 250*50 mm*10 um; mobile phase: [water (0.225% FA)-ACN]; B %: 55%-86%, 21 min) to afford a pure product.

### Example 7. Preparation of Antibody-Drug Conjugates

### 7.1 General Site-Specific Conjugation

This example demonstrates two methods for site-specific ATDC conjugation, generally, of a payload to an antibody comprising a Q-tag thereof. ATDCs 1-30 were prepared with LP1-LP11 and anti-GLP1R antibodies mAb1-mAb13 or control antibodies mAb1-mAb6 as summarized in Table 3 below. The ES-MS results and DAR values of the ADCs according to the disclosure are summarized in Table 3.

TABLE 3
Site-specific GLP1 Antibody-Drug Conjugates

| | | ADC | | | | ATDC |
|---|---|---|---|---|---|---|
| Target Ab | Ab # | LP # | Yield | LCMS DAR | MS (DAR2) | # |
| GLP1R | COMP mAb 1 | LP1 | 20% | 1.4 | 103081 | 1 |
| | (REGN5203) | | | | F(ab′)2 | |
| non-target | Isotype control | LP1 | 50% | 2.0 | 150044 | 2 |
| molecule | mAb 3 | | | | | |
| (Cont 1) | (REGN4100: Anti | | | | | |
| | ADRA2A (incl. N- | | | | | |
| | term TG | | | | | |
| | sequence: LLQGSG | | | | | |
| | (SEQ ID NO: 18))) | | | | | |
| GLP1R | COMP mAb 1 | LP2 | 35% | 1.5 | 103435 | 3 |
| | (REGN5203) | | | | F(ab′)2 | |
| non-target | Isotype control | LP2 | 70% | 2.0 | 150396 | 4 |
| molecule | mAb 3 | | | | | |
| (Cont 1) | (REGN4100: Anti | | | | | |
| | ADRA2A (incl. N- | | | | | |
| | terminal HC | | | | | |
| | Qtag:LLQGSG | | | | | |
| | (SEQ ID NO: 18))) | | | | | |
| GLP1R | COMP mAb 1 | LP3 | 40% | 1.4 | 151304 | 5 |
| | (REGN5203) | | | | | |
| non-target | Isotype control | LP3 | 30% | 2.0 | 150748 | 6 |
| molecule (Cont 1) | mAb 3 | | | | | |
| | (REGN4100: Anti | | | | | |
| | ADRA2A (incl. N- | | | | | |
| | term TG | | | | | |
| | sequence: LLQGSG | | | | | |
| | (SEQ ID NO: 18))) | | | | | |
| GLP1R | COMP mAb 1 | LP4 | 93% | 2.0 | 155254 | 7 |
| | (REGN5203) | | | | | |
| GLP1R | mAb 7 | LP4 | 91% | 2.0 | 149325 | 8 |
| | (REGN5204) | | | | | |
| GLP1R | mAb 8 | LP4 | 50% | 4.2 | 156816 | 9 |
| | (REGN5206: anti- | | | | (DAR4) | |
| | APLN incl. N- | | | | | |
| | terminal HC Qtag: | | | | | |
| | LLQGSG (SEQ ID | | | | | |
| | NO: 18)) | | | | | |
| GLP1R | mAb 9 | LP4 | 97% | 3.3 | 154870 | 10 |
| | (REGN5617) | | | | | |
| GLP1R | mAb 2 | LP4 | 99% | 2.4 | 155367 | 11 |
| | (REGN5619) | | | | | |
| non-target | control mAb 4 | LP4 | 40% | 2.0 | 151752 | 12 |
| molecule (Cont 2) | (REGN6497: anti- | | | | | |
| | Bet v 1 [Betula | | | | | |
| | pendula])) | | | | | |
| non-target | control mAb 5 | LP4 | 95% | 2.0 | 155027 | 13 |
| molecule (Cont 3) | (REGN7489: anti- | | | | | |
| | Bet v 1 [Betula | | | | | |
| | pendula]) | | | | | |
| non-target | control mAb 6 | LP4 | 95% | 2.2 | 155060 | 14 |
| molecule (Cont 4) | (REGN7490: anti- | | | | | |
| | Bet v 1 [Betula | | | | | |
| | pendula])) | | | | | |
| non-target | Isotype control | LP4 | 55% | 2.0 | 151789 | 15 |
| molecule (Cont 1) | mAb 3 | | | | | |
| | (REGN4100) | | | | | |
| GLP1R | COMP mAb 1 | LP6 | 30% | 1.1 | 150757 | 16 |
| | (REGN5203) | | | | | |
| GLP1R | mAb 10 | LP6 | 8% | 2.9 | 153054 | 17 |
| | (REGN5617) | | | | | |
| GLP1R | mAb 2 | LP6 | 77% | 2.5 | 153738 | 18 |
| | (REGN5619) | | | | | |
| non-target | control mAb 5 | LP6 | 28% | 2.0 | 153389 | 19 |
| molecule (Cont 3) | (REGN7489) | | | | | |
| GLP1R | mAb 2 | LP9 | 44% | 2.2 | 153413 | 20 |
| | (REGN5619) | | | | | |
| GLP1R | mAb 2 | LP11 | 72% | 1.2 | 152981 | 21 |
| | (REGN5619) | | | | | |
| GLP1R | mAb 6 | LP11 | 69% | 1.6 | 152994 | 22 |
| | (REGN9426) | | | | | |
| GLP1R | mAb 11 | LP11 | 64% | 1.5 | 151878 | 23 |
| | (REGN7989) | | | | | |
| GLP1R | mAb 3 | LP11 | 68% | 1.5 | 151876 | 24 |
| | (REGN7990) | | | | | |
| GLP1R | mAb 12 | LP11 | 55% | 1.6 | 153132 | 25 |
| | (REGN8069) | | | | | |
| GLP1R | mAb 5 | LP11 | 49% | 1.4 | 152661 | 26 |
| | (REGN9267) | | | | | |
| GLP1R | mAb 13 | LP11 | 46% | 1.7 | 152769 | 27 |
| | (REGN8071) | | | | | |
| GLP1R | mAb 4 | LP11 | 41% | 1.5 | 152735 | 28 |
| | (REGN8072) | | | | | |
| GLP1R | Isotype control | LP4 | 61% | 1.7 | 155264 | 31 |
| | mAb 3 | | | | | |
| | (REGN4100) | | | | | |
| GLP1R | mAb 14 | LP11 | 25% | 1.4 | 153549 | 32 |
| | (REGN7987) | | | | | |
| GLP1R | mAb 15 | LP11 | 53% | 1.3 | 153514 | 33 |
| | (REGN7988) | | | | | |
| GLP1R | mAb 16 | LP11 | 66% | 1.1 | 153096 | 34 |
| | (REGN8070) | | | | | |
| GLP1R | mAb 17 | LP11 | 47% | 1.3 | 152660 | 35 |
| | (REGN9268) | | | | | |
| GLP1R | mAb 18 | LP11 | 25% | 1.1 | 154279 | 36 |
| | (REGN9270) | | | | | |
| GLP1R | mAb 19 | LP11 | 62% | 0.9 | 154246 | 37 |
| | (REGN9278) | | | | | |
| GLP1R | mAb 20 | LP11 | 27% | 1.3 | 154623 | 38 |
| | (REGN9279) | | | | | |
| GLP1R | mAb 21 | LP11 | 12% | 1.0 | 154614 | 39 |
| | (REGN9280) | | | | | |
| GLP1R | mAb 6 | LP11 | 67% | 1.6 | 152994 | 40 |
| | (REGN9426) | | | | | |
| GLP1R | mAb 2 | LP11 | 82% | 1.4 | 152989 | 41 |
| | (REGN5619) | | | | | |
| GLP1R | mAb 3 | LP11 | 60% | 1.6 | 151882 | 42 |
| | (REGN7990) | | | | | |
| GLP1R | mAb 4 | LP11 | 60% | 1.7 | 152731 | 43 |
| | (REGN8072) | | | | | |
| GLP1R | mAb 5 | LP11 | 69% | 1.7 | 152660 | 44 |
| | (REGN9267) | | | | | |
| GLP1R | mAb 3 | LP11 | 73% | 1.7 | 25811 (LC- | 45 |
| | (REGN7990) | | | | DAR1) | |
| GLP1R | mAb | LP11 | 69% | 1.5 | 25759 (LC- | 46 |
| | 15(REGN7988) | | | | DAR1) | |
| GLP1R | mAb | LP11 | 86% | 1.3 | 26660 (LC- | 4 |
| | 16(REGN8070) | | | | DAR1) | |
| GLP1R | mAb | LP11 | 81% | 1.5 | 26007 (LC- | 48 |
| | 17(REGN9268) | | | | DAR1) | |
| GLP1R | mAb | LP11 | 86% | 1.0 | 26146 (LC- | 49 |
| | 19(REGN9278) | | | | DAR1) | |
| GLP1R | mAb 3 | LP11 | 78% | 1.7 | 151865 | 50 |
| | (REGN7990) | | | | | |
| GLP1R | mAb | LP11 | 63% | 1.7 | 152637 | 51 |
| | 17(REGN9268) | | | | | |
| GLP1R | mAb 3 | LP11 | n/a | 2.1 | 148977 | 52 |
| | (REGN7990) | | | | | |
| GLP1R | mAb | LP11 | n/a | 2.1 | 149754 | 53 |
| | 17(REGN9268) | | | | | |
| GLP1R | mAb 3 | LP11 | 81% | 2.2 | 148971 | 54 |
| | (REGN7990) | | | | | |
| GLP1R | mAb | LP11 | 60% | 2.1 | 149752 | 55 |
| | 17(REGN9268) | | | | | |
| GLP1R | mAb 3 | LP11 | 78% | 2.2 | 148978 | 56 |
| | (REGN7990) | | | | | |
| GLP1R | mAb | LP11 | 80% | 2.1 | 149753 | 57 |
| | 17(REGN9268) | | | | | |
| GLP1R | REGN15869 | LP11 | 80% | 2.0 | 148980 | |
| | | | | | (degly) | |
| non-target | Isotype control | LP11 | 65% | 1.7 | 151998 | 29 |
| molecule (Cont 5) | mAb 1 | | | | | |
| | (REGN7437: anti- | | | | | |
| | SCN9A) | | | | | |
| non-target | Isotype control | LP11 | 62% | 1.5 | 151995 | 30 |
| molecule (Cont 6) | mAb 2 | | | | | |
| | (REGN7438: anti- | | | | | |
| | SCN9A) | | | | | |
| non-target | Isotype control | LP11 | 80% | 1.7 | 151996 | 58 |
| molecule (Cont 6) | mAb 2 | | | | | |
| | (REGN7438: anti- | | | | | |
| | SCN9A) | | | | | |
| non-target | Isotype control | LP11 | 91% | 1.6 | 26410 (LC- | 59 |
| molecule (Cont 6) | mAb 2 | | | | DAR1) | |
| | (REGN7438: anti- | | | | | |
| | SCN9A) | | | | | |
| C. Difficile (Cont 7) | Isotype control | LP11 | 64% | 1.1 | n/a | 60 |
| | mAb 7 | | | | | |
| hANGPTL4 (Cont | Isotype control | LP11 | 52% | 1.3 | n/a | 61 |
| 8 | mAb 8 | | | | | |
| EGFRvIII (Cont 9) | Isotype control | LP11 | 51% | 1.6 | n/a | 62 |
| | mAb 9 | | | | | |
| | (REGN7438) | | | | | |

### 7.2 General Two-Step Conjugation Protocol

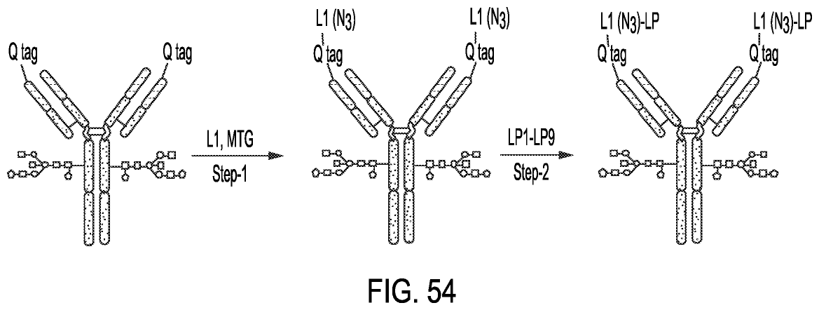

This method includes two step process shown in FIG. 54. The first step is microbial transglutaminase (MTG) mediated attachment of a First Linker (La), e.g., a small molecular amine, e.g., an azide-PEG3-amine, to the antibody, wherein an excess of the amine reagent is used to avoid potential cross-linking of antibody chains. The second step attaches the alkyne-linked payload linker payload (LP) to the Azido-tagged conjugate via a strain-promoted azide-alkyne cycloaddition (SPAAC).

The generic procedures are following.

### Step 1: Making a Site-Specific Azido-Functionalized Antibody Drug Conjugate Containing Two Azido Groups.

Anti-GLP1R human IgG antibody or isotype control antibody containing Q-tag was mixed with 100-200 molar equivalent of azido-PEG3-amine (L1, MW 218.26 g/mol). The resulting solution was mixed with transglutaminase (Zedira, Darmstadt, Germany, 1U MTG per mg of antibody) resulting in a final concentration of the antibody at 1-10 mg/mL. The reaction mixture was incubated at 25-37° C. for 4-72 hours with gently shaking while reaction was monitored by ESI-MS. Upon the completion, the excess amine and MTG were removed by size exclusion chromatography (SEC) or protein A column chromatography. The conjugate was characterized by UV-Vis, SEC and ESI-MS. The azido linkers attached antibody (Ab-N₃) resulting in a 402 Da mass increase for the DAR2 conjugate. Conjugate's monomer purity was >95%.

### Step 2: Making Site-Specific Conjugates in Table 1 Via [2+3] Click Reactions Between Azido-Functionalized Antibodies (Ab-N₃) and an Alkyne Containing Linker-Payload.

A site-specific antibody drug conjugate was prepared by incubating azido-functionalized antibody (Ab-N₃, 1-20 mg/mL) in PBS (pH7.4) with ≥3 molar equivalents of a linker-payload dissolved in an organic solvent, such as DMSO or DMA (10-20 mg/mL) to have the reaction mixture containing 5-15% organic solvent (v/v), at 25-37° C. for 1-48 hours while gently shaking. The reaction was monitored by ESI-MS. Upon completion, the excess amount of linker-payload and protein aggregates were removed by size exclusion chromatography (SEC). The purified conjugate was concentrated, sterile filtered and characterized by UV-Vis, SEC, HIC and ESI-MS. Conjugates monomer purity was >95%.

In a specific example, 36 mg anti-GLP1R mouse IgG antibody COMP mAb 1 containing a heavy chain N-term Q-tag was mixed with 150 molar equivalents of azido-PEG3-amine (L1, MW 218.26 g/mol). The resulting solution was mixed with microbial transglutaminase (1 U mTG per mg of antibody, Zedira, Darmstadt, Germany) resulting in a final concentration of the antibody at 4.0 mg/mL. The reaction mixture was incubated at 37° C. for 18 hours while gently shaking while monitored by ESI-MS. Upon the completion, the excess amine and mTG were removed by size exclusion chromatography (SEC). The concentrated site-specific antibody azido conjugate (2.9 mg/mL) in PBS (pH7.4) was mixed with 5 molar equivalents of linker-payload (LP4) in 10 mg/mL of DMSO. Additional DMSO was added to 10% total DMSO (v/v), and the solution was set at 25° C. for 22 hours with gently shaking. The reaction was monitored by ESI-MS. Upon completion, the excess amount of linker-payload and protein aggregates were removed by size exclusion chromatography (SEC). The purified conjugate was concentrated, sterile filtered and characterized by UV-Vis, SEC, HIC and ESI-MS. Conjugate monomer purity was 99.7%. The drug attached antibody resulting in a 5931 Da mass increase for the DAR2 conjugate.

### S7.3 General One-Step Conjugation Protocol

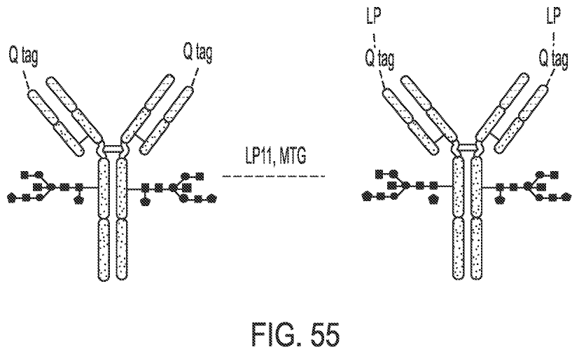

This method includes microbial transglutaminase (MTG) mediated attachment of Linker payload (LP) to the antibody, shown in FIG. 55.

The generic procedure is following.

Anti-GLP1R human IgG antibody or isotype control antibody containing Q-tag was mixed with 10-20 molar equivalent of linker payload (LP11, MW 1979.24 g/mol); Tris-HCl was added to elevate pH to around pH7.4. The resulting solution was mixed with transglutaminase (Zedira, Darmstadt, Germany, 1U MTG per mg of antibody) resulting in a final concentration of the antibody at 1-10 mg/mL. The reaction mixture was incubated at 25-37° C. for 4-72 hours with gently shaking while reaction was monitored by ESI-MS. Upon the completion, the excess amount of linker-payload, protein aggregates and MTG were removed by size exclusion chromatography (SEC). The purified conjugate was sterile filtered and characterized by UV-Vis, SEC, HIC and ESI-MS. Conjugates monomer purity was >95%.

In a specific example, 1.5 mg anti-GLP1R human IgG antibody mAb 6 containing a heavy chain N-term Q-tag was mixed with 15 molar equivalents of linker-payload (LP11) in 10 mg/mL of DMSO. Additional DMSO was added to a 10% total DMSO (v/v), followed by 1M Tris-HCl pH8 to bring the Tris concentration to 25 mM (pH ˜8). The resulting solution was mixed with transglutaminase (Zedira, Darmstadt, Germany, 1U MTG per mg of antibody) resulting in a final concentration of the antibody at 3.0 mg/mL. The solution was set at 37° C. for 23 hours with gently shaking. The reaction was monitored by ESI-MS. Upon completion, the excess amount of linker-payload, protein aggregates and MTG were removed by size exclusion chromatography (SEC). The purified conjugate was concentrated, sterile filtered and characterized by UV-Vis, SEC, HIC and ESI-MS. The drug attached antibody resulting in a 3924 Da mass increase for the DAR2 conjugate.

### Example 8. In Vitro Characterization of Payloads and Linker-Payloads

8.1 Human GLP1R cAMP Accumulation Assay (Cyclic AMP Determination)

The functional activity of the test compounds for agonizing GLP1R were evaluated using a cell-based assay. For the assay, HEK293 cells over-expressing full length human GLP-1R were utilized and the downstream cAMP accumulation was assessed as a measure of human GLP-1R stimulation. For the assay, compounds were 5-fold serially diluted in DMSO with a starting concentration of 1 μM in PP-384 microplates using an automated Bravo liquid handling platform. Diluted compounds were transferred to OptiPlates (100 nL/well) using an Echo liquid handler. HEK293/hGLP1R cells were thawed in 37° C. water-bath, washed 2 times with HBSS and re-suspended in assay buffer (HBSS+5 mM HEPES+500 μM IBMX+0.1% BSA). After the compounds were added, HEK293 cells were then seeded at 1×10⁵ cells/well (10 μL) into the 384 OptiPlates containing diluted compounds. After centrifugation, the assay plate was incubated at 23° C. for 30 min. The reaction was terminated by adding 10 μL of lysis buffer containing D2-cAMP and a cAMP-antibody from the cyclic AMP immunoassay kit (Cisbio, Cat #62AM4PEJ). Following a one-hour incubation, assay plates were read on an EnVision plate reader at 665/615 nm. The levels of cAMP per well were calculated using a standard curve generated by GraphPad Prism. Percent activity was calculated according to the formula (% Activity=100%*(cAMP level-LC)/(HC-LC)). EC₅₀ values were fitted from a four-parameter logistic equation over a 10-point response curve (GraphPad Prism).

As shown in Table 4, the test payloads and linker-payloads demonstrated cAMP activation with EC₅₀ values ranging from 39 μM to >11 nM, with most agonizing GLP1R with EC₅₀ values of <1 nM. The reference compound, GLP1, demonstrated cAMP activation with an EC₅₀ value of 28 pM.

TABLE 4
Activity of Payloads and Linker-Payloads in
the cAMP Accumulation Assay

| P# | cAMP EC₅₀ (nM) |
|---|---|
| GLP1 | 0.028 |
| P3 | 1.433 |
| P4 | 0.156 |
| P5 | 0.115 |
| P6 | 0.853 |
| P7 | 2.055 |
| P8 | 0.497 |
| P9 | 0.050 |
| P10 | 0.047 |
| P11 | 1.028 |
| P12 | 0.039 |
| P13 | 0.914 |
| P14-S | 0.727 |
| P15-R | 0.117 |
| P16-S | 0.544 |
| P17-R | 11.240 |
| P18 | 0.370 |
| P19 | 0.156 |
| P20 | 0.370 |
| P21 | 0.427 |
| P22 | 0.603 |
| P23 | 0.198 |
| P24 | 0.136 |
| LP5 | 0.044 |
| LP6 | 0.436 |
| LP7 | 0.378 |
| LP8 | 0.412 |
| LP9 | 0.078 |
| LP10 | 0.982 |
| LP15 | 0.649 |
| LP16 | 1.038 |
| LP11 | 0.318 |
| LP18 | 0.458 |
| LP19 | 0.558 |
| LP20 | 0.314 |
| LP22 | 2.706 |
| LP23 | 0.534 |

### 8.2 Plasma Stability

The plasma stability of payloads and linker payloads of the disclosure were measured. For the assay, pooled frozen plasma (human, mouse or monkey) was thawed in cold water or a 37° C. water bath prior to experiment. Plasma was centrifuged at 4000 rpm for 5 min if any clots were observed, they were subsequently removed. If required, the pH was adjusted to 7.4±0.1. For the preparation of compounds and positive controls, a 1 mM intermediate solution was prepared by diluting 10 μL of their stock solution with 90 μL DMSO; a 1 mM intermediate solution of the positive control (Propantheline) was prepared by diluting 10 μL of its stock solution with 90 μL ultrapure water. Subsequently, 100 μM dosing solution for each compound was prepared by diluting 20 μL of the intermediate solution (1 mM) with 180 μL 45% MeOH/H₂O. 98 μL of plasma was spiked with 2 μL of dosing solution (100 μM) to achieve the final concentration of 2 μM in duplicate. Samples were then incubated at 37° C. in a water bath. Four sets of time points were collected: (A) for the 2 hours test, the samples were collected at 0, 10, 30, 60 and 120 min; (B) for the 24 hours test the samples were collected at 0, 10, 60, 240 and 1440 min; (C) for the for 3 days test, the samples were collected at 0, 24, 48 and 72 hours (D) for the for 7 days test, the samples were collected at 0, 24, 48, 72, 96, 120, 144 and 168 hours. At each time point, 400 μL of stop solution (200 ng/mL tolbutamide plus 200 ng/mL labetalol in 50% ACN/MeOH) was added to precipitate protein and mixed thoroughly. Sample plates were then centrifuged at 4,000 rpm for 10 min. An aliquot of supernatant (50 μL) was transferred from each well and mixed with 100 μL ultrapure water. The samples were then shaken at 800 rpm for approximately 10 min before submitting for LC-MS/MS analysis.

As shown in Table 5A, most of the test payloads and linker-payloads were highly stable in human plasma, having T_{1/2}values of >48 hours in a one-day plasma assay and >14 days in a 7-day plasma assay; the reference compound, GLP1, is unstable in human plasma and demonstrated a T_{1/2} value of <10 minutes. As shown in Table 5B, the one linker payload of the disclosure (LP11) tested in monkey plasma had a T_{1/2} value of >49.4 hours. Two linker payloads of the disclosure (LP11 and LP23) that were tested in mouse plasma stability had T_{1/2} values of either >6 days in the 3 day assay or >4 hours in the 2 hour assay.

TABLE 5A
Human plasma stability of Payloads and Linker-Payloads

| | Human plasma stability | |
|---|---|---|
| | assay | Tested |
| P# | T½ (hr) | time* |
| P2 | 10.74 | B |
| P3 | >57.81 | A |
| P4 | >57.81 | A |
| P6 | >57.81 | A |
| P8 | >57.81 | A |
| P9 | >57.81 | A |
| P10 | 1.83 | B |
| P11 | >57.81 | A |
| P12 | >57.81 | A |
| P13 | 16.9 | A |
| P19 | >57.81 | A |
| P21 | >173.4 | C |
| P23 | 10.74 | B |
| P41 | >57.81 | A |
| LP11 | >173.4 (human) | C |
| LP11 | >49.4 (monkey) | C |
| LP11 | >173.4 (mouse) | C |
| LP23 | >4.8 hr (mouse) | B |
| | >4.8 hr (human) | |
| LP24 | >404.7 hr | D |
| LP25 | 284.8 hr | D |
| LP26 | >404.7 hr | D |
| LP28 | >173.4 | C |
| LP29 | >173.4 | C |
| LP34 | >173.4 | C |

*Tested for 1 day in A; 2 hrs in B; 3 days in C; 7 days in D.

TABLE 5B
Monkey and Mouse Plasma Stability of Linker-Payloads

| Species | P# | plasma stability T½ | Assay time |
|---|---|---|---|
| Monkey | LP11 | >49.4 hr | 3 days |
| Mouse | LP11 | >6 days | 3 days |
| | LP23 | >4 hr | 2 hr |

Table 5C, below, summarizes tested parameters for Linker-Payload LP₁₁ having the structure shown below disclosed as SEQ ID NO: 607.

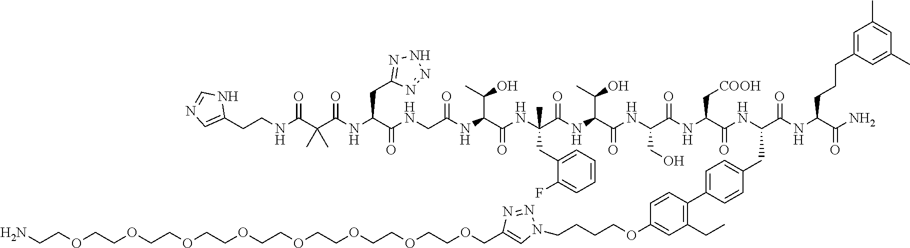

TABLE 5C
LP11 Characterization

| Assay | | Linker-Payload LP11 |
|---|---|---|
| GLP1R LUC EC₅₀ (nM) | | 0.016 nM |
| GLP2R, GIPR, or GCGR EC₅₀ (nM) | | Completely inactive |
| c-AMP EC₅₀ | | 0.318 nM |
| Plasma Stability | Mouse T½ | >7 days |
| | Monkey T_{1/2} | >2 days |
| | Human T½ | >7 days |
| Human Liver | T_{1/2} | >145 min |
| microsome | CL_{int(mic)} | <9.6 μL/min/mg |
| | CLᵢₙₜ₍ₗᵢᵥₑᵣ₎ | <8.6 mL/min/kg |

| | |
|---|---|
| Water solubility | 60 mM |
| Protein binding | 97.97% |
| hERG IC₅₀ | >100 uM |
| Ames (TA98 and TA100) | Negative for mutagenicity |
| in the presence and absence of S9 | |

8.3 hERG Assay

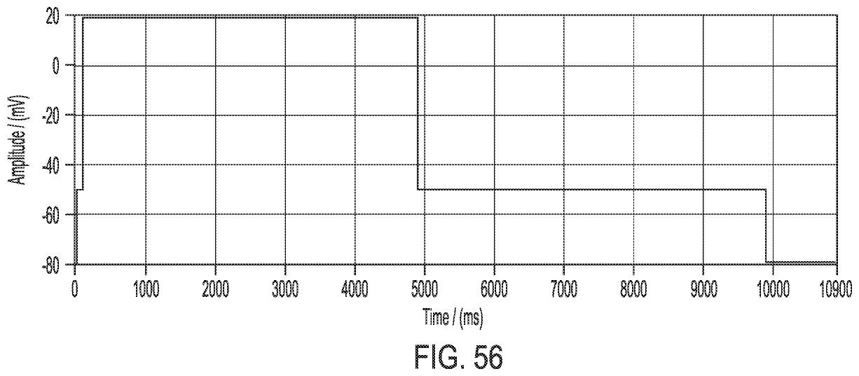

To determine if compounds of the disclosure had impacts on hERG potassium channels activity, cell based assay was performed. For the assay, CHO cells stably expressing hERG potassium channels were plated and cultured for at least 2 days in a humidified and air-controlled (5% CO₂) incubator at 37° C. prior to use in electrophysiological experiments. Cells were then harvested using TrypLE and resuspended in physiological solution (10 mM HEPES, 80 mM NaCl, 4 mM KCl, 2 mM CaCl₂, 1 mM MgCl₂, 5 mM Glucose, 60 mM NM DG, pH 7.4). Test compounds were dissolved in water to obtain stock solutions for different test concentrations. Stock solutions were further diluted into external electrophysiological recording solution (10 mM HEPES, 140 mM NaCl, 4 mM KCl, 2 mM CaCl₂, 1 mM MgCl₂, 5 mM Glucose, pH 7.4) to achieve final concentrations for testing. Recordings were performed at room temperature using a Nanion SyncroPatch 384PE, a 384-well automated patch-clamp platform (Internal recording solution: 10 mM HEPES, 10 mM NaCl, 10 mM KCl, 110 mM KF, 10 mM EGTA, pH 7.2). A schematic of the voltage command protocol used for electrophysiological recordings is shown in FIG. 56. One 40 μL addition of the vehicle was applied, followed by a 300s baseline recording period. Then 40 μL doses of compounds were added at each concentration with an exposure time of no less than 300s. Recordings were required to pass quality control over the duration of the recording or the well was abandoned, and the compound was retested, all automatically set by PatchControl. Five concentrations (0.3 μM, 3 μM, 10.00 μM, 30.00 μM and 100.00 μM) were tested for each compound as well as a positive control (Amitriptyline). A minimum of 2 replicates per concentration were obtained. Data analysis was carried out using DataControl, Excel 2013 (Microsoft) and GraphPad Prism 5.0. Curve-fitting and IC₅₀ value calculations were performed by GraphPad Prism 5.0. If the inhibition obtained at the lowest concentration tested was over 50%, or at the highest concentration tested was less than 50%, the IC₅₀ value was reported as less than lowest concentration, or higher than highest concentration, respectively. The IC₅₀ values of the test compounds on whole cell hERG currents were summarized in Table 6.

TABLE 6
IC₅₀ of the Test Compounds on Whole Cell hERG Currents

| Compound ID | IC₅₀ (μM) | HillSlope | N |
|---|---|---|---|
| Amitriptyline | 2.90 | 1.25 | 3 |
| LP10 | >100.00 | — | 2 |
| LP11 | >100.00 | — | 2 |

### 8.4 Ames Assay

The objective of this Mini-Ames study was to evaluate the test article LP11, for its ability to induce reverse mutations in the genome of strains of Salmonella typhimurium TA98 and TA100 in the presence and absence of metabolic activation (S9 mixture).

The Mini-Ames assay was conducted for the test article in the presence and absence of the S9 mixture, concurrently with the negative/solvent control (DMSO) using six wells and positive controls (2 μg/well TA98 alone, 0.2 μg/well TA100 alone or 0.4 μg/well TA98+TA100 in the presence of S9) using three wells. The tested dose levels for the test article, in the presence and absence of S9 mix, were 1000, 400, 160, 64, 25, 10, 4, and 1.5 μg per well, with each dose tested in triplicate. The study was conducted using fresh cultures of the bacterial strains and fresh test article formulations.

For test article LP11, no obvious cytotoxicity was observed at any dose level with or without S9 mix in any tester strain. No obvious precipitates (by naked eye after the incubation period) were observed at any dose level with or without S9 mix in any tester strain.

LP11 did not induce more than 2.0-fold increase in the two strains TA98 or TA100 in the mean number of revertant colonies at any dose level relative to the concurrent negative/solvent control, either in the presence or absence of S9 mix. No dose response was observed either.

For both tester strains used in this study, the mean number of revertant colonies observed for the negative/solvent control was comparable to the laboratory historical negative control data. All positive controls induced the expected increase of greater than three-fold in the mean number of revertant colonies, in the presence and absence of S9 mix, when compared to the concurrent negative/solvent control, thereby confirming the responsiveness of the strains.

The genotypes of all the tester strains used in this assay were confirmed. It was concluded that this Mini-Ames study was valid and LP11 was negative for mutagenicity under the conditions of this study.

### 8.5 In Vitro ADME

To assess the in vitro ADME properties of the LP, microsomal stability assay was performed to determine intrinsic clearance, and plasma protein binding assay was performed to understand the distribution potential. The results are listed in Tables 7 and 8 below.

TABLE 7
Liver Microsome Stability

| Sample | T_{1/2} | CL_{int(mic)} | CLᵢₙₜ₍ₗᵢᵥₑᵣ₎ |
|---|---|---|---|
| Name | (min) | (μL/min/mg) | (mL/min/kg) |
| LP11 | >145 | <9.6 | <8.6 |
| Testosterone | 16.2 | 85.7 | 77.2 |

An assay was performed to determine the plasma protein binding of compounds of the disclosure. For the assay, on the day of experiment, the plasma was thawed in cold water and centrifuged at 3220 rpm for 5 minutes to remove any clots. The pH value of the resulting plasma was checked

The 96-well equilibrium dialysis device (Cat #1006) and HTD 96 a/b cellulose membrane strips with molecular mass cutoff of 12-14 kDa (Cat #1101) were obtained from HTDialysis LLC, Gales Ferry, CT) and the dialysis device was assembled following the manufacturer's instructions. (<http://www.htdialysis.com/>).

For the dialysis, the test compound and control compound were dissolved in DMSO to achieve 10 mM stock solutions. DMSO working solutions were prepared at 400 μM. To prepare the loading matrix, compound working solutions (5 μL) were added in a 1:200 ratio to blank matrix (995 μL) and mixed thoroughly. To prepare the time zero (TO) samples to be used for recovery determination, 50 μL aliquots of loading matrix were transferred in triplicate to the sample collection plate. The samples were immediately matched with opposite blank buffer to obtain a final volume of 100 μL of 1:1 matrix/dialysis buffer (v/v) in each well. 500 μL of stop solution were added to these TO samples. This was then stored at 2-8° C. pending further processes along with other post-dialysis samples. To load the dialysis device, an aliquot of 150 μL of the loading matrix was transferred to the donor side of each dialysis well in triplicate, and 150 μL of the dialysis buffer was loaded to the receiver side of the well. The dialysis plate was placed in a humidified incubator at 37° C. with 5% CO₂ on a shaking platform that rotated slowly (about 100 rpm) for 4 hours. At the end of the dialysis, aliquots of 50 μL of samples were taken from both the buffer side and the matrix side of the dialysis device. These samples were transferred into new 96-well plates. Each sample was mixed with an equal volume of opposite blank matrix (buffer or matrix) to reach a final volume of 100 μL of 1:1 matrix/dialysis buffer (v/v) in each well. All samples were further processed by adding 500 μL of stop solution containing internal standards. The mixture was vortexed and centrifuged at 4000 rpm for about 20 minutes. An aliquot of 100 μL of supernatant of all the samples was then removed for LC-MS/MS analysis. The single blank samples were prepared by transferring 50 μL of blank matrix to a 96 well plate and adding 50 μL of blank PBS buffer to each well. The blank plasma must match the species of plasma used in the plasma side of the well. Then the matrix-matched samples were further processed by adding 500 μL of stop solution containing internal standards, following the same sample processing method as the dialysis samples.

The percent unbound, percent bound, and percent recovery values were calculated using the following equations:

$$\begin{matrix}\% Unbound - 100 \times \frac{[ F ]}{[ T ]} \\ \% Bound - 100 \times ( 1 - \frac{[ F ]}{[ T ]} ) \\ \% Recovery - 100 \times ( \frac{[ F ] + [ T ]}{[ {\overline{T}}_{0} ]}\end{matrix} ,$$

where [F] is the analyte concentration or peak area ratio of analyte/internal standard on the buffer (receiver) side of the membrane, [T] is the analyte concentration or peak area ratio of analyte/internal standard on the matrix (donor) side of the membrane, and [T0] is the analyte concentration or the peak area ratio of analyte/internal standard in the loading matrix sample at time zero. Table 8, below, summarizes the plasma protein binding assay results.

TABLE 8
Plasma Protein Binding Assay Results

| | | | % | | | % |
|---|---|---|---|---|---|---|
| Compound | Species/ | | Unbound | | | Recovery |
| ID | Matrix | % Unbound | SD | % Bound | % Recovery | SD |
| Warfarin | Human | 1.25 | 0.2 | 98.75 | 98.3 | 3.0 |
| | Plasma | | | | | |
| LP11 | Human | 2.03 | 0.4 | 97.97 | 89.2 | 1.7 |
| | Plasma | | | | | |

### Example 9. Activities of GLP1R Peptidomimetic Payloads and Linker-Payloads Against GPCR Family Members

To test the activity of GLP1R agonist payloads and GLP1R agonist linker-payloads (LPs) in vitro, a cell-based cAMP responsive luciferase reporter assay was developed. Human embryonic kidney cells (HEK293) expressing a cyclic AMP response element (CRE)-luciferase reporter were generated that express either Myc-tagged full length human GLP1R (amino acids 1 to 463 of accession number NP_002053); referred to as HEK293/Myc-hGLP1R/Cre-Luc cell line), full length human gastric inhibitory polypeptide receptor (GIPR) (amino acids 1 to 466 of accession number NP_000155.1; referred to as HEK293/Myc-hGIPR/Cre-Luc cell line), full length human glucagon-like peptide 2 receptor (GLP2R) (amino acids 1 to 553 of accession number NP_004237; referred to as HEK293/Myc-hGLP2R/Cre-Luc cell line) or full length human glucagon receptor (GCGR) (amino acids 1 to 477 of accession number NP_000151.1; referred to as HEK293/Myc-hGCGR/Cre-Luc cell line) using standard methods for the generation of stable cell lines. Cell surface expression of the receptors was confirmed by flow cytometry, using an antibody recognizing the Myc tag.

For the bioassay, cells were plated at a density of 20,000 cells/well in 80 μL of Opti-MEM supplemented with 0.1% FBS in a 96-well clear bottom white plates (Corning, #356693). Cells were incubated overnight at 37° C. in 5% CO₂. Test reagents, including payload, a linker payload, and positive control ligands [human GLP1 (R&D Systems, #5374), human GIP (R&D Systems, #2084), human GCG (R&D Systems, #6927), or human GLP2 (R&D Systems, #2258)], were serially diluted in Opti-MEM with 0.1% FBS and were then added to the cells at 10 μL/well for each concentration tested. Plates were incubated for 5.5 h at 37° C. in 5% CO₂. For end-point measurement, 100 μL/well of One-Glo reagent (Promega, #E6051) was added and plates were kept at room temperature for 5-10 minutes. Luciferase activity was measured on Envision Multilabel Plate Reader (Perkin Elmer) in Luminescent mode. The relative light units (RLU) values were obtained and the results were analyzed using nonlinear regression with GraphPad Prism software (GraphPad).

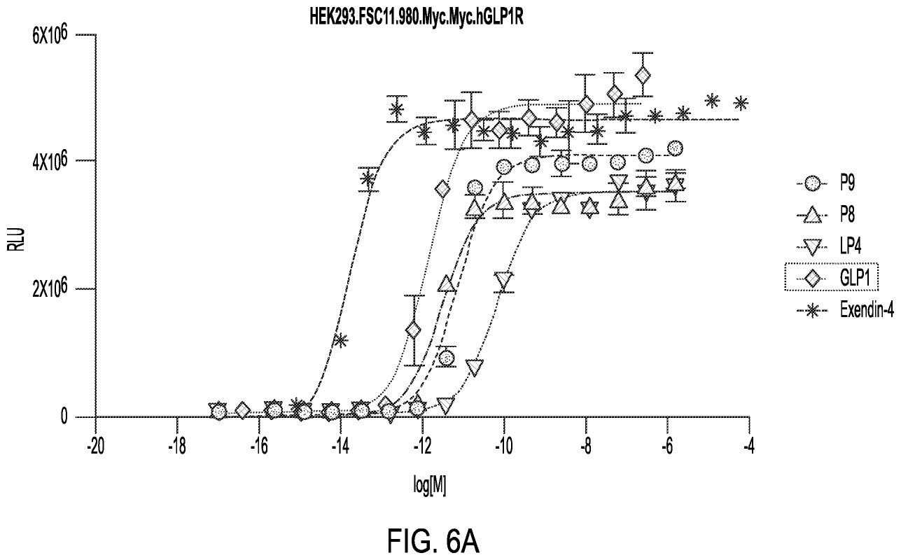

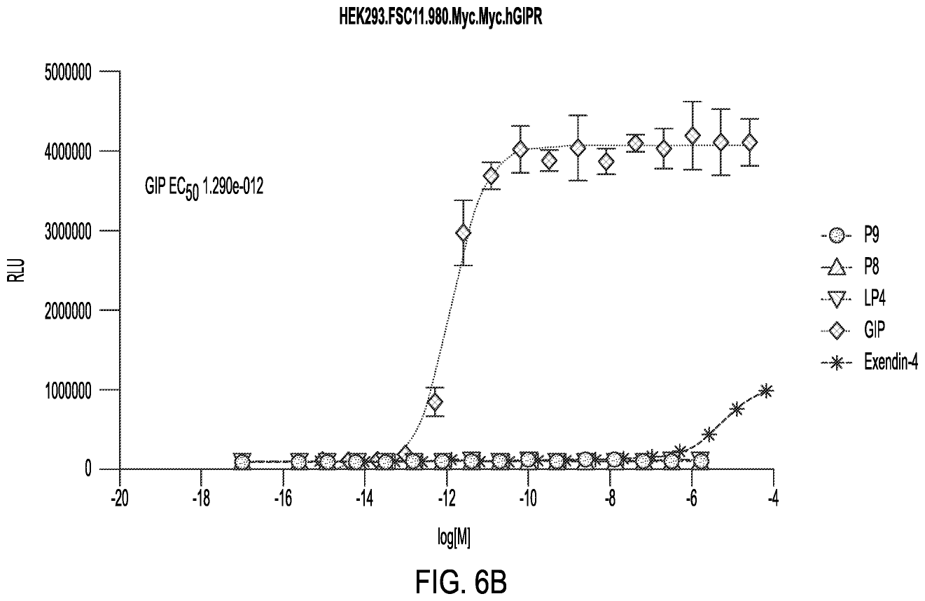

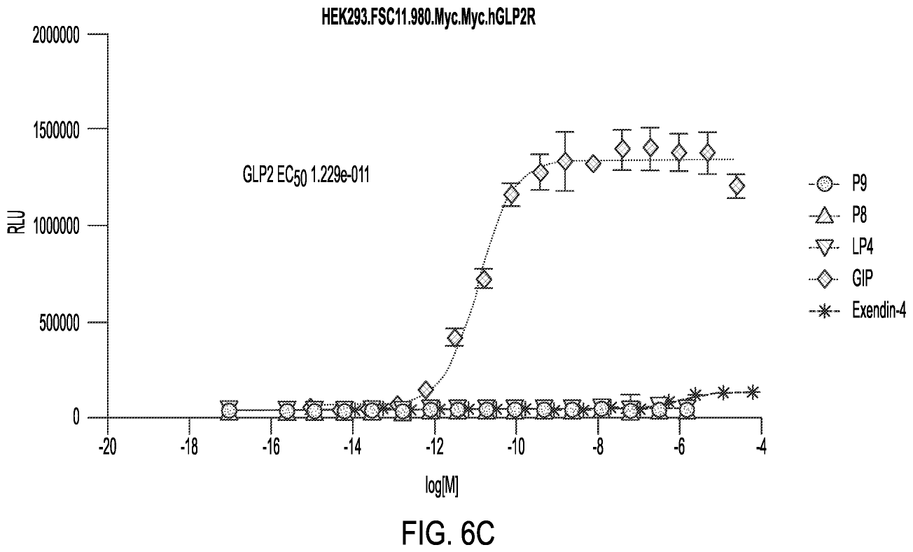

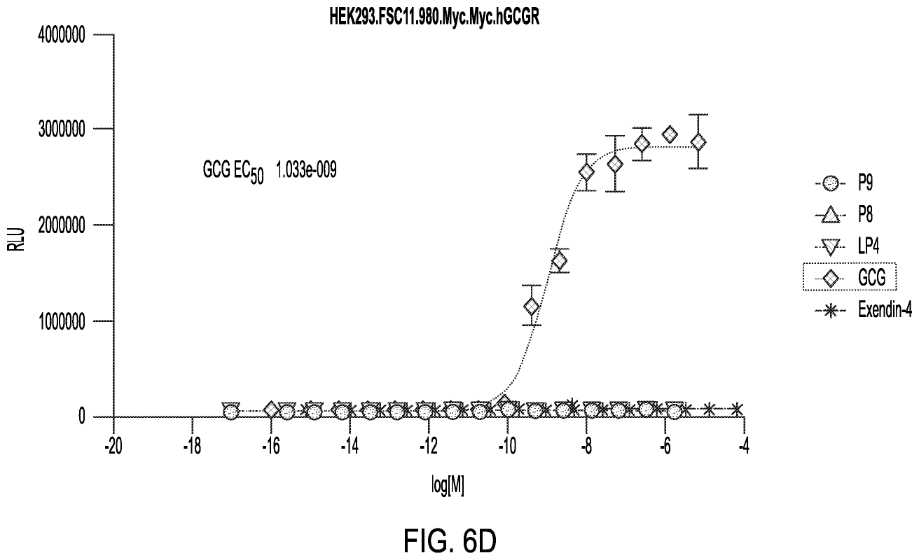

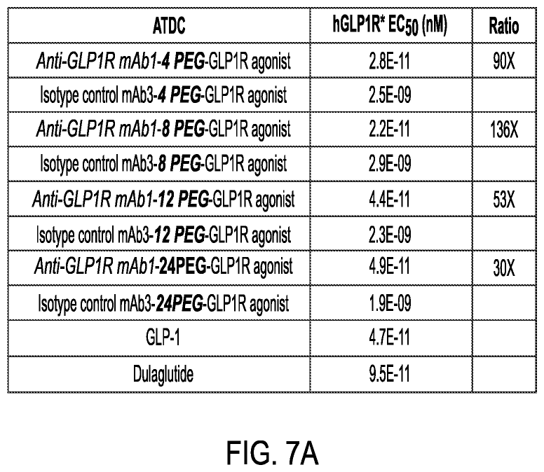

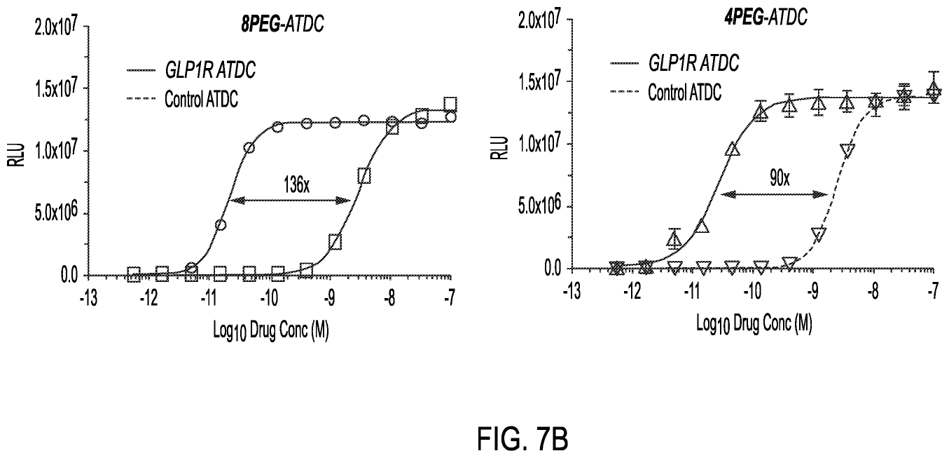

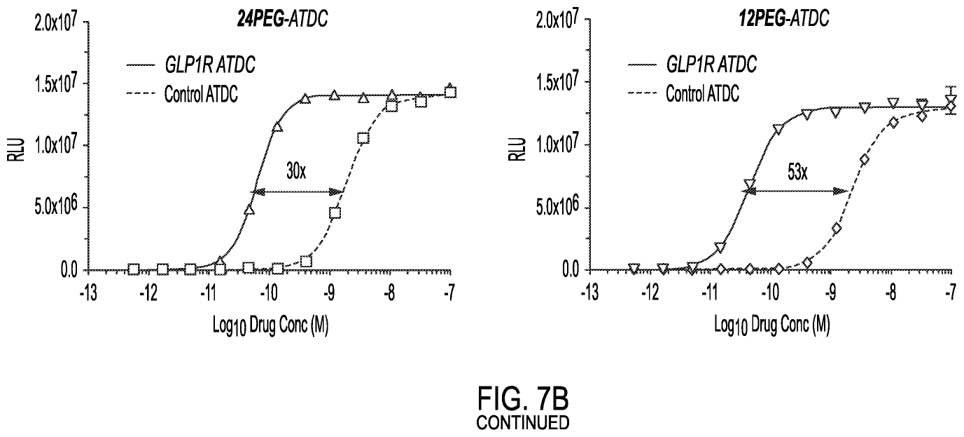

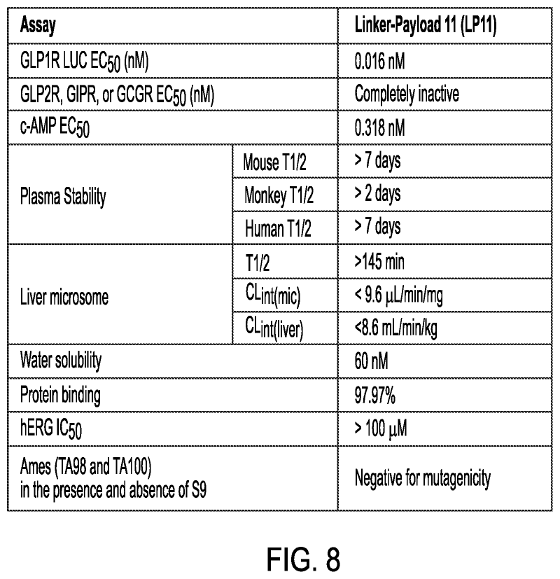

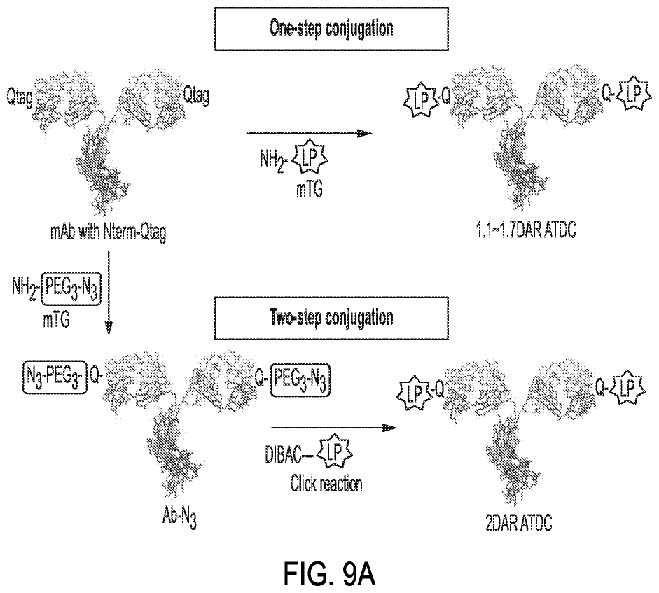

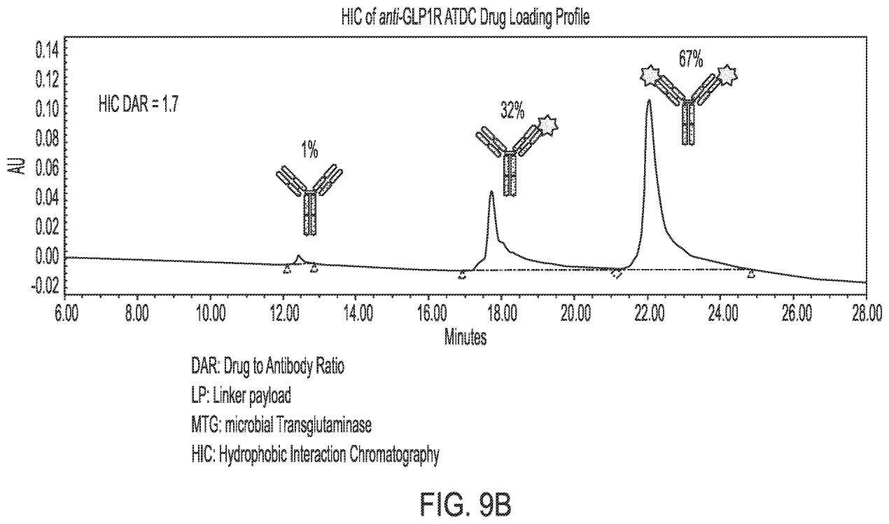

As shown in Table 9 and FIGS. 6A-6D, the payloads and linker-payload demonstrated activation in the HEK293/Myc-hGLP1R/Cre-Luc cell line with EC₅₀ values ranging from 3.32 μM to 71.5 μM. The payloads and linker payload did not demonstrate an measurable activity in the other cell lines evaluated. All of the positive control reference ligands (human GLP1, GIP, GLP2, and GCG) activated individual cell lines as expected.

TABLE 9
CRE-Dependent Reporter Activity by GLP1R payload and linker-
payload Agonists in GLP1R, GIPR, GLP2R and GCGR cell lines

| | HEK293/ | HEK293/ | HEK293/ | HEK293/ |
|---|---|---|---|---|
| | Myc- | Myc- | Myc- | Myc- |
| | hGLP1R/ | hGIPR/ | hGLP2R/ | hGCGR/ |
| | Cre-Luc | Cre-Luc | Cre-Luc | Cre-Luc |
| Compound | EC₅₀ (M) | EC₅₀ (M) | EC₅₀ (M) | EC₅₀ (M) |
| P9 | 3.32E−12 | N/A | N/A | N/A |
| P8 | 7.60E−12 | N/A | N/A | N/A |
| LP4 | 7.15E−11 | N/A | N/A | N/A |
| LP11 | 1.6E−11 | N/A | N/A | N/A |
| Reference* | 1.46E−12 | 1.29E−12 | 1.23E−11 | 1.03E−9 |

N/A = Not Active
Reference for GLP1R assay = GLP1
Reference for GIPR assay = GIP
Reference for GLP2R assay = GLP2
Reference for GCGR assay = GCG

### Example 10. In Vitro Plasma Stability

To determine the plasma stability of ATDC anti-GLP1R mAB2-LP11 bearing GLP1R agonist P8, the ATDCs was incubated in vitro with the plasma from different species and the drug to antibody ratio (DAR) was evaluated. Anti-GLP1R mAB2 is a biotinylated anti-Fc antibody.

The ATDC solution was spiked into pooled C57BL/6 mouse, cynomolgus monkey (Cyno), or IgG depleted human plasma (BiolVT) to a final concentration of 50 μg/mL, and subsequently incubated at 37° C. on ThermoMixer C (Eppendorf, Cat #2231000574). A 100-μL aliquot was removed at times 0, 1, 2, 3 and 7 days and then immediately stored frozen at −80° C. until analysis.

For DAR analysis, the ATDC was purified from plasma samples by immunoaffinity capture using a DynaMag-2 magnetic rack (Life Technologies, Cat #12321D). First, biotinylated anti-human Fc antibody (Regeneron generated reagent) was immobilized on Dynabeads M280 streptavidin beads (Invitrogen, Cat #60210). Each plasma sample containing the ATDC was mixed at 950 rpm with 0.5 mg of the beads at room temperature for 2 hours with gentle shaking. The beads were then washed three times with 500 μL of HBS-EP pH 7.4 buffer (GE Healthcare, Cat #BR100188), once with 500 μL water and once with 500 μL of 10% acetonitrile (VWR Chemicals, Cat #BDH83640.100E) in water. Following the washes, the ATDC was eluted by incubating the beads with 70 μL of 1% formic acid in 30:70 acetonitrile:water (v/v) for 15 minutes at room temperature. Fifty μL eluted samples were further reduced by adding 50 μL 10 mM TCEP (Sigma, Cat 646547-10X1 ML) and incubated at 37° C. for 20 min in ThermoMixer C.

The reduced ATDC samples were then injected onto a 0.3×50 mm 1.7 μm BEH300 C4 column (Waters, Cat #186009260) for separation and detected by Synapt G2-Si Mass Spectrometer (Waters). The flow rate used was 10 μL/min (mobile phase A: 0.1% formic acid in water; mobile phase B: 0.1% formic acid in acetonitrile). The HPLC gradient eluted ATDC between 3.5-6.5 minutes corresponding to 25-40% of mobile phase B. The acquired spectra were deconvoluted using MaxEnt1 software (Waters) with the following parameters: Mass range: 20-60 kDa; m/z range: 800-2500 Da; Resolution: 1.0 Da/channel; Width at half height: 0.8 Da; Minimum intensity ratios: 33%; Iteration max: 15. DAR was calculated based on peak intensity corresponding to individual DAR species in the deconvoluted spectra.

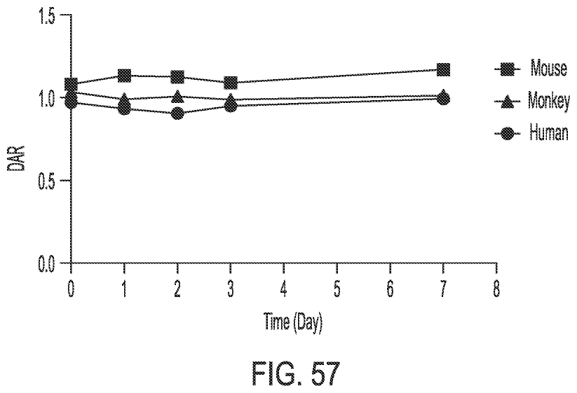

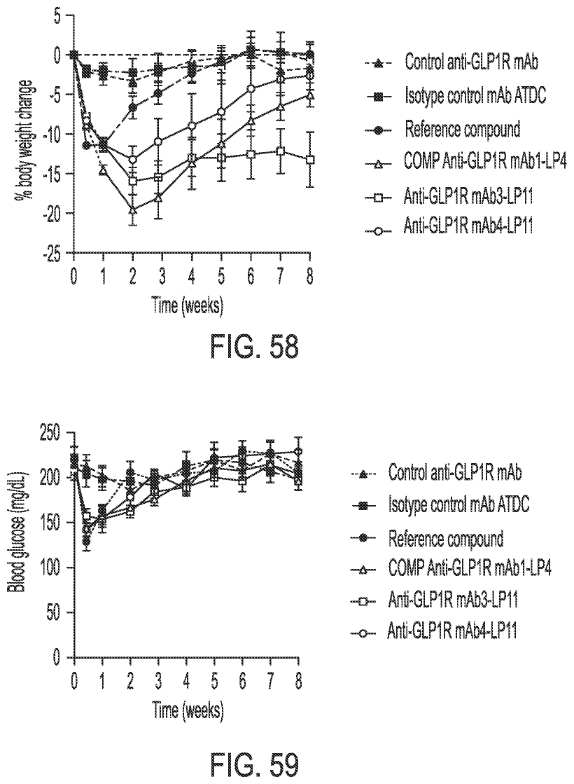

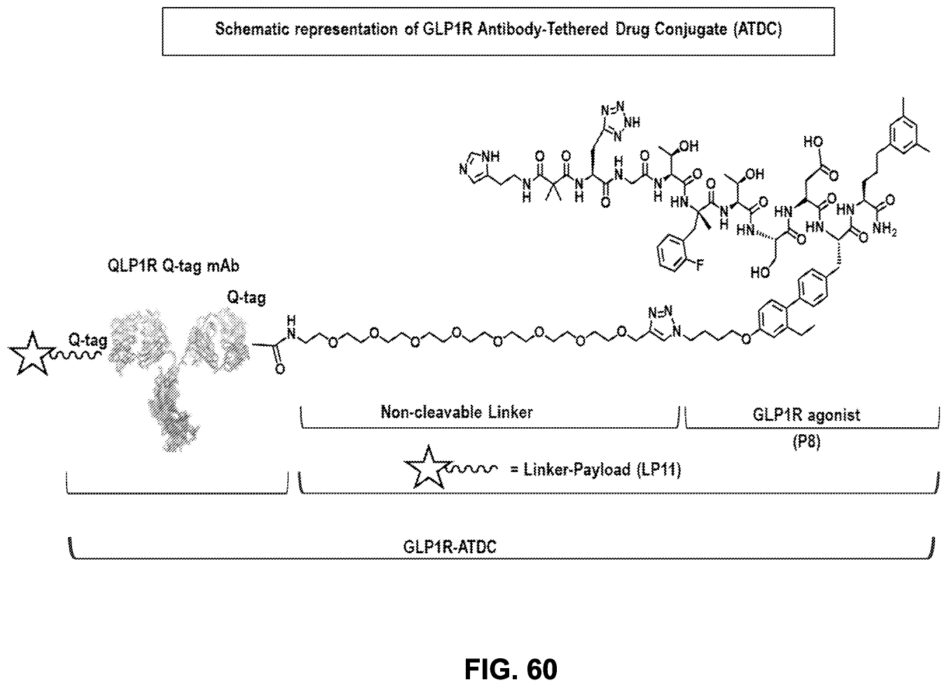

No significant change in DAR was observed for anti-GLP1R mAb2-LP11 after a 7-day incubation in mouse, cynomolgus monkey or IgG depleted human plasma. The results are presented in Table 10 and FIG. 57.

TABLE 10
In vitro stability of anti-GLP1R mAB2-LP11 over a 7-day, 37° C.
incubation in mouse, monkey and human plasma

| | DAR in | DAR in | DAR in |
|---|---|---|---|
| Time | Mouse | Monkey | Human |
| (Days) | Plasma | Plasma | Plasma |
| 0 | 1.1 | 1.0 | 0.97 |
| 1 | 1.1 | 0.99 | 0.93 |
| 2 | 1.1 | 1.0 | 0.90 |
| 3 | 1.1 | 0.99 | 0.95 |
| 7 | 1.2 | 1.0 | 0.99 |

### Example 11. Luciferase Reporter Assay

To test the activity of GLP1R agonist payloads, GLP1R agonist linker-payloads (LPs), and anti-GLP1R antibody tethered drug conjugates (ATDCs) of the disclosure, a cell-based cAMP responsive luciferase reporter assay was developed. To generate the assay cell line, the firefly luciferase gene was placed under the control of a cAMP response element (CRE) located upstream of a minimal promoter and transfected into HEK293 cells and referred to herein as HEK293/CRE-Luc cells. HEK293/CRE-Luc cells were then engineered to express full-length human GLP1R (amino acids 1 to 463 of accession number NP_002053) and are referred to herein as HEK293/CRE-Luc/hGLP1R cells.

For the assays, cells were seeded into 96 well plates at 10,000 or 20,000 cells/well in assay media (Optimem, 0.1% BSA, 100 units/ml Penicillin, 100 ug/ml Streptomycin, 292 μg/ml L-glutamine) and incubated overnight. Three-fold serial dilutions of free payloads or LPs were prepared in 100% DMSO, transferred to fresh assay media, and added to the cells at a final constant DMSO concentration of 0.2%. The last well in the plate served as a blank control containing only the assay media and 0.2% DMSO (untreated well) and was plotted as a continuation of the 3-fold serial dilution. Four to six hours later, luciferase activity was determined after the addition of One-Glo™ reagent (Promega, Cat #E6130) to each well. Relative light units (RLUs) were measured on an Envision luminometer (PerkinElmer) and EC₅₀ values were determined using a four-parameter logistic equation over a 12-point dose response curve (GraphPad Prism). The signal to noise (S/N) was determined by taking the ratio of the highest RLU on the dose response curve to the RLU in the untreated wells. EC₅₀ and S/N values are summarized in Table 11. Data was generated across several experiments (A, B, and D) with P8 serving as a reference standard in each experiment to calculate the payload relative potency [(P8 EC₅₀/payload EC₅₀)*100] and relative S/N [(payload SN/P8 SN)*100].

As shown in Table 11, payload and linker-payload EC₅₀ values ranged from 3.97 μM to 1.95 nM and relative potency (% P8) ranged from 0.3% to 133.8% in HEK293/CRE-Luc/hGLP1R cells. Most tested payloads reached similar max activity with relative S/N values (% P8) ranging from 68.6% to 116.27%. The GLP1 ligand was also included for reference and increased CRE-dependent luciferase activity in HEK293/CRE-Luc/hGLP1R cells with an EC₅₀ of 14.3 μM, relative potency of 37.2%, and relative S/N of 96.8%. All tested agonists had minimal impact on luciferase activity in the absence of hGLP1R expression (HEK293/CRE-Luc cells), with S/N values≤1.7 and EC₅₀ values>200.0 nM.

TABLE 11
CRE-Dependent Reporter Activity by GLP1R payload and linker-
payload Agonists in HEK293/CRE-Luc/hGLP1R cells

| | HEK293/CRE-LUC/hGLP1R | | | | | | |
|---|---|---|---|---|---|---|---|
| | | Relative | | Relative | | | |
| | | Potency | | Signal:Noise | HEK293/CRE-LUC | | |
| Molecule | EC₅₀ | (% P8 EC₅₀) | S/N | (% P8) | EC₅₀ | S/N | Experiment |
| P9 | 3.97E−12 | 133.8 | 75.2 | 114.3 | >2E−07 | 1.3 | A |
| P23 | 5.15E−12 | 103.0 | 57.1 | 86.9 | >2E−07 | 1.6 | A |
| P8 | 5.31E−12 | 100.0 | 65.8 | 100.0 | >2E−07 | 1.5 | A |
| P12 | 1.14E−11 | 46.5 | 76.4 | 116.2 | >2E−07 | 1.2 | A |
| P15-R | 1.32E−11 | 40.3 | 67.8 | 103.1 | >2E−07 | 1.1 | A |
| P10 | 1.40E−11 | 38.0 | 45.1 | 68.6 | >2E−07 | 1.2 | A |
| GLP1 | 1.43E−11 | 37.2 | 63.6 | 96.8 | >2E−07 | 1.3 | A |
| LP11 | 1.61E−11 | 33.1 | 74.5 | 113.3 | >2E−07 | 1.3 | A |
| P8 | 2.0E−11 | 100.0 | 327.9 | 100.0 | NT | NT | B |
| P8 | 2.73E−11 | 100.0 | 235.9 | 100.0 | NT | NT | D |
| P4 | 8.37E−11 | 6.3 | 66.9 | 101.8 | >2E−07 | 1.6 | A |
| LP4 | 1.27E−10 | 4.2 | 63.8 | 97.1 | >2E−07 | 1.7 | A |
| P13 | 3.57E−10 | 1.5 | 69.5 | 105.6 | >2E−07 | 1.2 | A |
| P6 | 6.02E−10 | 0.9 | 60.7 | 92.3 | >2E−07 | 1.0 | A |
| P11 | 6.85E−10 | 0.8 | 54.8 | 83.4 | >2E−07 | 1.1 | A |
| P21 | 7.38E−10 | 0.7 | 58.8 | 89.5 | >2E−07 | 1.3 | A |
| P3 | 1.95E−09 | 0.3 | 46.3 | 70.3 | >2E−07 | 1.2 | A |

NT = not tested
> = EC₅₀ values could not be determined with accuracy because the binding did not reach saturation within the tested antibody concentration range. EC₅₀ is reported as greater than the highest tested concentration

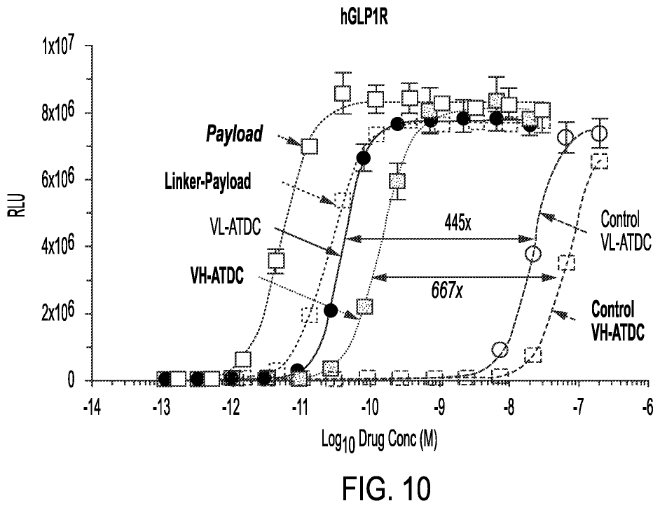

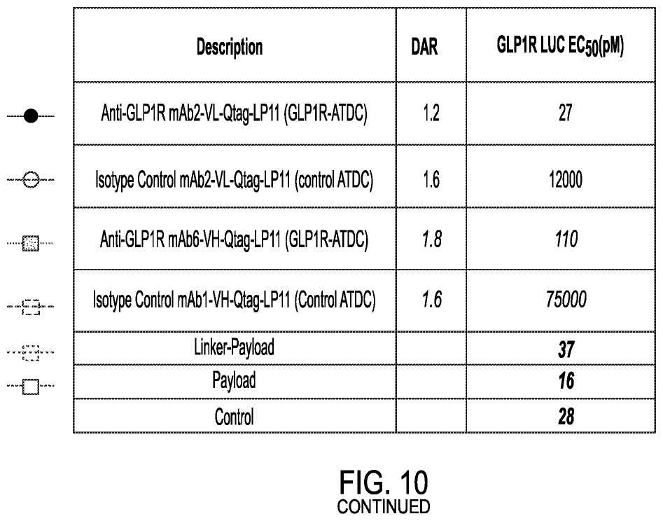

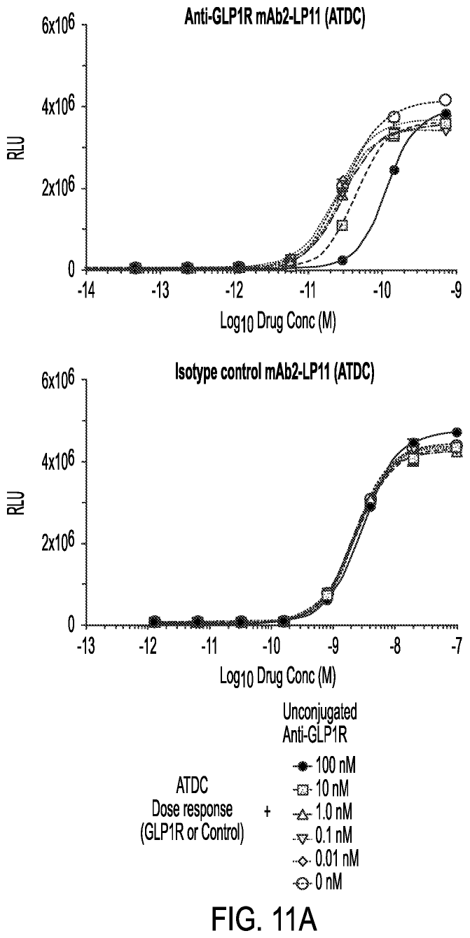

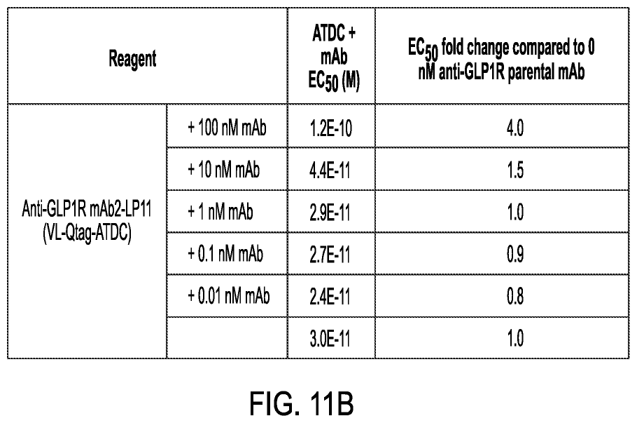

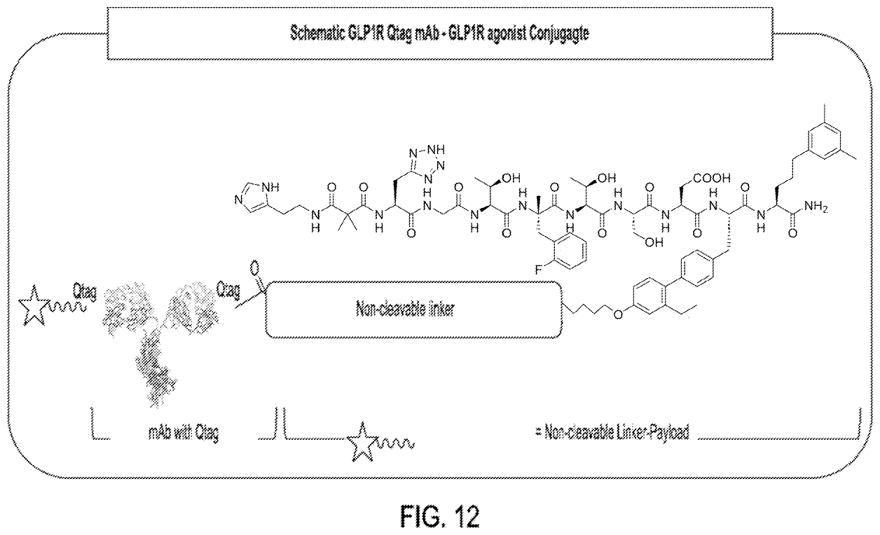

GLP1R agonist linker payloads were conjugated to anti-hGLP1R antibodies via N-terminal heavy or light chain Q tags. Several resulting anti-GLP1R antibody tethered drug conjugates (ATDCs) were tested for activity in the HEK293/CRE-Luc/hGLP1R reporter assay as described above for the free GLP1R payload and linker-payload agonists. As shown in Table 12, anti-GLP1R ATDCs increased ORE-dependent luciferase reporter activity in HEK293/CRE-Luc/hGLP1R cells with EC₅₀ values ranging from 21.7 μM to 112 μM and relative potency values (% P8) ranging from 14.5% to 126%. Most tested ATDCs reached similar max activity with relative S/N values (% P8) ranging from 87.4% to 158.4%. The anti-GLP1R ATDCs were inactive in reporter cells that did not express hGLP1R (HEK293/CRE-Luc). Non-binding ATDCs tended to be less active than the anti-GLP1R ATDCs with EC₅₀ values ranging from 1.92 nM to 74.6 nM and relative potency values (% P8) ranging from <0.1% to 1.4%. A selected set of results are presented in FIG. 10.

TABLE 12
CRE-Dependent Reporter Activity by Anti-GLP1R ATDCs in HEK293/CRE-Luc/hGLP1R Cells

| | | | HEK293/CRE-Luc/hGLP1R | | | | | | |
|---|---|---|---|---|---|---|---|---|---|
| | | | | Relative | | | | | |
| | | | | Potency | | Relative | | | |
| | mAb Q | | | (% P8 | | Signal:Noise | | HEK293/CRE-Luc | |
| Test Article | tag | LP | EC₅₀ | EC₅₀) | S/N | (% P8) | Experiment | EC₅₀ | S/N |
| Anti-GLP1R | VL N-term | LP11 | 2.73E−11 | 59.5 | 101.9 | 158.4 | A | >2.0E−08 | 1.2 |
| mAB2 | | | | | | | | | |
| Anti-GLP1R | VH N-term | LP11 | 1.12E−10 | 14.5 | 62.5 | 97.1 | A | >2.0E−08 | 0.9 |
| mAB6 | | | | | | | | | |
| GLP1 | None | None | 2.75E−11 | 58.9 | 88.7 | 137.9 | A | >2.0E−08 | 1.4 |
| P8 | None | None | 1.62E−11 | 100.0 | 64.3 | 100.0 | A | >2.0E−08 | 1.3 |
| Isotype | VH N-term | LP11 | 7.46E−08 | <0.1% | 69.9 | 108.7 | A | >2.0E−08 | 1.6 |
| Control mAb1 | | | | | | | | | |
| Isotype | VL N-term | LP11 | 1.24E−08 | 0.1 | 69.4 | 107.8 | A | >2.0E−08 | 1.2 |
| Control mAb2 | | | | | | | | | |
| COMP Anti- | VH N-term | LP4 | 6.29E−11 | 55.3 | 284.1 | 87.4 | D | >2.0E−8 | 1.6 |
| GLP1R mAb1 | | | | | | | | | |
| COMP Anti- | VH N-term | LP3 | 4.40E−11 | 62.1 | 271.7 | 115.2 | D | NT | NT |
| GLP1R mAb1 | | | | | | | | | |
| COMP Anti- | VH N-term | LP1 | 2.82E−11 | 96.7 | 213.0 | 90.3 | D | NT | NT |
| GLP1R mAb1 | | | | | | | | | |
| COMP Anti- | VH N-term | LP2 | 2.17E−11 | 126.0 | 227.3 | 96.4 | D | NT | NT |
| GLP1R mAb1 | | | | | | | | | |
| P8 | None | None | 2.73E−11 | 100.0 | 235.9 | 100.0 | D | NT | NT |
| Isotype | VH N-term | LP4 | 1.92E−09 | 1.4 | 252.0 | 106.8 | D | NT | NT |
| Control mAb3 | | | | | | | | | |
| Isotype | VH N-term | LP3 | 2.31E−09 | 1.2 | 218.8 | 92.8 | D | NT | NT |
| Control mAb3 | | | | | | | | | |
| Isotype | VH N-term | LP1 | 2.54E−09 | 1.1 | 235.0 | 99.6 | D | NT | NT |
| Control mAb3 | | | | | | | | | |
| Isotype | VH N-term | LP2 | 2.94E−09 | 0.9 | 285.0 | 120.8 | D | NT | NT |
| Control mAb3 | | | | | | | | | |

NT = not tested
> = EC₅₀ values could not be determined with accuracy because the binding did not reach saturation within the tested antibody concentration range. EC₅₀ is reported as greater than the highest tested concentration

In a separate experiment, the ability of unconjugated anti-GLP1R antibodies to compete for anti-GLP1R ATDC activity was assessed in the HEK293/CRE-Luc/hGLP1R reporter assay. In this experiment, reporter cells were incubated with a dose titration of the anti-GLP1R ATDC in the absence or presence of a constant amount (0.01, 0.1, 1.0, 10, or 100 nM) of the unconjugated anti-GLP1R antibody. The EC₅₀ values are reported in Table 13, and the fold-change in the EC₅₀ value relative to the ATDC alone condition (EC₅₀ fold-change) was calculated as follows: EC₅₀ of ATDC+unconjugated mAb/EC₅₀ of ATDC alone. As shown in Table 13, unconjugated mAb concentrations up to 10 nM had minimal impact on anti-GLP1R ATDC activity with the EC₅₀ fold-change values less than or equal to 1.5. The highest tested unconjugated antibody concentration of 100 nM reduced the EC₅₀ value by 4.0-fold. A non-binding control ATDC was not impacted by the presence of unconjugated anti-GLP1R antibody.

TABLE 13
CRE−Dependent Reporter Activity by Anti-GLP1R ATDCs in the
Presence of Unconjugated Anti-GLP1R Antibodies

| | Unconjugated mAb | | | |
|---|---|---|---|---|
| | constant | | ATDC | EC₅₀ |
| ATDC | concentration | | EC₅₀ (M) | fold-change |
| Anti-GLP1R | +100 | nM mAb | 1.2E−10 | 4.0 |
| mAb2 | +10 | nM mAb | 4.4E−11 | 1.5 |
| | +1 | nM mAb | 2.9E−11 | 1.0 |
| | +0.1 | nM mAb | 2.7E−11 | 0.9 |
| | +0.01 | nM mAb | 2.4E−11 | 0.8 |
| | 0 | nM mAb | 3.0E−11 | 1.0 |
| Isotype Control | +100 | nM mAb | 3.0E−09 | 1.3 |
| mAb2 | +10 | nM mAb | 2.4E−09 | 1.0 |
| | +1 | nM mAb | 2.2E−09 | 0.9 |
| | +0.1 | nM mAb | 2.50−09 | 1.1 |
| | +0.01 | nM mAb | 2.30E−09 | 1 |
| | 0 | nM mAb | 2.30E−09 | 1 |

### Example 12. Effects of Anti-GLP1R mAb ATDCs on Body Weight and Blood Glucose in Diet-Induced Obese GLP1R Humanized Mice

To determine body weight and blood glucose lowering effects of three anti-GLP1R antibody-tethered-drug conjugates (ATDCs) in obese animals, mice homozygous for the expression of human GLP1R in place of mouse GLP1R (referred to as GLP1R humanized mice) were placed on high-fat diet (60% kcal % fat) for 6 months. Forty-four, 9-month-old male, GLP1R humanized mice were stratified into six groups of five to eight mice, based on their day 0 body weights. After the stratification, each group was subcutaneously administered with 25 mg/kg of COMP anti-GLP1R mAb1-LP4 (n=8), anti-GLP1R mAb3-LP11 (n=8), anti-GLP1R mAb4-LP11 (n=5), control anti-GLP1R mAb (n=7), isotype control mAb ATDC (n=8) or reference compound (n=8) on day 0.

The control anti-GLP1R mAb used in this study is a high-affinity anti-GLP1R antibody, which does not activate or inactivate GLP1R, without any drug conjugation. The control anti-GLP1R mAb is antibody 5A10 described in US Publication No. US20060275288A1, which is incorporated herein by reference in its entirety. The control anti-GLP1R mAb does not have a Q-tag. COMP anti-GLP1R mAb1-LP4 comprises the same control anti-GLP1R mAb with a N-terminal heavy chain Q-tag. The isotype control mAb ATDC is a GLP1 peptide mimetic described herein, conjugated to an antibody that does not bind to any protein in GLP1R humanized mice. The reference compound has identical amino acid sequences to dulaglutide in GLP1 analogue and linker segments, but was made with a hFc with three mutations (P16E; V17A; G19 insert).

On days three and seven post administration and weekly from day fourteen to day fifty-six, body weights of the animals were recorded, and their blood glucose levels were measured with a handheld glucometer. Mean±SEM of percent changes in body weight from day 0 at each time point was calculated for each group and are shown in Table 14. Mean±SEM of blood glucose levels at each time point was calculated for each group and are shown in Table 15. Statistical analyses were performed by two-way ANOVA followed by Bonferroni post-hoc tests, comparing the control antibody group to the other five groups. The results are also presented in FIGS. 39 and 40.

Body weights and blood glucose levels were stable in animals administered with the control anti-GLP1R mAb, with nominal handling (i.e., bleeding, cage changes) related fluctuations. Compared to animals in the control anti-GLP1R mAb group, animals administered with the isotype control mAb ATDC showed no significant differences in percent body weight change or blood glucose levels. In animals administered with the reference compound, weight reductions were observed on days 3 and 7, whereas glucose was reduced only on day 3. In animals administered anti-GLP1R mAb ATDCs, weight reductions lasted for 42, 56, and 28 days, depending on which GLP1R ATDC was dosed, respectively, while glucose was lowered for 7 days for all GLP1R ATDCs tested.

In conclusion, single administration of the three anti-GLP1R mAb ATDCs tested leads to long-term weight loss in obese animals.

TABLE 14
Effects of GLP1R ATDCs on Percent Body Weight Changes in Obese GLP1R Humanized Mice

| | | | | | | | COMP anti- | | | | | |
|---|---|---|---|---|---|---|---|---|---|---|---|---|
| | Control anti- | | Isotype control | | Reference | | GLP1R mAb | | Anti-GLP1R mAb | | Anti-GLP1R mAb | |
| Time | GLP1R mAb | | mAb ATDC | | compound | | mAb1- LP4 | | mAb3-LP11 | | mAb4-LP11 | |
| (day) | Mean | SEM | Mean | SEM | Mean | SEM | Mean | SEM | Mean | SEM | Mean | SEM |
| 0 | 0 | 0 | 0 | 0 | 0 | 0 | 0 | 0 | 0 | 0 | 0 | 0 |
| 3 | −2.2 | 0.6 | −1.9 | 0.6 | −11.4** | 0.3 | −8.8 | 0.7 | −7.8 | 0.2 | −8.3 | 0.4 |
| 7 | −2.6 | 1.2 | −2.0 | 1.1 | −11.4** | 0.9 | −14.5**** | 0.6 | −11.3** | 0.9 | −11.3* | 0.8 |
| 14 | −3.3 | 1.5 | −2.2 | 1.8 | −6.6 | 1.4 | −19.6**** | 2.0 | −15.9**** | 1.7 | −13.2** | 1.7 |
| 20 | −2.2 | 1.1 | −1.8 | 2.0 | −4.8 | 1.4 | −18.0**** | 2.6 | −15.4**** | 2.0 | −10.9* | 3.0 |
| 28 | −0.7 | 1.3 | −1.5 | 2.1 | −2.3 | 1.1 | −13.7**** | 3.3 | −13.0**** | 2.3 | −8.9* | 4.1 |
| 35 | −0.3 | 1.5 | −1.4 | 2.3 | −0.9 | 1.4 | −11.2*** | 3.1 | −13.0**** | 3.0 | −7.2 | 4.0 |
| 42 | 0.1 | 1.4 | 0.7 | 2.3 | 0.7 | 1.5 | −8.3** | 2.0 | −12.6**** | 3.1 | −4.3 | 2.9 |
| 49 | −2.0 | 1.6 | 0.3 | 2.6 | 0.3 | 1.3 | −6.5 | 1.8 | −12.1*** | 2.8 | −3.1 | 2.0 |
| 56 | −1.7 | 1.1 | −0.7 | 2.1 | 0.1 | 1.6 | −5.0 | 1.5 | −13.2**** | 3.5 | −2.6 | 1.8 |

*P < 0.05,
**P < 0.01,
***P < 0.001,
****P < 0.0001, compared to the control anti-GLP1R mAb group.

TABLE 15
Effects of GLP1R ATDCs on Blood Glucose Levels in Obese GLP1R Humanized Mice

| | | | Isotype | | | | COMP anti- | | | | | |
|---|---|---|---|---|---|---|---|---|---|---|---|---|
| | Control anti- | | control mAb | | Reference | | GLP1R mAb | | Anti-GLP1R mAb | | Anti-GLP1R mAb | |
| Time | GLP1R mAb | | ATDC | | compound | | mAb1- LP4 | | mAb3-LP11 | | mAb4-LP11 | |
| (day) | Mean | SEM | Mean | SEM | Mean | SEM | Mean | SEM | Mean | SEM | Mean | SEM |
| 0 | 215 | 6 | 211 | 14 | 220 | 14 | 214 | 12 | 209 | 12 | 221 | 14 |
| 3 | 212 | 13 | 205 | 15 | 129**** | 10 | 144**** | 10 | 157** | 8 | 143*** | 8 |
| 7 | 202 | 12 | 198 | 13 | 163 | 6 | 158* | 10 | 154* | 9 | 155* | 16 |
| 14 | 186 | 5 | 196 | 9 | 206 | 12 | 167 | 8 | 162 | 7 | 178 | 9 |
| 20 | 200 | 10 | 191 | 10 | 198 | 8 | 176 | 7 | 184 | 7 | 205 | 4 |
| 28 | 207 | 11 | 213 | 16 | 204 | 13 | 197 | 9 | 190 | 8 | 188 | 8 |
| 35 | 219 | 12 | 220 | 12 | 207 | 11 | 211 | 10 | 200 | 10 | 222 | 17 |
| 42 | 208 | 11 | 217 | 17 | 229 | 12 | 208 | 7 | 196 | 12 | 224 | 10 |
| 49 | 226 | 15 | 207 | 12 | 228 | 13 | 215 | 10 | 214 | 12 | 226 | 13 |
| 56 | 217 | 14 | 198 | 12 | 205 | 12 | 196 | 10 | 204 | 4 | 229 | 16 |

*P < 0.05,
**P < 0.01,
***P < 0.001,
****P < 0.0001, compared to the control anti-GLP1R mAb group.

### REFERENCES

1. Zhang et al. Nature volume 546, pages 248-253(2017)
2. Chepurny et al. J Biol Chem. 2019 Mar. 8; 294(10):3514-3531.
3. De Graaf et al. Pharmacological Reviews October 2016, 68 (4) 954-1013.
4. Manandhar and Ahn. J. Med. Chem. 2015, 58, 3, 1020-1037
5. Jazayeri A, et al. Nature volume 546, pages 254-258 (2017)
6. Donnelly D, British Journal of Pharmacology, 2011: 166:27-41, PMID 21950636
7. GB2551945a
8. Jazayeri, A.; Rappas, M.; Brown, A. H.; Kean J.; Errey, J. C.; Robertson, N. J.; Fiez-Vandal, C.; Andrews, S. P.; Congreve, M.; Bortolato, A.; Mason, J. S.; Baig, A. H.; Teobald, I.; Dore, A. S.; Weir, M.; Cooke, R. M.; Marshall, F. H. Crystal structure of the GLP-1 receptor bound to a peptide agonist. Nature. 2017, 546, 254-258.
9. US2006/4222
10. Sureshbabu, V. V.; Venkataramanarao, R.; Naik, S. A.; G. Synthesis of tetrazole analogues of amino acids using Fmoc chemistry: isolation of amino free tetrazoles and their incorporation into peptides. Tetrahedron Letters 2007, 48, 7038-7041.
11. Ceretti, S.; Luppi, G.; Pol, S. D.; Formaggio, F.; Crisma, M.; Toniolo, C.; Tomasini, C. Total Synthesis of Sequential Retro-Peptide Oligomers. Eur. J. Org. Chem. 2004, 4188-4196.
12. WO2010/052253
13. US2003/114668
14. Colobert, F.; Mazery, R. D.; Solladié, G.; Carreño, M. C.; First Enantioselective Total Synthesis of (−)-Centrolobine. Org. Lett. 2002, 4, 1723-1725.
15. Dondoni, A.; Massi, A.; Aldhoun, M. Hantzsch-Type Three-Component Approach to a New Family of Carbon-Linked Glycosyl Amino Acids. Synthesis of C-Glycosylmethyl Pyridylalanines. J. Org. Chem. 2007, 72, 7677-7687.
16. Berezowska, I.; Chung, N. N.; Lemieux, C.; Wilkes, B. C.; Schiller, P. W. Agonist vs Antagonist Behavior of 5 Opioid Peptides Containing Novel Phenylalanine Analogues in Place of Tyr. J. Med. Chem. 2009, 52, 6941-6945.
17. US2015/380666
18. Campbell-Verduyn, L. S.; Mirfeizi, L.; Schoonen, A. K.; Rudi A. Dierckx, R. A.; Elsinga, P. H.; Feringa, B. L. Strain-Promoted Copper-Free “Click” Chemistry for 18F Radiolabeling of Bombesin. Angew. Chem. Int. Ed. 2011, 50, 11117-11120.
19. Crich, D.; Sana, K.; Guo, S. Amino Acid and Peptide Synthesis and Functionalization by the Reaction of Thioacids with 2,4-Dinitrobenzenesulfonamides. Org. Lett. 2007, 9, 4423-4426.
20. Wu, X. Y.; Stockdill, J. L.; Park, P. K.; Samuel J. Danishefsky, S. J. Expanding the Limits of Isonitrile-Mediated Amidations: On the Remarkable Stereosubtleties of Macrolactam Formation from Synthetic Seco-Cyclosporins. J. Am. Chem. Soc. 2012, 134, 2378-2384.
21. Du, J. J.; Gao, X. F.; Xin, L. M.; Lei, Z.; Liu, Z.; and Guo, J. Convergent Synthesis of N-Linked Glycopeptides via Aminolysis of w-Asp p-Nitrophenyl Thioesters in Solution. Org. Lett. 2016, 18, 4828-4831.
22. Naumovich, Y. A.; Golovanov, I. S.; Sukhorukov, A. Y.; Loffe, S. L. Addition of HO-Acids to N,N-Bis(oxy)enamines: Mechanism, Scope and Application to the Synthesis of Pharmaceuticals. Eur. J. Org. Chem. 2017, 41, 6209-6227.

### Example 13. The Effects of Anti-GLP1R mAb ATDCs on Body Weight and Blood Glucose in Diet-Induced Obese GLP1R Humanized Mice

To determine body weight and blood glucose lowering effects of four anti-GLP1R antibody-tethered-drug conjugates (ATDCs) of the invention (REGN7990-M3190; REGN8070-M3190; REGN7988-M3190 and REGN9268-M3190) in obese animals, mice homozygous for the expression of human GLP1R in place of mouse GLP1R (referred to as GLP1R humanized mice) were placed on high-fat diet (60% kcal % fat) for 5 months. Fifty, 6-month-old male, GLP1R humanized mice were stratified into seven groups of six to eight mice, based on their day 0 body weights. After the stratification, each group was subcutaneously administered with anti-GLP1R mAb REGN7990-M3190 (25 mg/kg or 165 nmol/kg; n=7), anti-GLP1R mAb REGN7990-M3190 (100 mg/kg or 660 nmol/kg; n=8), anti-GLP1R mAb REGN8070-M3190 (25 mg/kg or 167 nmol/kg; n=7), anti-GLP1R mAb REGN7988-M3190 (25 mg/kg or 165 nmol/kg; n=7), anti-GLP1R mAb REGN9268-M3190 (25 mg/kg or 165 nmol/kg; n=6), control anti-GLP1R mAb (25 mg/kg or 165 nmol/kg; n=8), or reference compound (25 mg/kg or 420 nmol/kg; n=7) on day 0. The animals that were administered with the reference compound on day 0 received subsequent doses of the same compound at the same dosage on days 3, 7, 10, 14, 17, 21 and 24, whereas the other six groups received one dosing only on day 0. A frequent, twice-a-week dosing schedule was applied to the reference compound due to its shorter duration of action compared to the other antibody-based compounds.

The control anti-GLP1R mAb ((REGN7989) will be referred as the Control thereafter) used in this study is a high-affinity anti-GLP1R antibody, which does not activate or inactivate GLP1R, with a glutamine-tag on N-terminal end of its heavy chain, but without any drug conjugation. The reference compound used in this study has identical amino acid sequences to dulaglutide in GLP1 analogue and linker segments, but was made with a hFc with three mutations (P16E; V17A; G19 insert).

On days three and seven post administration and weekly from day fourteen to day sixty-three, body weights of the animals were recorded, and their blood glucose levels were measured with a handheld glucometer. Mean±SEM of percent changes in body weight from day 0 at each time point was calculated for each group and are shown in Table 17. Mean±SEM of blood glucose levels at each time point was calculated for each group and are shown in Table 18. Statistical analyses were performed by two-way ANOVA followed by Bonferroni post-hoc tests, comparing the Control group to the other six groups.

Body weights and blood glucose levels were stable in animals administered with the Control, with nominal handling (i.e., bleeding, cage changes) related fluctuations. In animals administered with the reference compound, significant body weight reductions were observed between days 7 and 35, whereas blood glucose was significantly reduced on day 21. In animals administered with anti-GLP1R mAb REGN7990-M3190 at 25 mg/kg and 100 mg/kg, significant weight loss was observed between days 14 and 28 and days 7 and 42, respectively. Significant glucose reductions were observed with 100 mg/kg on days 3 and 14, but not with 25 mg/kg. Animals administered with anti-GLP1R mAb REGN8070-M3190 and REGN7988-M3190 at 25 mg/kg showed no significant changes in body weight or blood glucose compared to the Control group. In animals administered with anti-GLP1R mAb REGN9268-M3190 at 25 mg/kg, significant weight loss was observed between days 7 and 56, whereas blood glucose was significantly reduced on day 3.

In conclusion, single administration of the anti-GLP1R mAb ATDCs set forth below leads to long-term weight loss in obese animals.

TABLE 16
Antibodies used in this Example

| Antibody | Description |
|---|---|
| REGN7989 | Control anti-GLP1R mAb |
| REGN3355 | Reference compound; human GLP1-(G₄S)₃-hIgG4 |
| | variant peptide |
| REGN7990-M3190 | Anti-GLP1R mAb ATDC |
| REGN8070-M3190 | Anti-GLP1R mAb ATDC |
| REGN7988-M3190 | Anti-GLP1R mAb ATDC |
| REGN9268-M3190 | Anti-GLP1R mAb ATDC |

TABLE 17
Effects of GLP1R ATDCs on percent body weight changes in obese GLP1R
humanized mice

| | | | Reference compound | | | |
|---|---|---|---|---|---|---|
| | Control anti-GLP1R | | (25 mg/kg; | | | |
| | mAb | | 2×/wk for 4 wks)-human | | Anti-GLP1R mAb | |
| | (25 mg/kg; single) - | | GLP1-(G₄S)₃-hIgG4 | | REGN7990-M3190 | |
| Time | REGN7989 | | variant peptide | | (25 mg/kg; single) | |
| (day) | Mean | SEM | Mean | SEM | Mean | SEM |
| 0 | 0.0 | 0.0 | 0.0 | 0.0 | 0.0 | 0.0 |
| 3 | −2.6 | 1.2 | −11.1 | 0.8 | −7.9 | 0.5 |
| 7 | −3.1 | 1.1 | −14.1* | 1.5 | −12.1 | 0.8 |
| 14 | −3.2 | 1.4 | −18.7*** | 1.9 | −15.1** | 1.1 |
| 21 | −2.6 | 1.5 | −21.6**** | 2.3 | −15.4** | 1.7 |
| 28 | −1.3 | 0.5 | −23.3**** | 2.6 | −11.9* | 2.7 |
| 35 | 1.6 | 1.2 | −13.1*** | 2.7 | −7.1 | 2.3 |
| 42 | 0.4 | 1.1 | −7.2 | 3.0 | −4.3 | 1.9 |
| 49 | 2.9 | 1.3 | −2.4 | 3.4 | −0.5 | 2.0 |
| 56 | 4.2 | 1.8 | 1.7 | 3.9 | 1.8 | 2.3 |
| 64 | 3.3 | 1.8 | 3.4 | 3.8 | 2.9 | 2.1 |

| | Anti-GLP1R mAb | | Anti-GLP1R mAb | | Anti-GLP1R mAb | | | |
|---|---|---|---|---|---|---|---|---|
| Time | REGN7990-M3190 | | REGN8070-M3190 | | REGN7988-M3190 | | REGN9268-M3190 | |
| (day) | (100 mg/kg; single) | | (25 mg/kg; single) | | (25 mg/kg; single) | | (25 mg/kg; single) | |
| 0 | Mean | SEM | Mean | SEM | Mean | SEM | Mean | SEM |
| 3 | 0.0 | 0.0 | 0.0 | 0.0 | 0.0 | 0.0 | 0.0 | 0.0 |
| 7 | −9.5 | 0.7 | −8.6 | 1.3 | −6.4 | 1.0 | −8.6 | 0.7 |
| 14 | −14.1* | 0.8 | −9.9 | 2.3 | −6.6 | 1.2 | −14.8* | 1.2 |
| 21 | −19.0**** | 1.2 | −10.3 | 3.3 | −5.9 | 1.3 | −18.1*** | 2.2 |
| 28 | −21.0**** | 1.6 | −6.5 | 3.7 | −5.5 | 2.0 | −19.2**** | 3.4 |
| 35 | −18.3**** | 2.5 | −4.4 | 3.5 | −5.5 | 2.6 | −18.2**** | 3.9 |
| 42 | −15.0**** | 2.5 | 0.2 | 3.5 | −2.8 | 4.1 | −14.0*** | 4.2 |
| 49 | −10.7** | 2.2 | 2.5 | 3.1 | −1.0 | 5.7 | −11.6* | 3.9 |
| 56 | −6.3 | 2.5 | 4.5 | 3.1 | 0.9 | 6.8 | −7.3* | 4.4 |
| 64 | −3.5 | 2.4 | 6.1 | 2.9 | −0.2 | 6.9 | −6.2* | 3.3 |
| 0 | −1.3 | 2.2 | 5.1 | 2.6 | 0.5 | 6.4 | −5.3 | 3.5 |

*P < 0.05, **P < 0.01, ***P < 0.001, ****P < 0.0001, compared to the control anti-GLP1R mAb group.

TABLE 18
Effects of GLP1R ATDCs on blood glucose levels in obese GLP1R humanized mice

| | | | Reference | | | | | | | | | | | |
|---|---|---|---|---|---|---|---|---|---|---|---|---|---|---|
| | | | compound | | | | | | | | | | | |
| | | | (25 mg/kg; | | | | | | | | | | | |
| | | | 2×/wk for | | Anti-GLP1R | | Anti-GLP1R | | Anti-GLP1R | | Anti-GLP1R | | Anti-GLP1R | |
| | Control anti- | | 4 wks)- | | mAb | | mAb | | mAb | | mAb | | mAb | |
| | GLP1R mAb | | human GLP1- | | REGN7990- | | REGN7990- | | REGN8070- | | REGN7988- | | REGN9268- | |
| | (25 mg/kg; | | (G₄S)₃-hIgG4 | | M3190 | | M3190 | | M3190 | | M3190 | | M3190 | |
| | single)- | | variant | | (25 mg/kg; | | (100 mg/kg; | | (25 mg/kg; | | (25 mg/kg; | | (25 mg/kg; | |
| Time | REGN7989 | | peptide | | single) | | single) | | single) | | single) | | single) | |
| (day) | Mean | SEM | Mean | SEM | Mean | SEM | Mean | SEM | Mean | SEM | Mean | SEM | Mean | SEM |
| 0 | 203 | 12 | 207 | 17 | 208 | 11 | 211 | 9 | 207 | 15 | 205 | 11 | 193 | 11 |
| 3 | 194 | 9 | 154 | 14 | 154 | 7 | 148* | 12 | 200 | 12 | 188 | 18 | 133** | 12 |
| 7 | 212 | 17 | 174 | 11 | 176 | 4 | 179 | 7 | 188 | 12 | 213 | 19 | 175 | 14 |
| 14 | 220 | 9 | 180 | 5 | 177 | 8 | 176* | 7 | 212 | 5 | 222 | 12 | 190 | 12 |
| 21 | 227 | 9 | 181* | 15 | 198 | 14 | 190 | 7 | 212 | 8 | 234 | 12 | 204 | 5 |
| 28 | 208 | 11 | 176 | 7 | 203 | 12 | 187 | 10 | 219 | 14 | 207 | 15 | 194 | 12 |
| 35 | 196 | 8 | 223 | 1 | 203 | 8 | 175 | 9 | 204 | 17 | 200 | 15 | 228 | 5 |
| 42 | 185 | 10 | 220 | 16 | 208 | 12 | 177 | 8 | 206 | 10 | 211 | 23 | 204 | 16 |
| 49 | 205 | 10 | 230 | 12 | 206 | 15 | 170 | 8 | 198 | 12 | 219 | 14 | 177 | 7 |
| 56 | 214 | 13 | 217 | 12 | 219 | 14 | 197 | 7 | 203 | 12 | 220 | 15 | 200 | 19 |
| 64 | 220 | 8 | 230 | 15 | 211 | 12 | 196 | 7 | 215 | 8 | 214 | 13 | 221 | 18 |

*P < 0.05,
**P < 0.01, compared to the control anti-GLP1R mAb group.

### Example 14. The Effects of Anti-GLP1R mAb ATDC on Body Weight and Blood Glucose in Diet-Induced Obese GLP1R Humanized Mice

To determine body weight and blood glucose lowering effects of an anti-GLP1R antibody-tethered-drug conjugate (ATDC) of the invention in obese animals, mice homozygous for the expression of human GLP1R in place of mouse GLP1R (referred to as GLP1R humanized mice) were placed on high-fat diet (60% kcal % fat) for 5 months. Twenty-four, 7-month-old male, GLP1R humanized mice were stratified into three groups of eight mice, based on their day 0 body weights. After the stratification, each group was subcutaneously administered with either an anti-GLP1R mAb ATDC (REGN15869-M3190) at 25 mg/kg or 100 mg/kg or an isotype control antibody at 25 mg/kg on day 0.

On days three and seven post administration and weekly from the second week to the nineth week, body weights of the animals were recorded, and their blood glucose levels were measured with a handheld glucometer. Mean±SEM of percent changes in body weight from day 0 at each time point was calculated for each group and are shown in Table 20. Mean±SEM of blood glucose levels at each time point was calculated for each group and are shown in Table 21. Statistical analyses were performed by two-way ANOVA followed by Bonferroni post-hoc tests, comparing the isotype control antibody group to the other two groups.

Body weights and blood glucose levels were stable in animals administered with the isotype control antibody, with nominal handling (i.e., bleeding, cage changes) related fluctuations. In animals administered with anti-GLP1R mAb ATDC (REGN15869-M3190) at 25 mg/kg and 100 mg/kg, significant weight loss was observed between days 3 and 38 and days 7 and 38, respectively. Significant glucose reductions were observed on days 7,14, 21 and 42 with the 100 mg/kg dose of the anti-GLP1R mAb ATDC, and on day 14 with 25 mg/kg of the anti-GLP1R mAb ATDC.

In conclusion, single administration of the anti-GLP1R mAb ATDC of the invention (REGN 15869-M3190) leads to long-term weight and glucose lowering in obese animals.

TABLE 19
Antibodies used in this Example

| Antibody | Description in this report |
|---|---|
| REGN1945 | Isotype control antibody; Anti-FELD1 |
| REGN15869-M3190 | Anti-GLP1R mAb ATDC |

TABLE 20
Effects of GLP1R ATDC on percent body weight changes in obese GLP1R humanized mice

| | | | | | Anti-GLP1R mAb ATDC | |
|---|---|---|---|---|---|---|
| | Isotype control antibody- | | Anti-GLP1R mAb ATDC | | (100 mg/kg)- REGN15869- | |
| Time | REGN1945 | | (25 mg/kg)- REGN15869-M3190 | | M3190 | |
| (day) | Mean | SEM | Mean | SEM | Mean | SEM |
| 0 | 0 | 0 | 0 | 0 | 0 | 0 |
| 3 | −2.0 | 0.5 | −6.4 | 0.5 | −9.1* | 0.6 |
| 7 | −2.8 | 0.5 | −9.8* | 0.5 | −13.3** | 0.9 |
| 16 | −4.5 | 0.7 | −14.4** | 1.2 | −19.1**** | 1.5 |
| 24 | −2.8 | 1.6 | −14.1*** | 1.9 | −17.0**** | 2.2 |
| 31 | −1.7 | 1.9 | −12.2** | 2.6 | −13.8*** | 2.5 |
| 38 | −2.6 | 2.0 | −10.7* | 3.0 | −12.1** | 2.2 |
| 45 | −3.5 | 2.1 | −8.5 | 3.3 | −10.3 | 2.9 |
| 57 | 2.6 | 3.1 | −2.8 | 3.7 | −3.5 | 2.9 |
| 63 | 2.7 | 3.6 | −2.1 | 3.7 | −2.3 | 2.3 |

*P < 0.05,
**P < 0.01,
***P < 0.001,
****P < 0.0001, compared to the isotype control antibody group.

TABLE 21
Effects of GLP1R ATDC on blood glucose levels in obese GLP1R humanized mice

| | | | | | Anti-GLP1R mAb ATDC | |
|---|---|---|---|---|---|---|
| | Isotype control antibody- | | Anti-GLP1R mAb ATDC | | (100 mg/kg)- REGN15869- | |
| Time | REGN1945 | | (25 mg/kg)-REGN15869-M3190 | | M3190 | |
| (day) | Mean | SEM | Mean | SEM | Mean | SEM |
| 0 | 226 | 12 | 215 | 7 | 230 | 8 |
| 3 | 206 | 11 | 190 | 7 | 177 | 9 |
| 7 | 215 | 11 | 185 | 10 | 180* | 7 |
| 14 | 226 | 10 | 186* | 10 | 188* | 10 |
| 21 | 234 | 11 | 206 | 14 | 187** | 12 |
| 28 | 204 | 9 | 199 | 8 | 191 | 6 |
| 35 | 212 | 7 | 210 | 10 | 192 | 6 |
| 42 | 221 | 10 | 203 | 11 | 186* | 5 |
| 49 | 212 | 12 | 212 | 9 | 182 | 9 |
| 59 | 215 | 11 | 220 | 17 | 203 | 7 |
| 66 | 221 | 12 | 201 | 16 | 192 | 7 |

*P < 0.05,
**P < 0.01, compared to the isotype control antibody group.

### Example 15. Octet Cross-Competition Between GLP1R Monoclonal Antibodies for ATDCs

Binding competition between anti-GLP1R monoclonal antibodies (mAbs) was determined using a real time, label-free bio-layer interferometry (BLI) assay on the Octet RED384 biosensor platform (Pall ForteBio Corp.). The entire experiment was performed at 25° C. in 10 mM HEPES, 150 mM NaCl, 0.05% v/v Surfactant Tween-20, 1 mg/mL BSA, 0.02% NaN₃, pH7.4 (HBS-P) buffer with the plate shaking at a speed of 1000 rpm. To assess whether 2 mAbs are able to compete with one another for binding to their respective epitopes on GLP1R, the N-terminal ectodomain of human GLP1R expressed with a myc-myc hexahistidine tag (SEQ ID NO: 441) (referred to as hGLP1R-MMH) was first captured onto anti-penta-His antibody (HIS1K) coated Octet biosensor tips by submerging the biosensor tips for 30 seconds in wells containing 20 μg/mL solution of hGLP1R-MMH. The hGLP1R-MMH captured biosensor tips were then saturated with the first GLP1R mAb (subsequently referred to as mAb-1) by dipping into wells containing 50 μg/mL solution of mAb-1 for 5 minutes. The biosensor tips were then subsequently dipped into wells containing 50 μg/mL solution of second GLP1R mAb (subsequently referred to as mAb-2) for 3 minutes. The biosensor tips were washed in HBS-P buffer in between every step of the experiment. The real-time binding response was monitored during the entire course of the experiment and the binding response at the end of every step was recorded. The response of mAb-2 binding to hGLP1R-MMH pre-complexed with mAb-1 was compared and competitive/non-competitive behavior of different GLP1R mAbs was determined as shown in Table 22.

TABLE 22
Cross-competition between GLP1R mAbs

| | mAb-2 |
|---|---|
| | Competing |
| mAb-1 | with mAb-1 |
| REGN9267 | REGN9268 |
| | REGN7988 |
| | REGN5619 |
| | REGN9278 |
| | REGN7990 |
| REGN9268 | REGN9267 |
| | REGN7988 |
| | REGN5619 |
| | REGN9278 |
| | REGN7990 |
| REGN7988 | REGN9267 |
| | REGN9268 |
| | REGN5619 |
| | REGN9278 |
| | REGN7990 |
| REGN5619 | REGN9267 |
| | REGN9268 |
| | REGN7988 |
| | REGN9278 |
| | REGN7990 |
| REGN9278 | REGN9267 |
| | REGN9268 |
| | REGN7988 |
| | REGN5619 |
| | REGN7990 |
| REGN7990 | REGN9267 |
| | REGN9268 |
| | REGN7988 |
| | REGN5619 |
| | REGN9278 |
| REGN8070 | REGN8072 |
| REGN8072 | REGN8070 |

### Example 16. Biacore Binding Kinetics of GLP1R ATDCs

The equilibrium dissociation constant (K_{D}) for GLP1R binding to different unconjugated GLP1R monoclonal antibodies (mAbs) or to GLP1R antibody tethered drug conjugates (ATDCs) was determined using a real-time surface plasmon resonance biosensor using a Biacore 3000, Biacore S200, Biacore 4000, Biacore T-200, or a Sierra Sensor MASS-2 instrument. All binding studies were performed in 10 mM HEPES, 150 mM NaCl, 3 mM EDTA, and 0.05% v/v Surfactant Tween-20, pH 7.4 (HBS-ET) running buffer at 25° C. The Biacore CM5 sensor chip surface was first derivatized by amine coupling with human Fc specific mouse mAb (REGN2567) to capture different GLP1R mAbs or ATDCs. Different concentrations (100, 90, 33.3, 30, 11.1, 10, 3.7, 3.3 and 1.1 nM) of the N-terminal region of human GLP1R expressed with a myc-myc-hexahistidine tag (“hexahistidine tag” disclosed as SEQ ID NO: 441) (hGLP1R-MMH) prepared in HBS-ET running buffer were injected over the GLP1R mAb captured surface for 240 or 300 sec at a flow rate of 50 μL/min and their dissociation in HBS-ET running buffer was monitored for 10 minutes. At the end of each cycle, the GLP1R mAb or ATDC capture surface was regenerated using a 10 sec injection of 20 mM phosphoric acid.

The association rate (kₐ) and dissociation rate (k_{d}) were determined by fitting the real-time binding sensorgrams to a 1:1 binding model with mass transport limitation using Scrubber 2.0c curve-fitting software. Binding dissociation equilibrium constant (K_{D}) and dissociative half-life (t %₂) were calculated from the kinetic rates as:

$${K}_{D} ( M ) = \frac{k d}{k a} , and t 1 / 2 ( min ) = \frac{ln ( 2 )}{60 * kd}$$

Binding kinetics parameters for GLP1R binding to different GLP1R ATDCs of the invention at 25° C. are shown in Table 24 and for GLP1R binding to different unconjugated GLP1R mAbs of the invention at 25° C. are shown in Table 25.

As shown in Table 24, the GLP1R-ATDCs of the invention bound to hGLP1R-MMH at 25° C. with affinities ranging from 69 μM to 175 nM. As shown in Table 25, the GLP1R unconjugated Abs of the invention bound to hGLP1R-MMH at 25° C. with affinities ranging from 160 μM to 181 nM. Most ATDCs of the invention had a similar binding affinity to hGLP1R-MMH as their unconjugated parental Ab, however one ATDC (REGN7988-3190) had a weaker binding affinity as compared to its unconjugated parent Ab (H4H30439P), while two ATDCs (REGN8070-M3190 and REGN8072-M3190) had stronger binding affinities as compared to their unconjugated parent Abs (REGN8051 and REGN8052, respectively).

TABLE 23
ATDCs Correlated with Parental Ab

| ATDC | Ab used for ATDC | Parent |
|---|---|---|
| REGN7988-M3190 | H4H30439P w/ Qtag on | H4H30439P |
| | N-term of LC | |
| REGN7990-M3190 | H4H30452P w/ Qtag on | H4H30452P |
| | N-term of LC | |
| REGN8070-M3190 | REGN8051 w/ Qtag on | REGN8051 |
| | N-term of LC | |
| REGN8072-M3190 | REGN8052 w/ Qtag on | REGN8052 |
| | N-term of LC | |
| REGN9278-M3190 | H4H30341N w/ Qtag on | H2aM30341N |
| | N-term of LC | |
| REGN5619-M3190 | H4H30484P2 w/ Qtag on | H4H30484P2 |
| | N-term of LC | |
| REGN9267-M3190 | H4H30345N w/ Qtag on | H1M30345N |
| | N-term of HC | |
| REGN9268-M3190 | H4H30345N w/ Qtag on | H1M30345N |
| | N-term of LC | |

TABLE 24
Binding Kinetics Parameters of Different GLP1R
ATDCs Binding to hGLP1R-MMH at 25° C.

| | | 90 nM | | | | |
|---|---|---|---|---|---|---|
| | mAb | hGLP1R.mmh | | | | |
| | Capture | Bound | Kₐ | K_{d} | K_{D} | t_{1/2} |
| REGN # | (RU) | (RU) | (1/Ms) | (1/s) | (M) | (min) |
| REGN7988- | 158.2 ± 1.3 | 13.3 | 1.52E+05 | 4.88E−03 | 3.21E−08 | 2.4 |
| M3190 | | | | | | |
| REGN7990- | 101.9 ± 0.9 | 22.8 | 3.90E+05 | 7.85E−04 | 2.01 E−09 | 14.7 |
| M3190 | | | | | | |
| REGN8070- | 302.7 ± 6.6 | 86.7 | 8.78E+05 | 6.06E−05 | 6.90E−11 | 190.5 |
| M3190 | | | | | | |
| REGN8072- | 265.3 ± 7.1 | 77.3 | 1.74E+06 | 2.97E−04 | 1.70E−10 | 39.0 |
| M3190 | | | | | | |
| REGN9278- | 328.1 ± 1.1 | 55.7 | 1.16E+05 | 1.27E−04 | 1.10E−09 | 91.0 |
| M3190 | | | | | | |
| REGN5619- | 358.3 ± 8.3 | 35.3 | 1.54E+05 | 2.69E−02 | 1.75E−07 | 0.4 |
| M3190 | | | | | | |
| REGN9267- | 436.5 ± 5.1 | 110.2 | 3.53E+05 | 1.86E−04 | 5.28E−10 | 62.0 |
| M3190 | | | | | | |
| REGN9268- | 436.7 ± 14.9 | 114.2 | 3.19E+05 | 1.66E−04 | 5.22E−10 | 69.4 |
| M3190 | | | | | | |
| REGN15869- | 377.0 ± 1.1 | 99.7 | 5.49E+05 | 7.38E−04 | 1.34E−09 | 15.7 |
| M3190 | | | | | | |

TABLE 25
Binding Kinetics Parameters of Unconjugated GLP1R Monoclonal
Antibodies (Mabs) Binding to hGLP1R-MMH at 25° C.

| | | 90 nM | | | | |
|---|---|---|---|---|---|---|
| | mAb | hGLP1R.mmh | | | | |
| | Capture | Bound | Kₐ | K_{d} | K_{D} | t_{1/2} |
| REGN # | (RU) | (RU) | (1/Ms) | (1/s) | (M) | (min) |
| H4H30439P | 593.2 ± 3.7 | 152.4 | 2.77E+05 | 1.89E−04 | 6.81E−10 | 61.2 |
| H4H30452P | 331.1 ± 0.6 | 88.9 | 2.18E+05 | 1.18E−03 | 5.40E−09 | 9.8 |
| REGN8051 | 386 ± 0.8 | 73.7 | 6.19E+04 | 2.11E−04 | 3.41E−09 | 54.7 |
| REGN8052 | 365 ± 1.4 | 98.7 | 2.18E+05 | 1.20E−03 | 5.49E−09 | 9.6 |
| H2aM30341N | 286 ± 0.6 | 48.4 | 2.24E+05 | 7.74E−05 | 3.46E−10 | 149.3 |
| H4H30484P2 | 297 ± 2 | 26 | 2.15E+05 | 3.90E−02 | 1.81E−07 | 0.3 |
| H1M30345N | 332 ± 2.3 | 61.7 | 7.03E+05 | 1.13E−04 | 1.60E−10 | 102.5 |

### Example 17. Luciferase Reporter Assays

Glucagon-like peptide 1 receptor, GLP1R, is a member of the secretin family (Class B) of G protein-coupled receptors (GPCRs). Upon binding of its ligand, GLP1, GLP1R initiates a downstream signaling cascade through Gαs C-proteins that raises intracellular cyclic AMP (cAMP) levels, which leads to the transcriptional regulation of genes (Donnelly D, The structure and function of the glucagon-like peptide-1 receptor and its ligands, British Journal of Pharmacology, 2011: 166:27-41).

To test the activity of GLP1R agonist payloads, GLP1R agonist linker-payloads (LPs), and anti-GLP1R antibody tethered drug conjugates (ATDCs) of the invention, a cell-based cAMP responsive luciferase reporter assay was developed. To generate the assay cell line, the firefly luciferase gene was placed under the control of a cAMP response element (CRE) located upstream of a minimal promoter and transfected into HEK293 cells and referred to herein as HEK293/CRE-Luc cells. HEK293/CRE-Luc cells were then engineered to express full-length human GLP1R, cynomolgus GLP1R, human glucagon receptor (GCGR), human glucagon-like peptide 2 receptor (GLP2R), or human gastric inhibitory polypeptide receptor (GIPR) and are referred to herein as HEK293/CRE-Luc/hGLP1R, HEK293/CRE-Luc/mfGLP1R, HEK293/CRE-Luc/hGCGR, HEK293/CRE-Luc/hGLP2R, and HEK293/CRE-Luc/hGIPR, respectively.

For the assays, cells were seeded into 96 well plates at 10,000 or 20,000 cells/well in Optimem, 0.1% BSA, 100 units/ml Penicillin, 100 ug/ml Streptomycin, 292 ug/ml L-glutamine (assay media) and incubated overnight. Three-fold serial dilutions of free payloads or LPs were prepared in 100% DMSO, transferred to fresh assay media, and added to the cells at a final constant DMSO concentration of 0.2%. The last well in the plate served as a blank control containing only the assay media and 0.2% DMSO (untreated well) and was plotted as a continuation of the 3-fold serial dilution. Four to six hours later, luciferase activity was determined after the addition of One-Glo™ reagent (Promega, Cat #E6130) to each well. Relative light units (RLUs) were measured on an Envision luminometer (PerkinElmer) and EC₅₀ values were determined using a four-parameter logistic equation over a 12-point dose response curve (GraphPad Prism). The signal to noise (S/N) was determined by taking the ratio of the highest RLU on the dose response curve to the RLU in the untreated wells. EC₅₀ and S/N values are summarized in Table 26. Data was generated across several experiments (A-E) with M2361 serving as a reference standard in each experiment to calculate the payload relative potency ((M2361 EC₅₀/payload EC₅₀)*100)) and relative S/N ((payload SN/M2361 SN)*100)).

As shown in Table 26, payload and linker-payload EC₅₀ values ranged from 3.97 μM to 19.0 nM and relative potency (% M2361) ranged from 0.1% to 193.4% in HEK293/CRE-Luc/hGLP1R cells. Most tested payloads reached similar max activity with relative S/N values (% M2361) ranging from 68.6% to 137%. A few tested payloads (M2944, M2913, and M2383) had EC₅₀ values>20 nM. The GLP1 ligand was also included for reference, and GLP1 increased CRE-dependent luciferase activity in HEK293/CRE-Luc/hGLP1R cells with an EC₅₀ of 14.3 pM, relative potency of 37.2%, and relative S/N of 96.8%. All tested agonists had minimal impact on luciferase activity in the absence of human GLP1R expression in HEK293/CRE-Luc cells, with S/N values≤1.8 and EC₅₀ values>20.0 nM.

TABLE 26
CRE-Dependent Reporter Activity by GLP1R Payload and
Linker-Payload Agonistsin HEK293/CRE-Luc/Hglp1r Cells

| | HEK293/CRE-Luc/hGLP1R | | | | | | |
|---|---|---|---|---|---|---|---|
| | | Relative | | Relative | | | |
| | | Potency (% | | Signal:Noise | HEK293/CRE-Luc | | |
| Payload | EC₅₀ (M) | M2361 EC₅₀) | S/N | (% M2361) | EC₅₀ | S/N | Experiment |
| M2642 | 3.97E−12 | 133.8 | 75.2 | 114.3 | >2E−07 | 1.3 | A |
| M2800 | 5.15E−12 | 103.0 | 57.1 | 86.9 | >2E−07 | 1.6 | A |
| M2361 | 5.31E−12 | 100.0 | 65.8 | 100.0 | >2E−07 | 1.5 | A |
| M2361 | 8.01E−12 | 100.0 | 71.9 | 100.0 | >2E−08 | 1.3 | C |
| M3053 | 9.72E−12 | 54.6 | 55.3 | 84.1 | >2E−07 | 1.1 | A |
| M2739 | 1.0E−11 | 193.4 | 282.1 | 86.0 | NT | NT | B |
| M2761 | 1.14E−11 | 46.5 | 76.4 | 116.2 | >2E−07 | 1.2 | A |
| M2798 | 1.32E−11 | 40.3 | 67.8 | 103.1 | >2E−07 | 1.1 | A |
| M2743 | 1.40E−11 | 38.0 | 45.1 | 68.6 | >2E−07 | 1.2 | A |
| GLP1 | 1.43E−11 | 37.2 | 63.6 | 96.8 | >2E−07 | 1.3 | A |
| M3190 | 1.61E−11 | 33.1 | 74.5 | 113.3 | >2E−07 | 1.3 | A |
| M3151 | 1.74E−11 | 30.5 | 73.8 | 112.2 | >2E−07 | 1.3 | A |
| M2361 | 2.0E−11 | 100.0 | 327.9 | 100.0 | NT | NT | B |
| M2361 | 2.21E−11 | 100.0 | 209.9 | 100.0 | NT | NT | E |
| M2546 | 2.2E−11 | 87.7 | 279.2 | 85.1 | NT | NT | B |
| M3152 | 2.42E−11 | 21.9 | 54.8 | 83.4 | >2E−07 | 1.8 | A |
| M2747 | 2.51E−11 | 21.2 | 65.7 | 99.9 | >2E−07 | 1.4 | A |
| M2945 | 2.55E−11 | 31.4 | 71.8 | 99.9 | >2E−08 | 1.4 | C |
| M2361 | 2.73E−11 | 100.0 | 235.9 | 100.0 | NT | NT | D |
| M3241 | 3.16E−11 | 16.8 | 65.0 | 98.9 | >2E−07 | 1.0 | A |
| M3167 | 3.91E−11 | 13.6 | 51.7 | 78.7 | >2E−07 | 1.1 | A |
| M3120 | 4.67E−11 | 11.4 | 62.1 | 94.4 | >2E−07 | 1.1 | A |
| M2547 | 5.1E−11 | 38.6 | 267.8 | 81.7 | NT | NT | B |
| M3057 | 5.35E−11 | 9.9 | 78.5 | 119.4 | >2E−07 | 1.0 | A |
| M2877 | 5.42E−11 | 50.4 | 258.0 | 109.4 | NT | NT | D |
| M3056 | 6.13E−11 | 8.7 | 68.2 | 103.6 | >2E−07 | 1.4 | A |
| M2964 | 7.04E−11 | 7.5 | 48.1 | 73.2 | >2E−07 | 1.5 | A |
| M2663 | 7.42E−11 | 36.8 | 323.1 | 137.0 | NT | NT | D |
| M2799 | 8.37E−11 | 6.3 | 66.9 | 101.8 | >2E−07 | 1.6 | A |
| M2494 | 9.76E−11 | 28.0 | 293.2 | 124.3 | NT | NT | D |
| M2746 | 1.27E−10 | 4.2 | 50.6 | 76.9 | >2E−07 | 1.3 | A |
| M2399 | 1.27E−10 | 4.2 | 63.8 | 97.1 | >2E−07 | 1.7 | A |
| M2797 | 1.84E−10 | 2.9 | 74.9 | 113.9 | >2E−07 | 1.6 | A |
| M2985 | 2.17E−10 | 2.5 | 55.1 | 83.8 | >2E−07 | 1.1 | A |
| M2760 | 3.57E−10 | 1.5 | 69.5 | 105.6 | >2E−07 | 1.2 | A |
| M3055 | 3.66E−10 | 2.2 | 78.1 | 108.6 | >2E−08 | 1.3 | C |
| M2801 | 4.40E−10 | 1.2 | 48.9 | 74.4 | >2E−07 | 1.5 | A |
| M2758 | 6.02E−10 | 0.9 | 60.7 | 92.3 | >2E−07 | 1.0 | A |
| M2744 | 6.85E−10 | 0.8 | 54.8 | 83.4 | >2E−07 | 1.1 | A |
| M2912 | 7.38E−10 | 0.7 | 58.8 | 89.5 | >2E−07 | 1.3 | A |
| M3236 | 1.89E−09 | 0.3 | 48.4 | 73.6 | >2E−07 | 1.0 | A |
| M2745 | 1.95E−09 | 0.3 | 46.3 | 70.3 | >2E−07 | 1.2 | A |
| M2876 | 4.83E−09 | 0.2 | 79.3 | 110.2 | >2E−08 | 1.3 | C |
| M3240 | 7.83E−09 | 0.1 | 58.3 | 88.7 | >2E−07 | 1.0 | A |
| M2742 | 1.9E−08 | 0.1 | 306.2 | 93.4 | NT | NT | B |
| M2383 | >2E−07 | 0.0 | 293.3 | 89.4 | >2E−07 | 1.8 | B |
| M2913 | >2E−07 | 0.0 | 3.2 | 4.9 | >2E−07 | 1.1 | A |
| M2944 | >2E−08 | 0.0 | 8.2 | 11.4 | >2E−08 | 1.5 | C |

NT = not tested
> = EC₅₀ values could not be determined with accuracy because the binding did not reach saturation within the tested antibody concentration range. EC₅₀ is reported as greater than (>) the highest tested concentration

The anti-GLP1R ATDCs were tested for their activity in the HEK293/CRE-Luc/hGLP1R reporter assay as described above for the free GLP1R payload and linker-payload agonists. As shown in Table 27, anti-GLP1R ATDCs increased ORE-dependent luciferase reporter activity in HEK293/CRE-Luc/hGLP1R cells with EC₅₀ values ranging from 27.2 μM to 376 μM and relative potency values (% M2361) ranging from 4.3% to 59.5%. Most tested ATDCs reached similar max activity with relative S/N values (% M2361) ranging from 96.4.4% to 158.4%. The anti-GLP1R ATDCs were inactive in reporter cells that did not express human GLP1R (HEK293/CRE-Luc). Non-binding ATDCs tended to be less active than the anti-GLP1R ATDCs with EC₅₀ values ranging from 12.4 nM to 74.6 nM and relative potency values (% M2361) ranging from <0.1% to 0.1%.

TABLE 27
CRE-Dependent Reporter Activity by Anti-GLP1R ATDCs in HEK293/CRE-Luc/hGLP1R Cells

| | | | HEK293/CRE-Luc/hGLP1R | | | | | |
|---|---|---|---|---|---|---|---|---|
| | | | | Relative | | | | |
| | | | | Potency | | Relative | | |
| | | | | (% M2361 | | Signal:Noise | HEK293/CRE-Luc | |
| Test Article | mAb Q tag | LP | EC₅₀ (M) | EC₅₀) | S/N | (% M2361) | EC₅₀ (M) | S/N |
| REGN5619- | VL N-term | M3190 | 2.73E−11 | 59.5 | 101.9 | 158.4 | >2.0E−08 | 1.2 |
| M3190 | | | | | | | | |
| ATDC | | | | | | | | |
| REGN7988- | VL N-term | M3190 | 1.07E−10 | 15.1 | 90.1 | 140.0 | >2.0E−08 | 1.4 |
| M3190 | | | | | | | | |
| ATDC | | | | | | | | |
| REGN7990- | VL N-term | M3190 | 6.01E−11 | 27.0 | 86.9 | 135.1 | >2.0E−08 | 1.8 |
| M3190 | | | | | | | | |
| ATDC | | | | | | | | |
| REGN8070- | VL N-term | M3190 | 9.84E−11 | 16.5 | 71.8 | 111.7 | >2.0E−08 | 1.2 |
| M3190 | | | | | | | | |
| ATDC | | | | | | | | |
| REGN8072- | VL N-term | M3190 | 8.74E−11 | 18.6 | 80.7 | 125.4 | >2.0E−08 | 1.2 |
| M3190 | | | | | | | | |
| ATDC | | | | | | | | |
| REGN9267- | VH N-term | M3190 | 8.54E−11 | 19.0 | 68.9 | 107.0 | >2.0E−08 | 1.4 |
| M3190 | | | | | | | | |
| ATDC | | | | | | | | |
| REGN9268- | VL N-term | M3190 | 6.79E−11 | 23.9 | 66.8 | 103.8 | >2.0E−08 | 1.2 |
| M3190 | | | | | | | | |
| ATDC | | | | | | | | |
| REGN9278- | VL N-term | M3190 | 3.76E−10 | 4.3 | 73.8 | 114.8 | >2.0E−08 | 1.3 |
| M3190 | | | | | | | | |
| ATDC | | | | | | | | |
| GLP1 | None | None | 2.75E−11 | 58.9 | 88.7 | 137.9 | >2.0E−08 | 1.4 |
| M2361 | None | None | 1.62E−11 | 100.0 | 64.3 | 100.0 | >2.0E−08 | 1.3 |
| M3190 | None | M3190 | 3.69E−11 | 44.0 | 62.0 | 96.4 | >2.0E−08 | 1.3 |
| Isotype | VH N-term | M3190 | 7.46E−08 | <0.1% | 69.9 | 108.7 | >2.0E−08 | 1.6 |
| control ATDC | | | | | | | | |
| Isotype | VL N-term | M3190 | 1.24E−08 | 0.1 | 69.4 | 107.8 | >2.0E−08 | 1.2 |
| control ATDC | | | | | | | | |

NT = not tested
> = EC₅₀ values could not be determined with accuracy because the binding did not reach saturation within the tested antibody concentration range. EC50 is reported as greater than the highest tested concentration

In a separate experiment, several anti-GLP1R ATDCs were tested for activity in HEK293/CRE-Luc reporter cells expressing hGLP1R (HEK293/CRE-Luc/hGLP1R), cynomolgus GLP1R (HEK293/CRE-Luc/mfGLP1R), human GLP2R (HEK293/CRE-Luc/hGLP2R), human GCGR (HEK293/CRE-Luc/hGCGR), or human GIPR (HEK293/CRE-Luc/hGIPR). As shown in Table 28, anti-GLP1R ATDCs increased ORE-dependent luciferase reporter activity in HEK293/CRE-Luc/hGLP1R cells with EC₅₀ values ranging from 41.2 pM to 212 pM. All tested anti-GLP1R ATDCs similarly increased cynomolgus GLP1R activity with EC₅₀ values ranging from 37.3 pM to 250 pM. The anti-GLP1R ATDCs were inactive in reporter cells expressing human GCGR, human GLP2R, or human GIPR. Non-binding ATDCs tended to be less active than the anti-GLP1R ATDCs with EC₅₀ values of 21.8 nM and 71.2 nM. GLP1R, GLP2R, GCGR, and GIPR reporter activity was stimulated by their specific ligands with EC₅₀ values of 55.6 pM (GLP1), 1.09 nM (GLP2), 88.2 pM (GCG), and 362 pM (GIP), respectively, demonstrating that all expressed proteins are functionally active.

TABLE 28
CRE-Dependent Reporter Activity by Anti-GLP1R ATDCs in HEK293/CRE-
Luc/hGLP1R Cells, HEK293/CRE-Luc/mfGLP1R Cells, HEK293/CRE-Luc/hGLP2R
Cells, HEK293/CRE-Luc/hGCGR Cells, and HEK293/CRE-Luc/hGlPR Cells

| | | | HEK293/ | HEK293/ | HEK293/ | HEK293/ | HEK293/ | |
|---|---|---|---|---|---|---|---|---|
| | | | CRE-Luc/ | CRE-Luc/ | CRE-Luc/ | CRE-Luc/ | CRE-Luc/ | HEK293/ |
| | mAb Q | | hGLP1R | mfGLP1R | hGLP2R | hGCGR | hGlPR | CRE-Luc |
| Test Article | tag | LP | EC₅₀ (M) | EC₅₀ (M) | EC₅₀ (M) | EC₅₀ (M) | EC₅₀ (M) | EC₅₀ (M) |
| REGN5619- | VL N-term | M3190 | 4.12E−11 | 3.73E−11 | >2E−7 | >2E−7 | >2E−7 | >2E−7 |
| M3190 | | | | | | | | |
| ATDC | | | | | | | | |
| REGN7990- | VL N-term | M3190 | 7.24E−11 | 7.72E−11 | >2E−7 | >2E−7 | >2E−7 | >2E−7 |
| M3190 | | | | | | | | |
| ATDC | | | | | | | | |
| REGN8070- | VL N-term | M3190 | 9.40E−11 | 9.73E−11 | >2E−7 | >2E−7 | >2E−7 | >2E−7 |
| M3190 | | | | | | | | |
| ATDC | | | | | | | | |
| REGN8072- | VL N-term | M3190 | 1.14E−10 | 1.37E−10 | >2E−7 | >2E−7 | >2E−7 | >2E−7 |
| M3190 | | | | | | | | |
| ATDC | | | | | | | | |
| REGN9267- | VH N-term | M3190 | 2.12E−10 | 2.50E−10 | >2E−7 | >2E−7 | >2E−7 | >2E−7 |
| M3190 | | | | | | | | |
| ATDC | | | | | | | | |
| Isotype control | VH N-term | M3190 | 7.12E−08 | 8.77E−08 | >2E−7 | >2E−7 | >2E−7 | >2E−7 |
| ATDC | | | | | | | | |
| Isotype control | VL N-term | M3190 | 2.18E−08 | 2.68E−08 | >2E−7 | >2E−7 | >2E−7 | >2E−7 |
| ATDC | | | | | | | | |
| M3190 | None | M3190 | 2.58E−11 | 4.63E−11 | >2E−7 | >2E−7 | >2E−7 | >2E−7 |
| M2361 | None | None | 5.41E−12 | 1.04E−11 | >2E−7 | >2E−7 | >2E−7 | >2E−7 |
| GLP1 | None | None | 5.56E−11 | 6.47E−11 | >2E−7 | >2E−7 | >2E−7 | >2E−7 |
| GLP2 | None | None | >2E−7 | >2E−7 | 1.09E−09 | >2E−7 | >2E−7 | >2E−7 |
| GCG | None | None | 7.19E−09 | 1.05E−08 | >2E−7 | 8.82E−11 | >2E−7 | >2E−7 |
| GIP | None | None | >2E−7 | >2E−7 | >2E−7 | >2E−7 | 3.62E−10 | >2E−7 |

> = EC₅₀ values could not be determined with accuracy because the binding did not reach saturation within the tested antibody concentration range, and EC₅₀ is reported as greater than the highest tested concentration

In order to remove the glutamine at position 55 of the REGN7990 antibody light chain, a Q55E substitution was introduced to generate REGN15869. REGN15869 was conjugated to the M3190 linker payload and tested for activity in the HEK293/CRE-Luc/reporter assays as described previously. As shown in Table 29, the anti-GLP1R ATDCs, REGN15869-M3190 and REGN7990-M3190 increased ORE-dependent luciferase reporter activity in HEK293/CRE-Luc/hGLP1R cells with EC₅₀ values of 106 pM and 63.7 pM, respectively. The anti-GLP1R ATDCs REGN15869-M3190 and REGN7990-M3190 similarly increased cynomolgus GLP1R CRE-Luc reporter activity with EC₅₀ values of 202 pM and 257 pM. The anti-GLP1R ATDCs were inactive in reporter cells expressing human GCGR, human GLP2R, or human GIPR. Non-binding ATDCs tended to be less active than the anti-GLP1R ATDCs with an EC₅₀ value of 7.08 nM in human GLP1 expressing CRE-Luc reporter cells and an EC₅₀ value of 28.1 nM in cynomolgus GLP1R expressing CRE-Luc reporter cells. GLP1R, GLP2R, GCGR, and GIPR reporter activity was stimulated by their specific ligands with EC₅₀ values of 25.1 pM (GLP1), 1.09 nM (GLP2), 171 pM (GCG), and 172 pM (GIP), respectively, demonstrating that all expressed proteins are functionally active.

TABLE 29
CRE-Dependent Reporter Activity by the Anti-GLP1R ATDC REGN15869-M3190 in
HEK293/CRE-Luc/hGLP1R Cells, HEK293/CRE-Luc/mfGLP1R Cells, HEK293/CRE-Luc/hGLP2R
Cells, HEK293/CRE-Luc/hGCGR Cells, and HEK293/CRE-Luc/hGlPR Cells

| | | | HEK293/ | HEK293/ | HEK293/ | HEK293/ | HEK293/ | |
|---|---|---|---|---|---|---|---|---|
| | | | CRE-Luc/ | CRE-Luc/ | CRE-Luc/ | CRE-Luc/ | CRE-Luc/ | HEK293/ |
| | mAb Q | | hGLP1R | mfGLP1R | hGLP2R | hGCGR | hGlPR | CRE-Luc |
| Test Article | tag | LP | EC₅₀ (M) | EC₅₀ (M) | EC₅₀ (M) | EC₅₀ (M) | EC₅₀ (M) | EC₅₀ (M) |
| REGN15869- | VL N-term | M3190 | 1.06E−10 | 2.02E−10 | >3.0E−08 | >3.0E−08 | >3.0E−08 | >3.0E−08 |
| M3190 | | | | | | | | |
| ATDC | | | | | | | | |
| REGN15869 | VL N-term | None | >3.0E−08 | >3.0E−08 | >3.0E−08 | >3.0E−08 | >3.0E−08 | >3.0E−08 |
| unconjugated | | | | | | | | |
| Ab | | | | | | | | |
| REGN7990- | VL N-term | M3190 | 6.37E−11 | 2.57E−10 | >3.0E−08 | >3.0E−08 | >3.0E−08 | >3.0E−08 |
| M3190 | | | | | | | | |
| ATDC | | | | | | | | |
| REGN7990 | VL N-term | None | >3.0E−08 | >3.0E−08 | >3.0E−08 | >3.0E−08 | >3.0E−08 | >3.0E−08 |
| unconjugated | | | | | | | | |
| Ab | | | | | | | | |
| Isotype control | VL N-term | M3190 | 7.08E−09 | 2.81E−08 | >3.0E−07 | >3.0E−07 | >3.0E−07 | >3.0E−08 |
| ATDO | | | | | | | | |
| Isotype control | | | >3.0E−07 | >3.0E−07 | >3.0E−07 | >3.0E−07 | >3.0E−07 | >3.0E−08 |
| Ab | | | | | | | | |
| (unconjugated) | | | | | | | | |
| GLP1 | None | None | 2.51E−11 | 1.10E−10 | >3.0E−08 | >3.0E−08 | >3.0E−08 | >3.0E−08 |
| GCG | None | None | 1.00E−08 | ND | >3.0E−08 | 1.71E−10 | >3.0E−08 | >3.0E−08 |
| GLP2 | None | None | >3.0E−08 | ND | 1.09E−09 | >3.0E−08 | >3.0E−08 | >3.0E−08 |
| GIP | None | None | >3.0E−08 | ND | >3.0E−08 | >3.0E−08 | 1.72E−10 | >3.0E−08 |
| M3190 | None | M3190 | 6.01E−11 | 2.02E−10 | >3.0E−08 | >3.0E−08 | >3.0E−08 | >3.0E−08 |
| M2361 | None | None | 2.05E−11 | 6.59E−11 | >3.0E−08 | >3.0E−08 | >3.0E−08 | >3.0E−08 |

> = EC₅₀ values could not be determined with accuracy because the binding did not reach saturation within the tested antibody concentration range, and EC50 is reported as greater than the highest tested concentration

### Example 18. Flow Cytometry

GLP1R agonist linker payloads were conjugated to anti-GLP1R antibodies via N-terminal heavy or light chain Glutamine (Q) tags. Several resulting anti-GLP1R-GLP1R agonist antibody tethered drug conjugates (ATDCs) and their unconjugated parental antibodies were tested for cell-based binding activity. The cell surface binding of the anti-GLP1R ATDCs to human GLP1R (HEK293/CRE-Luc/hGLP1R), cynomolgus GLP1R (HEK293/CRE-Luc/mfGLP1R), and control cells lacking expression of GLP1R (HEK293/CRE-Luc) was assessed via flow cytometry. For the assay, 61,000-230,000 cells were suspended in PBS, w/2% FBS and 0.2% sodium azide (staining buffer) in 96 well V-bottom plates and incubated for 30 minutes at 4° C. with three-fold serial dilutions of the anti-GLP1R ATDCs, non-binding control ATDCs, or unconjugated anti-GLP1R antibodies. The last well in each row of the plate served as a blank control containing only secondary antibody and was plotted as a continuation of the 3-fold serial dilution. The cells were then washed once with staining buffer and were incubated with an APC conjugated Fab′₂ anti-human heavy+light IgG secondary antibody (Jackson Immunoresearch Cat #109-136-170) at 5 ug/mL for 30 minutes at 4° C. Cells were then washed once and stained with a cell viability dye (Live/dead Fixable Green Dead Cell Stain Kit for 488 nm excitation, Molecular Probes cat #L34970) at 1× in PBS for 20 minutes at 4° C. Cells were washed once in staining buffer, fixed for 20 minutes at 4° C. using a 50% solution of Cytofix (BD Biosciences, Cat #554655) diluted in PBS, and washed again in staining buffer. Samples were filtered through Pall 96 well plate PP/PE mesh filter system and collected in Costar 96 well plate. Samples were run on the iQue flow cytometer (Intellicyte) and results were analyzed using Forecyte analysis software (Intellicyte) to calculate the mean fluorescent intensity (MFI) after gating for live cells. EC₅₀ values were determined using a four-parameter logistic equation over a 10-point dose response curve (GraphPad Prism). The signal to noise (S/N) was determined by taking the ratio of the highest MFI on the dose response curve to the MFI in the secondary alone wells. The EC₅₀ values and S/N of each test article are shown in Table 30.

The tested anti-GLP1R antibodies and anti-GLP1R ATDCs bound HEK293/CRE-Luc/hGLP1R cells with EC₅₀ values ranging from 705 pM to 9.81 nM and S/N values from 114× to 560×. All tested anti-GLP1R antibodies and anti-GLP1R ATDCs antibodies bound HEK293/CRE-Luc/mfGLP1R cells with EC₅₀ values from 662 pM to 4.79 nM and S/N values from 103× to 697×. Non-binding control ATDCs bound weakly to HEK293/CRE-Luc/hGLP1R and HEK293/CRE-Luc/mfGLP1R cells with EC₅₀ values≥100 nM and S/N values≤15×. All anti-GLP1R antibodies and anti-GLP1R ATDCs bound weakly to parental HEK293/CRE-Luc cells with EC₅₀ values>100 nM and S/N values≤26×.

TABLE 30
Anti-GLP1R Antibody and Anti-GLP1R ATDC Binding to HEK293/CRE-Luc/GLP1R Cells.

| | | | | HEK293/CRE-Luc/ | | HEK293/CRE-Luc/ | | | | |
|---|---|---|---|---|---|---|---|---|---|---|
| | | | | hGLP1R | | mfGLP1R | | HEK293/CRE-Luc | | |
| Parent | | | | Flow | Flow | Flow | Flow | Flow | Flow | |
| mAb ID | Mab | Q tag | LP | EC50 | S/N | EC50 | S/N | EC50 | S/N | Experiment |
| 30484P2 | H4H30484P2 | None | None | 1.98E−09 | 208.4 | 9.63E−10 | 283.6 | >1E−07 | 12.8 | C |
| | REGN5619 | VL N-term | None | 5.68E−09 | 239.7 | 2.19E−09 | 155.2 | >1E−07 | 1.6 | A |
| | unconjugated Ab | | | | | | | | | |
| | REGN5619 | VL N-term | M3190 | 3.18E−09 | 172.6 | 2.28E−09 | 128.4 | >1E−07 | 8.0 | A |
| | ATDC | | | | | | | | | |
| 30439P | H4H30439P | None | None | 7.69E−10 | 140.5 | NT | NT | NT | NT | D |
| | REGN7988 | VL N-term | None | 1.89E−09 | 397.7 | 6.62E−10 | 467.2 | >1E−07 | 9.2 | C |
| | unconjugated Ab | | | | | | | | | |
| | REGN7988 | VL N-term | M3190 | NT | NT | NT | NT | NT | NT | NT |
| | ATDC | | | | | | | | | |
| 30452P | H4H30452P | None | None | 1.68E−09 | 114.6 | NT | NT | NT | NT | D |
| | REGN7990 | VL N-term | None | 1.89E−09 | 256.3 | 9.88E−10 | 204.8 | >1E−07 | 1.2 | A |
| | unconjugated Ab | | | | | | | | | |
| | REGN7990 | VL N-term | M3190 | 9.81E−09 | 116.9 | 4.79E−09 | 103.0 | >1E−07 | 1.6 | A |
| | ATDC | | | | | | | | | |
| 8051 | REGN8051 | None | None | 7.39E−09 | 560.2 | 4.12E−09 | 493.7 | >1E−07 | 18.0 | C |
| | REGN8070 | VL N-term | None | 3.44E−09 | 322.0 | 2.05E−09 | 246.5 | >1E−07 | 1.4 | A |
| | unconjugated Ab | | | | | | | | | |
| | REGN8070 | VL N-term | M3190 | 4.39E−09 | 166.4 | 3.71E−09 | 155.3 | >1E−07 | 2.0 | A |
| | ATDC | | | | | | | | | |
| 8052 | REGN8052 | None | None | 7.05E−10 | 294.0 | 2.13E−09 | 504.7 | >1E−07 | 25.8 | C |
| | REGN8072 | VL N-term | None | 1.42E−09 | 275.2 | 1.09E−09 | 213.0 | >1E−07 | 2.1 | A |
| | unconjugated Ab | | | | | | | | | |
| | REGN8072 | VL N-term | M3190 | 3.88E−09 | 173.3 | 3.39E−09 | 147.8 | >1E−07 | 2.2 | A |
| | ATDC | | | | | | | | | |
| 30345N | H1M30345N | None | None | 1.64E−09 | 510.1 | 3.54E−09 | 697.0 | >1E−07 | 12.2 | C |
| | REGN9267 | VH N-term | None | 9.44E−10 | 390.5 | 6.68E−10 | 324.4 | >1E−07 | 1.9 | A |
| | unconjugated Ab | | | | | | | | | |
| | REGN9268 | VL N-term | None | 1.09E−09 | 376.8 | 8.10E−10 | 302.3 | >1E−07 | 1.5 | A |
| | unconjugated Ab | | | | | | | | | |
| | REGN9267 | VH N-term | M3190 | 5.08E−09 | 269.4 | 4.44E−09 | 212.2 | >1E−07 | 5.5 | A |
| | ATDC | | | | | | | | | |
| | REGN9268 | VL N-term | M3190 | 2.99E−09 | 229.5 | 2.80E−09 | 186.9 | >1E−07 | 4.2 | A |
| | ATDC | | | | | | | | | |
| 30341N | H2aM30341N | None | None | 2.75E−09 | 595.4 | 2.10E−09 | 645.6 | >1E−07 | 11.8 | C |
| | REGN9278 | VL N-term | None | 4.21E−09 | 435.5 | 2.49E−09 | 547.6 | >1E−07 | 11.9 | C |
| | unconjugated Ab | | | | | | | | | |
| | REGN9278 | VL N-term | M3190 | NT | NT | NT | NT | NT | NT | NT |
| | ATDC | | | | | | | | | |
| Isotype | Isotype control | None | None | NT | NT | NT | NT | NT | NT | NT |
| control | Ab | | | | | | | | | |
| (Control) | Isotype control | VH N-term | None | >1E−07 | 2.4 | >1E−07 | 1.7 | >1E−07 | 1.6 | A |
| | Ab | | | | | | | | | |
| | unconjugated Ab | | | | | | | | | |
| | Isotype control | VH N-term | M3190 | >1E−07 | 2.0 | >1E−07 | 2.4 | >1E−07 | 1.3 | A |
| | Ab | | | | | | | | | |
| | ATDC | | | | | | | | | |
| | Isotype control | VL N-term | None | >1E−07 | 1.2 | >1E−07 | 1.8 | >1E−07 | 1.2 | A |
| | Ab | | | | | | | | | |
| | unconjugated Ab | | | | | | | | | |
| | Isotype control | VL N-term | M3190 | >1E−07 | 14.3 | >1E−07 | 11.2 | >1E−07 | 1.8 | A |
| | Ab | | | | | | | | | |
| | ATDC | | | | | | | | | |

NT = not tested
> = EC₅₀ values could not be determined with accuracy because the binding did not reach saturation within the tested antibody concentration range. EC₅₀ is reported as greater than the highest tested concentration

### Example 19. Analysis of GLP1R Unconjugated Ab and their Respective ATL Samples by Reduced Peptide Mapping

GLP1R unconjugated Ab and their respective ATL samples were analyzed by reduced peptide mapping to identify crosslinking species which contribute to HMW size variants. For this assay samples were denatured, reduced, and alkylated with Tris-(2-carboxyethyl) phosphine hydrochloride (TCEP) and iodoacetamide (IAA), respectively. The reduced samples were digested with trypsin for 4 hours at 37° C. followed by quenching with trifluoroacetic acid (TFA). Tryptic digests (5 μg samples) were loaded onto a C18 column.

Peptides eluted from the C18 column were analyzed by UV absorption at 214 nm and subjected to MS acquisition using a Q Exactive™ Plus Hybrid Quadrupole-Orbitrap™ Mass Spectrometer (Thermo). The source parameters were set as follows: spray voltage, 3.8 kV; auxiliary gas, 10; auxiliary gas temperature, 250° C.; capillary temperature, 320° C.; and S-lens RF level, 50. Data-dependent acquisition (DDA) was performed with one full MS scan from 300 m/z to 2000 m/z followed by five sequential MS/MS scans. Full MS scans were collected at a resolution of 70,000 with AGC target of 1 E6. The MS/MS scans were collected at a resolution of 17,500 with an AGC target of 1 E5. The isolation width was 4 m/z and the normalized collision energy (NCE) was set at 27.

Peptides were identified by database searches against mAb sequences using Byonic (version 4.6.1, Protein Metrics, San Carlos, CA). Common mAb post-translational modifications (PTMs) were included in the search parameters as variable modifications; carbamidomethylation of cysteine was included as a fixed modification. Q-K crosslinking associated with a loss of NH₃ (−17.0265 Da), glutamine deamidation (+0.9840 Da) and conjugation of LP were included as variable modifications as well. Peptide quantification was performed using Skyline software (version 21.2, MacCoss Lab Software, Seattle, WA, USA).

As shown in Table 31, multiple forms of the heavy chain (HC)C-terminal peptide were identified and quantified. As expected, Intact C-terminal lysine (K) was only observed in REGN15869 and its corresponding ATL, REGN15869-M3190, but not observed in REGN18121 or REGN18123 or their corresponding ATLs. The crosslinked peptide between HC C-terminal K and target glutamine (Q) was only observed in REGN15869-M3190, but not in REGN18121-M3190 or REGN18123-M3190, which explains the difference in the level of HMW between the three ATLs.

TABLE 31
Relative abundances of PTMs identified at HC C-terminus. Quantification is based on the peak area of extracted ion chromatography
of each peptide. Bolded residues are the residues with post translational modifications (“ND” means not detected).

| | | | | | REGN18121- | | REGN18123- |
|---|---|---|---|---|---|---|---|
| | | REGN1586 9 | REGN15869- M3190 | REGN18121 | M3190 | REGN18123 | M3190 |
| Sequence | PTM name | Modification % | Modification % | Modification % | Modification % | Modification % | Modification % |
| SLSLSLGK | C-term Lys | 89.1% | 92.4% | 99.1% | 99.1% | 0.0% | 0.0% |
| (SEQ ID NO: | loss | | | | | | |
| 418) | | | | | | | |
| SLSLSLGK | C-term with | 10.1% | 6.7% | 0.0% | 0.0% | ND | ND |
| (SEQ ID NO: | intact Lys | | | | | | |
| 418) | | | | | | | |
| SLSLSLGK | C-term Gly- | 0.0% | 0.0% | 0.1% | 0.1% | 100.0% | 100.0% |
| (SEQ ID NO: | Lys loss | | | | | | |
| 418) | | | | | | | |
| SLSLSLGK | C-term Gly | 0.8% | 0.8% | 0.9% | 0.8% | ND | ND |
| (SEQ ID NO: | loss + amide | | | | | | |
| 418) | | | | | | | |
| SLSLSLGK | Crosslinked to | ND | 0.1% | ND | ND | ND | ND |
| (SEQ ID NO: | target Q | | | | | | |
| 418) | | | | | | | |

### Example 20. Measurements of High Molecular Weight (HMW) Species in GLP1R Antibody Tethered Ligand (ATL) Samples

High molecular weight (HMW) species in GLP1R antibody tethered ligand (ATL) samples were measured using Size Exclusion-Ultra Performance Liquid Chromatography (SE-UPLC). For this procedure, multiple lots of each ATL were examined. The method used a ACQUITY UPLC BEH200 SEC column (1.7 μm, 4.6×300 mm, Waters cat. #186005226) and UV or PDA detector. Mobile phase used was 10 mM Sodium Phosphate, 1.0 M Sodium Perchlorate, pH 6.0, 5% (v/v) isopropanol with operation in isocratic mode at 0.3 mL/min and ambient temperature. Samples were injected undiluted at a target column loading of 40 μg. Data acquisition was performed at a wavelength of 280 nm and relative peak distribution was calculated using peak area.

GLP1R ATLs prepared with the antibodies REGN18121 and REGN18123 consistently showed lower HMW species (A2-3%) than those with prepared with REGN15869. This suggests that removal of C-terminal lysine from the antibody decreases antibody cross-linking propensity mediated by transglutaminase as per expectations.

TABLE 32
Percent High Molecular Weight Species in GLP1R Antibody Tethered
Ligand Samples as Measured by SE-UPLC

| ATL Lot# | HMW by SE-UPLC (%) |
|---|---|
| REGN15869-M3190 lot 1 | 6.4 |
| REGN15869-M3190 lot 2 | 7.5 |
| REGN15869-M3190 lot 3 | 7.1 |
| REGN18121-M3190 lot 1 | 5.4 |
| REGN18121-M3190 lot 2 | 5.1 |
| REGN18121-M3190 lot 3 | 4.1 |
| REGN18121-M3190 lot 4 | 5.6 |
| REGN18121-M3190 lot 5 | 5.5 |
| REGN18121-M3190 lot 6 | 4.9 |
| REGN18121-M3190 lot 7 | 5.0 |
| REGN18121-M3190 lot 8 | 5.0 |
| REGN18121-M3190 lot 9 | 5.2 |
| REGN18123-M3190 lot 1 | 5.2 |
| REGN18123-M3190 lot 2 | 5.1 |
| REGN18123-M3190 lot 3 | 4.3 |
| REGN18123-M3190 lot 4 | 4.7 |

### Example 21. Measurement of Purity for Covalent High Molecular Weight (HMW) Species in GLP1R Antibody Tethered Ligand (ATL) Samples Using Non-Reduced Microchip Capillary Electrophoresis (NR-MCE)

Purity, covalent high molecular weight (HMW) species in GLP1R antibody tethered ligand (ATL) samples were measured using Non-Reduced Microchip Capillary Electrophoresis (NR-MCE).

For this procedure, multiple lots of each ATL were examined. Protein samples were diluted to 0.16 g/L in molecular biology grade water and mixed with a non-reducing solution for a final concentration of 8 mM N-ethylmaleimide (NEM, Sigma, Cat. No. 04259), 12 mM sodium phosphate (J. T. Baker Cat. No. 3802-05 and VWR Cat. No. VWRB0348) and 0.24% LDS (lithium dodecyl sulfate, Sigma Cat. No. L9781). Denaturation occurred at 60° C. for 10 minutes followed by a labeling reaction, performed by the addition of an 8 μM dye followed by an incubation step at 35° C. for 15 minutes. The reaction was quenched by the addition of the stop solution. PICO Protein Reagent Kit (Perkin Elmer Cat. No. 760498), Protein Express LabChip (Perkin Elmer Cat. No. 760499) and Lab Chip GXII Touch instrument were used for sample preparation and analysis.

GLP1R ATLs prepared with REGN18121 and REGN18123 consistently showed lower covalent HMW species (A2-3%) than those prepared with REGN15869. This suggests that removal of C-terminal lysine from the parental antibody decreases antibody cross-linking propensity mediated by transglutaminase as per expectations.

TABLE 33
Percent Covalent High Molecular Weight Species in GLP1R Antibody
Tethered Ligand Samples as Measured by NR-MCE

| ATL Lot# | HMW by NR-MCE (%) |
|---|---|
| REGN15869-M3190 lot 1 | 3.5 |
| REGN15869-M3190 lot 2 | 4.1 |
| REGN18121-M3190 lot 1 | 1.8 |
| REGN18121-M3190 lot 2 | 2.4 |
| REGN18121-M3190 lot 3 | 2.4 |
| REGN18121-M3190 lot 4 | 2.3 |
| REGN18121-M3190 lot 5 | 1.9 |
| REGN18123-M3190 lot 1 | 1.3 |

### Example 22. Effects of Anti-GLP1R Antibody-Tethered-Ligands (ATLs) on Body Weight and Plasma Glucose in Non-Human Primates

To determine effects of anti-GLP1R antibody-tethered-ligands (ATLs) of the invention on body weight and plasma glucose in non-human primates, male, obese and diabetic cynomolgus monkeys (Macaca fascicularis) were administered with weekly ascending doses of ATLs.

Following training and acclimation in a period of seven weeks, ten (10) animals were selected all with baseline mean±SEM body weight of 10.4±0.6 kg, fasting plasma glucose of 196±20 mg/dL, and weekly total energy intake of 5451±321 kcal. Non-human primates were stratified into two groups of five, based on the baseline body weight, fasting glucose, and energy intake. Each group of animals was subcutaneously administered with either REGN7990-M3190 or REGN9268-M3190, respectively, at weekly ascending doses of 0.1, 0.5, 2.0, 6.0 and 12.0 mg/kg.

For three weeks prior to the initial compound administration and one week post the last administration, body weight, fasting glucose, and total energy intake of each animal were recorded weekly. Plasma glucose levels were measured using Roche C311 and C501 biochemical analyzer. Mean±SEM of percent changes in body weight from baseline at each time point was calculated for each group and are shown in Table 34. Baseline body weight of each animal was defined as the body weight recorded two days prior to the first compound administration. Mean±SEM of percent changes in fasting plasma glucose levels from baseline at each time point was calculated for each group and are shown in Table 35. Baseline plasma glucose level of each animal was defined as the mean of three weekly plasma glucose measurements prior to the first compound administration. Mean±SEM of percent changes in weekly energy intake from baseline at each time point was calculated for each group and are shown in Table 36. Baseline energy intake of each animal was defined as the mean of two weekly energy intake prior to the first compound administration. Statistical analyses were performed by two-way ANOVA followed by Dunnett post-hoc tests, comparing the mean of each data point to the baseline value for each group.

In obese and diabetic monkeys administered with REGN7990-M3190, significant reductions in body weight, plasma glucose and energy intake were observed at all timepoints after 2 mg/kg dose. In monkeys administered with REGN9268-M3190, significant reductions in body weight were observed at the two timepoints after 6 mg/kg administration and accompanied lowering of energy intake after 0.5 mg/kg dose. Plasma glucose reductions in these animals did not reach statistical significance.

In conclusion, the data demonstrate that GLP1R ATLs in this invention reduce body weight, plasma glucose and/or food intake in male, obese and diabetic non-human primates.

TABLE 34
Effects of GLP1R ATLs on percent changes in body weight in male,
obese and diabetic non-human primates

| Time | REGN7990-M3190 | | REGN9268-M3190 | |
|---|---|---|---|---|
| (weeks) | Mean | SEM | Mean | SEM |
| 0 | 0 | 0 | 0 | 0 |
| 1 | −1.2 | 1.2 | −0.7 | 0.7 |
| 2 | −2.6 | 1.9 | −1.8 | 0.9 |
| 3 | −6.1** | 2.2 | −4.4 | 1.4 |
| 4.1 | −9.3*** | 2.3 | −6.7** | 1.6 |
| 5.3 | −11.1**** | 2.6 | −9.1*** | 1.9 |

**P < 0.01, ***P < 0.001, ****P < 0.0001, compared to the baseline.

TABLE 35
Effects of GLP1R ATLs on percent changes in fasting glucose in male,
obese and diabetic non-human primates

| Time | REGN7990-M3190 | | REGN9268-M3190 | |
|---|---|---|---|---|
| (weeks) | Mean | SEM | Mean | SEM |
| 0 | 0 | 0 | 0 | 0 |
| 1 | −1.6 | 4.0 | −0.8 | 7.2 |
| 2 | −21.4 | 8.3 | −13.2 | 4.8 |
| 3 | −35.0** | 5.0 | −26.0 | 10.9 |
| 4 | −40.6** | 5.2 | −24.8 | 12.4 |
| 5 | −31.6* | 7.1 | −25.6 | 12.4 |

*P < 0.05, **P < 0.01, compared to the baseline.

TABLE 36
Effects of GLP1R ATLs on percent changes in weekly energy intake in
male, obese and diabetic non-human primates

| Time | REGN7990-M3190 | | REGN9268-M3190 | |
|---|---|---|---|---|
| (weeks) | Mean | SEM | Mean | SEM |
| 0 | 0 | 0 | 0 | 0 |
| 1 | −20.5 | 9.1 | −22.5 | 6.7 |
| 2 | −33.7 | 10.1 | −36.8* | 6.7 |
| 3 | −54.9* | 9.8 | −48.6* | 8.8 |
| 4 | −66.9** | 9.0 | −63.1** | 6.7 |
| 5 | −57.0* | 12.9 | −63.3*** | 4.9 |

*P < 0.05, **P < 0.01, ***P < 0.001, compared to the baseline.

### Example 23. Determination of the Structure of GLP-1R Antibody Tethered Ligands (ATLs) or their Parent Abs in the Presence of Linker Payload (“Untethered”) for Binding to GLP-1R Complexed with G Proteins

In To determine the structure of GLP-1R antibody tethered ligands (ATLs) or their parent Abs in the presence of linker payload (“untethered”) for binding to GLP-1R complexed with G proteins, cryogenic electron microscopy (cryoEM) experiments were performed.

### GLP-1R Complex Production

Expression constructs adapted from literature were synthesized and cloned by GenScript into pFastBac1 Expression Vectors (GLP-1R and GNAS) or into the same pFastBac Dual Expression Vector (GBB1 and GNG2). Subsequently, these plasmids were used to insert the genes into bacmids in MAX Efficiency™ DH10Bac Competent Cells (Thermo Fisher, 10361012), which were in turn used to make baculovirus in Sf9 cells (Gibco, 11496015) cultured in SF900 III SFM media (Thermo Fisher 12658027). Descriptions of the genes and modifications are as follows:

GLP1R (encoding GLP-1R; reference UniProt: P43220) - The GLP1R
construct used comprises the following sequence: N-terminal HA signal
peptide (MKTIIALSYIFCLVFA (SEQ ID NO: 442))-FLAG tag
(DYKDDDD (SEQ ID NO: 443))-3C protease recognition site
(LEVLFQGP (SEQ ID NO: 444))-alanine linker (A)- residues 24-463 of
GLP1R-linker (PAG)-3C protease recognition site (LEVLFQGP (SEQ ID
NO: 444))-8× Histidine tag (HHHHHHHH (SEQ ID NO: 445)). Compared
to the UniProt entry P43220, our GLP1R sequence also contains a
L260F variation located in intracellular loop 2. The L260F variant was
found in historical versions of the UniProt entry for human GLP1R and
was present in constructs used in previous structural studies.
GNAS (encoding Gαs; reference Uniprot: P63092) - The GNAS
sequence used includes mutations to introduce a “dominant negative”
effect. The mutations are S54N, G226A, E268A, N271K, K274D,
R280K, T284D, and I285T.
GNB1 (encoding Gβ1; reference Uniprot: P62873) - The GBB1
construct was composed as follows: N-terminal methionine (M)-6×
histidine tag (HHHHHH (SEQ ID NO: 441))- linker (GSSG (SEQ ID NO:
446))- residues 2-340 of GBB1.
GNG2 (encoding Gγ2; reference Uniprot: P59768)

For recombinant protein expression, ExpiSf9 Cells (Gibco, A35243) cultured in ExpiSf CD Medium (Thermo Fisher, A3767803) were infected with baculovirus for GLP-1R, GNAS, and the same baculovirus for the expression of both GBB1 and GNG2 in a 3:3:1 ratio, respectively. Cells were cultured at 27° C. and 120 rpm for 3 days. Cells (stored at −80° C.) were thawed and resuspended in buffer comprising 25 mM Tris (pH 7.5) (Invitrogen, 15567-027), 50 mM NaCl (Thermo Fischer, 24740011), 5 mM CaCl2, 2 mM MgCl2, 25 mU/ml Apyrase (Sigma, A6410), and cOmplete, EDTA-free protease inhibitor tablet (Roche, 05056489001). 365 nM REGN9268-M3190 F(ab′) was added to the pellet as it was thawing to promote binding of the G proteins to GLP-1R. The mixture was rotated for 1 hour at room temperature. LMNG (Anatrace, NG310) and CHS (Anatrace, CH210) were added to the mixture to final concentrations of approximately 1% and 0.1%, respectively, and the mixture was rotated for an additional 1 hour at 4° C. Insoluble material was pelleted by ultra-centrifugation (100,000×g, 4° C.) and the supernatant was mixed with ANTI-FLAG M2 affinity agarose gel slurry (Sigma, A2220) at 4° C. for 1 hr. The affinity gel was collected in a gravity column and washed with buffer containing 0.01% LMNG, 0.001% CHS, 25 mM Tris (pH 7.5), 100 mM NaCl, 2 mM MgCl₂, and 5 mM CaCl₂. Protein was eluted with the wash buffer containing 5 mM EGTA and 0.1 mg/ml 3× FLAG peptide without CaCl₂ (Sigma, SAE0194). The eluate was concentrated and purified by SEC using a tandem SEC column configuration; a Superose 6 Increase 10/300 (GE, 29-0915-96) column was directly upstream of a Superdex 200 Increase 10/300 column (Cytiva, 28990944) column in 0.01% LMNG, 0.001% CHS, 25 mM Tris pH 7.5, 100 mM NaCl, and 2 mM MgCl₂. Peak fractions were pooled and concentrated in a 100 kDa MWCO Amicon Ultra-0.5 ml centrifugal filter (UFC510024).

For the purification of the GLP-1R/Gs/REGN15869-M3190 complex, 57 nM REGN15869-M3190 was added during pellet thawing and the pellet resuspension buffer included 50 mU/ml Apyrase.

To prepare ‘untethered’ GLP-1R/Gs/M3190/REGN9268 F(ab′) or GLP-1R/Gs/M3190/REGN15869 samples, GLP-1R/Gs complexes were assembled in the presence of 8 μM or 2 μM M3190-L16 respectively, and 50 mU/ml apyrase. 1.0 or 0.2 μM M3190-L16 was included in the affinity and SEC buffers, respectively. The resulting GLP-1R/Gs/M3190 complexes were incubated with untethered ligand-free REGN9268 F(ab′) or REGN15869 prior to cryoEM grid preparation.

### REGN9268-M3190 F(ab′) Production

REGN9268-M3190 was diluted in 20 mM sodium acetate (pH 5.0) (Avantor, 3470-01) and briefly dialyzed against 20 mM Tris (pH 7.5), 10 mM NaCl at room temperature using a Slide-A-Lyzer G2 Dialysis Cassette, 20K MWCO (Thermo Fisher, 87737). IdeS was added and incubated with the antibody for 30 minutes at 37° C. to produce F(ab′)2. 2-MEA (50 mM) and EDTA (10 mM) were added, and the reduction of F(ab′)2 to F(ab′) took place for 20 minutes at 37° C. The sample was briefly dialyzed using a Slide-A-Lyzer G2 Dialysis Cassette, 20K MWCO (Thermo Fisher, 87738) in prechilled 20 mM Tris (pH 7.5), 10 mM NaCl. After dialysis, 50 mM iodoacetamide was added to alkylate free, hinge-region cysteines and the reaction was carried out at room temperature for 5 minutes. A brief dialysis was carried out using a Slide-A-Lyzer G2 Dialysis Cassette, 20K MWCO (Thermo Fisher, 87738) in 20 mM sodium acetate (pH 5.0) at room temperature and the protein was incubated overnight with 0.5% detergent (LMNG) at 4° C. The protein was purified by SEC using a Superdex 200 Increase 10/300 column coupled with a 0-500 mM NaCl gradient.

### CryoEM Sample Preparation and Data Collection

Purified GLP-1R complexes were applied to UltrAuFoil 0.6/1 300 mesh grids (Quantifoil) that were freshly plasma cleaned using a Solarus II (Gatan), then blotted and plunge-frozen into liquid ethane cooled by liquid nitrogen using a Vitrobot Mark IV (ThermoFisher) operated at 4° C. and 100% humidity. CryoEM data were collected on a Titan Krios G3i microscope (ThermoFisher) equipped with a K3 camera (Gatan) operating in counted mode. Automated data collection was carried out using EPU (ThermoFisher). Movies were collected at a nominal magnification of 105,000× (0.85 Å/pixel) and a requested defocus range of −1.4 to −2.4 μM. The number of movies collected per sample are as follows: GLP-1R/Gs/REGN9268-M3190 Fab (6,882), GLP-1R/Gs/M3190/REGN9268 Fab (6,114), GLP-1R/Gs/REGN15869-M3190 (9,294), GLP-1R/Gs/M3190/REGN15869 (7,129).

### CryoEM Data Processing and Map Generation

CryoEM data were processed using cryoSPARC v2 or RELION 3. Movies were motion-corrected and CTF parameters were estimated for the summed micrographs. Particle coordinates were picked using 2D templates. Particle images corresponding to false positives, contaminants, or broken complexes were removed after multiple rounds of 2D classification. Homogenous subsets of particles images corresponding to the target complexes were obtained after multiple rounds of 3D classification. In the case of the GLP-1R/Gs/M3190/REGN9268 Fab sample, focused refinement of the GLP-1R/M3190/REGN9268 Fab was conducted to improve the resolution of the REGN9268/GLP-1R contact region. The resolutions (calculated using FSC=0.143) of the maps used for model building for each complex are as follows: GLP-1R/Gs/REGN9268-M3190 Fab (4.3 Å), GLP-1R/Gs/M3190/REGN9268 Fab (3.9 Å), GLP-1R/Gs/REGN15869-M3190 Fab (3.5 Å), GLP-1R/Gs/M3190/REGN15869 Fab (3.3 Å).

### Model Building and Refinement

Initial models were obtained from published GLP-1R complex structures (PDB IDs 5NX2 and 6X18), as well as homology models for Fab fragments generated from previous REGN structures. The Fit-in-map function of UCSF Chimera was used to dock initial models into their corresponding densities. Manual model building was carried out using Coot version 0.8.9, and real space refinements were conducted in Phenix version 1.19. Restraints for the M3190 ligand were obtained using the eLBOW program in Phenix.

### CryoEM Structure of REGN9268-M3190 Bound to GLP-1R/Gs

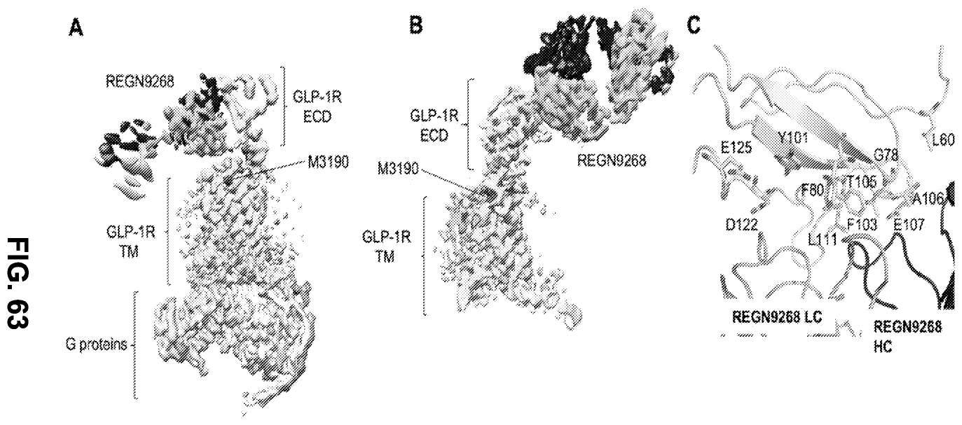

A 4.3 Å resolution reconstruction of REGN9268-M3190 bound to GLP-1R/Gs was obtained by cryoEM (FIG. 63A). CryoEM density corresponding to the GLP-1R transmembrane (TM) domain, M3190 ligand, and Gs proteins displayed most bulky side chains, permitting model building and refinement. However, the GLP-1R extracellular domain (ECD) and REGN9268 Fab were resolved to lower resolution, sufficient for docking models of individual domains. The PEG linker and LLQGSG tag (SEQ ID NO: 18) at the light chain REGN9268 N-terminus were not resolved due to flexibility.

The cryoEM structure shows that the GLP-1R TM domain and Gs adopt a conformation consistent with published structures of active-state GLP-1R/Gs complexes bound to peptide and non-peptide agonists. In this conformation, Gαs helix 5 inserts into a cytoplasmic-facing cavity formed by GLP-1R TM helices 2,3,5,6,7. Additional contacts between Gαs N helix and GLP-1R TM4, and between Gβ and GLP-1R H8 appear to further stabilize the association between the receptor and Gs protein.

The M3190 ligand adopts an overall helical conformation in the GLP-1R binding pocket, with its N-terminus positioned in the TM domain core and its C-terminus pointing extracellularly. The interactions between GLP-1R TM domain and M3190 are similar to those observed in a previously published crystal structure of GLP-1R in complex with ‘peptide 5’. In this published structure (PDB 5NX2), the C-terminal end of ‘peptide 5’ is poised to make contacts with GLP-1R ECD. By contrast, in the current structure of REGN9268-M3190/GLP-1R/Gs complex, apparent contacts between M3190 and GLP-1R ECD are absent due to its displaced ECD position, which is expanded upon below.

REGN9268 Fab binds a GLP-1R ECD epitope that includes part of the two-stranded anti-parallel beta sheet (β3/β4) that resides on the opposite face of the ECD relative to the N-terminal helix. Structural alignment with available GLP-1R/GLP-1 complex structures indicates that REGN9268 binds GLP-1R ECD in an orientation that would not sterically block GLP-1.

Previous structural studies have demonstrated that the GLP-1R ECD is flexibly attached to the TM domain and can adopt various positions depending on the bound ligand. In the cryoEM structure, REGN9268-M3190 appears to stabilize the GLP-1R ECD in a position relative to the TM domain that is distinct from published structures of GLP-1R bound to GLP-1 (PDB 6X18), ‘peptide 5’ (PDB 5NX2), or in the absence of orthosteric ligand (PDB 6LN2). Using the Cα atom of Y42 (which resides toward the center of the N-terminal helix) as a proxy for ECD position, the ECD is rotated and displaced toward the extracellular sides of TMs 1 and 2 by approximately 31, 32, or 35 Å in the REGN9268-M3190 complex relative to PDBs 6X18, 5NX2, and 6LN2, respectively. The displaced position of the ECD places the bound REGN9268 Fab such that its light chain N-terminus is situated adjacent to extracellular surface of the orthosteric pocket, in close proximity to the tethered M3190 ligand.

### CryoEM Structure of REGN9268 Bound to M3190/GLP-1R/Gs

In the REGN9268-M3190 Fab/GLP-1R/Gs cryoEM structure described above, the REGN9268/GLP-1R ECD epitope could not be precisely defined at the amino acid side chain level due to relatively poor resolution in this region. Aiming to better resolve the REGN9268/GLP-1R interface, a cryoEM reconstruction of the REGN9268 Fab/M3190/GLP-1R/Gs complex (in which the Fab and ligand were added separately in ‘untethered’ form) was determined to an overall resolution of 3.9 Å (FIG. 63B). The relatively higher resolution cryoEM structure of the REGN9268 Fab/M3190/GLP-1R/Gs complex was used to assess the epitope-paratope interactions of REGN9268 on GLP-1R (Table 37, FIG. 63C). The REGN9268 epitope is centered around the two-stranded antiparallel p-sheet composed of β3/β4 strands of the ECD. REGN9268 binds in a diagonal orientation with respect to β3/β4, with the heavy chain situated toward the N-terminal side of β3 and the light chain located closer to the N-terminal side of β4. REGN9268 interactions with GLP-1R ECD are mediated by CDRs H1, H3, and L1. CDR-H1 contacts GLP-1R residues L60, A106, and E107. CDR-H3 makes apparent interactions with G78, F103, T105 and A106. CDR-L1 contacts with GLP-1R residues F80, Y101, D122, and E125.

A comparison of the REGN9268-M3190 Fab/GLP-1R/Gs (‘tethered’) and REGN9268 Fab/M3190/GLP-1R/Gs (‘untethered’) structures reveals the potential impact of mAb-ligand tethering on the conformation of the complex. When considering the ECD in isolation, the overall binding mode of REGN9268 Fab to GLP-1R ECD is shared in the REGN9268-M3190 Fab/GLP-1R/Gs and REGN9268 Fab/M3190/GLP-1R/Gs structures, indicating that tethering of the ligand does not grossly alter the ECD epitope of REGN9268. However, the relative positioning of ECD and TM domain are distinct when comparing the two structures; while the ECD domain in the REGN9268 Fab/M3190/GLP-1R/Gs (‘untethered’) complex structure adopts a position similar to that observed in PDB 5NX2, the ECD N-terminal helix is displaced by ˜30 Å relative to the TM domain in the REGN9268-M3190 Fab/GLP-1R/Gs (‘tethered’) cryoEM structure. Concurrent with the ECD displacement, the REGN9268 light chain N-terminus adopts a position adjacent to the orthosteric pocket, presumably facilitating traversal of the linker connecting antibody and ligand. Therefore, the structural data suggests that simultaneous binding of REGN9268 antibody and tethered ligand depends on displacement of the GLP-1R ECD.

### CryoEM Structure of REGN15869-M3190 Bound to GLP-1R/Gs

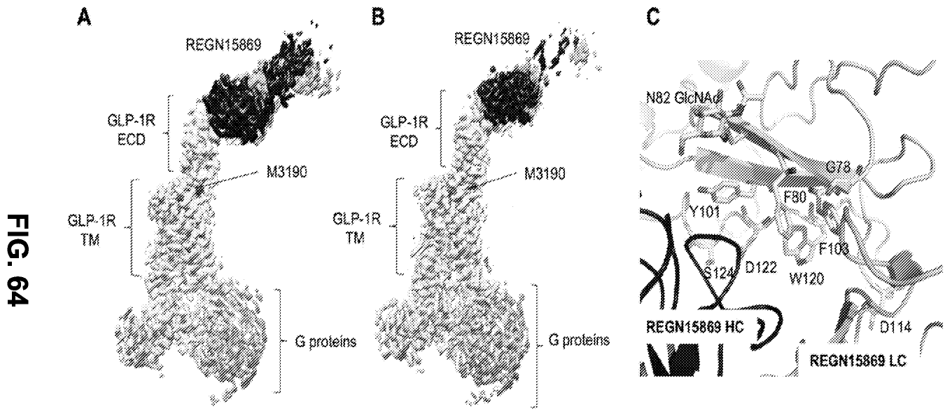

A cryoEM dataset was collected for the REGN15869-M3190/GLP-1R/Gs complex sample. Although the bivalent IgG-based ATL was used to prepare this complex, single REGN15869-M3190 Fab arm complexes with GLP-1R/Gs were processed as individual particles. The resulting 3.5 Å resolution map showed clear densities for most bulky side chains in the REGN15869 variable region, GLP-1R ECD and TM domains, M3190 ligand, and G protein (FIG. 64A). The M3190 PEG linker and N-terminal tag on the light chain were not resolved due to flexibility. The GLP-1R TM domain and G proteins have a similar disposition to those of the REGN9268-M3190 Fab/GLP-1R/Gs structure described above as well as previously described structures of agonist-bound GLP-1R/Gs complexes. The binding pose of the tethered M3190 peptide ligand in the TM domain is analogous to that observed in the REGN9268-M3190 complex structure. In the REGN15869-M3190 complex, the ECD is positioned adjacent to the ligand binding pocket, thereby providing additional contacts with the C-terminal portion of the M3190 peptide.

The relative positions of the GLP-1R ECD and TM domains in the REGN15869-M3190 complex is subtly different from that observed in the ‘peptide-5’-bound GLP-1R structure; the center of the ECD N-terminal helix (as determined by the position of the Ca atom of Y42) is only displaced by ˜4 Å. This ECD position contrasts the displaced ECD conformation observed in the REGN9268-M3190 complex.

The cryoEM structure indicates that the REGN15869 mAb binds GLP-1R at an ECD epitope that includes residues in the β3/β4 anti-parallel β-sheet (Table 38, FIG. 64C). This epitope overlaps significantly with the REGN9268 epitope. However, the binding orientation of REGN15869 is distinct; the REGN15869 heavy chain is positioned toward the C-terminus of β3 and N-terminus of β4 and the light chain is closer to the N- and C-termini of β3 and β4, respectively. Contacts with F80, Y101, W120, D122, and S124 are mediated by CDR-H3. CDR-H1 residue S31 approaches within 4 Å of the GlcNAc moiety covalently linked to N82, which was clearly visible in the cryoEM map. CDR-L1 makes contacts with GLP-1R residues F103 and D114. CDR-L2 contributes additional contacts to F103, as well as an apparent polar interaction with the backbone carbonyl of G78.

### CryoEM Structure of REGN15869 Bound to M3190/GLP-1R/Gs

A cryoEM structure of the ‘untethered’ REGN15869/M3190/GLP-1R/Gs complex was obtained at an overall resolution of 3.3 Å (FIG. 64B). This structure has overall similar features to the ‘tethered’ REGN15869-M3190/GLP-1R/Gs complex structure described above, excepting an ˜8° tilt of the GLP1R ECDs relative to each other. The ECD tilt results in a shorter distance (˜37 Å in the ‘tethered’ complex structure compared to ˜41 Å in the ‘untethered’ complex structure) between the light chain N-terminus (resolved to residue D7 in the cryoEM density) of the bound REGN15869 and the unnatural amino acid residue to which the intervening PEG linker is conjugated. The structural data therefore suggest that a minor shift of ECD is associated with simultaneous binding of REGN15869 antibody and tethered M3190 ligand. This contrasts with REGN9268-M3190, in which a significant displacement of ECD was observed in the cryoEM structure. The different ECD conformations are likely a product of the distinct binding angles of REGN9268 and REGN15869; different geometries are required to position the antibody light chain N-terminus in sufficient proximity to the M3190 ligand such that both can bind simultaneously.

TABLE 37
Summary of REGN9268/GLP-1R contact residues. Contacting residues
are defined as GLP-1R amino acids with non-hydrogen atoms within
4 Å of non-hydrogen atoms of antibody.

| | | Antibody Residue(s) Interacting With | |
|---|---|---|---|
| | GLP-1R | Indicated GLP-1R residue | |
| Antibody | Residue | Heavy Chain | Light Chain |
| REGN9268 | L60 | R31 | |
| | G78 | R104 | |
| | F80 | | Y39 |
| | Y101 | | R38 |
| | F103 | L103 | |
| | T105 | G100, Y101 | |
| | A106 | R31, G100 | |
| | E107 | R31, Y32 | |
| | L111 | L103 | |
| | D122 | | R38 |
| | E125 | | R38 |

TABLE 38
Summary of REGN15869/GLP-1R contact residues. Contacting residues
are defined as GLP-1R amino acids with non-hydrogen atoms within
4 Å of non-hydrogen atoms of antibody.

| | | Antibody Residue(s) Interacting | |
|---|---|---|---|
| | GLP-1R | With Indicated GLP-1R residue | |
| Antibody | Residue | Heavy Chain | Light Chain |
| REGN15869 | G78 | | Y55 |
| | F80 | L100, I101 | |
| | N82(GlcNAc) | S31 | |
| | Y101 | A102, P103 | |
| | F103 | | W38, A56 |
| | D114 | | N36 |
| | W120 | M106 | |
| | D122 | P103 | |
| | S124 | P103 | |

### REFERENCES FOR EXAMPLES 23

1. Liang, Y. L. et al. Phase-plate cryo-EM structure of a biased agonist-bound human GLP-1 receptor-Gs complex. Nature 555, 121-125 (2018).
2. Johnson, R. M. et al. Cryo-EM structure of the dual incretin receptor agonist, peptide-19, in complex with the glucagon-like peptide-1 receptor. Biochem Biophys Res Commun 578, 84-90 (2021).
3. Cong, Z. et al. Structural basis of peptidomimetic agonism revealed by small-molecule GLP-1R agonists Boc5 and WB4-24. Proc Natl Acad Sci USA 119, e2200155119 (2022).
4. Liang, Y. L. et al. Dominant Negative G Proteins Enhance Formation and Purification of Agonist-GPCR-G Protein Complexes for Structure Determination. ACS Pharmacol Transl Sci 1, 12-20 (2018).
5. Punjani, A., Rubinstein, J. L., Fleet, D. J. & Brubaker, M. A. cryoSPARC: algorithms for rapid unsupervised cryo-EM structure determination. Nat Methods 14, 290-296 (2017).
6. Zivanov, J. et al. New tools for automated high-resolution cryo-EM structure determination in RELION-3. Elife 7(2018).
7. Emsley, P., Lohkamp, B., Scott, W. G. & Cowtan, K. Features and development of Coot. Acta Crystallogr D Biol Crystallogr 66, 486-501 (2010).
8. Afonine, P. V. et al. Real-space refinement in PHENIX for cryo-EM and crystallography. Acta Crystallogr D Struct Biol 74, 531-544 (2018).
9. Zhang, Y. et al. Cryo-EM structure of the activated GLP-1 receptor in complex with a G protein. Nature 546, 248-253 (2017).
10. Kawai, T. et al. Structural basis for GLP-1 receptor activation by LY3502970, an orally active nonpeptide agonist. Proc Natl Acad Sci USA 117, 29959-29967 (2020).
11. Zhang, X. et al. Differential GLP-1R Binding and Activation by Peptide and Non-peptide Agonists. Mol Cell 80, 485-500 e7 (2020).
12. Jazayeri, A. et al. Crystal structure of the GLP-1 receptor bound to a peptide agonist. Nature 546, 254-258 (2017).
13. Underwood, C. R. et al. Crystal structure of glucagon-like peptide-1 in complex with the extracellular domain of the glucagon-like peptide-1 receptor. J Biol Chem 285, 723-30 (2010).
14. Wu, F. et al. Full-length human GLP-1 receptor structure without orthosteric ligands. Nat Commun 11, 1272 (2020).

### Examples 24. Cell-Based cAMP Responsive Luciferase Reporter Assay

Glucagon-like peptide 1 receptor, GLP1R, is a member of the secretin family (Class B) of G protein-coupled receptors (GPCRs). Upon binding of its ligand, GLP-1, GLP1R initiates a downstream signaling cascade through Gas G-proteins that raises intracellular cyclic AMP (cAMP) levels, which leads to the transcriptional regulation of genes (Donnelly 2012). GLP-1 binding also results in b-arrestin 1 and b-arrestin 2 recruitment to GLP1R.

To test the activity of GLP1R agonist payloads, GLP1R agonist linker-payloads (LPs), and anti-GLP1R antibody-tethered ligands (ATLs) of the invention, a cell-based cAMP responsive luciferase reporter assay was developed. To generate the assay cell line, the firefly luciferase gene was placed under the control of a cAMP response element (CRE) located upstream of a minimal promoter and transfected into HEK293 cells and referred to herein as HEK293/CRE-Luc cells. HEK293/CRE-Luc cells were then engineered to express full-length human GLP1R (HEK293/CRE-Luc/hGLP1R), cynomolgus GLP1R (HEK293/CRE-Luc/mfGLP1R), or mouse GLP1R (HEK293/CRE-Luc/mGLP1R).

ATL and mAb binding to cells was assessed by flow cytometry. Briefly, 95,000-540,000 cells were suspended in staining buffer (PBS+2% FBS+0.2% sodium azide) in 96-well, V-bottom plates (Axygen, #P-96-450V-C-S) and incubated for 30 minutes at 4° C. with serial dilutions of the anti-GLP1R ATLs, non-binding control ATL, or unconjugated antibodies. The cells were then washed once with staining buffer and then incubated with an APC conjugated Fab′2 anti-human heavy+light IgG secondary antibody (Jackson Immunoresearch, #109-136-170) at 5 ug/mL for 30 minutes at 4° C. Cells were then washed once and stained with a cell viability dye (Live/dead Fixable Green Dead Cell Stain Kit for 488 nm excitation, Molecular Probes, #L34970) at 1× in PBS for 20 minutes at 4° C. Cells were washed once in staining buffer, fixed for 20 minutes at 4° C. using a 50% solution of Cytofix (BD Biosciences, #554655) diluted in PBS, and washed again in staining buffer. Samples were filtered through 96-well filter plate (Pall Laboratory, #8027) and collected in 96-well, U-bottom plate (Falcon, #351177). Samples were run on the iQue Screener PLUS cytometer (IntelliCyt) and results were analyzed using Forecyte analysis software (IntelliCyt) to calculate the geometric mean fluorescence intensity (MFI) after gating for live cells. The last well in each serial dilution series served as a blank control containing only secondary antibody and was plotted as a continuation of the serial dilution. EC₅₀ values were determined using a four-parameter logistic equation over an 8- or 12-point dose response curve (GraphPad Prism), and the signal to noise (S/N) was determined by taking the ratio of the highest MFI on the dose response curve to the MFI in the secondary alone wells.

For the CRE-Luc assay, cells were seeded into 96-well plates (Thermo Fisher Scientific, #136102) at 10,000 cells/well in assay media (Opti-MEM, 0.1% BSA, 1× Penicillin-Streptomycin-Glutamine) and incubated overnight at 37° C. Serial dilutions of the anti-GLP1R ATLs, non-binding control ATL, and unconjugated antibodies were performed in assay media. Serial dilutions of free payload and linker payloads (LP) were performed in 100% DMSO (ATCC, #4-X-5), followed by 1:100 dilution in assay media. Test articles were added to cells (1:5 dilution) with the last well in each serial dilution series serving as a blank control containing only assay media for antibodies and ATLs or assay media with 0.2% DMSO for payloads and LPs. After a 5-hour incubation at 37° C., luciferase activity was determined by addition of ONE-Glo reagent (Promega, #E6130) followed by measurement of relative light units (RLUs) on an EnVision Plate Reader (Perkin Elmer).

Another cell based assay was employed to examine the effects of GLP1R agonist payloads, GLP1R agonist linker-payloads (LPs), anti-GLP1R antibody-tethered ligands (ATLs), and unconjugated anti-GLP1R antibodies of the invention on b-arrestin 2 recruitment. The b-arrestin 2 recruitment assay was performed following manufacter's instructions (Eurofins DiscoverX, #93-0300E2CP0M), with minor modifications. Briefly, PathHunter eXpress GLP1R CHO-K1 b-Arrestin GPCR (CHO/b-arrestin 2/hGLP1R) cells were thawed and seeded into 96-well plates (Thermo Fisher Scientific, #136102) at 10,000 cells/well in Cell Plating Reagent (Eurofins DiscoverX, #93-0300E2CP0M) and incubated for 48 hours at 37° C. Serial dilutions of the anti-GLP1R ATLs, non-binding control ATL, and unconjugated antibodies were performed in assay media (Opti-MEM, 0.1% BSA, 1× Penicillin-Streptomycin-Glutamine). Serial dilutions of free payload and LP were performed in 100% DMSO, followed by 1:100 dilution in assay media. Test articles were added to cells (1:5 dilution) with the last well in each serial dilution series serving as a blank control containing only assay media for antibodies and ATLs or assay media with 0.2% DMSO for payload and LP. After a 90-minute incubation at 37° C., PathHunter Detection Reagent (Eurofins DiscoverX, #93-0300E2CP0M) was added to the wells followed by a 1-hour incubation at room temperature. RLUs were measured on EnVision Plate Reader (Perkin Elmer).

EC₅₀ values for luminescence assays were determined using a four-parameter logistic equation over a 12-point dose response curve (GraphPad Prism). The maximum signal relative to GLP-1 (Eₘₐₓ (% GLP-1)) was calculated by the ratio of the highest RLU on the dose response for the test article to the highest RLU on the dose response curve for GLP-1, followed by multiplication by 100.

GLP1R agonist linker payload (M3190) was conjugated to anti-hGLP1R antibodies via N-terminal light chain glutamine tag (Q tag). Several resulting anti-GLP1R ATLs and their unconjugated parental antibodies were tested for cell surface binding to human GLP1R (HEK293/CRE-Luc/hGLP1R), cynomolgus GLP1R (HEK293/CRE-Luc/mfGLP1R), and mouse GLP1R (HEK293/CRE-Luc/mGLP1R) expressing cells via flow cytometry. The parental cell line that lacks GLP1R expression (HEK293/CRE-Luc) was used as a control.

As shown in Table 39, the tested anti-GLP1R antibodies and anti-GLP1R ATLs bound HEK293/HRE-Luc/hGLP1R cells with EC₅₀ values ranging from 3.33 nM to 76.6 nM and S/N values from 106× to 272×. All tested antibodies bound similarly to cynomolgus GLPR1 expressing cells (HEK293/ERE-Luc/mfGLP1R) with EC₅₀ values ranging from 4.13 nM to 135 nM and S/N values from 133× to 556×. All tested antibodies bound mouse GLPR1 expressing cells (HEK293/GRE-Luc/mGLP1R) with EC₅₀ values ranging from 43.1 nM to 772 nM and S/N values from 53× to 154×. Non-binding isotype control antibody and respective ATL bound weakly to all cell lines with EC₅₀ values>1 mM and S/N values <38×. All anti-GLP1R antibodies and anti-GLP1R ATLs bound weakly to parental HEK293/ERE-Luc cells with EC₅₀ values>1 mM and S/N values≤22×. Data was generated across two independent experiments (A and B).

TABLE 39
Anti-GLP1R antibody and anti-GLP1R ATLs Binding to HEK293/CRE-Luc/GLP1R cells.

| | | | HEK293/CRE- | | HEK293/CRE- | | HEK293/CRE- | | HEK293/ | | |
|---|---|---|---|---|---|---|---|---|---|---|---|
| | | | Luc/hGLP1R | | Luc/mfGLP1R | | Luc/mGLP1R | | CRE-Luc | | |
| | | | Flow | Flow | Flow | Flow | Flow | Flow | Flow | Flow | |
| mAb | Q tag | LP | EC50 | S/N | EC50 | S/N | EC50 | S/N | EC50 | S/N | Experiment |
| REGN15869 | None | None | 9.56E−09 | 105.8 | 8.96E−09 | 132.5 | 4.31E−08 | 53.3 | >1E−06 | 8.1 | B |
| REGN15869 | VL N- | M3190 | 7.66E−08 | 106.6 | 1.35E−07 | 172.8 | 7.72E−07 | 62.6 | >1E−06 | 19.1 | B |
| | term | | | | | | | | | | |
| REGN7990 | None | None | 8.48E−09 | 260.2 | 7.47E−09 | 527.5 | 6.14E−08 | 154.0 | >1E−07 | 3.6 | A |
| REGN7990 | VL N- | M3190 | 3.05E−08 | 230.9 | 2.99E−08 | 485.7 | 9.15E−08 | 29.3 | >1E−07 | 6.3 | A |
| | term | | | | | | | | | | |
| REGN9268 | None | None | 3.33E−09 | 272.4 | 4.13E−09 | 555.5 | 4.50E−08 | 137.8 | >1E−07 | 5.9 | A |
| REGN9268 | VL N- | M3190 | 2.49E−08 | 242.3 | 1.89E−08 | 425.9 | 9.20E−08 | 133.2 | >1E−07 | 22.0 | A |
| | term | | | | | | | | | | |
| Isotype | None | None | >1E−06 | 6.1 | >1E−06 | 3.6 | >1E−06 | 3.0 | >1E−06 | 3.3 | B |
| control | | | | | | | | | | | |
| Isotype | VL N- | M3190 | >1E−06 | 37.7 | >1E−06 | 20.7 | >1E−06 | 16.3 | >1E−06 | 18.6 | B |
| control | term | | | | | | | | | | |

> = EC₅₀ values could not be determined with accuracy because the binding did not reach saturation within the tested antibody concentration range. EC₅₀ is reported as greater than the highest tested concentration.

GLP1R activation by ligand binding promotes a signaling cascade through Gαs G-proteins that raises intracellular cyclic AMP (cAMP) levels. In addition, activated GLP1R can recruit b-arrestins, leading to receptor internalization and/or additional signaling pathways (Jones 2022). Ligands can induce disctinct cellular outcomes through the same receptor by preferential signaling through G-proteins or b-arrestins—a phenomenon designated biased signaling.

The anti-GLP1R ATLs were tested for biased signaling by evaluating activity in a cAMP reporter assay (using the HEK293/CRE-Luc/hGLP1R cells) and a b-arrestin 2 recruitment assay (using PathHunter eXpress GLP1R CHO-K1 b-Arrestin GPCR Assay). As shown in Table 40, the three tested anti-GLP1R ATLs increased CRE-dependent luciferase reporter activity in HEK293/CRE-Luc/hGLP1R cells with similar potency (EC₅₀ values ranging from 154 pM to 236 pM) and maximum signal (Eₘₐₓ) relative to GLP-1 ranging from 87.1% to 96.2%. However, the maximum level of b-arrestin 2 recruitment assessed in CHO/b-arrestin 2/hGLP1R cells was reduced for REGN9268 ATL (Eₘₐₓ of 30.7%) when compared to REGN15869 and REGN7990 ATLs (Eₘₐₓ of 110.9% and 103.2%, respectively), even though EC₅₀ values were similar for the three ATLs (ranging from 9.58 nM to 17.2 nM). These results show that different anti-hGLP1R antibodies conjugated to the same GLP1R agonist LP can differentially affect downstream signaling.

The endogenous GLP1R ligand, GLP-1 (7-36) amide (referred to as GLP-1), increased CRE-dependent luciferase reporter activity in HEK293/CRE-Luc/hGLP1R cells with an EC₅₀ value of 67.2 pM and recruited b-arrestin 2 in CHO/b-arrestin 2/hGLP1R cells with an EC₅₀ value of 7.29 nM (Table 40). The payload (M2361) and linker-payload (M3190) increased CRE-dependent luciferase reporter activity in HEK293/CRE-Luc/hGLP1R cells with EC₅₀ values of 22.2 pM and 16.8 pM, respectively, and recruited b-arrestin 2 in CHO/b-arrestin 2/hGLP1R cells with EC₅₀ values of 6.67 nM and 2.48 nM, respectively (Table 40).

TABLE 40
CRE-Dependent Reporter Activity and Beta-Arrestin2 Recruitment
by anti-GLP1R ATLs and GLP1R agonists in HEK293/CRE-Luc/hGLP1R
cells and CHO/b-arrestin 2/hGLP1R cells.

| | | | HEK293/CRE- | | CHO/b-arrestin | |
|---|---|---|---|---|---|---|
| | | | Luc/hGLP1R | | 2/hGLP1R | |
| | | | | Eₘₐₓ | | Eₘₐₓ |
| Test article | Q tag | LP | EC50 | (% GLP-1) | EC50 | (% GLP-1) |
| REGN15869 | None | None | >1E−06 | 0.5 | >1E−06 | 4.3 |
| REGN15869 | VL N- | M3190 | 1.54E−10 | 96.2 | 9.58E−09 | 110.9 |
| | term | | | | | |
| REGN7990 | VL N- | M3190 | 2.36E−10 | 90.9 | 1.72E−08 | 103.2 |
| | term | | | | | |
| REGN9268 | VL N- | M3190 | 1.87E−10 | 87.1 | 1.43E−08 | 30.7 |
| | term | | | | | |
| Isotype control | VL N- | M3190 | 1.35E−08 | 88.0 | >3E−06 | 64.7 |
| | term | | | | | |
| M2361 | None | None | 2.22E−11 | 95.8 | 6.67E−09 | 107.1 |
| M3190 | None | M3190 | 1.68E−11 | 93.9 | 2.48E−09 | 107.7 |
| GLP-1 (7-36) | None | None | 6.72E−11 | 100.0 | 7.29E−09 | 100.0 |
| amide | | | | | | |

> = EC₅₀ values could not be determined with accuracy because luminescence signal did not reach saturation within the tested concentration range. EC₅₀ is reported as greater than the highest tested concentration.

### REFERENCES FOR EXAMPLE 24

1. Donnelly D, The structure and function of the glucagon-like peptide-1 receptor and its ligands, British Journal of Pharmacology, 2012: 166:27-41, PMID 21950636.
2. Jones B, The therapeutic potential of GLP-1 receptor biased agonism, British Journal of Pharmacology, 2022: 179:492-510, PMID 33880754.

### ***

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1.-132. (canceled)

133. An antibody-tethered drug conjugate having a structure of Formula (A):
BA-(L-P)ₘ (A),
wherein:
BA is an antibody or an antigen-binding fragment thereof that specifically binds to glucagon-like peptide-1 receptor (GLP1R) that:
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391 or 411; or a variant thereof;
and/or
a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof;
(ii) is an antibody or antigen-binding fragment thereof that competes for binding to GLP1R with said antibody or antigen-binding fragment of (i); and/or
(iii) is an antibody or antigen-binding fragment thereof that binds to the same epitope of GLP1R as said antibody or antigen-binding fragment of (i);
L is a non-cleavable linker;
optionally, wherein the heavy chain immunoglobulin of the BA does not comprise a C-terminal lysine or lysine and glycine;
optionally, wherein the light chain immunoglobulin and/or heavy chain immunoglobulin of BA comprises an N-terminal Qtag which is conjugated to the non-cleavable linker;
P is a payload having the structure selected from the group consisting of (SEQ ID NOS 448-450, respectively, in order of appearance): 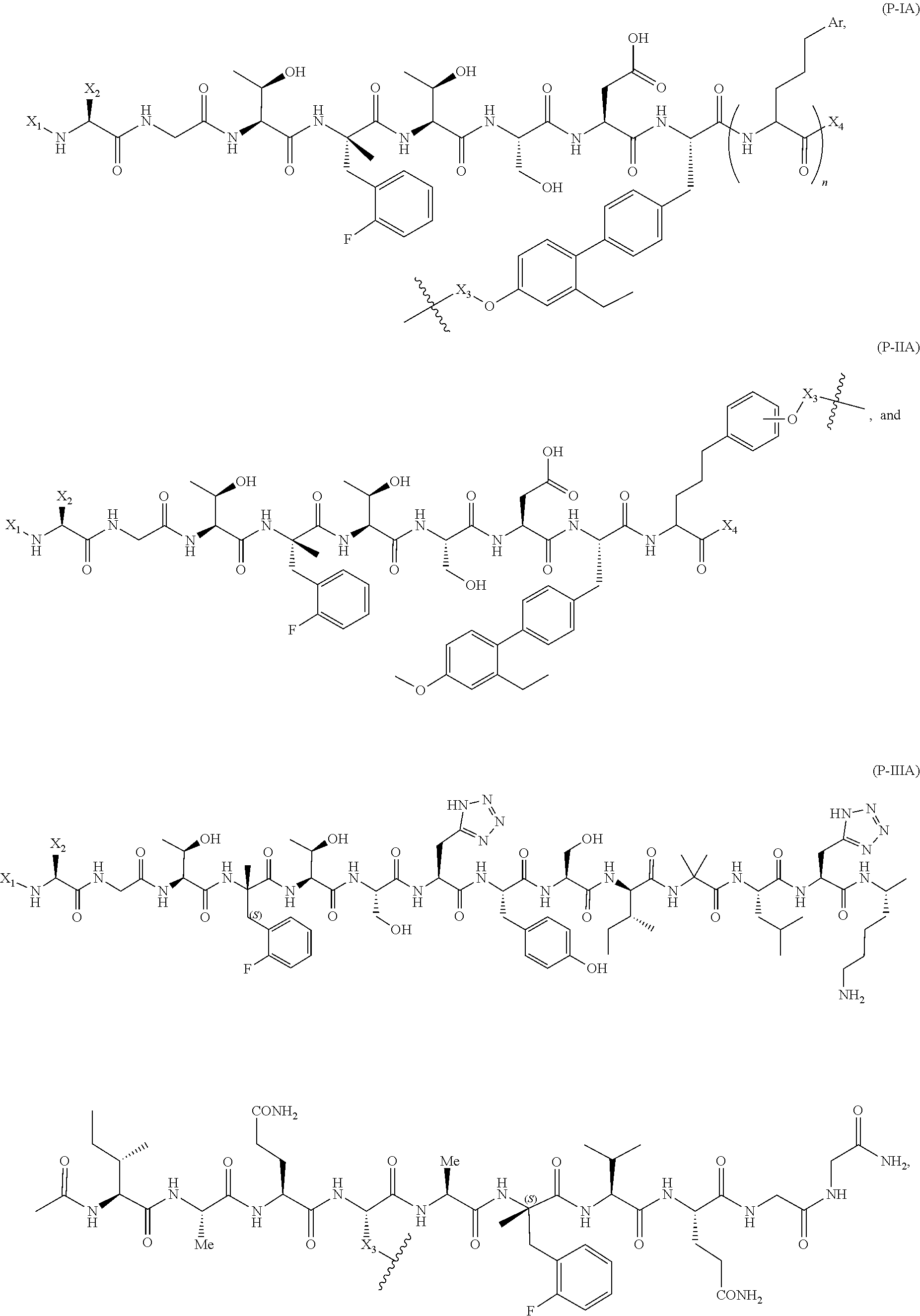
wherein 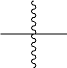
is the point of attachment of the payload to L;
X₁ is selected from H; 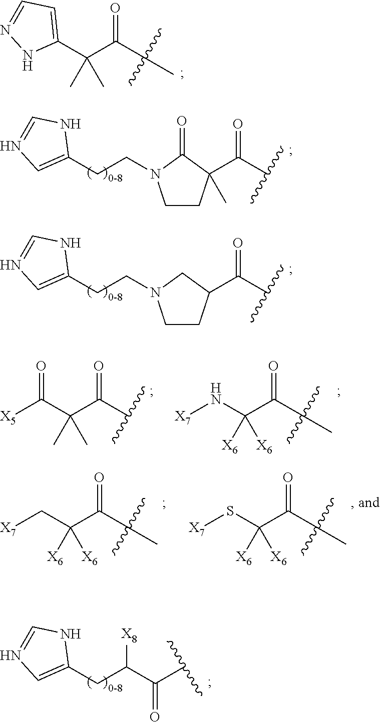
X₂ is selected from 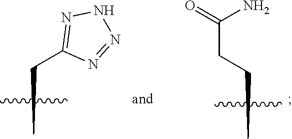
X₃ is selected from a bond, —(CH₂)₂₋₆—NH—, —(CH₂)₂₋₆-Tr-, and —(CH₂)₂₋₆-Tr-(CH₂)₁₋₆—NH, where Tr is a triazole moiety;
n is 0 or 1;
X₄ is selected from —NH₂, —OH and —N(H)(phenyl);
X₅ is selected from —OH, —NH₂, —NH—OH, and 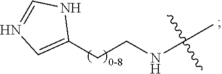
X₆ is independently at each occurrence selected from H, —OH, —CH₃, and —CH₂OH;
X₇ is selected from H, 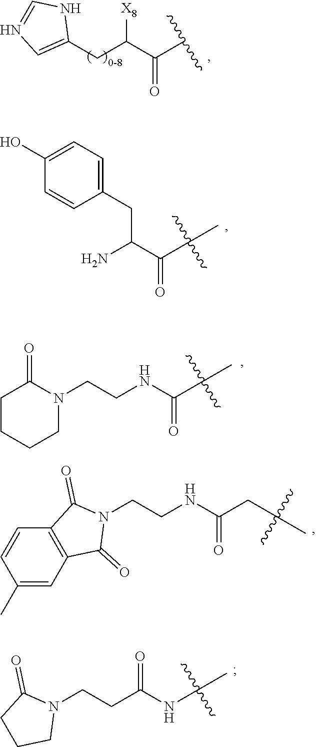
X₈ is selected from H, —OH, —NH₂, and 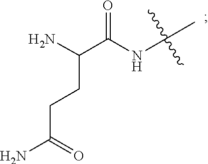 [SMILES: *NC(=O)C(N)CCC(N)=O]
Ar is selected from 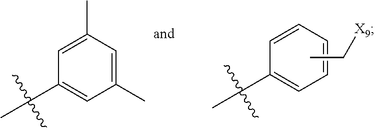
X₉ is selected from —NH₂, 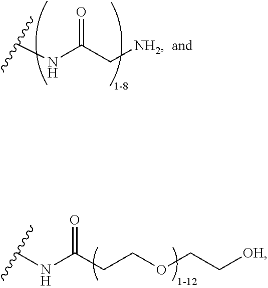
and
m is an integer from 1 to 4,
or a pharmaceutically acceptable salt thereof.

134. The antibody-tethered drug conjugate of claim 133 having a structure of Formula (I):
BA-L-P (I),
wherein:
P is a payload having the structure selected from the group consisting of (SEQ ID NOS 451-452, respectively, in order of appearance): 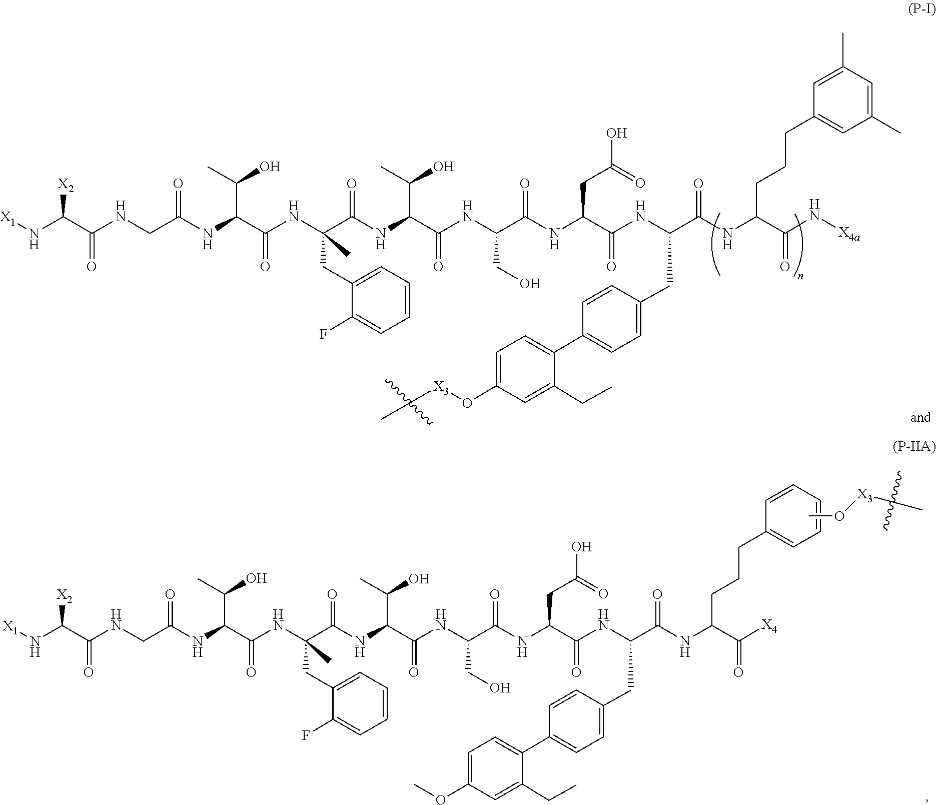
wherein 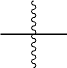
is the point of attachment of the payload to L;
X₁ is selected from H; 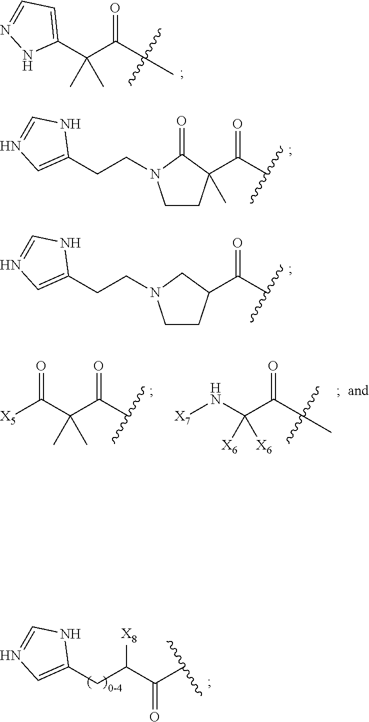
X₂ is selected from 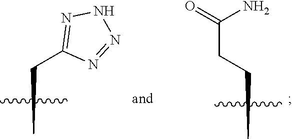
X₃ is selected from —(CH₂)₂₋₆—NH— and —(CH₂)₂₋₆-Tr-, where Tr is a triazole moiety;
n is 0 or 1;
X₄ₐ is selected from H and phenyl;
X₅ is selected from —OH, —NH₂, —NH—OH, and 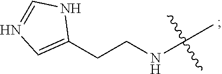 [SMILES: *NCCc1cnc[nH]1]
X₆ is independently at each occurrence selected from H, —OH, —CH₃, and —CH₂OH;
X₇ is selected from H, 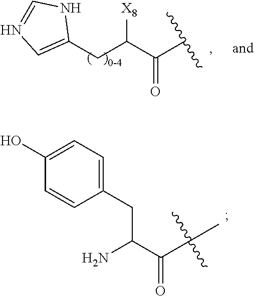
X₈ is selected from H, —OH, —NH₂, and 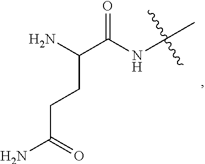 [SMILES: *NC(=O)C(N)CCC(N)=O]
optionally, wherein the light chain immunoglobulin comprises an N-terminal Qtag, or a pharmaceutically acceptable salt thereof.

135. The antibody-tethered drug conjugate of claim 133, wherein P is a payload having the structure selected from the group consisting of (SEQ ID NOS 465, 576, 466-495, 610, 496-497, 611, 498-505, respectively, in order of appearance): 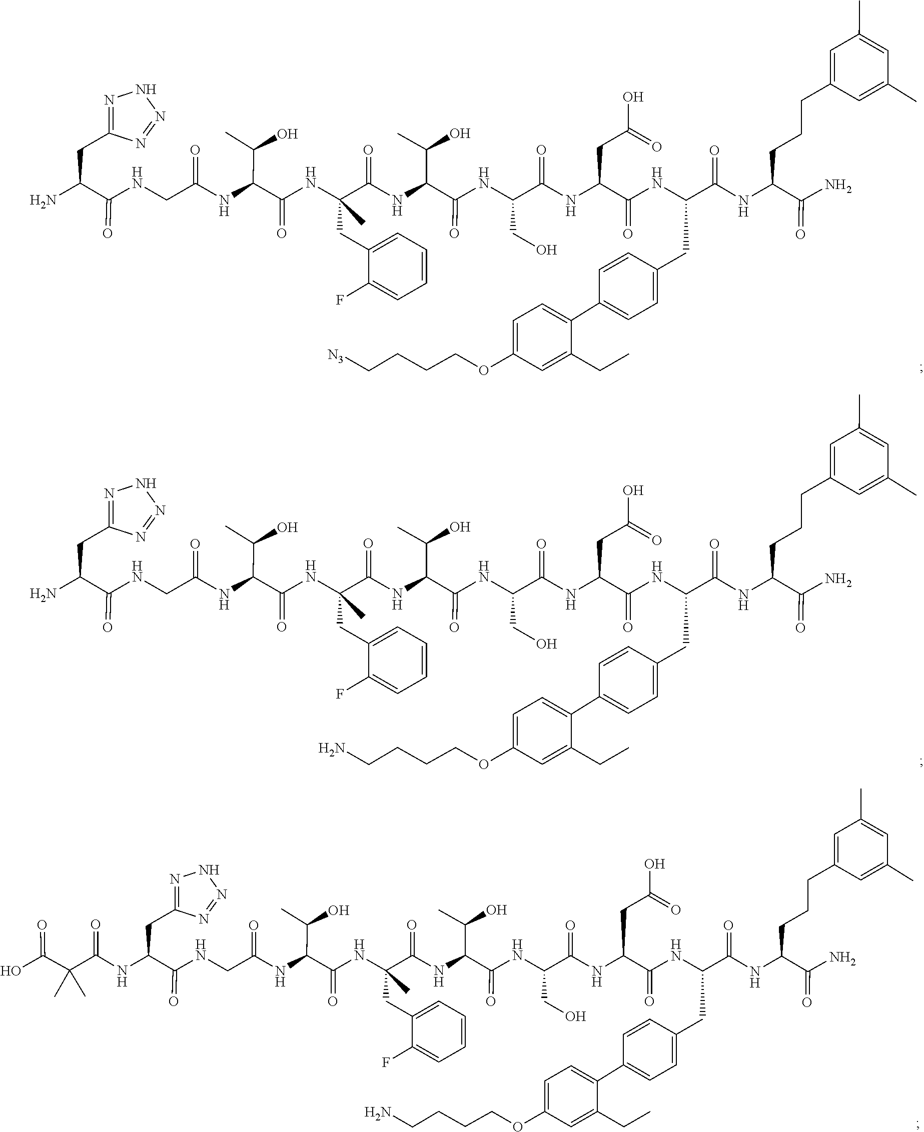 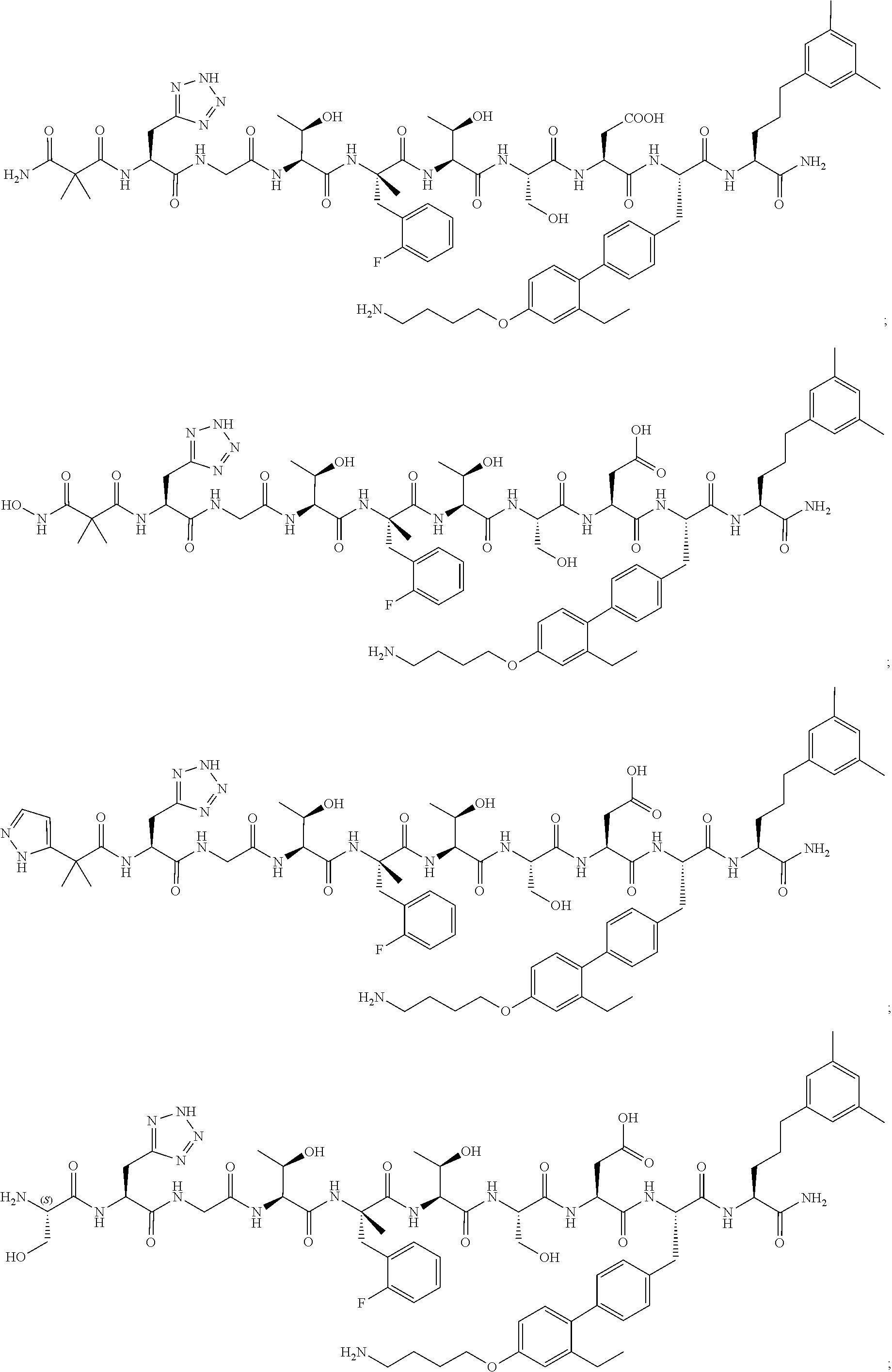 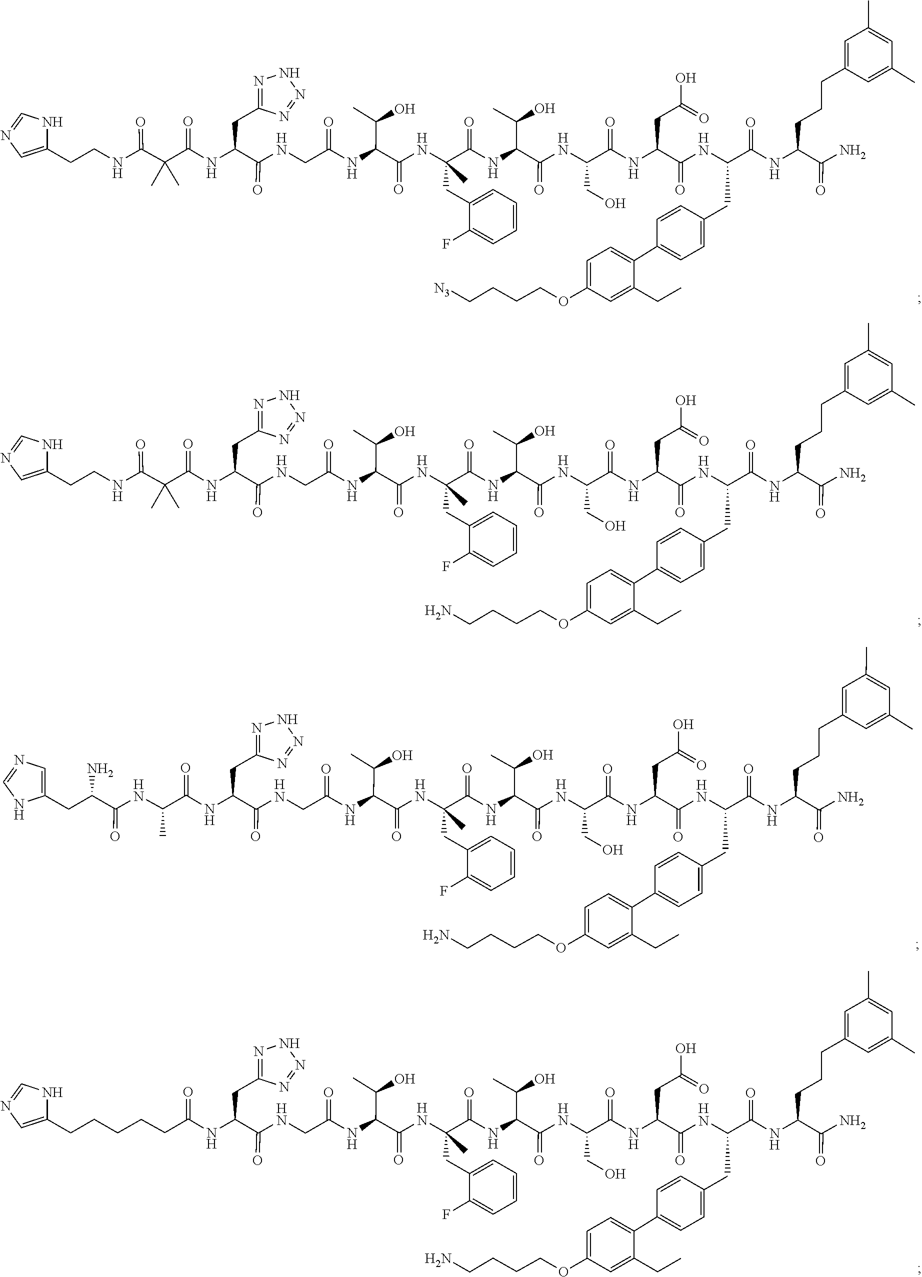 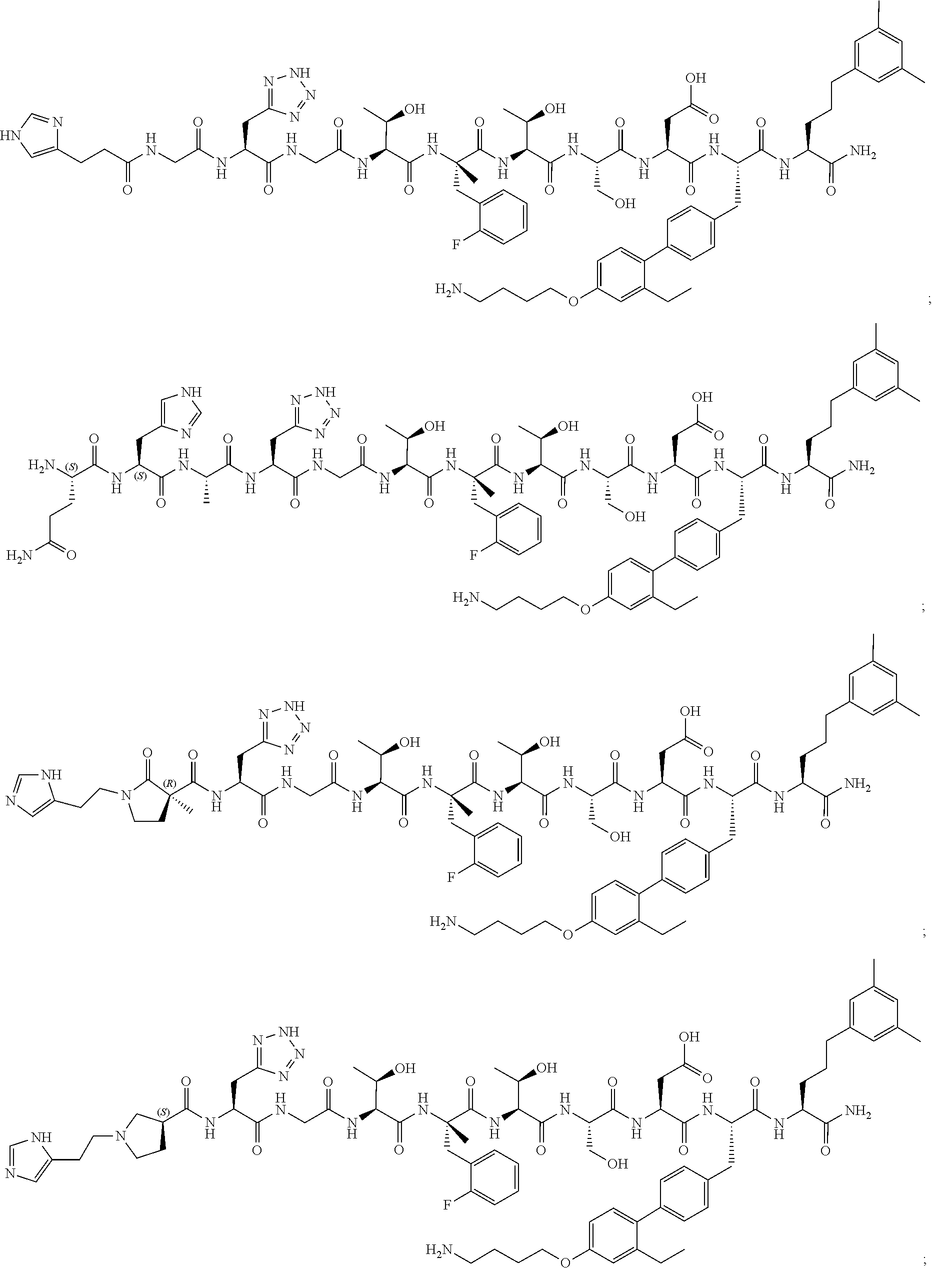 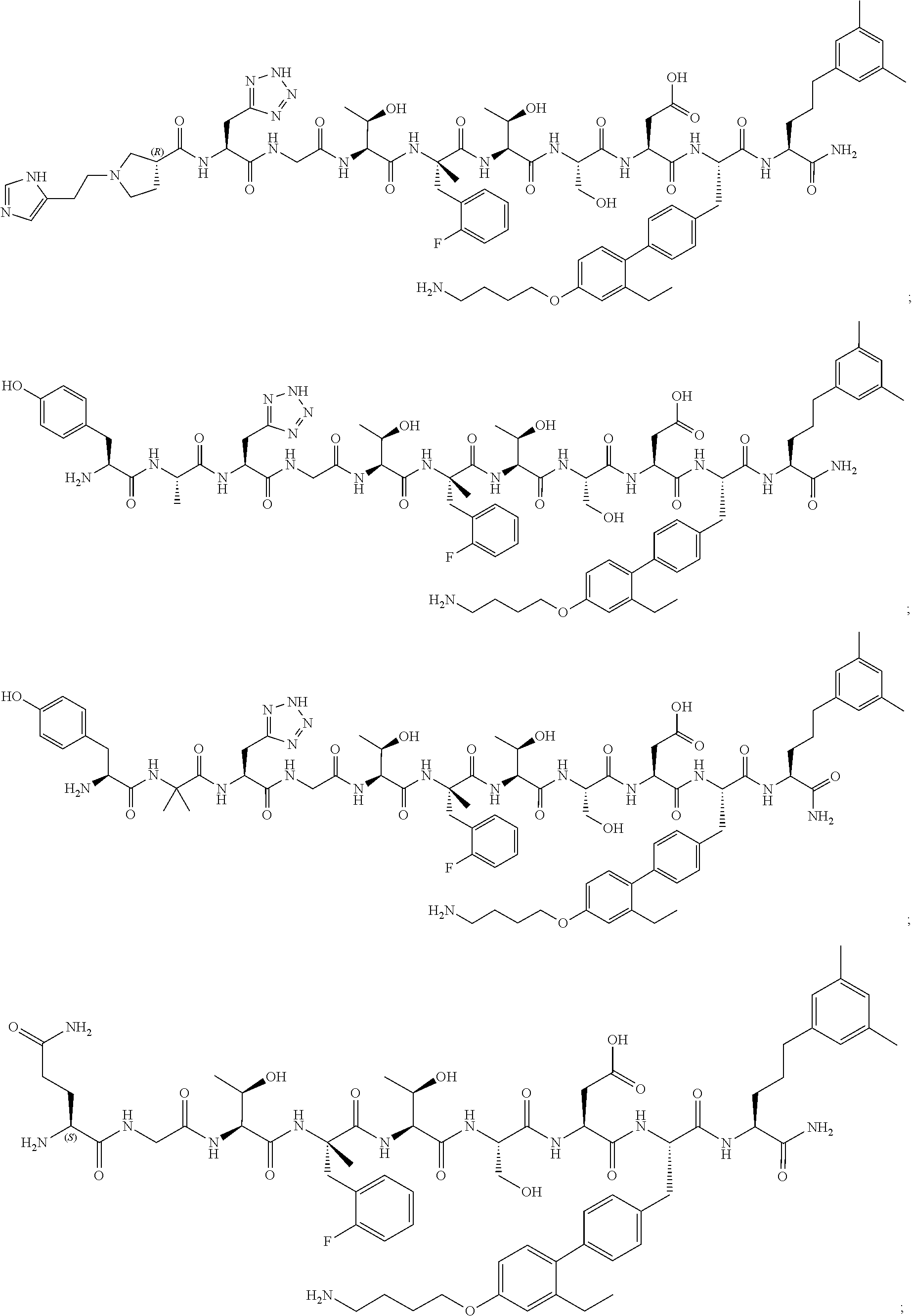 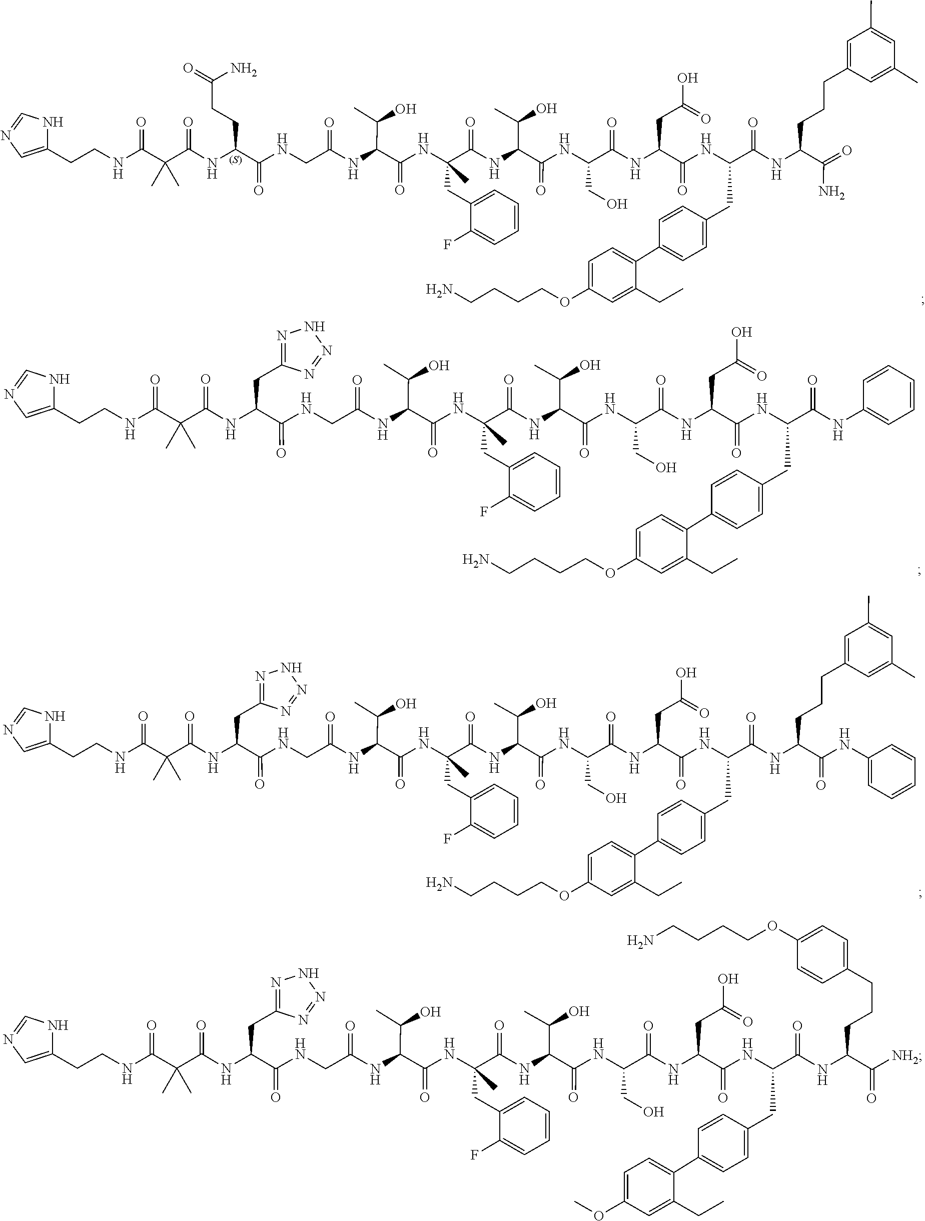 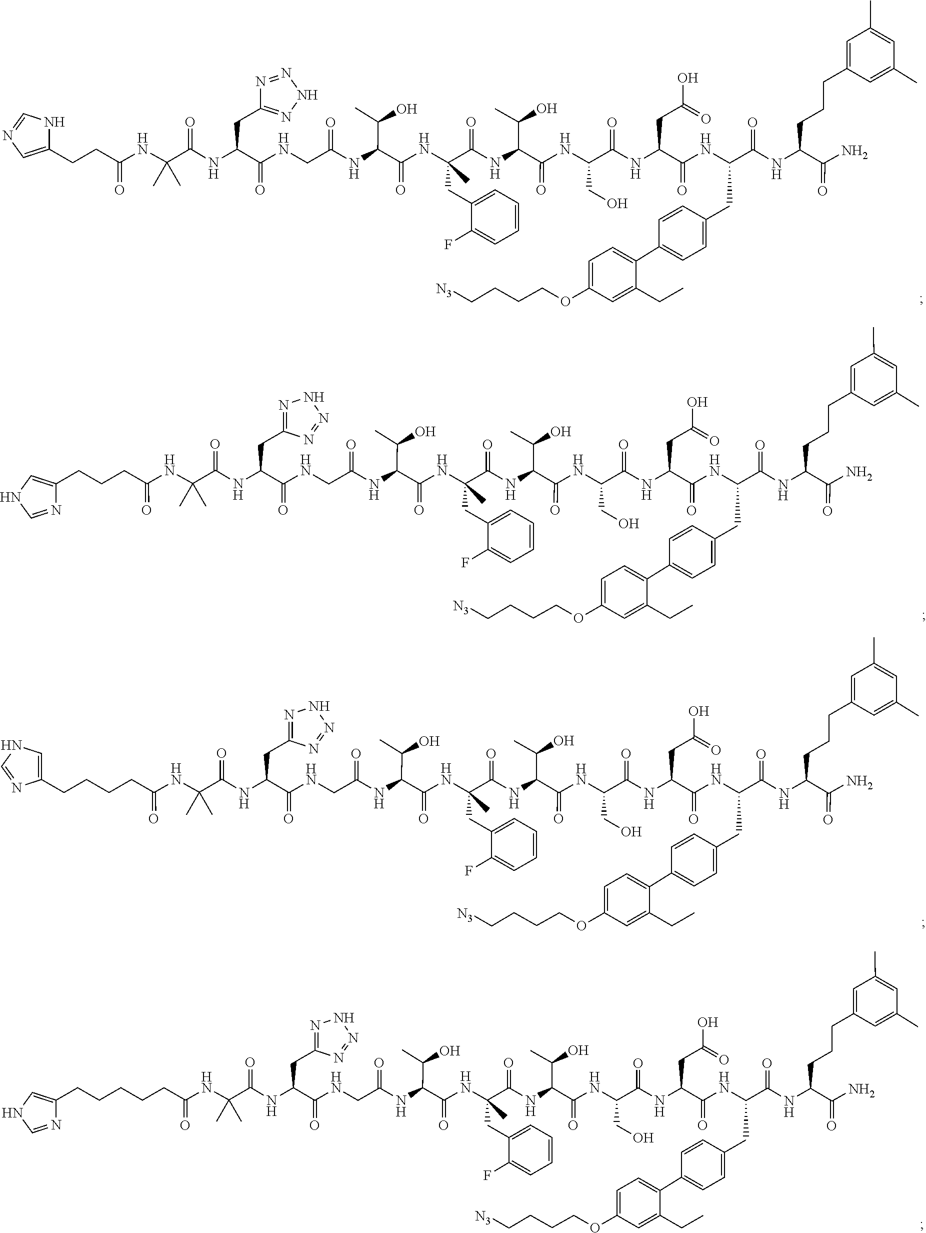 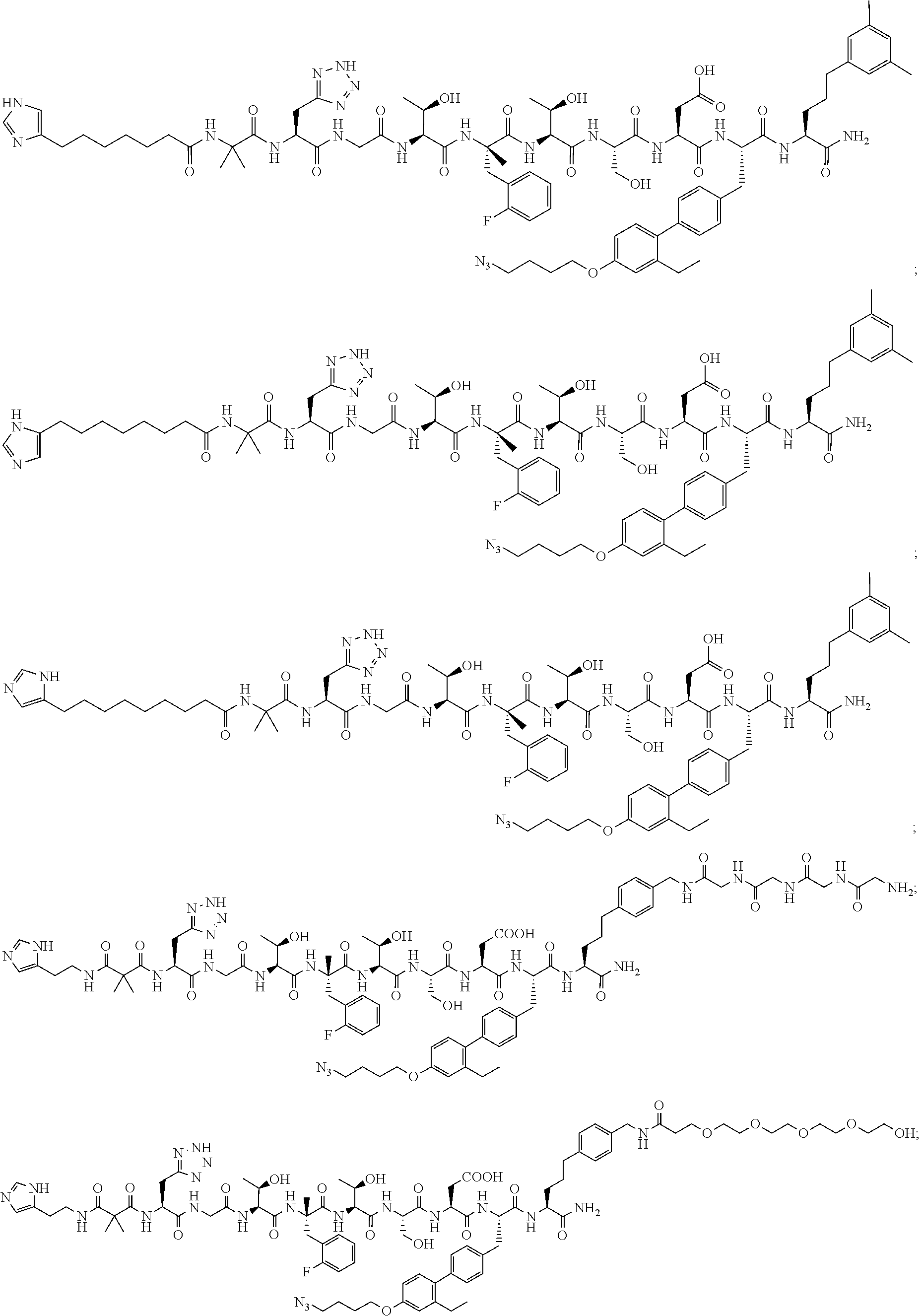 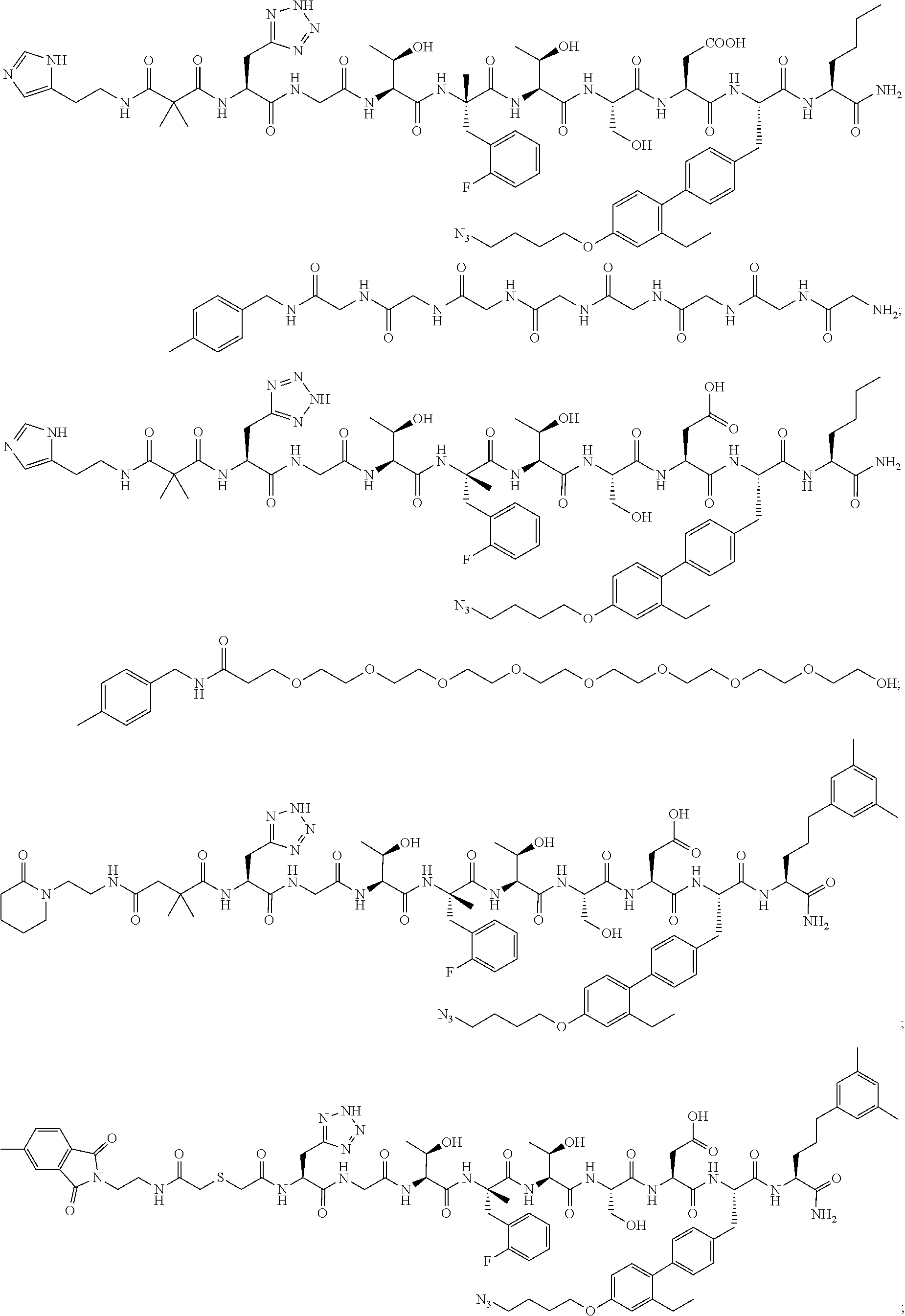 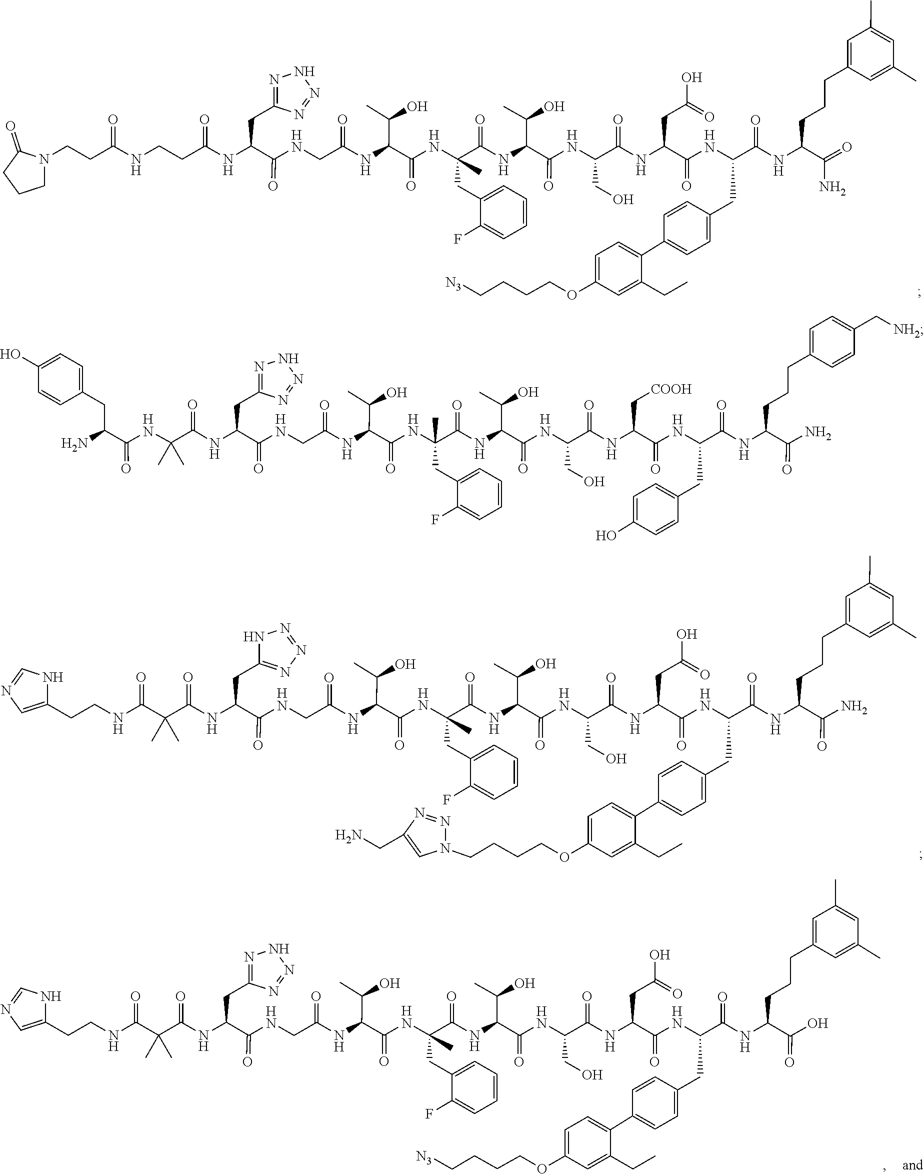 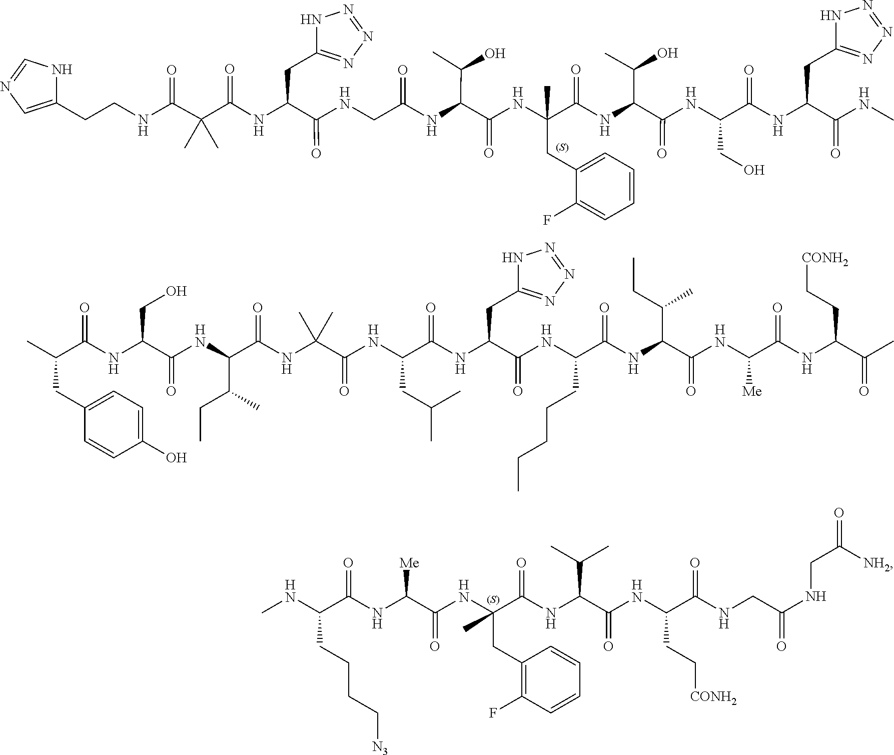
or a pharmaceutically acceptable salt thereof.

136. The antibody-tethered drug conjugate of claim 133, wherein P is a payload having the structure disclosed as SEQ ID NO: 519: 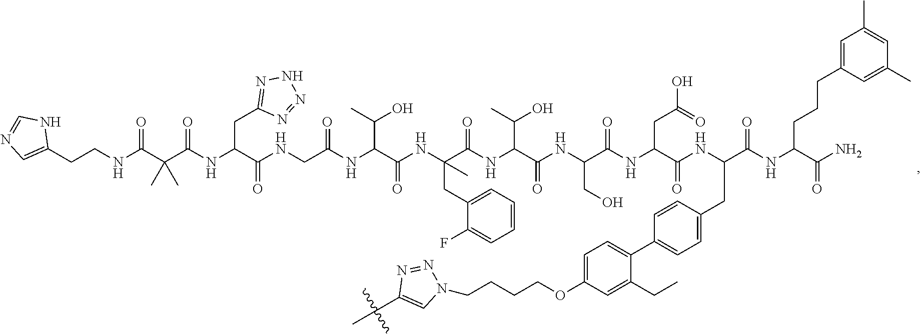 [SMILES: *c1cn(CCCCOc2ccc(-c3ccc(CC(NC(=O)C(CC(=O)O)NC(=O)C(CO)NC(=O)C(NC(=O)C(C)(Cc4ccccc4F)NC(=O)C(NC(=O)CNC(=O)C(Cc4nn[nH]n4)NC(=O)C(C)(C)C(=O)NCCc4cnc[nH]4)C(C)O)C(C)O)C(=O)NC(CCCc4cc(C)cc(C)c4)C(N)=O)cc3)c(CC)c2)nn1]
wherein 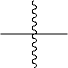
is the point of attachment of the payload to the antibody or the antigen-binding fragment thereof directly or through a linker,
or a pharmaceutically acceptable salt thereof.

137. The antibody-tethered drug conjugate of claim 136, wherein the payload has the structure disclosed as SEQ ID NO: 519:

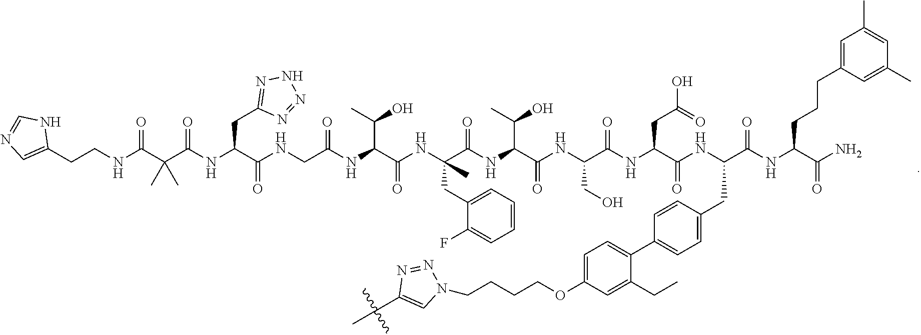 [SMILES: *c1cn(CCCCOc2ccc(-c3ccc(C[C@H](NC(=O)[C@H](CC(=O)O)NC(=O)[C@H](CO)NC(=O)[C@@H](NC(=O)[C@](C)(Cc4ccccc4F)NC(=O)[C@@H](NC(=O)CNC(=O)[C@H](Cc4nn[nH]n4)NC(=O)C(C)(C)C(=O)NCCc4cnc[nH]4)[C@@H](C)O)[C@@H](C)O)C(=O)N[C@@H](CCCc4cc(C)cc(C)c4)C(N)=O)cc3)c(CC)c2)nn1]

138. The antibody-tethered drug conjugate of claim 133, comprising a linker-payload having the structure disclosed as SEQ ID NO: 507: 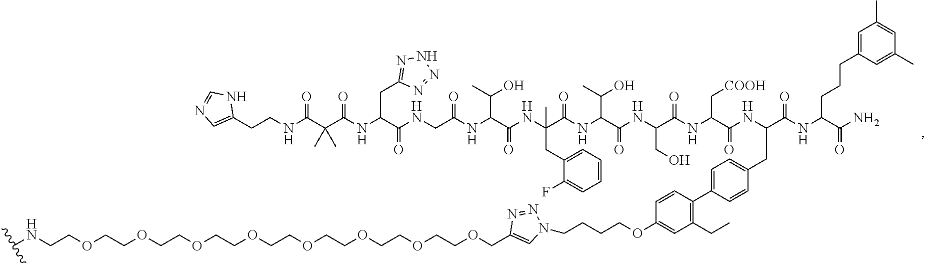 [SMILES: *NCCOCCOCCOCCOCCOCCOCCOCCOCc1cn(CCCCOc2ccc(-c3ccc(CC(NC(=O)C(CC(=O)O)NC(=O)C(CO)NC(=O)C(NC(=O)C(C)(Cc4ccccc4F)NC(=O)C(NC(=O)CNC(=O)C(Cc4nn[nH]n4)NC(=O)C(C)(C)C(=O)NCCc4cnc[nH]4)C(C)O)C(C)O)C(=O)NC(CCCc4cc(C)cc(C)c4)C(N)=O)cc3)c(CC)c2)nn1]
wherein 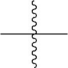
is the point of attachment of the linker-payload to a Qtag of the antibody or antigen-binding fragment thereof;
or a pharmaceutically acceptable salt thereof.

139. The antibody-tethered drug conjugate of claim 138, wherein the linker-payload has the structure disclosed as SEQ ID NO: 507:

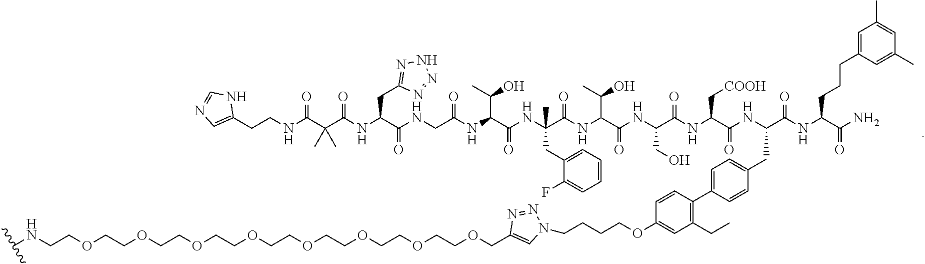

140. The antibody-tethered drug conjugate of claim 133, wherein the antibody or antigen-binding fragment thereof comprises:
(a) a heavy chain immunoglobulin that comprises the amino acid sequence set forth in SEQ ID NO: 42, and a light chain immunoglobulin that comprises the amino acid sequence set forth in SEQ ID NO: 44;
(b) a heavy chain immunoglobulin that comprises the amino acid sequence set forth in SEQ ID NO: 62, and a light chain immunoglobulin that comprises the amino acid sequence set forth in SEQ ID NO: 64;
(c) a heavy chain immunoglobulin that comprises the amino acid sequence set forth in SEQ ID NO: 82, and a light chain immunoglobulin that comprises the amino acid sequence set forth in SEQ ID NO: 84;
(d) a heavy chain immunoglobulin that comprises the amino acid sequence set forth in SEQ ID NO: 102, and a light chain immunoglobulin that comprises the amino acid sequence set forth in SEQ ID NO: 104;
(e) a heavy chain immunoglobulin that comprises the amino acid sequence set forth in SEQ ID NO: 122, and a light chain immunoglobulin that comprises the amino acid sequence set forth in SEQ ID NO: 124;
(f) a heavy chain immunoglobulin that comprises the amino acid sequence set forth in SEQ ID NO: 142, and a light chain immunoglobulin that comprises the amino acid sequence set forth in SEQ ID NO: 144;
(g) a heavy chain immunoglobulin that comprises the amino acid sequence set forth in SEQ ID NO: 162, and a light chain immunoglobulin that comprises the amino acid sequence set forth in SEQ ID NO: 164;
(h) a heavy chain immunoglobulin that comprises the amino acid sequence set forth in SEQ ID NO: 182, and a light chain immunoglobulin that comprises the amino acid sequence set forth in SEQ ID NO: 184;
(i) a heavy chain immunoglobulin that comprises the amino acid sequence set forth in SEQ ID NO: 203, and a light chain immunoglobulin that comprises the amino acid sequence set forth in SEQ ID NO: 205;
(j) a heavy chain immunoglobulin that comprises the amino acid sequence set forth in SEQ ID NO: 223, and a light chain immunoglobulin that comprises the amino acid sequence set forth in SEQ ID NO: 225;
(k) a heavy chain immunoglobulin that comprises the amino acid sequence set forth in SEQ ID NO: 243, and a light chain immunoglobulin that comprises the amino acid sequence set forth in SEQ ID NO: 245;
(l) a heavy chain immunoglobulin that comprises the amino acid sequence set forth in SEQ ID NO: 263, and a light chain immunoglobulin that comprises the amino acid sequence set forth in SEQ ID NO: 265;
(m) a heavy chain immunoglobulin that comprises the amino acid sequence set forth in SEQ ID NO: 267, and a light chain immunoglobulin that comprises the amino acid sequence set forth in SEQ ID NO: 269;
(n) a heavy chain immunoglobulin that comprises the amino acid sequence set forth in SEQ ID NO: 271, and a light chain immunoglobulin that comprises the amino acid sequence set forth in SEQ ID NO: 273;
(o) a heavy chain immunoglobulin that comprises the amino acid sequence set forth in SEQ ID NO: 291, and a light chain immunoglobulin that comprises the amino acid sequence set forth in SEQ ID NO: 293;
(p) a heavy chain immunoglobulin that comprises the amino acid sequence set forth in SEQ ID NO: 311, and a light chain immunoglobulin that comprises the amino acid sequence set forth in SEQ ID NO: 313;
(q) a heavy chain immunoglobulin that comprises the amino acid sequence set forth in SEQ ID NO: 331, and a light chain immunoglobulin that comprises the amino acid sequence set forth in SEQ ID NO: 333;
(r) a heavy chain immunoglobulin that comprises the amino acid sequence set forth in SEQ ID NO: 351, and a light chain immunoglobulin that comprises the amino acid sequence set forth in SEQ ID NO: 353;
(s) a heavy chain immunoglobulin that comprises the amino acid sequence set forth in SEQ ID NO: 371, and a light chain immunoglobulin that comprises the amino acid sequence set forth in SEQ ID NO: 373;
(t) a heavy chain immunoglobulin that comprises the amino acid sequence set forth in SEQ ID NO: 391, and a light chain immunoglobulin that comprises the amino acid sequence set forth in SEQ ID NO: 393;
(u) a heavy chain immunoglobulin that comprises the amino acid sequence set forth in SEQ ID NO: 411, and a light chain immunoglobulin that comprises the amino acid sequence set forth in SEQ ID NO: 413;
(v) a heavy chain immunoglobulin that comprises the amino acid sequence set forth in SEQ ID NO: 414, and a light chain immunoglobulin that comprises the amino acid sequence set forth in SEQ ID NO: 84; or
(w) a heavy chain immunoglobulin that comprises the amino acid sequence set forth in SEQ ID NO: 416, and a light chain immunoglobulin that comprises the amino acid sequence set forth in SEQ ID NO: 84;
optionally, wherein the heavy chain immunoglobulin does not comprise a C-terminal lysine or lysine and glycine;
wherein the structure of a linker-payload that is conjugated to amino acids 1-6 (LLQGSG (SEQ ID NO: 18)) of SEQ ID NO: 44, 64, 84, 104, 124, 144, 164, 184, 205; 225; 245; 265; 269; 273; 293; 313; 333; 353; 373; 393; or 413 via amino acid 3 (Gln) is represented by: (SEQ ID NOS 506-507, respectively, in order of appearance) 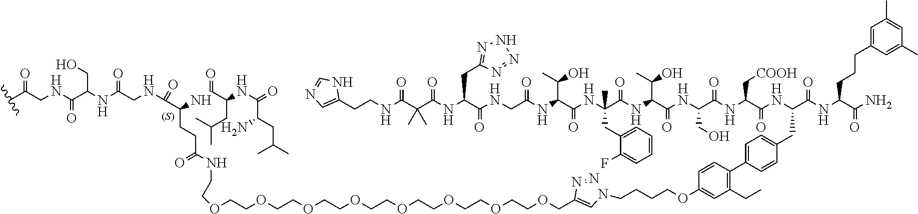
wherein 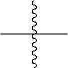
is the point of attachment of the amino acids 1-6 (LLQGSG (SEQ ID NO: 18)) to amino acid 7 of SEQ ID NO: 44, 64, 84, 104, 124, 144, 164, 184, 205; 225; 245; 265; 269; 273; 293; 313; 333; 353; 373; 393; or 413,
or a pharmaceutically acceptable salt thereof.

141. An isolated antibody or antigen-binding fragment thereof that specifically binds to Glucagon-like peptide-1 receptor (GLP1R), which
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or
a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof, optionally wherein the light chain immunoglobulin lacks the N-terminal residues LLQGSG (SEQ ID NO: 18);
(ii) is an antibody or antigen-binding fragment thereof that competes for binding to GLP1R with said antibody or antigen-binding fragment of (i); and/or
(iii) is an antibody or antigen-binding fragment thereof that binds to the same epitope of GLP1R as said antibody or antigen-binding fragment of (i);
optionally, wherein the heavy chain immunoglobulin does not comprise a C-terminal lysine or lysine and glycine; and
optionally, wherein the antibody or antigen-binding fragment is conjugated to a payload or linker-payload.

142. The antibody-tethered drug conjugate of claim 133, wherein the linker L is:
(i) attached to one or both heavy chains of the BA,
(ii) attached to one or both heavy chain variable domains of the BA,
(iii) attached to one or both light chains of the BA,
(iv) attached to one or both light chain variable domains of the BA,
(v) attached to BA via a glutamine residue, and/or
(vi) attached to BA via a lysine residue.

143. The antibody-tethered drug conjugate of claim 142, wherein the glutamine residue in (v) is:
(i) introduced to the N-terminus of one or both heavy chains of the BA,
(ii) introduced to the N-terminus of one or both light chains of the BA,
(iii) naturally present in a CH2 or CH3 domain of the BA,
(iv) introduced to the BA by modifying one or more amino acids, and/or
(v) Q295 or mutated from N297 to Q297 (N297Q).

144. The antibody-tethered drug conjugate of claim 133, wherein the antibody or antigen-binding fragment thereof is aglycosylated or deglycosylated.

145. The antibody-tethered drug conjugate of claim 133, wherein the antigen-binding fragment is an Fab fragment.

146. The antibody-tethered drug conjugate of claim 133, wherein m is 1 or 2.

147. The antibody-tethered drug conjugate of claim 133, wherein the linker L has the structure of formula (L′):
—La—Y-Lp- (L′),
wherein La is a first linker covalently attached to the BA;
Y is a group comprising a triazole, and
Lp is absent or a second linker covalently attached to the P, wherein when Lp is absent, Y is also absent.

148. The antibody-tethered drug conjugate of claim 147, wherein Y-Lp is absent or has a structure selected from the group consisting of: 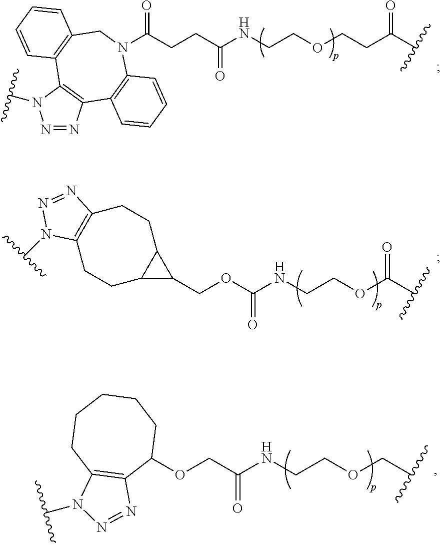
or a triazole regioisomer thereof,
wherein p is an integer from 1 to 36.

149. The antibody-tethered drug conjugate of claim 148, wherein Y has a structure selected from the group consisting of: 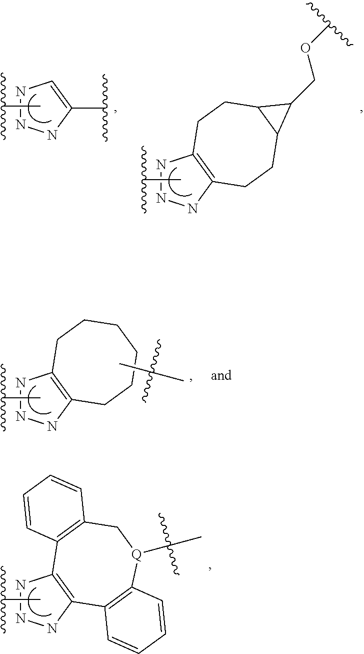
wherein Q is C or N.

150. The antibody-tethered drug conjugate of claim 147, wherein Lp comprises a polyethylene glycol (PEG) segment having 1 to 36 —CH₂CH₂O— (EG) units and/or where Lp comprises one or more amino acids selected from glycine, serine, glutamic acid, alanine, valine, and proline and combinations thereof.

151. The antibody-tethered drug conjugate of claim 147, wherein the PEG segment comprises 4 EG units, or 8 EG units, or 12 EG units, or 24 EG units.

152. The antibody-tethered drug conjugate of claim 150, wherein the Lp comprises 1 to 10 glycines and/or 1 to 6 serines.

153. The antibody-tethered drug conjugate of claim 152, wherein the Lp is selected from the group consisting of Gly-Gly-Gly-Gly-Ser (G₄S) (SEQ ID NO: 1), Ser-Gly-Gly-Gly-Gly (SG₄) (SEQ ID NO: 2), and Gly-Gly-Gly-Gly-Ser-Gly-Gly-Gly-Gly-Ser (G₄S-G₄S) (SEQ ID NO: 3).

154. The antibody-tethered drug conjugate of claim 147, wherein Lp has a structure selected from the group consisting of (SEQ ID NOS 567-568, respectively, in order of appearance): 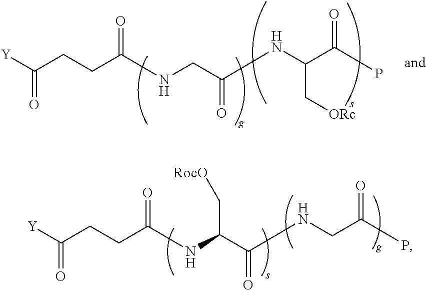
wherein Y is the group comprising a triazole and P is the payload, and wherein Rc is selected from H and glucose, g is an integer from 1 to 10 and s is an integer from 0 to 4.

155. The antibody-tethered drug conjugate of claim 147, wherein Y-Lp has a structure selected from the group consisting of (SEQ ID NOS 453-458, respectively, in order of appearance): 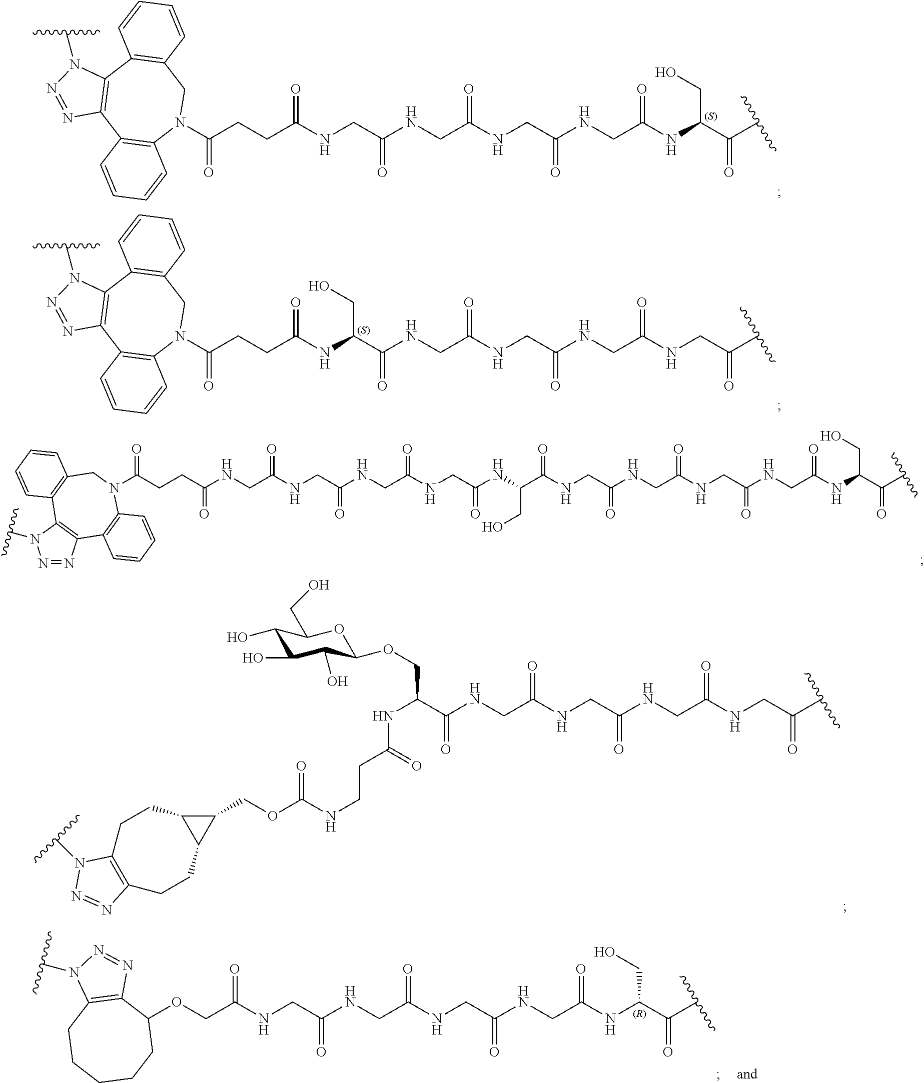 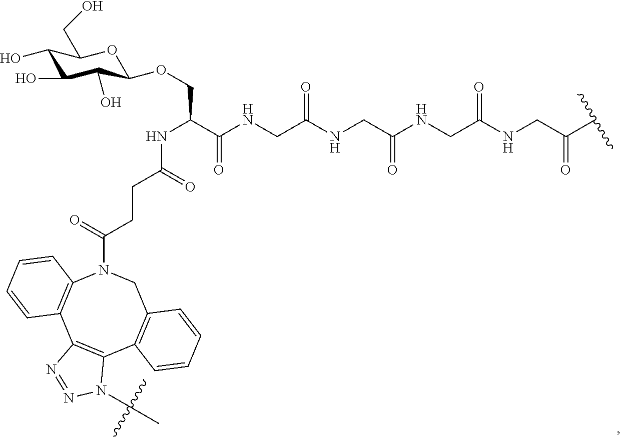
or a triazole regioisomer thereof.

156. The antibody-tethered drug conjugate of claim 147, wherein La comprises a polyethylene glycol (PEG) segment having 1 to 36 —CH₂CH₂O— (EG) units, and/or wherein La comprises one or more amino acids selected from glycine, threonine, serine, glutamine, glutamic acid, alanine, valine, leucine, and proline and combinations thereof, and/or wherein La comprises a —(CH₂)₂₋₂₄— chain.

157. The antibody-tethered drug conjugate of claim 156, wherein the PEG segment comprises 4 EG units, or 8 EG units, or 12 EG units, or 24 EG units.

158. The antibody-tethered drug conjugate of claim 147, wherein La has a structure selected from the group consisting of:

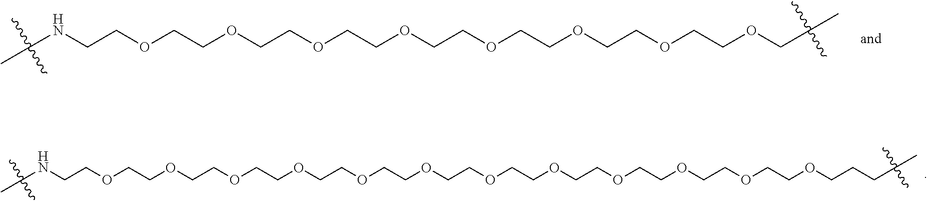 [SMILES: *COCCOCCOCCOCCOCCOCCOCCOCCN* ; *CCCOCCOCCOCCOCCOCCOCCOCCOCCOCCOCCOCCOCCN*]

159. The antibody-tethered drug conjugate of claim 156, wherein the La comprises 1 to 10 glycines and 1 to 6 serines.

160. The antibody-tethered drug conjugate of claim 159, wherein the La is selected from the group consisting of Gly-Gly-Gly-Gly-Ser (G₄S) (SEQ ID NO: 1), Ser-Gly-Gly-Gly-Gly (SG₄) (SEQ ID NO: 2), Gly-Gly-Ser-Gly-Gly-Ser-Gly-Gly (G₂S-G₂S-G₂) (SEQ ID NO: 438), and Gly-Gly-Gly-Gly-Ser-Gly-Gly-Gly-Gly (G₄S-G₄) (SEQ ID NO: 419).

161. The antibody-tethered drug conjugate of claim 156, wherein the La is selected from the group consisting of SE ID NOS 459-462 respectively in order of appearance):

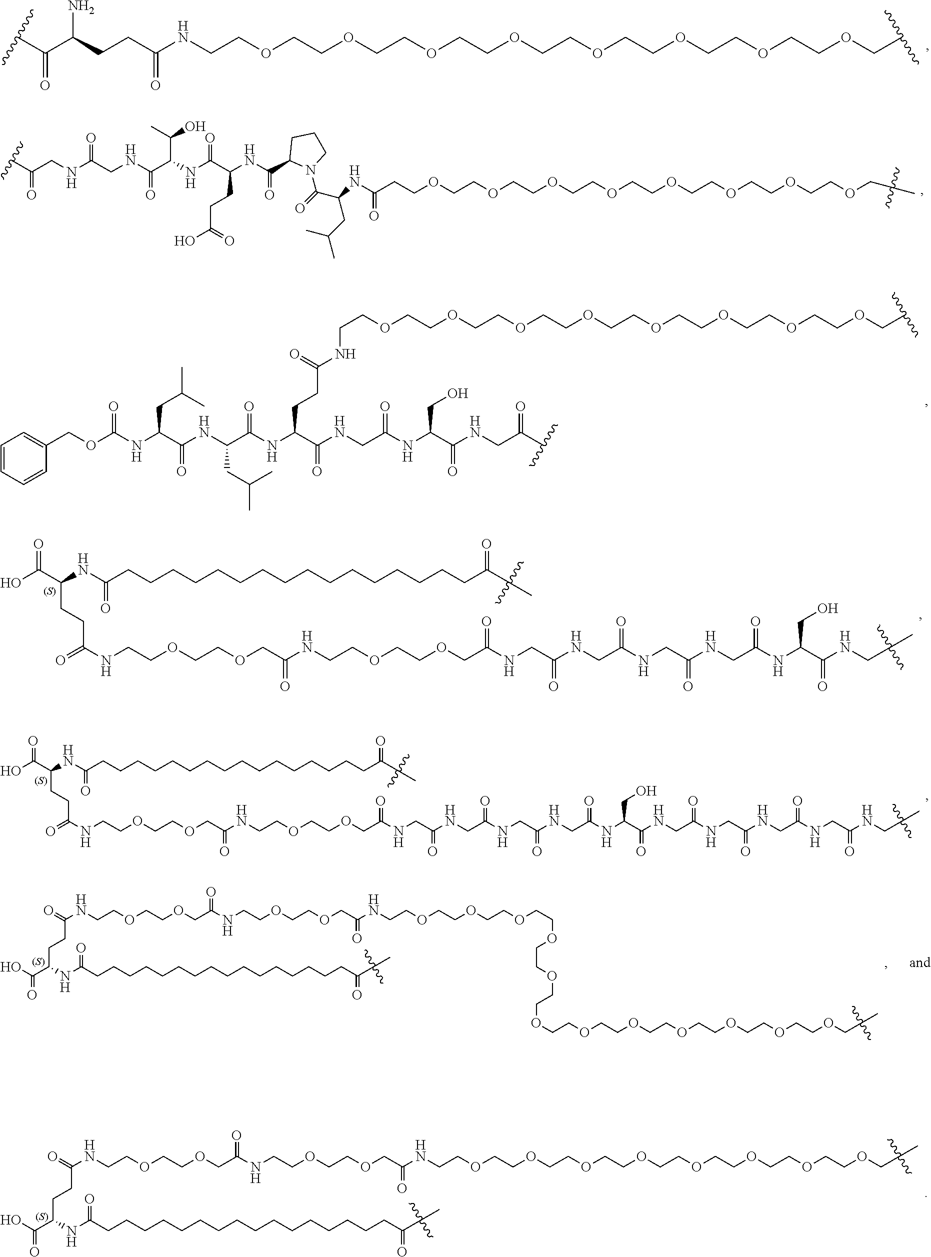

162. The antibody-tethered drug conjugate of claim 133, wherein P has the structure disclosed as SEQ ID NO: 463:

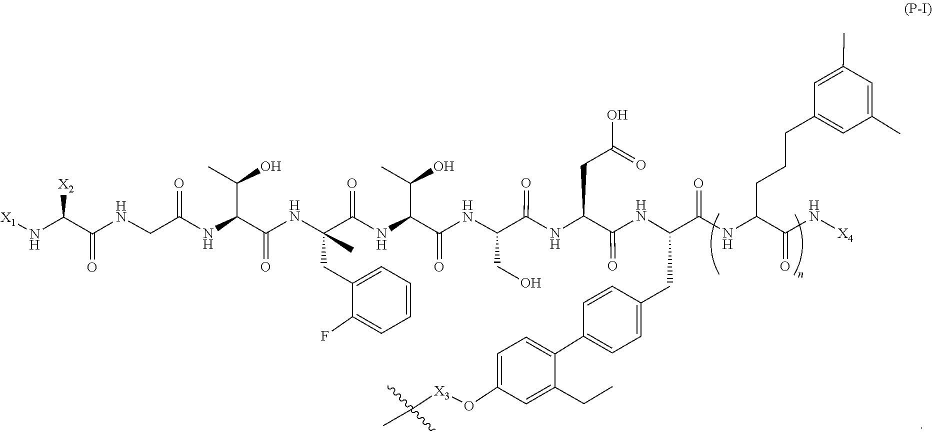

163. The antibody-tethered drug conjugate of claim 134, wherein
(i) X₁ is H; X₂ is 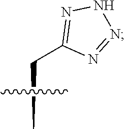
X₃ is selected from —(CH₂)₂₋₆—NH—and —(CH₂)₂₋₆-Tr-, where Tr is a triazole moiety; n is 1, and X₄ₐ is H,
(ii) X₁ is 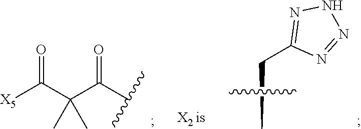
X₃ is —(CH₂)₂₋₆—NH—; n is 1; X₄ₐ is H, and X₅ is selected from —OH, —NH₂, —NH—OH, and 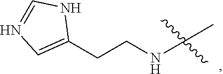 [SMILES: *NCCc1cnc[nH]1]
(iii) X₁ is H 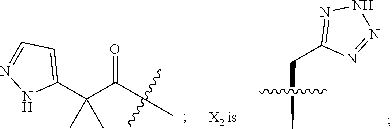
X₃ is —(CH₂)₂₋₆—NH—; n is 1, and X₄ₐ is H,
(iv) X₁ is 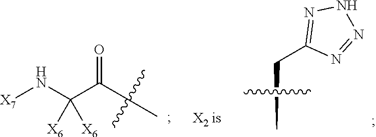
X₃ is —(CH₂)₂₋₆—NH—; n is 1; X₄ₐ is H; X₆ is independently at each occurrence selected from H and —CH₂OH, and X₇ is H,
(v) X₁ is 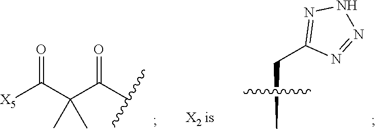
X₃ is —(CH₂)₂₋₆-Tr-, where Tr is a triazole moiety; n is 1; X₄ₐ is H, and X₅ is 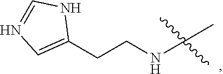 [SMILES: *NCCc1cnc[nH]1]
(vi) X₁ is 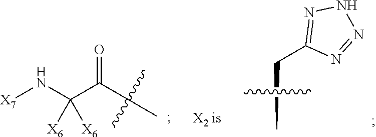
X₃ is —(CH₂)₂₋₆—NH—; n is 1; X₄ₐ is H; X₆ is independently at each occurrence selected from H and —CH₃; X₇ is 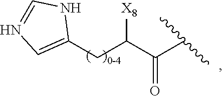
and X₈ is —NH₂,
(vii) X₁ is 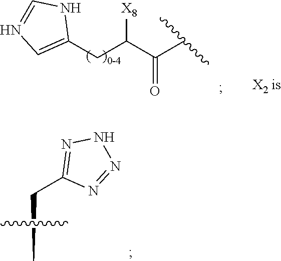
X₃ is —(CH₂)₂₋₆—NH—; n is 1; X₄ₐ is H, and X₈ is H,
(viii) X₁ is 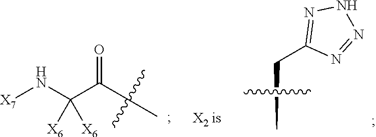
X₃ is —(CH₂)₂₋₆—NH—; n is 1; X₄ₐ is H; X₆ is H at each occurrence; X₇ is 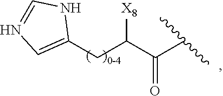
and X₈ is H,
(ix) X₁ is 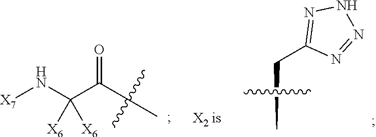
X₃ is —(CH₂)₂₋₆—NH—; n is 1; X₄ₐ is H; X₆ is independently at each occurrence selected from H and —CH₃; X₇ is 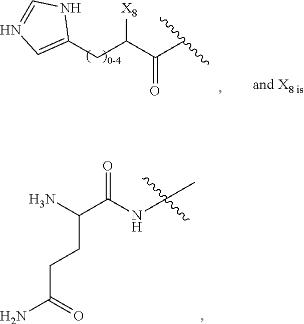
(x) X₁ is 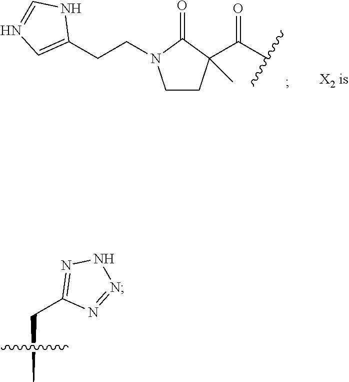
X₃ is —(CH₂)₂₋₆—NH—; n is 1, and X₄ₐ is H,
(xi) X₁ is 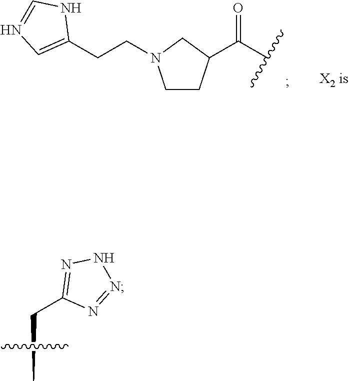
X₃ is —(CH₂)₂₋₆—NH—; n is 1, and X₄ₐ is H,
(xii) X₁ is 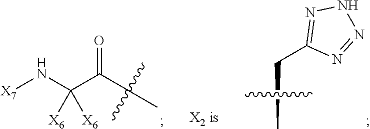
X₃ is —(CH₂)₂₋₆—NH—; n is 1; X₄ₐ is H; X₆ is independently at each occurrence selected from H and —CH₃, and X₇ is 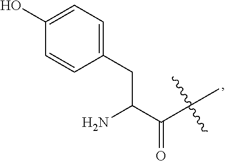 [SMILES: *C(=O)C(N)Cc1ccc(O)cc1]
(xiii) X₁ is 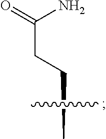
X₃ is —(CH₂)₂₋₆—NH—; n is 1, and X₄ₐ is H,
(xiv) X₁ is 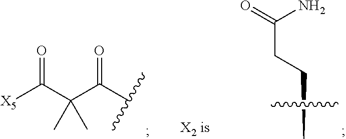
X₃ is —(CH₂)₂₋₆—NH—; n is 1; X₄ₐ is H, and X₅ is 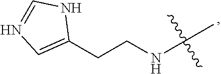 [SMILES: *NCCc1cnc[nH]1]
(xv) X₁ is 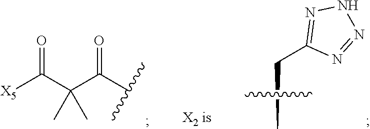
X₃ is —(CH₂)₂₋₆—NH—; n is 0; X₄ₐ is phenyl, and X₅ is 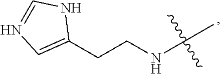 [SMILES: *NCCc1cnc[nH]1]
(xvi) X₁ is 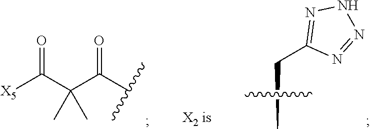
X₃ is —(CH₂)₂₋₆—NH—; n is 1; X₄ₐ is phenyl, and X₅ is 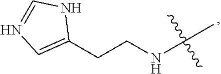 [SMILES: *NCCc1cnc[nH]1]
or
(xvii) X₁ is 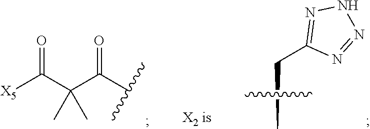
X₃ is —(CH₂)₂₋₆—NH—; X₄ₐ is H, and X₅ is

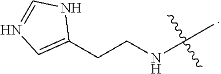 [SMILES: *NCCc1cnc[nH]1]

164. The antibody-tethered drug conjugate of claim 133, wherein P has the structure selected from the group consisting of (SEQ ID NOS 465, 576, 466-495, 610, 496-497, 611, 498-505, respectively, in order of appearance):

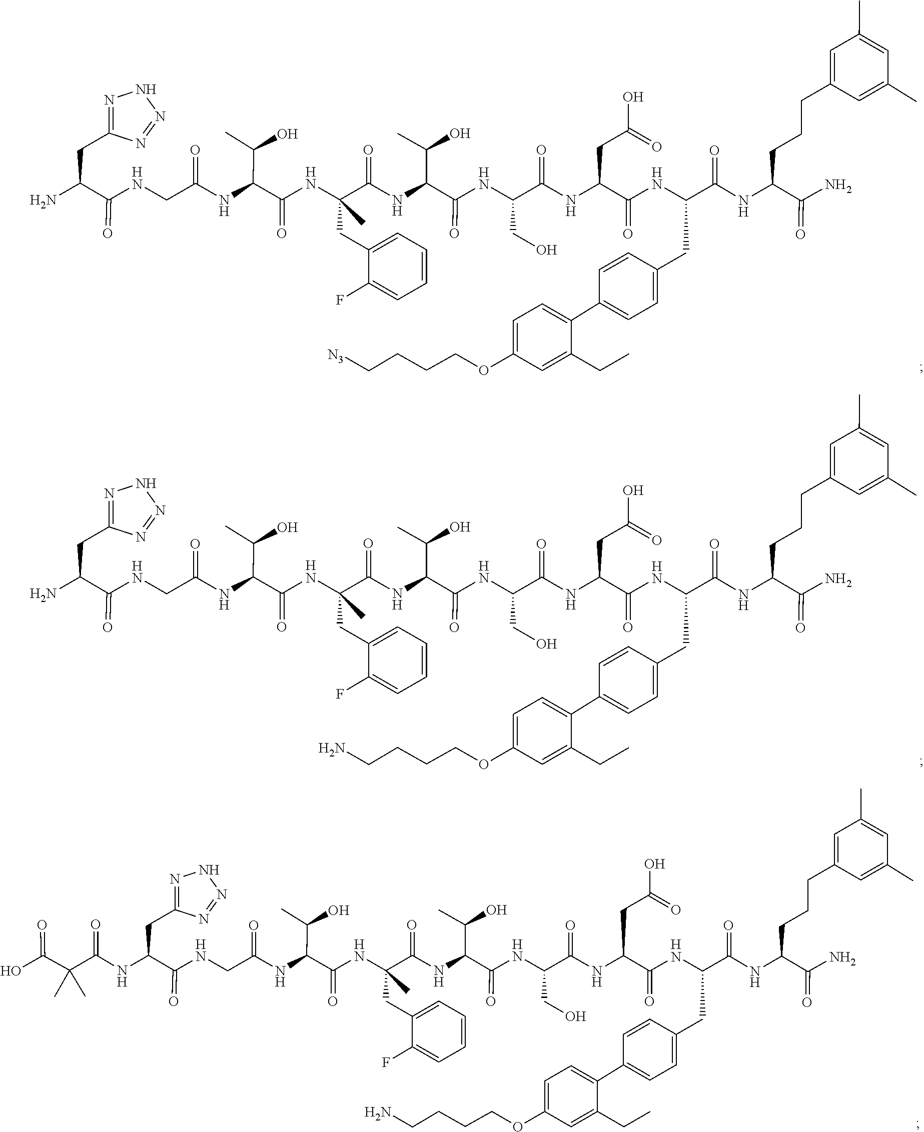

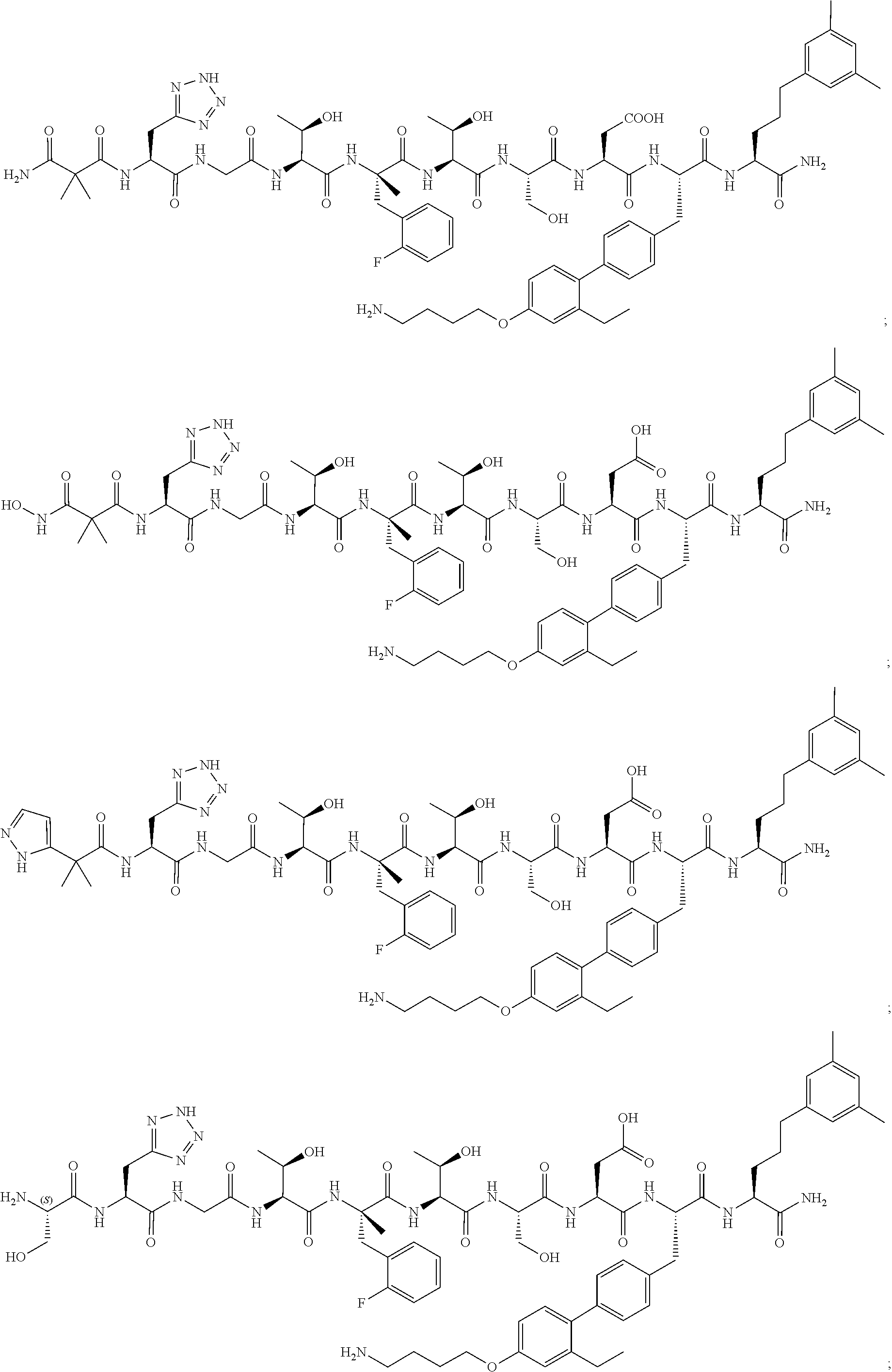

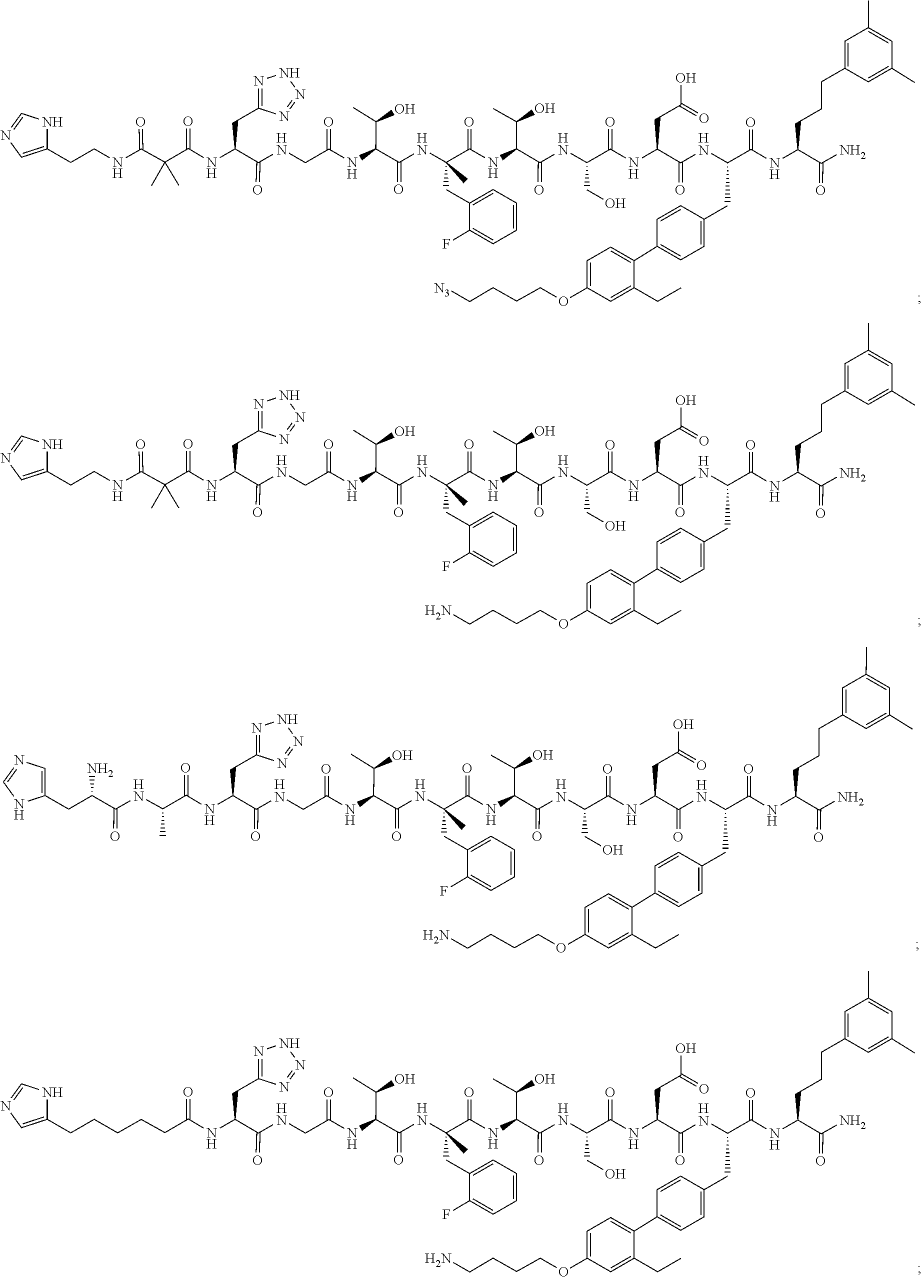

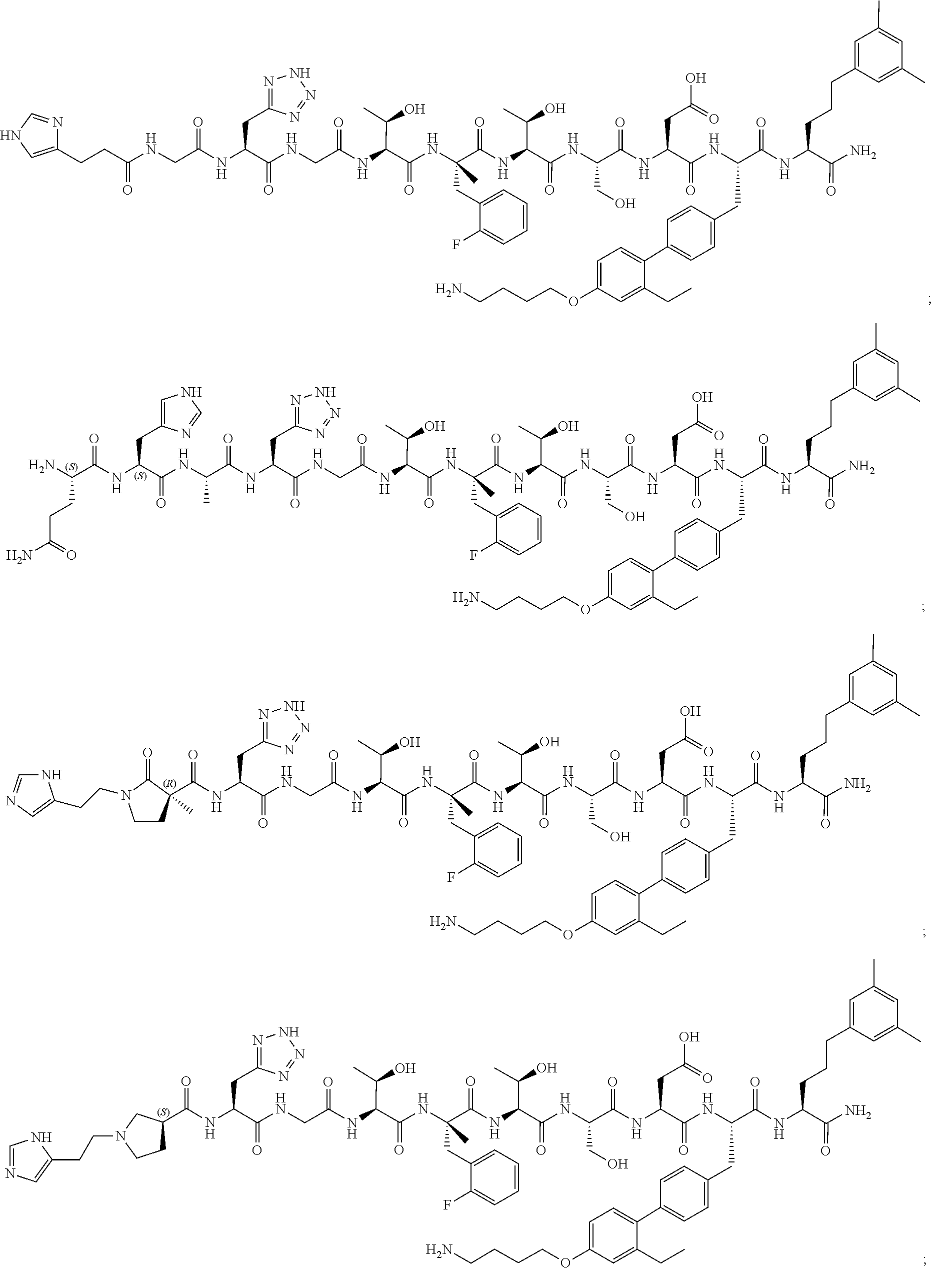

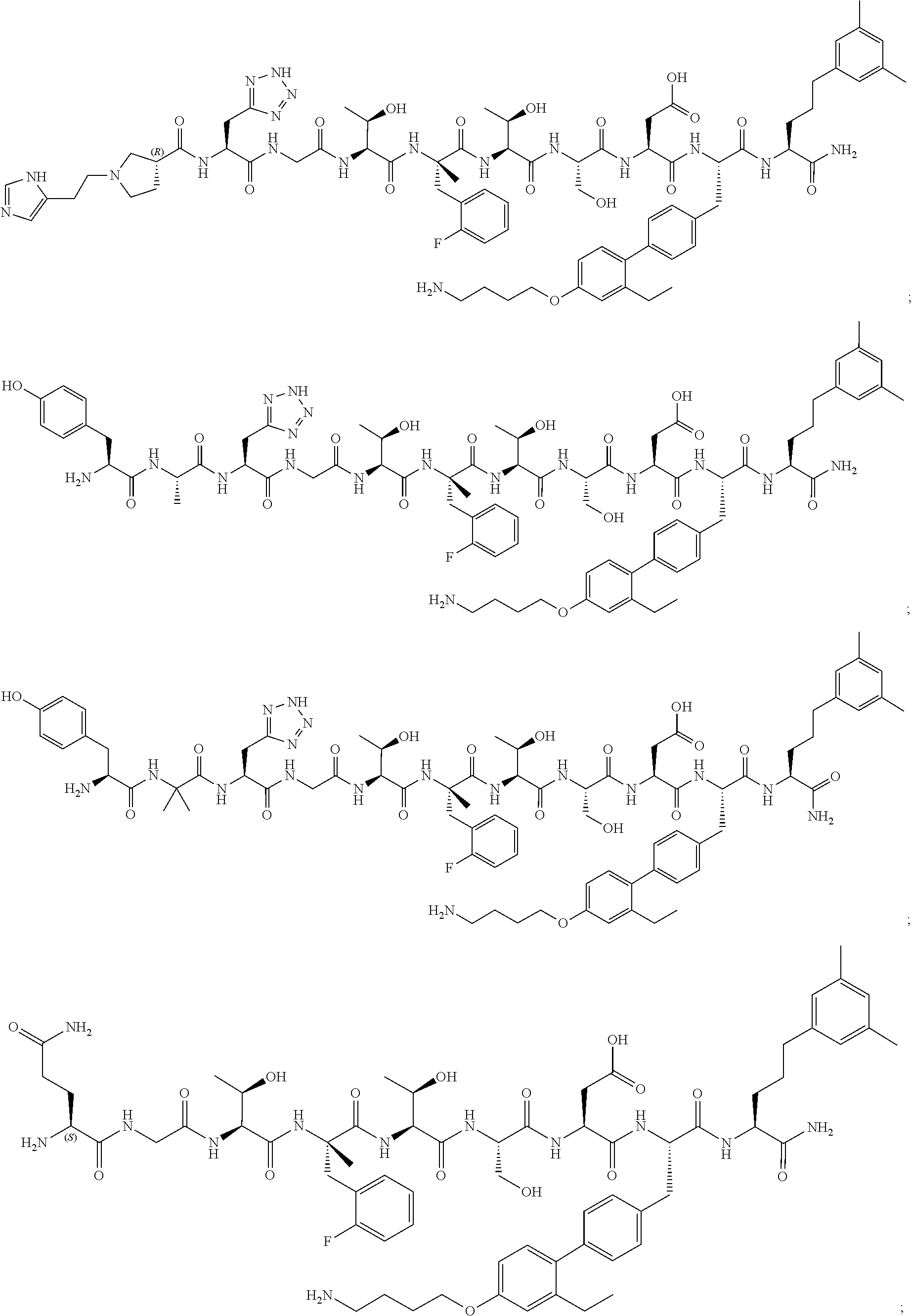

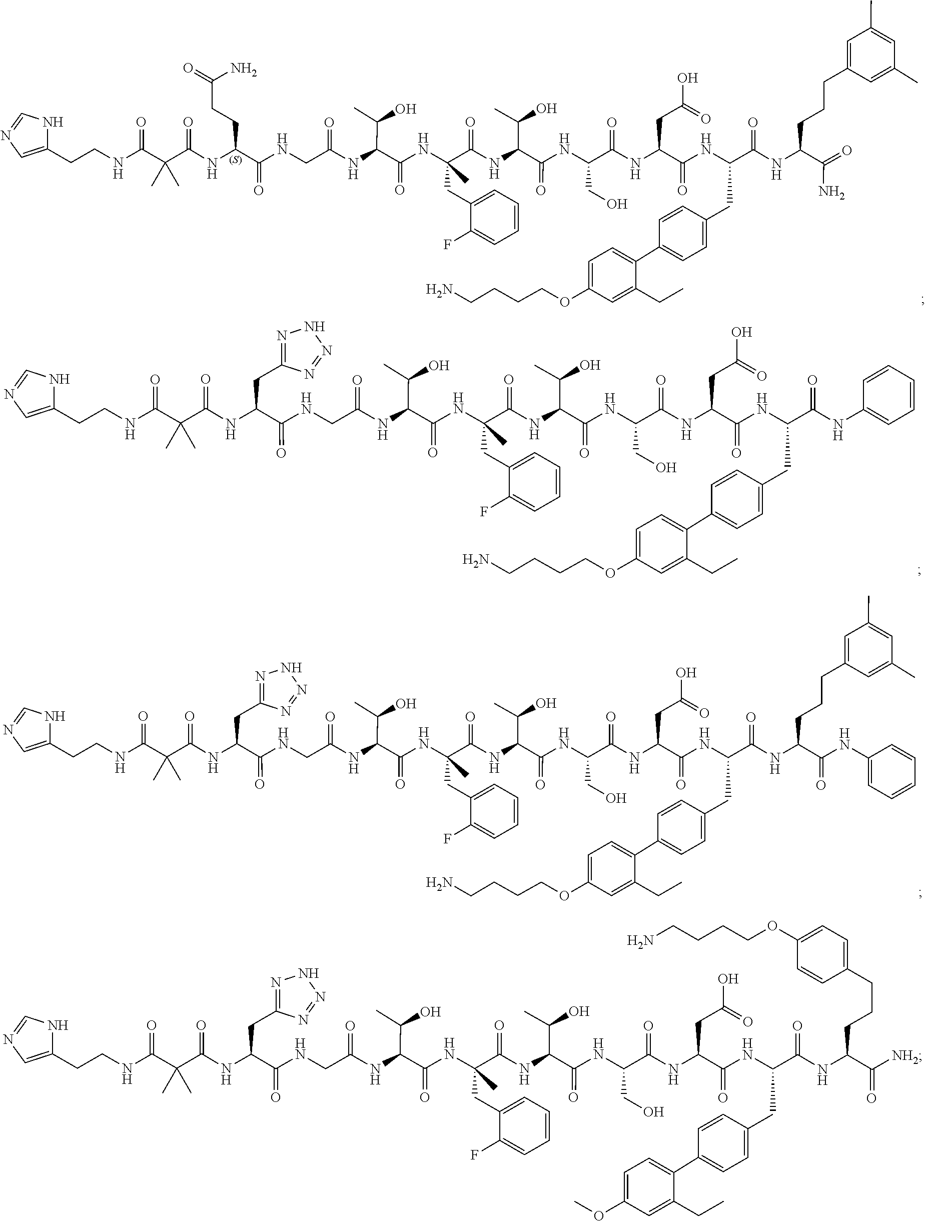

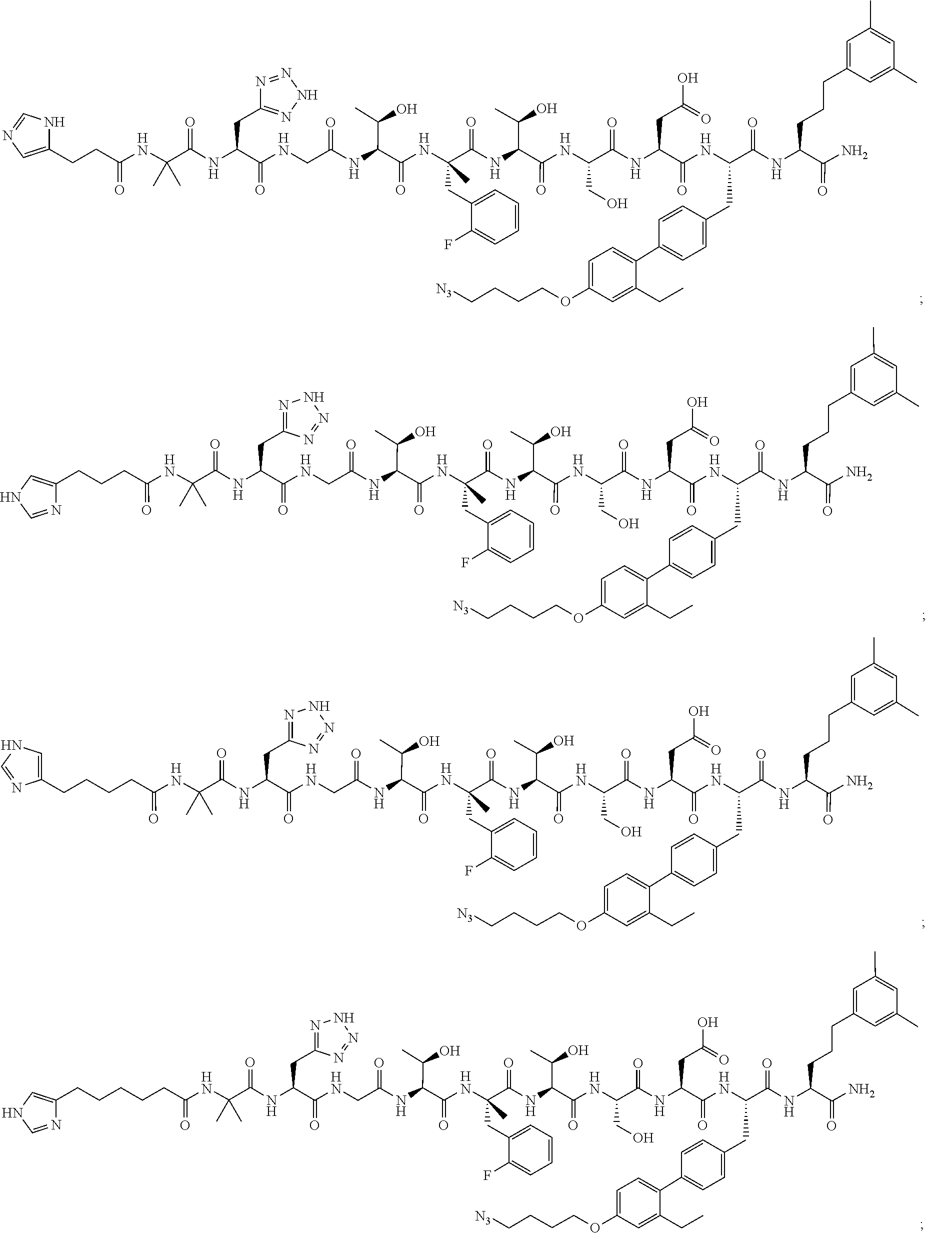

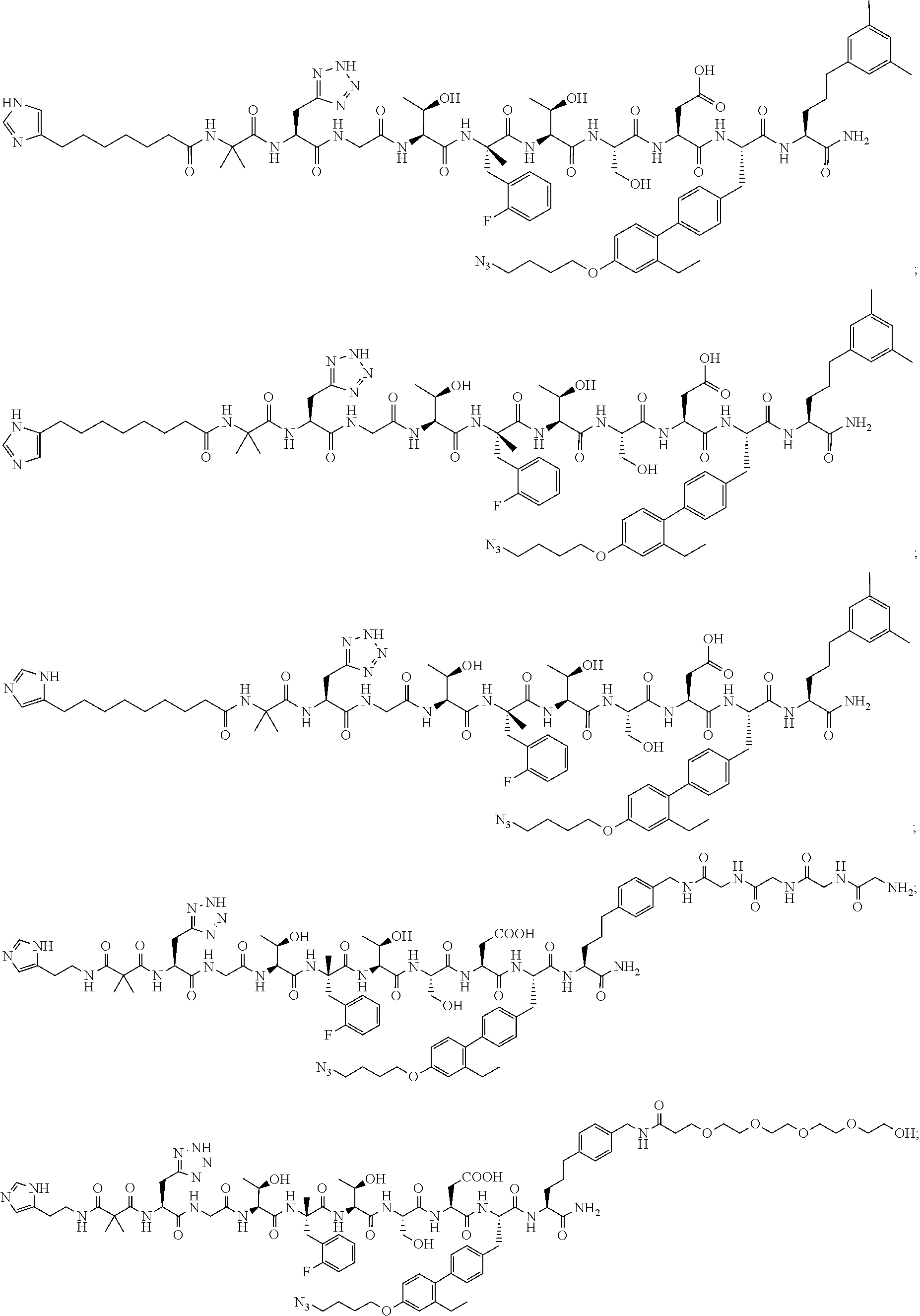

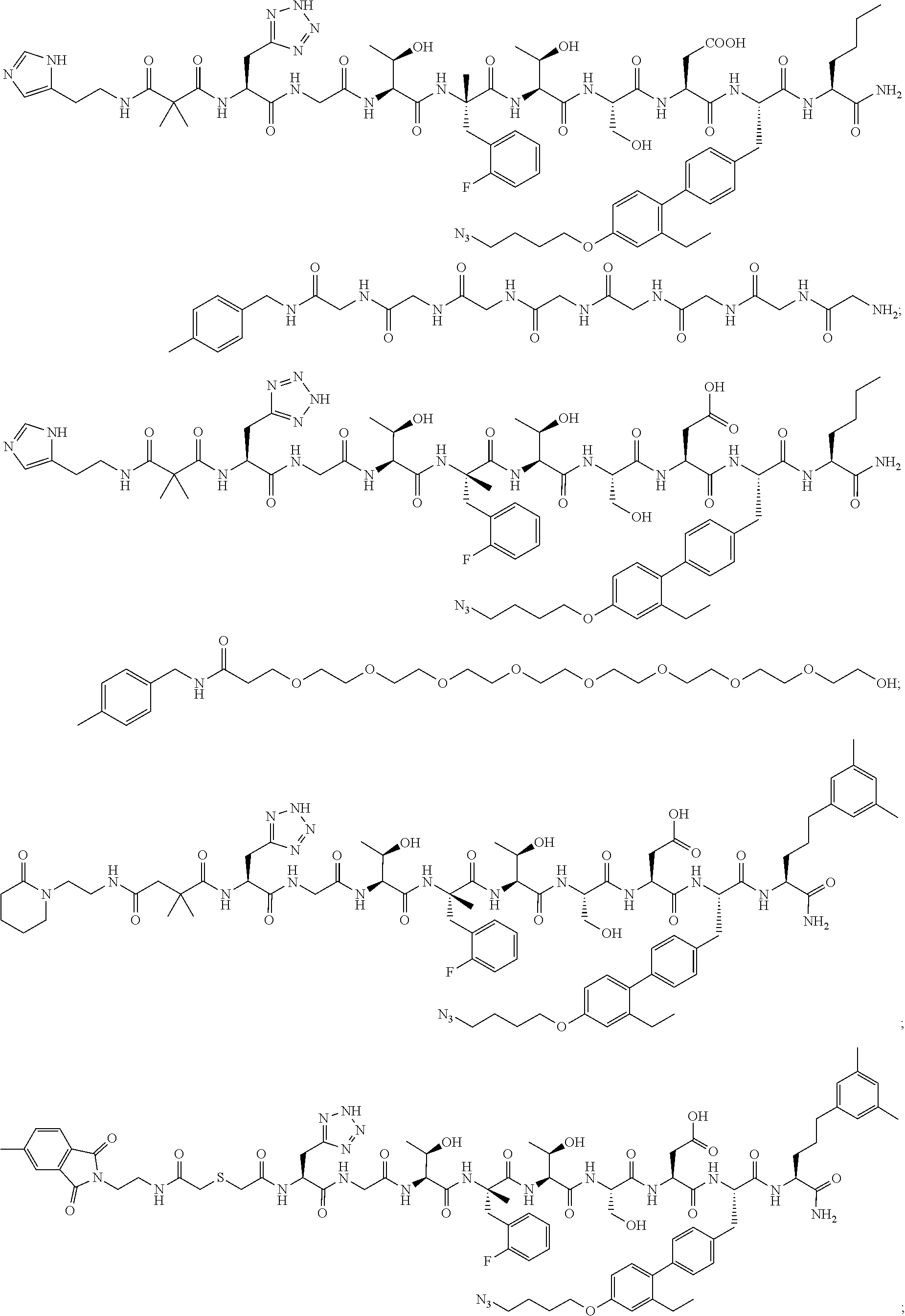

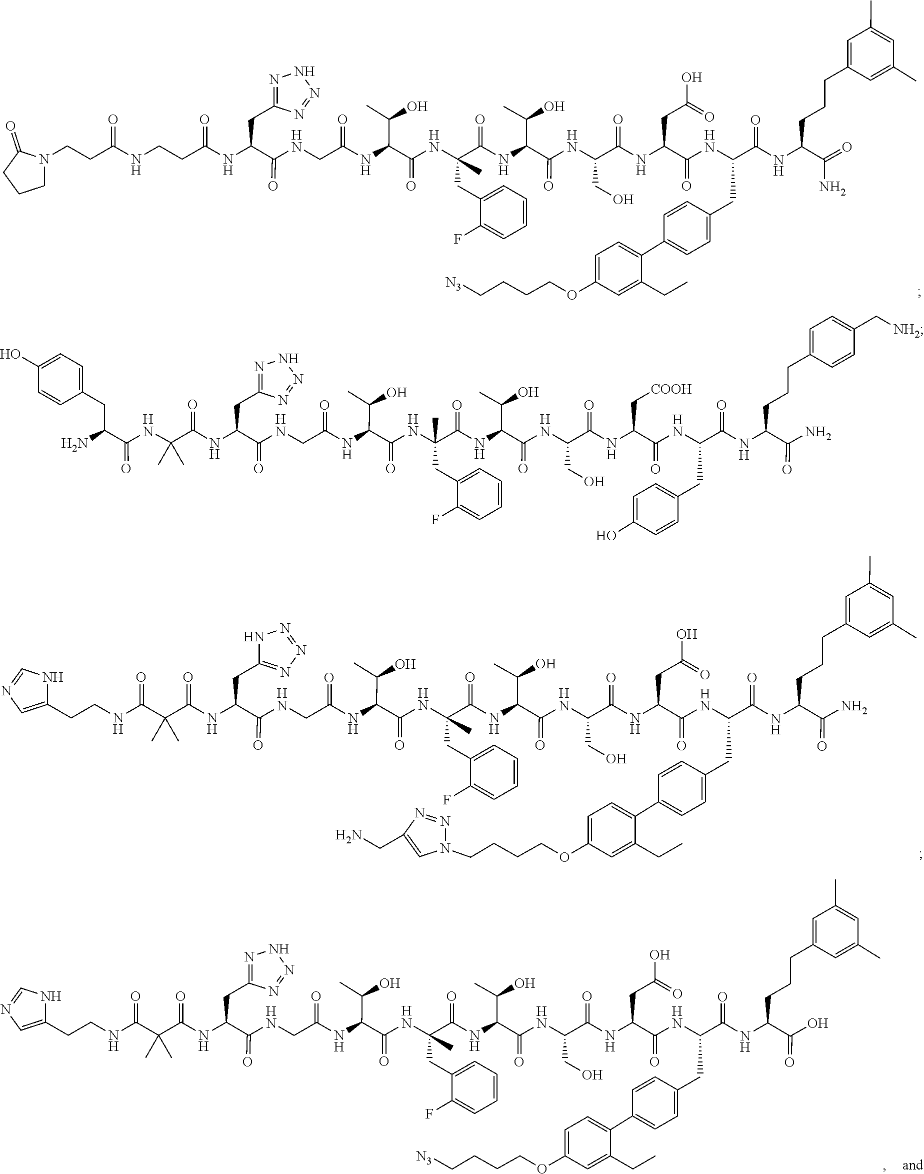

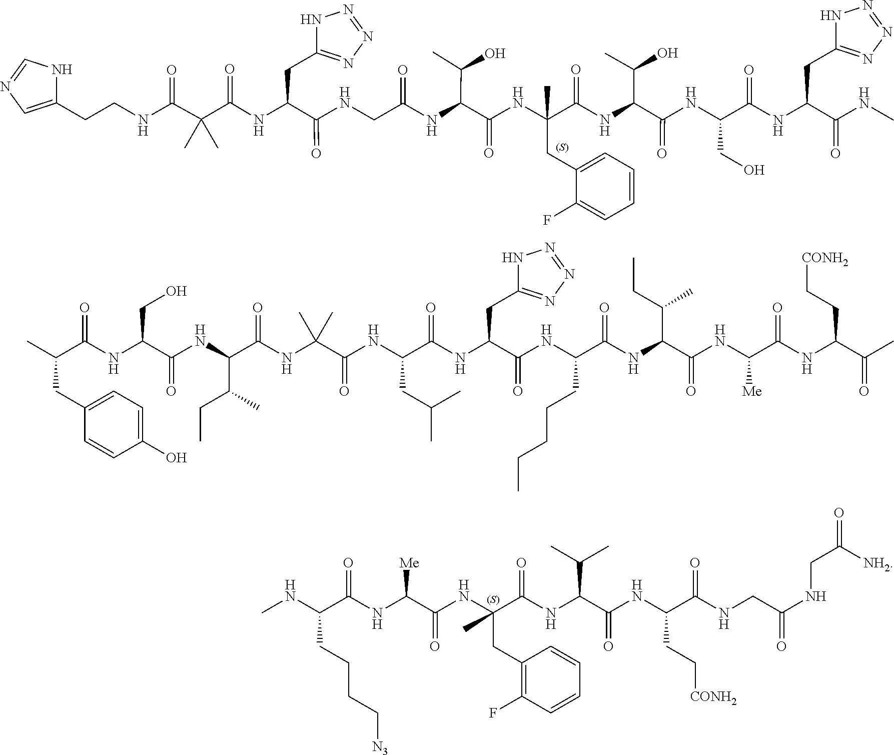

165. The antibody-tethered drug conjugate of claim 133, wherein the antibody-tethered drug conjugate has a half life of longer than 7 days in plasma, and/or wherein the antibody-tethered drug conjugate does not bind to G protein-coupled receptors (GPCRs) other than GLP1R.

166. The antibody-tethered drug conjugate of claim 133, comprising a payload that is conjugated to a linker which is conjugated to one or both of two immunoglobulin heavy chains or variable regions thereof and/or one or both of two immunoglobulin light chains or variable regions thereof of the antibody or antigen-binding fragment thereof, and having the structure disclosed as SEQ ID NO: 447: 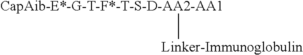
wherein
immunoglobulin is the immunoglobulin chain of the antibody or antigen-binding fragment;
CapAib is 3-((2-(1H-imidazol-5-yl)ethyl)amino)-2,2-dimethyl-3-oxopropanoic acid;
E* is (S)-2-amino-3-(2H-tetrazol-5-yl)propanoic acid;
G is glycine;
T is threonine;
F* is (S)-2-amino-3-(2-fluorophenyl)-2-methylpropanoic acid;
S is serine;
D is aspartate;
AA2 is (S)-2-amino-3-(4′-(4-(4-(25-amino-2,5,8,11,14,17,20,23-octaoxapentacosyl)-1H-1,2,3-triazol-1-yl)butoxy)-2′-ethyl-[1,1′-biphenyl]-4-yl)propanoic acid [AA2 includes linker]; and
AA1=(S)-2-amino-5-(3,5-dimethylphenyl)pentanamide, or a pharmaceutically acceptable salt thereof.

167. The antibody-tethered drug conjugate of claim 133, wherein the linker is conjugated to the immunoglobulin heavy chain and/or light chain or variable region thereof by a Qtag, optionally via a glutamine (Q) residue of the Qtag.

168. The antibody-tethered drug conjugate of claim 167, wherein the Qtag comprises the amino acid sequence LLQGG (SEQ ID NO: 6), LLQG (SEQ ID NO: 7), LSLSQG (SEQ ID NO:
8), GGGLLQGG (SEQ ID NO: 9), GLLQG (SEQ ID NO: 10), LLQ, GSPLAQSHGG (SEQ ID NO: 11), GLLQGGG (SEQ ID NO: 12), GLLQGG (SEQ ID NO: 13), GLLQ (SEQ ID NO: 14), LLQLLQGA (SEQ ID NO: 15), LLQGA (SEQ ID NO: 16), LLQYQGA (SEQ ID NO: 17), LLQGSG (SEQ ID NO: 18), LLQYQG (SEQ ID NO: 19), LLQLLQG (SEQ ID NO: 20), SLLQG (SEQ ID NO: 21), LLQLQ (SEQ ID NO: 22), LLQLLQ (SEQ ID NO: 23), LLQGSGSG (SEQ ID NO: 185) and/or LLQGR (SEQ ID NO: 24).

169. The antibody-tethered drug conjugate of claim 133, wherein:
(a) the heavy chain immunoglobulin or variable region thereof comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411, or an amino acid sequence having at least 90% sequence identity to the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; and/or
(b) the light chain immunoglobulin or variable region thereof comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413, or an amino acid sequence having at least 90% sequence identity to the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413.

170. The antibody-tethered drug conjugate of claim 133, wherein:
the heavy chain immunoglobulin or variable region thereof comprises:
(i) a CDR-H1 comprising the amino acid sequence set forth in SEQ ID NO: 28, or a variant thereof, a CDR-H2 comprising the amino acid sequence set forth in SEQ ID NO: 30, or a variant thereof, a CDR-H3 comprising the amino acid sequence set forth in SEQ ID NO: 32, or a variant thereof;
(ii) a CDR-H1 comprising the amino acid sequence set forth in SEQ ID NO: 48, or a variant thereof, a CDR-H2 comprising the amino acid sequence set forth in SEQ ID NO: 50, or a variant thereof, a CDR-H3 comprising the amino acid sequence set forth in SEQ ID NO: 52, or a variant thereof;
(iii) a CDR-H1 comprising the amino acid sequence set forth in SEQ ID NO: 68, or a variant thereof, a CDR-H2 comprising the amino acid sequence set forth in SEQ ID NO: 70, or a variant thereof, a CDR-H3 comprising the amino acid sequence set forth in SEQ ID NO: 72, or a variant thereof;
(iv) a CDR-H1 comprising the amino acid sequence set forth in SEQ ID NO: 88, or a variant thereof, a CDR-H2 comprising the amino acid sequence set forth in SEQ ID NO: 90, or a variant thereof, a CDR-H3 comprising the amino acid sequence set forth in SEQ ID NO: 92, or a variant thereof;
(v) a CDR-H1 comprising the amino acid sequence set forth in SEQ ID NO: 108, or a variant thereof, a CDR-H2 comprising the amino acid sequence set forth in SEQ ID NO: 110, or a variant thereof, a CDR-H3 comprising the amino acid sequence set forth in SEQ ID NO: 112, or a variant thereof,
(vi) a CDR-H1 comprising the amino acid sequence set forth in SEQ ID NO: 128, or a variant thereof, a CDR-H2 comprising the amino acid sequence set forth in SEQ ID NO: 130, or a variant thereof, a CDR-H3 comprising the amino acid sequence set forth in SEQ ID NO: 132, or a variant thereof,
(vii) a CDR-H1 comprising the amino acid sequence set forth in SEQ ID NO: 148, or a variant thereof, a CDR-H2 comprising the amino acid sequence set forth in SEQ ID NO: 150, or a variant thereof, a CDR-H3 comprising the amino acid sequence set forth in SEQ ID NO: 152, or a variant thereof,
(viii) a CDR-H1 comprising the amino acid sequence set forth in SEQ ID NO: 168, or a variant thereof, a CDR-H2 comprising the amino acid sequence set forth in SEQ ID NO: 170, or a variant thereof, a CDR-H3 comprising the amino acid sequence set forth in SEQ ID NO: 172, or a variant thereof,
(ix) a CDR-H1 comprising the amino acid sequence set forth in SEQ ID NO: 189, or a variant thereof, a CDR-H2 comprising the amino acid sequence set forth in SEQ ID NO: 191, or a variant thereof, a CDR-H3 comprising the amino acid sequence set forth in SEQ ID NO: 193, or a variant thereof,
(x) a CDR-H1 comprising the amino acid sequence set forth in SEQ ID NO: 209, or a variant thereof, a CDR-H2 comprising the amino acid sequence set forth in SEQ ID NO: 211, or a variant thereof, a CDR-H3 comprising the amino acid sequence set forth in SEQ ID NO: 213, or a variant thereof,
(xi) a CDR-H1 comprising the amino acid sequence set forth in SEQ ID NO: 229, or a variant thereof, a CDR-H2 comprising the amino acid sequence set forth in SEQ ID NO: 231, or a variant thereof, a CDR-H3 comprising the amino acid sequence set forth in SEQ ID NO: 233, or a variant thereof,
(xii) a CDR-H1 comprising the amino acid sequence set forth in SEQ ID NO: 249, or a variant thereof, a CDR-H2 comprising the amino acid sequence set forth in SEQ ID NO: 251, or a variant thereof, a CDR-H3 comprising the amino acid sequence set forth in SEQ ID NO: 253, or a variant thereof,
(xiii) a CDR-H1 comprising the amino acid sequence set forth in SEQ ID NO: 277, or a variant thereof, a CDR-H2 comprising the amino acid sequence set forth in SEQ ID NO: 279, or a variant thereof, a CDR-H3 comprising the amino acid sequence set forth in SEQ ID NO: 281, or a variant thereof,
(xiv) a CDR-H1 comprising the amino acid sequence set forth in SEQ ID NO: 297, or a variant thereof, a CDR-H2 comprising the amino acid sequence set forth in SEQ ID NO: 299, or a variant thereof, a CDR-H3 comprising the amino acid sequence set forth in SEQ ID NO: 301, or a variant thereof,
(xv) a CDR-H1 comprising the amino acid sequence set forth in SEQ ID NO: 317, or a variant thereof, a CDR-H2 comprising the amino acid sequence set forth in SEQ ID NO: 319, or a variant thereof a CDR-H3 comprising the amino acid sequence set forth in SEQ ID NO: 321, or a variant thereof,
(xvi) a CDR-H1 comprising the amino acid sequence set forth in SEQ ID NO: 337, or a variant thereof, a CDR-H2 comprising the amino acid sequence set forth in SEQ ID NO: 339, or a variant thereof, a CDR-H3 comprising the amino acid sequence set forth in SEQ ID NO: 341, or a variant thereof,
(xvii) a CDR-H1 comprising the amino acid sequence set forth in SEQ ID NO: 357, or a variant thereof, a CDR-H2 comprising the amino acid sequence set forth in SEQ ID NO: 359, or a variant thereof, a CDR-H3 comprising the amino acid sequence set forth in SEQ ID NO: 361, or a variant thereof, and/or
(xviii) a CDR-H1 comprising the amino acid sequence set forth in SEQ ID NO: 377, or a variant thereof, a CDR-H2 comprising the amino acid sequence set forth in SEQ ID NO: 379, or a variant thereof, a CDR-H3 comprising the amino acid sequence set forth in SEQ ID NO: 381, or a variant thereof,
(xix) a CDR-H1 comprising the amino acid sequence set forth in SEQ ID NO: 397, or a variant thereof, a CDR-H2 comprising the amino acid sequence set forth in SEQ ID NO: 399, or a variant thereof, a CDR-H3 comprising the amino acid sequence set forth in SEQ ID NO: 401, or a variant thereof,
and/or
the light chain immunoglobulin or variable region thereof comprises:
(a) a CDR-L1 comprising the amino acid sequence set forth in SEQ ID NO: 36, or a variant thereof, a CDR-L2 comprising the amino acid sequence GAS, or a variant thereof, a CDR-L3 comprising the amino acid sequence set forth in SEQ ID NO: 40, or a variant thereof,
(b) a CDR-L1 comprising the amino acid sequence set forth in SEQ ID NO: 56, or a variant thereof, a CDR-L2 comprising the amino acid sequence AAS, or a variant thereof, a CDR-L3 comprising the amino acid sequence set forth in SEQ ID NO: 60, or a variant thereof,
(c) a CDR-L1 comprising the amino acid sequence set forth in SEQ ID NO: 76, or a variant thereof, a CDR-L2 comprising the amino acid sequence AAS, or a variant thereof, a CDR-L3 comprising the amino acid sequence set forth in SEQ ID NO: 80, or a variant thereof,
(d) a CDR-L1 comprising the amino acid sequence set forth in SEQ ID NO: 96, or a variant thereof, a CDR-L2 comprising the amino acid sequence KIS, or a variant thereof, a CDR-L3 comprising the amino acid sequence set forth in SEQ ID NO: 100, or a variant thereof;
(e) a CDR-L1 comprising the amino acid sequence set forth in SEQ ID NO: 116, or a variant thereof, a CDR-L2 comprising the amino acid sequence AAS, or a variant thereof, a CDR-L3 comprising the amino acid sequence set forth in SEQ ID NO: 120, or a variant thereof,
(f) a CDR-L1 comprising the amino acid sequence set forth in SEQ ID NO: 136, or a variant thereof, a CDR-L2 comprising the amino acid sequence GAS, or a variant thereof, a CDR-L3 comprising the amino acid sequence set forth in SEQ ID NO: 140, or a variant thereof,
(g) a CDR-L1 comprising the amino acid sequence set forth in SEQ ID NO: 156, or a variant thereof, a CDR-L2 comprising the amino acid sequence AAS, or a variant thereof, a CDR-L3 comprising the amino acid sequence set forth in SEQ ID NO: 160, or a variant thereof;
(h) a CDR-L1 comprising the amino acid sequence set forth in SEQ ID NO: 176, or a variant thereof, a CDR-L2 comprising the amino acid sequence AAS, or a variant thereof, a CDR-L3 comprising the amino acid sequence set forth in SEQ ID NO: 180, or a variant thereof;
(i) a CDR-L1 comprising the amino acid sequence set forth in SEQ ID NO: 197, or a variant thereof, a CDR-L2 comprising the amino acid sequence AAS, or a variant thereof, a CDR-L3 comprising the amino acid sequence set forth in SEQ ID NO: 201, or a variant thereof;
(j) a CDR-L1 comprising the amino acid sequence set forth in SEQ ID NO: 217, or a variant thereof, a CDR-L2 comprising the amino acid sequence KIS, or a variant thereof, a CDR-L3 comprising the amino acid sequence set forth in SEQ ID NO: 221, or a variant thereof;
(k) a CDR-L1 comprising the amino acid sequence set forth in SEQ ID NO: 237, or a variant thereof, a CDR-L2 comprising the amino acid sequence AAS, or a variant thereof, a CDR-L3 comprising the amino acid sequence set forth in SEQ ID NO: 241, or a variant thereof;
(l) a CDR-L1 comprising the amino acid sequence set forth in SEQ ID NO: 257, or a variant thereof, a CDR-L2 comprising the amino acid sequence AAS, or a variant thereof, a CDR-L3 comprising the amino acid sequence set forth in SEQ ID NO: 261, or a variant thereof,
(m) a CDR-L1 comprising the amino acid sequence set forth in SEQ ID NO: 285, or a variant thereof, a CDR-L2 comprising the amino acid sequence AAS, or a variant thereof, a CDR-L3 comprising the amino acid sequence set forth in SEQ ID NO: 289, or a variant thereof,
(n) a CDR-L1 comprising the amino acid sequence set forth in SEQ ID NO: 305, or a variant thereof, a CDR-L2 comprising the amino acid sequence AAS, or a variant thereof, a CDR-L3 comprising the amino acid sequence set forth in SEQ ID NO: 309, or a variant thereof,
(o) a CDR-L1 comprising the amino acid sequence set forth in SEQ ID NO: 325, or a variant thereof, a CDR-L2 comprising the amino acid sequence AAS, or a variant thereof, a CDR-L3 comprising the amino acid sequence set forth in SEQ ID NO: 329, or a variant thereof,
(p) a CDR-L1 comprising the amino acid sequence set forth in SEQ ID NO: 345, or a variant thereof, a CDR-L2 comprising the amino acid sequence GAS, or a variant thereof, a CDR-L3 comprising the amino acid sequence set forth in SEQ ID NO: 349, or a variant thereof,
(q) a CDR-L1 comprising the amino acid sequence set forth in SEQ ID NO: 365, or a variant thereof, a CDR-L2 comprising the amino acid sequence GAS, or a variant thereof, a CDR-L3 comprising the amino acid sequence set forth in SEQ ID NO: 369, or a variant thereof;
(r) a CDR-L1 comprising the amino acid sequence set forth in SEQ ID NO: 385, or a variant thereof, a CDR-L2 comprising the amino acid sequence GAS, or a variant thereof, a CDR-L3 comprising the amino acid sequence set forth in SEQ ID NO: 389, or a variant thereof;
and/or
(s) a CDR-L1 comprising the amino acid sequence set forth in SEQ ID NO: 405, or a variant thereof, a CDR-L2 comprising the amino acid sequence GAS, or a variant thereof, a CDR-L3 comprising the amino acid sequence set forth in SEQ ID NO: 409, or a variant thereof.

171. The antibody-tethered drug conjugate of claim 133, wherein:
(1) the heavy chain immunoglobulin or variable region thereof comprises a CDR-H1 comprising the amino acid sequence set forth in SEQ ID NO: 28, or a variant thereof, a CDR-H2 comprising the amino acid sequence set forth in SEQ ID NO: 30, or a variant thereof, and a CDR-H3 comprising the amino acid sequence set forth in SEQ ID NO: 32; and
the light chain immunoglobulin or variable region thereof comprises a CDR-L1 comprising the amino acid sequence set forth in SEQ ID NO: 36, or a variant thereof, a CDR-L2 comprising the amino acid sequence GAS, or a variant thereof, and a CDR-L3 comprising the amino acid sequence set forth in SEQ ID NO: 40, or a variant thereof,
(2) the heavy chain immunoglobulin or variable region thereof comprises a CDR-H1 comprising the amino acid sequence set forth in SEQ ID NO: 48, or a variant thereof, a CDR-H2 comprising the amino acid sequence set forth in SEQ ID NO: 50, or a variant thereof, and a CDR-H3 comprising the amino acid sequence set forth in SEQ ID NO: 52; and
the light chain immunoglobulin or variable region thereof comprises a CDR-L1 comprising the amino acid sequence set forth in SEQ ID NO: 56, or a variant thereof, a CDR-L2 comprising the amino acid sequence AAS, or a variant thereof, and a CDR-L3 comprising the amino acid sequence set forth in SEQ ID NO: 60, or a variant thereof,
(3) the heavy chain immunoglobulin or variable region thereof comprises a CDR-H1 comprising the amino acid sequence set forth in SEQ ID NO: 68, or a variant thereof, a CDR-H2 comprising the amino acid sequence set forth in SEQ ID NO: 70, or a variant thereof, and a CDR-H3 comprising the amino acid sequence set forth in SEQ ID NO: 72; and
the light chain immunoglobulin or variable region thereof comprises a CDR-L1 comprising the amino acid sequence set forth in SEQ ID NO: 76, or a variant thereof, a CDR-L2 comprising the amino acid sequence AAS, or a variant thereof, and a CDR-L3 comprising the amino acid sequence set forth in SEQ ID NO: 80, or a variant thereof,
(4) the heavy chain immunoglobulin or variable region thereof comprises a CDR-H1 comprising the amino acid sequence set forth in SEQ ID NO: 88, or a variant thereof, a CDR-H2 comprising the amino acid sequence set forth in SEQ ID NO: 90, or a variant thereof, and a CDR-H3 comprising the amino acid sequence set forth in SEQ ID NO: 92; and
the light chain immunoglobulin or variable region thereof comprises a CDR-L1 comprising the amino acid sequence set forth in SEQ ID NO: 96, or a variant thereof, a CDR-L2 comprising the amino acid sequence KIS, or a variant thereof; and a CDR-L3 comprising the amino acid sequence set forth in SEQ ID NO: 100, or a variant thereof,
(5) the heavy chain immunoglobulin or variable region thereof comprises a CDR-H1 comprising the amino acid sequence set forth in SEQ ID NO: 108, or a variant thereof, a CDR-H2 comprising the amino acid sequence set forth in SEQ ID NO: 110, or a variant thereof, and a CDR-H3 comprising the amino acid sequence set forth in SEQ ID NO: 112; and
the light chain immunoglobulin or variable region thereof comprises a CDR-L1 comprising the amino acid sequence set forth in SEQ ID NO: 116, or a variant thereof, a CDR-L2 comprising the amino acid sequence AAS, or a variant thereof; and a CDR-L3 comprising the amino acid sequence set forth in SEQ ID NO: 120, or a variant thereof,
(6) the heavy chain immunoglobulin or variable region thereof comprises a CDR-H1 comprising the amino acid sequence set forth in SEQ ID NO: 128, or a variant thereof, a CDR-H2 comprising the amino acid sequence set forth in SEQ ID NO: 130, or a variant thereof, and a CDR-H3 comprising the amino acid sequence set forth in SEQ ID NO: 132; and
the light chain immunoglobulin or variable region thereof comprises a CDR-L1 comprising the amino acid sequence set forth in SEQ ID NO: 136, or a variant thereof, a CDR-L2 comprising the amino acid sequence GAS, or a variant thereof; and a CDR-L3 comprising the amino acid sequence set forth in SEQ ID NO: 140, or a variant thereof,
(7) the heavy chain immunoglobulin or variable region thereof comprises a CDR-H1 comprising the amino acid sequence set forth in SEQ ID NO: 148, or a variant thereof, a CDR-H2 comprising the amino acid sequence set forth in SEQ ID NO: 150, or a variant thereof, and a CDR-H3 comprising the amino acid sequence set forth in SEQ ID NO: 152; and
the light chain immunoglobulin or variable region thereof comprises a CDR-L1 comprising the amino acid sequence set forth in SEQ ID NO: 156, or a variant thereof, a CDR-L2 comprising the amino acid sequence AAS, or a variant thereof; and a CDR-L3 comprising the amino acid sequence set forth in SEQ ID NO: 160, or a variant thereof,
(8) the heavy chain immunoglobulin or variable region thereof comprises a CDR-H1 comprising the amino acid sequence set forth in SEQ ID NO: 168, or a variant thereof, a CDR-H2 comprising the amino acid sequence set forth in SEQ ID NO: 170, or a variant thereof, and a CDR-H3 comprising the amino acid sequence set forth in SEQ ID NO: 172;
the light chain immunoglobulin or variable region thereof comprises a CDR-L1 comprising the amino acid sequence set forth in SEQ ID NO: 176, or a variant thereof, a CDR-L2 comprising the amino acid sequence AAS, or a variant thereof; and a CDR-L3 comprising the amino acid sequence set forth in SEQ ID NO: 180, or a variant thereof,
(9) the heavy chain immunoglobulin or variable region thereof comprises a CDR-H1 comprising the amino acid sequence set forth in SEQ ID NO: 189, or a variant thereof, a CDR-H2 comprising the amino acid sequence set forth in SEQ ID NO: 191, or a variant thereof, a CDR-H3 comprising the amino acid sequence set forth in SEQ ID NO: 193, or a variant thereof, and the light chain immunoglobulin or variable region thereof comprises a CDR-L1 comprising the amino acid sequence set forth in SEQ ID NO: 197, or a variant thereof, a CDR-L2 comprising the amino acid sequence AAS, or a variant thereof; a CDR-L3 comprising the amino acid sequence set forth in SEQ ID NO: 201, or a variant thereof,
(10) the heavy chain immunoglobulin or variable region thereof comprises a CDR-H1 comprising the amino acid sequence set forth in SEQ ID NO: 209, or a variant thereof, a CDR-H2 comprising the amino acid sequence set forth in SEQ ID NO: 211, or a variant thereof, a CDR-H3 comprising the amino acid sequence set forth in SEQ ID NO: 213, or a variant thereof, and the light chain immunoglobulin or variable region thereof comprises a CDR-L1 comprising the amino acid sequence set forth in SEQ ID NO: 217, or a variant thereof, a CDR-L2 comprising the amino acid sequence KIS, or a variant thereof, a CDR-L3 comprising the amino acid sequence set forth in SEQ ID NO: 221, or a variant thereof,
(11) the heavy chain immunoglobulin or variable region thereof comprises a CDR-H1 comprising the amino acid sequence set forth in SEQ ID NO: 229, or a variant thereof, a CDR-H2 comprising the amino acid sequence set forth in SEQ ID NO: 231, or a variant thereof, a CDR-H3 comprising the amino acid sequence set forth in SEQ ID NO: 233, or a variant thereof, and the light chain immunoglobulin or variable region thereof comprises a CDR-L1 comprising the amino acid sequence set forth in SEQ ID NO: 237, or a variant thereof, a CDR-L2 comprising the amino acid sequence AAS, or a variant thereof, a CDR-L3 comprising the amino acid sequence set forth in SEQ ID NO: 241, or a variant thereof,
(12) the heavy chain immunoglobulin or variable region thereof comprises a CDR-H1 comprising the amino acid sequence set forth in SEQ ID NO: 249, or a variant thereof, a CDR-H2 comprising the amino acid sequence set forth in SEQ ID NO: 251, or a variant thereof, a CDR-H3 comprising the amino acid sequence set forth in SEQ ID NO: 253, or a variant thereof, and the light chain immunoglobulin or variable region thereof comprises a CDR-L1 comprising the amino acid sequence set forth in SEQ ID NO: 257, or a variant thereof, a CDR-L2 comprising the amino acid sequence AAS, or a variant thereof, a CDR-L3 comprising the amino acid sequence set forth in SEQ ID NO: 261, or a variant thereof,
(13) the heavy chain immunoglobulin or variable region thereof comprises a CDR-H1 comprising the amino acid sequence set forth in SEQ ID NO: 277, or a variant thereof, a CDR-H2 comprising the amino acid sequence set forth in SEQ ID NO: 279, or a variant thereof, a CDR-H3 comprising the amino acid sequence set forth in SEQ ID NO: 281, or a variant thereof, and the light chain immunoglobulin or variable region thereof comprises a CDR-L1 comprising the amino acid sequence set forth in SEQ ID NO: 285, or a variant thereof, a CDR-L2 comprising the amino acid sequence AAS, or a variant thereof, a CDR-L3 comprising the amino acid sequence set forth in SEQ ID NO: 289, or a variant thereof,
(14) the heavy chain immunoglobulin or variable region thereof comprises a CDR-H1 comprising the amino acid sequence set forth in SEQ ID NO: 297, or a variant thereof, a CDR-H2 comprising the amino acid sequence set forth in SEQ ID NO: 299, or a variant thereof, a CDR-H3 comprising the amino acid sequence set forth in SEQ ID NO: 301, or a variant thereof, and the light chain immunoglobulin or variable region thereof comprises a CDR-L1 comprising the amino acid sequence set forth in SEQ ID NO: 305, or a variant thereof, a CDR-L2 comprising the amino acid sequence AAS, or a variant thereof, a CDR-L3 comprising the amino acid sequence set forth in SEQ ID NO: 309, or a variant thereof,
(15) the heavy chain immunoglobulin or variable region thereof comprises a CDR-H1 comprising the amino acid sequence set forth in SEQ ID NO: 317, or a variant thereof, a CDR-H2 comprising the amino acid sequence set forth in SEQ ID NO: 319, or a variant thereof, a CDR-H3 comprising the amino acid sequence set forth in SEQ ID NO: 321, or a variant thereof, and the light chain immunoglobulin or variable region thereof comprises a CDR-L1 comprising the amino acid sequence set forth in SEQ ID NO: 325, or a variant thereof, a CDR-L2 comprising the amino acid sequence AAS, or a variant thereof, a CDR-L3 comprising the amino acid sequence set forth in SEQ ID NO: 329, or a variant thereof,
(16) the heavy chain immunoglobulin or variable region thereof comprises a CDR-H1 comprising the amino acid sequence set forth in SEQ ID NO: 337, or a variant thereof, a CDR-H2 comprising the amino acid sequence set forth in SEQ ID NO: 339, or a variant thereof, a CDR-H3 comprising the amino acid sequence set forth in SEQ ID NO: 341, or a variant thereof, and the light chain immunoglobulin or variable region thereof comprises a CDR-L1 comprising the amino acid sequence set forth in SEQ ID NO: 345, or a variant thereof, a CDR-L2 comprising the amino acid sequence GAS, or a variant thereof, a CDR-L3 comprising the amino acid sequence set forth in SEQ ID NO: 349, or a variant thereof,
(17) the heavy chain immunoglobulin or variable region thereof comprises a CDR-H1 comprising the amino acid sequence set forth in SEQ ID NO: 357, or a variant thereof, a CDR-H2 comprising the amino acid sequence set forth in SEQ ID NO: 359, or a variant thereof, a CDR-H3 comprising the amino acid sequence set forth in SEQ ID NO: 361, or a variant thereof, and the light chain immunoglobulin or variable region thereof comprises a CDR-L1 comprising the amino acid sequence set forth in SEQ ID NO: 365, or a variant thereof, a CDR-L2 comprising the amino acid sequence GAS, or a variant thereof, a CDR-L3 comprising the amino acid sequence set forth in SEQ ID NO: 369, or a variant thereof,
(18) the heavy chain immunoglobulin or variable region thereof comprises a CDR-H1 comprising the amino acid sequence set forth in SEQ ID NO: 377, or a variant thereof, a CDR-H2 comprising the amino acid sequence set forth in SEQ ID NO: 379, or a variant thereof, a CDR-H3 comprising the amino acid sequence set forth in SEQ ID NO: 381, or a variant thereof, and the light chain immunoglobulin or variable region thereof comprises a CDR-L1 comprising the amino acid sequence set forth in SEQ ID NO: 385, or a variant thereof, a CDR-L2 comprising the amino acid sequence GAS, or a variant thereof, a CDR-L3 comprising the amino acid sequence set forth in SEQ ID NO: 389, or a variant thereof, or
(19) the heavy chain immunoglobulin or variable region thereof comprises a CDR-H1 comprising the amino acid sequence set forth in SEQ ID NO: 397, or a variant thereof, a CDR-H2 comprising the amino acid sequence set forth in SEQ ID NO: 399, or a variant thereof, a CDR-H3 comprising the amino acid sequence set forth in SEQ ID NO: 401, or a variant thereof, and the light chain immunoglobulin or variable region thereof comprises a CDR-L1 comprising the amino acid sequence set forth in SEQ ID NO: 405, or a variant thereof, a CDR-L2 comprising the amino acid sequence GAS, or a variant thereof, a CDR-L3 comprising the amino acid sequence set forth in SEQ ID NO: 409, or a variant thereof.

172. The antibody-tethered drug conjugate of claim 133, wherein:
(a) the heavy chain immunoglobulin variable region comprises the amino acid sequence
(SEQ ID NO: 26)
EVQLVESGGGLVKPGGSLRLSCAASGFIFSRYSMNWVRQAPGKGLEWVSSMSSNSKNT
YYADSVKGRFTISRDNAKNSLFLQMNTLRAEDTAVYYCARDGYTLRAFDIWGQGTMVTVSS,
and the light chain immunoglobulin variable region comprises the
amino acid sequence
(SEQ ID NO: 34)
EIVLTQSPGTLSLSPGERDTLSCRASQSIAGRYVAWYQQKPGQAPRLLIYGASSRATGIPD
RFSGSGSGTDFTLTISRLEPEDFAVYYCQQYGSSPWTFGQGTKVEIK;
(b) the heavy chain immunoglobulin variable region comprises the amino acid sequence
(SEQ ID NO: 46)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTL
VTVSS,
and the light chain immunoglobulin variable region comprises the
amino acid sequence
(SEQ ID NO: 420)
DIQMTQSPSSVSASVGDRVTITCRASQGINSWLAWYQQKPGKAPKLLIYAASSLQSGVPS
RFSGSGSGTDFTLTISSLQPEDFATYYCHQADSFPYTFGQGTKLEIK;
(c) the heavy chain immunoglobulin variable region comprises the amino acid sequence
(SEQ ID NO: 66)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTL
VTVSS,
and the light chain immunoglobulin variable region comprises the
amino acid sequence
(SEQ ID NO: 421)
DIQMTQSPSSVSASVGDRVTITCRASQGINSWLAWYQQKPGKAPKLLIYAASSLESGVPS
RFSGSGSGTDFTLTISSLQPEDFATYYCHQADSFPYTFGQGTKLEIK;
(d) the heavy chain immunoglobulin variable region comprises the amino acid sequence
(SEQ ID NO: 86)
QVQLVESGGGVGQPGRSLRLSCAASGFTFSRNAMHWVRQAPGKGLEWVAVISYDGSN
KHYADSVKGRFTISRDNSKNTLYLEMNSLRVEDTAVYYCAKGGIPFDYWGQGTLVTVSS,
and the light chain immunoglobulin variable region comprises the amino
acid sequence
(SEQ ID NO: 422)
DIVMTQSPLSSPVTLGQPASISCRSSQSLVHFDGNTYLSWLHQRPGQPPRLLIYKISNRFS
GVPDRFSGSGAGTDFTLKISRVEPEDVGVYYCMHATQFPYTFGQGTKLEIK;
(e) the heavy chain immunoglobulin variable region comprises the amino acid sequence
(SEQ ID NO: 106)
QVQLVESGGGVVQPARSLRLSCAASGFAFSRSAMHWVRQAPGKGLEWVAVISYDGSN
KYYTDSVKGRFTISRDNSKNTLYLQMNTLRAEDTALYYCAKMYTTMDSFDYWGQGTLVTVSS,
and the light chain immunoglobulin variable region comprises the
amino acid sequence
(SEQ ID NO: 423)
DIQLTQSPSFLSASVGDRVTITCWASQGISSYLAWYQQKPGKAPKLLIYAASTLQSGVPS
RFSGSGSGTEFTLTISSLQPEDFALYYCQQLNSYPRTFGQGTKVEIK;
(f) the heavy chain immunoglobulin variable region comprises the amino acid sequence
(SEQ ID NO: 424)
EVQLVESGGGLVKPGGSLRLSCAASGFIFSRYSMNWVRQAPGKGLEWVSSMSSNSKNT
YYADSVKGRFTISRDNAKNSLFLQMNTLRAEDTAVYYCARDGYTLRAFDIWGQGTMV
TVSS,
and the light chain immunoglobulin variable region comprises the
amino acid sequence
(SEQ ID NO: 134)
EIVLTQSPGTLSLSPGERDTLSCRASQSIAGRYVAWYQQKPGQAPRLLIYGASSRATGIPD
RFSGSGSGTDFTLTISRLEPEDFA VYYCQQYGSSPWTFGQGTKVEIK;
(g) the heavy chain immunoglobulin variable region comprises the amino acid sequence
(SEQ ID NO: 146)
QVQLVESGGGVVQPGRSLRLSCAASGFTFSGYGIHWVRQAPGKGLVWVAVIWYDGSF
KYYADSVKGRFTISRDNSKNTLYLQMNSLRAEDTAVYYCARGDSSSSGRYYYYGMDV
WGQGTTVTVSS,
and the light chain immunoglobulin variable region
comprises the amino acid sequence
(SEQ ID NO: 425)
DIQMTQSPSSLSASVGDRVTITCRASQGIRNDLGWYQQKPGTAPKRLIFAASSLQSGVPS
RFSGSGSGTEFTLTISSLQPEDFATYYCLQHNNYPPTFGGGTKVEIK;
(h) the heavy chain immunoglobulin variable region comprises the amino acid sequence
(SEQ ID NO: 166)
EVQLVESGGGLVQPGGSLKLSCAASGFTFSGSAMHWVRQASGKGLEWVGRITSKANSY
ATAYDASVKGRFTISRDDSKNTAYLQMNSLKTEDTAVYYCTRQRFLEFLFLDYWGQGTLVTVSS,
and the light chain immunoglobulin variable region comprises the
amino acid sequence
(SEQ ID NO: 426)
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPITFGQGTRLEIK;
(i) the heavy chain immunoglobulin variable region comprises the amino acid sequence
(SEQ ID NO: 427)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTLVTVSS,
and the light chain immunoglobulin variable region comprises the
amino acid sequence
(SEQ ID NO: 195)
DIQMTQSPSSVSASVGDRVTITCRASQGINSWLAWYQQKPGKAPKLLIYAASSLQSGVPS
RFSGSGSGTDFTLTISSLQPEDFATYYCHQADSFPYTFGQGTKLEIK;
(j) the heavy chain immunoglobulin variable region comprises the amino acid sequence
(SEQ ID NO: 428)
QVQLVESGGGVGQPGRSLRLSCAASGFTFSRNAMHWVRQAPGKGLEWVAVISYDGSN
KHYADSVKGRFTISRDNSKNTLYLEMNSLRVEDTAVYYCAKGGIPFDYWGQGTLVTVSS,
and the light chain immunoglobulin variable region comprises the amino acid sequence
(SEQ ID NO: 215)
DIVMTQSPLSSPVTLGQPASISCRSSQSLVHFDGNTYLSWLHQRPGQPPRLLIYKISNRFS
GVPDRFSGSGAGTDFTLKISRVEPEDVGVYYCMHATQFPYTFGQGTKLEIK;
(k) the heavy chain immunoglobulin variable region comprises the amino acid sequence
(SEQ ID NO: 429)
QVQLVESGGGVVQPARSLRLSCAASGFAFSRSAMHWVRQAPGKGLEWVAVISYDGSN
KYYTDSVKGRFTISRDNSKNTLYLQMNTLRAEDTALYYCAKMYTTMDSFDYWGQGTL
VTVSS,
and the light chain immunoglobulin variable region comprises the
amino acid sequence
(SEQ ID NO: 235)
DIQLTQSPSFLSASVGDRVTITCWASQGISSYLAWYQQKPGKAPKLLIYAASTLQSGVPS
RFSGSGSGTEFTLTISSLQPEDFALYYCQQLNSYPRTFGQGTKVEIK;
(l) the heavy chain immunoglobulin variable region comprises the amino acid sequence
(SEQ ID NO: 430)
EVQLVESGGGLVQPGGSLKLSCAASGFTFSGSAMHWVRQASGKGLEWVGRITSKANSY
ATAYDASVKGRFTISRDDSKNTAYLQMNSLKTEDTAVYYCTRQRFLEFLFLDYWGQGT
LVTVSS,
and the light chain immunoglobulin variable region comprises the amino acid sequence
(SEQ ID NO: 255)
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPITFGQGTRLEIK;
(m) the heavy chain immunoglobulin variable region comprises the amino acid sequence
(SEQ ID NO: 275)
EVQLVESGGGLVQPGGSLRLSCAASGFTFSRYWMTWVRQAPGKGLEWVANIKQDGSG
KNYVDSVMGRYTISRDNAKNSLYLQMNSLRAEDTAVYYCARWIAPDFPGMDVWGQG
TTVTVSS,
and the light chain immunoglobulin variable region comprises
the amino acid sequence
(SEQ ID NO: 431)
DIQLTQSPSFLSASVGDRVTITCWASQGISSYLAWYQQKPGKAPKLLIYAASTLQSGVPS
RFSGSGSGTEFTLTISSLQPADFATYYCQQLNSYPLTFGGGTKVEIK;
(n) the heavy chain immunoglobulin variable region comprises the amino acid sequence
(SEQ ID NO: 295)
EVQLVESGGGLVQPGGSLKLSCAASGFTFSGSAMHWVRQASGKGLEWVGRITSKANSY
ATAYDASVKGRFTISRDDSKNTAYLQMNSLKTEDTAVYYCTRQRFLEFLFLDYWGQGTLVTVSS,
and the light chain immunoglobulin variable region comprises the
amino acid sequence
(SEQ ID NO: 432)
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPITFGQGTRLEIK;
(o) the heavy chain immunoglobulin variable region comprises the amino acid sequence
(SEQ ID NO: 433)
QVQLVESGGGVVQPGRSLRLSCAASGFTFSGYGIHWVRQAPGKGLVWVAVIWYDGSF
KYYADSVKGRFTISRDNSKNTLYLQMNSLRAEDTAVYYCARGDSSSSGRYYYYGMDV
WGQGTTVTVSS,
and the light chain immunoglobulin variable region
comprises the amino acid sequence
(SEQ ID NO: 323)
DIQMTQSPSSLSASVGDRVTITCRASQGIRNDLGWYQQKPGTAPKRLIFAASSLQSGVPS
RFSGSGSGTEFTLTISSLQPEDFATYYCLQHNNYPPTFGGGTKVEIK;
(p) the heavy chain immunoglobulin variable region comprises the amino acid sequence
(SEQ ID NO: 434)
QVQLQQWGAGLLKPSETLSLTCAVYGGSFSGYYWSWIRQPPGKGLEWIGEINHAGSTN
YNPSLKSRITISVDTSKNQFSLKLSSVTAADTAVYYCARGWYYGSGSYHRNWFDPWGQ
GTLVTVSS,
and the light chain immunoglobulin variable region comprises
the amino acid sequence
(SEQ ID NO: 343)
EIVLTQSPGTLSLSPGERATLSCRASQSVYYSYLAWYQQKPGQAPRLLIYGASNRATGIP
DRFSGSGSGTDFTLTISRLEPEDFAVYYCQQYGNSPWTFGQGTKVEIK;
(q) the heavy chain immunoglobulin variable region comprises the amino acid sequence
(SEQ ID NO: 355)
QVQLQQWGAGLLKPSETLSLTCAVYGGSFSGYYWSWIRQPPGKGLEWIGEINHAGSTN
YNPSLKSRITISVDTSKNQFSLKLSSVTAADTAVYYCARGWYYGSGSYHRNWFDPWGQ
GTLVTVSS,
and the light chain immunoglobulin variable region comprises
the amino acid sequence
(SEQ ID NO: 435)
EIVLTQSPGTLSLSPGERATLSCRASQSVYYSYLAWYQQKPGQAPRLLIYGASNRATGIP
DRFSGSGSGTDFTLTISRLEPEDFAVYYCQQYGNSPWTFGQGTKVEIK;
(r) the heavy chain immunoglobulin variable region comprises the amino acid sequence
(SEQ ID NO: 436)
EVQVVESGGGLVQPGRSLRLSCTASGFTFDDYAMFWVRQGPGKGLEWVSGISWNSGSI
GYADSVKGRFTTSRDNAKNSLYLQMNSLRTEDTALYYCAKDYRPRSGNHYNNYGMD
VWGPGTTVTVSS,
and the light chain immunoglobulin variable region
comprises the amino acid sequence
(SEQ ID NO: 383)
EIVLTQSPGTLSLSPGERATLSCRASQSFRGNYLAWYQQKPGQAPRLLIYGASSRATGIP
DRFRGSGSGTDFTLTISRLEPEDFA VYYCHQYGRSPWTFGQGTKVEIK;
and/or
(s) the heavy chain immunoglobulin variable region comprises the amino acid sequence
(SEQ ID NO: 395)
EVQVVESGGGLVQPGRSLRLSCTASGFTFDDYAMFWVRQGPGKGLEWVSGISWNSGSI
GYADSVKGRFTTSRDNAKNSLYLQMNSLRTEDTALYYCAKDYRPRSGNHYNNYGMD
VWGPGTTVTVSS,
and the light chain immunoglobulin variable region
comprises the amino acid sequence
(SEQ ID NO: 437)
EIVLTQSPGTLSLSPGERATLSCRASQSFRGNYLAWYQQKPGQAPRLLIYGASSRATGIP
DRFRGSGSGTDFTLTISRLEPEDFA VYYCHQYGRSPWTFGQGTKVEIK.

173. The antibody-tethered drug conjugate of claim 133, wherein:
(a) the heavy chain immunoglobulin comprises the amino acid sequence
(SEQ ID NO: 42)
EVQLVESGGGLVKPGGSLRLSCAASGFIFSRYSMNWVRQAPGKGLEWVSSMSSNSKNT
YYADSVKGRFTISRDNAKNSLFLQMNTLRAEDTAVYYCARDGYTLRAFDIWGQGTMV
TVSSASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK,
and the light chain immunoglobulin comprises the amino acid sequence
(SEQ ID NO: 44)
LLQGSGEIVLTQSPGTLSLSPGERDTLSCRASQSIAGRYVAWYQQKPGQAPRLLIYGASS
RATGIPDRFSGSGSGTDFTLTISRLEPEDFAVYYCQQYGSSPWTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC;
(b) the heavy chain immunoglobulin comprises the amino acid sequence
(SEQ ID NO: 62)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTL
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK,
and the light chain immunoglobulin comprises the amino acid sequence
(SEQ ID NO: 64)
LLQGSGDIQMTQSPSSVSASVGDRVTITCRASQGINSWLAWYQQKPGKAPKLLIYAASS
LQSGVPSRFSGSGSGTDFTLTISSLQPEDFATYYCHQADSFPYTFGQGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC;
(c) the heavy chain immunoglobulin comprises the amino acid sequence
(SEQ ID NO: 82)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTL
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK,
and the light chain immunoglobulin comprises the amino acid sequence
(SEQ ID NO: 84)
LLQGSGDIQMTQSPSSVSASVGDRVTITCRASQGINSWLAWYQQKPGKAPKLLIYAASS
LESGVPSRFSGSGSGTDFTLTISSLQPEDFATYYCHQADSFPYTFGQGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC;
(d) the heavy chain immunoglobulin comprises the amino acid sequence
(SEQ ID NO: 102)
QVQLVESGGGVGQPGRSLRLSCAASGFTFSRNAMHWVRQAPGKGLEWVAVISYDGSN
KHYADSVKGRFTISRDNSKNTLYLEMNSLRVEDTAVYYCAKGGIPFDYWGQGTLVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGK,
and the light chain immunoglobulin comprises the amino acid sequence
(SEQ ID NO: 104)
LLQGSGDIVMTQSPLSSPVTLGQPASISCRSSQSLVHFDGNTYLSWLHQRPGQPPRLLIYK
ISNRFSGVPDRFSGSGAGTDFTLKISRVEPEDVGVYYCMHATQFPYTFGQGTKLEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC;
(e) the heavy chain immunoglobulin comprises the amino acid sequence
(SEQ ID NO: 122)
QVQLVESGGGVVQPARSLRLSCAASGFAFSRSAMHWVRQAPGKGLEWVAVISYDGSN
KYYTDSVKGRFTISRDNSKNTLYLQMNTLRAEDTALYYCAKMYTTMDSFDYWGQGTL
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK,
and the light chain immunoglobulin comprises the amino acid sequence
(SEQ ID NO: 124)
LLQGSGDIQLTQSPSFLSASVGDRVTITCWASQGISSYLAWYQQKPGKAPKLLIYAASTL
QSGVPSRFSGSGSGTEFTLTISSLQPEDFALYYCQQLNSYPRTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC;
(f) the heavy chain immunoglobulin comprises the amino acid sequence
(SEQ ID NO: 142)
LLQGSGEVQLVESGGGLVKPGGSLRLSCAASGFIFSRYSMNWVRQAPGKGLEWVSSMS
SNSKNTYYADSVKGRFTISRDNAKNSLFLQMNTLRAEDTAVYYCARDGYTLRAFDIWG
QGTMVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGK,
and the light chain immunoglobulin comprises the amino acid sequence
(SEQ ID NO: 144)
EIVLTQSPGTLSLSPGERDTLSCRASQSIAGRYVAWYQQKPGQAPRLLIYGASSRATGIPD
RFSGSGSGTDFTLTISRLEPEDFAVYYCQQYGSSPWTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC;
(g) the heavy chain immunoglobulin comprises the amino acid sequence
(SEQ ID NO: 162)
QVQLVESGGGVVQPGRSLRLSCAASGFTFSGYGIHWVRQAPGKGLVWVAVIWYDGSF
KYYADSVKGRFTISRDNSKNTLYLQMNSLRAEDTAVYYCARGDSSSSGRYYYYGMDV
WGQGTTVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK,
and the light chain immunoglobulin comprises the amino acid sequence
(SEQ ID NO: 164)
LLQGSGDIQMTQSPSSLSASVGDRVTITCRASQGIRNDLGWYQQKPGTAPKRLIFAASSL
QSGVPSRFSGSGSGTEFTLTISSLQPEDFATYYCLQHNNYPPTFGGGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC;
(h) the heavy chain immunoglobulin comprises the amino acid sequence
(SEQ ID NO: 182)
EVQLVESGGGLVQPGGSLKLSCAASGFTFSGSAMHWVRQASGKGLEWVGRITSKANSY
ATAYDASVKGRFTISRDDSKNTAYLQMNSLKTEDTAVYYCTRQRFLEFLFLDYWGQGT
LVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK,
and the light chain immunoglobulin comprises the amino acid sequence
(SEQ ID NO: 184)
LLQGSGDIQMTQSPSSLSASVGDRVTITCRASQSISSYLNWYQQKPGKAPKLLIYAASSL
QSGVPSRFSGSGSGTDFTLTISSLQPEDFATYYCQQSYSTPPITFGQGTRLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC;
(i) the heavy chain immunoglobulin comprises the amino acid sequence
(SEQ ID NO: 203)
LLQGSGEVQLVESGGGLVQPGGSLRLSCAASGFTFSSYAMSWVRQAPGKGLEWVSAIS
GSGGSTYYADSVKGRFTISRDNSKNTLYLQMNSLRAEDTAVYYCAKGLIAPRPMGFDY
WGQGTLVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK,
and the light chain immunoglobulin comprises the amino acid sequence
(SEQ ID NO: 205)
DIQMTQSPSSVSASVGDRVTITCRASQGINSWLAWYQQKPGKAPKLLIYAASSLQSGVPS
RFSGSGSGTDFTLTISSLQPEDFATYYCHQADSFPYTFGQGTKLEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC;
(j) the heavy chain immunoglobulin comprises the amino acid sequence
(SEQ ID NO: 223)
LLQGSGQVQLVESGGGVGQPGRSLRLSCAASGFTFSRNAMHWVRQAPGKGLEWVAVI
SYDGSNKHYADSVKGRFTISRDNSKNTLYLEMNSLRVEDTAVYYCAKGGIPFDYWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK,
and the light chain immunoglobulin comprises the amino acid sequence
(SEQ ID NO: 225)
DIVMTQSPLSSPVTLGQPASISCRSSQSLVHFDGNTYLSWLHQRPGQPPRLLIYKISNRFS
GVPDRFSGSGAGTDFTLKISRVEPEDVGVYYCMHATQFPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC;
(k) the heavy chain immunoglobulin comprises the amino acid sequence
(SEQ ID NO: 243)
LLQGSGQVQLVESGGGVVQPARSLRLSCAASGFAFSRSAMHWVRQAPGKGLEWVAVIS
YDGSNKYYTDSVKGRFTISRDNSKNTLYLQMNTLRAEDTALYYCAKMYTTMDSFDYW
GQGTLVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGK,
and the light chain immunoglobulin comprises the amino acid sequence
(SEQ ID NO: 245)
DIQLTQSPSFLSASVGDRVTITCWASQGISSYLAWYQQKPGKAPKLLIYAASTLQSGVPS
RFSGSGSGTEFTLTISSLQPEDFALYYCQQLNSYPRTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC;
(l) the heavy chain immunoglobulin comprises the amino acid sequence
(SEQ ID NO: 263)
LLQGSGEVQLVESGGGLVQPGGSLKLSCAASGFTFSGSAMHWVRQASGKGLEWVGRIT
SKANSYATAYDASVKGRFTISRDDSKNTAYLQMNSLKTEDTAVYYCTRQRFLEFLFLD
YWGQGTLVTVSSASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKVDKRVESKYGPPCP
PCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK,
and the light chain immunoglobulin comprises the amino acid sequence
(SEQ ID NO: 265)
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPITFGQGTRLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC;
(m) the heavy chain immunoglobulin comprises the amino acid sequence
(SEQ ID NO: 267)
LLQGSGQVTLKESGPGILQPSQTLSLTCSFSGFSLSTSGTGVGWIRQPSGKGLEWLSHIW
WDDVKRYNPALKSRLTISRDTSYSQVFLRIASVDTADTATYYCARILDGTGPMDYWGQ
GTSVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAP
EFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK,
and the light chain immunoglobulin comprises the amino acid sequence
(SEQ ID NO: 269)
QIVLTQSPAIMSASPGEKVTMTCSASSRVTYMHWYQQRSGTSPKRWIYDTSKLASGVPA
RFSGSGSGTSYSLTISSMEAEDAATYYCQQWGNNPQYTFGGGTRLEIKRRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC;
(n) the heavy chain immunoglobulin comprises the amino acid sequence
(SEQ ID NO: 271)
QVTLKESGPGILQPSQTLSLTCSFSGFSLSTSGTGVGWIRQPSGKGLEWLSHIWWDDVKR
YNPALKSRLTISRDTSYSQVFLRIASVDTADTATYYCARILDGTGPMDYWGQGTSVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGK,
and the light chain immunoglobulin comprises the amino acid sequence
(SEQ ID NO: 273)
LLQGSGQIVLTQSPAIMSASPGEKVTMTCSASSRVTYMHWYQQRSGTSPKRWIYDTSKL
ASGVPARFSGSGSGTSYSLTISSMEAEDAATYYCQQWGNNPQYTFGGGTRLEIKRRTVA
APSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC;
(o) the heavy chain immunoglobulin comprises the amino acid sequence
(SEQ ID NO: 291)
EVQLVESGGGLVQPGGSLRLSCAASGFTFSRYWMTWVRQAPGKGLEWVANIKQDGSG
KNYVDSVMGRYTISRDNAKNSLYLQMNSLRAEDTAVYYCARWIAPDFPGMDVWGQG
TTVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK,
and the light chain immunoglobulin comprises the amino acid sequence
(SEQ ID NO: 293)
LLQGSGDIQLTQSPSFLSASVGDRVTITCWASQGISSYLAWYQQKPGKAPKLLIYAASTL
QSGVPSRFSGSGSGTEFTLTISSLQPADFATYYCQQLNSYPLTFGGGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC;
(p) the heavy chain immunoglobulin comprises the amino acid sequence
(SEQ ID NO: 311)
EVQLVESGGGLVQPGGSLKLSCAASGFTFSGSAMHWVRQASGKGLEWVGRITSKANSY
ATAYDASVKGRFTISRDDSKNTAYLQMNSLKTEDTAVYYCTRQRFLEFLFLDYWGQGT
LVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK,
and the light chain immunoglobulin comprises the amino acid sequence
(SEQ ID NO: 313)
LLQGSGDIQMTQSPSSLSASVGDRVTITCRASQSISSYLNWYQQKPGKAPKLLIYAASSL
QSGVPSRFSGSGSGTDFTLTISSLQPEDFATYYCQQSYSTPPITFGQGTRLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC;
(q) the heavy chain immunoglobulin comprises the amino acid sequence
(SEQ ID NO: 331)
LLQGSGQVQLVESGGGVVQPGRSLRLSCAASGFTFSGYGIHWVRQAPGKGLVWVAVI
WYDGSFKYYADSVKGRFTISRDNSKNTLYLQMNSLRAEDTAVYYCARGDSSSSGRYYY
YGMDVWGQGTTVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKY
GPPCPPCPAPEFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK,
and the light chain immunoglobulin comprises the amino acid sequence
(SEQ ID NO: 333)
DIQMTQSPSSLSASVGDRVTITCRASQGIRNDLGWYQQKPGTAPKRLIFAASSLQSGVPS
RFSGSGSGTEFTLTISSLQPEDFATYYCLQHNNYPPTFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC;
(r) the heavy chain immunoglobulin comprises the amino acid sequence
(SEQ ID NO: 351)
LLQGSGQVQLQQWGAGLLKPSETLSLTCAVYGGSFSGYYWSWIRQPPGKGLEWIGEIN
HAGSTNYNPSLKSRITISVDTSKNQFSLKLSSVTAADTAVYYCARGWYYGSGSYHRNW
FDPWGQGTLVTVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGPP
CPPCPAPEFLGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK,
and the light chain immunoglobulin comprises the amino acid sequence
(SEQ ID NO: 353)
EIVLTQSPGTLSLSPGERATLSCRASQSVYYSYLAWYQQKPGQAPRLLIYGASNRATGIP
DRFSGSGSGTDFTLTISRLEPEDFAVYYCQQYGNSPWTFGQGTKVEIKRTVAAPSVFIFPP
SDEQLKSGTASVVCLLNNFYPREAKVQWKVDNALQSGNSQESVTEQDSKDSTYSLSST
LTLSKADYEKHKVYACEVTHQGLSSPVTKSFNRGEC;
(s) the heavy chain immunoglobulin comprises the amino acid sequence
(SEQ ID NO: 371)
QVQLQQWGAGLLKPSETLSLTCAVYGGSFSGYYWSWIRQPPGKGLEWIGEINHAGSTN
YNPSLKSRITISVDTSKNQFSLKLSSVTAADTAVYYCARGWYYGSGSYHRNWFDPWGQ
GTLVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAP
EFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK,
and the light chain immunoglobulin comprises the amino acid sequence
(SEQ ID NO: 373)
LLQGSGEIVLTQSPGTLSLSPGERATLSCRASQSVYYSYLAWYQQKPGQAPRLLIYGASN
RATGIPDRFSGSGSGTDFTLTISRLEPEDFAVYYCQQYGNSPWTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC;
(t) the heavy chain immunoglobulin comprises the amino acid sequence
(SEQ ID NO: 391)
LLQGSGEVQVVESGGGLVQPGRSLRLSCTASGFTFDDYAMFWVRQGPGKGLEWVSGIS
WNSGSIGYADSVKGRFTTSRDNAKNSLYLQMNSLRTEDTALYYCAKDYRPRSGNHYN
NYGMDVWGPGTTVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESK
YGPPCPPCPAPEFLGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK,
and the light chain immunoglobulin comprises the amino acid sequence
(SEQ ID NO: 393)
EIVLTQSPGTLSLSPGERATLSCRASQSFRGNYLAWYQQKPGQAPRLLIYGASSRATGIP
DRFRGSGSGTDFTLTISRLEPEDFAVYYCHQYGRSPWTFGQGTKVEIKRTVAAPSVFIFPP
SDEQLKSGTASVVCLLNNFYPREAKVQWKVDNALQSGNSQESVTEQDSKDSTYSLSST
LTLSKADYEKHKVYACEVTHQGLSSPVTKSFNRGEC;
(u) the heavy chain immunoglobulin comprises the amino acid sequence
(SEQ ID NO: 411)
EVQVVESGGGLVQPGRSLRLSCTASGFTFDDYAMFWVRQGPGKGLEWVSGISWNSGSI
GYADSVKGRFTTSRDNAKNSLYLQMNSLRTEDTALYYCAKDYRPRSGNHYNNYGMD
VWGPGTTVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK,
and the light chain immunoglobulin comprises the amino acid sequence
(SEQ ID NO: 413)
LLQGSGEIVLTQSPGTLSLSPGERATLSCRASQSFRGNYLAWYQQKPGQAPRLLIYGASS
RATGIPDRFRGSGSGTDFTLTISRLEPEDFAVYYCHQYGRSPWTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC;
(v) the heavy chain immunoglobulin comprises the amino acid sequence
(SEQ ID NO: 414)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTL
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLTV
DKSRWQEGNVFSCSVMHEALHNHYTQKSLSLSLG,
and the light chain immunoglobulin comprises the amino acid sequence
(SEQ ID NO: 84)
LLQGSGDIQMTQSPSSVSASVGDRVTITCRASQGINSWLAWYQQKPGKAPKLLIYAASS
LESGVPSRFSGSGSGTDFTLTISSLQPEDFATYYCHQADSFPYTFGQGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC;
or
(c) the heavy chain immunoglobulin comprises the amino acid sequence
(SEQ ID NO: 416)
EVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCAKGLIAPRPMGFDYWGQGTL
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLTV
DKSRWQEGNVFSCSVMHEALHNHYTQKSLSLSL,
and the light chain immunoglobulin comprises the amino acid sequence
(SEQ ID NO: 84)
LLQGSGDIQMTQSPSSVSASVGDRVTITCRASQGINSWLAWYQQKPGKAPKLLIYAASS
LESGVPSRFSGSGSGTDFTLTISSLQPEDFATYYCHQADSFPYTFGQGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC.

174. The antibody-tethered drug conjugate of claim 138, comprising
(a) an immunogloubulin heavy chain comprising the amino acid sequence SEQ ID NO: 82; and
an immunogloublin light chain comprising the amino acid sequence SEQ ID NO: 84;
(b) an immunogloubulin heavy chain comprising the amino acid sequence SEQ ID NO: 414; and
an immunogloublin light chain comprising the amino acid sequence SEQ ID NO: 84;
(c) an immunogloubulin heavy chain comprising the amino acid sequence SEQ ID NO: 416; and
an immunogloublin light chain comprising the amino acid sequence SEQ ID NO: 84; or
(d) an immunogloubulin heavy chain comprising the amino acid sequence SEQ ID NO: 42; and
an immunogloublin light chain comprising the amino acid sequence SEQ ID NO: 44;
wherein a linker-payload is represented by the structure disclosed as SEQ ID NO: 507: 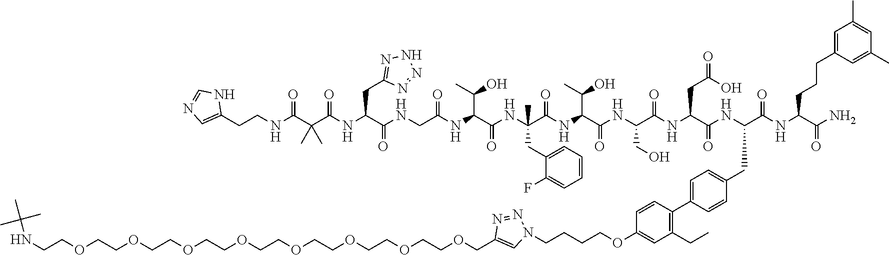
wherein] is the point of attachment of the linker-payload to amino acid 3 (Gln) of SEQ ID NO: 44 or 84,
or a pharmaceutically acceptable salt thereof.

175. A pharmaceutical composition comprising the antibody-tethered drug conjugate of claim 133, wherein at least about 80% of the antibody-tethered drug conjugate does not comprise a C-terminal lysine or lysine and glycine in any of the heavy chains.

176. The pharmaceutical composition of claim 175, wherein the heavy chain immunoglobulin that does not comprise a C-terminal lysine comprises the amino acid sequence set forth in SEQ ID NO: 414, or 416, or a variant thereof.

177. The pharmaceutical composition of claim 175, wherein less than about 20% of the antibody or antigen-binding fragment or antibody-tethered drug conjugate comprises a C-terminal lysine in at least one heavy chain.

178. The pharmaceutical composition of claim 177, wherein the at least one heavy chain that comprises a C-terminal lysine comprises the amino acid sequence set forth in SEQ ID NO: 42; 62; 82; 102; 122; 142; 162; 182; 203; 223; 243; 263; 267; 271; 291; 311; 331; 351; 371; 391; or 411; or a variant thereof.

179. A pharmaceutical composition comprising the antibody-tethered drug conjugate of claim 133 and a pharmaceutically acceptable carrier.

180. A pharmaceutical dosage form comprising the antibody-tethered drug conjugate of claim 133.

181. A vial or injection device comprising the antibody-tethered drug conjugate of claim 133.

182. A method of selectively targeting GLP1R on a surface of a cell, in the body of a subject or in vitro, with a payload, comprising administering the antibody-tethered drug conjugate of claim 133 to the subject.

183. A method of enhancing GLP1R activity, lowering blood glucose levels, lowering body weight, or treating a GLP1R-associated condition in a subject in need thereof comprising administering to the subject an effective amount of the antibody-tethered drug conjugate of claim 133.

184. A method of producing the antibody-tethered drug conjugate of claim 133 having a structure of Formula (A):
BA-(L-P)ₘ (A),
or a pharmaceutically acceptable salt thereof, the method comprising the steps of:
a) contacting, in the presence of a transglutaminase, the BA comprising at least m glutamine residues (Gln) with at least m equivalents of compound L-P, and
b) isolating the produced compound of Formula (A);
wherein BA
(i) comprises a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 26; 46; 66; 86; 106; 126; 146; 166; 187; 207; 227; 247; 275; 295; 315; 335; 355; 375; 395; 42; 62; 82; 414; 416; 102; 122; 142; 162; 182; 203; 223; 243; 263; 267; 271; 291; 311; 331; 351; 371; 391; or 411; or a variant thereof; and/or
a light chain immunoglobulin or variable region thereof that comprises CDR-L1, CDR-L2 and CDR-L3 of a light chain immunoglobulin or variable region thereof that comprises the amino acid sequence set forth in SEQ ID NO: 34; 54; 74; 94; 114; 134; 154; 174; 195; 215; 235; 255; 283; 303; 323; 343; 363; 383; 403; 44; 64; 84; 104; 124; 144; 164; 184; 205; 225; 245; 265; 269; 273; 293; 313; 333; 353; 373; 393; or 413; or a variant thereof,
(ii) is an antibody or antigen-binding fragment thereof that competes for binding to GLP1R with said antibody or antigen-binding fragment of (i);
(iii) is an antibody or antigen-binding fragment thereof that binds to the same epitope of GLP1R as said antibody or antigen-binding fragment of (i);
optionally, wherein the heavy chain immunoglobulin does not comprise a C-terminal lysine or lysine and glycine.


SEQUENCE LISTING (611 sequences)

SEQ ID NO: 1 - protein, 5 aa, synthetic construct
GGGGS

SEQ ID NO: 2 - protein, 5 aa, synthetic construct
SGGGG

SEQ ID NO: 3 - protein, 10 aa, synthetic construct
GGGGSGGGGS

SEQ ID NO: 4 - protein, 30 aa, unidentified
  SITE 30: note = Amidated Residue
HAEGTFTSDVSSYLEGQAAKEFIAWLVKGR

SEQ ID NO: 5 - protein, 8 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
XEGTFTSD

SEQ ID NO: 6 - protein, 5 aa, synthetic construct
LLQGG

SEQ ID NO: 7 - protein, 4 aa, synthetic construct
LLQG

SEQ ID NO: 8 - protein, 6 aa, synthetic construct
LSLSQG

SEQ ID NO: 9 - protein, 8 aa, synthetic construct
GGGLLQGG

SEQ ID NO: 10 - protein, 5 aa, synthetic construct
GLLQG

SEQ ID NO: 11 - protein, 10 aa, synthetic construct
GSPLAQSHGG

SEQ ID NO: 12 - protein, 7 aa, synthetic construct
GLLQGGG

SEQ ID NO: 13 - protein, 6 aa, synthetic construct
GLLQGG

SEQ ID NO: 14 - protein, 4 aa, synthetic construct
GLLQ

SEQ ID NO: 15 - protein, 8 aa, synthetic construct
LLQLLQGA

SEQ ID NO: 16 - protein, 5 aa, synthetic construct
LLQGA

SEQ ID NO: 17 - protein, 7 aa, synthetic construct
LLQYQGA

SEQ ID NO: 18 - protein, 6 aa, synthetic construct
LLQGSG

SEQ ID NO: 19 - protein, 6 aa, synthetic construct
LLQYQG

SEQ ID NO: 20 - protein, 7 aa, synthetic construct
LLQLLQG

SEQ ID NO: 21 - protein, 5 aa, synthetic construct
SLLQG

SEQ ID NO: 22 - protein, 5 aa, synthetic construct
LLQLQ

SEQ ID NO: 23 - protein, 6 aa, synthetic construct
LLQLLQ

SEQ ID NO: 24 - protein, 5 aa, synthetic construct
LLQGR

SEQ ID NO: 25 - DNA, 357 bp, synthetic construct
  source 1..357: mol_type = other DNA
gaagtgcaactggtggagtctgggggaggcctggtcaagcctggggggtccctgagactctcctgtgcagcctctggattcatcttcagtagatatagcatgaactgggtccgccaggctccagggaaggggctggagtgggtctcatccatgagtagtaatagtaaaaacacatactacgcagactcagtgaagggccgattcaccatctccagagacaacgccaaaaactcactgtttctgcaaatgaacaccctgagagccgaggacacggctgtttattactgtgcgagagatggatacaccctcagggcttttgatatctggggccaagggacaatggtcaccgtctcttca

SEQ ID NO: 26 - protein, 119 aa, synthetic construct
EVQLVESGGGLVKPGGSLRLSCAASGFIFSRYSMNWVRQAPGKGLEWVSSMSSNSKNTYYADSVKGRFTISRDNAKNSLFLQMNTLRAEDTAVYYCARDGYTLRAFDIWGQGTMVTVSS

SEQ ID NO: 27 - DNA, 24 bp, synthetic construct
  source 1..24: mol_type = other DNA
ggattcatcttcagtagatatagc

SEQ ID NO: 28 - protein, 8 aa, synthetic construct
GFIFSRYS

SEQ ID NO: 29 - DNA, 24 bp, synthetic construct
  source 1..24: mol_type = other DNA
atgagtagtaatagtaaaaacaca

SEQ ID NO: 30 - protein, 8 aa, synthetic construct
MSSNSKNT

SEQ ID NO: 31 - DNA, 36 bp, synthetic construct
  source 1..36: mol_type = other DNA
gcgagagatggatacaccctcagggcttttgatatc

SEQ ID NO: 32 - protein, 12 aa, synthetic construct
ARDGYTLRAFDI

SEQ ID NO: 33 - DNA, 324 bp, synthetic construct
  source 1..324: mol_type = other DNA
gaaattgtgttgacgcagtctccaggcaccctgtctctgtctccaggggaaagagacaccctctcctgcagggccagtcagagtattgccggcagatacgtagcctggtaccagcagaaacctggccaggcacccagactcctcatctacggtgcatccagcagggccactggcatcccagacagattcagtggcagtgggtctgggacagacttcactctcaccatcagcagactggagcctgaagattttgcagtgtattactgtcagcaatatggtagctcaccttggacgttcggccaagggaccaaggtggaaatcaaa

SEQ ID NO: 34 - protein, 114 aa, synthetic construct
LLQGSGEIVLTQSPGTLSLSPGERDTLSCRASQSIAGRYVAWYQQKPGQAPRLLIYGASSRATGIPDRFSGSGSGTDFTLTISRLEPEDFAVYYCQQYGSSPWTFGQGTKVEIK

SEQ ID NO: 35 - DNA, 21 bp, synthetic construct
  source 1..21: mol_type = other DNA
cagagtattgccggcagatac

SEQ ID NO: 36 - protein, 7 aa, synthetic construct
QSIAGRY

SEQ ID NO: 37-38 - skipped (2 entries)

SEQ ID NO: 39 - DNA, 27 bp, synthetic construct
  source 1..27: mol_type = other DNA
cagcaatatggtagctcaccttggacg

SEQ ID NO: 40 - protein, 9 aa, synthetic construct
QQYGSSPWT

SEQ ID NO: 41 - DNA, 1341 bp, synthetic construct
  source 1..1341: mol_type = other DNA
gaagtgcaactggtggagtctgggggaggcctggtcaagcctggggggtccctgagactctcctgtgcagcctctggattcatcttcagtagatatagcatgaactgggtccgccaggctccagggaaggggctggagtgggtctcatccatgagtagtaatagtaaaaacacatactacgcagactcagtgaagggccgattcaccatctccagagacaacgccaaaaactcactgtttctgcaaatgaacaccctgagagccgaggacacggctgtttattactgtgcgagagatggatacaccctcagggcttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 42 - protein, 446 aa, synthetic construct
EVQLVESGGGLVKPGGSLRLSCAASGFIFSRYSMNWVRQAPGKGLEWVSSMSSNSKNTYYADSVKGRFTISRDNAKNSLFLQMNTLRAEDTAVYYCARDGYTLRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - DNA, 666 bp, synthetic construct
  source 1..666: mol_type = other DNA
ttacttcagggatctggtgaaattgtgttgacgcagtctccaggcaccctgtctctgtctccaggggaaagagacaccctctcctgcagggccagtcagagtattgccggcagatacgtagcctggtaccagcagaaacctggccaggcacccagactcctcatctacggtgcatccagcagggccactggcatcccagacagattcagtggcagtgggtctgggacagacttcactctcaccatcagcagactggagcctgaagattttgcagtgtattactgtcagcaatatggtagctcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 44 - protein, 221 aa, synthetic construct
LLQGSGEIVLTQSPGTLSLSPGERDTLSCRASQSIAGRYVAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 363 bp, synthetic construct
  source 1..363: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctca

SEQ ID NO: 46 - protein, 121 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSS

SEQ ID NO: 47 - DNA, 24 bp, synthetic construct
  source 1..24: mol_type = other DNA
ggattcacctttagcagctatgcc

SEQ ID NO: 48 - protein, 8 aa, synthetic construct
GFTFSSYA

SEQ ID NO: 49 - DNA, 24 bp, synthetic construct
  source 1..24: mol_type = other DNA
attagcggtagtggtggcagcaca

SEQ ID NO: 50 - protein, 8 aa, synthetic construct
ISGSGGST

SEQ ID NO: 51 - DNA, 42 bp, synthetic construct
  source 1..42: mol_type = other DNA
gcgaaaggccttatagcacctcgtccgatgggctttgactac

SEQ ID NO: 52 - protein, 14 aa, synthetic construct
AKGLIAPRPMGFDY

SEQ ID NO: 53 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaa

SEQ ID NO: 54 - protein, 107 aa, synthetic construct
DIQMTQSPSSVSASVGDRVTITCRASQGINSWLAWYQQKPGKAPKLLIYAASSLQSGVPSRFSGSGSGTDFTLTISSLQPEDFATYYCHQADSFPYTFGQGTKLEIK

SEQ ID NO: 55 - DNA, 18 bp, synthetic construct
  source 1..18: mol_type = other DNA
cagggtattaacagctgg

SEQ ID NO: 56 - protein, 6 aa, synthetic construct
QGINSW

SEQ ID NO: 57-58 - skipped (2 entries)

SEQ ID NO: 59 - DNA, 27 bp, synthetic construct
  source 1..27: mol_type = other DNA
caccaggctgacagtttcccgtacact

SEQ ID NO: 60 - protein, 9 aa, synthetic construct
HQADSFPYT

SEQ ID NO: 61 - DNA, 1347 bp, synthetic construct
  source 1..1347: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 62 - protein, 448 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - DNA, 663 bp, synthetic construct
  source 1..663: mol_type = other DNA
ctgctgcaaggctctggcgacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 64 - protein, 220 aa, synthetic construct
LLQGSGDIQMTQSPSSVSASVGDRVTITCRASQGINSWLAWYQQKPGKAPKLLIYAASSLQSGVPSRFSGSGSGTDFTLTISSLQPEDFATYYCHQADSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 363 bp, synthetic construct; identical to SEQ ID NO: 45
  source 1..363: mol_type = other DNA

SEQ ID NO: 66 - protein, 121 aa, synthetic construct; identical to SEQ ID NO: 46

SEQ ID NO: 67 - DNA, 24 bp, synthetic construct
  source 1..24: mol_type = other DNA
ggattcacctttagcagctatgcc

SEQ ID NO: 68 - protein, 8 aa, synthetic construct
GFTFSSYA

SEQ ID NO: 69 - DNA, 24 bp, synthetic construct
  source 1..24: mol_type = other DNA
attagcggtagtggtggcagcaca

SEQ ID NO: 70 - protein, 8 aa, synthetic construct
ISGSGGST

SEQ ID NO: 71 - DNA, 42 bp, synthetic construct; identical to SEQ ID NO: 51
  source 1..42: mol_type = other DNA

SEQ ID NO: 72 - protein, 14 aa, synthetic construct
AKGLIAPRPMGFDY

SEQ ID NO: 73 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttggaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaa

SEQ ID NO: 74 - protein, 107 aa, synthetic construct
DIQMTQSPSSVSASVGDRVTITCRASQGINSWLAWYQQKPGKAPKLLIYAASSLESGVPSRFSGSGSGTDFTLTISSLQPEDFATYYCHQADSFPYTFGQGTKLEIK

SEQ ID NO: 75 - DNA, 18 bp, synthetic construct
  source 1..18: mol_type = other DNA
cagggtattaacagctgg

SEQ ID NO: 76 - protein, 6 aa, synthetic construct
QGINSW

SEQ ID NO: 77-78 - skipped (2 entries)

SEQ ID NO: 79 - DNA, 27 bp, synthetic construct
  source 1..27: mol_type = other DNA
caccaggctgacagtttcccgtacact

SEQ ID NO: 80 - protein, 9 aa, synthetic construct
HQADSFPYT

SEQ ID NO: 81 - DNA, 1347 bp, synthetic construct; identical to SEQ ID NO: 61
  source 1..1347: mol_type = other DNA

SEQ ID NO: 82 - protein, 448 aa, synthetic construct; identical to SEQ ID NO: 62

SEQ ID NO: 83 - DNA, 663 bp, synthetic construct
  source 1..663: mol_type = other DNA
ctgctgcaaggctctggcgacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttggaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 84 - protein, 220 aa, synthetic construct
LLQGSGDIQMTQSPSSVSASVGDRVTITCRASQGINSWLAWYQQKPGKAPKLLIYAASSLESGVPSRFSGSGSGTDFTLTISSLQPEDFATYYCHQADSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - DNA, 348 bp, synthetic construct
  source 1..348: mol_type = other DNA
caggtgcagctggtggagtctgggggaggcgtgggccagcctgggaggtccctgagactgtcctgtgcagcctctggattcaccttcagcaggaatgccatgcactgggtccgccaggctccaggcaaggggctggagtgggtggcagtcatatcatatgatggaagtaataaacactatgcagactccgtgaagggccgattcaccatctccagagacaattccaagaacacgctgtatctggaaatgaacagcctgagagttgaggacacggctgtgtattattgtgcgaaaggggggattccttttgactactggggccagggaaccctggtcaccgtctcctca

SEQ ID NO: 86 - protein, 116 aa, synthetic construct
QVQLVESGGGVGQPGRSLRLSCAASGFTFSRNAMHWVRQAPGKGLEWVAVISYDGSNKHYADSVKGRFTISRDNSKNTLYLEMNSLRVEDTAVYYCAKGGIPFDYWGQGTLVTVSS

SEQ ID NO: 87 - DNA, 24 bp, synthetic construct
  source 1..24: mol_type = other DNA
ggattcaccttcagcaggaatgcc

SEQ ID NO: 88 - protein, 8 aa, synthetic construct
GFTFSRNA

SEQ ID NO: 89 - DNA, 24 bp, synthetic construct
  source 1..24: mol_type = other DNA
atatcatatgatggaagtaataaa

SEQ ID NO: 90 - protein, 8 aa, synthetic construct
ISYDGSNK

SEQ ID NO: 91 - DNA, 27 bp, synthetic construct
  source 1..27: mol_type = other DNA
gcgaaaggggggattccttttgactac

SEQ ID NO: 92 - protein, 9 aa, synthetic construct
AKGGIPFDY

SEQ ID NO: 93 - DNA, 336 bp, synthetic construct
  source 1..336: mol_type = other DNA
gatattgtgatgacccagtctccactctcctcacctgtcacccttggacagccggcctccatctcctgcaggtctagtcaaagcctcgtacactttgatggaaacacctacttgagttggcttcaccagaggccaggccagcctccaagactcctaatttataagatttctaaccgcttctctggggtcccagacagattcagtggcagtggggcagggacagatttcacactgaaaatcagcagggtggaacctgaagatgtcggggtttattactgcatgcatgctacacaatttccgtacacttttggccaggggaccaagctggagatcaaa

SEQ ID NO: 94 - protein, 112 aa, synthetic construct
DIVMTQSPLSSPVTLGQPASISCRSSQSLVHFDGNTYLSWLHQRPGQPPRLLIYKISNRFSGVPDRFSGSGAGTDFTLKISRVEPEDVGVYYCMHATQFPYTFGQGTKLEIK

SEQ ID NO: 95 - DNA, 33 bp, synthetic construct
  source 1..33: mol_type = other DNA
caaagcctcgtacactttgatggaaacacctac

SEQ ID NO: 96 - protein, 11 aa, synthetic construct
QSLVHFDGNTY

SEQ ID NO: 97-98 - skipped (2 entries)

SEQ ID NO: 99 - DNA, 27 bp, synthetic construct
  source 1..27: mol_type = other DNA
atgcatgctacacaatttccgtacact

SEQ ID NO: 100 - protein, 9 aa, synthetic construct
MHATQFPYT

SEQ ID NO: 101 - DNA, 1332 bp, synthetic construct
  source 1..1332: mol_type = other DNA
caggtgcagctggtggagtctgggggaggcgtgggccagcctgggaggtccctgagactgtcctgtgcagcctctggattcaccttcagcaggaatgccatgcactgggtccgccaggctccaggcaaggggctggagtgggtggcagtcatatcatatgatggaagtaataaacactatgcagactccgtgaagggccgattcaccatctccagagacaattccaagaacacgctgtatctggaaatgaacagcctgagagttgaggacacggctgtgtattattgtgcgaaaggggggattccttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 102 - protein, 443 aa, synthetic construct
QVQLVESGGGVGQPGRSLRLSCAASGFTFSRNAMHWVRQAPGKGLEWVAVISYDGSNKHYADSVKGRFTISRDNSKNTLYLEMNSLRVEDTAVYYCAKGGIPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - DNA, 678 bp, synthetic construct
  source 1..678: mol_type = other DNA
ctgctgcaaggctctggcgatattgtgatgacccagtctccactctcctcacctgtcacccttggacagccggcctccatctcctgcaggtctagtcaaagcctcgtacactttgatggaaacacctacttgagttggcttcaccagaggccaggccagcctccaagactcctaatttataagatttctaaccgcttctctggggtcccagacagattcagtggcagtggggcagggacagatttcacactgaaaatcagcagggtggaacctgaagatgtcggggtttattactgcatgcatgctacacaatttccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 104 - protein, 225 aa, synthetic construct
LLQGSGDIVMTQSPLSSPVTLGQPASISCRSSQSLVHFDGNTYLSWLHQRPGQPPRLLIYKISNRFSGVPDRFSGSGAGTDFTLKISRVEPEDVGVYYCMHATQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 357 bp, synthetic construct
  source 1..357: mol_type = other DNA
caggtgcagctggtggagtctgggggaggcgtggtccagcctgcgaggtccctgagactctcctgtgcagcctctggattcgccttcagtaggtctgccatgcactgggtccgccaggctccaggcaaggggctggagtgggtggcagttatatcatatgatggaagtaataaatactatacagactccgtgaagggccgattcaccatctccagagacaattccaagaacacgctgtatctgcaaatgaacaccctgagagctgaggacacggctctttattactgtgcgaaaatgtatacaactatggactcttttgactactggggccagggaaccctggtcaccgtctcctca

SEQ ID NO: 106 - protein, 119 aa, synthetic construct
QVQLVESGGGVVQPARSLRLSCAASGFAFSRSAMHWVRQAPGKGLEWVAVISYDGSNKYYTDSVKGRFTISRDNSKNTLYLQMNTLRAEDTALYYCAKMYTTMDSFDYWGQGTLVTVSS

SEQ ID NO: 107 - DNA, 24 bp, synthetic construct
  source 1..24: mol_type = other DNA
ggattcgccttcagtaggtctgcc

SEQ ID NO: 108 - protein, 8 aa, synthetic construct
GFAFSRSA

SEQ ID NO: 109 - DNA, 24 bp, synthetic construct
  source 1..24: mol_type = other DNA
atatcatatgatggaagtaataaa

SEQ ID NO: 110 - protein, 8 aa, synthetic construct
ISYDGSNK

SEQ ID NO: 111 - DNA, 36 bp, synthetic construct
  source 1..36: mol_type = other DNA
gcgaaaatgtatacaactatggactcttttgactac

SEQ ID NO: 112 - protein, 12 aa, synthetic construct
AKMYTTMDSFDY

SEQ ID NO: 113 - DNA, 321 bp, synthetic construct
  source 1..321: mol_type = other DNA
gacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcactttattactgtcaacagcttaatagttaccctcggacgttcggccaagggaccaaggtggaaatcaaa

SEQ ID NO: 114 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFALYYCQQLNSYPRTFGQGTKVEIK

SEQ ID NO: 115 - DNA, 18 bp, synthetic construct
  source 1..18: mol_type = other DNA
cagggcattagcagttat

SEQ ID NO: 116 - protein, 6 aa, synthetic construct
QGISSY

SEQ ID NO: 117-118 - skipped (2 entries)

SEQ ID NO: 119 - DNA, 27 bp, synthetic construct
  source 1..27: mol_type = other DNA
caacagcttaatagttaccctcggacg

SEQ ID NO: 120 - protein, 9 aa, synthetic construct
QQLNSYPRT

SEQ ID NO: 121 - DNA, 1341 bp, synthetic construct
  source 1..1341: mol_type = other DNA
caggtgcagctggtggagtctgggggaggcgtggtccagcctgcgaggtccctgagactctcctgtgcagcctctggattcgccttcagtaggtctgccatgcactgggtccgccaggctccaggcaaggggctggagtgggtggcagttatatcatatgatggaagtaataaatactatacagactccgtgaagggccgattcaccatctccagagacaattccaagaacacgctgtatctgcaaatgaacaccctgagagctgaggacacggctctttattactgtgcgaaaatgtatacaactatggactcttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 122 - protein, 446 aa, synthetic construct
QVQLVESGGGVVQPARSLRLSCAASGFAFSRSAMHWVRQAPGKGLEWVAVISYDGSNKYYTDSVKGRFTISRDNSKNTLYLQMNTLRAEDTALYYCAKMYTTMDSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - DNA, 663 bp, synthetic construct
  source 1..663: mol_type = other DNA
ctgctgcaaggctctggcgacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcactttattactgtcaacagcttaatagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 124 - protein, 220 aa, synthetic construct
LLQGSGDIQLTQSPSFLSASVGDRVTITCWASQGISSYLAWYQQKPGKAPKLLIYAASTLQSGVPSRFSGSGSGTEFTLTISSLQPEDFALYYCQQLNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - DNA, 357 bp, synthetic construct; identical to SEQ ID NO: 25
  source 1..357: mol_type = other DNA

SEQ ID NO: 126 - protein, 119 aa, synthetic construct; identical to SEQ ID NO: 26

SEQ ID NO: 127 - DNA, 24 bp, synthetic construct
  source 1..24: mol_type = other DNA
ggattcatcttcagtagatatagc

SEQ ID NO: 128 - protein, 8 aa, synthetic construct
GFIFSRYS

SEQ ID NO: 129 - DNA, 24 bp, synthetic construct
  source 1..24: mol_type = other DNA
atgagtagtaatagtaaaaacaca

SEQ ID NO: 130 - protein, 8 aa, synthetic construct
MSSNSKNT

SEQ ID NO: 131 - DNA, 36 bp, synthetic construct; identical to SEQ ID NO: 31
  source 1..36: mol_type = other DNA

SEQ ID NO: 132 - protein, 12 aa, synthetic construct
ARDGYTLRAFDI

SEQ ID NO: 133 - DNA, 324 bp, synthetic construct; identical to SEQ ID NO: 33
  source 1..324: mol_type = other DNA

SEQ ID NO: 134 - protein, 108 aa, synthetic construct
EIVLTQSPGTLSLSPGERDTLSCRASQSIAGRYVAWYQQKPGQAPRLLIYGASSRATGIPDRFSGSGSGTDFTLTISRLEPEDFAVYYCQQYGSSPWTFGQGTKVEIK

SEQ ID NO: 135 - DNA, 21 bp, synthetic construct
  source 1..21: mol_type = other DNA
cagagtattgccggcagatac

SEQ ID NO: 136 - protein, 7 aa, synthetic construct
QSIAGRY

SEQ ID NO: 137-138 - skipped (2 entries)

SEQ ID NO: 139 - DNA, 27 bp, synthetic construct
  source 1..27: mol_type = other DNA
cagcaatatggtagctcaccttggacg

SEQ ID NO: 140 - protein, 9 aa, synthetic construct
QQYGSSPWT

SEQ ID NO: 141 - DNA, 1359 bp, synthetic construct
  source 1..1359: mol_type = other DNA
ttacttcagggatctggtgaagtgcaactggtggagtctgggggaggcctggtcaagcctggggggtccctgagactctcctgtgcagcctctggattcatcttcagtagatatagcatgaactgggtccgccaggctccagggaaggggctggagtgggtctcatccatgagtagtaatagtaaaaacacatactacgcagactcagtgaagggccgattcaccatctccagagacaacgccaaaaactcactgtttctgcaaatgaacaccctgagagccgaggacacggctgtttattactgtgcgagagatggatacaccctcagggcttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 142 - protein, 452 aa, synthetic construct
LLQGSGEVQLVESGGGLVKPGGSLRLSCAASGFIFSRYSMNWVRQAPGKGLEWVSSMSSNSKNTYYADSVKGRFTISRDNAKNSLFLQMNTLRAEDTAVYYCARDGYTLRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - DNA, 648 bp, synthetic construct
  source 1..648: mol_type = other DNA
gaaattgtgttgacgcagtctccaggcaccctgtctctgtctccaggggaaagagacaccctctcctgcagggccagtcagagtattgccggcagatacgtagcctggtaccagcagaaacctggccaggcacccagactcctcatctacggtgcatccagcagggccactggcatcccagacagattcagtggcagtgggtctgggacagacttcactctcaccatcagcagactggagcctgaagattttgcagtgtattactgtcagcaatatggtagctcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 144 - protein, 215 aa, synthetic construct
EIVLTQSPGTLSLSPGERDTLSCRASQSIAGRYVAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - DNA, 375 bp, synthetic construct
  source 1..375: mol_type = other DNA
caggtgcagctggtggagtctgggggaggcgtggtccagcctgggaggtccctgagactctcctgtgcagcgtctggattcaccttcagtggctatggcatacactgggtccgccaggctccaggcaagggactggtgtgggtggcagttatatggtatgatggaagttttaaatactatgcagactccgtgaagggccgattcaccatctccagagacaattccaagaacacgctgtatctgcagatgaacagcctgagagccgaggacacggctgtgtattactgtgcgagaggtgatagcagctcgtccggacggtactactactacggtatggacgtctggggccaagggaccacggtcaccgtctcctca

SEQ ID NO: 146 - protein, 125 aa, synthetic construct
QVQLVESGGGVVQPGRSLRLSCAASGFTFSGYGIHWVRQAPGKGLVWVAVIWYDGSFKYYADSVKGRFTISRDNSKNTLYLQMNSLRAEDTAVYYCARGDSSSSGRYYYYGMDVWGQGTTVTVSS

SEQ ID NO: 147 - DNA, 24 bp, synthetic construct
  source 1..24: mol_type = other DNA
ggattcaccttcagtggctatggc

SEQ ID NO: 148 - protein, 8 aa, synthetic construct
GFTFSGYG

SEQ ID NO: 149 - DNA, 24 bp, synthetic construct
  source 1..24: mol_type = other DNA
atatggtatgatggaagttttaaa

SEQ ID NO: 150 - protein, 8 aa, synthetic construct
IWYDGSFK

SEQ ID NO: 151 - DNA, 54 bp, synthetic construct
  source 1..54: mol_type = other DNA
gcgagaggtgatagcagctcgtccggacggtactactactacggtatggacgtc

SEQ ID NO: 152 - protein, 18 aa, synthetic construct
ARGDSSSSGRYYYYGMDV

SEQ ID NO: 153 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggacagcccctaagcgcctgatctttgctgcatccagtttgcaaagtggggtcccatcaaggttcagcggcagtggatctgggacagaattcactctcacaatcagcagcctgcagcctgaagattttgcgacttattactgtctacagcataataattaccctcccactttcggcggagggaccaaggtggagatcaaa

SEQ ID NO: 154 - protein, 107 aa, synthetic construct
DIQMTQSPSSLSASVGDRVTITCRASQGIRNDLGWYQQKPGTAPKRLIFAASSLQSGVPSRFSGSGSGTEFTLTISSLQPEDFATYYCLQHNNYPPTFGGGTKVEIK

SEQ ID NO: 155 - DNA, 18 bp, synthetic construct
  source 1..18: mol_type = other DNA
cagggcattagaaatgat

SEQ ID NO: 156 - protein, 6 aa, synthetic construct
QGIRND

SEQ ID NO: 157-158 - skipped (2 entries)

SEQ ID NO: 159 - DNA, 27 bp, synthetic construct
  source 1..27: mol_type = other DNA
ctacagcataataattaccctcccact

SEQ ID NO: 160 - protein, 9 aa, synthetic construct
LQHNNYPPT

SEQ ID NO: 161 - DNA, 1359 bp, synthetic construct
  source 1..1359: mol_type = other DNA
caggtgcagctggtggagtctgggggaggcgtggtccagcctgggaggtccctgagactctcctgtgcagcgtctggattcaccttcagtggctatggcatacactgggtccgccaggctccaggcaagggactggtgtgggtggcagttatatggtatgatggaagttttaaatactatgcagactccgtgaagggccgattcaccatctccagagacaattccaagaacacgctgtatctgcagatgaacagcctgagagccgaggacacggctgtgtattactgtgcgagaggtgatagcagctcgtccggacggtactactactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 162 - protein, 452 aa, synthetic construct
QVQLVESGGGVVQPGRSLRLSCAASGFTFSGYGIHWVRQAPGKGLVWVAVIWYDGSFKYYADSVKGRFTISRDNSKNTLYLQMNSLRAEDTAVYYCARGDSSSSGRYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - DNA, 663 bp, synthetic construct
  source 1..663: mol_type = other DNA
ctgctgcaaggctctggcgacatccagatgacccagtctccatcctccctgtctgcatctgtaggagacagagtcaccatcacttgccgggcaagtcagggcattagaaatgatttaggctggtatcagcagaaaccagggacagcccctaagcgcctgatctttgctgcatccagtttgcaaagtggggtcccatcaaggttcagcggcagtggatctgggacagaattcactctcacaatcagcagcctgcagcctgaagattttgcgacttattactgtctacagcataataattaccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 164 - protein, 220 aa, synthetic construct
LLQGSGDIQMTQSPSSLSASVGDRVTITCRASQGIRNDLGWYQQKPGTAPKRLIFAASSLQSGVPSRFSGSGSGTEFTLTISSLQPEDFATYYCLQHNNYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - DNA, 366 bp, synthetic construct
  source 1..366: mol_type = other DNA
gaggtgcagctggtggagtctgggggaggcttggtccagcctggggggtccctgaaactctcctgtgcagcctctgggttcaccttcagtggctctgctatgcactgggtccgccaggcttccgggaaagggctggagtgggttggccgtattacaagcaaagctaacagttacgcgacagcatatgatgcgtcggtgaaaggcaggttcaccatctccagagatgattcaaagaacacggcgtatctgcaaatgaacagcctgaaaaccgaggacacggccgtgtattactgtactaggcaacgatttttggagtttttattccttgactactggggccagggaaccctggtcaccgtctcctca

SEQ ID NO: 166 - protein, 122 aa, synthetic construct
EVQLVESGGGLVQPGGSLKLSCAASGFTFSGSAMHWVRQASGKGLEWVGRITSKANSYATAYDASVKGRFTISRDDSKNTAYLQMNSLKTEDTAVYYCTRQRFLEFLFLDYWGQGTLVTVSS

SEQ ID NO: 167 - DNA, 24 bp, synthetic construct
  source 1..24: mol_type = other DNA
gggttcaccttcagtggctctgct

SEQ ID NO: 168 - protein, 8 aa, synthetic construct
GFTFSGSA

SEQ ID NO: 169 - DNA, 30 bp, synthetic construct
  source 1..30: mol_type = other DNA
attacaagcaaagctaacagttacgcgaca

SEQ ID NO: 170 - protein, 10 aa, synthetic construct
ITSKANSYAT

SEQ ID NO: 171 - DNA, 39 bp, synthetic construct
  source 1..39: mol_type = other DNA
actaggcaacgatttttggagtttttattccttgactac

SEQ ID NO: 172 - protein, 13 aa, synthetic construct
TRQRFLEFLFLDY

SEQ ID NO: 173 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 174 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 175 - DNA, 18 bp, synthetic construct
  source 1..18: mol_type = other DNA
cagagcattagcagctat

SEQ ID NO: 176 - protein, 6 aa, synthetic construct
QSISSY

SEQ ID NO: 177-178 - skipped (2 entries)

SEQ ID NO: 179 - DNA, 30 bp, synthetic construct
  source 1..30: mol_type = other DNA
caacagagttacagtacccctccgatcacc

SEQ ID NO: 180 - protein, 10 aa, synthetic construct
QQSYSTPPIT

SEQ ID NO: 181 - DNA, 1350 bp, synthetic construct
  source 1..1350: mol_type = other DNA
gaggtgcagctggtggagtctgggggaggcttggtccagcctggggggtccctgaaactctcctgtgcagcctctgggttcaccttcagtggctctgctatgcactgggtccgccaggcttccgggaaagggctggagtgggttggccgtattacaagcaaagctaacagttacgcgacagcatatgatgcgtcggtgaaaggcaggttcaccatctccagagatgattcaaagaacacggcgtatctgcaaatgaacagcctgaaaaccgaggacacggccgtgtattactgtactaggcaacgatttttggagtttttattccttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 182 - protein, 449 aa, synthetic construct
EVQLVESGGGLVQPGGSLKLSCAASGFTFSGSAMHWVRQASGKGLEWVGRITSKANSYATAYDASVKGRFTISRDDSKNTAYLQMNSLKTEDTAVYYCTRQRFLEFLFLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 183 - DNA, 666 bp, synthetic construct
  source 1..666: mol_type = other DNA
ctgctgcaaggctctggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 184 - protein, 221 aa, synthetic construct
LLQGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 8 aa, synthetic construct
LLQGSGSG

SEQ ID NO: 186 - DNA, 363 bp, synthetic construct; identical to SEQ ID NO: 45
  source 1..363: mol_type = other DNA

SEQ ID NO: 187 - protein, 121 aa, synthetic construct; identical to SEQ ID NO: 46

SEQ ID NO: 188 - DNA, 24 bp, synthetic construct
  source 1..24: mol_type = other DNA
ggattcacctttagcagctatgcc

SEQ ID NO: 189 - protein, 8 aa, synthetic construct
GFTFSSYA

SEQ ID NO: 190 - DNA, 24 bp, synthetic construct
  source 1..24: mol_type = other DNA
attagcggtagtggtggcagcaca

SEQ ID NO: 191 - protein, 8 aa, synthetic construct
ISGSGGST

SEQ ID NO: 192 - DNA, 42 bp, synthetic construct; identical to SEQ ID NO: 51
  source 1..42: mol_type = other DNA

SEQ ID NO: 193 - protein, 14 aa, synthetic construct
AKGLIAPRPMGFDY

SEQ ID NO: 194 - DNA, 321 bp, synthetic construct; identical to SEQ ID NO: 53
  source 1..321: mol_type = other DNA

SEQ ID NO: 195 - protein, 107 aa, synthetic construct; identical to SEQ ID NO: 54

SEQ ID NO: 196 - DNA, 18 bp, synthetic construct
  source 1..18: mol_type = other DNA
cagggtattaacagctgg

SEQ ID NO: 197 - protein, 6 aa, synthetic construct
QGINSW

SEQ ID NO: 198-199 - skipped (2 entries)

SEQ ID NO: 200 - DNA, 27 bp, synthetic construct
  source 1..27: mol_type = other DNA
caccaggctgacagtttcccgtacact

SEQ ID NO: 201 - protein, 9 aa, synthetic construct
HQADSFPYT

SEQ ID NO: 202 - DNA, 1365 bp, synthetic construct
  source 1..1365: mol_type = other DNA
ctgctgcaaggctctggcgaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 203 - protein, 454 aa, synthetic construct
LLQGSGEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - DNA, 645 bp, synthetic construct
  source 1..645: mol_type = other DNA
gacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 205 - protein, 214 aa, synthetic construct
DIQMTQSPSSVSASVGDRVTITCRASQGINSWLAWYQQKPGKAPKLLIYAASSLQSGVPSRFSGSGSGTDFTLTISSLQPEDFATYYCHQADSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - DNA, 348 bp, synthetic construct; identical to SEQ ID NO: 85
  source 1..348: mol_type = other DNA

SEQ ID NO: 207 - protein, 115 aa, synthetic construct
VQLVESGGGVGQPGRSLRLSCAASGFTFSRNAMHWVRQAPGKGLEWVAVISYDGSNKHYADSVKGRFTISRDNSKNTLYLEMNSLRVEDTAVYYCAKGGIPFDYWGQGTLVTVSS

SEQ ID NO: 208 - DNA, 24 bp, synthetic construct
  source 1..24: mol_type = other DNA
ggattcaccttcagcaggaatgcc

SEQ ID NO: 209 - protein, 8 aa, synthetic construct
GFTFSRNA

SEQ ID NO: 210 - DNA, 24 bp, synthetic construct
  source 1..24: mol_type = other DNA
atatcatatgatggaagtaataaa

SEQ ID NO: 211 - protein, 8 aa, synthetic construct
ISYDGSNK

SEQ ID NO: 212 - DNA, 27 bp, synthetic construct
  source 1..27: mol_type = other DNA
gcgaaaggggggattccttttgactac

SEQ ID NO: 213 - protein, 9 aa, synthetic construct
AKGGIPFDY

SEQ ID NO: 214 - DNA, 336 bp, synthetic construct; identical to SEQ ID NO: 93
  source 1..336: mol_type = other DNA

SEQ ID NO: 215 - protein, 112 aa, synthetic construct; identical to SEQ ID NO: 94

SEQ ID NO: 216 - DNA, 33 bp, synthetic construct; identical to SEQ ID NO: 95
  source 1..33: mol_type = other DNA

SEQ ID NO: 217 - protein, 11 aa, synthetic construct
QSLVHFDGNTY

SEQ ID NO: 218-219 - skipped (2 entries)

SEQ ID NO: 220 - DNA, 27 bp, synthetic construct
  source 1..27: mol_type = other DNA
atgcatgctacacaatttccgtacact

SEQ ID NO: 221 - protein, 9 aa, synthetic construct
MHATQFPYT

SEQ ID NO: 222 - DNA, 1350 bp, synthetic construct
  source 1..1350: mol_type = other DNA
ctgctgcaaggctctggccaggtgcagctggtggagtctgggggaggcgtgggccagcctgggaggtccctgagactgtcctgtgcagcctctggattcaccttcagcaggaatgccatgcactgggtccgccaggctccaggcaaggggctggagtgggtggcagtcatatcatatgatggaagtaataaacactatgcagactccgtgaagggccgattcaccatctccagagacaattccaagaacacgctgtatctggaaatgaacagcctgagagttgaggacacggctgtgtattattgtgcgaaaggggggattccttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 223 - protein, 449 aa, synthetic construct
LLQGSGQVQLVESGGGVGQPGRSLRLSCAASGFTFSRNAMHWVRQAPGKGLEWVAVISYDGSNKHYADSVKGRFTISRDNSKNTLYLEMNSLRVEDTAVYYCAKGGIPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 224 - DNA, 660 bp, synthetic construct
  source 1..660: mol_type = other DNA
gatattgtgatgacccagtctccactctcctcacctgtcacccttggacagccggcctccatctcctgcaggtctagtcaaagcctcgtacactttgatggaaacacctacttgagttggcttcaccagaggccaggccagcctccaagactcctaatttataagatttctaaccgcttctctggggtcccagacagattcagtggcagtggggcagggacagatttcacactgaaaatcagcagggtggaacctgaagatgtcggggtttattactgcatgcatgctacacaatttccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 225 - protein, 219 aa, synthetic construct
DIVMTQSPLSSPVTLGQPASISCRSSQSLVHFDGNTYLSWLHQRPGQPPRLLIYKISNRFSGVPDRFSGSGAGTDFTLKISRVEPEDVGVYYCMHATQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - DNA, 357 bp, synthetic construct; identical to SEQ ID NO: 105
  source 1..357: mol_type = other DNA

SEQ ID NO: 227 - protein, 119 aa, synthetic construct; identical to SEQ ID NO: 106

SEQ ID NO: 228 - DNA, 24 bp, synthetic construct
  source 1..24: mol_type = other DNA
ggattcgccttcagtaggtctgcc

SEQ ID NO: 229 - protein, 8 aa, synthetic construct
GFAFSRSA

SEQ ID NO: 230 - DNA, 24 bp, synthetic construct
  source 1..24: mol_type = other DNA
atatcatatgatggaagtaataaa

SEQ ID NO: 231 - protein, 8 aa, synthetic construct
ISYDGSNK

SEQ ID NO: 232 - DNA, 36 bp, synthetic construct; identical to SEQ ID NO: 111
  source 1..36: mol_type = other DNA

SEQ ID NO: 233 - protein, 12 aa, synthetic construct
AKMYTTMDSFDY

SEQ ID NO: 234 - DNA, 321 bp, synthetic construct; identical to SEQ ID NO: 113
  source 1..321: mol_type = other DNA

SEQ ID NO: 235 - protein, 107 aa, synthetic construct; identical to SEQ ID NO: 114

SEQ ID NO: 236 - DNA, 18 bp, synthetic construct
  source 1..18: mol_type = other DNA
cagggcattagcagttat

SEQ ID NO: 237 - protein, 6 aa, synthetic construct
QGISSY

SEQ ID NO: 238-239 - skipped (2 entries)

SEQ ID NO: 240 - DNA, 27 bp, synthetic construct
  source 1..27: mol_type = other DNA
caacagcttaatagttaccctcggacg

SEQ ID NO: 241 - protein, 9 aa, synthetic construct
QQLNSYPRT

SEQ ID NO: 242 - DNA, 1359 bp, synthetic construct
  source 1..1359: mol_type = other DNA
ctgctgcaaggctctggccaggtgcagctggtggagtctgggggaggcgtggtccagcctgcgaggtccctgagactctcctgtgcagcctctggattcgccttcagtaggtctgccatgcactgggtccgccaggctccaggcaaggggctggagtgggtggcagttatatcatatgatggaagtaataaatactatacagactccgtgaagggccgattcaccatctccagagacaattccaagaacacgctgtatctgcaaatgaacaccctgagagctgaggacacggctctttattactgtgcgaaaatgtatacaactatggactcttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 243 - protein, 452 aa, synthetic construct
LLQGSGQVQLVESGGGVVQPARSLRLSCAASGFAFSRSAMHWVRQAPGKGLEWVAVISYDGSNKYYTDSVKGRFTISRDNSKNTLYLQMNTLRAEDTALYYCAKMYTTMDSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 244 - DNA, 645 bp, synthetic construct
  source 1..645: mol_type = other DNA
gacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcactttattactgtcaacagcttaatagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 245 - protein, 214 aa, synthetic construct
DIQLTQSPSFLSASVGDRVTITCWASQGISSYLAWYQQKPGKAPKLLIYAASTLQSGVPSRFSGSGSGTEFTLTISSLQPEDFALYYCQQLNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - DNA, 366 bp, synthetic construct; identical to SEQ ID NO: 165
  source 1..366: mol_type = other DNA

SEQ ID NO: 247 - protein, 122 aa, synthetic construct; identical to SEQ ID NO: 166

SEQ ID NO: 248 - DNA, 24 bp, synthetic construct
  source 1..24: mol_type = other DNA
gggttcaccttcagtggctctgct

SEQ ID NO: 249 - protein, 8 aa, synthetic construct
GFTFSGSA

SEQ ID NO: 250 - DNA, 30 bp, synthetic construct; identical to SEQ ID NO: 169
  source 1..30: mol_type = other DNA

SEQ ID NO: 251 - protein, 10 aa, synthetic construct
ITSKANSYAT

SEQ ID NO: 252 - DNA, 39 bp, synthetic construct; identical to SEQ ID NO: 171
  source 1..39: mol_type = other DNA

SEQ ID NO: 253 - protein, 13 aa, synthetic construct
TRQRFLEFLFLDY

SEQ ID NO: 254 - DNA, 324 bp, synthetic construct; identical to SEQ ID NO: 173
  source 1..324: mol_type = other DNA

SEQ ID NO: 255 - protein, 108 aa, synthetic construct; identical to SEQ ID NO: 174

SEQ ID NO: 256 - DNA, 18 bp, synthetic construct
  source 1..18: mol_type = other DNA
cagagcattagcagctat

SEQ ID NO: 257 - protein, 6 aa, synthetic construct
QSISSY

SEQ ID NO: 258-259 - skipped (2 entries)

SEQ ID NO: 260 - DNA, 30 bp, synthetic construct; identical to SEQ ID NO: 179
  source 1..30: mol_type = other DNA

SEQ ID NO: 261 - protein, 10 aa, synthetic construct
QQSYSTPPIT

SEQ ID NO: 262 - DNA, 1368 bp, synthetic construct
  source 1..1368: mol_type = other DNA
ctgctgcaaggctctggcgaggtgcagctggtggagtctgggggaggcttggtccagcctggggggtccctgaaactctcctgtgcagcctctgggttcaccttcagtggctctgctatgcactgggtccgccaggcttccgggaaagggctggagtgggttggccgtattacaagcaaagctaacagttacgcgacagcatatgatgcgtcggtgaaaggcaggttcaccatctccagagatgattcaaagaacacggcgtatctgcaaatgaacagcctgaaaaccgaggacacggccgtgtattactgtactaggcaacgatttttggagtttttattccttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 263 - protein, 455 aa, synthetic construct
LLQGSGEVQLVESGGGLVQPGGSLKLSCAASGFTFSGSAMHWVRQASGKGLEWVGRITSKANSYATAYDASVKGRFTISRDDSKNTAYLQMNSLKTEDTAVYYCTRQRFLEFLFLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 264 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 265 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - DNA, 1362 bp, synthetic construct
  source 1..1362: mol_type = other DNA
ttacttcagggatctggtcaagttactcttaaagaatctggtcctggaattcttcaaccttctcaaactctttctcttacttgttctttttctggtttttctctttctacttctggtactggtgttggttggattcgtcaaccttctggtaaaggtcttgaatggctttctcatatttggtgggatgatgttaaacgttataatcctgctcttaaatctcgtcttactatttctcgtgatacttcttattctcaagtttttcttcgtattgcttctgttgatactgctgatactgctacttattattgtgctcgtattcttgatggtactggtcctatggattattggggtcaaggtacttctgttactgtttcttctgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 267 - protein, 453 aa, synthetic construct
LLQGSGQVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 268 - DNA, 648 bp, synthetic construct
  source 1..648: mol_type = other DNA
caaattgttcttactcaatctcctgctattatgtctgctagccctggtgaaaaagttactatgacttgttctgcttcttctcgtgttacttatatgcattggtatcaacaacgttctggtacttctcctaaacgttggatttatgatacttctaaacttgcttctggtgttcctgctcgtttttctggttctggttctggtacttcttattctcttactatttcttctatggaagctgaagatgctgctacttattattgtcaacaatggggtaataatcctcaatatacttttggtggtggtactcgtcttgaaattaaacgtcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 269 - protein, 215 aa, synthetic construct
QIVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - DNA, 1344 bp, synthetic construct
  source 1..1344: mol_type = other DNA
caagttactcttaaagaatctggtcctggaattcttcaaccttctcaaactctttctcttacttgttctttttctggtttttctctttctacttctggtactggtgttggttggattcgtcaaccttctggtaaaggtcttgaatggctttctcatatttggtgggatgatgttaaacgttataatcctgctcttaaatctcgtcttactatttctcgtgatacttcttattctcaagtttttcttcgtattgcttctgttgatactgctgatactgctacttattattgtgctcgtattcttgatggtactggtcctatggattattggggtcaaggtacttctgttactgtttcttctgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 271 - protein, 447 aa, synthetic construct
QVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 272 - DNA, 666 bp, synthetic construct
  source 1..666: mol_type = other DNA
ttacttcagggatctggtcaaattgttcttactcaatctcctgctattatgtctgctagccctggtgaaaaagttactatgacttgttctgcttcttctcgtgttacttatatgcattggtatcaacaacgttctggtacttctcctaaacgttggatttatgatacttctaaacttgcttctggtgttcctgctcgtttttctggttctggttctggtacttcttattctcttactatttcttctatggaagctgaagatgctgctacttattattgtcaacaatggggtaataatcctcaatatacttttggtggtggtactcgtcttgaaattaaacgtcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 273 - protein, 221 aa, synthetic construct
LLQGSGQIVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - DNA, 360 bp, synthetic construct
  source 1..360: mol_type = other DNA
gaggtgcagctggtggagtctgggggaggcttggtccagcctggggggtccctgagactctcctgtgcagcctctggattcaccttctctaggtactggatgacctgggtccgccaggctccagggaaggggctggagtgggtggccaacataaagcaagatggcagtgggaaaaactatgtggactctgtgatgggccgatacaccatctccagagacaacgccaagaactcactgtatctgcaaatgaacagcctgagagccgaggacacggctgtgtattactgtgcgagatggatagcaccagatttccccggtatggacgtctggggccaagggaccacggtcaccgtctcctca

SEQ ID NO: 275 - protein, 120 aa, synthetic construct
EVQLVESGGGLVQPGGSLRLSCAASGFTFSRYWMTWVRQAPGKGLEWVANIKQDGSGKNYVDSVMGRYTISRDNAKNSLYLQMNSLRAEDTAVYYCARWIAPDFPGMDVWGQGTTVTVSS

SEQ ID NO: 276 - DNA, 24 bp, synthetic construct
  source 1..24: mol_type = other DNA
ggattcaccttctctaggtactgg

SEQ ID NO: 277 - protein, 8 aa, synthetic construct
GFTFSRYW

SEQ ID NO: 278 - DNA, 24 bp, synthetic construct
  source 1..24: mol_type = other DNA
ataaagcaagatggcagtgggaaa

SEQ ID NO: 279 - protein, 8 aa, synthetic construct
IKQDGSGK

SEQ ID NO: 280 - DNA, 39 bp, synthetic construct
  source 1..39: mol_type = other DNA
gcgagatggatagcaccagatttccccggtatggacgtc

SEQ ID NO: 281 - protein, 13 aa, synthetic construct
ARWIAPDFPGMDV

SEQ ID NO: 282 - DNA, 321 bp, synthetic construct
  source 1..321: mol_type = other DNA
gacatccagttgacccagtctccatccttcctgtctgcatctgtaggagacagagtcaccatcacttgctgggccagtcagggcattagcagttatttagcctggtatcagcaaaaaccaggaaaagcccctaagctcctgatctatgctgcatccactttgcaaagtggggtcccatcaaggttcagcggcagtggatctgggacagaattcactctcacaatcagcagcctgcagcctgcagattttgcaacttattactgtcaacagcttaatagttacccgctcactttcggcggagggaccaaggtggagatcaaa

SEQ ID NO: 283 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPADFATYYCQQLNSYPLTFGGGTKVEIK

SEQ ID NO: 284 - DNA, 18 bp, synthetic construct
  source 1..18: mol_type = other DNA
cagggcattagcagttat

SEQ ID NO: 285 - protein, 6 aa, synthetic construct
QGISSY

SEQ ID NO: 286-287 - skipped (2 entries)

SEQ ID NO: 288 - DNA, 27 bp, synthetic construct
  source 1..27: mol_type = other DNA
caacagcttaatagttacccgctcact

SEQ ID NO: 289 - protein, 9 aa, synthetic construct
QQLNSYPLT

SEQ ID NO: 290 - DNA, 1344 bp, synthetic construct
  source 1..1344: mol_type = other DNA
gaggtgcagctggtggagtctgggggaggcttggtccagcctggggggtccctgagactctcctgtgcagcctctggattcaccttctctaggtactggatgacctgggtccgccaggctccagggaaggggctggagtgggtggccaacataaagcaagatggcagtgggaaaaactatgtggactctgtgatgggccgatacaccatctccagagacaacgccaagaactcactgtatctgcaaatgaacagcctgagagccgaggacacggctgtgtattactgtgcgagatggatagcaccagatttccccggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 291 - protein, 447 aa, synthetic construct
EVQLVESGGGLVQPGGSLRLSCAASGFTFSRYWMTWVRQAPGKGLEWVANIKQDGSGKNYVDSVMGRYTISRDNAKNSLYLQMNSLRAEDTAVYYCARWIAPDFPGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 292 - DNA, 663 bp, synthetic construct
  source 1..663: mol_type = other DNA
ctgctgcaaggctctggcgacatccagttgacccagtctccatccttcctgtctgcatctgtaggagacagagtcaccatcacttgctgggccagtcagggcattagcagttatttagcctggtatcagcaaaaaccaggaaaagcccctaagctcctgatctatgctgcatccactttgcaaagtggggtcccatcaaggttcagcggcagtggatctgggacagaattcactctcacaatcagcagcctgcagcctgcagattttgcaacttattactgtcaacagcttaatagttacccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 293 - protein, 220 aa, synthetic construct
LLQGSGDIQLTQSPSFLSASVGDRVTITCWASQGISSYLAWYQQKPGKAPKLLIYAASTLQSGVPSRFSGSGSGTEFTLTISSLQPADFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - DNA, 366 bp, synthetic construct; identical to SEQ ID NO: 165
  source 1..366: mol_type = other DNA

SEQ ID NO: 295 - protein, 122 aa, synthetic construct; identical to SEQ ID NO: 166

SEQ ID NO: 296 - DNA, 24 bp, synthetic construct
  source 1..24: mol_type = other DNA
gggttcaccttcagtggctctgct

SEQ ID NO: 297 - protein, 8 aa, synthetic construct
GFTFSGSA

SEQ ID NO: 298 - DNA, 30 bp, synthetic construct; identical to SEQ ID NO: 169
  source 1..30: mol_type = other DNA

SEQ ID NO: 299 - protein, 10 aa, synthetic construct
ITSKANSYAT

SEQ ID NO: 300 - DNA, 39 bp, synthetic construct; identical to SEQ ID NO: 171
  source 1..39: mol_type = other DNA

SEQ ID NO: 301 - protein, 13 aa, synthetic construct
TRQRFLEFLFLDY

SEQ ID NO: 302 - DNA, 324 bp, synthetic construct; identical to SEQ ID NO: 173
  source 1..324: mol_type = other DNA

SEQ ID NO: 303 - protein, 108 aa, synthetic construct; identical to SEQ ID NO: 174

SEQ ID NO: 304 - DNA, 18 bp, synthetic construct
  source 1..18: mol_type = other DNA
cagagcattagcagctat

SEQ ID NO: 305 - protein, 6 aa, synthetic construct
QSISSY

SEQ ID NO: 306-307 - skipped (2 entries)

SEQ ID NO: 308 - DNA, 30 bp, synthetic construct; identical to SEQ ID NO: 179
  source 1..30: mol_type = other DNA

SEQ ID NO: 309 - protein, 10 aa, synthetic construct
QQSYSTPPIT

SEQ ID NO: 310 - DNA, 1350 bp, synthetic construct; identical to SEQ ID NO: 181
  source 1..1350: mol_type = other DNA

SEQ ID NO: 311 - protein, 449 aa, synthetic construct; identical to SEQ ID NO: 182

SEQ ID NO: 312 - DNA, 666 bp, synthetic construct; identical to SEQ ID NO: 183
  source 1..666: mol_type = other DNA

SEQ ID NO: 313 - protein, 221 aa, synthetic construct; identical to SEQ ID NO: 184

SEQ ID NO: 314 - DNA, 375 bp, synthetic construct; identical to SEQ ID NO: 145
  source 1..375: mol_type = other DNA

SEQ ID NO: 315 - protein, 125 aa, synthetic construct; identical to SEQ ID NO: 146

SEQ ID NO: 316 - DNA, 23 bp, synthetic construct
  source 1..23: mol_type = other DNA
gattcaccttcagtggctatggc

SEQ ID NO: 317 - protein, 8 aa, synthetic construct
GFTFSGYG

SEQ ID NO: 318 - DNA, 24 bp, synthetic construct
  source 1..24: mol_type = other DNA
atatggtatgatggaagttttaaa

SEQ ID NO: 319 - protein, 8 aa, synthetic construct
IWYDGSFK

SEQ ID NO: 320 - DNA, 54 bp, synthetic construct; identical to SEQ ID NO: 151
  source 1..54: mol_type = other DNA

SEQ ID NO: 321 - protein, 18 aa, synthetic construct
ARGDSSSSGRYYYYGMDV

SEQ ID NO: 322 - DNA, 321 bp, synthetic construct; identical to SEQ ID NO: 153
  source 1..321: mol_type = other DNA

SEQ ID NO: 323 - protein, 107 aa, synthetic construct; identical to SEQ ID NO: 154

SEQ ID NO: 324 - DNA, 18 bp, synthetic construct
  source 1..18: mol_type = other DNA
cagggcattagaaatgat

SEQ ID NO: 325 - protein, 6 aa, synthetic construct
QGIRND

SEQ ID NO: 326-327 - skipped (2 entries)

SEQ ID NO: 328 - DNA, 27 bp, synthetic construct
  source 1..27: mol_type = other DNA
ctacagcataataattaccctcccact

SEQ ID NO: 329 - protein, 9 aa, synthetic construct
LQHNNYPPT

SEQ ID NO: 330 - DNA, 1377 bp, synthetic construct
  source 1..1377: mol_type = other DNA
ctgctgcaaggctctggccaggtgcagctggtggagtctgggggaggcgtggtccagcctgggaggtccctgagactctcctgtgcagcgtctggattcaccttcagtggctatggcatacactgggtccgccaggctccaggcaagggactggtgtgggtggcagttatatggtatgatggaagttttaaatactatgcagactccgtgaagggccgattcaccatctccagagacaattccaagaacacgctgtatctgcagatgaacagcctgagagccgaggacacggctgtgtattactgtgcgagaggtgatagcagctcgtccggacggtactactactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 331 - protein, 458 aa, synthetic construct
LLQGSGQVQLVESGGGVVQPGRSLRLSCAASGFTFSGYGIHWVRQAPGKGLVWVAVIWYDGSFKYYADSVKGRFTISRDNSKNTLYLQMNSLRAEDTAVYYCARGDSSSSGRYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 332 - DNA, 645 bp, synthetic construct
  source 1..645: mol_type = other DNA
gacatccagatgacccagtctccatcctccctgtctgcatctgtaggagacagagtcaccatcacttgccgggcaagtcagggcattagaaatgatttaggctggtatcagcagaaaccagggacagcccctaagcgcctgatctttgctgcatccagtttgcaaagtggggtcccatcaaggttcagcggcagtggatctgggacagaattcactctcacaatcagcagcctgcagcctgaagattttgcgacttattactgtctacagcataataattaccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 333 - protein, 214 aa, synthetic construct
DIQMTQSPSSLSASVGDRVTITCRASQGIRNDLGWYQQKPGTAPKRLIFAASSLQSGVPSRFSGSGSGTEFTLTISSLQPEDFATYYCLQHNNYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - DNA, 372 bp, synthetic construct
  source 1..372: mol_type = other DNA
caggtgcagctacagcagtggggcgcaggactgttgaagccttcggagaccctgtccctcacctgcgctgtctatggtgggtccttcagtggttactactggagctggatccgccagcccccaggaaaggggctggagtggattggggaaatcaatcatgctggaagcaccaactacaacccgtccctcaagagtcgaatcaccatatcagtggacacgtccaagaaccagttctccctgaagctgagttctgtgaccgccgcggacacggctgtgtattactgtgcgagaggatggtactatggttcggggagttatcaccgaaactggttcgacccctggggccagggaaccctggtcaccgtctcctca

SEQ ID NO: 335 - protein, 124 aa, synthetic construct
QVQLQQWGAGLLKPSETLSLTCAVYGGSFSGYYWSWIRQPPGKGLEWIGEINHAGSTNYNPSLKSRITISVDTSKNQFSLKLSSVTAADTAVYYCARGWYYGSGSYHRNWFDPWGQGTLVTVSS

SEQ ID NO: 336 - DNA, 24 bp, synthetic construct
  source 1..24: mol_type = other DNA
ggtgggtccttcagtggttactac

SEQ ID NO: 337 - protein, 8 aa, synthetic construct
GGSFSGYY

SEQ ID NO: 338 - DNA, 21 bp, synthetic construct
  source 1..21: mol_type = other DNA
atcaatcatgctggaagcacc

SEQ ID NO: 339 - protein, 7 aa, synthetic construct
INHAGST

SEQ ID NO: 340 - DNA, 54 bp, synthetic construct
  source 1..54: mol_type = other DNA
gcgagaggatggtactatggttcggggagttatcaccgaaactggttcgacccc

SEQ ID NO: 341 - protein, 18 aa, synthetic construct
ARGWYYGSGSYHRNWFDP

SEQ ID NO: 342 - DNA, 324 bp, synthetic construct
  source 1..324: mol_type = other DNA
gaaattgtattgacgcagtctccaggcaccctgtctttgtctccaggggaaagagccaccctctcctgcagggccagtcagagtgtttattacagctacttagcctggtatcagcagaaacctggccaggctcccaggctcctcatctatggtgcatccaacagggccactggcatcccagacaggttcagtggcagtgggtctgggacagacttcactctcaccatcagcagactggagcctgaagattttgcagtgtattactgtcagcagtatggtaactcaccttggacgttcggccaagggaccaaggtggaaatcaaa

SEQ ID NO: 343 - protein, 108 aa, synthetic construct
EIVLTQSPGTLSLSPGERATLSCRASQSVYYSYLAWYQQKPGQAPRLLIYGASNRATGIPDRFSGSGSGTDFTLTISRLEPEDFAVYYCQQYGNSPWTFGQGTKVEIK

SEQ ID NO: 344 - DNA, 21 bp, synthetic construct
  source 1..21: mol_type = other DNA
cagagtgtttattacagctac

SEQ ID NO: 345 - protein, 7 aa, synthetic construct
QSVYYSY

SEQ ID NO: 346-347 - skipped (2 entries)

SEQ ID NO: 348 - DNA, 27 bp, synthetic construct
  source 1..27: mol_type = other DNA
cagcagtatggtaactcaccttggacg

SEQ ID NO: 349 - protein, 9 aa, synthetic construct
QQYGNSPWT

SEQ ID NO: 350 - DNA, 1374 bp, synthetic construct
  source 1..1374: mol_type = other DNA
ttacttcagggatctggtcaggtgcagctacagcagtggggcgcaggactgttgaagccttcggagaccctgtccctcacctgcgctgtctatggtgggtccttcagtggttactactggagctggatccgccagcccccaggaaaggggctggagtggattggggaaatcaatcatgctggaagcaccaactacaacccgtccctcaagagtcgaatcaccatatcagtggacacgtccaagaaccagttctccctgaagctgagttctgtgaccgccgcggacacggctgtgtattactgtgcgagaggatggtactatggttcggggagttatcaccgaaactggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 351 - protein, 457 aa, synthetic construct
LLQGSGQVQLQQWGAGLLKPSETLSLTCAVYGGSFSGYYWSWIRQPPGKGLEWIGEINHAGSTNYNPSLKSRITISVDTSKNQFSLKLSSVTAADTAVYYCARGWYYGSGSYHRNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 352 - DNA, 648 bp, synthetic construct
  source 1..648: mol_type = other DNA
gaaattgtattgacgcagtctccaggcaccctgtctttgtctccaggggaaagagccaccctctcctgcagggccagtcagagtgtttattacagctacttagcctggtatcagcagaaacctggccaggctcccaggctcctcatctatggtgcatccaacagggccactggcatcccagacaggttcagtggcagtgggtctgggacagacttcactctcaccatcagcagactggagcctgaagattttgcagtgtattactgtcagcagtatggtaactcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 353 - protein, 215 aa, synthetic construct
EIVLTQSPGTLSLSPGERATLSCRASQSVYYSYLAWYQQKPGQAPRLLIYGASNRATGIPDRFSGSGSGTDFTLTISRLEPEDFAVYYCQQYGNSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 354 - DNA, 372 bp, synthetic construct; identical to SEQ ID NO: 334
  source 1..372: mol_type = other DNA

SEQ ID NO: 355 - protein, 124 aa, synthetic construct; identical to SEQ ID NO: 335

SEQ ID NO: 356 - DNA, 24 bp, synthetic construct
  source 1..24: mol_type = other DNA
ggtgggtccttcagtggttactac

SEQ ID NO: 357 - protein, 8 aa, synthetic construct
GGSFSGYY

SEQ ID NO: 358 - DNA, 21 bp, synthetic construct
  source 1..21: mol_type = other DNA
atcaatcatgctggaagcacc

SEQ ID NO: 359 - protein, 7 aa, synthetic construct
INHAGST

SEQ ID NO: 360 - DNA, 54 bp, synthetic construct; identical to SEQ ID NO: 340
  source 1..54: mol_type = other DNA

SEQ ID NO: 361 - protein, 18 aa, synthetic construct
ARGWYYGSGSYHRNWFDP

SEQ ID NO: 362 - DNA, 324 bp, synthetic construct; identical to SEQ ID NO: 342
  source 1..324: mol_type = other DNA

SEQ ID NO: 363 - protein, 108 aa, synthetic construct; identical to SEQ ID NO: 343

SEQ ID NO: 364 - DNA, 21 bp, synthetic construct
  source 1..21: mol_type = other DNA
cagagtgtttattacagctac

SEQ ID NO: 365 - protein, 7 aa, synthetic construct
QSVYYSY

SEQ ID NO: 366-367 - skipped (2 entries)

SEQ ID NO: 368 - DNA, 27 bp, synthetic construct
  source 1..27: mol_type = other DNA
cagcagtatggtaactcaccttggacg

SEQ ID NO: 369 - protein, 9 aa, synthetic construct
QQYGNSPWT

SEQ ID NO: 370 - DNA, 1356 bp, synthetic construct
  source 1..1356: mol_type = other DNA
caggtgcagctacagcagtggggcgcaggactgttgaagccttcggagaccctgtccctcacctgcgctgtctatggtgggtccttcagtggttactactggagctggatccgccagcccccaggaaaggggctggagtggattggggaaatcaatcatgctggaagcaccaactacaacccgtccctcaagagtcgaatcaccatatcagtggacacgtccaagaaccagttctccctgaagctgagttctgtgaccgccgcggacacggctgtgtattactgtgcgagaggatggtactatggttcggggagttatcaccgaaactggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 371 - protein, 451 aa, synthetic construct
QVQLQQWGAGLLKPSETLSLTCAVYGGSFSGYYWSWIRQPPGKGLEWIGEINHAGSTNYNPSLKSRITISVDTSKNQFSLKLSSVTAADTAVYYCARGWYYGSGSYHRNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 372 - DNA, 666 bp, synthetic construct
  source 1..666: mol_type = other DNA
ttacttcagggatctggtgaaattgtattgacgcagtctccaggcaccctgtctttgtctccaggggaaagagccaccctctcctgcagggccagtcagagtgtttattacagctacttagcctggtatcagcagaaacctggccaggctcccaggctcctcatctatggtgcatccaacagggccactggcatcccagacaggttcagtggcagtgggtctgggacagacttcactctcaccatcagcagactggagcctgaagattttgcagtgtattactgtcagcagtatggtaactcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 373 - protein, 221 aa, synthetic construct
LLQGSGEIVLTQSPGTLSLSPGERATLSCRASQSVYYSYLAWYQQKPGQAPRLLIYGASNRATGIPDRFSGSGSGTDFTLTISRLEPEDFAVYYCQQYGNSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 374 - DNA, 378 bp, synthetic construct
  source 1..378: mol_type = other DNA
gaagtgcaggtggtggagtctgggggaggcttggtacagcctggcaggtccctgagactctcctgtacagcctctggattcacctttgatgattatgccatgttttgggtccggcaaggtccagggaagggcctggagtgggtctcaggtattagttggaatagtggtagcataggctatgcggactctgtaaagggccgcttcaccacctccagagacaacgccaagaactccctatatttacaaatgaacagtctgagaactgaagacacggccttgtattactgtgcaaaagattatcgaccccgtagtgggaaccactataacaactacggtatggacgtctggggcccagggaccacggtcaccgtctcctca

SEQ ID NO: 375 - protein, 126 aa, synthetic construct
EVQVVESGGGLVQPGRSLRLSCTASGFTFDDYAMFWVRQGPGKGLEWVSGISWNSGSIGYADSVKGRFTTSRDNAKNSLYLQMNSLRTEDTALYYCAKDYRPRSGNHYNNYGMDVWGPGTTVTVSS

SEQ ID NO: 376 - DNA, 24 bp, synthetic construct
  source 1..24: mol_type = other DNA
ggattcacctttgatgattatgcc

SEQ ID NO: 377 - protein, 8 aa, synthetic construct
GFTFDDYA

SEQ ID NO: 378 - DNA, 24 bp, synthetic construct
  source 1..24: mol_type = other DNA
attagttggaatagtggtagcata

SEQ ID NO: 379 - protein, 8 aa, synthetic construct
ISWNSGSI

SEQ ID NO: 380 - DNA, 57 bp, synthetic construct
  source 1..57: mol_type = other DNA
gcaaaagattatcgaccccgtagtgggaaccactataacaactacggtatggacgtc

SEQ ID NO: 381 - protein, 19 aa, synthetic construct
AKDYRPRSGNHYNNYGMDV

SEQ ID NO: 382 - DNA, 324 bp, synthetic construct
  source 1..324: mol_type = other DNA
gagattgtgttgacgcagtctccaggcaccctgtctttgtctccaggggaaagagccaccctctcctgcagggccagtcagagttttcgcggcaactacttagcctggtaccagcagaaacctggccaggctcccagactcctcatctatggtgcatccagcagggccactggcatcccagacaggttcaggggcagtgggtctgggacagacttcacgctcaccatcagcagactggagcctgaggattttgcagtatattactgtcaccagtatggtaggtcaccttggacgttcggccaagggaccaaggtggaaatcaaa

SEQ ID NO: 383 - protein, 108 aa, synthetic construct
EIVLTQSPGTLSLSPGERATLSCRASQSFRGNYLAWYQQKPGQAPRLLIYGASSRATGIPDRFRGSGSGTDFTLTISRLEPEDFAVYYCHQYGRSPWTFGQGTKVEIK

SEQ ID NO: 384 - DNA, 21 bp, synthetic construct
  source 1..21: mol_type = other DNA
cagagttttcgcggcaactac

SEQ ID NO: 385 - protein, 7 aa, synthetic construct
QSFRGNY

SEQ ID NO: 386-387 - skipped (2 entries)

SEQ ID NO: 388 - DNA, 27 bp, synthetic construct
  source 1..27: mol_type = other DNA
caccagtatggtaggtcaccttggacg

SEQ ID NO: 389 - protein, 9 aa, synthetic construct
HQYGRSPWT

SEQ ID NO: 390 - DNA, 1380 bp, synthetic construct
  source 1..1380: mol_type = other DNA
ttacttcagggatctggtgaagtgcaggtggtggagtctgggggaggcttggtacagcctggcaggtccctgagactctcctgtacagcctctggattcacctttgatgattatgccatgttttgggtccggcaaggtccagggaagggcctggagtgggtctcaggtattagttggaatagtggtagcataggctatgcggactctgtaaagggccgcttcaccacctccagagacaacgccaagaactccctatatttacaaatgaacagtctgagaactgaagacacggccttgtattactgtgcaaaagattatcgaccccgtagtgggaaccactataacaactacggtatggacgtctggggcccagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 391 - protein, 459 aa, synthetic construct
LLQGSGEVQVVESGGGLVQPGRSLRLSCTASGFTFDDYAMFWVRQGPGKGLEWVSGISWNSGSIGYADSVKGRFTTSRDNAKNSLYLQMNSLRTEDTALYYCAKDYRPRSGNHYNNYGMDVWGPGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 392 - DNA, 648 bp, synthetic construct
  source 1..648: mol_type = other DNA
gagattgtgttgacgcagtctccaggcaccctgtctttgtctccaggggaaagagccaccctctcctgcagggccagtcagagttttcgcggcaactacttagcctggtaccagcagaaacctggccaggctcccagactcctcatctatggtgcatccagcagggccactggcatcccagacaggttcaggggcagtgggtctgggacagacttcacgctcaccatcagcagactggagcctgaggattttgcagtatattactgtcaccagtatggtaggtcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 393 - protein, 215 aa, synthetic construct
EIVLTQSPGTLSLSPGERATLSCRASQSFRGNYLAWYQQKPGQAPRLLIYGASSRATGIPDRFRGSGSGTDFTLTISRLEPEDFAVYYCHQYGRSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 394 - DNA, 378 bp, synthetic construct; identical to SEQ ID NO: 374
  source 1..378: mol_type = other DNA

SEQ ID NO: 395 - protein, 126 aa, synthetic construct; identical to SEQ ID NO: 375

SEQ ID NO: 396 - DNA, 24 bp, synthetic construct
  source 1..24: mol_type = other DNA
ggattcacctttgatgattatgcc

SEQ ID NO: 397 - protein, 8 aa, synthetic construct
GFTFDDYA

SEQ ID NO: 398 - DNA, 24 bp, synthetic construct
  source 1..24: mol_type = other DNA
attagttggaatagtggtagcata

SEQ ID NO: 399 - protein, 8 aa, synthetic construct
ISWNSGSI

SEQ ID NO: 400 - DNA, 57 bp, synthetic construct; identical to SEQ ID NO: 380
  source 1..57: mol_type = other DNA

SEQ ID NO: 401 - protein, 19 aa, synthetic construct
AKDYRPRSGNHYNNYGMDV

SEQ ID NO: 402 - DNA, 324 bp, synthetic construct; identical to SEQ ID NO: 382
  source 1..324: mol_type = other DNA

SEQ ID NO: 403 - protein, 108 aa, synthetic construct; identical to SEQ ID NO: 383

SEQ ID NO: 404 - DNA, 21 bp, synthetic construct
  source 1..21: mol_type = other DNA
cagagttttcgcggcaactac

SEQ ID NO: 405 - protein, 7 aa, synthetic construct
QSFRGNY

SEQ ID NO: 406-407 - skipped (2 entries)

SEQ ID NO: 408 - DNA, 27 bp, synthetic construct
  source 1..27: mol_type = other DNA
caccagtatggtaggtcaccttggacg

SEQ ID NO: 409 - protein, 9 aa, synthetic construct
HQYGRSPWT

SEQ ID NO: 410 - DNA, 1362 bp, synthetic construct
  source 1..1362: mol_type = other DNA
gaagtgcaggtggtggagtctgggggaggcttggtacagcctggcaggtccctgagactctcctgtacagcctctggattcacctttgatgattatgccatgttttgggtccggcaaggtccagggaagggcctggagtgggtctcaggtattagttggaatagtggtagcataggctatgcggactctgtaaagggccgcttcaccacctccagagacaacgccaagaactccctatatttacaaatgaacagtctgagaactgaagacacggccttgtattactgtgcaaaagattatcgaccccgtagtgggaaccactataacaactacggtatggacgtctggggcccagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 411 - protein, 453 aa, synthetic construct
EVQVVESGGGLVQPGRSLRLSCTASGFTFDDYAMFWVRQGPGKGLEWVSGISWNSGSIGYADSVKGRFTTSRDNAKNSLYLQMNSLRTEDTALYYCAKDYRPRSGNHYNNYGMDVWGPGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 412 - DNA, 666 bp, synthetic construct
  source 1..666: mol_type = other DNA
ttacttcagggatctggtgagattgtgttgacgcagtctccaggcaccctgtctttgtctccaggggaaagagccaccctctcctgcagggccagtcagagttttcgcggcaactacttagcctggtaccagcagaaacctggccaggctcccagactcctcatctatggtgcatccagcagggccactggcatcccagacaggttcaggggcagtgggtctgggacagacttcacgctcaccatcagcagactggagcctgaggattttgcagtatattactgtcaccagtatggtaggtcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 413 - protein, 221 aa, synthetic construct
LLQGSGEIVLTQSPGTLSLSPGERATLSCRASQSFRGNYLAWYQQKPGQAPRLLIYGASSRATGIPDRFRGSGSGTDFTLTISRLEPEDFAVYYCHQYGRSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 414 - protein, 447 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 415 - DNA, 1344 bp, synthetic construct
  source 1..1344: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 416 - protein, 446 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 417 - DNA, 1341 bp, synthetic construct
  source 1..1341: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggt

SEQ ID NO: 418 - protein, 8 aa, synthetic construct
SLSLSLGK

SEQ ID NO: 419 - protein, 9 aa, synthetic construct
GGGGSGGGG

SEQ ID NO: 420 - protein, 113 aa, synthetic construct
LLQGSGDIQMTQSPSSVSASVGDRVTITCRASQGINSWLAWYQQKPGKAPKLLIYAASSLQSGVPSRFSGSGSGTDFTLTISSLQPEDFATYYCHQADSFPYTFGQGTKLEIK

SEQ ID NO: 421 - protein, 113 aa, synthetic construct
LLQGSGDIQMTQSPSSVSASVGDRVTITCRASQGINSWLAWYQQKPGKAPKLLIYAASSLESGVPSRFSGSGSGTDFTLTISSLQPEDFATYYCHQADSFPYTFGQGTKLEIK

SEQ ID NO: 422 - protein, 118 aa, synthetic construct
LLQGSGDIVMTQSPLSSPVTLGQPASISCRSSQSLVHFDGNTYLSWLHQRPGQPPRLLIYKISNRFSGVPDRFSGSGAGTDFTLKISRVEPEDVGVYYCMHATQFPYTFGQGTKLEIK

SEQ ID NO: 423 - protein, 113 aa, synthetic construct
LLQGSGDIQLTQSPSFLSASVGDRVTITCWASQGISSYLAWYQQKPGKAPKLLIYAASTLQSGVPSRFSGSGSGTEFTLTISSLQPEDFALYYCQQLNSYPRTFGQGTKVEIK

SEQ ID NO: 424 - protein, 125 aa, synthetic construct
LLQGSGEVQLVESGGGLVKPGGSLRLSCAASGFIFSRYSMNWVRQAPGKGLEWVSSMSSNSKNTYYADSVKGRFTISRDNAKNSLFLQMNTLRAEDTAVYYCARDGYTLRAFDIWGQGTMVTVSS

SEQ ID NO: 425 - protein, 113 aa, synthetic construct
LLQGSGDIQMTQSPSSLSASVGDRVTITCRASQGIRNDLGWYQQKPGTAPKRLIFAASSLQSGVPSRFSGSGSGTEFTLTISSLQPEDFATYYCLQHNNYPPTFGGGTKVEIK

SEQ ID NO: 426 - protein, 114 aa, synthetic construct
LLQGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 427 - protein, 127 aa, synthetic construct
LLQGSGEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSS

SEQ ID NO: 428 - protein, 122 aa, synthetic construct
LLQGSGQVQLVESGGGVGQPGRSLRLSCAASGFTFSRNAMHWVRQAPGKGLEWVAVISYDGSNKHYADSVKGRFTISRDNSKNTLYLEMNSLRVEDTAVYYCAKGGIPFDYWGQGTLVTVSS

SEQ ID NO: 429 - protein, 125 aa, synthetic construct
LLQGSGQVQLVESGGGVVQPARSLRLSCAASGFAFSRSAMHWVRQAPGKGLEWVAVISYDGSNKYYTDSVKGRFTISRDNSKNTLYLQMNTLRAEDTALYYCAKMYTTMDSFDYWGQGTLVTVSS

SEQ ID NO: 430 - protein, 128 aa, synthetic construct
LLQGSGEVQLVESGGGLVQPGGSLKLSCAASGFTFSGSAMHWVRQASGKGLEWVGRITSKANSYATAYDASVKGRFTISRDDSKNTAYLQMNSLKTEDTAVYYCTRQRFLEFLFLDYWGQGTLVTVSS

SEQ ID NO: 431 - protein, 113 aa, synthetic construct
LLQGSGDIQLTQSPSFLSASVGDRVTITCWASQGISSYLAWYQQKPGKAPKLLIYAASTLQSGVPSRFSGSGSGTEFTLTISSLQPADFATYYCQQLNSYPLTFGGGTKVEIK

SEQ ID NO: 432 - protein, 114 aa, synthetic construct; identical to SEQ ID NO: 426

SEQ ID NO: 433 - protein, 131 aa, synthetic construct
LLQGSGQVQLVESGGGVVQPGRSLRLSCAASGFTFSGYGIHWVRQAPGKGLVWVAVIWYDGSFKYYADSVKGRFTISRDNSKNTLYLQMNSLRAEDTAVYYCARGDSSSSGRYYYYGMDVWGQGTTVTVSS

SEQ ID NO: 434 - protein, 130 aa, synthetic construct
LLQGSGQVQLQQWGAGLLKPSETLSLTCAVYGGSFSGYYWSWIRQPPGKGLEWIGEINHAGSTNYNPSLKSRITISVDTSKNQFSLKLSSVTAADTAVYYCARGWYYGSGSYHRNWFDPWGQGTLVTVSS

SEQ ID NO: 435 - protein, 114 aa, synthetic construct
LLQGSGEIVLTQSPGTLSLSPGERATLSCRASQSVYYSYLAWYQQKPGQAPRLLIYGASNRATGIPDRFSGSGSGTDFTLTISRLEPEDFAVYYCQQYGNSPWTFGQGTKVEIK

SEQ ID NO: 436 - protein, 132 aa, synthetic construct
LLQGSGEVQVVESGGGLVQPGRSLRLSCTASGFTFDDYAMFWVRQGPGKGLEWVSGISWNSGSIGYADSVKGRFTTSRDNAKNSLYLQMNSLRTEDTALYYCAKDYRPRSGNHYNNYGMDVWGPGTTVTVSS

SEQ ID NO: 437 - protein, 114 aa, synthetic construct
LLQGSGEIVLTQSPGTLSLSPGERATLSCRASQSFRGNYLAWYQQKPGQAPRLLIYGASSRATGIPDRFRGSGSGTDFTLTISRLEPEDFAVYYCHQYGRSPWTFGQGTKVEIK

SEQ ID NO: 438 - protein, 8 aa, synthetic construct
GGSGGSGG

SEQ ID NO: 439 - protein, 6 aa, synthetic construct
EEQYNS

SEQ ID NO: 440 - protein, 6 aa, synthetic construct
EEQFNS

SEQ ID NO: 441 - protein, 6 aa, synthetic construct
HHHHHH

SEQ ID NO: 442 - protein, 16 aa, synthetic construct
MKTIIALSYIFCLVFA

SEQ ID NO: 443 - protein, 7 aa, synthetic construct
DYKDDDD

SEQ ID NO: 444 - protein, 8 aa, synthetic construct
LEVLFQGP

SEQ ID NO: 445 - protein, 8 aa, synthetic construct
HHHHHHHH

SEQ ID NO: 446 - protein, 4 aa, synthetic construct
GSSG

SEQ ID NO: 447 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanamide
XEGTFTSDXX

SEQ ID NO: 448 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  VARIANT 8: note = (CH2)2-6-NH-, -(CH2)2-6-Tr-, or -(CH2)2-6-Tr-( CH2)1-6-NH attached to (S)-2-amino-3-(2'-ethyl-4'-methoxy- [1,1'-biphenyl]-4-yl)propanoic acid linker
  VARIANT 9: note = (S)-2-amino-5-(3,5-dimethylphenyl)pentanoic acid or optionally substituted (S)-2-amino-5-phenylpentanoic acid attached to amine, hydroxyl or phenylamine
  VARIANT 9: note = May be absent
XGTXTSDXX

SEQ ID NO: 449 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid
  VARIANT 9: note = (CH2)2-6-NH-, -(CH2)2-6-Tr-, or -(CH2)2-6-Tr-( CH2)1-6-NH attached to (S)-2-amino-5-phenylpentanoic acid linker attached to amine, hydroxyl or phenylamine
XGTXTSDXX

SEQ ID NO: 450 - protein, 24 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 7: note = (S)-2-amino-3-(1H-tetrazol-5-yl)propanoic acid
  SITE 11: note = 2-amino-2-methylpropanoic acid
  SITE 13: note = (S)-2-amino-3-(1H-tetrazol-5-yl)propanoic acid
  VARIANT 18: note = (CH2)2-6-NH-, -(CH2)2-6-Tr-, or -(CH2)2-6-Tr-( CH2)1-6-NH attached to 2-amino-2-lambda-3-ethanoic acid
  SITE 20: note = (S)-2-amino-3-(2-fluorophenyl)-2-methylpropanoic acid
  SITE 24: note = 2-aminoacetamide
XGTXTSXYSIXLXKIAQXAXVQGX

SEQ ID NO: 451 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  VARIANT 8: note = (CH2)2-6-NH- or -(CH2)2-6-Tr- attached to (S)-2-ami no-3-(2'-ethyl-4'-methoxy-[1,1'-biphenyl]-4-yl)propanoic acid linker
  VARIANT 9: note = (S)-2-amino-5-(3,5-dimethylphenyl)pentanoic acid attached to amine or phenylamine
  VARIANT 9: note = May be absent
XGTXTSDXX

SEQ ID NO: 452 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid
  VARIANT 9: note = (CH2)2-6-NH- or -(CH2)2-6-Tr- attached to (S)-2-amino-5-phenylpentanoic acid linker attached to amine or phenylamine
XGTXTSDXX

SEQ ID NO: 453 - protein, 7 aa, synthetic construct
  SITE 1: note = 9H-1-lambda-2,8-lambda-2-dibenzo[b,f][1,2,3]triazol o[4,5-d]azocine
  SITE 2: note = oxopentanoic acid
XXGGGGS

SEQ ID NO: 454 - protein, 7 aa, synthetic construct
  SITE 1: note = 9H-1-lambda-2,8-lambda-2-dibenzo[b,f][1,2,3]triazol o[4,5-d]azocine
  SITE 2: note = oxopentanoic acid
XXSGGGG

SEQ ID NO: 455 - protein, 12 aa, synthetic construct
  SITE 1: note = 9H-1-lambda-2,8-lambda-2-dibenzo[b,f][1,2,3]triazol o[4,5-d]azocine
  SITE 2: note = oxopentanoic acid
XXGGGGSGGGGS

SEQ ID NO: 456 - protein, 7 aa, synthetic construct
  SITE 1: note = (((5aR,6S,6aS)-5,5a,6,6a,7,8-hexahydro-4H-1-lambda- 2-cyclopropa[5,6]cycloocta[1,2-d][1,2,3]triazol-6-yl)metho xy)-lambda-3-methanone
  SITE 3: note = glucosyl serine
XGXGGGG

SEQ ID NO: 457 - protein, 6 aa, synthetic construct
  SITE 1: note = 2-((4,5,6,7,8,9-hexahydro-1-lambda-2-cycloocta[d][1 ,2,3]triazol-4-yl)oxy)-1-lambda-3-ethan-1-one
XGGGGS

SEQ ID NO: 458 - protein, 7 aa, synthetic construct
  SITE 1: note = 9H-1-lambda-2,8-lambda-2-dibenzo[b,f][1,2,3]triazol o[4,5-d]azocine
  SITE 2: note = oxopentanoic acid
  SITE 3: note = glucosyl serine
XXXGGGG

SEQ ID NO: 459 - protein, 6 aa, synthetic construct
GGSEPL

SEQ ID NO: 460 - protein, 6 aa, synthetic construct
  SITE 1: note = benzyl hydrogen carbonate Leucine
LLQGSG

SEQ ID NO: 461 - protein, 8 aa, synthetic construct
  SITE 2: note = 2-(2-(2-aminoethoxy)ethoxy)acetic acid
  SITE 3: note = 2-(2-(2-aminoethoxy)ethoxy)acetic acid
EXXGGGGS

SEQ ID NO: 462 - protein, 12 aa, synthetic construct
  SITE 2: note = 2-(2-(2-aminoethoxy)ethoxy)acetic acid
  SITE 3: note = 2-(2-(2-aminoethoxy)ethoxy)acetic acid
EXXGGGGSGGGG

SEQ ID NO: 463 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid linker
  SITE 9: note = (S)-2-amino-5-(3,5-dimethylphenyl)pentanoic acid
XGTXTSDXX

SEQ ID NO: 464 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid
  SITE 9: note = (S)-2-amino-5-phenylpentanoic acid linker
XGTXTSDXX

SEQ ID NO: 465 - protein, 9 aa, synthetic construct
  SITE 1: note = (S)-2-amino-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-azidobutoxy)-2'-ethyl-[1,1'-bi phenyl]-4-yl)propanoic acid
  SITE 9: note = (S)-2-amino-5-(3,5-dimethylphenyl)pentanamide
XGTXTSDXX

SEQ ID NO: 466 - protein, 10 aa, synthetic construct
  SITE 1: note = 2,2-dimethylmalonic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 467 - protein, 10 aa, synthetic construct
  SITE 1: note = 3-amino-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 468 - protein, 10 aa, synthetic construct
  SITE 1: note = 3-(hydroxyamino)-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 469 - protein, 10 aa, synthetic construct
  SITE 1: note = 2-methyl-2-(1H-pyrazol-5-yl)propanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 470 - protein, 10 aa, synthetic construct
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
SXGTXTSDXX

SEQ ID NO: 471 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 472 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 473 - protein, 11 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
HAXGTXTSDXX

SEQ ID NO: 474 - protein, 10 aa, synthetic construct
  SITE 1: note = 6-(1H-imidazol-5-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 475 - protein, 11 aa, synthetic construct
  SITE 1: note = 3-(1H-imidazol-4-yl)propanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
XGXGTXTSDXX

SEQ ID NO: 476 - protein, 12 aa, synthetic construct
  SITE 4: note = (S)-2-amino-3-(2H-tetrazol-5-yl)propanoic acid
  SITE 7: note = (S)-2-amino-3-(2-fluorophenyl)-2-methylpropanoic acid
  SITE 11: note = (S)-2-amino-3-(4'-(4-aminobutoxy)-2'-ethyl-[1,1'-bi phenyl]-4-yl)propanoic acid
  SITE 12: note = (S)-2-amino-5-(3,5-dimethylphenyl)pentanamide
QHAXGTXTSDXX

SEQ ID NO: 477 - protein, 10 aa, synthetic construct
  SITE 1: note = (R)-1-(2-(1H-imidazol-5-yl)ethyl)-3-methyl-2-oxopyr rolidine-3-carboxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 478 - protein, 10 aa, synthetic construct
  SITE 1: note = (S)-1-(2-(1H-imidazol-5-yl)ethyl)-3-methyl-2-oxopyr rolidine-3-carboxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 479 - protein, 10 aa, synthetic construct
  SITE 1: note = (S)-1-(2-(1H-imidazol-5-yl)ethyl)pyrrolidine-3-carb oxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 480 - protein, 10 aa, synthetic construct
  SITE 1: note = (R)-1-(2-(1H-imidazol-5-yl)ethyl)pyrrolidine-3-carb oxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 481 - protein, 11 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
YAXGTXTSDXX

SEQ ID NO: 482 - protein, 11 aa, synthetic construct
  SITE 2: note = 3-amino-2,2-dimethyl-3-oxopropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
YXXGTXTSDXX

SEQ ID NO: 483 - protein, 9 aa, synthetic construct
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanamide
QGTXTSDXX

SEQ ID NO: 484 - protein, 10 aa, synthetic construct
  SITE 1: note = 3-((2-(1H-imidazol-5-yl)ethyl)amino)-2,2-dimethyl-3 -oxopropanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XQGTXTSDXX

SEQ ID NO: 485 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid aniline
XXGTXTSDX

SEQ ID NO: 486 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid aniline
XXGTXTSDXX

SEQ ID NO: 487 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid
  SITE 10: note = (S)-2-amino-5-(4-(4-aminobutoxy)phenyl)pentanamide
XXGTXTSDXX

SEQ ID NO: 488 - protein, 11 aa, synthetic construct
  SITE 1: note = 3-(1H-imidazol-5-yl)propanoic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 489 - protein, 11 aa, synthetic construct
  SITE 1: note = 4-(1H-imidazol-4-yl)butanoic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 490 - protein, 11 aa, synthetic construct
  SITE 1: note = 5-(1H-imidazol-4-yl)pentanoic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 491 - protein, 11 aa, synthetic construct
  SITE 1: note = 6-(1H-imidazol-4-yl)hexanoic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 492 - protein, 11 aa, synthetic construct
  SITE 1: note = 7-(1H-imidazol-4-yl)heptonic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 493 - protein, 11 aa, synthetic construct
  SITE 1: note = 9-(1H-imidazol-5-yl)nonanoic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 494 - protein, 11 aa, synthetic construct
  SITE 1: note = 8-(1H-imidazol-4-yl)octanoic acid
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 495 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(aminomethyl)phenyl)pentanamide linker attached to another sequence
XXGTXTSDXX

SEQ ID NO: 496 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-N-(4-(4,5-diamino-5-oxopentyl)benzyl)-1-hydroxy -3,6,9,12-tetraoxapentadecan-15-amide
XXGTXTSDXX

SEQ ID NO: 497 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(aminomethyl)phenyl)pentanamide linker attached to another sequence
XXGTXTSDXX

SEQ ID NO: 498 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(29-hydroxy-6,9,12,15,18,21,24,27- octaoxa-2-azanonacosyl)phenyl)pentanamide
XXGTXTSDXX

SEQ ID NO: 499 - protein, 10 aa, synthetic construct
  SITE 1: note = 2,2-dimethyl-4-oxo-4-((2-(2-oxopiperidin-1-yl)ethyl )amino)butanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 500 - protein, 10 aa, synthetic construct
  SITE 1: note = 2-((2-((2-(5-methyl-1,3-dioxoisoindolin-2-yl)ethyl) amino)-2-oxoethyl)thio)acetic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 501 - protein, 11 aa, synthetic construct
  SITE 1: note = 3-(2-oxopyrrolidin-1-yl)propanoic acid
  SITE 2: note = 3-aminopropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-azidobutoxy)-2'-ethyl-[1,1'-bi phenyl]-4-yl)propanoic acid
  SITE 11: note = (S)-2-amino-5-(3,5-dimethylphenyl)pentanamide
XXXGTXTSDXX

SEQ ID NO: 502 - protein, 11 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 11: note = (S)-2-amino-5-(3,5-dimethylphenyl)pentanamide
YXXGTXTSDYX

SEQ ID NO: 503 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(1H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid
  SITE 10: note = (S)-2-amino-5-(3,5-dimethylphenyl)pentanamide
XXGTXTSDXX

SEQ ID NO: 504 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 505 - protein, 25 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 8: note = (S)-2-amino-3-(1H-tetrazol-5-yl)propanoic acid
  SITE 12: note = 2-amino-2-methylpropanoic acid
  SITE 14: note = (S)-2-amino-3-(1H-tetrazol-5-yl)propanoic acid
  SITE 19: note = 2-amino-2-lambda-3-ethanoic acid
  SITE 21: note = (S)-2-amino-3-(2-fluorophenyl)-2-methylpropanoic acid
  SITE 25: note = 2-aminoacetamide
XXGTXTSXYSIXLXKIAQXAXVQGX

SEQ ID NO: 506 - protein, 5 aa, synthetic construct
GSGLL

SEQ ID NO: 507 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid linker
  SITE 10: note = (S)-2-amino-5-(3,5-dimethylphenyl)pentanamide
XXGTXTSDXX

SEQ ID NO: 508 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  VARIANT 8: note = CH3, -(CH2)2-6-NH2, -(CH2)2-6-N3, or -(CH2)2-6-Tr-( CH2)1-6-NH2 attached to substituted (S)-2-amino-3-(2'-ethy l-4'-methoxy-[1,1'-biphenyl]-4-yl)propanoic acid
  VARIANT 9: note = (S)-2-amino-5-(3,5-dimethylphenyl)pentanoic acid or optionally substituted (S)-2-amino-5-phenylpentanoic acid attached to amine, hydroxyl or phenylamine
  VARIANT 9: note = May be absent
XGTXTSDXX

SEQ ID NO: 509 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid
  VARIANT 9: note = CH3, -(CH2)2-6-NH2, -(CH2)2-6-N3, or -(CH2)2-6-Tr-( CH2)1-6-NH2 attached to substituted (S)-2-amino-5-phenylpentanoic acid attached to amine, hydroxyl or phenylamine
XGTXTSDXX

SEQ ID NO: 510 - protein, 24 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 7: note = (S)-2-amino-3-(1H-tetrazol-5-yl)propanoic acid
  SITE 11: note = 2-amino-2-methylpropanoic acid
  SITE 13: note = (S)-2-amino-3-(1H-tetrazol-5-yl)propanoic acid
  VARIANT 18: note = CH3, -(CH2)2-6-NH2, -(CH2)2-6-N3, or -(CH2)2-6-Tr-( CH2)1-6-NH2 attached to 2-amino-2-lambda-3-ethanoic acid
  SITE 20: note = (S)-2-amino-3-(2-fluorophenyl)-2-methylpropanoic acid
  SITE 24: note = 2-aminoacetamide
XGTXTSXYSIXLXKIAQXAXVQGX

SEQ ID NO: 511 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  VARIANT 8: note = (CH2)2-6-NH2, -(CH2)2-6-N3, or -CH3 attached to Substituted (S)-2-amino-3-(2'-ethyl-4'-methoxy-[1,1'-biphe nyl]-4-yl)propanoic acid
  VARIANT 9: note = substituted (S)-2-amino-5-phenylpentanoic acid attached to amine or phenylamine
  VARIANT 9: note = May be absent
XGTXTSDXX

SEQ ID NO: 512 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid linker
  VARIANT 9: note = (S)-2-amino-5-(3,5-dimethylphenyl)pentanoic acid or optionally substituted (S)-2-amino-5-phenylpentanoic acid attached to amine, hydroxyl or phenylamine
  VARIANT 9: note = May be absent
XGTXTSDXX

SEQ ID NO: 513 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid linker
  VARIANT 9: note = substituted (S)-2-amino-5-phenylpentanoic acid attached to amine or phenylamine
  VARIANT 9: note = May be absent
XGTXTSDXX

SEQ ID NO: 514 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = pegylated (S)-2-amino-3-(2'-ethyl-4'-methoxy-[1,1'- biphenyl]-4-yl)propanoic acid linker
  SITE 10: note = (S)-2-amino-5-(3,5-dimethylphenyl)pentanamide
XXGTXTSDXX

SEQ ID NO: 515 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 516 - protein, 5 aa, synthetic construct
GGGGS

SEQ ID NO: 517 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 518 - protein, 5 aa, synthetic construct
SGGGG

SEQ ID NO: 519 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker
  SITE 10: note = (S)-2-amino-5-(3,5-dimethylphenyl)pentanamide
XXGTXTSDXX

SEQ ID NO: 520 - protein, 10 aa, synthetic construct
  SITE 1: note = 6-(1H-imidazol-4-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanamide
XXGTXTSDXX

SEQ ID NO: 521 - protein, 10 aa, synthetic construct
  SITE 1: note = 6-(1H-imidazol-4-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker
  SITE 10: note = (S)-2-amino-5-(3,5-dimethylphenyl)pentanamide
XXGTXTSDXX

SEQ ID NO: 522 - protein, 10 aa, synthetic construct
  SITE 1: note = 3-amino-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker
  SITE 10: note = (S)-2-amino-5-(3,5-dimethylphenyl)pentanamide
XXGTXTSDXX

SEQ ID NO: 523 - protein, 10 aa, synthetic construct
  SITE 1: note = 3-amino-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 524 - protein, 5 aa, synthetic construct
GGGGS

SEQ ID NO: 525 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide aniline
XXGTXTSDXX

SEQ ID NO: 526 - protein, 5 aa, synthetic construct
GGGGS

SEQ ID NO: 527 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide aniline
XXGTXTSDXX

SEQ ID NO: 528 - protein, 5 aa, synthetic construct
SGGGG

SEQ ID NO: 529 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 530 - protein, 10 aa, synthetic construct
GGGGSGGGGS

SEQ ID NO: 531 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 532 - protein, 4 aa, synthetic construct
GGGG

SEQ ID NO: 533 - protein, 9 aa, synthetic construct
  SITE 1: note = pegylated Glycine
GGGGSGGGG

SEQ ID NO: 534 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 535 - protein, 5 aa, synthetic construct
  SITE 1: note = Glucosyl Serine
SGGGG

SEQ ID NO: 536 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid
  SITE 10: note = (S)-2-amino-5-phenylpentanamide linker
XXGTXTSDXX

SEQ ID NO: 537 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker
  SITE 10: note = (S)-2-amino-5-phenylpentanamide linker
XXGTXTSDXX

SEQ ID NO: 538 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker
  SITE 10: note = (S)-2-amino-5-(3,5-dimethylphenyl)pentanamide
XXGTXTSDXX

SEQ ID NO: 539 - protein, 10 aa, synthetic construct
  SITE 1: note = 6-(1H-imidazol-4-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker attached to another sequence
  SITE 10: note = (S)-2-amino-5-(3,5-dimethylphenyl)pentanamide
XXGTXTSDXX

SEQ ID NO: 540 - protein, 5 aa, synthetic construct
GGGGS

SEQ ID NO: 541 - protein, 10 aa, synthetic construct
  SITE 1: note = 6-(1H-imidazol-4-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid linker
  SITE 10: note = (S)-2-amino-5-(3,5-dimethylphenyl)pentanamide
XXGTXTSDXX

SEQ ID NO: 542 - protein, 11 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 11: note = (S)-2-amino-5-(3,5-dimethylphenyl)pentanamide
YXXGTXTSDXX

SEQ ID NO: 543 - protein, 10 aa, synthetic construct
  SITE 1: note = 6-(1H-imidazol-4-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-N-(4-(4,5-diamino-5-oxopentyl)benzyl)-1-hydroxy -3,6,9,12,15,18,21,24-octaoxaheptacosan-27-amide
XXGTXTSDXX

SEQ ID NO: 544 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 545 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid linker attached to another sequence
  SITE 10: note = (S)-2-amino-5-(3,5-dimethylphenyl)pentanamide
XXGTXTSDXX

SEQ ID NO: 546 - protein, 6 aa, synthetic construct
GGTEPL

SEQ ID NO: 547 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid linker attached to another sequence
  SITE 10: note = (S)-2-amino-5-(3,5-dimethylphenyl)pentanamide
XXGTXTSDXX

SEQ ID NO: 548 - protein, 6 aa, synthetic construct
  SITE 1: note = benzyl hydrogen carbonate Leucine
LLQGSG

SEQ ID NO: 549 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 550 - protein, 5 aa, synthetic construct
GGGGS

SEQ ID NO: 551 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 552 - protein, 5 aa, synthetic construct
SGGGG

SEQ ID NO: 553 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 554 - protein, 8 aa, synthetic construct
GGSGGSGG

SEQ ID NO: 555 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 556 - protein, 9 aa, synthetic construct
GGGGSGGGG

SEQ ID NO: 557 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 558 - protein, 14 aa, synthetic construct
GGGGSGGGGSGGGG

SEQ ID NO: 559 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 560 - protein, 11 aa, synthetic construct
GGSGGSGGSGG

SEQ ID NO: 561 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 562 - protein, 5 aa, synthetic construct
  SITE 1: note = pegylated Glycine
GGGGS

SEQ ID NO: 563 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-3-(4'-(4-(1H-1,2,3-triazol-1-yl)butoxy)-2'-ethy l-[1,1'-biphenyl]-4-yl)-2-aminopropanoic acid linker attached to another sequence
  SITE 10: note = (S)-2-amino-5-(3,5-dimethylphenyl)pentanamide
XXGTXTSDXX

SEQ ID NO: 564 - protein, 9 aa, synthetic construct
  SITE 1: note = pegylated Glycine
GGGGSGGGS

SEQ ID NO: 565 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = pegylated (S)-3-(4'-(4-(1H-1,2,3-triazol-1-yl)butox y)-2'-ethyl-[1,1'-biphenyl]-4-yl)-2-aminopropanoic acid linker
  SITE 10: note = (S)-2-amino-5-(3,5-dimethylphenyl)pentanamide
XXGTXTSDXX

SEQ ID NO: 566 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = pegylated (S)-3-(4'-(4-(1H-1,2,3-triazol-1-yl)butox y)-2'-ethyl-[1,1'-biphenyl]-4-yl)-2-aminopropanoic acid linker
  SITE 10: note = (S)-N-(4-(4,5-diamino-5-oxopentyl)benzyl)-1-hydroxy -3,6,9,12,15,18,21,24-octaoxaheptacosan-27-amide
XXGTXTSDXX

SEQ ID NO: 567 - protein, 15 aa, synthetic construct
  SITE 1: note = oxopentanoic acid
  VARIANT 2..11: note = This region may encompass 1-10 residues
  VARIANT 12: note = Ser or glucosyl serine
  VARIANT 13: note = Ser or glucosyl serine
  VARIANT 14: note = Ser or glucosyl serine
  VARIANT 15: note = Ser or glucosyl serine
  VARIANT 12..15: note = This region may encompass 0-4 residues
XGGGGGGGGGGXXXX

SEQ ID NO: 568 - protein, 15 aa, synthetic construct
  SITE 1: note = oxopentanoic acid
  VARIANT 2: note = Ser or glucosyl serine
  VARIANT 2..5: note = This region may encompass 0-4 residues
  VARIANT 3: note = Ser or glucosyl serine
  VARIANT 4: note = Ser or glucosyl serine
  VARIANT 5: note = Ser or glucosyl serine
  VARIANT 6..15: note = This region may encompass 1-10 residues
XXXXXGGGGGGGGGG

SEQ ID NO: 569 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid linker
  SITE 10: note = (S)-N-(4-(4,5-diamino-5-oxopentyl)benzyl)-1-hydroxy -3,6,9,12,15,18,21,24-octaoxaheptacosan-27-amide
XXGTXTSDXX

SEQ ID NO: 570 - protein, 7 aa, synthetic construct
  SITE 2: note = (S)-2-amino-3-(2-fluorophenyl)-2-methylpropanoic acid
  SITE 6: note = (S)-2-amino-3-(4'-(4-azidobutoxy)-2'-ethyl-[1,1'-bi phenyl]-4-yl)propanoic acid
  SITE 7: note = (S)-2-amino-5-(3,5-dimethylphenyl)pentanamide
TXTSDXX

SEQ ID NO: 571 - protein, 8 aa, synthetic construct
  SITE 3: note = (S)-2-amino-3-(2-fluorophenyl)-2-methylpropanoic acid
  SITE 7: note = (S)-2-amino-3-(4'-(4-azidobutoxy)-2'-ethyl-[1,1'-bi phenyl]-4-yl)propanoic acid
  SITE 8: note = (S)-2-amino-5-(3,5-dimethylphenyl)pentanamide
GTXTSDXX

SEQ ID NO: 572 - protein, 9 aa, synthetic construct
  SITE 1: note = (S)-2-amino-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-azidobutoxy)-2'-ethyl-[1,1'-bi phenyl]-4-yl)propanoic acid
  SITE 9: note = (S)-2-amino-5-(3,5-dimethylphenyl)pentanamide
XGTXTSDXX

SEQ ID NO: 573 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 574 - protein, 7 aa, synthetic construct
  SITE 2: note = (S)-2-amino-3-(2-fluorophenyl)-2-methylpropanoic acid
  SITE 6: note = (S)-2-amino-3-(4'-(4-aminobutoxy)-2'-ethyl-[1,1'-bi phenyl]-4-yl)propanoic acid
  SITE 7: note = (S)-2-amino-5-(3,5-dimethylphenyl)pentanamide
TXTSDXX

SEQ ID NO: 575 - protein, 8 aa, synthetic construct
  SITE 3: note = (S)-2-amino-3-(2-fluorophenyl)-2-methylpropanoic acid
  SITE 7: note = (S)-2-amino-3-(4'-(4-aminobutoxy)-2'-ethyl-[1,1'-bi phenyl]-4-yl)propanoic acid
  SITE 8: note = (S)-2-amino-5-(3,5-dimethylphenyl)pentanamide
GTXTSDXX

SEQ ID NO: 576 - protein, 9 aa, synthetic construct
  SITE 1: note = (S)-2-amino-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanamide
XGTXTSDXX

SEQ ID NO: 577 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 578 - protein, 10 aa, synthetic construct
  SITE 1: note = 2,2-dimethylmalonic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 579 - protein, 10 aa, synthetic construct
  SITE 1: note = 3-amino-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 580 - protein, 10 aa, synthetic construct
  SITE 1: note = 3-(hydroxyamino)-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 581 - protein, 10 aa, synthetic construct
  SITE 1: note = 2-methyl-2-(1H-pyrazol-5-yl)propanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 582 - protein, 10 aa, synthetic construct
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
SXGTXTSDXX

SEQ ID NO: 583 - protein, 10 aa, synthetic construct
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
AXGTXTSDXX

SEQ ID NO: 584 - protein, 11 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
HAXGTXTSDXX

SEQ ID NO: 585 - protein, 10 aa, synthetic construct
  SITE 1: note = 6-(1H-imidazol-5-yl)hexanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 586 - protein, 10 aa, synthetic construct
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
GXGTXTSDXX

SEQ ID NO: 587 - protein, 11 aa, synthetic construct
  SITE 1: note = 3-(1H-imidazol-4-yl)propanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
XGXGTXTSDXX

SEQ ID NO: 588 - protein, 10 aa, synthetic construct
  SITE 1: note = (R)-1-(2-(1H-imidazol-5-yl)ethyl)-3-methyl-2-oxopyr rolidine-3-carboxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 589 - protein, 10 aa, synthetic construct
  SITE 1: note = (S)-1-(2-(1H-imidazol-5-yl)ethyl)-3-methyl-2-oxopyr rolidine-3-carboxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 590 - protein, 10 aa, synthetic construct
  SITE 1: note = (S)-1-(2-(1H-imidazol-5-yl)ethyl)pyrrolidine-3-carb oxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 591 - protein, 11 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
YAXGTXTSDXX

SEQ ID NO: 592 - protein, 10 aa, synthetic construct
  SITE 1: note = 2-amino-2-methylpropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 593 - protein, 10 aa, synthetic construct
  SITE 1: note = 2-amino-2-methylpropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanamide
XXGTXTSDXX

SEQ ID NO: 594 - protein, 11 aa, synthetic construct
  SITE 2: note = 3-amino-2,2-dimethyl-3-oxopropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanamide
YXXGTXTSDXX

SEQ ID NO: 595 - protein, 9 aa, synthetic construct
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanamide
QGTXTSDXX

SEQ ID NO: 596 - protein, 10 aa, synthetic construct
  SITE 1: note = 3-((2-(1H-imidazol-5-yl)ethyl)amino)-2,2-dimethyl-3 -oxopropanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanamide
XQGTXTSDXX

SEQ ID NO: 597 - protein, 7 aa, synthetic construct
  SITE 2: note = (S)-2-amino-3-(2-fluorophenyl)-2-methylpropanoic acid
  SITE 6: note = (S)-2-amino-3-(2'-ethyl-4'-methoxy-[1,1'-biphenyl]- 4-yl)propanoic acid
  SITE 7: note = (S)-2-amino-5-(4-(4-aminobutoxy)phenyl)pentanamide
TXTSDXX

SEQ ID NO: 598 - protein, 8 aa, synthetic construct
  SITE 3: note = (S)-2-amino-3-(2-fluorophenyl)-2-methylpropanoic acid
  SITE 7: note = (S)-2-amino-3-(2'-ethyl-4'-methoxy-[1,1'-biphenyl]- 4-yl)propanoic acid
  SITE 8: note = (S)-2-amino-5-(4-(4-aminobutoxy)phenyl)pentanamide
GTXTSDXX

SEQ ID NO: 599 - protein, 9 aa, synthetic construct
  SITE 1: note = (S)-2-amino-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid
  SITE 9: note = (S)-2-amino-5-(4-(4-aminobutoxy)phenyl)pentanamide
XGTXTSDXX

SEQ ID NO: 600 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(aminomethyl)phenyl)pentanamide
XXGTXTSDXX

SEQ ID NO: 601 - protein, 5 aa, synthetic construct
  SITE 1: note = N-(tert-butoxycarbonyl)-O-(tert-butyl)-L-serine
XGGGG

SEQ ID NO: 602 - protein, 5 aa, synthetic construct
  SITE 1: note = N-(tert-butoxycarbonyl)-O-(tert-butyl)-L-serine
  SITE 5: note = Glycine attached to (9H-fluoren-9-yl)methanethiol
XGGGG

SEQ ID NO: 603 - protein, 5 aa, synthetic construct
  SITE 1: note = N-(((9H-fluoren-9-yl)methoxy)carbonyl)-O-((2R,3R,4S ,5R,6R)-3,4,5-triacetoxy-6-(acetoxymethyl)tetrahydro-2H-py ran-2-yl)-L-serine
  SITE 5: note = Glycine attached to Phenylmethanol
XGGGG

SEQ ID NO: 604 - protein, 5 aa, synthetic construct
  SITE 1: note = N-(((9H-fluoren-9-yl)methoxy)carbonyl)-O-((2R,3R,4S ,5R,6R)-3,4,5-triacetoxy-6-(acetoxymethyl)tetrahydro-2H-py ran-2-yl)-L-serine
XGGGG

SEQ ID NO: 605 - protein, 6 aa, synthetic construct
LPETGG

SEQ ID NO: 606 - protein, 6 aa, synthetic construct
  SITE 1: note = Pegylated Leucine
LPETGG

SEQ ID NO: 607 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanamide
XXGTXTSDXX

SEQ ID NO: 608 - protein, 9 aa, synthetic construct
  VARIANT 1: note = (S)-2-amino-3-(2H-tetrazol-5-yl)propanoic acid or Glu
  SITE 4: note = (S)-2-amino-3-(2-fluorophenyl)-2-methylpropanoic acid
  SITE 8: note = (S)-2-amino-3-(2'-ethyl-4'-methoxy-[1,1'-biphenyl]- 4-yl)propanoic acid
  SITE 9: note = substituted (S)-2-amino-5-phenylpentanoic acid
XGTXTSDXX

SEQ ID NO: 609 - protein, 5 aa, synthetic construct
  SITE 1: note = pegylated Glycine
GGGGS

SEQ ID NO: 610 - protein, 4 aa, synthetic construct
GGGG

SEQ ID NO: 611 - protein, 8 aa, synthetic construct
GGGGGGGG